(19) United States
(12) Patent Application Publication
(10) Pub. No.: US 2024/0277795 A1
(43) Pub. Date: Aug. 22, 2024

(54) INHIBITORS OF CYSTEINE PROTEASES AND METHODS OF USE THEREOF

(71) Applicant: Pardes Biosciences, Inc., Larkspur, CA (US)

(72) Inventors: Daniel J. Buzard, San Diego, CA (US); Walter Keung, Encinitas, CA (US); Dange Vijay Kumar, San Diego, CA (US); Erik Verner, Belmont, CA (US); Lee D. Arnold, Rancho Santa Fe, CA (US)

(21) Appl. No.: 18/444,009

(22) Filed: Feb. 16, 2024

Related U.S. Application Data
(63) Continuation of application No. PCT/US2022/075185, filed on Aug. 18, 2022.
(60) Provisional application No. 63/392,846, filed on Jul. 27, 2022.
(60) Provisional application No. 63/334,333, filed on Apr. 25, 2022.
(60) Provisional application No. 63/245,996, filed on Sep. 20, 2021.
(60) Provisional application No. 63/234,413, filed on Aug. 18, 2021.

(51) Int. Cl. A61K 38/05; A61K 45/06; A61P 31/14; C07K 1/107

(52) U.S. Cl. CPC A61K 38/05; A61K 45/06; A61P 31/14; C07K 1/1077

(57) ABSTRACT

The disclosure provides compounds with nitrile warheads and their use in treating medical diseases or disorders, such as viral infections. Pharmaceutical compositions and methods of making various compounds with nitrile warheads are provided. The compounds are contemplated to inhibit proteases, such as the 3C, CL- or 3CL-like protease.

### CROSS-REFERENCE TO RELATED APPLICATIONS

This application is a continuation of International Application Number PCT/US2022/075185 filed on Aug. 18, 2022, which claims priority to U.S. Ser. No. 63/234,413, filed Aug. 18, 2021; U.S. Ser. No. 63/245,996, filed Sep. 20, 2021; U.S. Ser. No. 63/334,333, filed Apr. 25, 2022; and U.S. Ser. No. 63/392,846, filed Jul. 27, 2022; the contents of each of which are incorporated herein by reference.

### BACKGROUND

The Coronaviridae family of viruses are enveloped, single-stranded, positive-sense RNA viruses and include 141 species classified into four genera according to their phylogenetic relationships: α-, β-, γ-, and δ-coronavirus. Coronaviruses (CoVs) are zoonotic viruses that infect a variety of animals from whales to birds, bats, cats, and humans. Typically, CoV infection results in mild to moderate respiratory tract infections; however, some CoV species are extremely virulent and can result in widespread fatality. Severe acute respiratory syndrome coronavirus (SARS-CoV) is a human CoV responsible for the first pandemic of the 21^{st} century, infecting over 8,000 people with a 10% mortality rate. Middle East respiratory syndrome coronavirus (MERS-CoV) was identified in November 2012 and had since infected over 1,600 people in 26 countries with a 36% mortality rate. More recently, COVID-19 (SARS CoV2) coronaviruses have raised a global pandemic since first identified in late 2019. Therefore, it is important to identify coronavirus drug targets that can be utilized for the development of broad-spectrum anti-coronaviral therapeutics to combat infections of existing and emerging coronaviruses.

All CoVs express a >800 kDa replicase polyprotein that contains either two or three cysteine proteases, the papain-like protease(s) (PLPpro, nsp3, or PLP1 and PLP2) and the 3C-like protease (3CLpro, nsp5, or Mpro). These proteases process the CoV replicase polyprotein by cleaving it into 16 non-structural proteins, which are responsible for a variety of aspects of CoV replication. The CoV 3CLpro is responsible for processing 11 cleavage sites of within the replicase polyprotein and is essential for CoV replication, making it a highly valuable target for therapeutic development. The overall active site architecture and substrate recognition pockets are structurally conserved across CoV 3CLpros, increasing its attractiveness as a target for the development of broad-spectrum anti-CoV therapeutics. Moreover, high sequence conservation in the vicinity of active site among CoV 3CLpros from different coronavirus subclasses make them an excellent target for the development of broad-spectrum therapeutics for coronavirus infections. Accordingly, the development of CoV 3CLpro inhibitors is a promising path for the treatment of respiratory tract infections and related diseases.

Numerous studies on targeting the immediate zoonotic reservoirs of coronaviruses with small molecule inhibitors have helped inform structure-based design strategies aimed at creating molecular scaffolds that may aid in the development of therapeutic against coronaviral infection; however, small molecule antiviral agents or effective commercially available broad-spectrum therapeutics have not yet been identified. There is a critical need for the development of broad-spectrum CoV therapeutics to overcome the challenges of traditional anti-CoV therapeutic development, as broad-spectrum therapeutics can be rapidly implemented upon zoonotic disease outbreak.

### SUMMARY

The disclosure is directed in part, to viral protease inhibitors. Also disclosed herein are pharmaceutical compositions comprising at least one disclosed compound and a pharmaceutically acceptable carrier.

In an embodiment, disclosed herein is an antiviral compound, comprising a warhead (e.g., a nitrile warhead) covalently bound to a 3C or 3CL protease inhibitor, wherein the antiviral compound covalently binds to a Cys residue of the protease, and wherein the antiviral compound is active against one or more viruses.

For example, disclosed herein is a protease inhibitory compound represented by Formula I:

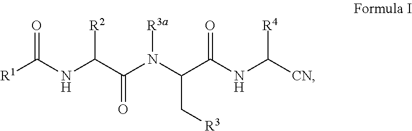

or a pharmaceutically acceptable salt or stereoisomer thereof, wherein R¹ is selected from the group consisting of —CF₃, C₁-C₆alkyl, —C₃-C₆cycloalkyl, —C₁-C₆alkoxy, —O—C₆-C₁₀cycloalkyl, —O-C₃cycloalkyl, —O-C₄cycloalkyl, —O-C₅cycloalkyl, —O—C(R¹⁰)₂-C₆-C₁₀cycloalkyl, —O—C(R¹⁰)₂-phenyl, —O-phenyl, phenyl, —O-(4-1 membered heterocyclyl), —O(CH₂CH₂)ₛ—OR¹¹, —C(R¹²)₂—O—(CH₂CH₂)ₛ—OR¹¹, and 5-6 membered heteroaryl; wherein R¹ may optionally be substituted by one or more substituents each selected from R⁵; R¹⁰ is hydrogen or deuterium;

R¹¹ and R¹² are each independently hydrogen or C₁-C₃alkyl optionally substituted by one or more halogens; R² is selected from the group consisting of C₁-C₆alkyl, CH₂—R²², and C₃-C₆cycloalkyl; wherein R² or R²² may optionally be substituted by one, two, or three substituents each selected from R^{s}; wherein when R¹ is —CF₃. R² is not t-butyl; R¹ is selected from the group consisting of phenyl, naphthyl, and 8-10 membered bicyclic heteroaryl; R^{3a} is hydrogen or C₁-C₃alkyl; R³ is selected from the group consisting of C₁-C₆alkyl, C₃-C₆cycloalkyl, phenyl and 4-6 heterocyclyl and 5-6 membered heteroaryl; wherein R³ may optionally be substituted by one, two, or three substituents each selected from R^{s}; or R³ is —Si(CH₃)₃; or R^{3a} and R³, together with the atoms to which they are attached, may be joined together to form a 4-6 membered monocyclic, 6-10 membered fused bicyclic, or 6-10 membered spirocyclic heterocycle which may optionally be substituted by one, two or three substituents each selected from R⁵; R⁴ is

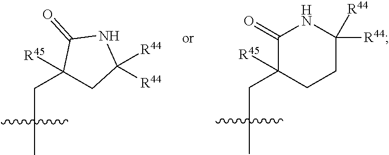

R⁴⁴ is each independently selected from the group consisting of hydrogen, —CH₃ and —CF₃, or two R⁴⁴ groups, together with the carbon to which they are attached, may be joined together to form a cyclopropyl, cyclobutyl, or oxetanyl; R⁴⁵ is hydrogen; or one R⁴⁴ and R⁴⁵, together with the atoms to which they are attached, may be joined together to form a 4-6 membered carbocycle; R^{s} is independently selected for each occurrence from the group consisting of halogen, hydroxyl, cyano, —N(R⁵⁵)₂, —N(R⁵⁵)C(O)R⁵⁵, —C(O)N(R⁵⁵)₂, —OPO(OH)₂, —CO₂H, —SO₂CH₃ —CF₃, —CHF₂, C₁-C₆alkyl, C₁-C₆alkoxy, and phenyl; wherein C₁-C₆alkyl, C₁-C₆alkoxy and phenyl may optionally be substituted by one or more substituents each independently selected from the group consisting of halogen and hydroxyl; and wherein two geminal R^{s} groups, together with the carbon to which they are attached, may be joined together to form a cyclopropyl or cyclobutyl; and R⁵⁵ is hydrogen or C₁-C₃alkyl optionally substituted by one, two or three halogens; or two R⁵⁵ groups, together with the atoms to which they are attached, may be joined together to form a 4-6 membered heterocyclyl, wherein the heterocyclyl may optionally be substituted by one, two or three substituents each independently selected from halogen, hydroxyl and C₁-C₃alkyl (optionally substituted by one or more halogens).

In another example, disclosed herein is Formula II or Formula III:

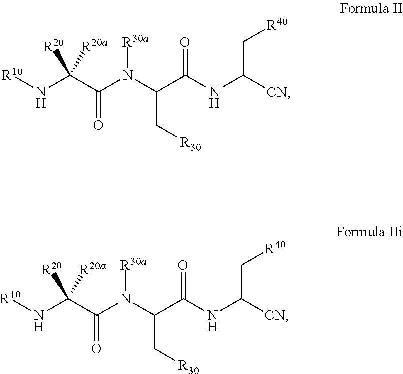

or a pharmaceutically acceptable salt or stereoisomer thereof, wherein: R¹⁰ is selected from the group consisting of hydrogen, —CH₂CF₃, —CH₂CF₂H, —CH(CF₃)₂, —CH(CF₃)CH₃, —C(CH₃)(CF₃)₂, —C(O)NR^{a}R¹⁰⁰, C₃-C₆cycloalkyl, phenyl, and 5-6 membered heteroaryl, wherein phenyl and 5-6 membered heteroaryl may optionally be substituted by or more substituents each independently selected from R⁵⁰; R^{a} is hydrogen or C₁-C₃alkyl; R¹⁰⁰ is selected from the group consisting of hydrogen, C₁-C₆alkyl, C₃-C₁₀cycloalkyl, —C(R¹⁰⁰)₂-C₆-C₁₀cycloalkyl, phenyl, —C(R¹⁰⁰)₂-phenyl, 4-6 membered heterocyclyl, and 5-6 membered heteroaryl; wherein R¹⁰⁰ may optionally be substituted by one or more substituents each selected from R⁵⁰; or R^{a} and R¹⁰⁰, together with the nitrogen to which they are attached, may be joined together to form a 4-6 membered monocyclic or a 6-10 membered spirocyclic heterocyclyl optionally substituted by one or more halogens; R¹⁰⁰ is hydrogen or deuterium; R²⁰ is selected from the group consisting of C₁-C₆alkyl CH₂-R^{HO}; and C₃-C₆cycloalkyl, wherein R²⁰ or R² may optionally be substituted by one, two, or three substituents each selected from R⁵⁰; R is H; or R²⁰ and R^{20a}, together with the carbon to which they are attached, may be joined together to form a C₃-C₆cycloalkyl or 4-6 membered monocyclic heterocyclyl; R²²⁰ is selected from the group consisting of phenyl, naphthyl, and 8-10 membered bicyclic heteroaryl; R is hydrogen or C₁-C₃alkyl; R³⁰ is selected from the group consisting of C₁-C₆alkyl, C₃-C₆cycloalkyl, phenyl, 4-6 heterocyclyl and 5-6 membered heteroaryl; wherein R³⁰ may optionally be substituted by one, two, or three substituents each selected from R⁵⁰; or R³⁰ is —Si(CH₃)₃; or R³ and R³⁰, together with the atoms to which they are attached, may be joined together to form a 4-6 membered monocyclic, 6-10 membered fused bicyclic, or 6-10 membered spirocyclic heterocyclyl which may optionally be substituted by one, two or three substituents each selected from R⁵⁰; R⁴⁰ is

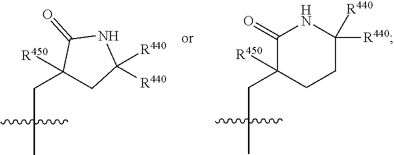

R⁴⁴⁰ is each independently selected from the group consisting of hydrogen, —CH₃ and —CF₃, or two R⁴⁴⁰ groups, together with the carbon to which they are attached, may be joined together to form a cyclopropyl, cyclobutyl, or oxetanyl; R⁴⁵⁰ is hydrogen; or one R⁴⁴⁰ and R⁴⁵⁰, together with the atoms to which they are attached, may be joined together to form a 4-6 membered carbocycle; R⁵⁰ is independently selected for each occurrence from the group consisting of halogen, hydroxyl, cyano, —N(R⁵⁵⁰)₂, —N(R⁵⁵⁰)C(O)R⁵⁵⁰, —C(O)N(R⁵⁵⁰)₂, —OPO(OH)₂, —CO₂H, —SO₂CH₃ —CF₃, —CHF₂, C₁-C₆alkyl, C₁-C₆alkoxy, and phenyl; wherein C₁-C₆alkyl, C₁-C₆alkoxy and phenyl may optionally be substituted by one or more substituents each independently selected from the group consisting of halogen and hydroxyl; and R^{5S°} is hydrogen or C₁-C₃alkyl optionally substituted by one, two or three halogens; or two R⁵⁵ groups, together with the atoms to which they are attached, may be joined together to form a 4-6 membered heterocyclyl, wherein the heterocycicyl may optionally be substituted by one, two or three substituents each independently selected from halogen, hydroxyl and C₁-C₃alkyl(optionally substituted by one or more halogens).

In another example, disclosed herein is Formula III:

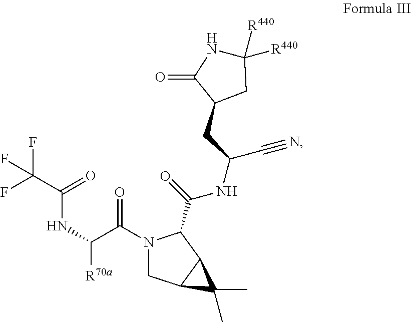

or a pharmaceutically acceptable salt or stereoisomer thereof, wherein R⁴⁰⁰ is each independently selected from the group consisting of hydrogen, —CH₃ and —CF₃, or two R⁴⁴⁰ groups, together with the carbon to which they are attached, may be joined together to form a cyclopropyl, cyclobutyl, or oxetanyl; R^{70a} is selected from the group consisting of C₁₋₃alkylene-C₃₋₅cycloalkyl, C₁₋₆alkyl, C₃₋₅cycloalkyl, C₁₋₃alkyl-O— R⁵⁰¹ and a 4-6 membered heterocyclyl having one ring oxygen; wherein R^{70a} may optionally be substituted by one, two, three substituents each independently selected from R⁵⁰⁰; R⁵⁰¹ is selected from the group consisting of C₁₋₆alkyl, C₁₋₆alkenyl, phenyl, C₃-C₆cycloalkyl, 4-6 membered heterocyclyl having one ring oxygen and 5-6 membered heteroaryl, wherein R⁵⁰¹ may be optionally substituted by one two, three or more substituents each independently selected from the group consisting of halo, cyano, methyl, ethyl, C₁₋₆alkoxy, CF₃, —CHF₂, —CH₂—CF₃ and C₃₋₆cycloalkyl; and R⁵⁰⁰ is independently selected for each occurrence from the group consisting of halogen, cyano, —CF₃, —CHF₂, C₁-C₂alkyl, and C₂₋₆alkenyl.

In another example, disclosed herein is a protease inhibitory compound represented by Formula X:

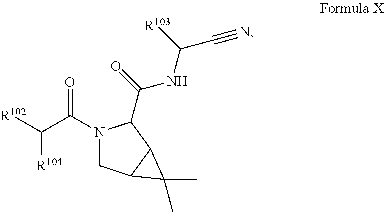

or a pharmaceutically acceptable salt, and/or a stereoisomer thereof, wherein: R¹⁰⁴ is selected from the group consisting of

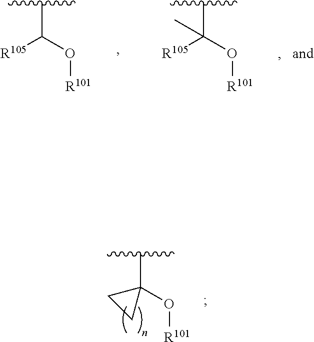

wherein R¹⁰⁵ is C₁-C₆alkyl, and n is 1, 2, or 3; R¹⁰¹ is selected from the group consisting of C₁-C₆alkyl, C₂-C₆alkenyl, C₃-C₆cycloalkyl, and C₃-C₆cycloalkenyl, wherein R¹⁰¹ may optionally be substituted by one, two, three, or four substituents each independently selected from R^{D}; R^{D} is selected from the group consisting of halogen, C₁-C₆alkyl, C₁-C₆alkoxy, C₃-C₆cycloalkyl, and cyano, wherein the alkyl may optionally be substituted by one or more halo; R¹⁰² is —NR^{m}(C═O)R^{m}; R^{m} is, for each occurrence, selected from the group consisting of H, C₁₋₆alkyl and C₃-C₆cycloalkyl, wherein the alkyl or cycloalkyl is optionally substituted by one, two or three halo or —CF₃; R¹⁰³ is

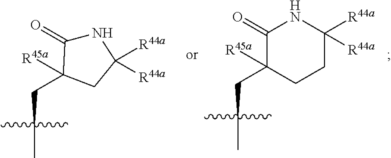

R^{44a} is each independently selected from the group consisting of hydrogen, —CH₃ and —CF₃, or two R^{44a} groups, together with the carbon to which they are attached, may be joined together to form a cyclopropyl, cyclobutyl, or oxetanyl; and R^{45a} is H or C₁-C₆alkyl.

In another aspect of this disclosure, provided herein is a protease inhibitory compound represented by Formula XI:

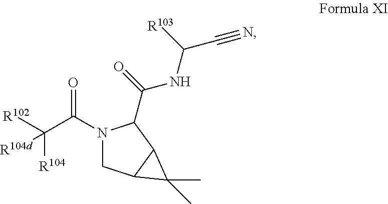

or a pharmaceutically acceptable salt, and/or a stereoisomer thereof, wherein: R¹⁰⁴ is selected from the group consisting of

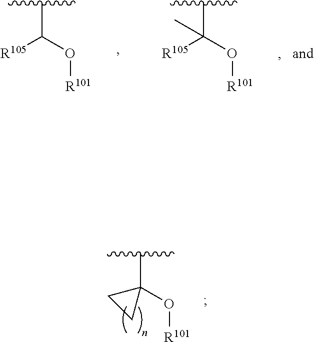

wherein R¹⁰⁵ is C₁-C₂alkyl, and n is 1, or 2; R¹⁰⁴ is H; or R^{104a} and R^{104d}, together with the carbon atom to which they are attached, may be joined to form a 4-6 membered heterocyclyl; R¹⁰¹ is selected from the group consisting of C₁-C₆alkyl, C₂-C₆alkenyl, C₃-C₆cycloalkyl, and C₃-C₆cycloalkenyl, wherein R¹⁰¹ may optionally be substituted by one, two, three, or four substituents each independently selected from R^{F}; or R¹⁰¹ and R¹⁰⁵ may be joined, together with the atoms to which they are attached, to form a 4-6 membered heterocyclyl, wherein the heterocyclyl may optionally be substituted by one, two, three, or four substituents each independently selected from the group consisting of halogen, and C₁-C₆alkyl; R^{F} is selected from the group consisting of halogen, C₁-C₂alkyl, C₁-C₆alkoxy, C₃-C₆cycloalkyl, and cyano, wherein the alkyl may optionally be substituted by one or more halo; R¹⁰² is selected from —NR^{m}(C═O)Rⁿ and

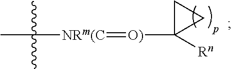

p is selected from 1 and 2; R^{m} is H; Rⁿ is selected from C₁₋₂alkyl and C₃-C₄cycloalkyl, wherein the alkyl one, two or three halo or —CF₃; R¹⁰³ is selected from

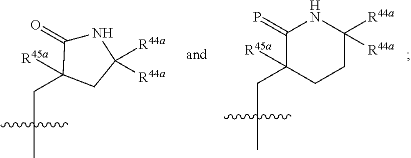

R^{44a} is each independently selected from the group consisting of hydrogen, —CH₃ and —CF₃, or two R^{44a} groups, together with the carbon to which they are attached, may be joined together to form a cyclopropyl, cyclobutyl, or oxetanyl; and R^{45a} is H, CH₃ or halo.

In another aspect of the disclosure, provided herein is a protease inhibitory compound represented by Formula XII:

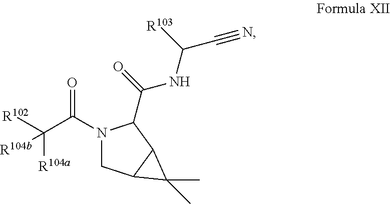

or a pharmaceutically acceptable salt, and/or a stereoisomer thereof, wherein: R^{104a} is selected from the group consisting of

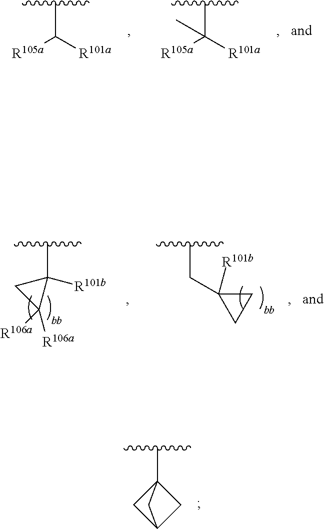

wherein R^{105a} is C₁-C₂alkyl; bb is selected from 1 and 2; R^{106a} is each independently selected from H and halo; R^{101a} and R^{101b} are each independently selected from the group consisting of H, halo, C₁-C₆alkyl, C₂-C₆alkenyl, C₃-C₆cycloalkyl, and C₃-C₆cycloalkenyl, wherein the alkyl, alkenyl, cycloalkyl, or cycloalkenyl may optionally be substituted by one, two, three, or four substituents each independently selected from the group consisting of halogen, C₁-C₆alkyl, C₁-C₆alkoxy, C₃-C₆cycloalkyl, phenyl, and pyridinyl, and cyano, wherein the alkyl may optionally be substituted by one or more halo; R^{104b} represents H;
alternatively, R^{104a} and R^{104b}, together with the carbon atom to which they are attached, may be joined to form a C₃-C₆cycloalkyl, wherein the cycloalkyl ring may be optionally substituted by one, two, three, or four substituents each independently selected from R^{P}; wherein R^{P} is selected from the group consisting of halogen, C₁-C₆alkyl, C₁-C₆alkenyl, C₁-C₆alkoxy, C₃-C₆cycloalkyl, and cyano, wherein the alkyl may optionally be substituted by one or more halo; R¹⁰² is selected from the group consisting of —NR^{m}(C═O)Rⁿ,

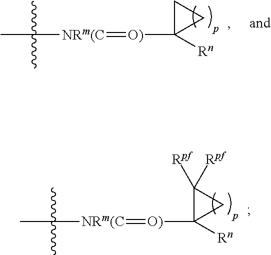

p is selected from 1 and 2; R^{m} is H; R is selected from C₁₋₂alkyl and C₃-C₄cycloalkyl, wherein the alkyl or cycloalkyl is optionally substituted by one, two or three methyl, halo or —CF₃; R^{Pf} is each independently selected from H and halo; R¹⁰³ is selected from and

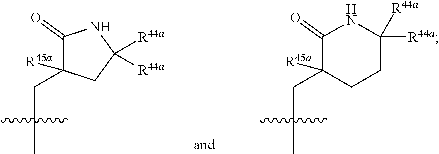

R^{44a} is each independently selected from the group consisting of hydrogen, —CH₃ and —CF₃, or two R^{44a} groups, together with the carbon to which they are attached, may be joined together to form a cyclopropyl, cyclobutyl, or oxetanyl; and R^{45a} is each independently selected from the group consisting of H, CH₃ and halo.

Also provided herein are methods of ameliorating or treating a viral infection in a patient in need thereof, comprising administering to the patient a therapeutically effective amount of any of the compounds described herein.

In some embodiments, provided herein are methods of inhibiting transmission of a virus, a method of inhibiting viral replication, a method of minimizing expression of viral proteins, or a method of inhibiting virus release, comprising administering a therapeutically effective amount of a compound described herein to a patient suffering from the virus, and/or contacting an effective amount of a compound described herein with a virally infected cell.

### DETAILED DESCRIPTION

The features and other details of the disclosure will now be more particularly described. Before further description of the present disclosure, certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

The term “treating” includes any effect, e.g., lessening, reducing, modulating, or eliminating, that results in the improvement of the condition, disease, disorder and the like, including a reduction of viral shedding in asymptomatic individuals and prophylaxis of exposed individuals, independent of symptoms.

The term “alkyl” as used herein refers to a saturated straight or branched hydrocarbon. Exemplary alkyl groups include, but are not limited to, straight or branched hydrocarbons of 1-6, 1-4, or 1-3 carbon atoms, referred to herein as C₁₋₆alkyl, C₁₋₄alkyl, and C₁₋₃alkyl, respectively. Exemplary alkyl groups include, but are not limited to, methyl, ethyl, propyl, isopropyl, 2-methyl-1-butyl, 3-methyl-2-butyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, etc.

The term “alkenyl” as used herein refers to an unsaturated straight or branched hydrocarbon having at least one carbon-carbon double bond. Exemplary alkenyl groups include, but are not limited to, a straight or branched group of 2-6 or 3-4 carbon atoms, referred to herein as C₁-C₅alkenyl, C₂-C₆alkenyl, and C₃-C₄alkenyl, respectively. Exemplary alkenyl groups include, but are not limited to, vinyl, allyl, butenyl, pentenyl, etc.

The term “alkynyl” as used herein refers to an unsaturated straight or branched hydrocarbon having at least one carbon-carbon triple bond. Exemplary alkynyl groups include, but are not limited to, straight or branched groups of 2-6, or 3-6 carbon atoms, referred to herein as C₂₋₆alkynyl, and C₃₋₆alkynyl, respectively. Exemplary alkynyl groups include, but are not limited to, ethynyl, propynyl, butynyl, pentynyl, hexynyl, methylpropynyl, etc.

The term “alkoxy” as used herein refers to a straight or branched alkyl group attached to oxygen (alkyl-O—). The term “alkoxy” as used herein also refers to a cyclic group attached to oxygen (alkyl-O—). Exemplary alkoxy groups include, but are not limited to, alkoxy groups of 1-6 or 2-6 carbon atoms, referred to herein as C₁-C₅alkoxy, C₁-C₆alkoxy, and C₂-C₆alkoxy, respectively. Exemplary alkoxy groups include, but are not limited to methoxy, ethoxy, isopropoxy, cyclopropoxy, cyclobutoxy, etc.

The term “aryl” refers to a radical of a monocyclic or polycyclic (e.g., bicyclic or tricyclic) 4n+2 aromatic ring system (e.g., having 6, 10, or 14 t electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl. Examples of representative substituted aryls include the following

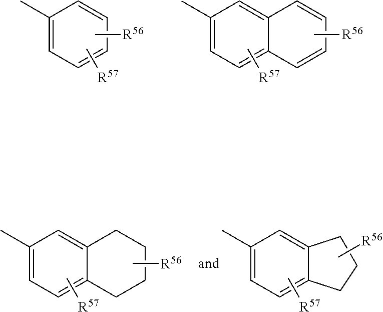

wherein one of R⁵⁶ and R⁵⁷ may be hydrogen and at least one of R⁵⁶ and R⁵⁷ is each independently selected from C₁-C₈ alkyl, C₁-C₈ haloalkyl, 4-10 membered heterocyclyl, alkanoyl, C₁-C₈ alkoxy, heteroaryloxy, alkylamino, arylamino, heteroarylamino, NR⁵⁸COR⁵⁹, NR⁵⁸SOR⁵⁹ NR⁵¹SO₂R⁵⁹, COOalkyl, COOaryl, CONR⁵⁸R⁵⁹, CONR⁵⁸OR⁵⁹, NR⁵⁸R⁵⁹, SO₂NR⁵⁸R⁵⁹, S-alkyl, SOalkyl, SO₂alkyl, Saryl, SOaryl, SO₂aryl; or R⁵⁶ and R⁵⁷ may be joined to form a cyclic ring (saturated or unsaturated) from 5 to 8 atoms, optionally containing one or more heteroatoms selected from the group N, O, or S. R⁶⁰ and R⁶¹ are each independently hydrogen, C₁-C₈ alkyl, C₁-C₄ haloalkyl, C₃-C₁₀ cycloalkyl, 4-10 membered heterocyclyl, C₆-C₁₀ aryl, substituted C₆-C₁₀ aryl, 5-10 membered heteroaryl, or substituted 5-10 membered heteroaryl.

The term “carbonyl” as used herein refers to the radical —C(O)—.

The term “cyano” as used herein refers to the radical —CN.

The terms “cycloalkyl” or a “carbocyclic group” as used herein refers to a saturated or partially unsaturated hydrocarbon group of, for example, 3-6, or 4-6 carbons, referred to herein as C₃-C₁₀cycloalkyl, C₃₋₆cycloalkyl or C₄₋₆cycloalkyl, respectively. Exemplary cycloalkyl groups include, but are not limited to, cyclohexyl, cyclopentyl, cyclopentenyl, cyclobutyl or cyclopropyl. The terms “cycloalkyl” or a “carbocyclic group” as used herein include bridged cycloalkyl or spirocyclic cycloalkyl.

The terms “halo” or “halogen” as used herein refer to F, Cl, Br, or I.

The terms “haloalkyl” as used herein refers to an alkyl radical in which the alkyl group is substituted with one or more halogens. Typical haloalkyl groups include, but are not limited to, trifluoromethyl (i.e. CF₃), difluoromethyl, fluoromethyl, chloromethyl, dichloromethyl, dibromoethyl, tribromomethyl, tetrafluoroethyl, and the like. Exemplary haloalkyl groups include, but are not limited to, straight or branched hydrocarbons of 1-6, 1-4, or 1-3 carbon atoms substituted with a halogen (i.e. Cl, F, Br and I), referred to herein as C₁₋₆haloalkyl, C₁₋₄ haloalkyl, and C₁₋₃haloalkyl, respectively.

The term “hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g., heteroalkyl, cycloalkyl, e.g., heterocyclyl, aryl, e.g., heteroaryl, cycloalkenyl, e.g., cycloheteroalkenyl, and the like having from 1 to 5, and particularly from 1 to 3 heteroatoms.

The terms “heteroaryl” or “heteroaromatic group” as used herein refers to an aromatic 5-10 membered ring system containing one or more heteroatoms, for example one to three heteroatoms, such as nitrogen, oxygen, and sulfur. The term may also be used to refer to a 5-7 membered monocyclic heteroaryl or an 8-10 membered bicyclic heteroaryl. Where possible, said heteroaryl ring may be linked to the adjacent radical though carbon or nitrogen. Examples of heteroaryl rings include but are not limited to furan, thiophene, pyrrole, pyrrolopyridine, indole, thiazole, oxazole, isothiazole, isoxazole, imidazole, benzoimidazole, imidazopyridine, pyrazole, triazole, pyridine or pyrimidine, etc.

Examples of representative heteroaryls include the following:

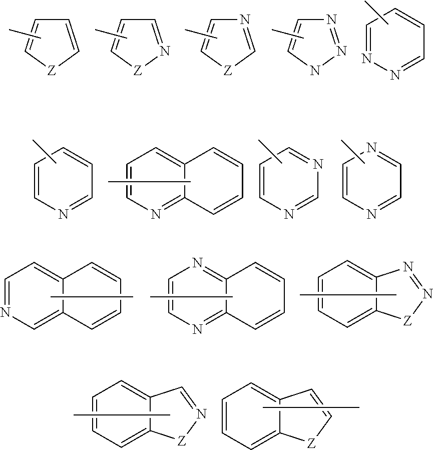

wherein each Z is selected from carbonyl, N, NR⁶⁵, O, and S; and R⁶⁵ is each independently hydrogen, C₁-C₈ alkyl, C₃-C₁₀ cycloalkyl, 4-10 membered heterocyclyl, C₆-C₁₀ aryl, or 5-10 membered heteroaryl.

The terms “heterocyclyl,” “heterocycle,” or “heterocyclic group” are art-recognized and refer to saturated or partially unsaturated 4-10 membered ring structures, whose ring structures include one to three heteroatoms, such as nitrogen, oxygen, and sulfur. Where possible, heterocyclyl rings may be linked to the adjacent radical through carbon or nitrogen. The term may also be used to refer to 4-10 membered saturated or partially unsaturated ring structures that are bridged, fused or spirocyclic ring structures, whose ring structures include one to three heteroatoms, such as nitrogen, oxygen, and sulfur. Examples of heterocyclyl groups include, but are not limited to, pyrrolidine, piperidine, morpholine, thiomorpholine, piperazine, oxetane, azetidine, tetrahydrofuran, dihydrofuran, dihydropyran, tetrahydropyran, etc. In some embodiments, the heterocycle is a spiro heterocycle (e.g., 2,8-diazaspiro[4.5]decane). In some embodiments, the heterocycle is a bridged heterocycle (e.g., octahydro-1 H-4,7-methanoisoindole). “Spiro heterocyclyl,” or “spiro heterocycle” refers to a polycyclic heterocyclyl with rings connected through one common atom (called a spiro atom), wherein the rings have one or more heteroatoms selected from the group consisting of N, O, and S(0)ₘ (wherein m is an integer of 0 to 2) as ring atoms. Representative examples of heterocyclyl include, for example:

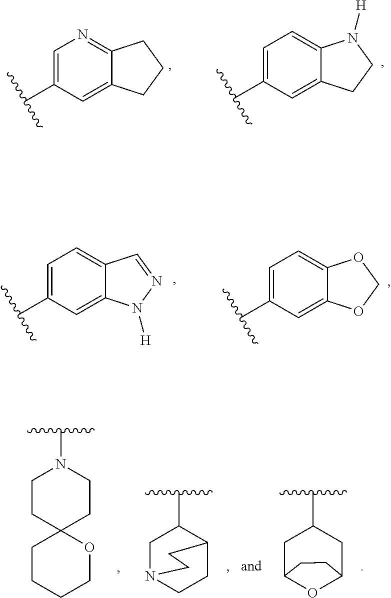

The terms “hydroxy” and “hydroxyl” as used herein refers to the radical —OH.

The term “oxo” as used herein refers to the radical ═O.

“Pharmaceutically or pharmacologically acceptable” include molecular entities and compositions that do not produce an adverse, allergic or other untoward reaction when administered to an animal, or a human, as appropriate. For human administration, preparations should meet sterility, pyrogenicity, and general safety and purity standards as required by FDA Office of Biologics standards.

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable carriers.

“Individual,” “patient,” or “subject” are used interchangeably and include any animal, including mammals, preferably mice, rats, other rodents, rabbits, dogs, cats, swine, cattle, sheep, horses, or primates, and most preferably humans. The compounds of the disclosure can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). “Modulation” includes antagonism (e.g., inhibition), agonism, partial antagonism and/or partial agonism.

In the present specification, the term “therapeutically effective amount” means the amount of the subject compound that will elicit the biological or medical response of a tissue, system or animal, (e.g. mammal or human) that is being sought by the researcher, veterinarian, medical doctor or other clinician. The compounds of the disclosure are administered in therapeutically effective amounts to treat a disease. Alternatively, a therapeutically effective amount of a compound is the quantity required to achieve a desired therapeutic and/or prophylactic effect.

The term “pharmaceutically acceptable salt(s)” as used herein refers to salts of acidic or basic groups that may be present in compounds used in the compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that are acidic in nature are capable of forming base salts with various pharmacologically acceptable cations. Examples of such salts include alkali metal or alkaline earth metal salts, particularly calcium, magnesium, sodium, lithium, zinc, potassium, and iron salts. Compounds included in the present compositions that include a basic or acidic moiety may also form pharmaceutically acceptable salts with various amino acids. The compounds of the disclosure may contain both acidic and basic groups; for example, one amino and one carboxylic acid group. In such a case, the compound can exist as an acid addition salt, a zwitterion, or a base salt.

The compounds of the disclosure may contain one or more chiral centers and, therefore, exist as stereoisomers. The term “stereoisomers” when used herein consist of all enantiomers or diastereomers. These compounds may be designated by the symbols “(+),” “(−),” “R” or “S,” depending on the configuration of substituents around the stereogenic carbon atom, but the skilled artisan will recognize that a structure may denote a chiral center implicitly. The present disclosure encompasses various stereoisomers of these compounds and mixtures thereof. Mixtures of enantiomers or diastereomers may be designated “(±)” in nomenclature, but the skilled artisan will recognize that a structure may denote a chiral center implicitly.

The compounds of the disclosure may contain one or more double bonds and, therefore, exist as geometric isomers resulting from the arrangement of substituents around a carbon-carbon double bond. The symbol denotes a bond that may be a single, double or triple bond as described herein. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers. Substituents around a carbon-carbon double bond alternatively can be referred to as “cis” or “trans,” where “cis” represents substituents on the same side of the double bond and “trans” represents substituents on opposite sides of the double bond.

Compounds of the disclosure may contain a carbocyclic or heterocyclic ring and therefore, exist as geometric isomers resulting from the arrangement of substituents around the ring. The arrangement of substituents around a carbocyclic or heterocyclic ring are designated as being in the “Z” or “E” configuration wherein the terms “Z” and “E” are used in accordance with IUPAC standards. Unless otherwise specified, structures depicting carbocyclic or heterocyclic rings encompass both “Z” and “E” isomers. Substituents around a carbocyclic or heterocyclic rings may also be referred to as “cis” or “trans”, where the term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

Individual enantiomers and diastereomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns or (4) kinetic resolution using stereoselective chemical or enzymatic reagents. Racemic mixtures can also be resolved into their component enantiomers by well known methods, such as chiral-phase liquid chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio- and diastereoselective transformations, and may involve the use of chiral auxiliaries. For examples, see Carreira and Kvaerno, Classics in Stereoselective Synthesis, Wiley-VCH: Weinheim, 2009.

The compounds disclosed herein can exist in solvated as well as unsolvated forms with pharmaceutically acceptable solvents such as water, ethanol, and the like, and it is intended that the disclosure embrace both solvated and unsolvated forms. In one embodiment, the compound is amorphous. In one embodiment, the compound is a single polymorph. In another embodiment, the compound is a mixture of polymorphs. In another embodiment, the compound is in a crystalline form.

The disclosure also embraces isotopically labeled compounds of the disclosure which are identical to those recited herein, except that one or more atoms are replaced by an atom having an atomic mass or mass number different from the atomic mass or mass number usually found in nature. Examples of isotopes that can be incorporated into compounds of the disclosure include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. For example, a compound of the disclosure may have one or more H atom replaced with deuterium.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the disclosure can generally be prepared by following procedures analogous to those disclosed in the examples herein by substituting an isotopically labeled reagent for a non-isotopically labeled reagent.

The term “prodrug” refers to compounds that are transformed in vivo to yield a disclosed compound or a pharmaceutically acceptable salt, hydrate or solvate of the compound. The transformation may occur by various mechanisms (such as by esterase, amidase, phosphatase, oxidative and or reductive metabolism) in various locations (such as in the intestinal lumen or upon transit of the intestine, blood or liver). Prodrugs are well known in the art (for example, see Rautio, Kumpulainen, et al, Nature Reviews Drug Discovery 2008, 7, 255). For example, if a compound of the disclosure or a pharmaceutically acceptable salt, hydrate or solvate of the compound contains a carboxylic acid functional group, a prodrug can comprise an ester formed by the replacement of the hydrogen atom of the acid group with a group such as (C₁₋₈)alkyl, (C₂₋₁₂)alkylcarbonyloxymethyl, 1-(alkylcarbonyloxy)ethyl having from 4 to 9 carbon atoms, 1-methyl-1-(alkylcarbonyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁₋₂)alkylamino(C₂₋₃)alkyl (such as β-dimethylaminoethyl), carbamoyl-(C₁₋₂)alkyl, N,N-di(C₁₋₂)alkylcarbamoyl-(C₁₋₂)alkyl and piperidino-, pyrrolidino- or morpholino(C₂₋₃)alkyl.

Similarly, if a compound of the disclosure contains an alcohol functional group, a prodrug can be formed by the replacement of the hydrogen atom of the alcohol group with a group such as (C₁₋₆)alkylcarbonyloxymethyl, 1-((C₁₋₆)alkylcarbonyloxy)ethyl, 1-methyl-1-((C₁₋₆)alkylcarbonyloxy)ethyl (C₁₋₆)alkoxycarbonyloxymethyl, N—(C₁₋₆)alkoxycarbonylaminomethyl, succinoyl, (C₁₋₆)alkylcarbonyl, α-amino(C₁₋₄)alkylcarbonyl, arylalkylcarbonyl and α-aminoalkylcarbonyl, or α-aminoalkylcarbonyl-α-aminoalkylcarbonyl, where each α-aminoalkylcarbonyl group is independently selected from the naturally occurring L-amino acids, P(O)(OH)₂, —P(O)(O(C₁₋₆)alkyl)₂ or glycosyl (the radical resulting from the removal of a hydroxyl group of the hemiacetal form of a carbohydrate).

If a compound of the disclosure incorporates an amine functional group, a prodrug can be formed, for example, by creation of an amide or carbamate, an N-alkylcarbonyloxyalkyl derivative, an (oxodioxolenyl)methyl derivative, an N-Mannich base, imine or enamine. In addition, a secondary amine can be metabolically cleaved to generate a bioactive primary amine, or a tertiary amine can metabolically cleaved to generate a bioactive primary or secondary amine. For examples, see Simplício, et al., Molecules 2008, 13, 519 and references therein.

The term “warhead” or “warhead group” as used herein refers to a functional group present on a compound wherein that functional group is capable of reversibly or irreversibly participating in a reaction with a protein, e.g., 3C or 3CL protease (e.g., with a cysteine on the protease such as Cys 145). Warheads may, for example, form covalent bonds with the protein, or may create stable transition states, or be a reversible or an irreversible alkylating agent. For example, the warhead moiety can be a functional group on an inhibitor that can participate in a bond-forming reaction, wherein a new covalent bond is formed between a portion of the warhead and a donor, for example an amino acid residue of a protein. In embodiments, the warhead is an electrophile and the “donor” is a nucleophile such as the side chain of a cysteine residue. As provided herein, a warhead may include a nitrile group. For example, nitriles may be reversible covalent warheads for cysteine protease inhibition. For example, where the mechanism of action may involve a formation of reversible covalent bond between the nitrile and the active cysteine to form a thioimidate adduct. Reaction of cysteine of glutathione or other proteins is generally reversible, while the reaction with cysteine or aminoethylthiols generally irreversibly forms a thiazolidine adduct. It can be appreciated that contemplated compounds herein may be a reversible or an irreversible inhibitor. It will be appreciated to one of skilled in the art that the compounds disclosed herein that include the warheads above also contemplate the precursors to those compounds, for example, where a cyano moiety involved in a warheads may be replaced with e.g., a halo moiety.

It will be appreciated to one of skilled in the art that the compounds disclosed herein can also irreversibly bind, or may otherwise inhibit e.g., a virus protein via any other mechanism of action.

The term “inhibitor” as used herein refers to a compound that binds to and/or inhibits a target protease with measurable affinity.

The term “reversible” or “reversible inhibitor” as used herein refers to a protease inhibitor that associates with a protease in such a way as to inhibit the activity of the protease while the protease and inhibitor are bound, but does not associate with a protease in such a way as to inhibit the activity of the protease when the protease and inhibitor are no longer bound. Reversible inhibitors can effect inhibition by competing with substrate for binding to the active site of the protease (competitive reversible inhibitor), or by associating with the protease bound to its substrate in a way to make the complex inactive (uncompetitive reversible inhibitor), or by associating with the protease and/or protease-substrate complex in a way that inhibits the activity of either and/or both.

As used herein, the term “irreversible” or “irreversible inhibitor” refers to an inhibitor (i.e. a compound) that is able to be covalently bonded to a target protease in a substantially non-reversible manner. An irreversible inhibitor will remain substantially bound to the target protease once covalent bond formation has occurred. Irreversible inhibitors usually display time dependency, whereby the degree of inhibition increases with the time with which the inhibitor is in contact with the enzyme. In certain embodiments, an irreversible inhibitor will remain substantially bound to target protease once covalent bond formation has occurred and will remain bound for a time period that is longer than the life of the protein.

### I. Viral Protease Inhibitor Compounds

The disclosure is directed to, in part, compounds that inhibit a viral protease. Examples of viral proteases include, but not limited to, Cathepsin K, coronavirus main protease (Mpro), Caspase 3, Calpain 1, and Cathepsin S. Accordingly, in various embodiments, a compound of the present disclosure (e.g. a compound of Formula I or II) is a viral protease inhibitor, wherein the viral protease is selected from the group consisting of Cathepsin K, coronavirus main protease (Mpro), Caspase 3, Calpain 1, and Cathepsin S. In certain embodiments, the viral protease is a coronavirus main protease (Mpro). In some embodiments, the viral protease is Cathepsin K. In some embodiments, the viral protease is Caspase 3. In some embodiments, the viral protease is Calpain 1. In some embodiments, the viral protease is Cathepsin S.

For example, disclosed herein is a protease inhibitory compound represented by Formula I:

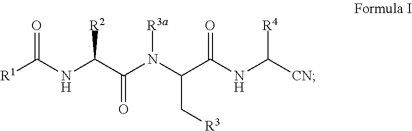

or a pharmaceutically acceptable salt or stereoisomer thereof, wherein R¹ is selected from the group consisting of —CF₃, C₁-C₆alkyl, —C₃-C₆cycloalkyl, —C₁-C₆alkoxy, —O—C₆-C₁₀cycloalkyl, —O-C₃cycloalkyl, —O-C₄cycloalkyl, —O-C₅cycloalkyl, —O—C(R¹⁰)₂-C₆-C₁₀cycloalkyl, —O—C(R¹⁰)₂-phenyl, —O-phenyl, phenyl, —O-(4-1 membered heterocyclyl), —O(CH₂CH₂)ₛ—OR¹¹, —C(R¹²)₂—O—(CH₂CH₂)ₛ—OR¹¹, and 5-6 membered heteroaryl; wherein R¹ may optionally be substituted by one or more substituents each selected from R⁵; s is selected from 1 and 2; R¹⁰ is selected from hydrogen and deuterium; R¹¹ and R¹² are each independently hydrogen or C₁-C₃alkyl optionally substituted by one or more halogens; R² is selected from the group consisting of C₁-C₆alkyl, CH₂-R²², and C₃-C₆cycloalkyl; wherein R² or R²² may optionally be substituted by one, two, or three substituents each selected from R⁵; wherein when R¹ is —CF₃. R² is not t-butyl; R²² is selected from the group consisting of phenyl, naphthyl, and 8-10 membered bicyclic heteroaryl; R¹ is selected from hydrogen and C₁-C₃alkyl; R³ is selected from the group consisting of C₁-C₆alkyl, C₃-C₆cycloalkyl, phenyl and 4-6 heterocyclyl and 5-6 membered heteroaryl; wherein R³ may optionally be substituted by one, two, or three substituents each selected from R^{s}; or R³ is —Si(CH₃)₃; or R^{3a} and R³, together with the atoms to which they are attached, may be joined together to form a 4-6 membered monocyclic, 6-10 membered fused bicyclic, or 6-10 membered spirocyclic heterocycle which be substituted by one, two or three substituents each selected from R⁵; R is

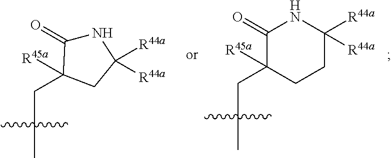

R⁴⁴ is each independently selected from the group consisting of hydrogen, —CH₃ and —CF₃, or two R⁴⁴ groups, together with the carbon to which they are attached, may be joined together to form a cyclopropyl, cyclobutyl, or oxetanyl; R⁴⁵ is selected from hydrogen and C₁-C₃alkyl; or one R⁴⁴ and R⁴⁵, together with the atoms to which they are attached, may be joined together to form a 4-6 membered carbocycle; R⁵ is independently selected for each occurrence from the group consisting of halogen, hydroxyl, cyano, —N(R⁵⁵)₂, —N(R⁵⁵)C(O)R⁵⁵, —C(O)N(R⁵⁵)₂, —OPO(OH)₂, —CO₂H, —SO₂CH₃ —CF₃, —CHF₂, C₁-C₆alkyl, C₁-C₆alkoxy, and phenyl; wherein C₁-C₆alkyl, C₁-C₆alkoxy and phenyl may optionally be substituted by one or more substituents each independently selected from the group consisting of halogen and hydroxyl; and wherein two geminal R⁵ groups, together with the carbon to which they are attached, may be joined together to form a cyclopropyl or cyclobutyl; and R⁵⁵ is selected from hydrogen and C₁-C₃alkyl, wherein the alkyl is optionally substituted by one, two or three halogens; or two R⁵⁵ groups, together with the atoms to which they are attached, may be joined together to form a 4-6 membered heterocyclyl, wherein the heterocycicyl may optionally be substituted by one, two or three substituents each independently selected from the group consisting of halogen, hydroxyl and C₁-C₃alkyl (optionally substituted by one or more halogens).

In some embodiments, R⁴ is

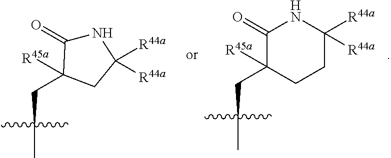

In some embodiments, disclosed herein is a protease inhibitory compound represented by Formula I:

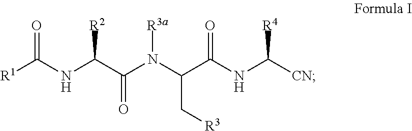

or a pharmaceutically acceptable salt or stereoisomer thereof, wherein R¹ is selected from the group consisting of —CF₃, C₁-C₆alkyl, —C₃-C₆cycloalkyl, —C₁-C₆alkoxy, —O—C₆-C₁₀cycloalkyl, —O-C₃cycloalkyl, —O-C₄cycloalkyl, —O-C₅cycloalkyl, —O—C(R¹⁰)₂-C₆-C₁₀cycloalkyl, —O—C(R¹⁰)₂-phenyl, —O-phenyl, phenyl, —O-(4-1 membered heterocyclyl), —O(CH₂CH₂)ₛ—OR¹¹, —C(R¹²)₂—O—(CH₂CH₂)ₛ—OR¹¹, and 5-6 membered heteroaryl; wherein R¹ may optionally be substituted by one or more substituents each selected from R⁵; R¹⁰ is hydrogen or deuterium; R¹¹ and R¹² are each independently hydrogen or C₁-C₃alkyl optionally substituted by one or more halogens; R² is selected from the group consisting of C₁-C₆alkyl, CH₂-R²², and C₃-C₆cycloalkyl; wherein R² or R²² may optionally be substituted by one, two, or three substituents each selected from R⁵; wherein when R¹ is —CF₃, R² is not t-butyl; R²² is selected from the group consisting of phenyl, naphthyl, and 8-10 membered bicyclic heteroaryl; R^{3a} is hydrogen or C₁-C₃alkyl; R³ is selected from the group consisting of C₁-C₆alkyl, C₃-C₆cycloalkyl, phenyl and 4-6 heterocyclyl and 5-6 membered heteroaryl; wherein R³ may optionally be substituted by one, two, or three substituents each selected from R⁵; or R³ is —Si(CH₃)₃; or R³⁰ and R³, together with the atoms to which they are attached, may be joined together to form a 4-6 membered monocyclic, 6-10 membered fused bicyclic, or 6-10 membered spirocyclic heterocycle which may optionally be substituted by one, two or three substituents each selected from R⁵; R⁴ is

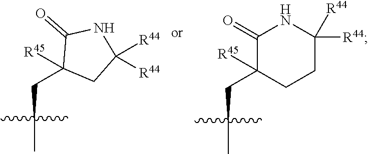

R⁴⁴ is each independently selected from the group consisting of hydrogen, —CH₃ and —CF₃, or two R⁴⁴ groups, together with the carbon to which they are attached, may be joined together to form a cyclopropyl, cyclobutyl, or oxetanyl; R⁴⁵ is hydrogen; or one R⁴⁴ and R⁴⁵, together with the atoms to which they are attached, may be joined together to form a 4-6 membered carbocycle; R⁵ is independently selected for each occurrence from the group consisting of halogen, hydroxyl, cyano, —N(R⁵⁵)₂, —N(R⁵⁵)C(O)R⁵⁵, —C(O)N(R⁵⁵)₂, —OPO(OH)₂, —CO₂H, —SO₂CH₃ —CF₃, —CHF₂, C₁-C₆alkyl, C₁-C₆alkoxy, and phenyl; wherein C₁-C₆alkyl, C₁-C₆alkoxy and phenyl may optionally be substituted by one or more substituents each independently selected from the group consisting of halogen and hydroxyl; and wherein two geminal R⁵ groups, together with the carbon to which they are attached, may be joined together to form a cyclopropyl or cyclobutyl; and R⁵⁵ is hydrogen or C₁-C₃alkyl optionally substituted by one, two or three halogens; or two R⁵⁵ groups, together with the atoms to which they are attached, may be joined together to form a 4-6 membered heterocyclyl, wherein the heterocycicyl may optionally be substituted by one, two or three substituents each independently selected from halogen, hydroxyl and C₁-C₃alkyl (optionally substituted by one or more halogens).

In some embodiments, disclosed herein is a protease inhibitory compound represented by Formula I:

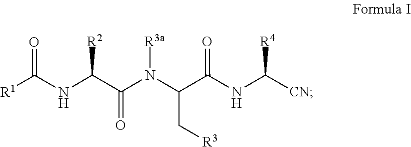

or a pharmaceutically acceptable salt or stereoisomer thereof, wherein R¹ is selected from the group consisting of —CF₃, t-butyl, —O—C₆-C₁°cycloalkyl, —O—C(R¹⁰)₂-C₆-C₁₀cycloalkyl, —O—C(R¹⁰)₂-phenyl, —O(CH₂CH₂)ₛ—OR¹¹, —C(R¹²)₂—O—(CH₂CH₂)₅—OR¹¹, and 5-6 membered heteroaryl; wherein R¹ may optionally be substituted by one, two, or three substituents each selected from R⁵; R¹⁰ is hydrogen or deuterium; R² is C₁-C₆alkyl or CH₂-R²²; wherein R² or R²² may optionally be substituted by one, two, or three substituents each selected from R⁵; wherein when R¹ is —CF₃. R² is not t-butyl; R²² is selected from the group consisting of phenyl, naphthyl, and 8-10 membered bicyclic heteroaryl; R^{3a} is hydrogen or C₁-C₃alkyl; R³ is selected from the group consisting of C₁-C₆alkyl, C₃-C₆cycloalkyl, and 4-6 membered heterocyclyl; wherein R³ may optionally be substituted by one, two, or three substituents each selected from R⁵; or R³ is —Si(CH₃)₃; or R^{3a} and R³, together with the atoms to which they are attached, may be joined together to form a 4-6 membered monocyclic, 6-10 membered fused bicyclic, or 6-10 membered spirocyclic heterocycle which may optionally be substituted by one, two or three substituents each selected from R⁵; R⁴ is

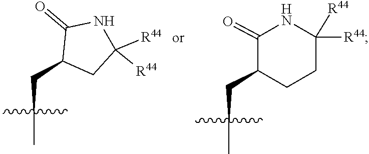

R⁴⁴ is each independently hydrogen or methyl; or two R⁴⁴ groups, together with the carbon to which they are attached, may be joined together to form a cyclopropyl, cyclobutyl, or oxetanyl; R⁵ is independently selected for each occurrence from the group consisting of halogen, hydroxyl, cyano, —N(R⁵⁵)₂, —N(R⁵⁵)C(O)R⁵⁵, —C(O)N(R⁵⁵)₂, —CF₃, C₁-C₆alkyl, C₁-C₆alkoxy, and phenyl; wherein C₁-C₆alkyl, C₁-C₆alkoxy and phenyl may optionally be substituted by one or more substituents each independently selected from the group consisting of halogen and hydroxyl; and R⁵⁵ is hydrogen or C₁-C₃alkyl optionally substituted by one, two or three halogens.

In some embodiments, R¹ is selected from the group consisting of —CF₃, t-butyl, —O—C₆-C₁₀cycloalkyl, —O—C(R¹⁰)₂-C₆-C₁₀cycloalkyl, —O—C(R¹⁰)₂-phenyl, —O(CH₂CH₂)ₛ—OR¹¹, —C(R¹²)₂—O—(CH₂CH₂)ₛ—OR¹¹, and 5-6 membered heteroaryl; wherein R¹ may optionally be substituted by one or more substituents each selected from R^{s}.

For example, in certain embodiments, R¹ is selected from the group consisting of:

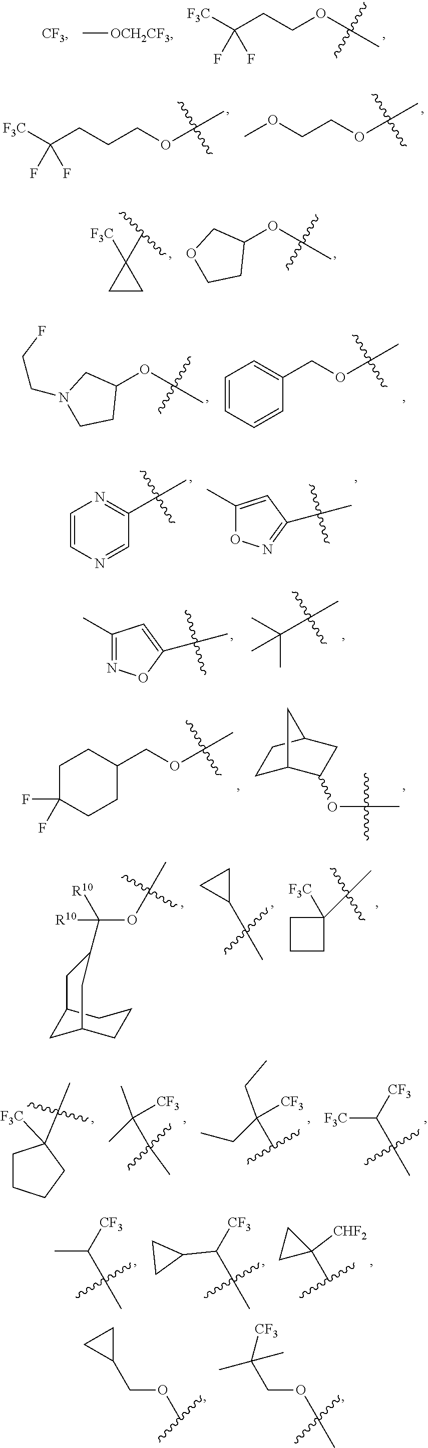

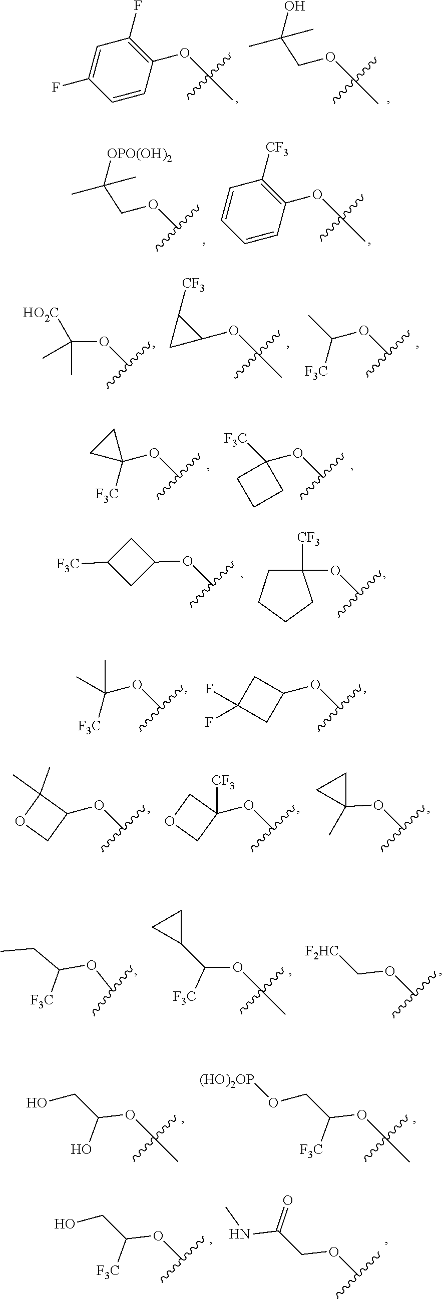

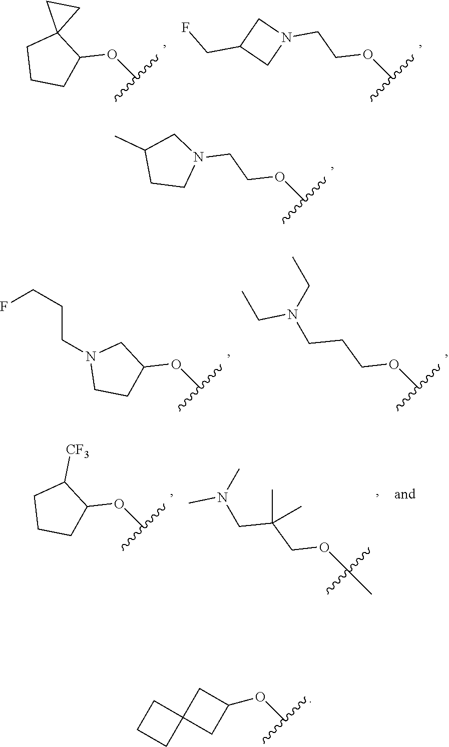

In further embodiments, R² is C₁-C₆alkyl or CH₂-R²²; wherein R² or R²² may optionally be substituted by one, two, or three substituents each selected from R⁵. For example, in some embodiments R¹ may be selected from the group consisting of:

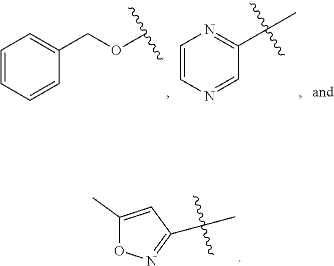

In other embodiments, K is selected from the group consisting of:

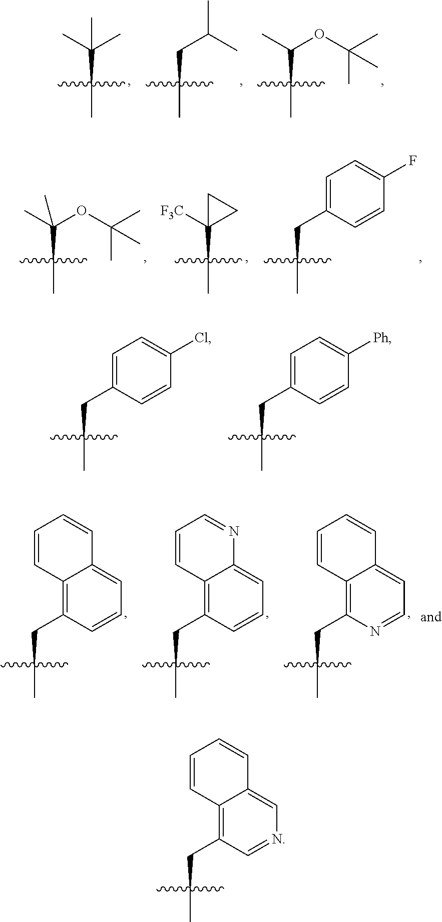

For example, R² may be selected from the group consisting of:

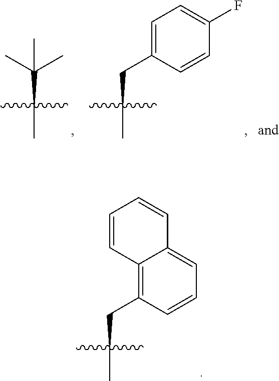

In some embodiments, R² is selected from the group consisting of

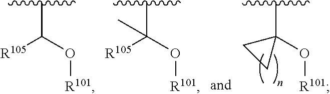

wherein R¹⁰⁵ is C₁-C₆alkyl, and n is 1, 2, or 3; R¹⁰¹ is selected from the group consisting of C₁-C₆alkyl, C₂-C₆alkenyl, C₃-C₆cycloalkyl, and C₃-C₆cycloalkenyl, wherein R¹⁰¹ may optionally be substituted by one, two, three, or four substituents each independently selected from R^{D}; R^{D} is selected from the group consisting of halogen, C₁-C₆alkyl, C₁-C₆alkoxy, C₃-C₆cycloalkyl, and cyano, wherein the alkyl may optionally be substituted by one or more halo.

In some embodiments, R² is selected from the group consisting of

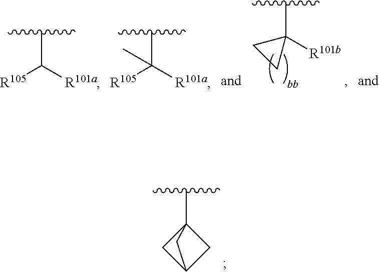

wherein R^{105a} is C₁-C₂alkyl, bb is 1 or 2, R^{101a} and R^{101b} is each independently selected from the group consisting of C₁-C₆alkyl, C₂-C₆alkenyl, C₃-C₆cycloalkyl, and C₃-C₆cycloalkenyl, wherein R^{101a} or R^{101b} may optionally be substituted by one, two, three, or four substituents each independently selected from R^{D}; R^{D} is selected from the group consisting of halogen, C₁-C₆alkyl, C₁-C₆alkoxy, C₃-C₆cycloalkyl, and cyano, wherein the alkyl may optionally be substituted by one or more halo.

In some embodiments, R² is selected from the group consisting of

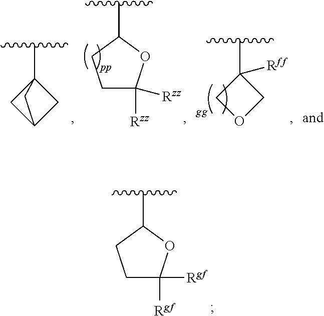

wherein pp is selected from 0, 1, 2 and 3, R^{zz} is each independently H or C₁-C₆alkyl(e.g., CH₃), gg is 1, 2 or 3, R^{ff} is H or C₁-C₆alkyl (e.g., CH₃), and R^{gf} is each independently H or C₁-C₆alkyl (e.g., CH₃).

In some embodiments, R⁻³′ is hydrogen. In other embodiments, R³ is selected from the group consisting of:

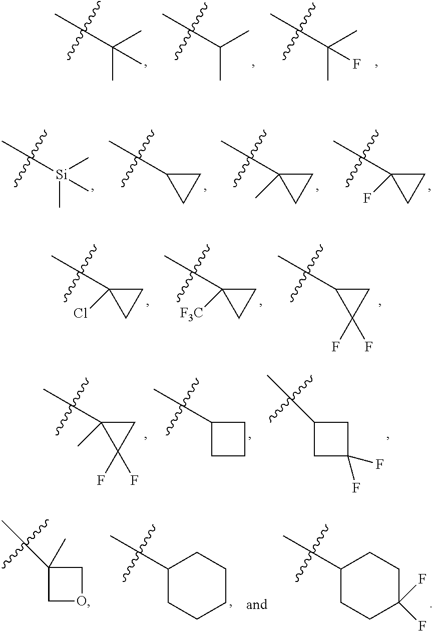

For example, R³ may selected from the group consisting of:

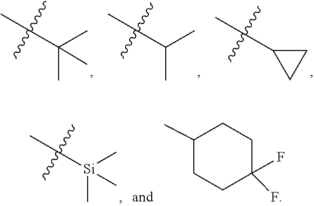

In other embodiments, R^{3a} and R³, together with the atoms to which they are attached, join together to form a 4-6 membered monocyclic, 6-10 membered fused bicyclic, or 6-10 membered spirocyclic heterocycle.

For example, in some embodiments a disclosed compound is represented by:

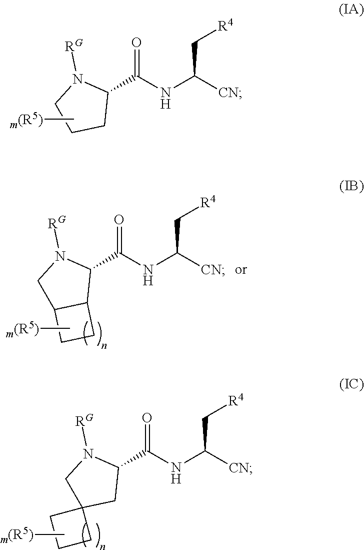

wherein m is selected from 0, 1, 2 and 3; n is selected from 0, 1, 2 and 3; and R^{G} is

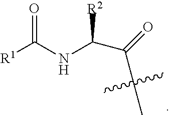

For example, in some embodiments a disclosed compound is represented by:

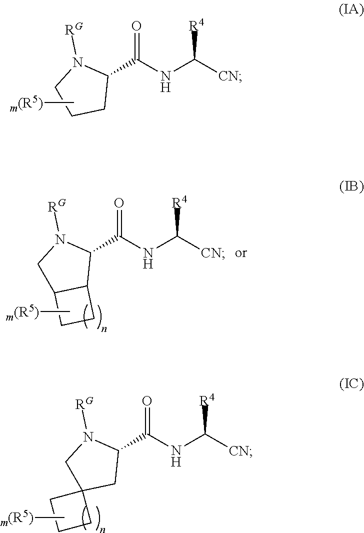

wherein m is selected from 0, 1, 2 and 3; n is selected from 0, 1, 2 and 3; and R^{G} is

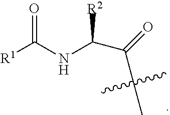

In some embodiments, R^{3a} and R³, together with the atoms to which they are attached, join together to form a 4-6 membered monocyclic, 6-10 membered fused bicyclic, or 6-10 membered spirocyclic selected from the group consisting of:

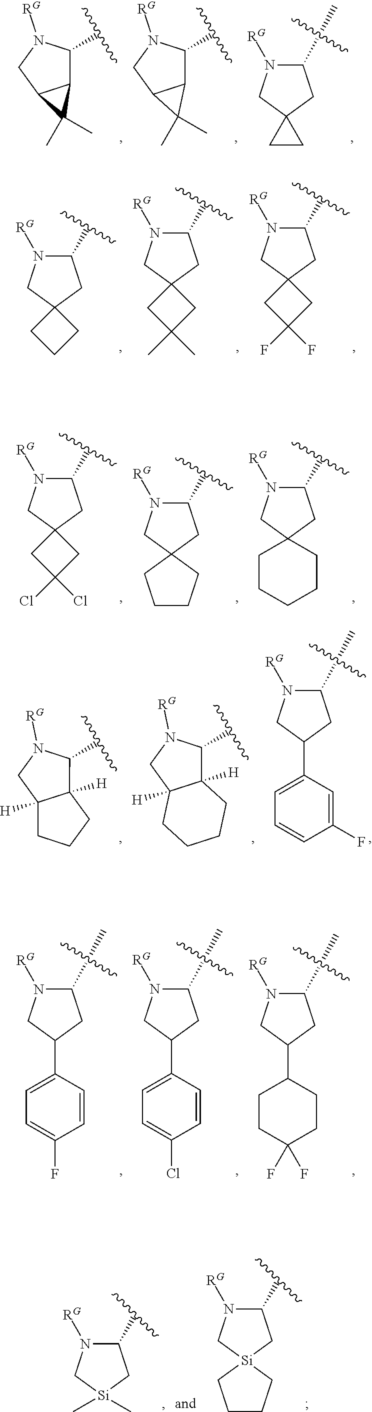

wherein R^{G} is

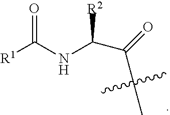

In certain embodiments, R⁴ is selected from the group consisting of:

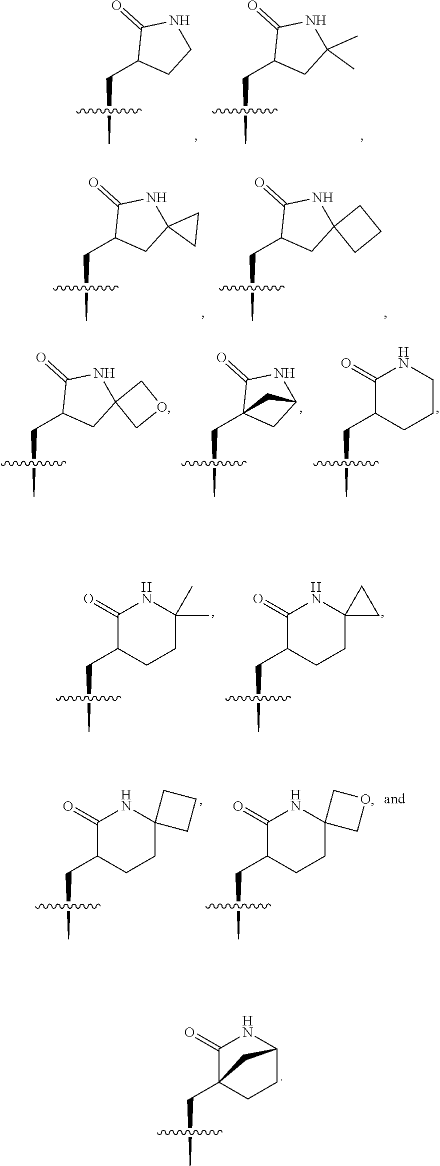

In certain embodiments, R⁴ is selected from the group consisting of:

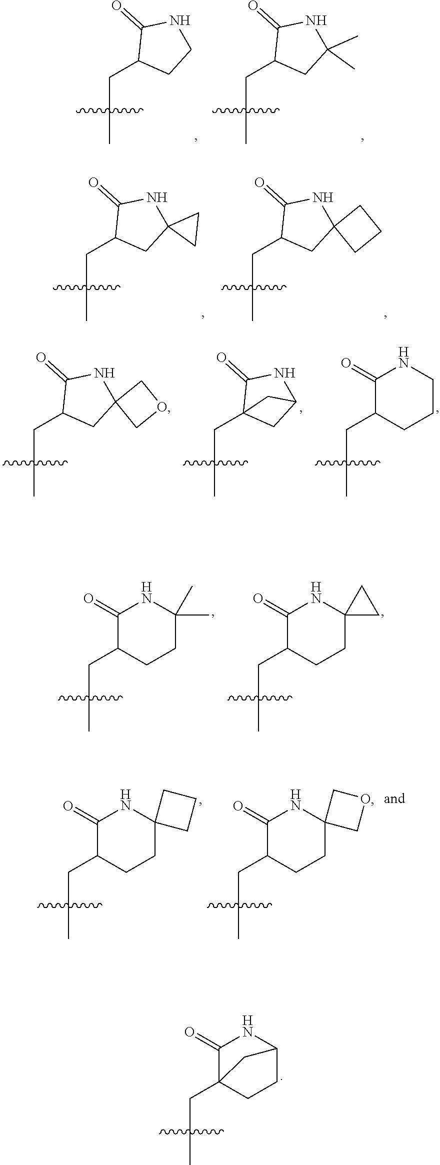

In further embodiments, R⁵ is selected from the group consisting of chloro, fluoro, —CF₃, —CH₃ and phenyl.

In further embodiments, R¹ is a 7-8 membered spirocyclic heterocyclyl.

In further embodiments, R² is selected from the group consisting of C₁-C₆alkyl, CH₂-C₃-C₆cycloalkyl, C₃-C₆cycloalkyl, and 5-6 membered heterocyclyl; wherein R² may optionally be substituted by one, two, or three substituents each selected from R^{s}.

In further embodiments, R⁴ is selected from the group consisting of

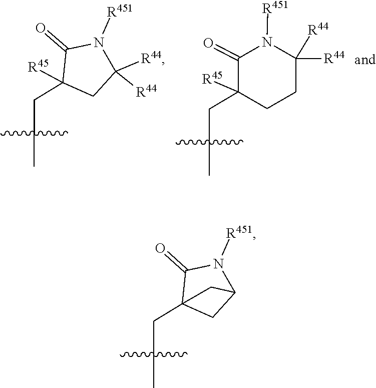

wherein R⁴⁴ is each independently selected from the group consisting of hydrogen, —CH₃ and —CF₃, or two R⁴⁴ groups, together with the carbon to which they are attached, may be joined together to form a cyclopropyl, cyclobutyl, or oxetanyl; and R⁴⁵¹ is hydrogen or C₁₋₃ alkyl.

In further embodiments, R⁴ is selected from the group consisting of

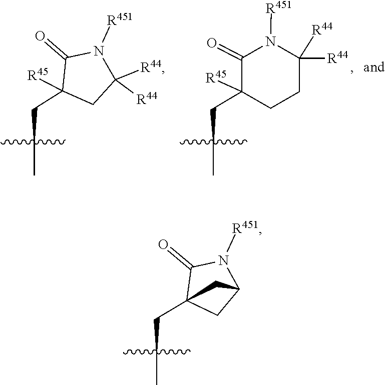

wherein R⁴⁴ is each independently selected from the group consisting of hydrogen, —CH₃ and —CF₃, or two R⁴⁴ groups, together with the carbon to which they are attached, may be joined together to form a cyclopropyl, cyclobutyl, or oxetanyl; and R^{45′} is hydrogen or C₁₋₃ alkyl.

In further embodiments, R⁵ is independently selected for each occurrence from the group consisting of halogen, hydroxyl, cyano, —N(R⁵⁵)₂, —N(R⁵⁵)C(O)R⁵⁵, —C(O)N(R⁵⁵)₂, —OPO(OH)₂, —CO₂H, —SO₂CH₃ —CF₃, —CHF₂, C₁-C₆alkyl, C₁-C₆alkoxy, phenyl, —O-phenyl, —O—(C₃-C₆cycloalkyl), —O-(5-6 membered heterocyclyl) and —O-(5-6 membered heteroaryl); wherein C₁-C₆alkyl, C₁-C₆alkoxy and phenyl may optionally be substituted by one or more substituents each independently selected from the group consisting of halogen and hydroxyl; wherein the cycloalkyl and heterocyclyl may optionally be substituted by one or more substituents each independently selected from the group consisting of halogen, —CF₃, —CHF₂, cyano, C₁-C₆alkyl and C₃-C₆cycloalkyl; and wherein two geminal R⁵ groups, together with the carbon to which they are attached, may be joined together to form a cyclopropyl or cyclobutyl; and R⁵⁵ is hydrogen or C₁-C₃alkyl optionally substituted by one, two or three halogens; or two R⁵⁵ groups, together with the atoms to which they are attached, may be joined together to form a 4-6 membered heterocyclyl, wherein the heterocycicyl may optionally be substituted by one, two or three substituents each independently selected from halogen, hydroxyl and C₁-C₃alkyl(optionally substituted by one or more halogens).

In some embodiments, a disclosed compound is selected from the group consisting of the compounds identified in Table 1, or pharmaceutically acceptable salt thereof.

TABLE 1

| Cmpd | |
|---|---|
| No. | Structure |
| 101 | 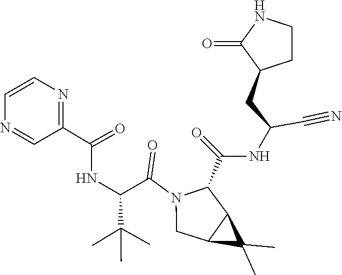 [SMILES: CC(C)(C)[C@H](NC(=O)c1cnccn1)C(=O)N1C[C@H]2[C@@H]([C@H]1C(=O)N[C@H](C#N)C[C@@H]1CCNC1=O)C2(C)C] |
| 102 | 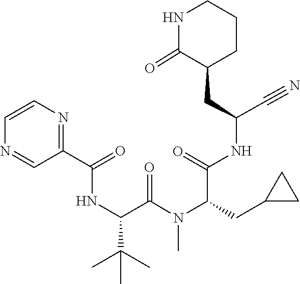 [SMILES: CN(C(=O)[C@@H](NC(=O)c1cnccn1)C(C)(C)C)[C@@H](CC1CC1)C(=O)N[C@H](C#N)C[C@@H]1CCCNC1=O] |
| 103 | 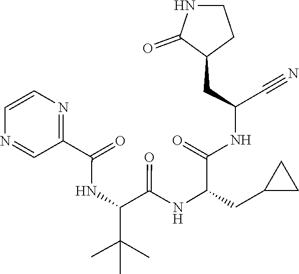 [SMILES: CC(C)(C)[C@H](NC(=O)c1cnccn1)C(=O)N[C@@H](CC1CC1)C(=O)N[C@H](C#N)C[C@@H]1CCNC1=O] |
| 104 | 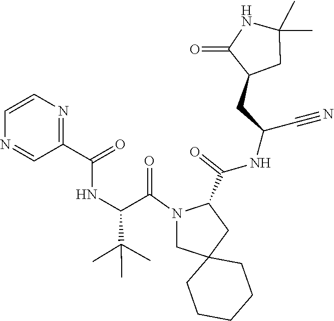 [SMILES: CC1(C)C[C@@H](C[C@@H](C#N)NC(=O)[C@@H]2CC3(CCCCC3)CN2C(=O)[C@@H](NC(=O)c2cnccn2)C(C)(C)C)C(=O)N1] |
| 105 | 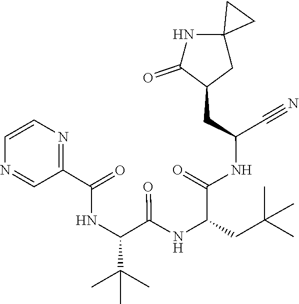 [SMILES: CC(C)(C)C[C@H](NC(=O)[C@@H](NC(=O)c1cnccn1)C(C)(C)C)C(=O)N[C@H](C#N)C[C@@H]1CC2(CC2)NC1=O] |
| 106 | 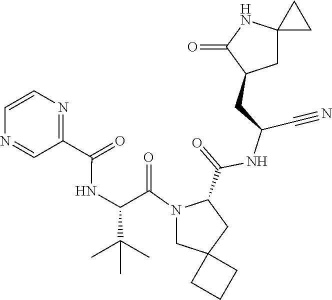 [SMILES: CC(C)(C)[C@H](NC(=O)c1cnccn1)C(=O)N1CC2(CCC2)C[C@H]1C(=O)N[C@H](C#N)C[C@@H]1CC2(CC2)NC1=O] |
| 107 | 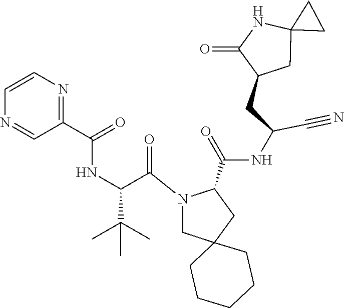 [SMILES: CC(C)(C)[C@H](NC(=O)c1cnccn1)C(=O)N1CC2(CCCCC2)C[C@H]1C(=O)N[C@H](C#N)C[C@@H]1CC2(CC2)NC1=O] |
| 108 | 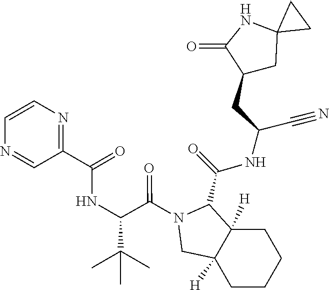 |
| 109 | 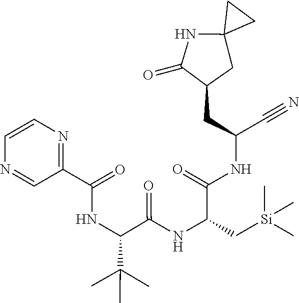 [SMILES: CC(C)(C)[C@H](NC(=O)c1cnccn1)C(=O)N[C@@H](C[Si](C)(C)C)C(=O)N[C@H](C#N)C[C@@H]1CC2(CC2)NC1=O] |
| 110 | 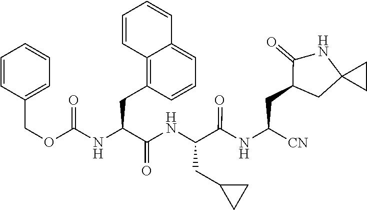 [SMILES: N#C[C@H](C[C@@H]1CC2(CC2)NC1=O)NC(=O)[C@H](CC1CC1)NC(=O)[C@H](Cc1cccc2ccccc12)NC(=O)OCc1ccccc1] |
| 111 | 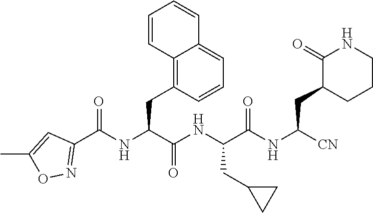 [SMILES: Cc1cc(C(=O)N[C@@H](Cc2cccc3ccccc23)C(=O)N[C@@H](CC2CC2)C(=O)N[C@H](C#N)C[C@@H]2CCCNC2=O)no1] |
| 112 | 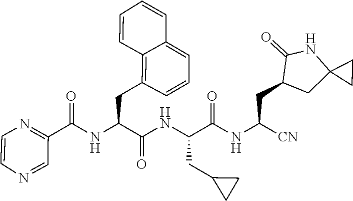 [SMILES: N#C[C@H](C[C@@H]1CC2(CC2)NC1=O)NC(=O)[C@H](CC1CC1)NC(=O)[C@H](Cc1cccc2ccccc12)NC(=O)c1cnccn1] |
| 113 | 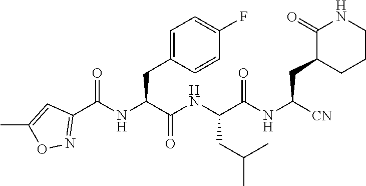 [SMILES: Cc1cc(C(=O)N[C@@H](Cc2ccc(F)cc2)C(=O)N[C@@H](CC(C)C)C(=O)N[C@H](C#N)C[C@@H]2CCCNC2=O)no1] |
| 114 | 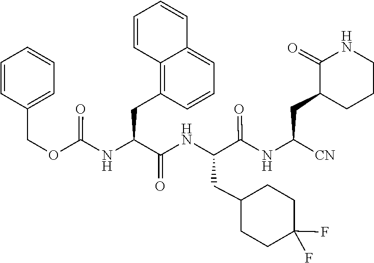 [SMILES: N#C[C@H](C[C@@H]1CCCNC1=O)NC(=O)[C@H](CC1CCC(F)(F)CC1)NC(=O)[C@H](Cc1cccc2ccccc12)NC(=O)OCc1ccccc1] |
| 115 | 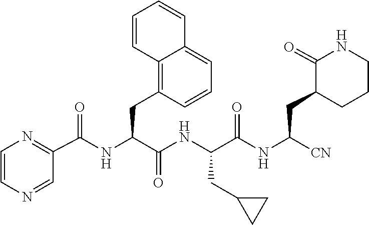 [SMILES: N#C[C@H](C[C@@H]1CCCNC1=O)NC(=O)[C@H](CC1CC1)NC(=O)[C@H](Cc1cccc2ccccc12)NC(=O)c1cnccn1] |
| 116 | 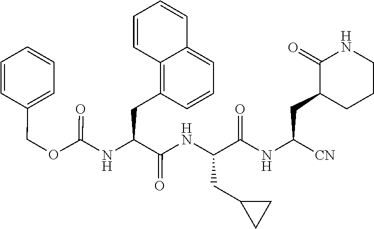 [SMILES: N#C[C@H](C[C@@H]1CCCNC1=O)NC(=O)[C@H](CC1CC1)NC(=O)[C@H](Cc1cccc2ccccc12)NC(=O)OCc1ccccc1] |
| 117 | 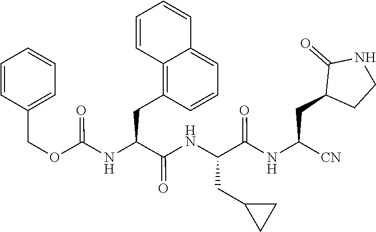 [SMILES: N#C[C@H](C[C@@H]1CCNC1=O)NC(=O)[C@H](CC1CC1)NC(=O)[C@H](Cc1cccc2ccccc12)NC(=O)OCc1ccccc1] |
| 118 | 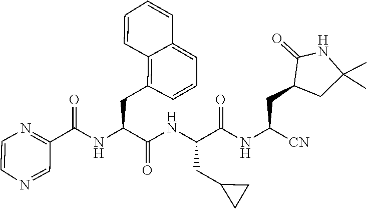 [SMILES: CC1(C)C[C@@H](C[C@@H](C#N)NC(=O)[C@H](CC2CC2)NC(=O)[C@H](Cc2cccc3ccccc23)NC(=O)c2cnccn2)C(=O)N1] |
| 119 | 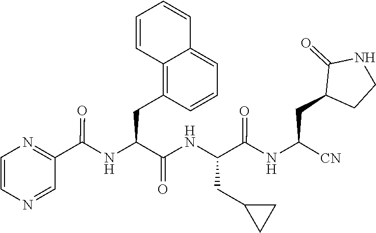 [SMILES: N#C[C@H](C[C@@H]1CCNC1=O)NC(=O)[C@H](CC1CC1)NC(=O)[C@H](Cc1cccc2ccccc12)NC(=O)c1cnccn1] |
| 120 | 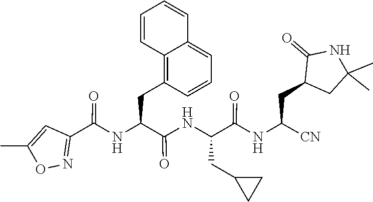 |
| 121 | 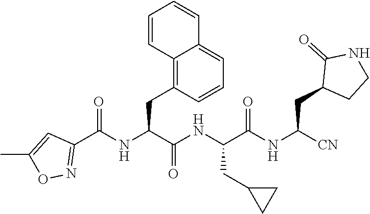 [SMILES: Cc1cc(C(=O)N[C@@H](Cc2cccc3ccccc23)C(=O)N[C@@H](CC2CC2)C(=O)N[C@H](C#N)C[C@@H]2CCNC2=O)no1] |
| 122 | 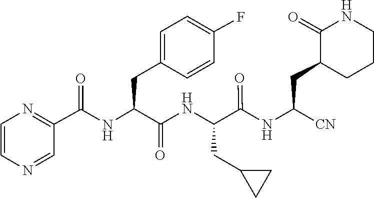 [SMILES: N#C[C@H](C[C@@H]1CCCNC1=O)NC(=O)[C@H](CC1CC1)NC(=O)[C@H](Cc1ccc(F)cc1)NC(=O)c1cnccn1] |
| 123 | 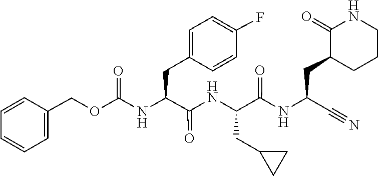 [SMILES: N#C[C@H](C[C@@H]1CCCNC1=O)NC(=O)[C@H](CC1CC1)NC(=O)[C@H](Cc1ccc(F)cc1)NC(=O)OCc1ccccc1] |
| 124 | 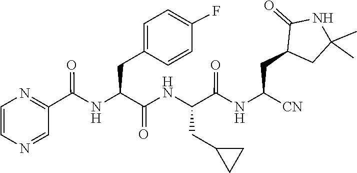 [SMILES: CC1(C)C[C@@H](C[C@@H](C#N)NC(=O)[C@H](CC2CC2)NC(=O)[C@H](Cc2ccc(F)cc2)NC(=O)c2cnccn2)C(=O)N1] |
| 125 | 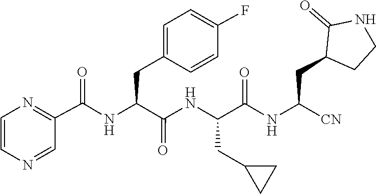 [SMILES: N#C[C@H](C[C@@H]1CCNC1=O)NC(=O)[C@H](CC1CC1)NC(=O)[C@H](Cc1ccc(F)cc1)NC(=O)c1cnccn1] |
| 126 | 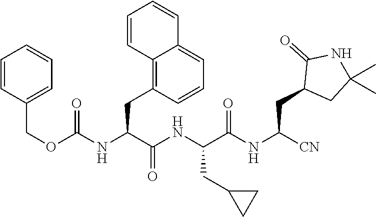 [SMILES: CC1(C)C[C@@H](C[C@@H](C#N)NC(=O)[C@H](CC2CC2)NC(=O)[C@H](Cc2cccc3ccccc23)NC(=O)OCc2ccccc2)C(=O)N1] |
| 127 | 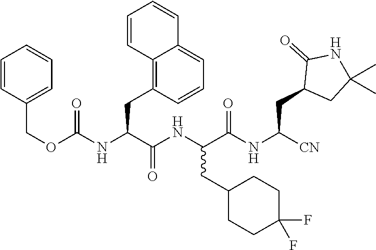 [SMILES: CC1(C)C[C@@H](C[C@@H](C#N)NC(=O)C(CC2CCC(F)(F)CC2)NC(=O)[C@H](Cc2cccc3ccccc23)NC(=O)OCc2ccccc2)C(=O)N1] |
| 128 | 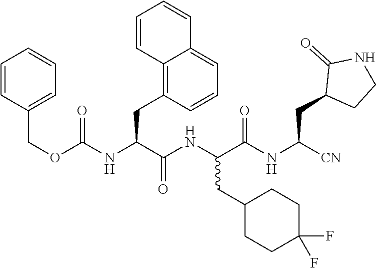 [SMILES: N#C[C@H](C[C@@H]1CCNC1=O)NC(=O)C(CC1CCC(F)(F)CC1)NC(=O)[C@H](Cc1cccc2ccccc12)NC(=O)OCc1ccccc1] |
| 129 | 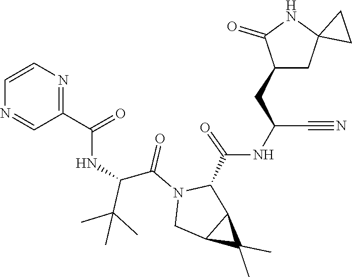 [SMILES: CC(C)(C)[C@H](NC(=O)c1cnccn1)C(=O)N1C[C@H]2[C@@H]([C@H]1C(=O)N[C@H](C#N)C[C@@H]1CC3(CC3)NC1=O)C2(C)C] |
| 130 | 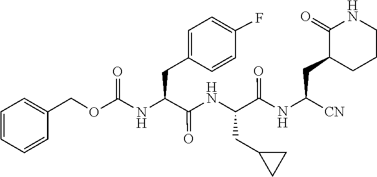 [SMILES: N#C[C@H](C[C@@H]1CCCNC1=O)NC(=O)[C@H](CC1CC1)NC(=O)[C@H](Cc1ccc(F)cc1)NC(=O)OCc1ccccc1] |
| 131 | 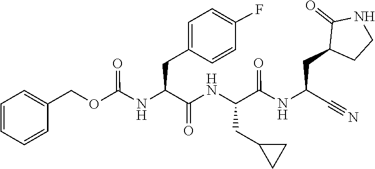 [SMILES: N#C[C@H](C[C@@H]1CCNC1=O)NC(=O)[C@H](CC1CC1)NC(=O)[C@H](Cc1ccc(F)cc1)NC(=O)OCc1ccccc1] |
| 132 | 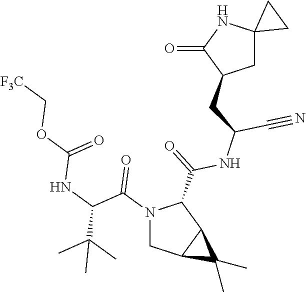 [SMILES: CC(C)(C)[C@H](NC(=O)OCC(F)(F)F)C(=O)N1C[C@H]2[C@@H]([C@H]1C(=O)N[C@H](C#N)C[C@@H]1CC3(CC3)NC1=O)C2(C)C] |
| 133 | 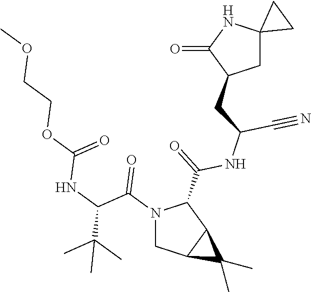 [SMILES: COCCOC(=O)N[C@H](C(=O)N1C[C@H]2[C@@H]([C@H]1C(=O)N[C@H](C#N)C[C@@H]1CC3(CC3)NC1=O)C2(C)C)C(C)(C)C] |
| 134 | 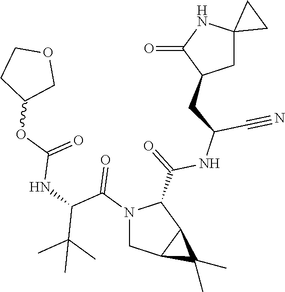 [SMILES: CC(C)(C)[C@H](NC(=O)OC1CCOC1)C(=O)N1C[C@H]2[C@@H]([C@H]1C(=O)N[C@H](C#N)C[C@@H]1CC3(CC3)NC1=O)C2(C)C] |
| 135 | 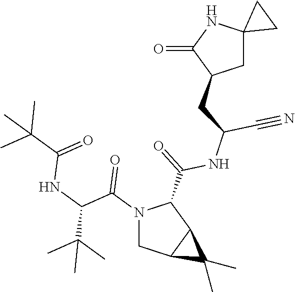 [SMILES: CC(C)(C)C(=O)N[C@H](C(=O)N1C[C@H]2[C@@H]([C@H]1C(=O)N[C@H](C#N)C[C@@H]1CC3(CC3)NC1=O)C2(C)C)C(C)(C)C] |
| 136 | 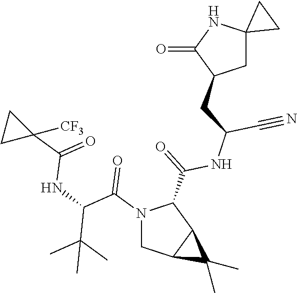 [SMILES: CC(C)(C)[C@H](NC(=O)C1(C(F)(F)F)CC1)C(=O)N1C[C@H]2[C@@H]([C@H]1C(=O)N[C@H](C#N)C[C@@H]1CC3(CC3)NC1=O)C2(C)C] |
| 137 | 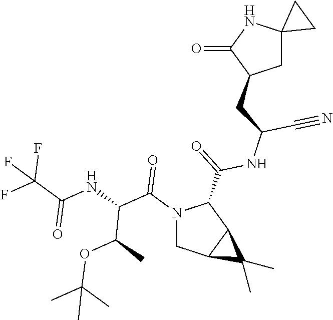 [SMILES: C[C@@H](OC(C)(C)C)[C@H](NC(=O)C(F)(F)F)C(=O)N1C[C@H]2[C@@H]([C@H]1C(=O)N[C@H](C#N)C[C@@H]1CC3(CC3)NC1=O)C2(C)C] |
| 138 | 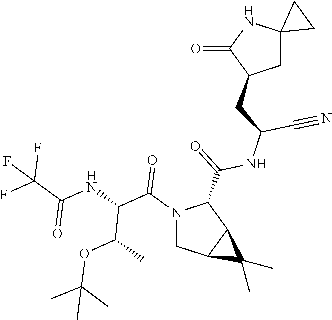 [SMILES: C[C@H](OC(C)(C)C)[C@H](NC(=O)C(F)(F)F)C(=O)N1C[C@H]2[C@@H]([C@H]1C(=O)N[C@H](C#N)C[C@@H]1CC3(CC3)NC1=O)C2(C)C] |
| 139 | 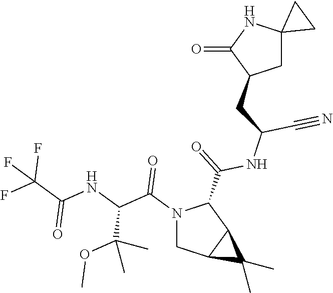 [SMILES: COC(C)(C)[C@H](NC(=O)C(F)(F)F)C(=O)N1C[C@H]2[C@@H]([C@H]1C(=O)N[C@H](C#N)C[C@@H]1CC3(CC3)NC1=O)C2(C)C] |
| 140 | 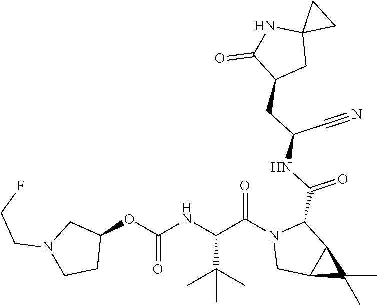 [SMILES: CC(C)(C)[C@H](NC(=O)O[C@H]1CCN(CCF)C1)C(=O)N1C[C@H]2[C@@H]([C@H]1C(=O)N[C@H](C#N)C[C@@H]1CC3(CC3)NC1=O)C2(C)C] |
| 141 | 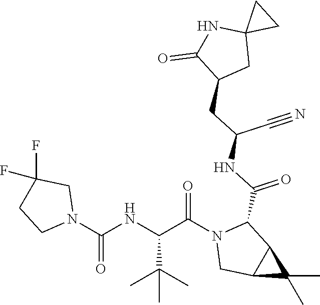 [SMILES: CC(C)(C)[C@H](NC(=O)N1CCC(F)(F)C1)C(=O)N1C[C@H]2[C@@H]([C@H]1C(=O)N[C@H](C#N)C[C@@H]1CC3(CC3)NC1=O)C2(C)C] |
| 142 | 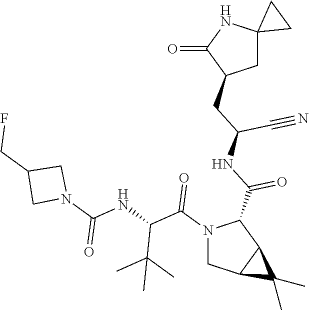 [SMILES: CC(C)(C)[C@H](NC(=O)N1CC(CF)C1)C(=O)N1C[C@H]2[C@@H]([C@H]1C(=O)N[C@H](C#N)C[C@@H]1CC3(CC3)NC1=O)C2(C)C] |
| 143 | 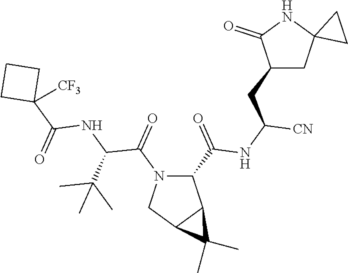 [SMILES: CC(C)(C)[C@H](NC(=O)C1(C(F)(F)F)CCC1)C(=O)N1C[C@H]2[C@@H]([C@H]1C(=O)N[C@H](C#N)C[C@@H]1CC3(CC3)NC1=O)C2(C)C] |
| 144 | 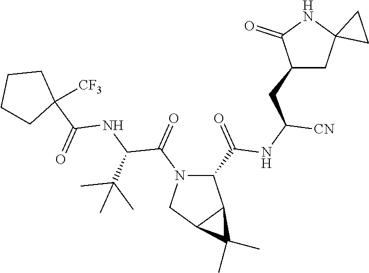 [SMILES: CC(C)(C)[C@H](NC(=O)C1(C(F)(F)F)CCCC1)C(=O)N1C[C@H]2[C@@H]([C@H]1C(=O)N[C@H](C#N)C[C@@H]1CC3(CC3)NC1=O)C2(C)C] |
| 145 | 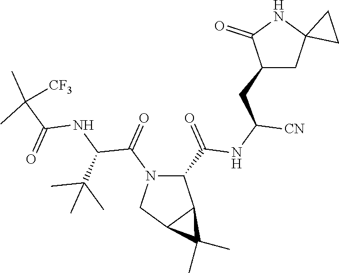 [SMILES: CC(C)(C)[C@H](NC(=O)C(C)(C)C(F)(F)F)C(=O)N1C[C@H]2[C@@H]([C@H]1C(=O)N[C@H](C#N)C[C@@H]1CC3(CC3)NC1=O)C2(C)C] |
| 146 | 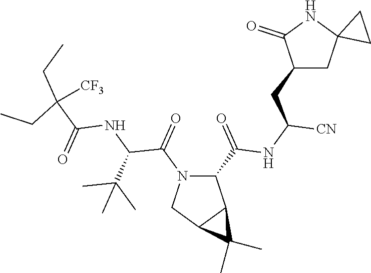 [SMILES: CCC(CC)(C(=O)N[C@H](C(=O)N1C[C@H]2[C@@H]([C@H]1C(=O)N[C@H](C#N)C[C@@H]1CC3(CC3)NC1=O)C2(C)C)C(C)(C)C)C(F)(F)F] |
| 147 | 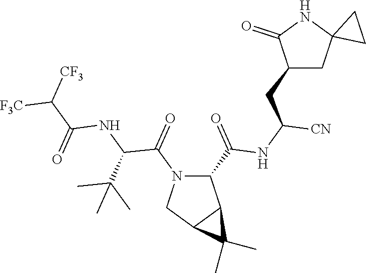 [SMILES: CC(C)(C)[C@H](NC(=O)C(C(F)(F)F)C(F)(F)F)C(=O)N1C[C@H]2[C@@H]([C@H]1C(=O)N[C@H](C#N)C[C@@H]1CC3(CC3)NC1=O)C2(C)C] |
| 148 | 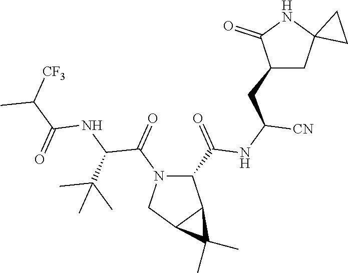 [SMILES: CC(C(=O)N[C@H](C(=O)N1C[C@H]2[C@@H]([C@H]1C(=O)N[C@H](C#N)C[C@@H]1CC3(CC3)NC1=O)C2(C)C)C(C)(C)C)C(F)(F)F] |
| 149 | 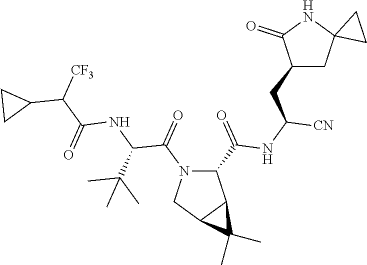 [SMILES: CC(C)(C)[C@H](NC(=O)C(C1CC1)C(F)(F)F)C(=O)N1C[C@H]2[C@@H]([C@H]1C(=O)N[C@H](C#N)C[C@@H]1CC3(CC3)NC1=O)C2(C)C] |
| 150 | 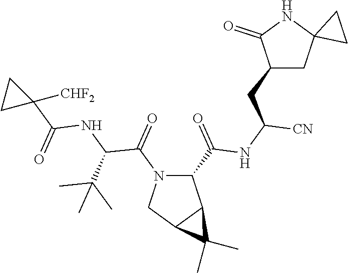 [SMILES: CC(C)(C)[C@H](NC(=O)C1(C(F)F)CC1)C(=O)N1C[C@H]2[C@@H]([C@H]1C(=O)N[C@H](C#N)C[C@@H]1CC3(CC3)NC1=O)C2(C)C] |
| 151 | 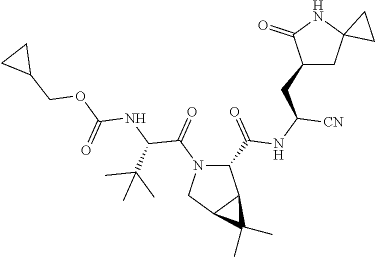 [SMILES: CC(C)(C)[C@H](NC(=O)OCC1CC1)C(=O)N1C[C@H]2[C@@H]([C@H]1C(=O)N[C@H](C#N)C[C@@H]1CC3(CC3)NC1=O)C2(C)C] |
| 152 | 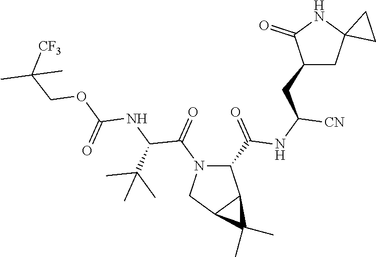 [SMILES: CC(C)(C)[C@H](NC(=O)OCC(C)(C)C(F)(F)F)C(=O)N1C[C@H]2[C@@H]([C@H]1C(=O)N[C@H](C#N)C[C@@H]1CC3(CC3)NC1=O)C2(C)C] |
| 153 | 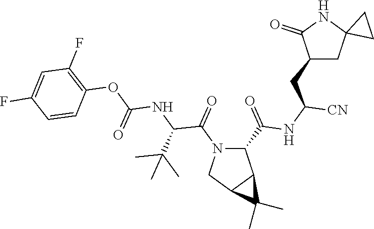 [SMILES: CC(C)(C)[C@H](NC(=O)Oc1ccc(F)cc1F)C(=O)N1C[C@H]2[C@@H]([C@H]1C(=O)N[C@H](C#N)C[C@@H]1CC3(CC3)NC1=O)C2(C)C] |
| 154 | 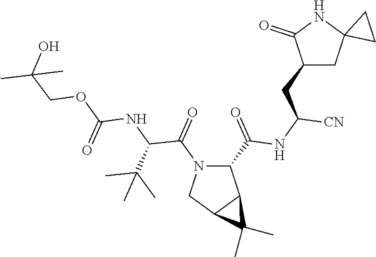 [SMILES: CC(C)(O)COC(=O)N[C@H](C(=O)N1C[C@H]2[C@@H]([C@H]1C(=O)N[C@H](C#N)C[C@@H]1CC3(CC3)NC1=O)C2(C)C)C(C)(C)C] |
| 155 | 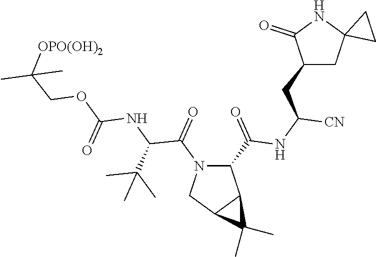 |
| 156 | 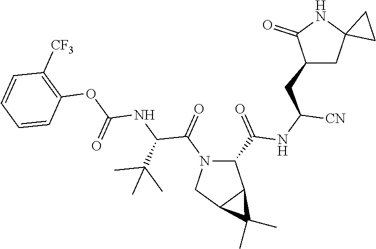 [SMILES: CC(C)(C)[C@H](NC(=O)Oc1ccccc1C(F)(F)F)C(=O)N1C[C@H]2[C@@H]([C@H]1C(=O)N[C@H](C#N)C[C@@H]1CC3(CC3)NC1=O)C2(C)C] |
| 157 | 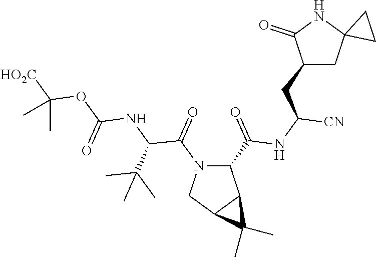 [SMILES: CC(C)(OC(=O)N[C@H](C(=O)N1C[C@H]2[C@@H]([C@H]1C(=O)N[C@H](C#N)C[C@@H]1CC3(CC3)NC1=O)C2(C)C)C(C)(C)C)C(=O)O] |
| 158 | 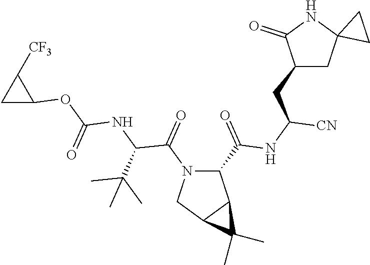 [SMILES: CC(C)(C)[C@H](NC(=O)OC1CC1C(F)(F)F)C(=O)N1C[C@H]2[C@@H]([C@H]1C(=O)N[C@H](C#N)C[C@@H]1CC3(CC3)NC1=O)C2(C)C] |
| 159 | 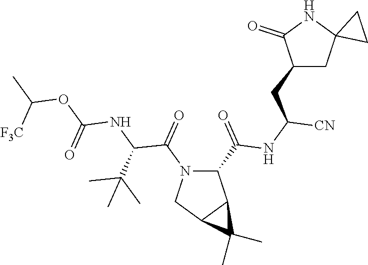 [SMILES: CC(OC(=O)N[C@H](C(=O)N1C[C@H]2[C@@H]([C@H]1C(=O)N[C@H](C#N)C[C@@H]1CC3(CC3)NC1=O)C2(C)C)C(C)(C)C)C(F)(F)F] |
| 160 | 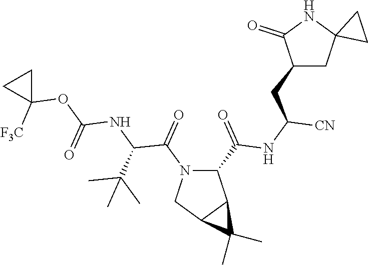 [SMILES: CC(C)(C)[C@H](NC(=O)OC1(C(F)(F)F)CC1)C(=O)N1C[C@H]2[C@@H]([C@H]1C(=O)N[C@H](C#N)C[C@@H]1CC3(CC3)NC1=O)C2(C)C] |
| 161 | 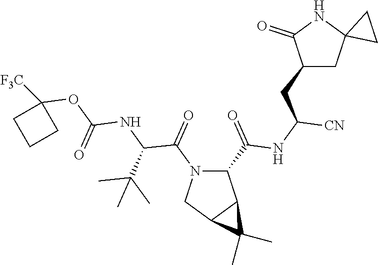 [SMILES: CC(C)(C)[C@H](NC(=O)OC1(C(F)(F)F)CCC1)C(=O)N1C[C@H]2[C@@H]([C@H]1C(=O)N[C@H](C#N)C[C@@H]1CC3(CC3)NC1=O)C2(C)C] |
| 162 | 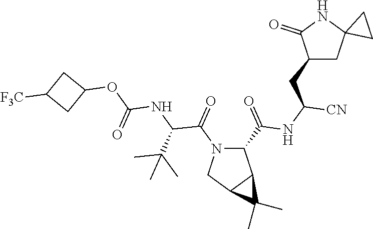 [SMILES: CC(C)(C)[C@H](NC(=O)OC1CC(C(F)(F)F)C1)C(=O)N1C[C@H]2[C@@H]([C@H]1C(=O)N[C@H](C#N)C[C@@H]1CC3(CC3)NC1=O)C2(C)C] |
| 163 | 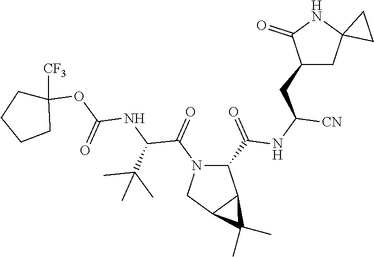 [SMILES: CC(C)(C)[C@H](NC(=O)OC1(C(F)(F)F)CCCC1)C(=O)N1C[C@H]2[C@@H]([C@H]1C(=O)N[C@H](C#N)C[C@@H]1CC3(CC3)NC1=O)C2(C)C] |
| 164 | 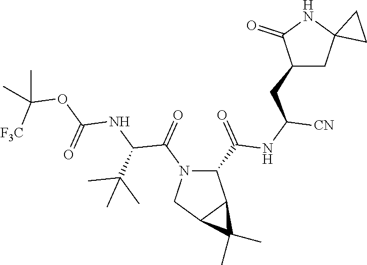 [SMILES: CC(C)(C)[C@H](NC(=O)OC(C)(C)C(F)(F)F)C(=O)N1C[C@H]2[C@@H]([C@H]1C(=O)N[C@H](C#N)C[C@@H]1CC3(CC3)NC1=O)C2(C)C] |
| 165 | 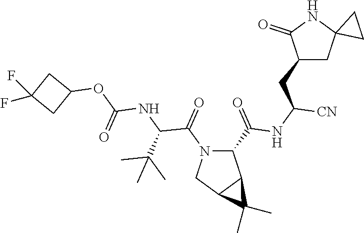 [SMILES: CC(C)(C)[C@H](NC(=O)OC1CC(F)(F)C1)C(=O)N1C[C@H]2[C@@H]([C@H]1C(=O)N[C@H](C#N)C[C@@H]1CC3(CC3)NC1=O)C2(C)C] |
| 166 | 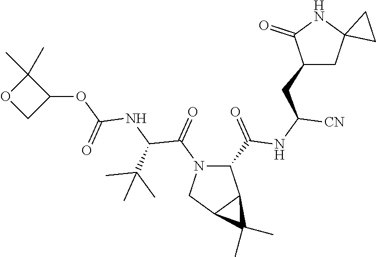 [SMILES: CC1(C)OCC1OC(=O)N[C@H](C(=O)N1C[C@H]2[C@@H]([C@H]1C(=O)N[C@H](C#N)C[C@@H]1CC3(CC3)NC1=O)C2(C)C)C(C)(C)C] |
| 167 | 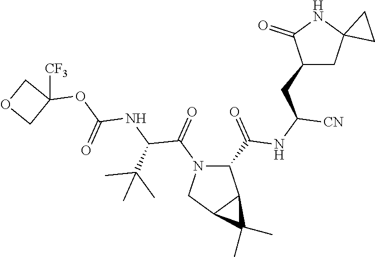 [SMILES: CC(C)(C)[C@H](NC(=O)OC1(C(F)(F)F)COC1)C(=O)N1C[C@H]2[C@@H]([C@H]1C(=O)N[C@H](C#N)C[C@@H]1CC3(CC3)NC1=O)C2(C)C] |
| 168 | 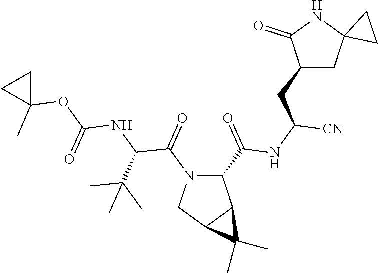 [SMILES: CC1(OC(=O)N[C@H](C(=O)N2C[C@H]3[C@@H]([C@H]2C(=O)N[C@H](C#N)C[C@@H]2CC4(CC4)NC2=O)C3(C)C)C(C)(C)C)CC1] |
| 169 | 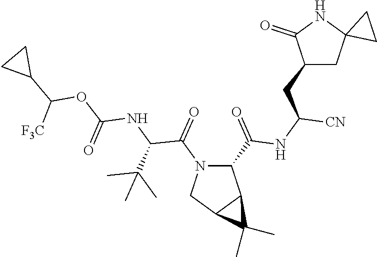 [SMILES: CC(C)(C)[C@H](NC(=O)OC(C1CC1)C(F)(F)F)C(=O)N1C[C@H]2[C@@H]([C@H]1C(=O)N[C@H](C#N)C[C@@H]1CC3(CC3)NC1=O)C2(C)C] |
| 170 | 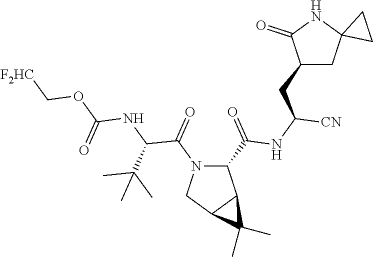 [SMILES: CC(C)(C)[C@H](NC(=O)OCC(F)F)C(=O)N1C[C@H]2[C@@H]([C@H]1C(=O)N[C@H](C#N)C[C@@H]1CC3(CC3)NC1=O)C2(C)C] |
| 171 | 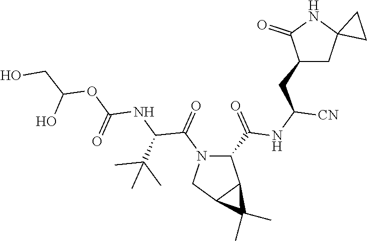 [SMILES: CC(C)(C)[C@H](NC(=O)OC(O)CO)C(=O)N1C[C@H]2[C@@H]([C@H]1C(=O)N[C@H](C#N)C[C@@H]1CC3(CC3)NC1=O)C2(C)C] |
| 172 | 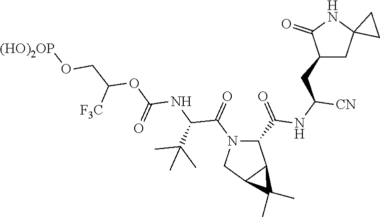 |
| 173 | 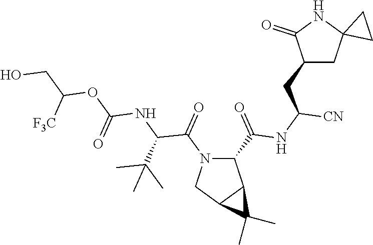 [SMILES: CC(C)(C)[C@H](NC(=O)OC(CO)C(F)(F)F)C(=O)N1C[C@H]2[C@@H]([C@H]1C(=O)N[C@H](C#N)C[C@@H]1CC3(CC3)NC1=O)C2(C)C] |
| 174 | 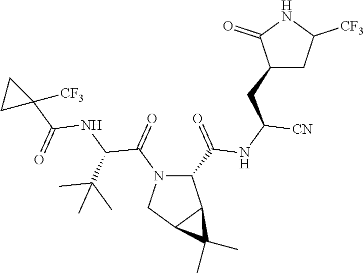 [SMILES: CC(C)(C)[C@H](NC(=O)C1(C(F)(F)F)CC1)C(=O)N1C[C@H]2[C@@H]([C@H]1C(=O)N[C@H](C#N)C[C@@H]1CC(C(F)(F)F)NC1=O)C2(C)C] |
| 175 | 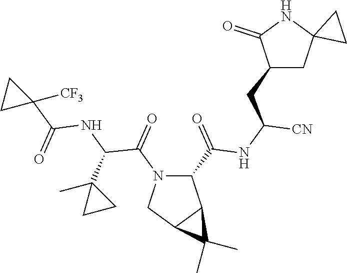 [SMILES: CC1([C@H](NC(=O)C2(C(F)(F)F)CC2)C(=O)N2C[C@H]3[C@@H]([C@H]2C(=O)N[C@H](C#N)C[C@@H]2CC4(CC4)NC2=O)C3(C)C)CC1] |
| 176 | 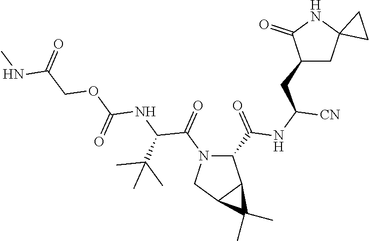 [SMILES: CNC(=O)COC(=O)N[C@H](C(=O)N1C[C@H]2[C@@H]([C@H]1C(=O)N[C@H](C#N)C[C@@H]1CC3(CC3)NC1=O)C2(C)C)C(C)(C)C] |
| 177 | 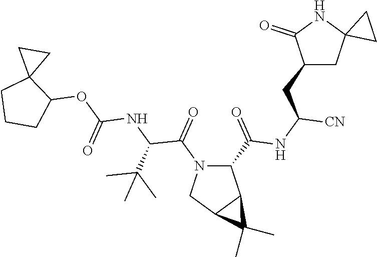 [SMILES: CC(C)(C)[C@H](NC(=O)OC1CCCC12CC2)C(=O)N1C[C@H]2[C@@H]([C@H]1C(=O)N[C@H](C#N)C[C@@H]1CC3(CC3)NC1=O)C2(C)C] |
| 178 | 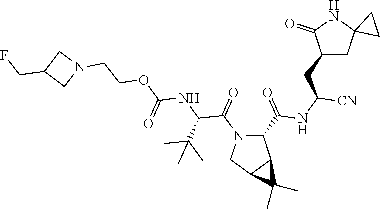 [SMILES: CC(C)(C)[C@H](NC(=O)OCCN1CC(CF)C1)C(=O)N1C[C@H]2[C@@H]([C@H]1C(=O)N[C@H](C#N)C[C@@H]1CC3(CC3)NC1=O)C2(C)C] |
| 179 | 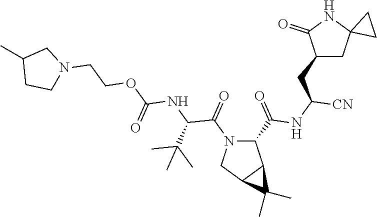 [SMILES: CC1CCN(CCOC(=O)N[C@H](C(=O)N2C[C@H]3[C@@H]([C@H]2C(=O)N[C@H](C#N)C[C@@H]2CC4(CC4)NC2=O)C3(C)C)C(C)(C)C)C1] |
| 180 | 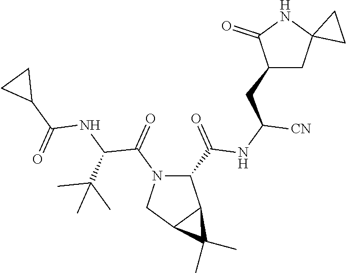 [SMILES: CC(C)(C)[C@H](NC(=O)C1CC1)C(=O)N1C[C@H]2[C@@H]([C@H]1C(=O)N[C@H](C#N)C[C@@H]1CC3(CC3)NC1=O)C2(C)C] |
| 181 | 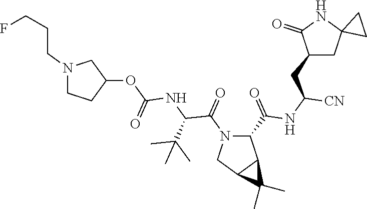 [SMILES: CC(C)(C)[C@H](NC(=O)OC1CCN(CCCF)C1)C(=O)N1C[C@H]2[C@@H]([C@H]1C(=O)N[C@H](C#N)C[C@@H]1CC3(CC3)NC1=O)C2(C)C] |
| 182 | 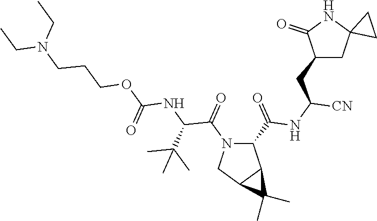 [SMILES: CCN(CC)CCCOC(=O)N[C@H](C(=O)N1C[C@H]2[C@@H]([C@H]1C(=O)N[C@H](C#N)C[C@@H]1CC3(CC3)NC1=O)C2(C)C)C(C)(C)C] |
| 183 | 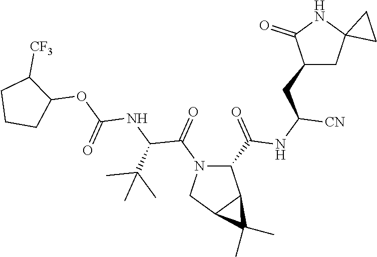 [SMILES: CC(C)(C)[C@H](NC(=O)OC1CCCC1C(F)(F)F)C(=O)N1C[C@H]2[C@@H]([C@H]1C(=O)N[C@H](C#N)C[C@@H]1CC3(CC3)NC1=O)C2(C)C] |
| 184 | 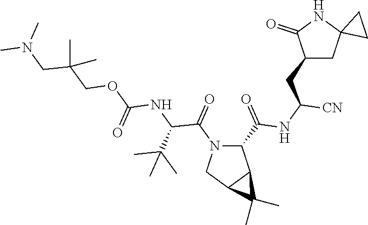 [SMILES: CN(C)CC(C)(C)COC(=O)N[C@H](C(=O)N1C[C@H]2[C@@H]([C@H]1C(=O)N[C@H](C#N)C[C@@H]1CC3(CC3)NC1=O)C2(C)C)C(C)(C)C] |
| 185 | 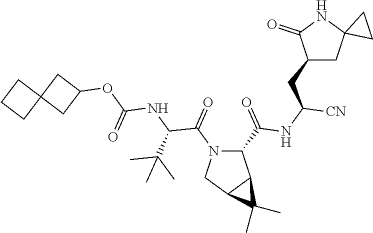 [SMILES: CC(C)(C)[C@H](NC(=O)OC1CC2(CCC2)C1)C(=O)N1C[C@H]2[C@@H]([C@H]1C(=O)N[C@H](C#N)C[C@@H]1CC3(CC3)NC1=O)C2(C)C] |
| 186 | 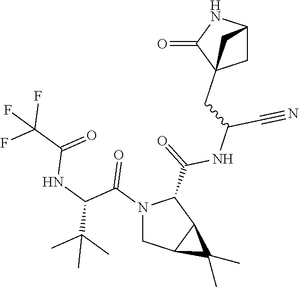 [SMILES: CC(C)(C)[C@H](NC(=O)C(F)(F)F)C(=O)N1C[C@H]2[C@@H]([C@H]1C(=O)NC(C#N)C[C@]13C[C@H](C1)NC3=O)C2(C)C] |

In embodiments, a disclosed compound is selected from the group consisting of

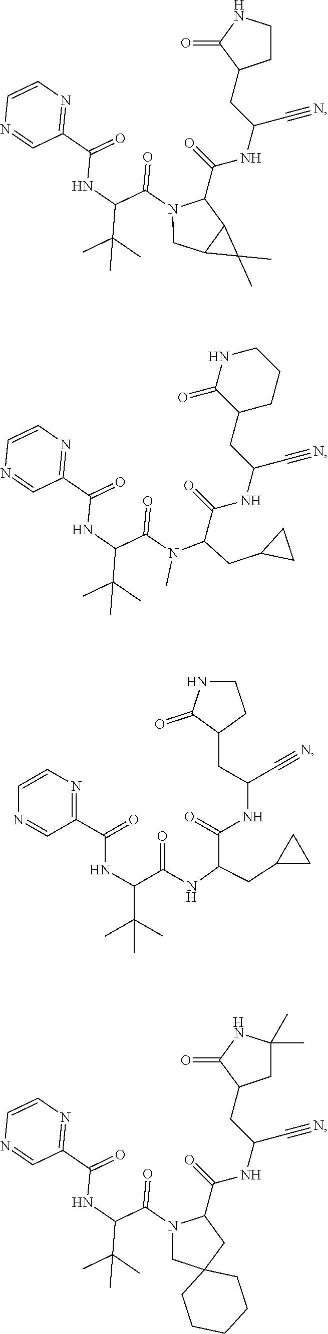

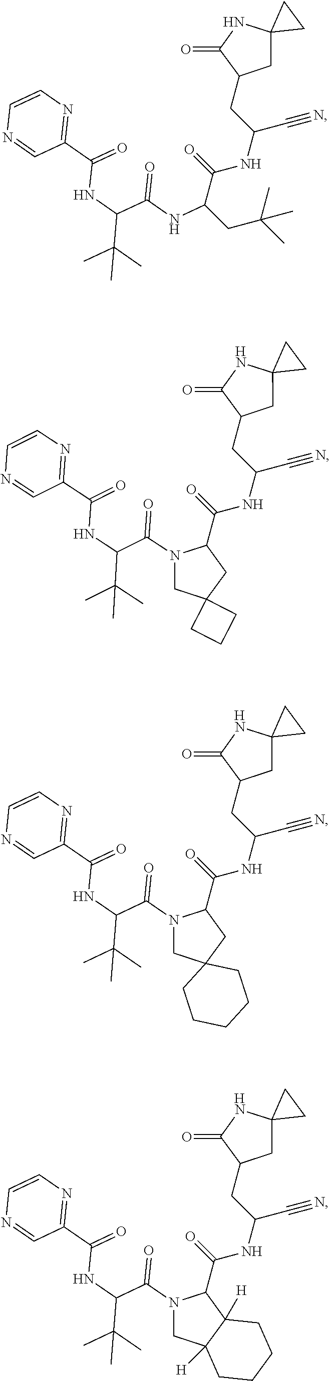

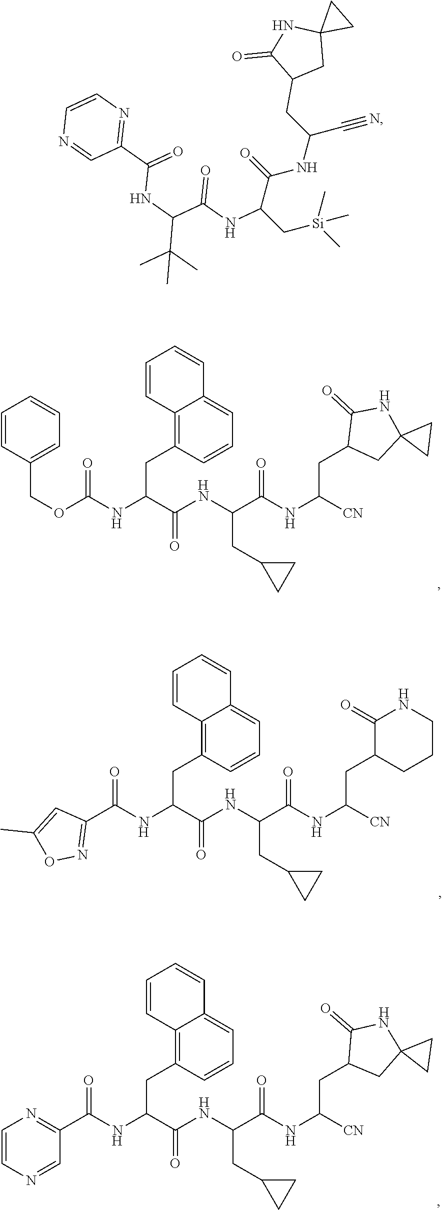

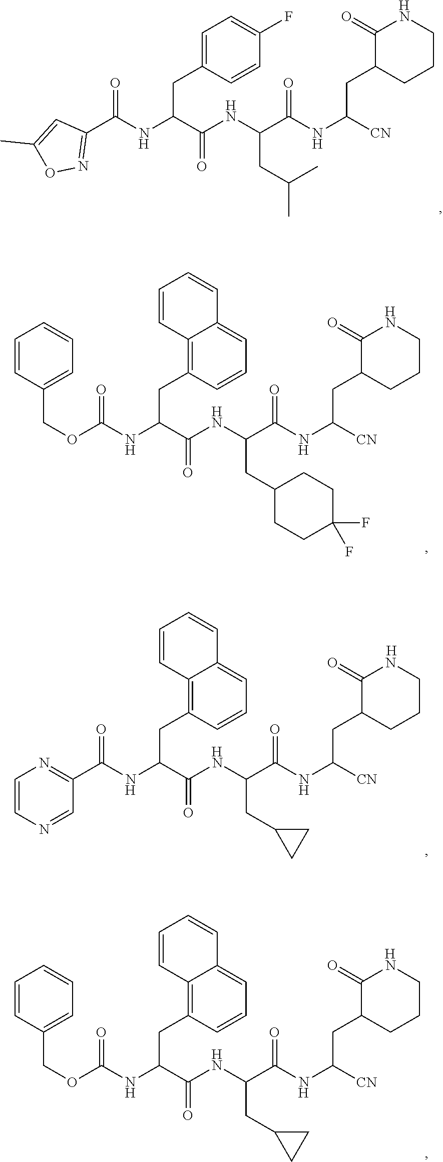

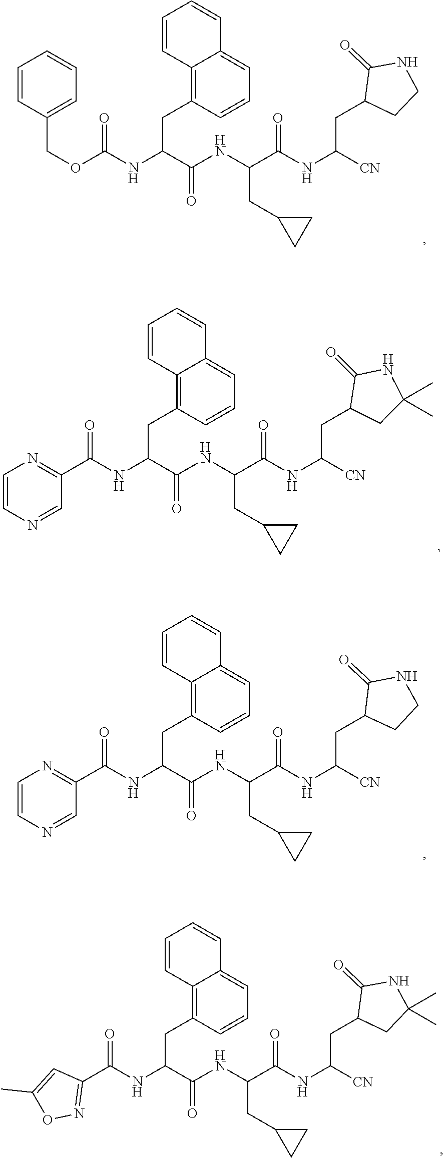

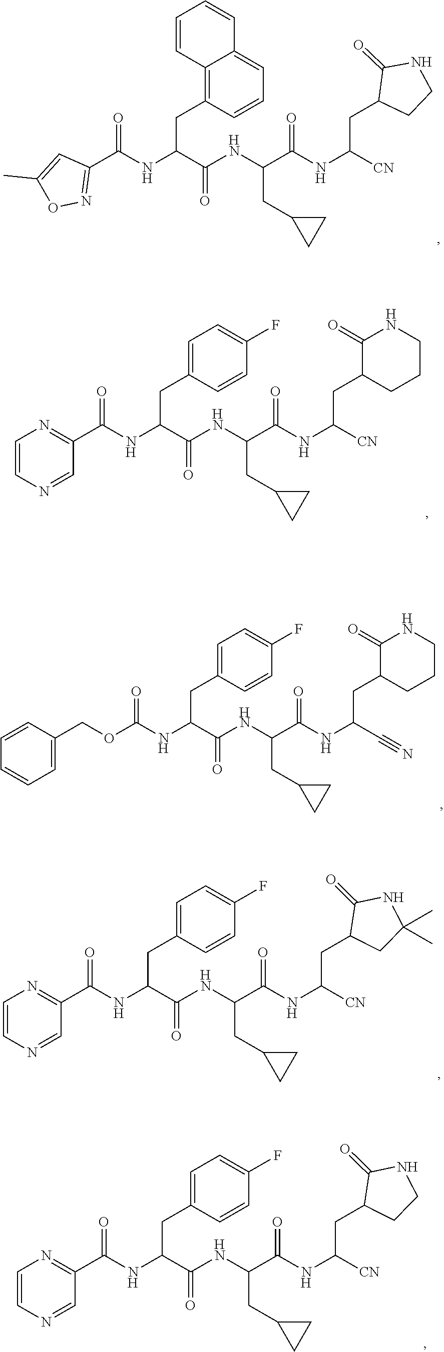

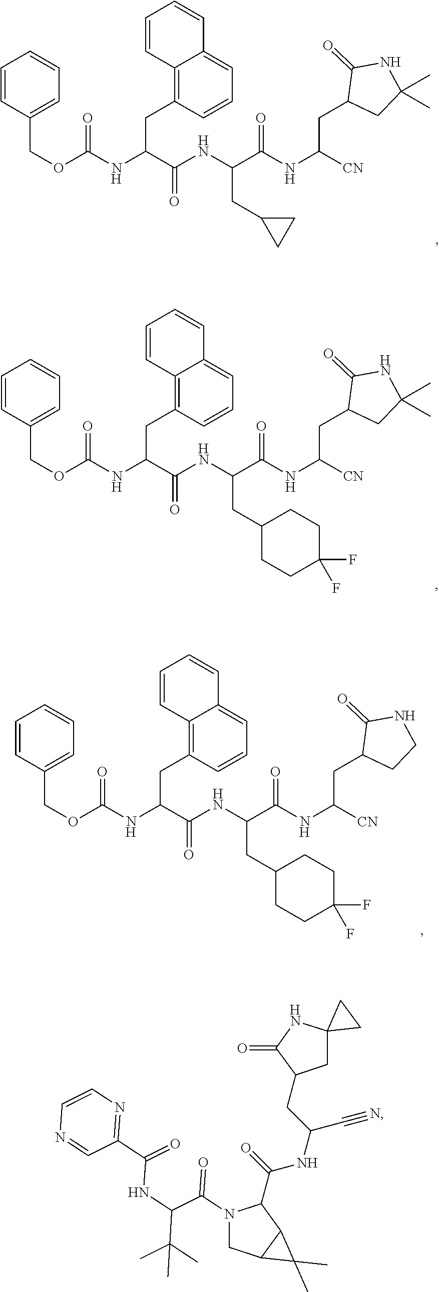

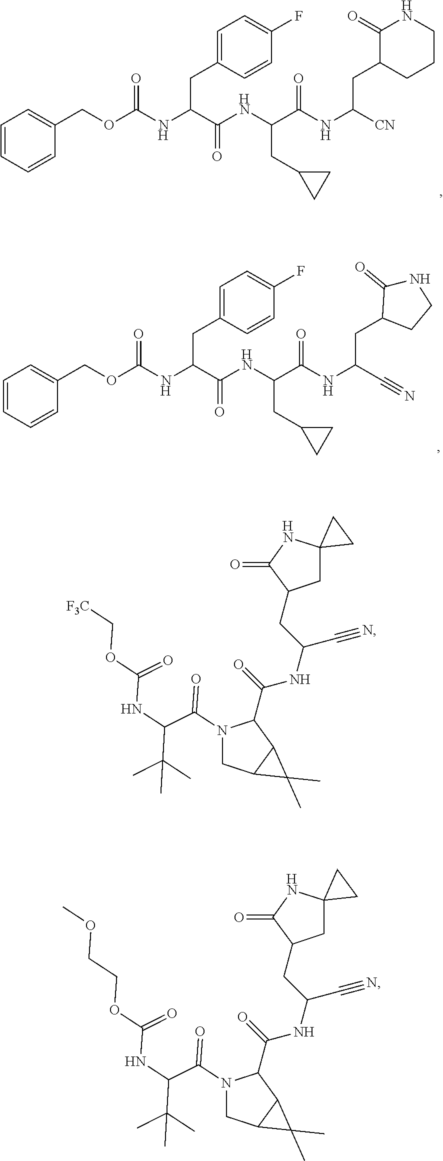

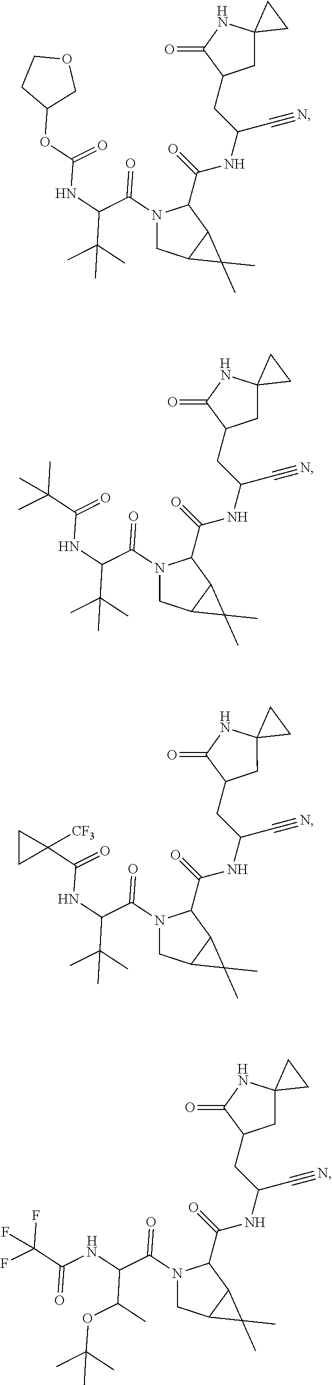

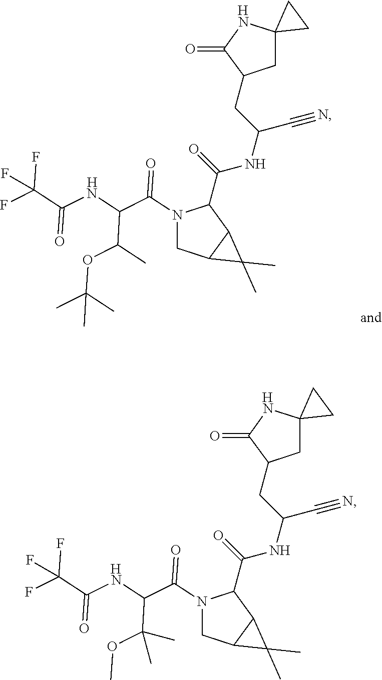

or pharmaceutically acceptable salt thereof.

Also disclosed herein is a protease inhibitory compound represented by Formula IIi:

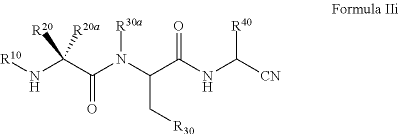

or a pharmaceutically acceptable salt or stereoisomer thereof, wherein: R¹⁰ is selected from the group consisting of hydrogen, —CH₂CF₃, —CH₂CF₂H, —CH(CF₃)₂, —CH(CF₃)CH₃, —C(CH₃)(CF₃)₂, —C(O)NR^{a}R¹⁰⁰, C₃-C₆cycloalkyl, phenyl, and 5-6 membered heteroaryl, wherein phenyl and 5-6 membered heteroaryl may optionally be substituted by or more substituents each independently selected from R⁵⁰; R^{a} is selected from hydrogen and C₁-C₃alkyl; R¹⁰⁰ is selected from the group consisting of hydrogen, C₁-C₆alkyl, C₃-C₁₀cycloalkyl, —C(R¹⁰¹)₂-C₆-C₁₀cycloalkyl, phenyl, —C(R¹⁰¹)₂-phenyl, 4-6 membered heterocyclyl, and 5-6 membered heteroaryl; wherein R¹⁰⁰ may optionally be substituted by one or more substituents each selected from R⁵⁰; or R^{a} and R¹⁰⁰, together with the nitrogen to which they are attached, may be joined together to form a 4-6 membered monocyclic or a 6-10 membered spirocyclic heterocyclyl optionally substituted by one or more halogens; R¹⁰¹ is selected from hydrogen and deuterium; R²⁰ is selected from the group consisting of C₁-C₆alkyl CH₂-R²²⁰; and C₃-C₆cycloalkyl, wherein R²⁰ or R²²⁰ may optionally be substituted by one, two, or three substituents each selected from R⁵⁰; R¹ is H; or R²⁰ and R¹, together with the carbon to which they are attached, may be joined together to form a C₃-C₆cycloalkyl or 4-6 membered monocyclic heterocyclyl; R²²⁰ is selected from the group consisting of phenyl, naphthyl, and 8-10 membered bicyclic heteroaryl; R is hydrogen or C₁-C₃alkyl; R³⁰ is selected from the group consisting of C₁-C₆alkyl, C₃-C₆cycloalkyl, phenyl, 4-6 heterocyclyl and 5-6 membered heteroaryl; wherein R³⁰ may optionally be substituted by one, two, or three substituents each selected from R⁵⁰; or R³⁰ is —Si(CH₃)₃; or R^{30a} and R³⁰, together with the atoms to which they are attached, may be joined together to form a 4-6 membered monocyclic, 6-10 membered fused bicyclic, or 6-10 membered spirocyclic heterocyclyl which may optionally be substituted by one, two or three substituents each selected from R⁵⁰; R⁴⁰ is selected from

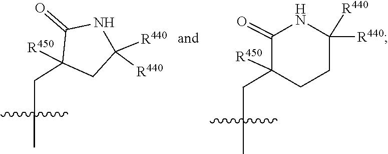

R⁴⁴⁰ is each independently selected from the group consisting of hydrogen, —CH₃ and —CF₃, or two R⁴⁴⁰ groups, together with the carbon to which they are attached, may be joined together to form a cyclopropyl, cyclobutyl, or oxetanyl; R⁴⁵⁰ is selected from hydrogen and C₁-C₃alkyl; or one R⁴⁴⁰ and R⁴⁵⁰, together with the atoms to which they are attached, may be joined together to form a 4-6 membered carbocycle; R⁵⁰ is independently selected for each occurrence from the group consisting of halogen, hydroxyl, cyano, —N(R⁵⁵⁰)₂, —N(R⁵⁵⁰)C(O)R⁵⁵⁰, —C(O)N(R⁵⁵⁰)₂, —OPO(OH)₂, —CO₂H, —SO₂CH₃ —CF₃, —CHF₂, C₁-C₆alkyl, C₁-C₆alkoxy, and phenyl; wherein C₁-C₆alkyl, C₁-C₆alkoxy and phenyl may optionally be substituted by one or more substituents each independently selected from the group consisting of halogen and hydroxyl; and R⁵⁵⁰ is selected from hydrogen and C₁-C₃alkyl, wherein the alkyl is optionally substituted by one, two or three halogens; or two R⁵⁵ groups, together with the atoms to which they are attached, may be joined together to form a 4-6 membered heterocyclyl, wherein the heterocycicyl may optionally be substituted by one, two or three substituents each independently selected from halogen, hydroxyl and C₁-C₃alkyl (optionally substituted by one or more halogens).

In some embodiments, R⁴⁰ is selected from

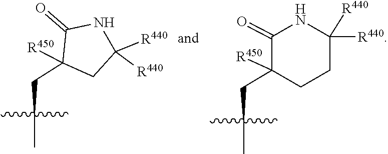

Also disclosed herein is a protease inhibitory compound represented by Formula II:

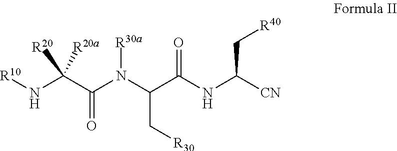

or a pharmaceutically acceptable salt or stereoisomer thereof, wherein: R¹⁰ is selected from the group consisting of hydrogen, —CH₂CF₃, —CH₂CF₂H, —CH(CF₃)₂, —CH(CF₃)CH₃, —C(CH₃)(CF₃)₂, —C(O)NR^{a}R¹⁰⁰, C₃-C₆cycloalkyl, phenyl, and 5-6 membered heteroaryl, wherein phenyl and 5-6 membered heteroaryl may optionally be substituted by or more substituents each independently selected from R⁵⁰; R is hydrogen or C₁-C₃alkyl; R¹⁰⁰ is selected from the group consisting of hydrogen, C₁-C₆alkyl, C₃-C₁₀cycloalkyl, —C(R¹⁰¹)₂-C₆-C₁₀cycloalkyl, phenyl, —C(R¹⁰¹)₂-phenyl, 4-6 membered heterocyclyl, and 5-6 membered heteroaryl; wherein R¹⁰⁰ may optionally be substituted by one or more substituents each selected from R⁵⁰; or R^{a} and R¹⁰⁰, together with the nitrogen to which they are attached, may be joined together to form a 4-6 membered monocyclic or a 6-10 membered spirocyclic heterocyclyl optionally substituted by one or more halogens; R¹⁰¹ is hydrogen or deuterium; R²⁰ is selected from the group consisting of C₁-C₆alkyl CH₂-R²²⁰; and C₃-C₆cycloalkyl, wherein R²⁰ or R²²⁰ may optionally be substituted by one, two, or three substituents each selected from R⁵⁰; R is H; or R²⁰ and R², together with the carbon to which they are attached, may be joined together to form a C₃-C₆cycloalkyl or 4-6 membered monocyclic heterocyclyl; R²²⁰ is selected from the group consisting of phenyl, naphthyl, and 8-10 membered bicyclic heteroaryl; R is hydrogen or C₁-C₃alkyl; R³⁰ is selected from the group consisting of C₁-C₆alkyl, C₃-C₆cycloalkyl, phenyl, 4-6 heterocyclyl and 5-6 membered heteroaryl; wherein R³⁰ may optionally be substituted by one, two, or three substituents each selected from R⁵⁰; or R³⁰ is —Si(CH₃)₃; or R^{30a} and R³⁰, together with the atoms to which they are attached, may be joined together to form a 4-6 membered monocyclic, 6-10 membered fused bicyclic, or 6-10 membered spirocyclic heterocyclyl which may optionally be substituted by one, two or three substituents each selected from R⁵⁰; R⁴⁰ is

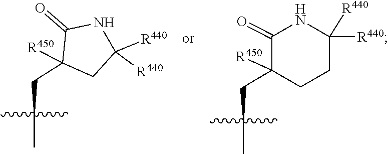

R⁴⁴⁰ is each independently selected from the group consisting of hydrogen, —CH₃ and —CF₃, or two R groups, together with the carbon to which they are attached, may be joined together to form a cyclopropyl, cyclobutyl, or oxetanyl; R⁴⁵⁰ is hydrogen; or one R⁴° and R⁴⁵⁰, together with the atoms to which they are attached, may be joined together to form a 4-6 membered carbocycle; R⁵⁰ is independently selected for each occurrence from the group consisting of halogen, hydroxyl, cyano, —N(R⁵⁵⁰)₂, —N(R⁵⁵⁰)C(O)R⁵⁵⁰, —C(O)N(R⁵⁵⁰)₂, —OPO(OH)₂, —CO₂H, —SO₂CH₃ —CF₃, —CHF₂, C₁-C₆alkyl, C₁-C₆alkoxy, and phenyl; wherein C₁-C₆alkyl, C₁-C₆alkoxy and phenyl may optionally be substituted by one or more substituents each independently selected from the group consisting of halogen and hydroxyl; and R⁵⁵⁰ is hydrogen or C₁-C₃alkyl optionally substituted by one, two or three halogens; or two R⁵⁵ groups, together with the atoms to which they are attached, may be joined together to form a 4-6 membered heterocyclyl, wherein the heterocycicyl may optionally be substituted by one, two or three substituents each independently selected from halogen, hydroxyl and C₁-C₃alkyl(optionally substituted by one or more halogens).

In some embodiments, disclosed herein is a protease inhibitory compound represented by Formula II:

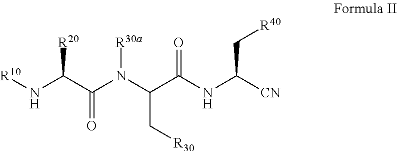

or a pharmaceutically acceptable salt or stereoisomer thereof, wherein: R¹⁰ is selected from the group consisting of —CH₂CF₃, —CH²CF₂H, —CH(CF₃)₂, —CH(CF₃)CH₃ and —C(O)NHR¹⁰⁰; R¹⁰⁰ is selected from the group consisting of hydrogen, C₁-C₆alkyl, C₃-C₁₀cycloalkyl, —C(R¹⁰¹)₂-C₆-C₁₀cycloalkyl, phenyl, —C(R¹⁰ z-phenyl and 5-6 membered heteroaryl; wherein R¹⁰ may optionally be substituted by one, two, or three substituents each selected from R⁵⁰; R¹⁰¹ is hydrogen or deuterium; R²⁰ is C₁-C₆alkyl or CH₂-R²²⁰; wherein R²⁰ or R²²⁰ may optionally be substituted by one, two, or three substituents each selected from R⁵⁰; R²²⁰ is selected from the group consisting of phenyl, naphthyl, and 8-10 membered bicyclic heteroaryl; R is hydrogen or C₁-C₃alkyl; R³⁰ is selected from the group consisting of C₁-C₆alkyl, C₃-C₆cycloalkyl, phenyl and 4-6 heterocyclyl and 5-6 membered heteroaryl; wherein R³⁰ may optionally be substituted by one, two, or three substituents each selected from R⁵⁰; or R³⁰ is —Si(CH₃)₃; or R and R³⁰, together with the atoms to which they are attached, may be joined together to form a 4-6 membered monocyclic, 6-10 membered fused bicyclic, or 6-10 membered spirocyclic heterocycle which may optionally be substituted by one, two or three substituents each selected from R⁵⁰; R⁴⁰ is

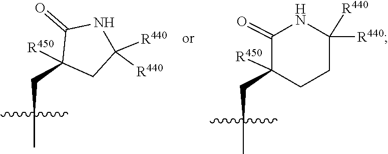

R⁴⁴⁰ is each independently hydrogen or —CH₃, or two R⁴⁴⁰ groups, together with the carbon to which they are attached, may be joined together to form a cyclopropyl, cyclobutyl, or oxetanyl; R⁵⁰ is independently selected for each occurrence from the group consisting of halogen, hydroxyl, cyano, —N(R⁵⁵⁰)₂, —N(R⁵⁵⁰)C(O)R⁵⁵⁰, —C(O)N(R⁵⁵⁰)₂, —CF₃, C₁-C₆alkyl, C₁-C₆alkoxy, and phenyl; wherein C₁-C₆alkyl, C₁-C₆alkoxy and phenyl may optionally be substituted by one or more substituents each independently selected from the group consisting of halogen and hydroxyl; and R⁵⁵⁰ is hydrogen or C₁-C₃alkyl optionally substituted by one, two or three halogens.

In some embodiments, the compound is represented by:

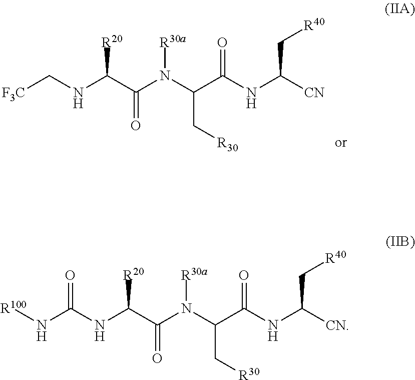

In some embodiments, the compound is represented by:

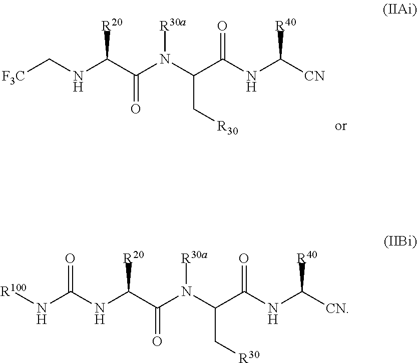

In some embodiments, R¹⁰ is selected from the group consisting of:

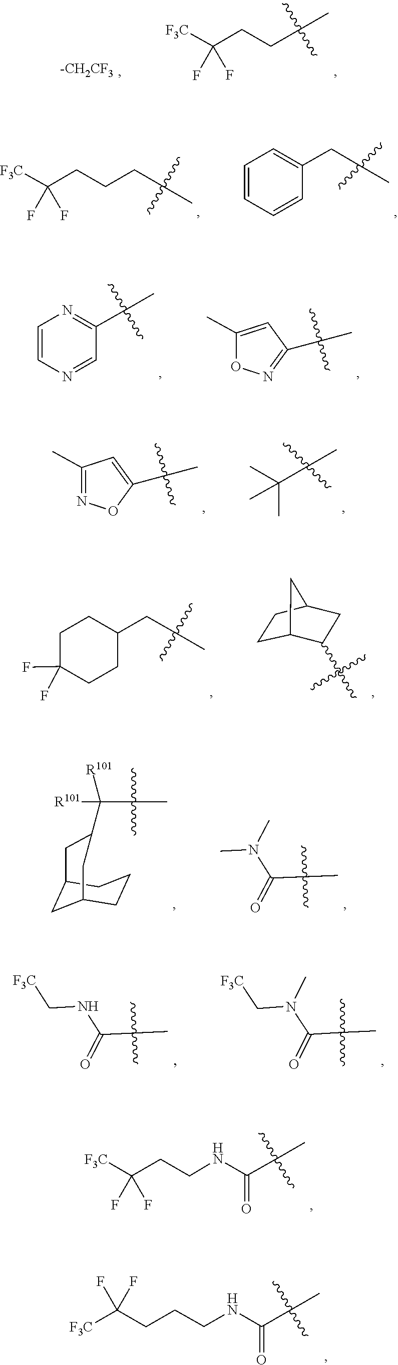

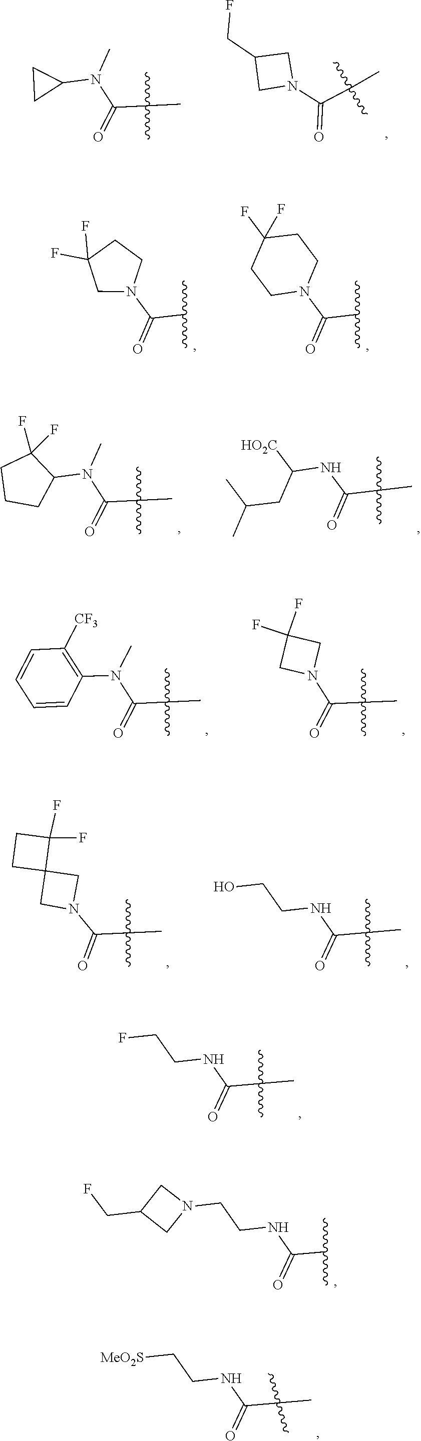

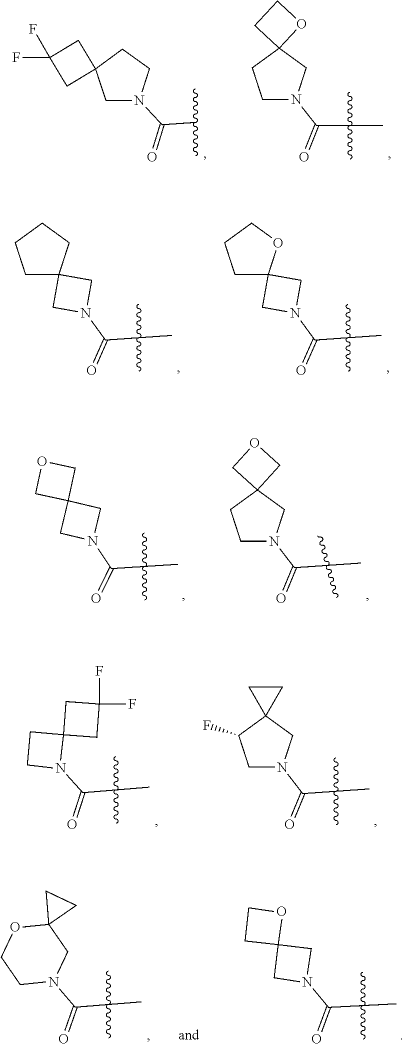

In some embodiments, R¹⁰ is 5-6 membered heteroaryl.

In some embodiments, R¹⁰ is selected from the group consisting of:

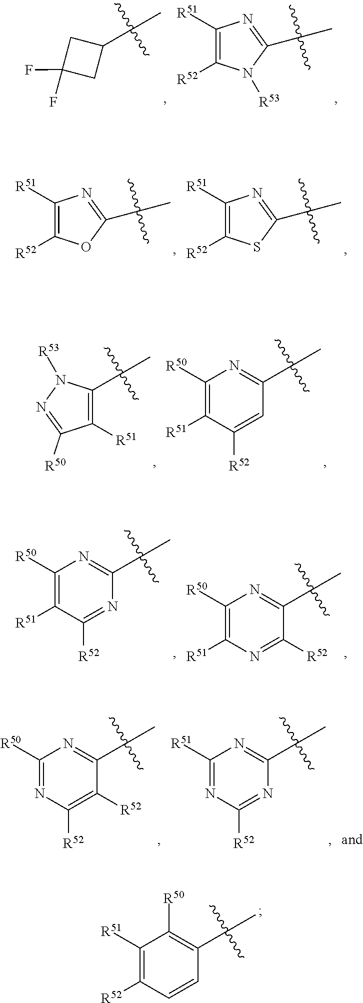

wherein R⁵⁰, R⁵¹ and R⁵² are each independently selected from the group consisting of chloro, fluoro, C₁-C₃alkyl and C₁-C₃alkoxy, wherein C₁-C₃alkyl and C₁-C₃alkoxy may optionally be substituted by one, two or three halogens; and wherein R⁵³ is hydrogen or C₁-C₃alkyl optionally substituted by one, two or three halogens.

In other embodiments, R²⁰ is selected from the group consisting of:

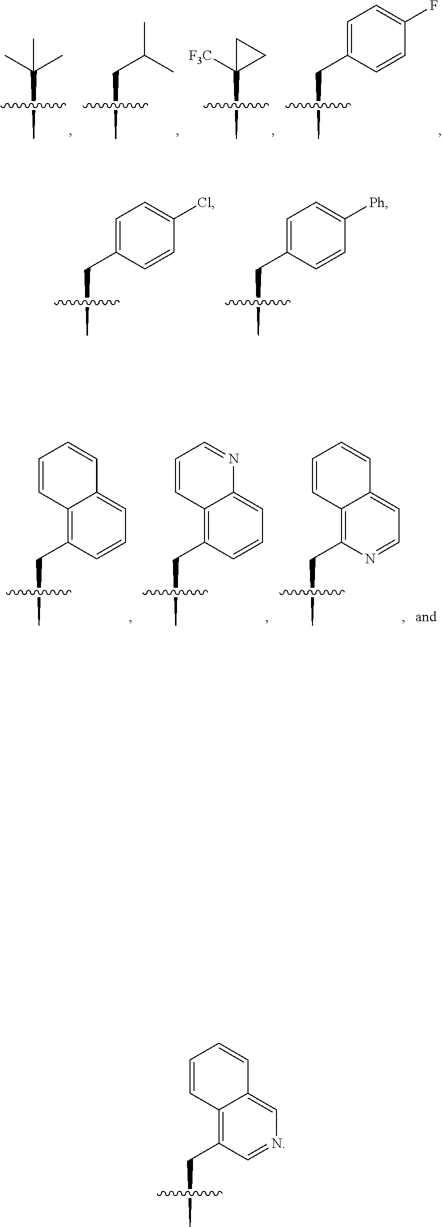

In some embodiments, R²⁰ is selected from the group consisting of

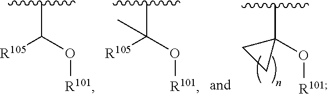

wherein R¹⁰⁵ is C₁-C₆alkyl, and n is 1, 2, or 3; R¹⁰¹ is selected from the group consisting of C₁-C₆alkyl, C₂-C₆alkenyl, C₃-C₆cycloalkyl, and C₃-C₆cycloalkenyl, wherein R¹⁰¹ may optionally be substituted by one, two, three, or four substituents each independently selected from R^{D}; R^{D} is selected from the group consisting of halogen, C₁-C₆alkyl, C₁-C₆alkoxy, C₃-C₆cycloalkyl, and cyano, wherein the alkyl may optionally be substituted by one or more halo.

In some embodiments, R²⁰ is selected from the group consisting of

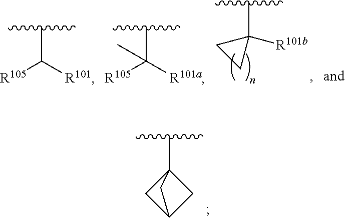

wherein R¹⁰⁵ is C₁-C₂alkyl, bb is 1 or 2, R^{101a} and R^{101b} is each independently selected from the group consisting of C₁-C₆alkyl, C₂-C₆alkenyl, C₃-C₆cycloalkyl, and C₃-C₆cycloalkenyl, wherein R^{101a} or R^{101b} may optionally be substituted by one, two, three, or four substituents each independently selected from R^{D}; R^{D} is selected from the group consisting of halogen, C₁-C₆alkyl, C₁-C₆alkoxy, C₃-C₆cycloalkyl, and cyano, wherein the alkyl may optionally be substituted by one or more halo.

In some embodiments, R²⁰ is selected from the group consisting of

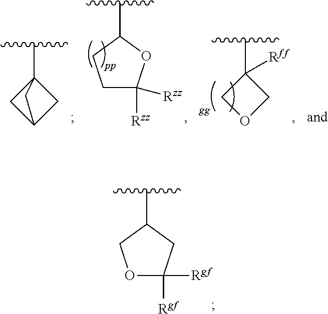

wherein pp is 0, 1, 2 or 3, R^{zz} is each independently H or C₁-C₆alkyl(e.g., CH₃), gg is 1, 2 or 3, R^{ff} is H or C₁-C₆alkyl(e.g., CH₃), and R^{gf} is each independently H or C₁-C₆alkyl(e.g., CH₃). In some embodiments, pp is 0 or 1. In some embodiments, gg is 1. In some embodiments, R^{ff} is methyl. In some embodiments, R^{ff} is H. In some embodiments, R^{gf} is H. In some embodiments, R^{gf} is methyl.

In further embodiments, R is hydrogen. In certain embodiments, R³⁰ is selected from the group consisting of:

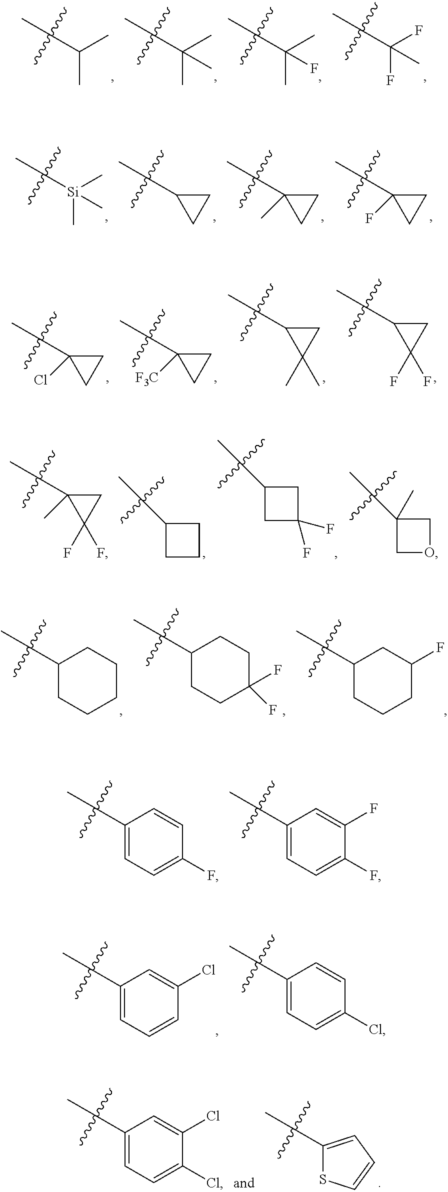

In some embodiments, R and R³⁰, together with the atoms to which they are attached, join together to form a 4-6 membered monocyclic, 6-10 membered fused bicyclic, or 6-10 membered spirocyclic heterocycle.

In other embodiments, the compound is represented by:

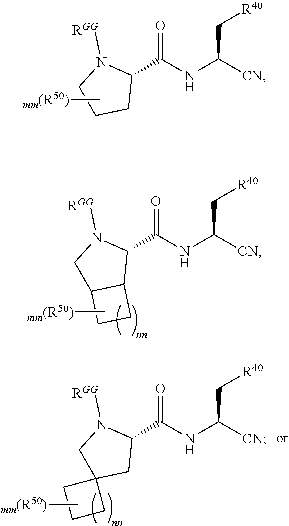

wherein mm is selected from 0, 1, 2 and 3; nn is selected from 0, 1, 2 and 3; and R^{GG} is

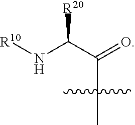

In other embodiments, the compound is represented by:

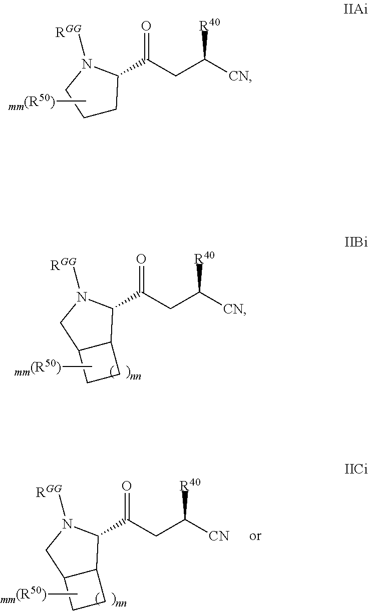

wherein mm is selected from 0, 1, 2 and 3; nn is selected from 0, 1, 2 and 3; and R^{GG} is

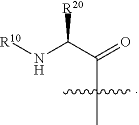

In still other embodiments, R³ and R³⁰, together with the atoms to which they are attached, join together to form a 4-6 membered monocyclic, 6-10 membered fused bicyclic, or 6-10 membered spirocyclic heterocycle selected from the group consisting of:

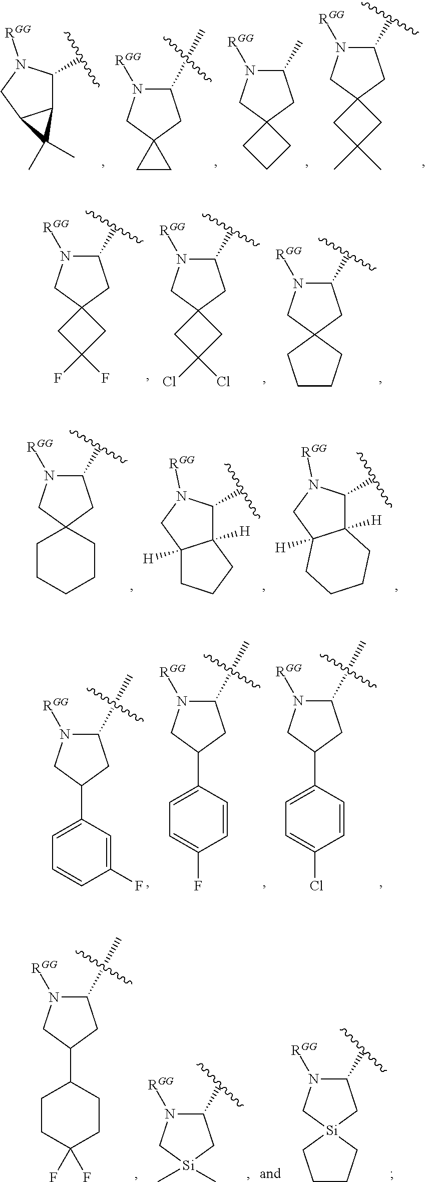

wherein R^{GG} is

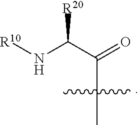

In certain embodiments, R⁴⁰ is selected from the group consisting of:

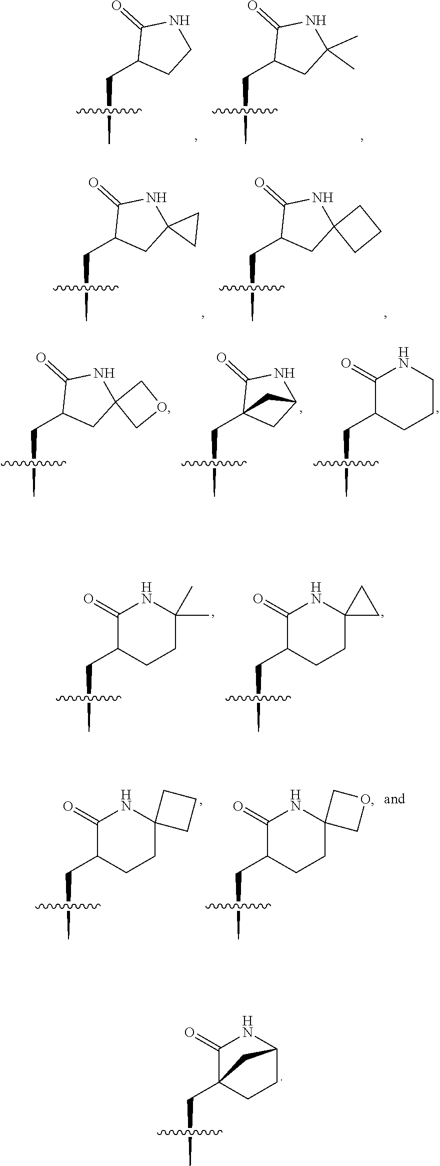

In further embodiments, R⁵ is selected from the group consisting of chloro, fluoro, —CF₃, —CH₃ and phenyl.

In some embodiments, a disclosed compound is selected from the group consisting of the compounds identified in Table 2, or pharmaceutically acceptable salt thereof.

TABLE 2

| Cmpd | |
|---|---|
| No. | Structure |
| 201 | 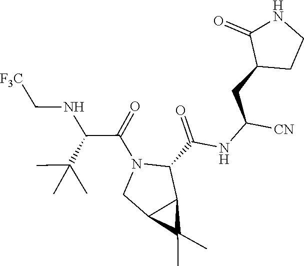 [SMILES: CC(C)(C)[C@H](NCC(F)(F)F)C(=O)N1C[C@H]2[C@@H]([C@H]1C(=O)N[C@H](C#N)C[C@@H]1CCNC1=O)C2(C)C] |
| 202 | 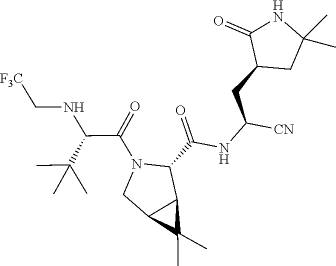 [SMILES: CC1(C)C[C@@H](C[C@@H](C#N)NC(=O)[C@@H]2[C@@H]3[C@H](CN2C(=O)[C@@H](NCC(F)(F)F)C(C)(C)C)C3(C)C)C(=O)N1] |
| 203 | 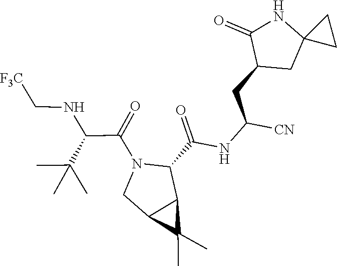 [SMILES: CC(C)(C)[C@H](NCC(F)(F)F)C(=O)N1C[C@H]2[C@@H]([C@H]1C(=O)N[C@H](C#N)C[C@@H]1CC3(CC3)NC1=O)C2(C)C] |
| 204 | 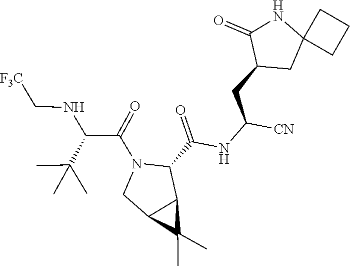 [SMILES: CC(C)(C)[C@H](NCC(F)(F)F)C(=O)N1C[C@H]2[C@@H]([C@H]1C(=O)N[C@H](C#N)C[C@@H]1CC3(CCC3)NC1=O)C2(C)C] |
| 205 | 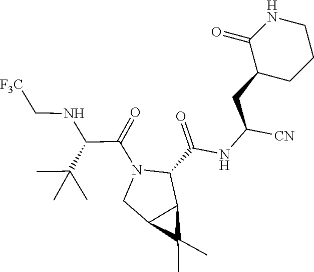 [SMILES: CC(C)(C)[C@H](NCC(F)(F)F)C(=O)N1C[C@H]2[C@@H]([C@H]1C(=O)N[C@H](C#N)C[C@@H]1CCCNC1=O)C2(C)C] |
| 206 | 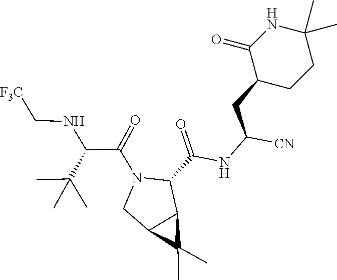 [SMILES: CC1(C)CC[C@@H](C[C@@H](C#N)NC(=O)[C@@H]2[C@@H]3[C@H](CN2C(=O)[C@@H](NCC(F)(F)F)C(C)(C)C)C3(C)C)C(=O)N1] |
| 207 | 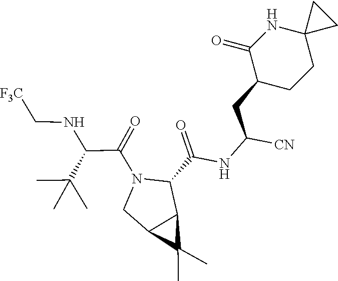 [SMILES: CC(C)(C)[C@H](NCC(F)(F)F)C(=O)N1C[C@H]2[C@@H]([C@H]1C(=O)N[C@H](C#N)C[C@@H]1CCC3(CC3)NC1=O)C2(C)C] |
| 208 | 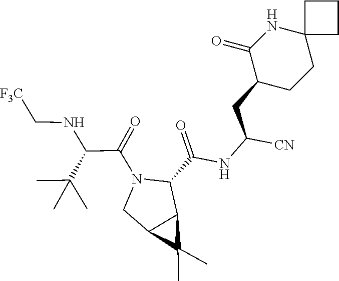 [SMILES: CC(C)(C)[C@H](NCC(F)(F)F)C(=O)N1C[C@H]2[C@@H]([C@H]1C(=O)N[C@H](C#N)C[C@@H]1CCC3(CCC3)NC1=O)C2(C)C] |
| 209 | 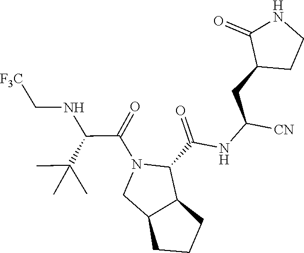 [SMILES: CC(C)(C)[C@H](NCC(F)(F)F)C(=O)N1C[C@@H]2CCC[C@@H]2[C@H]1C(=O)N[C@H](C#N)C[C@@H]1CCNC1=O] |
| 210 | 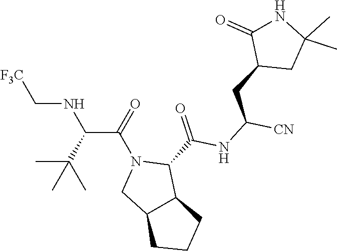 [SMILES: CC1(C)C[C@@H](C[C@@H](C#N)NC(=O)[C@@H]2[C@H]3CCC[C@H]3CN2C(=O)[C@@H](NCC(F)(F)F)C(C)(C)C)C(=O)N1] |
| 211 | 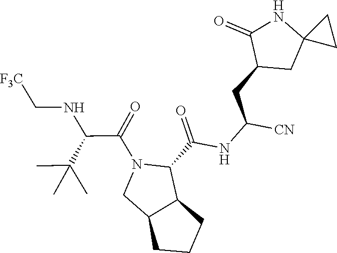 [SMILES: CC(C)(C)[C@H](NCC(F)(F)F)C(=O)N1C[C@@H]2CCC[C@@H]2[C@H]1C(=O)N[C@H](C#N)C[C@@H]1CC2(CC2)NC1=O] |
| 212 | 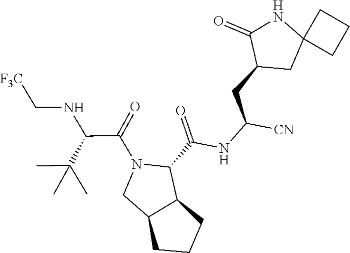 [SMILES: CC(C)(C)[C@H](NCC(F)(F)F)C(=O)N1C[C@@H]2CCC[C@@H]2[C@H]1C(=O)N[C@H](C#N)C[C@@H]1CC2(CCC2)NC1=O] |
| 213 | 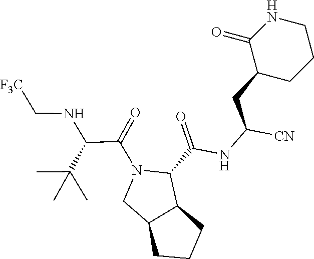 [SMILES: CC(C)(C)[C@H](NCC(F)(F)F)C(=O)N1C[C@@H]2CCC[C@@H]2[C@H]1C(=O)N[C@H](C#N)C[C@@H]1CCCNC1=O] |
| 214 | 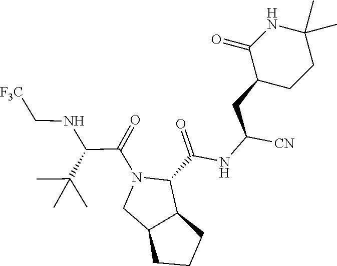 [SMILES: CC1(C)CC[C@@H](C[C@@H](C#N)NC(=O)[C@@H]2[C@H]3CCC[C@H]3CN2C(=O)[C@@H](NCC(F)(F)F)C(C)(C)C)C(=O)N1] |
| 215 | 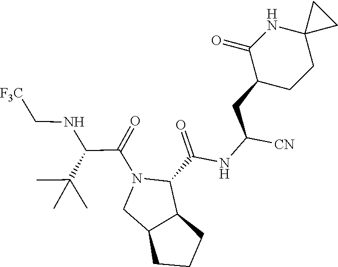 [SMILES: CC(C)(C)[C@H](NCC(F)(F)F)C(=O)N1C[C@@H]2CCC[C@@H]2[C@H]1C(=O)N[C@H](C#N)C[C@@H]1CCC2(CC2)NC1=O] |
| 216 | 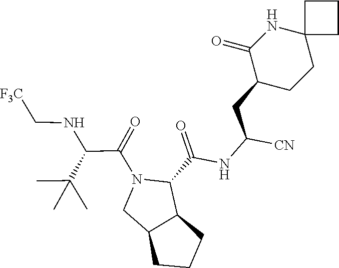 [SMILES: CC(C)(C)[C@H](NCC(F)(F)F)C(=O)N1C[C@@H]2CCC[C@@H]2[C@H]1C(=O)N[C@H](C#N)C[C@@H]1CCC2(CCC2)NC1=O] |
| 217 | 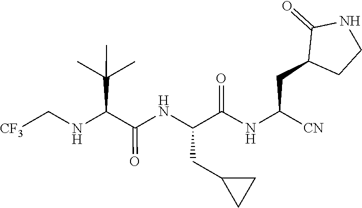 [SMILES: CC(C)(C)[C@H](NCC(F)(F)F)C(=O)N[C@@H](CC1CC1)C(=O)N[C@H](C#N)C[C@@H]1CCNC1=O] |
| 218 | 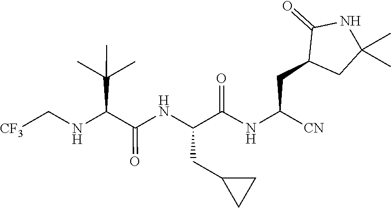 [SMILES: CC1(C)C[C@@H](C[C@@H](C#N)NC(=O)[C@H](CC2CC2)NC(=O)[C@@H](NCC(F)(F)F)C(C)(C)C)C(=O)N1] |
| 219 | 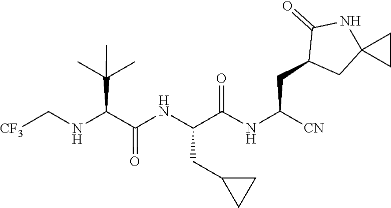 [SMILES: CC(C)(C)[C@H](NCC(F)(F)F)C(=O)N[C@@H](CC1CC1)C(=O)N[C@H](C#N)C[C@@H]1CC2(CC2)NC1=O] |
| 220 | 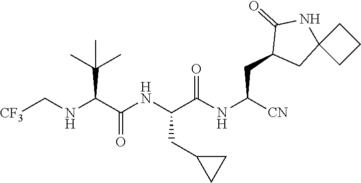 [SMILES: CC(C)(C)[C@H](NCC(F)(F)F)C(=O)N[C@@H](CC1CC1)C(=O)N[C@H](C#N)C[C@@H]1CC2(CCC2)NC1=O] |
| 221 | 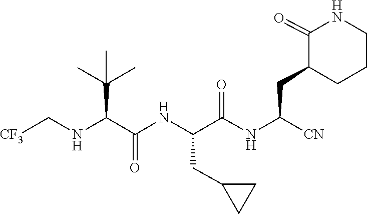 [SMILES: CC(C)(C)[C@H](NCC(F)(F)F)C(=O)N[C@@H](CC1CC1)C(=O)N[C@H](C#N)C[C@@H]1CCCNC1=O] |
| 222 | 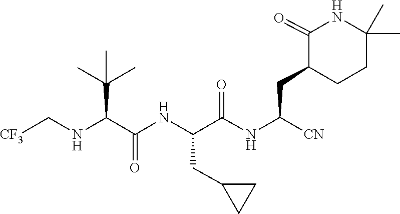 [SMILES: CC1(C)CC[C@@H](C[C@@H](C#N)NC(=O)[C@H](CC2CC2)NC(=O)[C@@H](NCC(F)(F)F)C(C)(C)C)C(=O)N1] |
| 223 | 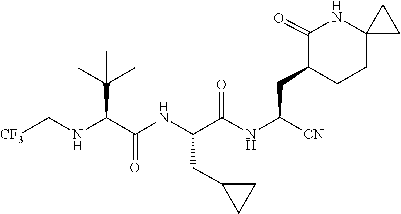 [SMILES: CC(C)(C)[C@H](NCC(F)(F)F)C(=O)N[C@@H](CC1CC1)C(=O)N[C@H](C#N)C[C@@H]1CCC2(CC2)NC1=O] |
| 224 | 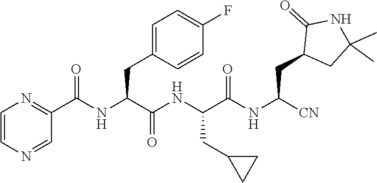 [SMILES: CC1(C)C[C@@H](C[C@@H](C#N)NC(=O)[C@H](CC2CC2)NC(=O)[C@H](Cc2ccc(F)cc2)NC(=O)c2cnccn2)C(=O)N1] |
| 225 | 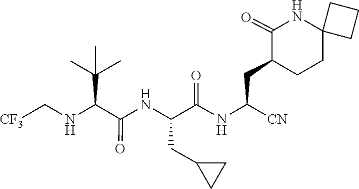 [SMILES: CC(C)(C)[C@H](NCC(F)(F)F)C(=O)N[C@@H](CC1CC1)C(=O)N[C@H](C#N)C[C@@H]1CCC2(CCC2)NC1=O] |
| 226 | 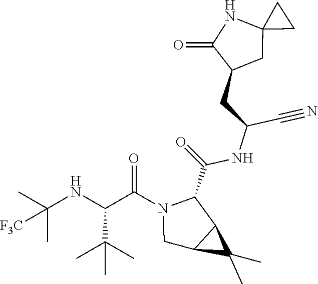 [SMILES: CC(C)(C)[C@H](NC(C)(C)C(F)(F)F)C(=O)N1C[C@H]2[C@@H]([C@H]1C(=O)N[C@H](C#N)C[C@@H]1CC3(CC3)NC1=O)C2(C)C] |
| 227 | 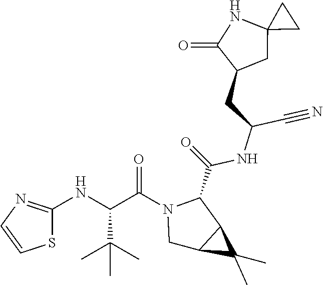 [SMILES: CC(C)(C)[C@H](Nc1nccs1)C(=O)N1C[C@H]2[C@@H]([C@H]1C(=O)N[C@H](C#N)C[C@@H]1CC3(CC3)NC1=O)C2(C)C] |
| 228 | 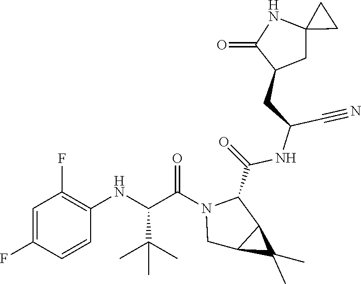 [SMILES: CC(C)(C)[C@H](Nc1ccc(F)cc1F)C(=O)N1C[C@H]2[C@@H]([C@H]1C(=O)N[C@H](C#N)C[C@@H]1CC3(CC3)NC1=O)C2(C)C] |
| 229 | 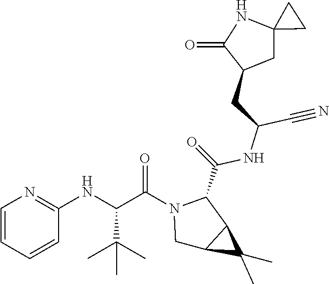 [SMILES: CC(C)(C)[C@H](Nc1ccccn1)C(=O)N1C[C@H]2[C@@H]([C@H]1C(=O)N[C@H](C#N)C[C@@H]1CC3(CC3)NC1=O)C2(C)C] |
| 230 | 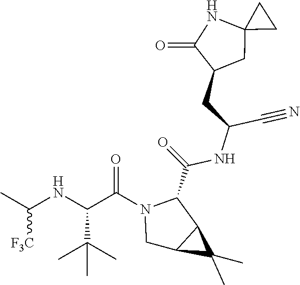 [SMILES: CC(N[C@H](C(=O)N1C[C@H]2[C@@H]([C@H]1C(=O)N[C@H](C#N)C[C@@H]1CC3(CC3)NC1=O)C2(C)C)C(C)(C)C)C(F)(F)F] |
| 231 | 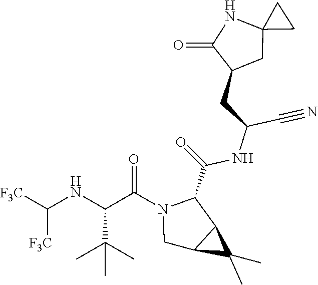 [SMILES: CC(C)(C)[C@H](NC(C(F)(F)F)C(F)(F)F)C(=O)N1C[C@H]2[C@@H]([C@H]1C(=O)N[C@H](C#N)C[C@@H]1CC3(CC3)NC1=O)C2(C)C] |
| 232 | 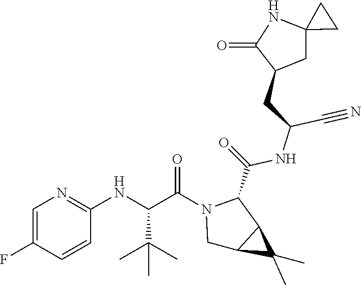 [SMILES: CC(C)(C)[C@H](Nc1ccc(F)cn1)C(=O)N1C[C@H]2[C@@H]([C@H]1C(=O)N[C@H](C#N)C[C@@H]1CC3(CC3)NC1=O)C2(C)C] |
| 233 | 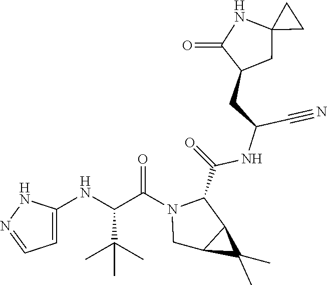 [SMILES: CC(C)(C)[C@H](Nc1ccn[nH]1)C(=O)N1C[C@H]2[C@@H]([C@H]1C(=O)N[C@H](C#N)C[C@@H]1CC3(CC3)NC1=O)C2(C)C] |
| 234 | 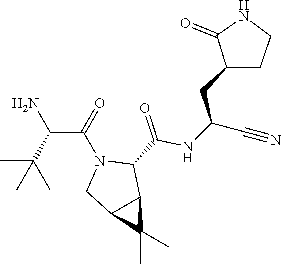 [SMILES: CC(C)(C)[C@H](N)C(=O)N1C[C@H]2[C@@H]([C@H]1C(=O)N[C@H](C#N)C[C@@H]1CCNC1=O)C2(C)C] |
| 235 | 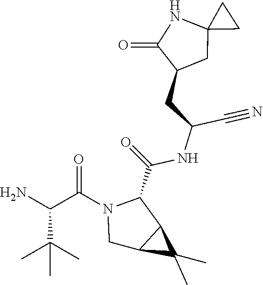 [SMILES: CC(C)(C)[C@H](N)C(=O)N1C[C@H]2[C@@H]([C@H]1C(=O)N[C@H](C#N)C[C@@H]1CC3(CC3)NC1=O)C2(C)C] |
| 236 | 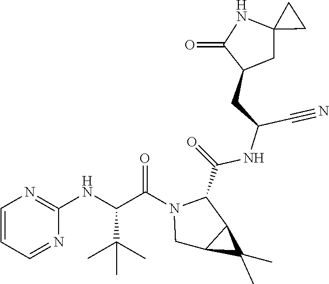 [SMILES: CC(C)(C)[C@H](Nc1ncccn1)C(=O)N1C[C@H]2[C@@H]([C@H]1C(=O)N[C@H](C#N)C[C@@H]1CC3(CC3)NC1=O)C2(C)C] |
| 237 | 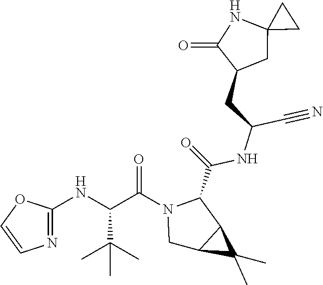 [SMILES: CC(C)(C)[C@H](Nc1ncco1)C(=O)N1C[C@H]2[C@@H]([C@H]1C(=O)N[C@H](C#N)C[C@@H]1CC3(CC3)NC1=O)C2(C)C] |
| 238 | 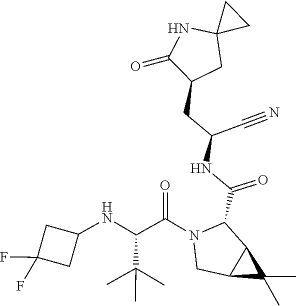 [SMILES: CC(C)(C)[C@H](NC1CC(F)(F)C1)C(=O)N1C[C@H]2[C@@H]([C@H]1C(=O)N[C@H](C#N)C[C@@H]1CC3(CC3)NC1=O)C2(C)C] |
| 239 | 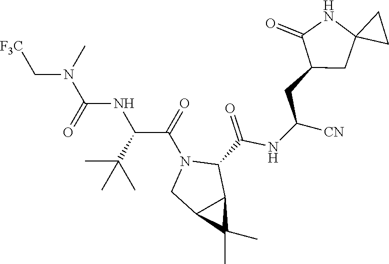 [SMILES: CN(CC(F)(F)F)C(=O)N[C@H](C(=O)N1C[C@H]2[C@@H]([C@H]1C(=O)N[C@H](C#N)C[C@@H]1CC3(CC3)NC1=O)C2(C)C)C(C)(C)C] |
| 240 | 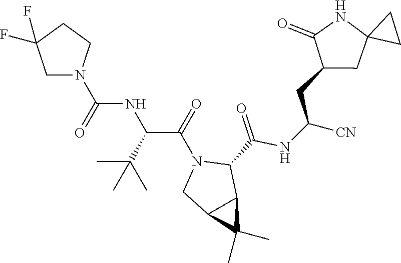 [SMILES: CC(C)(C)[C@H](NC(=O)N1CCC(F)(F)C1)C(=O)N1C[C@H]2[C@@H]([C@H]1C(=O)N[C@H](C#N)C[C@@H]1CC3(CC3)NC1=O)C2(C)C] |
| 241 | 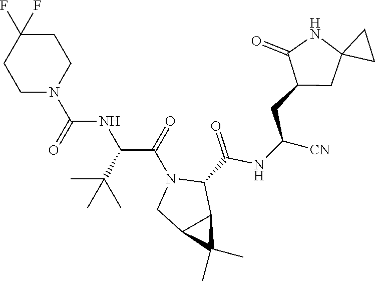 [SMILES: CC(C)(C)[C@H](NC(=O)N1CCC(F)(F)CC1)C(=O)N1C[C@H]2[C@@H]([C@H]1C(=O)N[C@H](C#N)C[C@@H]1CC3(CC3)NC1=O)C2(C)C] |
| 242 | 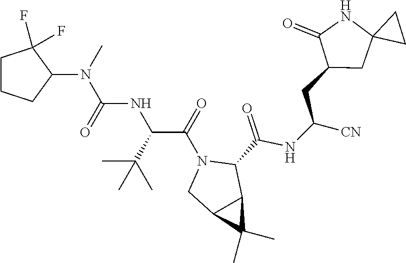 [SMILES: CN(C(=O)N[C@H](C(=O)N1C[C@H]2[C@@H]([C@H]1C(=O)N[C@H](C#N)C[C@@H]1CC3(CC3)NC1=O)C2(C)C)C(C)(C)C)C1CCCC1(F)F] |
| 243 | 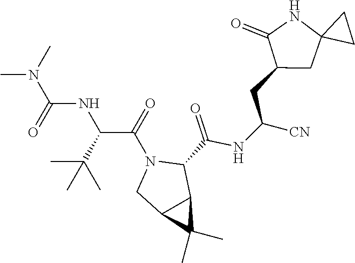 [SMILES: CN(C)C(=O)N[C@H](C(=O)N1C[C@H]2[C@@H]([C@H]1C(=O)N[C@H](C#N)C[C@@H]1CC3(CC3)NC1=O)C2(C)C)C(C)(C)C] |
| 244 | 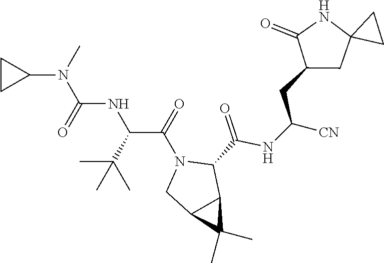 [SMILES: CN(C(=O)N[C@H](C(=O)N1C[C@H]2[C@@H]([C@H]1C(=O)N[C@H](C#N)C[C@@H]1CC3(CC3)NC1=O)C2(C)C)C(C)(C)C)C1CC1] |
| 245 | 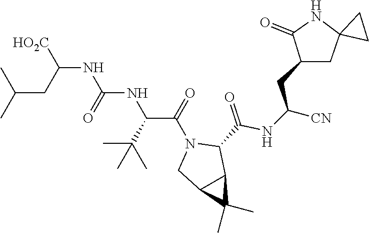 [SMILES: CC(C)CC(NC(=O)N[C@H](C(=O)N1C[C@H]2[C@@H]([C@H]1C(=O)N[C@H](C#N)C[C@@H]1CC3(CC3)NC1=O)C2(C)C)C(C)(C)C)C(=O)O] |
| 246 | 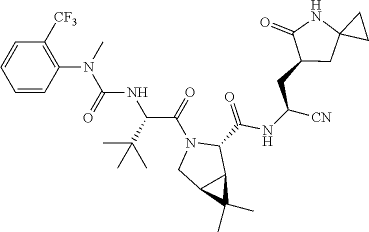 [SMILES: CN(C(=O)N[C@H](C(=O)N1C[C@H]2[C@@H]([C@H]1C(=O)N[C@H](C#N)C[C@@H]1CC3(CC3)NC1=O)C2(C)C)C(C)(C)C)c1ccccc1C(F)(F)F] |
| 247 | 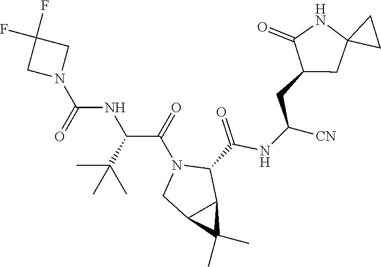 [SMILES: CC(C)(C)[C@H](NC(=O)N1CC(F)(F)C1)C(=O)N1C[C@H]2[C@@H]([C@H]1C(=O)N[C@H](C#N)C[C@@H]1CC3(CC3)NC1=O)C2(C)C] |
| 248 | 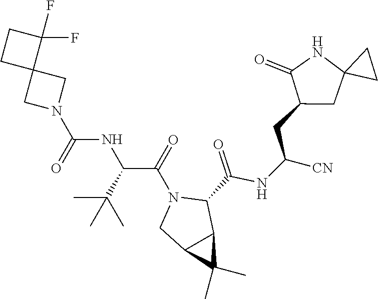 [SMILES: CC(C)(C)[C@H](NC(=O)N1CC2(CCC2(F)F)C1)C(=O)N1C[C@H]2[C@@H]([C@H]1C(=O)N[C@H](C#N)C[C@@H]1CC3(CC3)NC1=O)C2(C)C] |
| 249 | 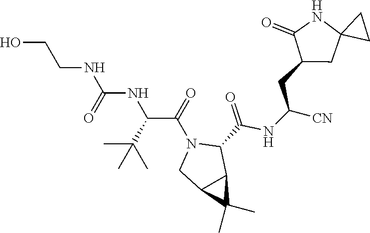 [SMILES: CC(C)(C)[C@H](NC(=O)NCCO)C(=O)N1C[C@H]2[C@@H]([C@H]1C(=O)N[C@H](C#N)C[C@@H]1CC3(CC3)NC1=O)C2(C)C] |
| 250 | 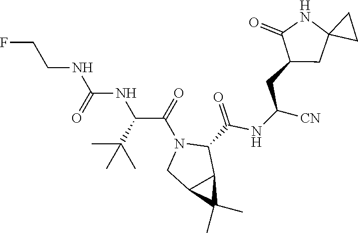 [SMILES: CC(C)(C)[C@H](NC(=O)NCCF)C(=O)N1C[C@H]2[C@@H]([C@H]1C(=O)N[C@H](C#N)C[C@@H]1CC3(CC3)NC1=O)C2(C)C] |
| 251 | 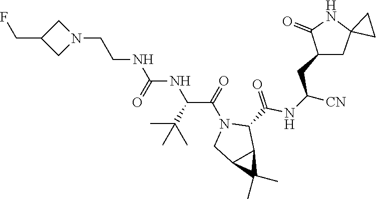 [SMILES: CC(C)(C)[C@H](NC(=O)NCCN1CC(CF)C1)C(=O)N1C[C@H]2[C@@H]([C@H]1C(=O)N[C@H](C#N)C[C@@H]1CC3(CC3)NC1=O)C2(C)C] |
| 252 | 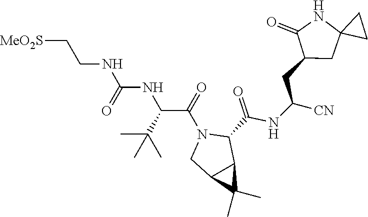 [SMILES: CC(C)(C)[C@H](NC(=O)NCCS(C)(=O)=O)C(=O)N1C[C@H]2[C@@H]([C@H]1C(=O)N[C@H](C#N)C[C@@H]1CC3(CC3)NC1=O)C2(C)C] |
| 253 | 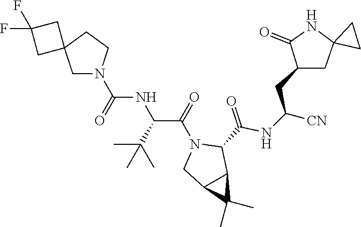 [SMILES: CC(C)(C)[C@H](NC(=O)N1CCC2(C1)CC(F)(F)C2)C(=O)N1C[C@H]2[C@@H]([C@H]1C(=O)N[C@H](C#N)C[C@@H]1CC3(CC3)NC1=O)C2(C)C] |
| 254 | 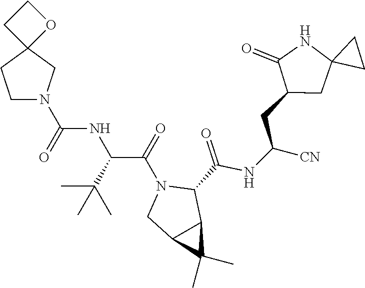 [SMILES: CC(C)(C)[C@H](NC(=O)N1CCC2(CCO2)C1)C(=O)N1C[C@H]2[C@@H]([C@H]1C(=O)N[C@H](C#N)C[C@@H]1CC3(CC3)NC1=O)C2(C)C] |
| 255 | 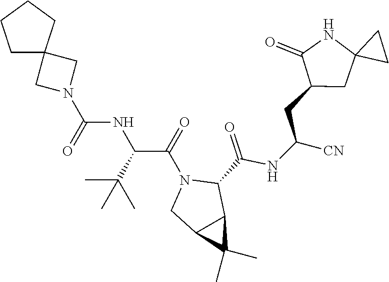 [SMILES: CC(C)(C)[C@H](NC(=O)N1CC2(CCCC2)C1)C(=O)N1C[C@H]2[C@@H]([C@H]1C(=O)N[C@H](C#N)C[C@@H]1CC3(CC3)NC1=O)C2(C)C] |
| 256 | 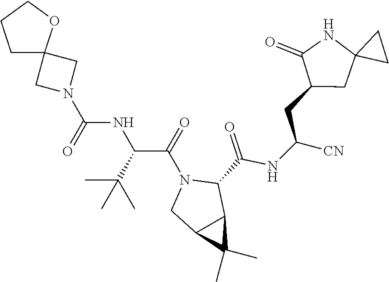 [SMILES: CC(C)(C)[C@H](NC(=O)N1CC2(CCCO2)C1)C(=O)N1C[C@H]2[C@@H]([C@H]1C(=O)N[C@H](C#N)C[C@@H]1CC3(CC3)NC1=O)C2(C)C] |
| 257 | 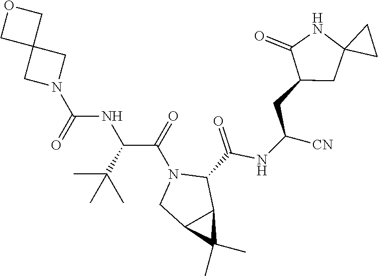 [SMILES: CC(C)(C)[C@H](NC(=O)N1CC2(COC2)C1)C(=O)N1C[C@H]2[C@@H]([C@H]1C(=O)N[C@H](C#N)C[C@@H]1CC3(CC3)NC1=O)C2(C)C] |
| 258 | 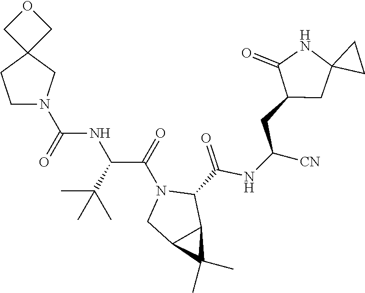 [SMILES: CC(C)(C)[C@H](NC(=O)N1CCC2(COC2)C1)C(=O)N1C[C@H]2[C@@H]([C@H]1C(=O)N[C@H](C#N)C[C@@H]1CC3(CC3)NC1=O)C2(C)C] |
| 259 | 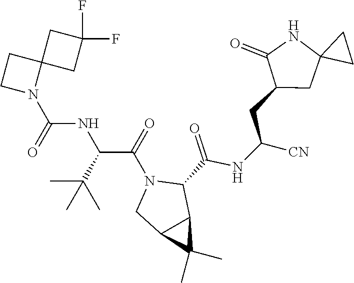 [SMILES: CC(C)(C)[C@H](NC(=O)N1CCC12CC(F)(F)C2)C(=O)N1C[C@H]2[C@@H]([C@H]1C(=O)N[C@H](C#N)C[C@@H]1CC3(CC3)NC1=O)C2(C)C] |
| 260 | 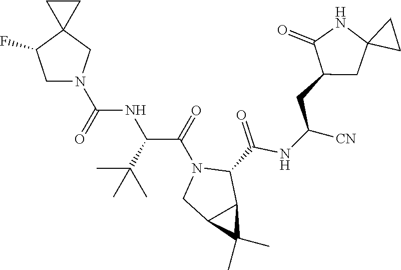 [SMILES: CC(C)(C)[C@H](NC(=O)N1C[C@H](F)C2(CC2)C1)C(=O)N1C[C@H]2[C@@H]([C@H]1C(=O)N[C@H](C#N)C[C@@H]1CC3(CC3)NC1=O)C2(C)C] |
| 261 | 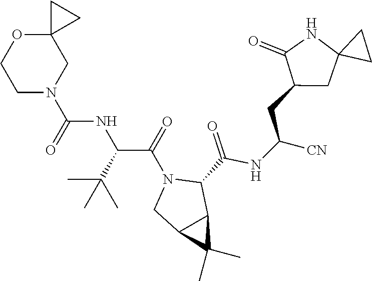 [SMILES: CC(C)(C)[C@H](NC(=O)N1CCOC2(CC2)C1)C(=O)N1C[C@H]2[C@@H]([C@H]1C(=O)N[C@H](C#N)C[C@@H]1CC3(CC3)NC1=O)C2(C)C] |
| 262 | 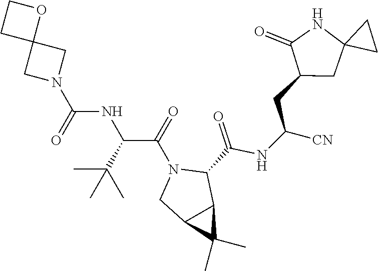 [SMILES: CC(C)(C)[C@H](NC(=O)N1CC2(CCO2)C1)C(=O)N1C[C@H]2[C@@H]([C@H]1C(=O)N[C@H](C#N)C[C@@H]1CC3(CC3)NC1=O)C2(C)C] |

In embodiments, a disclosed compound is selected from the group consisting of

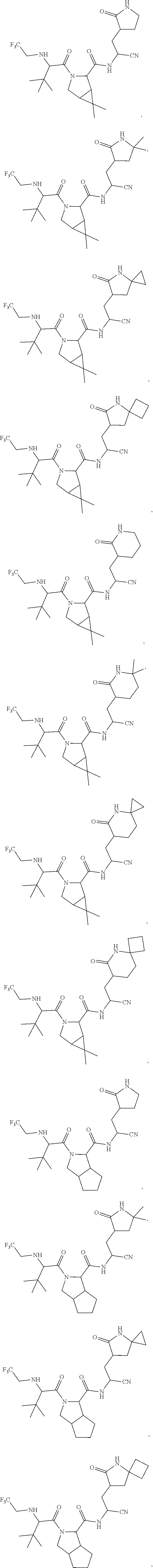

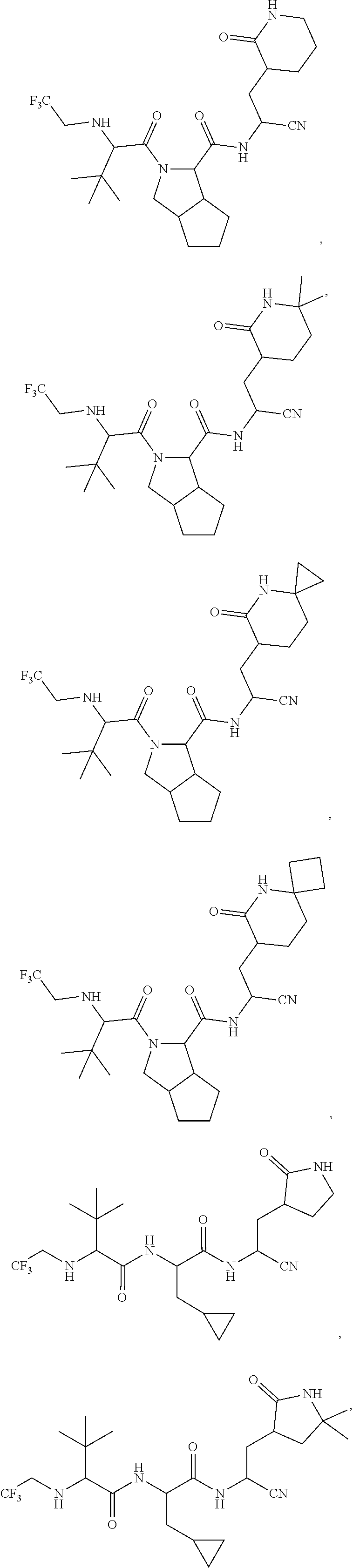

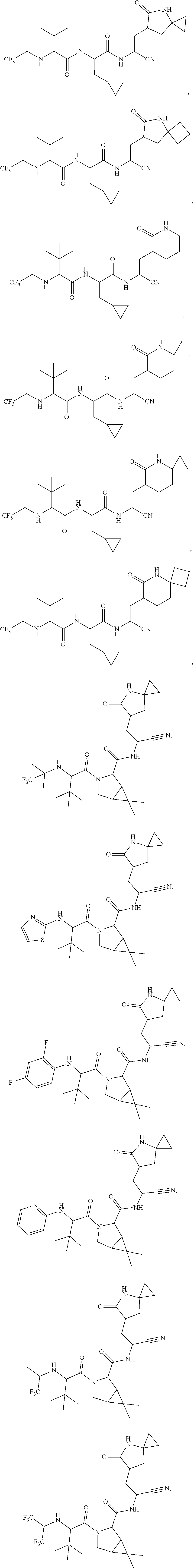

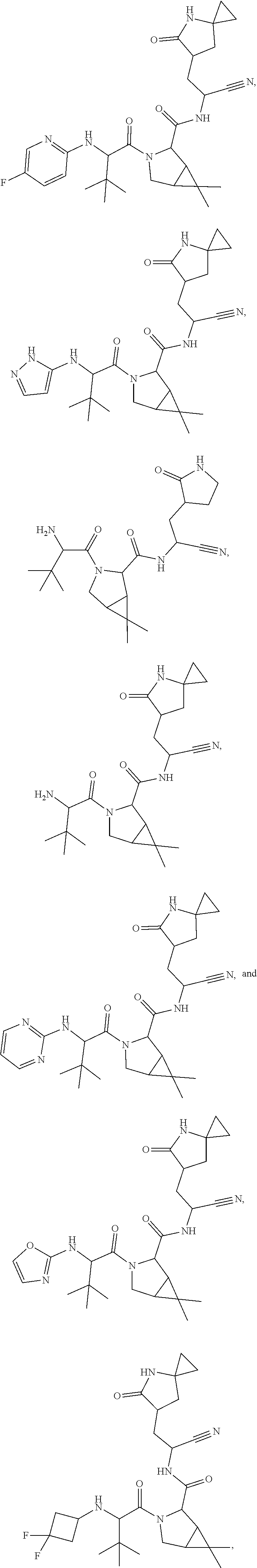

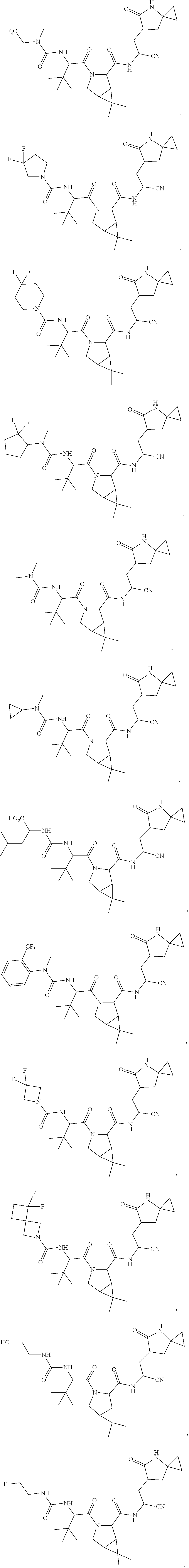

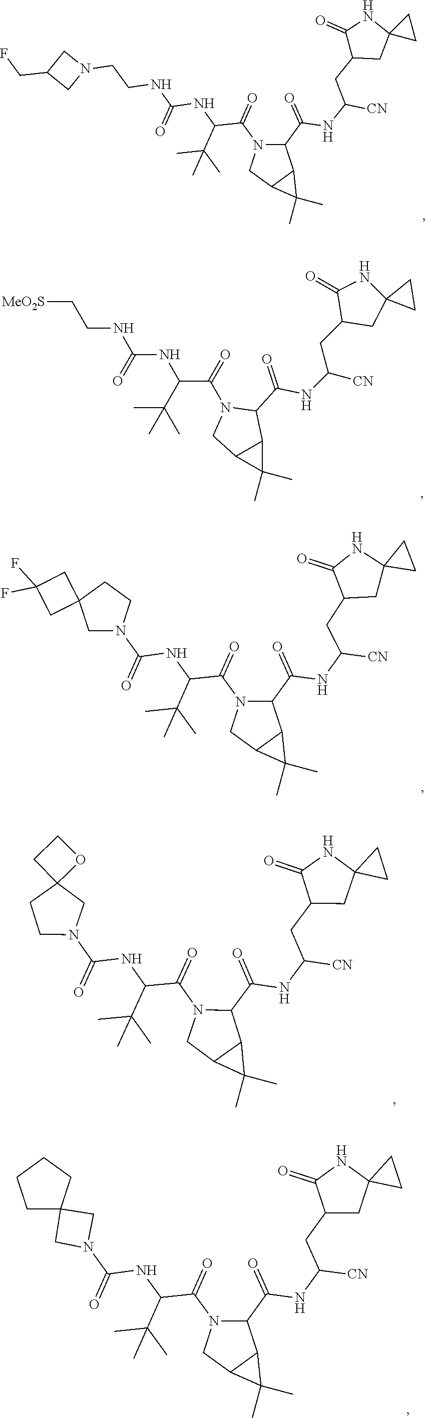 [SMILES: CC(C)(C)C(NC(=O)NCCN1CC(CF)C1)C(=O)N1CC2C(C1C(=O)NC(C#N)CC1CC3(CC3)NC1=O)C2(C)C ; CC(C)(C)C(NC(=O)NCCS(C)(=O)=O)C(=O)N1CC2C(C1C(=O)NC(C#N)CC1CC3(CC3)NC1=O)C2(C)C ; CC(C)(C)C(NC(=O)N1CCC2(C1)CC(F)(F)C2)C(=O)N1CC2C(C1C(=O)NC(C#N)CC1CC3(CC3)NC1=O)C2(C)C ; CC(C)(C)C(NC(=O)N1CCC2(CCO2)C1)C(=O)N1CC2C(C1C(=O)NC(C#N)CC1CC3(CC3)NC1=O)C2(C)C ; CC(C)(C)C(NC(=O)N1CC2(CCCC2)C1)C(=O)N1CC2C(C1C(=O)NC(C#N)CC1CC3(CC3)NC1=O)C2(C)C]

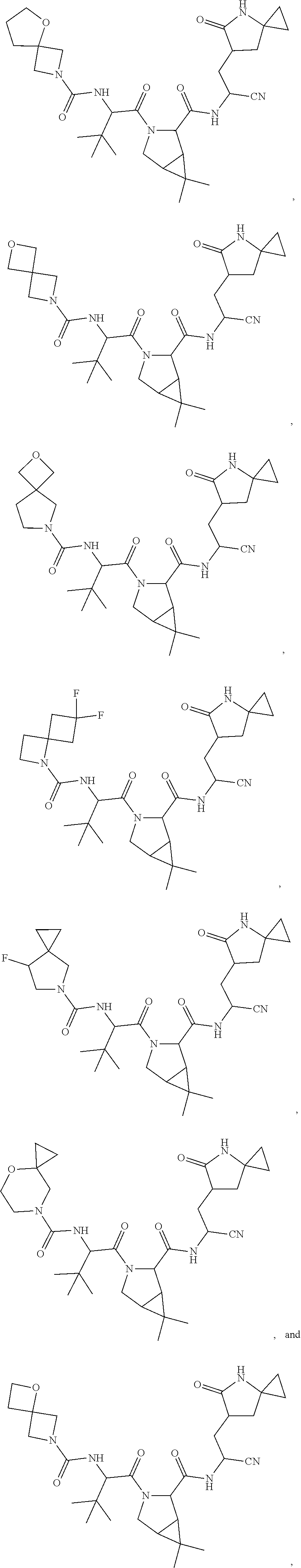

or a pharmaceutically acceptable salt thereof.

Also disclosed herein is a protease inhibitory compound represented by Formula III:

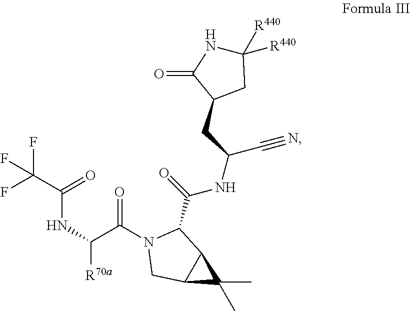

or a pharmaceutically acceptable salt or stereoisomer thereof, wherein R⁴⁰⁰ is each independently selected from the group consisting of hydrogen, —CH₃ and —CF₃, or two R¹ groups, together with the carbon to which they are attached, may be joined together to form a cyclopropyl, cyclobutyl, or oxetanyl; R^{70a} is selected from the group consisting of C₁₋₃alkylene-C₃-C₅cycloalkyl, C₁₋₆alkyl, C₃-C₅cycloalkyl, C₁₋₃alkyl-O— R⁵⁰¹ and a 4-6 membered heterocyclyl having one ring oxygen; wherein R^{70a} may optionally be substituted by one, two, three substituents each independently selected from R⁵⁰⁰; R⁵⁰¹ is selected from the group consisting of C₁₋₆alkyl, C₁₋₆alkenyl, phenyl, C₃-C₆cycloalkyl, 4-6 membered heterocyclyl having one ring oxygen and 5-6 membered heteroaryl, wherein R⁵⁰⁰ may be optionally substituted by one two, three or more substituents each independently selected from the group consisting of halo, cyano, methyl, ethyl, C₁₋₆alkoxy, CF₃, —CHF₂—CH₂—CF₃ and C₃₋₆cycloalkyl; and R⁵⁰⁰ is independently selected for each occurrence from the group consisting of halogen, cyano, —CF₃, —CHF₂, C₁-C₂alkyl, and C₂₋₆alkenyl.

Also disclosed herein is a protease inhibitory compound represented by Formula

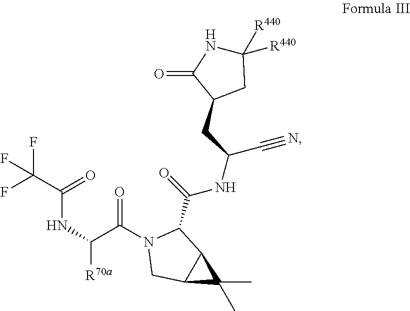

or a pharmaceutically acceptable salt or stereoisomer thereof, wherein R⁴⁴⁰ is each independently selected from the group consisting of hydrogen, —CH₃ and —CF₃, or two R¹ groups, together with the carbon to which they are attached, may be joined together to form a cyclopropyl, cyclobutyl, or oxetanyl; R^{70a} is selected from the group consisting of C₁₋₃alkylene-C₃-C₅cycloalkyl, C₃-C₅cycloalkyl, C₁₋₃alkyl-O— R⁵⁰¹ and a 4-6 membered heterocyclyl having one ring oxygen; wherein R^{70a} may optionally be substituted by one, two, three substituents each independently selected from R⁵⁰⁰; R⁵⁰¹ is selected from the group consisting of C₁₋₄alkyl, phenyl, C₃-C₅cycloalkyl, 4-6 membered heterocyclyl having one ring oxygen and (5-6 membered heteroaryl) wherein R⁵⁰¹ may be optionally substituted by one two, three or more substituents each independently selected from the group consisting of halo, cyano, methyl, ethyl, CF₃, —CHF₂, —CH₂—CF₃ and C₃cycloalkyl; and R⁵⁰⁰ is independently selected for each occurrence from the group consisting of halogen, cyano, —CF₃, —CHF₂, and C₁-C₂alkyl.

Also disclosed herein is a protease inhibitory compound represented by Formula IV:

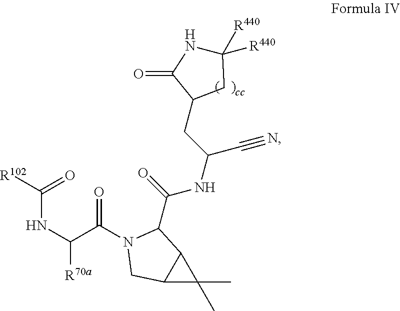

or a pharmaceutically acceptable salt or stereoisomer thereof, wherein R⁴⁰⁰ is each independently selected from the group consisting of hydrogen, —CH₃ and —CF₃, or two R⁴⁴⁰ groups, together with the carbon to which they are attached, may be joined together to form a cyclopropyl, cyclobutyl, or oxetanyl; R^{70a} is selected from the group consisting of C₁₋₃alkylene-C₃-C₅cycloalkyl, C₁₋₆alkyl, C₃-05cycloalkyl, C₁-3alkyl-O— R⁵⁰¹ and a 4-6 membered heterocyclyl having one ring oxygen; wherein R^{70a} may optionally be substituted by one, two, three substituents each independently selected from R⁵⁰⁰; R⁵⁰¹ is selected from the group consisting of C₁₋₆alkyl, C₁₋₆alkenyl, phenyl, C₃-C₆cycloalkyl, 4-6 membered heterocyclyl having one ring oxygen and (5-6 membered heteroaryl) wherein R⁵⁰¹ may be optionally substituted by one two, three or more substituents each independently selected from the group consisting of halo, cyano, methyl, ethyl, C₁₋₆alkoxy, CF₃, —CHF₂. —CH₂—CF₃ and C₃₋₆cycloalkyl; R⁵⁰⁰ is independently selected for each occurrence from the group consisting of halogen, cyano, —CF₃, —CHF₂, C₁-C₂alkyl, and C₂₋₆alkenyl; and R¹⁰² is selected from —NR^{m}(C═O)R¹¹ and

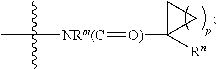

p is selected from 1 and 2; R^{m} is H; R is selected from C₁₋₂alkyl and C₃-C₄cycloalkyl, wherein the alkyl or cycloalkyl is optionally substituted by one, two or three halo or —CF₃; and cc is selected from 1 and 2.

In some embodiments, Formula N is represented by Formula Ni:

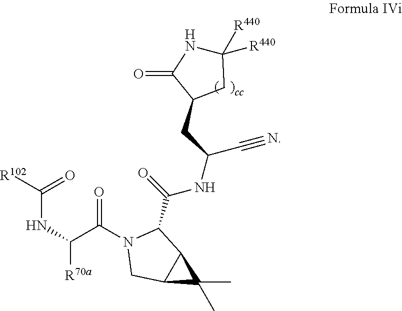

In some embodiments, a disclosed compound is selected from the group consisting of the compounds identified in Table 2a, or pharmaceutically acceptable salt thereof.

TABLE 2a

| Cmpd | |
|---|---|
| No. | Structure |
| 263 | 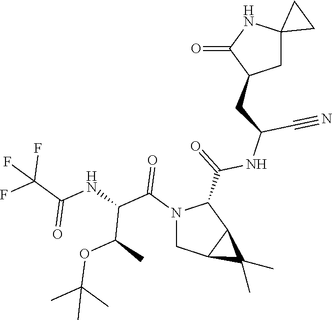 [SMILES: C[C@@H](OC(C)(C)C)[C@H](NC(=O)C(F)(F)F)C(=O)N1C[C@H]2[C@@H]([C@H]1C(=O)N[C@H](C#N)C[C@@H]1CC3(CC3)NC1=O)C2(C)C] |
| 264 | 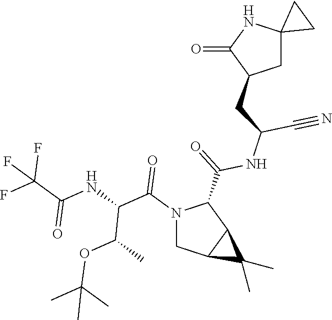 [SMILES: C[C@H](OC(C)(C)C)[C@H](NC(=O)C(F)(F)F)C(=O)N1C[C@H]2[C@@H]([C@H]1C(=O)N[C@H](C#N)C[C@@H]1CC3(CC3)NC1=O)C2(C)C] |
| 266 | 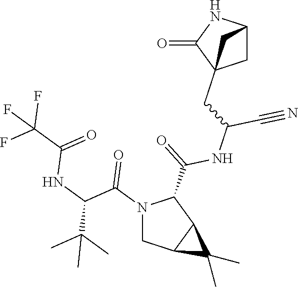 [SMILES: CC(C)(C)[C@H](NC(=O)C(F)(F)F)C(=O)N1C[C@H]2[C@@H]([C@H]1C(=O)NC(C#N)C[C@]13C[C@H](C1)NC3=O)C2(C)C] |
| 267 | 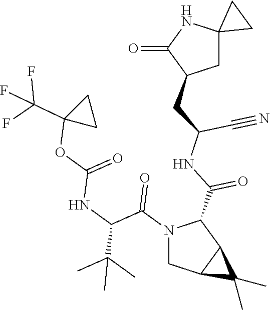 [SMILES: CC(C)(C)[C@H](NC(=O)OC1(C(F)(F)F)CC1)C(=O)N1C[C@H]2[C@@H]([C@H]1C(=O)N[C@H](C#N)C[C@@H]1CC3(CC3)NC1=O)C2(C)C] |
| 268 | 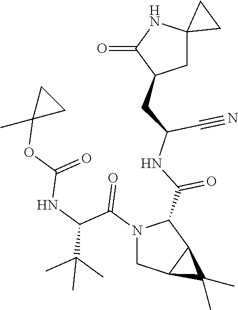 |
| 269 | 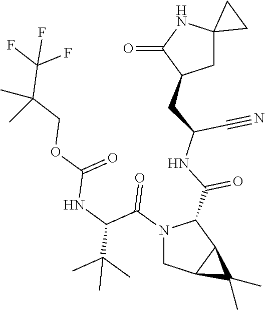 [SMILES: CC(C)(C)[C@H](NC(=O)OCC(C)(C)C(F)(F)F)C(=O)N1C[C@H]2[C@@H]([C@H]1C(=O)N[C@H](C#N)C[C@@H]1CC3(CC3)NC1=O)C2(C)C] |
| 270 | 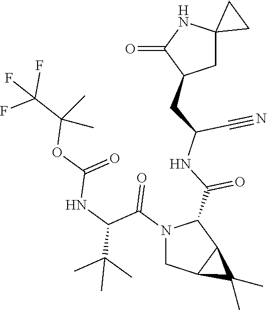 [SMILES: CC(C)(C)[C@H](NC(=O)OC(C)(C)C(F)(F)F)C(=O)N1C[C@H]2[C@@H]([C@H]1C(=O)N[C@H](C#N)C[C@@H]1CC3(CC3)NC1=O)C2(C)C] |
| 271 | 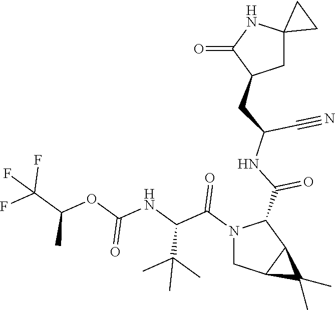 [SMILES: C[C@H](OC(=O)N[C@H](C(=O)N1C[C@H]2[C@@H]([C@H]1C(=O)N[C@H](C#N)C[C@@H]1CC3(CC3)NC1=O)C2(C)C)C(C)(C)C)C(F)(F)F] |
| 272 | 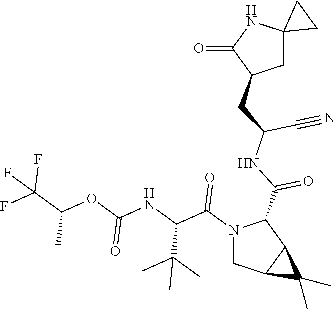 [SMILES: C[C@@H](OC(=O)N[C@H](C(=O)N1C[C@H]2[C@@H]([C@H]1C(=O)N[C@H](C#N)C[C@@H]1CC3(CC3)NC1=O)C2(C)C)C(C)(C)C)C(F)(F)F] |
| 273 | 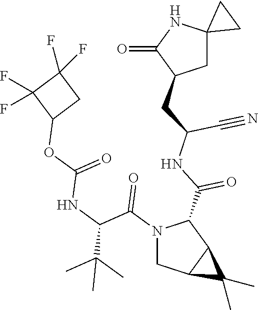 [SMILES: CC(C)(C)[C@H](NC(=O)OC1CC(F)(F)C1(F)F)C(=O)N1C[C@H]2[C@@H]([C@H]1C(=O)N[C@H](C#N)C[C@@H]1CC3(CC3)NC1=O)C2(C)C] |
| 274 | 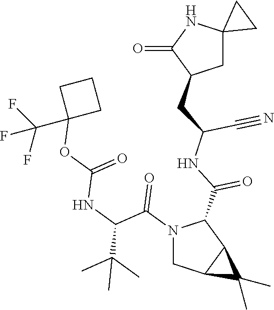 [SMILES: CC(C)(C)[C@H](NC(=O)OC1(C(F)(F)F)CCC1)C(=O)N1C[C@H]2[C@@H]([C@H]1C(=O)N[C@H](C#N)C[C@@H]1CC3(CC3)NC1=O)C2(C)C] |
| 275 | 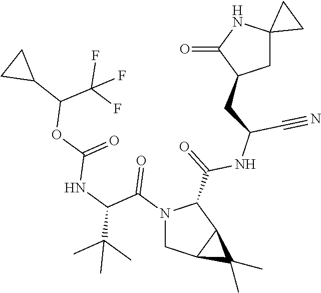 [SMILES: CC(C)(C)[C@H](NC(=O)OC(C1CC1)C(F)(F)F)C(=O)N1C[C@H]2[C@@H]([C@H]1C(=O)N[C@H](C#N)C[C@@H]1CC3(CC3)NC1=O)C2(C)C] |
| 276 | 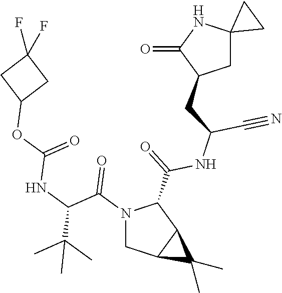 [SMILES: CC(C)(C)[C@H](NC(=O)OC1CC(F)(F)C1)C(=O)N1C[C@H]2[C@@H]([C@H]1C(=O)N[C@H](C#N)C[C@@H]1CC3(CC3)NC1=O)C2(C)C] |
| 277 | 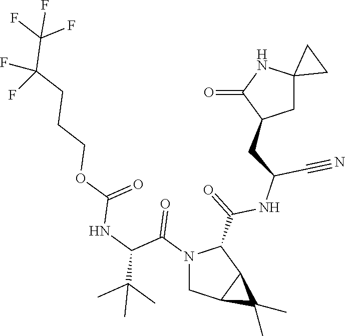 [SMILES: CC(C)(C)[C@H](NC(=O)OCCCC(F)(F)C(F)(F)F)C(=O)N1C[C@H]2[C@@H]([C@H]1C(=O)N[C@H](C#N)C[C@@H]1CC3(CC3)NC1=O)C2(C)C] |
| 278 | 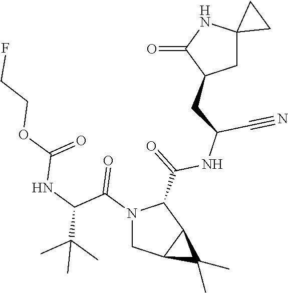 [SMILES: CC(C)(C)[C@H](NC(=O)OCCF)C(=O)N1C[C@H]2[C@@H]([C@H]1C(=O)N[C@H](C#N)C[C@@H]1CC3(CC3)NC1=O)C2(C)C] |
| 279 | 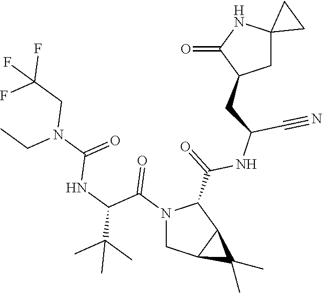 [SMILES: CCN(CC(F)(F)F)C(=O)N[C@H](C(=O)N1C[C@H]2[C@@H]([C@H]1C(=O)N[C@H](C#N)C[C@@H]1CC3(CC3)NC1=O)C2(C)C)C(C)(C)C] |
| 280 | 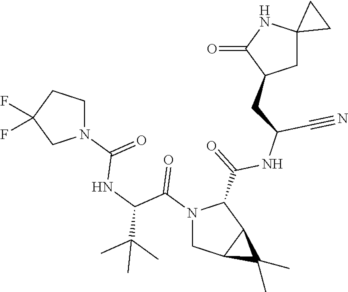 [SMILES: CC(C)(C)[C@H](NC(=O)N1CCC(F)(F)C1)C(=O)N1C[C@H]2[C@@H]([C@H]1C(=O)N[C@H](C#N)C[C@@H]1CC3(CC3)NC1=O)C2(C)C] |
| 281 | 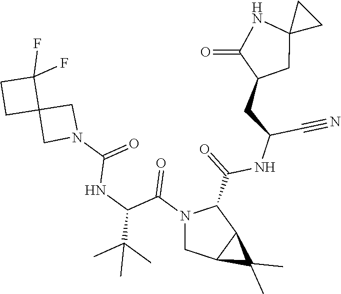 [SMILES: CC(C)(C)[C@H](NC(=O)N1CC2(CCC2(F)F)C1)C(=O)N1C[C@H]2[C@@H]([C@H]1C(=O)N[C@H](C#N)C[C@@H]1CC3(CC3)NC1=O)C2(C)C] |
| 282 | 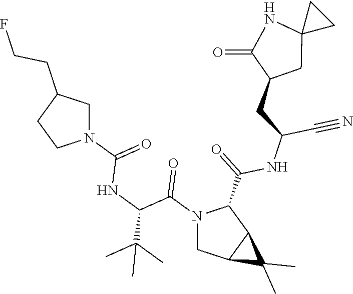 [SMILES: CC(C)(C)[C@H](NC(=O)N1CCC(CCF)C1)C(=O)N1C[C@H]2[C@@H]([C@H]1C(=O)N[C@H](C#N)C[C@@H]1CC3(CC3)NC1=O)C2(C)C] |
| 283 | 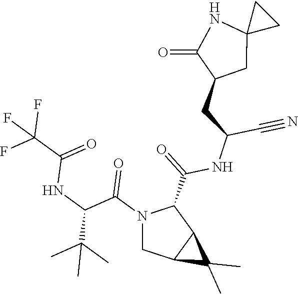 [SMILES: CC(C)(C)[C@H](NC(=O)C(F)(F)F)C(=O)N1C[C@H]2[C@@H]([C@H]1C(=O)N[C@H](C#N)C[C@@H]1CC3(CC3)NC1=O)C2(C)C] |
| 284 | 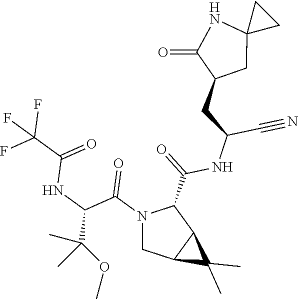 [SMILES: COC(C)(C)[C@H](NC(=O)C(F)(F)F)C(=O)N1C[C@H]2[C@@H]([C@H]1C(=O)N[C@H](C#N)C[C@@H]1CC3(CC3)NC1=O)C2(C)C] |
| 285 | 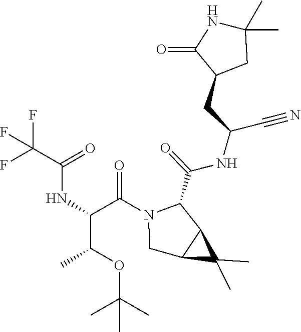 [SMILES: C[C@@H](OC(C)(C)C)[C@H](NC(=O)C(F)(F)F)C(=O)N1C[C@H]2[C@@H]([C@H]1C(=O)N[C@H](C#N)C[C@@H]1CC(C)(C)NC1=O)C2(C)C] |
| 286 | 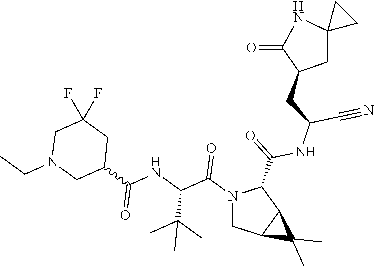 [SMILES: CCN1CC(C(=O)N[C@H](C(=O)N2C[C@H]3[C@@H]([C@H]2C(=O)N[C@H](C#N)C[C@@H]2CC4(CC4)NC2=O)C3(C)C)C(C)(C)C)CC(F)(F)C1] |
| 287 | 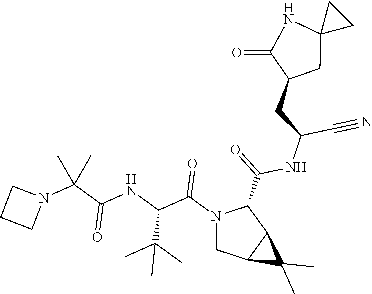 [SMILES: CC(C)(C)[C@H](NC(=O)C(C)(C)N1CCC1)C(=O)N1C[C@H]2[C@@H]([C@H]1C(=O)N[C@H](C#N)C[C@@H]1CC3(CC3)NC1=O)C2(C)C] |
| 288 | 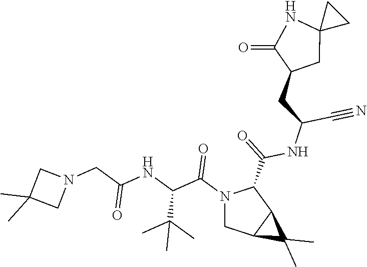 [SMILES: CC1(C)CN(CC(=O)N[C@H](C(=O)N2C[C@H]3[C@@H]([C@H]2C(=O)N[C@H](C#N)C[C@@H]2CC4(CC4)NC2=O)C3(C)C)C(C)(C)C)C1] |
| 289 | 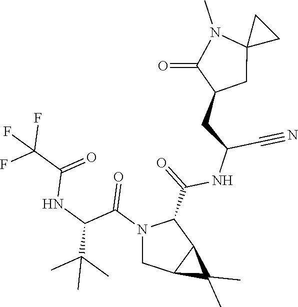 [SMILES: CN1C(=O)[C@H](C[C@@H](C#N)NC(=O)[C@@H]2[C@@H]3[C@H](CN2C(=O)[C@@H](NC(=O)C(F)(F)F)C(C)(C)C)C3(C)C)CC12CC2] |
| 290 | 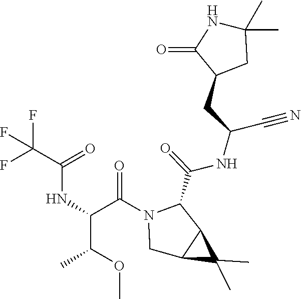 [SMILES: CO[C@H](C)[C@H](NC(=O)C(F)(F)F)C(=O)N1C[C@H]2[C@@H]([C@H]1C(=O)N[C@H](C#N)C[C@@H]1CC(C)(C)NC1=O)C2(C)C] |
| 291 | 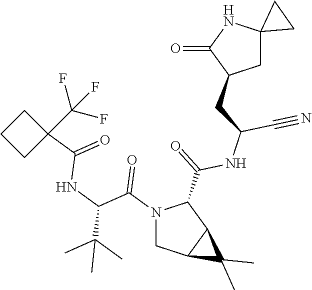 [SMILES: CC(C)(C)[C@H](NC(=O)C1(C(F)(F)F)CCC1)C(=O)N1C[C@H]2[C@@H]([C@H]1C(=O)N[C@H](C#N)C[C@@H]1CC3(CC3)NC1=O)C2(C)C] |
| 292 | 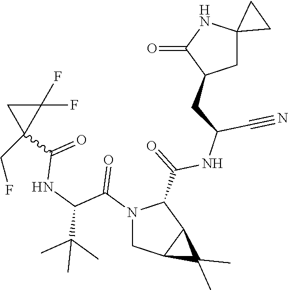 [SMILES: CC(C)(C)[C@H](NC(=O)C1(CF)CC1(F)F)C(=O)N1C[C@H]2[C@@H]([C@H]1C(=O)N[C@H](C#N)C[C@@H]1CC3(CC3)NC1=O)C2(C)C] |
| 293 | 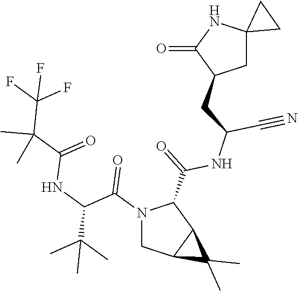 [SMILES: CC(C)(C)[C@H](NC(=O)C(C)(C)C(F)(F)F)C(=O)N1C[C@H]2[C@@H]([C@H]1C(=O)N[C@H](C#N)C[C@@H]1CC3(CC3)NC1=O)C2(C)C] |
| 294 | 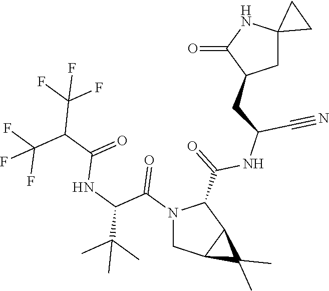 [SMILES: CC(C)(C)[C@H](NC(=O)C(C(F)(F)F)C(F)(F)F)C(=O)N1C[C@H]2[C@@H]([C@H]1C(=O)N[C@H](C#N)C[C@@H]1CC3(CC3)NC1=O)C2(C)C] |
| 295 | 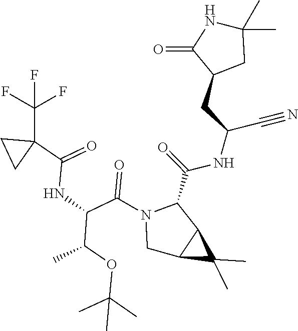 [SMILES: C[C@@H](OC(C)(C)C)[C@H](NC(=O)C1(C(F)(F)F)CC1)C(=O)N1C[C@H]2[C@@H]([C@H]1C(=O)N[C@H](C#N)C[C@@H]1CC(C)(C)NC1=O)C2(C)C] |
| 296 | 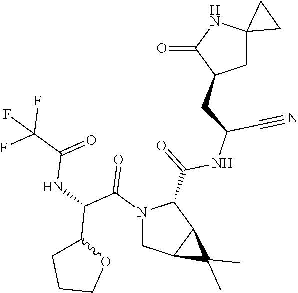 [SMILES: CC1(C)[C@@H]2[C@@H](C(=O)N[C@H](C#N)C[C@@H]3CC4(CC4)NC3=O)N(C(=O)[C@@H](NC(=O)C(F)(F)F)C3CCCO3)C[C@@H]21] |
| 297 | 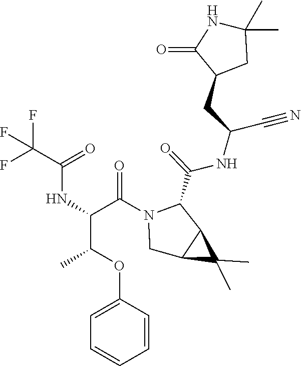 [SMILES: C[C@@H](Oc1ccccc1)[C@H](NC(=O)C(F)(F)F)C(=O)N1C[C@H]2[C@@H]([C@H]1C(=O)N[C@H](C#N)C[C@@H]1CC(C)(C)NC1=O)C2(C)C] |
| 298 | 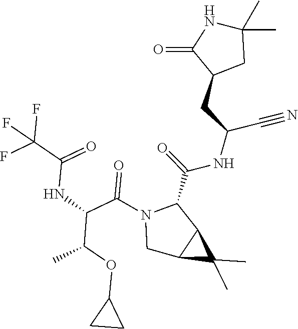 [SMILES: C[C@@H](OC1CC1)[C@H](NC(=O)C(F)(F)F)C(=O)N1C[C@H]2[C@@H]([C@H]1C(=O)N[C@H](C#N)C[C@@H]1CC(C)(C)NC1=O)C2(C)C] |
| 298a | 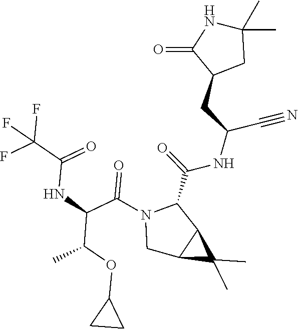 [SMILES: C[C@@H](OC1CC1)[C@@H](NC(=O)C(F)(F)F)C(=O)N1C[C@H]2[C@@H]([C@H]1C(=O)N[C@H](C#N)C[C@@H]1CC(C)(C)NC1=O)C2(C)C] |
| 299 | 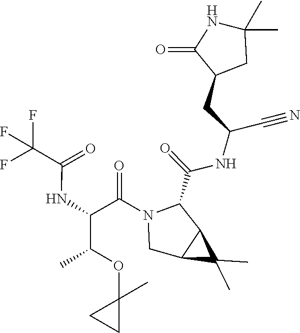 [SMILES: C[C@@H](OC1(C)CC1)[C@H](NC(=O)C(F)(F)F)C(=O)N1C[C@H]2[C@@H]([C@H]1C(=O)N[C@H](C#N)C[C@@H]1CC(C)(C)NC1=O)C2(C)C] |
| 299a | 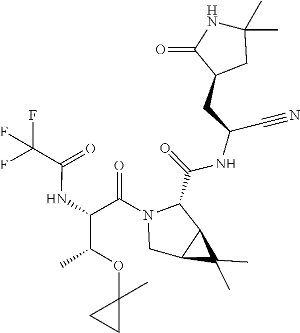 [SMILES: C[C@@H](OC1(C)CC1)[C@H](NC(=O)C(F)(F)F)C(=O)N1C[C@H]2[C@@H]([C@H]1C(=O)N[C@H](C#N)C[C@@H]1CC(C)(C)NC1=O)C2(C)C] |
| 300 | 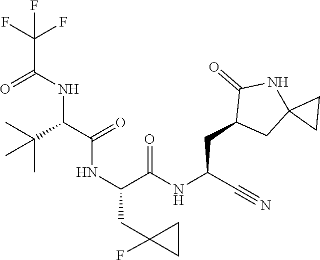 [SMILES: CC(C)(C)[C@H](NC(=O)C(F)(F)F)C(=O)N[C@@H](CC1(F)CC1)C(=O)N[C@H](C#N)C[C@@H]1CC2(CC2)NC1=O] |
| 301 | 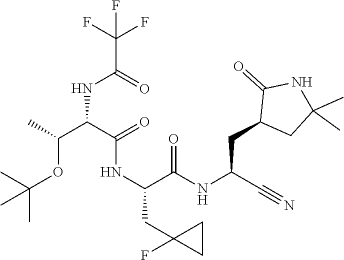 [SMILES: C[C@@H](OC(C)(C)C)[C@H](NC(=O)C(F)(F)F)C(=O)N[C@@H](CC1(F)CC1)C(=O)N[C@H](C#N)C[C@@H]1CC(C)(C)NC1=O] |
| 301a | 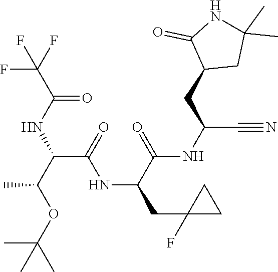 [SMILES: C[C@@H](OC(C)(C)C)[C@H](NC(=O)C(F)(F)F)C(=O)N[C@H](CC1(F)CC1)C(=O)N[C@H](C#N)C[C@@H]1CC(C)(C)NC1=O] |
| 302 | 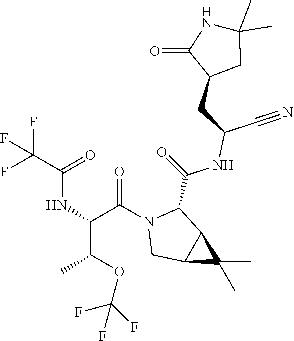 [SMILES: C[C@@H](OC(F)(F)F)[C@H](NC(=O)C(F)(F)F)C(=O)N1C[C@H]2[C@@H]([C@H]1C(=O)N[C@H](C#N)C[C@@H]1CC(C)(C)NC1=O)C2(C)C] |
| 303 | 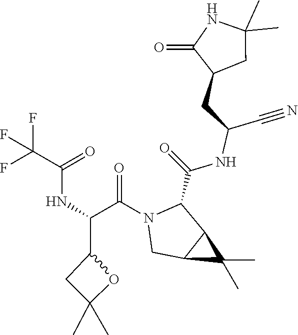 [SMILES: CC1(C)C[C@@H](C[C@@H](C#N)NC(=O)[C@@H]2[C@@H]3[C@H](CN2C(=O)[C@@H](NC(=O)C(F)(F)F)C2CC(C)(C)O2)C3(C)C)C(=O)N1] |
| 304 | 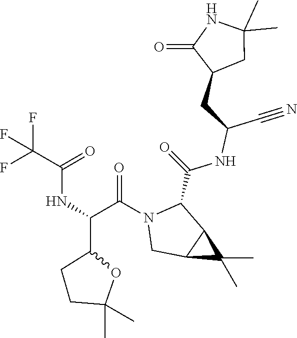 [SMILES: CC1(C)C[C@@H](C[C@@H](C#N)NC(=O)[C@@H]2[C@@H]3[C@H](CN2C(=O)[C@@H](NC(=O)C(F)(F)F)C2CCC(C)(C)O2)C3(C)C)C(=O)N1] |
| 305 | 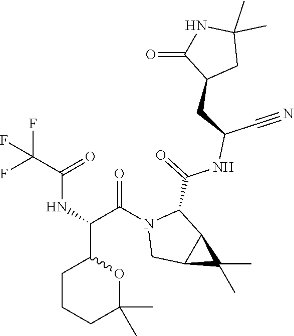 [SMILES: CC1(C)C[C@@H](C[C@@H](C#N)NC(=O)[C@@H]2[C@@H]3[C@H](CN2C(=O)[C@@H](NC(=O)C(F)(F)F)C2CCCC(C)(C)O2)C3(C)C)C(=O)N1] |
| 306 | 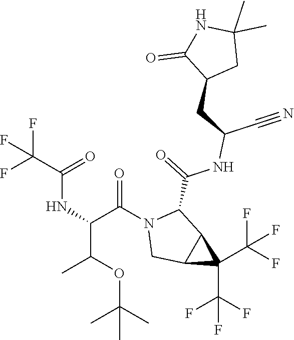 [SMILES: CC(OC(C)(C)C)[C@H](NC(=O)C(F)(F)F)C(=O)N1C[C@H]2[C@@H]([C@H]1C(=O)N[C@H](C#N)C[C@@H]1CC(C)(C)NC1=O)C2(C(F)(F)F)C(F)(F)F] |
| 307 | 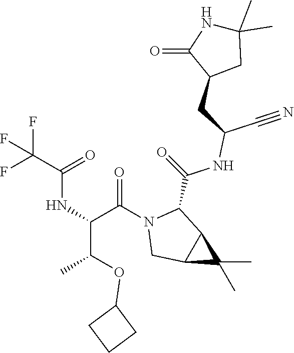 [SMILES: C[C@@H](OC1CCC1)[C@H](NC(=O)C(F)(F)F)C(=O)N1C[C@H]2[C@@H]([C@H]1C(=O)N[C@H](C#N)C[C@@H]1CC(C)(C)NC1=O)C2(C)C] |
| 308 | 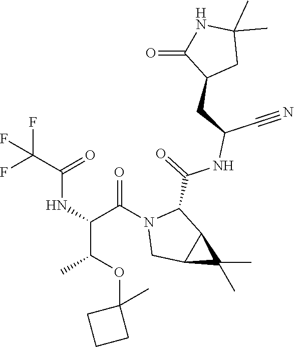 [SMILES: C[C@@H](OC1(C)CCC1)[C@H](NC(=O)C(F)(F)F)C(=O)N1C[C@H]2[C@@H]([C@H]1C(=O)N[C@H](C#N)C[C@@H]1CC(C)(C)NC1=O)C2(C)C] |
| 309 | 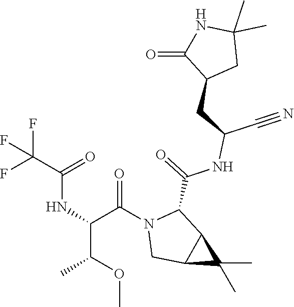 [SMILES: CO[C@H](C)[C@H](NC(=O)C(F)(F)F)C(=O)N1C[C@H]2[C@@H]([C@H]1C(=O)N[C@H](C#N)C[C@@H]1CC(C)(C)NC1=O)C2(C)C] |
| 310 | 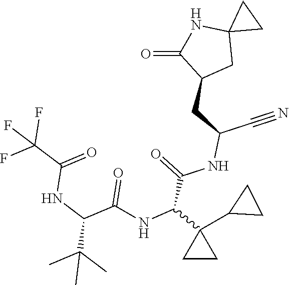 |
| 311 | 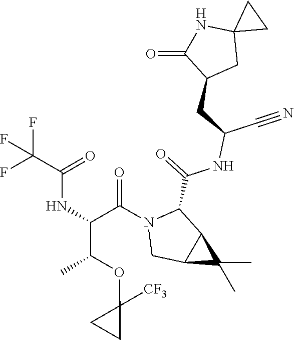 [SMILES: C[C@@H](OC1(C(F)(F)F)CC1)[C@H](NC(=O)C(F)(F)F)C(=O)N1C[C@H]2[C@@H]([C@H]1C(=O)N[C@H](C#N)C[C@@H]1CC3(CC3)NC1=O)C2(C)C] |
| 312 | 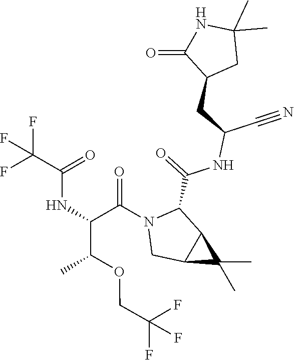 [SMILES: C[C@@H](OCC(F)(F)F)[C@H](NC(=O)C(F)(F)F)C(=O)N1C[C@H]2[C@@H]([C@H]1C(=O)N[C@H](C#N)C[C@@H]1CC(C)(C)NC1=O)C2(C)C] |
| 313 | 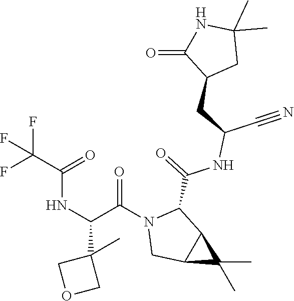 [SMILES: CC1(C)C[C@@H](C[C@@H](C#N)NC(=O)[C@@H]2[C@@H]3[C@H](CN2C(=O)[C@@H](NC(=O)C(F)(F)F)C2(C)COC2)C3(C)C)C(=O)N1] |
| 314 | 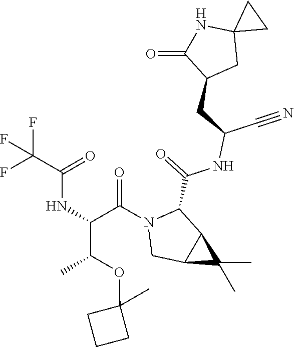 [SMILES: C[C@@H](OC1(C)CCC1)[C@H](NC(=O)C(F)(F)F)C(=O)N1C[C@H]2[C@@H]([C@H]1C(=O)N[C@H](C#N)C[C@@H]1CC3(CC3)NC1=O)C2(C)C] |
| 315 | 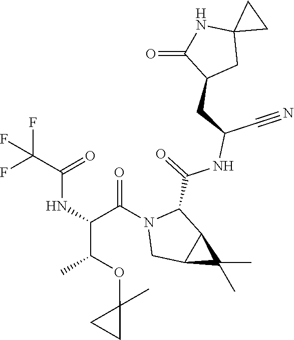 [SMILES: C[C@@H](OC1(C)CC1)[C@H](NC(=O)C(F)(F)F)C(=O)N1C[C@H]2[C@@H]([C@H]1C(=O)N[C@H](C#N)C[C@@H]1CC3(CC3)NC1=O)C2(C)C] |
| 316 | 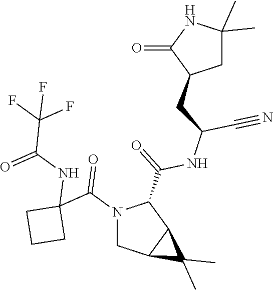 [SMILES: CC1(C)C[C@@H](C[C@@H](C#N)NC(=O)[C@@H]2[C@@H]3[C@H](CN2C(=O)C2(NC(=O)C(F)(F)F)CCC2)C3(C)C)C(=O)N1] |
| 317 | 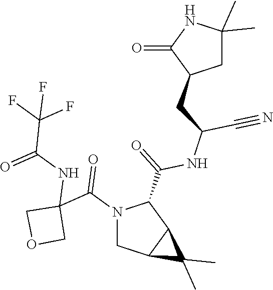 [SMILES: CC1(C)C[C@@H](C[C@@H](C#N)NC(=O)[C@@H]2[C@@H]3[C@H](CN2C(=O)C2(NC(=O)C(F)(F)F)COC2)C3(C)C)C(=O)N1] |
| 318 | 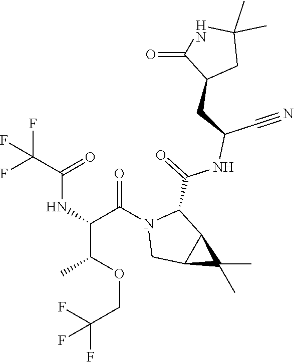 [SMILES: C[C@@H](OCC(F)(F)F)[C@H](NC(=O)C(F)(F)F)C(=O)N1C[C@H]2[C@@H]([C@H]1C(=O)N[C@H](C#N)C[C@@H]1CC(C)(C)NC1=O)C2(C)C] |
| 319 | 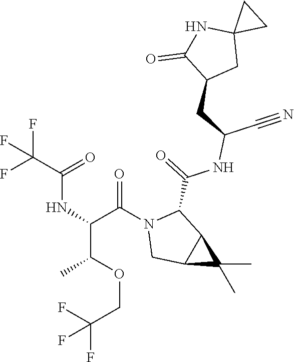 [SMILES: C[C@@H](OCC(F)(F)F)[C@H](NC(=O)C(F)(F)F)C(=O)N1C[C@H]2[C@@H]([C@H]1C(=O)N[C@H](C#N)C[C@@H]1CC3(CC3)NC1=O)C2(C)C] |
| 320 | 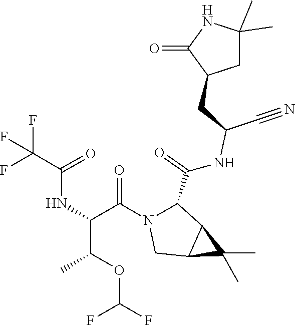 [SMILES: C[C@@H](OC(F)F)[C@H](NC(=O)C(F)(F)F)C(=O)N1C[C@H]2[C@@H]([C@H]1C(=O)N[C@H](C#N)C[C@@H]1CC(C)(C)NC1=O)C2(C)C] |
| 321 | 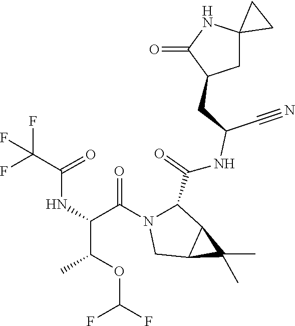 [SMILES: C[C@@H](OC(F)F)[C@H](NC(=O)C(F)(F)F)C(=O)N1C[C@H]2[C@@H]([C@H]1C(=O)N[C@H](C#N)C[C@@H]1CC3(CC3)NC1=O)C2(C)C] |
| 322 | 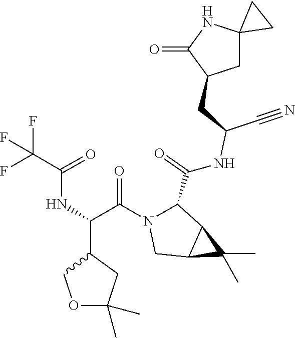 [SMILES: CC1(C)CC([C@H](NC(=O)C(F)(F)F)C(=O)N2C[C@H]3[C@@H]([C@H]2C(=O)N[C@H](C#N)C[C@@H]2CC4(CC4)NC2=O)C3(C)C)CO1] |
| 323 | 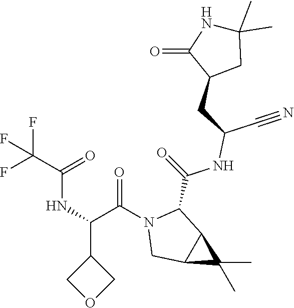 [SMILES: CC1(C)C[C@@H](C[C@@H](C#N)NC(=O)[C@@H]2[C@@H]3[C@H](CN2C(=O)[C@@H](NC(=O)C(F)(F)F)C2COC2)C3(C)C)C(=O)N1] |
| 324 | 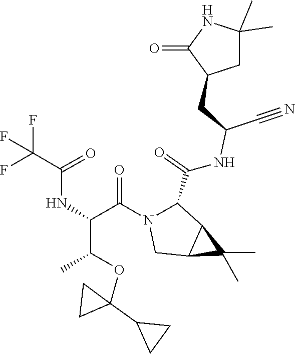 [SMILES: C[C@@H](OC1(C2CC2)CC1)[C@H](NC(=O)C(F)(F)F)C(=O)N1C[C@H]2[C@@H]([C@H]1C(=O)N[C@H](C#N)C[C@@H]1CC(C)(C)NC1=O)C2(C)C] |
| 325 | 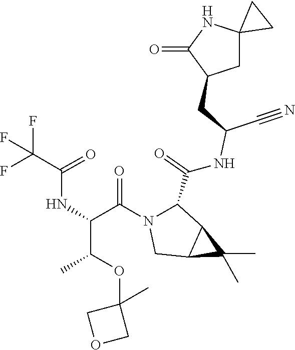 [SMILES: C[C@@H](OC1(C)COC1)[C@H](NC(=O)C(F)(F)F)C(=O)N1C[C@H]2[C@@H]([C@H]1C(=O)N[C@H](C#N)C[C@@H]1CC3(CC3)NC1=O)C2(C)C] |
| 326 | 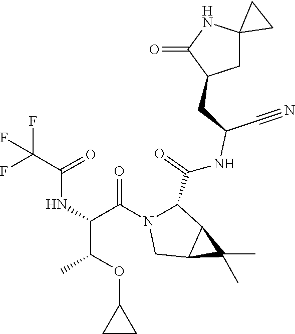 [SMILES: C[C@@H](OC1CC1)[C@H](NC(=O)C(F)(F)F)C(=O)N1C[C@H]2[C@@H]([C@H]1C(=O)N[C@H](C#N)C[C@@H]1CC3(CC3)NC1=O)C2(C)C] |
| 327 | 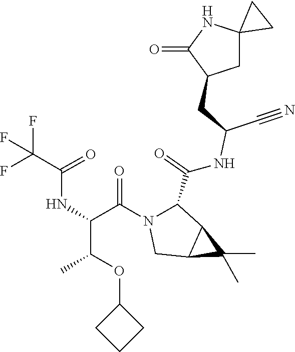 [SMILES: C[C@@H](OC1CCC1)[C@H](NC(=O)C(F)(F)F)C(=O)N1C[C@H]2[C@@H]([C@H]1C(=O)N[C@H](C#N)C[C@@H]1CC3(CC3)NC1=O)C2(C)C] |
| 328 | 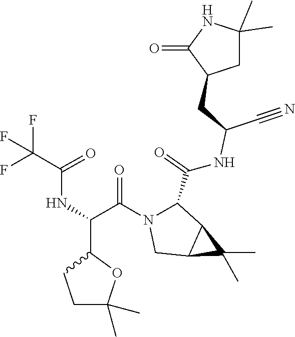 [SMILES: CC1(C)C[C@@H](C[C@@H](C#N)NC(=O)[C@@H]2[C@@H]3[C@H](CN2C(=O)[C@@H](NC(=O)C(F)(F)F)C2CCC(C)(C)O2)C3(C)C)C(=O)N1] |
| 329 | 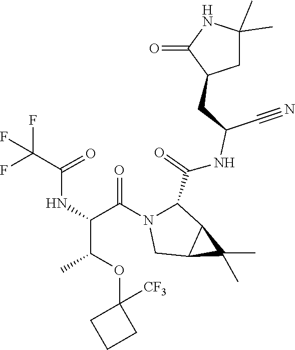 [SMILES: C[C@@H](OC1(C(F)(F)F)CCC1)[C@H](NC(=O)C(F)(F)F)C(=O)N1C[C@H]2[C@@H]([C@H]1C(=O)N[C@H](C#N)C[C@@H]1CC(C)(C)NC1=O)C2(C)C] |
| 330 | 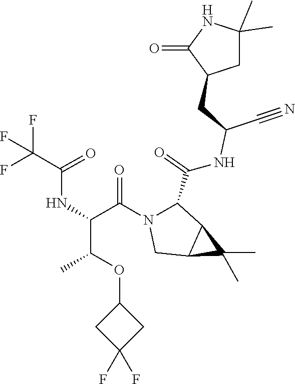 [SMILES: C[C@@H](OC1CC(F)(F)C1)[C@H](NC(=O)C(F)(F)F)C(=O)N1C[C@H]2[C@@H]([C@H]1C(=O)N[C@H](C#N)C[C@@H]1CC(C)(C)NC1=O)C2(C)C] |
| 331 | 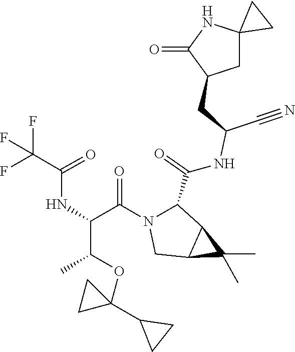 [SMILES: C[C@@H](OC1(C2CC2)CC1)[C@H](NC(=O)C(F)(F)F)C(=O)N1C[C@H]2[C@@H]([C@H]1C(=O)N[C@H](C#N)C[C@@H]1CC3(CC3)NC1=O)C2(C)C] |
| 332 | 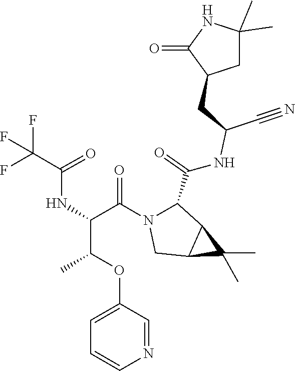 [SMILES: C[C@@H](Oc1cccnc1)[C@H](NC(=O)C(F)(F)F)C(=O)N1C[C@H]2[C@@H]([C@H]1C(=O)N[C@H](C#N)C[C@@H]1CC(C)(C)NC1=O)C2(C)C] |
| 333 | 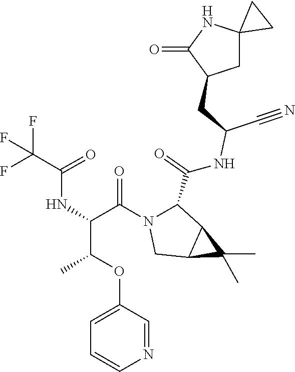 [SMILES: C[C@@H](Oc1cccnc1)[C@H](NC(=O)C(F)(F)F)C(=O)N1C[C@H]2[C@@H]([C@H]1C(=O)N[C@H](C#N)C[C@@H]1CC3(CC3)NC1=O)C2(C)C] |
| 334 | 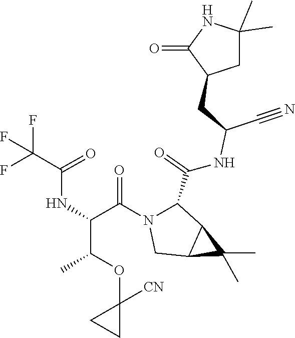 [SMILES: C[C@@H](OC1(C#N)CC1)[C@H](NC(=O)C(F)(F)F)C(=O)N1C[C@H]2[C@@H]([C@H]1C(=O)N[C@H](C#N)C[C@@H]1CC(C)(C)NC1=O)C2(C)C] |
| 335 | 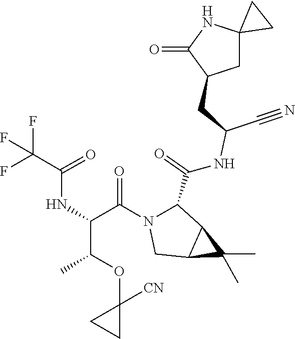 [SMILES: C[C@@H](OC1(C#N)CC1)[C@H](NC(=O)C(F)(F)F)C(=O)N1C[C@H]2[C@@H]([C@H]1C(=O)N[C@H](C#N)C[C@@H]1CC3(CC3)NC1=O)C2(C)C] |
| 336 | 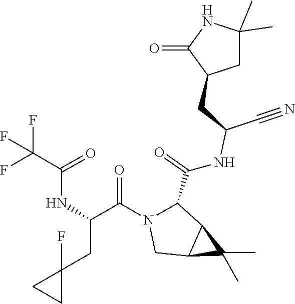 [SMILES: CC1(C)C[C@@H](C[C@@H](C#N)NC(=O)[C@@H]2[C@@H]3[C@H](CN2C(=O)[C@H](CC2(F)CC2)NC(=O)C(F)(F)F)C3(C)C)C(=O)N1] |
| 337 | 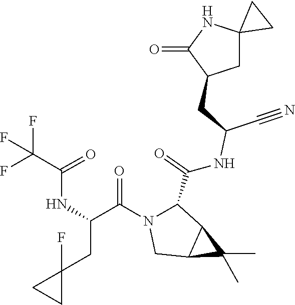 [SMILES: CC1(C)[C@@H]2[C@@H](C(=O)N[C@H](C#N)C[C@@H]3CC4(CC4)NC3=O)N(C(=O)[C@H](CC3(F)CC3)NC(=O)C(F)(F)F)C[C@@H]21] |
| 338 | 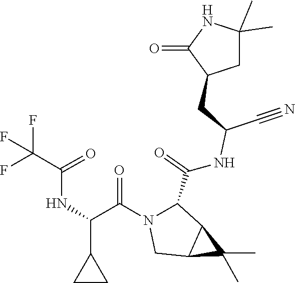 [SMILES: CC1(C)C[C@@H](C[C@@H](C#N)NC(=O)[C@@H]2[C@@H]3[C@H](CN2C(=O)[C@@H](NC(=O)C(F)(F)F)C2CC2)C3(C)C)C(=O)N1] |
| 339 | 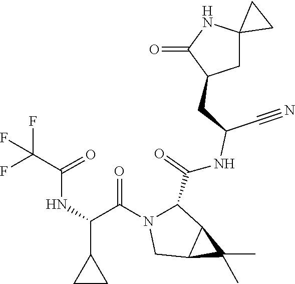 [SMILES: CC1(C)[C@@H]2[C@@H](C(=O)N[C@H](C#N)C[C@@H]3CC4(CC4)NC3=O)N(C(=O)[C@@H](NC(=O)C(F)(F)F)C3CC3)C[C@@H]21] |
| 340 | 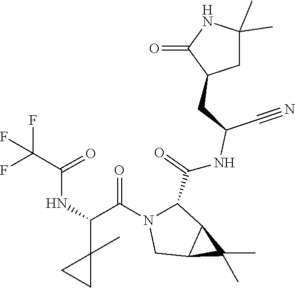 [SMILES: CC1(C)C[C@@H](C[C@@H](C#N)NC(=O)[C@@H]2[C@@H]3[C@H](CN2C(=O)[C@@H](NC(=O)C(F)(F)F)C2(C)CC2)C3(C)C)C(=O)N1] |
| 341 | 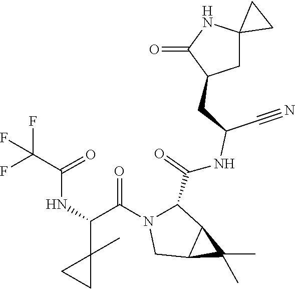 [SMILES: CC1([C@H](NC(=O)C(F)(F)F)C(=O)N2C[C@H]3[C@@H]([C@H]2C(=O)N[C@H](C#N)C[C@@H]2CC4(CC4)NC2=O)C3(C)C)CC1] |
| 342 | 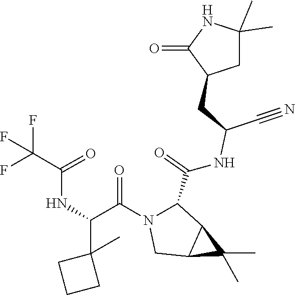 [SMILES: CC1(C)C[C@@H](C[C@@H](C#N)NC(=O)[C@@H]2[C@@H]3[C@H](CN2C(=O)[C@@H](NC(=O)C(F)(F)F)C2(C)CCC2)C3(C)C)C(=O)N1] |
| 343 | 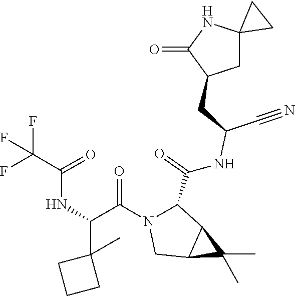 [SMILES: CC1([C@H](NC(=O)C(F)(F)F)C(=O)N2C[C@H]3[C@@H]([C@H]2C(=O)N[C@H](C#N)C[C@@H]2CC4(CC4)NC2=O)C3(C)C)CCC1] |
| 344 | 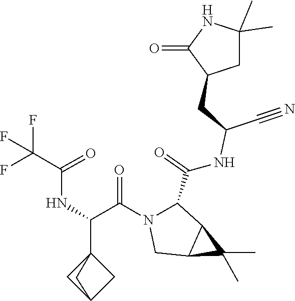 [SMILES: CC1(C)C[C@@H](C[C@@H](C#N)NC(=O)[C@@H]2[C@@H]3[C@H](CN2C(=O)[C@@H](NC(=O)C(F)(F)F)C24CC(C2)C4)C3(C)C)C(=O)N1] |
| 345 | 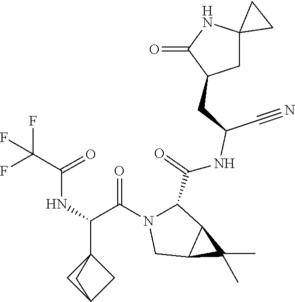 [SMILES: CC1(C)[C@@H]2[C@@H](C(=O)N[C@H](C#N)C[C@@H]3CC4(CC4)NC3=O)N(C(=O)[C@@H](NC(=O)C(F)(F)F)C34CC(C3)C4)C[C@@H]21] |
| 346 | 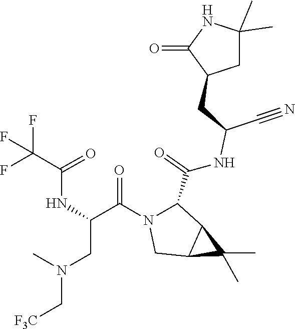 [SMILES: CN(C[C@H](NC(=O)C(F)(F)F)C(=O)N1C[C@H]2[C@@H]([C@H]1C(=O)N[C@H](C#N)C[C@@H]1CC(C)(C)NC1=O)C2(C)C)CC(F)(F)F] |
| 347 | 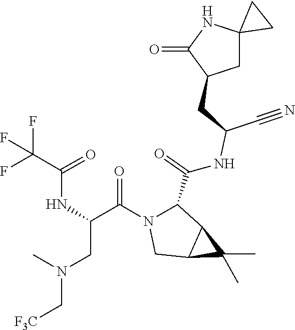 [SMILES: CN(C[C@H](NC(=O)C(F)(F)F)C(=O)N1C[C@H]2[C@@H]([C@H]1C(=O)N[C@H](C#N)C[C@@H]1CC3(CC3)NC1=O)C2(C)C)CC(F)(F)F] |
| 348 | 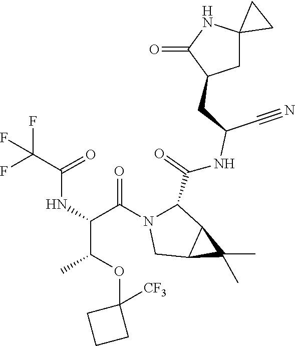 [SMILES: C[C@@H](OC1(C(F)(F)F)CCC1)[C@H](NC(=O)C(F)(F)F)C(=O)N1C[C@H]2[C@@H]([C@H]1C(=O)N[C@H](C#N)C[C@@H]1CC3(CC3)NC1=O)C2(C)C] |
| 349 | 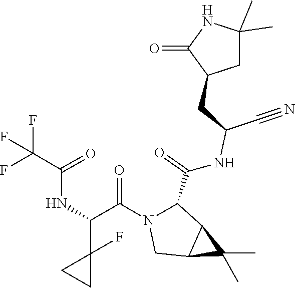 [SMILES: CC1(C)C[C@@H](C[C@@H](C#N)NC(=O)[C@@H]2[C@@H]3[C@H](CN2C(=O)[C@@H](NC(=O)C(F)(F)F)C2(F)CC2)C3(C)C)C(=O)N1] |
| 350 | 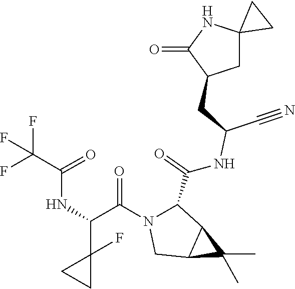 [SMILES: CC1(C)[C@@H]2[C@@H](C(=O)N[C@H](C#N)C[C@@H]3CC4(CC4)NC3=O)N(C(=O)[C@@H](NC(=O)C(F)(F)F)C3(F)CC3)C[C@@H]21] |
| 351 | 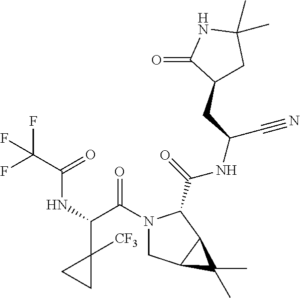 [SMILES: CC1(C)C[C@@H](C[C@@H](C#N)NC(=O)[C@@H]2[C@@H]3[C@H](CN2C(=O)[C@@H](NC(=O)C(F)(F)F)C2(C(F)(F)F)CC2)C3(C)C)C(=O)N1] |
| 352 | 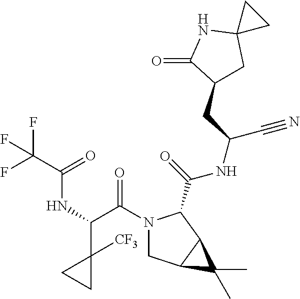 [SMILES: CC1(C)[C@@H]2[C@@H](C(=O)N[C@H](C#N)C[C@@H]3CC4(CC4)NC3=O)N(C(=O)[C@@H](NC(=O)C(F)(F)F)C3(C(F)(F)F)CC3)C[C@@H]21] |
| 353 | 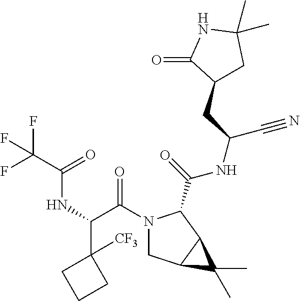 [SMILES: CC1(C)C[C@@H](C[C@@H](C#N)NC(=O)[C@@H]2[C@@H]3[C@H](CN2C(=O)[C@@H](NC(=O)C(F)(F)F)C2(C(F)(F)F)CCC2)C3(C)C)C(=O)N1] |
| 354 | 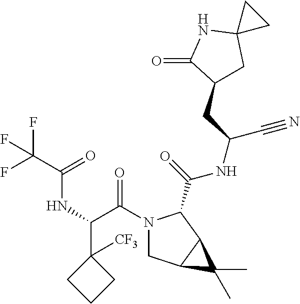 [SMILES: CC1(C)[C@@H]2[C@@H](C(=O)N[C@H](C#N)C[C@@H]3CC4(CC4)NC3=O)N(C(=O)[C@@H](NC(=O)C(F)(F)F)C3(C(F)(F)F)CCC3)C[C@@H]21] |
| 355 | 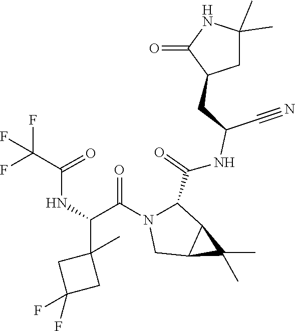 [SMILES: CC1(C)C[C@@H](C[C@@H](C#N)NC(=O)[C@@H]2[C@@H]3[C@H](CN2C(=O)[C@@H](NC(=O)C(F)(F)F)C2(C)CC(F)(F)C2)C3(C)C)C(=O)N1] |
| 356 | 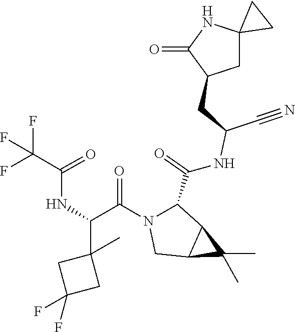 [SMILES: CC1([C@H](NC(=O)C(F)(F)F)C(=O)N2C[C@H]3[C@@H]([C@H]2C(=O)N[C@H](C#N)C[C@@H]2CC4(CC4)NC2=O)C3(C)C)CC(F)(F)C1] |
| 357 | 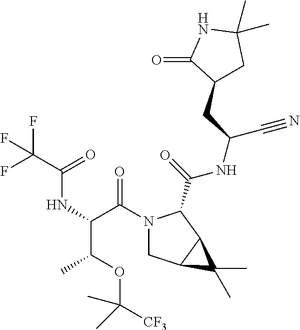 [SMILES: C[C@@H](OC(C)(C)C(F)(F)F)[C@H](NC(=O)C(F)(F)F)C(=O)N1C[C@H]2[C@@H]([C@H]1C(=O)N[C@H](C#N)C[C@@H]1CC(C)(C)NC1=O)C2(C)C] |
| 358 | 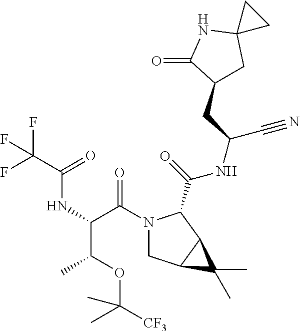 [SMILES: C[C@@H](OC(C)(C)C(F)(F)F)[C@H](NC(=O)C(F)(F)F)C(=O)N1C[C@H]2[C@@H]([C@H]1C(=O)N[C@H](C#N)C[C@@H]1CC3(CC3)NC1=O)C2(C)C] |
| 359 | 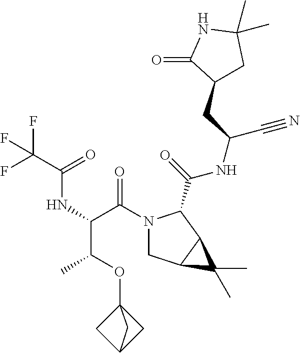 [SMILES: C[C@@H](OC12CC(C1)C2)[C@H](NC(=O)C(F)(F)F)C(=O)N1C[C@H]2[C@@H]([C@H]1C(=O)N[C@H](C#N)C[C@@H]1CC(C)(C)NC1=O)C2(C)C] |
| 360 | 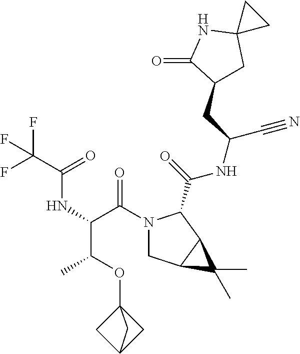 [SMILES: C[C@@H](OC12CC(C1)C2)[C@H](NC(=O)C(F)(F)F)C(=O)N1C[C@H]2[C@@H]([C@H]1C(=O)N[C@H](C#N)C[C@@H]1CC3(CC3)NC1=O)C2(C)C] |
| 361 | 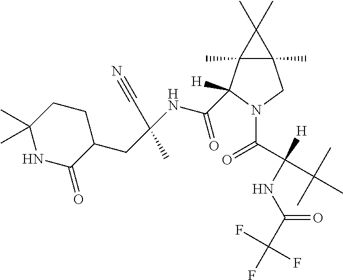 [SMILES: CC1(C)CCC(C[C@@](C)(C#N)NC(=O)[C@H]2N(C(=O)[C@@H](NC(=O)C(F)(F)F)C(C)(C)C)C[C@]3(C)C(C)(C)[C@]23C)C(=O)N1] |
| 362 | 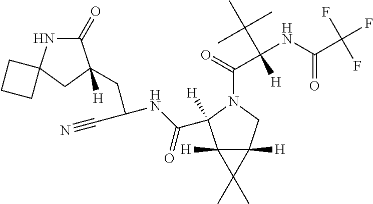 [SMILES: CC(C)(C)[C@H](NC(=O)C(F)(F)F)C(=O)N1C[C@H]2[C@@H]([C@H]1C(=O)NC(C#N)C[C@@H]1CC3(CCC3)NC1=O)C2(C)C] |
| 372 | 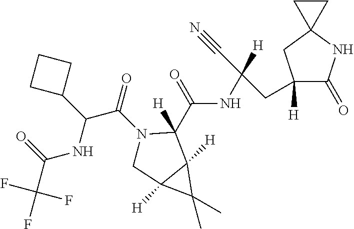 [SMILES: CC1(C)[C@@H]2[C@@H](C(=O)N[C@H](C#N)C[C@@H]3CC4(CC4)NC3=O)N(C(=O)C(NC(=O)C(F)(F)F)C3CCC3)C[C@@H]21] |
| 373 | 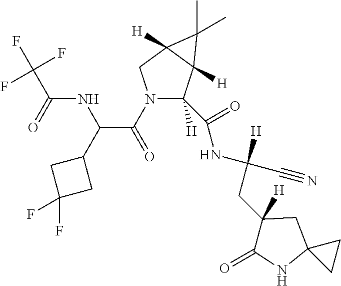 [SMILES: CC1(C)[C@@H]2[C@@H](C(=O)N[C@H](C#N)C[C@@H]3CC4(CC4)NC3=O)N(C(=O)C(NC(=O)C(F)(F)F)C3CC(F)(F)C3)C[C@@H]21] |
| 375 | 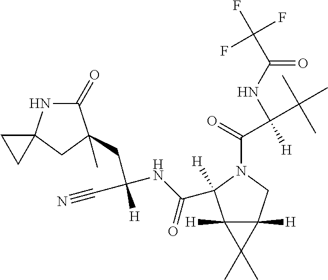 [SMILES: CC(C)(C)[C@H](NC(=O)C(F)(F)F)C(=O)N1C[C@H]2[C@@H]([C@H]1C(=O)N[C@H](C#N)C[C@@]1(C)CC3(CC3)NC1=O)C2(C)C] |
| 376 | 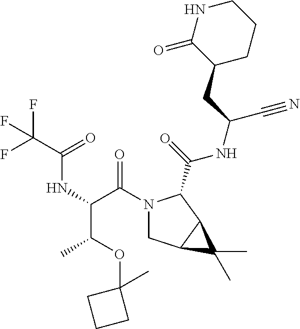 [SMILES: C[C@@H](OC1(C)CCC1)[C@H](NC(=O)C(F)(F)F)C(=O)N1C[C@H]2[C@@H]([C@H]1C(=O)N[C@H](C#N)C[C@@H]1CCCNC1=O)C2(C)C] |
| 377 | 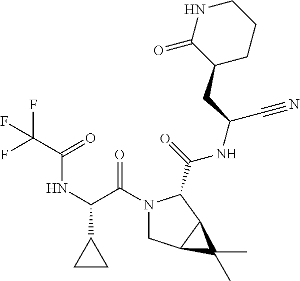 [SMILES: CC1(C)[C@@H]2[C@@H](C(=O)N[C@H](C#N)C[C@@H]3CCCNC3=O)N(C(=O)[C@@H](NC(=O)C(F)(F)F)C3CC3)C[C@@H]21] |
| 378 | 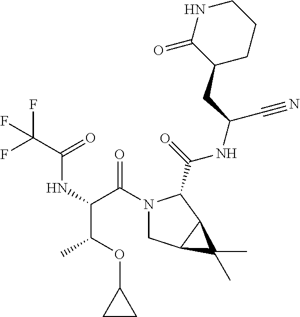 [SMILES: C[C@@H](OC1CC1)[C@H](NC(=O)C(F)(F)F)C(=O)N1C[C@H]2[C@@H]([C@H]1C(=O)N[C@H](C#N)C[C@@H]1CCCNC1=O)C2(C)C] |
| 425 | 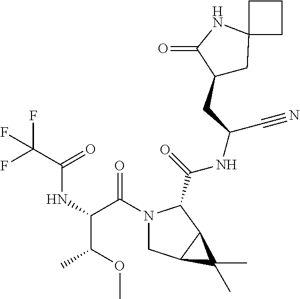 [SMILES: CO[C@H](C)[C@H](NC(=O)C(F)(F)F)C(=O)N1C[C@H]2[C@@H]([C@H]1C(=O)N[C@H](C#N)C[C@@H]1CC3(CCC3)NC1=O)C2(C)C] |
| 430 | 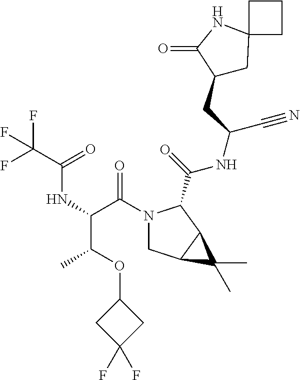 [SMILES: C[C@@H](OC1CC(F)(F)C1)[C@H](NC(=O)C(F)(F)F)C(=O)N1C[C@H]2[C@@H]([C@H]1C(=O)N[C@H](C#N)C[C@@H]1CC3(CCC3)NC1=O)C2(C)C] |
| 431 | 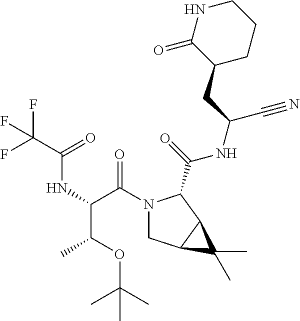 [SMILES: C[C@@H](OC(C)(C)C)[C@H](NC(=O)C(F)(F)F)C(=O)N1C[C@H]2[C@@H]([C@H]1C(=O)N[C@H](C#N)C[C@@H]1CCCNC1=O)C2(C)C] |
| 433 | 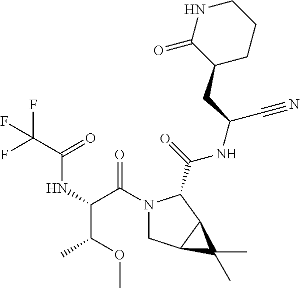 [SMILES: CO[C@H](C)[C@H](NC(=O)C(F)(F)F)C(=O)N1C[C@H]2[C@@H]([C@H]1C(=O)N[C@H](C#N)C[C@@H]1CCCNC1=O)C2(C)C] |
| 434 | 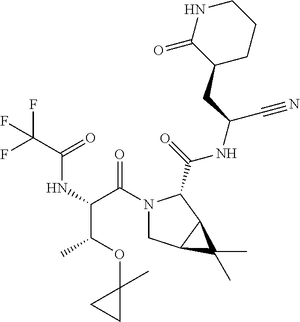 [SMILES: C[C@@H](OC1(C)CC1)[C@H](NC(=O)C(F)(F)F)C(=O)N1C[C@H]2[C@@H]([C@H]1C(=O)N[C@H](C#N)C[C@@H]1CCCNC1=O)C2(C)C] |
| 435 | 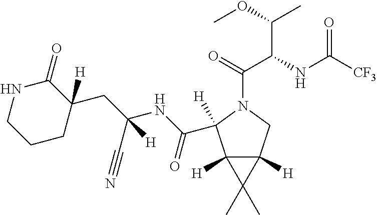 [SMILES: CO[C@H](C)[C@H](NC(=O)C(F)(F)F)C(=O)N1C[C@H]2[C@@H]([C@H]1C(=O)N[C@H](C#N)C[C@@H]1CCCNC1=O)C2(C)C] |
| 436 | 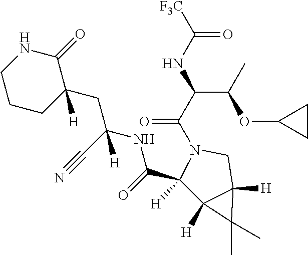 [SMILES: C[C@@H](OC1CC1)[C@H](NC(=O)C(F)(F)F)C(=O)N1C[C@H]2[C@@H]([C@H]1C(=O)N[C@H](C#N)C[C@@H]1CCCNC1=O)C2(C)C] |
| 437 | 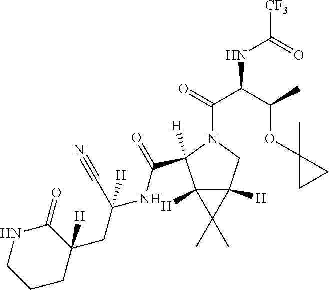 [SMILES: C[C@@H](OC1(C)CC1)[C@H](NC(=O)C(F)(F)F)C(=O)N1C[C@H]2[C@@H]([C@H]1C(=O)N[C@H](C#N)C[C@@H]1CCCNC1=O)C2(C)C] |
| 438 | 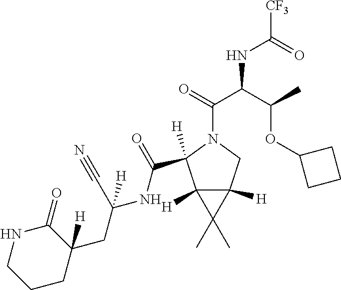 [SMILES: C[C@@H](OC1CCC1)[C@H](NC(=O)C(F)(F)F)C(=O)N1C[C@H]2[C@@H]([C@H]1C(=O)N[C@H](C#N)C[C@@H]1CCCNC1=O)C2(C)C] |
| 439 | 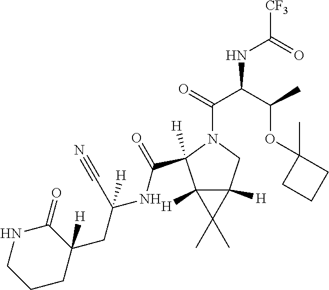 [SMILES: C[C@@H](OC1(C)CCC1)[C@H](NC(=O)C(F)(F)F)C(=O)N1C[C@H]2[C@@H]([C@H]1C(=O)N[C@H](C#N)C[C@@H]1CCCNC1=O)C2(C)C] |
| 440 | 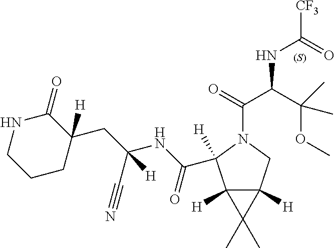 |
| 441 | 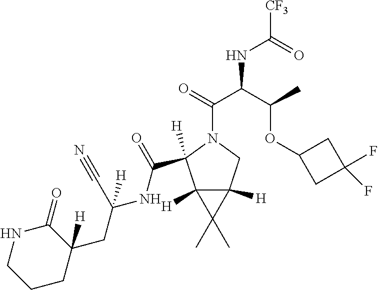 [SMILES: C[C@@H](OC1CC(F)(F)C1)[C@H](NC(=O)C(F)(F)F)C(=O)N1C[C@H]2[C@@H]([C@H]1C(=O)N[C@H](C#N)C[C@@H]1CCCNC1=O)C2(C)C] |
| 442 | 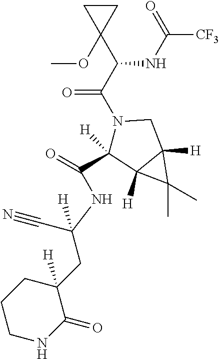 [SMILES: COC1([C@H](NC(=O)C(F)(F)F)C(=O)N2C[C@H]3[C@@H]([C@H]2C(=O)N[C@H](C#N)C[C@@H]2CCCNC2=O)C3(C)C)CC1] |
| 443 | 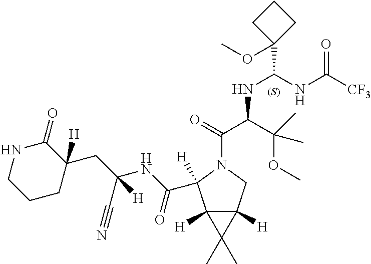 |
| 444 | 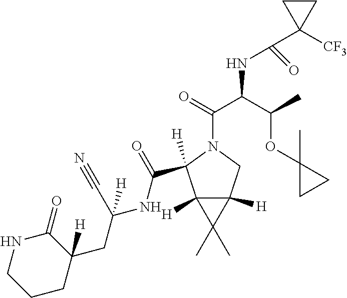 [SMILES: C[C@@H](OC1(C)CC1)[C@H](NC(=O)C1(C(F)(F)F)CC1)C(=O)N1C[C@H]2[C@@H]([C@H]1C(=O)N[C@H](C#N)C[C@@H]1CCCNC1=O)C2(C)C] |
| 445 | 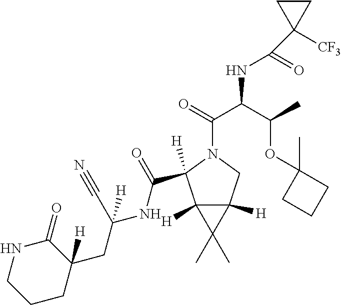 [SMILES: C[C@@H](OC1(C)CCC1)[C@H](NC(=O)C1(C(F)(F)F)CC1)C(=O)N1C[C@H]2[C@@H]([C@H]1C(=O)N[C@H](C#N)C[C@@H]1CCCNC1=O)C2(C)C] |
| 446 | 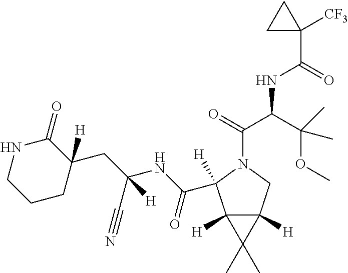 [SMILES: COC(C)(C)[C@H](NC(=O)C1(C(F)(F)F)CC1)C(=O)N1C[C@H]2[C@@H]([C@H]1C(=O)N[C@H](C#N)C[C@@H]1CCCNC1=O)C2(C)C] |
| 447 | 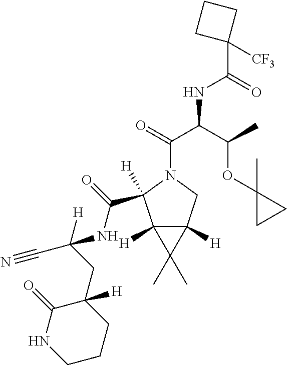 [SMILES: C[C@@H](OC1(C)CC1)[C@H](NC(=O)C1(C(F)(F)F)CCC1)C(=O)N1C[C@H]2[C@@H]([C@H]1C(=O)N[C@H](C#N)C[C@@H]1CCCNC1=O)C2(C)C] |
| 448 | 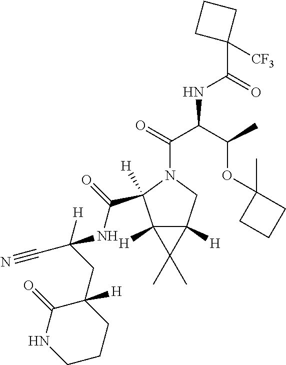 [SMILES: C[C@@H](OC1(C)CCC1)[C@H](NC(=O)C1(C(F)(F)F)CCC1)C(=O)N1C[C@H]2[C@@H]([C@H]1C(=O)N[C@H](C#N)C[C@@H]1CCCNC1=O)C2(C)C] |
| 449 | 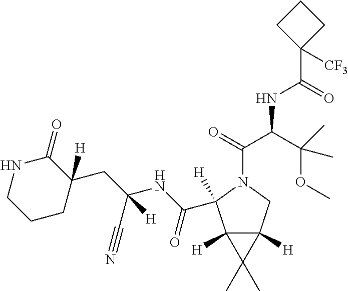 [SMILES: COC(C)(C)[C@H](NC(=O)C1(C(F)(F)F)CCC1)C(=O)N1C[C@H]2[C@@H]([C@H]1C(=O)N[C@H](C#N)C[C@@H]1CCCNC1=O)C2(C)C] |
| 450 | 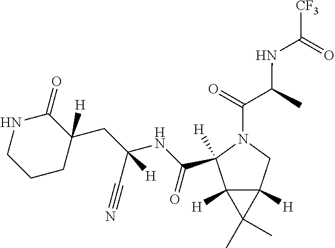 [SMILES: C[C@H](NC(=O)C(F)(F)F)C(=O)N1C[C@H]2[C@@H]([C@H]1C(=O)N[C@H](C#N)C[C@@H]1CCCNC1=O)C2(C)C] |
| 451 | 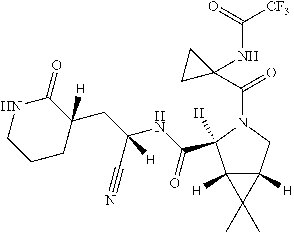 [SMILES: CC1(C)[C@@H]2[C@@H](C(=O)N[C@H](C#N)C[C@@H]3CCCNC3=O)N(C(=O)C3(NC(=O)C(F)(F)F)CC3)C[C@@H]21] |
| 452 | 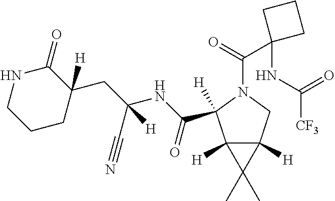 [SMILES: CC1(C)[C@@H]2[C@@H](C(=O)N[C@H](C#N)C[C@@H]3CCCNC3=O)N(C(=O)C3(NC(=O)C(F)(F)F)CCC3)C[C@@H]21] |
| 453 | 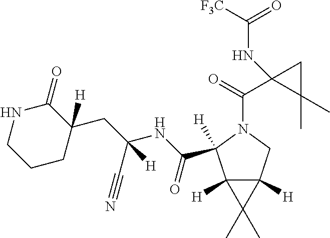 [SMILES: CC1(C)[C@@H]2[C@@H](C(=O)N[C@H](C#N)C[C@@H]3CCCNC3=O)N(C(=O)C3(NC(=O)C(F)(F)F)CC3(C)C)C[C@@H]21] |
| 454 | 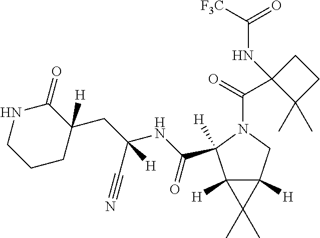 [SMILES: CC1(C)[C@@H]2[C@@H](C(=O)N[C@H](C#N)C[C@@H]3CCCNC3=O)N(C(=O)C3(NC(=O)C(F)(F)F)CCC3(C)C)C[C@@H]21] |
| 455 | 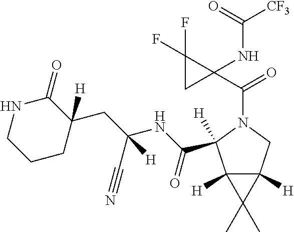 [SMILES: CC1(C)[C@@H]2[C@@H](C(=O)N[C@H](C#N)C[C@@H]3CCCNC3=O)N(C(=O)C3(NC(=O)C(F)(F)F)CC3(F)F)C[C@@H]21] |
| 456 | 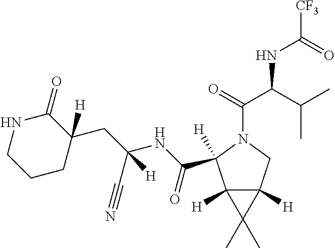 [SMILES: CC(C)[C@H](NC(=O)C(F)(F)F)C(=O)N1C[C@H]2[C@@H]([C@H]1C(=O)N[C@H](C#N)C[C@@H]1CCCNC1=O)C2(C)C] |
| 457 | 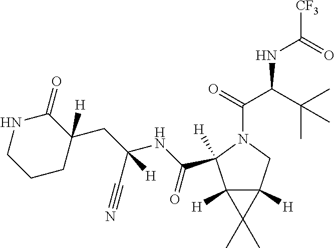 [SMILES: CC(C)(C)[C@H](NC(=O)C(F)(F)F)C(=O)N1C[C@H]2[C@@H]([C@H]1C(=O)N[C@H](C#N)C[C@@H]1CCCNC1=O)C2(C)C] |
| 458 | 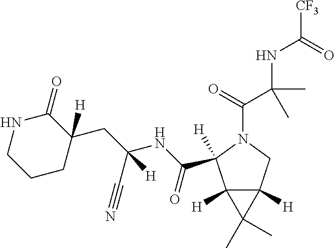 [SMILES: CC(C)(NC(=O)C(F)(F)F)C(=O)N1C[C@H]2[C@@H]([C@H]1C(=O)N[C@H](C#N)C[C@@H]1CCCNC1=O)C2(C)C] |
| 459 | 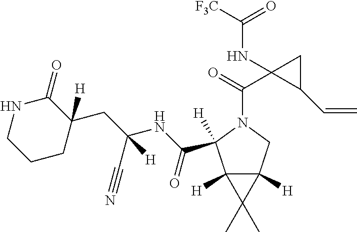 [SMILES: C=CC1CC1(NC(=O)C(F)(F)F)C(=O)N1C[C@H]2[C@@H]([C@H]1C(=O)N[C@H](C#N)C[C@@H]1CCCNC1=O)C2(C)C] |
| 460 | 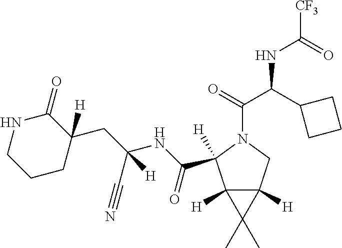 [SMILES: CC1(C)[C@@H]2[C@@H](C(=O)N[C@H](C#N)C[C@@H]3CCCNC3=O)N(C(=O)[C@@H](NC(=O)C(F)(F)F)C3CCC3)C[C@@H]21] |
| 461 | 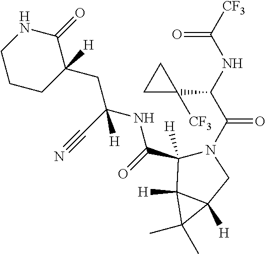 [SMILES: CC1(C)[C@@H]2[C@@H](C(=O)N[C@H](C#N)C[C@@H]3CCCNC3=O)N(C(=O)[C@@H](NC(=O)C(F)(F)F)C3(C(F)(F)F)CC3)C[C@@H]21] |
| 462 | 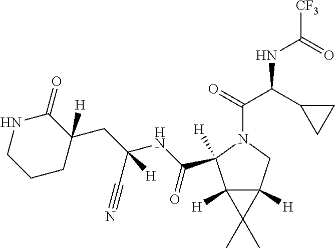 [SMILES: CC1(C)[C@@H]2[C@@H](C(=O)N[C@H](C#N)C[C@@H]3CCCNC3=O)N(C(=O)[C@@H](NC(=O)C(F)(F)F)C3CC3)C[C@@H]21] |
| 463 | 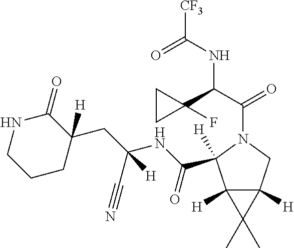 [SMILES: CC1(C)[C@@H]2[C@@H](C(=O)N[C@H](C#N)C[C@@H]3CCCNC3=O)N(C(=O)[C@H](NC(=O)C(F)(F)F)C3(F)CC3)C[C@@H]21] |
| 464 | 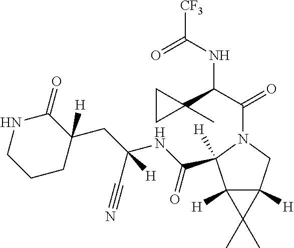 [SMILES: CC1([C@@H](NC(=O)C(F)(F)F)C(=O)N2C[C@H]3[C@@H]([C@H]2C(=O)N[C@H](C#N)C[C@@H]2CCCNC2=O)C3(C)C)CC1] |
| 465 | 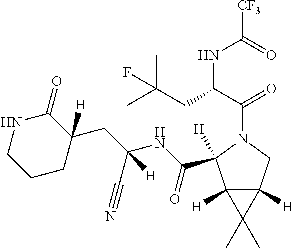 [SMILES: CC(C)(F)C[C@H](NC(=O)C(F)(F)F)C(=O)N1C[C@H]2[C@@H]([C@H]1C(=O)N[C@H](C#N)C[C@@H]1CCCNC1=O)C2(C)C] |
| 466 | 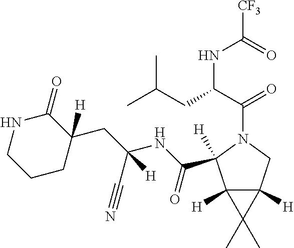 [SMILES: CC(C)C[C@H](NC(=O)C(F)(F)F)C(=O)N1C[C@H]2[C@@H]([C@H]1C(=O)N[C@H](C#N)C[C@@H]1CCCNC1=O)C2(C)C] |
| 467 | 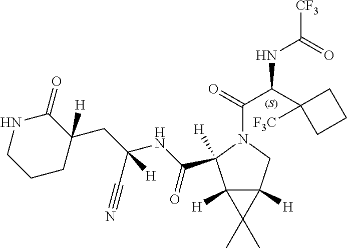 |
| 468 | 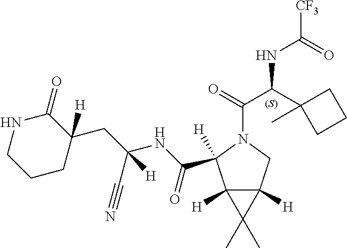 |
| 469 | 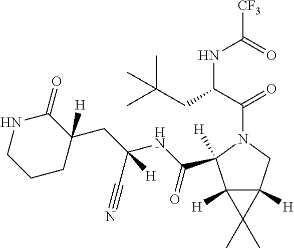 [SMILES: CC(C)(C)C[C@H](NC(=O)C(F)(F)F)C(=O)N1C[C@H]2[C@@H]([C@H]1C(=O)N[C@H](C#N)C[C@@H]1CCCNC1=O)C2(C)C] |
| 470 | 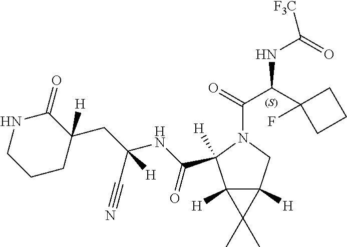 |
| 471 | 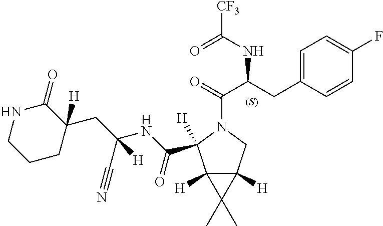 |
| 472 | 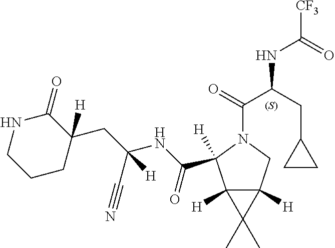 |
| 473 | 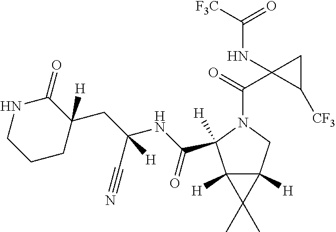 [SMILES: CC1(C)[C@@H]2[C@@H](C(=O)N[C@H](C#N)C[C@@H]3CCCNC3=O)N(C(=O)C3(NC(=O)C(F)(F)F)CC3C(F)(F)F)C[C@@H]21] |
| 474 | 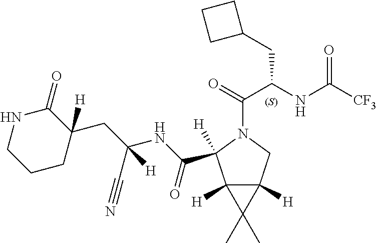 |
| 475 | 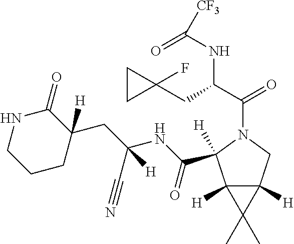 [SMILES: CC1(C)[C@@H]2[C@@H](C(=O)N[C@H](C#N)C[C@@H]3CCCNC3=O)N(C(=O)[C@H](CC3(F)CC3)NC(=O)C(F)(F)F)C[C@@H]21] |
| 476 | 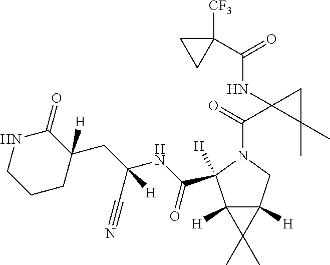 [SMILES: CC1(C)[C@@H]2[C@@H](C(=O)N[C@H](C#N)C[C@@H]3CCCNC3=O)N(C(=O)C3(NC(=O)C4(C(F)(F)F)CC4)CC3(C)C)C[C@@H]21] |
| 477 | 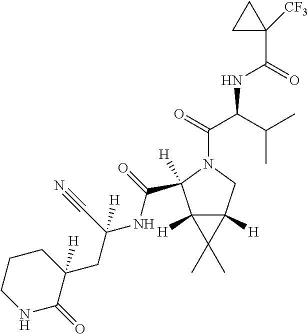 [SMILES: CC(C)[C@H](NC(=O)C1(C(F)(F)F)CC1)C(=O)N1C[C@H]2[C@@H]([C@H]1C(=O)N[C@H](C#N)C[C@@H]1CCCNC1=O)C2(C)C] |
| 478 | 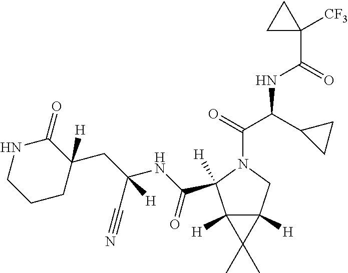 [SMILES: CC1(C)[C@@H]2[C@@H](C(=O)N[C@H](C#N)C[C@@H]3CCCNC3=O)N(C(=O)[C@@H](NC(=O)C3(C(F)(F)F)CC3)C3CC3)C[C@@H]21] |
| 479 | 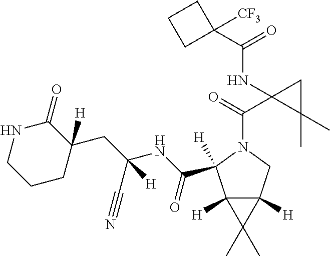 [SMILES: CC1(C)[C@@H]2[C@@H](C(=O)N[C@H](C#N)C[C@@H]3CCCNC3=O)N(C(=O)C3(NC(=O)C4(C(F)(F)F)CCC4)CC3(C)C)C[C@@H]21] |
| 480 | 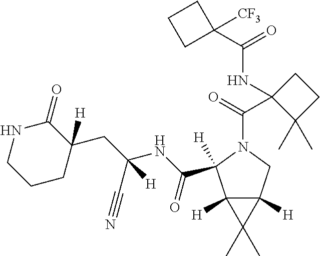 [SMILES: CC1(C)[C@@H]2[C@@H](C(=O)N[C@H](C#N)C[C@@H]3CCCNC3=O)N(C(=O)C3(NC(=O)C4(C(F)(F)F)CCC4)CCC3(C)C)C[C@@H]21] |
| 481 | 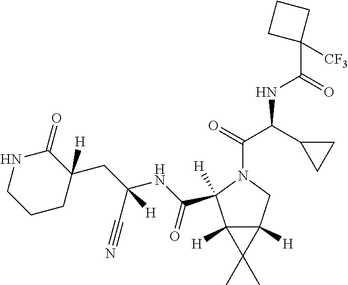 [SMILES: CC1(C)[C@@H]2[C@@H](C(=O)N[C@H](C#N)C[C@@H]3CCCNC3=O)N(C(=O)[C@@H](NC(=O)C3(C(F)(F)F)CCC3)C3CC3)C[C@@H]21] |
| 482 | 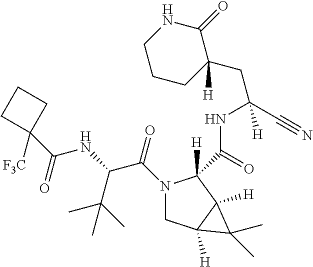 [SMILES: CC(C)(C)[C@H](NC(=O)C1(C(F)(F)F)CCC1)C(=O)N1C[C@H]2[C@@H]([C@H]1C(=O)N[C@H](C#N)C[C@@H]1CCCNC1=O)C2(C)C] |
| 483 | 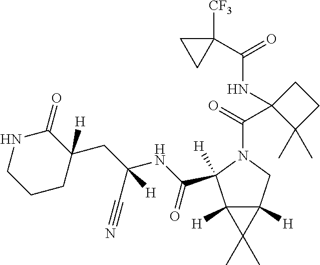 [SMILES: CC1(C)[C@@H]2[C@@H](C(=O)N[C@H](C#N)C[C@@H]3CCCNC3=O)N(C(=O)C3(NC(=O)C4(C(F)(F)F)CC4)CCC3(C)C)C[C@@H]21] |
| 484 | 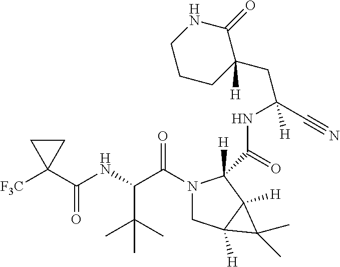 [SMILES: CC(C)(C)[C@H](NC(=O)C1(C(F)(F)F)CC1)C(=O)N1C[C@H]2[C@@H]([C@H]1C(=O)N[C@H](C#N)C[C@@H]1CCCNC1=O)C2(C)C] |
| 485 | 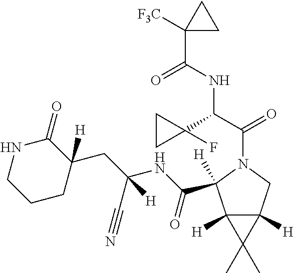 [SMILES: CC1(C)[C@@H]2[C@@H](C(=O)N[C@H](C#N)C[C@@H]3CCCNC3=O)N(C(=O)[C@@H](NC(=O)C3(C(F)(F)F)CC3)C3(F)CC3)C[C@@H]21] |
| 486 | 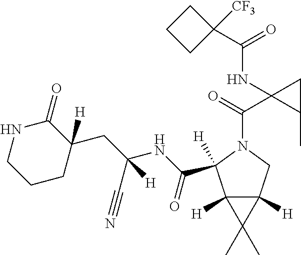 [SMILES: CC1CC1(NC(=O)C1(C(F)(F)F)CCC1)C(=O)N1C[C@H]2[C@@H]([C@H]1C(=O)N[C@H](C#N)C[C@@H]1CCCNC1=O)C2(C)C] |
| 487 | 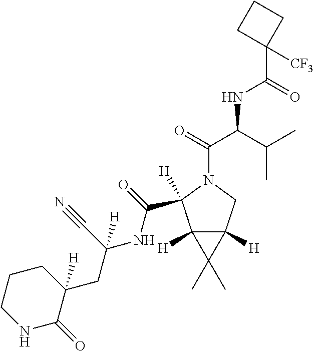 [SMILES: CC(C)[C@H](NC(=O)C1(C(F)(F)F)CCC1)C(=O)N1C[C@H]2[C@@H]([C@H]1C(=O)N[C@H](C#N)C[C@@H]1CCCNC1=O)C2(C)C] |
| 488 | 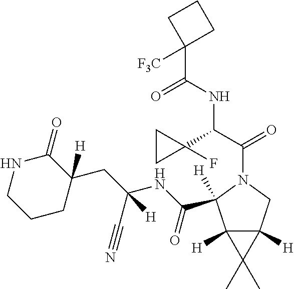 [SMILES: CC1(C)[C@@H]2[C@@H](C(=O)N[C@H](C#N)C[C@@H]3CCCNC3=O)N(C(=O)[C@@H](NC(=O)C3(C(F)(F)F)CCC3)C3(F)CC3)C[C@@H]21] |

In embodiments, disclosed herein is a protease inhibitory compound represented by Formula X:

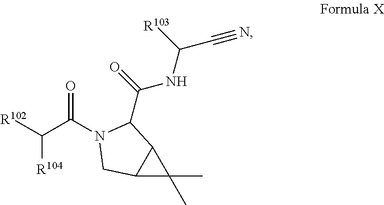

or a pharmaceutically acceptable salt, and/or a stereoisomer thereof, wherein: R¹⁰⁴ is selected from the group consisting of

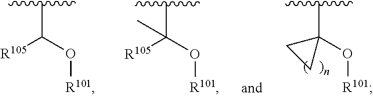

wherein R¹⁰⁵ is C₁-C₆alkyl, and n is 1, 2, or 3; R¹⁰¹ is selected from the group consisting of C₁-C₆alkyl, C₂-C₆alkenyl, C₃-C₆cycloalkyl, and C₃-C₆cycloalkenyl, wherein R¹⁰¹ may optionally be substituted by one, two, three, or four substituents each independently selected from R^{D}; R^{D} is selected from the group consisting of halogen, C₁-C₆alkyl, C₁-C₆alkoxy, C₃-C₆cycloalkyl, and cyano, wherein the alkyl may optionally be substituted by one or more halo; R¹⁰² is —NR^{m}(C═O)R^{m}; R^{m} is, for each occurrence, selected from the group consisting of H, C₁-6alkyl and C₃-C₆cycloalkyl, wherein the alkyl or cycloalkyl is optionally substituted by one, two or three halo or —CF₃; R¹⁰³ is

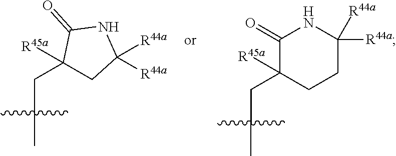

R^{44a} is each independently selected from the group consisting of hydrogen, —CH₃ and —CF₃, or two R^{44a} groups, together with the carbon to which they are attached, may be joined together to form a cyclopropyl, cyclobutyl, or oxetanyl; and R^{45a} is H or C₁-C₆alkyl.

In some embodiments, R¹⁰³ is

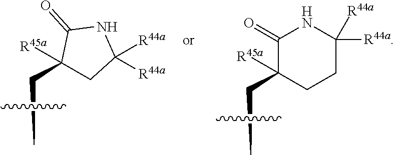

In another example, disclosed herein is a protease inhibitory compound represented by Formula X:

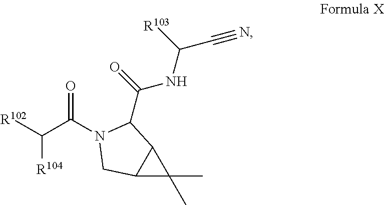

or a pharmaceutically acceptable salt, and/or a stereoisomer thereof, wherein: R¹⁰⁴ is selected from the group consisting of

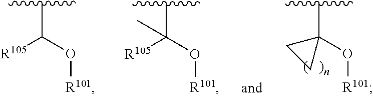

wherein R¹⁰⁵ is C₁-C₆alkyl, and n is 1, 2, or 3; R¹⁰¹ is selected from the group consisting of C₁-C₆alkyl, C₂-C₆alkenyl, C₃-C₆cycloalkyl, and C₃-C₆cycloalkenyl, wherein R¹⁰¹ may optionally be substituted by one, two, three, or four substituents each independently selected from R^{D}; R^{D} is selected from the group consisting of halogen, C₁-C₆alkyl, C₁-C₆alkoxy, C₃-C₆cycloalkyl, and cyano, wherein the alkyl may optionally be substituted by one or more halo; R¹⁰² is —NR^{m}(C═O)R^{m}; R^{m} is, for each occurrence, selected from the group consisting of H, C₁-alkyl and C₃-C₆cycloalkyl, wherein the alkyl or cycloalkyl is optionally substituted by one, two or three halo or —CF₃; R¹⁰³ is

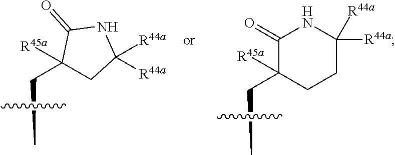

R^{44a} is each independently selected from the group consisting of hydrogen, —CH₃ and —CF₃, or two R^{44a} groups, together with the carbon to which they are attached, may be joined together to form a cyclopropyl, cyclobutyl, or oxetanyl; and R^{45a} is H or C₁-C₆alkyl.

In some embodiments, Formula X is represented by:

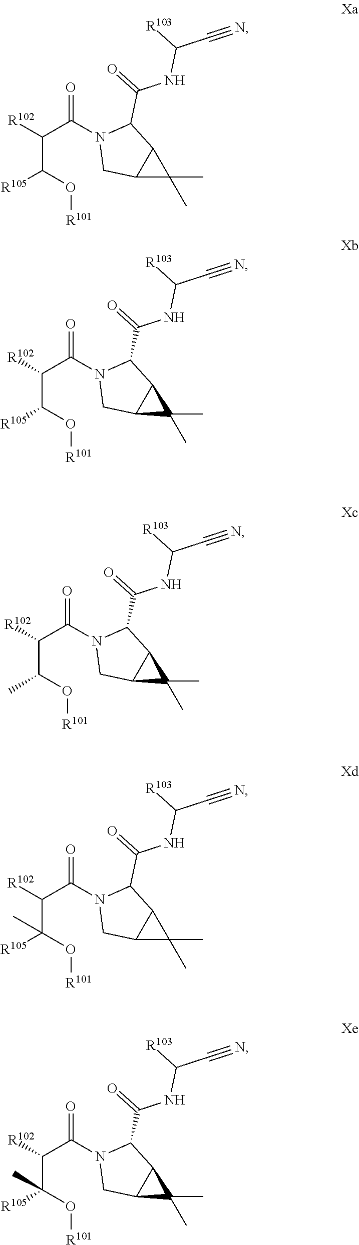

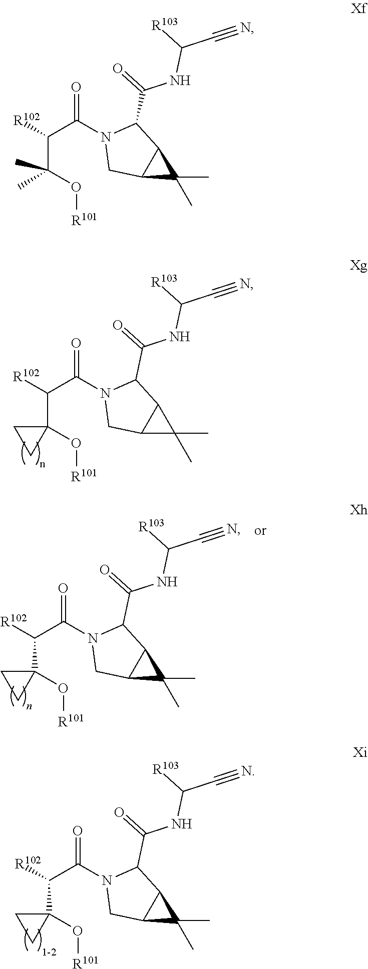

In some embodiments, R¹⁰⁴ is

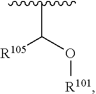

R¹⁰¹ is C₁-C₆alkyl or C₃-C₆cycloalkyl, and R¹⁰⁵ is C₁-C₆alkyl, wherein the R¹⁰¹ is optionally substituted by one or two substituents each independently selected from the group consisting of —F, —CF₃, —CH₃, —CN and cyclopropyl.

In some embodiments,

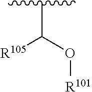

is selected from the group consisting of

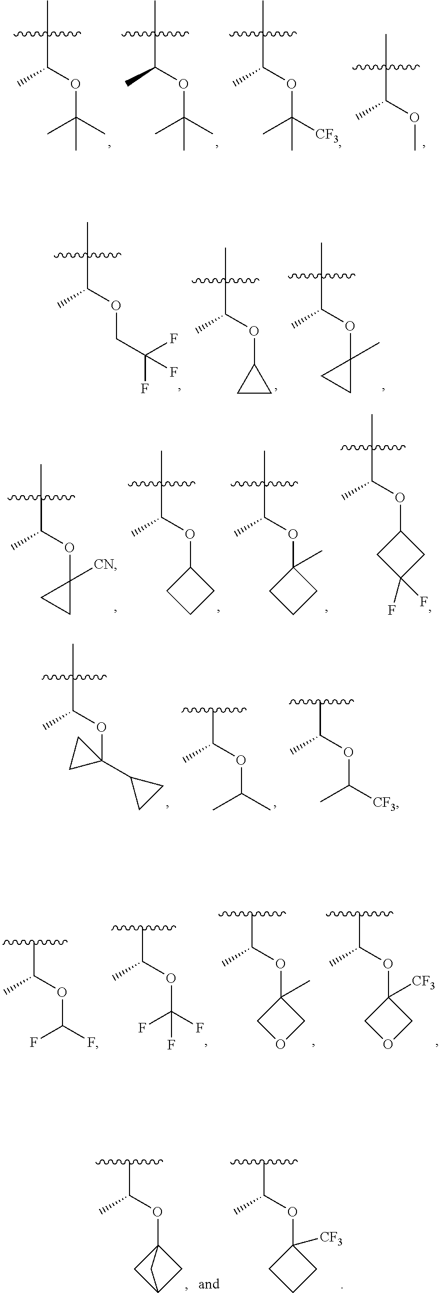

In some embodiments, the

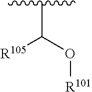

is selected from the group consisting of

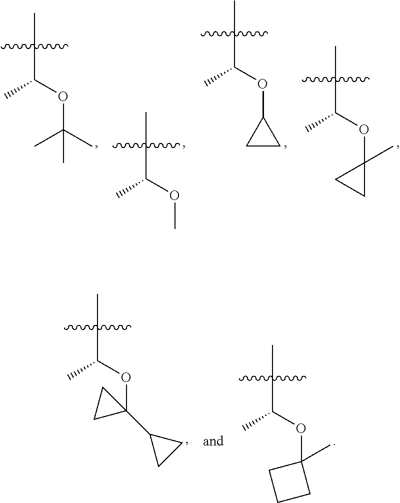

In some embodiments, R¹⁰⁴ is

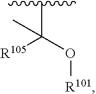

R¹⁰¹ is C₁-C₆alkyl or C₃-C₆cycloalkyl, and R¹⁰⁵ is C₁-C₆alkyl, wherein the R¹⁰¹ is optionally substituted by one or two substituents each independently selected from the group consisting of —F, —CF₃, —CH₃, —CN and cyclopropyl.

In some embodiments, the

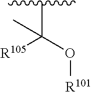

is selected from the group consisting of

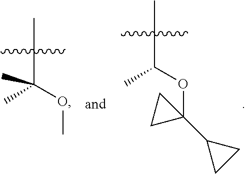

In some embodiments, R¹⁰¹ is selected from the group consisting of:

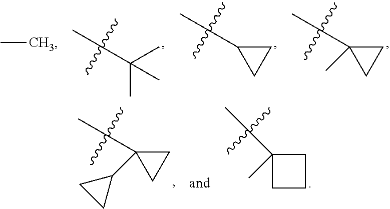

In some embodiments, R¹⁰¹ is selected from the group consisting of:

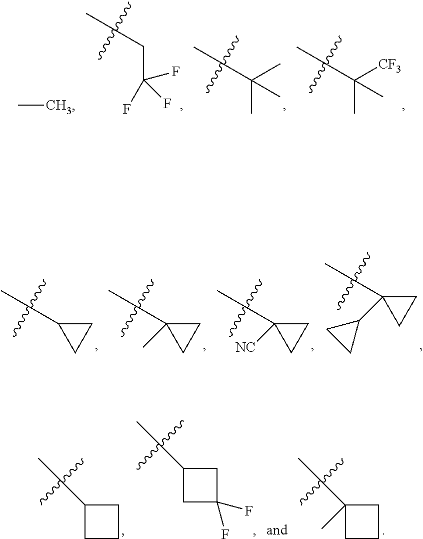

In some embodiments, R¹⁰⁴ is

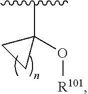

R¹⁰¹ is C₁-C₆alkyl, and n is 1 or 2.

In some embodiments,

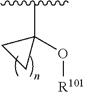

is selected from

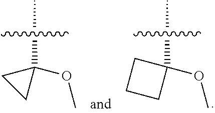

In some embodiments, n is 1. In some embodiments, n is 2. In some embodiments, n is 3.

In some embodiments, R¹⁰⁵ is —CH₃.

In some embodiments, R¹⁰² is —NH(C═O)CF₃.

In some embodiments, R¹⁰³ is

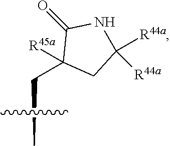

R^{44a} is each independently —CH₃, and R^{45a} is H.

In some embodiments, R¹⁰³ is

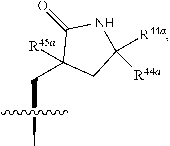

two R⁴⁴ groups, together with the carbon to which they are attached, are joined together to form a cyclopropyl, and R^{45a} is H.

In some embodiments, R¹⁰³ is

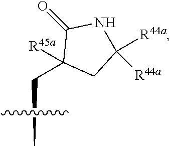

two R⁴⁴ groups, together with the carbon to which they are attached, are joined together to form a cyclobutyl, and R^{45a} is H.

In some embodiments, R¹⁰³ is

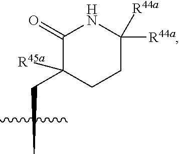

R^{44a} is each independently —CH₃, and R^{45a} is H.

In some embodiments, R¹⁰³ is

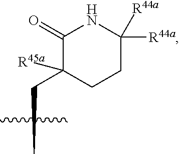

R^{44a} is H, and R^{45a} is H.

In some embodiments, R¹⁰³ is

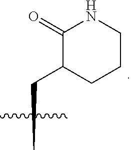

In some embodiments, R¹⁰³ is

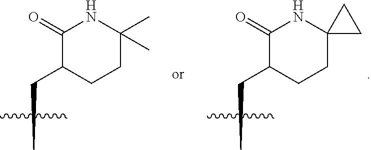

In some embodiments, R¹⁰³ is

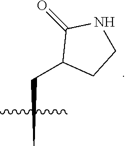

In some embodiments, R¹⁰³ is

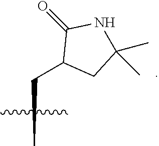

In some embodiments, R¹⁰³ is

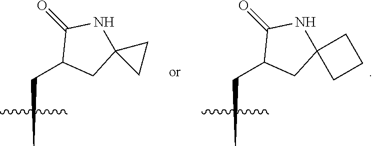

In some embodiments, R^{D} is selected from the group consisting of methoxy, halo, —CH₃, and cyano. In some embodiments, R^{D} is C₁-C₆alkyl, wherein the C₁-C₆alkyl is optionally substituted by one, two or three halos. In some embodiments, one of the carbons in the alkyl or alkenyl in the R¹⁰¹ may optionally be replaced by an oxygen.

In another aspect of this disclosure, provided herein is a protease inhibitory compound represented by Formula XI:

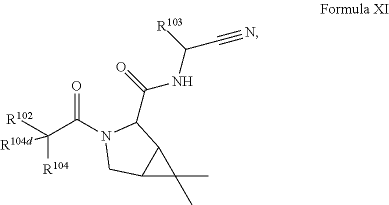

or a pharmaceutically acceptable salt, and/or a stereoisomer thereof, wherein: R¹⁰⁴ is selected from the group consisting of

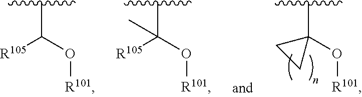

wherein R¹⁰⁵ is C₁-C₂alkyl, and n is 1, or 2; R^{104d} is H; or R^{104a} and R^{104d}, together with the carbon atom to which they are attached, may be joined to form a 4-6 membered heterocyclyl; R¹⁰¹ is selected from the group consisting of C₁-C₆alkyl, C₂-C₆alkenyl, C₃-C₆cycloalkyl, and C₃-C₆cycloalkenyl, wherein R¹⁰¹ may optionally be substituted by one, two, three, or four substituents each independently selected from R^{F}; or R¹⁰¹ and R¹⁰⁵ may be joined, together with the atoms to which they are attached, to form a 4-6 membered heterocyclyl, wherein the heterocyclyl may optionally be substituted by one, two, three, or four substituents each independently selected from the group consisting of halogen, and C₁-C₆alkyl; R^{F} is selected from the group consisting of halogen, C₁-C₂alkyl, C₁-C₆alkoxy, C₃-C₆cycloalkyl, and cyano, wherein the alkyl may optionally be substituted by one or more halo; R¹⁰² is selected from —NR^{m}(C═O)Rⁿ and

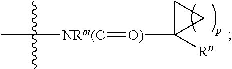

p is selected from 1 and 2; R^{m} is H; Rⁿ is selected from C₁₋₂alkyl and C₃-C₄cycloalkyl, wherein the alkyl or cycloalkyl is optionally substituted by one, two or three halo or —CF₃; R¹⁰³ is selected from

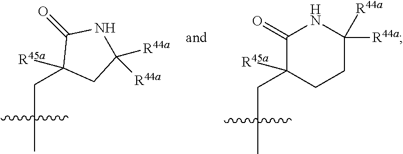

R^{44a} is each independently selected from the group consisting of hydrogen, —CH₃ and —CF₃, or two R^{44a} groups, together with the carbon to which they are attached, may be joined together to form a cyclopropyl, cyclobutyl, or oxetanyl; and R^{45a} is H, CH₃ or halo.

In some embodiments, R¹⁰³ is selected from

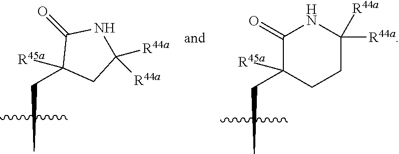

In some embodiments, R¹⁰¹ is selected from the group consisting of C₁-C₆alkyl, C₂-C₆alkenyl, C₃-C₆cycloalkyl, C₃-C₆cycloalkenyl, phenyl and pyridyl, wherein R¹⁰¹ may optionally be substituted by one, two, three, or four substituents each independently selected from R^{F}; wherein R^{F} is selected from the group consisting of halogen, C₁-C₂alkyl, C₁-C₆alkoxy, C₃-C₆cycloalkyl, and cyano, wherein the alkyl may optionally be substituted by one or more halo.

In some embodiments, Formula XI is represented by a formula selected from the group consisting of

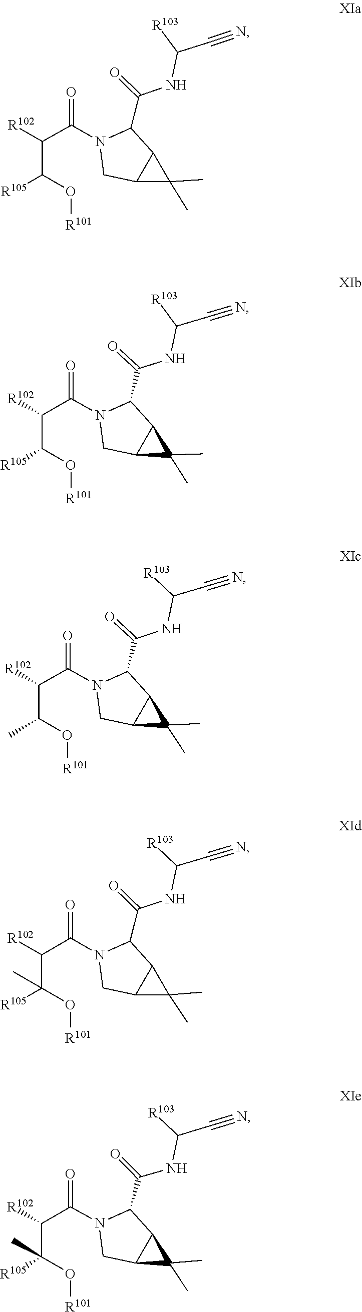

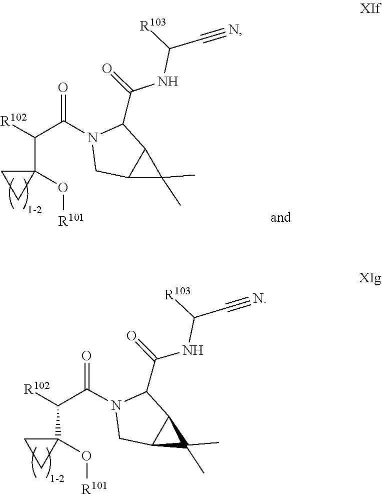

In some embodiments, R¹⁰⁴ is

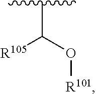

R¹⁰¹ is C₁-C₃alkyl or C₃-C₄cycloalkyl, and R¹⁰⁵ is C₁-C₂alkyl, wherein the R¹⁰¹ is optionally substituted by one or two substituents each independently selected from the group consisting of —F, —CF₃, —CH₃, —CN and cyclopropyl.

In some embodiments,

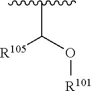

is selected from the Qroun consisting of

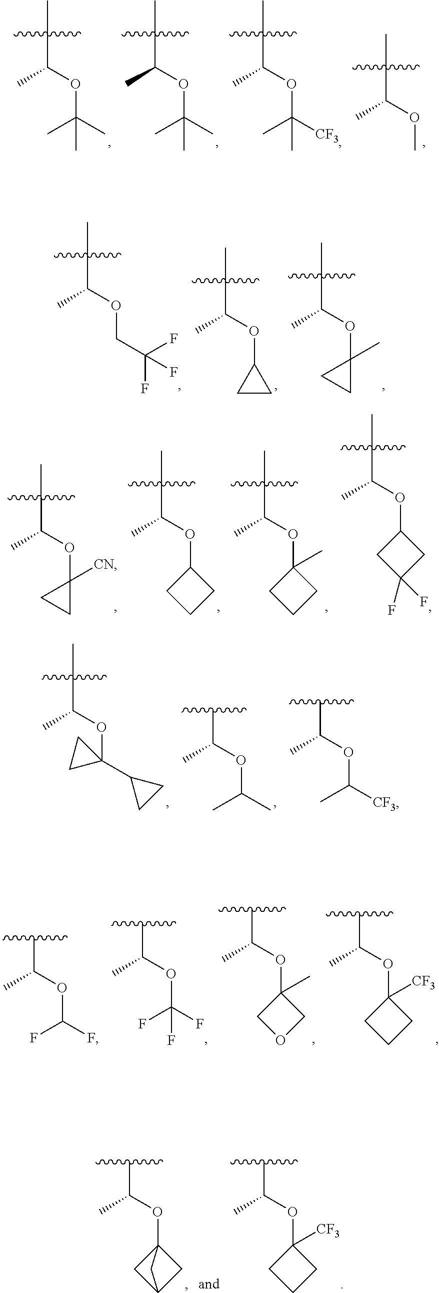

In some embodiments,

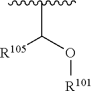

is selected from the group consisting of

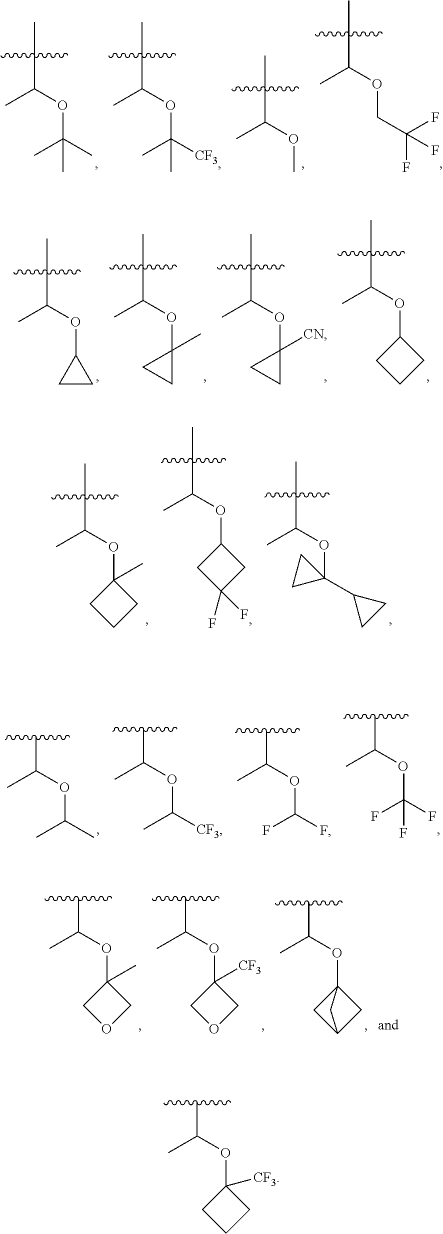

In some embodiments,

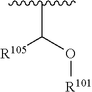

is selected from

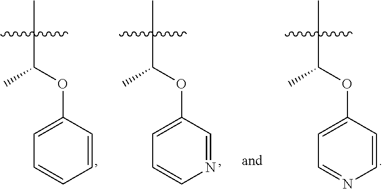

In some embodiments, R¹⁰⁴ is

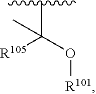

R¹⁰¹ is selected from the group consisting of C₁-C₆alkyl and C₃-C₆cycloalkyl, and R¹⁰⁵ is C₁-C₂alkyl, wherein the R¹⁰¹ is optionally substituted by one or two substituents each independently selected from the group consisting of —F, —CF₃, —CH₃, —CN and cyclopropyl.

In some embodiments, R¹⁰⁴ is

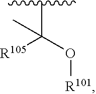

R¹⁰¹ is selected from the group consisting of C₁-C₆alkyl, C₃-C₆cycloalkyl, phenyl and pyridyl, and R¹⁰⁵ is C₁-C₂alkyl, wherein the R¹⁰¹ is optionally substituted by one or two substituents each independently selected from the group consisting of —F, —CF₃, —CH₃, —CN, cyclopropyl, phenyl and pyridyl.

In some embodiments,

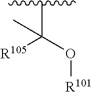

is selected from the group consisting of

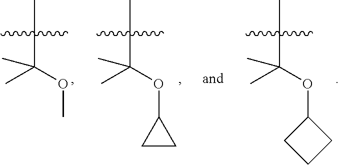

In some embodiments,

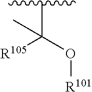

is

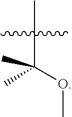

In some embodiments,

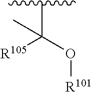

is

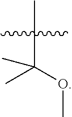 [SMILES: *C(C)(C)OC]

In some embodiments. R¹⁰¹ is selected from the group consisting of:

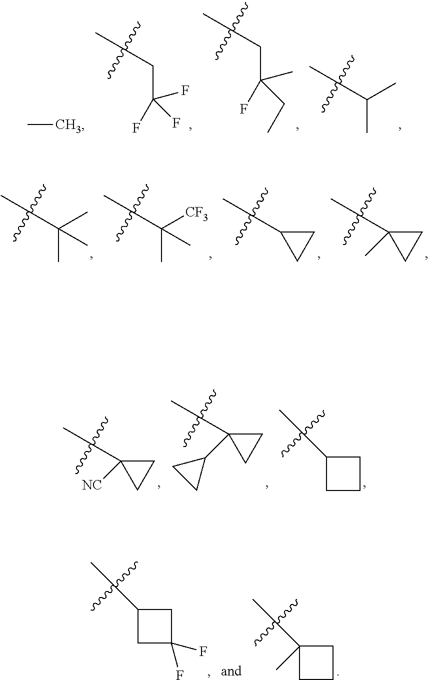

In some embodiments, R¹⁰¹ is

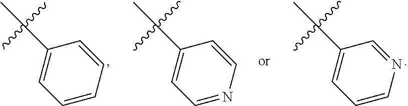 [SMILES: *c1ccccc1 ; *c1ccncc1 ; *c1cccnc1]

In some embodiments, R¹⁰⁵ is —CH₃.

In some embodiments, R¹⁰² is —NH(C═O)CF₃.

In some embodiments, R¹⁰² is

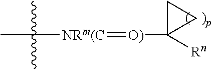

and p is selected from 1 and 2. In some embodiments, p is 1. In some embodiments, p is 2. In some embodiments, R is selected from the group consisting of halo, methyl, ethyl, propyl, —CF₂H, —CF₃, —CH₂—CF₃.

In some embodiments, R¹⁰³ is

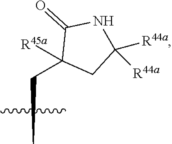

R^{44a} is each independently —CH₃, and R^{45a} is H.

In some embodiments, R¹⁰³ is

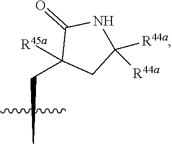

two R⁴⁴ groups, together with the carbon to which they are attached, are joined together to form a cyclopropyl, and R^{45a} is H.

In some embodiments, R¹⁰³ is

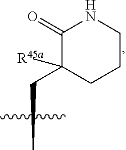

### and R⁴⁵ is H.

In some embodiments, R¹⁰³ is

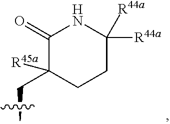

two R⁴⁴ groups, together with the carbon to which they are attached, are joined together to form a cyclopropyl, and R^{as1} is H.

In some embodiments, R¹⁰³ is

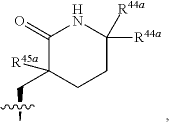

R^{44a} is H, and R^{45a} is H.

In another aspect of the disclosure, provided herein is a protease inhibitory compound represented by Formula XII:

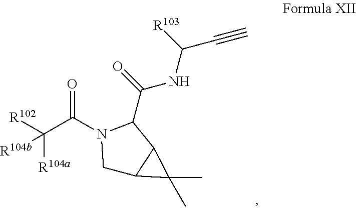

or a pharmaceutically acceptable salt, and/or a stereoisomer thereof, wherein: R^{104a} is selected from the group consisting of

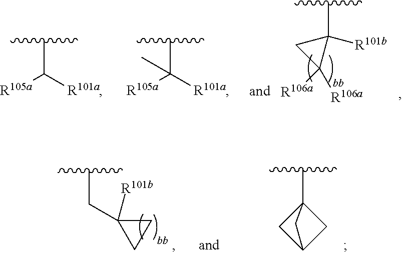

wherein R¹⁰⁵ is C₁-C₂alkyl; bb is selected from 1 and 2; R^{106a} is each independently selected from H and halo; R^{101a} and R^{101b} are each independently selected from the group consisting of H, halo, C₁-C₆alkyl, C₂-C₆alkenyl, C₃-C₆cycloalkyl, and C₃-C₆cycloalkenyl, wherein the alkyl, alkenyl, cycloalkyl, or cycloalkenyl may optionally be substituted by one, two, three, or four substituents each independently selected from the group consisting of halogen, C₁-C₆alkyl, C₁-C₆alkoxy, C₃-C₆cycloalkyl, phenyl, and pyridinyl, and cyano, wherein the alkyl may optionally be substituted by one or more halo; R^{104b} represents H;
alternatively, R^{104a} and R^{104b}, together with the carbon atom to which they are attached, may be joined to form a C₃-C₆cycloalkyl, wherein the cycloalkyl ring may be optionally substituted by one, two, three, or four substituents each independently selected from R^{P}; wherein R^{P} is selected from the group consisting of halogen, C₁-C₆alkyl, C₁-C₆alkenyl, C₁-C₆alkoxy, C₃-C₆cycloalkyl, and cyano, wherein the alkyl may optionally be substituted by one or more halo; R¹°² is selected from the group consisting of —NR^{m}(C═O)Rⁿ,

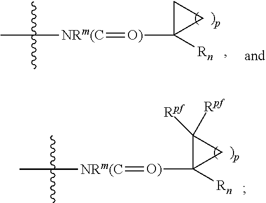

p is selected from 1 and 2; R^{m} is H; Rⁿ is selected from C₁₋₂alkyl and C₃-C₄cycloalkyl, wherein the alkyl or cycloalkyl is optionally substituted by one, two or three methyl, halo or —CF₃; R^{pf} is each independently selected from H and halo; R¹⁰³ is selected from

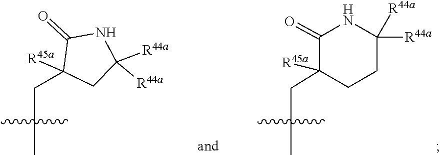

R^{44a} is each independently selected from the group consisting of hydrogen, —CH₃ and —CF₃, or two R^{44a} groups, together with the carbon to which they are attached, may be joined together to form a cyclopropyl, cyclobutyl, or oxetanyl; and R^{45a} is each independently selected from the group consisting of H, CH₃ and halo.

In some embodiments, R¹⁰³ is selected from

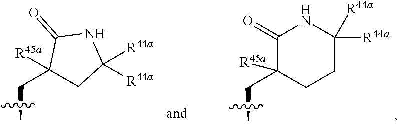

In some embodiments, Formula X is represented by a formula selected from the group consisting of

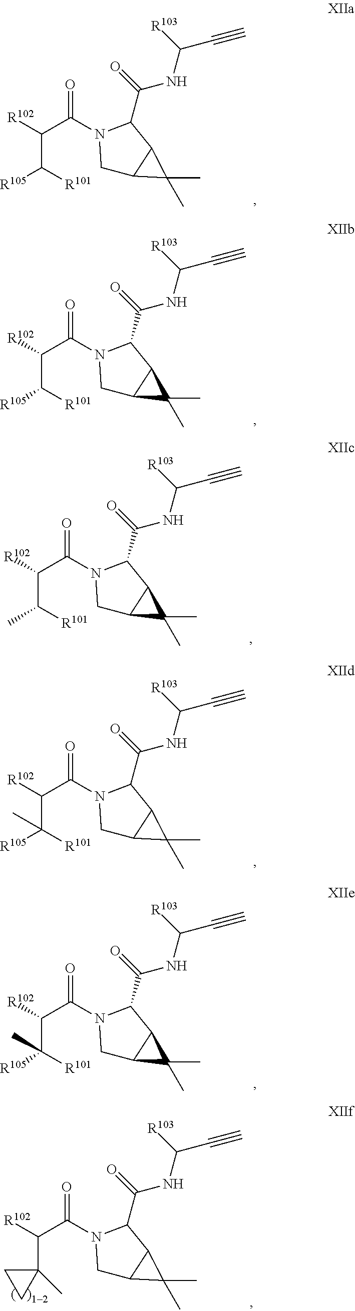

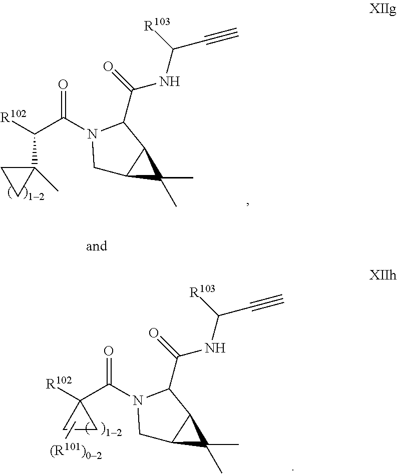

In some embodiments, R^{104a} is

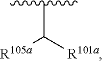

R^{101a} is C₁-C₃alkyl or C₃-C₄cycloalkyl, and R^{105a} is C₁-C₂alkyl, wherein the R¹⁰¹ is optionally substituted by one or two substituents each independently selected from the group consisting of —F, —CF₃, —CH₃, —CN, cyclopropyl, phenyl and pyridyl.

In some embodiments, R^{104a} is

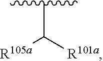

R^{101a} is C₁-C₃alkyl or C₃-C₄cycloalkyl, and R^{105a} is C₁-C₂alkyl, wherein the R¹⁰¹ is optionally substituted by one or two substituents each independently selected from the group consisting of —F, —CF₃, —CH₃, —CN and cyclopropyl.

In some embodiments,

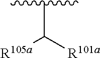

is selected from the group consisting of

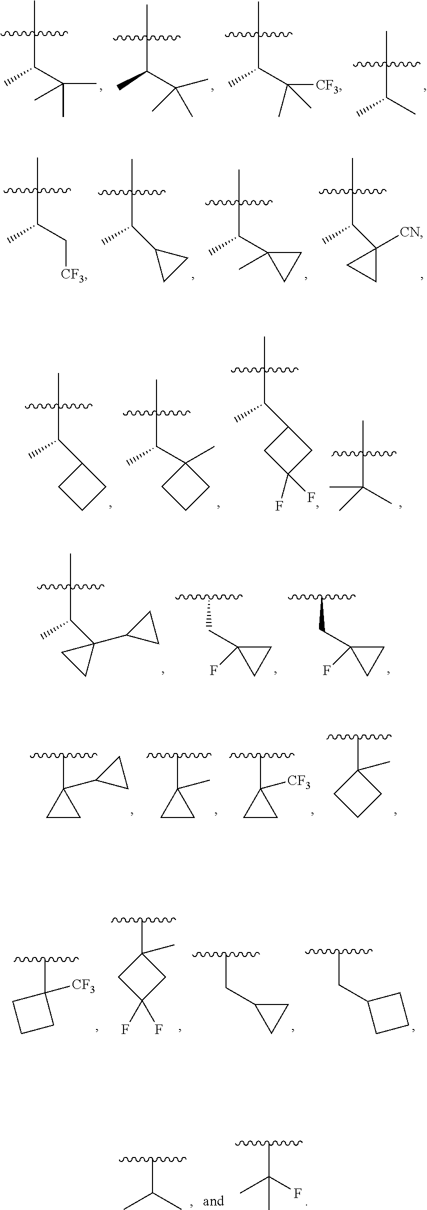

In some embodiments,

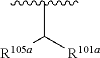

is selected from the group consisting of

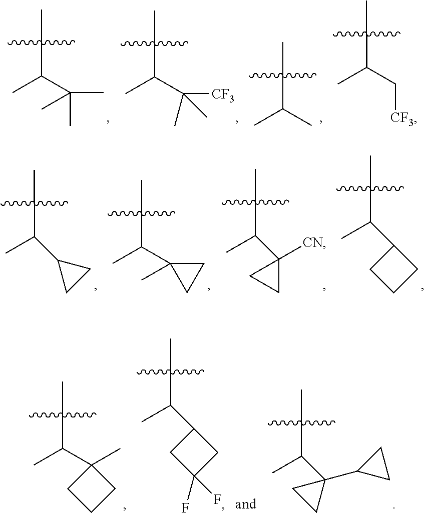

In some embodiments, R^{104a} is

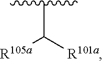

R^{101a} is C₁-C₆alkyl or C₃-C₆cycloalkyl, and R^{105a} is C₁-C₂alkyl, wherein the R¹⁰¹ is optionally substituted by one or two substituents each independently selected from the group consisting of —F, —CF₃, —CH₃, —CN, phenyl, pyridinyl and cyclopropyl.

In some embodiments, R^{104a} is

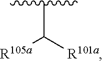

R^{101a} is C₁-C₆alkyl or C₃-C₆cycloalkyl, and R^{105a} is C₁-C₂alkyl, wherein the R¹⁰¹ is optionally substituted by one or two substituents each independently selected from the group consisting of —F, —CF₃, —CH₃, —CN and cyclopropyl.

In some embodiments,

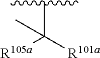

is

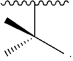

In some embodiments,

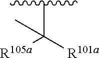

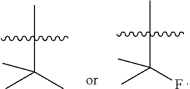

In some embodiments, R^{101a} is selected from the group consisting of:

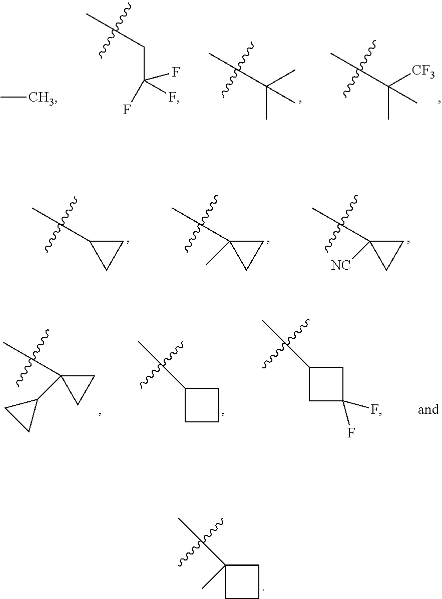

In some embodiments, R¹⁰¹ is

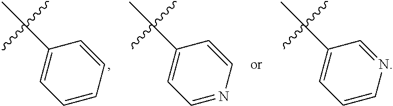 [SMILES: *c1ccccc1 ; *c1ccncc1 ; *c1cccnc1]

In some embodiments, R^{103a} is —CH₃.

In some embodiments, R^{104a} is selected from the group consisting of

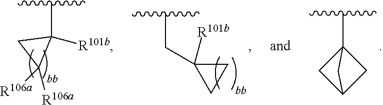

In some embodiments, bb is 1. In some embodiments, bb is 2. In some embodiments, R^{101b} is H. In some embodiments, R^{101b} is CH₃. In some embodiments, R^{106a} is H. In some embodiments, R^{106a} is halo. In some embodiments, R^{106a} is F.

In some embodiments, R¹⁰² is —NH(C═O)CF₃.

In some embodiments, R¹⁰² is

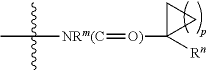

and p is selected from 1 and 2.

In some embodiments, R¹⁰² is

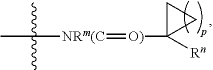

R^{m} is H, and p is selected from 1 and 2. In some embodiments, p is 1. In some embodiments, p is 2.

In some embodiments, R¹⁰² is

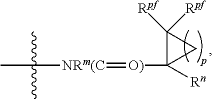

R^{m} is H, p is selected from 1 and 2, and R¹¹ is each independently H or halo. In some embodiments, R¹⁰² is

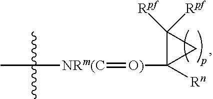

R^{m} is H, p is 1 or 2; and R^{pf} is halo. In some embodiments, R¹⁰² is

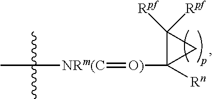

R^{m} is H, p is 1 and R^{pf} is halo. In some embodiments, R¹⁰² is

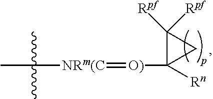

R^{m} is H, p is 2 and R¹¹ is halo. In some embodiments, R^{pf} is F.

In some embodiments, R¹⁰³ is

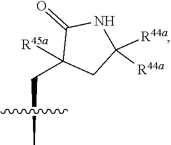

R^{44a} is each independently —CH₃, and R^{45a} is H.

In some embodiments, R¹⁰³ is

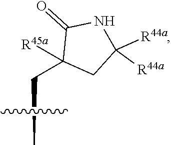

two R^{44a} groups, together with the carbon to which they are attached, are joined together to form a cyclopropyl, and R^{as} is H.

In some embodiments, R¹⁰³ is

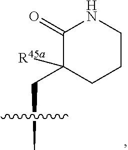

### and R^{45a} is H.

In some embodiments, R¹⁰³ is

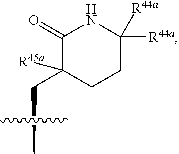

two R^{44a} groups, together with the carbon to which they are attached, are joined together to for m a cyclopropyl, and R^{45a} is H.

In some embodiments, R¹⁰³ is

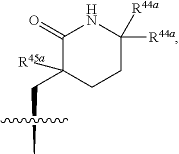

R^{44a} is H, and R^{45a} is H.

In embodiments, a disclosed compound is selected from the group consisting of

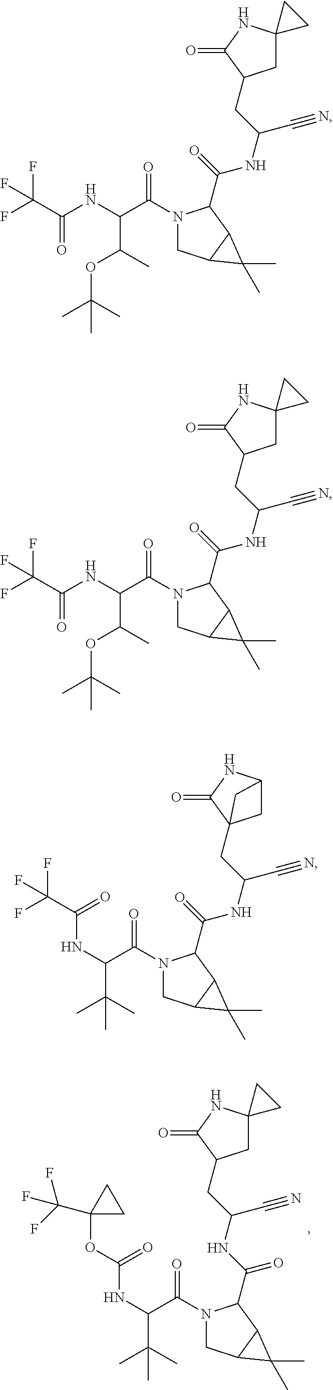

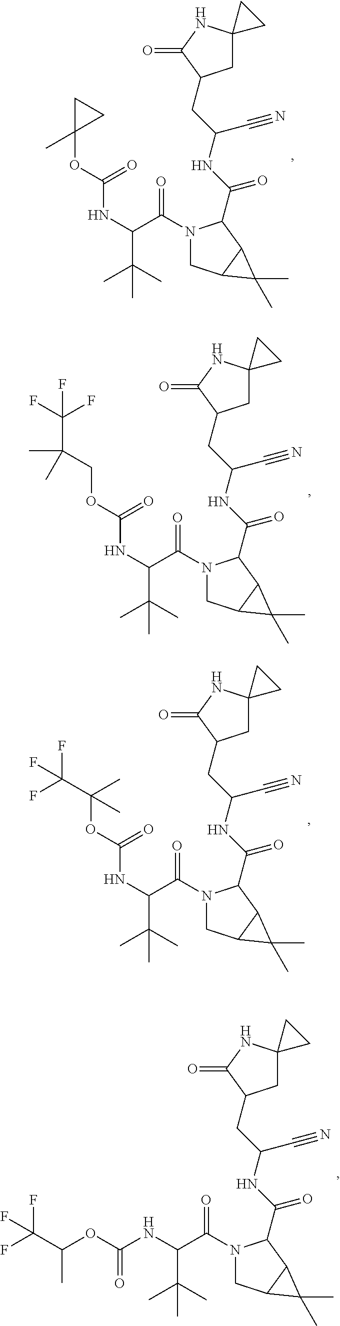

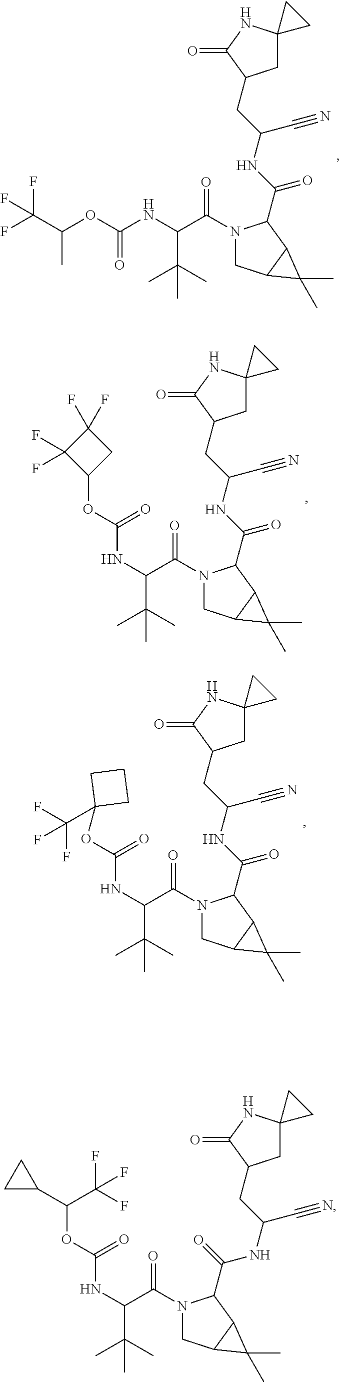

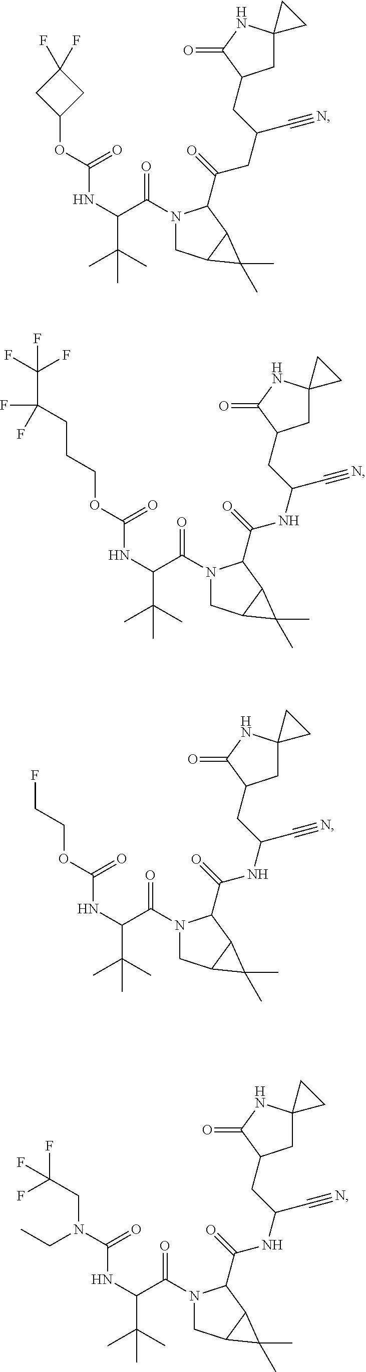 [SMILES: CC(C)(C)C(NC(=O)OC1CC(F)(F)C1)C(=O)N1CC2C(C1C(=O)CC(C#N)CC1CC3(CC3)NC1=O)C2(C)C ; CC(C)(C)C(NC(=O)OCCCC(F)(F)C(F)(F)F)C(=O)N1CC2C(C1C(=O)NC(C#N)CC1CC3(CC3)NC1=O)C2(C)C ; CC(C)(C)C(NC(=O)OCCF)C(=O)N1CC2C(C1C(=O)NC(C#N)CC1CC3(CC3)NC1=O)C2(C)C ; CCN(CC(F)(F)F)C(=O)NC(C(=O)N1CC2C(C1C(=O)NC(C#N)CC1CC3(CC3)NC1=O)C2(C)C)C(C)(C)C]

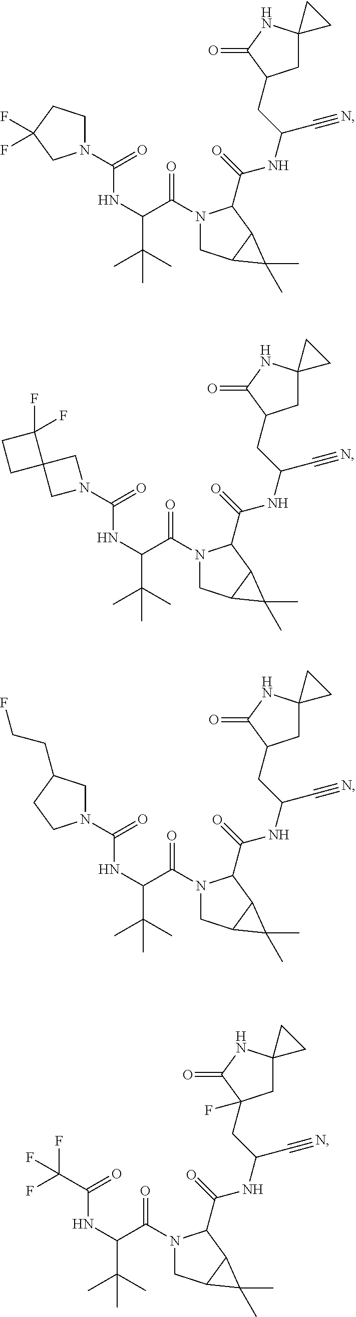 [SMILES: CC(C)(C)C(NC(=O)N1CCC(F)(F)C1)C(=O)N1CC2C(C1C(=O)NC(C#N)CC1CC3(CC3)NC1=O)C2(C)C ; CC(C)(C)C(NC(=O)N1CC2(CCC2(F)F)C1)C(=O)N1CC2C(C1C(=O)NC(C#N)CC1CC3(CC3)NC1=O)C2(C)C ; CC(C)(C)C(NC(=O)N1CCC(CCF)C1)C(=O)N1CC2C(C1C(=O)NC(C#N)CC1CC3(CC3)NC1=O)C2(C)C ; CC(C)(C)C(NC(=O)C(F)(F)F)C(=O)N1CC2C(C1C(=O)NC(C#N)CC1(F)CC3(CC3)NC1=O)C2(C)C]

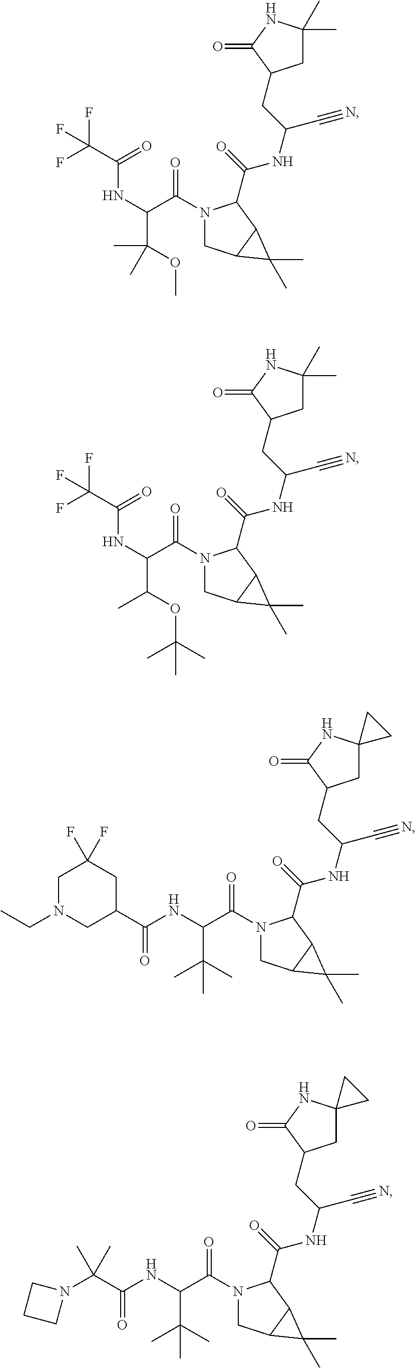 [SMILES: COC(C)(C)C(NC(=O)C(F)(F)F)C(=O)N1CC2C(C1C(=O)NC(C#N)CC1CC(C)(C)NC1=O)C2(C)C ; CC(OC(C)(C)C)C(NC(=O)C(F)(F)F)C(=O)N1CC2C(C1C(=O)NC(C#N)CC1CC(C)(C)NC1=O)C2(C)C ; CCN1CC(C(=O)NC(C(=O)N2CC3C(C2C(=O)NC(C#N)CC2CC4(CC4)NC2=O)C3(C)C)C(C)(C)C)CC(F)(F)C1 ; CC(C)(C)C(NC(=O)C(C)(C)N1CCC1)C(=O)N1CC2C(C1C(=O)NC(C#N)CC1CC3(CC3)NC1=O)C2(C)C]

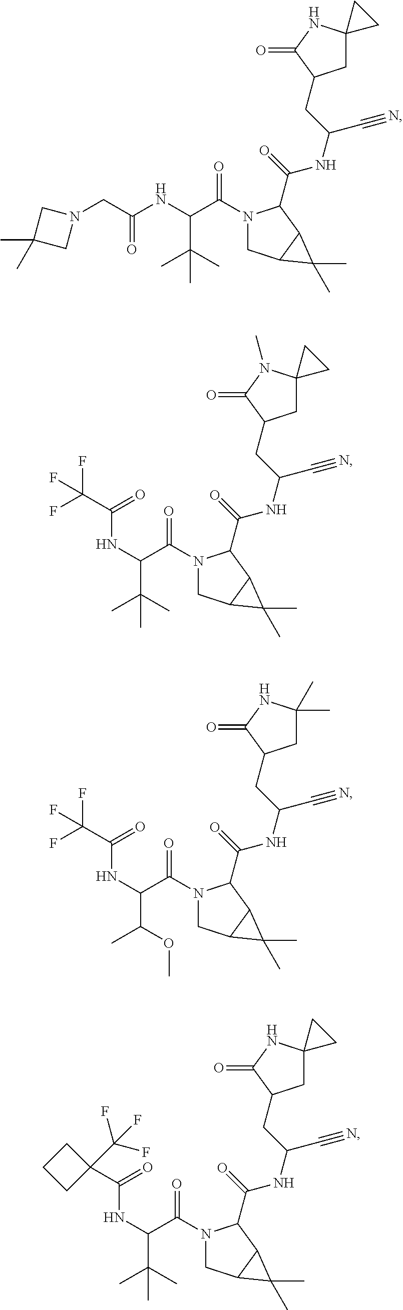 [SMILES: CC1(C)CN(CC(=O)NC(C(=O)N2CC3C(C2C(=O)NC(C#N)CC2CC4(CC4)NC2=O)C3(C)C)C(C)(C)C)C1 ; CN1C(=O)C(CC(C#N)NC(=O)C2C3C(CN2C(=O)C(NC(=O)C(F)(F)F)C(C)(C)C)C3(C)C)CC12CC2 ; COC(C)C(NC(=O)C(F)(F)F)C(=O)N1CC2C(C1C(=O)NC(C#N)CC1CC(C)(C)NC1=O)C2(C)C ; CC(C)(C)C(NC(=O)C1(C(F)(F)F)CCC1)C(=O)N1CC2C(C1C(=O)NC(C#N)CC1CC3(CC3)NC1=O)C2(C)C]

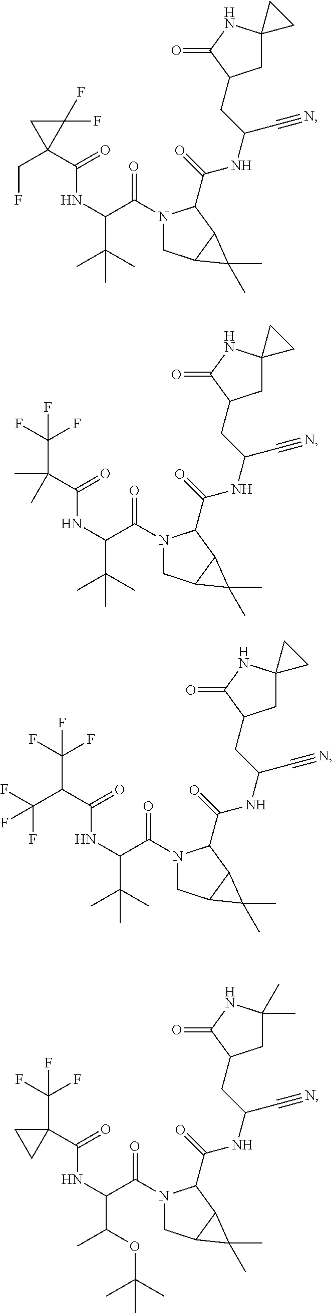

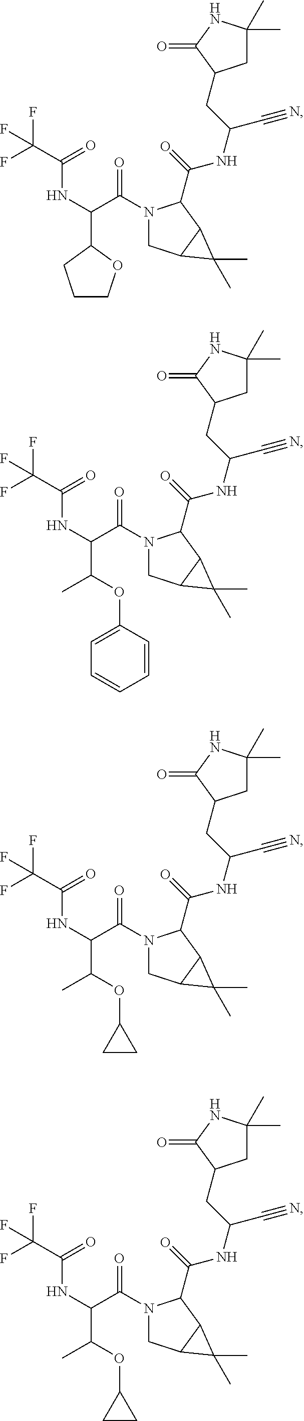 [SMILES: CC1(C)CC(CC(C#N)NC(=O)C2C3C(CN2C(=O)C(NC(=O)C(F)(F)F)C2CCCO2)C3(C)C)C(=O)N1 ; CC(Oc1ccccc1)C(NC(=O)C(F)(F)F)C(=O)N1CC2C(C1C(=O)NC(C#N)CC1CC(C)(C)NC1=O)C2(C)C ; CC(OC1CC1)C(NC(=O)C(F)(F)F)C(=O)N1CC2C(C1C(=O)NC(C#N)CC1CC(C)(C)NC1=O)C2(C)C ; CC(OC1CC1)C(NC(=O)C(F)(F)F)C(=O)N1CC2C(C1C(=O)NC(C#N)CC1CC(C)(C)NC1=O)C2(C)C]

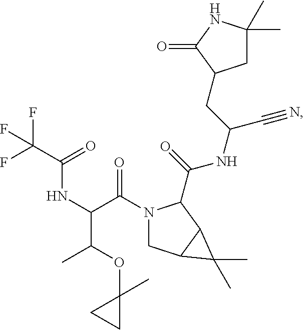 [SMILES: CC(OC1(C)CC1)C(NC(=O)C(F)(F)F)C(=O)N1CC2C(C1C(=O)NC(C#N)CC1CC(C)(C)NC1=O)C2(C)C]

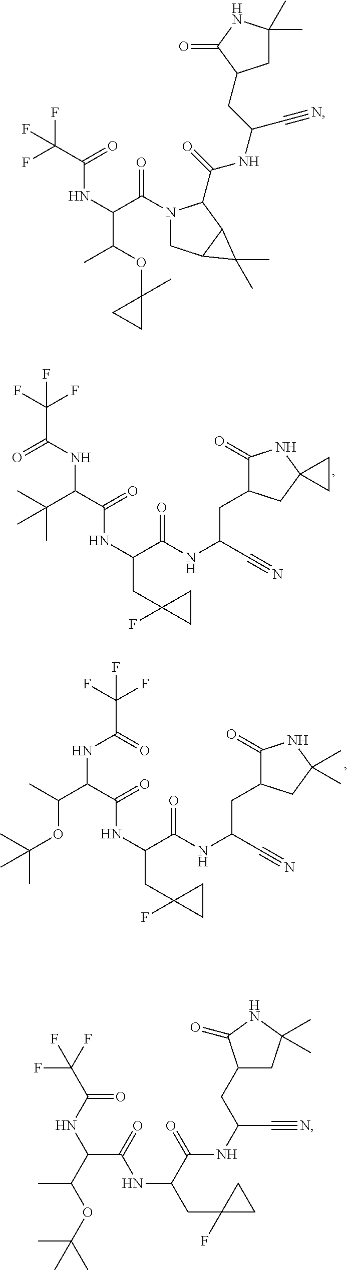

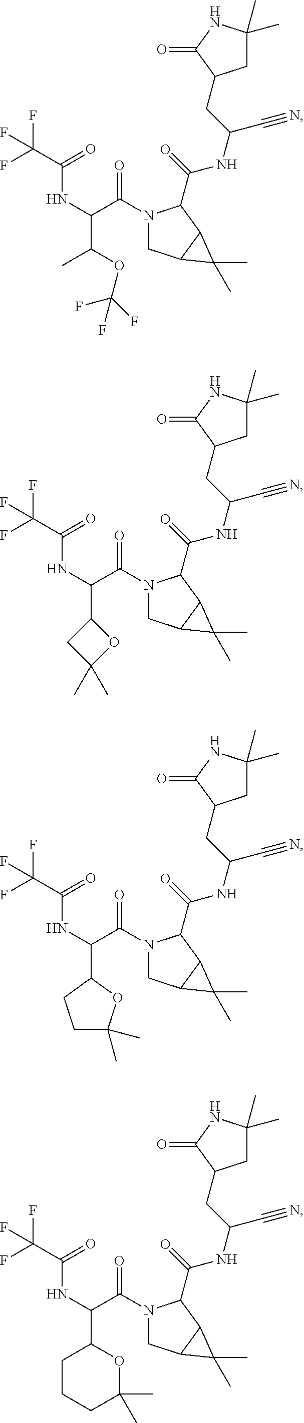 [SMILES: CC(OC(F)(F)F)C(NC(=O)C(F)(F)F)C(=O)N1CC2C(C1C(=O)NC(C#N)CC1CC(C)(C)NC1=O)C2(C)C ; CC1(C)CC(CC(C#N)NC(=O)C2C3C(CN2C(=O)C(NC(=O)C(F)(F)F)C2CC(C)(C)O2)C3(C)C)C(=O)N1 ; CC1(C)CC(CC(C#N)NC(=O)C2C3C(CN2C(=O)C(NC(=O)C(F)(F)F)C2CCC(C)(C)O2)C3(C)C)C(=O)N1 ; CC1(C)CC(CC(C#N)NC(=O)C2C3C(CN2C(=O)C(NC(=O)C(F)(F)F)C2CCCC(C)(C)O2)C3(C)C)C(=O)N1]

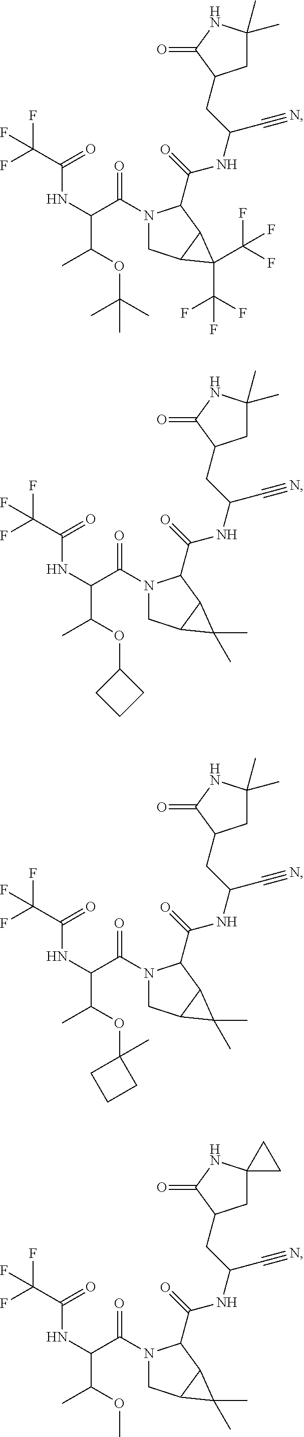 [SMILES: CC(OC(C)(C)C)C(NC(=O)C(F)(F)F)C(=O)N1CC2C(C1C(=O)NC(C#N)CC1CC(C)(C)NC1=O)C2(C(F)(F)F)C(F)(F)F ; CC(OC1CCC1)C(NC(=O)C(F)(F)F)C(=O)N1CC2C(C1C(=O)NC(C#N)CC1CC(C)(C)NC1=O)C2(C)C ; CC(OC1(C)CCC1)C(NC(=O)C(F)(F)F)C(=O)N1CC2C(C1C(=O)NC(C#N)CC1CC(C)(C)NC1=O)C2(C)C ; COC(C)C(NC(=O)C(F)(F)F)C(=O)N1CC2C(C1C(=O)NC(C#N)CC1CC3(CC3)NC1=O)C2(C)C]

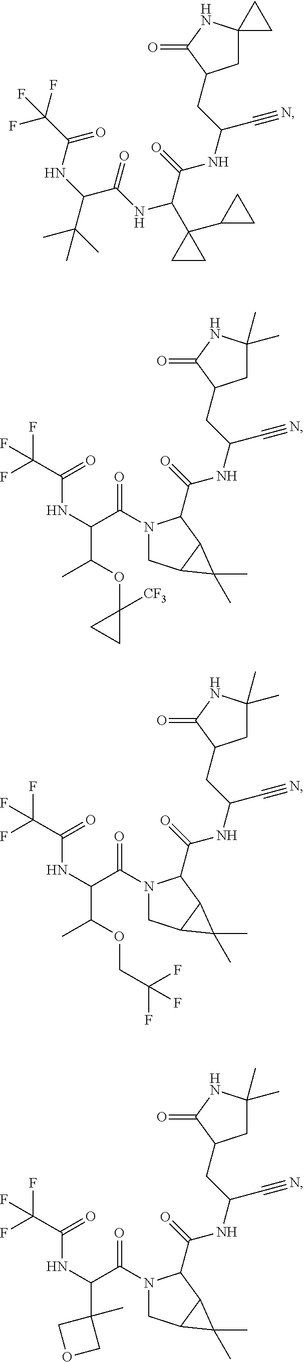 [SMILES: CC(C)(C)C(NC(=O)C(F)(F)F)C(=O)NC(C(=O)NC(C#N)CC1CC2(CC2)NC1=O)C1(C2CC2)CC1 ; CC(OC1(C(F)(F)F)CC1)C(NC(=O)C(F)(F)F)C(=O)N1CC2C(C1C(=O)NC(C#N)CC1CC(C)(C)NC1=O)C2(C)C ; CC(OCC(F)(F)F)C(NC(=O)C(F)(F)F)C(=O)N1CC2C(C1C(=O)NC(C#N)CC1CC(C)(C)NC1=O)C2(C)C ; CC1(C)CC(CC(C#N)NC(=O)C2C3C(CN2C(=O)C(NC(=O)C(F)(F)F)C2(C)COC2)C3(C)C)C(=O)N1]

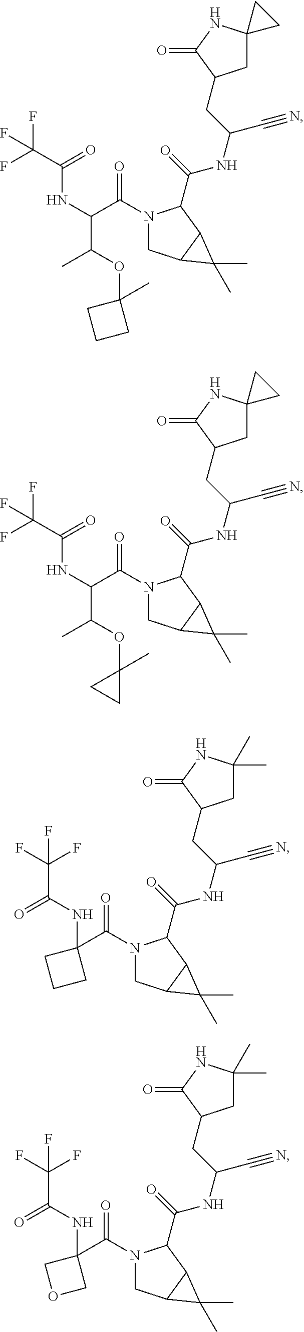

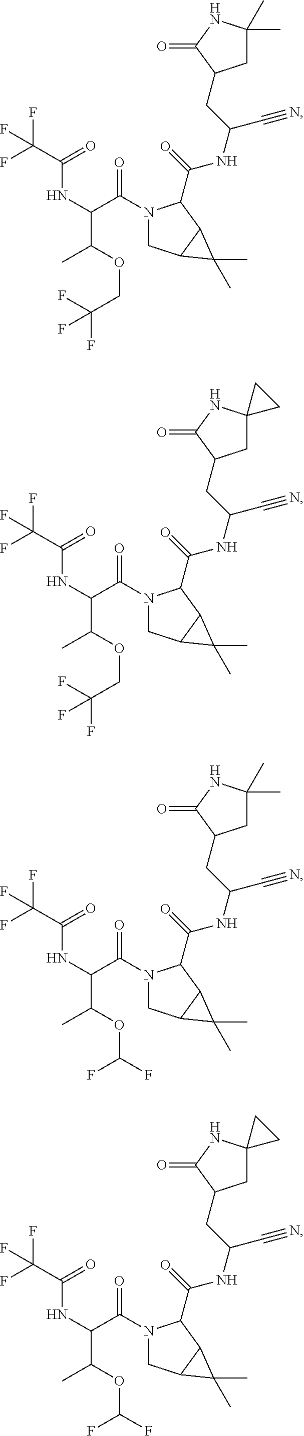 [SMILES: CC(OCC(F)(F)F)C(NC(=O)C(F)(F)F)C(=O)N1CC2C(C1C(=O)NC(C#N)CC1CC(C)(C)NC1=O)C2(C)C ; CC(OCC(F)(F)F)C(NC(=O)C(F)(F)F)C(=O)N1CC2C(C1C(=O)NC(C#N)CC1CC3(CC3)NC1=O)C2(C)C ; CC(OC(F)F)C(NC(=O)C(F)(F)F)C(=O)N1CC2C(C1C(=O)NC(C#N)CC1CC(C)(C)NC1=O)C2(C)C ; CC(OC(F)F)C(NC(=O)C(F)(F)F)C(=O)N1CC2C(C1C(=O)NC(C#N)CC1CC3(CC3)NC1=O)C2(C)C]

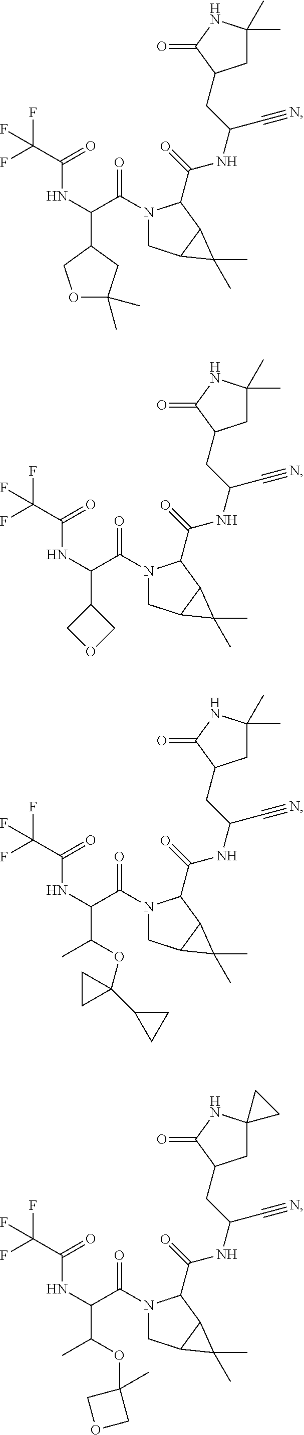 [SMILES: CC1(C)CC(CC(C#N)NC(=O)C2C3C(CN2C(=O)C(NC(=O)C(F)(F)F)C2COC(C)(C)C2)C3(C)C)C(=O)N1 ; CC1(C)CC(CC(C#N)NC(=O)C2C3C(CN2C(=O)C(NC(=O)C(F)(F)F)C2COC2)C3(C)C)C(=O)N1 ; CC(OC1(C2CC2)CC1)C(NC(=O)C(F)(F)F)C(=O)N1CC2C(C1C(=O)NC(C#N)CC1CC(C)(C)NC1=O)C2(C)C ; CC(OC1(C)COC1)C(NC(=O)C(F)(F)F)C(=O)N1CC2C(C1C(=O)NC(C#N)CC1CC3(CC3)NC1=O)C2(C)C]

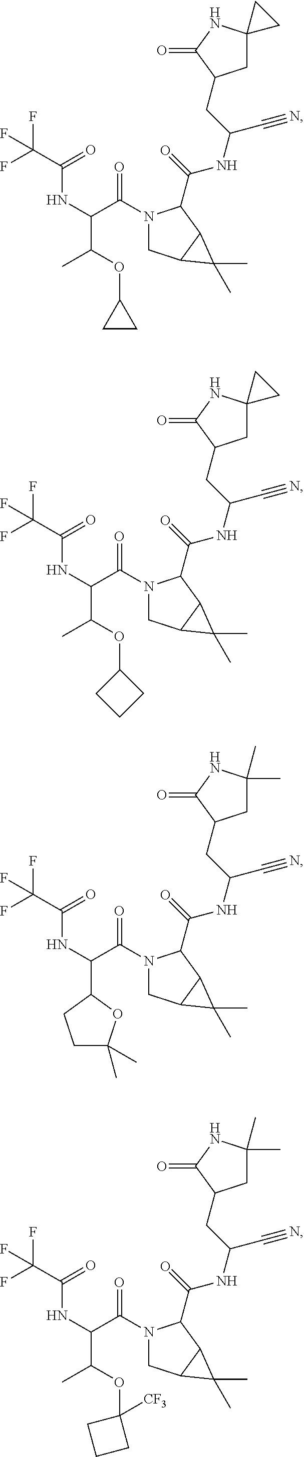 [SMILES: CC(OC1CC1)C(NC(=O)C(F)(F)F)C(=O)N1CC2C(C1C(=O)NC(C#N)CC1CC3(CC3)NC1=O)C2(C)C ; CC(OC1CCC1)C(NC(=O)C(F)(F)F)C(=O)N1CC2C(C1C(=O)NC(C#N)CC1CC3(CC3)NC1=O)C2(C)C ; CC1(C)CC(CC(C#N)NC(=O)C2C3C(CN2C(=O)C(NC(=O)C(F)(F)F)C2CCC(C)(C)O2)C3(C)C)C(=O)N1 ; CC(OC1(C(F)(F)F)CCC1)C(NC(=O)C(F)(F)F)C(=O)N1CC2C(C1C(=O)NC(C#N)CC1CC(C)(C)NC1=O)C2(C)C]

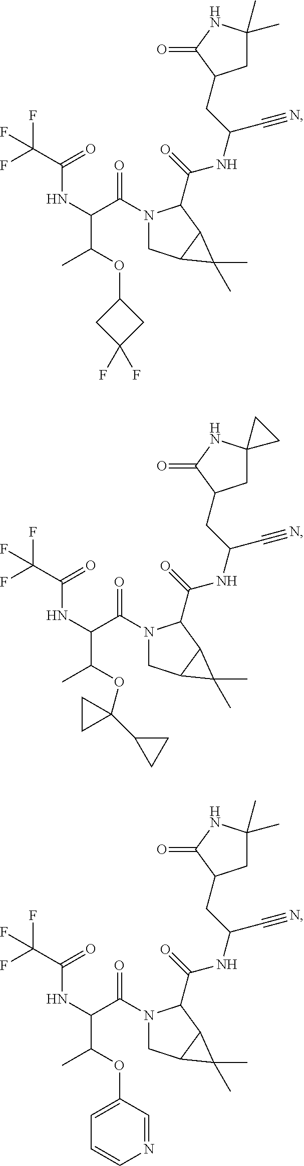 [SMILES: CC(OC1CC(F)(F)C1)C(NC(=O)C(F)(F)F)C(=O)N1CC2C(C1C(=O)NC(C#N)CC1CC(C)(C)NC1=O)C2(C)C ; CC(OC1(C2CC2)CC1)C(NC(=O)C(F)(F)F)C(=O)N1CC2C(C1C(=O)NC(C#N)CC1CC3(CC3)NC1=O)C2(C)C ; CC(Oc1cccnc1)C(NC(=O)C(F)(F)F)C(=O)N1CC2C(C1C(=O)NC(C#N)CC1CC(C)(C)NC1=O)C2(C)C]

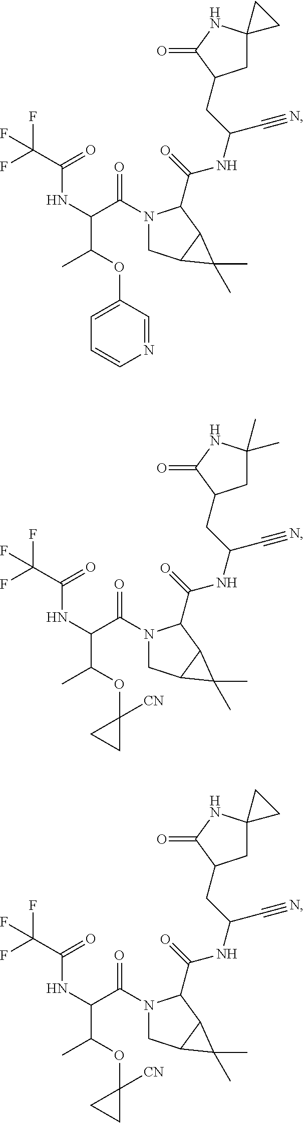 [SMILES: CC(Oc1cccnc1)C(NC(=O)C(F)(F)F)C(=O)N1CC2C(C1C(=O)NC(C#N)CC1CC3(CC3)NC1=O)C2(C)C ; CC(OC1(C#N)CC1)C(NC(=O)C(F)(F)F)C(=O)N1CC2C(C1C(=O)NC(C#N)CC1CC(C)(C)NC1=O)C2(C)C ; CC(OC1(C#N)CC1)C(NC(=O)C(F)(F)F)C(=O)N1CC2C(C1C(=O)NC(C#N)CC1CC3(CC3)NC1=O)C2(C)C]

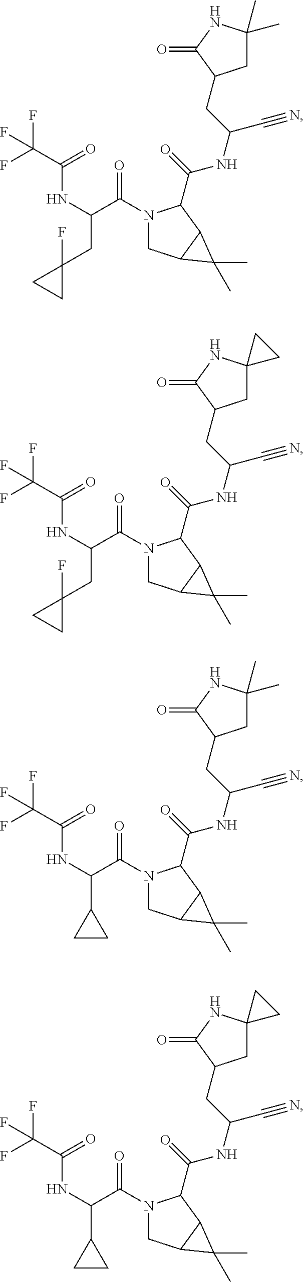

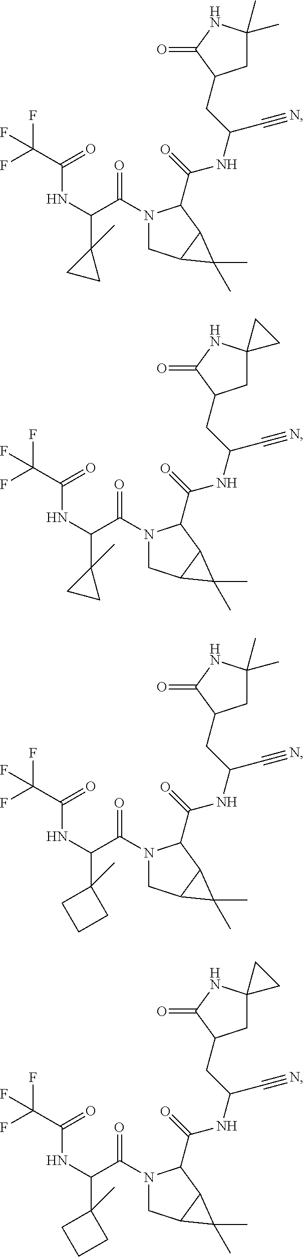

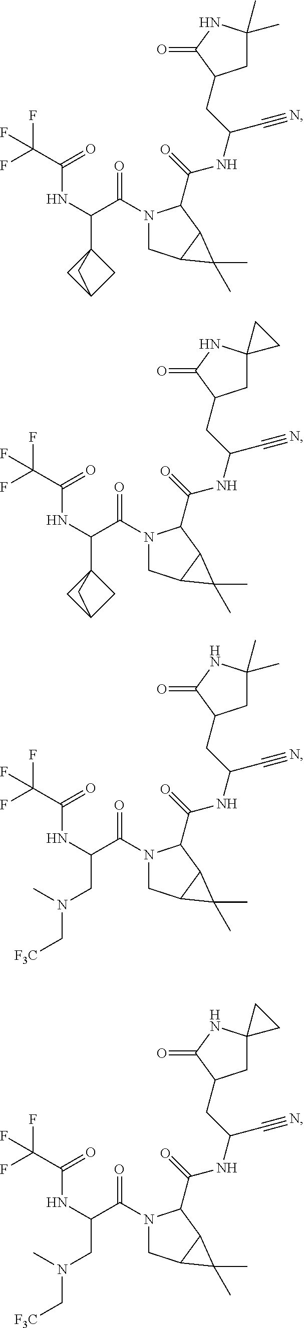

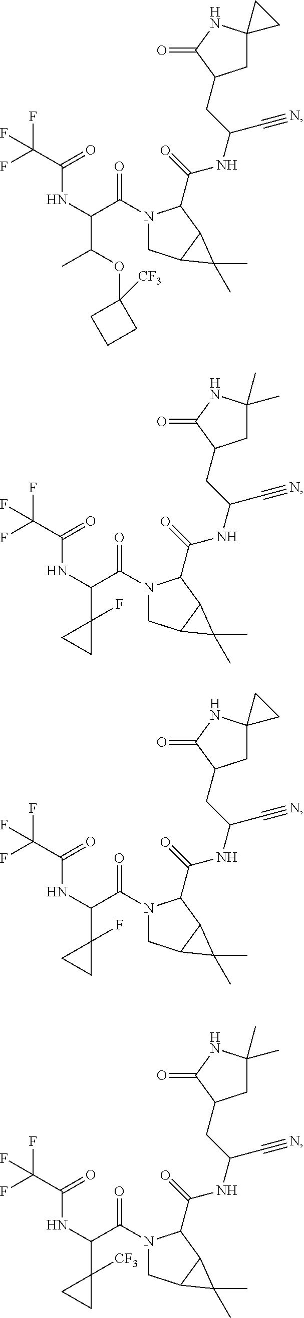

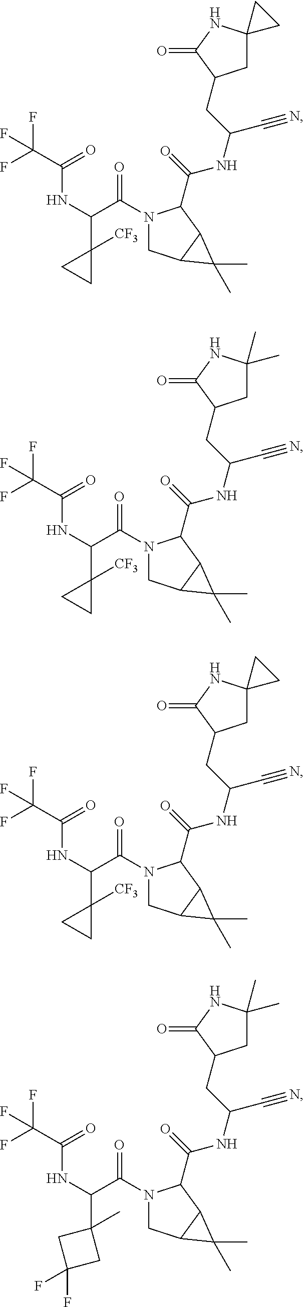

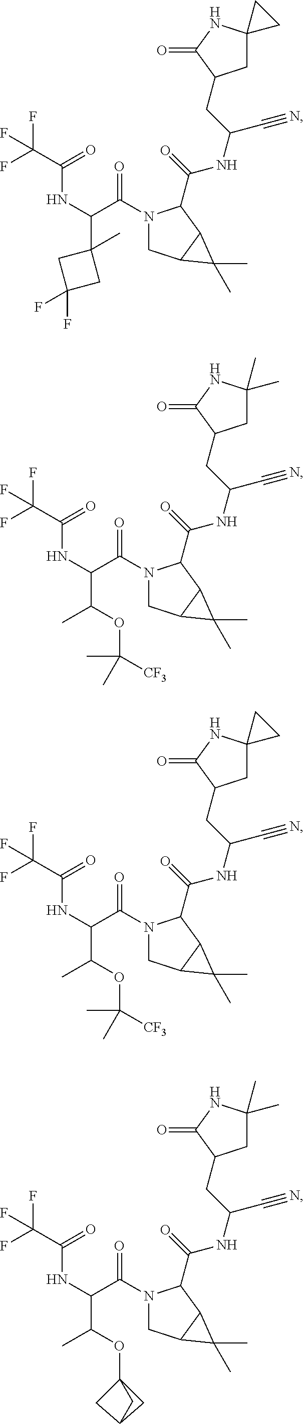 [SMILES: CC1(C(NC(=O)C(F)(F)F)C(=O)N2CC3C(C2C(=O)NC(C#N)CC2CC4(CC4)NC2=O)C3(C)C)CC(F)(F)C1 ; CC(OC(C)(C)C(F)(F)F)C(NC(=O)C(F)(F)F)C(=O)N1CC2C(C1C(=O)NC(C#N)CC1CC(C)(C)NC1=O)C2(C)C ; CC(OC(C)(C)C(F)(F)F)C(NC(=O)C(F)(F)F)C(=O)N1CC2C(C1C(=O)NC(C#N)CC1CC3(CC3)NC1=O)C2(C)C ; CC(OC12CC(C1)C2)C(NC(=O)C(F)(F)F)C(=O)N1CC2C(C1C(=O)NC(C#N)CC1CC(C)(C)NC1=O)C2(C)C]

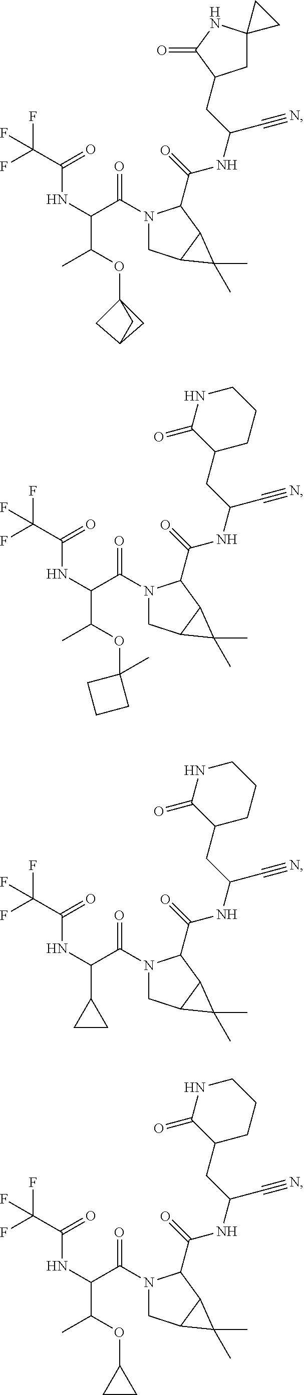

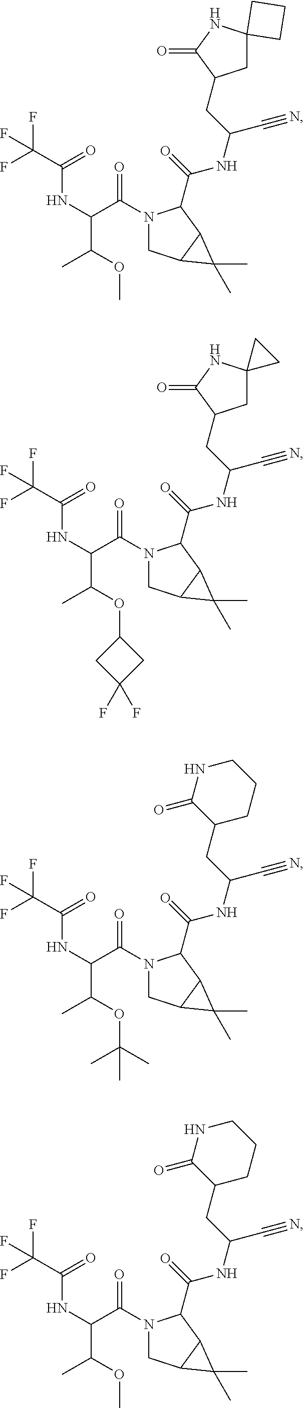 [SMILES: COC(C)C(NC(=O)C(F)(F)F)C(=O)N1CC2C(C1C(=O)NC(C#N)CC1CC3(CCC3)NC1=O)C2(C)C ; CC(OC1CC(F)(F)C1)C(NC(=O)C(F)(F)F)C(=O)N1CC2C(C1C(=O)NC(C#N)CC1CC3(CC3)NC1=O)C2(C)C ; CC(OC(C)(C)C)C(NC(=O)C(F)(F)F)C(=O)N1CC2C(C1C(=O)NC(C#N)CC1CCCNC1=O)C2(C)C ; COC(C)C(NC(=O)C(F)(F)F)C(=O)N1CC2C(C1C(=O)NC(C#N)CC1CCCNC1=O)C2(C)C]

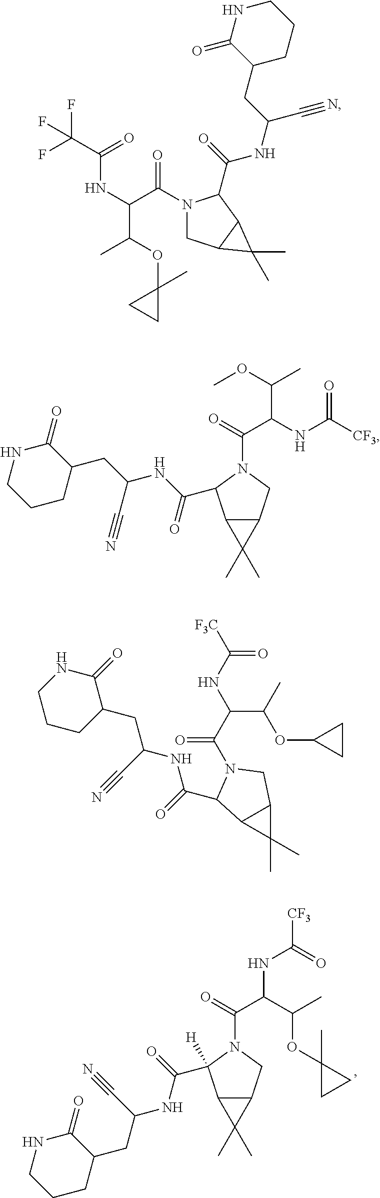 [SMILES: CC(OC1(C)CC1)C(NC(=O)C(F)(F)F)C(=O)N1CC2C(C1C(=O)NC(C#N)CC1CCCNC1=O)C2(C)C ; COC(C)C(NC(=O)C(F)(F)F)C(=O)N1CC2C(C1C(=O)NC(C#N)CC1CCCNC1=O)C2(C)C ; CC(OC1CC1)C(NC(=O)C(F)(F)F)C(=O)N1CC2C(C1C(=O)NC(C#N)CC1CCCNC1=O)C2(C)C ; CC(OC1(C)CC1)C(NC(=O)C(F)(F)F)C(=O)N1CC2C([C@H]1C(=O)NC(C#N)CC1CCCNC1=O)C2(C)C]

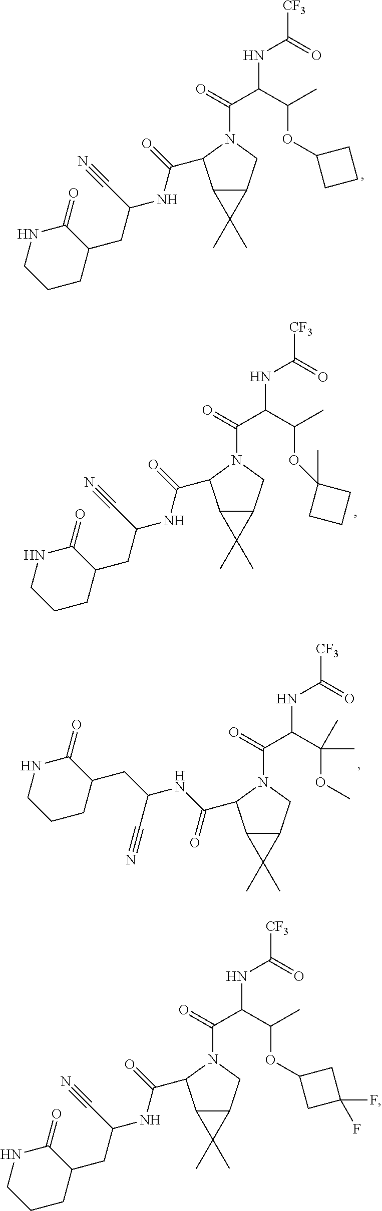

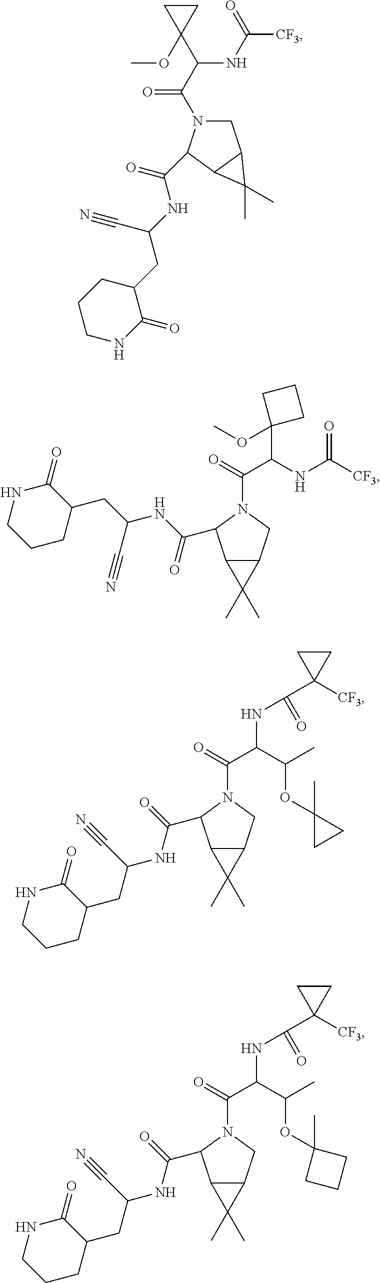 [SMILES: COC1(C(NC(=O)C(F)(F)F)C(=O)N2CC3C(C2C(=O)NC(C#N)CC2CCCNC2=O)C3(C)C)CC1 ; COC1(C(NC(=O)C(F)(F)F)C(=O)N2CC3C(C2C(=O)NC(C#N)CC2CCCNC2=O)C3(C)C)CCC1 ; CC(OC1(C)CC1)C(NC(=O)C1(C(F)(F)F)CC1)C(=O)N1CC2C(C1C(=O)NC(C#N)CC1CCCNC1=O)C2(C)C ; CC(OC1(C)CCC1)C(NC(=O)C1(C(F)(F)F)CC1)C(=O)N1CC2C(C1C(=O)NC(C#N)CC1CCCNC1=O)C2(C)C]

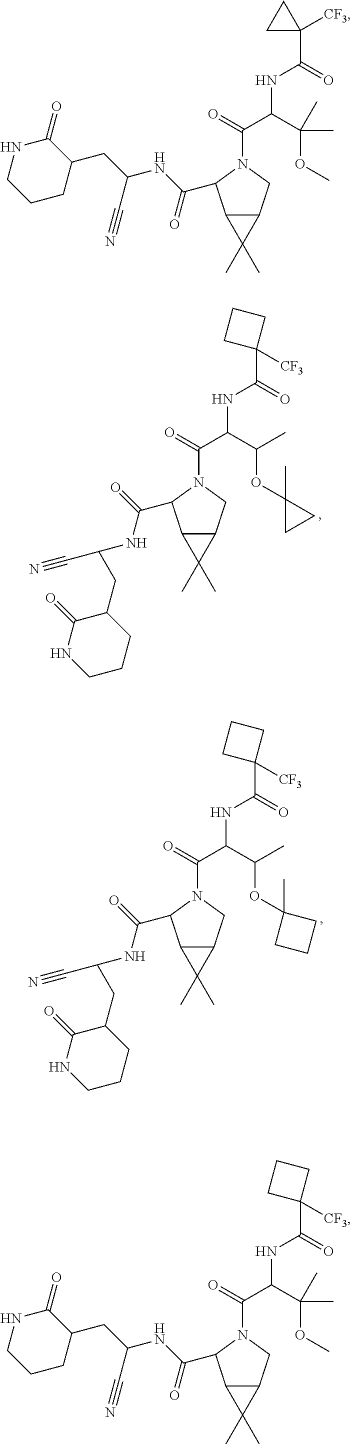

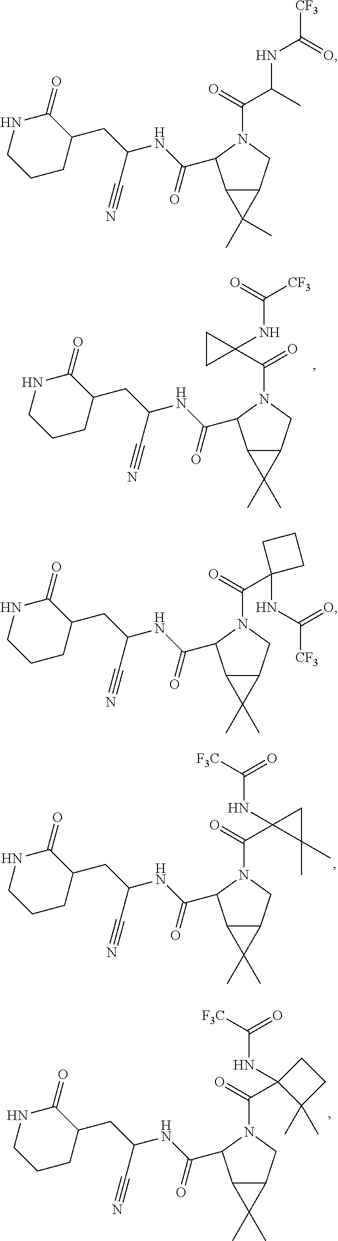

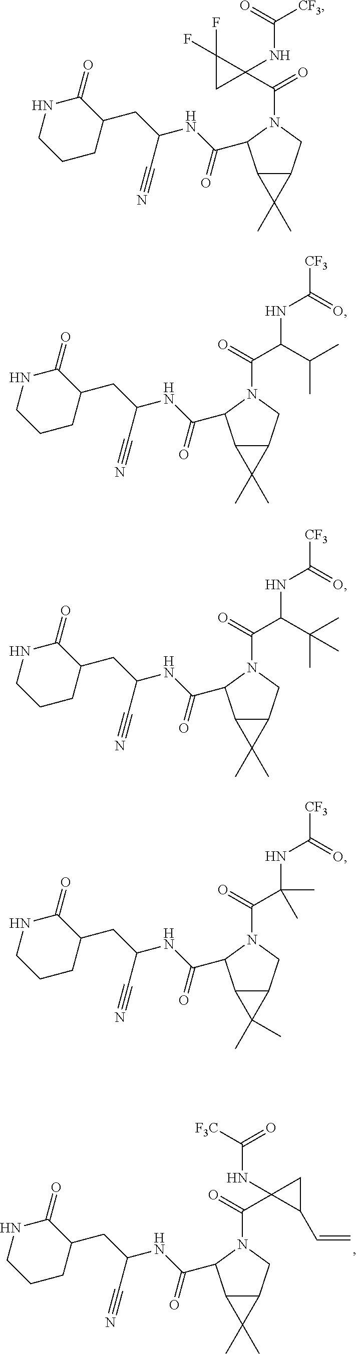

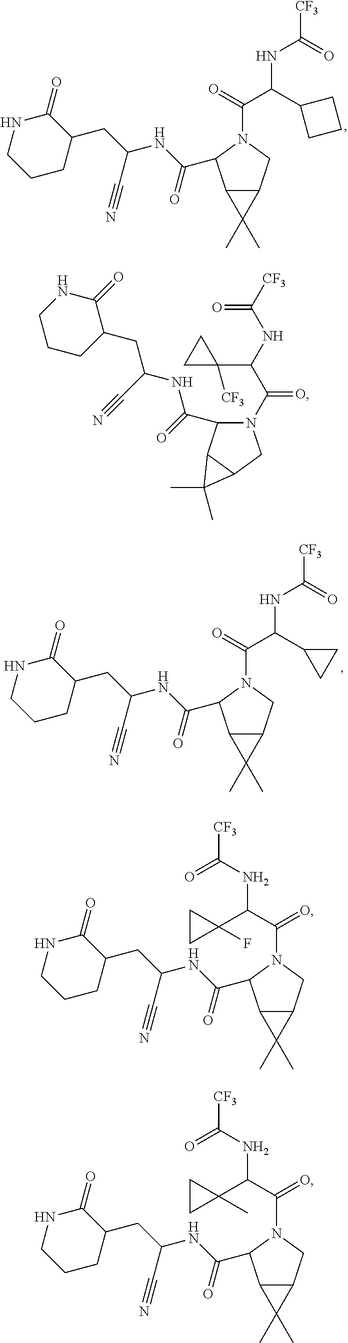

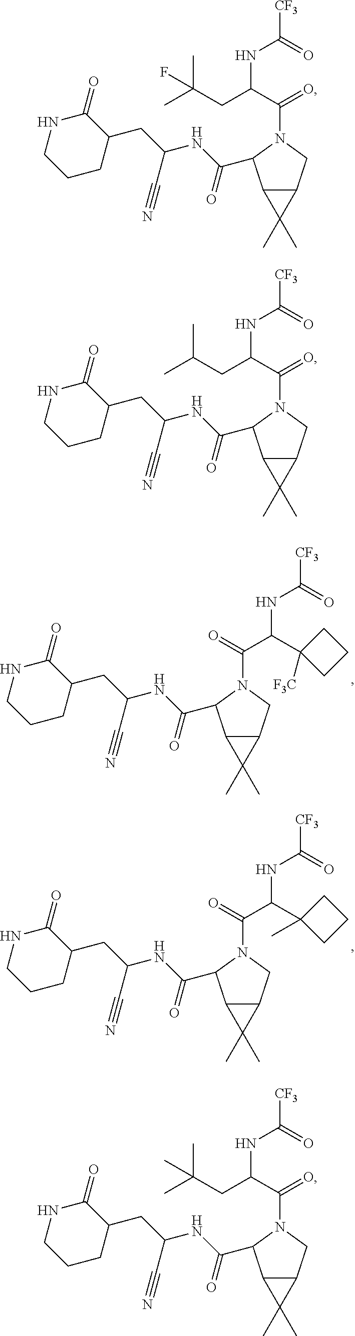

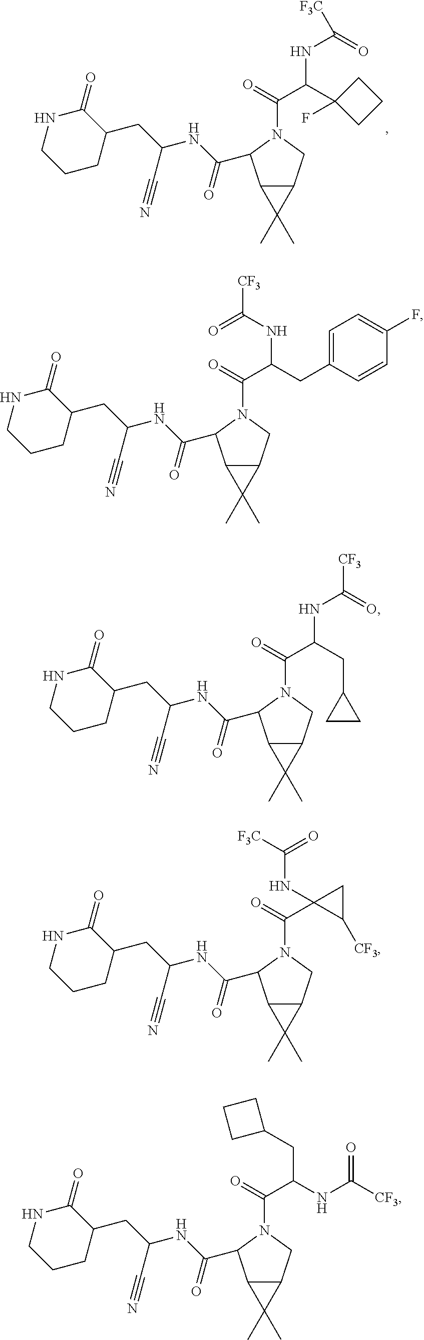

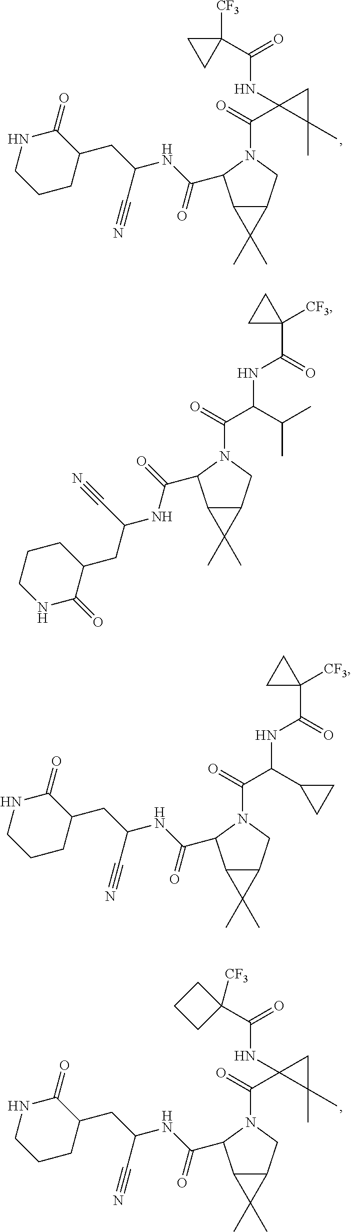

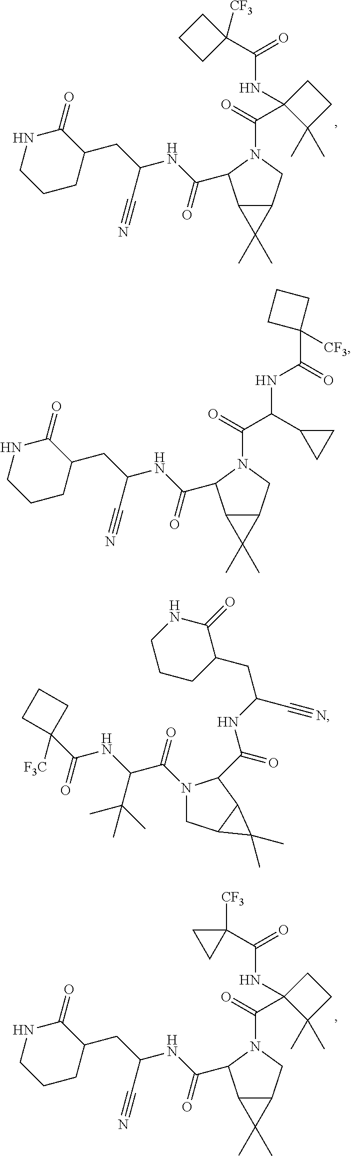

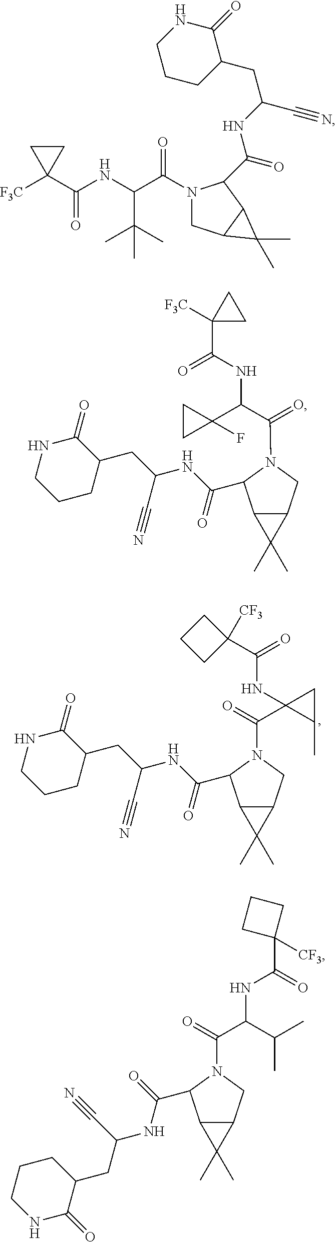

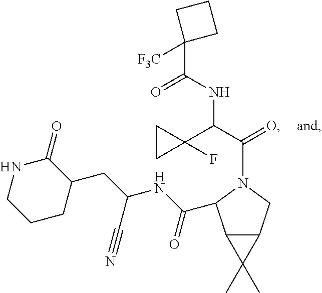 [SMILES: CC1(C)C2CN(C(=O)C(NC(=O)C3(C(F)(F)F)CCC3)C3(F)CC3)C(C(=O)NC(C#N)CC3CCCNC3=O)C21]

or a pharmaceutically acceptable salt thereof.

Procedures for making compounds described herein are provided in the examples below. In the reactions described below, it may be necessary to protect reactive functional groups (such as hydroxyl, amino, thio or carboxyl groups) to avoid their unwanted participation in the reactions. The incorporation of such groups, and the methods required to introduce and remove them are known to those skilled in the art (for example, see Greene, Wuts, Protective Groups in Organic Synthesis. 2nd Ed (1999)). The deprotection step may be the final step in the synthesis such that the removal of protecting groups affords compounds of Formula I or II as disclosed herein. Starting materials used in the following scheme can be purchased or prepared by methods described in the chemical literature, or by adaptations thereof, using methods known by those skilled in the art. The order in which the steps are performed can vary depending on the groups introduced and the reagents used, but would be apparent to those skilled in the art.

Compounds of any of Formula I or II depicted below, or any of the intermediates described in the schemes above, can be further derivatised by using one or more standard synthetic methods known to those skilled in the art. Such methods can involve substitution, oxidation or reduction reactions. These methods can also be used to obtain or modify compounds of Formula I or II or any preceding intermediates by modifying, introducing or removing appropriate functional groups.

Where it is desired to obtain a particular enantiomer of a compound of Formula I or II this may be produced from a corresponding mixture of enantiomers by employing any suitable conventional procedure for resolving enantiomers known to those skilled in the art. For example, diastereomeric derivatives (such as salts) can be produced by reaction of a mixture of enantiomers of a compound of Formula I or II (such a racemate) and an appropriate chiral compound (such as a chiral base). The diastereomers can then be separated by any conventional means such as crystallization or chromatography, and the desired enantiomer recovered (such as by treatment with an acid in the instance where the diastereomer is a salt). Alternatively, a racemic mixture of esters can be resolved by kinetic hydrolysis using a variety of biocatalysts (for example, see Patel Stereoselective Biocatalysts, Marcel Decker, New York 2000).

In another resolution process a racemate of compounds of Formula I or II can be separated using chiral High Performance Liquid Chromatography. Alternatively, a particular enantiomer can be obtained by using an appropriate chiral intermediate in one of the processes described above. Chromatography, recrystallisation and other conventional separation procedures may also be used with intermediates or final products where it is desired to obtain a particular geometric isomer of the disclosure.

Also provided herein is a broad spectrum, covalent 3CL or 3C protease antiviral compound, comprising a nitrile warhead covalently bound to a 3CL protease inhibitor, wherein the antiviral compound covalently binds to Cys on the protease, and wherein the antiviral compound is active against multiple viruses. In some embodiments, the broad spectrum covalent compound of Formula I, wherein the compound is active against caliciviruses, picornaviruses and coronaviruses. In some embodiments, the broad spectrum covalent compound of Formula I or II wherein the compound is active against caliciviruses, picornaviruses and coronaviruses. In some embodiments, the broad spectrum covalent compound of Formula II, wherein the compound is active against caliciviruses, picornaviruses and coronaviruses.

In some embodiments, a compound disclosed herein may act as monovalent protein degrader. For example, in some embodiments, a disclosed compound may act as a monovalent ligand that can induce the degradation of a protein of interest, for example, a 3C-like protease (e.g., 3CLpro, nsp5, or Mpro), by producing conformational or other changes that make the protein susceptible to detection by ubiquitin-degradation machinery.

### II. Methods

Another aspect of the disclosure provides methods of treating patients suffering from a viral infection, e.g., a coronaviral infection. In particular, in certain embodiments, the disclosure provides a method of treating the below medical indications comprising administering to a subject in need thereof a therapeutically effective amount of a compound described herein, such as a compound of Formula I, 11, 111, X, XI, or XII.

In certain embodiments, the disclosure provides a method of ameliorating or treating a viral infection in a patient in need thereof, comprising administering to the patient a therapeutically effective amount of any of the compounds described herein. In some embodiments, the viral infection is from a virus selected from the group consisting of an RNA virus, a DNA virus, a coronavirus, an astrovirus, a papillomavirus, a pneumovirus, a picomavirus, an influenza virus, an adenovirus, a cytomegalovirus, a polyomavirus, a poxvirus, a flavivirus, an alphavirus, an ebola virus, a morbillivirus, an enterovirus, an orthopneumovirus, a lentivirus, arenavirus, a herpes virus, and a hepatovirus. In certain embodiments, the viral infection is a coronavirus infection. In some embodiments, the viral infection is a coronavirus selected from the group consisting of: 229E alpha coronavirus, NL63 alpha coronavirus, OC43 beta coronavirus, HKU 1 beta coronavirus, Middle East Respiratory Syndrome (MERS) coronavirus (MERS-CoV), severe acute respiratory syndrome (SARS) coronavirus (SARS-CoV), and SARS-CoV-2 (COVID-19). In embodiments, the viral infection is SARS-CoV-2.

In some embodiments, the viral infection is from a virus selected from the group consisting of caliciviruses, MD 145, murine norovirus, vesicular exanthema of swine virus, abbit hemorrhagic disease virus, porcine teschovirus, bovine coronavirus, feline infectious peritonitis virus, EV-68 virus, EV-71 virus, poliovirus, norovirus, human rhinovirus (HRV), hepatitis A virus (HAV) and foot-and-mouth disease virus (FMDV).

In embodiments, the viral infection is an arenovirus infection. In some embodiments, the arenovirus is selected from the group consisting of: Junin virus, Lassa virus, Lujo virus, Machupo virus, and Sabia virus. In some embodiments, the viral infection is an influenza infection. In some embodiments, the influenza is influenza H1N1, H3N2 or H5N1.

Another aspect of the disclosure provides methods of treating patients suffering from a viral infection, e.g., a noroviral infection. In some embodiments, the disclosure provides a method of treating a viral infection from a norovirus in a patient in need thereof, comprising administering to the patient a therapeutically effective amount of any of the compounds described herein.

Also provided herein, in certain embodiments, is a method of inhibiting transmission of a virus, a method of inhibiting viral replication, a method of minimizing expression of viral proteins, or a method of inhibiting virus release, comprising administering a therapeutically effective amount of a compound described herein to a patient suffering from the virus, and/or contacting an effective amount of a compound described herein with a virally infected cell. In some embodiments, the method further comprises administering another therapeutic. In some embodiments, the method further comprises administering an additional anti-viral therapeutic. In embodiments, the anti-viral therapeutic is selected from the group consisting of ribavirin, favipiravir, ST-193, oseltamivir, zanamivir, peramivir, danoprevir, ritonavir, remdesivir, cobicistat, elvitegravir, emtricitabine, tenofovir, tenofovir disoproxil, tenofovir alafenamide hemifumarate, abacavir, dolutegravir, efavirenz, elbasvir, ledipasvir, glecaprevir, sofosbuvir, bictegravir, dasabuvir, lamivudine, atazanavir, ombitasvir, lamivudine, stavudine, nevirapine, rilpivirine, paritaprevir, simeprevir, daclatasvir, grazoprevir, pibrentasvir, adefovir, amprenavir, ampligen, aplaviroc, anti-caprine antibody, balavir, cabotegravir, cytarabine, ecoliever, epigallocatechin gallate, etravirine, fostemsavir, gemcitabine, griffithsin, imunovir, indinavir, maraviroc, methisazone, MK-2048, nelfmavir, nevirapine, nitazoxanide, norvir, plerixafor, PRO 140, raltegravir, pyramidine, saquinavir, telbivudine, TNX-355, valacyclovir, VIR-576, and zalcitabine. In some embodiments, the another therapeutic is selected from the group consisting of protease inhibitors, fusion inhibitors, M2 proton channel blockers, polymerase inhibitors, 6-endonuclease inhibitors, neuraminidase inhibitors, reverse transcriptase inhibitor, aciclovir, acyclovir, protease inhibitors, arbidol, atazanavir, atripla, boceprevir, cidofovir, combivir, darunavir, docosanol, edoxudine, entry inhibitors, entecavir, famciclovir, fomivirsen, fosamprenavir, foscarnet, fosfonet, ganciclovir, ibacitabine, immunovir, idoxuridine, imiquimod, inosine, integrase inhibitor, interferons, lopinavir, loviride, moroxydine, nexavir, nucleoside analogues, penciclovir, pleconaril, podophyllotoxin, ribavirin, tipranavir, trifluridine, trizivir, tromantadine, truvada, valaciclovir, valganciclovir, vicriviroc, vidarabine, viramidine, and zodovudine. In embodiments, the additional anti-viral therapeutic is selected from the group consisting of lamivudine, an interferon alpha, a VAP anti-idiotypic antibody, enfuvirtide, amantadine, rimantadine, pleconaril, aciclovir, zidovudine, fomivirsen, a morpholino, a protease inhibitor, double-stranded RNA activated caspase oligomerizer (DRACO), rifampicin, zanamivir, oseltamivir, danoprevir, ritonavir, remdesivir, cobicistat, elvitegravir, emtricitabine, tenofovir, tenofovir disoproxil, tenofovir alafenamide hemifumarate, abacavir, dolutegravir, efavirenz, elbasvir, ledipasvir, glecaprevir, sofosbuvir, bictegravir, dasabuvir, lamivudine, atazanavir, ombitasvir, lamivudine, stavudine, nevirapine, rilpivirine, paritaprevir, simeprevir, daclatasvir, grazoprevir, pibrentasvir, adefovir, amprenavir, ampligen, aplaviroc, anti-caprine antibody, balavir, cabotegravir, cytarabine, ecoliever, epigallocatechin gallate, etravirine, fostemsavir, gemcitabine, griffithsin, imunovir, indinavir, maraviroc, methisazone, MK-2048, nelfmavir, nevirapine, nitazoxanide, norvir, plerixafor, PRO 140, raltegravir, pyramidine, saquinavir, telbivudine, TNX-355, valacyclovir, VIR-576, and zalcitabine.

Contemplated patients include not only humans, but other animals such as companion animals (e.g. dogs, cats), domestic animals (e.g. cow, swine), and wild animals (e.g. monkeys, bats, snakes).

Accordingly, in one embodiment, described herein is a method of ameliorating or treating a viral infection in a patient in need thereof, comprising administering to the patient a therapeutically effective amount of a compound described herein (e.g., a compound of Formula I, II, III, X, XI, or XII described herein) or a pharmaceutically acceptable salt thereof.

Other contemplated methods of treatment include method of treating or ameliorating a virus infection condition or co-morbidity, by administering a compound disclosed herein to a subject.

Exemplary co-morbidities include lung diseases, cardiac disorders, endocrine disorders, respiratory disorders, hepatic disorders, skeletal disorders, psychiatric disorders, metabolic disorders, and reproductive disorders.

In some embodiments, the viral infection is from a virus selected from the group consisting of an RNA virus, a DNA virus, a coronavirus, a papillomavirus, a pneumovirus, a picornavirus, an influenza virus, an adenovirus, a cytomegalovirus, a polyomavirus, a poxvirus, a flavivirus, an alphavirus, an ebola virus, a morbillivirus, an enterovirus, an orthopneumovirus, a lentivirus, arenovirus, a herpes virus, and a hepatovirus. In some embodiments, the viral infection is a coronavirus infection. In some embodiments, the viral infection is a coronavirus selected from the group consisting of: 229E alpha coronavirus, NL63 alpha coronavirus, OC43 beta coronavirus, HKU 1 beta coronavirus, Middle East Respiratory Syndrome (MERS) coronavirus (MERS-CoV), severe acute respiratory syndrome (SARS) coronavirus (SARS-CoV), and SARS-CoV-2 (COVID-19). In some embodiments, the viral infection is SARS-CoV-2. In some embodiments, the viral infection is an arenovirus infection. In some embodiments, the arenovirus is selected from the group consisting of: Junin virus, Lassa virus, Lujo virus, Machupo virus, and Sabia virus. In some embodiments, the viral infection is an influenza infection. In some embodiments, the influenza is influenza H1 N1, H3N2 or H5N1. In some embodiments, the viral infection is a respiratory viral infection. In some embodiments, the viral infection is an upper respiratory viral infection or a lower respiratory viral infection. In some embodiments, the method further comprises administering another therapeutic.

In certain embodiments, the virus is selected from the group consisting of a retrovirus (e.g., human immunodeficiency virus (HIV), simian immunodeficiency virus (SW), human T-cell lymphotropic virus (HTLV)-1, HTLV-2, HTLV-3, HTLV-4), Ebola virus, hepatitis A virus, hepatitis B virus, hepatitis C virus, a herpes simplex virus (HSV) (e.g., HSV-1, HSV-2, varicella zoster virus, cytomegalovirus), an adenovirus, an orthomyxovirus (e.g., influenza virus A, influenza virus B, influenza virus C, influenza virus D, thogotovirus), a flavivirus (e.g., dengue virus, Zika virus), West Nile virus, Rift Valley fever virus, an arenavirus, Crimean-Congo hemorrhagic fever virus, an echovirus, a rhinovirus, coxsackie virus, a coronavirus (e.g., Severe acute respiratory syndrome coronavirus 2 (SARS-CoV-2), coronavirus disease 2019 (COVID-19), a respiratory syncytial virus, a mumps virus, a rotavirus, measles virus, rubella virus, a parvovirus (e.g., an adeno-associated virus), a vaccinia virus, a variola virus, a molluscum virus, bovine leukemia virus, bovine diarrhea virus, a poliovirus, St. Louis encephalitis virus, Japanese encephalitis virus, a tick-borne encephalitis virus, Murray Valley virus, Powassan virus, Rocio virus, louping-ill virus, Banzi virus, llheus virus, Kokobera virus, Kunjin virus, Alfuy virus, a rabies virus, a polyomavirus (e.g., JC virus, BK virus), an alphavirus, and a rubivirus (e.g., rubella virus).

In certain embodiments, the disease or disorder is a viral infection, e.g., a disease or disorder selected from the group consisting of acquired immune deficiency syndrome (AIDS), HTLV-1 associated myelopathy/tropical spastic paraparesis, Ebola virus disease, hepatitis A, hepatitis B, hepatitis C, herpes, herpes zoster, acute varicella, mononucleosis, respiratory infections, pneumonia, influenza, dengue fever, encephalitis (e.g., Japanese encephalitis, St. Louis encephalitis, or tick-borne encephalitis such as Powassan encephalitis), West Nile fever, Rift Valley fever, Crimean-Congo hemorrhagic fever, Kyasanur Forest disease, Yellow fever, Zika fever, aseptic meningitis, myocarditis, common cold, lung infections, molloscum contagiosum, enzootic bovine leucosis, coronavirus disease 2019 (COVID-19), mumps, gastroenteritis, measles, rubella, slapped-cheek disease, smallpox, warts (e.g., genital warts), molluscum contagiosum, polio, rabies, and pityriasis rosea.

In some embodiments, the virus is an RNA virus (having a genome that is composed of RNA). RNA viruses may be single-stranded RNA (ssRNA) or double-stranded RNA (dsRNA). RNA viruses have high mutation rates compared to DNA viruses, as RNA polymerase lacks proofreading capability (see Steinhauer DA, Holland JJ (1987). “Rapid evolution of RNA viruses”. Annu. Rev. Microbiol. 41: 409-33). In some embodiments, the RNA virus is a positive-strand RNA virus (e.g., a SARS-CoV virus, polio virus, Coxsackie virus, Enterovirus, Human rhinovirus, Foot/Mouth disease virus, encephalomyocarditis virus, Dengue virus, Zika virus, Hepatitis C virus, or New Castle Disease virus).

RNA viruses are classified by the type of genome (double-stranded, negative (−), or positive (+) single-stranded). Double-stranded RNA viruses contain a number of different RNA molecules, each coding for one or more viral proteins. Positive-sense ssRNA viruses utilize their genome directly as mRNA; ribosomes within the host cell translate mRNA into a single protein that is then modified to form the various proteins needed for viral replication. One such protein is RNA-dependent RNA polymerase (RNA replicase), which copies the viral RNA in order to form a double-stranded, replicative form. Negative-sense ssRNA viruses have their genome copied by an RNA replicase enzyme to produce positive-sense RNA for replication. Therefore, the virus comprises an RNA replicase enzyme. The resultant positive-sense RNA then acts as viral mRNA and is translated by the host ribosomes. In some embodiments, the virus is a dsRNA virus. In some embodiments, the virus is a negative ssRNA virus. In some embodiments, the virus is a positive ssRNA virus. In some embodiments, the positive ssRNA virus is a coronavirus.

SARS-CoV2, also sometimes referred to as the novel coronavirus of 2019 or 2019-nCoV, is a positive-sense single-stranded RNA virus. SARS-CoV-2 has four structural proteins, known as the S (spike), E (envelope), M (membrane), and N (nucleocapsid) proteins. The N protein holds the RNA genome together; the S, E, and M proteins form the viral envelope. Spike allows the virus to attach to the membrane of a host cell, such as the ACE2 receptor in human cells (Kruse R.L. (2020), Therapeutic strategies in an outbreak scenario to treat the novel coronavirus originating in Wuhan, China (version 2). F1000Research, 9:72). SARS-CoV2 is the highly contagious, causative viral agent of coronavirus disease 2019 (COVID 19), a global pandemic.

In some embodiments, the virus is a DNA virus (having a genome that is composed of DNA). Exemplary DNA viruses include, without limitation, parvoviruses (e.g., adeno-associated viruses), adenoviruses, asfarviruses, herpesviruses (e.g., herpes simplex virus 1 and 2 (HSV-1 and HSV-2), Epstein-Barr virus (EBV), cytomegalovirus (CMV)), papillomoviruses (e.g., HPV), polyomaviruses (e.g., simian vacuolating virus 40 (SV40)), and poxviruses (e.g., vaccinia virus, cowpox virus, smallpox virus, fowlpox virus, sheeppox virus, myxoma virus). Exemplary RNA viruses include, without limitation, bunyaviruses (e.g., hantavirus), coronaviruses, flaviviruses (e.g., yellow fever virus, west nile virus, dengue virus), hepatitis viruses (e.g., hepatitis A virus, hepatitis C virus, hepatitis E virus), influenza viruses (e.g., influenza virus type A, influenza virus type B, influenza virus type C), measles virus, mumps virus, noroviruses (e.g., Norwalk virus), poliovirus, respiratory syncytial virus (RSV), retroviruses (e.g., human immunodeficiency virus-1 (HIV-1)) and toroviruses.

The methods described herein may inhibit viral replication transmission, replication, assembly, or release, or minimize expression of viral proteins. In one embodiment, described herein is a method of inhibiting transmission of a virus, a method of inhibiting viral replication, a method of minimizing expression of viral proteins, or a method of inhibiting virus release, comprising administering a therapeutically effective amount of a compound described herein, or a pharmaceutically acceptable salt thereof, to a patient suffering from the virus, and/or contacting an effective amount of a compound described herein or a pharmaceutically acceptable salt thereof, with a virally infected cell.

Also described herein is a method of treating a respiratory disorder in a subject in need thereof, comprising administering to the patient a therapeutically effective amount of a compound described herein (e.g., a compound of Formula I, II, III, X, XI, or XII described herein) or a pharmaceutically acceptable salt thereof. In embodiments, the respiratory disorder is selected from the group consisting of chronic obstructive pulmonary disease (COPD), asthma, fibrosis, chronic asthma, acute asthma, lung disease secondary to environmental exposures, acute lung infection, chronic lung infection, al antitrypsin disease, cystic fibrosis and an autoimmune disease. In some embodiments, the respiratory disorder is associated with a heart attack.

Also described herein is a method of treating a disorder associated with cathepsin (e.g. Cathepsin K) in a subject in need thereof, comprising administering to the patient a therapeutically effective amount of a compound described herein (e.g., a compound of Formula I, II, III, X, XI, or XII described herein) or a pharmaceutically acceptable salt thereof. In some embodiments, the disorder is a cathepsin dependent condition or disease. In embodiments, the disorder is selected from the group consisting of breast cancer, pycnodysostosis, glioblastoma, osteosclerosis, osteoporosis, glucocorticoid induced osteoporosis, Paget's disease, abnormally increased bone turnover, periodontal disease, tooth loss, bone fractures, rheumatoid arthritis, osteoarthritis, periprosthetic osteolysis, osteogenesis imperfecta, atherosclerosis, obesity, glaucoma, chronic obstructive pulmonary disease, metastatic bone disease, hypercalcemia of malignancy, and multiple myeloma.

Compounds described herein, e.g., a compound of Formula I, II, III, X, XI, or XII as defined herein, can be administered in combination with one or more additional therapeutic agents to treat a disorder described herein, such as an infection by a pathogen described herein, e.g., a virus, fungus, or protozoan. For clarity, contemplated herein are both a fixed composition comprising a disclosed compound and another therapeutic agent such as disclosed herein, and methods of administering, separately a disclosed compound and a disclosed therapeutic. For example, provided in the present disclosure is a pharmaceutical composition comprising a compound described herein, e.g., a compound of Formula I or II as defined herein, one or more additional therapeutic agents, and a pharmaceutically acceptable excipient. In some embodiments, a compound of Formula I or II as defined herein and one additional therapeutic agent is administered. In some embodiments, a disclosed compound as defined herein and two additional therapeutic agents are administered. In some embodiments, a disclosed compound as defined herein and three additional therapeutic agents are administered. Combination therapy can be achieved by administering two or more therapeutic agents, each of which is formulated and administered separately. For example, a compound of Formula I or II as defined herein and an additional therapeutic agent can be formulated and administered separately. Combination therapy can also be achieved by administering two or more therapeutic agents in a single formulation, for example a pharmaceutical composition comprising a compound of Formula I as one therapeutic agent and one or more additional therapeutic agents such as an antibiotic, a viral protease inhibitor, or an anti-viral nucleoside anti-metabolite. For example, a compound of Formula I as defined herein and an additional therapeutic agent can be administered in a single formulation. Other combinations are also encompassed by combination therapy. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or weeks of each other. In some cases even longer intervals are possible. While in many cases it is desirable that the two or more agents used in a combination therapy be present in within the patient's body at the same time, this need not be so.

Combination therapy can also include two or more administrations of one or more of the agents used in the combination using different sequencing of the component agents. For example, if agent X and agent Y are used in a combination, one could administer them sequentially in any combination one or more times, e.g., in the order X—Y—X, X—X—Y, Y—X—Y, Y—Y—X, X—X—Y—Y, etc.

In some embodiments, the one or more additional therapeutic agents that may be administered in combination with a compound provided herein can be an antibiotic, a viral protease inhibitor, an anti-viral anti-metabolite, a lysosomotropic agent, a M2 proton channel blocker, a polymerase inhibitor (e.g., EIDD-2801, which is also known as MOLNUPIRAVIR), a neuraminidase inhibitor, a reverse transcriptase inhibitor, a viral entry inhibitor, an integrase inhibitor, interferons (e.g., types I, II, and III), or a nucleoside analogue. In some embodiments, the one or more additional therapeutic agents that may be administered in combination wiht a compounds provided herein can be a steroid (e.g., corticosteroids, such as bethamethasone, prednisone, prednisolone, triamcinolone, methylprednisolone, dexamethasone; mineralcorticoid such as fludrocortisone; glucocorticoids, such as hydrocortisone, cortisone, ethamethasoneb, prednisone, prednisolone, triamcinolone, dexamethasone; vitamin D such as dihydrotachysterol; androgens such as apoptone, oxandrolone, oxabolone, testosterone, nandrolone (also known as anabolic steroids), oestrogens such as diethylstilbestrol, progestins such as danazol, norethindrone, medroxyprogesterone acetate, 17-Hydroxyprogesterone caproate; and progestins such as mifepristone and gestrinone) or an immunomodulator (e.g., 6-Mercaptopurine, 6MP, Alferon N, anakinra, Arcalyst, Avonex, AVOSTARTGRIP, Bafiertam, Berinert, Betaseron, BG-12, C1 esterase inhibitor recombinant, C1 inhibitor human, Cinryze, Copaxone, dimethyl fumarate, diroximel fumarate, ecallantide, emapalumab, emapalumab-lzsg, Extavia, fingolimod, Firazyr, Gamifant, Gilenya, glatiramer, Glatopa, Haegarda, icatibant, Infergen, interferon alfa n3, interferon alfacon 1, interferon beta 1a, interferon beta 1b, Kalbitor, Kineret, mercaptopurine, monomethyl fumarate, peginterferon beta-1a, Plegridy, Purinethol, Purixan, Rebif, Rebif Rebidose, remestemcel-L, rilonacept, ropeginterferon alfa 2b, Ruconest, Ryoncil, siltuximab, sutimlimab, Sylvant, Tecfidera, and Vumerity). In some embodiments, the one or more additional therapeutic agent is Cathepsin L. In some embodiments, the one or more additional therapeutic agent is dehydrodidemnin B (also known as Plitidepsin or APLIDIN) or Zotatifin (eFT226).

In some embodiments, methods described herein further comprise administering an additional anti-viral therapeutic. In some embodiments, the anti-viral therapeutic is selected from the group consisting of ribavirin, favipiravir, ST-193, oseltamivir, zanamivir, peramivir, danoprevir, ritonavir, remdesivir, cobicistat, elvitegravir, emtricitabine, tenofovir, tenofovir disoproxil, tenofovir alafenamide hemifumarate, abacavir, dolutegravir, efavirenz, elbasvir, ledipasvir, glecaprevir, sofosbuvir, bictegravir, dasabuvir, lamivudine, atazanavir, ombitasvir, lamivudine, stavudine, nevirapine, rilpivirine, paritaprevir, simeprevir, daclatasvir, grazoprevir, pibrentasvir, adefovir, amprenavir, ampligen, aplaviroc, anti-caprine antibody, balavir, cabotegravir, cytarabine, ecoliever, epigallocatechin gallate, etravirine, fostemsavir, gemcitabine, griffithsin, imunovir, indinavir, maraviroc, methisazone, MK-2048, nelfmavir, nevirapine, nitazoxanide, norvir, plerixafor, PRO 140, raltegravir, pyramidine, saquinavir, telbivudine, TNX-355, valacyclovir, VIR-576, and zalcitabine. In some embodiments, the another therapeutic is selected from the group consisting of protease inhibitors (e.g., nafamostat, camostat, gabexate, epsilon-aminocapronic acid and aprotinin), fusion inhibitors (e.g., BMY-27709, CL 61917, and CL 62554), M2 proton channel blockers (e.g., amantadine and rimantadine), polymerase inhibitors (e.g., 2-deoxy-2′fluoroguanosides (2′-fluoroGuo), 6-endonuclease inhibitors (e.g., L-735,822 and flutamide) neuraminidase inhibitors (e.g., zanamivir (Relenza), oseltamivir, peramivir and ABT-675 (A-315675), reverse transcriptase inhibitor (e.g., abacavir, adefovir, delavirdine, didanosine, efavirenz, emtricitabine, lamivudine, nevirapine, stavudine, tenofovir, tenofovir disoproxil, and zalcitabine), acyclovir, acyclovir, protease inhibitors (e.g., amprenavir, indinavir, nelfinavir, ritonavir, and saquinavir), arbidol, atazanavir, atripla, boceprevir, cidofovir, combivir, darunavir, docosanol, edoxudine, entry inhibitors (e.g., enfuvirtide and maraviroc), entecavir, famciclovir, fomivirsen, fosamprenavir, foscarnet, fosfonet, ganciclovir, ibacitabine, immunovir, idoxuridine, imiquimod, inosine, integrase inhibitor (e.g., raltegravir), interferons (e.g., types I, II, and III), lopinavir, loviride, moroxydine, nexavir, nucleoside analogues (e.g., aciclovir), penciclovir, pleconaril, podophyllotoxin, ribavirin, tipranavir, trifluridine, trizivir, tromantadine, truvada, valaciclovir, valganciclovir, vicriviroc, vidarabine, viramidine, and zodovudine. In some embodiments, the additional anti-viral therapeutic is selected from the group consisting of lamivudine, an interferon alpha, a VAP anti-idiotypic antibody, enfuvirtide, amantadine, rimantadine, pleconaril, aciclovir, zidovudine, fomivi sen, a morpholino, a protease inhibitor, double-stranded RNA activated caspase oligomerizer (DRACO), rifampicin, zanamivir, oseltamivir, danoprevir, ritonavir, remdesivir, cobicistat, elvitegravir, emtricitabine, tenofovir, tenofovir disoproxil, tenofovir alafenamide hemifumarate, abacavir, dolutegravir, efavirenz, elbasvir, ledipasvir, glecaprevir, sofosbuvir, bictegravir, dasabuvir, lamivudine, atazanavir, ombitasvir, lamivudine, stavudine, nevirapine, rilpivirine, paritaprevir, simeprevir, daclatasvir, grazoprevir, pibrentasvir, adefovir, amprenavir, ampligen, aplaviroc, anti-caprine antibody, balavir, cabotegravir, cytarabine, ecoliever, epigallocatechin gallate, etravirine, fostemsavir, gemcitabine, griffithsin, imunovir, indinavir, maraviroc, methisazone, MK-2048, nelfmavir, nevirapine, nitazoxanide, norvir, plerixafor, PRO 140, raltegravir, pyramidine, saquinavir, telbivudine, TNX-355, valacyclovir, VIR-576, and zalcitabine. In some embodiments, the another therapeutic is selected from the group consisting of quinine (optionally in combination with clindamycin), chloroquine, amodiaquine, artemisinin and its derivatives (e.g., artemether, artesunate, dihydroartemisinin, arteether), doxycycline, pyrimethamine, mefloquine, halofantrine, hydroxychloroquine, eflornithine, nitazoxanide, ornidazole, paromomycin, pentamidine, primaquine, pyrimethamine, proguanil (optionally in combination with atovaquone), a sulfonamide (e.g., sulfadoxine, sulfamethoxypyridazine), tafenoquine, tinidazole and a PPT1 inhibitor (including Lys05 and DC661). In some embodiments, the another therapeutic is an antibiotic. In some embodiments, the antibiotic is a penicillin antibiotic, a quinolone antibiotic, a tetracycline antibiotic, a macrolide antibiotic, a lincosamide antibiotic, a cephalosporin antibiotic, or an RNA synthetase inhibitor. In some embodiments, the antibiotic is selected from the group consisting of azithromycin, vancomycin, metronidazole, gentamicin, colistin, fidaxomicin, telavancin, oritavancin, dalbavancin, daptomycin, cephalexin, cefuroxime, cefadroxil, cefazolin, cephalothin, cefaclor, cefamandole, cefoxitin, cefprozil, ceftobiprole, cipro, Levaquin, floxin, tequin, avelox, norflox, tetracycline, minocycline, oxytetracycline, doxycycline, amoxicillin, ampicillin, penicillin V, dicloxacillin, carbenicillin, methicillin, ertapenem, doripenem, imipenem/cilastatin, meropenem, amikacin, kanamycin, neomycin, netilmicin, tobramycin, paromomycin, cefixime, cefdinir, cefditoren, cefoperazone, cefotaxime, ceftazidime, ceftibuten, ceftizoxime, ceftriaxone, cefoxotin, and streptomycin. In some embodiments, the antibiotic is azithromycin.

In some embodiments, the one or more additional therapeutic agents that may be administered in combination with a compound provided herein can be selected from the group consisting of ribavirin, favipiravir, ST-193, oseltamivir, zanamivir, peramivir, danoprevir, ritonavir, remdesivir, cobicistat, elvitegravir, emtricitabine, tenofovir, tenofovir disoproxil, tenofovir alafenamide hemifumarate, abacavir, dolutegravir, efavirenz, elbasvir, ledipasvir, glecaprevir, sofosbuvir, bictegravir, dasabuvir, lamivudine, atazanavir, ombitasvir, lamivudine, stavudine, nevirapine, rilpivirine, paritaprevir, simeprevir, daclatasvir, grazoprevir, pibrentasvir, adefovir, amprenavir, ampligen, aplaviroc, anti-caprine antibody, balavir, cabotegravir, cytarabine, ecoliever, epigallocatechin gallate, etravirine, fostemsavir, gemcitabine, griffithsin, imunovir, indinavir, maraviroc, methisazone, MK-2048, nelfmavir, nevirapine, nitazoxanide, norvir, plerixafor, PRO 140, raltegravir, pyramidine, saquinavir, telbivudine, TNX-355, valacyclovir, VIR-576, and zalcitabine.

In some embodiments, the compounds described herein (e.g. a compound of Formula I or II) and pharmaceutically acceptable salts thereof may be used in combination with one or more other agents which may be useful in the prevention or treatment of respiratory disease, inflammatory disease, autoimmune disease, for example; anti-histamines, corticosteroids, (e.g., fluticasone propionate, fluticasone furoate, beclomethasone dipropionate, budesonide, ciclesonide, mometasone furoate, triamcinolone, flunisolide), NSAIDs, leukotriene modulators (e.g., montelukast, zafirlukast.pranlukast), tryptase inhibitors, IKK2 inhibitors, p38 inhibitors, Syk inhibitors, protease inhibitors such as elastase inhibitors, integrin antagonists (e.g., beta-2 integrin antagonists), adenosine A2a agonists, mediator release inhibitors such as sodium chromoglycate, 5-lipoxygenase inhibitors (zyflo), DP1 antagonists, DP2 antagonists, P13K delta inhibitors, ITK inhibitors, LP (lysophosphatidic) inhibitors or FLAP (5-lipoxygenase activating protein) inhibitors (e.g., sodium 3-(3-(tert-butylthio)-1-(4-(6-ethoxypyridin-3-yl)benzyl)-5-((5-ethylpyridin-2-yl)methoxy)-1 H-indol-2-yl)-2,2-dimethylpropanoate), bronchodilators (e.g., muscarinic antagonists, beta-2 agonists), methotrexate, and similar agents; monoclonal antibody therapy such as anti-1gE, anti-TNF, anti-IL-S, anti-IL-6, anti-IL-12, anti-IL-1 and similar agents; cytokine receptor therapies e.g. etanercept and similar agents; antigen non-specific immunotherapies (e.g. interferon or other cytokines/chemokines, chemokine receptor modulators such as CCR3, CCR4 or CXCR2 antagonists, other cytokine/chemokine agonists or antagonists, TLR agonists and similar agents), suitable anti-infective agents including antibiotic agents, antifungal agents, anthelmintic agents, antimalarial agents, antiprotozoal agents and antituberculosis agents.

In some embodiments, the additional therapeutic agents can be kinase inhibitors including but not limited to erlotinib, gefitinib, neratinib, afatinib, osimertinib, lapatanib, crizotinib, brigatinib, ceritinib, alectinib, lorlatinib, everolimus, temsirolimus, abemaciclib, LEE011, palbociclib, cabozantinib, sunitinib, pazopanib, sorafenib, regorafenib, sunitinib, axitinib, dasatinib, imatinib, nilotinib, ponatinib, idelalisib, ibrutinib, Loxo 292, larotrectinib, and quizartinib.

In some embodiments, the additional therapeutic agents can be therapeutic anti viral vaccines.

In some embodiments, the additional therapeutic agents can be immunomodulatory agents including but not limited to anti-PD-lor anti-PDL-1 therapeutics including pembrolizumab, nivolumab, atezolizumab, durvalumab, BMS-936559, or avelumab, anti-TIM3 (anti-HAVcr2) therapeutics including but not limited to TSR-022 or MBG453, anti-LAG3 therapeutics including but not limited to relatlimab, LAG525, or TSR-033, anti-4-1BB (anti-CD37, anti-TNFRSF9), CD40 agonist therapeutics including but not limited to SGN-40, CP-870,893 or R07009789, anti-CD47 therapeutics including but not limited to Hu5F9-G4, anti-CD20 therapeutics, anti-CD38 therapeutics, STING agonists including but not limited to ADU-S 100, MK-1454, ASA404, or amidobenzimidazoles, anthracyclines including but not limited to doxorubicin or mitoxanthrone, hypomethylating agents including but not limited to azacytidine or decitabine, other immunomodulatory therapeutics including but not limited to epidermal growth factor inhibitors, statins, metformin, angiotensin receptor blockers, thalidomide, lenalidomide, pomalidomide, prednisone, or dexamethasone. In some embodiments, the additional therapeutic agent is a p2-adrenoreceptor agonist including, but not limited to, vilanterol, salmeterol, salbutamol.formoterol, salmefamol, fenoterol carmoterol, etanterol, naminterol, clenbuterol, pirbuterol.flerbuterol, reproterol, bambuterol, indacaterol, terbutaline and salts thereof, for example the xinafoate (1-hydroxy-2-naphthalenecarboxylate) salt of salmeterol, the sulphate salt of salbutamol or the fumarate salt of formoterol. In some embodiments, the additional therapeutic agent is an anticholinergic agent, including, but not limited to, umeclidinium (for example, as the bromide), ipratropium (for example, as the bromide), oxitropium (for example, as the bromide) and tiotropium (for example, as the bromide).

In particular, in certain embodiments, the disclosure provides a method of treating the above medical indications comprising administering a subject in need thereof a therapeutically effective amount of a compound described herein, such as a disclosed compound.

The term “boosting amount” or “boosting dose” is the amount of a compound needed to improve the pharmacokinetics of a second compound (or increase availability or exposure). The boosting amount or boosting dose may improve the pharmacokinetics (or increase availability or exposure) of the second compound to a level to therapeutic levels in a subject.

In one embodiment, the disclosure provides for a disclosed compound to be administered together with an antiviral therapeutic such as disclosed herein, and e.g., thereby boosting the dose of the anti-viral therapeutic or therapeutics. Such a boost combination may be used, e.g., as prophylactic or therapeutic treatment of a viral infection in a subject in need thereof. In one embodiment, the protease inhibitor is a compound described herein (e.g. a compound of Formula I, II, III, X, XI, or XII).

### III. Pharmaceutical Compositions and Kits

Another aspect of the disclosure provides pharmaceutical compositions comprising compounds as disclosed herein formulated together with a pharmaceutically acceptable carrier. In particular, the present disclosure provides pharmaceutical compositions comprising compounds as disclosed herein formulated together with one or more pharmaceutically acceptable carriers. These formulations include those suitable for oral, rectal, topical, buccal, parenteral (e.g., subcutaneous, intramuscular, intradermal, or intravenous) rectal, vaginal, or aerosol administration, although the most suitable form of administration in any given case will depend on the degree and severity of the condition being treated and on the nature of the particular compound being used. For example, disclosed compositions may be formulated as a unit dose, and/or may be formulated for oral or subcutaneous administration.

Exemplary pharmaceutical compositions of this disclosure may be used in the form of a pharmaceutical preparation, for example, in solid, semisolid or liquid form, which contains one or more of the compound of the disclosure, as an active ingredient, in admixture with an organic or inorganic carrier or excipient suitable for external, enteral or parenteral applications. The active ingredient may be compounded, for example, with the usual non toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets, the principal active ingredient may be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the disclosure, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent.

Dosage forms for transdermal administration of a subject composition include powders, sprays, ointments, pastes, creams, lotions, gels, solutions, patches and inhalants. The active component may be mixed under sterile conditions with a pharmaceutically acceptable carrier, and with any preservatives, buffers, or propellants which may be required

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions and compounds of the present disclosure may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions of this disclosure suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants

In another aspect, the disclosure provides enteral pharmaceutical formulations including a disclosed compound and an enteric material; and a pharmaceutically acceptable carrier or excipient thereof. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5. Accordingly, enteric materials are not soluble, for example, until a pH of about 5.0, of about 5.2, of about 5.4, of about 5.6, of about 5.8, of about 6.0, of about 6.2, of about 6.4, of about 6.6, of about 6.8, of about 7.0, of about 7.2, of about 7.4, of about 7.6, of about 7.8, of about 8.0, of about 8.2, of about 8.4, of about 8.6, of about 8.8, of about 9.0, of about 9.2, of about 9.4, of about 9.6, of about 9.8, or of about 10.0. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, natural resins such as zein, shellac and copal collophorium, and several commercially available enteric dispersion systems (e.g.; Eudragit L30D55, Eudragit FS30D, Eudragit L100, Eudragit S100, Kollicoat EMM30D, Estacryl 30D, Coateric, and Aquateric). The solubility of each of the above materials is either known or is readily determinable in vitro. The foregoing is a list of possible materials, but one of skill in the art with the benefit of the disclosure would recognize that it is not comprehensive and that there are other enteric materials that would meet the objectives of the present disclosure.

Advantageously, the disclosure also provides kits for use by a e.g. a consumer in need of 3CL inhibitor. Such kits include a suitable dosage form such as those described above and instructions describing the method of using such dosage form to mediate, reduce or prevent inflammation. The instructions would direct the consumer or medical personnel to administer the dosage form according to administration modes known to those skilled in the art. Such kits could advantageously be packaged and sold in single or multiple kit units. An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. . . . Second Week, Monday, Tuesday, . . . ” etc. Other variations of memory aids will be readily apparent. A “daily dose” can be a single tablet or capsule or several pills or capsules to be taken on a given day. Also, a daily dose of a first compound can consist of one tablet or capsule while a daily dose of the second compound can consist of several tablets or capsules and vice versa. The memory aid should reflect this.

Also contemplated herein are methods and compositions that include a second active agent, or administering a second active agent. For example, in addition to having a viral infection, a subject or patient can further have viral infection- or virus-related co-morbidities, i.e., diseases and other adverse health conditions associated with, exacerbated by, or precipitated by being infected by a virus. Contemplated herein are disclosed compounds in combination with at least one other agent that has previously been shown to treat these virus-related conditions.

### IV. Conjugates

In some embodiments, provide herein are conjugates represented by:

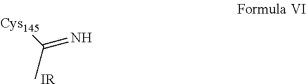

wherein Cys₁₄₅ is cysteine at position 145 or equivalent active site cysteine on a CL protease; and IR is a viral protease inhibitor.

### EXAMPLES

The compounds described herein can be prepared in a number of ways based on the teachings contained herein and synthetic procedures known in the art. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials.

The compounds described herein can be synthesized using methods disclosed in Tanaka, Y.; Hasui, T.; Suginome, M. Organic Letters 2007 9(22), 4407-4410, and Pan, S. C.; List, B. Angew. Chem. Int. Ed 2008, 47, 3622-3625, which are incorporated herein by reference.

At least some of the compounds identified as “Intermediates” herein are contemplated as compounds of the disclosure.

¹H NMR spectra are recorded at ambient temperature using e.g., a Varian Unity Inova (400 MHz) spectrometer with a triple resonance 5 mm probe for Example compounds, and either a Bruker Avance DRX (400 MHz) spectrometer or a Bruker Avance DPX (300 MHz) spectrometer for Intermediate compounds. Chemical shifts are expressed in ppm relative to tetramethylsilane. The following abbreviations have been used: br=broad signal, s =singlet, d=doublet, dd=double doublet, dt=double triplet, ddd=double double doublet, t =triplet, td=triple doublet, tdd=triple double doublet, q=quartet, m=multiplet.

Abbreviations:

| | |
|---|---|
| AcOH | acetic acid |
| ACN | acetonitrile |
| Boc | tert- butoxycarbonyl |
| | protecting group |
| Boc₂O | di-tert-butyl dicarbonate |
| BF₃•Et₂O | boron trifluoride diethyl |
| | etherate |
| CbzCl | benzyl chloroformate |
| Cbz-Osu | N- |
| | (benzyloxycarbon- |
| | yloxy)succinimide |
| CDI | 1,1′-carbonyldiimidazole |
| Cu(Oac)₂ | copper (II) acetate |
| DCE | dichloroethane |
| DCM | dichloromethane |
| DDQ | 2,3-dichloro-5,6-dicyano- |
| | 1,4-benzoquinone |
| DIEA | N,N-diisopropylethylamine |
| DIPEA | N,N-diisopropylethylamine |
| DMA | dimethylacetamide |
| DMAP | 4-dimethylaminopyridine |
| DMF | dimethylformamide |
| EA | ethyl acetate |
| EtOAc | ethyl acetate |
| EDCI | 1-ethyl-3- (3- |
| | dimethylaminopro- |
| | pyl)carbodiimide |
| EDTA | ethylenediaminetetraacetic |
| | acid |
| EtOH | ethanol |
| FA | formic acid |
| HATU | (1- |
| | [bis(dimethylamino)methyl- |
| | ene]-1H-1,2,3-triazolo[4,5- |
| | b]pyridinium 3-oxide |
| | hexafluorophosphate |
| HOBt | hydroxybenzotirazole |
| IPA | isopropanol |
| LiHMDS | lithium |
| | bis(trimethylsilyl)amide |
| MeI | methyl iodide |
| MTBA | 1-4-(3- |
| | methyltriazeno)benzoic acid |
| MTBE | methyl tert-butyl ether |
| MeOH | methanol |
| MeCN | acetonitrile |
| MS | mass spectrometry |
| MsCl | methanesulfonyl chloride |
| NMI | 1-methylimidazole |
| NMR | nuclear magnetic resonance |
| PCC | pyridinium chlorochromate |
| PE | petroleum ether |
| PMA | phosphomolybdic acid |
| PMBCl | p-methoxybenzyl chloride |
| Pht | phthaloyl |
| PyBOP | (benzotriazol-1- |
| | yloxytripyrrolidinophos- |
| | phonium hexafluorophosphate) |
| t-BuLi | tert-butylithium |
| TCFH | N,N,N′,N′- |
| | tetramethylchloroformamidi- |
| | nium hexafluorophosphate |
| T₃P | propanephosphonic acid |
| | anhydride |
| TEA | triethylamine |
| THF | tetrahydrofuran |
| TFA | trifluoroacetic acid |
| TFAA | trifluoroacetic anhydride |
| THF | tetrahydrofuran |
| Ti(i-PrO)₄ | titanium isopropoxide |
| TMSCN | trimethylsilyl cyanide |
| ZnEt₂ | diethylzinc |

Compounds of Tables 1, 2, 2a and 3 are prepared by following examples below.

### General Chemistry

Exemplary compounds described herein are available by the general synthetic method illustrated in Scheme 1 or 2, including preparations of Intermediates and preparation of accompanying Examples.

### Synthetic Scheme(s)

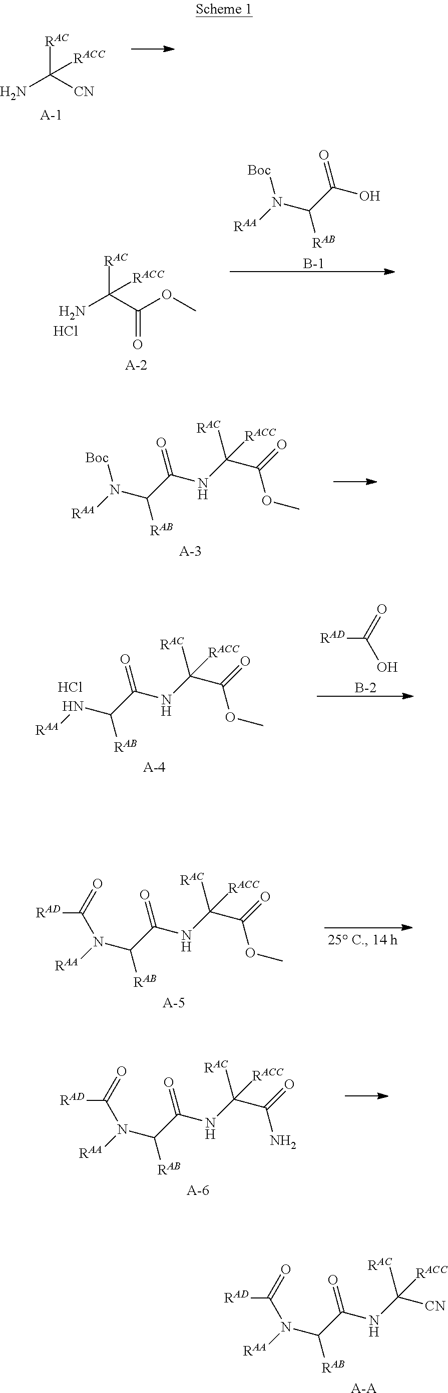

Scheme 1 illustrates an exemplary preparation of A-A. A Pinner reaction of amine A-1 with a reagent such as methanol affords amino ester A-2. A-2 is then coupled with carboxyxlic acid B-1 in the presence of reagents such as 1-ethyl-3-(3-dimethylaminopropyl)carbodiimide and 4-dimethylaminopyridine to afford the amide product A-3. Removal of the carbamate protecting group in A-3 provides amine A-4, which is coupled with carboxylic acid B-2 to afford amide A-5. Finally, the carboxylic acid group A-5 is converted to an amide (A-6), which is then converted to nitrile A-A in the presence of, for example, Burgess reagent.

In Scheme 1, examples of A^{C} and A^{CC} each independently include a hydrogen, a substituted or unsubstituted alkyl, a substituted or unsubstituted aryl, a substituted or unsubstituted monocylic heteroaryl moiety, and a substituted or unsubstituted bicyclic heteroaryl moiety, or A^{C} and A^{CC}, together with the atoms they are attached to, may form a substituted or unsubstituted carbocycle, a substituted or unsubstituted monocyclic heterocycle, or a substituted or unsubstituted bicyclic heterocycle; examples of R^{AA} and R^{AB} each independently include a substituted or unsubstituted alkyl, a substituted or unsubstituted cycloalkyl, a substituted or unsubstituted aryl, a substituted or unsubstituted monocylic heteroaryl moiety, and a substituted or unsubstituted bicyclic heteroaryl moiety, or R^{AA} and R^{AB}, together with the atoms they are attached to, form a ring, wherein the ring may be a substituted or unsubstituted heterocycle; and examples of R^{AD} include a substituted or unsubstituted bicyclic heteroaryl moiety.

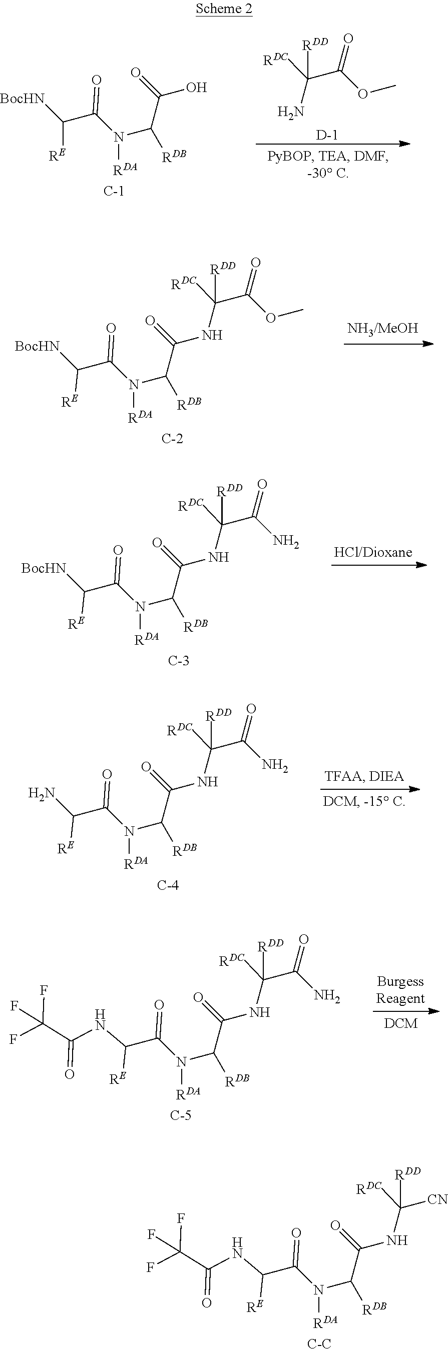

Scheme 2 illustrates an exemplary preparation of C—C. Carboxylic acid C-1 is coupled with amine D-1 in the presence of couple reagents such as benzotriazol-1-yloxytripyrrolidinophosphonium hexafluorophosphate and triethylamine to afford amide C-2. The ester in C-2 is then converted to its corresponding amide (C-3) in the presence of, for example, NH₃ and methanol. Removal of the carbamate protecting group in C-3 provides amine C-4, which is then converted to trifluoracetamide C-5. Dehydration of the primary amide in C-5 in the presence of, for example, Burgess Reagent, yields nitrile C—C.

In Scheme 2, examples of R^{DA} and^{DB} each independently include a substituted or unsubstituted alkyl, a substituted or unsubstituted cycloalkyl, a substituted or unsubstituted aryl, a substituted or unsubstituted monocylic heteroaryl moiety, and a substituted or unsubstituted bicyclic heteroaryl moiety, or RDA DB, together with the atoms they are attached to, form a ring, wherein the ring may be a substitued or unsubstituted heterocycle; examples of R^{E} include a substituted or unsubstituted alkyl; examples of R^{DC} and^{DD} each independently include a hydrogen, a substituted or unsubstituted alkyl, a substituted or unsubstituted aryl, a substituted or unsubstituted monocylic heteroaryl moiety, and a substituted or unsubstituted bicyclic heteroaryl moiety, or R^{DC} and ^{DD}, together with the atoms they are attached to, may form a substituted or unsubstituted carbocycle, a substituted or unsubstituted monocyclic heterocycle, or a substituted or unsubstituted bicyclic heterocycle; and examples of R^{°E} include a substituted or unsubstituted alkyl.

Example 1: Synthesis of (1R,2S,5S)—N-[(1S)-1-cyano-2-[(3S)-2-oxopyrrolidin-3-yl]ethyl]-3-[(2S)-3,3-dimethyl-2-(pyrazine-2-carbonylamino)butanoyl]-6,6-dimethyl-3-azabicyclo[3.1.0]hexane-2-carboxamide (Compound 101)

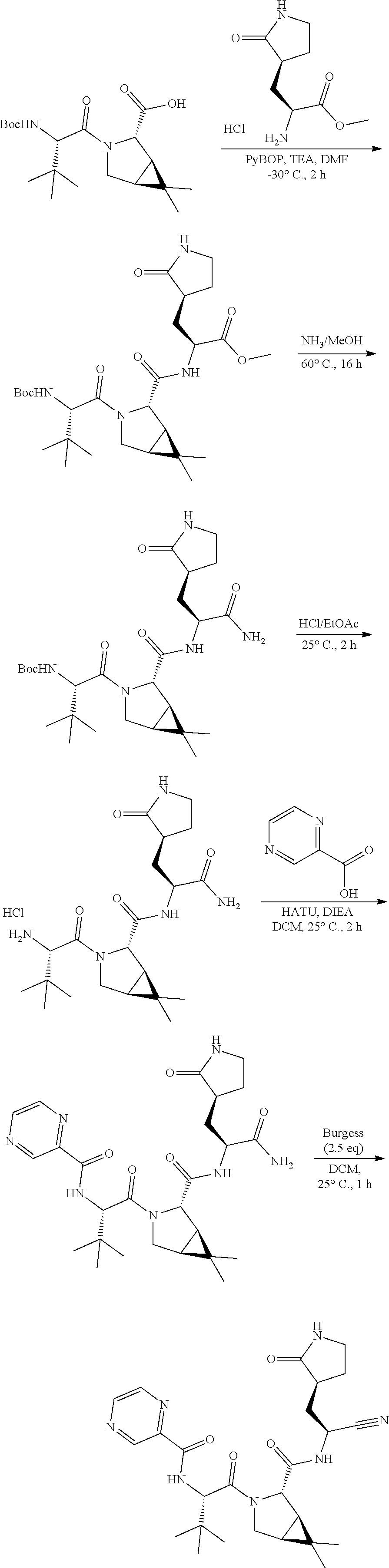

Step 1: methyl (2S)-2-[[(1R,2S,5S)-3-[(2S)-2-(tert-butoxycarbonylamino)-3,3-dimethyl-butanoyl]-6,6-dimethyl-3-azabicyclo[3.1.0]hexane-2-carbonyl]amino]-3-[(3S)-2-oxopyrrolidin-3-yl]propanoate

To a mixture of (1R,2S,5S)-3-[(2S)-2-(tert-butoxycarbonylamino)-3,3-dimethyl-butanoyl]-6,6-dimethyl-3-azabicyclo[3.1.0]hexane-2-carboxylic acid (300 mg, 814.19 umol, 1 eq), methyl (2S)-2-amino-3-[(3S)-2-oxopyrrolidin-3-yl]propanoate (235.68 mg, 1.06 mmol, 1.3 eq, HCl) and PyBOP (466.07 mg, 895.60 umol, 1.1 eq) in DMF (10 mL) at −30° C. was added TEA (164.77 mg, 1.63 mmol, 226.65 uL, 2 eq) drop-wise at −30° C. After the addition, the resulting reaction mixture was stirred at −30° C. for 2 h. Upon completion, the reaction mixture was diluted by water (30 mL) and extracted with EtOAc (20 mL*2). The combined organic layers were washed with brine (10 mL*2), dried over Na₂SO₄, concentrated in a vacuum to afford crude product. The crude product was purified by MPLC (100-200 silica gel, PE/EtOAc=1/1 to DCM/MeOH=10/1) to afford methyl (2S)-2-[[(1R,2S,5S)-3-[(2S)-2-(tert-butoxycarbonylamino)-3,3-dimethyl-butanoyl]-6,6-dimethyl-3-azabicyclo[3.1.0]hexane-2-carbonyl]amino]-3-[(3S)-2-oxopyrrolidin-3-yl]propanoate (400 mg, 686.32 umol, 84.30% yield, 92.08% purity) as white solid. MS (ESI) m/z 537.3 [M+H]⁺

Step 2: tert-butyl N-[(1S)-1-[(1R,2S,5S)-2-[[(1S)-2-amino-2-oxo-1-[[(3S)-2-oxopyrrolidin-3-yl]methyl]ethyl]carbamoyl]-6,6-dimethyl-3-azabicyclo[3.1.0]hexane-3-carbonyl]-2,2-dimethyl propyilcarbamate

A solution of methyl (2S)-2-[[(1R,2S,5S)-3-[(2S)-2-(tert-butoxycarbonylamino)-3,3-dimethyl-butanoyl]-6,6-dimethyl-3-azabicyclo[3.1.0]hexane-2-carbonyl]amino]-3-[(3S)-2-oxopyrrolidin-3-yl]propanoate (300 mg, 559.01 umol, 1 eq) in NH₃/MeOH (7 M, 6 mL, 75.13 eq) was stirred at 60° C. for 16 h. Upon completion, the reaction mixture was concentrated in a vacuum to dryness to afford tert-butyl N-[(1S)-1-[(1R,2S,5S)-2-[[(1S)-2-amino-2-oxo-1-[[(3S)-2-oxopyrrolidin-3-yl]methyl]athyl]carbamoyl]-6,6-dimethyl-3-azabicyclo[3.1.0]hexane-3-carbonyl]-2,2-dimethyl-propyl]carbamate (300 mg, crude) as yellow solid, which was used for next step without further purification.

Step 3: (1R,2S,5S)-3-[(2S)-2-amino-3,3-dimethyl-butanoyl]-N-[(1S)-2-amino-2-oxo-1-[[(3S)-2-oxopyrrolidin-3-yl]methyl]ethyl]-6,6-dimethyl-3-azabicyclo[3.1.0]hexane-2-carboxamide

A mixture of tert-butyl N-[(1S)-1-[(1R,2S,5S)-2-[[(1S)-2-amino-2-oxo-1-[[(3S)-2-oxopyrrolidin-3-yl]methyl]ethyl]carbamoyl]-6,6-dimethyl-3-azabicyclo[3.1.0]hexane-3-carbonyl]-2,2-dimethyl-propyl]carbamate (235 mg, 450.50 umol, 1 eq) in HCl/EtOAc (4 M, 5 mL, 44.40 eq) was stirred at 25° C. for 2 h. Upon completion, the reaction mixture was concentrated in a vacuum to dyness to afford (1R,2S,5S)-3-[(2S)-2-amino-3,3-dimethyl-butanoyl]-N-[(1S)-2-amino-2-oxo-1-[[(3S)-2-oxopyrrolidin-3-yl]methyl]ethyl]-6,6-dimethyl-3-azabicyclo[3.1.0]hexane-2-carboxamide (220 mg, crude, HCl) as white solid. MS (ESI) m/z 422.3 [M+H]⁺

Step 4: (1R,2S,5S)—N-[(1S)-2-amino-2-oxo-1-[[(3S)-2-oxopyrrolidin-3-yl]methyl]ethyl]-3-[(2S)-3,3-dimethyl-2-(pyrazine-2-carbonylamino)butanoyl]-6,6-dimethyl-3-azabicyclo[3.1.0]hexane-2-carboxamide

To a mixture of pyrazine-2-carboxylic acid (59.61 mg, 480.36 umol, 1.1 eq) in DCM (10 mL) was added HATU (249.06 mg, 655.03 umol, 1.5 eq), DIEA (169.32 mg, 1.31 mmol, 228.19 uL, 3 eq) and (1R,2S,5S)-3-[(2S)-2-amino-3,3-dimethyl-butanoyl]-N-[(1S)-2-amino-2-oxo-1-[[(3S)-2-oxopyrrolidin-3-yl]methyl]ethyl]-6,6-dimethyl-3-azabicyclo[3.1.0]hexane-2-carboxamide (200 mg, 436.69 umol, 1 eq, HCl) in turn. After the addition, the reaction mixture was stirred at 25° C. for 2 h. Upon completion, the reaction mixture was diluted by DCM (10 mL) and quenched by 5 mL 10% citric acid (aq.). The organic layer was separated and purified by prep-TLC to afford (1R,2S,5S)—N-[(1S)-2-amino-2-oxo-1-[[(3S)-2-oxopyrrolidin-3-yl]methyl]ethyl]-3-[(2S)-3,3-dimethyl-2-(pyrazine-2-carbonylamino)butanoyl]-6,6-dimethyl-3-azabicyclo[3.1.0]hexane-2-carboxamide (120 mg, 227.44 umol, 52.08% yield) as white solid. MS (ESI) m/z 528.3 [M+H]⁺

Step 5: (1R,2S,5S)—N-[(1S)-1-cyano-2-[(3S)-2-oxopyrrolidin-3-yl]ethyl]-3-[(2S)-3,3-dimethyl-2-(pyrazine-2-carbonylamino)butanoyl]-6,6-dimethyl-3-azabicyclo[3.1.0]hexane-2-carboxamide

To a solution of (1R,2S,5S)—N-[(1S)-2-amino-2-oxo-1-[[(3S)-2-oxopyrrolidin-3-yl]methyl]ethyl]-3-[(25)-3,3-dimethyl-2-(pyrazine-2-carbonylamino)butanoyl]-6,6-dimethyl-3-azabicyclo[3.1.0]hexane-2-carboxamide (100 mg, 189.53 umol, 1 eq) in DCM (1 mL) was added Burgess reagent (112.92 mg, 473.83 umol, 2.5 eq) at 25° C. After the addition, the reaction mixture was stirred at 25° C. for 1 h. Upon completion, the reaction mixture was quenched by 0.5 mL water and blow to dryness with N2. The resulting mixture was diluted by DMF (1.5 mL). The resulting mixture was purified by prep-HPLC (column: Phenomenex Luna C18 75*30 mm*3 um; mobile phase: [water(FA)-ACN]; B %: 20%-60%, 8 min) to afford (1R,2S,5S)—N-[(1S)-1-cyano-2-[(3S)-2-oxopyrrolidin-3-yl]ethyl]-3-[(2S)-3,3-dimethyl-2-(pyrazine-2-carbonylamino)butanoyl]-6,6-dimethyl-3-azabicyclo[3.1.0]hexane-2-carboxamide (Isomer 1) (40 mg, 71.51 umol, 37.73% yield, 91.1% purity) as a white solid, and (1R,2S,5S)—N-[(1S)-1-cyano-2-[(3S)-2-oxopyrrolidin-3-yl]ethyl]-3-[(2S)-3,3-dimethyl-2-(pyrazine-2-carbonylamino)butanoyl]-6,6-dimethyl-3-azabicyclo[3.1.0]hexane-2-carboxamide (Isomer 2) (5 mg, 8.40 umol, 4.43% yield, 85.6% purity) as white solid. MS (ESI) m/z 510.2 [M+H]⁺

### Isomer 1:

¹H NMR (400 MHz, DMSO-d₆) δ=9.23-9.18 (m, 1H), 9.06 (d, J=8.1 Hz, 1H), 8.94-8.89 (m, 1H), 8.76 (dd, J=1.5, 2.4 Hz, 1H), 8.26-8.06 (m, 1H), 7.84-7.68 (m, 1H), 5.04-4.82 (m, 1H), 4.60 (d, J=9.4 Hz, 1H), 4.17 (s, 1H), 3.98 (dd, J=5.3, 10.3 Hz, 1H), 3.81 (d, J=10.5 Hz, 1H), 3.20-3.12 (m, 1H), 3.10-3.01 (m, 1H), 2.40 (br dd, J=4.7, 8.4 Hz, 1H), 2.20-2.05 (m, 2H), 1.78-1.68 (m, 2H), 1.62-1.54 (m, 1H), 1.34 (d, J=7.7 Hz, 1H), 1.03 (s, 3H), 1.01 (s, 9H), 0.83 (s, 3H)

¹H NMR (400 MHz, DMSO-d₆, 273+80K) δ=9.21-9.15 (m, 1H), 8.86 (d, J=2.4 Hz, 1H), 8.83-8.78 (m, 1H), 8.71-8.69 (m, 1H), 8.19-8.11 (m, 1H), 7.47-7.40 (m, 1H), 4.99-4.91 (m, 1H), 4.61 (d, J=9.7 Hz, 1H), 4.21 (s, 1H), 4.00-3.94 (m, 1H), 3.83 (d, J=10.4 Hz, 1H), 3.24-3.10 (m, 2H), 2.42-2.37 (m, 1H), 2.22-2.11 (m, 2H), 1.82-1.68 (m, 2H), 1.60-1.53 (m, 1H), 1.31 (d, J=7.7 Hz, 1H), 1.04-1.00 (m, 12H), 0.94 (s, 2H), 0.81 (s, 3H)

### Isomer 2:

¹H NMR (400 MHz, DMSO-d₆) δ=9.21-9.14 (m, 1H), 9.03 (d, J=8.5 Hz, 1H), 8.93-8.87 (m, 1H), 8.73 (dd, J=1.5, 2.4 Hz, 1H), 8.24-8.05 (m, 1H), 7.80-7.63 (m, 1H), 5.01-4.79 (m, 1H), 4.58 (d, J=9.2 Hz, 1H), 4.15 (s, 1H), 4.01-3.91 (m, 1H), 3.78 (d, J=10.5 Hz, 1H), 3.18-3.10 (m, 1H), 3.04 (dt, J=7.2, 9.2 Hz, 1H), 2.43-2.32 (m, 1H), 2.21 1.96 (m, 2H), 1.77-1.65 (m, 2H), 1.58 (dd, J=5.4, 7.6 Hz, 1H), 1.41-1.28 (m, 1H), 1.03 1.00 (m, 3H), 0.99-0.90 (m, 9H), 0.80 (s, 3H)

¹H NMR (400 MHz, DMSO-d₆, 273+80K) 8=9.23-9.15 (m, 1H), 8.89-8.86 (m, 1H), 8.84-8.80 (m, 1H), 8.72 (dd, J=1.7, 2.3 Hz, 1H), 8.20-8.13 (m, 1H), 7.44 (br d, J=2.0 Hz, 1H), 5.01-4.93 (m, 1H), 4.63 (d, J=9.7 Hz, 1H), 4.22 (s, 1H), 4.02-3.95 (m, 1H), 3.88-3.82 (m, 1H), 3.22-3.10 (m, 2H), 2.44-2.36 (m, 1H), 2.23-2.12 (m, 2H), 1.83 1.71 (m, 2H), 1.63-1.56 (m, 1H), 1.33 (d, J=7.7 Hz, 1H), 1.06-1.02 (m, 12H), 0.83 (s, 3H)

Example 2: Synthesis of N-[(1S)-1-[[(1S)-2-[[(1S)-1-cyan-2-[(3S)-2-oxo-3-piperidyl]ethyl]amino]-1-(cyclopropylmethyl)-2-oxo-ethyl]-methyl-carbamoyl]-2,2-dimethyl-propyl]pyrazine-2-carboxamide (Compound 102)

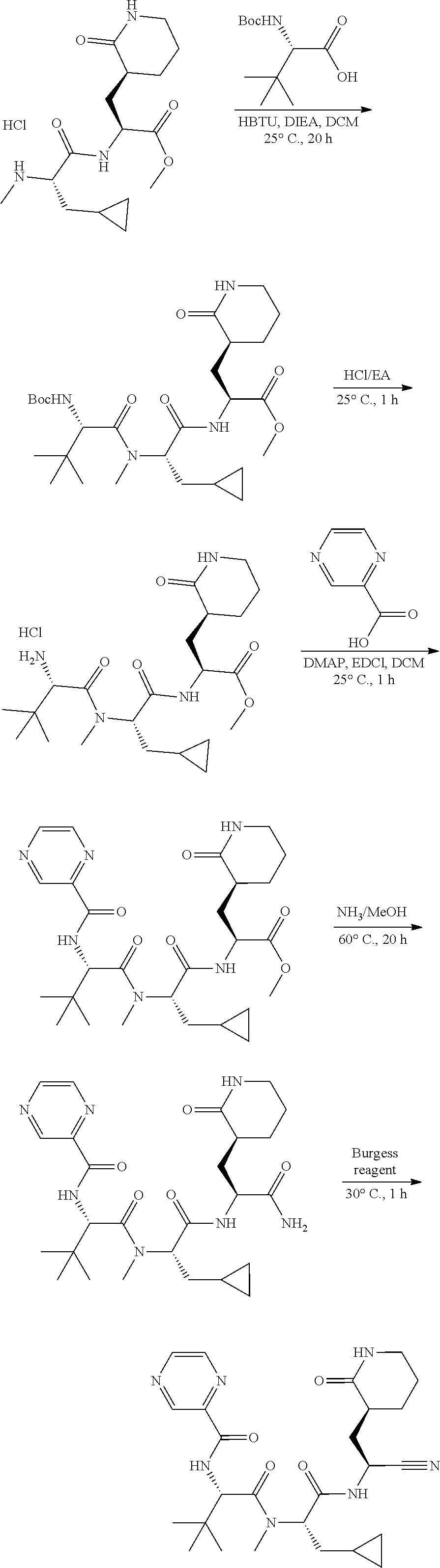

Step 1: methyl (2S)-2-[[(2S)-2-[[(2S)-2-(tert-butoxycarbonylamino)-3,3-dimethyl-butanoyl]-methyl-amino]-3-cyclopropyl-propanoyl]amino]-3-[(3S)-2-oxo-3-piperidyl]propanoate

To a solution of methyl (2S)-2-[[(2S)-3-cyclopropyl-2-(methylamino)propanoyl]amino]-3-[(3S)-2-oxo-3-piperidyl]propanoate (156.46 mg, 4 batches in parallel, 432.36 umol, 1.0 eq, HCl) in DCM (2 mL) was added HBTU (180.37 mg, 475.60 umol, 1.1 eq), (2S)-2-(tert-butoxycarbonylamino)-3,3-dimethyl-butanoic acid (100 mg, 432.36 umol, 1.00 eq) and DIEA (111.76 mg, 864.72 umol, 150.62 uL, 2 eq). The mixture was stirred at 25° C. for 20 h. Upon completion, the reaction mixture was diluted with water (50 mL) and extracted with DCM (10 mL*3). The combined organic layers were dried over Na₂SO₄, filtered and concentrated under reduced pressure to give a residue. The residue was purified by column (SiO₂, Petroleum ether:Ethyl acetate=2:1-0:1) to get product methyl (2S)-2-[[(2S)-2-[[(2S)-2-(tert-butoxycarbonylamino)-3,3-dimethyl-butanoyl]-methyl-amino]-3-cyclopropyl-propanoyl]amino]-3-[(3S)-2-oxo-3-piperidyl]propanoate (500 mg, 928.20 umol, 53.67% yield) as white oil. MS (ESI) m/z 539.3 [M+H]⁺.

Step 2: methyl (2S)-2-[[(2S)-2-[[(2S)-2-amino-3,3-dimethyl-butanoyl]-methyl-amino]-3-cyclopropyl-propanoyl]amino]-3-[(3S)-2-oxo-3-piperidyl]propanoate

A solution of methyl (2S)-2-[[(2S)-2-[[(2S)-2-(tert-butoxycarbonylamino)-3,3-dimethyl-butanoyl]-methyl-amino]-3-cyclopropyl-propanoyl]amino]-3-[(3S)-2-oxo-3-piperidyl]propanoate (500 mg, 928.20 umol, 1 eq) in HCl/EtOAc (4 M, 6 mL, 25.86 eq) was stirred at 25° C. for 1 h. Upon completion, the mixture was concentrated under reduced pressure to give a residue, then was dissolved with DCM (10 mL*3) and concentrated under reduced pressure to get the product methyl (2S)-2-[[(2S)-2-[[(2S)-2-amino-3,3-dimethyl-butanoyl]-methyl-amino]-3-cyclopropyl-propanoyl]amino]-3-[(3S)-2-oxo-3-piperidyl]propanoate (440 mg, crude, HCl) as white solid MS (ESI) m/z 439.2 [M+H]⁺.

Step 3: methyl (2S)-2-[[(2S)-3-cyclopropyl-2-[[(2S)-3,3-dimethyl-2-(pyrazine-2-carbonylamino)butanoyl]-methyl-amino]propanoyl]amino]-3-[(3S)-2-oxo-3-piperidyl]propanoate

To a solution of methyl (2S)-2-[[(2S)-2-[[(2S)-2-amino-3,3-dimethyl-butanoyl]-methyl-amino]-3-cyclopropyl-propanoyl]amino]-3-[(3S)-2-oxo-3-piperidyl]propanoate (480 mg, 1.01 mmol, 1 eq, HCl) in DCM (10 mL) was added pyrazine-2-carboxylic acid (225.72 mg, 1.82 mmol, 1.8 eq), DMAP (370.35 mg, 3.03 mmol, 3 eq) and EDCI (484.28 mg, 2.53 mmol, 2.5 eq). The mixture was stirred at 25° C. for 1 h. Upon completion, the reaction mixture was diluted with water (30 mL) and extracted with DCM (10 mL*3). The combined organic layers were dried over Na₂SO₄, filtered and concentrated under reduced pressure to give a residue. The residue was purified by column (SiO₂, Petroleum ether:Ethyl acetate=2:1-0:1) to get the product methyl (2S)-2-[[(2S)-3-cyclopropyl-2-[[(2S)-3,3-dimethyl-2-(pyrazine-2-carbonylamino)butanoyl]-methyl-amino]propanoyl]amino]-3-[(3S)-2-oxo-3-piperidyl]propanoate (300 mg, 413.12 umol, 40.88% yield, 75% purity) as yellow solid. MS (ESI) m/z 545.3 [M+H]⁺.

Step 4: N-[(1S)-1-[[(1S)-2-[[(1S)-2-amino-2-oxo-1-[[(3S)-2-oxo-3-piperidyl]methyl]ethyl]amino]-1-(cyclopropylmethyl)-2-oxo-ethyl]-methyl-carbamoyl]-2,2-dimethyl-propyl]pyrazine-2-carboxamide

A solution of methyl (2S)-2-[[(2S)-3-cyclopropyl-2-[[(2S)-3,3-dimethyl-2-(pyrazine-2-carbonylamino)butanoyl]-methyl-amino]propanoyl]amino]-3-[(3S)-2-oxo-3-piperidyl]propanoate (270 mg, 371.80 umol, 75% purity, 1 eq) in NH₃/MeOH (7 M, 3 mL, 56.48 eq) was stirred at 60° C. for 20 h. Upon completion, the mixture was concentrated under reduced pressure to give a residue, then was dissolved with DCM (6 mL*3) and concentrated under reduced pressure to get the product N-[(1S)-1-[[(1S)-2-[[(1S)-2-amino-2-oxo-1-[[(3S)-2-oxo-3-piperidyl]methyl]ethyl]amino]-1-(cyclopropylmethyl)-2-oxo-ethyl]-methyl-carbamoyl]-2,2-dimethyl-propyl]pyrazine-2-carboxamide (190 mg, crude) as yellow solid. MS (ESI) m/z 530.3 [M+H]⁺.

Step 5: N-[(1S)-1-[[(1S)-2-[[(1S)-1-cyano-2-[(3S)-2-oxo-3-piperidyl]ethyl]amino]-1-(cyclopropylmethyl)-2-oxo-ethyl]-methyl-carbamoyl]-2,2-dimethyl-propyl]pyrazine-2-carboxamide

To a mixture of N-[(1S)-1-[[(1S)-2-[[(1S)-2-amino-2-oxo-1-[[(3S)-2-oxo-3-piperidyl]methyl]ethyl]amino]-1-(cyclopropylmethyl)-2-oxo-ethyl]-methyl-carbamoyl]-2,2-dimethyl-propyl]pyrazine-2-carboxamide (190 mg mg, 358.74 umol, 1 eq) in DCM (2 mL) was added Burgess reagent (256.47 mg, 1.08 mmol, 3 eq). The mixture was stirred at 30° C. for 1 h. Upon completion, the mixture were quenched with water (1 mL) and blow-dried with N2. The residue was purified by prep-HPLC (column: Phenomenex Luna C18 75*30 mm*3 um; mobile phase: [water (FA)-ACN]; B %: 25%-55%, 8 min) to get the product N-[(1S) 1-[[(1S)-2-[[(1S)-1-cyano-2-[(3S)-2-oxo-3-piperidyl]ethyl]amino]-1-(cyclopropylmethyl)-2-oxo-ethyl]-methyl-carbamoyl]-2,2-dimethyl-propyl]pyrazine-2-carboxamide (69.83 mg, 136.49 umol, 38.05% yield, 100% purity) as white solid. MS (ESI) m/z 510.3 [M+H]⁺.

¹H NMR (400 MHz, DMSO-d₆) δ=9.23-9.13 (m, 1H), 8.95-8.81 (m, 2H), 8.76 (br s, 1H), 8.30 (br d, J=8.9 Hz, 1H), 7.50 (br s, 1H), 5.07-4.70 (m, 3H), 3.16-2.79 (m, 5H), 2.29-2.04 (m, 2H), 1.81-1.44 (m, 6H), 1.40-1.24 (m, 1H), 1.07-0.92 (m, 9H), 0.57-0.49 (m, 1H), 0.31-0.21 (br s, 2H), 0.04 (br s, 2H).

¹H NMR (400 MHz, DMSO-d₆, 273+80K) δ=9.20 (s, 1H), 8.87 (br s, 1H), 8.72 (brd, J=1.1 Hz, 1H), 8.66-8.52 (m, 1H), 8.45-8.12 (m, 1H), 7.28-7.06 (m, 1H), 5.15 4.66 (m, 3H), 3.25-2.8 (m, 5H), 2.21 (br s, 2H), 1.96-1.51 (m, 6H), 1.49-1.33 (m, 1H), 1.05 (s, 9H), 0.65-0.50 (m, 1H), 0.46-0.23 (m, 2H), 0.06 (br s, 2H).

Example 3: Synthesis of benzyl((S)-1-(((S)-1-(((S)-1-cyano-2-((R)-5-oxo-4-azaspiro[2.4]heptan-6-yl)ethyl)amino)-3-cyclopropyl-1-oxopropan-2-yl)amino)-3-(naphthalen-1-yl)-1-oxopropan-2-yl)carbamate (Compound 110)

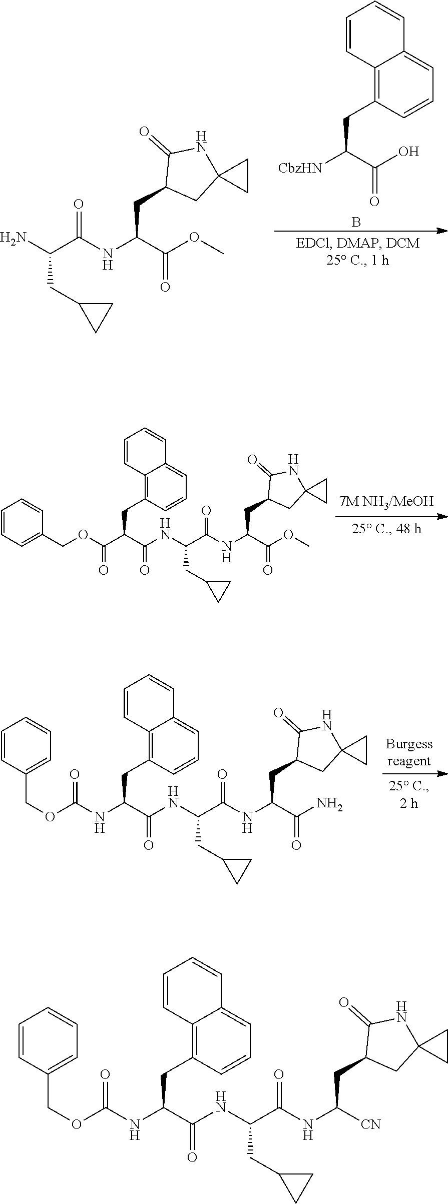

Step 1: (S)-benzyl3-(((S)-3-cyclopropyl-1-(((S)-1-methoxy-1-oxo-3-((R)-5-oxo-4-azaspiro[2.4]heptan-6-yl)propan-2-yl)amino)-1-oxopropan-2-yl)amino)-2-(naphthalen-1-ylmethyl)-3-oxopropanoate

To a solution of methyl (2S)-2-[[(2S)-2-amino-3-cyclopropyl-propanoyl]amino]-3-[(6R)-5-oxo-4-azaspiro[2.4]heptan-6-yl]propanoate (500 mg, 1.39 mmol, 1 eq, HCl) and (2S)-2-(benzyloxycarbonylamino)-3-(1-naphthyl)propanoic acid (582.55 mg, 1.67 mmol, 1.2 eq) in DCM (20 mL) was added DMAP (339.50 mg, 2.78 mmol, 2 eq) and EDCI (532.73 mg, 2.78 mmol, 2 eq). The mixture was stirred at 25° C. for 1 h. Upon completion, the reaction mixture was diluted by addition H2O (30 mL), and then extracted with DCM (20 mL*3). The combined organic layers were washed with brine (30 mL), dried over Na₂SO₄, filtered and concentrated under reduced pressure to give a residue. The residue was purified by column chromatography (SiO₂, Petroleum ether:Ethyl acetate=10:1 to 0:1) to give the product methyl (2S)-2-[[(2S)-2-[[(2S)-2-(benzyloxycarbonylamino)-3-(1-naphthyl)propanoyl]amino]-3-cyclopropyl-propanoyl]amino]-3-[(6R)-5-oxo-4-azaspiro[2.4]heptan-6-yl]propanoate (750 mg, 1.15 mmol, 82.44% yield) as a white solid. MS (ESI) m/z 655.3 [M+H]⁺

Step 2: benzyl((S)-1-(((S)-1-(((S)-1-amino-1-oxo-3-((R)-5-oxo-4-azaspiro[2.4]heptan-6-yl)propan-2-yl)amino)-3-cyclopropyl-1-oxopropan-2-yl)amino)-3-(naphthalen-1-yl)-1-oxopropan-2-yl)carbamate

A mixture of methyl (2S)-2-[[(2S)-2-[[(2S)-2-(benzyloxycarbonylamino)-3-(1-naphthyl)propanoyl]amino]-3-cyclopropyl-propanoyl]amino]-3-[(6R)-5-oxo-4-azaspiro[2.4]heptan-6-yl]propanoate (150 mg, 229.09 umol, 1 eq) in NH₃/MeOH (7 M, 10 mL, 305.55 eq) was stirred at 25° C. for 48 h. Upon completion, the mixture was concentrated under the reduced pressure to give a the product benzyl N-[(1S)-2-[[(1S)-2-[[(1S)-2-amino-2-oxo-1-[[(6R)-5-oxo-4-azaspiro[2.4]heptan-6-yl]methyl]ethyl]amino]-1-(cyclopropylmethyl)-2-oxo-ethyl]amino]-1-(1-naphthylmethyl)-2-oxo-ethyl]carbamate (130 mg, crude) as a yellow solid. MS (ESI) m/z 640.3 [M+H]⁺

Step 3: benzyl((S)-1-(((S)-1-(((S)-1-cyano-2-((R)-5-oxo-4-azaspiro[2.4]heptan-6-yl)ethyl)amino)-3-cyclopropyl-1-oxopropan-2-yl)amino)-3-(naphthalen-1-yl)-1-oxopropan-2-yl)carbamate

To a solution of benzyl N-[(1S)-2-[[(1S)-2-[[(1S)-2-amino-2-oxo-1-[[(6R)-5-oxo-4-azaspiro[2.4]heptan-6-yl]methyl]ethyl]amino]-1-(cyclopropylmethyl)-2-oxo-ethyl]amino]-1-(1-naphthylmethyl)-2-oxo-ethyl]carbamate (120 mg, 187.58 umol, 1 eq) in DCM (5 mL) was added Burgess reagent (134.10 mg, 562.73 umol, 3 eq). The mixture was stirred at 20° C. for 24 h. Upon completion, the mixture was diluted with H2O (0.5 mL) and then drying with N2 to give a residue. The residue was purified by prep-HPLC (column: Waters Xbridge Prep OBD C18 150*40 mm*10 um; mobile phase: [water (NH₄HCO₃)-ACN]; B %: 45%-65%, 8 min) to give the product benzyl N-[(1S)-2-[[(1S)-2-[[(1S)-1-cyano-2-[(6R)-5-oxo-4-azaspiro[2.4]heptan-6-yl]ethyl]amino]-1-(cyclopropylmethyl)-2-oxo-ethyl]amino]-1-(1-naphthylmethyl)-2-oxo-ethyl]carbamate (30 mg, 48.25 umol, 25.72% yield, 100% purity) as a white solid. MS (ESI) m/z 622.3 [M+H]⁺

¹H NMR (400 MHz, MeOD-d₄) δ=8.18 (br d, J=8.4 Hz, 1H), 7.86 (br d, J=7.6 Hz, 1H), 7.85-7.76 (m, 1H), 7.55-7.45 (m, 2H), 7.41-7.16 (m, 7H), 5.10-4.91 (m, 3H), 4.59-4.56 (m, 1H), 4.31-4.29 (m, 1H), 3.70-3.65 (m, 1H), 3.31-3.30 (m, 1H), 2.75 2.65 (m, 1H), 2.48-2.37 (m, 1H), 2.18-2.11 (m, 1H), 2.08-2.03 (m, 1H), 2.01-1.92 (m, 1H), 1.72-1.67 (m, 1H), 1.56-1.44 (m, 1H), 0.83-0.79 (m, 1H), 0.73-0.61 (m, 4H), 0.50 0.42 (m, 2H), 0.15-0.06 (m, 2H)

Example 4: Synthesis of N—((S)-1-(((S)-1-(((S)-1-cyano-2-((S)-2-oxopiperidin-3-yl) ethyl) amino)-4-methyl-1-oxopentan-2-yl) amino)-3-(4-tluorophenyl)-1-oxopropan-2-yl)-5-methylisoxazole-3-carboxamide (Compound 113)

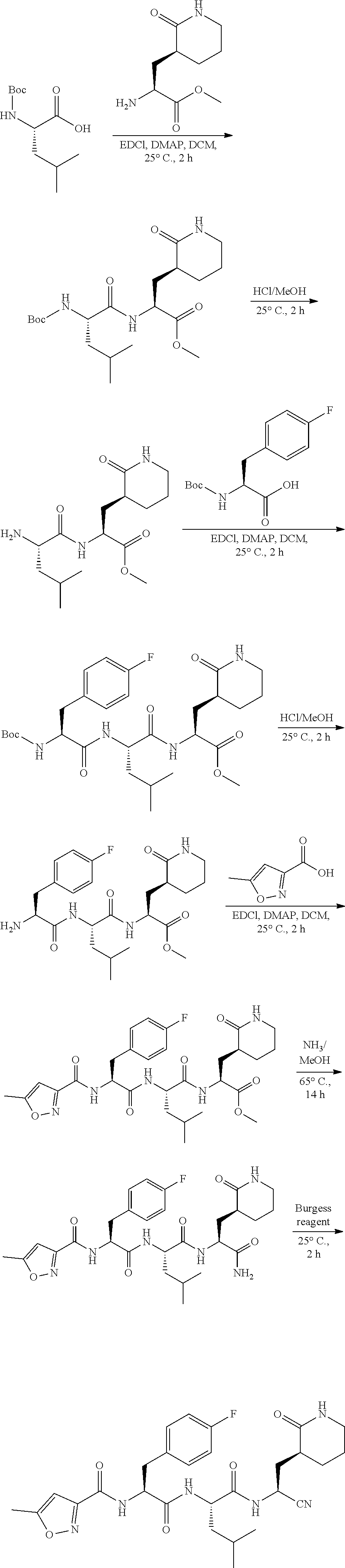

Step 1: (S)-methyl 2-((S)-2-((tert-butoxycarbonyl)amino)-4-methylpentanamido)-3-((S)-2-oxopiperidin-3-yl)propanoate

A solution of (2S)-2-(tert-butoxycarbonylamino)-4-methyl-pentanoic acid (5 g, 21.62 mmol, 1 eq), methyl (2S)-2-amino-3-[(3S)-2-oxo-3-piperidyl]propanoate (4.76 g, 23.78 mmol, 1.1 eq) in DCM (200 mL) was added DMAP (5.28 g, 43.24 mmol, 2 eq) and EDCI (6.22 g, 32.43 mmol, 1.5 eq). The mixture was stirred at 25° C. for 2 h. Upon completion, the reaction mixture was quenched by addition H2O 150 mL, and then extracted with DCM (80 mL*3). The combined organic layers were washed with brine 100 mL, dried over Na₂SO₄, filtered and concentrated under reduced pressure to give a residue. The residue was purified by column chromatography (SiO₂, Petroleum ether/Ethyl acetate=2/1 to 0/1) to give methyl (2S)-2-[[(2S)-2-(tert-butoxycarbonylamino)-4-methyl-pentanoyl]amino]-3-[(3S)-2-oxo-3-piperidyl]propanoate (6 g, 13.49 mmol, 62.42% yield, 93% purity) as a light yellow solid. MS (ESI) m/z 414.3 [M+H]⁺.

Step 2: (S)-methyl 2-((S)-2-amino-4-methylpentanamido)-3-((S)-2-oxopiperidin-3-yl)propanoate

A solution of methyl (2S)-2-[[(2S)-2-(tert-butoxycarbonylamino)-4-methyl-pentanoyl]amino]-3-[(3S)-2-oxo-3-piperidyl]propanoate (500 mg, 1.21 mmol, 1 eq) in HCl/MeOH (10 mL) was stirred at 25° C. for 2 h. Upon completion, the reaction mixture was concentrated under reduce pressure to remove HCl/MeOH to give a methyl (2S)-2-[[(2S)-2-amino-4-methyl-pentanoyl]amino]-3-[(3S)-2-oxo-3-piperidyl]propanoate (400 mg, crude) as a white solid. MS (ESI) m/z 314.2 [M+H]⁺.

Step 3: (6S,9S,12S)-methyl 6-(4-fluorobenzyl)-9-isobutyl-2,2-dimethyl-4,7,10-trioxo-12-(((S)-2-oxopiperidin-3-yl)methyl)-3-oxa-5,8,11-triazatridecan-13-oate

A solution of methyl (2S)-2-[[(2S)-2-amino-4-methyl-pentanoyl]amino]-3-[(3S)-2-oxo-3-piperidyl]propanoate (400 mg, 1.28 mmol, 1 eq), (2S)-2-(tert-butoxycarbonylamino)-3-(4-fluorophenyl)propanoic acid (361.59 mg, 1.28 mmol, 1 eq) in DCM (15 mL) was added DMAP (311.86 mg, 2.55 mmol, 2 eq) and EDCI (367.02 mg, 1.91 mmol, 1.5 eq). The mixture was stirred at 25° C. for 2 h. Upon completion, the reaction mixture was quenched by addition H2O 20 mL, and then extracted with DCM (30 mL*3). The combined organic layers were washed with brine 50 mL, dried over Na₂SO₄, filtered and concentrated under reduced pressure to give a residue. The residue was purified by column chromatography (SiO₂, Petroleum ether/Ethyl acetate=2/1 to 0/1) to give methyl (2S)-2 [[(2S)-2-[[(2S)-2-(tert-butoxycarbonylamino)-3-(4-fluorophenyl)propanoyl]amino]-4-methyl-pentanoyl]amino]-3-[(3S)-2-oxo-3-piperidyl]propanoate (454 mg, 753.17 umol, 59.01% yield, 96% purity) as a white solid. MS (ESI) m/z 579.3 [M+H]⁺.

Step 4: (S)-methyl 2-((S)-2-((S)-2-amino-3-(4-fluorophenyl)propanamido)-4-methylpentanamido)-3-((S)-2-oxopiperidin-3-yl)propanoate

A solution of methyl (2S)-2-[[(2S)-2-[[(2S)-2-(tert-butoxycarbonylamino)-3-(4-fluorophenyl) propanoyl]amino]-4-methyl-pentanoyl] amino]-3-[(3S)-2-oxo-3-piperidyl]propanoate (454 mg, 784.56 umol, 1 eq) in HCl/MeOH (10 mL) was stirred at 25° C. for 2 h. Upon completion, the reaction mixture was concentrated under reduce pressure to remove HCl/MeOH to give methyl (2S)-2-[[(2S)-2-[[(2S)-2-amino-3-(4-fluorophenyl) propanoyl] amino]-4-methyl-pentanoyl] amino]-3-[(3S)-2-oxo-3-piperidyl] propanoate (480 mg, crude) as a white solid. MS (ESI) m/z 479.3 [M+H]⁺.

Step 5: (S)-methyl 2-((S)-2-((S)-3-(4-fluorophenyl)-2-(5-methylisoxazole-3-carboxamido)propanamido)-4-methylpentanamido)-3-((S)-2-oxopiperidin-3-yl)propanoate

A solution of methyl (2S)-2-[[(2S)-2-[[(2S)-2-amino-3-(4-fluorophenyl)propanoyl]amino]-4-methyl-pentanoyl]amino]-3-[(3S)-2-oxo-3-piperidyl]propanoate (480 mg, 1.00 mmol, 1 eq), and 5-methylisoxazole-3-carboxylic acid (127.48 mg, 1.00 mmol, 1 eq) in DCM (13 mL) was added DMAP (245.07 mg, 2.01 mmol, 2 eq) and EDCI (288.42 mg, 1.50 mmol, 1.5 eq). The mixture was stirred at 25° C. for 2 h. Upon completion, the reaction mixture was quenched by addition H2O 20 mL, and then extracted with DCM (30 mL*3). The combined organic layers were washed with brine 50 mL, dried over Na₂SO₄, filtered and concentrated under reduced pressure to give a residue. The residue was purified by column chromatography (SiO₂, Petroleum ether/Ethyl acetate=5/1 to 0/1) to give methyl (2S)-2-[[(2S)-2-[[(2S)-3-(4-fluorophenyl)-2-[(5-methylisoxazole-3-carbonyl) amino] propanoyl] amino]-4-methyl-pentanoyl] amino]-3-[(3S)-2-oxo-3-piperidyl]propanoate (386 mg, 630.59 umol, 62.87% yield, 96% purity) as a white solid. MS (ESI) m/z 588.3 [M+H]⁺.

Step 6: N—((S)-1-(((S)-1-(((S)-1-amino-1-oxo-3-((S)-2-oxopiperidin-3-yl)propan-2-yl)amino)-4-methyl-1-oxopentan-2-yl)amino)-3-(4-fluorophenyl)-1-oxopropan-2-yl)-5-methylisoxazole-3-carboxamide

A solution of methyl (2S)-2-[[(2S)-2-[[(2S)-3-(4-fluorophenyl)-2-[(5-methylisoxazole-3-carbonyl)amino]propanoyl]amino]-4-methyl-pentanoyl]amino]-3-[(3S)-2-oxo-3-piperidyl]propanoate (210 mg, 357.36 umol, 1 eq) in NH₃MeOH (7 M, 6 mL, 117.53 eq) was stirred at 65° C. for 14 h. Upon completion, the mixture was concentrated under reduce pressure to remove NH₃MeOH and DCM 5 mL was added (three times) and then was concentrated under reduce pressure to give N-[(1S)-2-[[(1S)-1-[[(1S)-2-amino-2-oxo-1-[[(3S)-2-oxo-3-piperidyl]methyl]ethyl]carbamoyl]-3-methyl-butyl]amino]-1-[(4-fluorophenyl)methyl]-2-oxo-ethyl]-5-methyl-isoxazole-3-carboxamide (125 mg, crude) as a white solid. MS (ESI) m/z 573.3 [M+H]⁺.

Step 7: N—((S)-1-(((S)-1-(((S)-1-cyano-2-((S)-2-oxopiperidin-3-yl)ethyl)amino)-4-methyl-1-oxopentan-2-yl)amino)-3-(4-fluorophenyl)-1-oxopropan-2-yl)-5-methylisoxazole-3-carboxamide

A solution of N-[(1S)-2-[[(1S)-1-[[(1S)-2-amino-2-oxo-1-[[(3S)-2-oxo-3-piperidyl]methyl]ethyl]carbamoyl]-3-methyl-butyl]amino]-1-[(4-fluorophenyl)methyl]-2-oxo-ethyl]-5-methyl-isoxazole-3-carboxamide (125 mg, 218.29 umol, 1 eq) in DCM (5 mL) was added with Burgess reagent (156.06 mg, 654.88 umol, 3 eq). The mixture was stirred 25° C. for 2 h. Upon completion, the reaction mixture was concentrated to remove DCM under N2. The residue was purified by prep-HPLC (column: Phenomenex C18 75*30 mm 3 um; mobile phase: [water (NH₄HCO₃)-ACN]; B %: 25%-65%, 8 min) to give N-[(1S)-2 [[(1S)-1-[[(I S)-1-cyano-2-[(3S)-2-oxo-3-piperidyl]ethyl]carbamoyl]-3-methyl-butyl]amino]-1-[(4-fluorophenyl)methyl]-2-oxo-ethyl]-5-methyl-isoxazole-3-carboxamide (30 mg, 53.55 umol, 24.53% yield, 99% purity) as a white solid. MS (ESI) m/z 555.3 [M+H]⁺.

¹H NMR (400 MHz, DMSO-d₆) δ=8.91 (d, J=7.7 Hz, 1H), 8.53 (d, J=8.5 Hz, 1H), 8.37 (d, J=7.7 Hz, 1H), 7.55 (br s, 1H), 7.34-7.26 (m, 2H), 7.06 (t, J=8.9 Hz, 2H), 6.48 (d, J=0.8 Hz, 1H), 5.08-4.96 (m, 1H), 4.75-4.63 (m, 1H), 4.34-4.22 (m, 1H), 3.11 3.06 (m, 2H), 3.06-2.92 (m, 2H), 2.44 (s, 3H), 2.30-2.18 (m, 2H), 1.85-1.67 (m, 3H), 1.60-1.37 (m, 5H), 0.94-0.89 (m, 3H), 0.87-0.83 (m, 3H)

Example 5: Synthesis of Benzyl ((2S)-1-((1-(((S)-1-cyano-2-((S)-2-oxopiperidin-3-yl)ethyl)amino)-3-(4,4-difluorocyclohexyl)-1-oxopropan-2-yl)amino)-3-(naphthalen-1-yl)-1-oxopropan-2-yl)carbamate (Compound 114)

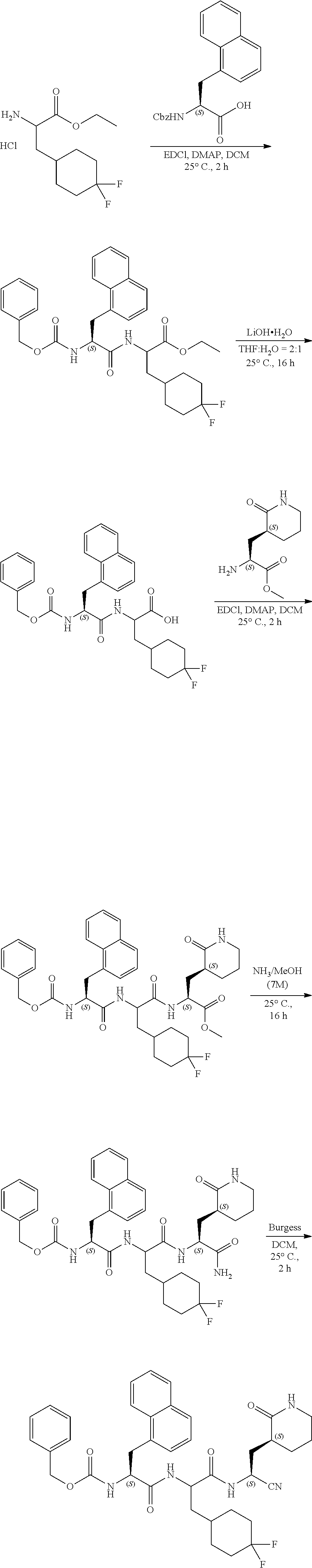

Step 1: ethyl 2-((S)-2-(((benzyloxy)carbonyl)amino)-3-(naphthalen-1-yl)propanamido)-3-(4,4-difluorocyclohexyl)propanoate

To a solution of (2S)-2-(benzyloxycarbonylamino)-3-(1-naphthyl)propanoic acid (1.29 g, 3.68 mmol, 1 eq) and ethyl 2-amino-3-(4,4-difluorocyclohexyl)propanoate (1.00 g, 3.68 mmol, 1 eq, HCl) in DCM (15 mL) was added EDCI (1.41 g, 7.36 mmol, 2 eq) and DMAP (1.35 g, 11.04 mmol, 3 eq). The mixture was stirred at 25° C. for 2 h. Upon completion, the reaction solution was quenched with H2O (20 mL) and extracted with DCM 60 mL (20 mL*3). The organic layers were combined, washed with citric acid (20 mL), sat. NaHCO₃aq (20 mL), brine (20 mL), dried over Na₂SO₄ and concentrated under reduced pressure. The residue was purified by column chromatography (SiO₂, Petroleum ether:Ethyl acetate=5:1 to 3:1) to give ethyl 2-[[(2S)-2-(benzyloxycarbonylamino)-3-(1-naphthyl)propanoyl]amino]-3-(4,4-difluorocyclohexyl)propanoate (1.6 g, 2.73 mmol, 74.20% yield, 96.7% purity) as a white solid. MS (ESI) m/z 567.2 [M+H]⁺.

Step 2: 2-((S)-2-(((benzyloxy)carbonyl)amino)-3-(naphthalen-1-yl)propanamido)-3-(4,4-difluorocyclohexyl)propanoic acid

To a solution of ethyl 2-[[(2S)-2-(benzyloxycarbonylamino)-3-(1-naphthyl)propanoyl]amino]-3-(4,4-difluorocyclohexyl)propanoate (1.60 g, 2.82 mmol, 1 eq) in THE (15 mL) and H2O (8 mL) was added LiOH.H₂O (355.48 mg, 8.47 mmol, 3 eq). The mixture was stirred at 25° C. for 16 h. Upon completion, to the reaction solution was added HCl (1 M) until pH of the solution was 2. Then the reaction solution was extracted with EtOAc 120 mL (40 mL*3). The organic layers were combined, dried over Na₂SO₄, and concentrated under reduced pressure to give 2-[[(2S)-2-(benzyloxycarbonylamino)-3-(1-naphthyl)propanoyl]amino]-3-(4,4-difluorocyclohexyl)propanoic acid (1.5 g, crude) as a white solid. MS (ESI) m/z 537.0 [M−H]⁺.

Step 3: methyl (2S)-2-[[(2S)-2-[[(2S)-2-(benzyloxycarbonylamino)-3-(1-naphthyl)propanoyl]amino]-3-(4,4-difluorocyclohexyl)propanoyl]amino]-3-[(3S)-2-oxo-3-piperidyl]propanoate

To a solution of (2S)-2-[[(2S)-2-(benzyloxycarbonylamino)-3-(1-naphthyl)propanoyl]amino]-3-(4,4-difluorocyclohexyl)propanoic acid (300 mg, 557.02 umol, 1 eq) and methyl (2S)-2-amino-3-[(3S)-2-oxo-3-piperidyl]propanoate (131.84 mg, 557.02 umol, 1 eq, HCl) in DCM (10 mL) was added EDCI (213.56 mg, 1.11 mmol, 2 eq) and DMAP (204.15 mg, 1.67 mmol, 3 eq). The mixture was stirred at 25° C. for 2 h. Upon completion, the reaction solution was quenched with H2O (20 mL), extracted with DCM 60 mL (20 mL*3). The organic layers were combined, washed with citric acid (20 mL), sat. NaHCO₃aq (20 mL), brine (20 mL), dried over Na₂SO₄ and concentrated under reduced pressure to give a residue. The residue was purified by column chromatography (SiO₂, DCM:MeOH=10:1) to give methyl (2S)-2-[[(2S)-2-[[(2S)-2-(benzyloxycarbonylamino)-3-(1-naphthyl)propanoyl]amino]-3-(4,4-difluorocyclohexyl)propanoyl]amino]-3-[(3S)-2-oxo-3-piperidyl]propanoate (280 mg, 314.65 umol, 56.49% yield, 81% purity) as a white solid. MS (ESI) m/z 721.3 [M+H]⁺.

Step 4: methyl (2S)-2-[[(2S)-2-[[(2S)-2-(benzyloxycarbonylamino)-3-(1-naphthyl)propanoyl]amino]-3-(4,4-difluorocyclohexyl)propanoyl]amino]-3-[(3S)-2-oxo-3-piperidyl]propanoate

A solution of methyl (2S)-2-[[(2S)-2-[[(2S)-2-(benzyloxycarbonylamino)-3-(1-naphthyl)propanoyl]amino]-3-(4,4-difluorocyclohexyl)propanoyl]amino]-3-[(3S)-2-oxo-3-piperidyl]propanoate (270 mg, 374.58 umol, 1 eq) in NH₃/MeOH (7 M, 5 mL, 93.44 eq) was stirred at 25° C. for 16 h. Upon completion, the reaction solution was concentrated under reduced pressure give benzyl N-[(1S)-2-[[(1S)-2-[[(1S)-2-amino-2-oxo-1-[[(3S)-2-oxo-3-piperidyl]methyl]ethyl]amino]-1-[(4,4-difluorocyclohexyl)methyl]-2-oxo-ethyl]amino]-1-(1-naphthylmethyl)-2-oxo-ethyl]carbamate (240 mg, 340.05 umol, 90.78% yield) as a white solid. MS (ESI) m/z 706.5 [M+H]⁺.

Step 5: benzyl N-[(1S)-2-[[2-[[(1S)-1-cyano-2-[(3S)-2-oxo-3-piperidyl]ethyl]amino]-1-[(4,4-difluorocyclohexyl)methyl]-2-oxo-ethyl]amino]-1-(1-naphthylmethyl)-2-oxo-ethyl]carbamate

To a solution of benzyl N-[(1S)-2-[[(1S)-2-[[(1S)-2-amino-2-oxo-1-[[(3S)-2-oxo-3-piperidyl]methyl]ethyl]amino]-1-[(4,4-difluorocyclohexyl)methyl]-2-oxo-ethyl]amino]-1-(1-naphthylmethyl)-2-oxo-ethyl]carbamate (240 mg, 340.05 umol, 1 eq) in DCM (5 mL) was added Burgess reagent (243.10 mg, 1.02 mmol, 3 eq). The mixture was stirred at 25° C. for 2 h. Upon completion, the reaction mixture was quenched by H2O (20 mL). The organic phase was separated, washed with DCM 60 mL (20 mL*3), dried over Na₂SO₄, filtered and concentrated under reduced pressure to give a residue. The residue was purified by prep-TLC (SiO₂, DCM:MeOH=10:1). The residue was further separated by SFC (REGIS(S, S)WHELK-O1(250 mm*25 mm, 10 um); mobile phase: [Neu-IPA]; B %: 50%-50%, 15 min) to give benzyl N-[(1S)-2-[[2-[[(1S)-1-cyano-2-[(3S)-2-oxo-3-piperidyl]ethyl]amino]-1-[(4,4-difluorocyclohexyl)methyl]-2-oxo-ethyl]amino]-1-(1-naphthylmethyl)-2-oxo-ethyl]carbamate (Isomer 1, 31 mg, 45.07 umol, 13.25% yield, 100% purity) and benzyl N [(1S)-2-[[2-[[(1S)-1-cyano-2-[(3S)-2-oxo-3-piperidyl]ethyl]amino]-1-[(4,4-difluorocyclohexyl)methyl]-2-oxo-ethyl]amino]-1-(1-naphthylmethyl)-2-oxo-ethyl]carbamate (Isomer 2) (28 mg, 39.12 umol, 11.51% yield, 96.1% purity) as white solids. MS (ESI) m/z 688.3 [M+H]⁺.

Isomer 1: ¹H NMR (400 MHz, DMSO-d₆) δ=8.85 (br d, J=7.9 Hz, 1H), 8.30 (br d, J=7.9 Hz, 1H), 8.18 (br d, J=8.3 Hz, 1H), 7.92 (br d, J=7.8 Hz, 1H), 7.80 (br d, J=7.4 Hz, 1H), 7.66-7.49 (m, 4H), 7.47-7.36 (m, 2H), 7.35-7.24 (m, 3H), 7.19 (brd, J=6.5 Hz, 2H), 5.10-4.98 (m, 1H), 4.91 (s, 2H), 4.52-4.28 (m, 2H), 3.52 (br dd, J=3.9, 14.2 Hz, 1H), 3.17 (br dd, J=10.6, 14.4 Hz, 1H), 3.10 (br s, 2H), 2.30-2.19 (m, 2H), 1.97 (br s, 2H), 1.85 (br s, 1H), 1.82-1.68 (m, 5H), 1.58 (br dd, J=6.9, 14.1 Hz, 4H), 1.49-1.34 (m, 2H), 1.28-1.13 (m, 2H).

Isomer 2: ¹H NMR (400 MHz, DMSO-d₆) δ=8.68 (br d, J=7.9 Hz, 1H), 8.34 (br d, J=8.1 Hz, 1H), 8.19 (br d, J=8.5 Hz, 1H), 7.92 (br d, J=7.8 Hz, 1H), 7.80 (br d, J=7.6 Hz, 1H), 7.73 (br d, J=7.5 Hz, 1H), 7.63-7.47 (m, 3H), 7.41 (q, J=7.1 Hz, 2H), 7.37 7.15 (m, 5H), 5.12-5.02 (m, 1H), 4.98 (s, 2H), 4.46 (q, J=7.6 Hz, 1H), 4.38-4.16 (m, 1H), 3.47 (br dd, J=6.4, 13.7 Hz, 1H), 3.24 (br dd, J=8.5, 13.8 Hz, 1H), 3.09 (br s, 2H), 2.26 2.12 (m, 2H), 1.95-1.69 (m, 5H), 1.65-1.35 (m, 7H), 1.23 (br s, 1H), 1.15-0.93 (m, 3H).

Example 6: Synthesis of N—((S)-1-(((S)-1-(((S)-1-cyano-2-((S)-2-oxopiperidin-3-yl)ethyl)amino)-3-cyclopropyl-1-oxopropan-2-yl)amino)-3-(naphthalen-1-yl)-1-oxopropan-2-yl)pyrazine-2-carboxamide (Compound 115)

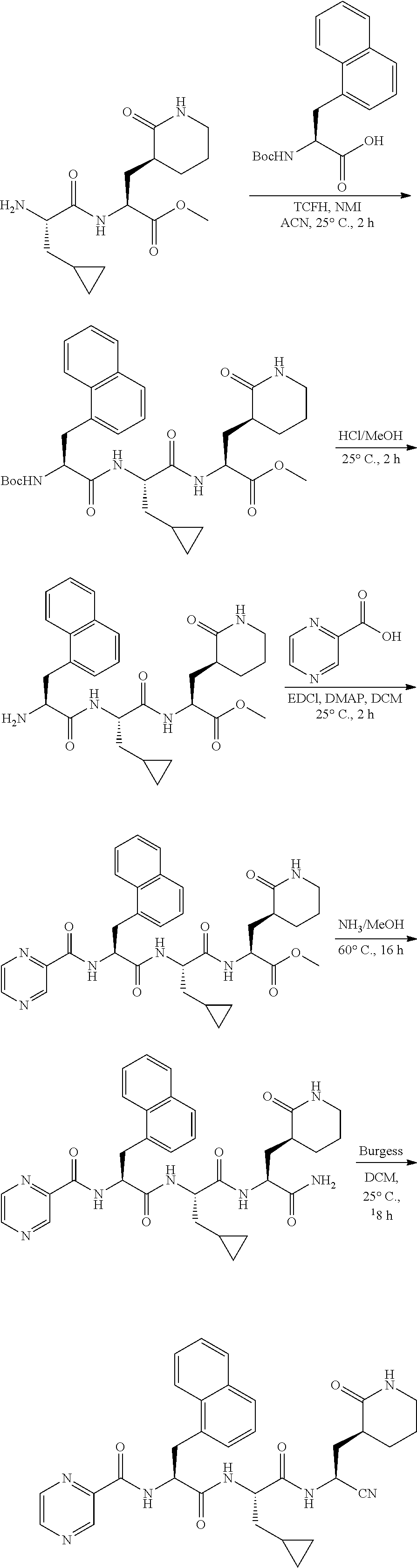

Step 1: methyl (2S)-2-[[(2S)-2-[[(2S)-2-(tert-butoxycarbonylamino)-3-(1-naphthyl)propanoyl]amino]-3-cyclopropyl-propanoyl]amino]-3-[(3S)-2-oxo-3-piperidyl]propanoate

To a solution of methyl (2S)-2-[[(2S)-2-amino-3-cyclopropyl-propanoyl]amino]-3-[(3S)-2-oxo-3-piperidyl]propanoate (1.5 g, 4.82 mmol, 1 eq) and (S)-2-((tert-butoxycarbonyl)amino)-3-(naphthalen-1-yl)propanoic acid (1.52 g, 4.82 mmol, 1 eq) in ACN (10 mL) was added 1-methylimidazole (1.19 g, 14.45 mmol, 1.15 mL, 3 eq) and [chloro(dimethylamino)methylene]-dimethyl-ammonium hexafluorophosphate (1.35 g, 4.82 mmol, 1 eq). The mixture was stirred at 25° C. for 2 h. Upon completion, the reaction mixture was concentrated under reduced pressure to remove ACN. The residue was diluted with H2O 10 mL and extracted with DCM 30 mL (10 mL*3). The combined organic layers were washed with brine (10 mL), dried over Na₂SO₄, filtered and concentrated under reduced pressure to give a residue. The residue was purified by column chromatography (SiO₂, Petroleum ether/Ethyl acetate=5/1 to 0/1) to give methyl (2S)-2-[[(2S)-2-[[(2S)-2-(tert-butoxycarbonylamino)-3-(1-naphthyl)propanoyl]amino]-3-cyclopropyl-propanoyl]amino]-3-[(3S)-2-oxo-3-piperidyl]propanoate (2.5 g, 2.87 mmol, 59.68% yield, 70% purity) as a light yellow solid. MS (ESI) m/z 609.3 [M+H]⁺.

Step 2: methyl (2S)-2-[[(2S)-2-[[(2S)-2-amino-3-(1-naphthyl)propanoyl]amino]-3-cyclopropyl-propanoyl]amino]-3-[(3S)-2-oxo-3-piperidyl]propanoate

To a solution of methyl (2S)-2-[[(2S)-2-[[(2S)-2-(tert-butoxycarbonylamino)-3-(1-naphthyl)propanoyl]amino]-3-cyclopropyl-propanoyl]amino]-3-[(3S)-2-oxo-3-piperidyl]propanoate (1 g, 1.64 mmol, 1 eq) was added HCl/MeOH (15 mL). The mixture was stirred at 25° C. for 2 hours. Upon completion, the reaction mixture was concentrated under reduced pressure to give methyl (2S)-2-[[(2S)-2-[[(2S)-2-amino-3-(1-naphthyl)propanoyl]amino]-3-cyclopropyl-propanoyl]amino]-3-[(3S)-2-oxo-3-piperidyl]propanoate (800 mg, 943.75 umol, 57.45% yield, 60% purity) as a colorless oil. MS (ESI) m/z 509.3 [M+H]⁺.

Step 3: methyl (2S)-2-[[(2S)-3-cyclopropyl-2-[[(2S)-3-(1-naphthyl)-2-(pyrazine-2-carbonylamino)propanoyl]amino]propanoyl]amino]-3-[(3S)-2-oxo-3-piperidyl]propanoate

To a solution of methyl (2S)-2-[[(2S)-2-[[(2S)-2-amino-3-(1-naphthyl)propanoyl]amino]-3-cyclopropyl-propanoyl]amino]-3-[(3S)-2-oxo-3-piperidyl]propanoate (400 mg, 786.46 umol, 1 eq) and pyrazine-2-carboxylic acid (97.60 mg, 786.46 umol, 1 eq) in DCM (5 mL) was added EDCI (150.77 mg, 786.46 umol, 1 eq) and DMAP (96.08 mg, 786.46 umol, 1 eq). The mixture was stirred at 25° C. for 2 hours. Upon completion, the reaction mixture was quenched by addition H2O (10 mL) at 25° C., and then extracted with ethyl acetate 15 mL (5 mL*3). The combined organic layers were washed with brine (10 mL), dried over Na₂SO₄, filtered and concentrated under reduced pressure to give a residue. The residue was purified by prep-TLC (SiO₂, PE: ethyl acetate=0:1) to give methyl (2S)-2-[[(2S)-3-cyclopropyl-2-[[(2S)-3-(1-naphthyl)-2-(pyrazine-2-carbonylamino)propanoyl]amino]propanoyl]amino]-3-[(3S)-2-oxo-3-piperidyl]propanoate (60 mg, 97.61 umol, 12.41% yield) as a white solid. MS (ESI) m/z 615.3 [M+H]⁺.

Step 4: N-[(1S)-2-[[(1S)-2-[[(1S)-2-amino-2-oxo-1-[[(3S)-2-oxo-3-piperidyl]methyl]ethyl]amino]-1-(cyclopropylmethyl)-2-oxo-ethyl]amino]-1-(1-naphthylmethyl)-2-oxo-ethyl]pyrazine-2-carboxamide

To methyl (2S)-2-[[(2S)-3-cyclopropyl-2-[[(2S)-3-(1-naphthyl)-2-(pyrazine-2-carbonylamino)propanoyl]amino]propanoyl]amino]-3-[(3S)-2-oxo-3-piperidyl]propanoate (60 mg, 97.61 umol, 1 eq) was added ammonia (7 M, 13.94 uL, 1 eq) in MeOH. The mixture was stirred at 60° C. for 16 hours. Upon completion, the reaction mixture was concentrated under reduced pressure to remove MeOH to give N-[(1S)-2-[[(1S)-2-[[(1S)-2-amino-2-oxo-1-[[(3S)-2-oxo-3-piperidyl]methyl]ethyl]amino]-1-(cyclopropylmethyl)-2-oxo-ethyl]amino]-1-(1-naphthylmethyl)-2-oxo-ethyl]pyrazine-2-carboxamide (30 mg, 44.52 umol, 45.61% yield, 89% purity) as a white solid. MS (ESI) m/z 600.4 [M+H]⁺.

Step 5: N—((S)-1-(((S)-1-(((S)-1-cyano-2-((S)-2-oxopiperidin-3-yl)ethyl)amino)-3-cyclopropyl-1-oxopropan-2-yl)amino)-3-(naphthalen-1-yl)-1-oxopropan-2-yl)pyrazine-2-carboxamide

To a solution of N-[(1S)-2-[[(1S)-2-[[(1S)-2-amino-2-oxo-1-[[(3S)-2-oxo-3-piperidyl]methyl]ethyl]amino]-1-(cyclopropylmethyl)-2-oxo-ethyl]amino]-1-(1-naphthylmethyl)-2-oxo-ethyl]pyrazine-2-carboxamide (30 mg, 50.03 umol, 1 eq) in DCM (1 mL) was added Burgess reagent (35.76 mg, 150.08 umol, 3 eq). The mixture was stirred at 25° C. for 18 hours. Upon completion, the reaction mixture was quenched with water (0.5 mL). The residue was purified by prep-HPLC (column: Phenomenex Luna C18 75*30 mm 3 um; mobile phase: [water(FA)-ACN]; B %: 40%-70%, 8 min) to give N—((S)-1-(((S)-1-(((S)-1-cyano-2-((S)-2-oxopiperidin-3-yl)ethyl)amino)-3-cyclopropyl-1-oxopropan-2-yl)amino)-3-(naphthalen-1-yl)-1-oxopropan-2-yl)pyrazine-2-carboxamide (5 mg, 8.60 umol 17.18% yield, purity, 99.08%) as a white solid. MS (ESI) m/z 582.2 [M+H]⁺.

¹H NMR (400 MHz, MeOD-d4) δ=9.09 (d, J=1.1 Hz, 1H), 8.74 (d, J=2.4 Hz, 1H), 8.61 (d, Jβ0.8 Hz, 1H), 8.25 (d, J=8.3 Hz, 1H), 7.83 (d, J=7.9 Hz, 1H), 7.73 (d, J=8.0 Hz, 1H), 7.54-7.39 (m, 3H), 7.37-7.26 (m, 1H), 5.15-5.01 (m, 2H), 4.33 (t, J=7.2 Hz, 1H), 3.79 (dd, J=5.8, 14.2 Hz, 1H), 3.58-3.46 (m, 1H), 3.28-3.21 (m, 2H), 2.51-2.36 (m, 2H), 2.06-1.97 (m, 1H), 1.92 (td, Jβ0.8, 13.3 Hz, 1H), 1.87-1.78 (m, 1H), 1.74 (td, Jβ0.8, 13.7 Hz, 2H), 1.62-1.46 (m, 2H), 0.77-0.66 (m, 1H), 0.46 (d, J=8.0 Hz, 2H), 0.16-0.04 (m, 2H)

Example 7: Synthesis of N—((S)-1-(((S)-1-(((S)-1-cyano-2-((S)-2-oxopiperidin-3-yl)ethyl)amino)-3-cyclopropyl-1-oxopropan-2-yl)amino)-3-(naphthalen-1-yl)-1-oxopropan-2-yl)-5-methylisoxazole-3-carboxamide (Compound 111)

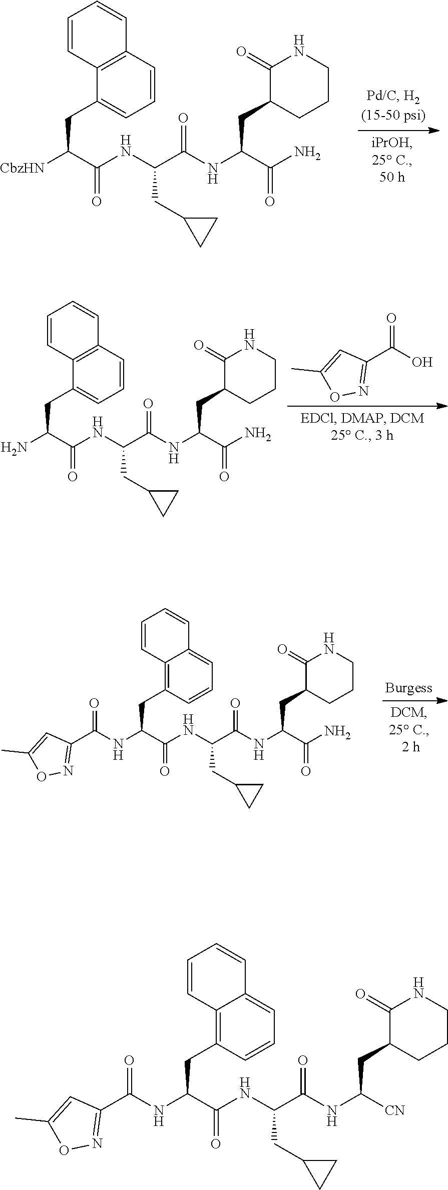

Step 1: (S)-2-amino-N—((S)-1-(((S)-1-amino-1-oxo-3-((S)-2-oxopiperidin-3-yl)propan-2-yl)amino)-3-cyclopropyl-1-oxopropan-2-yl)-3-(naphthalen-1-yl)propanamide

To a solution of benzyl ((S)-1-(((S)-1-(((S)-1-amino-1-oxo-3-((S)-2-oxopiperidin-3-yl)propan-2-yl)amino)-3-cyclopropyl-1-oxopropan-2-yl)amino)-3-(naphthalen-1-yl)-1-oxopropan-2-yl)carbamate (320.00 mg, 509.77 umol, 1.0 eq) in i-PrOH (30 mL) was added Pd/C (10%, 100.00 mg, 509.77 umol, 1.0 eq), the solution was stirred at 25° C. under H2 (15 psi) atmosphere for 16 h. Then the solution was filtered through Celite, washed with DCM/EtOAc/MeOH four times, purged with H2 for 3 times, added Pd/C (10%, 100.00 mg, 509.77 umol, 1.0 eq) and ran for another 20 h. LCMS showed that the ratio of desired product to starting material was 3:1. The reaction was kept running under H2 (50 psi) atmosphere at 25° C. for 14 h. Upon completion, the reaction mixture was filtered through Celite, washed with MeOH, concentrated under reduced pressure to give (S)-2-amino-N—((S) 1-(((S)-1-amino-1-oxo-3-((S)-2-oxopiperidin-3-yl)propan-2-yl)amino)-3-cyclopropyl-1-oxopropan-2-yl)-3-(naphthalen-1-yl)propanamide (240 mg, crude) as a white solid. MS (ESI) m/z 494.2 [M+H]⁺.

Step 2: N—((S)-1-(((S)-1-(((S)-1-amino-1-oxo-3-((S)-2-oxopiperidin-3-yl)propan-2-yl)amino)-3-cyclopropyl-1-oxopropan-2-yl)amino)-3-(naphthalen-1-yl)-1-oxopropan-2-yl)-5-methylisoxazole-3-carboxamide

To a solution of (S)-2-amino-N—((S)-1-(((S)-1-amino-1-oxo-3-((S)-2-oxopiperidin-3-yl)propan-2-yl)amino)-3-cyclopropyl-1-oxopropan-2-yl)-3-(naphthalen-1-yl)propanamide (110.00 mg, 222.85 umol, 1.0 eq) in DCM (5 mL) was added DMAP (81.68 mg, 668.55 umol, 3.0 eq), 5-methylisoxazole-3-carboxylic acid (28.32 mg, 222.85 umol, 1 eq) and EDCI (85.44 mg, 445.70 umol, 2.0 eq), the solution was stirred at 25° C. for 3 h. Upon completion, the reaction mixture was diluted with water (10 mL) and extracted with DCM (10 mL*3). The combined organic layers were combined, washed with citric acid, NaHCO₃aq., brine, dried over Na₂SO₄, filtered and concentrated under reduced pressure to give a residue. The residue was purified by column chromatography (SiO₂, DCM:MeOH=10:1) to give N—((S)-1-(((S)-1-(((S)-1-amino-1-oxo-3-((S)-2-oxopiperidin-3-yl)propan-2-yl)amino)-3-cyclopropyl-1-oxopropan-2-yl)amino)-3-(naphthalen-1-yl)-1-oxopropan-2-yl)-5-methylisoxazole-3-carboxamide (0.1 g, 165.93 umol, 74.46% yield) as a white solid. MS (ESI) m/z 603.3 [M+H]⁺.

Step 3: N—((S)-1-(((S)-1-(((S)-1-cyano-2-((S)-2-oxopiperidin-3-yl)ethyl)amino)-3-cyclopropyl-1-oxopropan-2-yl)amino)-3-(naphthalen-1-yl)-1-oxopropan-2-yl)-5-methylisoxazole-3-carboxamide

To a solution of N—((S)-1-(((S)-1-(((S)-1-amino-1-oxo-3-((S)-2-oxopiperidin-3-yl)propan-2-yl)amino)-3-cyclopropyl-1-oxopropan-2-yl)amino)-3-(naphthalen-1-yl)-1-oxopropan-2-yl)-5-methylisoxazole-3-carboxamide (90.00 mg, 149.33 umol, 1.0 eq) in DCM (1 mL) was added Burgess reagent (106.76 mg, 448.00 umol, 3.0 eq), and the solution was stirred at 25° C. for 1 h. Upon completion, the reaction mixture was diluted with water (0.5 mL) to give a residue. The residue was purified by prep-HPLC (column: Phenomenex Luna C18 200*40 mm*10 um; mobile phase: [water(FA)-ACN]; B %: 30%-70%, 8 min) to give N—((S)-1-(((S)-1-(((S)-1-cyano-2-((S)-2-oxopiperidin-3-yl)ethyl)amino)-3-cyclopropyl-1-oxopropan-2-yl)amino)-3-(naphthalen-1-yl)-1-oxopropan-2-yl)-5-methylisoxazole-3-carboxamide (8.00 mg, 13.68 umol, 9.16% yield, 100% purity) as a white solid. MS (ESI) m/z 585.2 [M+H]⁺.

¹H NMR (400 MHz, DMSO-d₆) δ=8.86 (d, J=7.9 Hz, 1H), 8.73-8.65 (m, 1H), 8.41-8.32 (m, 1H), 8.20-8.12 (m, 1H), 7.91-7.82 (m, 1H), 7.78-7.69 (m, 1H), 7.56-7.44 (m, 3H), 7.41-7.28 (m, 2H), 6.42-6.37 (m, 1H), 5.07-4.97 (m, 1H), 4.91-4.81 (m, 1H), 4.35-4.25 (m, 1H), 3.64-3.56 (m, 1H), 3.40-3.34 (m, 1H), 3.10-3.02 (m, 2H), 2.39 (s, 3H), 2.25-2.17 (m, 2H), 1.87-1.79 (m, 1H), 1.79-1.71 (m, 1H), 1.68-1.59 (m, 2H), 1.59 1.50 (m, 1H), 1.44-1.36 (m, 2H), 0.75-0.66 (m, 1H), 0.44-0.32 (m, 2H), 0.15-0.01 (m, 2H).

Example 8: Synthesis of Benzyl((S)-1-(((S)-1-(((S)-1-cyano-2-((S)-2-oxopiperidin-3-yl)ethyl)amino)-3-cyclopropyl-1-oxopropan-2-yl)amino)-3-(naphthalen-1-yl)-1-oxopropan-2-yl)carbamate (Compound 116)

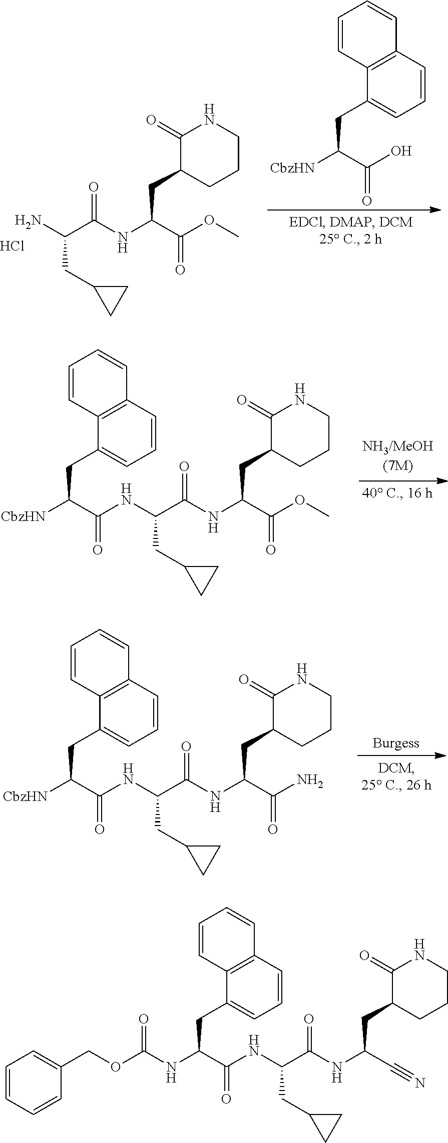

Step 1: (5S,8S,11S)-methyl 8-(cyclopropylmethyl)-5-(naphthalen-1-ylmethyl)-3,6,9-trioxo-11-(((S)-2-oxopiperidin-3-yl)methyl)-1-phenyl-2-oxa-4,7,10-triazadodecan-12-oate

To a solution of (S)-methyl 2-((S)-2-amino-3-cyclopropylpropanamido)-3-((S)-2-oxopiperidin-3-yl)propanoate hydrochloride (534.74 mg, 1.72 mmol, 1.2 eq, HCl) in DCM (5 mL) was added DMAP (524.51 mg, 4.29 mmol, 3.0 eq), (S)-2-(((benzyloxy)carbonyl)amino)-3-(naphthalen-1-yl)propanoic acid (500.00 mg, 1.43 mol, 1.0 eq) and EDCI (548.69 mg, 2.86 mmol, 2.0 eq), the solution was stirred at 25° C. for 2 h. Upon completion, the reaction mixture was diluted with water (5 mL) and extracted with ethyl acetate (5 mL*4). The combined organic layers were dried over Na₂SO₄, filtered and concentrated under reduced pressure to give a residue. The residue was purified by column chromatography (SiO₂, DCM:MeOH=10:1) to give methyl (2S)-2-[[(2S)-2-[[(2S)-2-(benzyloxycarbonylamino)-3-(1-naphthyl)propanoyl]amino]-3-cyclopropyl-propanoyl]amino]-3-[(3S)-2-oxo-3-piperidyl]propanoate (521 mg, 810.59 umol, 56.64% yield, 90% purity) as a white solid. MS (ESI) m/z 643.3 [M+H]⁺.

Step 2: benzyl ((S)-1-(((S)-1-(((S)-1-amino-1-oxo-3-((S)-2-oxopiperidin-3-yl)propan-2-yl)amino)-3-cyclopropyl-1-oxopropan-2-yl)amino)-3-(naphthalen-1-yl)-1-oxopropan-2-yl)carbamate

To a solution of (5S,8S,11S)-methyl 8-(cyclopropylmethyl)-5-(naphthalen-1-ylmethyl)-3,6,9-trioxo-11-(((S)-2-oxopiperidin-3-yl)methyl)-1-phenyl-2-oxa-4,7,10-triazadodecan-12-oate (511.00 mg, 795.03 umol, 1 eq) in NH₃/MeOH (5 mL) was stirred at 40° C. for 16 h. Upon completion, the reaction mixture was concentrated under reduced pressure to give benzyl ((S)-1-(((S)-1-(((S)-1-amino-1-oxo-3-((S)-2-oxopiperidin-3-yl)propan-2-yl)amino)-3-cyclopropyl-1-oxopropan-2-yl)amino)-3-(naphthalen-1-yl)-1-oxopropan-2-yl)carbamate (420 mg, crude) as a white solid.

Step 3: benzyl ((S)-1-(((S)-1-(((S)-1-cyano-2-((S)-2-oxopiperidin-3-yl)ethyl)amino)-3-cyclopropyl-1-oxopropan-2-yl)amino)-3-(naphthalen-1-yl)-1-oxopropan-2-yl)carbamate

A mixture of benzyl ((S)-1-(((S)-1-(((S)-1-amino-1-oxo-3-((S)-2-oxopiperidin-3-yl)propan-2-yl)amino)-3-cyclopropyl-1-oxopropan-2-yl)amino)-3-(naphthalen-1-yl)-1-oxopropan-2-yl)carbamate (80.00 mg, 127.44 umol, 1.0 eq) was added Burgess reagent (182.22 mg, 764.66 umol, 6.0 eq), and the solution was stirred at 25° C. for 26 h. Upon completion, the reaction mixture was diluted with water (10 mL) and extracted with ethyl acetate (5 mL*3). The combined organic layers were dried over Na₂SO₄, filtered and concentrated under reduced pressure to give a residue. The residue was purified by prep-HPLC (column: Phenomenex Luna C18 75*30 mm*3 um; mobile phase: [water(FA)-ACN]; B %: 40%-70%, 8 min) to give benzyl ((S)-1-(((S)-1-(((S)-1-cyano-2-((S)-2-oxopiperidin-3-yl)ethyl)amino)-3-cyclopropyl-1-oxopropan-2-yl)amino)-3-(naphthalen-1-yl)-1-oxopropan-2-yl)carbamate (30.00 mg, 49.20 umol, 38.61% yield, 100% purity) as a white solid. MS (ESI) m/z 610.2 [M+H]⁺.

¹H NMR (400 MHz, MeOD-d₄) δ=8.18 (br d, J=7.7 Hz, 1H), 7.87 (br d, J=7.5 Hz, 1H), 7.76 (br s, 1H), 7.59-7.43 (m, 2H), 7.41-6.90 (m, 7H), 5.15-5.03 (m, 1H), 4.98 (s, 2H), 4.58 (br s, 2H), 4.31 (br t, J=7.0 Hz, 1H), 3.76-3.61 (m, 1H), 3.25 (br s, 2H), 2.41 (br d, J=9.8 Hz, 2H), 1.99 (br s, 1H), 1.95-1.86 (m, 1H), 1.81 (br s, 1H), 1.71 (br d, J=6.3 Hz, 2H), 1.51-1.30 (br s, 2H), 0.69 (br s, 1H), 0.46 (br d, J=7.7 Hz, 2H), 0.10 (br s, 2H)

Example 9: Synthesis of ((S)-1-(((S)-3-cyclopropyl-1-oxo-1-(((S)-1-oxo-3-((S)-2-oxopyrrolidin-3-yl)propan-2-yl)amino)propan-2-yl)amino)-3-(naphthalen-1-yl)-1-oxopropan-2-yl)carbamate (Compound 117)

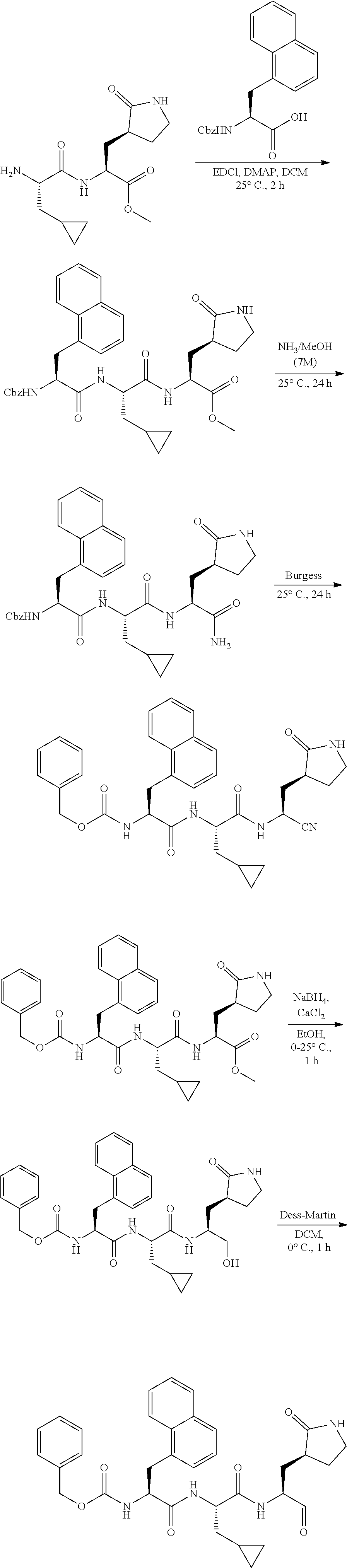

Step 1: (5S,8S,11S)-methyl 8-(cyclopropylmethyl)-5-(naphthalen-1-ylmethyl)-3,6,9-trioxo-11-(((S)-2-oxopyrrolidin-3-yl)methyl)-1 phenyl-2-oxa-4,7,10-triazadodecan-12-oate

To a solution of (2S)-2-(benzyloxycarbonylamino)-3-(1-naphthyl)propanoic acid (500 mg, 1.43 mmol, 1 eq), methyl (2S)-2-[[(2S)-2-amino-3-cyclopropyl-propanoyl]amino]-3-[(3S)-2-oxopyrrolidin-3-yl]propanoate (573.26 mg, 1.72 mmol, 1.2 eq, HCl), DMAP (437.09 mg, 3.58 mmol, 2.5 eq) in DCM (5 mL) was added EDCI (548.69 mg, 2.86 mmol, 2 eq). The mixture was stirred at 25° C. for 2 h. Upon completion, the reaction mixture was poured into H₂O 30 mL at 25° C., and then extracted with DCM (30 mL*3). The combined organic layers were washed with brine (30 mL*2), dried over Na₂SO4, filtered and concentrated under reduced pressure to give a residue. The residue was purified by column chromatography (SiO₂, DCM/MeOH=100/0 to 80/20) to give methyl (2S)-2-[[(2S)-2-[[(2S)-2-(benzyloxycarbonylamino)-3-(1-naphthyl)propanoyl]amino]-3-cyclopropyl-propanoyl]amino]-3-[(3S)-2-oxopyrrolidin-3-yl]propanoate (500 mg, 776.19 umol, 54.24% yield, 97.6% purity) as a white solid. MS (ESI) m/z 629.3 [M+H]⁺.

Step 2: benzyl ((S)-1-(((S)-1-(((S)-1-amino-1-oxo-3-((S)-2-oxopyrrolidin-3-yl)propan-2-yl)amino)-3-cyclopropyl-1-oxopropan-2-yl)amino)-3-(naphthalen-1-yl)-1-oxopropan-2-yl)carbamate

To a solution of methyl (2S)-2-[[(2S)-2-[[(2S)-2-(benzyloxycarbonylamino)-3-(1-naphthyl)propanoyl]amino]-3-cyclopropyl-propanoyl]amino]-3-[(3S)-2-oxopyrrolidin-3-yl]propanoate (130 mg, 206.77 umol, 1 eq) in NH₃/MeOH (7 M, 5 mL, 169.27 eq) was stirred at 25° C. for 24 h. Upon completion, the reaction mixture was concentrated under reduced pressure to remove solvent. The residue was purified by prep-HPLC (column: Phenomenex C18 80*40 mm*3 um; mobile phase: [water(NH₄HCO₃)-ACN]; B %: 45%-75%, 8 min) to give benzyl N-[(1S)-2-[[(1S)-2-[[(1S)-2-amino-2-oxo-1-[[(3S)-2-oxopyrrolidin-3-yl]methyl]ethyl]amino]-1-(cyclopropylmethyl)-2-oxo-ethyl]amino]-1-(1-naphthylmethyl)-2-oxo-ethyl]carbamate (85 mg, 138.50 umol, 66.98% yield) as a white solid. MS (ESI) m/z 614.3 [M+H]⁺.

Step 3: benzyl ((S)-1-(((S)-1-(((S)-1-cyano-2-((S)-2-oxopyrrolidin-3-yl)ethyl)amino)-3-cyclopropyl-1-oxopropan-2-yl)amino)-3-(naphthalen-1-yl)-1-oxopropan-2-yl)carbamate

To a solution of benzyl N-[(1S)-2-[[(1S)-2-[[(1S)-2-amino-2-oxo-1-[[(3S)-2-oxopyrrolidin-3-yl]methyl]ethyl]amino]-1-(cyclopropylmethyl)-2-oxo-ethyl]amino]-1-(1-naphthylmethyl)-2-oxo-ethyl]carbamate (80 mg, 130.36 umol, 1 eq) in DCM (3 mL) was added Burgess reagent (155.32 mg, 651.78 umol, 5 eq). The mixture was stirred at 30° C. for 24 h. Upon completion, the reaction mixture was concentrated under reduced pressure to remove solvent. The residue was purified by prep-HPLC (column: Waters Xbridge Prep OBD C18 150*40 mm*10 um; mobile phase: [water(NH₄HCO₃)-ACN]; B %: 35%-65%, 8 min) to give benzyl N-[(1S)-2-[[(1S)-2-[[(1S)-1-cyano-2-[(3S)-2-oxopyrrolidin-3-yl]ethyl]amino]-1-(cyclopropylmethyl)-2-oxo-ethyl]amino]-1-(1-naphthylmethyl)-2-oxo-ethyl]carbamate (5 mg, 8.39 umol, 7.14% yield) as a white solid. MS (ESI) m/z 596.2 [M+H]⁺.

¹H NMR (400 MHz, METHANOL-d4) δ=8.18 (d, J=8.3 Hz, 1H), 7.86 (d, J=7.9 Hz, 1H), 7.80-7.72 (m, 1H), 7.56-7.46 (m, 2H), 7.40-7.18 (m, 7H), 5.05-4.95 (m, 3H), 4.57 (d, J=5.8, 8.8 Hz, 1H), 4.30 (t, J=7.1 Hz, 1H), 3.67 (d, J=5.6, 14.2 Hz, 1H), 3.30-3.22 (m, 3H), 2.66-2.49 (m, 1H), 2.34-2.25 (m, 2H), 1.93-1.69 (m, 3H), 1.51 (d, J=7.1 Hz, 1H), 0.69 (d, J=1.8 Hz, 1H), 0.46 (d, J=8.3 Hz, 2H), 0.10 (d, J=4.9, 6.7 Hz, 2H).

Step 4: benzyl ((S)-1-(((S)-3-cyclopropyl-1-(((S)-1-hydroxy-3-((S)-2-oxopyrrolidin-3-yl)propan-2-yl)amino)-1-oxopropan-2-yl)amino)-3-(naphthalen-1-yl)-1-oxopropan-2-yl)carbamate

To a solution of methyl (2S)-2-[[(2S)-2-[[(2S)-2-(benzyloxycarbonylamino)-3-(1-naphthyl)propanoyl]amino]-3-cyclopropyl-propanoyl]amino]-3-[(3S)-2-oxopyrrolidin-3-yl]propanoate (360 mg, 572.60 umol, 1 eq), CaCl₂) (190.65 mg, 1.72 mmol, 3 eq) in EtOH (5 mL) was added NaBH4 (216.63 mg, 5.73 mmol, 10 eq) at 0° C. The mixture was stirred at 25° C. for 1 h. Upon completion, the reaction mixture was poured into NH₄C1(a.q) 30 mL at 20° C., and then extracted with EtOAc (30 mL*3). The combined organic layers were washed with brine (30 mL*2), dried over Na₂SO4, filtered and concentrated under reduced pressure to give a residue. The residue was purified by column chromatography (SiO₂, Petroleum ether/Ethyl acetate=100/0 to 80/20) to give benzyl N-[(1S)-2-[[(1S)-1-(cyclopropylmethyl)-2-[[(1S)-1-(hydroxymethyl)-2-[(3S)-2-oxopyrrolidin-3-yl]ethyl]amino]-2-oxo-ethyl]amino]-1-(1-naphthylmethyl)-2-oxo-ethyl]carbamate (305 mg, 507.74 umol, 88.67% yield) as a white solid. MS (ESI) m/z 601.3 [M+H]⁺.

¹H NMR (400 MHz, DMSO-d₆)(DMSO) δ=8.18 (d, J=8.1 Hz, 1H), 8.09 (d, J=8.0 Hz, 1H), 7.92 (d, J=7.5 Hz, 1H), 7.85-7.72 (m, 2H), 7.67 (d, J=8.7 Hz, 1H), 7.61 7.49 (m, 3H), 7.47-7.36 (m, 2H), 7.33-7.26 (m, 2H), 7.23-7.12 (m, 2H), 4.90 (s, 2H), 4.68 (t, J=5.4 Hz, 1H), 4.54-4.28 (m, 2H), 3.88-3.73 (m, 1H), 3.63-3.49 (m, 1H), 3.42-3.37 (m, 1H), 3.25 (d, J=5.4, 10.9 Hz, 1H), 3.19-3.01 (m, 3H), 2.30-2.08 (m, 2H), 1.79 (s, 1H), 1.66-1.33 (m, 4H), 0.80-0.64 (m, 1H), 0.47-0.30 (m, 2H), 0.18-0.01 (m, 2H)

¹H NMR (400 MHz, DMSO-d₆)(DMSO+D₂O) δ=8.16 (d, J=8.2 Hz, 1H), 8.07 (d, J=7.7 Hz, 1H), 7.91 (d, J=7.4 Hz, 1H), 7.78 (t, J=7.7 Hz, 2H), 7.64 (d, J=8.7 Hz, 1H), 7.58-7.49 (m, 2H), 7.46-7.35 (m, 2H), 7.30-7.25 (m, 2H), 7.19-7.12 (m, 2H), 6.93 6.78 (m, 1H), 4.88 (s, 2H), 4.61-4.19 (m, 2H), 3.86-3.72 (m, 1H), 3.54 (d, J=3.8, 14.2 Hz, 1H), 3.41-3.30 (m, 1H), 3.24 (d, J=6.6, 10.6 Hz, 1H), 3.19-2.99 (m, 3H), 2.24 (d, J=8.9 Hz, 1H), 2.18-2.08 (m, 1H), 1.83-1.71 (m, 1H), 1.66-1.50 (m, 2H), 1.50-1.33 (m, 2H), 0.69 (d, J=6.6 Hz, 1H), 0.45-0.27 (m, 2H), 0.07 (d, J=4.6, 7.7, 12.0 Hz, 2H).

Step 5: benzyl ((S)-1-(((S)-3-cyclopropyl-1-oxo-1-(((S)-1-oxo-3-((S)-2-oxopyrrolidin-3-yl)propan-2-yl)amino)propan-2-yl)amino)-3-(naphthalen-1-yl)-1-oxopropan-2-yl)carbamate

To a solution of benzyl N-[(1S)-2-[[(1S)-1-(cyclopropylmethyl)-2-[[(1S)-1-(hydroxymethyl)-2-[(3S)-2-oxopyrrolidin-3-yl]ethyl]amino]-2-oxo-ethyl]amino]-1-(1-naphthylmethyl)-2-oxo-ethyl]carbamate (140 mg, 233.06 umol, 1 eq) in DCM (3 mL) was added Dess-Martin (197.70 mg, 466.12 umol, 144.31 uL, 2 eq) at 0° C. Then, the mixture was stirred at 25° C. for 1.5 h. Upon completion, the reaction mixture was poured into H2O 15 mL at 25° C., and then extracted with DCM (25 mL*3). The combined organic layers were washed with brine (20 mL*2), dried over NaSO4, filtered and concentrated under reduced pressure to give a residue. The residue was purified by prep-HPLC (column: Waters Xbridge Prep OBD C18 150*40 mm*10 um; mobile phase: [water(NH₄HCO₃)-ACN]; B %: 40%-70%, 8 min) to give benzyl N-[(1S)-2-[[(1S)-1-(cyclopropylmethyl)-2-[[(1S)-1-formyl-2-[(3S)-2-oxopyrrolidin-3-yl]ethyl]amino]-2-oxo-ethyl]amino]-1-(1-naphthylmethyl)-2-oxo-ethyl]carbamate (30 mg, 49.16 umol, 21.09% yield, 98.1% purity) as a white solid. MS (ESI) m/z 599.1 [M+H]⁺.

¹H NMR (400 MHz, DMSO-d₆) δ=9.43 (s, 1H), 8.53 (d, J=7.1 Hz, 1H), 8.19 (d, J=7.0 Hz, 2H), 7.92 (d, J=7.8 Hz, 1H), 7.80 (d, J=7.8 Hz, 1H), 7.70-7.35 (m, 6H), 7.33-7.25 (m, 2H), 7.18 (d, J=6.5 Hz, 2H), 5.72 (brs, 1H), 4.90 (s, 2H), 4.74-4.14 (m, 3H), 3.83-3.47 (m, 2H), 3.21-2.93 (m, 3H), 2.16 (s, 2H), 1.91 (s, 1H), 1.73-1.35 (m, 4H), 0.76 (d, J=4.6 Hz, 1H), 0.39 (d, J=2.9 Hz, 2H), 0.18-0.00 (m, 2H).

¹H NMR (400 MHz, DMSO-d₆)(273+80 K) δ=9.45 (s, 1H), 8.30-8.08 (m, 2H), 7.90 (d, J=7.8 Hz, 1H), 7.83-7.70 (m, 2H), 7.57-7.47 (m, 2H), 7.45-7.36 (m, 2H), 7.35 7.23 (m, 4H), 7.23-7.08 (m, 3H), 4.92 (s, 2H), 4.58-4.38 (m, 2H), 4.32-4.13 (m, 1H), 3.61 (d, J=5.0, 14.2 Hz, 1H), 3.32-3.23 (m, 1H), 3.22-3.09 (m, 2H), 2.34 (d, J=4.5, 8.9 Hz, 1H), 2.21 (d, J=3.6, 8.7 Hz, 1H), 2.04-1.88 (m, 1H), 1.81-1.53 (m, 4H), 0.87-0.68 (m, 1H), 0.52-0.31 (m, 2H), 0.20-0.00 (m, 2H).

Example 10: Synthesis of N—((S)-1-(((S)-1-(((S)-1-cyano-2-((R)-5,5-dimethyl-2-oxopyrrolidin-3-yl)ethyl)amino)-3-cyclopropyl-1-oxopropan-2-yl)amino)-3-(naphthalen-1-yl)-1-oxopropan-2-yl)pyrazine-2-carboxamide (Compound 118)

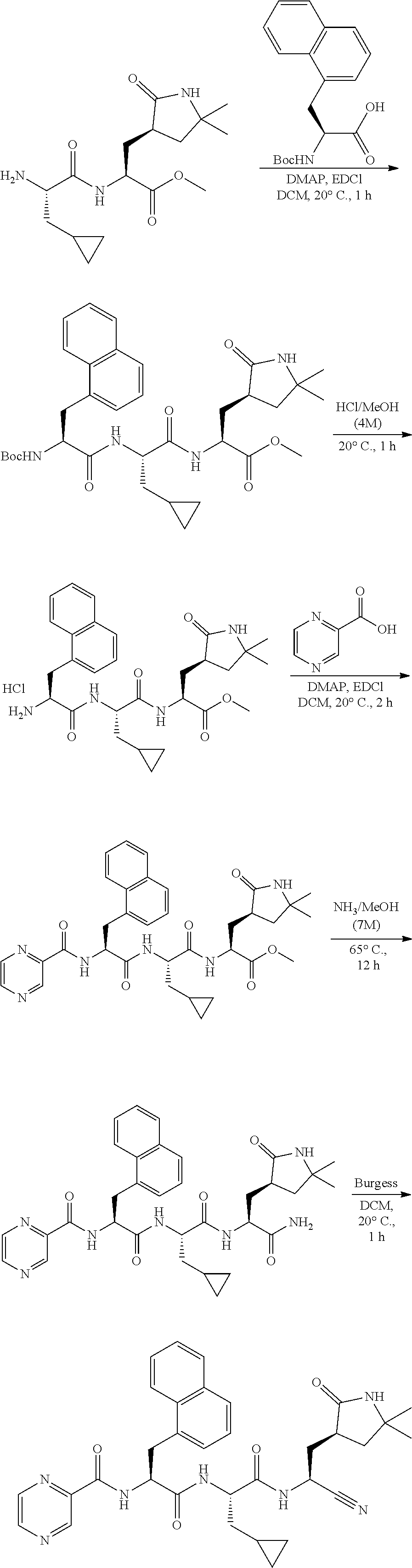

Step 1: (6S,9S,12S)-methyl 9-(cyclopropylmethyl)-12-(((R)-5,5-dimethyl-2-oxopyrrolidin-3-yl)methyl)-2,2-dimethyl-6-(naphthalen-1-ylmethyl)-4,7,10-trioxo-3-oxa-5,8,11-triazatridecan-13-oate

To a solution of methyl (2S)-2-[[(2S)-2-amino-3-cyclopropyl-propanoyl]amino]-3-[(3R)-5,5-dimethyl-2-oxo-pyrrolidin-3-yl]propanoate (550 mg, 1.52 mmol, 1 eq, HCl) and (2S)-2-(tert-butoxycarbonylamino)-3-(1-naphthyl)propanoic acid (527.26 mg, 1.67 mmol, 1.1 eq) in DCM (20 mL) was added DMAP (371.37 mg, 3.04 mmol, 2 eq) and EDCI (582.74 mg, 3.04 mmol, 2 eq). The resulting mixture was stirred at 20° C. for 1 h. Upon completion, the reaction mixture was diluted with H2O (60 mL) and extracted with DCM (40 mL*3). The combined organic layers were washed with brine (50 mL), dried over Na₂SO₄, filtered and concentrated under reduced pressure to give a residue. The residue was purified by column chromatography (SiO₂, Petroleum ether/Ethyl acetate=I/O to 0/1) to give methyl (25)-2 [[(25)-2-[[(2S)-2-(tert-butoxycarbonylamino)-3-(1-naphthyl)propanoyl]amino]-3-cyclopropylpropanoyl]amino]-3-[(3R)-5,5-dimethyl-2-oxo-pyrrolidin-3-yl]propanoate (800 mg, 1.28 mmol, 84.52% yield) as a yellow solid. MS (ESI) m/z 623.5 [M+H]⁺.

Step 2: (S)-methyl 2-((S)-2-((S)-2-amino-3-(naphthalen-1-yl)propanamido)-3-cyclopropylpropanamido)-3-((R)-5,5-dimethyl-2-oxopyrrolidin-3-yl)propanoate

A solution of methyl (2S)-2-[[(2S)-2-[[(2S)-2-(tert-butoxycarbonylamino)-3-(1-naphthyl)propanoyl]amino]-3-cyclopropylpropanoyl]amino]-3-[(3R)-5,5-dimethyl-2-oxo-pyrrolidin-3-yl]propanoate (450 mg, 722.60 umol, 1 eq) in HCl/MeOH (4 M, 20 mL, 110.71 eq) was stirred at 20° C. for 1 h. Upon completion, the reaction mixture was concentrated under reduced pressure to give the product methyl (2S)-2-[[(2S)-2-[[(2S)-2-amino-3-(1-naphthyl)propanoyl]amino]-3-cyclopropyl-propanoyl]amino]-3-[(3R)-5,5-dimethyl-2-oxo-pyrrolidin-3-yl]propanoate (410 mg, crude, HCl) as a yellow solid. MS (ESI) m/z 523.2 [M+H]⁺.

Step 3: (S)-methyl 2-((S)-3-cyclopropyl-2-((S)-3-(naphthalen-1-yl)-2-(pyrazine-2-carboxamido)propanamido)propanamido)-3-((R)-5,5-dimethyl-2-oxopyrrolidin-3-yl)propanoate

To a solution of methyl (2S)-2-[[(2S)-2-[[(2S)-2-amino-3-(1-naphthyl)propanoyl]amino]-3-cyclopropyl-propanoyl]amino]-3-[(3R)-5,5-dimethyl-2-oxo-pyrrolidin-3-yl]propanoate (120 mg, 214.63 umol, 1 eq, HCl) and pyrazine-2-carboxylic acid (29.30 mg, 236.10 umol, 1.1 eq) in DCM (6 mL) was added DMAP (52.44 mg, 429.26 umol, 2 eq) and EDCI (82.29 mg, 429.26 umol, 2 eq). The resulting mixture was stirred at 20° C. for 2 h. Upon completion, the reaction mixture was diluted with H2O (60 mL) and extracted with DCM (40 mL*3). The combined organic layers were washed with brine (50 mL), dried over Na₂SO₄, filtered and concentrated under reduced pressure to give a residue. The residue was purified by prep-TLC (SiO₂, DCM:MeOH=10:1) to give the product methyl (2S)-2-[[(2S)-3-cyclopropyl-2-[[(2S)-3-(1-naphthyl)-2-(pyrazine-2-carbonylamino)propanoyl]amino]propanoyl]amino]-3-[(3R)-5,5-dimethyl-2-oxo-pyrrolidin-3-yl]propanoate (110 mg, 174.96 umol, 81.52% yield) as a white solid. MS (ESI) m/z 629.5 [M+H]⁺.

Step 4: N—((S)-1-(((S)-1-(((S)-1-amino-3-((R)-5,5-dimethyl-2-oxopyrrolidin-3-yl)-1-oxopropan-2-yl)amino)-3-cyclopropyl-1-oxopropan-2-yl)amino)-3-(naphthalen-1-yl)-1-oxopropan-2-yl)pyrazine-2-carboxamide

A solution of methyl (2S)-2-[[(2S)-3-cyclopropyl-2-[[(2S)-3-(1-naphthyl)-2-(pyrazine-2-carbonylamino)propanoyl]amino]propanoyl]amino]-3-[(3R)-5,5-dimethyl-2-oxo-pyrrolidin-3-yl]propanoate (110 mg, 174.96 umol, 1 eq) in NH₃/MeOH (7 M, 15 mL, 600.14 eq) was stirred at 65° C. for 12 h. Upon completion, the reaction mixture was concentrated under reduced pressure to give N-[(1S)-2-[[(1S)-2-[[(1S)-2-amino-1-[[(3R)-5,5-dimethyl-2-oxo-pyrrolidin-3-yl]methyl]-2-oxo-ethyl]amino]-1-(cyclopropylmethyl)-2-oxo-ethyl]amino]-1-(1-naphthylmethyl)-2-oxo-ethyl]pyrazine-2-carboxamide (100 mg, crude) as a yellow solid. MS (ESI) m/z 614.3 [M+H]⁺

Step 5: (N—((S)-1-(((S)-1-(((S)-1-cyano-2-((R)-5,5-dimethyl-2-oxopyrrolidin-3-yl)ethyl)amino)-3-cyclopropyl-1-oxopropan-2-yl)amino)-3-(naphthalen-1-yl)-1-oxopropan-2-yl)pyrazine-2-carboxamide

To a solution of N-[(1S)-2-[[(1S)-2-[[(1S)-2-amino-1-[[(3R)-5,5-dimethyl-2-oxo-pyrrolidin-3-yl]methyl]-2-oxo-ethyl]amino]-1-(cyclopropylmethyl)-2-oxo-ethyl]amino]-1-(1-naphthylmethyl)-2-oxo-ethyl]pyrazine-2-carboxamide (100 mg, 162.94 umol, 1 eq) in DCM (8 mL) was added Burgess reagent (116.49 mg, 488.83 umol, 3 eq). The resulting mixture was stirred at 20° C. for 1 h. Upon completion, the mixture was diluted with H2O (0.5 mL) and then dried with N2 to give a residue. The residue was purified by prep-HPLC (column: Waters Xbridge BEH C18 100*30 mm*10 um; mobile phase: [water(NH₄HCO₃)-ACN]; B %: 30%-60%, 8 min) to give the product N-[(1S)-2-[[(1S)-2-[[(1S)-1-cyano-2-[(3R)-5,5-dimethyl-2-oxo-pyrrolidin-3-yl]ethyl]amino]-1-(cyclopropylmethyl)-2-oxo-ethyl]amino]-1-(1-naphthylmethyl)-2-oxo-ethyl]pyrazine-2-carboxamide (12 mg, 20.06 umol, 12.31% yield, 99.59% purity) as a white solid.

¹H NMR (400 MHz, MeOD-d₄) δ ppm 9.18-9.02 (m, 1H) 8.74 (d, J=2.50 Hz, 1H) 8.58 (dd, J=2.38, 1.55 Hz, 1H) 8.28-8.16 (m, 1H) 7.98-7.98 (m, 1H) 7.78-7.71 (m, 1H) 7.58-7.39 (m, 3H) 7.38-7.30 (m, 1H) 5.10-4.93 (m, 2H) 4.36-4.26 (m, 1H) 3.89-3.78 (m, 1H) 3.58-3.47 (m, 1H) 2.93-2.69 (m, 1H) 2.59-2.21 (m, 1H) 2.17 (dd, J=12.40, 8.58 Hz, 1H) 1.85-1.71 (m, 2H) 1.68-1.48 (m, 2H) 1.33-1.23 (m, 3H) 1.20-1.12 (m, 3H) 0.80-0.67 (m, 1H) 0.59-0.38 (m, 2H) 0.31-0.01 (m, 2H)

Example 11: Synthesis of N—((S)-1-(((S)-1-(((S)-1-cyano-2-((S)-2-oxopyrrolidin-3-yl)ethyl)amino)-3-cyclopropyl-1-oxopropan-2-yl)amino)-3-(naphthalen-1-yl)-1-oxopropan-2-yl)pyrazine-2-carboxamide (Compound 119)

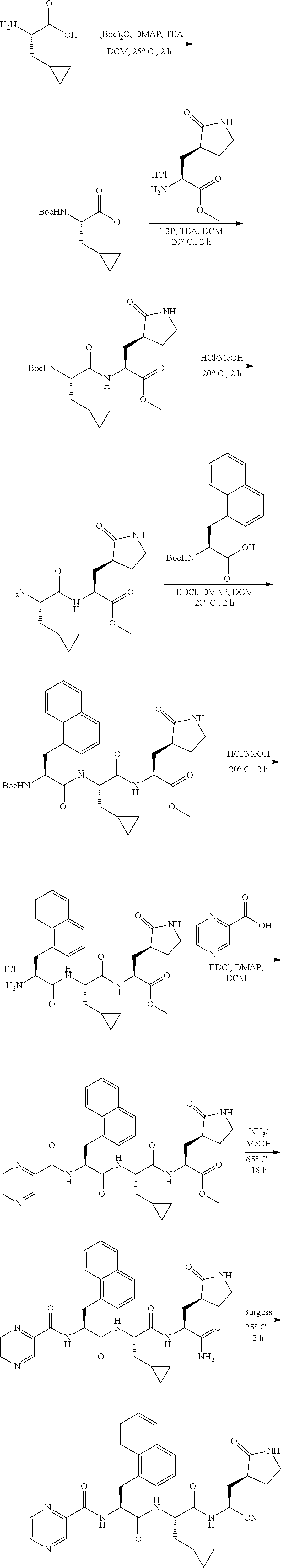

Step 1: (S)-2-((tert-butoxycarbonyl)amino)-3-cyclopropylpropanoic acid

To a solution of (2S)-2-amino-3-cyclopropyl-propanoic acid (12 g, 92.91 mmol, 1 eq), TEA (11.28 g, 111.49 mmol, 15.52 mL, 1.2 eq), and DMAP (2.27 g, 18.58 mmol, 0.2 eq) in DCM (200 mL) was added (Boc)₂₀ (24.33 g, 111.49 mmol, 25.61 mL, 1.2 eq). The mixture was stirred at 20° C. for 1 h. Upon completion, the reaction mixture was poured into H₂O (300 mL) at 20° C., and then the resulting mixture was extracted with DCM (300 mL*3). The combined organic layers were washed with brine (200 mL*2), dried over Na₂SO₄, filtered and concentrated under reduced pressure to give a residue. The residue was purified by column chromatography (SiO₂, Petroleum ether/Ethyl acetate=60/1 to 5/1) to give (2S)-2-(tert-butoxycarbonylamino)-3-cyclopropyl-propanoic acid (20 g, 87.23 mmol, 93.89% yield) as a yellow oil.

Step 2: (S)-methyl 2-((S)-2-((tert-butoxycarbonyl)amino)-3-cyclopropylpropanamido)-3-((S)-2-oxopyrrolidin-3-yl)propanoate

To a solution of (2S)-2-(tert-butoxycarbonylamino)-3-cyclopropyl-propanoic acid (20 g, 87.23 mmol, 1 eq), methyl (2S)-2-amino-3-[(3S)-2-oxopyrrolidin-3-yl]propanoate (19.42 g, 87.23 mmol, 1 eq, HCl), and TEA (26.48 g, 261.70 mmol, 36.42 mL, 3 eq) in DCM (200 mL) was added T3P (138.78 g, 218.08 mmol, 129.70 mL, 50% purity, 2.5 eq) at 0° C. The mixture was stirred at 0° C. for 2 h. Upon completion, the reaction mixture was poured into H₂O (200 mL) at 20° C., and then the resulting mixture was extracted with DCM (200 mL*3). The combined organic layers were washed with brine (200 mL*2), dried over Na₂SO₄, filtered and concentrated under reduced pressure to give a residue. The residue was purified by column chromatography (SiO₂, Petroleum ether/Ethyl acetate=50/1 to 1/1) to give methyl (2S)-2-[[(2S)-2-(tert-butoxycarbonylamino)-3-cyclopropyl-propanoyl]amino]-3-[(3S)-2-oxopyrrolidin-3-yl]propanoate (30 g, 75.48 mmol, 86.53% yield) as a yellow oil.

Step 3: (S)-methyl 2-((S)-2-amino-3-cyclopropylpropanamido)-3-((S)-2-oxopyrrolidin-3-yl)propanoate

A solution of methyl (2S)-2-[[(2S)-2-(tert-butoxycarbonylamino)-3-cyclopropyl-propanoyl]amino]-3-[(3S)-2-oxopyrrolidin-3-yl]propanoate (5 g, 12.58 mmol, 1 eq) in HCl/MeOH (4 M, 50 mL, 15.90 eq) was stirred at 20° C. for 2 h. Upon completion, the reaction mixture was concentrated under reduced pressure to remove solvent to give methyl (2S)-2-[[(2S)-2-amino-3-cyclopropyl-propanoyl]amino]-3-[(3S)-2-oxopyrrolidin-3-yl]propanoate (4.2 g, crude, HCl) as a yellow oil.

Step 4: (6S,9S,12S)-methyl 9-(cyclopropylmethyl)-2,2-dimethyl-6-(naphthalen-1-ylmethyl)-4,7,10-trioxo-12-(((S)-2-oxopyrrolidin-3-yl)methyl)-3-oxa-5,8,11-triazatridecan-13-oate

To a solution of methyl (2S)-2-[[(2S)-2-amino-3-cyclopropyl-propanoyl]amino]-3-[(3S)-2-oxopyrrolidin-3-yl]propanoate (4.1 g, 12.28 mmol, 1 eq, HCl), (2S)-2-(tert-butoxycarbonylamino)-3-(1-naphthyl)propanoic acid (3.49 g, 11.05 mmol, 0.9 eq), DMAP (3.75 g, 30.71 mmol, 2.5 eq) in DCM (40 mL) was added EDCI (4.71 g, 24.56 mmol, 2 eq). The mixture was stirred at 25° C. for 2 h. Upon completion, the reaction mixture was poured into H₂O (80 mL) at 20° C., and then extracted with DCM (100 mL*3). The combined organic layers were washed with brine (80 mL*2), dried over Na₂SO₄, filtered and concentrated under reduced pressure to give a residue. The residue was purified by column chromatography (SiO₂, Petroleum ether/Ethyl acetate=8011 to 1/1) to give methyl (2S)-2-[[(2S)-2-[[(2S)-2-(tert-butoxycarbonylamino)-3-(1-naphthyl)propanoyl]amino]-3-cyclopropyl-propanoyl]amino]-3-[(3S)-2-oxopyrrolidin-3-yl]propanoate (4.4 g, 7.40 mmol, 60.24% yield) as a white solid. MS (ESI) m/z 595.3 [M+H]⁺.

Step 5: (S)-methyl 2-((S)-2-((S)-2-amino-3-(naphthalen-1-yl)propanamido)-3-cyclopropylpropanamido)-3-((S)-2-oxopyrrolidin-3-yl)propanoate

A solution of methyl (2S)-2-[[(2S)-2-[[(2S)-2-(tert-butoxycarbonylamino)-3-(1-naphthyl)propanoyl]amino]-3-cyclopropyl-propanoyl]amino]-3-[(3S)-2-oxopyrrolidin-3-yl]propanoate (3 g, 5.04 mmol, 1 eq) in HCl/MeOH (4 M, 30 mL, 23.79 eq) was stirred at 25° C. for 2 h. Upon completion, the reaction mixture was concentrated under reduced pressure to remove solvent to give methyl (2S)-2-[[(2S)-2-[[(2S)-2-amino-3-(1-naphthyl)propanoyl]amino]-3-cyclopropyl-propanoyl]amino]-3-[(3S)-2-oxopyrrolidin-3-yl]propanoate (2.1 g, crude, HCl) as a yellow solid.

Step 6: (S)-methyl 2-((S)-3-cyclopropyl-2-((S)-3-(naphthalen-1-yl)-2-(pyrazine-2-carboxamido)propanamido)propanamido)-3-((S)-2-oxopyrrolidin-3-yl)propanoate

To a solution of methyl (2S)-2-[[(2S)-2-[[(2S)-2-amino-3-(1-naphthyl)propanoyl]amino]-3-cyclopropyl-propanoyl]amino]-3-[(3S)-2-oxopyrrolidin-3-yl]propanoate (250 mg, 470.77 umol, 1 eq, HCl), pyrazine-2-carboxylic acid (52.58 mg, 423.70 umol, 0.9 eq), DMAP (143.78 mg, 1.18 mmol, 2.5 eq) in DCM (5 mL) was added EDCI (180.50 mg, 941.54 umol, 2 eq). The mixture was stirred at 25° C. for 2 h. Upon completion, the reaction mixture was poured into H₂O (25 mL) at 20° C., and then extracted with DCM (30 mL*3). The combined organic layers were washed with brine (25 mL*2), dried over Na₂SO₄, filtered and concentrated under reduced pressure to give a residue. The residue was purified by column chromatography (SiO₂, DCM/MeOH=I/O to 80/1) to give methyl (2S)-2-[[(2S)-3-cyclopropyl-2-[[(2S)-3-(1-naphthyl)-2-(pyrazine-2-carbonylamino)propanoyl]amino]propanoyl]amino]-3-[(3S)-2-oxopyrrolidin-3-yl]propanoate (240 mg, 399.56 umol, 84.87% yield) as a yellow solid. MS (ESI) m/z 601.3 [M+H]⁺.

Step 7: N—((S)-1-(((S)-1-(((S)-1-amino-1-oxo-3-((S)-2-oxopyrrolidin-3-yl)propan-2-yl)amino)-3-cyclopropyl-1-oxopropan-2-yl)amino)-3-(naphthalen-1-yl)-1-oxopropan-2-yl)pyrazine-2-carboxamide

A solution of methyl (2S)-2-[[(2S)-3-cyclopropyl-2-[[(2S)-3-(1-naphthyl)-2-(pyrazine-2-carbonylamino)propanoyl]amino]propanoyl]amino]-3-[(3S)-2-oxopyrrolidin-3-yl]propanoate (230 mg, 382.91 umol, 1 eq) in NH₃/MeOH (7 M, 4.60 mL, 84.09 eq) was stirred at 65° C. for 18 h. Upon completion, the reaction mixture was concentrated under reduced pressure to remove solvent to give N-[(1S)-2-[[(1S)-2-[[(1S)-2-amino-2-oxo-1-[[(3S)-2-oxopyrrolidin-3-yl]methyl]ethyl]amino]-1-(cyclopropylmethyl)-2-oxo-ethyl]amino]-1-(1-naphthylmethyl)-2-oxo-ethyl]pyrazine-2-carboxamide (220 mg, crude) as a white solid. MS (ESI) m/z 586.3 [M+H]⁺.

Step 8: N—((S)-1-(((S)-1-(((S)-1-cyano-2-((S)-2-oxopyrrolidin-3-yl)ethyl)amino)-3-cyclopropyl-1-oxopropan-2-yl)amino)-3-(naphthalen-1-yl)-1-oxopropan-2-yl)pyrazine-2-carboxamide

To a solution of N-[(1S)-2-[[(1S)-2-[[(1S)-2-amino-2-oxo-1-[[(3S)-2-oxopyrrolidin-3-yl]methyl]ethyl]amino]-1-(cyclopropylmethyl)-2-oxo-ethyl]amino]-1-(1-naphthylmethyl)-2-oxo-ethyl]pyrazine-2-carboxamide (210 mg, 358.57 umol, 1 eq) in DCM (6 mL) was added Burgess reagent (170.90 mg, 717.15 umol, 2 eq). The mixture was stirred at 25° C. for 2 h. Upon completion, the reaction mixture was concentrated under N2 to remove solvent. The residue was purified by prep-HPLC (column: Waters Xbridge BEH C18 250*50 mm*10 um; mobile phase: [water(NH₄HCO₃)-ACN]; B %: 30%-55%, 10 min) to give N-[(1S)-2-[[(1S)-2-[[(1S)-1-cyano-2-[(3S)-2-oxopyrrolidin-3-yl]ethyl]amino]-1-(cyclopropylmethyl)-2-oxo-ethyl]amino]-1-(1-naphthylmethyl)-2-oxo-ethyl]pyrazine-2-carboxamide (70 mg, 122.70 umol, 34.22% yield, 99.5% purity) as a white solid. MS (ESI) m/z 568.1 [M+H]⁺.

¹H NMR (400 MHz, DMSO-d₆) δ=9.05 (d, J=1.3 Hz, 1H), 8.90 (d, J=7.8 Hz, 1H), 8.88-8.81 (m, 2H), 8.70 (d, J=1.5, 2.3 Hz, 1H), 8.54 (d, J=7.7 Hz, 1H), 8.21 (d, J=7.9 Hz, 1H), 7.91-7.84 (m, 1H), 7.80-7.67 (m, 2H), 7.56-7.43 (m, 2H), 7.42-7.26 (m, 2H), 5.13-4.87 (m, 2H), 4.34 (q, J=7.3 Hz, 1H), 3.66 (d, J=4.8, 14.2 Hz, 1H), 3.51-3.45 (m, 1H), 3.21-3.04 (m, 2H), 2.40-2.31 (m, 1H), 2.24-2.03 (m, 2H), 1.90-1.58 (m, 3H), 1.44 (d, J=7.0, 14.0 Hz, 1H), 0.83-0.64 (m, 1H), 0.52-0.29 (m, 2H), 0.22-0.03 (m, 2H).

Example 12: Synthesis of N—((S)-1-(((S)-1-(((S)-1-cyano-2-((R)-5,5-dimethyl-2-oxopyrrolidin-3-yl)ethyl)amino)-3-cyclopropyl-1-oxopropan-2-yl)amino)-3-(naphthalen-1-yl)-1-oxopropan-2-yl)-5-methylisoxazole-3-carboxamide (Compound 120)

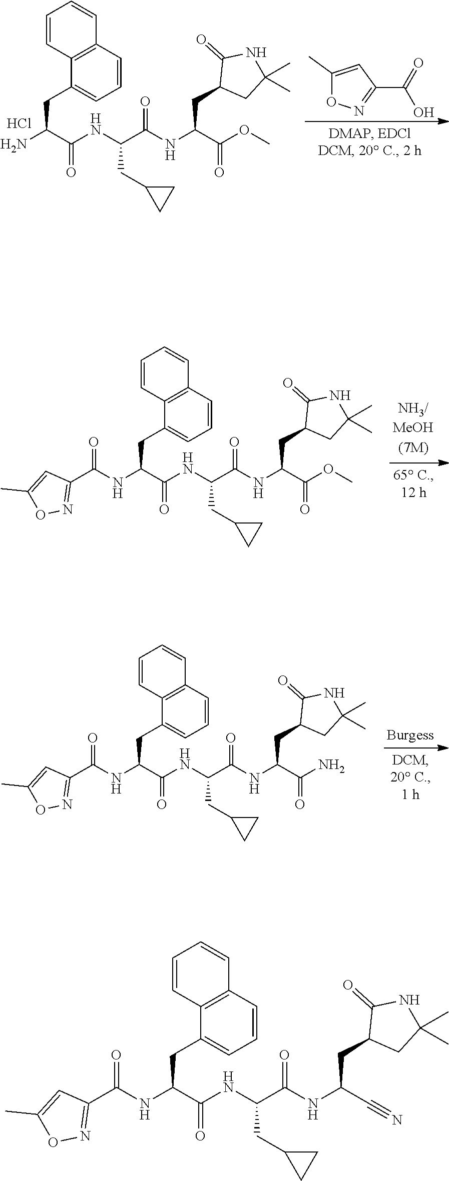

Step 1: (S)-methyl 2-((S)-3-cyclopropyl-2-((S)-2-(5-methylisoxazole-3-carboxamido)-3-(naphthalen-1-yl)propanamido)propanamido)-3-((R)-5,5-dimethyl-2-oxopyrrolidin-3-yl)propanoate

To a solution of methyl (2S)-2-[[(2S)-2-[[(2S)-2-amino-3-(1-naphthyl)propanoyl]amino]-3-cyclopropyl-propanoyl]amino]-3-[(3R)-5,5-dimethyl-2-oxo-pyrrolidin-3-yl]propanoate (100 mg, 178.86 umol, 1 eq, HCl) and 5-methylisoxazole-3-carboxylic acid (25.01 mg, 196.75 umol, 1.1 eq) in DCM (6 mL) was added DMAP (43.70 mg, 357.72 umol, 2 eq) and EDCI (68.58 mg, 357.72 umol, 2 eq). The resulting mixture was stirred at 20° C. for 2 h. Upon completion, the reaction mixture was diluted with H₂O (60 mL) and extracted with DCM (40 mL*3). The combined organic layers were washed with brine (50 mL), dried over Na₂SO₄, filtered and concentrated under reduced pressure to give a residue. The residue was purified by prep-TLC (SiO₂, DCM:MeOH=10:1) to give the product methyl (2S)-2-[[(2S)-3-cyclopropyl-2-[[(2S)-2-[(5-methylisoxazole-3-carbonyl)amino]-3-(1-naphthyl)propanoyl]amino]propanoyl]amino]-3-[(3R)-5,5-dimethyl-2-oxo-pyrrolidin-3-yl]propanoate (90 mg, 142.47 umol, 79.65% yield) as a white solid. MS (ESI) m/z 632.3 [M+H]⁺.

Step 2: N—((S)-1-(((S)-1-(((S)-1-amino-3-((R)-5,5-dimethyl-2-oxopyrrolidin-3-yl)-1-oxopropan-2-yl)amino)-3-cyclopropyl-1-oxopropan-2-yl)amino)-3-(naphthalen-1-yl)-1-oxopropan-2-yl)-5-methylisoxazole-3-carboxamide

A solution of methyl (2S)-2-[[(2S)-3-cyclopropyl-2-[[(2S)-2-[(5-methylisoxazole-3-carbonyl)amino]-3-(1-naphthyl)propanoyl]amino]propanoyl]amino]-3-[(3R)-5,5-dimethyl-2-oxo-pyrrolidin-3-yl]propanoate (80 mg, 126.64 umol, 1 eq) in NH₃/MeOH (7 M, 16.00 mL, 884.40 eq) was stirred at 65° C. for 12 h. Upon completion, the reaction mixture was concentrated under reduced pressure to give the product N-[(1S)-2-[[(1S)-2-[[(1S)-2-amino-1-[[(3R)-5,5-dimethyl-2-oxo-pyrrolidin-3-yl]methyl]-2-oxo-ethyl]amino]-1-(cyclopropylmethyl)-2-oxo-ethyl]amino]-1-(1-naphthylmethyl)-2-oxo-ethyl]-5-methyl-isoxazole-3-carboxamide (80 mg, crude) as a yellow solid. MS (ESI) m/z 617.3 [M+H]⁺.

Step 3: N—((S)-1-(((S)-1-(((S)-1-cyano-2-((R)-5,5-dimethyl-2-oxopyrrolidin-3-yl)ethyl)amino)-3-cyclopropyl-1-oxopropan-2-yl)amino)-3-(naphthalen-1-yl)-1-oxopropan-2-yl)-5-methylisoxazole-3-carboxamide

To a solution of N-[(1S)-2-[[(1S)-2-[[(1S)-2-amino-1-[[(3R)-5,5-dimethyl-2-oxo-pyrrolidin-3-yl]methyl]-2-oxo-ethyl]amino]-1-(cyclopropylmethyl)-2-oxo-ethyl]amino]-1-(1-naphthylmethyl)-2-oxo-ethyl]-5-methyl-isoxazole-3-carboxamide (80 mg, 129.72 umol, 1 eq) in DCM (6 mL) was added Burgess reagent (123.65 mg, 518.89 umol, 4 eq), and the mixture was stirred at 20° C. for 1 h. Upon completion, the mixture was diluted with H₂O (0.5 mL) and then dried with N₂ to give a residue. The residue was purified by prep-HPLC (column: Waters Xbridge BEH C18 100*30 mm*10 um; mobile phase: [water(NH₄HCO₃)-ACN]; B %: 30%-60%, 8 min) to give the product N-[(1S)-2-[[(1S)-2-[[(1S)-1-cyano-2-[(3R)-5,5-dimethyl-2-oxo-pyrrolidin-3-yl]ethyl]amino]-1-(cyclopropylmethyl)-2-oxo-ethyl]amino]-1-(1-naphthylmethyl)-2-oxo-ethyl]-5-methyl-isoxazole-3-carboxamide (8 mg, 13.36 umol, 10.30% yield, 100% purity) as a white solid. MS (ESI) m/z 599.1 [M+H]⁺.

¹H NMR (400 MHz, MeOD-d₄ 8 ppm 8.21 (d, J=8.34 Hz, 1H) 7.90-7.82 (m, 1H) 7.81-7.61 (m, 1H) 7.60-7.44 (m, 2H) 7.42-7.33 (m, 2H) 6.52-6.30 (m, 1H) 5.08-4.90 (m, 2H) 4.42-4.14 (m, 1H) 3.84-3.60 (m, 1H) 3.48-3.37 (m, 1H) 2.88-2.78 (m, 1H) 2.50-2.41 (m, 3H) 2.42-2.29 (m, 1H) 2.25-2.05 (m, 1H) 1.96-1.72 (m, 2H) 1.69 1.44 (m, 2H) 1.30-1.24 (m, 3H) 1.15 (s, 3H) 0.80-0.64 (m, 1H) 0.56-0.39 (m, 2H) 0.29-0.05 (m, 2H)

Example 13: Synthesis of N—((S)-1-(((S)-1-(((S)-1-cyano-2-((S)-2-oxopyrrolidin-3-yl)ethyl)amino)-3-cyclopropyl-1-oxopropan-2-yl)amino)-3-(naphthalen-1-yl)-1-oxopropan-2-yl)-5-methylisoxazole-3-carboxamide (Compound 121)

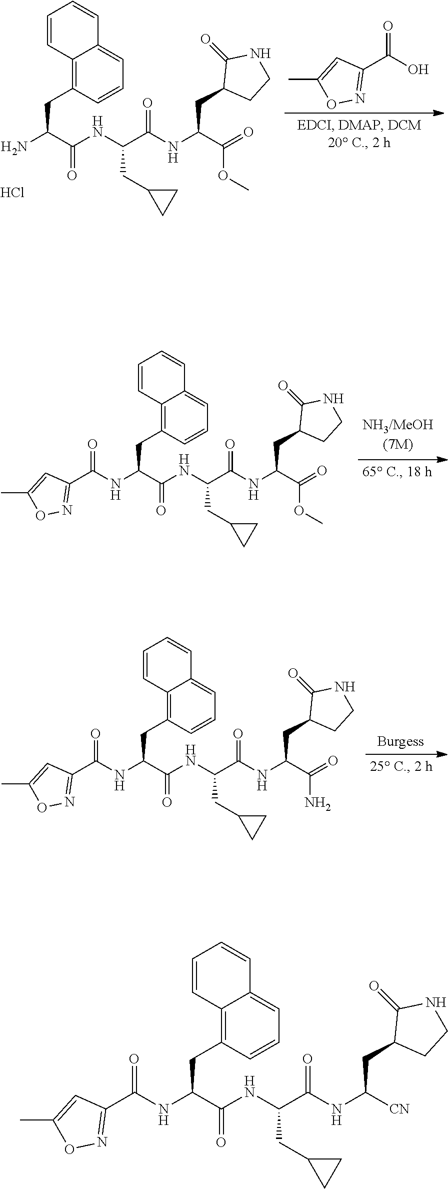

Step 1: (S)-methyl 2-((S)-3-cyclopropyl-2-((S)-2-(5-methylisoxazole-3-carboxamido)-3-(naphthalen-1-yl)propanamido)propanamido)-3-((S)-2-oxopyrrolidin-3-yl)propanoate

To a solution of methyl (2S)-2-[[(2S)-2-[[(2S)-2-amino-3-(1-naphthyl)propanoyl]amino]-3-cyclopropyl-propanoyl]amino]-3-[(3S)-2-oxopyrrolidin-3-yl]propanoate (250 mg, 470.77 umol, 1 eq, HCl), 5-methylisoxazole-3-carboxylic acid (53.85 mg, 423.70 umol, 0.9 eq), DMAP (143.78 mg, 1.18 mmol, 2.5 eq) in DCM (5 mL) was added EDCI (180.50 mg, 941.54 umol, 2 eq). The mixture was stirred at 25° C. for 2 h. Upon completion, the reaction mixture was poured into H₂O (25 mL) at 25° C., and then extracted with DCM (30 mL*3). The combined organic layers were washed with brine (20 mL*2), dried over Na₂SO₄, filtered and concentrated under reduced pressure to give a residue. The residue was purified by column chromatography (SiO₂, DCM/MeOH=100/0 to 4/1) to give methyl (2S)-2-[[(2S)-3-cyclopropyl-2-[[(2S)-2-[(5-methylisoxazole-3-carbonyl)amino]-3-(1-naphthyl)propanoyl]amino]propanoyl]amino]-3-[(3S)-2-oxopyrrolidin-3-yl]propanoate (250 mg, 414.14 umol, 87.97% yield) as a white solid. MS (ESI) m/z 604.3 [M+H]⁺.

Step 2: N—((S)-1-(((S)-1-(((S)-1-amino-1-oxo-3-((S)-2-oxopyrrolidin-3-yl)propan-2-yl)amino)-3-cyclopropyl-1-oxopropan-2-yl)amino)-3-(naphthalen-1-yl)-1-oxopropan-2-yl)-5-methylisoxazole-3-carboxamide

A solution of methyl (2S)-2-[[(2S)-3-cyclopropyl-2-[[(2S)-2-[(5-methylisoxazole-3-carbonyl)amino]-3-(1-naphthyl)propanoyl]amino]propanoyl]amino]-3-[(3S)-2-oxopyrrolidin-3-yl]propanoate (240 mg, 397.57 umol, 1 eq) in NH₃/MeOH (7 M, 6 mL, 105.64 eq) was stirred at 65° C. for 18 h. Upon completion, the reaction mixture was concentrated under reduced pressure to remove solvent to give N-[(1S)-2-[[(1S)-2-[[(1S)-2-amino-2-oxo-1-[[(3S)-2-oxopyrrolidin-3-yl]methyl]ethyl]amino]-1-(cyclopropylmethyl)-2-oxo-ethyl]amino]-1-(1-naphthylmethyl)-2-oxo-ethyl]-5-methyl-isoxazole-3-carboxamide (230 mg, crude) was obtained as a white solid. MS (ESI) m/z 589.3 [M+H]⁺.

Step 3: N—((S)-1-(((S)-1-(((S)-1-cyano-2-((S)-2-oxopyrrolidin-3-yl)ethyl)amino)-3-cyclopropyl-1-oxopropan-2-yl)amino)-3-(naphthalen-1-yl)-1-oxopropan-2-yl)-5-methylisoxazole-3-carboxamide

To a solution of N-[(1S)-2-[[(1S)-2-[[(1S)-2-amino-2-oxo-1-[[(3S)-2-oxopyrrolidin-3-yl]methyl]ethyl]amino]-1-(cyclopropylmethyl)-2-oxo-ethyl]amino]-1-(1-naphthylmethyl)-2-oxo-ethyl]-5-methyl-isoxazole-3-carboxamide (230 mg, 390.72 umol, 1 eq) in DCM (6 mL) was added Burgess reagent (186.22 mg, 781.45 umol, 2 eq). The mixture was stirred at 25° C. for 2 h. Upon completion, the reaction mixture was concentrated under N2 to remove solvent. The residue was purified by prep-HPLC (column: Waters Xbridge BEH C18 250*50 mm*10 um; mobile phase: [water (NH₄HCO₃)-ACN]; B %: 35%-55%, 10 min) to give N-[(1S)-2-[[(1S)-2-[[(1S)-1-cyano-2-[(3S)-2-oxopyrrolidin-3-yl]ethyl]amino]-1-(cyclopropylmethyl)-2-oxo-ethyl]amino]-1-(1-naphthylmethyl)-2-oxo-ethyl]-5-methyl-isoxazole-3-carboxamide (130 mg, 225.77 umol, 57.78% yield, 99.1% purity) as a white solid. MS (ESI) m/z 571.1 [M+H]⁺.

¹H NMR (400 MHz, DMSO-d₆) δ=8.89 (d, J=7.8 Hz, 1H), 8.73 (d, J=8.6 Hz, 1H), 8.40 (d, J=7.5 Hz, 1H), 8.20 (d, J=8.3 Hz, 1H), 7.90 (d, J=7.9 Hz, 1H), 7.80-7.66 (m, 2H), 7.61-7.47 (m, 2H), 7.44-7.31 (m, 2H), 6.43 (s, 1H), 5.05-4.81 (m, 2H), 4.34 (q, J =7.1 Hz, 1H), 3.63 (d, J=3.9, 14.2 Hz, 1H), 3.42-3.38 (m, 1H), 3.13 (q, J=9.3 Hz, 2H), 2.42 (s, 4H), 2.22-2.05 (m, 2H), 1.87-1.61 (m, 3H), 1.45 (d, J=6.9, 13.9 Hz, 1H), 0.73 (d, J=6.0 Hz, 1H), 0.41 (d, J=6.2 Hz, 2H), 0.23--0.03 (m, 2H).

¹H NMR (400 MHz, DMSO-d₆XDMSO+H₂O) δ=8.96-8.88 (m, 1H), 8.75 8.68 (m, 1H), 8.44-8.36 (m, 1H), 8.17 (d, J=8.3 Hz, 1H), 7.89 (d, J=8.0 Hz, 1H), 7.76 (d, J=7.9 Hz, 1H), 7.71-7.67 (m, 1H), 7.60-7.46 (m, 2H), 7.44-7.30 (m, 2H), 6.40 (s, 1H), 5.02-4.77 (m, 2H), 4.30 (t, J=7.0 Hz, 1H), 3.66-3.64 (m, 1H), 3.37 (d, J=10.0, 14.2 Hz, 1H), 3.21-3.04 (m, 2H), 2.42-2.29 (m, 4H), 2.19-2.04 (m, 2H), 1.86-1.60 (m, 3H), 1.50 1.36 (m, 1H), 0.79-0.63 (m, 1H), 0.47-0.32 (m, 2H), 0.17-0.00 (m, 2H).

Example 14: Synthesis of N-[(1S)-2-[[(1S)-2-[[(1S)-1-cyano-2-[(3S)-2-oxo-3-piperidyl]ethyl]amino]-1-(cyclopropylmethyl)-2-oxo-ethyl amino]-1-[(4-fluorophenyl)methyl]-2-oxo-ethyl]pyrazine-2-carboxamide (Compound 122)

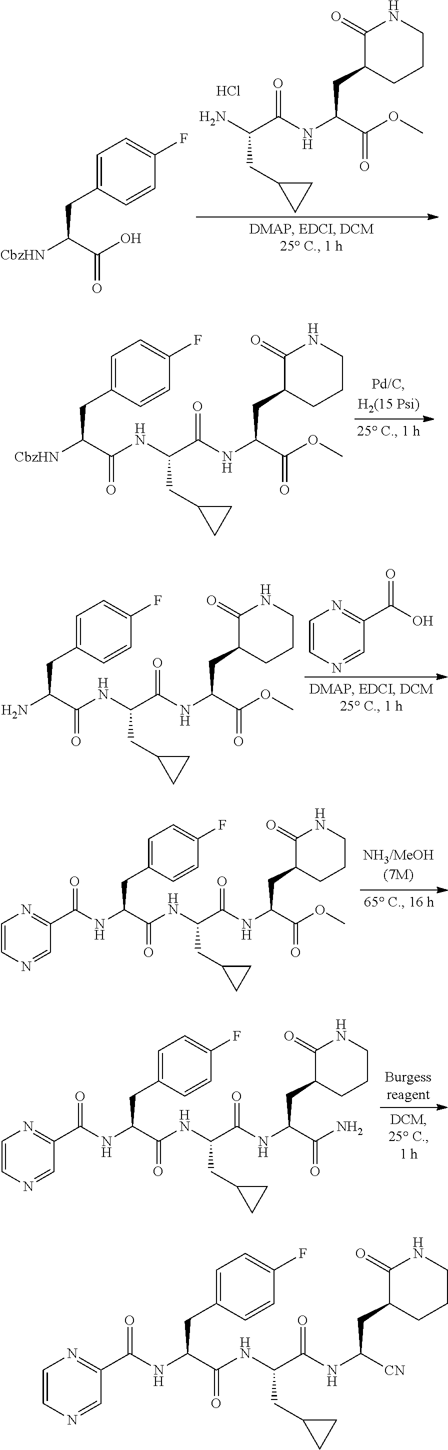

Step 1: methyl (2S)-2-[[(2S)-2-[[(2S)-2-(benzyloxycarbonylamino)-3-(4-fluorophenyl)propanoyl]amino]-3-cyclopropyl-propanoyl]amino]-3-[(3S)-2-oxo-3-piperidyl]propanoate

To a solution of (2S)-2-(benzyloxycarbonylamino)-3-(4-fluorophenyl)propanoic acid (1 g, 3.15 mmol, 1 eq) and methyl (2S)-2-[[(2S)-2-amino-3-cyclopropyl-propanoyl]amino]-3-[(3S)-2-oxo-3-piperidyl]propanoate (1.18 g, 3.78 mmol, 1.2 eq) in DCM (20 mL) was added DMAP (1.16 g, 9.45 mmol, 3 eq) and EDCI (1.21 g, 6.30 mmol, 2 eq). The mixture was stirred at 25° C. for 1 h. Upon completion, the reaction mixture was quenched by addition H₂O (50 mL) and extracted with DCM (15 mL*8). The combined organic layers were dried over Na₂SO₄, filtered and concentrated under reduced pressure and was purified by column chromatography (SiO₂, Petroleum ether/Ethyl acetate=9/1 to 5/1 to 4/1 to 0/1) to give methyl (2S)-2-[[(2S)-2-[[(2S)-2-(benzyloxycarbonylamino)-3-(4-fluorophenyl)propanoyl]amino]-3-cyclopropyl-propanoyl]amino]-3-[(3S)-2-oxo-3-piperidyl]propanoate (1.2 g, 1.81 mmol, 57.36% yield, 92% purity) as yellow solid. MS (ESI) m/z 611.2 [M+H]⁺

Step 2: methyl (2S)-2-[[(2S)-2-[[(2S)-2-amino-3-(4-fluorophenyl)propanoyl]amino]-3-cyclopropyl-propanoyl]amino]-3-[(3S)-2-oxo-3-piperidyl]propanoate

To a solution of methyl (2S)-2-[[(2S)-2-[[(2S)-2-(benzyloxycarbonylamino)-3-(4-fluorophenyl)propanoyl]amino]-3-cyclopropyl-propanoyl]amino]-3-[(3S)-2-oxo-3-piperidyl]propanoate (1.16 g, 1.90 mmol, 1 eq) in IPA (15 mL) was added Pd/C (2.24 g, 1.90 mmol, 10% purity, 1.00 eq) under N₂ atmosphere. The suspension was degassed and purged with H₂ (3.84 mg, 1.90 mmol, 1 eq) for 3 times. The mixture was stirred under H₂ (15 Psi) at 25° C. for 1 h. Upon completion, the combined organic layers were concentrated under reduced pressure to give the crude product methyl (2S)-2-[[(2S)-2-[[(2S)-2-amino-3-(4-fluorophenyl)propanoyl]amino]-3-cyclopropyl-propanoyl]amino]-3-[(3S)-2-oxo-3-piperidyl]propanoate (810 mg, crude) as white solid MS (ESI) m/z 477.2 [M+H]⁺

Step 3: methyl (2S)-2-[[(2S)-3-cyclopropyl-2-[[(2S)-3-(4-fluorophenyl)-2-(pyrazine-2-carbonylamino)propanoyl]amino]propanoyl]amino]-3-[(3S)-2-oxo-3-piperidyl]propanoate

To a solution of methyl (2S)-2-[[(2S)-2-[[(2S)-2-amino-3-(4-fluorophenyl)propanoyl]amino]-3-cyclopropyl-propanoyl]amino]-3-[(3S)-2-oxo-3-piperidyl]propanoate (810 mg, 1.70 mmol, 1 eq) and pyrazine-2-carboxylic acid (316.40 mg, 2.55 mmol, 1.5 eq) in DCM (15 mL) was added DMAP (622.98 mg, 5.10 mmol, 3 eq) and EDCI (651.69 mg, 3.40 mmol, 2 eq). The mixture was stirred at 25° C. for 1 h. Upon completion, the reaction mixture was quenched by addition H₂O (40 mL) and extracted with DCM (10 mL*6). The combined organic layers were dried over Na₂SO₄, filtered and concentrated under reduced pressure and was purified by column chromatography (SiO₂, Petroleum ether/Ethyl acetate=9/1 to 4/1 to 1/1 to 0/1 and then Dichloromethane/Methanol=10/1) to give the product methyl (2S)-2-[[(2S)-3-cyclopropyl-2-[[(2S)-3-(4-fluorophenyl)-2-(pyrazine-2-carbonylamino)propanoyl]amino]propanoyl]amino]-3-[(3S)-2-oxo-3-piperidyl]propanoate (940 mg, 1.61 mmol, 94.92% yield) as white solid. MS (ESI) m/z 583.3 [M+H]⁺

Step 4: N-[(1S)-2-[[(1S)-2-[[(1S)-2-amino-2-oxo-1-[[(3S)-2-oxo-3-piperidyl]methyl]ethyl]amino]-1-(cyclopropylmethyl)-2-oxo-ethyl]amino]-1-[(4-fluorophenyl)methyl]-2-oxo-ethyl]pyrazine-2-carboxamide

The methyl (2S)-2-[[(2S)-3-cyclopropyl-2-[[(2S)-3-(4-fluorophenyl)-2-(pyrazine-2-carbonylamino)propanoyl]amino]propanoyl]amino]-3-[(3S)-2-oxo-3-piperidyl]propanoate (250 mg, 429.09 umol, 1 eq) in NH₃/MeOH (7 M, 12 mL, 195.76 eq) was stirred at 65° C. for 16 h. Upon completion, the reaction mixture was concentrated under pressure reduced to get the crude product N-[(1S)-2-[[(1S)-2-[[(1S)-2-amino-2-oxo-1-[[(3S)-2-oxo-3-piperidyl]methyl]ethyl]amino]-1-(cyclopropylmethyl)-2-oxo-ethyl]amino]-1-[(4-fluorophenyl)methyl]-2-oxo-ethyl]pyrazine-2-carboxamide (227 mg, crude) as white solid. MS (ESI) m/z 568.3 [M+H]⁺

Step 5: N-[(1S)-2-[[(1S)-2-[[(1S)-1-cyano-2-[(3S)-2-oxo-3-piperidyl]ethyl]amino]-1-(cyclopropylmethyl)-2-oxo-ethyl]amino]-1-[(4-fluorophenyl)methyl]-2-oxo-ethyl]pyrazine-2-carboxamide

The N-[(1S)-2-[[(1S)-2-[[(1S)-2-amino-2-oxo-1-[[(3S)-2-oxo-3-piperidyl]methyl]ethyl]amino]-1-(cyclopropylmethyl)-2-oxo-ethyl]amino]-1-[(4-fluorophenyl)methyl]-2-oxo-ethyl]pyrazine-2-carboxamide (227 mg, 399.92 umol, 1 eq) in DCM (5 mL) was added Burgess reagent (285.92 mg, 1.20 mmol, 3 eq), the solution was stirred at 25° C. for 1 h. Upon completion, the reaction mixture was quenched by addition H₂O (0.5 mL) and blow-dried with N2 and was purified by prep-HPLC (column: Phenomenex C18 75*30 mm*3 um; mobile phase: [water (NH₄HCO₃)-ACN]; B %: 18%-38%, 10 min) to get N-[(1S)-2-[[(1S)-2-[[(1S)-1-cyano-2-[(3S)-2-oxo-3-piperidyl]ethyl]amino]-1-(cyclopropylmethyl)-2-oxo-ethyl]amino]-1-[(4-fluorophenyl)methyl]-2-oxo-ethyl]pyrazine-2-carboxamide (85 mg, 154.66 umol, 38.67% yield, 100% purity) as white solid. MS (ESI) m/z 550.1 [M+H]⁺

¹H NMR (400 MHz, DMSO-d₆) δ=9.12 (d, J=1.3 Hz, 1H), 8.98-8.83 (m, 2H), 8.73 (dd, J=1.5, 2.3 Hz, 1H), 8.67 (d, J=8.4 Hz, 1H), 8.54 (br d, J=7.6 Hz, 1H), 7.55 (br s, 1H), 7.23 (dd, J=5.7, 8.4 Hz, 2H), 7.01 (t, J=8.9 Hz, 2H), 5.21-4.94 (m, 1H), 4.83 (dt, J=4.6, 8.3 Hz, 1H), 4.30 (q, J=7.3 Hz, 1H), 3.17-2.99 (m, 4H), 2.29-2.16 (m, 2H), 1.91 1.52 (m, 5H), 1.49-1.25 (m, 2H), 0.82-0.59 (m, 1H), 0.40 (q, J=7.0 Hz, 2H), 0.21--0.06 (m, 2H).

Example 15: Synthesis of N—((S)-1-(((S)-1-(((S)-1-cyano-2-((R)-5,5-dimethyl-2-oxopyrrolidin-3-yl)ethyl)amino)-3-cyclopropyl-1-oxopropan-2-yl)amino)-3-(4-tluorophenyl)-1-oxopropan-2-yl)pyrazine-2-carboxamide (Compound 124)

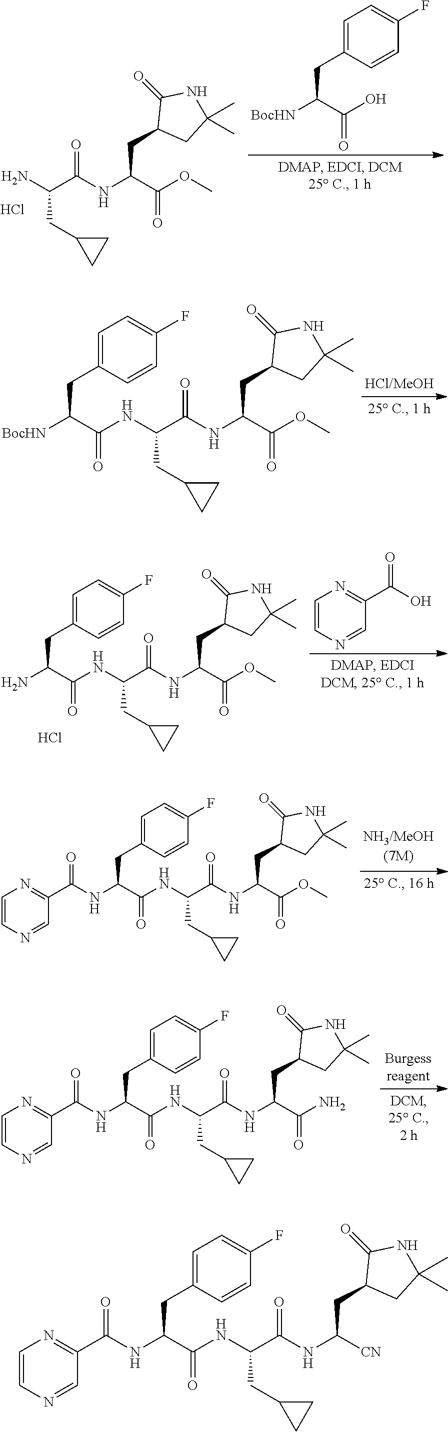

Step 1: (6S,9S,12S)-methyl 9-(cyclopropylmethyl)-12-(((R)-5,5-dimethyl-2-oxopyrrolidin-3-yl)methyl)-6-(4-fluorobenzyl)-2,2-dimethyl-4,7,10-trioxo-3-oxa-5,8,11-triazatridecan-13-oate

To a solution of methyl (2S)-2-[[(2S)-2-amino-3-cyclopropyl-propanoyl]amino]-3-[(3R)-5,5-dimethyl-2-oxo-pyrrolidin-3-yl] propanoate (300 mg, 829.04 umol, 1 eq, HCl) and (2S)-2-(tert-butoxycarbonylamino)-3-(4-fluorophenyl) propanoic acid (281.84 mg, 994.85 umol, 1.2 eq) in DCM (8 mL) was added DMAP (303.85 mg, 2.49 mmol, 3 eq) and EDCI (317.86 mg, 1.66 mmol, 2 eq). The resulting mixture was stirred at 25° C. for 1 h. Upon the reaction complement, the mixture was adjusted pH-1 with aq.HCl (5 mL, 1M), and water (15 mL) was added and extracted with DCM (7 mL*3). The organic phase was adjusted pH-7 with sat. NaHCO₃(7 mL), and then the organic phase was concentration in vacuum and was purified by column (SiO₂, PE:EA=1:0 to 0:1) to obtained (6S,9S,12S)-methyl 9-(cyclopropylmethyl)-12-(((R)-5,5-dimethyl-2-oxopyrrolidin-3-yl) methyl)-6-(4-fluorobenzyl)-2,2-dimethyl-4,7,10-trioxo-3-oxa-5,8,11-triazatridecan-13-oate (400 mg, 575.61 umol, 69.43% yield, 85% purity) as a white solid. MS (ESI) m/z 591.3 [M+H]⁺

Step 2: (S)-methyl 2-((S)-2-((S)-2-amino-3-(4-fluorophenyl)propanamido)-3-cyclopropylpropanamido)-3-((R)-5,5-dimethyl-2-oxopyrrolidin-3-yl)propanoate

A solution of methyl (2S)-2-[[(2S)-2-[[(2S)-2-(tert-butoxycarbonylamino)-3-(4-fluorophenyl) propanoyl]amino]-3-cyclopropyl-propanoyl]amino]-3-[(3R)-5,5-dimethyl-2-oxo-pyrrolidin-3-yl]propanoate (200 mg, 338.59 umol, 1 eq) in HCl/MeOH (5 mL, 4M), the mixture was stirred at 25° C. for 1 h. The resulting mixture was concentrated in vacuum to obtained (S)-methyl 2-((S)-2-((S)-2-amino-3-(4-fluorophenyl)propanamido)-3-cyclopropylpropanamido)-3-((R)-5,5-dimethyl-2-oxopyrrolidin-3-yl) propanoate (180 mg, crude, HCl) as a white solid. MS (ESI) m/z 491.2 [M+H]⁺

Step 3: (S)-methyl 2-((S)-3-cyclopropyl-2-((S)-3-(4-fluorophenyl)-2-(pyrazine-2-carboxamido)propanamido)propanamido)-3-((R)-5,5-dimethyl-2-oxopyrrolidin-3-yl)propanoate

To a solution of (S)-methyl 2-((S)-2-((S)-2-amino-3-(4-fluorophenyl)propanamido)-3-cyclopropyl propanamido)-3-((R)-5,5-dimethyl-2-oxopyrrolidin-3-yl) propanoate (180 mg, 341.54 umol, 1 eq, HCl) and pyrazine-2-carboxylic acid (50.86 mg, 409.85 umol, 1.2 eq) in DCM (5 mL) was added DMAP (125.18 mg, 1.02 mmol, 3 eq) and EDCI (130.95 mg, 683.08 umol, 2 eq), and the resulting mixture was stirred at 25° C. for 1 h, followed by adjustment of pH to about 1 with aq.HCl (3 mL, 1M), then was added addition water (12 mL) and was extracted with DCM (5 mL*3), then the organic phase was adjusted pH-7 with sat.NaHCO₃(5 mL), and then the organic phase was concentration in vacuum and was purified by column (SiO₂, PE:EA=1:2 to PE:EA=0:1) to obtain (S)-methyl 2-((S)-3-cyclopropyl-2-((S)-3-(4-fluorophenyl)-2-(pyrazine-2-carboxamido)propanamido)propanamido)-3-((R)-5,5-dimethyl-2-oxopyrrolidin-3-yl)propanoate (180 mg, 241.35 umol, 70.67% yield, 80% purity) as a white solid. MS (ESI) m/z 597.2 [M+H]⁺

Step 4: N—((S)-1-(((S)-1-(((S)-1-amino-3-((R)-5,5-dimethyl-2-oxopyrrolidin-3-yl)-1-oxopropan-2-yl)amino)-3-cyclopropyl-1-oxopropan-2-yl)amino)-3-(4-fluorophenyl)-1-oxopropan-2-yl)pyrazine-2-carboxamide

A solution of (S)-methyl 2-((S)-3-cyclopropyl-2-((S)-3-(4-fluorophenyl)-2-(pyrazine-2-carboxamido) propanamido) propanamido)-3-((R)-5,5-dimethyl-2-oxopyrrolidin-3-yl) propanoate (160 mg, 268.16 umol, 1 eq) in NH₃/MeOH (7M, 4 mL) was stirred at 25° C. for 16 h. The resulting mixture was concentrated in vacuum to obtain N—((S)-1-(((S)-1-(((S)-1-amino-3-((R)-5,5-dimethyl-2-oxopyrrolidin-3-yl)-1-oxopropan-2-yl)amino)-3-cyclopropyl-1-oxopropan-2-yl)amino)-3-(4-fluorophenyl)-1-oxopropan-2-yl)pyrazine-2-carboxamide (150 mg, crude) as a white solid. MS (ESI) m/z 582.3 [M+H]⁺

Step 5: N—((S)-1-(((S)-1-(((S)-1-cyano-2-((R)-5,5-dimethyl-2-oxopyrrolidin-3-yl)ethyl)amino)-3-cyclopropyl-1-oxopropan-2-yl)amino)-3-(4-fluorophenyl)-1-oxopropan-2-yl)pyrazine-2-carboxamide

To a solution of N-[(1S)-2-[[(1S)-2-[[(1S)-2-amino-1-[[(3R)-5,5-dimethyl-2-oxo-pyrrolidin-3-yl] methyl]-2-oxo-ethyl]amino]-1-(cyclopropylmethyl)-2-oxo-ethyl]amino]-1-[(4-fluorophenyl)methyl]-2-oxo-ethyl]pyrazine-2-carboxamide (140 mg, 240.70 umol, 1 eq) in DCM (5 mL) was added Burgess reagent (172.08 mg, 722.10 umol, 3 eq), and then the mixture was stirred at 25° C. for 2 h. The resulting mixture was quenched by water (0.5 mL) and was dried by blowing N₂ and was purified by prep-HPLC (column: Waters Xbridge Prep OBD C18 150*40 mm*10 um; mobile phase: [water (NH₄HCO₃)-ACN]; B %: 20%-50%, 8 min) to obtain N—((S)-1-(((S)-1-(((S)-1-cyano-2-((R)-5,5-dimethyl-2-oxopyrrolidin-3-yl)ethyl)amino)-3-cyclopropyl-1-oxopropan-2-yl)amino)-3-(4-fluorophenyl)-1-oxopropan-2-yl)pyrazine-2-carboxamide (60 mg, 106.45 umol, 44.23% yield, 100% purity) as a white solid. MS (ESI) m/z 564.2 [M+H]⁺

¹H NMR (400 MHz, DMSO-d₆) δ=9.12 (d, J=1.4 Hz, 1H), 8.95 (d, J=7.8 Hz, 1H), 8.87 (d, J=2.6 Hz, 1H), 8.70 (dd, J=1.6, 2.5 Hz, 1H), 8.58 (dd, J=7.8, 13.2 Hz, 2H), 7.84 (s, 1H), 7.25-7.19 (m, 2H), 7.05-6.98 (m, 2H), 4.96 (td, J=7.6, 8.9 Hz, 1H), 4.83 (dt, J=4.8, 8.1 Hz, 1H), 4.26 (q, J=7.2 Hz, 1H), 3.19-3.12 (m, 1H), 3.10-3.03 (m, 1H), 2.63 2.54 (m, 1H), 2.20-2.12 (m, 1H), 1.97 (dd, J=8.6, 12.3 Hz, 1H), 1.82-1.67 (m, 2H), 1.50 (dd, J=10.8, 12.0 Hz, 1H), 1.38 (td, J=7.2, 14.1 Hz, 1H), 1.16 (s, 3H), 1.05 (s, 3H), 0.78 0.67 (m, 1H), 0.46-0.35 (m, 2H), 0.17-0.02 (m, 2H).

Example 16: Synthesis of ((S)-1-(((S)-1-(((S)-1-cyano-2-((R)-5,5-dimethyl-2-oxopyrrolidin-3-yl)ethyl)amino)-3-cyclopropyl-1-oxopropan-2-yl)amino)-3-(naphthalen-1-yl)-1-oxopropan-2-yl)carbamate (Compound 126)

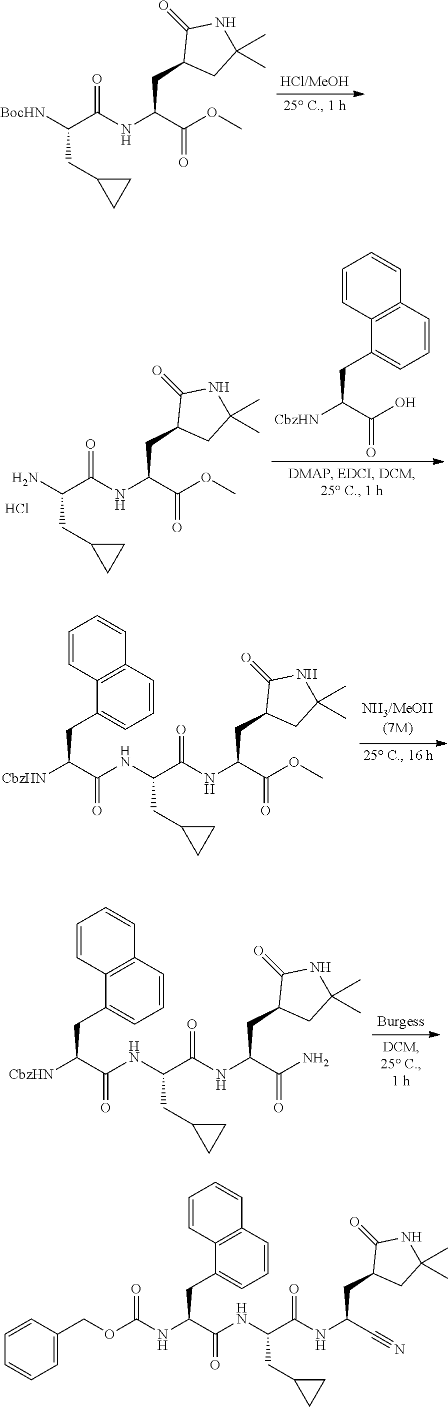

Step 1: (S)-methyl 2-((S)-2-amino-3-cyclopropylpropanamido)-3-((R)-5,5-dimethyl-2-oxopyrrolidin-3-yl)propanoate hydrochloride

To methyl (2S)-2-[[(2S)-2-(tert-butoxycarbonylamino)-3-cyclopropyl-propanoyl]amino]-3-[(3R)-5,5-dimethyl-2-oxo-pyrrolidin-3-yl]propanoate (300 mg, 690.92 umol, 98% purity, 1 eq) was added HCl/MeOH (4 M, 8 mL, 46.31 eq). The mixture was stirred at 25° C. for 1 hour. Upon completion, the reaction solution was concentrated under reduced pressure and washed with DCM twice to give methyl (2S)-2-[[(2S)-2-amino-3-cyclopropyl-propanoyl]amino]-3-[(3R)-5,5-dimethyl-2-oxo-pyrrolidin-3-yl]propanoate (330 mg, 683.96 umol, 98.99% yield, 75% purity, HCl) as a white gel. MS (ESI) m/z 326.1 [M+H]⁺.

Step 2: (5S,8S,11S)-methyl 8-(cyclopropylmethyl)-11-(((R)-5,5-dimethyl-2-oxopyrrolidin-3-yl)methyl)-5-(naphthalen-1-ylmethyl)-3,6,9-trioxo-1-phenyl-2-oxa-4,7,10-triazadodecan-12-oate

To a solution of methyl (2S)-2-[[(2S)-2-amino-3-cyclopropyl-propanoyl]amino]-3-[(3R)-5,5-dimethyl-2-oxo-pyrrolidin-3-yl]propanoate (300 mg, 829.04 umol, 1 eq, HCl) and (2S)-2-(benzyloxycarbonylamino)-3-(1-naphthyl)propanoic acid (289.65 mg, 829.04 umol, 1 eq) in DCM (5 mL) was added EDCI (317.86 mg, 1.66 mmol, 2 eq) and DMAP (303.85 mg, 2.49 mmol, 3 eq). The mixture was stirred at 25° C. for 1 hour, and then was quenched with H₂O (10 mL), extracted with DCM 30 mL (10 mL*3). The organic layers were combined and washed with citric acid (10 mL), NaHCO₃aq (10 mL), brine (10 mL), dried over Na₂SO₄ and concentrated under reduced pressure to give a residue. The residue was purified by column chromatography (SiO₂, DCM:MeOH=10:1) to give methyl (2S)-2-[[(2S)-2-[[(2S)-2-(benzyloxycarbonylamino)-3-(1-naphthyl)propanoyl]amino]-3-cyclopropyl-propanoyl]amino]-3-[(3R)-5,5-dimethyl-2-oxo-pyrrolidin-3-yl]propanoate (310 mg, 445.58 umol, 53.75% yield, 94.4% purity) as a white solid. MS (ESI) m/z 657.5 [M+H]⁺.

Step 3: Benzyl ((S)-1-(((S)-1-(((S)-1-amino-3-((R)-5,5-dimethyl-2-oxopyrrolidin-3-yl)-1-oxopropan-2-yl)amino)-3-cyclopropyl-1-oxopropan-2-yl)amino)-3-(naphthalen-1-yl)-1-oxopropan-2-yl)carbamate

To methyl (2S)-2-[[(2S)-2-[[(2S)-2-(benzyloxycarbonylamino)-3-(1-naphthyl)propanoyl]amino]-3-cyclopropyl-propanoyl]amino]-3-[(3R)-5,5-dimethyl-2-oxo-pyrrolidin-3-yl]propanoate (300 mg, 456.78 umol, 1 eq) was added NH₃/MeOH (7 M, 10 mL, 153.25 eq). The mixture was stirred at 25° C. for 16 hours. Upon completion, the reaction residue was concentrated under reduced pressure, and washed with DCM twice to give benzyl N-[(1S)-2-[[(1S)-2-[[(1S)-2-amino-1-[[(3R)-5,5-dimethyl-2-oxo-pyrrolidin-3-yl]methyl]-2-oxo-ethyl]amino]-1-(cyclopropylmethyl)-2-oxo-ethyl]amino]-1-(1-naphthylmethyl)-2-oxo-ethyl]carbamate (250 mg, crude) as a white solid. MS (ESI) m/z 642.3 [M+H]⁺.

Step 4: Benzyl ((S)-1-(((S)-1-(((S)-1-cyano-2-((R)-5,5-dimethyl-2-oxopyrrolidin-3-yl)ethyl)amino)-3-cyclopropyl-1-oxopropan-2-yl)amino)-3-(naphthalen-1-yl)-1-oxopropan-2-yl)carbamate

To a solution of benzyl N-[(1S)-2-[[(1S)-2-[[(1S)-2-amino-1-[[(3R)-5,5-dimethyl-2-oxo-pyrrolidin-3-yl]methyl]-2-oxo-ethyl]amino]-1-(cyclopropylmethyl)-2-oxo-ethyl]amino]-1-(1-naphthylmethyl)-2-oxo-ethyl]carbamate (250 mg, 389.56 umol, 1 eq) in DCM (10 mL) was added Burgess reagent (278.51 mg, 1.17 mmol, 3 eq). The mixture was stirred at 25° C. for 1 hour. Upon completion, the reaction was quenched with H₂O (5 mL).

The reaction solution was dried over N₂. The residue was purified by prep-HPLC (FA condition: column: Phenomenex Luna C18 200*40 mm*10 um; mobile phase: [water(FA)-ACN]; B %: 40%-80%, 8 min) to give benzyl N-[(1S)-2-[[(1S)-2-[[(1S)-1-cyano-2-[(3R)-5,5-dimethyl-2-oxo-pyrrolidin-3-yl]ethyl]amino]-1-(cyclopropylmethyl)-2-oxo-ethyl]amino]-1-(1-naphthylmethyl)-2-oxo-ethyl]carbamate (100 mg, 160.32 umol, 41.16% yield, 100% purity) as a white solid. MS (ESI) m/z 624.2 [M+H]⁺.

¹H NMR (400 MHz, DMSO-d₆) δ=8.87 (br d, J=7.8 Hz, 1H), 8.23 (br d, J=7.5 Hz, 1H), 8.18 (br d, J=8.1 Hz, 1H), 7.92 (br d, J=7.4 Hz, 1H), 7.85 (s, 1H), 7.82-7.74 (m, 1H), 7.65-7.57 (m, 1H), 7.57-7.47 (m, 2H), 7.41 (q, J=7.0 Hz, 2H), 7.35-7.24 (m, 3H), 7.17 (br d, J=6.4 Hz, 2H), 5.01-4.92 (m, 1H), 4.89 (s, 2H), 4.45 (dt, J=3.8, 9.4 Hz, 1H), 4.31 (q, J=7.2 Hz, 1H), 3.65-3.49 (m, 1H), 3.15 (br dd, J=10.3, 14.0 Hz, 1H), 2.64-2.54 (m, 1H), 2.23-2.12 (m, 1H), 2.03 (br dd, J=8.5, 12.0 Hz, 1H), 1.83-1.75 (m, 1H), 1.70 (td, Jβ0.7, 13.7 Hz, 1H), 1.53 (br t, J=11.5 Hz, 1H), 1.41 (td, J=6.9, 14.1 Hz, 1H), 1.17 (s, 3H), 1.13-1.08 (m, 3H), 0.72 (br d, J=6.0 Hz, 1H), 0.41 (br d, J=7.0 Hz, 2H), 0.19-0.02 (m, 2H).

Example 17: Synthesis of Benzyl ((2S)-1-((1-(((S)-1-cyano-2-((R)-5,5-dimethyl-2-oxopyrrolidin-3-yl)ethyl)amino)-3-(4,4-difluorocyclohexyl)-1-oxopropan-2-yl)amino)-3-(naphthalen-1-yl)-1-oxopropan-2-yl)carbamate (Compound 127)

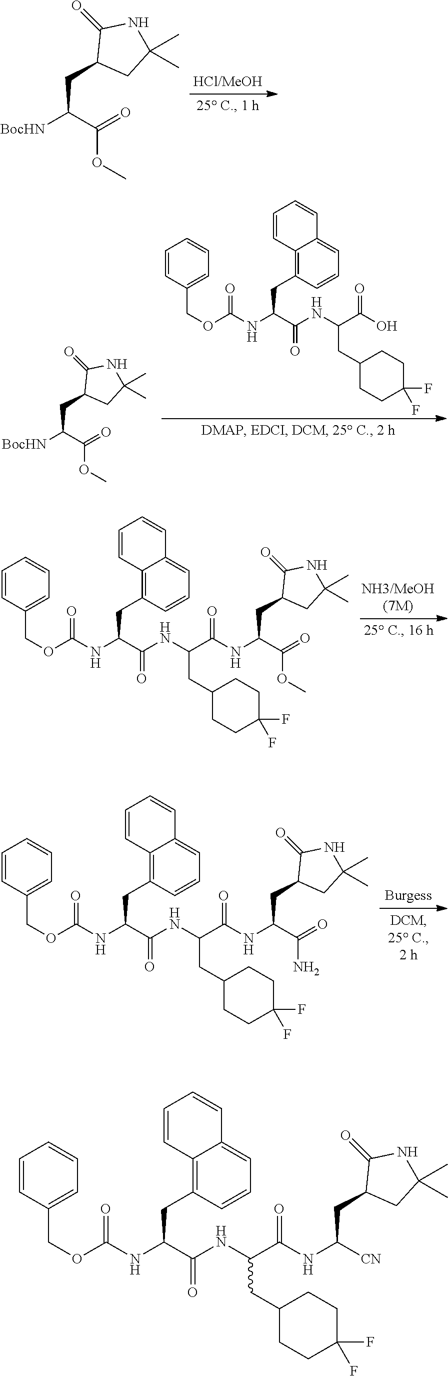 [SMILES: COC(=O)[C@H](C[C@@H]1CC(C)(C)NC1=O)NC(=O)OC(C)(C)C ; COC(=O)[C@H](C[C@@H]1CC(C)(C)NC1=O)NC(=O)OC(C)(C)C ; O=C(N[C@@H](Cc1cccc2ccccc12)C(=O)NC(CC1CCC(F)(F)CC1)C(=O)O)OCc1ccccc1 ; COC(=O)[C@H](C[C@@H]1CC(C)(C)NC1=O)NC(=O)C(CC1CCC(F)(F)CC1)NC(=O)[C@H](Cc1cccc2ccccc12)NC(=O)OCc1ccccc1 ; CC1(C)C[C@@H](C[C@H](NC(=O)C(CC2CCC(F)(F)CC2)NC(=O)[C@H](Cc2cccc3ccccc23)NC(=O)OCc2ccccc2)C(N)=O)C(=O)N1 ; CC1(C)C[C@@H](C[C@@H](C#N)NC(=O)C(CC2CCC(F)(F)CC2)NC(=O)[C@H](Cc2cccc3ccccc23)NC(=O)OCc2ccccc2)C(=O)N1]

Step 1: methyl (2S)-2-amino-3-[(3R)-5,5-dimethyl-2-oxo-pyrrolidin-3-yl]propanoate

A solution of methyl (2S)-2-(tert-butoxycarbonylamino)-3-[(3R)-5,5-dimethyl-2-oxo-pyrrolidin-3-yl]propanoate (1.1 g, 3.50 mmol, 1 eq) in HCl/MeOH (7 M, 20 mL, 40.01 eq) was stirred at 25° C. for 1 h. Upon completion, the reaction mixture was concentrated under reduced pressure to give methyl (2S)-2-amino-3-[(3R)-5,5-dimethyl-2-oxo-pyrrolidin-3-yl]propanoate (700 mg, 3.27 mmol, 93.37% yield) as a white solid. MS (ESI) m/z 215.0 [M+H]⁺.

Step 2: methyl (2S)-2-[[2-[[(2S)-2-(benzyloxycarbonylamino)-3-(1-naphthyl)propanoyl]amino]-3-(4,4-difluorocyclohexyl)propanoyl]amino]-3-[(3R)-5,5-dimethyl-2-oxo-pyrrolidin-3-yl]propanoate

To a solution of 2-[[(2S)-2-(benzyloxycarbonylamino)-3-(1-naphthyl)propanoyl]amino]-3-(4,4-difluorocyclohexyl)propanoic acid (300.00 mg, 557.02 umol, 1 eq) and methyl (2S)-2-amino-3-[(3R)-5,5-dimethyl-2-oxo-pyrrolidin-3-yl]propanoate (119.35 mg, 557.02 umol, 1 eq) in DCM (10 mL) was added EDCI (213.56 mg, 1.11 mmol, 2 eq) and DMAP (204.15 mg, 1.67 mmol, 3 eq). The mixture was stirred at 25° C. for 2 h. Upon completion, the reaction solution was quenched with H₂O (20 mL), extracted with DCM 60 mL (20 mL*3). The organic layers were combined, washed with citric acid (20 mL), NaHCO₃aq. (20 mL), brine (20 mL), dried over Na₂SO₄ and concentrated under reduced pressure to give a residue. The residue was purified by column chromatography (SiO₂, DCM:MeOH=10:1) to give methyl (2S)-2-[[2-[[(2S)-2-(benzyloxycarbonylamino)-3-(1-naphthyl)propanoyl]amino]-3-(4,4-difluorocyclohexyl)propanoyl]amino]-3-[(3R)-5,5-dimethyl-2-oxo-pyrrolidin-3-yl]propanoate (250 mg, 340.22 umol, 61.08% yield) as a white solid. MS (ESI) m/z 735.5 [M+H]⁺.

Step 3: benzyl N-[(1S)-2-[[(1S)-2-[[(1S)-2-amino-1-[[(3R)-5,5-dimethyl-2-oxo-pyrrolidin-3-yl]methyl]-2-oxo-ethyl]amino]-1-[(4,4-difluorocyclohexyl)methyl]-2-oxo-ethyl]amino]-1-(1-naphthylmethyl)-2-oxo-ethyl]carbamate

A solution of methyl (2S)-2-[[(2S)-2-[[(2S)-2-(benzyloxycarbonylamino)-3-(1-naphthyl)propanoyl]amino]-3-(4,4-difluorocyclohexyl)propanoyl]amino]-3-[(3R)-5,5-dimethyl-2-oxo-pyrrolidin-3-yl]propanoate (240 mg, 326.61 umol, 1 eq) in NH₃/MeOH (7 M, 10 mL, 214.32 eq) was stirred at 25° C. for 16 h. Upon completion, the reaction solution was concentrated under reduced pressure to give benzyl N-[(1S)-2-[[(1S)-2-[[(1S)-2-amino-1 [[(3R)-5,5-dimethyl-2-oxo-pyrrolidin-3-yl]methyl]-2-oxo-ethyl]amino]-1-[(4,4-difluorocyclohexyl)methyl]-2-oxo-ethyl]amino]-1-(1-naphthylmethyl)-2-oxo-ethyl]carbamate (200 mg, 277.85 umol, 85.07% yield) as a white solid. MS (ESI) m/z 720.5 [M+H]⁺.

Step 4: benzyl N-[(1S)-2-[[2-[[(1S)-1-cyano-2-[(3R)-5,5-dimethyl-2-oxo-pyrrolidin-3-yl]ethyl]amino]-1-[(4,4-difluorocyclohexyl)methyl]-2-oxo-ethyl]amino]-1-(1-naphthylmethyl)-2-oxo-ethyl]carbamate

To a solution of benzyl N-[(1S)-2-[[(1S)-2-[[(1S)-2-amino-1-[[(3R)-5,5-dimethyl-2-oxo-pyrrolidin-3-yl]methyl]-2-oxo-ethyl]amino]-1-[(4,4-difluorocyclohexyl)methyl]-2-oxo-ethyl]amino]-1-(1-naphthylmethyl)-2-oxo-ethyl]carbamate (200 mg, 277.85 umol, 1 eq) in DCM (5 mL) was added Burgess reagent (198.64 mg, 833.55 umol, 3 eq). The mixture was stirred at 25° C. for 2 h. Upon completion, the reaction mixture was quenched by H₂O (20 mL). The organic phase was separated, washed with DCM 60 mL (20 mL*3), dried over Na₂SO₄, filtered and concentrated under reduced pressure to give a residue. The residue was purified by prep-TLC (SiO₂, DCM:MeOH=10:1). The product was further seperated by SFC (REGIS(S,S)WHELK-O1(250 mm*25 mm, 10 um); mobile phase: [Neu-IPA]; B %: 40%-40%, 15 min) to give benzyl N-[(1S)-2-[[2-[[(1S)-1-cyano-2-[(3R)-5,5-dimethyl-2-oxo-pyrrolidin-3-yl]ethyl]amino]-1-[(4,4-difluorocyclohexyl)methyl]-2-oxo-ethyl]amino]-1-(1-naphthylmethyl)-2-oxo-ethyl]carbamate (Isomer 1) (22 mg, 31.19 umol, 11.23% yield, 99.5% purity) and benzyl N-[(1S)-2-[[2-[[(1S)-1-cyano-2-[(3R)-5,5-dimethyl-2-oxo-pyrrolidin-3-yl]ethyl]amino]-1-[(4,4-difluorocyclohexyl)methyl]-2-oxo-ethyl]amino]-1-(1-naphthylmethyl)-2-oxo-ethyl]carbamate (Isomer 2) (35 mg, 48.13 umol, 17.32% yield, 96.5% purity) as white solids. MS (ESI) m/z 702.2 [M+H]⁺.

Isomer 1: ¹H NMR (400 MHz, DMSO-d₆) δ=8.85 (br d, J=8.0 Hz, 1H), 8.30 (br d, J=7.9 Hz, 1H), 8.17 (br d, J=8.3 Hz, 1H), 7.92 (br d, J=7.8 Hz, 1H), 7.86 (s, 1H), 7.80 (br d, J=7.5 Hz, 1H), 7.66-7.49 (m, 3H), 7.47-7.37 (m, 2H), 7.36-7.24 (m, 3H), 7.19 (brd, J=6.5 Hz, 2H), 4.98-4.86 (m, 3H), 4.48-4.40 (m, 1H), 4.39-4.31 (m, 1H), 3.53 (br dd, J=3.8, 14.2 Hz, 1H), 3.16 (br dd, J=10.2, 14.3 Hz, 1H), 2.23-2.13 (m, 1H), 2.07-1.92 (m, 3H), 1.84-1.70 (m, 4H), 1.68-1.39 (m, 6H), 1.23 (br s, 2H), 1.17 (s, 3H), 1.11 (s, 3H).

Isomer 2: ¹H NMR (400 MHz, DMSO-d₆) δ=8.68 (br d, J=8.0 Hz, 1H), 8.37 (br d, J=8.0 Hz, 1H), 8.18 (d, J=8.4 Hz, 1H), 7.96-7.85 (m, 2H), 7.84-7.69 (m, 2H), 7.62-7.48 (m, 2H), 7.41 (q, J=7.0 Hz, 2H), 7.37-7.19 (m, 5H), 5.03-4.90 (m, 3H), 4.46 (q, J=7.8 Hz, 1H), 4.29-4.17 (m, 1H), 3.47 (br dd, J=6.4, 13.9 Hz, 1H), 3.24 (br dd, J=8.6, 13.8 Hz, 1H), 2.23-2.11 (m, 1H), 2.01 (br dd, J=8.8, 11.9 Hz, 1H), 1.90 (br s, 2H), 1.84-1.72 (m, 1H), 1.65-1.36 (m, 7H), 1.23 (br s, 1H), 1.18 (s, 3H), 1.12 (s, 3H), 1.06 (br s, 3H).

Example 18: Synthesis of (1R,2S,5S)—N-[(1S)-1-cyan-2-[(6R)-5-oxo-4-azaspiro[2.4]heptan-6-yl]ethyl]-3-[(2S)-3,3-dimethyl-2-(pyrazine-2-carbonylamino)butanoyl]-6,6-dimethyl-3-azabicyclo[3.1.0]hexane-2-carboxamide (Compound 129)

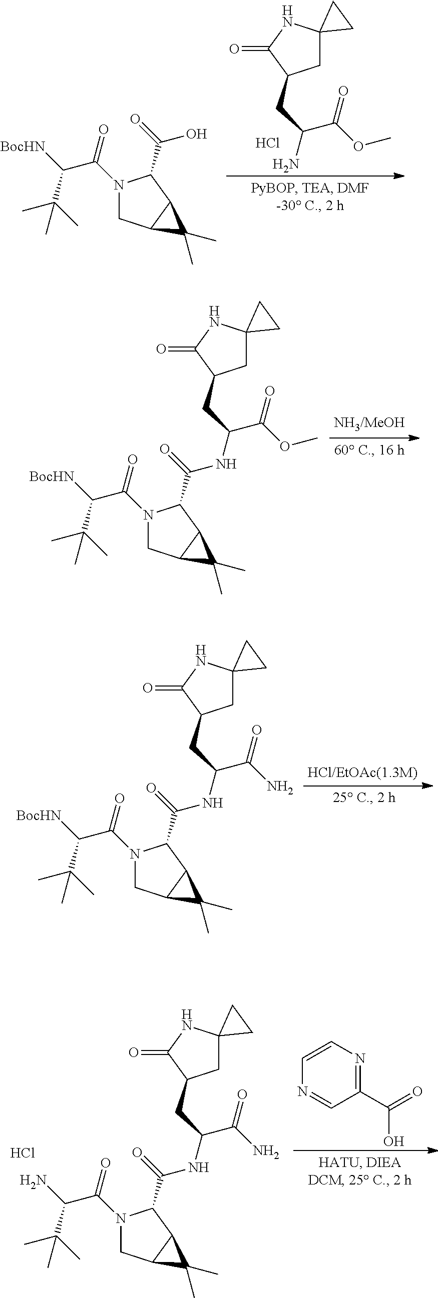

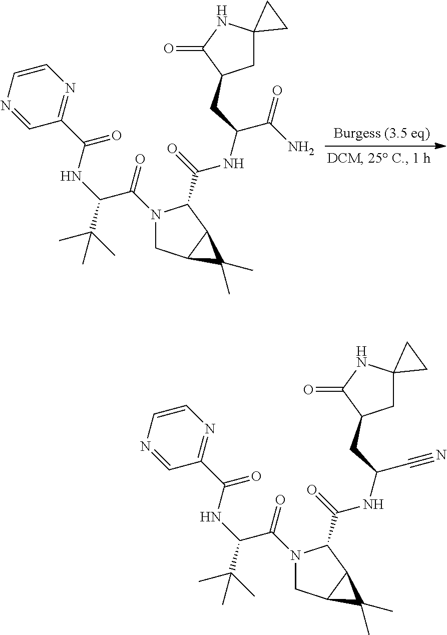 [SMILES: CC(C)(C)[C@H](NC(=O)c1cnccn1)C(=O)N1C[C@H]2[C@@H]([C@H]1C(=O)N[C@@H](C[C@@H]1CC3(CC3)NC1=O)C(N)=O)C2(C)C ; CC(C)(C)[C@H](NC(=O)c1cnccn1)C(=O)N1C[C@H]2[C@@H]([C@H]1C(=O)N[C@H](C#N)C[C@@H]1CC3(CC3)NC1=O)C2(C)C]

Step 1: methyl (2S)-2-[[(1R,2S,5S)-3-[(2S)-2-(tert-butoxycarbonylamino)-3,3-dimethyl-butanoyl]-6,6-dimethyl-3-azabicyclo[3.1.0]hexane-2-carbonyl]amino]-3-[(6R)-5-oxo-4-azaspiro[2.4]heptan-6-yl]propanoate

To a mixture of (1R,2S,5S)-3-[(2S)-2-(tert-butoxycarbonylamino)-3,3-dimethyl-butanoyl]-6,6-dimethyl-3-azabicyclo[3.1.0]hexane-2-carboxylic acid (200 mg, 542.79 umol, 1 eq), methyl (2S)-2-amino-3-[(6R)-5-oxo-4-azaspiro[2.4]heptan-6-yl]propanoate (161.99 mg, 651.35 umol, 1.2 eq, HCl) and PyBOP (310.71 mg, 597.07 umol, 1.1 eq) in DMF (2 mL) at −30° C. was added TEA (109.85 mg, 1.09 mmol, 151.10 uL, 2 eq). After the addition, the reaction mixture was stirred at −30° C. for 2 h. Upon completion, the reaction mixture was diluted by water (10 mL), extracted with EtOAc (5 mL*2). The combined organic layers were washed with brine (5 mL*3), dried over Na₂SO₄, filtered, concentrated in a vacuum to afford crude product. The crude product was purified by MPLC (100-200 mesh silica gel, EtOAc/MeOH=I/O to EtOAc/MeOH=10/1) to afford methyl (2S)-2-[[(1R,2S,5S)-3-[(2S)-2-(tert-butoxycarbonylamino)-3,3-dimethyl-butanoyl]-6,6-dimethyl-3-azabicyclo[3.1.0]hexane-2-carbonyl]amino]-3-[(6R)-5-oxo-4-azaspiro[2.4]heptan-6-yl]propanoate (400 mg, crude) as yellow oil. MS (ESI) m/z 563.4 [M+H]⁺.

Step 2: tert-butyl N-[(1S)-1-[(1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl-propyl]carbamate

A mixture of methyl (2S)-2-[[(1R,2S,5S)-3-[(2S)-2-(tert-butoxycarbonylamino)-3,3-dimethyl-butanoyl]-6,6-dimethyl-3-azabicyclo[3.1.0]hexane-2-carbonyl]amino]-3-[(6R)-5-oxo-4-azaspiro[2.4]heptan-6-yl]propanoate (290 mg, 515.38 umol, 1 eq) in NH₃/MeOH (7 M, 7.44 mL, 101.00 eq) was stirred at 65° C. for 16 h. Upon completion, the reaction mixture was concentrated in a vacuum to dryness to afford tert-butyl N-[(1S)-1-[(1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl-propyl]carbamate (233 mg, crude) as yellow solid, which was used for next step without further purification. MS (ESI) m/z 548.4 [M+H]⁺.

Step 3: (1R,2S,5S)-3-[(2S)-2-amino-3,3-dimethyl-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide

A mixture of tert-butyl N-[(1S)-1-[(1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl-propyl]carbamate (223 mg, 407.17 umol, 1 eq) in HCl/EtOAc (1.3 M, 11.74 mL, 37.47 eq) was stirred at 25° C. for 4 h. Upon completion, the reaction mixture was concentrated in a vacuum to dryness to afford (1R,2S,5S)-3-[(2S)-2-amino-3,3-dimethyl-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide (160 mg, crude, HCl) as white solid, which was used for next step without further purification. MS (ESI) m/z 448.3 [M+H]⁺.

Step 4: (1R,2S,5S)—N-[(1S)-2-amino-2-oxo-1-[[(6R)-5-oxo-4-azaspiro[2.4]heptan-6-yl]methyl]ethyl]-3-[(2S)-3,3-dimethyl-2-(pyrazine-2-carbonylamino)butanoyl]-6,6-dimethyl-3-azabicyclo[3.1.0]hexane-2-carboxamide

To a solution of pyrazine-2-carboxylic acid (49.23 mg, 396.67 umol, 1.2 eq) in DCM (20 mL) was added HATU (188.53 mg, 495.84 umol, 1.5 eq), DIEA (128.17 mg, 991.67 umol, 172.73 uL, 3 eq) and (1R,2S,5S)-3-[(2S)-2-amino-3,3-dimethyl-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide (160 mg, 330.56 umol, 1 eq, HCl) in turn. The reaction mixture was stirred at 25° C. for 2 hr. Upon completion, the reaction mixture was quenched by water (50 mL). The resulting mixture was extracted with DCM (20 mL*3). The combined organic layers were dried over Na₂SO₄, filtered, concentrated in a vacuum to give crude product. The crude product was purified by prep-TLC to afford (1R,2S,5S)—N-[(1S)-2-amino-2-oxo-1-[[(6R)-5-oxo-4-azaspiro[2.4]heptan-6-yl]methyl]ethyl]-3-[(2S)-3,3-dimethyl-2-(pyrazine-2-carbonylamino)butanoyl]-6,6-dimethyl-3-azabicyclo[3.1.0]hexane-2-carboxamide (73 mg, 64.61 umol, 19.54% yield, 49% purity) as white solid. MS (ESI) m/z 554.2 [M+H]⁺.

Step 5: (1R,2S,5S)—N-[(1S)-1-cyano-2-[(6R)-5-oxo-4-azaspiro[2.4]heptan-6-yl]ethyl]-3-[(2S)-3,3-dimethyl-2-(pyrazine-2-carbonylamino)butanoyl]-6,6-dimethyl-3-azabicyclo[3.1.0]hexane-2-carboxamide

To a solution of (1R,2S,5S)—N-[(1S)-2-amino-2-oxo-1-[[(6R)-5-oxo-4-azaspiro[2.4]heptan-6-yl]methyl]ethyl]-3-[(2S)-3,3-dimethyl-2-(pyrazine-2-carbonylamino)butanoyl]-6,6-dimethyl-3-azabicyclo[3.1.0]hexane-2-carboxamide (73 mg, 131.85 umol, 1 eq) in DCM (5 mL) was added Burgess reagent (109.97 mg, 461.48 umol, 3.5 eq). The resulting mixture was stirred at 25° C. for 3 h. Upon completion, the reaction mixture was blowed to dryness with N₂ and then diluted with DMF (1.5 mL). The resulting mixture was purified by prep-HPLC (column: Waters Xbridge BEH C18 100*30 mm*10 um; mobile phase: [water (NH₄HCO₃)-ACN]; B %: 20%-50%, 10 min) to afford (1R,2S,5S)—N-[(1S)-1-cyano-2-[(6R)-5-oxo-4-azaspiro[2.4]heptan-6-yl]ethyl]-3-[(2S)-3,3-dimethyl-2-(pyrazine-2-carbonylamino)butanoyl]-6,6-dimethyl-3-azabicyclo[3.1.0]hexane-2-carboxamide (14 mg, 26.14 umol, 19.82% yield, 100% purity) as white solid. MS (ESI) m/z 536.2 [M+H]⁺

¹H NMR (400 MHz, METHANOL-d4) δ=6.90 (d, J=3.9 Hz, 1H), 6.58 (d, J=3.9 Hz, 1H), 5.10-5.00 (m, 1H), 4.62-4.50 (m, 1H), 2.86 (tq, J=4.8, 9.3 Hz, 1H), 2.43 2.30 (m, 1H), 2.15-1.88 (m, 5H), 1.77-1.60 (m, 1H), 1.50 (ddt, J=4.4, 7.7, 11.9 Hz, 1H), 1.19-1.03 (m, 1H), 0.88-0.79 (m, 1H), 0.73-0.65 (m, 1H), 0.65-0.53 (m, 2H)

¹H NMR (400 MHz, DMSO-d₆) δ=9.22-9.16 (m, 1H), 9.06 (d, J=8.5 Hz, 1H), 8.94-8.89 (m, 1H), 8.75 (dd, J=1.5, 2.4 Hz, 1H), 8.27-8.07 (m, 1H), 7.92-7.73 (m, 1H), 5.00-4.80 (m, 1H), 4.59 (d, J=9.4 Hz, 1H), 4.22-4.14 (m, 1H), 3.97 (dd, J=5.5, 10.4 Hz, 1H), 3.80 (d, J=10.5 Hz, 1H), 2.66-2.59 (m, 1H), 2.19 (ddd, J=4.5, 10.6, 13.5 Hz, 1H), 2.04-1.90 (m, 2H), 1.82 (ddd, J=5.4, 10.7, 13.5 Hz, 1H), 1.63-1.47 (m, 1H), 1.43-1.31 (m, 1H), 1.03 (s, 3H), 1.01 (s, 9H), 0.82 (s, 3H), 0.78-0.71 (m, 1H), 0.64-0.49 (m, 3H)

Example 19: Synthesis of benzyl ((S)-1-(((S)-1-(((S)-1-cyano-2-((S)-2-oxopiperidin-3-yl)ethyl)amino)-3-cyclopropyl-1-oxopropan-2-yl amino)-3-(4-fluorophenyl)-1-oxopropan-2-yl)carbamate (Compound 130)

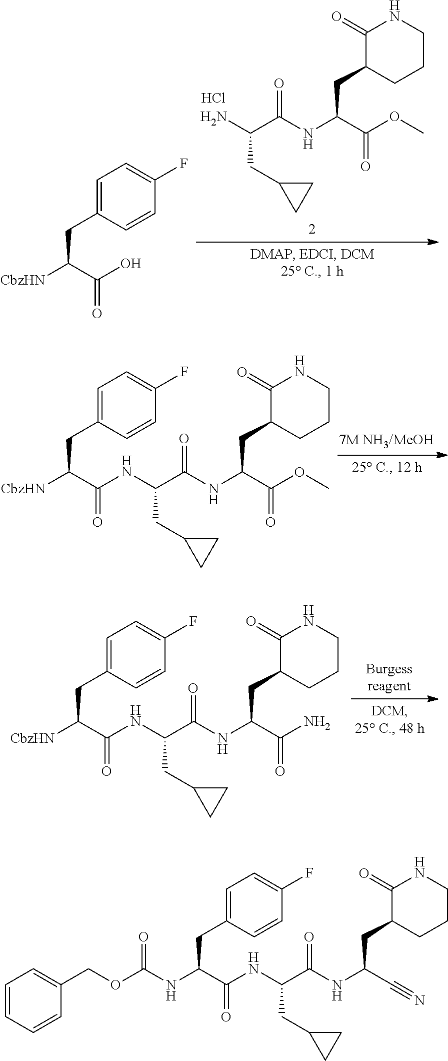 [SMILES: O=C(N[C@@H](Cc1ccc(F)cc1)C(=O)O)OCc1ccccc1 ; Cl ; COC(=O)[C@H](C[C@@H]1CCCNC1=O)NC(=O)[C@@H](N)CC1CC1 ; COC(=O)[C@H](C[C@@H]1CCCNC1=O)NC(=O)[C@H](CC1CC1)NC(=O)[C@H](Cc1ccc(F)cc1)NC(=O)OCc1ccccc1 ; NC(=O)[C@H](C[C@@H]1CCCNC1=O)NC(=O)[C@H](CC1CC1)NC(=O)[C@H](Cc1ccc(F)cc1)NC(=O)OCc1ccccc1 ; N#C[C@H](C[C@@H]1CCCNC1=O)NC(=O)[C@H](CC1CC1)NC(=O)[C@H](Cc1ccc(F)cc1)NC(=O)OCc1ccccc1]

Step 1: (5S,8S,11S)-methyl 8-(cyclopropylmethyl)-5-(4-fluorobenzyl)-3,6,9-trioxo-11-(((S)-2-oxopiperidin-3-yl)methyl)-1-phenyl-2-oxa-4,7,10-triazadodecan-12-oate

To a solution of (2S)-2-(benzyloxycarbonylamino)-3-(4-fluorophenyl)propanoic acid (500 mg, 1.58 mmol, 1 eq) and methyl (2S)-2-[[(2S)-2-amino-3-cyclopropyl-propanoyl]amino]-3-[(3S)-2-oxo-3-piperidyl]propanoate (588.78 mg, 1.89 mmol, 1.2 eq) in DCM (8 mL) was added DMAP (577.52 mg, 4.73 mmol, 3 eq) and EDCI (604.15 mg, 3.15 mmol, 2 eq). The resulting mixture was stirred at 25° C. for 1 h. Upon completion, the reaction mixture was diluted with H₂O (60 mL) and extracted with DCM 120 mL (40 mL*3). The combined organic layers were washed with brine (50 mL), dried over Na₂SO₄, filtered and concentrated under reduced pressure to give a residue. The residue was purified by prep-TLC (SiO₂, DCM: MeOH=10:1) to give methyl (2S)-2-[[(2S)-2-[[(2S)-2-(benzyloxycarbonylamino)-3-(4-fluorophenyl)propanoyl]amino]-3-cyclopropyl-propanoyl]amino]-3-[(3S)-2-oxo-3-piperidyl]propanoate (500 mg, 818.77 umol, 51.96% yield) as a white solid. MS (ESI) m/z 611.3 [M+H]⁺.

Step 2: benzyl ((S)-1-(((S)-1-(((S)-1-amino-1-oxo-3-((S)-2-oxopiperidin-3-yl)propan-2-yl)amino)-3-cyclopropyl-1-oxopropan-2-yl)amino)-3-(4-fluorophenyl)-1-oxopropan-2-yl)carbamate

A solution of methyl (2S)-2-[[(2S)-2-[[(2S)-2-(benzyloxycarbonylamino)-3-(4-fluorophenyl)propanoyl]amino]-3-cyclopropyl-propanoyl]amino]-3-[(3S)-2-oxo-3-piperidyl]propanoate (150 mg, 245.63 umol, 1 eq) in NH₃/MeOH (7 M, 10 mL, 284.98 eq) was stirred at 25° C. for 12 h. Upon completion, the reaction mixture was concentrated under reduced pressure to give benzyl N-[(1S)-2-[[(1S)-2-[[(1S)-2-amino-2-oxo-1-[[(3S)-2-oxo-3-piperidyl]methyl]ethyl]amino]-1-(cyclopropylmethyl)-2-oxo-ethyl]amino]-1-[(4-fluorophenyl)methyl]-2-oxo-ethyl]carbamate (140 mg, crude) as a white solid. MS (ESI) m/z 596.2 [M+H]⁺.

Step 3: benzyl ((S)-1-(((S)-1-(((S)-1-cyano-2-((S)-2-oxopiperidin-3-yl)ethyl)amino)-3-cyclopropyl-1-oxopropan-2-yl)amino)-3-(4-fluorophenyl)-1-oxopropan-2-yl)carbamate

To a solution of benzyl N-[(1S)-2-[[(1S)-2-[[(1S)-2-amino-2-oxo-1-[[(3S)-2-oxo-3-piperidyl]methyl]ethyl]amino]-1-(cyclopropylmethyl)-2-oxo-ethyl]amino]-1-[(4-fluorophenyl)methyl]-2-oxo-ethyl]carbamate (140 mg, 235.03 umol, 1 eq) in DCM (3 mL) was added Burgess reagent (504.09 mg, 2.12 mmol, 9 eq). The mixture was stirred at 25° C. for 2 h. Upon completion, the reaction mixture was concentrated under reduced pressure to remove solvent by N₂. The residue was purified by prep-HPLC (column: Waters Xbridge BEH C18 100*30 mm*loum; mobile phase: [water(NH₃H₂O+NH₄HCO₃)-ACN]; B %: 25%-55%, 8 min) to give benzyl N-[(1S)-2-[[(1S)-2-[[(1S)-1-cyano-2-[(3S)-2-oxo-3-piperidyl]ethyl]amino]-1-(cyclopropylmethyl)-2-oxo-ethyl]amino]-1-[(4-fluorophenyl)methyl]-2-oxo-ethyl]carbamate (40 mg, 69.25 umol, 29.46% yield, 100% purity) as a white solid. MS (ESI) m/z 578.2 [M+H]⁺.

¹H NMR (400 MHz, DMSO-d₆) δ=8.86 (d, J=7.6 Hz, 1H), 8.24 (d, J=7.3 Hz, 1H), 7.62-7.43 (m, 2H), 7.41-7.19 (m, 7H), 7.12-7.01 (m, 2H), 5.05 (d, J=7.8 Hz, 1H), 4.93 (s, 2H), 4.36-4.19 (m, 2H), 3.17-3.04 (m, 2H), 2.98 (dd, J=3.1, 13.7 Hz, 1H), 2.69 (dd, J=11.1, 12.9 Hz, 1H), 2.30-2.16 (m, 2H), 1.90-1.56 (m, 5H), 1.38 (dd, J=7.0, 13.5 Hz, 2H), 0.71 (dd, J=5.9, 7.1 Hz, 1H), 0.40 (d, J=6.5 Hz, 2H), 0.21-0.01 (m, 2H)

Example 20: Synthesis of benzylN-[(1S)-2-[[2-[[(1S)-1-cyano-2-[(3S)-2-oxopyrrolidin-3-yl]ethyl]amino]-1-[(4,4-difluorocyclohexyl)methyl]-2-oxo-ethyl]amino]-1-(1-naphthylmethyl)-2-oxo-ethyl]carbamate (Compound 128)

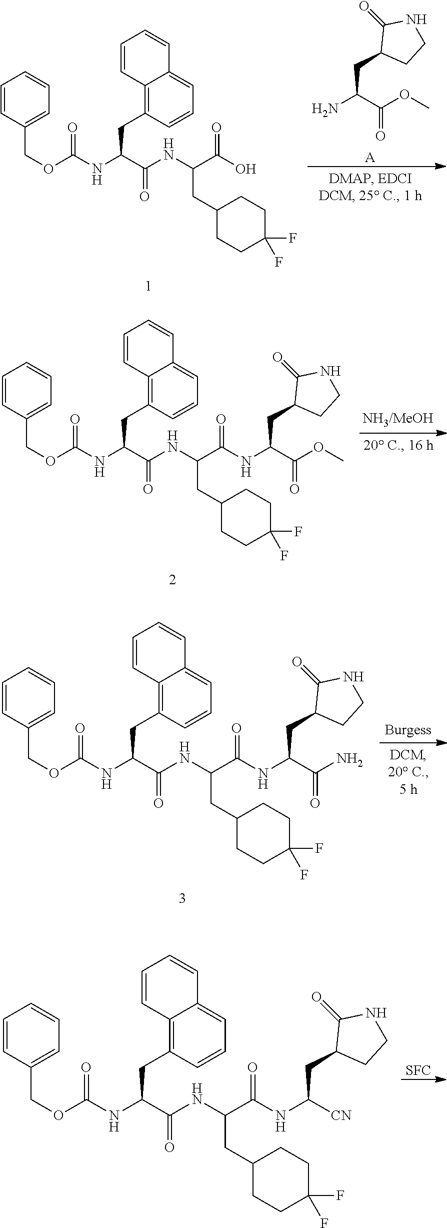

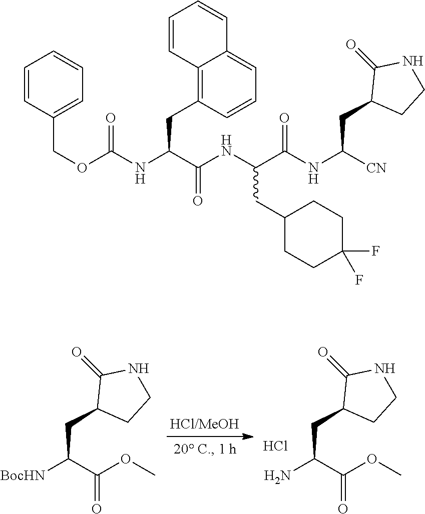 [SMILES: N#C[C@H](C[C@@H]1CCNC1=O)NC(=O)C(CC1CCC(F)(F)CC1)NC(=O)[C@H](Cc1cccc2ccccc12)NC(=O)OCc1ccccc1 ; COC(=O)[C@H](C[C@@H]1CCNC1=O)NC(=O)OC(C)(C)C ; Cl ; COC(=O)[C@@H](N)C[C@@H]1CCNC1=O]

Step 1: methyl (2S)-2-amino-3-[(3S)-2-oxopyrrolidin-3-yl]propanoate

A mixture of methyl (2S)-2-(tert-butoxycarbonylamino)-3-[(3S)-2-oxopyrrolidin-3-yl]propanoate (1 g, 3.49 mmol, 1 eq) in HCl/MeOH (4 M, 10 mL, 11.45 eq) was stirred at 20° C. for 1 h. Upon completion, the mixture was concentrated under the reduced pressure to give a the product methyl (2S)-2-amino-3-[(3S)-2-oxopyrrolidin-3-yl]propanoate (600 mg, crude, HCl) as a yellow solid.

Step 2: methyl(2S)-2-[[2-[[(2S)-2-(benzyloxycarbonylamino)-3-(1-naphthyl)propanoyl]amino]-3-(4,4-difluorocyclohexyl)propanoyl]amino]-3-[(3S)-2-oxopyrrolidin-3-yl]propanoate

To a solution of 2-[[(2S)-2-(benzyloxycarbonylamino)-3-(1-naphthyl)propanoyl]amino]-3-(4,4-difluorocyclohexyl)propanoic acid (250.00 mg, 464.18 umol, 1 eq) and methyl (2S)-2-amino-3-[(3S)-2-oxopyrrolidin-3-yl]propanoate (124.03 mg, 557.02 umol, 1.2 eq, HCl) in DCM (10 mL) was added DMAP (113.42 mg, 928.37 umol, 2 eq) and EDCI (177.97 mg, 928.37 umol, 2 eq). The mixture was stirred at 20° C. for 1 h.

Upon completion, the reaction mixture was diluted by addition of H₂O (20 mL), and then extracted with DCM (10 mL*3). The combined organic layers were washed with brine (20 mL), dried over Na₂SO₄, filtered and concentrated under reduced pressure to give a residue. The residue was purified by column chromatography (SiO₂, Petroleum ether/Ethyl acetate=1011 to 011) to give the product methyl (2S)-2-[[2-[[(2S)-2-(benzyloxycarbonylamino)-3-(1-naphthyl)propanoyl]amino]-3-(4,4-difluorocyclohexyl)propanoyl]amino]-3-[(3S)-2-oxopyrrolidin-3-yl]propanoate (250 mg, 353.72 umol, 76.20% yield) as a yellow solid. MS (ESI) m/z 707.3 [M+H]⁺

Step 3: benzylN-[(1S)-2-[[2-[[(1S)-2-amino-2-oxo-1-[[(3S)-2-oxopyrrolidin-3-yl]methyl]ethyl]amino]-1-[(4,4-difluorocyclohexyl)methyl]-2-oxo-ethyl]amino]-1-(1-naphthylmethyl)-2-oxo-ethyl]carbamate

A mixture of methyl (2S)-2-[[2-[[(2S)-2-(benzyloxycarbonylamino)-3-(1-naphthyl)propanoyl]amino]-3-(4,4-difluorocyclohexyl)propanoyl]amino]-3-[(3S)-2-oxopyrrolidin-3-yl]propanoate (250.00 mg, 353.72 umol, 1 eq) in NH₃/MeOH (7 M, 16.30 mL, 322.66 eq) was stirred at 20° C. for 16 h. Upon completion, the mixture was concentrated under the reduced pressured to give the product benzyl N-[(1S)-2-[[2-[[(1S)-2-amino-2-oxo-1-[[(3S)-2-oxopyrrolidin-3-yl]methyl]ethyl]amino]-1-[(4,4-difluorocyclohexyl)methyl]-2-oxo-ethyl]amino]-1-(1-naphthylmethyl)-2-oxo-ethyl]carbamate (230 mg, crude) as a yellow solid. MS (ESI) m/z 692.3 [M+H]⁺

Step 4: benzylN-[(1S)-2-[[2-[[(1S)-1-cyano-2-[(3S)-2-oxopyrrolidin-3-yl]ethyl]amino]-1-[(4,4-difluorocyclohexyl)methyl]-2-oxo-ethyl]amino]-1-(1-naphthylmethyl)-2-oxo-ethyl]carbamate

To a solution of benzyl N-[(1S)-2-[[2-[[(1S)-2-amino-2-oxo-1-[[(3S)-2-oxopyrrolidin-3-yl]methyl]ethyl]amino]-1-[(4,4-difluorocyclohexyl)methyl]-2-oxo-ethyl]amino]-1-(1-naphthylmethyl)-2-oxo-ethyl]carbamate (220 mg, 318.03 umol, 1 eq) in DCM (5 mL) was added Burgess reagent (227.36 mg, 954.08 umol, 3 eq). The mixture was stirred at 20° C. for 5 h. Upon completion, the mixture was diluted with H₂O (0.5 mL) and then drying with N₂ to give a residue. The residue was purified by column chromatography (SiO₂, Petroleum ether/Ethyl acetate=5/1 to 0/1) to give the product benzyl N-[(1S)-2-[[2 [[(1S)-1-cyano-2-[(3S)-2-oxopyrrolidin-3-yl]ethyl]amino]-1-[(4,4-difluorocyclohexyl)methyl]-2-oxo-ethyl]amino]-1-(1-naphthylmethyl)-2-oxo-ethyl]carbamate (150 mg, 222.64 umol, 70.00% yield) as a white solid. MS (ESI) m/z 674.4 [M+H]⁺

Step 5: benzylN-[(1S)-2-[[2-[[(1S)-1-cyano-2-[(3S)-2-oxopyrrolidin-3-yl]ethyl]amino]-1-[(4,4-dif luorocyclohexyl)methyl]-2-oxo-ethyl]amino]-1-(1-naphthylmethyl)-2-oxo-ethyl]carbamate

BenzylN-[(1S)-2-[[2-[[(1S)-1-cyano-2-[(3S)-2-oxopyrrolidin-3-yl]ethyl]amino]-1-[(4,4-difluorocyclohexyl)methyl]-2-oxo-ethyl]amino]-1-(1-naphthylmethyl)-2-oxo-ethyl]carbamate (150 mg, 222.64 umol, 1 eq) was separated by SFC (column: DAICEL CHIRALPAK IC(250 mm*30 mm, 10 um); mobile phase: [0.1% NH₃H₂O IPA]; B %: 55%-55%, 10 min) to give benzyl N-[(1S)-2-[[2-[[(1S)-1-cyano-2-[(3S)-2-oxopyrrolidin-3-yl]ethyl]amino]-1-[(4,4-difluorocyclohexyl)methyl]-2-oxo-ethyl]amino]-1-(1-naphthylmethyl)-2-oxo-ethyl]carbamate (40 mg, 58.42 umol, 26.24% yield, 98.4% purity) as a white solid. MS (ESI) m/z 674.3 [M+H]⁺

¹H NMR (400 MHz, MeOD-d₄) δ=8.18 (d, J=8.2 Hz, 1H), 7.87 (d, J=7.8 Hz, 1H), 7.81-7.74 (m, 1H), 7.58-7.44 (m, 2H), 7.41-7.18 (m, 7H), 5.03-4.95 (m, 3H), 4.61 4.53 (m, 2H), 4.48-4.36 (m, 1H), 3.57-3.51 (m, 1H), 3.28-3.25 (m, 1H), 2.54-2.51 (m, 1H), 2.35-2.21 (m, 2H), 2.04-1.56 (m, 11H), 1.51-1.35 (m, 1H), 1.33-1.16 (m, 2H)

Benzyl N-[(1S)-2-[[2-[[(1S)-1-cyano-2-[(3S)-2-oxopyrrolidin-3-yl]ethyl]amino]-1-[(4,4-difluorocyclohexyl)methyl]-2-oxo-ethyl]amino]-1-(1-naphthylmethyl)-2-oxo-ethyl]carbamate (45 mg, 66.79 umol, 30.00% yield, 100% purity) was obtained as a white solid. MS (ESI) m/z 674.3 [M+H]⁺

¹H NMR (400 MHz, MeOD-d₄) δ=8.14 (d, J=8.6 Hz, 1H), 7.88 (d, J=8.8 Hz, 1H), 7.79 (d, J=7.2 Hz, 1H), 7.61-7.47 (m, 2H), 7.45-7.25 (m, 7H), 5.25-5.11 (m, 2H), 5.05-4.96 (m, 1H), 4.59-4.43 (m, 1H), 4.18-4.09 (m, 1H), 3.62-3.40 (m, 2H), 3.28-3.27 (m, 1H), 2.55-2.18 (m, 3H), 1.96-1.67 (m, 4H), 1.59-1.14 (m, 7H), 1.11-0.91 (m, 2H), 0.59 (s, 1H)

Example 21: Synthesis of (1R,2S,5S)—N-[(1S)-1-cyan-2-[(6R)-5-oxo-4-azaspiro[2.4]heptan-6-yl]ethyl]-3-[(2S)-3,3-dimethyl-2-(2,2,2-trifluoroethylamino)butanoyl]-6,6-dimethyl-3-azabicyclo[3.1.0]hexane-2-carboxamide (Compound 203)

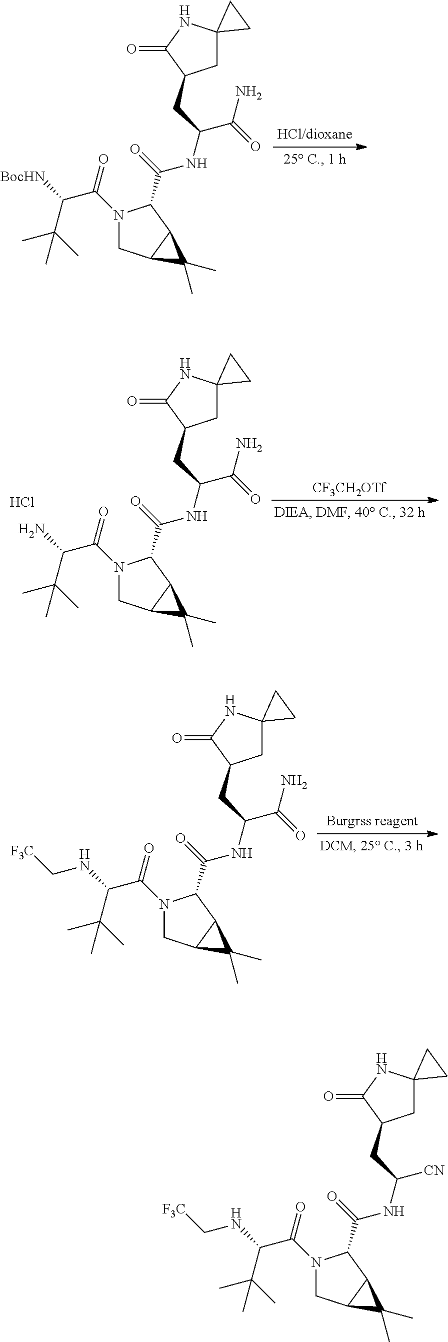 [SMILES: CC(C)(C)OC(=O)N[C@H](C(=O)N1C[C@H]2[C@@H]([C@H]1C(=O)N[C@@H](C[C@@H]1CC3(CC3)NC1=O)C(N)=O)C2(C)C)C(C)(C)C ; Cl ; CC(C)(C)[C@H](N)C(=O)N1C[C@H]2[C@@H]([C@H]1C(=O)N[C@@H](C[C@@H]1CC3(CC3)NC1=O)C(N)=O)C2(C)C ; CC(C)(C)[C@H](NCC(F)(F)F)C(=O)N1C[C@H]2[C@@H]([C@H]1C(=O)N[C@@H](C[C@@H]1CC3(CC3)NC1=O)C(N)=O)C2(C)C ; "CF3CH2OTf" ; CC(C)(C)[C@H](NCC(F)(F)F)C(=O)N1C[C@H]2[C@@H]([C@H]1C(=O)N[C@H](C#N)C[C@@H]1CC3(CC3)NC1=O)C2(C)C]

Step 1: (1R,2S,5S)-3-[(2S)-2-amino-3,3-dimethyl-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide

A solution of tert-butyl N-[(1S)-1-[(1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl-propyl]carbamate (1 g, 1.83 mmol, 1 eq) in HCl/dioxane (4 M, 30 mL, 65.72 eq) was stirred at 25° C. for 1 h. Upon completion, the reaction mixture was concentrated under reduced pressure to give (1R,2S,5S)-3-[(2S)-2-amino-3,3-dimethyl-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide (900 mg, crude, HCl) as a yellow solid. MS (ESI) m/z 448.3 [M+H]⁺.

Step 2: (1R,2S,5S)—N-[(1S)-2-amino-2-oxo-1-[[(6R)-5-oxo-4-azaspiro[2.4]heptan-6-yl]methyl]ethyl]-3-[(2S)-3,3-dimethyl-2-(2,2,2-trifluoroethylamino)butanoyl]-6,6-dimethyl-3-azabicyclo[3.1.0]hexane-2-carboxamide

To a solution of (1R,2S,5S)-3-[(2S)-2-amino-3,3-dimethyl-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide (450 mg, 929.69 umol, 1 eq, HCl) in DMF (10 mL) was added drop-wise DIEA (360.47 mg, 2.79 mmol, 485.81 uL, 3 eq) and 2,2,2-trifluoroethyl trifluoromethanesulfonate (388.41 mg, 1.67 mmol, 1.8 eq). The resulting mixture was stirred at 40° C. for 32 h, and then cooled to room temperature. The resulting mixture was diluted with H₂O 100 mL and extracted with EA (80 mL*3). The combined organic layers were washed with brine 150 mL, dried over Na₂SO₄, concentrated in vacuum. The residue was purified by chromatography (SiO₂, ethyl acetate:petroleum ether=4:1 to 0:1) to afford (1R,2S,5S)—N-[(1S)-2-amino-2-oxo-1-[[(6R)-5-oxo-4-azaspiro[2.4]heptan-6-yl]methyl]ethyl]-3-[(2S)-3,3-dimethyl-2-(2,2,2-trifluoroethylamino)butanoyl]-6,6-dimethyl-3-azabicyclo[3.1.0]hexane-2-carboxamide (280 mg, 528.71 umol, 28.43% yield) as a white solid. MS (ESI) m/z 530.3 [M+H]⁺.

Step 3: (1R,2S,5S)—N-[(1S)-1-cyano-2-[(6R)-5-oxo-4-azaspiro[2.4]heptan-6-yl]ethyl]-3-[(2S)-3,3-dimethyl-2-(2,2,2-trifluoroethylamino)butanoyl]-6,6-dimethyl-3-azabicyclo[3.1.0]hexane-2-carboxamide

To a solution of (1R,2S,5S)—N-[(1S)-2-amino-2-oxo-1-[[(6R)-5-oxo-4-azaspiro[2.4]heptan-6-yl]methyl]ethyl]-3-[(2S)-3,3-dimethyl-2-(2,2,2-trifluoroethylamino)butanoyl]-6,6-dimethyl-3-azabicyclo[3.1.0]hexane-2-carboxamide (250 mg, 472.06 umol, 1 eq) in DCM (10 mL) was added Burgess reagent (449.99 mg, 1.89 mmol, 4 eq), the mixture was stirred at 25° C. for 3 h. Upon completion, water (1.5 mL) was added to the solution, then dried by blowing N₂. The residue was purified by prep-HPLC (column: Waters Xbridge Prep OBD C18 150*40 mm*10 um; mobile phase: [water(NH₃H₂O+NH₄HCO₃)-ACN]; B %: 30%-60%, 8 min) to give (1R,2S,5S)—N-[(1S)-1-cyano-2-[(6R)-5-oxo-4-azaspiro[2.4]heptan-6-yl]ethyl]-3-[(2S)-3,3-dimethyl-2-(2,2,2-trifluoroethylamino)butanoyl]-6,6-dimethyl-3-azabicyclo[3.1.0]hexane-2-carboxamide (95 mg, 180.13 umol, 38.16% yield, 97% purity) was obtained as a white solid. MS (ESI) m/z 512.2 [M+H]⁺.

¹H NMR (400 MHz, MeOD-d₄) δ=9.06 (d, J=8.4 Hz, 1H), 7.73 (s, 1H), 5.01 4.82 (m, 1H), 4.38-4.19 (m, 1H), 3.85-3.76 (m, 1H), 3.66 (d, J=10.6 Hz, 1H), 3.24-3.16 (m, 1H), 3.14 (d, J=11.5 Hz, 1H), 2.97-2.86 (m, 1H), 2.70-2.61 (m, 1H), 2.39-2.32 (m, 1H), 2.25-2.15 (m, 1H), 2.01-1.88 (m, 2H), 1.85-1.74 (m, 1H), 1.59-1.52 (m, 1H), 1.30 (d, J=7.7 Hz, 1H), 1.04 (s, 3H), 0.91 (s, 12H), 0.76-0.69 (m, 1H), 0.64-0.44 (m, 3H).

Example 22: Synthesis of benzyl N-[(1S)-1-[(1R,2S,5S)-2-[[(1S)-1-cyano-2-[(3S)-2-oxopyrrolidin-3-yl]ethyl]carbamoyl]-6,6-dimethyl-3-azabicyclo[3.1.0]hexane-3-carbonyl]-2,2-dimethyl-propyl]carbamate

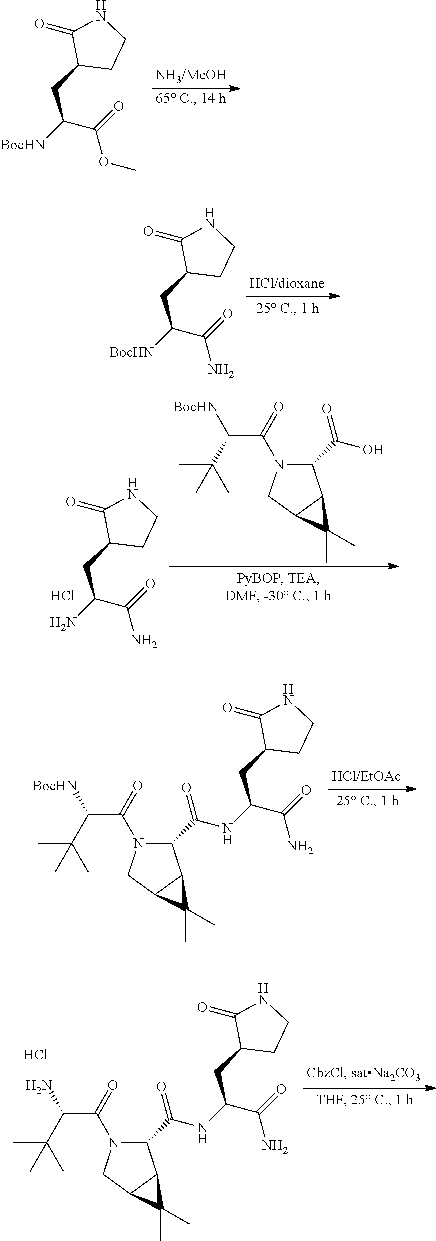

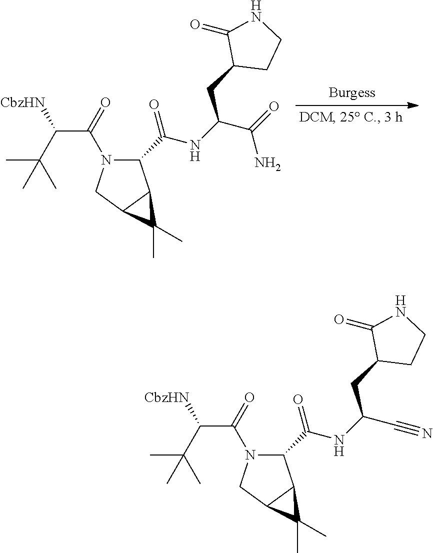 [SMILES: CC(C)(C)[C@H](NC(=O)OCc1ccccc1)C(=O)N1C[C@H]2[C@@H]([C@H]1C(=O)N[C@@H](C[C@@H]1CCNC1=O)C(N)=O)C2(C)C ; CC(C)(C)[C@H](NC(=O)OCc1ccccc1)C(=O)N1C[C@H]2[C@@H]([C@H]1C(=O)N[C@H](C#N)C[C@@H]1CCNC1=O)C2(C)C]

Step 1: tert-butyl N-[(1S)-2-amino-2-oxo-1-[[(3S)-2-oxopyrrolidin-3-yl]methyl]ethyl]carbamate

A solution of methyl (2S)-2-(tert-butoxycarbonylamino)-3-[(3S)-2-oxopyrrolidin-3-yl]propanoate (2 g, 6.99 mmol, 1 eq) in NH₃/MeOH (7 M, 20 mL, 20.04 eq) was stirred at 65° C. for 14 h. Upon completion, the reaction mixture was concentrated under reduced pressure to dryness to give tert-butyl N-[(1S)-2-amino-2-oxo-1-[[(3S)-2-oxopyrrolidin-3-yl]methyl]ethyl]carbamate (2.0 g, crude) as yellow solid. MS (ESI) m/z 272.0 [M+H]⁺.

Step 2: (2S)-2-amino-3-[(3S)-2-oxopyrrolidin-3-yl]propanamide

A mixture of tert-butyl N-[(1S)-2-amino-2-oxo-1-[[(3S)-2-oxopyrrolidin-3-yl]methyl]ethyl]carbamate (500 mg, 1.75 mmol, 95% purity, 1 eq) in HCl/dioxane (4 M, 5 mL, 11.42 eq) was stirred at 25° C. for 1 h. The mixture was concentrated under reduced pressure to dryness to give (2S)-2-amino-3-[(3S)-2-oxopyrrolidin-3-yl]propanamide (380 mg, crude, HCl) as white solid. MS (ESI) m/z 172.2 [M+H]⁺.

Step 3: tert-butyl N-[(1S)-1-[(1R,2S,5S)-2-[[(1S)-2-amino-2-oxo-1-[[(3S)-2-oxopyrrolidin-3-yl]methyl]ethyl]carbamoyl]-6,6-dimethyl-3-azabicyclo[3.1.0]hexane-3-carbonyl]-2,2-dimethyl-propyl]carbamate

To a mixture of (1R,2S,5S)-3-[(2S)-2-(tert-butoxycarbonylamino)-3,3-dimethyl-butanoyl]-6,6-dimethyl-3-azabicyclo[3.1.0]hexane-2-carboxylic acid (612.97 mg, 1.58 mmol, 95% purity, 1 eq) and (2S)-2-amino-3-[(3S)-2-oxopyrrolidin-3-yl]propanamide (380 mg, 1.74 mmol, 95% purity, 1.1 eq, HCl) in DMF (4 mL) was added PyBOP (822.42 mg, 1.58 mmol, 1 eq), then a solution of TEA (319.84 mg, 3.16 mmol, 439.95 uL, 2 eq) in DMF (2 mL) was added and stirred at −30° C. for 1 h. Upon completion, the reaction mixture was quenched by water (20 mL), and then extracted with DCM (10 mL*2). The combined organic layers were washed with brine (10 mL), dried over Na₂SO₄, filtered and concentrated under reduced pressure to give a residue. The residue was purified by prep-TLC (SiO₂, DCM:MeOH=10:1) to give tert-butyl N-[(1S)-1-[(1R,2S,5S)-2-[[(1S)-2-amino-2-oxo-1-[[(3S)-2-oxopyrrolidin-3-yl]methyl]ethyl]carbamoyl]-6,6-dimethyl-3-azabicyclo[3.1.0]hexane-3-carbonyl]-2,2-dimethyl-propyl]carbamate (750 mg, 1.22 mmol, 77.33% yield, 85% purity) as yellow solid. MS (ESI) m/z 522.3 [M+H]⁺.

Step 4: (1R,2S,5S)-3-[(2S)-2-amino-3,3-dimethyl-butanoyl]-N-[(1S)-2-amino-2-oxo-1-[[(3S)-2-oxopyrrolidin-3-yl]methyl]ethyl]-6,6-dimethyl-3-azabicyclo[3.1.0]hexane-2-carboxamide

A mixture of tert-butyl N-[(1S)-1-[(1R,2S,5S)-2-[[(1S)-2-amino-2-oxo-1-[[(3S)-2-oxopyrrolidin-3-yl]methyl]ethyl]carbamoyl]-6,6-dimethyl-3-azabicyclo[3.1.0]hexane-3-carbonyl]-2,2-dimethyl-propyl]carbamate (700 mg, 1.14 mmol, 85% purity, 1 eq) in HCl/EtOAc (4 M, 7 mL, 24.55 eq) was stirred at 25° C. for 1 h. The mixture was concentrated under reduced pressure to dryness to give (1R,2S,5S)-3-[(2S)-2-amino-3,3-dimethyl-butanoyl]-N-[(1S)-2-amino-2-oxo-1-[[(3S)-2-oxopyrrolidin-3-yl]methyl]ethyl]-6,6-dimethyl-3-azabicyclo[3.1.0]hexane-2-carboxamide (650 mg, crude, HCl) as white solid. MS (ESI) m/z 422.1 [M+H]⁺.

Step 5: benzyl N-[(1S)-1-[(1R,2S,5S)-2-[[(1S)-2-amino-2-oxo-1-[[(3S)-2-oxopyrrolidin-3-yl]methyl]ethyl]carbamoyl]-6,6-dimethyl-3-azabicyclo[3.1.0]hexane-3-carbonyl]-2,2-dimethyl-propyl]carbamate

To a solution of (1R,2S,5S)-3-[(2S)-2-amino-3,3-dimethyl-butanoyl]-N-[(1S)-2-amino-2-oxo-1-[[(3S)-2-oxopyrrolidin-3-yl]methyl]ethyl]-6,6-dimethyl-3-azabicyclo[3.1.0]hexane-2-carboxamide (600 mg, 982.55 umol, 75% purity, 1 eq, HCl) in THE (6 mL) was added sat.Na₂CO₃ (247.85 mg, 2.34 mmol, 0.5 mL, 2.38 eq) (adjust pH-8). Then, CbzCl (335.23 mg, 1.97 mmol, 279.36 uL, 2 eq) was added slowly, and the mixture was stirred at 25° C. for 1 h under N₂. The mixture was concentrated in vacuum and added H₂O (800 mL), extracted with ethyl acetate (400 mL*3), the organic layer was washed with brine, dried over anhydrous Na₂SO₄, filtered and concentrated under reduced pressure to give a residue. The crude product was purified by trituration (PE:EA=2:1) to give benzyl N [(1S)-1-[(1R,2S,5S)-2-[[(1S)-2-amino-2-oxo-1-[[(3S)-2-oxopyrrolidin-3-yl]methyl]ethyl]carbamoyl]-6,6-dimethyl-3-azabicyclo[3.1.0]hexane-3-carbonyl]-2,2-dimethyl-propyl]carbamate (490 mg, crude) as yellow solid. MS (ESI) m/z 556.3 [M+H]⁺.

Step 6: benzyl N-[(1S)-1-[(1R,2S,5S)-2-[[(1S)-1-cyano-2-[(3S)-2-oxopyrrolidin-3-yl]ethyl]carbamoyl]-6,6-dimethyl-3-azabicyclo[3.1.0]hexane-3-carbonyl]-2,2-dimethyl-propyl]carbamate

A mixture of benzyl N-[(1S)-1-[(1R,2S,5S)-2-[[(1S)-2-amino-2-oxo-1-[[(3S)-2-oxopyrrolidin-3-yl]methyl]ethyl]carbamoyl]-6,6-dimethyl-3-azabicyclo[3.1.0]hexane-3-carbonyl]-2,2-dimethyl-propyl]carbamate (470 mg, 592.08 umol, 70% purity, 1 eq) in DCM (5 mL) was added Burgess reagent (423.30 mg, 1.78 mmol, 3 eq) and stirred at 25° C. for 3 h. Upon completion, the mixture was quenched with water (0.5 mL) and concentrated under reduced pressure (<30° C.) to give a residue. The residue was purified by prep-HPLC(column: Waters Xbridge BEH C18 250*50 mm*10 um; mobile phase: [water(NH₄HCO₃)-ACN]; B %: 30%-60%, 10 min) to give benzyl N-[(1S)-1-[(1R,2S,5S)-2-[[(1S)-1-cyano-2-[(3S)-2-oxopyrrolidin-3-yl]ethyl]carbamoyl]-6,6-dimethyl-3-azabicyclo[3.1.0]hexane-3-carbonyl]-2,2-dimethyl-propyl]carbamate (120 mg, 223.19 umol, 37.70% yield, 100% purity) as white solid. MS (ESI) m/z 538.2 [M+H]⁺.

¹H NMR (400 MHz, DMSO-d₆) δ=8.98 (d, J=8.6 Hz, 1H), 7.65 (s, 1H), 7.43 7.25 (m, 6H), 5.12-4.89 (m, 3H), 4.18-4.02 (m, 2H), 3.91-3.75 (m, 2H), 3.18-3.09 (m, 1H), 3.07-2.98 (m, 1H), 2.45-2.35 (m, 1H), 2.21-2.02 (m, 2H), 1.77-1.62 (m, 2H), 1.57 1.50 (m, 1H), 1.28 (d, J=7.7 Hz, 1H), 1.02 (s, 3H), 0.93 (s, 9H), 0.85 (s, 3H)

Example 23: Synthesis of (1R⁵S)-3-[(2S,3R)-3-tert-butoxy-2-[(2,2,2-trifluoroacetyl)amino]butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (Compound 263)

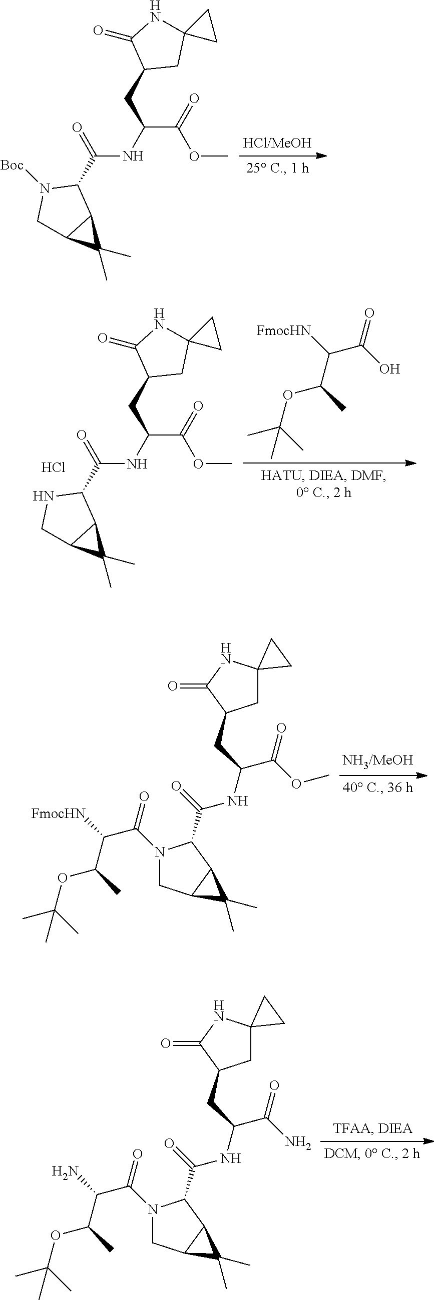

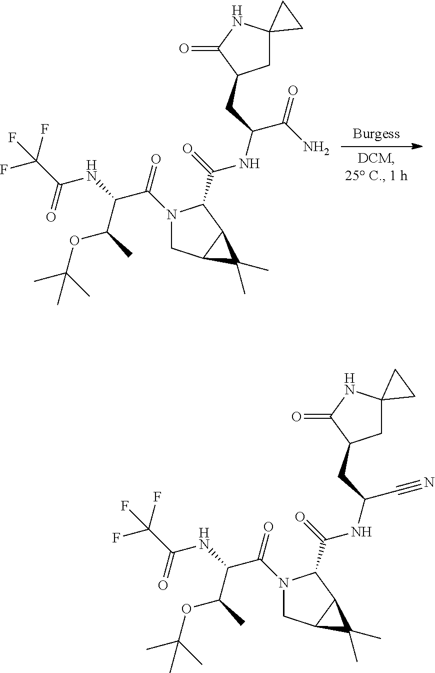 [SMILES: C[C@@H](OC(C)(C)C)[C@H](NC(=O)C(F)(F)F)C(=O)N1C[C@H]2[C@@H]([C@H]1C(=O)N[C@@H](C[C@@H]1CC3(CC3)NC1=O)C(N)=O)C2(C)C ; C[C@@H](OC(C)(C)C)[C@H](NC(=O)C(F)(F)F)C(=O)N1C[C@H]2[C@@H]([C@H]1C(=O)N[C@H](C#N)C[C@@H]1CC3(CC3)NC1=O)C2(C)C]

Step 1: methyl (2S)-2-[[(1R,2S,5S)-6,6-dimethyl-3-azabicyclo[3.1.0]hexane-2-carbonyl]amino]-3-[(6R)-5-oxo-4-azaspiro[2.4]heptan-6-yl]propanoate

A mixture of tert-butyl (1R,2S,5S)-2-[[(1S)-2-methoxy-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carboxylate (0.25 g, 556.13 umol, 1 eq) in HCl/MeOH (4 M, 8 mL, 57.54 eq) was stirred at 25° C. for 1 h. Upon completion, the reaction mixture was concentrated under reduced pressure to give methyl (2S)-2-[[(1R,2S,5S)-6,6-dimethyl-3-azabicyclo[3.1.0]hexane-2-carbonyl]amino]-3-[(6R)-5-oxo-4-azaspiro[2.4]heptan-6-yl]propanoate (200 mg, crude, HCl) as a white solid.

Step 2: methyl (2S)-2-[[(1R,2S,5S)-3-[(2S,3R)-3-tert-butoxy-2-(9H fluoren-9-ylmethoxycarbonylamino)butanoyl]-6,6-dimethyl-3-azabicyclo[3.1.0]hexane-2-carbonyl]amino]-3-[(6R)-5-oxo-4-azaspiro[2.4]heptan-6-yl]propanoate

To a solution of methyl (2S)-2-[[(1R,2S,5S)-6,6-dimethyl-3-azabicyclo[3.1.0]hexane-2-carbonyl]amino]-3-[(6R)-5-oxo-4-azaspiro[2.4]heptan-6-yl]propanoate (200 mg, 518.29 umol, 1 eq, HCl) and (2S,3R)-3-tert-butoxy-2-(9H-fluoren-9-ylmethoxycarbonylamino)butanoic acid (185.40 mg, 466.46 umol, 0.9 eq) in DMF (12 mL) was added HATU (394.14 mg, 1.04 mmol, 2 eq), then DIEA (200.95 mg, 1.55 mmol, 270.82 uL, 3 eq) was added. The mixture was stirred at 0° C. for 2 h. Upon completion, the reaction mixture was diluted with water (15 mL) and extracted with DCM (5 mL*3). The combined organic layers were dried over Na₂SO₄, filtered and concentrated under reduced pressure to give a residue. The residue was purified by prep-TLC (SiO₂, petroleum ether:Ethyl acetate=1:2) to afford methyl (2S)-2-[[(1R,2S,5S)-3-[(2S,3R)-3-tert-butoxy-2-(9H-fluoren-9-ylmethoxycarbonylamino)butanoyl]-6,6-dimethyl-3-azabicyclo[3.1.0]hexane-2-carbonyl]amino]-3-[(6R)-5-oxo-4-azaspiro[2.4]heptan-6-yl]propanoate (238 mg, 326.53 umol, 63.00% yield) as a white solid.

Step 3: (1R,2S,5S)-3-[(2S,3R)-2-amino-3-tert-butoxy-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide

A solution of methyl (2S)-2-[[(1R,2S,5S)-3-[(2S,3R)-3-tert-butoxy-2-(9H-fluoren-9-ylmethoxycarbonylamino)butanoyl]-6,6-dimethyl-3-azabicyclo[3.1.0]hexane-2-carbonyl]amino]-3-[(6R)-5-oxo-4-azaspiro[2.4]heptan-6-yl]propanoate (230 mg, 315.56 umol, 1 eq) in NH₃/MeOH (7 M, 9.99 mL, 221.65 eq) was stirred at 40° C. for 36 h. Upon completion, the reaction mixture was concentrated under reduced pressure to give (1R,2S,5S)-3-[(2S,3R)-2-amino-3-tert-butoxy-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide (0.1 g, 193.24 umol, 61.24% yield, 95% purity) as a white solid.

Step 4: (1R,2S,5S)—N-[(1S)-2-amino-2-oxo-1-[[(6R)-5-oxo-4-azaspiro[2.4]heptan-6-yl]methyl]ethyl]-3-[(2S,3R)-3-tert-butoxy-2-[(2,2,2-trifluoroacetyl)amino]butanoyl]-6,6-dimethyl-3-azabicyclo[3.1.0]hexane-2-carboxamide

A mixture of (1R,2S,5S)-3-[(2S,3R)-2-amino-3-tert-butoxy-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide (100 mg, 203.41 umol, 1 eq) in DCM (2 mL) was added DIPEA (78.87 mg, 610.23 umol, 106.29 uL, 3 eq), and then TFAA (85.44 mg, 406.82 umol, 56.59 uL, 2 eq) in DCM (0.1 mL) was drop-wise for 0.1 h. The resulting mixture was stirred at 0° C. for 1.9 h. Upon completion, the reaction mixture was poured into saturated NaHCO₃(8 mL) and extracted with DCM (4 mL*3). The combined organic layers was filtered and concentrated under reduced pressure to give (1R,2S,5S)—N-[(1S)-2-amino-2-oxo-1-[[(6R)-5-oxo-4-azaspiro[2.4]heptan-6-yl]methyl]ethyl]-3-[(2S,3R)-3-tert-butoxy-2-[(2,2,2-trifluoroacetyl)amino]butanoyl]-6,6-dimethyl-3-azabicyclo[3.1.0]hexane-2-carboxamide (120 mg, crude) as a white solid.

Step 5: (1R,2S,5S)-3-[(2S,3R)-3-tert-butoxy-2-[(2,2,2-trifluoroacetyl)amino]butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide

To a solution of (1R,2S,5S)—N-[(1S)-2-amino-2-oxo-1-[[(6R)-5-oxo-4-azaspiro[2.4]heptan-6-yl]methyl]ethyl]-3-[(2S,3R)-3-tert-butoxy-2-[(2,2,2-trifluoroacetyl)amino]butanoyl]-6,6-dimethyl-3-azabicyclo[3.1.0]hexane-2-carboxamide (120 mg, 204.21 umol, 1 eq) in DCM (1 mL) was added Burgess reagent (48.67 mg, 204.21 umol, 1 eq), the mixture was stirred at 25° C. for 1 h. Upon completion, water (1 mL) was added to the solution, then dried by blowing N₂. The crude product was purified by prep-HPLC (column: Phenomenex C18 80*40 mm*3 um; mobile phase: [water(NH₄HCO₃)-ACN]; B %: 25%-55%, 8 min) to give (1R,2S,5S)-3-[(2S,3R)-3-tert-butoxy-2-[(2,2,2-trifluoroacetyl)amino]butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (0.02 g, 34.65 umol, 16.97% yield, 98.7% purity) as a white solid. MS (ESI) m/z 569.3 [M+H]⁺.

¹H NMR (400 MHz, DMSO-d₆) δ=9.79-9.63 (m, 1H), 8.99 (d, J=8.4 Hz, 1H), 7.78 (s, 1H), 4.94 (ddd, J=5.4, 8.3, 10.4 Hz, 1H), 4.40 (br t, J=7.1 Hz, 1H), 4.13 (s, 1H), 3.97 (dd, J=5.3, 10.2 Hz, 1H), 3.90 (dd, J=6.2, 8.1 Hz, 1H), 3.68-3.63 (m, 1H), 2.67 2.59 (m, 1H), 2.26-2.10 (m, 1H), 2.04-1.89 (m, 2H), 1.83 (ddd, J=5.6, 10.5, 13.7 Hz, 1H), 1.57 (dd, J=5.6, 7.3 Hz, 1H), 1.33 (d, J=7.6 Hz, 1H), 1.17-1.03 (m, 15H), 0.84 (s, 3H), 0.80-0.72 (m, 1H), 0.64-0.51 (m, 3H).

Example 24: Synthesis of (1R,2S,5S)-3-[(2S,3S)-3-tert-butoxy-2-[(2,2,2-trifluoroacetyl)amino]butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (Compound 264)

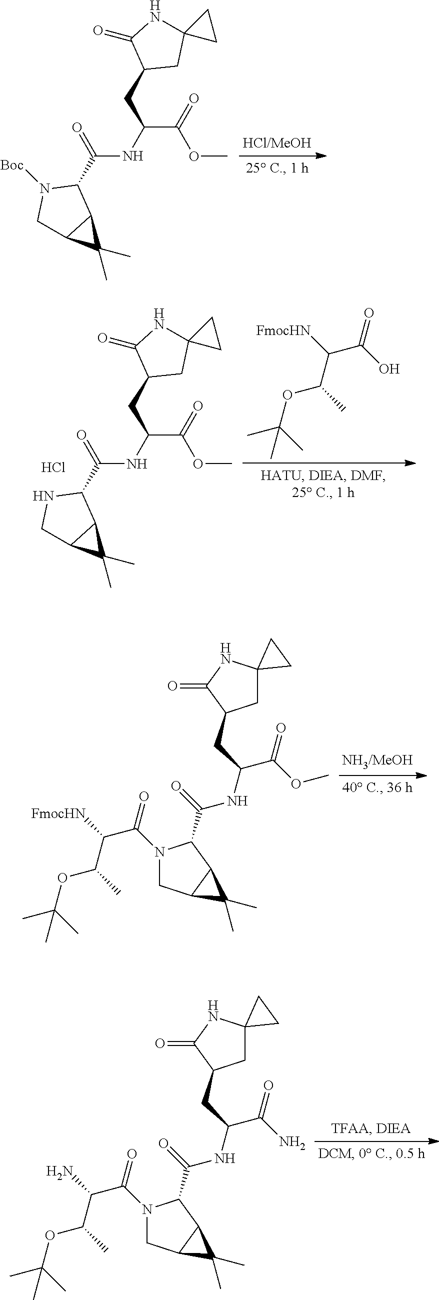

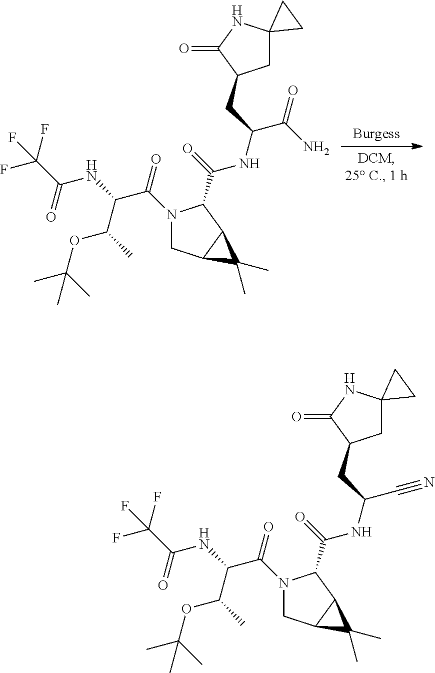 [SMILES: C[C@H](OC(C)(C)C)[C@H](NC(=O)C(F)(F)F)C(=O)N1C[C@H]2[C@@H]([C@H]1C(=O)N[C@@H](C[C@@H]1CC3(CC3)NC1=O)C(N)=O)C2(C)C ; C[C@H](OC(C)(C)C)[C@H](NC(=O)C(F)(F)F)C(=O)N1C[C@H]2[C@@H]([C@H]1C(=O)N[C@H](C#N)C[C@@H]1CC3(CC3)NC1=O)C2(C)C]

Step 1: methyl (2S)-2-[[(1R,2S,5S)-6,6-dimethyl-3-azabicyclo[3.1.0]hexane-2-carbonyl]amino]-3-[(6R)-5-oxo-4-azaspiro[2.4]heptan-6-yl]propanoate

To a mixture of tert-butyl (1R,2S,5S)-2-[[(1S)-2-methoxy-2-oxo-l-[[(6R)-5-oxo-4-azaspiro[2.4]heptan-6-yl]methyl]ethyl]carbamoyl]-6,6-dimethyl-3-azabicyclo[3.1.0]hexane-3-carboxylate (300 mg, 667.35 umol, 1 eq) was added HCl/MeOH (4 M, 12.00 mL, 71.93 eq). The mixture was stirred at 25° C. for 1 h. Upon completion, The reaction mixture was concentrated under reduced pressure to give methyl (2S)-2-[(1R,2S,5S)-6,6-dimethyl-3-azabicyclo[3.1.0]hexane-2-carbonyl]amino]-3-[(6R)-5-oxo-4-azaspiro[2.4]heptan-6-yl]propanoate (250 mg, 647.86 umol, 97.08% yield, HCl) was obtained as a colourless oil.

Step 2: methyl (2S)-2-[[(1R,2S,5S)-3-[(2S,3S)-3-tert-butoxy-2-(9H fluoren-9-ylmethoxycarbonylamino)butanoyl]-6,6-dimethyl-3-azabicyclo[3.1.0]hexane-2-carbonyl]amino]-3-[(6R)-5-oxo-4-azaspiro[2.4]heptan-6-yl]propanoate

To a mixture of (2S,3S)-3-tert-butoxy-2-(9H-fluoren-9-ylmethoxycarbonylamino)butanoic acid (194.67 mg, 489.78 umol, 0.9 eq) in DMF (15 mL) was added DIEA (211.00 mg, 1.63 mmol, 284.37 uL, 3 eq), then methyl (2S)-2-[[(1R,2S,5S)-6,6-dimethyl-3-azabicyclo[3.1.0]hexane-2-carbonyl]amino]-3-[(6R)-5-oxo-4-azaspiro[2.4]heptan-6-yl]propanoate (210 mg, 544.20 umol, 1 eq, HCl) and HATU (413.85 mg, 1.09 mmol, 2 eq) was added. The mixture was stirred at 25° C. for 1 h. Upon completion, the reaction mixture was diluted with H₂O 20 mL and extracted with EA 40 mL (20 mL*2). The combined organic layers were washed with brine 20 mL (20 mL*1), dried over Na₂SO₄, filtered and concentrated under reduced pressure to give a residue. The residue was purified by column chromatography (SiO₂, Petroleum ether:Ethyl acetate=5:1 to 0:1) to afford (2S) 2-[[(1R,2S,5S)-3-[(2S,3S)-3-tert-butoxy-2-(9H-fluoren-9-ylmethoxycarbonylamino)butanoyl]-6,6-dimethyl-3-azabicyclo[3.1.0]hexane-2-carbonyl]amino]-3-[(6R)-5-oxo-4-azaspiro[2.4]heptan-6-yl]propanoate (340 mg, 466.47 umol, 85.72% yield) as a colourless oil. MS (ESI) m/z 729.4 [M+H]⁺.

Step 3: (1R,2S,5S)-3-[(2S,3S)-2-amino-3-tert-butoxy-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide

A solution of methyl (2S)-2-[[(1R,2S,5S)-3-[(2S,3S)-3-tert-butoxy-2-(9H-fluoren-9-ylmethoxycarbonylamino)butanoyl]-6,6-dimethyl-3-azabicyclo[3.1.0]hexane-2-carbonyl]amino]-3-[(6R)-5-oxo-4-azaspiro[2.4]heptan-6-yl]propanoate (340 mg, 466.47 umol, 1 eq) in NH₃/MeOH (7 M, 60.00 mL, 900.37 eq) was stirred at 40° C. for 36 h. Upon completion, the reaction mixture was concentrated under reduced pressure to give a residue. The residue was purified by prep-TLC (SiO₂, Petroleum ether:Ethyl acetate=0:1).

Compound (1R,2S,5S)-3-[(2S,3S)-2-amino-3-tert-butoxy-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide (171 mg, 347.83 umol, 74.57% yield) was obtained as a colourless oil. MS (ESI) m/z 492.4 [M+H]⁺.

Step 4: (1R,2S,5S)—N-[(1S)-2-amino-2-oxo-1-[[(6R)-5-oxo-4-azaspiro[2.4]heptan-6-yl]methyl]ethyl]-3-[(2S,3S)-3-tert-butoxy-2-[(2,2,2-trifluoroacetyl)amino]butanoyl]-6,6-dimethyl-3-azabicyclo[3.1.0]hexane-2-carboxamide

To a solution of (1R,2S,5S)-3-[(2S,3S)-2-amino-3-tert-butoxy-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide (160 mg, 325.45 umol, 1 eq) in DCM (8 mL) was added DIEA (126.19 mg, 976.36 umol, 170.06 uL, 3 eq). Then, TFAA (136.71 mg, 650.91 umol, 90.54 uL, 2 eq) in DCM (2 mL) was added dropwise at 0° C. The resulting mixture was stirred at 0° C. for 0.5 h. Upon completion, the reaction mixture was diluted with H₂O 20 mL and extracted with DCM 40 mL (20 mL*2). The combined organic layers were washed with brine 20 mL (20 mL*1), dried over Na₂SO₄, filtered and concentrated under reduced pressure to give (1R,2S,5S)—N-[(1S)-2-amino-2-oxo-1-[[(6R)-5-oxo-4-azaspiro[2.4]heptan-6-yl]methyl]ethyl]-3-[(2S,3S)-3-tert-butoxy-2-[(2,2,2-trifluoroacetyl)amino]butanoyl]-6,6-dimethyl-3-azabicyclo[3.1.0]hexane-2-carboxamide (185 mg, 273.90 umol, 84.16% yield, 87% purity) as a colourless oil.

Step 5: (1R,2S,5S)-3-[(2S,3S)-3-tert-butoxy-2-[(2,2,2-trifluoroacetyl)amino]butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide

To a mixture of (1R,2S,5S)—N-[(1S)-2-amino-2-oxo-1-[[(6R)-5-oxo-4-azaspiro[2.4]heptan-6-yl]methyl]ethyl]-3-[(2S,3S)-3-tert-butoxy-2-[(2,2,2-trifluoroacetyl)amino]butanoyl]-6,6-dimethyl-3-azabicyclo[3.1.0]hexane-2-carboxamide (175 mg, 297.81 umol, 1 eq) in DCM (15 mL) was added Burgess reagent (212.91 mg, 893.42 umol, 3 eq). The mixture was stirred at 25° C. for 1 h. Upon completion, the reaction mixture was quenched by addition H₂O 20 mL at 25° C., and then extracted with DCM 40 mL (20 mL*2). The combined organic layers were washed with brine 20 mL (20 mL*1), dried over Na₂SO₄, filtered and concentrated under reduced pressure to give a residue. The residue was purified by neutral prep-HPLC(column: Phenomenex C18 75*30 mm*3 um; mobile phase: [water(NH₄HCO₃)-ACN]; B %: 35%-55%, 8 min) to give (1R,2S,5S)-3-[(2S,3S)-3-tert-butoxy-2-[(2,2,2-trifluoroacetyl)amino]butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (70 mg, 122.89 umol, 41.27% yield, 100% purity) as a yellow solid. MS (ESI) m/z 570.2 [M+H]⁺.

¹H NMR (400 MHz, DMSO-d₆) δ=9.73 (br d, J=5.1 Hz, 1H), 9.13-9.01 (m, 1H), 7.79-7.75 (m, 1H), 4.90-4.82 (m, 1H), 4.37 (br s, 1H), 4.11 (s, 1H), 4.06-3.99 (m, 1H), 3.93 (dd, J=5.5, 10.6 Hz, 1H), 3.72 (d, J=10.7 Hz, 1H), 2.58 (br dd, J=4.6, 10.1 Hz, 1H), 2.25-2.09 (m, 1H), 2.00-1.91 (m, 2H), 1.83 (ddd, J=5.8, 10.2, 13.6 Hz, 1H), 1.64 1.50 (m, 1H), 1.27 (d, J=7.6 Hz, 1H), 1.13-0.99 (m, 15H), 0.87 (s, 3H), 0.77-0.70 (m, 1H), 0.65-0.50 (m, 3H).

Example 25: Synthesis of (1R,2S,5S)—N—((S)-1-cyan-2-((R)-5-oxo-4-azaspiro[2.4]heptan-6-yl)ethyl)-3-((S)-3-methoxy-3-methyl-2-(2,2,2-trifluoroacetamido)butanoyl)-6,6-dimethyl-3-azabicyclo[3.1.0]hexane-2-carboxamide (Compound 139)

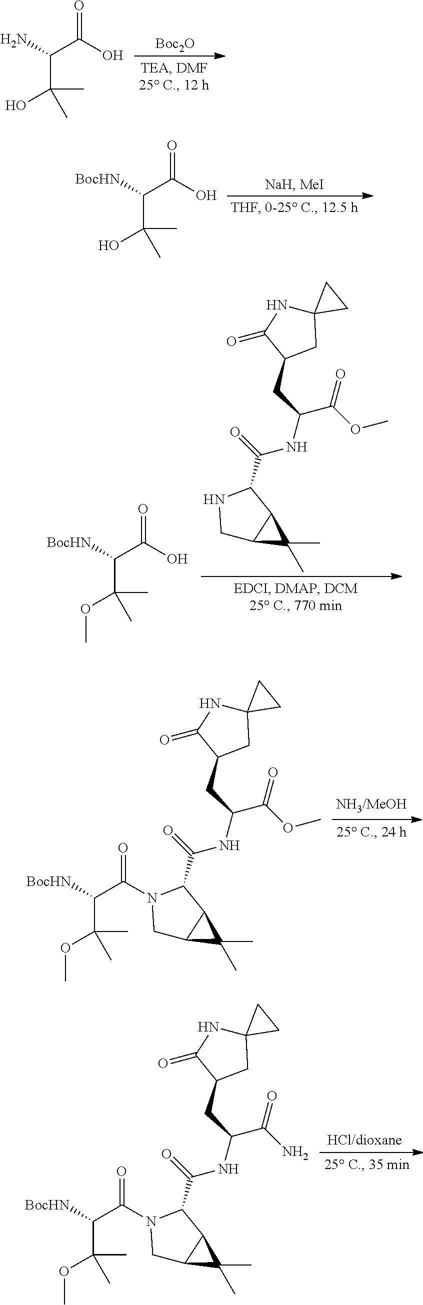

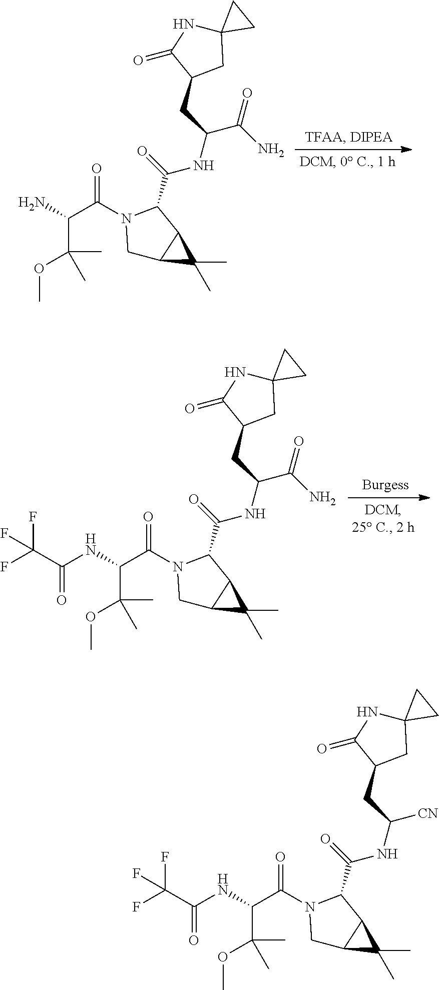 [SMILES: COC(C)(C)[C@H](N)C(=O)N1C[C@H]2[C@@H]([C@H]1C(=O)N[C@@H](C[C@@H]1CC3(CC3)NC1=O)C(N)=O)C2(C)C ; COC(C)(C)[C@H](NC(=O)C(F)(F)F)C(=O)N1C[C@H]2[C@@H]([C@H]1C(=O)N[C@@H](C[C@@H]1CC3(CC3)NC1=O)C(N)=O)C2(C)C ; COC(C)(C)[C@H](NC(=O)C(F)(F)F)C(=O)N1C[C@H]2[C@@H]([C@H]1C(=O)N[C@H](C#N)C[C@@H]1CC3(CC3)NC1=O)C2(C)C]

Step 1: (S)-2-((tert-butoxycarbonyl)amino)-3-hydroxy-3-methylbutanoic acid

To a solution of (S)-2-amino-3-hydroxy-3-methylbutanoic acid (800 mg, 6.01 mmol, 1 eq) in DMF (16 mL) was added tert-butoxycarbonyl tert-butyl carbonate (1.31 g, 6.01 mmol, 1.38 mL, 1 eq), TEA (607.99 mg, 6.01 mmol, 836.30 uL, 1 eq), and then the mixture was stirred at 25° C. for 12 h. Upon completion, the reaction mixture was diluted with water (50 mL), pH adjusted to 10 with 6 N NaOH, washed with EA (50 mL*2), and the aqueous phase was isolated. The aqueous phase was adjusted pH to 4 with 1 N HCl then extracted with DCM (50 mL*3). The organic phase was isolated, dired over Na₂SO₄, filtered and concentrated to give (S)-2-((tert-butoxycarbonyl) amino)-3-hydroxy-3-methylbutanoic acid (1.1 g, crude) as light yellow oil.

Step 2: (S)-2-((tert-butoxycarbonyl)amino)-3-methoxy-3-methylbutanoic acid

To a solution of (S)-2-((tert-butoxycarbonyl)amino)-3-hydroxy-3-methylbutanoic acid (600 mg, 2.57 mmol, 1 eq) in THE (15 mL) was added NaH (514.40 mg, 12.86 mmol, 60% purity, 5 eq) at 0° C. After 30 min, Mel (547.65 mg, 3.86 mmol, 240.20 uL, 1.5 eq) was added at 0° C. and the reaction was stirred for 12 h at 25° C. Upon completion, the reaction mixture was quenched by saturated aqueous ammonium chloride (50 mL), pH adjusted to 4 with 1 N HCl, extracted with EtOAc (50 mL*3). The combined organic layers were washed with brine (50 mL*3), dried over Na₂SO₄, filtered and concentrated under reduced pressure to give (S)-2-((tert-butoxycarbonyl)amino)-3-methoxy-3-methylbutanoic acid (500 mg, crude) as yellow oil.

Step 3: (S)-methyl 2-((1R,2S,5S)-3-((S)-2-((tert-butoxycarbonyl)amino)-3-methoxy-3-methylbutanoyl)-6,6-dimethyl-3-azabicyclo[3.1.0]hexane-2-carboxamido)-3-((R)-5-oxo-4-azaspiro[2.4]heptan-6-yl)propanoate

To a solution of (S)-methyl 2-((1R,2S,5S)-6,6-dimethyl-3-azabicyclo[3.1.0]hexane-2-carboxamido)-3-((R)-5-oxo-4-azaspiro[2.4]heptan-6-yl)propanoate (50 mg, 129.57 umol, 1 eq, HCl) (Batch 7) and (S)-2-((tert-butoxycarbonyl)amino)-3-methoxy-3-methylbutanoic acid (48.06 mg, 194.36 umol, 1.5 eq) in DCM (2 mL), was added DMAP (47.49 mg, 388.72 umol, 3 eq) and EDCI (49.68 mg, 259.14 umol, 2 eq). The mixture was stirred at 25° C. for 50 min. After 12 hours of stirring, the resulting solution was poured into brine (30 mL), and then extracted with EtOAc (30 mL*2), the combined organic phase was washed with 7% citric acid (40 mL*2), NaHCO₃(30 mL) and brine (30 mL*3), dried over Na₂SO₄, filtered and concentrated to give a crude product. The residue was purified by column chromatography (SiO₂, Petroleum ether:Ethyl acetate=5:1 to 0:1) to give (S)-methyl 2-((1R,2S,5S)-3-((S)-2-((tert-butoxycarbonyl)amino)-3-methoxy-3-methylbutanoyl)-6,6-dimethyl-3-azabicyclo[3.1.0]hexane-2-carboxamido)-3-((R)-5-oxo-4-azaspiro[2.4]heptan-6-yl)propanoate (290 mg, 501.13 umol, 55.25% yield) as a white solid.

Step 4: tert-butyl ((S)-1-((1R,2S,5S)-2-(((S)-1-amino-1-oxo-3-((R)-5-oxo-4-azaspiro[2.4]heptan-6-yl)propan-2-yl)carbamoyl)-6,6-dimethyl-3-azabicyclo[3.1.0]hexan-3-yl)-3-methoxy-3-methyl-1-oxobutan-2-yl)carbamate

A solution of (S)-methyl 2-((1R,2S,5S)-3-((S)-2-((tert-butoxycarbonyl)amino)-3-methoxy-3-methylbutanoyl)-6,6-dimethyl-3-azabicyclo[3.1.0]hexane-2-carboxamido)-3-((R)-5-oxo-4-azaspiro[2.4]heptan-6-yl)propanoate (290 mg, 501.13 umol, 1 eq) in NH₃/MeOH (7 M, 8 mL, 111.75 eq) was stirred at 25° C. for 24 h. Upon completion, the reaction mixture was concentrated under reduced pressure to remove the solvent to give tert-butyl ((S)-1 ((1R,2S,5S)-2-(((S)-1-amino-1-oxo-3-((R)-5-oxo-4-azaspiro[2.4]heptan-6-yl)propan-2-yl)carbamoyl)-6,6-dimethyl-3-azabicyclo[3.1.0]hexan-3-yl)-3-methoxy-3-methyl-1-oxobutan-2-yl)carbamate (280 mg, crude) as a yellow solid.

Step 5: (1R,2S,5S)—N—((S)-1-amino-1-oxo-3-((R)-5-oxo-4-azaspiro[2.4]heptan-6-yl)propan-2-yl)-3-((S)-2-amino-3-methoxy-3-methylbutanoyl)-6,6-dimethyl-3-azabicyclo[3.1.0]hexane-2-carboxamide

A solution of tert-butyl ((S)-1-((1R,2S,5S)-2-(((S)-1-amino-1-oxo-3-((R)-5-oxo-4-azaspiro[2.4]heptan-6-yl)propan-2-yl)carbamoyl)-6,6-dimethyl-3-azabicyclo[3.1.0]hexan-3-yl)-3-methoxy-3-methyl-1-oxobutan-2-yl)carbamate (230 mg, 408.03 umol, 1 eq) in HCl/dioxane (4 M, 4.60 mL, 45.09 eq) was stirred at 25° C. for 35 min. Upon completion, the resulting solution was concentrated in vacuum to give (1R,2S,5S)—N—((S)-1-amino-1-oxo-3-((R)-5-oxo-4-azaspiro[2.4]heptan-6-yl)propan-2-yl)-3-((S)-2-amino-3-methoxy-3-methylbutanoyl)-6,6-dimethyl-3-azabicyclo[3.1.0]hexane-2-carboxamide (200 mg, crude, HCl) as a yellow solid.

Step 6: (1R,2S,5S)-N—((S)-1-amino-1-oxo-3-((R)-5-oxo-4-azaspiro[2.4]heptan-6-yl)propan-2-yl)-3-((S)-3-methoxy-3-methyl-2-(2,2,2-trifluoroacetamido)butanoyl)-6,6-dimethyl-3-azabicyclo[3.1.0]hexane-2-carboxamide

To a solution of (1R,2S,5S)-N—((S)-1-amino-1-oxo-3-((R)-5-oxo-4-azaspiro[2.4]heptan-6-yl)propan-2-yl)-3-((S)-2-amino-3-methoxy-3-methylbutanoyl)-6,6-dimethyl-3-azabicyclo[3.1.0]hexane-2-carboxamide (200 mg, 399.98 umol, 1 eq, HCl) in DCM (3 mL) was added DIPEA (155.08 mg, 1.20 mmol, 209.00 uL, 3 eq) at 0° C. Then, TFAA (84.01 mg, 399.98 umol, 55.63 uL, 1 eq) was added dropwise, and the mixture was stirred at 0° C. for 1 h. Upon completion, the resulting solution was poured into H₂O (10 mL), adjusted to pH ˜8 with NaHCO₃ and then extracted with EtOAc (10 mL*2). The combined organic layers were washed with brine (10 mL*3), dried over Na₂SO₄, filtered and concentrated to give (1R,2S,5S)-N—((S)-1-amino-1-oxo-3-((R)-5-oxo-4-azaspiro[2.4]heptan-6-yl)propan-2-yl)-3-((S)-3-methoxy-3-methyl-2-(2,2,2-trifluoroacetamido)butanoyl)-6,6-dimethyl-3-azabicyclo[3.1.0]hexane-2-carboxamide (230 mg, crude) as a white solid.

Step 7: (1R,2S,5S)-N—((S)-1-cyan-2-((R)-5-oxo-4-azaspiro[2.4]heptan-6-yl)ethyl)-3-((S)-3-methoxy-3-methyl-2-(2,2,2-trifluoroacetamido)butanoyl)-6,6-dimethyl-3-azabicyclo[3.1.0]hexane-2-carboxamide

To a solution of (1R,2S,5S)-N—((S)-1-amino-1-oxo-3-((R)-5-oxo-4-azaspiro[2.4]heptan-6-yl)propan-2-yl)-3-((S)-3-methoxy-3-methyl-2-(2,2,2-trifluoroacetamido)butanoyl)-6,6-dimethyl-3-azabicyclo[3.1.0]hexane-2-carboxamide (230 mg, 411.02 umol, 1 eq) in DCM (3 mL) was added Burgess reagent (195.90 mg, 822.05 umol, 2 eq). The resulting mixture was stirred at 25° C. for 1 h. Burgess reagent (97.95 mg, 411.02 umol, 1 eq) was added additionally and the resulting mixture was stirred for another 1 h. Upon completion, the resulting solution was quenched with H₂O (0.2 mL), then was concentrated in vacuum (25° C.). The residue was purified by prep-HPLC column: Waters Xbridge BEH C18 100*30 mm*10 um; mobile phase: [water (NH₄HCO₃)-ACN]; B %: 30%-60%, 8 min to give (1R,2S,5S)-N—((S)-1-cyano-2-((R)-5-oxo-4-azaspiro[2.4]heptan-6-yl)ethyl)-3-((S)-3-methoxy-3-methyl-2-(2,2,2-trifluoroacetamido)butanoyl)-6,6-dimethyl-3-azabicyclo[3.1.0]hexane-2-carboxamide (64.86 mg, 115.66 umol, 28.14% yield, 96.57% purity) was obtained as a white solid. MS (ESI) m/z 542.2 [M+H]⁺.

¹H NMR (400 MHz, DMSO-d₆) δ=9.49-9.23 (m, 1H), 9.22 (s, 1H), 7.89-7.67 (m, 1H), 4.99-4.74 (m, 1H), 4.68-4.59 (m, 1H), 4.17 (s, 1H), 4.03-3.41 (m, 2H), 3.15 3.07 (m, 3H), 2.72-2.57 (m, 1H), 2.28-2.14 (m, 1H), 2.03-1.90 (m, 2H), 1.87-1.76 (m, 1H), 1.61-1.42 (m, 1H), 1.38-1.30 (m, 1H), 1.27-0.87 (m, 12H), 0.79-0.71 (m, 1H), 0.63-0.50 (m, 3H).

Example 26: Synthesis of (3,3,3-trifluoro-2,2-dimethyl-propyl)N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl-propyl]carbamate (Compound 269)

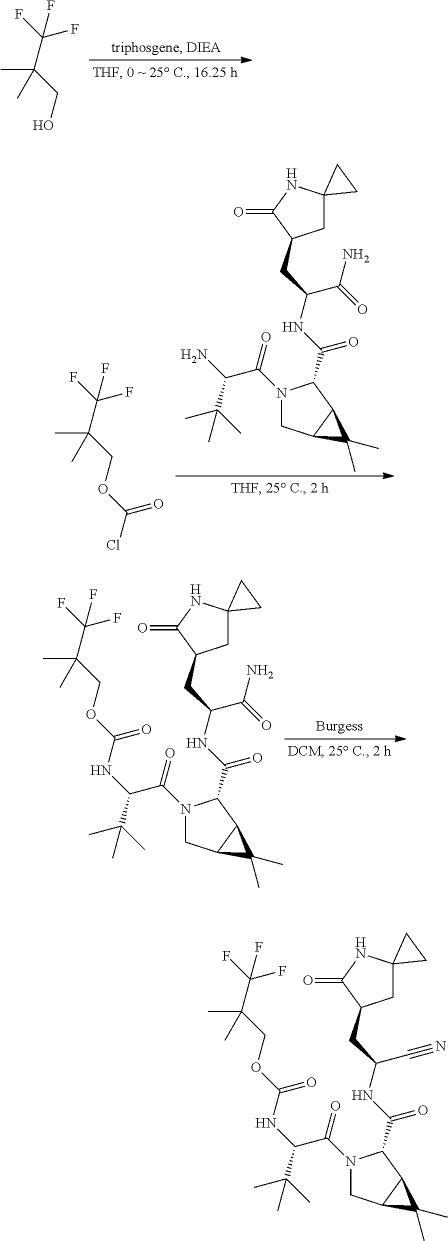

Step 1: (3,3,3-trifluoro-2,2-dimethyl-propyl) carbonochloridate

To a mixture of 3,3,3-trifluoro-2,2-dimethyl-propan-1-ol (200 mg, 1.41 mmol, 1 eq) in THF (3 mL) was added DIEA (363.75 mg, 2.81 mmol, 490.23 uL, 2 eq) at 0° C. Bis (trichloromethyl) carbonate (593.00 mg, 2.00 mmol, 1.42 eq) in THE (3 mL) was added at 0° C. The mixture was stirred at 0° C. for 15 min and then heated to 25° C. and stirred for 16 h. Upon completion, the mixture was concentration in vacuum to give (3,3,3-trifluoro-2,2-dimethyl-propyl) carbonochloridate (270 mg, 1.32 mmol, 93.79% yield) as a white solid.

Step 2: (3,3,3-trifluoro-2,2-dimethyl propyl)N-[(1S)-1-[(1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl propyl]carbamate

To a solution of (1R,2S,5S)-3-[(2S)-2-amino-3,3-dimethyl-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide (370 mg, 826.69 umol, 1 eq) and (3,3,3-trifluoro-2,2-dimethyl-propyl) carbonochloridate (169.12 mg, 826.69 umol, 1 eq) in THE (5 mL) was added DIEA (320.53 mg, 2.48 mmol, 431.98 uL, 3 eq) at 25° C., and then the mixture was stirred at 25° C. for 4 h. After stirring the mixture at 25° C. for 12 h, the reaction mixture was quenched by H₂O (5 ml), extracted with EtOAc (5 ml*2). The combined organic layers were washed with brine (5 mL*2), dried over Na₂SO₄, filtered and concentrated under reduced pressure to give (3,3,3-trifluoro-2,2-dimethyl-propyl)N-[(1S)-1-[(1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl-propyl]carbamate (406 mg, 659.43 umol, 79.77% yield) as a yellow oil.

Step 3: (3,3,3-trifluoro-2,2-dimethyl propyl)N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl-propyl]carbamate

A solution of (3,3,3-trifluoro-2,2-dimethyl-propyl)N-[(1S)-1-[(1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl-propyl]carbamate (400 mg, 649.68 umol, 1 eq) and Burgess reagent (464.48 mg, 1.95 mmol, 3 eq) in DCM (5 mL) was stirred at 25° C. for 2 h. Upon completion, the reaction was quenched with water (0.1 mL), and was blowed to dry with N₂. The residue was purified by prep-HPLC column: Waters Xbridge Prep OBD C18 150*40 mm*10 um; mobile phase: [water (NH₄HCO₃)-ACN]; B %: 45%-75%, 8 min to give (3,3,3-trifluoro-2,2-dimethyl-propyl)N-[(1S)-1 [(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl-propyl]carbamate (36.08 mg, 59.46 umol, 9.15% yield, 98.5% purity) as a white solid. MS (ESI) m/z 598.2 [M+H]⁺.

¹H NMR (400 MHz, DMSO-d₆) δ=9.00 (br d, J=8.5 Hz, 1H), 7.73 (s, 1H), 7.39-7.23 (m, 1H), 5.00-4.88 (m, 1H), 4.19-4.02 (m, 3H), 3.96-3.75 (m, 3H), 2.65-2.60 (m, 1H), 2.25-2.14 (m, 1H), 2.02-1.90 (m, 2H), 1.80 (ddd, J=5.3, 10.8, 13.4 Hz, 1H), 1.57-1.51 (m, 1H), 1.27 (d, J=7.5 Hz, 1H), 1.08 (s, 6H), 1.02 (s, 3H), 0.93 (s, 9H), 0.85 (s, 3H), 0.76-0.69 (m, 1H), 0.60-0.48 (m, 3H)

Example 27: Synthesis of (2,2,3,3-tetrafluorocyclobutyl)N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl-propyl]carbamate (Compound 273)

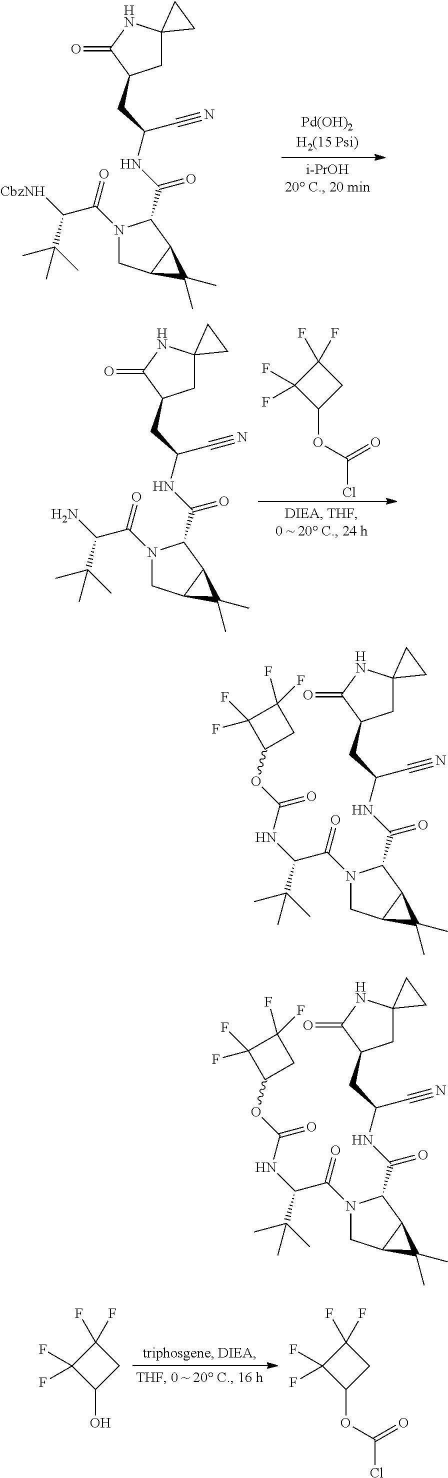

Step 1: (2,2,3,3-tetrafluorocyclobutyl) carbonochloridate

A solution of 2,2,3,3-tetrafluorocyclobutanol (150 mg, 1.04 mmol, 1 eq) in THE (3 mL) was added DIEA (269.13 mg, 2.08 mmol, 362.71 uL, 2 eq), and a solution of bis(trichloromethyl) carbonate (423.29 mg, 1.43 mmol, 1.37 eq) in THE (3 mL) was added dropwise into the former solution at 0° C. The mixture was stirred at 20° C. for 16 h. Upon completion, the reaction mixture was concentrated under reduced pressure to give (2,2,3,3-tetrafluorocyclobutyl) carbonochloridate (200 mg, crude) as a yellow solid.

Step 2: (1R,2S,5S)-3-[(2S)-2-amino-3,3-dimethyl-butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide

A solution of benzyl N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl-propyl]carbamate (300 mg, 532.21 umol, 1 eq) in isopropanol (2 mL) was added Pd(OH)₂ (235.43 mg, 335.29 umol, 20% purity, 0.63 eq) under N₂. The suspension was degassed under vacuum and purged with H₂ (3 times). The mixture was stirred under H₂ (15 Psi) at 20° C. for 20 min. Upon completion, the reaction mixture was filtered and concentrated under reduced pressure to give (1R,2S,5S)-3-[(2S)-2-amino-3,3-dimethyl-butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (220 mg, crude) as a white solid. MS (ESI) m/z 430.4 [M+H]⁺.

Step 3: (2,2,3,3-tetrafluorocyclobutyl)N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl propyl]carbamate

A solution of (2,2,3,3-tetrafluorocyclobutyl) carbonochloridate (190 mg, 920.00 umol, 1.9 eq) in THE (2 mL) was added DIEA (187.74 mg, 1.45 mmol, 253.02 uL, 3 eq) at 0° C. A solution of (1R,2S,5S)-3-[(2S)-2-amino-3,3-dimethyl-butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (207.99 mg, 484.21 umol, 1 eq) in THE (2 mL) was added dropwise into the former solution at 0° C., and then the mixture was stirred at 20° C. for 24 h. Upon completion, the reaction mixture was quenched with water (5 mL) and extracted with EA (5 mL*3), dried over Na₂SO₄, filtered and concentrated under reduced pressure to give a residue. The residue was purified by prep-HPLC (column: Phenomenex C18 75*30 mm*3 um; mobile phase: [water(NH₄HCO₃)-ACN]; B %: 45%-65%, 8 min) to give (2,2,3,3-tetrafluorocyclobutyl)N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl-propyl]carbamate Isomer 1 (13.21 mg, 21.88 umol, 4.52% yield, 99.3% purity) as a white solid. MS (ESI) m/z 600.2 [M+H]⁺.

¹H NMR (400 MHz, DMSO-d₆) δ=9.02 (d, J=8.5 Hz, 1H), 7.88 (d, J=9.1 Hz, 1H), 7.73 (s, 1H), 5.29-5.10 (m, 1H), 4.99-4.86 (m, 1H), 4.17 (s, 1H), 4.07 (d, J=9.2 Hz, 1H), 3.91-3.74 (m, 2H), 3.24-3.08 (m, 1H), 2.66-2.61 (m, 1H), 2.24-2.21 (m, 1H), 2.03 1.88 (m, 2H), 1.84-1.80 (m, 1H), 1.55-1.53 (m, 1H), 1.38-1.05 (m, 2H), 1.04-0.79 (m, 15H), 0.76-0.68 (m, 1H), 0.53 (s, 3H)

(2,2,3,3-Tetrafluorocyclobutyl)N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl-propyl]carbamate Isomer 2 (13.19 mg, 20.61 umol, 4.26% yield, 93.7% purity) was obtained as a white solid. MS (ESI) m/z 600.2 [M+H]⁺.

¹H NMR (400 MHz, DMSO-d₆) δ=9.00 (br d, J=8.5 Hz, 1H), 7.93-7.81 (m, 1H), 7.80-7.67 (m, 1H), 5.25-5.11 (m, 1H), 4.95-4.89 (m, 1H), 4.20-4.13 (m, 1H), 4.06 (br d, J=8.6 Hz, 1H), 3.90-3.81 (m, 1H), 3.81-3.69 (m, 1H), 3.24-3.12 (m, 1H), 2.65 2.60 (m, 1H), 2.24-2.14 (m, 1H), 2.01-1.88 (m, 2H), 1.83-1.76 (m, 1H), 1.58-1.50 (m, 1H), 1.40-1.12 (m, 2H), 1.05-0.84 (m, 15H), 0.82-0.72 (m, 1H), 0.71-0.44 (m, 3H)

Example 28: Synthesis of 1-(trifluoromethyl)cyclobutyl ((S)-1-((1R,2S,5S)-2-(((S)-1-cyan-2-((R)-5-oxo-4-azaspiro[2.4]heptan-6-yl)ethyl)carbamoyl)-6,6-dimethyl-3-azabicyclo[3.1.0]hexan-3-yl)-3,3-dimethyl-1-oxobutan-2-yl)carbamate (Compound 274)

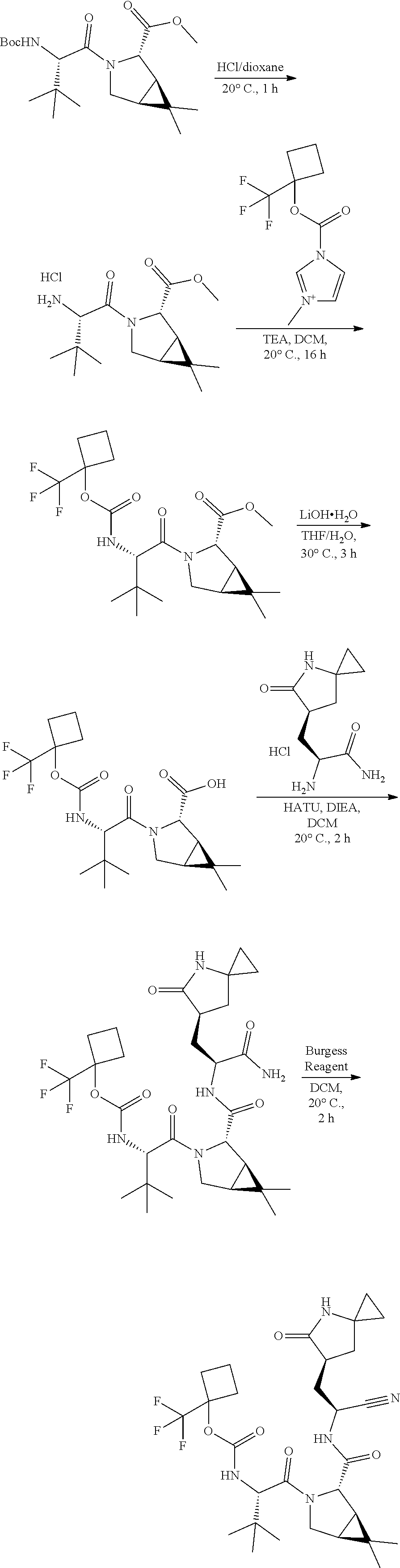

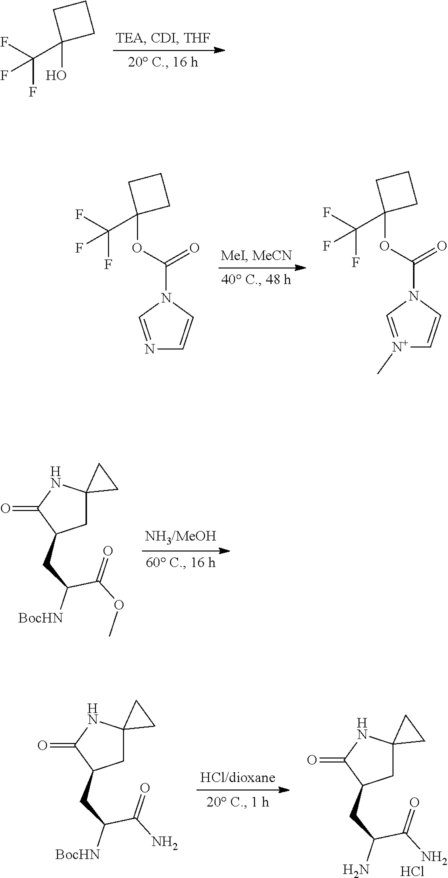

Step 1: [1-(trifluoromethyl) cyclobutyl]imidazole-1-carboxylate

To a solution of 1-(trifluoromethyl) cyclobutanol (250 mg, 1.78 mmol, 1 eq) in THE (8 mL) was added TEA (541.68 mg, 5.35 mmol, 745.09 uL, 3 eq) and CDI (318.27 mg, 1.96 mmol, 1.1 eq), and then the mixture was stirred at 20° C. for 16 h. Upon completion, the mixture was concentrated in vacuum to obtained [1-(trifluoromethyl) cyclobutyl] imidazole-1-carboxylate (400 mg, crude) as a white gum.

Step 2: [1-(trifluoromethyl)cyclobutyl]3-methylimidazol-3-ium-1-carboxylate

A solution of [1-(trifluoromethyl)cyclobutyl] imidazole-1-carboxylate (400.00 mg, 1.71 mmol, 1 eq) in ACN (8 mL) was added with Mel (2.91 g, 20.50 mmol, 1.28 mL, 12 eq), and then the mixture was stirred at 40° C. for 48 h. The resulting mixture was concentrated in vacuum to obtain [1-(trifluoromethyl)cyclobutyl] 3-methylimidazol-3-ium-1-carboxylate (400 mg, crude) as a red gum.

Step 3: tert-butyl N-[(1S)-2-amino-2-oxo-1-[[(6R)-5-oxo-4-azaspiro[2.4]heptan-6-yl]methyl]ethyl]carbamate

A solution of methyl (2S)-2-(tert-butoxycarbonylamino)-3-[(6R)-5-oxo-4-azaspiro[2.4]heptan-6-yl]propanoate (300 mg, 960.43 umol, 1 eq) in NH₃/MeOH (10 mL, 7 M) was stirred at 60° C. for 16 h. Upon completion, the mixture was concentrated in vacuum to obtain tert-butyl N-[(1S)-2-amino-2-oxo-1-[[(6R)-5-oxo-4-azaspiro[2.4]heptan-6-yl]methyl]ethyl]carbamate (250 mg, crude) as a light yellow solid.

Step 4: (2S)-2-amino-3-[(6R)-5-oxo-4-azaspiro[2.4]heptan-6-yl]propanamide

A solution of tert-butyl N-[(1S)-2-amino-2-oxo-1-[[(6R)-5-oxo-4-azaspiro[2.4]heptan-6-yl]methyl]ethyl]carbamate (250 mg, 840.76 umol, 1 eq) in HCl/dioxane (5 mL, 4 M) was stirred at 20° C. for 1 h. Upon completion, the mixture was concentrated in vacuum to obtain (2S)-2-amino-3-[(6R)-5-oxo-4-azaspiro[2.4]heptan-6-yl]propanamide (200 mg, crude, HCl) as a white solid.

Step 5: methyl (1R,2S,5S)-3-[(2S)-2-amino-3,3-dimethyl-butanoyl]-6,6-dimethyl-3-azabicyclo[3.1.0]hexane-2-carboxylate

A solution of methyl (1R,2S,5S)-3-[(2S)-2-(tert-butoxycarbonylamino)-3,3-dimethyl-butanoyl]-6,6-dimethyl-3-azabicyclo[3.1.0]hexane-2-carboxylate (50 mg, 130.72 umol, 1 eq) in HCl/MeOH (3 mL, 4 M) was stirred at 20° C. for 1 h. Upon completion, the mixture was concentrated in vacuum to obtain methyl (1R,2S,5S)-3-[(2S)-2-amino-3,3-dimethyl-butanoyl]-6,6-dimethyl-3-azabicyclo[3.1.0]hexane-2-carboxylate (40 mg, crude, HCl) as a white solid.

Step 6: methyl (1R,2S,5S)-3-[(2S)-3,3-dimethyl-2-[[1-(trifluoromethyl)cyclobutoxy]carbonylamino]butanoyl]-6,6-dimethyl-3-azabicyclo[3.1.0]hexane-2-carboxylate

To a solution of methyl (1R,2S,5S)-3-[(2S)-2-amino-3,3-dimethyl-butanoyl]-6,6-dimethyl-3-azabicyclo[3.1.0]hexane-2-carboxylate (300 mg, 940.92 umol, 1 eq, HCl) in DCM (5 mL) was added TEA (285.63 mg, 2.82 mmol, 392.89 uL, 3 eq). The solution was added with [l-(trifluoromethyl)cyclobutyl] 3-methylimidazol-3-ium-1-carboxylate (398.62 mg, 1.60 mmol, 1.7 eq), and then the mixture was stirred at 20° C. for 16 h. Upon completion, the mixture was quenched by water (10 mL) and extracted with DCM (4 mL*3). The organic phase was concentrated in vacuum. The crude product was purified by column (SiO₂, PE:EA=1:0 to 2:1) to afford methyl (1R,2S,5S)-3-[(2S)-3,3-dimethyl-2-[[1-(trifluoromethyl)cyclobutoxy]carbonylamino]butanoyl]-6,6-dimethyl-3-azabicyclo[3.1.0]hexane-2-carboxylate (400 mg, 624.34 umol, 66.35% yield, 70% purity) as a colourless gum.

Step 7: (1R,2S,5S)-3-[(2S)-3,3-dimethyl-2-[[1-(trifluoromethyl)cyclobutoxy]carbonylamino]butanoyl]-6,6-dimethyl-3-azabicyclo[3.1.0]hexane-2-carboxylic acid

To a solution of methyl (1R,2S,5S)-3-[(2S)-3,3-dimethyl-2-[[1-(trifluoromethyl)cyclobutoxy]carbonylamino]butanoyl]-6,6-dimethyl-3-azabicyclo[3.1.0]hexane-2-carboxylate (400 mg, 624.34 umol, 70% purity, 1 eq) in THE (4 mL) and H₂O (2 mL) was added LiOH.H₂O (157.20 mg, 3.75 mmol, 6 eq). The resulting mixture was stirred at 30° C. for 3 h. Upon completion, the mixture was concentrated in vacuum and was adjusted pH-1 with 1M HCl and was extracted with EA (10 mL*3), and then the organic phase was dried with Na₂SO₄, filtered and concentrated in vacuum to obtain (1R,2S,5S)-3-[(2S)-3,3-dimethyl-2-[[1-(trifluoromethyl)cyclobutoxy]carbonylamino]butanoyl]-6,6-dimethyl-3-azabicyclo[3.1.0]hexane-2-carboxylic acid (300 mg, crude) as a colorless gum.

Step 8: [1-(trifluoromethyl)cyclobutyl]N-[(1S)-1-((1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl propyl]carbamate

To a solution of (1R,2S,5S)-3-[(2S)-3,3-dimethyl-2-[[1-(trifluoromethyl)cyclobutoxy]carbonylamino]butanoyl]-6,6-dimethyl-3-azabicyclo[3.1.0]hexane-2-carboxylic acid (300 mg, 690.53 umol, 1 eq) and (2S)-2-amino-3-[(6R)-5-oxo-4-azaspiro[2.4]heptan-6-yl]propanamide (161.37 mg, 690.53 umol, 1 eq, HCl) in DCM (8 mL) was added DIEA (267.73 mg, 2.07 mmol, 360.83 uL, 3 eq) and HATU (262.56 mg, 690.53 umol, 1 eq). The resulting mixture was stirred at 20° C. for 2 h. Upon completion, the mixture was quenched by water (15 mL) and was extracted with DCM (5 mL*3), the organic phase was washed with sat. NaCl (4 mL), dried with Na₂SO₄, filtered and concentrated in vacuum to give the crude product. The crude product was purified by column (SiO₂, PE:EA=1:1 to EA:MeOH=15:1 to EA:MeOH=5:1) to obtain [1-(trifluoromethyl)cyclobutyl] N-[(1S)-1-[(1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl-propyl]carbamate (200 mg, 228.14 umol, 33.04% yield, 70% purity) as a light yellow gum. MS (ESI) m/z 614.4 [M+H]⁺

Step 9: [1-(trifluoromethyl)cyclobutyl]N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl propyl]carbamate

To a solution of [1-(trifluoromethyl)cyclobutyl] N-[(1S)-1-[(1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl-propyl]carbamate (170 mg, 277.02 umol, 1 eq) in DCM (3 mL) was added Burgess reagent (198.05 mg, 831.07 umol, 3 eq), and the resulting mixture was stirred at 20° C. for 2 h. Upon completion, the mixture was quenched by water (5 mL) and then was extracted with DCM (3 mL*3). The organic phase was dried with Na₂SO₄, filtered and concentrated in vacuum. The crude product was purified by prep-HPLC (column: Waters Xbridge Prep OBD C18 150*40 mm*10 um; mobile phase: [water (NH₄HCO₃)-ACN]; B %: 45%-75%, 8 min) to afford [1-(trifluoromethyl)cyclobutyl]N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl-propyl]carbamate (4 mg, 6.51 umol, 2.35% yield, 96.9% purity) as a white solid. MS (ESI) m/z 596.2 [M+H]⁺

¹H NMR (400 MHz, MeOD-d₄) δ=5.06-4.97 (m, 1H), 4.31-4.25 (m, 1H), 4.21-4.15 (m, 1H), 4.02-3.91 (m, 2H), 3.57-3.45 (m, 1H), 2.93-2.80 (m, 2H), 2.75-2.62 (m, 2H), 2.58-2.46 (m, 1H), 2.43-2.32 (m, 1H), 2.22-2.12 (m, 1H), 2.06 (dd, J=9.2, 12.6 Hz, 1H), 1.90-1.79 (m, 1H), 1.66-1.59 (m, 1H), 1.42-1.36 (m, 1H), 1.30 (br s, 1H), 1.11 1.07 (m, 3H), 1.02 (s, 9H), 0.96-0.93 (m, 3H), 0.90-0.58 (m, 4H).

Example 29: Synthesis of (1-cyclopropyl-2,2,2-trifluoro-ethyl)N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl-propyl]carbamate (Compound 275)

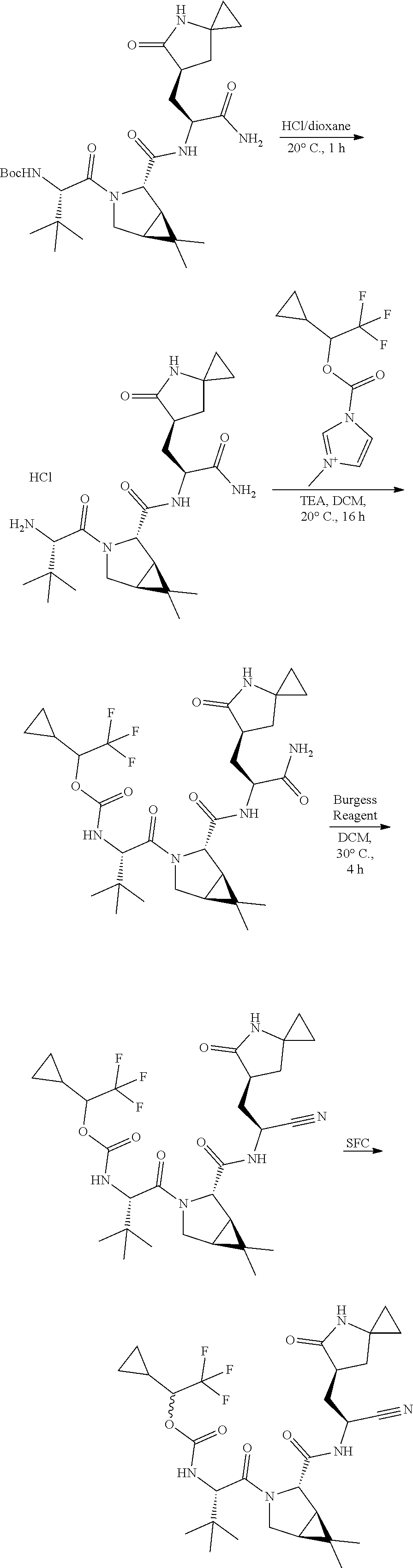

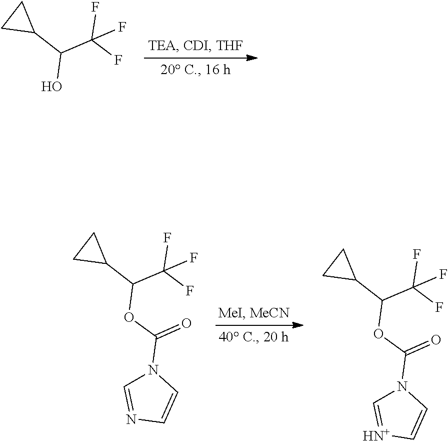 [SMILES: OC(C1CC1)C(F)(F)F ; O=C(OC(C1CC1)C(F)(F)F)n1ccnc1 ; O=C(OC(C1CC1)C(F)(F)F)n1cc[nH+]c1]

Step 1: (1-cyclopropyl-2,2,2-trifluoro-ethyl) imidazole-1-carboxylate

To a solution of 1-cyclopropyl-2,2,2-trifluoro-ethanol (250 mg, 1.78 mmol, 1 eq) in THE (3 mL) was added CDI (318.27 mg, 1.96 mmol, 1.1 eq) and TEA (541.68 mg, 5.35 mmol, 745.09 uL, 3 eq), and the mixture was stirred at 20° C. for 16 h. Upon completion, the mixture was concentrated in vacuum to obtain (1-cyclopropyl-2,2,2-trifluoro-ethyl) imidazole-1-carboxylate (400 mg, crude) as a white gum.

Step 2: (1-cyclopropyl-2,2,2-trifluoro-ethyl) 3-methylimidazol-3-ium-1-carboxylate

To a solution of (1-cyclopropyl-2,2,2-trifluoro-ethyl) imidazole-1-carboxylate (400 mg, 1.71 mmol, 1 eq) in ACN (8 mL) was added Mel (2.91 g, 20.50 mmol, 1.28 mL, 12 eq), and the mixture was stirred at 40° C. for 20 h. Upon completion, the mixture was concentrated in vacuum to afford (1-cyclopropyl-2,2,2-trifluoro-ethyl) 3-methylimidazol-3-ium-1-carboxylate (400 mg, crude) as a red gum.

Step 3: (1R,2S,5S)-3-[(2S)-2-amino-3,3-dimethyl-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide

A solution of tert-butyl N-[(1S)-1-[(1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl-propyl]carbamate (0.3 g, 547.76 umol, 1 eq) in HCl/dioxane (5 mL, 4 M) was stirred at 20° C. for 1 h. Upon completion, the mixture was concentrated in vacuum to obtain (1R,2S,5S)-3-[(2S)-2-amino-3,3-dimethyl-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide (200 mg, crude, HCl) as a light yellow solid. MS (ESI) m/z 448.2 [M+H]⁺

Step 4: (S)-5-((S)-2-((tert-butoxycarbonyl)amino)-3,3-dimethylbutanoyl)-5-azaspiro[2.4]heptane-6-carboxylic acid

To a solution of (1R,2S,5S)-3-[(2S)-2-amino-3,3-dimethyl-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide (200 mg, 413.20 umol, 1 eq, HCl) in DCM (4 mL) was added TEA (125.43 mg, 1.24 mmol, 172.54 uL, 3 eq), followed by the addition of (1-cyclopropyl-2,2,2-trifluoro-ethyl) 3-methylimidazol-3-ium-1-carboxylate (308.92 mg, 1.24 mmol, 3 eq). The resulting mixture was stirred at 20° C. for 16 h. Upon completion, the mixture was concentrated in vacuum. The crude product was purified by column (SiO₂, PE:EA=1:1 to EA to EA:MeOH=10:1) and the purified product was poured into water (20 mL) and was extracted with EA (10 mL*3), washed with sat. NaCl (1 mL), dried with Na₂SO₄, filtered and concentrated in vacuum to obtain N-[(1S)-1-[(1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl-propyl]carbamate (200 mg, 195.55 umol, 47.33% yield, 60% purity) as a yellow gum. MS (ESI) m/z 614.4 [M+H]⁺

Step 5: (1-cyclopropyl-2,2,2-trifluoro-ethyl)N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl propyl]carbamate

To a solution of (1-cyclopropyl-2,2,2-trifluoro-ethyl)N-[(1S)-1-[(1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl-propyl]carbamate (200 mg, 325.91 umol, 1 eq) in DCM (3 mL) was added Burgess reagent (233.00 mg, 977.73 umol, 3 eq), and then the mixture was stirred at 30° C. for 4 h. Upon completion, the mixture was quenched by water (15 mL), and was extracted with DCM (5 mL*3). The organic phase was washed with sat. NaCl (3 mL), then was concentrated in vacuum, and the crude product was purified by prep-HPLC (column: Waters Xbridge Prep OBD C18 150*40 mm 10 um; mobile phase: [water (NH₄HCO₃)-ACN]; B %: 40%-75%, 8 min) to obtain (1-cyclopropyl-2,2,2-trifluoro-ethyl)N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl-propyl]carbamate (50 mg, 83.94 umol, 25.76% yield, 100% purity) as a white solid. MS (ESI) m/z 596.3 [M+H]⁺

Step 6: (1-cyclopropyl-2,2,2-trifluoro-ethyl)N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl propyl]carbamate

(1-Cyclopropyl-2,2,2-trifluoro-ethyl)N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl-propyl]carbamate was separated by SFC (column: DAICEL CHIRALPAK AD-H (250 mm*30 mm, Sum); mobile phase: [0.1% NH₃H₂O IPA]; B %: 5%-30%, 16 min) to obtain (1-cyclopropyl-2,2,2-trifluoro-ethyl) N [(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl-propyl]carbamate Isomer 1 (12 mg, 19.52 umol, 25.84% yield, 96.9% purity) as a white solid. MS (ESI) m/z 596.2 [M+H]⁺

¹H NMR (400 MHz, DMSO-d₆) δ=9.01 (d, J=8.6 Hz, 1H), 7.81-7.68 (m, 2H), 4.93 (ddd, J=5.2, 8.4, 10.6 Hz, 1H), 4.74-4.61 (m, 1H), 4.22-4.15 (m, 1H), 4.07 (d, J=9.2 Hz, 1H), 3.91-3.73 (m, 2H), 2.72-2.58 (m, 1H), 2.25-2.13 (m, 1H), 2.04-1.89 (m, 2H), 1.87-1.74 (m, 1H), 1.58-1.49 (m, 1H), 1.28 (d, J=7.6 Hz, 1H), 1.17-1.07 (m, 1H), 0.94-0.78 (m, 16H), 0.77-0.59 (m, 2H), 0.56-0.45 (m, 4H), 0.37-0.26 (m, 1H).

(1-Cyclopropyl-2,2,2-trifluoro-ethyl)N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl-propyl]carbamate Isomer 2 (10 mg, 16.44 umol, 21.76% yield, 97.9% purity) was obtained as a white solid. MS (ESI) m/z 596.2 [M+H]⁺

¹H NMR (400 MHz, DMSO-d₆) δ=9.08-8.91 (m, 1H), 7.93-7.69 (m, 2H), 4.98-4.86 (m, 1H), 4.72-4.59 (m, 1H), 4.15 (s, 1H), 4.09 (d, J=9.2 Hz, 1H), 3.89-3.80 (m, 1H), 3.79-3.72 (m, 1H), 2.69-2.59 (m, 1H), 2.25-2.14 (m, 1H), 2.03-1.89 (m, 2H), 1.86-1.73 (m, 1H), 1.58-1.45 (m, 1H), 1.30-1.24 (m, 1H), 1.19-1.08 (m, 1H), 0.94-0.77 (m, 16H), 0.76-0.69 (m, 1H), 0.68-0.61 (m, 1H), 0.59-0.49 (m, 4H), 0.44-0.34 (m, 1H).

Example 29: Synthesis of 2-fluoroethyl N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl-propyl]carbamate (Compound 278)

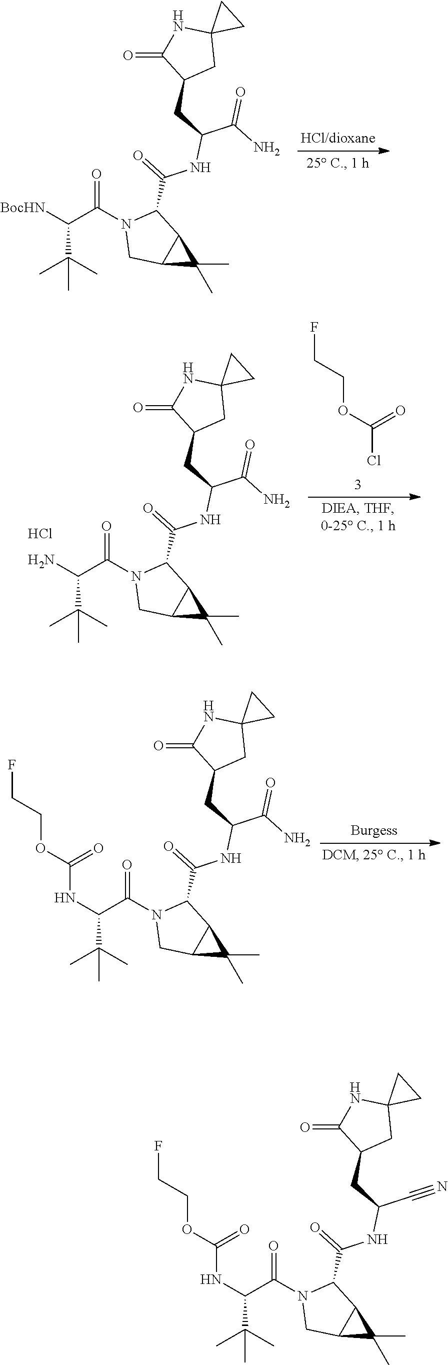 [SMILES: CC(C)(C)OC(=O)N[C@H](C(=O)N1C[C@H]2[C@@H]([C@H]1C(=O)N[C@@H](C[C@@H]1CC3(CC3)NC1=O)C(N)=O)C2(C)C)C(C)(C)C ; Cl ; CC(C)(C)[C@H](N)C(=O)N1C[C@H]2[C@@H]([C@H]1C(=O)N[C@@H](C[C@@H]1CC3(CC3)NC1=O)C(N)=O)C2(C)C ; O=C(Cl)OCCF ; CC(C)(C)[C@H](NC(=O)OCCF)C(=O)N1C[C@H]2[C@@H]([C@H]1C(=O)N[C@@H](C[C@@H]1CC3(CC3)NC1=O)C(N)=O)C2(C)C ; CC(C)(C)[C@H](NC(=O)OCCF)C(=O)N1C[C@H]2[C@@H]([C@H]1C(=O)N[C@H](C#N)C[C@@H]1CC3(CC3)NC1=O)C2(C)C]

Step 1: (1R,2S,5S)-3-[(2S)-2-amino-3,3-dimethyl-butanoyl]-N-[(1S)-2-amino-2-oxo-1-[[(6R)-5-oxo-4-azaspiro[2.4]heptan-6-yl/methyl/ethyl]-6,6-dimethyl-3-azabicyclo[3.1.0]hexane-2-carboxamide

A solution of tert-butyl N-[(1S)-1-[(1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl-propyl]carbamate (250 mg, 456.47 umol, 1 eq) in HCl/dioxane (456.47 umol, 5 mL, 1 eq) was stirred at 25° C. for 1 h. Upon completion, the reaction mixture was concentrated under reduced pressure to give (1R,2S,5S)-3-[(2S)-2-amino-3,3-dimethyl-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide (215 mg, crude, HCl) as a white solid.

Step 2: 2-fluoroethyl N-[(1S)-1-[(1R,2S,5S)-2-[((1S)-2-amino-2-oxo-1-[[(6R)-5-oxo-4-azaspiro[2.4]heptan-6-yl/methyl/ethyl]carbamoyl]-6,6-dimethyl-3-azabicyclo[3.1.0]hexane-3-carbonyl]-2,2-dimethyl propyl/carbamate

To a solution of (1R,2S,5S)-3-[(2S)-2-amino-3,3-dimethyl-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide (215 mg, 444.19 umol, 1 eq, HCl) in THE (5 mL) was added DIEA (172.22 mg, 1.33 mmol, 232.11 uL, 3 eq). 2-fluoroethyl carbonochloridate (84.29 mg, 666.28 umol, 62.91 uL, 1.5 eq) in THE (1 mL) was then added at 0° C. The mixture was stirred at 25° C. for 1 h. Upon completion, the reaction mixture was poured into H₂O 30 mL at 25° C., and then extracted with DCM (30 mL*3). The combined organic layers were washed with brine 30 mL, dried over Na₂SO₄, filtered and concentrated under reduced pressure to give a residue. The residue was purified by column chromatography (SiO₂, DCM:MeOH=100:0 to 90:10) to give 2-fluoroethyl N-[(1S)-1-[(1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl-propyl]carbamate (171 mg, 273.22 umol, 61.51% yield, 85.9% purity) as a yellow oil. MS (ESI) m/z 538.4 [M+H]⁺.

Step 3: 2-fluoroethyl N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl propyl]carbamate

To a solution of 2-fluoroethyl N-[(1S)-1-[(1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl-propyl]carbamate (171 mg, 318.07 umol, 1 eq) in DCM (3 mL) was added Burgess reagent (189.49 mg, 795.17 umol, 2.5 eq). The mixture was stirred at 25° C. for 1 h. Upon completion, the reaction mixture was poured into H₂O 30 mL at 25° C., and then extracted with DCM (30 mL*3). The combined organic layers were washed with brine 30 mL, dried over Na₂SO₄, filtered and concentrated under reduced pressure to give a residue. The residue was purified by prep-HPLC (column: Waters Xbridge Prep OBD C18 150*40 mm*10 um; mobile phase: [water(NH₄HCO₃)-ACN]; B %: 30%-60%, 8 min) to give 2-fluoroethyl N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl-propyl]carbamate (39.15 mg, 75.35 umol, 23.69% yield, 100% purity) as a white solid. MS (ESI) m/z 520.3 [M+H]⁺.

¹H NMR (400 MHz, DMSO-d₆) δ=9.00 (d, J=8.4 Hz, 1H), 7.74 (s, 1H), 7.34 (d, J=8.4 Hz, 1H), 5.00-4.86 (m, 1H), 4.70-4.43 (m, 2H), 4.25-4.08 (m, 3H), 4.02 (d, J=8.4 Hz, 1H), 3.92-3.72 (m, 2H), 2.71-2.56 (m, 1H), 2.25-2.12 (m, 1H), 2.02-1.87 (m, 2H), 1.86-1.72 (m, 1H), 1.61-1.45 (m, 1H), 1.27 (d, J=7.6 Hz, 1H), 1.06-0.99 (m, 3H), 0.94 (s, 9H), 0.88 (s, 3H), 0.76-0.70 (m, 1H), 0.60-0.48 (m, 3H).

Example 30: Synthesis of (1R,2S,5S)—N-[(1S)-1-cyan-2-[(6R)-5-oxo-4-azaspiro[2.4]heptan-6-yl]ethyl]-3-[(2S)-2-[[ethyl(2,2,2-trifluoroethyl)carbamoyl]amino]-3,3-dimethyl-butanoyl]-6,6-dimethyl-3-azabicyclo[3.1.0]hexane-2-carboxamide (Compound 279)

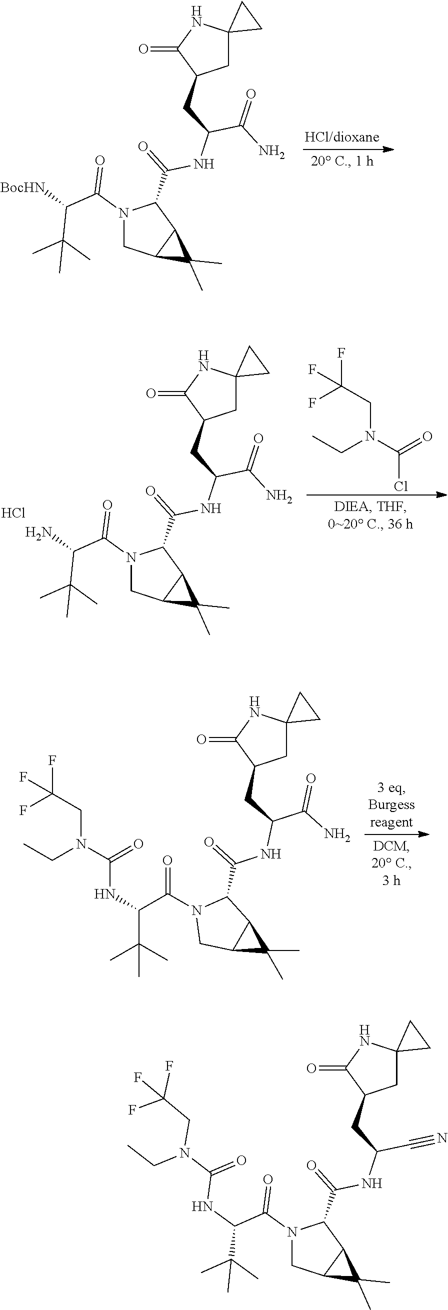

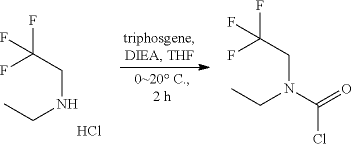

Step 1: N-ethyl-N-(2,2,2-trifluoroethyl)carbamoyl chloride

A solution of N-ethyl-2,2,2-trifluoro-ethanamine (0.2 g, 1.57 mmol, 1 eq) in THE (4 mL) was added DIEA (406.71 mg, 3.15 mmol, 548.13 uL, 2 eq). and then a solution of triphosgene (639.69 mg, 2.16 mmol, 1.37 eq) in THE (4 mL) was dropwise added into the former solution at 0° C. The mixture was stirred at 25° C. for 2 h. Upon completion, the reaction mixture was concentrated under reduced pressure to give N-ethyl-N-(2,2,2-trifluoroethyl)carbamoyl chloride (210 mg, crude) as a yellow solid.

Step 2: (1R,2S,5S)-3-[(2S)-2-amino-3,3-dimethyl-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide

A solution of tert-butyl N-[(1S)-1-[(1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl-propyl]carbamate (500 mg, 912.93 umol, 1 eq) in HCl/dioxane (4 M, 5 mL, 21.91 eq) was stirred at 20° C. for 1 h. Upon completion, the reaction mixture was concentrated under reduced pressure to give (1R,2S,5S)-3-[(2S)-2-amino-3,3-dimethyl-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide (400 mg, crude, HCl) as a yellow solid.

Step 3: (1R,2S,5S)—N-[(1S)-2-amino-2-oxo-1-[[(6R)-5-oxo-4-azaspiro[2.4]heptan-6-yl]methyl]ethyl]-3-[(2S)-2-[[ethyl(2,2,2-trifluoroethyl)carbamoyl]amino]-3,3-dimethyl-butanoyl]-6,6-dimethyl-3-azabicyclo[3.1.0]hexane-2-carboxamide

A solution of (1R,2S,5S)-3-[(2S)-2-amino-3,3-dimethyl-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide (400 mg, 661.12 umol, 80% purity, 1 eq, HCl) in THE (2 mL) was added DIEA (256.33 mg, 1.98 mmol, 345.46 uL, 3 eq) at 0° C., and then a solution of N-ethyl-N-(2,2,2-trifluoroethyl)carbamoyl chloride (187.98 mg, 991.67 umol, 1.5 eq) in THE (2 mL) was dropwise added into the former solution at 0° C. The resulting mixture was stirred at 20° C. for 36 h. Upon completion, the reaction mixture was quenched with water (5 mL) and extracted with DCM (5 mL*3), dried over Na₂SO₄, filtered and concentrated under reduced pressure to give (1R,2S,5S)—N-[(1S)-2-amino-2-oxo-1-[[(6R)-5-oxo-4-azaspiro[2.4]heptan-6-yl]methyl]ethyl]-3-[(2S)-2-[[ethyl(2,2,2-trifluoroethyl)carbamoyl]amino]-3,3-dimethyl-butanoyl]-6,6-dimethyl-3-azabicyclo[3.1.0]hexane-2-carboxamide (150 mg, 237.23 umol, 35.88% yield, 95% purity) as a white solid.

Step 4: (1R,2S,5S)—N-[(1S)-1-cyano-2-[(6R)-5-oxo-4-azaspiro[2.4]heptan-6-yl]ethyl]-3-[(2S)-2-[[ethyl(2,2,2-trifluoroethyl)carbamoyl]amino]-3,3-dimethyl-butanoyl]-6,6-dimethyl-3-azabicyclo[3.1.0]hexane-2-carboxamide

A solution of (1R,2S,5S)—N-[(1S)-2-amino-2-oxo-1-[[(6R)-5-oxo-4-azaspiro[2.4]heptan-6-yl]methyl]ethyl]-3-[(2S)-2-[[ethyl(2,2,2-trifluoroethyl)carbamoyl]amino]-3,3-dimethyl-butanoyl]-6,6-dimethyl-3-azabicyclo[3.1.0]hexane-2-carboxamide (140 mg, 186.46 umol, 80% purity, 1 eq) in DCM (4 mL) was added with Burgess reagent (133.30 mg, 559.37 umol, 3 eq) and stirred at 20° C. for 3 h. Upon completion, the reaction mixture was quenched with water (1 mL) and concentrated under reduced pressure to give a residue. The residue was purified by prep-HPLC(column: Phenomenex C18 80*40 mm*3 um; mobile phase: [water(NH₄HCO₃)-ACN]; B %: 30%-50%, 8 min) to give (1R,2S,5S)—N-[(1S)-1-cyano-2-[(6R)-5-oxo-4-azaspiro[2.4]heptan-6-yl]ethyl]-3-[(2S)-2-[[ethyl(2,2,2-trifluoroethyl)carbamoyl]amino]-3,3-dimethyl-butanoyl]-6,6-dimethyl-3-azabicyclo[3.1.0]hexane-2-carboxamide (47.62 mg, 81.73 umol, 43.83% yield, 100% purity) as a white solid. MS (ESI) m/z 583.2 [M+H]⁺.

¹H NMR (400 MHz, DMSO-d₆) δ=8.99 (d, J=8.6 Hz, 1H), 7.83-7.66 (m, 1H), 5.85 (d, J=9.3 Hz, 1H), 5.00-4.86 (m, 1H), 4.28 (d, J=9.1 Hz, 1H), 4.22 (br dd, J=9.7, 15.7 Hz, 1H), 4.15 (s, 1H), 4.00-3.89 (m, 1H), 3.89-3.79 (m, 2H), 3.52-3.40 (m, 1H), 3.29-3.21 (m, 1H), 2.68-2.60 (m, 1H), 2.24-2.15 (m, 1H), 2.03-1.90 (m, 2H), 1.86-1.74 (m, 1H), 1.53 (br dd, J=4.2, 7.4 Hz, 1H), 1.27 (d, J=7.6 Hz, 1H), 1.05-0.99 (m, 6H), 0.95 (s, 9H), 0.83 (s, 3H), 0.73 (br d, J=9.0 Hz, 1H), 0.60-0.49 (m, 3H).

Example 31: Synthesis of (1R,2S,5S)—N-[(1S)-1-cyan-2-[(6R)-5-oxo-4-azaspiro[2.4]heptan-6-yl]ethyl]-3-[(2S)-2-[(3,3-difluoropyrrolidine-1-carbonyl)amino]-3,3-dimethyl-butanoyl]-6,6-dimethyl-3-azabicyclo[3.1.0]hexane-2-carboxamide (Compound 280)

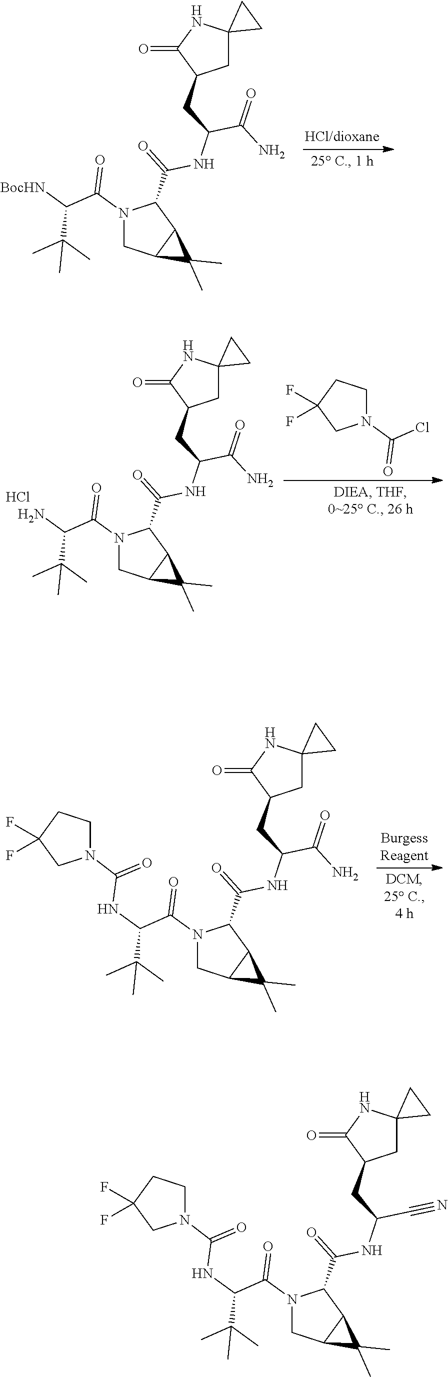 [SMILES: CC(C)(C)OC(=O)N[C@H](C(=O)N1C[C@H]2[C@@H]([C@H]1C(=O)N[C@@H](C[C@@H]1CC3(CC3)NC1=O)C(N)=O)C2(C)C)C(C)(C)C ; Cl ; CC(C)(C)[C@H](N)C(=O)N1C[C@H]2[C@@H]([C@H]1C(=O)N[C@@H](C[C@@H]1CC3(CC3)NC1=O)C(N)=O)C2(C)C ; O=C(Cl)N1CCC(F)(F)C1 ; CC(C)(C)[C@H](NC(=O)N1CCC(F)(F)C1)C(=O)N1C[C@H]2[C@@H]([C@H]1C(=O)N[C@@H](C[C@@H]1CC3(CC3)NC1=O)C(N)=O)C2(C)C ; CC(C)(C)[C@H](NC(=O)N1CCC(F)(F)C1)C(=O)N1C[C@H]2[C@@H]([C@H]1C(=O)N[C@H](C#N)C[C@@H]1CC3(CC3)NC1=O)C2(C)C]

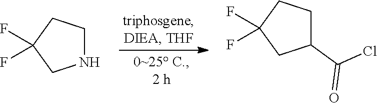 [SMILES: FC1(F)CCNC1 ; O=C(Cl)C1CCC(F)(F)C1]

Step 1: 3,3-difluoropyrrolidine-1-carbonyl chloride

To a solution of 3,3-difluoropyrrolidine (0.2 g, 1.87 mmol, 1 eq) in THE (8 mL) was added DIEA (482.68 mg, 3.73 mmol, 650.51 uL, 2 eq), and then triphosgene (759.18 mg, 2.56 mmol, 1.37 eq) was added at 0° C. The mixture was stirred at 25° C. for 2 h. Upon completion, the combined organic layers were concentrated under reduced pressure to give the crude product 3,3-difluoropyrrolidine-1-carbonyl chloride (260 mg, crude) as yellow oil.

Step 2: (1R,2S,5S)-3-[(2S)-2-amino-3,3-dimethyl-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide

A solution of tert-butyl N-[(1S)-1-[(1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl-propyl]carbamate (0.4 g, 730.35 umol, 1 eq) in HCl/dioxane (4 M, 3 mL, 16.43 eq) was stirred at 25° C. for 1 h. Upon completion, the reaction mixture was concentrated under reduced pressure to give the crude product (1R,2S,5S)-3-[(2S)-2-amino-3,3-dimethyl-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide (280 mg, crude, HCl) as a yellow oil. MS (ESI) m/z 448.1 [M+H]⁺

Step 3: (1R,2S,5S)—N-[(1S)-2-amino-2-oxo-1-[[(6R)-5-oxo-4-azaspiro[2.4]heptan-6-yl]methyl]ethyl]-3-[(2S)-2-[(3,3-difluoropyrrolidine-1-carbonyl)amino]-3,3-dimethyl-butanoyl]-6,6-dimethyl-3-azabicyclo[3.1.0]hexane-2-carboxamide

To a solution of 3,3-difluoropyrrolidine-1-carbonyl chloride (98.08 mg, 578.48 umol, 1 eq) in THE (5 mL) was added DIEA (224.29 mg, 1.74 mmol, 302.28 uL, 3 eq), and then (1R,2S,5S)-3-[(2S)-2-amino-3,3-dimethyl-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide (0.28 g, 578.48 umol, 1 eq, HCl) was added at 0° C. The mixture was stirred at 25° C. for 26 h. Upon completion, the reaction mixture was quenched by addition NaHCO₃(15 mL) and extracted with EA (10 mL*6). The combined organic layers were dried over Na₂SO₄, filtered and concentrated under reduced pressure and was purified by column chromatography (SiO₂, ethyl acetate:dichloromethane=99:1 to 97:1 to 95:1 to 93:1 to 90:1) to give (1R,2S,5S)—N-[(1S)-2-amino-2-oxo-1-[[(6R)-5-oxo-4-azaspiro[2.4]heptan-6-yl]methyl]ethyl]-3-[(2S)-2-[(3,3-difluoropyrrolidine-1-carbonyl)amino]-3,3-dimethyl-butanoyl]-6,6-dimethyl-3-azabicyclo[3.1.0]hexane-2-carboxamide (70 mg, 120.55 umol, 20.84% yield) as a white solid. MS (ESI) m/z 581.4 [M+H]⁺

Step 4: (1R,2S,5S)—N-[(1S)-1-cyano-2-[(6R)-5-oxo-4-azaspiro[2.4]heptan-6-yl]ethyl]-3-[(2S)-2-[(3,3-difluoropyrrolidine-1-carbonyl)amino]-3,3-dimethyl-butanoyl]-6,6-dimethyl-3-azabicyclo[3.1.0]hexane-2-carboxamide

To a solution of (1R,2S,5S)—N-[(1S)-2-amino-2-oxo-1-[[(6R)-5-oxo-4-azaspiro[2.4]heptan-6-yl]methyl]ethyl]-3-[(2S)-2-[(3,3-difluoropyrrolidine-1-carbonyl)amino]-3,3-dimethyl-butanoyl]-6,6-dimethyl-3-azabicyclo[3.1.0]hexane-2-carboxamide (60 mg, 103.33 umol, 1 eq) in DCM (3 mL) was added Burgess reagent (73.87 mg, 309.99 umol, 3 eq). The mixture was stirred at 25° C. for 4 h. Upon completion, the reaction mixture was quenched with water (0.2 mL) and blow-dried with N₂ and was purified by prep-HPLC (column: Waters Xbridge Prep OBD C18 150*40 mm*10 um; mobile phase: [water (NH₄HCO₃)-ACN]; B %: 35%-55%, 8 min) to give (1R,2S,5S)—N-[(1S)-1-cyano-2-[(6R)-5-oxo-4-azaspiro[2.4]heptan-6-yl]ethyl]-3-[(2S)-2-[(3,3-difluoropyrrolidine-1-carbonyl)amino]-3,3-dimethyl-butanoyl]-6,6-dimethyl-3-azabicyclo[3.1.0]hexane-2-carboxamide (15 mg, 26.66 umol, 25.80% yield, 100% purity) as a white solid. MS (ESI) m/z 563.2 [M+H]⁺

¹H NMR (400 MHz, DMSO-d₆) δ=9.00 (d, J=8.6 Hz, 1H), 7.75 (s, 1H), 5.76 (d, J=9.1 Hz, 1H), 4.92 (td, J=5.4, 8.7 Hz, 1H), 4.17 (d, J=9.2 Hz, 1H), 4.13 (s, 1H), 3.83 (br s, 2H), 3.79-3.71 (m, 1H), 3.64-3.50 (m, 3H), 2.69-2.64 (m, 1H), 2.39-2.31 (m, 2H), 2.23-2.14 (m, 1H), 2.01-1.90 (m, 2H), 1.83-1.75 (m, 1H), 1.56-1.50 (m, 1H), 1.27 (d, J=7.6 Hz, 1H), 1.02 (s, 3H), 0.94 (s, 9H), 0.87 (s, 3H), 0.72 (br d, J=9.2 Hz, 1H), 0.58-0.51 (m, 3H)

Example 32: Synthesis of N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl-propyl]-7,7-difluoro-2-azaspiro[3.3]heptane-2-carboxamide (Compound 281)

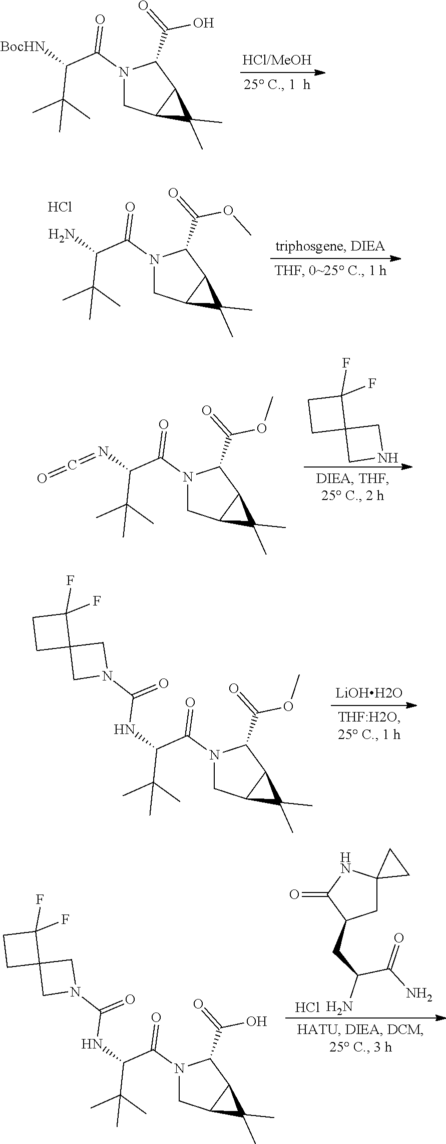

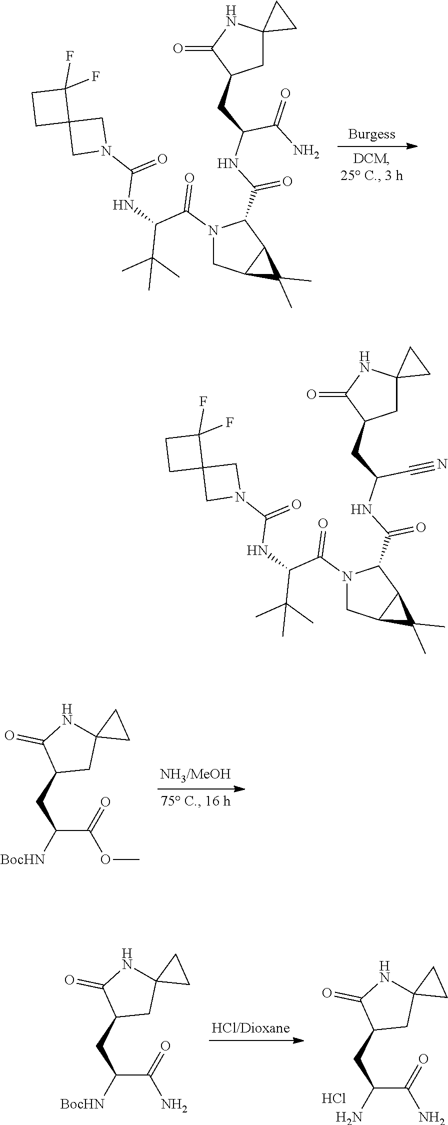 [SMILES: CC(C)(C)[C@H](NC(=O)N1CC2(CCC2(F)F)C1)C(=O)N1C[C@H]2[C@@H]([C@H]1C(=O)N[C@@H](C[C@@H]1CC3(CC3)NC1=O)C(N)=O)C2(C)C ; CC(C)(C)[C@H](NC(=O)N1CC2(CCC2(F)F)C1)C(=O)N1C[C@H]2[C@@H]([C@H]1C(=O)N[C@H](C#N)C[C@@H]1CC3(CC3)NC1=O)C2(C)C ; COC(=O)[C@H](C[C@@H]1CC2(CC2)NC1=O)NC(=O)OC(C)(C)C ; CC(C)(C)OC(=O)N[C@@H](C[C@@H]1CC2(CC2)NC1=O)C(N)=O ; Cl ; NC(=O)[C@@H](N)C[C@@H]1CC2(CC2)NC1=O]

Step 1: N-[(1S)-2-amino-2-oxo-1-[[(6R)-5-oxo-4-azaspiro[2.4]heptan-6-yl]methyl]ethyl]-3,3-dimethyl-butanamide

A mixture of methyl (2S)-2-(tert-butoxycarbonylamino)-3-[(6R)-5-oxo-4-azaspiro[2.4]heptan-6-yl]propanoate (500 mg, 1.60 mmol, 1 eq) in NH₃/MeOH (5 mL) was stirred at 75° C. for 16 h. Upon completion, the reaction mixture was concentrated under reduced pressure to afford N-[(1S)-2-amino-2-oxo-1-[[(6R)-5-oxo-4-azaspiro[2.4]heptan-6-yl]methyl]ethyl]-3,3-dimethyl-butanamide (420 mg, crude) as a white solid.

Step 2: (2S)-2-amino-3-[(6R)-5-oxo-4-azaspiro[2.4]heptan-6-yl]propanamide

A solution of tert-butyl N-[(1S)-2-amino-2-oxo-1-[[(6R)-5-oxo-4-azaspiro[2.4]heptan-6-yl]methyl]ethyl]carbamate (300 mg, 1.01 mmol, 1 eq) in HCl/dioxane (2 mL) was stirred at 25° C. for 1 h. Upon completion, the reaction mixture was concentrated under reduced pressure to afford (2S)-2-amino-3-[(6R)-5-oxo-4-azaspiro[2.4]heptan-6-yl]propanamide (280 mg, crude, HCl) as a white solid.

Step 3: methyl (1R,2S,5S)-3-[(2S)-2-amino-3,3-dimethyl-butanoyl]-6,6-dimethyl-3-azabicyclo[3.1.0]hexane-2-carboxylate

A solution of (1R,2S,5S)-3-[(2S)-2-(tert-butoxycarbonylamino)-3,3-dimethyl-butanoyl]-6,6-dimethyl-3-azabicyclo[3.1.0]hexane-2-carboxylic acid (350 mg, 949.88 umol, 1 eq) in HCl/MeOH (4 mL) was stirred at 25° C. for 1 h. Upon completion, the reaction was concentrated in vacuum to afford methyl (1R,2S,5S)-3-[(2S)-2-amino-3,3-dimethyl-butanoyl]-6,6-dimethyl-3-azabicyclo[3.1.0]hexane-2-carboxylate (340 mg, crude, HCl) as a white solid.

Step 4: methyl (1R,2S,5S)-3-[(2S)-2-isocyanato-3,3-dimethyl-butanoyl]-6,6-dimethyl-3-azabicyclo[3.1.0]hexane-2-carboxylate

To a solution of methyl (1R,2S,5S)-3-[(2S)-2-amino-3,3-dimethyl-butanoyl]-6,6-dimethyl-3-azabicyclo[3.1.0]hexane-2-carboxylate (340 mg, 627.28 umol, 1 eq, HCl) in THE (2 mL) was added DIEA (243.21 mg, 1.88 mmol, 327.78 uL, 3 eq) dropwise at 0° C. After addition, the mixture was stirred at 0° C. temperature for 15 min, and then triphosgene (260.60 mg, 878.19 umol, 1.4 eq) in THE (1 mL) was added dropwise at 0° C. The resulting mixture was stirred at 20° C. for 1.5 h. Upon completion, the reaction mixture was concentrated in vacuum to dryness to afford methyl (1R,2S,5S)-3-[(2S)-2-isocyanato-3,3-dimethyl-butanoyl]-6,6-dimethyl-3-azabicyclo[3.1.0]hexane-2-carboxylate (335 mg, crude) as a yellow solid. MS (ESI) m/z 309.2 [M+H]⁺

Step 5: methyl (1R,2S,5S)-3-[(2S)-2-[(7,7-difluoro-2-azaspiro(3.3]heptane-2-carbonyl)amino]-3,3-dimethyl-butanoyl]-6,6-dimethyl-3-azabicyclo[3.1.0]hexane-2-carboxylate

To a solution of methyl (1R,2S,5S)-3-[(2S)-2-isocyanato-3,3-dimethyl-butanoyl]-6,6-dimethyl-3-azabicyclo[3.1.0]hexane-2-carboxylate (335.00 mg, 583.71 umol, 1 eq) in THE (5 mL) was added 7,7-difluoro-2-azaspiro[3.3]heptane (144.27 mg, 583.71 umol, 1 eq, TFA) and DIEA (150.88 mg, 1.17 mmol, 203.34 uL, 2 eq). The mixture was stirred at 25° C. for 2 h. Upon completion, the reaction mixture was diluted with H₂O 10 mL and extracted with ethyl acetate 20 mL (10 mL*2). The combined organic layers were washed with brine 20 mL (20 mL*1), dried over Na₂SO₄, filtered and concentrated under reduced pressure to give a residue. The residue was purified by column chromatography (SiO₂, Petroleum ether:Ethyl acetate=10:1 to 3:1) to afford methyl (1R,2S,5S)-3-[(2S)-2-[(7,7-difluoro-2-azaspiro[3.3]heptane-2-carbonyl)amino]-3,3-dimethyl-butanoyl]-6,6-dimethyl-3-azabicyclo[3.1.0]hexane-2-carboxylate (280 mg, 339.74 umol).

Step 6: (1R,2S,5S)-3-[(2S)-2-[(7,7-difluoro-2-azaspiro(3.3]heptane-2-carbonyl)amino]-3,3-dimethyl-butanoyl]-6,6-dimethyl-3-azabicyclo[3.1.0]hexane-2-carboxylic acid

To a solution of methyl (1R,2S,5S)-3-[(2S)-2-[(7,7-difluoro-2-azaspiro[3.3]heptane-2-carbonyl)amino]-3,3-dimethyl-butanoyl]-6,6-dimethyl-3-azabicyclo[3.1.0]hexane-2-carboxylate (280 mg, 339.74 umol, 1 eq) in THE (1 mL) and H₂O (1 mL) was added LiOH.H₂O (64.16 mg, 1.53 mmol, 4.5 eq). The mixture was stiffed at 25° C. for 1 h. Upon completion, the mixture was adjust pH to 3-4 by 10% citric acid solution, and extracted with ethyl acetate (10 mL*2). The combined organic layers were washed with brine (20 mL*2), dried over Na₂SO₄, filtered and concentrated under reduced pressure to afford (1R,2S,5S)-3-[(2S)-2-[(7,7-difluoro-2-azaspiro[3.3]heptane-2-carbonyl)amino]-3,3-dimethyl-butanoyl]-6,6-dimethyl-3-azabicyclo[3.1.0]hexane-2-carboxylic acid (220 mg, 327.50 umol) as white solid.

Step 7: N-[(1S)-1-[(1R,2S,5S)-2-[[(1S)-2-amino-2-oxo-1-[[(6R)-5-oxo-4-azaspiro[2.4]heptan-6-yl/methyl/ethyl]carbamoyl]-6,6-dimethyl-3-azabicyclo[3.1.0]hexane-3-carbonyl]-2,2-dimethyl propyl]-7,7-difluoro-2-azaspiro[3.3]heptane-2-carboxamide

A mixture of (1R,2S,5S)-3-[(2S)-2-[(7,7-difluoro-2-azaspiro[3.3]heptane-2-carbonyl)amino]-3,3-dimethyl-butanoyl]-6,6-dimethyl-3-azabicyclo[3.1.0]hexane-2-carboxylic acid (220 mg, 655.00 umol, 1 eq) and (2S)-2-amino-3-[(6R)-5-oxo-4-azaspiro[2.4]heptan-6-yl]propanamide (129.19 mg, 552.80 umol, 8.44e-1 eq, HCl) in DCM (2 mL) was added HATU (498.10 mg, 1.31 mmol, 2 eq) and DIEA (253.96 mg, 1.96 mmol, 342.26 uL, 3 eq). The mixture was stirred at 25° C. for 2 h. Upon completion, the reaction mixture was quenched by addition H₂O 10 mL, and then extracted with DCM (15 mL*3). The combined organic layers were dried over Na₂SO₄, filtered and concentrated under reduced pressure to give a residue. The residue was purified by column chromatography (SiO₂, Petroleum ether:Ethyl acetate=3:1 to Ethyl acetate: Methanol=10:1) to afford N [(1S)-1-[(1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl-propyl]-7,7-difluoro-2-azaspiro[3.3]heptane-2-carboxamide (186 mg, 329.65 umol, 45.81% yield) as a white solid.

Step 8: N-[(1S)-1-[(1R,2S,5S)-2-[[(1S)-1-cyano-2-[(6R)-5-oxo-4-azaspiro[2.4]heptan-6-yl/ethyl]carbamoyl]-6,6-dimethyl-3-azabicyclo[3.1.0/hexane-3-carbonyl]-2,2-dimethyl-propyl]-7,7-difluoro-2-azaspiro[3.3]heptane-2-carboxamide

To a solution of N-[(1S)-1-[(1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl-propyl]-7,7-difluoro-2-azaspiro[3.3]heptane-2-carboxamide (186 mg, 304.93 umol, 1 eq) in DCM (5 mL) was added Burgess reagent (290.66 mg, 1.22 mmol, 4 eq). The mixture was stirred at 25° C. for 3 h. Upon completion, the residue was purified by prep-HPLC (column: Waters Xbridge BEH C18 100*30 mm*10 um; mobile phase: [water(NH₄HCO₃)-ACN]; B %: 45%-65%, 8 min) to afford N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl-propyl]-7,7-difluoro-2-azaspiro[3.3]heptane-2-carboxamide (80 mg, 134.60 umol, 44.14% yield, 99.05% purity) as a white solid.

1H NMR (400 MHz, DMSO-d₆) δ ppm 0.48-0.81 (m, 4H) 0.83-0.86 (m, 3H) 0.92 (s, 9H) 1.01 (s, 3H) 1.22-1.29 (m, 1H) 1.52 (br dd, J=7.09, 4.47 Hz, 1H) 1.79 (ddd, J=13.38, 10.94, 5.01 Hz, 1H) 1.88-2.04 (m, 4H) 2.19 (ddd, J=13.41, 10.85, 4.35 Hz, 1H) 2.38-2.46 (m, 2H) 2.60-2.71 (m, 1H) 3.65-3.85 (m, 4H) 3.93 (d, J=9.06 Hz, 1H) 4.04 4.18 (m, 3H) 4.87-4.98 (m, 1H) 6.21 (d, J=9.30 Hz, 1H) 7.72 (s, 1H) 8.97 (d, J=8.58 Hz, 1H)

Example 33: Synthesis of (1R,2S,5S)-N—((S)-1-cyan-2-((R)-5-oxo-4-azaspiro[2.4]heptan-6-yl)ethyl)-3-((2S,3R)-3-methoxy-2-(2,2,2-trifluoroacetamido)butanoyl)-6,6-dimethyl-3-azabicyclo[3.1.0]hexane-2-carboxamide (Compound 309)

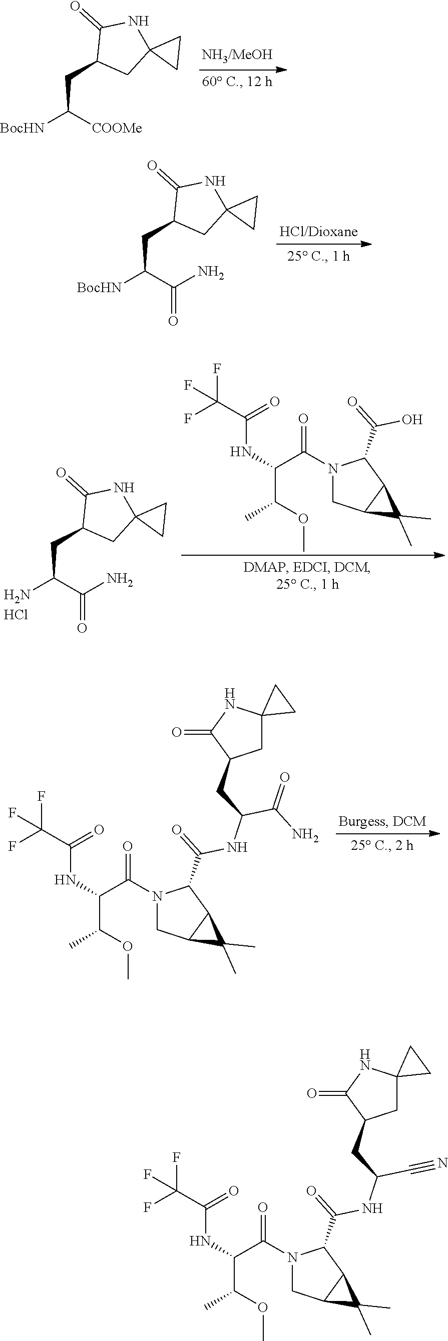

Step 1: tert-butyl ((S)-1-amino-1-oxo-3-((R)-5-oxo-4-azaspiro[2.4]heptan-6-yl)propan-2-yl)carbamate

A solution of methyl (2S)-2-(tert-butoxycarbonylamino)-3-[(6R)-5-oxo-4-azaspiro[2.4]heptan-6-yl]propanoate (500 mg, 1.60 mmol, 1 eq) in NH₃/MeOH (7 M, 10 mL, 43.73 eq) was stirred for 12 h at 60° C. The reaction mixture was concentrated under reduced pressure to remove the solvent and give tert-butyl N-[(1S)-2-amino-2-oxo-1-[[(6R)-5-oxo-4-azaspiro[2.4]heptan-6-yl]methyl]ethyl]carbamate (480 mg, crude) as a white solid.

Step 2: (S)-2-amino-3-((R)-5-oxo-4-azaspiro[2.4]heptan-6-yl)propanamide

A solution of tert-butyl N-[(1S)-2-amino-2-oxo-1-[[(6R)-5-oxo-4-azaspiro[2.4]heptan-6-yl]methyl]ethyl]carbamate (480 mg, 1.61 mmol, 1 eq) in HCl/dioxane (4 M, 5 mL, 12.39 eq) was stirred at 25° C. for 1 h. Upon completion, the resulting solution was concentrated in vacuum to give (2S)-2-amino-3-[(6R)-5-oxo-4-azaspiro[2.4]heptan-6-yl]propanamide (320 mg, crude, HCl) as a white solid.

Step 3: (1R,2S,5S)-N—((S)-1-amino-1-oxo-3-((R)-5-oxo-4-azaspiro[2.4]heptan-6-yl)propan-2-yl)-3-((2S,3R)-3-methoxy-2-(2,2,2-trifluoroacetamido)butanoyl)-6,6-dimethyl-3-azabicyclo[3.1.0]hexane-2-carboxamide

To a solution of (2S)-2-amino-3-[(6R)-5-oxo-4-azaspiro[2.4]heptan-6-yl]propanamide (320 mg, 1.37 mmol, 1 eq, HCl) in DCM (6 mL) was added (1R,2S,5S)-3-[(2S,3R)-3-methoxy-2-[(2,2,2-trifluoroacetyl)amino]butanoyl]-6,6-dimethyl-3-azabicyclo[3.1.0]hexane-2-carboxylic acid (501.62 mg, 1.37 mmol, 1 eq) and DMAP (501.86 mg, 4.11 mmol, 3 eq), then EDCI (525.00 mg, 2.74 mmol, 2 eq) was added, and the mixture was stirred for 1 h at 25° C. Upon completion, the resulting solution was poured into H₂O (20 mL), and then extracted with EtOAc (30 mL*2), the combined organic phase was washed with 7% citric acid (20 mL*2), NaHCO₃(30 mL), and brine (30 mL*3), dried over Na₂SO₄, filtered and concentrated to give a crude product. The residue was purified by column chromatography (SiO₂, Petroleum ether/Ethyl acetate=1/1 to EA/MeOH=5/1) to give (1R,2S,5S)—N-[(1S)-2-amino-2-oxo-1-[[(6R)-5-oxo-4-azaspiro[2.4]heptan-6-yl]methyl]ethyl]-3-[(2S,3R)-3-methoxy-2-[(2,2,2-trifluoroacetyl)amino]butanoyl]-6,6-dimethyl-3-azabicyclo[3.1.0]hexane-2-carboxamide (230 mg, 421.59 umol, 30.79% yield) as a light yellow oil.

Step 4: (1R,2S,5S)-N—((S)-1-cyan-2-((R)-5-oxo-4-azaspiro[2.4]heptan-6-yl)ethyl)-3-((2S,3R)-3-methoxy-2-(2,2,2-trifluoroacetamido)butanoyl)-6,6-dimethyl-3-azabicyclo[3.1.0]hexane-2-carboxamide

To a solution of (1R,2S,5S)—N-[(1S)-2-amino-2-oxo-1-[[(6R)-5-oxo-4-azaspiro[2.4]heptan-6-yl]methyl]ethyl]-3-[(2S,3R)-3-methoxy-2-[(2,2,2-trifluoroacetyl)amino]butanoyl]-6,6-dimethyl-3-azabicyclo[3.1.0]hexane-2-carboxamide (230 mg, 421.59 umol, 1 eq) in DCM (3 mL) was added methoxycarbonyl-(triethylammonio)sulfonyl-azanide (200.94 mg, 843.19 umol, 2 eq), and then the mixture was stirred for 2 h at 25° C. Upon completion, the resulting solution was quenched with H₂O (0.2 mL), then was concentrated in vacuum, the residue was purified by prep-HPLC (column: Waters Xbridge Prep OBD C18 150*40 mm*10 um; mobile phase: [water(NH₄HCO₃)-ACN]; B %: 25%-50%, 8 min) to give (1R,2S,5S)—N-[(1S)-1-cyano-2-[(6R)-5-oxo-4-azaspiro[2.4]heptan-6-yl]ethyl]-3-[(2S,3R)-3-methoxy-2-[(2,2,2-trifluoroacetyl)amino]butanoyl]-6,6-dimethyl-3-azabicyclo[3.1.0]hexane-2-carboxamide (52.27 mg, 99.08 umol, 23.50% yield, 100% purity) as a white solid. MS (ESI) m/z 528.2[M+H]⁺.

1H NMR (400 MHz, DMSO-d₆) δ=9.99-9.83 (m, 1H), 9.03 (br d, J=8.6 Hz, 1H), 7.75 (s, 1H), 4.99-4.88 (m, 1H), 4.41-4.28 (m, 1H), 4.17-4.10 (m, 1H), 3.97-3.78 (m, 2H), 3.67-3.56 (m, 1H), 3.24 (s, 3H), 2.71-2.60 (m, 1H), 2.26-2.11 (m, 1H), 2.04 1.76 (m, 3H), 1.64-1.52 (m, 1H), 1.37-1.28 (m, 1H), 1.18-1.10 (m, 3H), 1.04-1.00 (m, 3H), 0.89-0.83 (m, 3H), 0.79-0.73 (m, 1H), 0.66-0.48 (m, 3H).

Example 34: Synthesis of (1R,2S,5S)—N-[(1S)-1-cyano-2-[(6R)-5-oxo-4-azaspiro[2.4]heptan-6-yl]ethyl]-3-[(2S)-2-[[3-(2-fluoroethyl)pyrrolidine-1-carbonyl]amino]-3,3-dimethyl-butanoyl]-6,6-dimethyl-3-azabicyclo[3.1.0]hexane-2-carboxamide (Compound 282)

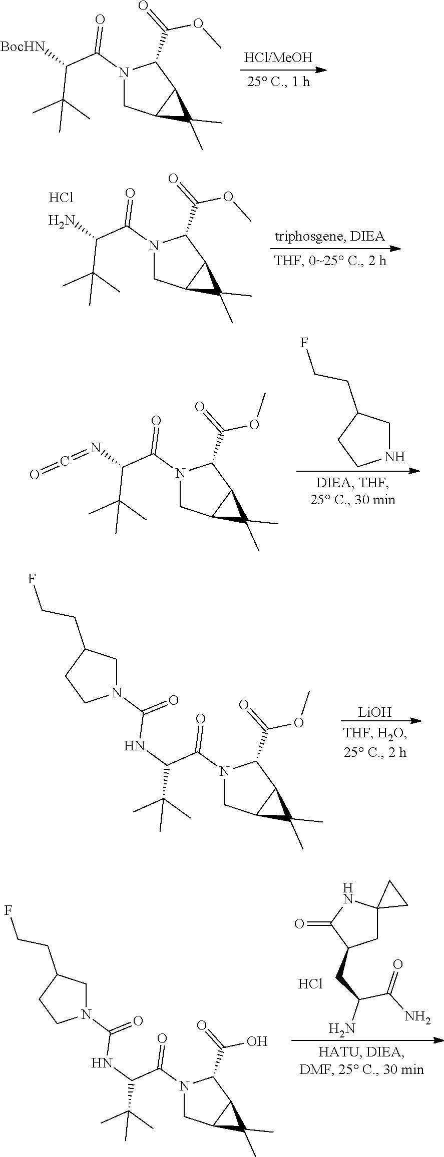

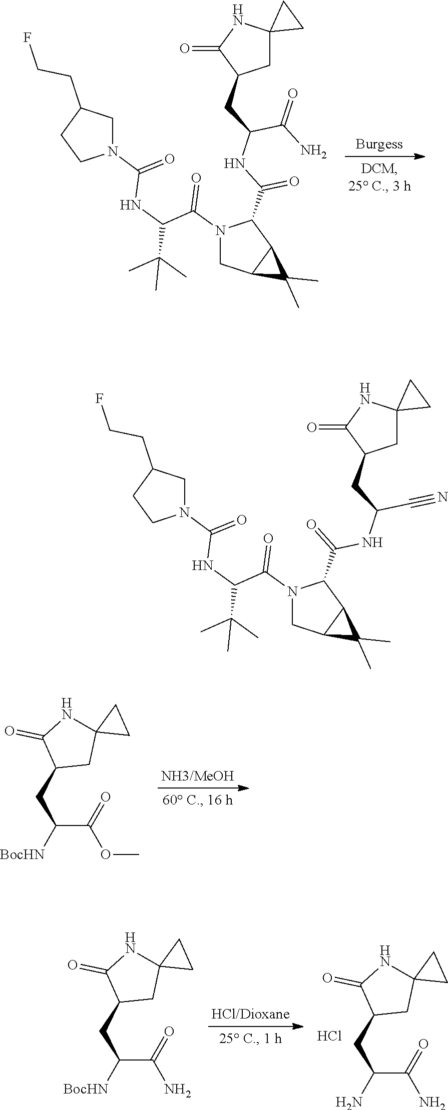

Step 1: tert-butyl N-[(1S)-2-amino-2-oxo-1-[[(6R)-5-oxo-4-azaspiro[2.4]heptan-6-yl]methyl]ethyl]carbamate

A solution of methyl (2S)-2-(tert-butoxycarbonylamino)-3-[(6R)-5-oxo-4-azaspiro[2.4]heptan-6-yl]propanoate (500 mg, 1.60 mmol, 1 eq) in NH₃/MeOH (7 M, 28.57 mL, 124.94 eq) was stirred at 60° C. for 16 h. Upon completion, the reaction mixture was concentrated under reduced pressure to give tert-butyl N-[(1S)-2-amino-2-oxo-1-[[(6R)-5-oxo-4-azaspiro[2.4]heptan-6-yl]methyl]ethyl]carbamate (450 mg, 1.41 mmol, 87.93% yield, 93% purity) as a white solid. MS (ESI) m/z 298.2 [M+H]⁺.

Step 2: (2S)-2-amino-3-[(6R)-5-oxo-4-azaspiro[2.4]heptan-6-yl]propanamide

A mixture of tert-butyl N-[(1S)-2-amino-2-oxo-1-[[(6R)-5-oxo-4-azaspiro[2.4]heptan-6-yl]methyl]ethyl]carbamate (300 mg, 1.01 mmol, 1 eq) was added HCl/dioxane (4 M, 10 mL, 39.65 eq). The mixture was stirred at 25° C. for 1 h. Upon completion, the reaction mixture was concentrated under reduced pressure to give (2S)-2-amino-3-[(6R)-5-oxo-4-azaspiro[2.4]heptan-6-yl]propanamide (210 mg, 898.61 umol, 89.07% yield, HCl) as a white solid. MS (ESI) m/z 198.2 [M+H]⁺.

Step 3: methyl (1R,2S,5S)-3-[(2S)-2-amino-3,3-dimethyl-butanoyl]-6,6-dimethyl-3-azabicyclo[3.1.01 hexane-2-carboxylate

A mixture of methyl (1R,2S,5S)-3-[(2S)-2-(tert-butoxycarbonylamino)-3,3-dimethyl-butanoyl]-6,6-dimethyl-3-azabicyclo[3.1.0]hexane-2-carboxylate (800.00 mg, 2.09 mmol, 1 eq) was added with HCl/MeOH (4 M, 10 mL, 19.12 eq). The mixture was stirred at 25° C. for 1 h. Upon completion, the reaction mixture was concentrated under reduced pressure to give methyl (1R,2S,5S)-3-[(2S)-2-amino-3,3-dimethyl-butanoyl]-6,6-dimethyl-3-azabicyclo[3.1.0]hexane-2-carboxylate (605 mg, 1.90 mmol, 90.72% yield, HCl) as a white solid.

Step 4: methyl (1R,2S,5S)-3-[(2S)-2-isocyanato-3,3-dimethyl-butanoyl]-6,6-dimethyl-3-azabicyclo 13.1.0]hexane-2-carboxylate

To a solution of methyl (1R,2S,5S)-3-[(2S)-2-amino-3,3-dimethyl-butanoyl]-6,6-dimethyl-3-azabicyclo[3.1.0]hexane-2-carboxylate (550 mg, 1.73 mmol, 1 eq, HCl) in THE (10 mL) was added DIEA (668.84 mg, 5.18 mmol, 901.40 uL, 3 eq), and then bis(trichloromethyl) carbonate (740 mg, 2.49 mmol, 1.45 eq) was added at 0° C. The mixture was stirred at 25° C. for 2 h. Upon completion, the mixture was concentrated under reduced pressure to give methyl (1R,2S,5S)-3-[(2S)-2-isocyanato-3,3-dimethyl-butanoyl]-6,6-dimethyl-3-azabicyclo[3.1.0]hexane-2-carboxylate (500 mg, 843.14 umol, 48.88% yield, 52% purity) as a yellow solid. MS (ESI) m/z 309.2 [M+H]⁺.

Step 5: methyl (1R,2S,5S)-3-[(2S)-2-[[3-(2 fluoroethyl)pyrrolidine-1-carbonyl]amino]-3,3-dimethyl-butanoyl]-6,6-dimethyl-3-azabicyclo[3.1.0]hexane-2-carboxylate

To a solution of methyl (1R,2S,5S)-3-[(2S)-2-isocyanato-3,3-dimethyl-butanoyl]-6,6-dimethyl-3-azabicyclo[3.1.0]hexane-2-carboxylate (500 mg, 1.62 mmol, 1 eq) and 3-(2-fluoroethyl)pyrrolidine (189.79 mg, 1.62 mmol, 1 eq) in THE (5 mL) was added DIEA (418.13 mg, 3.23 mmol, 563.52 uL, 2 eq) at 25° C. The mixture was stirred at 25° C. for 30 min. Upon completion, the reaction mixture was diluted with H₂O 10 mL and extracted with EA 20 mL (10 mL*2). The combined organic layers were washed with brine 20 mL (20 mL*1), dried over Na₂SO₄, filtered and concentrated under reduced pressure to give a residue. The residue was purified by column chromatography (SiO₂, Petroleum ether:Ethyl acetate=10:1 to 3:1). Compound methyl (1R,2S,5S)-3-[(2S)-2-[[3-(2-fluoroethyl)pyrrolidine-1-carbonyl]amino]-3,3-dimethyl-butanoyl]-6,6-dimethyl-3-azabicyclo[3.1.0]hexane-2-carboxylate (338 mg, 659.26 umol, 48.98% yield, 83% purity) was obtained as a white solid. MS (ESI) m/z 426.3 [M+H]⁺.

Step 6: (1R,2S,5S)-3-[(2S)-2-[[3-(2 fluoroethyl)pyrrolidine-1-carbonyl]amino]-3,3-dimethyl-butanoyl]-6,6-dimethyl-3-azabicyclo[3.1.0]hexane-2-carboxylic acid

To a solution of methyl (1R,2S,5S)-3-[(2S)-2-[[3-(2-fluoroethyl)pyrrolidine-1-carbonyl]amino]-3,3-dimethyl-butanoyl]-6,6-dimethyl-3-azabicyclo[3.1.0]hexane-2-carboxylate (300 mg, 704.99 umol, 1 eq) in THE (5 mL) and H₂O (5 mL) was added LiOH.H₂O (103.54 mg, 2.47 mmol, 3.5 eq). The mixture was stirred at 25° C. for 2 h. Upon completion, the reaction mixture was adjusted pH=3 by HCl (10%), and then diluted with H₂O 20 mL and extracted with DCM 40 mL (20 mL*2). The combined organic layers were washed with brine 20 mL (20 mL*1), dried over Na₂SO₄, filtered and concentrated under reduced pressure to give (1R,2S,5S)-3-[(2S)-2-[[3-(2-fluoroethyl)pyrrolidine-1-carbonyl]amino]-3,3-dimethyl-butanoyl]-6,6-dimethyl-3-azabicyclo[3.1.0]hexane-2-carboxylic acid (254 mg, 574.03 umol, 81.42% yield, 93% purity) as a white solid. MS (ESI) m/z 410.3 [M−H]⁻.

Step 7: (1R,2S,5S)—N-[(1S)-2-amino-2-oxo-1-[[(6R)-5-oxo-4-azaspiro[2.4]heptan-6-yl]methyl]ethyl]-3-[(2S)-2-[[3-(2 fluoroethyl)pyrrolidine-1-carbonyl]amino]-3,3-dimethyl-butanoyl]-6,6-dimethyl-3-azabicyclo[3.1.0]hexane-2-carboxamide

To a mixture of (1R,2S,5S)-3-[(2S)-2-[[3-(2-fluoroethyl)pyrrolidine-1-carbonyl]amino]-3,3-dimethyl-butanoyl]-6,6-dimethyl-3-azabicyclo[3.1.0]hexane-2-carboxylic acid (220 mg, 534.62 umol, 1 eq) and (2S)-2-amino-3-[(6R)-5-oxo-4-azaspiro[2.4]heptan-6-yl]propanamide (149.92 mg, 641.54 umol, 1.2 eq, HCl) in DMF (10 mL) was added DIEA (207.28 mg, 1.60 mmol, 279.36 uL, 3 eq), then HATU (406.55 mg, 1.07 mmol, 2 eq) was added. The mixture was stirred at 25° C. for 30 min. Upon completion, the reaction mixture was diluted with H₂O 10 mL and extracted with EA 20 mL (10 mL*2). The combined organic layers were washed with brine 20 mL (20 mL*1), dried over Na₂SO₄, filtered and concentrated under reduced pressure to give a residue. The residue was purified by column chromatography (SiO₂, Petroleum ether:Ethyl acetate=3:1 to Ethyl acetate:MeOH=15:1). Compound (1R,2S,5S)—N-[(1S)-2-amino-2-oxo-1-[[(6R)-5-oxo-4-azaspiro[2.4]heptan-6-yl]methyl]ethyl]-3-[(2S)-2-[[3-(2-fluoroethyl)pyrrolidine-1-carbonyl]amino]-3,3-dimethyl-butanoyl]-6,6-dimethyl-3-azabicyclo[3.1.0]hexane-2-carboxamide (260 mg, 409.33 umol, 76.56% yield, 93% purity) was obtained as a colourless oil. MS (ESI) m/z 591.4 [M+H]⁺.

Step 8: (1R,2S,5S)—N-[(1S)-1-cyano-2-[(6R)-5-oxo-4-azaspiro[2.4]heptan-6-yl]ethyl]-3-[(2S)-2-[[3-(2 fluoroethyl)pyrrolidine-1-carbonyl]aminol-3,3-dimethyl-butanoyl]-6,6-dimethyl-3-azabicyclo[3.1.01 hexane-2-carboxamide

To a mixture of (1R,2S,5S)—N-[(1S)-2-amino-2-oxo-1-[[(6R)-5-oxo-4-azaspiro[2.4]heptan-6-yl]methyl]ethyl]-3-[(2S)-2-[[3-(2-fluoroethyl)pyrrolidine-1-carbonyl]amino]-3,3-dimethyl-butanoyl]-6,6-dimethyl-3-azabicyclo[3.1.0]hexane-2-carboxamide (250 mg, 338.57 umol, 1 eq) in DCM (5 mL) was added Burgess reagent (242.05 mg, 1.02 mmol, 3 eq). The mixture was stirred at 25° C. for 3 h. Upon completion, the reaction mixture was quenched by addition H₂O 20 mL, and then extracted with DCM 40 mL (20 mL*2). The combined organic layers were washed with brine 40 mL (40 mL*1), dried over Na₂SO₄, filtered and concentrated under reduced pressure to give a residue. The residue was purified by neutral prep-HPLC(column: Phenomenex C18 75*30 mm*3 um; mobile phase: [water (NH₄HCO₃)-ACN]; B %:30%-50%, 8 min). Compound (1R,2S,5S)—N-[(1S)-1-cyano-2-[(6R)-5-oxo-4-azaspiro[2.4]heptan-6-yl]ethyl]-3-[(2S)-2-[[3-(2-fluoroethyl)pyrrolidine-1-carbonyl]amino]-3,3-dimethyl-butanoyl]-6,6-dimethyl-3-azabicyclo[3.1.0]hexane-2-carboxamide (105 mg, 183.34 umol, 43.32% yield, 100% purity) was obtained as a white solid. MS (ESI) m/z 573.3 [M+H]⁺.

¹H NMR (400 MHz, DMSO-d₆) δ=8.98 (d, J=8.5 Hz, 1H), 7.74 (s, 1H), 5.18 (t, J=9.0 Hz, 1H), 4.98-4.87 (m, 1H), 4.53 (t, J=6.0 Hz, 1H), 4.42 (t, J=6.0 Hz, 1H), 4.17 (dd, J=3.1, 9.4 Hz, 2H), 4.12 (s, 2H), 3.87-3.78 (m, 2H), 3.24-3.04 (m, 1H), 2.83 (td, J=9.5, 19.0 Hz, 1H), 2.70-2.58 (m, 1H), 2.26-2.11 (m, 2H), 2.02-1.88 (m, 3H), 1.84-1.63 (m, 3H), 1.56-1.43 (m, 2H), 1.32-1.22 (m, 1H), 1.02 (s, 3H), 0.94 (s, 9H), 0.86 (s, 3H), 0.72 (br d, J=9.6 Hz, 1H), 0.61-0.48 (m, 3H).

Example 35: Synthesis of (1RSS)-3-[(2S,3R)-3-tert-butoxy-2-[(2,2,2-trifluoroacetyl)amino]butanoyl]-N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (Compound 285)

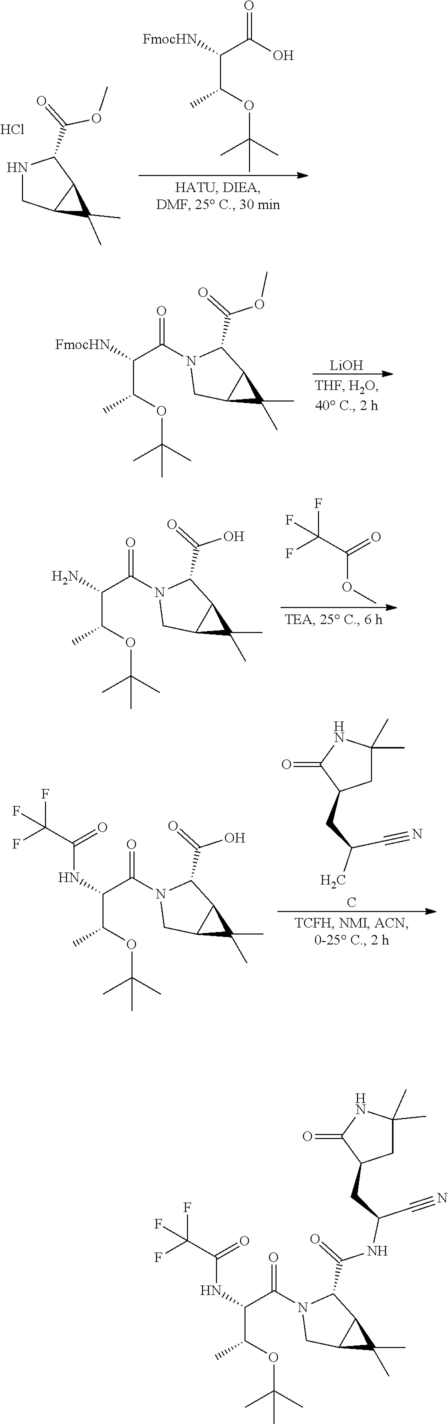

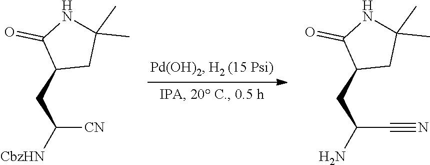 [SMILES: CC1(C)C[C@@H](C[C@@H](C#N)NC(=O)OCc2ccccc2)C(=O)N1 ; CC1(C)C[C@@H](C[C@H](N)C#N)C(=O)N1]

Step 1: (2S)-2-amino-3-[(3R)-5,5-dimethyl-2-oxo pyrrolidin-3-yl]propanenitrile

To a solution of benzyl N-[(1S)-1-cyano-2-[(3R)-5,5-dimethyl-2-oxo-pyrrolidin-3-yl]ethyl]carbamate (700 mg, 2.22 mmol, 1 eq) in isopropanol (10 mL) was added Pd(OH)₂ (700.00 mg, 996.87 umol, 20% purity, 4.49e-1 eq) under N₂. The suspension was degassed and purged with H₂ (4.48 mg, 2.22 mmol, 1 eq) for 3 times. The mixture was stirred under H₂ (15 psi) at 20° C. for 0.5 h. Upon completion, the reaction mixture was filtered and concentrated under reduced pressure to give (2S)-2-amino-3-[(3R)-5,5-dimethyl-2-oxo-pyrrolidin-3-yl]propanenitrile (400 mg, crude) as a yellow oil.

Step 2: methyl (1R,2S,5S)-3-[(3R)-3-tert-butoxy-2-(9H fluoren-9-ylmethoxycarbonylamino)butanoyl]-6,6-dimethyl-3-azabicyclo[3.1.0]hexane-2-carboxylate

A mixture of methyl (1R,2S,5S)-6,6-dimethyl-3-azabicyclo[3.1.0]hexane-2-carboxylate; hydrochloride (2 g, 8.26 mmol, 1 eq, HCl) and (3R)-3-tert-butoxy-2-(9H-fluoren-9-ylmethoxycarbonylamino)butanoic acid (3.28 g, 8.26 mmol, 1 eq) in DMF (20 mL) was added DIEA (3.20 g, 24.78 mmol, 4.32 mL, 3 eq), and then HATU (6.28 g, 16.52 mmol, 2 eq) was added. The mixture was stirred at 25° C. for 30 min. Upon completion, the reaction mixture was diluted with H₂O (40 mL) and extracted with EA (40 mL*3). The combined organic layers were washed with brine (60 mL*1), dried over Na₂SO₄, filtered and concentrated under reduced pressure to give a residue. The residue was purified by column chromatography (SiO₂, Petroleum ether/Ethyl acetate=15/1 to 8/1) to afford methyl (1R,2S,5S)-3-[(3R)-3-tert-butoxy-2-(9H-fluoren-9-ylmethoxycarbonylamino)butanoyl]-6,6-dimethyl-3-azabicyclo[3.1.0]hexane-2-carboxylate (4.3 g, 7.52 mmol, 91.09% yield, 96% purity) as a colorless oil. MS (ESI) m/z 549.3 [M+H]⁺.

Step 3: (1R,2S,5S)-3-[(2S,3R)-2-amino-3-tert-butoxy-butanoyl]-6,6-dimethyl-3-azabicyclo[3.1.0]hexane-2-carboxylic acid

A solution of methyl (1R,2S,5S)-3-[(3R)-3-tert-butoxy-2-(9H-fluoren-9-ylmethoxycarbonylamino)butanoyl]-6,6-dimethyl-3-azabicyclo[3.1.0]hexane-2-carboxylate (4.3 g, 7.84 mmol, 1 eq) and LiOH.H₂O (1.15 g, 27.43 mmol, 3.5 eq) in THE (15 mL) and H₂O (5 mL) was stirred at 40° C. for 2 hours. Upon completion, the reaction mixture was concentrated under reduced pressure to give (1R,2S,5S)-3-[(2S,3R)-2-amino-3-tert-butoxy-butanoyl]-6,6-dimethyl-3-azabicyclo[3.1.0]hexane-2-carboxylic acid (3 g, crude) as a white solid. MS (ESI) m/z 311.0 [M−H]⁺.

Step 4: (1R,2S,5S)-3-[(2S,3R)-3-tert-butoxy-2-[(2,2,2-trifluoroacetyl)amino]butanoyl]-6,6-dimethyl-3-azabicyclo[3.1.0]hexane-2-carboxylic acid

A solution of (1R,2S,5S)-3-[(2S,3R)-2-amino-3-tert-butoxy-butanoyl]-6,6-dimethyl-3-azabicyclo[3.1.0]hexane-2-carboxylic acid (3 g, 9.60 mmol, 1 eq) in TEA (8.75 g, 86.43 mmol, 12.03 mL, 9 eq) was added methyl 2,2,2-trifluoroacetate (12.30 g, 96.03 mmol, 9.68 mL, 10 eq) and stirred at 25° C. for 6 h. Upon completion, the reaction mixture was concentrated in vacuum to remove most of methyl 2,2,2-trifluoroacetate and afford a residue. The combined residue was diluted with EtOAc (20 mL) and washed with 1 M aqueous HCl (20 mL). The organic layer was separated and the aqueous layer was extracted with EtOAc (20 ml*2). The combined organic layer was dried over Na₂SO₄, filtered and concentrated under reduced pressure to give a residue. The residue was triturated with PE: EA=20:1 (20 mL) at 25° C. for 15 min, and then filtered, and washed with PE:EA (20:1)(200 mL) to give the desired product(2 g, 90% purity), then the product (1 g) was purified by prep-HPLC (column: Agela DuraShell C18 250*70 mm*10 um; mobile phase: [water (NH₄HCO₃)⁻ACN]; B %: 1%-35%) to give (1R,2S,5S)-3-[(2S,3R)-3-tert-butoxy-2-[(2,2,2-trifluoroacetyl)amino]butanoyl]-6,6-dimethyl-3-azabicyclo[3.1.0]hexane-2-carboxylic acid (100 mg, 242.40 umol, 2.52% yield, 99% purity) as a white solid.

Step 5: (1R,2S,5S)-3-[(2S,3R)-3-tert-butoxy-2-[(2,2,2-trifluoroacetyl)amino]butanoyl]-N-[(1S)-1-cyano-2-[(3R)-5,5-dimethyl-2-oxo pyrrolidin-3-yl]ethyl]-6,6-dimethyl-3-azabicyclo[3.1.0]hexane-2-carboxamide

To a solution of (1R,2S,5S)-3-[(2S,3R)-3-tert-butoxy-2-[(2,2,2-trifluoroacetyl)amino]butanoyl]-6,6-dimethyl-3-azabicyclo[3.1.0]hexane-2-carboxylic acid (675.75 mg, 1.65 mmol, 1 eq) and (2S)-2-amino-3-[(3R)-5,5-dimethyl-2-oxo-pyrrolidin-3-yl]propanenitrile (300 mg, 1.66 mmol, 1 eq) in acetonitrile (10 mL) was added 1-methylimidazole (271.80 mg, 3.31 mmol, 263.89 uL, 2 eq), then TCFH (928.90 mg, 3.31 mmol, 2 eq) was added at 0° C., the mixture was stirred at 0° C. for 2 h. Upon completion, the reaction mixture was diluted with water (40 mL) and extracted with DCM (15 mL*3). The combined organic layers were dried over Na₂SO₄, filtered and concentrated under reduced pressure to give a residue. The residue was purified by prep-HPLC (column: Waters Xbridge BEH C18 100*30 mm*10 um; mobile phase: [water (NH₄HCO₃)-ACN]; B %: 30%-70%, 10 min) to give (1R,2S,5S)-3-[(2S,3R)-3-tert-butoxy-2-[(2,2,2-trifluoroacetyl)amino]butanoyl]-N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (30 mg, 51.80 umol, 3.13% yield, 98.7% purity) as a white solid. MS (ESI) m/z 571.3 [M+H]⁺.

¹H NMR (400 MHz, DMSO-d₆) δ=9.70 (brd, J=8.0 Hz, 1H), 8.98 (brd, J=8.5 Hz, 1H), 7.89-7.76 (m, 1H), 4.98-4.69 (m, 1H), 4.39 (br t, J=8.2 Hz, 1H), 4.11 (s, 1H), 3.97 (dd, J=5.5, 10.4 Hz, 1H), 3.86 (br dd, J=6.3, 8.2 Hz, 1H), 3.72-3.62 (m, 1H), 2.59 (br dd, J=3.8, 9.1 Hz, 1H),2.20-2.11 (m, 1H), 1.98 (dd, J=8.6, 12.3 Hz, 1H), 1.79 1.67 (m, 1H), 1.61-1.43 (m, 2H), 1.33 (d, J=7.5 Hz, 1H), 1.08 (br s, 18H), 1.03-0.82 (m, 6H).

Example 36: Synthesis of (1R,2S,5S)—N-[(1S)-1-cyano-2-[(6R)-5-oxo-4-azaspiro[2.4]heptan-6-yl]ethyl]-3-[(2S)-2-[(1-ethyl-5,5-difluoro-piperidine-3-carbonyl)amino]-3,3-dimethyl-butanoyl]-6,6-dimethyl-3-azabicyclo[3.1.0]hexane-2-carboxamide (Compound 286)

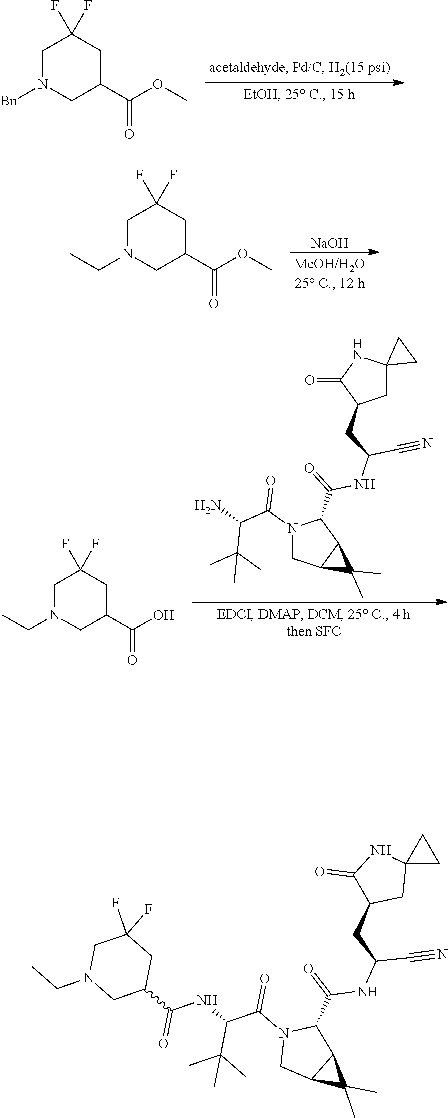

Step 1: methyl 1-ethyl-5,5-difluoro piperidine-3-carboxylate

A mixture of Pd/C (400 mg, 400.00 mmol, 10% purity, 269.29 eq) in EtOH (5 mL) was added methyl 1-benzyl-5,5-difluoro-piperidine-3-carboxylate (400 mg, 1.49 mmol, 1 eq) and acetaldehyde (5 M, 594.16 uL, 40% purity, 2 eq) then the suspension was degassed under vacuum and purged with H₂ several times. The mixture was stirred under H₂ (353.25 ug, 175.24 umol) (15 psi) at 25° C. for 15 h. Upon completion, the reaction mixture was filtered and concentrated under reduced pressure to give a residue, the residue was purified by silica gel column chromatography (SiO₂, PE:EA=4:1) to give methyl 1-ethyl-5,5-difluoro-piperidine-3-carboxylate (233 mg, 1.12 mmol, 75.70% yield) as a white oil.

Step 2: methyl (2S)-2-[[2-[(6,7-difluoro-1 H-indole-2-carbonyl)amino]-3-(1-fluorocyclopropyl)propanoyl]amino]-3-[(6R)-5-oxo-4-azaspiro[2.4]heptan-6-yl]propanoate

A solution of methyl 1-ethyl-5,5-difluoro-piperidine-3-carboxylate (200 mg, 965.17 umol, 1 eq) and NaOH (38.61 mg, 965.17 umol, 1 eq) in MeOH (3 mL) and then H₂O (3 mL) was stirred at 25° C. for 12 h. Upon completion, the residue was concentrated in vacuum, and washed with DCM (10 mL*2) and concentrated to give 1-ethyl-5,5-difluoro-piperidine-3-carboxylic acid (170 mg, 879.96 umol, 91.17% yield) as a white solid.

Step 3: (1R,2S,5S)—N-[(1S)-1-cyano-2-[(6R)-5-oxo-4-azaspiro[2.4]heptan-6-yl]ethyl]-3-[(2S)-2-[(1-ethyl-5,5-difluoro piperidine-3-carbonyl)amino]-3,3-dimethyl-butanoyl]-6,6-dimethyl-3-azabicyclo[3.1.0]hexane-2-carboxamide

To a solution of (1R,2S,5S)-3-[(2S)-2-amino-3,3-dimethyl-butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (300 mg, 698.40 umol, 7.94e-1 eq) and 1-ethyl-5,5-difluoro-piperidine-3-carboxylic acid (170 mg, 879.96 umol, 1 eq) in DCM (8 mL) was added DMAP (322.51 mg, 2.64 mmol, 3 eq) and EDCI (337.38 mg, 1.76 mmol, 2 eq). The resulting mixture was stirred at 25° C. for 2 h. Upon completion, the reaction mixture was diluted with H₂O 10 mL and then extracted with ethyl acetate (10 mL*2). The combined organic layers were washed with 15% citric acid (10 mL*2), NaHCO₃(10 mL) and brine (10 mL) dried over Na₂SO₄, filtered and concentrated under reduced pressure to give a residue. The residue was purified by prep-HPLC column: Waters Xbridge Prep OBD C18 150*40 mm*10 um; mobile phase: [water (NH₄HCO₃)-ACN]; B %: 35%-55%, 8 min, which was further separated by SFC column: DAICEL CHIRALPAK AD (250 mm*30 mm, 10 um); mobile phase: [0.1% NH₃H₂O IPA]; B %: 23%-23%, 7 min) to give (1R,2S,5S)—N-[(1S)-1-cyano-2-[(6R)-5-oxo-4-azaspiro[2.4]heptan-6-yl]ethyl]-3-[(2S)-2-[(1-ethyl-5,5-difluoro-piperidine-3-carbonyl)amino]-3,3-dimethyl-butanoyl]-6,6-dimethyl-3-azabicyclo[3.1.0]hexane-2-carboxamide Isomer 1 (47.35 mg, 78.30 umol, 8.90% yield, 100% purity) as a white solid. MS (ESI) m/z 605.4 [M+H]⁺.

¹H NMR (400 MHz, DMSO-d₆) δ=8.97 (d, J=8.5 Hz, 1H), 8.18 (d, J=8.7 Hz, 1H), 7.74 (s, 1H), 4.92 (ddd, J=5.3, 8.4, 10.6 Hz, 1H), 4.33 (d, J=8.7 Hz, 1H), 4.13 (s, 1H), 3.88-3.81 (m, 1H), 3.79-3.73 (m, 1H), 3.03-2.92 (m, 1H), 2.91-2.82 (m, 1H), 2.75-2.60 (m, 2H), 2.45-2.39 (m, 2H), 2.36-2.13 (m, 2H), 2.07-1.76 (m, 6H), 1.52 (dd, J=5.4, 7.4 Hz, 1H), 1.27 (d, J=7.6 Hz, 1H), 1.02 (s, 3H), 0.99-0.95 (m, 3H), 0.92 (s, 9H), 0.83 (s, 3H), 0.77-0.70 (m, 1H), 0.59-0.50 (m, 3H).

(1R,2S,5S)—N-[(1S)-1-Cyano-2-[(6R)-5-oxo-4-azaspiro[2.4]heptan-6-yl]ethyl]-3-[(2S)-2-[(1-ethyl-5,5-difluoro-piperidine-3-carbonyl)amino]-3,3-dimethyl-butanoyl]-6,6-dimethyl-3-azabicyclo[3.1.0]hexane-2-carboxamide (48.70 mg, 80.53 umol, 9.15% yield, 100% purity) was obtained as a white solid. MS (ESI) m/z 605.4 [M+H]⁺.

¹H NMR (400 MHz, DMSO-d₆) δ=8.98 (d, J=8.5 Hz, 1H), 8.18 (d, J=8.7 Hz, 1H), 7.74 (s, 1H), 4.92 (ddd, J=5.3, 8.5, 10.5 Hz, 1H), 4.31 (d, J=8.7 Hz, 1H), 4.13 (s, 1H), 3.96-3.70 (m, 2H), 2.98 (br t, J=9.9 Hz, 1H), 2.90-2.77 (m, 2H), 2.71-2.58 (m, 1H), 2.46-2.38 (m, 2H), 2.35-2.25 (m, 2H), 2.22-2.20 (m, 1H) 1.91-2.10 (m, 5H), 1.52 (dd, J=5.2, 7.4 Hz, 1H), 1.27 (d, J=7.6 Hz, 1H), 1.02 (s, 3H), 0.98 (t, J=7.2 Hz, 3H), 0.93 (s, 9H), 0.83 (s, 3H), 0.76-0.70 (m, 1H), 0.60-0.48 (m, 3H).

Example 37: Synthesis of (1R,2S,5S)-3-[(2S)-2-[[2-(azetidin-1-yl)-2-methyl-propanoyl]amino]-3,3-dimethyl-butanoyl]-N-[(1S)-1-cyan-2-[(6R)-5-oxo-4-azaspiro[2.4]heptan-6-yl]ethyl]-6,6-dimethyl-3-azabicyclo[3.1.0]hexane-2-carboxamide (Compound 287)

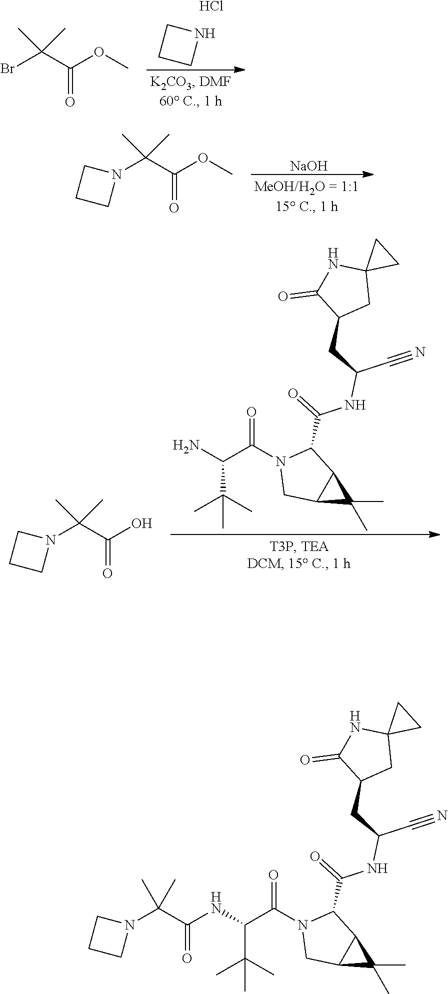

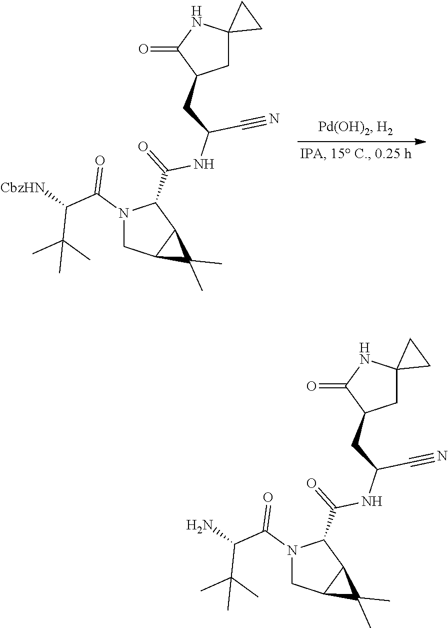 [SMILES: CC(C)(C)[C@H](NC(=O)OCc1ccccc1)C(=O)N1C[C@H]2[C@@H]([C@H]1C(=O)N[C@H](C#N)C[C@@H]1CC3(CC3)NC1=O)C2(C)C ; CC(C)(C)[C@H](N)C(=O)N1C[C@H]2[C@@H]([C@H]1C(=O)N[C@H](C#N)C[C@@H]1CC3(CC3)NC1=O)C2(C)C]

Step 1: (1R,2S,5S)-3-[(2S)-2-amino-3,3-dimethyl-butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide

To a solution of benzyl N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl-propyl]carbamate (300 mg, 532.21 umol, 1 eq) in isopropanol (5 mL) was added Pd(OH)₂/C (20%, 100 mg) under N₂ atmosphere. The suspension was degassed and purged with H₂ for 3 times. The mixture was stirred under H₂ (15 Psi) at 15° C. for 15 min. Upon completion, the reaction solution was diluted by ethyl acetate, filtered through filter paper, and concentrated under reduced pressure to give (1R,2S,5S)-3-[(2S)-2-amino-3,3-dimethyl-butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (220 mg, 512.16 umol, 96.23% yield) as a colorless oil. MS (ESI) m/z 430.5 [M+H]⁺.

Step 2: methyl 2-(azetidin-1-yl)-2-methyl propanoate

To a solution of methyl 2-bromo-2-methyl-propanoate (2 g, 11.05 mmol, 1.43 mL, 1 eq) and azetidine (1.24 g, 13.26 mmol, 1.47 mL, 1.2 eq, HCl) in DMF (20 mL) was added K₂CO₃ (4.58 g, 33.14 mmol, 3 eq). The mixture was stirred at 60° C. for 1 h. Upon completion, to the reaction solution was added sat. aq. NH₄Cl until pH of the solution is about 7. Then the solution was extracted with EtOAc 30 mL (10 mL*3). The organic layers were combined, washed with brine, dried over Na₂SO₄ and concentrated under reduced pressure to give methyl 2-(azetidin-1-yl)-2-methyl-propanoate (700 mg, 4.45 mmol, 40.30% yield) as a colorless oil. MS (ESI) m/z 158.4 [M+H]⁺.

Step 3: methyl 2-(azetidin-1-yl)-2-methyl propanoate

To a solution of methyl 2-(azetidin-1-yl)-2-methyl-propanoate (600 mg, 3.82 mmol, 1 eq) in MeOH (3 mL) and H₂O (3 mL) was added NaOH (152.65 mg, 3.82 mmol, 1 eq). The mixture was stirred at 15° C. for 1 h. Upon completion, the reaction mixture was dried under reduced pressure to give 2-(azetidin-1-yl)-2-methyl-propanoic acid (250 mg, crude) as a white solid. MS (ESI) m/z 144.4 [M+H]⁺.

Step 4: (1R,2S,5S)-3-[(2S)-2-[[2-(azetidin-1-yl)-2-methyl-propanoyl]amino]-3,3-dimethyl-butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide

To a solution of 2-(azetidin-1-yl)-2-methyl-propanoic acid (70 mg, 488.88 umol, 1 eq) and (1R,2S,5S)-3-[(2S)-2-amino-3,3-dimethyl-butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (210.00 mg, 488.88 umol, 1 eq) in DCM (10 mL) was added T3P (622.22 mg, 977.77 umol, 581.51 uL, 50% purity, 2 eq) and TEA (148.41 mg, 1.47 mmol, 204.14 uL, 3 eq). The mixture was stirred at 15° C. for 1 h. Upon completion, to the reaction solution was added H₂O (10 mL) and DCM (10 mL). Then the aqueous layer was separated and extracted with DCM 30 mL (10 mL*3). The organic layers were combined, dried over Na₂SO₄, and concentrated under reduced pressure to give a residue. The residue was purified by prep-HPLC (column: C18 (250*50 mm*10 um); mobile phase: [water (NH₄HCO₃)-ACN]; B %: 20%-40%, 10 min) to give (1R,2S,5S)-3-[(2S)-2-[[2-(azetidin-1-yl)-2-methyl-propanoyl]amino]-3,3-dimethyl-butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (35 mg, 63.09 umol, 12.91% yield, 100% purity) as a white solid. MS (ESI) m/z 555.4 [M+H]⁺.

¹H NMR (400 MHz, DMSO-d₆) δ=9.02 (d, J=8.4 Hz, 1H), 7.76 (s, 1H), 7.65 (d, J=9.9 Hz, 1H), 4.93 (br dd, J=5.0, 8.7 Hz, 1H), 4.33 (d, J=9.9 Hz, 1H), 4.15 (s, 1H), 3.87 (dd, J=5.4, 10.3 Hz, 1H), 3.72 (d, J=10.4 Hz, 1H), 3.11 (t, J=7.0 Hz, 4H), 2.70-2.60 (m, 1H), 2.26-2.14 (m, 1H), 2.04-1.89 (m, 4H), 1.87-1.75 (m, 1H), 1.59-1.48 (m, 1H), 1.32-1.22 (m, 1H), 1.03 (d, J=15.2 Hz, 6H), 0.94-0.87 (m, 11H), 0.86-0.69 (m, 5H), 0.62-0.50 (m, 3H).

Example 38: Synthesis of (1R,2S,5S)—N-[(1S)-1-cyan-2-[(6R)-5-oxo-4-azaspiro[2.4]heptan-6-yl]ethyl]-3-[(2S)-2-[[2-(3,3-dimethylazetidin-1-yl)acetyl]amino]-3,3-dimethyl-butanoyl]-6,6-dimethyl-3-azabicyclo[3.1.0]hexane-2-carboxamide (Compound 288)

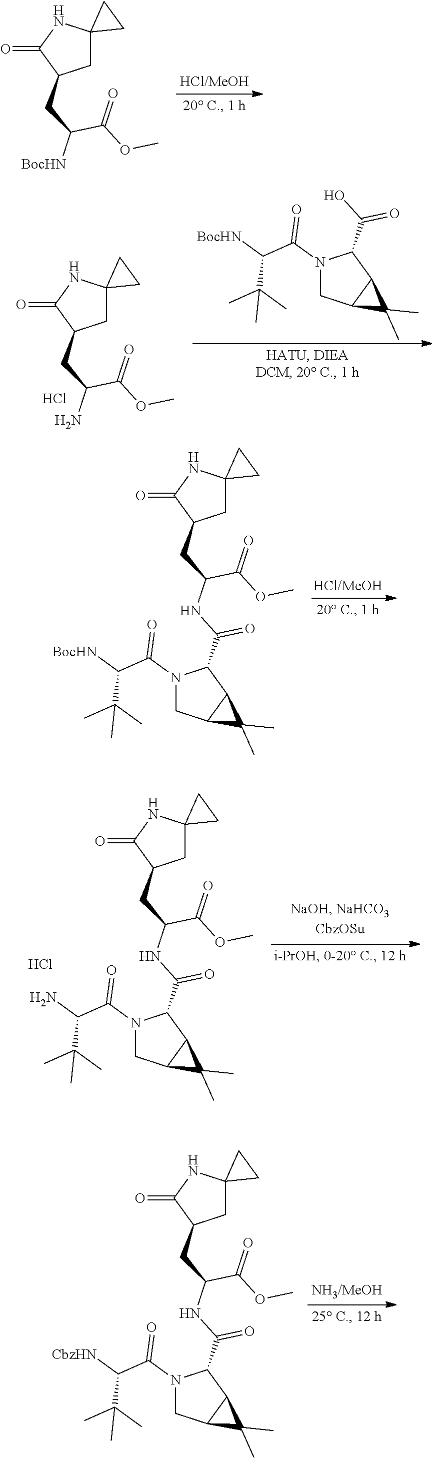

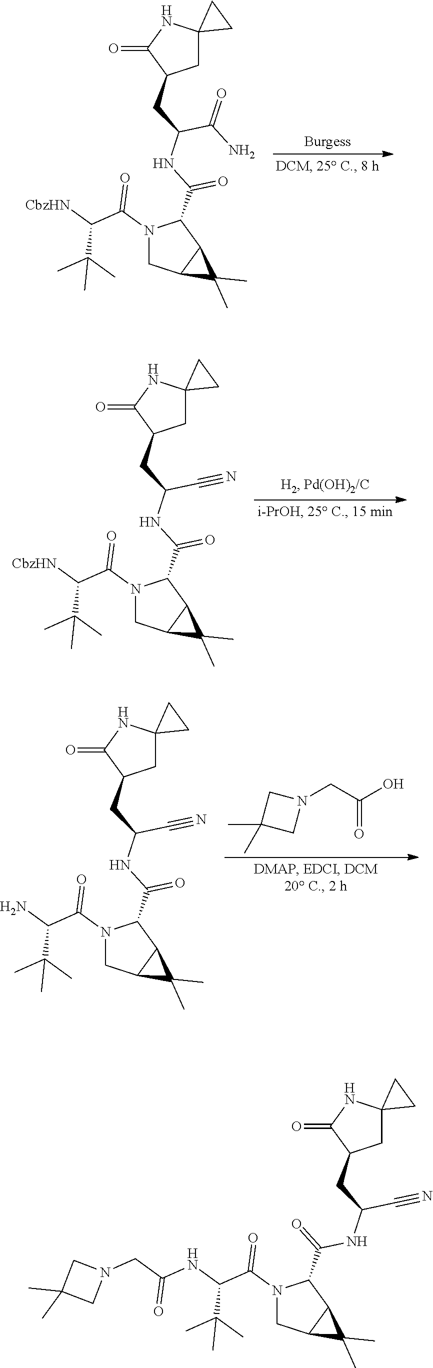

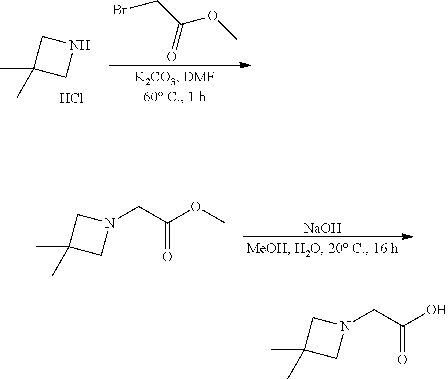

Step 1: methyl 2-(3,3-dimethylazetidin-1-yl)acetate

To a solution of methyl 2-bromoacetate (209.65 mg, 1.37 mmol, 129.42 uL, 1 eq) and 3,3-dimethylazetidine (200 mg, 1.64 mmol, 1.2 eq, HCl) in DMF (3 mL) was added K₂CO₃ (568.24 mg, 4.11 mmol, 3 eq). The mixture was stirred at 60° C. for 1 h. Upon completion, the reaction solution was added aq. NH₄C1 to pH=7. Then the solution was extracted with EA (10 mL*3). The organic layers were combined, washed with brine (10 mL*2), dried over Na₂SO₄ and concentrated under reduced pressure to give methyl 2-(3,3-dimethylazetidin-1-yl)acetate (300 mg, crude) as a colourless oil. MS (ESI) m/z 158.2 [M+H]⁺.

Step 2: 2-(3,3-dimethylazetidin-1-yl)acetic acid

To a solution of methyl 2-(3,3-dimethylazetidin-1-yl)acetate (300 mg, 1.91 mmol, 1 eq) in MeOH (2 mL) and H₂O (2 mL) was added NaOH (76.33 mg, 1.91 mmol, 1 eq). The mixture was stirred at 20° C. for 16 h. Upon completion, the reaction mixture was concentrated under reduced pressure to give 2-(3,3-dimethylazetidin-1-yl)acetic acid (290 mg, crude) as a white solid. MS (ESI) m/z 144.3 [M+H]⁺.

Step 3: methyl (2S)-2-amino-3-[(6R)-5-oxo-4-azaspiro[2.4]heptan-6-yl]propanoate

A solution of methyl (2S)-2-(tert-butoxycarbonylamino)-3-[(6R)-5-oxo-4-azaspiro[2.4]heptan-6-yl]propanoate (5 g, 16.01 mmol, 1 eq) in HCl/MeOH (100 mL) was stirred at 20° C. for 1 h. Upon completion, the reaction mixture was concentrated under reduced pressure to give methyl (2S)-2-amino-3-[(6R)-5-oxo-4-azaspiro[2.4]heptan-6-yl]propanoate (4 g, crude, HCl) as a white solid. MS (ESI) m/z 213.2 [M+H]⁺.

Step 4: methyl (2S)-2-[[(1R,2S,5S)-3-[(2S)-2-(tert-butoxycarbonylamino)-3,3-dimethyl-butanoyl]-6,6-dimethyl-3-azabicyclo[3.1.0]hexane-2-carbonyl]amino]-3-[(6R)-5-oxo-4-azaspiro[2.4]heptan-6-yl]propanoate

To a solution of methyl (2S)-2-amino-3-[(6R)-5-oxo-4-azaspiro[2.4]heptan-6-yl]propanoate (4 g, 13.35 mmol, 83% purity, 1 eq, HCl) and (1R,2S,5S)-3-[(2S)-2-(tert-butoxycarbonylamino)-3,3-dimethyl-butanoyl]-6,6-dimethyl-3-azabicyclo[3.1.0]hexane-2-carboxylic acid (4.92 g, 13.35 mmol, 1 eq) in DCM (100 mL) was added HATU (7.61 g, 20.02 mmol, 1.5 eq) and DIEA (5.18 g, 40.05 mmol, 6.98 mL, 3 eq), and then the mixture was stirred at 20° C. for 1 h. Upon completion, the reaction mixture was quenched by addition H₂O (100 mL) at 0° C., and then extracted with DCM (50 mL*3). The combined organic layers were washed with brine (100 mL), dried over Na₂SO₄, filtered and concentrated under reduced pressure to give a residue. The residue was purified by column chromatography (SiO₂, Petroleum ether:Ethyl acetate=10:1 to 1:1) to give methyl (2S)-2-[[(1R,2S,5S)-3-[(2S)-2-(tert-butoxycarbonylamino)-3,3-dimethyl-butanoyl]-6,6-dimethyl-3-azabicyclo[3.1.0]hexane-2-carbonyl]amino]-3-[(6R)-5-oxo-4-azaspiro[2.4]heptan-6-yl]propanoate (5.2 g, 8.32 mmol, 62.30% yield, 90% purity) as a white solid. MS (ESI) m/z 563.4 [M+H]⁺.

Step 5: methyl (2S)-2-[[(1R,2S,5S)-3-[(2S)-2-amino-3,3-dimethyl-butanoyl]-6,6-dimethyl-3-azabicyclo[3.1.0]hexane-2-carbonyl]amino]-3-[(6R)-5-oxo-4-azaspiro[2.4]heptan-6-yl]propanoate

A solution of methyl (2S)-2-[[(1R,2S,5S)-3-[(2S)-2-(tert-butoxycarbonylamino)-3,3-dimethyl-butanoyl]-6,6-dimethyl-3-azabicyclo[3.1.0]hexane-2-carbonyl]amino]-3-[(6R)-5-oxo-4-azaspiro[2.4]heptan-6-yl]propanoate (5.2 g, 9.24 mmol, 1 eq) in HCl/MeOH (100 mL) was stirred at 20° C. for 1 h. Upon completion, the reaction mixture was concentrated under reduced pressure to give methyl (2S)-2-[[(1R,2S,5S)-3-[(2S)-2-amino-3,3-dimethyl-butanoyl]-6,6-dimethyl-3-azabicyclo[3.1.0]hexane-2-carbonyl]amino]-3-[(6R)-5-oxo-4-azaspiro[2.4]heptan-6-yl]propanoate (4.5 g, crude, HCl) as a white solid.

Step 6: methyl (2S)-2-[[(1R,2S,5S)-3-[(2S)-2-(benzyloxycarbonylamino)-3,3-dimethyl-butanoyl]-6,6-dimethyl-3-azabicyclo[3.1.0]hexane-2-carbonyl]amino]-3-[(6R)-5-oxo-4-azaspiro[2.4]heptan-6-yl]propanoate

To a solution of methyl (2S)-2-[[(1R,2S,5S)-3-[(2S)-2-amino-3,3-dimethyl-butanoyl]-6,6-dimethyl-3-azabicyclo[3.1.0]hexane-2-carbonyl]amino]-3-[(6R)-5-oxo-4-azaspiro[2.4]heptan-6-yl]propanoate (4.5 g, 9.02 mmol, 1 eq, HCl) in i-PrOH (100 mL) was added a solution of pH=11 of NaOH (721.33 mg, 18.03 mmol, 2 eq) and NaHCO₃(2.27 g, 27.05 mmol, 1.05 mL, 3 eq) at 0° C. Then, CbzOSu (2.47 g, 9.92 mmol, 1.1 eq) was added at 0° C., and then the mixture was stirred at 20° C. for 12 h. Upon completion, the reaction mixture was quenched by addition H₂O (100 mL) at 0° C., and then extracted with EA (200 mL). The combined organic layers were washed with brine (200 mL), dried over Na₂SO₄, filtered and concentrated under reduced pressure to give a residue. The residue was purified by column chromatography (SiO₂, Petroleum ether:Ethyl acetate=10:1 to 1:3) to give methyl (2S)-2-[[(1R,2S,5S)-3-[(2S)-2-(benzyloxycarbonylamino)-3,3-dimethyl-butanoyl]-6,6-dimethyl-3-azabicyclo[3.1.0]hexane-2-carbonyl]amino]-3-[(6R)-5-oxo-4-azaspiro[2.4]heptan-6-yl]propanoate (4.5 g, 7.09 mmol, 78.61% yield, 94% purity) as a white solid. MS (ESI) m/z 597.4 [M+H]⁺.

Step 7: benzyl N-[(1S)-1-[(1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl propyl]carbamate

A solution of methyl (2S)-2-[[(1R,2S,5S)-3-[(2S)-2-(benzyloxycarbonylamino)-3,3-dimethyl-butanoyl]-6,6-dimethyl-3-azabicyclo[3.1.0]hexane-2-carbonyl]amino]-3-[(6R)-5-oxo-4-azaspiro[2.4]heptan-6-yl]propanoate (4.5 g, 7.54 mmol, 1 eq) in NH₃/MeOH (100 mL, 7 M) was stirred at 25° C. for 12 h. Upon completion, the reaction mixture was concentrated under reduced pressure to give benzyl N-[(1S)-1-[(1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl-propyl]carbamate (4.2 g, crude) as a white solid. MS (ESI) m/z 582.4 [M+H]⁺.

Step 8: benzyl N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl-propyl]carbamate

A solution of benzyl N-[(1S)-1-[(1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl-propyl]carbamate (4.2 g, 7.22 mmol, 1 eq) in DCM (100 mL) was added Burgess reagent (8.60 g, 36.10 mmol, 5 eq). The resulting mixture was stirred at 25° C. for 8 h. Upon completion, the reaction mixture was quenched by addition H₂O (100 mL) at 0° C., and then extracted with DCM (100 mL*2). The combined organic layers were washed with brine (100 mL*2), dried over Na₂SO₄, filtered and concentrated under reduced pressure to give a residue. The residue was purified by column chromatography (SiO₂, Petroleum ether:Ethyl acetate=10:1 to 1:1) to give benzyl N-[(1S)-1 [(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl-propyl]carbamate (3.7 g, 6.30 mmol, 87.27% yield, 96% purity) as a white solid. MS (ESI) m/z 564.4 [M+H]⁺.

Step 9: (1R,2S,5S)-3-[(2S)-2-amino-3,3-dimethyl-butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide

A solution of benzyl N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl-propyl]carbamate (200 mg, 354.81 umol, 1 eq) in IPA (4 mL) was added with Pd(OH)₂/C (200 mg, 20% purity) under N₂ atmosphere. The suspension was degassed and purged with H₂ for 3 times. The mixture was stirred under H₂ (15 Psi) at 25° C. for 15 min. Upon completion, the reaction mixture was concentrated under reduced pressure to give (1R,2S,5S)-3-[(2S)-2-amino-3,3-dimethyl-butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (200 mg, crude) as a colourless oil. MS (ESI) m/z 430.5 [M+H]⁺.

Step 10: (1R,2S,5S)—N-[(1S)-1-cyano-2-[(6R)-5-oxo-4-azaspiro[2.4]heptan-6-yl]ethyl]-3-[(2S)-2-[[2-(3,3-dimethylazetidin-1-yl)acetyl]amino]-3,3-dimethyl-butanoyl]-6,6-dimethyl-3-azabicyclo[3.1.0]hexane-2-carboxamide

To a solution of 2-(3,3-dimethylazetidin-1-yl)acetic acid (33.33 mg, 232.80 umol, 1 eq) and (1R,2S,5S)-3-[(2S)-2-amino-3,3-dimethyl-butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (100 mg, 232.80 umol, 1 eq) in DCM (2 mL) was added DMAP (85.32 mg, 698.40 umol, 3 eq) and EDCI (89.26 mg, 465.60 umol, 2 eq). The mixture was stirred at 20° C. for 2 h. Upon completion, the reaction mixture was quenched by addition H₂O (6 mL) at 0° C., and then extracted with DCM (10 mL*2). The combined organic layers were washed with brine (10 mL*2), dried over Na₂SO₄, filtered and concentrated under reduced pressure to give a residue. The residue was purified by prep-HPLC (column: Waters Xbridge Prep OBD C18 150*40 mm*10 um; mobile phase: [water(NH₄HCO₃)-ACN]; B %: 20% 50%, 8 min) to give (1R,2S,5S)—N-[(1S)-1-cyano-2-[(6R)-5-oxo-4-azaspiro[2.4]heptan-6-yl]ethyl]-3-[(2S)-2-[[2-(3,3-dimethylazetidin-1-yl)acetyl]amino]-3,3-dimethyl-butanoyl]-6,6-dimethyl-3-azabicyclo[3.1.0]hexane-2-carboxamide (15 mg, 26.50 umol, 11.38% yield, 98% purity) was obtained as a white solid. MS (ESI) m/z 555.5 [M+H]⁺.

¹H NMR (400 MHz, DMSO-d₆) δ=8.99 (d, J=8.4 Hz, 1H), 7.73 (s, 1H), 7.47 (d, J=9.4 Hz, 1H), 4.93-4.63 (m, 1H), 4.35-4.26 (m, 1H), 4.12 (s, 1H), 3.87-3.83 (m, 1H), 3.72 (d, J=10.5 Hz, 1H), 3.00 (s, 2H), 2.96-2.88 (m, 4H), 2.67-2.60 (m, 1H), 2.21 2.08 (m, 1H), 2.01-1.89 (m, 2H), 1.83-1.76 (m, 1H), 1.54-1.44 (m, 1H), 1.27 (d, J=7.6 Hz, 1H), 1.14 (s, 6H), 1.00 (s, 3H), 0.91 (s, 9H), 0.81 (s, 3H), 0.74-0.68 (m, 1H), 0.56-0.46 (m, 3H).

Example 39: Synthesis of (1R,2S,5S)—N-[(1S)-1-cyan-2-[(6R)-4-methyl-5-oxo-4-azaspiro[2.4]heptan-6-yl]ethyl]-3-[(2S)-3,3-dimethyl-2-[(2,2,2-trifluoroacetyl)amino]butanoyl]-6,6-dimethyl-3-azabicyclo[3.1.0]hexane-2-carboxamide (Compound 289)

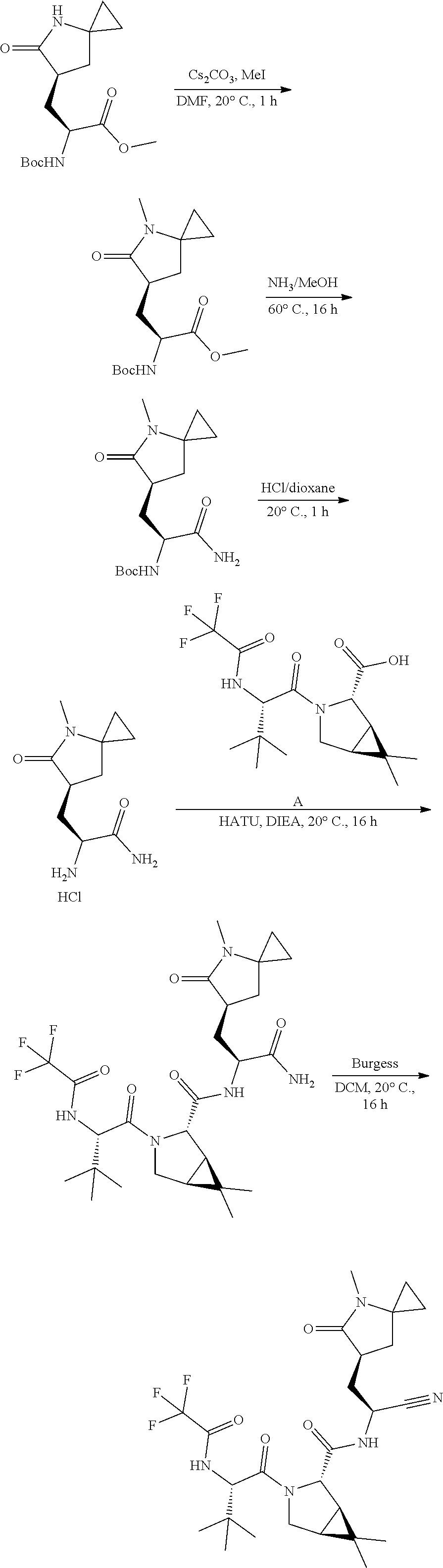

Step 1: methyl(2S)-2-(tert-butoxycarbonylamino)-3-[(6R)-4-methyl-5-oxo-4-azaspiro[2.4]heptan-6-yl]propanoate

A solution of methyl (2S)-2-(tert-butoxycarbonylamino)-3-[(6R)-5-oxo-4-azaspiro[2.4]heptan-6-yl]propanoate (1 g, 3.20 mmol, 1 eq) in DMF (10 mL) was added Cs₂CO₃ (3.13 g, 9.60 mmol, 3 eq) and Mel (908.81 mg, 6.40 mmol, 398.60 uL, 2 eq). The mixture was stirred at 20° C. for 1 h. Upon completion, the reaction mixture was diluted by addition H₂O (10 mL), and then extracted with DCM (10 mL*3). The combined organic layers were washed with brine (10 mL), dried over Na₂SO₄, filtered and concentrated under reduced pressure to give a residue. The residue was purified by prep-HPLC (column: Welch Xtimate C18 250*70 mm #10 um; mobile phase: [water (NH₄HCO₃)-ACN]; B %: 25% 55%, 20 min) to give the product methyl (2S)-2-(tert-butoxycarbonylamino)-3-[(6R)-4-methyl-5-oxo-4-azaspiro[2.4]heptan-6-yl]propanoate (600 mg, 1.84 mmol, 57.42% yield) as a yellow oil. MS (ESI) m/z 327.3 [M+H]⁺

Step 2: tert-butylN-[(1S)-2-amino-1-[[(6R)-4-methyl-5-oxo-4-azaspiro[2.4]heptan-6-yl]methyl]-2-oxo-ethyl]carbamate

A solution of methyl (2S)-2-(tert-butoxycarbonylamino)-3-[(6R)-4-methyl-5-oxo-4-azaspiro[2.4]heptan-6-yl]propanoate (550 mg, 1.69 mmol, 1 eq) in NH₃/MeOH (7 M, 10 mL, 41.54 eq) was stirred at 60° C. for 16 h. Upon completion, the mixture was concentrated under the reduced pressure to give tert-butyl N-[(1S)-2-amino-1-[[(6R)-4-methyl-5-oxo-4-azaspiro[2.4]heptan-6-yl]methyl]-2-oxo-ethyl]carbamate (500 mg, crude) as a yellow solid. MS (ESI) m/z 312.2 [M+H]⁺

Step 3: (2S)-2-amino-3-[(6R)-4-methyl-5-oxo-4-azaspiro[2.4]heptan-6-yl]propanamide

A solution of tert-butyl N-[(1S)-2-amino-1-[[(6R)-4-methyl-5-oxo-4-azaspiro[2.4]heptan-6-yl]methyl]-2-oxo-ethyl]carbamate (500 mg, 1.61 mmol, 1 eq) in HCl/dioxane (4 M, 10.00 mL, 24.91 eq) was stirred at 20° C. for 1 h. Upon completion, the mixture was concentrated under the reduced pressure to give (2S)-2-amino-3-[(6R)-4-methyl-5-oxo-4-azaspiro[2.4]heptan-6-yl]propanamide (400 mg, crude, HCl) as a yellow solid. MS (ESI) m/z 212.1 [M+H]⁺

Step 4: (1R,2S,5S)—N-[(1S)-2-amino-1-[[(6R)-4-methyl-5-oxo-4-azaspiro[2.4]heptan-6-yl]methyl]-2-oxo-ethyl]-3-[(2S)-3,3-dimethyl-2-[(2,2,2-trifluoroacetyl)amino]butanoyl]-6,6-dimethyl-3-azabicyclo[3.1.0]hexane-2-carboxamide

To a solution of (1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carboxylic acid (294.17 mg, 807.36 umol, 1 eq) in DCM (5 mL) was added DIEA (208.69 mg, 1.61 mmol, 281.25 uL, 2 eq), and then HATU (368.38 mg, 968.83 umol, 1.2 eq) was added. The mixture was stirred at 20° C. for 1 h. (2S)-2-amino-3-[(6R)-4-methyl-5-oxo-4-azaspiro[2.4]heptan-6-yl]propanamide (200 mg, 807.36 umol, 1 eq, HCl) was added to the mixture and stirred at 20° C. for 15 h. Upon completion, the reaction mixture was diluted by addition H₂O (20 mL), and then extracted with DCM (10 mL*3). The combined organic layers were washed with brine (10 mL), dried over Na₂SO₄, filtered and concentrated under reduced pressure to give a residue. The residue was purified by column chromatography (SiO₂, Petroleum ether:Ethyl acetate=5:1 to 0:1) to give (1R,2S,5S)—N-[(1S)-2-amino-1-[[(6R)-4-methyl-5-oxo-4-azaspiro[2.4]heptan-6-yl]methyl]-2-oxo-ethyl]-3-[(2S)-3,3-dimethyl-2-[(2,2,2-trifluoroacetyl)amino]butanoyl]-6,6-dimethyl-3-azabicyclo[3.1.0]hexane-2-carboxamide (100 mg, 179.34 umol, 22.21% yield) as a yellow solid. MS (ESI) m/z 558.4 [M+H]⁺

Step 5: (1R,2S,5S)—N-[(1S)-1-cyano-2-[(6R)-4-methyl-5-oxo-4-azaspiro[2.4]heptan-6-yl]ethyl]-3-[(2S)-3,3-dimethyl-2-[(2,2,2-trifluoroacetyl)amino]butanoyl]-6,6-dimethyl-3-azabicyclo[3.1.0]hexane-2-carboxamide

A solution of (1R,2S,5S)—N-[(1S)-2-amino-1-[[(6R)-4-methyl-5-oxo-4-azaspiro[2.4]heptan-6-yl]methyl]-2-oxo-ethyl]-3-[(2S)-3,3-dimethyl-2-[(2,2,2-trifluoroacetyl)amino]butanoyl]-6,6-dimethyl-3-azabicyclo[3.1.0]hexane-2-carboxamide (100 mg, 179.34 umol, 1 eq) in DCM (5 mL) was added with Burgess reagent (128.21 mg, 538.02 umol, 3 eq). The mixture was stirred at 20° C. for 16 h. Upon completion, the reaction mixture was diluted by addition H₂O (20 mL), and then extracted with DCM (10 mL*3). The combined organic layers were washed with brine (10 mL), dried over Na₂SO₄, filtered and concentrated under reduced pressure to give a residue. The residue was purified by prep-HPLC (column: Waters Xbridge Prep OBD C18 150*40 mm*10 um; mobile phase: [water (NH₄HCO₃)-ACN]; B %: 35%-65%, 8 min) to give (1R,2S,5S)—N-[(1S)-1-cyano-2-[(6R)-4-methyl-5-oxo-4-azaspiro[2.4]heptan-6-yl]ethyl]-3-[(2S)-3,3-dimethyl-2-[(2,2,2-trifluoroacetyl)amino]butanoyl]-6,6-dimethyl-3-azabicyclo[3.1.0]hexane-2-carboxamide (15 mg, 27.80 umol, 15.50% yield, 100% purity) as a white solid. MS (ESI) m/z 540.2 [M+H]⁺

¹H NMR (400 MHz, DMSO-d₆) δ=9.40 (br d, J=7.0 Hz, 1H), 9.04 (d, J=8.7 Hz, 1H), 4.96 (ddd, J=5.1, 8.6, 10.8 Hz, 1H), 4.40 (br d, J=7.0 Hz, 1H), 4.24-4.12 (m, 1H), 3.90 (dd, J=5.5, 10.4 Hz, 1H), 3.70 (d, J=10.4 Hz, 1H), 2.87-2.68 (m, 1H), 2.52 (br s, 1H), 2.44-2.37 (m, 3H), 2.36-2.30 (m, 1H), 2.23 (ddd, J=4.3, 10.9, 13.4 Hz, 1H), 1.96 (d, J=9.1 Hz, 1H), 1.80 (ddd, J=5.3, 11.0, 13.4 Hz, 1H), 1.57 (dd, J=5.5, 7.4 Hz, 1H), 1.36-0.80 (m, 17H), 0.68-0.42 (m, 2H)

¹H NMR (400 MHz, MeOD-d_{4 8=5.04} (dd, J=5.1, 10.8 Hz, 1H), 4.58-4.48 (m, 1H), 4.31-4.21 (m, 1H), 4.02 (br dd, J=5.5, 10.3 Hz, 1H), 3.86 (br d, J=10.4 Hz, 1H), 3.00-2.31 (m, 5H), 2.25-2.12 (m, 1H), 2.11-2.08 (m, 1H), 2.07-1.98 (m, 1H), 1.93 (ddd, J=5.1, 10.3, 13.8 Hz, 1H), 1.68-1.57 (m, 1H), 1.29-0.87 (m, 17H), 0.78 (dt, J=2.2, 7.3 Hz, 2H)

Example 40: Synthesis of (1R,2S,5S)-N—((S)-1-cyan-2-((R)-5,5-dimethyl-2-oxopyrrolidin-3-yl)ethyl)-3-((2S,3R)-3-methoxy-2-(2,2,2-trifluoroacetamido)butanoyl)-6,6-dimethyl-3-azabicyclo[3.1.0]hexane-2-carboxamide (Compound 290)

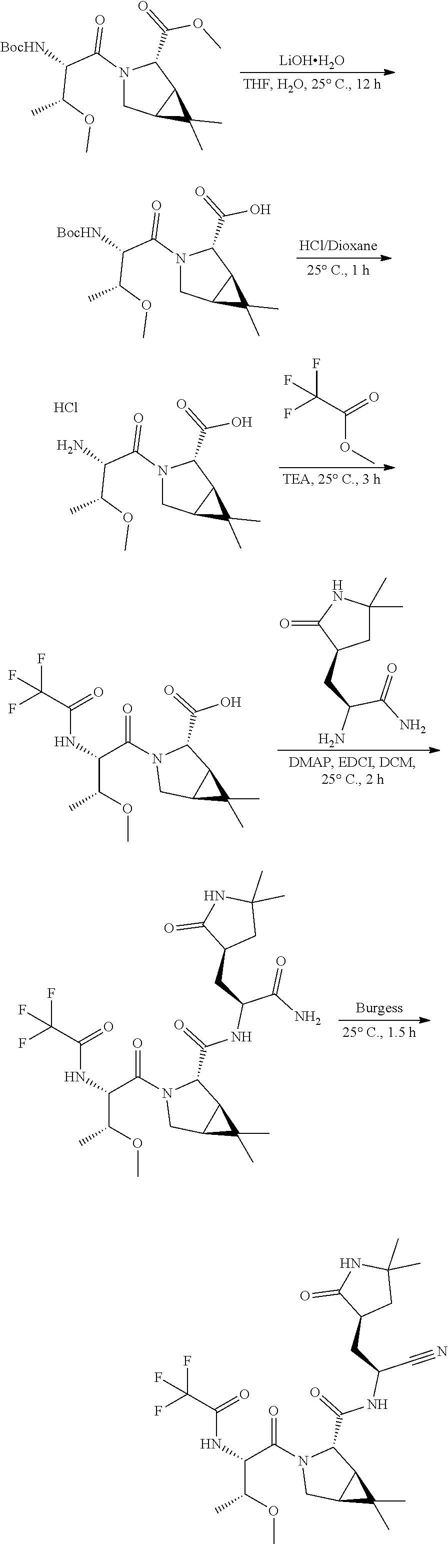

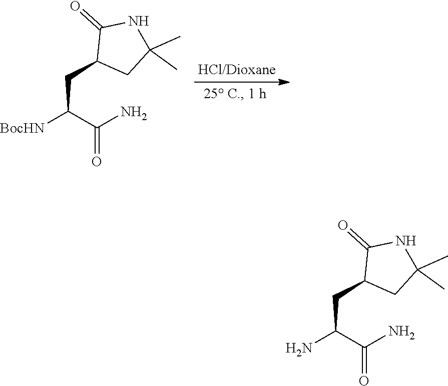 [SMILES: CC1(C)C[C@@H](C[C@H](NC(=O)OC(C)(C)C)C(N)=O)C(=O)N1 ; CC1(C)C[C@@H](C[C@H](N)C(N)=O)C(=O)N1]

Step 1: (S)-2-amino-3-((R)-5,5-dimethyl-2-oxopyrrolidin-3-yl)propanamide

A solution of tert-butyl ((S)-1-amino-3-((R)-5,5-dimethyl-2-oxopyrrolidin-3-yl)-1-oxopropan-2-yl)carbamate (500 mg, 1.67 mmol, 1 eq) in HCl/dioxane (4 M, 5.00 mL, 11.97 eq) was stirred at 25° C. for 1 h. Upon completion, the resulting solution was concentrated in vacuum (40° C.) to give (S)-2-amino-3-((R)-5,5-dimethyl-2-oxopyrrolidin-3-yl)propanamide (395 mg, crude, HCl) as a white solid.

Step 2: (1R,2S,5S)-3-((2S,3R)-2-((tert-butoxycarbonyl)amino)-3-methoxybutanoyl)-6,6-dimethyl-3-azabicyclo[3.1.0]hexane-2-carboxylic acid

To a solution of (1R,2S,5S)-methyl 3-((2S,3R)-2-((tert-butoxycarbonyl)amino)-3-methoxybutanoyl)-6,6-dimethyl-3-azabicyclo[3.1.0]hexane-2-carboxylate (600 mg, 1.56 mmol, 1 eq) in THE (3 mL) and H₂O (1 mL) was added LiOH.H₂O (196.45 mg, 4.68 mmol, 3 eq), and then the mixture was stirred at 25° C. for 12 h. Upon completion, the mixture was adjust pH to 3-4 by HCl (1 N), and extracted with EA (20 mL*2). The combined organic layers were washed with brine (20 mL*2), dried over Na₂SO₄, filtered and concentrated under reduced pressure to give (1R,2S,5S)-3-((2S,3R)-2-((tert-butoxycarbonyl)amino)-3-methoxybutanoyl)-6,6-dimethyl-3-azabicyclo[3.1.0]hexane-2-carboxylic acid (560 mg, crude) as a white solid.

Step 3: (1R,2S,5S)-3-((2S,3R)-2-amino-3-methoxybutanoyl)-6,6-dimethyl-3-azabicyclo[3.1.0]hexane-2-carboxylic acid

A solution of (1R,2S,5S)-3-((2S,3R)-2-((tert-butoxycarbonyl)amino)-3-methoxybutanoyl)-6,6-dimethyl-3-azabicyclo[3.1.0]hexane-2-carboxylic acid (930 mg, 2.51 mmol, 1 eq) in HCl/dioxane (4 M, 9.30 mL, 14.82 eq) was stirred at 25° C. for 1 h. Upon completion, the resulting solution was concentrated in vacuum (40° C.) to give (1R,2S,5S)-3-((2S,3R)-2-amino-3-methoxybutanoyl)-6,6-dimethyl-3-azabicyclo[3.1.0]hexane-2-carboxylic acid (760 mg, crude, HCl) as a white solid.

Step 4: (1R,2S,5S)-3-((2S,3R)-3-methoxy-2-(2,2,2-trifluoroacetamido)butanoyl)-6,6-dimethyl-3-azabicyclo[3.1.0]hexane-2-carboxylic acid

To a solution of (1R,2S,5S)-3-((2S,3R)-2-amino-3-methoxybutanoyl)-6,6-dimethyl-3-azabicyclo[3.1.0]hexane-2-carboxylic acid (760 mg, 2.48 mmol, 1 eq, HCl) in methyl 2,2,2-trifluoroacetate (3.17 g, 24.77 mmol, 2.50 mL, 10 eq) was added TEA (752.03 mg, 7.43 mmol, 1.03 mL, 3 eq), and then the mixture was stirred for 3 h at 25° C. Upon completion, the resulting solution was concentrated in vacuum, then adjust pH to 2-3 by HCl (5 mL), and extracted with EA (20 mL*2). The combined organic layers were washed with brine (20 mL*2), dried over Na₂SO₄. The crude product was triturated with PE:EA=20:1 at 25° C. for 1 h to give (1R,2S,5S)-3-((2S,3R)-3-methoxy-2-(2,2,2-trifluoroacetamido)butanoyl)-6,6-dimethyl-3-azabicyclo[3.1.0]hexane-2-carboxylic acid (750 mg, 2.05 mmol, 82.64% yield) as a white solid.

Step 5: (1R,2S,5S)-N—((S)-1-amino-3-((R)-5,5-dimethyl-2-oxopyrrolidin-3-yl)-1-oxopropan-2-yl)-3-((2S,3R)-3-methoxy-2-(2,2,2-trifluoroacetamido)butanoyl)-6,6-dimethyl-3-azabicyclo[3.1.0]hexane-2-carboxamide

To a solution of (1R,2S,5S)-3-((2S,3R)-3-methoxy-2-(2,2,2-trifluoroacetamido)butanoyl)-6,6-dimethyl-3-azabicyclo[3.1.0]hexane-2-carboxylic acid (750 mg, 2.05 mmol, 1 eq) in DCM (7 mL) was added (S)-2-amino-3-((R)-5,5-dimethyl-2-oxopyrrolidin-3-yl)propanamide (643.43 mg, 2.05 mmol, 75% purity, 1 eq, HCl), EDCI (784.95 mg, 4.09 mmol, 2 eq) and DMAP (750.35 mg, 6.14 mmol, 3 eq). The resulting mixture was stirred at 25° C. for 2 h. Upon completion, the resulting solution was poured into brine (20 mL), extracted with DCM (30 mL*2), the combined organic phase was washed brine (30 mL*3), dried over Na₂SO₄, filtered and concentrated to give a crude product. The residue was purified by column chromatography (SiO₂, Petroleum ether:Ethyl acetate=1:1 to EA:MeOH=10:1) to give (1R,2S,5S)-N—((S)-1-amino-3-((R)-5,5-dimethyl-2-oxopyrrolidin-3-yl)-1-oxopropan-2-yl)-3-((2S,3R)-3-methoxy-2-(2,2,2-trifluoroacetamido)butanoyl)-6,6-dimethyl-3-azabicyclo[3.1.0]hexane-2-carboxamide (240 mg, 438.30 umol, 21.41% yield) as a light yellow oil.

Step 6: (1R,2S,5S)-N—((S)-1-cyan-2-((R)-5,5-dimethyl-2-oxopyrrolidin-3-yl)ethyl)-3-((2S,3R)-3-methoxy-2-(2,2,2-trifluoroacetamido)butanoyl)-6,6-dimethyl-3-azabicyclo[3.1.0]hexane-2-carboxamide

To a solution of (1R,2S,5S)-N—((S)-1-amino-3-((R)-5,5-dimethyl-2-oxopyrrolidin-3-yl)-1-oxopropan-2-yl)-3-((2S,3R)-3-methoxy-2-(2,2,2-trifluoroacetamido)butanoyl)-6,6-dimethyl-3-azabicyclo[3.1.0]hexane-2-carboxamide (240 mg, 438.30 umol, 1 eq) in DCM (3 mL) was added Burgess reagent (208.90 mg, 876.61 umol, 2 eq), and then the mixture was stirred at 25° C. for 1.5 h. Upon completion, the resulting solution was quenched with H₂O (0.2 mL), then was concentrated in vacuum (25° C.). The residue was purified by prep-HPLC (column: Phenomenex C18 75*30 mm*3 um; mobile phase: [water(NH₄HCO₃)-ACN]; B %: 30%-50%, 8 min) to give (1R,2S,5S)-N—((S)-1-cyano-2-((R)-5,5-dimethyl-2-oxopyrrolidin-3-yl)ethyl)-3-((2S,3R)-3-methoxy-2-(2,2,2-trifluoroacetamido)butanoyl)-6,6-dimethyl-3-azabicyclo[3.1.0]hexane-2-carboxamide (132.20 mg, 249.65 umol, 56.96% yield, 100% purity) as a white solid. MS (ESI) m/z 530.2[M+H]⁺

¹H NMR (400 MHz, DMSO-d₆) δ=10.03-9.26 (m, 1H), 9.00 (br d, J=8.6 Hz, 1H), 7.87-7.74 (m, 1H), 4.99-4.80 (m, 1H), 4.33 (br d, J=9.0 Hz, 1H), 4.12 (s, 1H), 3.95 3.88 (m, 1H), 3.88-3.79 (m, 1H), 3.64-3.56 (m, 1H), 3.25-3.19 (m, 3H), 2.59-2.53 (m, 1H), 2.20-2.09 (m, 1H), 2.01-1.92 (m, 1H), 1.77-1.67 (m, 1H), 1.60-1.47 (m, 2H), 1.37 1.30 (m, 1H), 1.19 (s, 3H), 1.16-1.10 (m, 6H), 1.05-1.01 (m, 3H), 0.91-0.85 (m, 3H)

Example 41: Synthesis of (1R,2S,5S)—N-[(1S)-1-cyano-2-[(6R)-5-oxo-4-azaspiro[2.4]heptan-6-yl]ethyl]-3-[(2S)-3,3-dimethyl-2-[[1-(trifluoromethyl)cyclobutanecarbonyl]amino]butanoyl]-6,6-dimethyl-3-azabicyclo[3.1.0]hexane-2-carboxamide (Compound 291)

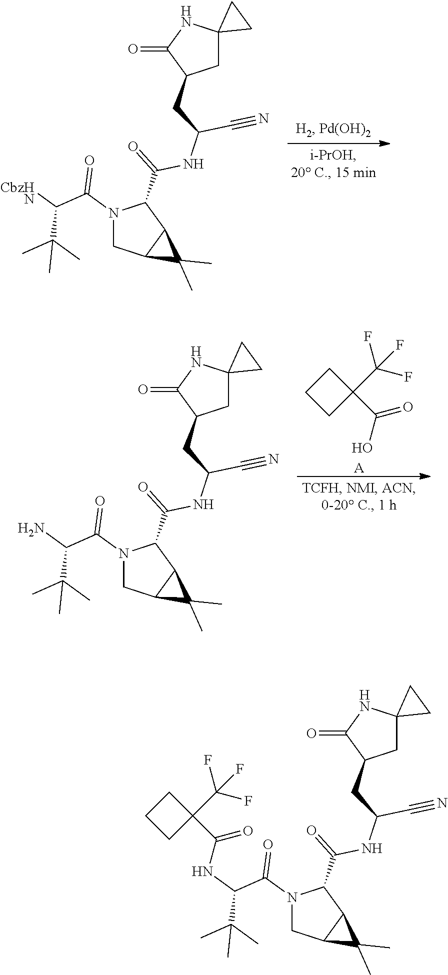

Step 1: (1R,2S,5S)-3-[(2S)-2-amino-3,3-dimethyl-butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide

To a solution of benzyl N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl-propyl]carbamate (300 mg, 510.92 umol, 96% purity, 1 eq) in isopropyl alcohol (15 mL) was added Pd(OH)₂ (300 mg, 427.24 umol, 20% purity, 8.36e-1 eq) under N₂. The suspension was degassed under vacuum and purged with H₂ several times. The mixture was stirred under H₂ (15 Psi) at 20° C. for 15 min. The reaction mixture was filtered and concentrated under reduced pressure to give (1R,2S,5S)-3-[(2S)-2-amino-3,3-dimethyl-butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (250 mg, crude) as a white solid. MS (ESI) m/z 430.4 [M+H]⁺.

Step 2: 2-fluoroethyl N-[(1S)-1-[(1R,2S,5S)-2-[((1S)-2-amino-2-oxo-1-[[(6R)-5-oxo-4-azaspiro[2.4]heptan-6-yl/methyl/ethyl]carbamoyl]-6,6-dimethyl-3-azabicyclo[3.1.0]hexane-3-carbonyl]-2,2-dimethyl propyl/carbamate

To a solution of (1R,2S,5S)-3-[(2S)-2-amino-3,3-dimethyl-butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (233 mg, 542.42 umol, 1 eq), 1-(trifluoromethyl)cyclobutanecarboxylic acid (91.19 mg, 542.42 umol, 1 eq), and 1-methylimidazole (133.60 mg, 1.63 mmol, 129.71 uL, 3 eq) in ACN (5 mL) was added [chloro(dimethylamino)methylene]-dimethyl-ammonium; hexafluorophosphate (304.38 mg, 1.08 mmol, 2 eq) at 0° C. The mixture was stirred at 20° C. for 1 h. Upon completion, the reaction mixture was poured into H₂O 30 mL at 20° C., and then extracted with DCM(30 mL*3). The combined organic layers were washed with brine 30 mL, dried over Na₂SO₄, filtered and concentrated under reduced pressure to give a residue. The crude product was purified by prep-HPLC(column: Waters Xbridge Prep OBD C18 150*40 mm*10 um; mobile phase: [water(NH₄HCO₃)-ACN]; B %: 35%-65%, 8 min) to give (1R,2S,5S)—N-[(1S)-1-cyano-2-[(6R)-5-oxo-4-azaspiro[2.4]heptan-6-yl]ethyl]-3-[(2S)-3,3-dimethyl-2-[[1-(trifluoromethyl)cyclobutanecarbonyl]amino]butanoyl]-6,6-dimethyl-3-azabicyclo[3.1.0]hexane-2-carboxamide (73.2 mg, 126.28 umol, 23.28% yield, 100% purity) as a white solid. MS (ESI) m/z 580.4 [M+H]⁺.

¹H NMR (400 MHz, DMSO-d₆) δ=8.99 (d, J=8.6 Hz, 1H), 7.76-7.67 (m, 2H), 4.93 (ddd, J=5.3, 8.6, 10.6 Hz, 1H), 4.52 (d, J=9.3 Hz, 1H), 4.15 (s, 1H), 3.89 (dd, J=5.4, 10.3 Hz, 1H), 3.72 (d, J=10.4 Hz, 1H), 2.69-2.62 (m, 1H), 2.40-2.33 (m, 3H), 2.23-2.15 (m, 1H), 2.05-1.68 (m, 6H), 1.54 (dd, J=5.5, 7.5 Hz, 1H), 1.29 (d, J=7.6 Hz, 1H), 1.02 (s, 3H), 0.94 (s, 9H), 0.82 (s, 3H), 0.75-0.71 (m, 1H), 0.58-0.52 (m, 3H).

Example 42: Synthesis of (1R,2S,5S)-N—((S)-1-cyan-2-((R)-5-oxo-4-azaspiro[2.4]heptan-6-yl)ethyl)-3-((2S)-2-(2,2-difluoro-1-(tluoromethyl)cydopropanecarboxamido)-3,3-dimethylbutanoyl)-6,6-dimethyl-3-azabicyclo[3.1.0]hexane-2-carboxamide (Compound 292)

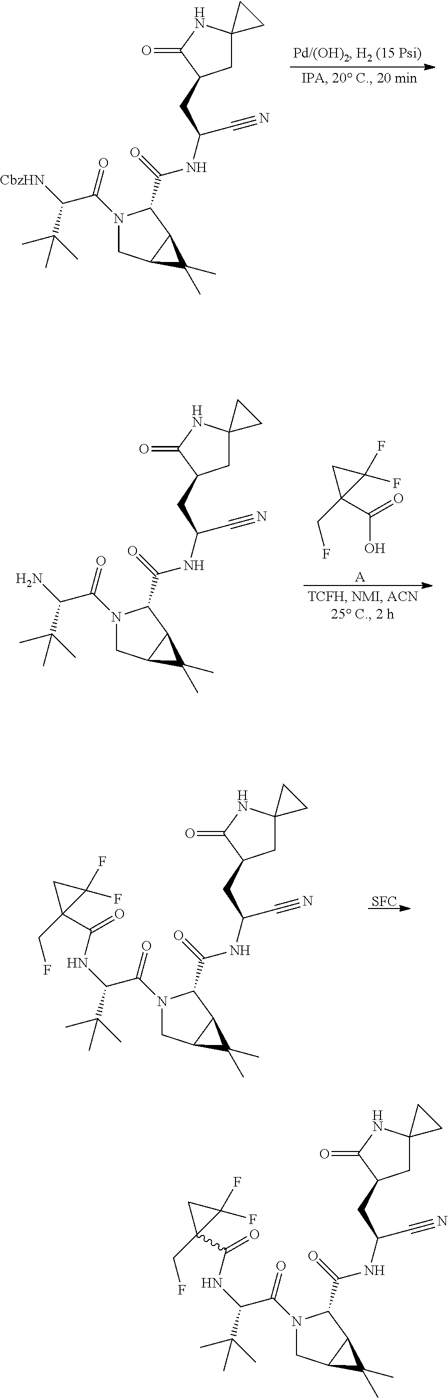

Step 1: (1R,2S,5S)-3-[(2S)-2-amino-3,3-dimethyl-butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide

To a solution of benzyl N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl-propyl]carbamate (300 mg, 532.21 umol, 1 eq) in IPA (2 mL) was added Pd(OH)₂ (300.00 mg, 2.14 mmol, 4.01 eq). The mixture was stirred at 25° C. for 20 min under H₂ (15 Psi). Upon completion, the mixture was concentrated under the reduced pressure to give the product (1R,2S,5S)-3-[(2S)-2-amino-3,3-dimethyl-butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (162 mg, crude) as a white solid.

Step 2: (1R,2S,5S)—N-[(1S)-1-cyano-2-[(6R)-5-oxo-4-azaspiro[2.4]heptan-6-yl]ethyl]-3-[(2S)-2-[[2,2-difluoro-1-(fluoromethyl)cyclopropanecarbonyl]amino]-3,3-dimethyl-butanoyl]-6,6-dimethyl-3-azabicyclo[3.1.0]hexane-2-carboxamide

To a solution of 2,2-difluoro-1-(fluoromethyl)cyclopropanecarboxylic acid (58 mg, 376.41 umol, 1 eq) in ACN (5 mL) was added (1R,2S,5S)-3-[(2S)-2-amino-3,3-dimethyl-butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (161.69 mg, 376.41 umol, 1 eq), then added [chloro(dimethylamino)methylene]-dimethyl-ammonium; hexafluorophosphate (126.74 mg, 451.69 umol, 1.2 eq) and 1-methylimidazole (108.16 mg, 1.32 mmol, 105.01 uL, 3.5 eq). The mixture was stirred at 25° C. for 2 h. Upon completion, the residue was quenched by H₂O (5 mL) and was extracted with EA (5 mL*3). The combined organic phase was washed with brine (2 mL), dried with anhydrous Na₂SO₄, filtered and concentrated under reduced pressure to give a residue. The residue was purified by prep-HPLC (column: Waters Xbridge BEH C18 100*30 mm*10 um; mobile phase: [water (NH₄HCO₃)-ACN]; B %: 35%-65%, 8 min) to give the product (1R,2S,5S)—N-[(1S)-1-cyano-2-[(6R)-5-oxo-4-azaspiro[2.4]heptan-6-yl]ethyl]-3-[(2S)-2-[[2,2-difluoro-1-(fluoromethyl)cyclopropanecarbonyl]amino]-3,3-dimethyl-butanoyl]-6,6-dimethyl-3-azabicyclo[3.1.0]hexane-2-carboxamide (65 mg, 114.92 umol, 30.53% yield) as a white solid. MS (ESI) m/z 566.2 [M+H]⁺

Step 3: (1R,2S,5S)-N—((S)-1-cyano-2-((R)-5-oxo-4-azaspiro[2.4]heptan-6-yl)ethyl)-3-((2S)-2-(2,2-difluoro-1-(fluoromethyl)cyclopropanecarboxamido)-3,3-dimethylbutanoyl)-6,6-dimethyl-3-azabicyclo[3.1.0]hexane-2-carboxamide

(1R,2S,5S)—N-[(1S)-1-cyano-2-[(6R)-5-oxo-4-azaspiro[2.4]heptan-6-yl]ethyl]-3-[(2S)-2-[[2,2-difluoro-1-(fluoromethyl)cyclopropanecarbonyl]amino]-3,3-dimethyl-butanoyl]-6,6-dimethyl-3-azabicyclo[3.1.0]hexane-2-carboxamide (65 mg, 114.92 umol) was purified by SPC to give the product (1R,2S,5S)-N—((S)-1-cyano-2-((R)-5-oxo-4-azaspiro[2.4]heptan-6-yl)ethyl)-3-((2S)-2-(2,2-difluoro-1-(fluoromethyl)cyclopropanecarboxamido)-3,3-dimethylbutanoyl)-6,6-dimethyl-3-azabicyclo[3.1.0]hexane-2-carboxamide (Isomer 1) (18.1 mg, 32.00 umol, 27.85% yield) as a white solid. MS (ESI) m/z 566.2 [M+H]⁺

¹H NMR (400 MHz, DMSO-d₆) δ=9.00 (d, J=8.4 Hz, 1H), 8.18 (br d, J=8.8 Hz, 1H), 7.75 (s, 1H), 5.15-4.79 (m, 2H), 4.55-4.31 (m, 2H), 4.14 (s, 1H), 3.88 (dd, J=5.4, 10.2 Hz, 1H), 3.65 (d, J=10.4 Hz, 1H), 2.73-2.62 (m, 1H), 2.23-2.07 (m, 2H), 2.04-1.86 (m, 3H), 1.81 (ddd, J=5.0, 10.6, 13.4 Hz, 1H), 1.54 (dd, J=5.6, 7.4 Hz, 1H), 1.32-1.25 (m, 1H), 1.07-0.87 (m, 12H), 0.85-0.57 (m, 5H), 0.56-0.51 (m, 2H)

(1R,2S,5S)—N-[(1S)-1-Cyano-2-[(6R)-5-oxo-4-azaspiro[2.4]heptan-6-yl]ethyl]-3-[(2S)-2-[[2,2-difluoro-1-(fluoromethyl)cyclopropanecarbonyl]amino]-3,3-dimethyl-butanoyl]-6,6-dimethyl-3-azabicyclo[3.1.0]hexane-2-carboxamide (Isomer 2) (9.01 mg, 15.93 umol, 13.86% yield) was obtained. MS (ESI) m/z 566.2 [M+H]⁺

¹H NMR (400 MHz, DMSO-d₆) δ=9.00 (d, J=8.4 Hz, 1H), 8.43 (br d, J=9.2 Hz, 1H), 7.74 (s, 1H), 5.27-5.06 (m, 1H), 4.93 (ddd, J=5.0, 8.6, 10.6 Hz, 1H), 4.53-4.34 (m, 2H), 4.16-4.11 (m, 1H), 3.92-3.83 (m, 1H), 3.76 (d, J=10.4 Hz, 1H), 2.75-2.60 (m, 1H), 2.26-2.10 (m, 2H), 2.04-1.75 (m, 3H), 1.70-1.68 (m, 1H), 1.52 (dd, J=5.4, 7.4 Hz, 1H), 1.28 (d, J=7.6 Hz, 1H), 1.06-0.86 (m, 12H), 0.82-0.67 (m, 4H), 0.64-0.44 (m, 3H)

Example 43: Synthesis of (1R, 2S, 5S)—N-[(1S)-1-cyan-2-[(6R)-5-oxo-4-azaspiro[2.4]heptan-6-yl]ethyl]-3-[(2S)-3,3-dimethyl-2-[(3,3,3-trifluoro-2,2-dimethyl-propanoyl)amino]butanoyl]-6,6-dimethyl-3-azabicyclo[3.1.0]hexane-2-carboxamide (Compound 293)

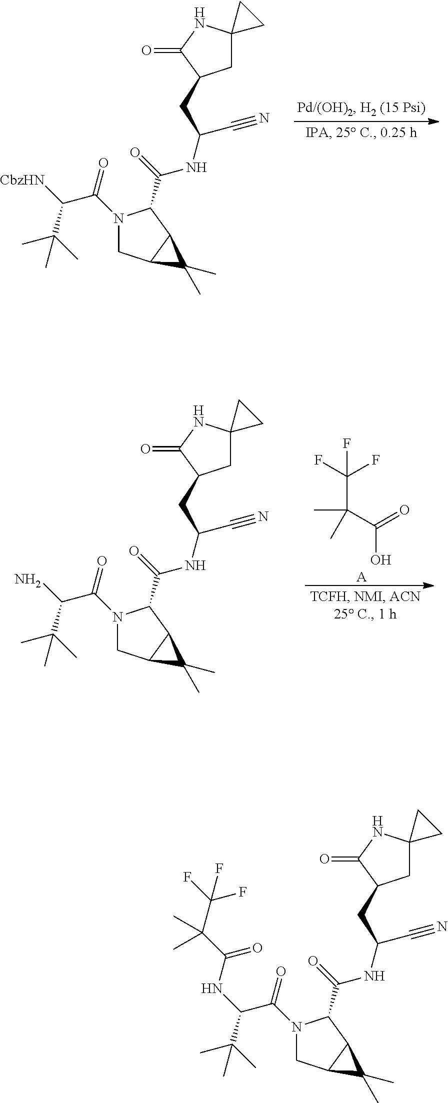

Step 1: (1R,2S,5S)-3-[(2S)-2-amino-3,3-dimethyl-butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide

A solution of benzyl N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl-propyl]carbamate (300 mg, 532.21 umol, 1 eq) in i-PrOH (7 mL) was added with Pd(OH)₂ (300.00 mg, 427.24 umol, 20% purity, 8.03e-1 eq) under N₂, and then the suspension was degassed under vacuum and purged with H₂ several times. The mixture was stirred under H₂ (15 psi) at 25° C. for 0.25 h. Upon completion, the combined organic layers were filtered and concentrated under reduced pressure to give (1R,2S,5S)-3-[(2S)-2-amino-3,3-dimethyl-butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (280 mg, crude) as a white solid. MS (ESI) m/z 430.3 [M+H]⁺.

Step 2: (1R,2S,5S)—N-[(1S)-1-cyano-2-[(6R)-5-oxo-4-azaspiro[2.4]heptan-6-yl]ethyl]-3-[(2S)-3,3-dimethyl-2-[(3,3,3-trifluoro-2,2-dimethyl propanoyl)amino]butanoyl]-6,6-dimethyl-3-azabicyclo[3.1.0]hexane-2-carboxamide

A solution of (1R,2S,5S)-3-[(2S)-2-amino-3,3-dimethyl-butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (280 mg, 651.84 umol, 1 eq), 3,3,3-trifluoro-2,2-dimethyl-propanoic acid (101.75 mg, 651.84 umol, 1 eq) in ACN (8 mL) was added 1-methylimidazole (187.31 mg, 2.28 mmol, 181.86 uL, 3.5 eq) [chloro(dimethylamino)methylene]-dimethyl-ammonium; hexafluorophosphate (365.78 mg, 1.30 mmol, 2 eq), and then the mixture stirred at 25° C. for 1 h. Upon completion, the reaction mixture was quenched by addition H₂O 20 mL, and then extracted with EA (30 mL*3). The combined organic layers were washed with brine 50 mL, dried over Na₂SO₄, filtered and concentrated under reduced pressure to give a residue. The residue was purified by prep-HPLC (column: Waters Xbridge Prep OBD C18 150*40 mm*10 um; mobile phase: [water(NH₄HCO₃)-ACN]; B %: 35%-65%, 8 min) to give (1R,2S,5S)—N-[(1S)-1-cyano-2-[(6R)-5-oxo-4-azaspiro[2.4]heptan-6-yl]ethyl]-3-[(2S)-3,3-dimethyl-2-[(3,3,3-trifluoro-2,2-dimethyl-propanoyl)amino]butanoyl]-6,6-dimethyl-3-azabicyclo[3.1.0]hexane-2-carboxamide (20 mg, 35.23 umol, 5.41% yield, 100% purity) as a white solid. MS (ESI) m/z 568.4 [M+H]⁺.

¹H NMR (400 MHz, DMSO-d₆) δ=8.99 (d, J=8.5 Hz, 1H), 7.74 (s, 1H), 7.21 (d, J=9.1 Hz, 1H), 5.00-4.87 (m, 1H), 4.48 (d, J=9.2 Hz, 1H), 4.14 (s, 1H), 3.93-3.82 (m, 1H), 3.68 (d, J=10.6 Hz, 1H), 2.66-2.58 (m, 1H), 2.24-2.15 (m, 1H), 2.02-1.90 (m, 2H), 1.86-1.77 (m, 1H), 1.54 (dd, J=5.5, 7.3 Hz, 1H), 1.35 (d, J=2.1 Hz, 6H), 1.29 (d, J=7.4 Hz, 1H), 1.02 (s, 3H), 0.94 (s, 9H), 0.81 (s, 3H), 0.76-0.70 (m, 1H), 0.59-0.51 (m, 3H)

Example 44: Synthesis of (1R,2S,5S)—N-[(1S)-1-cyan-2-[(6R)-5-oxo-4-azaspiro[2.4]heptan-6-yl]ethyl]-3-[(2S)-3,3-dimethyl-2-[[3,3,3-trifluoro-2-(trifluoromethyl)propanoyl]amino]butanoyl]-6,6-dimethyl-3-azabicyclo[3.1.0]hexane-2-carboxamide (Compound 294)

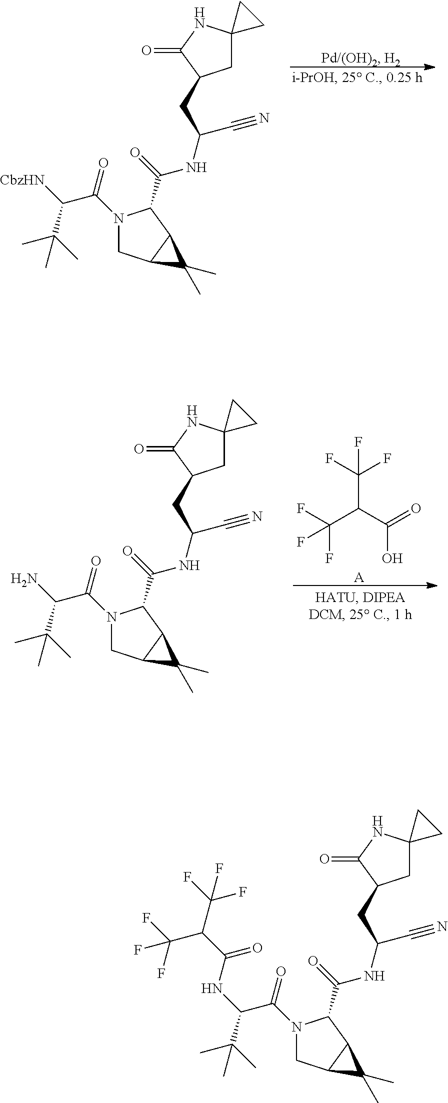

Step 1: (1R,2S,5S)-3-[(2S)-2-amino-3,3-dimethyl-butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide

A solution of benzyl N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl-propyl]carbamate (250 mg, 443.51 umol, 1 eq) in i-PrOH (3 mL) was added Pd(OH)₂ (250.00 mg, 356.04 umol, 20% purity, 8.03e-1 eq) under N₂, and then the suspension was degassed under vacuum and purged with H₂ several times. The mixture was stirred under H₂ (15 psi) at 25° C. for 0.25 h. Upon completion, the organic layer was filtered and concentrated under reduced pressure to give (1R,2S,5S)-3-[(2S)-2-amino-3,3-dimethyl-butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (190 mg, crude) as a white solid. MS (ESI) m/z 430.3 [M+H]⁺.

Step 2: (1R,2S,5S)—N-[(1S)-1-cyano-2-[(6R)-5-oxo-4-azaspiro[2.4]heptan-6-yl]ethyl]-3-[(2S)-3,3-dimethyl-2-[[3,3,3-trifluoro-2-(trifluoromethyl)propanoyl]amino]butanoyl]-6,6-dimethyl-3-azabicyclo[3.1.0]hexane-2-carboxamide

A solution of (1R,2S,5S)-3-[(2S)-2-amino-3,3-dimethyl-butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (190 mg, 442.32 umol, 1 eq), 3,3,3-trifluoro-2-(trifluoromethyl)propanoic acid (95.39 mg, 486.55 umol, 1.1 eq) in DCM (5 mL) was added HATU (336.37 mg, 884.64 umol, 2 eq) and DIPEA (171.50 mg, 1.33 mmol, 231.13 uL, 3 eq). The mixture was stirred at 25° C. for 1 h. Upon completion, the reaction mixture was quenched by addition H₂O 20 mL, and then extracted with DCM (15 mL*3). The combined organic layers were washed with brine 30 mL, dried over Na₂SO₄, filtered and concentrated under reduced pressure to give a residue. The residue was purified by prep-HPLC (column: Phenomenex Luna C18 75*30 mm*3 um; mobile phase: [water (FA)-ACN]; B %: 40% 80%, 8 min) to give (1R,2S,5S)—N-[(1S)-1-cyano-2-[(6R)-5-oxo-4-azaspiro[2.4]heptan-6-yl]ethyl]-3-[(2S)-3,3-dimethyl-2-[[3,3,3-trifluoro-2-(trifluoromethyl)propanoyl]amino]butanoyl]-6,6-dimethyl-3-azabicyclo[3.1.0]hexane-2-carboxamide (27.15 mg, 42.18 umol, 9.54% yield, 94.4% purity) as a white solid. MS (ESI) m/z 608.2 [M+H]⁺.

¹H NMR (400 MHz, DMSO-d₆) δ=9.04 (d, J=8.6 Hz, 1H), 8.84 (br d, J=9.3 Hz, 1H), 7.74 (s, 1H), 5.11-4.88 (m, 2H), 4.48 (d, J=9.4 Hz, 1H), 4.22-4.13 (m, 1H), 3.90 (dd, J=5.4, 10.3 Hz, 1H), 3.71 (br d, J=10.5 Hz, 1H), 2.66-2.61 (m, 1H), 2.20 (ddd, J=4.3, 10.8, 13.5 Hz, 1H), 1.96 (dq, J=9.2, 12.6 Hz, 2H), 1.81 (ddd, J=5.3, 10.9, 13.4 Hz, 1H), 1.55 (dd, J=5.4, 7.5 Hz, 1H), 1.31 (d, J=7.6 Hz, 1H), 1.06-0.91 (m, 12H), 0.83-0.70 (m, 4H), 0.55-0.52 (m, 3H).

Example 45: Synthesis of (1R,2S,5S)-3-[(2S,3R)-3-tert-butoxy-2-[[1-(trifluoromethyl)cyclopropanecarbonyl]amino]butanoyl]-N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (Compound 295)

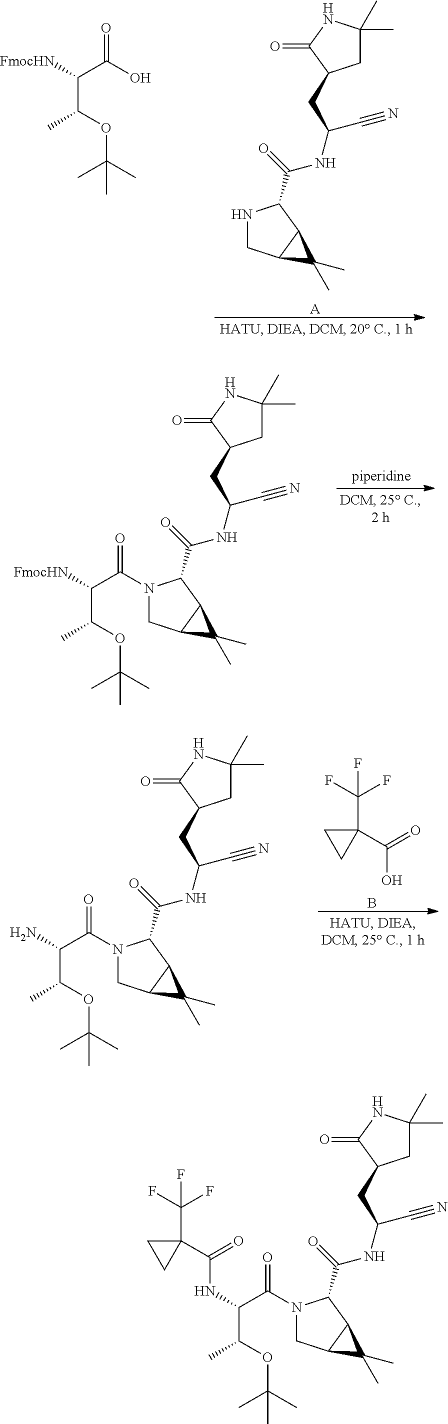

Step 1: 9H-fluoren-9-ylmethyl N-[(1S,2R)-2-tert-butoxy-1-[(1R,2S,5S)-2-[[(1S)-1-cyan-2-[(3R)-5,5-dimethyl-2-oxo pyrrolidin-3-yl]ethyl]carbamoyl]-6,6-dimethyl-3-azabicyclo[3.1.0]hexane-3-carbonyl]propyl]carbamate

To a mixture of (2S,3R)-3-tert-butoxy-2-(9H-fluoren-9-ylmethoxycarbonylamino)butanoic acid (374.48 mg, 942.17 umol, 1 eq) and (1R,2S,5S)—N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (300 mg, 942.17 umol, 1 eq) in DCM (4 mL) was added HATU (537.36 mg, 1.41 mmol, 1.5 eq) and DIEA (243.54 mg, 1.88 mmol, 328.22 uL, 2 eq) in one portion at 20° C. The mixture was stirred at 20° C. for 1 h. Upon completion, the mixture was poured into ice-water (5 mL). The aqueous phase was extracted with DCM (5 mL*3). The combined organic phase was washed with brine (5 mL*3), dried with anhydrous Na₂SO₄, filtered and concentrated in vacuum to get crude product. The crude product was purificated by prep-TLC (SiO₂, Petroleum ether: Ethyl acetate=0:1) to give a 9H-fluoren-9-ylmethyl N-[(1S,2R)-2-tert-butoxy-1-[(1R,2S,5S)-2-[[(1S)-1-cyano-2-[(3R)-5,5-dimethyl-2-oxo-pyrrolidin-3-yl]ethyl]carbamoyl]-6,6-dimethyl-3-azabicyclo[3.1.0]hexane-3-carbonyl]propyl]carbamate (480 mg, 619.03 umol, 65.70% yield, 90% purity) as a white solid. MS (ESI) m/z 698.4 [M+H]⁺.

Step 2: (1R,2S,5S)-3-[(2S,3R)-2-amino-3-tert-butoxy-butanoyl]-N-[(1S)-1-cyano-2-[(3R)-5,5-dimethyl-2-oxo pyrrolidin-3-yl]ethyl]-6,6-dimethyl-3-azabicyclo[3.1.0]hexane-2-carboxamide

To a mixture of 9H-fluoren-9-ylmethyl N-[(1S, 2R)-2-tert-butoxy-1-[(1R,2S,5S)-2-[[(1S)-1-cyano-2-[(3R)-5,5-dimethyl-2-oxo-pyrrolidin-3-yl]ethyl]carbamoyl]-6,6-dimethyl-3-azabicyclo[3.1.0]hexane-3-carbonyl]propyl]carbamate (480 mg, 687.82 umol, 1 eq) in DCM (6 mL) was added piperidine (702.79 mg, 8.25 mmol, 815.11 uL, 12 eq) in one portion at 25° C. The mixture was stirred at 25° C. for 2 h. Upon completion, the mixture was poured into ice-water (5 mL). The aqueous phase was extracted with DCM (5 mL*3). The combined organic phase was washed with brine (5 mL*3), dried with anhydrous Na₂SO₄, filtered and concentrated in vacuum to get crude product. The crude product was purificated by prep-TLC (ethyl acetate: methanol=10:1) to give a (1R,2S,5S)-3-[(2S,3R)-2-amino-3-tert-butoxy-butanoyl]-N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (225.2 mg, 473.48 umol, 68.84% yield) as a white solid. MS (ESI) m/z 476.4 [M+H]⁺.

Step 3: (1R,2S,5S)-3-[(2S,3R)-3-tert-butoxy-2-[[1-(trifluoromethyl)cyclopropanecarbonyl]amino]butanoyl]-N-[(1S)-1-cyan-2-[(3R)-5,5-dimethyl-2-oxo pyrrolidin-3-yl]ethyl]-6,6-dimethyl-3-azabicyclo[3.1.0]hexane-2-carboxamide

To a mixture of (1R,2S,5S)-3-[(2S,3R)-2-amino-3-tert-butoxy-butanoyl]-N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (135 mg, 283.84 umol, 1 eq) and 1-(trifluoromethyl)cyclopropanecarboxylic acid (52.48 mg, 340.61 umol, 1.2 eq) in DCM (1 mL) was added HATU (215.85 mg, 567.68 umol, 2 eq) and DIEA (73.37 mg, 567.68 umol, 98.88 uL, 2 eq) in one portion at 25° C. The mixture was stirred at 25° C. for 2 h. Upon completion, the mixture was poured into ice-water (2 mL). The aqueous phase was extracted with DCM (2 mL*3), combined organic phase was washed with brine (2 mL*3), dried with anhydrous Na₂SO₄, filtered and concentrated in vacuum to get crude product. The crude product was purificated by prep-HPLC (column: Waters Xbridge Prep OBD C18 150*40 mm*10 um; mobile phase: [water (NH₄HCO₃)-ACN]; B %: 35%-65%, 8 min) to give a (1R,2S,5S)-3-[(2S,3R)-3-tert-butoxy-2-[[1-(trifluoromethyl)cyclopropanecarbonyl]amino]butanoyl]-N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (40.2 mg, 65.72 umol, 23.15% yield, 100% purity) as a white solid. MS (ESI) m/z 612.4 [M+H]⁺.

¹H NMR (400 MHz, DMSO-d₆) δ=9.32-8.82 (m, 1H), 7.96-7.71 (m, 1H), 7.57-6.93 (m, 1H), 4.95-4.80 (m, 1H), 4.35-4.17 (m, 1H), 4.12 (s, 1H), 3.94 (dd, J=5.4, 10.4 Hz, 1H), 3.84-3.39 (m, 2H), 2.67 (s, 1H), 2.20-2.06 (m, 1H), 1.98 (dd, J=8.5, 12.1 Hz, 1H), 1.81-1.67 (m, 1H), 1.58-1.40 (m, 2H), 1.34-1.21 (m, 4H), 1.18 (s, 6H), 1.14 1.09 (m, 9H), 1.06 (d, J=6.1 Hz, 2H), 1.02 (s, 3H), 1.01-0.88 (m, 2H), 0.88-0.81 (m, 3H)

Example 46: Synthesis of (1R,2S,5S)—N-[(1S)-1-cyano-2-[(3R)-5,5-dimethyl-2-oxo-pyrrolidin-3-yl]ethyl]-6,6-dimethyl-3-[(2S,3R)-3-phenoxy-2-[(2,2,2-trifluoroacetyl)amino]butanoyl]-3-azabicyclo[3.1.0]hexane-2-carboxamide (Compound 297)

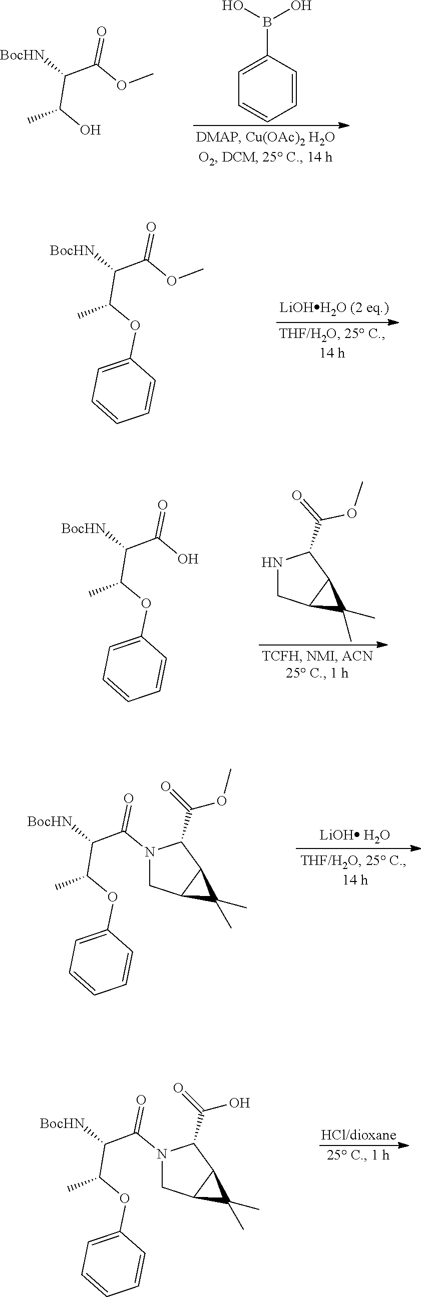

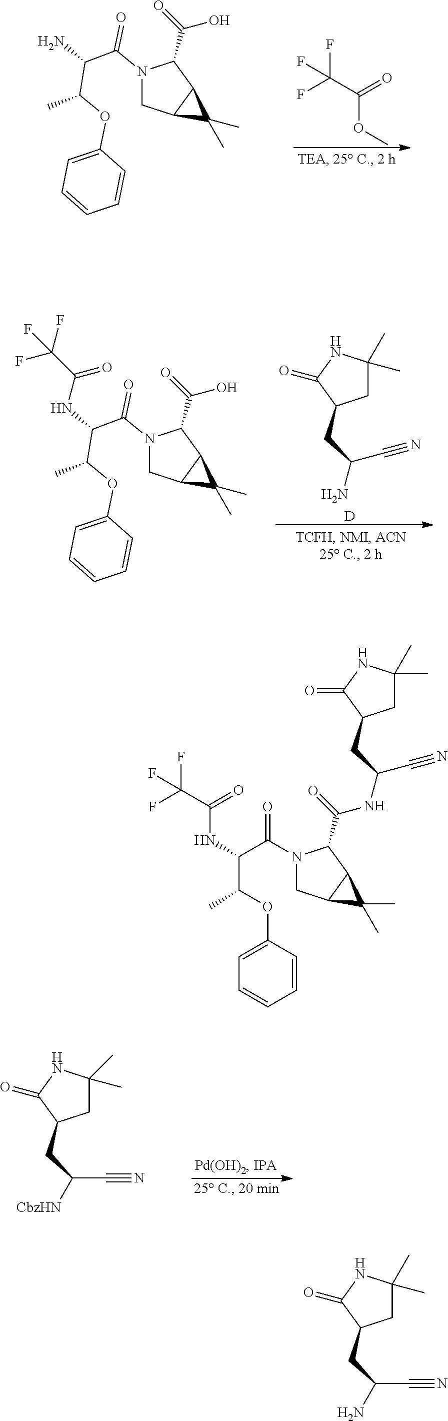

Step 1: (2S)-2-amino-3-[(3R)-5,5-dimethyl-2-oxo pyrrolidin-3-yl]propanenitrile

A solution of benzyl N-[(1S)-1-cyano-2-[(3R)-5,5-dimethyl-2-oxo-pyrrolidin-3-yl]ethyl]carbamate (150 mg, 475.64 umol, 1 eq) in isopropanol (2 mL) was added Pd(OH)₂ (150 mg, 213.62 umol, 20% purity, 0.45 eq) and the mixture was stirred at 25° C. for 20 min under H₂. Upon completion, the reaction was filtered and concentrated in the vacuum to give (2S)-2-amino-3-[(3R)-5,5-dimethyl-2-oxo-pyrrolidin-3-yl]propanenitrile (100 mg, crude) as yellow oil.

Step 2: methyl (2S,3R)-2-(tert-butoxycarbonylamino)-3-phenoxy-butanoate

To a solution of methyl (2S,3R)-2-(tert-butoxycarbonylamino)-3-hydroxy-butanoate (5 g, 21.44 mmol, 1 eq) in DCM (50 mL) was added phenylboronic acid (3.14 g, 25.72 mmol, 1.2 eq) then DMAP (523.74 mg, 4.29 mmol, 0.2 eq) followed by Cu(OAc)₂.H₂O (427.95 mg, 2.14 mmol, 427.95 uL, 0.1 eq) and the mixture was stirred at 25° C. for 14 h under 02 (15 psi). Upon completion, the reaction mixture was quenched by water (100 mL) and extracted with DCM(100 mL*3). The combined organic layers were dried over Na₂SO₄, filtered and concentrated under reduced pressure to give a residue. The residue was purified by column chromatography (SiO₂, Petroleum ether:Ethyl acetate=17:3 to 3:1) to give methyl (2S,3R)-2-(tert-butoxycarbonylamino)-3-phenoxy-butanoate (0.57 g, 1.84 mmol, 4.30% yield) as a yellow oil. MS (ESI) m/z 310.2 [M+H]⁺.

Step 3: (2S,3R)-2-(tert-butoxycarbonylamino)-3-phenoxy-butanoic acid

A solution of methyl (2S,3R)-2-(tert-butoxycarbonylamino)-3-phenoxy-butanoate (450 mg, 1.45 mmol, 1 eq) in THE (5 mL), H₂O (2.5 mL) was added LiOH.H₂O (122.08 mg, 2.91 mmol, 2 eq) and the mixture was stirred at 25° C. for 14 h. Upon completion, the reaction was quenched by HCl (1M, 5 mL) to adjust PH=2, and was extracted with DCM (20 mL*3). The combined organic phase was washed with brine (20 mL), dried with anhydrous Na₂SO₄, filtered and concentrated in vacuum to give (2S,3R)-2-(tert-butoxycarbonylamino)-3-phenoxy-butanoic acid (240 mg, crude) as yellow oil. MS (ESI) m/z 296.2 [M+H]⁺.

Step 4: methyl (1R,2S,5S)-3-[(2S,3R)-2-(tert-butoxycarbonylamino)-3 phenoxy-butanoyl]-6,6-dimethyl-3-azabicyclo[3.1.0]hexane-2-carboxylate

A solution of (2S,3R)-2-(tert-butoxycarbonylamino)-3-phenoxy-butanoic acid (200 mg, 677.21 umol, 1 eq) in ACN (3 mL) was added methyl (1R,2S,5S)-6,6-dimethyl-3-azabicyclo[3.1.0]hexane-2-carboxylate (167.15 mg, 812.65 umol, 1.2 eq, HCl) and [chloro(dimethylamino)methylene]-dimethyl-ammonium; hexafluorophosphate (190.01 mg, 677.21 umol, 1 eq). 1-Methylimidazole (166.80 mg, 2.03 mmol, 161.95 uL, 3 eq) was added and the mixture was stirred at 25° C. for 1 h. Upon completion, the reaction was quenched by H₂O (5 mL), and was extracted with DCM (5 mL*3). The combined organic phase was washed with brine (10 mL), dried with anhydrous Na₂SO₄, filtered and concentrated in vacuum and purified by prep-TLC (SiO₂, PE:EA=3:1) to give methyl (1R,2S,5S)-3-[(2S,3R)-2-(tert-butoxycarbonylamino)-3-phenoxy-butanoyl]-6,6-dimethyl-3-azabicyclo[3.1.0]hexane-2-carboxylate (110 mg, 197.07 umol, 29.10% yield, 80% purity) as a yellow solid. MS (ESI) m/z 447.2 [M+H]⁺.

Step 5: (1R,2S,5S)-3-[(2S,3R)-2-(tert-butoxycarbonylamino)-3 phenoxy-butanoyl]-6,6-dimethyl-3-azabicyclo[3.1.0]hexane-2-carboxylic acid

A solution of methyl (1R,2S,5S)-3-[(2S,3R)-2-(tert-butoxycarbonylamino)-3-phenoxy-butanoyl]-6,6-dimethyl-3-azabicyclo[3.1.0]hexane-2-carboxylate (110 mg, 246.34 umol, 1 eq) in THE (2 mL) and H₂O (1 mL) was added LiOH.H₂O (31.01 mg, 739.02 umol, 3 eq) and the mixture was stirred at 25° C. for 14 h. Upon completion, the reaction was concentrated in the vacuum to give (1R,2S,5S)-3-[(2S,3R)-2-(tert-butoxycarbonylamino)-3-phenoxy-butanoyl]-6,6-dimethyl-3-azabicyclo[3.1.0]hexane-2-carboxylic acid (120 mg, crude) as yellow solid.

Step 6: (1R,2S,5S)-6,6-dimethyl-3-[(2S,3R)-3 phenoxy-2-[(2,2,2-trifluoroacetyl)amino]butanoyl]-3-azabicyclo[3.1.0]hexane-2-carboxylic acid

A solution of (1R,2S,5S)-3-[(2S,3R)-2-(tert-butoxycarbonylamino)-3-phenoxy-butanoyl]-6,6-dimethyl-3-azabicyclo[3.1.0]hexane-2-carboxylic acid (100 mg, 231.21 umol, 1 eq) in HCl/dioxane (4 M, 3 mL, 51.90 eq) was stirred at 25° C. for 1 h. Upon completion, the reaction was concentrated in the vacuum to give (1R,2S,5S)-3-[(2S,3R)-2-amino-3-phenoxy-butanoyl]-6,6-dimethyl-3-azabicyclo[3.1.0]hexane-2-carboxylic acid (100 mg, crude, HCl) as yellow solid. MS (ESI) m/z 333.2 [M+H]⁺.

Step 7: (1R,2S,5S)-6,6-dimethyl-3-[(2S,3R)-3 phenoxy-2-[(2,2,2-trifluoroacetyl)amino]butanoyl]-3-azabicyclo[3.1.0]hexane-2-carboxylic acid

A mixture of (1R,2S,5S)-3-[(2S,3R)-2-amino-3-phenoxy-butanoyl]-6,6-dimethyl-3-azabicyclo[3.1.0]hexane-2-carboxylic acid (100 mg, 300.85 umol, HCl, 1 eq) in methyl 2,2,2-trifluoroacetate (385.24 mg, 3.01 mmol, 303.34 uL, 10 eq) was added TEA (91.33 mg, 902.55 umol, 125.62 uL, 3 eq) and the mixture was stirred at 25° C. for 2 h. Upon completion, the reaction was concentrated in the vacuum to give (1R,2S,5S)-6,6-dimethyl-3-[(2S,3R)-3-phenoxy-2-[(2,2,2-trifluoroacetyl)amino]butanoyl]-3-azabicyclo[3.1.0]hexane-2-carboxylic acid (150 mg, crude) as a yellow solid. MS (ESI) m/z 429.2 [M+H]⁺.

Step 8: (1R,2S,5S)—N-[(1S)-1-cyano-2-[(3R)-5,5-dimethyl-2-oxo pyrrolidin-3-yl]ethyl]-6,6-dimethyl-3-[(2S,3R)-3 phenoxy-2-[(2,2,2-trifluoroacetyl)amino]butanoyl]-3-azabicyclo[3.1.0]hexane-2-carboxamide

A solution of (1R,2S,5S)-6,6-dimethyl-3-[(2S,3R)-3-phenoxy-2-[(2,2,2-trifluoroacetyl)amino]butanoyl]-3-azabicyclo[3.1.0]hexane-2-carboxylic acid (150 mg, 350.14 umol, 1 eq in ACN (3 mL) was added (2S)-2-amino-3-[(3R)-5,5-dimethyl-2-oxo-pyrrolidin-3-yl]propanenitrile (63.46 mg, 350.14 umol, 1 eq), then 1-methylimidazole (86.24 mg, 1.05 mmol, 83.73 uL, 3 eq). Then, TCFH (98.24 mg, 350.14 umol, 1 eq) was added and the mixture was stirred at 25° C. for 2 h. Upon completion, the reaction was quenched by H₂O (5 mL), and was extracted with DCM (5 mL*3). The combined organic phase was washed with brine (20 mL), dried with anhydrous Na₂SO₄, filtered and concentrated in vacuum and purified by prep-HPLC (column: Phenomenex C18 80*40 mm*3 um; mobile phase: [water(NH₄HCO₃)-ACN]; B %: 30%-50%, 8 min) to give (1R,2S,5S)—N-[(1S)-1-cyano-2-[(3R)-5,5-dimethyl-2-oxo-pyrrolidin-3-yl]ethyl]-6,6-dimethyl-3-[(2S,3R)-3-phenoxy-2-[(2,2,2-trifluoroacetyl)amino]butanoyl]-3-azabicyclo[3.1.0]hexane-2-carboxamide (8.43 mg, 14.25 umol, 4.07% yield, 100% purity) as a white solid. MS (ESI) m/z 592.2 [M+H]⁺.

¹H NMR (400 MHz, DMSO-d₆) δ=10.15 (br s, 1H), 9.04 (d, J=8.5 Hz, 1H), 7.83 (s, 1H), 7.27 (t, J=7.9 Hz, 2H), 7.06-6.78 (m, 3H), 5.03-4.85 (m, 1H), 4.67 (br d, J=2.6 Hz, 2H), 4.15 (s, 1H), 4.03-3.99 (m, 1H), 3.86 (br d, J=10.4 Hz, 1H), 2.63-2.55 (m, 1H), 2.20-2.10 (m, 1H), 2.05-1.95 (m, 1H), 1.77-1.70 (m, 1H), 1.64-1.48 (m, 2H), 1.35 (d, J=7.6 Hz, 1H), 1.30-1.25 (m, 3H), 1.17 (d, J=16.8 Hz, 6H), 1.04 (s, 3H), 0.92-0.84 (m, 3H).

Example 47: Synthesis of (1R,2S,5S)—N-[(1S)-1-cyano-2-[(3R)-5,5-dimethyl-2-oxo-pyrrolidin-3-yl]ethyl]-3-[(2S,3R)-3-(cyclopropoxy)-2-[(2,2,2-trifluoroacetyl)amino]butanoyl]-6,6-dimethyl-3-azabicyclo[3.1.0]hexane-2-carboxamide (Compound 298) and (1R,2S,5S)—N-[(1S)-1-cyan-2-[(3R)-5,5-dimethyl-2-oxo-pyrrolidin-3-yl]ethyl]-3-[(2R,3R)-3-(cyclopropoxy)-2-[(2,2,2-trifluoroacetyl)amino]butanoyl]-6,6-dimethyl-3-azabicyclo[3.1.0]hexane-2-carboxamide (Compound 298a)

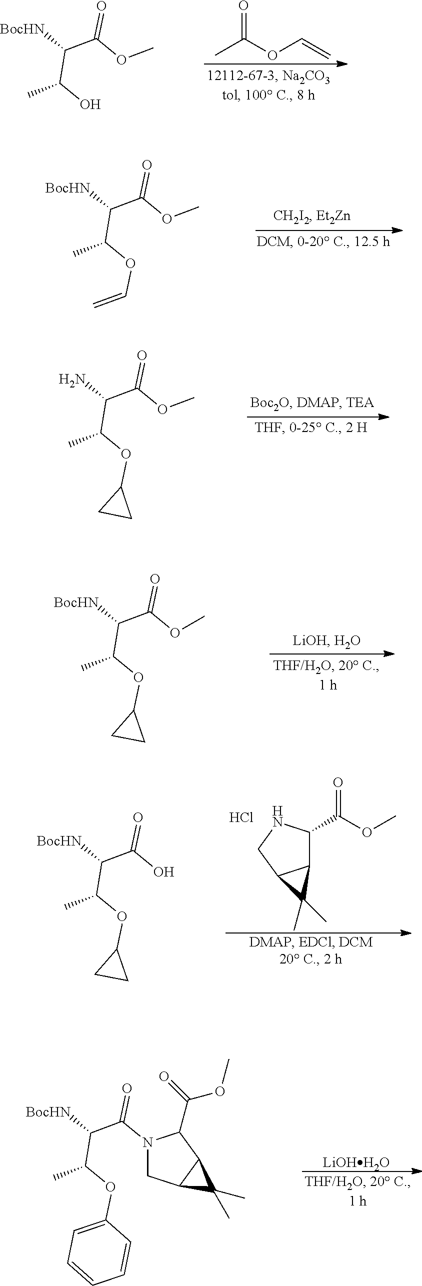

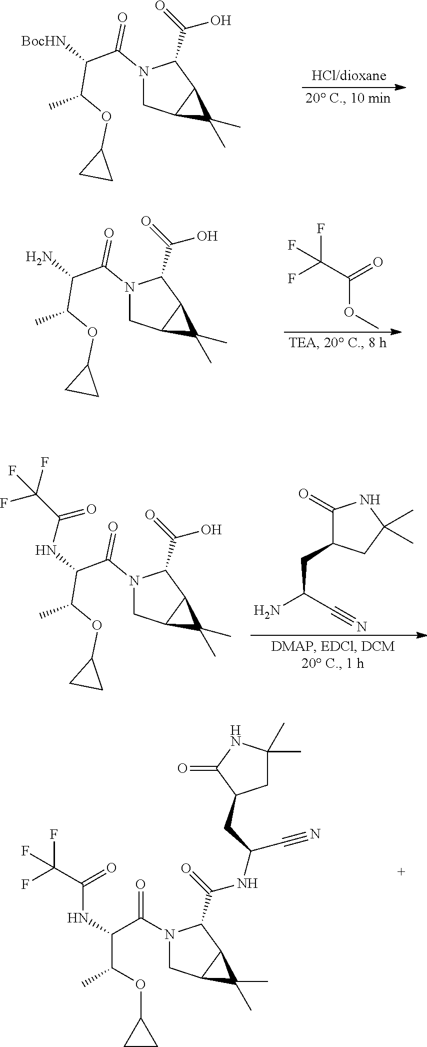

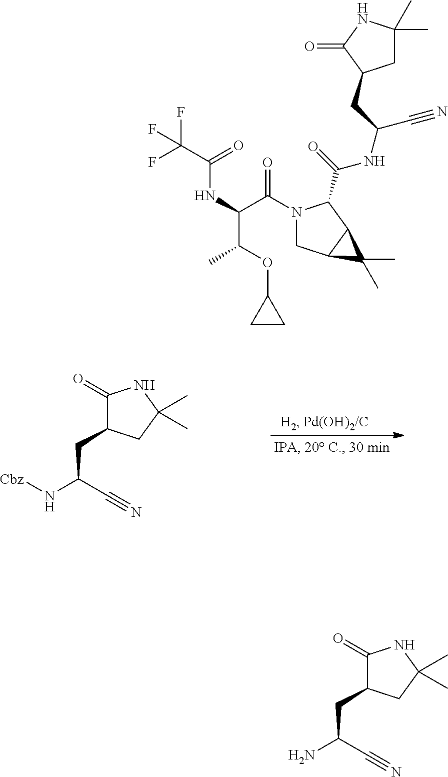 [SMILES: C[C@@H](OC1CC1)[C@@H](NC(=O)C(F)(F)F)C(=O)N1C[C@H]2[C@@H]([C@H]1C(=O)N[C@H](C#N)C[C@@H]1CC(C)(C)NC1=O)C2(C)C ; CC1(C)C[C@@H](C[C@@H](C#N)NC(=O)OCc2ccccc2)C(=O)N1 ; CC1(C)C[C@@H](C[C@H](N)C#N)C(=O)N1]

Step 1: (2S)-2-amino-3-[(3R)-5,5-dimethyl-2-oxo pyrrolidin-3-yl]propanenitrile

To a solution of benzyl N-[(1S)-1-cyano-2-[(3R)-5,5-dimethyl-2-oxo-pyrrolidin-3-yl]ethyl]carbamate (0.3 g, 951.28 umol, 1 eq) in IPA (3 mL) was added Pd(OH)₂/C (0.2 g, 20% purity) under N₂ atmosphere. The suspension was degassed and purged with H₂ for 3 times. The mixture was stirred under H₂ (15 Psi) at 20° C. for 30 min. The reaction mixture was filtered to get the filtrate. Then the filtrate was concentrated to get the residue. (2S)-2-amino-3-[(3R)-5,5-dimethyl-2-oxo-pyrrolidin-3-yl]propanenitrile (150 mg, 827.66 umol, 87.01% yield) was obtained as a colorless oil and used directly next step. MS (ESI) m/z 182.1 [M+H]⁺.

Step 2: methyl (2S,3R)-2-(tert-butoxycarbonylamino)-3-vinyloxy-butanoate

To a solution of methyl (2S,3R)-2-(tert-butoxycarbonylamino)-3-hydroxy-butanoate (2.6 g, 11.15 mmol, 1 eq) and vinyl acetate (1.92 g, 22.29 mmol, 2.06 mL, 2 eq) in toluene (20 mL) was added chloroiridium; (1Z, 5 Z)-cycloocta-1,5-diene (112.31 mg, 167.19 umol, 0.015 eq) and Na₂CO₃ (1 g, 9.47 mmol, 0.85 eq) at 20° C. The mixture was stirred at 100° C. for 8 h. Upon completion, the reaction mixture was cooled to 25° C. and concentrated to get the residue. The residue was purified by column chromatography (SiO₂, Petroleum ether:Ethyl acetate=50:1 to 10:1). Methyl (2S, 3R)-2-(tert-butoxycarbonylamino)-3-vinyloxy-butanoate (2.5 g, 9.64 mmol, 86.50% yield) was obtained as a yellow oil. MS (ESI) m/z 260.1 [M+H]⁺.

¹H NMR (400 MHz, DMSO-d₆) δ=7.05 (br d, J=8.9 Hz, 1H), 6.45-6.22 (m, 1H), 4.42-4.29 (m, 1H), 4.28-4.19 (m, 2H), 4.01-3.94 (m, 1H), 3.64 (s, 3H), 1.39 (s, 9H), 1.14(d, J=6.2 Hz, 3H).

Step 3: methyl (2S,3R)-2-amino-3-(cyclopropoxy)butanoate

Under argon, ZnEt2 (1 M, 9.64 mL, 2.5 eq) in hexane were initially charged in DCM (20 mL). CH₂I2 (2.54 g, 9.49 mmol, 765.36 uL, 2.46 eq) in DCM (8 mL) were then added dropwise at 0° C. After 30 min, methyl (2S,3R)-2-(tert-butoxycarbonylamino)-3-vinyloxy-butanoate (1 g, 3.86 mmol, 1 eq) in DCM (20 mL) were added dropwise and the reaction mixture was warmed to 20° C. and stirred 12 h. Upon completion, the reaction mixture was quenched by water (0.5 mL) and concentrated to get residue. The residue was triturated and filtered to get the filtate and concentrated to get the product. Methyl (2S,3R)-2-amino-3-(cyclopropoxy)butanoate (0.85 g, crude) was obtained as a light yellow oil. MS (ESI) m/z 174.1 [M+H]⁺.

Step 4: methyl (2S,3R)-2-(tert-butoxycarbonylamino)-3-(cyclopropoxy)butanoate

To a solution of methyl (2S,3R)-2-amino-3-(cyclopropoxy)butanoate (0.8 g, 4.62 mmol, 1 eq) in THE (6 mL) was added DMAP (282.13 mg, 2.31 mmol, 0.5 eq), TEA (934.72 mg, 9.24 mmol, 1.29 mL, 2 eq), and Boc₂O (1.01 g, 4.62 mmol, 1.06 mL, 1 eq). The mixture was stirred at 20° C. for 2 h. Upon completion, the reaction mixture was added water (10 mL) and extracted with ethyl acetate (10 mL*2) to get the organic phase. The combined organic layers were washed with brine (10 mL), dried over anhydrous sodium sulfate, filtered and concentrated under reduced pressure to give a residue. The residue was purified by column chromatography (SiO₂, Petroleum ether:Ethyl acetate=1:1). Methyl (2S,3R)-2-(tert-butoxycarbonylamino)-3-(cyclopropoxy)butanoate (0.65 g, 2.38 mmol, 51.49% yield) was obtained as a light yellow oil. MS (ESI) m/z 274.2 [M+H]⁺.

¹H NMR (400 MHz, DMSO-d₆) S=6.69 (brd, J=8.9 Hz, 1H), 4.18-4.11 (m, 1H), 3.99-3.90 (m, 1H), 3.63 (s, 3H), 3.34-3.24 (m, 1H), 1.38 (s, 9H), 1.13 (d, J=6.4 Hz, 3H), 0.48-0.28 (m, 4H)

Step 5: (2S,3R)-2-(tert-butoxycarbonylamino)-3-(cyclopropoxy)butanoic acid

To a solution of methyl (2S,3R)-2-(tert-butoxycarbonylamino)-3-(cyclopropoxy)butanoate (650 mg, 2.38 mmol, 1 eq) in THE (10 mL) and H₂O (3 mL) was added LiOH.H₂O (299.38 mg, 7.13 mmol, 3 eq). The mixture was stirred at 20° C. for 1 h. Upon completion, the reaction mixture was added 1 N HCl to pH-6 and extracted with ethyl acetate (10 mL*2). The organic phase was washed with brine (5 mL), dried over anhydrous magnesium sulfate, filtered, and concentrated under reduced pressure. (2S,3R)-2-(tert-butoxycarbonylamino)-3-(cyclopropoxy)butanoic acid (0.6 g, crude) was obtianed as a white solid and used directly next step. MS (ESI) m/z 260.1 [M+H]⁺.

¹H NMR (400 MHz, DMSO-d₆) δ=12.62 (br s, 1H), 6.32 (br d, J=9.2 Hz, 1H), 4.02-3.99 (m, 1H), 3.99-3.94 (m, 1H), 3.39-3.21 (m, 2H), 1.38 (s, 9H), 1.14 (d, J=6.2 Hz, 2H), 0.52-0.29 (m, 4H)

Step 6: methyl (1R,2S,5S)-3-[(2S,3R)-2-(tert-butoxycarbonylamino)-3-(cyclopropoxy)butanoyl]-6,6-dimethyl-3-azabicyclo[3.1.0]hexane-2-carboxylate

To a solution of (2S,3R)-2-(tert-butoxycarbonylamino)-3-(cyclopropoxy)butanoic acid (0.5 g, 1.93 mmol, 1 eq) and methyl (1R,2S,5S)-6,6-dimethyl-3-azabicyclo[3.1.0]hexane-2-carboxylate (475.93 mg, 2.31 mmol, 1.2 eq, HCl) in DCM (5 mL) was added DMAP (706.74 mg, 5.78 mmol, 3 eq) and EDCI (739.30 mg, 3.86 mmol, 2 eq). The mixture was stirred at 20° C. for 2 h. Upon completion, the reaction mixture was added water (10 mL) and expected with DCM (10 mL*2) to get the organic phase. The combined organic layers were washed with brine (10 mL), dried over anhydrous sodium sulfate, filtered and concentrated under reduced pressure to give a residue. The residue was purified by column chromatography (SiO₂, Petroleum ether:Ethyl acetate=1:1). Methyl (1R,2S,5S)-3-[(2S,3R)-2-(tert-butoxycarbonylamino)-3-(cyclopropoxy)butanoyl]-6,6-dimethyl-3-azabicyclo[3.1.0]hexane-2-carboxylate (580 mg, 1.36 mmol, 70.34% yield, 96% purity) was obtained as a colorless oil. MS (ESI) m/z 411.2 [M+H]⁺.

Step 7: (1R,2S,5S)-3-[(3R)-2-(tert-butoxycarbonylamino)-3-(cyclopropoxy)butanoyl]-6,6-dimethyl-3-azabicyclo[3.1.0]hexane-2-carboxylic acid

To a solution of methyl (1R,2S,5S)-3-[(3R)-2-(tert-butoxycarbonylamino)-3-(cyclopropoxy)butanoyl]-6,6-dimethyl-3-azabicyclo[3.1.0]hexane-2-carboxylate (0.2 g, 487.21 umol, 1 eq) in THE (3 mL) and H₂O (1 mL) was added LiOH.H₂O (61.33 mg, 1.46 mmol, 3 eq). The mixture was stirred at 20° C. for 1 h. Upon completion, the reaction mixture was added 1 N HCl to pH-4 and expected with ethyl acetate (10 mL*2). The organic phase was washed with brine (5 mL), dried over anhydrous magnesium sulfate, filtered, concentrated under reduced pressure. (1R,2S,5S)-3-[(3R)-2-(tert-butoxycarbonylamino)-3-(cyclopropoxy)butanoyl]-6,6-dimethyl-3-azabicyclo[3.1.0]hexane-2-carboxylic acid (158 mg, 398.51 umol, 81.79% yield) was obtained as a white solid and used directly next step. MS (ESI) m/z 397.2 [M+H]⁺.

Step 8: (1R,2S,5S)-3-[(2S,3R)-2-amino-3-(cyclopropoxy)butanoyl]-6,6-dimethyl-3-azabicyclo[3.1.0]hexane-2-carboxylic acid

To a solution of (1R,2S,5S)-3-[(3R)-2-(tert-butoxycarbonylamino)-3-(cyclopropoxy)butanoyl]-6,6-dimethyl-3-azabicyclo[3.1.0]hexane-2-carboxylic acid (158 mg, 398.51 umol, 1 eq) was added HCl/dioxane (4 M, 996.28 uL, 10 eq). The mixture was stirred at 20° C. for 10 min. The reaction mixture was concentrated to get the residue. (1R,2S,5S)-3-[(2S,3R)-2-amino-3-(cyclopropoxy)butanoyl]-6,6-dimethyl-3-azabicyclo[3.1.0]hexane-2-carboxylic acid (125 mg, crude, HCl) was obtained as a colorless oil and used directly next step. MS (ESI) m/z 297.2 [M+H]⁺.

Step 9: (1R,2S,5S)-3-[(2S,3R)-3-(cyclopropoxy)-2-[(2,2,2-trifluoroacetyl)amino]butanoyl]-6,6-dimethyl-3-azabicyclo[3.1.0]hexane-2-carboxylic acid

To a solution of (1R,2S,5S)-3-[(2S,3R)-2-amino-3-(cyclopropoxy)butanoyl]-6,6-dimethyl-3-azabicyclo[3.1.0]hexane-2-carboxylic acid (125 mg, 421.78 umol, 1 eq) in methyl 2,2,2-trifluoroacetate (3 mL) was added TEA (128.04 mg, 1.27 mmol, 176.12 uL, 3 eq). The mixture was stirred at 20° C. for 8 h. The reaction mixture was concentrated and added 1 N HCl to adjust pH to about 4 and extracted with ethyl acetate (10 mL*2). The organic phase was washed with brine (5 mL), dried over anhydrous magnesium sulfate, filtered, concentrated under reduced pressure. (1R,2S,5S)-3-[(2S,3R)-3-(cyclopropoxy)-2-[(2,2,2-trifluoroacetyl)amino]butanoyl]-6,6-dimethyl-3-azabicyclo[3.1.0]hexane-2-carboxylic acid (150 mg, crude) was obtained as a light yellow oil and used directly for next step. MS (ESI) m/z 393.2 [M+H]⁺.

Step 10: (1R,2S,5S)—N-[(1S)-1-cyano-2-[(3R)-5,5-dimethyl-2-oxo pyrrolidin-3-yl]ethyl]-3-[(2S,3R)-3-(cyclopropoxy)-2-[(2,2,2-trifluoroacetyl)amino]butanoyl]-6,6-dimethyl-3-azabicyclo[3.1.0]hexane-2-carboxamide (Compound 298) and (1R,2S,5S)—N-[(1S)-1-cyano-2-[(3R)-5,5-dimethyl-2-oxo pyrrolidin-3-yl]ethyl]-3-[(2R,3R)-3-(cyclopropoxy)-2-[(2,2,2-trifluoroacetyl)amino]butanoyl]-6,6-dimethyl-3-azabicyclo[3.1.0]hexane-2-carboxamide (Compound 298a)

To a solution of (1R,2S,5S)-3-[(2S,3R)-3-(cyclopropoxy)-2-[(2,2,2-trifluoroacetyl)amino]butanoyl]-6,6-dimethyl-3-azabicyclo[3.1.0]hexane-2-carboxylic acid (130 mg, 331.32 umol, 1 eq), (2S)-2-amino-3-[(3R)-5,5-dimethyl-2-oxo-pyrrolidin-3-yl]propanenitrile (90.07 mg, 496.98 umol, 1.5 eq) in ACN (0.5 mL) was added 1-[chloro(pyrrolidin-1-ium-1-ylidene)methyl]pyrrolidine; hexafluorophosphate (220.43 mg, 662.64 umol, 2 eq) and 1-methylimidazole (81.61 mg, 993.96 umol, 79.23 uL, 3 eq). The mixture was stirred at 20° C. for 1 h. The reaction mixture was added water (5 mL) and extracted with ethyl acetate (5 mL*2). The organic phase was washed with brine (5 mL), dried over anhydrous magnesium sulfate, filtered, concentrated under reduced pressure. The residue was purified by prep-HPLC (column: Phenomenex C₁₋₈ 75*30 mm*3 um; mobile phase: [water (NH₄HCO₃)-ACN]; B %: 25%-55%, 8 min). (1R,2S,5S)—N-[(1S)-1-cyano-2-[(3R)-5,5-dimethyl-2-oxo-pyrrolidin-3-yl]ethyl]-3-[(2S,3R)-3-(cyclopropoxy)-2-[(2,2,2-trifluoroacetyl)amino]butanoyl]-6,6-dimethyl-3-azabicyclo[3.1.0]hexane-2-carboxamide (Compound 298) (72 mg, 125.89 umol, 38.00% yield, 97.14% purity) was obtained as a white solid. MS (ESI) m/z 556.3 [M+H]⁺.

¹H NMR (400 MHz, DMSO-d₆) δ=9.85 (br s, 1H), 9.01 (d, J=8.5 Hz, 1H), 7.87-7.74 (m, 1H), 4.98-4.87 (m, 1H), 4.44-4.29 (m, 1H), 4.14-4.07 (m, 1H), 3.97-3.88 (m, 1H), 3.85-3.73 (m, 2H), 3.38-3.33 (m, 1H), 2.61-2.54 (m, 1H), 2.20-2.10 (m, 1H), 2.01-1.93 (m, 1H), 1.77-1.67 (m, 1H), 1.60-1.47 (m, 2H), 1.33 (d, J=7.6 Hz, 1H), 1.24 1.16 (m, 6H), 1.15-1.10 (m, 3H), 1.03 (s, 3H), 0.85 (s, 3H), 0.60-0.52 (m, 1H), 0.47-0.36 (m, 2H), 0.35-0.26 (m, 1H).

(1R,2S,5S)—N-[(1S)-1-cyano-2-[(3R)-5,5-dimethyl-2-oxo-pyrrolidin-3-yl]ethyl]-3-[(2R,3R)-3-(cyclopropoxy)-2-[(2,2,2-trifluoroacetyl)amino]butanoyl]-6,6-dimethyl-3-azabicyclo[3.1.0]hexane-2-carboxamide (Compound 298a) (2.39 mg, 4.30 umol, 1.30% yield, 100% purity) was obtained as a white solid. MS (ESI) m/z 556.3 [M+H]⁺.

¹H NMR (400 MHz, DMSO-d₆) δ=9.91-9.67 (m, 1H), 8.85-8.62 (m, 1H), 8.04-7.63 (m, 1H), 4.99-4.85 (m, 1H), 4.56-4.45 (m, 1H), 4.06 (s, 1H), 3.99-3.75 (m, 3H), 3.57 (br s, 1H), 2.63-2.58 (m, 1H), 2.15-2.09 (m, 1H), 2.03-1.97 (m, 1H), 1.78-1.74 (m, 1H), 1.59-1.54 (m, 2H), 1.29 (br s, 1H), 1.22-1.15 (m, 6H), 1.10 (br s, 3H), 1.03 (s, 3H), 0.94 (s, 3H), 0.62-0.53 (m, 1H), 0.49-0.41 (m, 2H), 0.31 (br s, 1H)

Example 48: Synthesis of (1R,2S,5S)—N-[(1S)-1-cyano-2-[(3R)-5,5-dimethyl-2-oxo-pyrrolidin-3-yl]ethyl]-6,6-dimethyl-3-[(2S,3R)-3-(1-methylcyclopropoxy)-2-[(2,2,2-trifluoroacetyl)amino]butanoyl]-3-azabicyclo[3.1.0]hexane-2-carboxamide (Compound 299) and (1R,2R,5S)—N-[(1S)-1-cyano-2-[(3R)-5,5-dimethyl-2-oxo-pyrrolidin-3-yl]ethyl]-6,6-dimethyl-3-[(2S,3R)-3-(1-methylcyclopropoxy)-2-[(2,2,2-trifluoroacetyl)amino]butanoyl]-3-azabicyclo[3.1.0]hexane-2-carboxamide (Compound 299a)

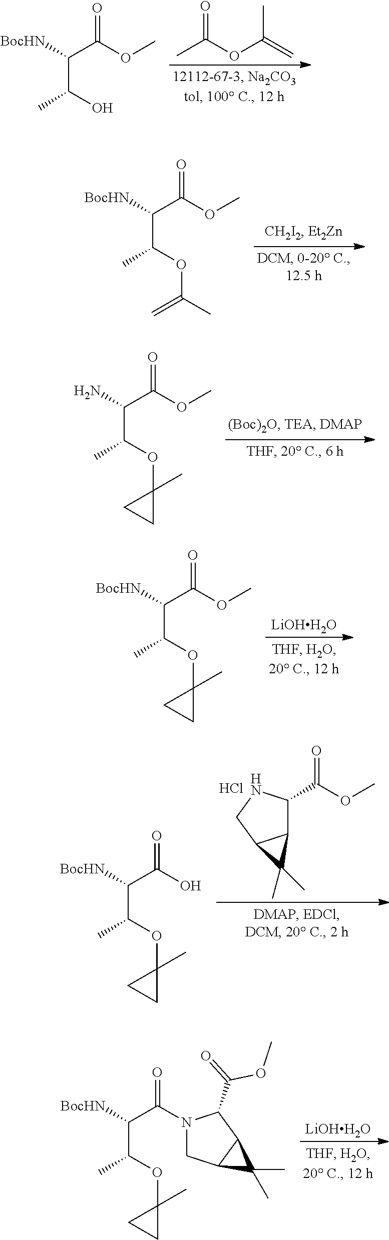

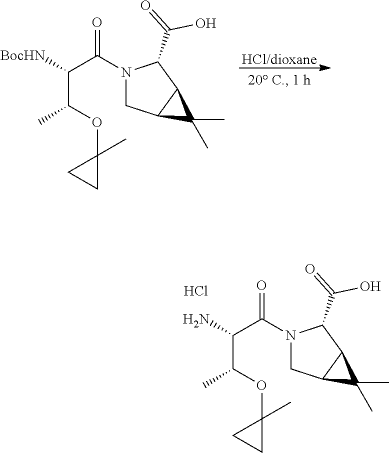 [SMILES: C[C@@H](OC1(C)CC1)[C@H](NC(=O)OC(C)(C)C)C(=O)N1C[C@H]2[C@@H]([C@H]1C(=O)O)C2(C)C ; "HCl/dioxane" ; Cl ; C[C@@H](OC1(C)CC1)[C@H](N)C(=O)N1C[C@H]2[C@@H]([C@H]1C(=O)O)C2(C)C]

Step 1: (2S)-2-amino-3-[(3R)-5,5-dimethyl-2-oxo pyrrolidin-3-yl]propanenitrile

To a solution of benzyl N-[(1S)-1-cyano-2-[(3R)-5,5-dimethyl-2-oxo-pyrrolidin-3-yl]ethyl]carbamate (400 mg, 1.27 mmol, 1 eq) in isopropanol (5 mL) was added Pd(OH)₂/C (561.11 mg, 20% purity) under N₂ atmosphere. The suspension was degassed and purged with H₂ for 3 times. The mixture was stirred under H₂ (15 Psi) at 20° C. for 1 h. The reaction mixture was filtered to get the filtrate. Then the filtrate was concentrated to get the residue. (2S)-2-amino-3-[(3R)-5,5-dimethyl-2-oxo-pyrrolidin-3-yl]propanenitrile (150 mg, 827.66 umol, 87.01% yield) was obtained as a colorless oil and used directly for next step. MS (ESI) m/z 182.1 [M+H]⁺.

Step 2: methyl (2S,3R)-2-(tert-butoxycarbonylamino)-3-isopropenyloxy-butanoate

To a solution of methyl (2S,3R)-2-(tert-butoxycarbonylamino)-3-hydroxy-butanoate (20 g, 85.74 mmol, 1 eq) and isopropenyl acetate (8.58 g, 85.74 mmol, 9.33 mL, 1 eq) in toluene (300 mL) was added chloroiridium; (1Z,5Z)-cycloocta-1,5-diene (863.89 mg, 1.29 mmol, 0.015 eq) and Na₂CO₃ (9.09 g, 85.74 mmol, 1 eq) at 20° C. The mixture was stirred at 100° C. for 12 h. Upon completion, the reaction mixture was cooled to 25° C. and concentrated to get the residue. The residue was purified by column chromatography (SiO₂, Petroleum ether: Ethyl acetate=50:1 to 20:1) to give methyl (2S,3R)-2-(tert-butoxycarbonylamino)-3-isopropenyloxy-butanoate (7.2 g, 26.34 mmol, 30.72% yield) as a brown liquid. MS (ESI) m/z 274.2 [M+H]⁺.

Step 3: methyl (2S,3R)-2-amino-3-(1-methylcyclopropoxy)butanoate

Under argon, ZnEt₂ (1 M, 18.29 mL, 2.5 eq) in hexane were initially charged in DCM (20 mL) and then CH₂I₂ (4.82 g, 18.00 mmol, 1.45 mL, 2.46 eq) in DCM (10 mL) were added dropwise at 0° C. After 30 mins, methyl (2S,3R)-2-(tert-butoxycarbonylamino)-3-isopropenyloxy-butanoate (2 g, 7.32 mmol, 1 eq) in DCM (20 mL) were added dropwise and the reaction mixture was warmed to 20° C. and stirred 12 h. Upon completion, the reaction mixture was added with water (1 mL) and concentrated to get residue. The residue was triturated with ethyltrate (10 mL) and filtered to get the filtate then concentrated to give methyl (2S, 3R)-2-amino-3-(1-methylcyclopropoxy) butanoate (800 mg, crude) as a yellow liquid. MS (ESI) m/z 188.1 [M+H]⁺.

Step 4: methyl (2S,3R)-2-(tert-butoxycarbonylamino)-3-(1-methylcyclopropoxy)butanoate

To a solution of methyl (2S,3R)-2-amino-3-(1-methylcyclopropoxy)butanoate (500 mg, 2.67 mmol, 1 eq) in THE (10 mL) was added DMAP (163.12 mg, 1.34 mmol, 0.5 eq), TEA (540.44 mg, 5.34 mmol, 743.38 uL, 2 eq) and (Boc)₂O (582.81 mg, 2.67 mmol, 613.49 uL, 1 eq). The mixture was stirred at 20° C. for 6 h. Upon completion, the reaction mixture was added water (10 mL) and extracted with ethyl acetate (10 mL*2) to get the organic phase. The combined organic layers were washed with brine (10 mL), dried over anhydrous sodium sulfate, filtered and concentrated under reduced pressure to give a residue. The residue was purified by column chromatography (SiO₂, Petroleum ether: Ethyl acetate=50:1 to 10:1). Methyl (2S,3R)-2-(tert-butoxycarbonylamino)-3-(1-methylcyclopropoxy)butanoate (750 mg, 2.61 mmol, 97.74% yield) was obtained as a light yellow oil. MS (ESI) m/z 288.2 [M+H]⁺.

Step 5: (2S,3R)-2-(tert-butoxycarbonylamino)-3-(1-methylcyclopropoxy)butanoic acid

To a solution of methyl (2S,3R)-2-(tert-butoxycarbonylamino)-3-(1-methylcyclopropoxy)butanoate (0.75 g, 2.61 mmol, 1 eq) in THE (9 mL), H₂O (3 mL) was added LiOH.H₂O (328.55 mg, 7.83 mmol, 3 eq). The mixture was stirred at 20° C. for 12 h. Upon completion, the reaction mixture was added 1 N HCl to pH about 6 and extracted with ethyl acetate (10 mL*2). The organic phase was washed with brine (5 mL), dried over anhydrous magnesium sulfate, filtered, concentrated under reduced pressure. (2S,3R)-2-(tert-butoxycarbonylamino)-3-(1-methylcyclopropoxy)butanoic acid (730 mg, crude) as a white solid and used directly next step. MS (ESI) m/z 274.2 [M+H]⁺.

Step 6: methyl (1R,2S,5S)-3-[(2S,3R)-2-(tert-butoxycarbonylamino)-3-(1-methylcyclopropoxy)butanoyl]-6,6-dimethyl-3-azabicyclo[3.1.0]hexane-2-carboxylate

To a solution of (2S,3R)-2-(tert-butoxycarbonylamino)-3-(1-methylcyclopropoxy)butanoic acid (630 mg, 2.30 mmol, 1 eq), methyl (1R,2S,5S)-6,6-dimethyl-3-azabicyclo[3.1.0]hexane-2-carboxylate (474.09 mg, 2.30 mmol, 1 eq, HCl) in DCM (10 mL) was added DMAP (844.79 mg, 6.91 mmol, 3 eq) and EDCI (883.72 mg, 4.61 mmol, 2 eq). The mixture was stirred at 20° C. for 2 h. Upon completion, the reaction mixture was added with water (10 mL) and extracted with DCM (10 mL*2) to get the organic phase. The combined organic layers were washed with brine (10 mL), dried over anhydrous sodium sulfate, filtered and concentrated under reduced pressure to give a residue. The residue was purified by column chromatography (SiO₂, Petroleum ether: Ethyl acetate=30:1 to 5:1). Methyl (1R,2S,5S)-3-[(2S,3R)-2-(tert-butoxycarbonylamino)-3-(1-methylcyclopropoxy)butanoyl]-6,6-dimethyl-3-azabicyclo[3.1.0]hexane-2-carboxylate (530 mg, 1.05 mmol, 45.50% yield, 84% purity) was obtained as a yellow oil. MS (ESI) m/z 425.3 [M+H]⁺

Step 7: (1R,2S,5S)-3-[(2S,3R)-2-(tert-butoxycarbonylamino)-3-(1-methylcyclopropoxy)butanoyl]-6,6-dimethyl-3-azabicyclo[3.1.0]hexane-2-carboxylic acid

To a solution of methyl (1R,2S,5S)-3-[(2S,3R)-2-(tert-butoxycarbonylamino)-3-(1-methylcyclopropoxy)butanoyl]-6,6-dimethyl-3-azabicyclo[3.1.0]hexane-2-carboxylate (300 mg, 706.66 umol, 1 eq) in THE (3 mL) and H₂O (1 mL) was added LiOH.H₂O (88.95 mg, 2.12 mmol, 3 eq). The mixture was stirred at 20° C. for 12 h. Upon completion, the reaction mixture was added 1 N HCl to pH-4 and extracted with ethyl acetate (10 mL*2). The organic phase was washed with brine (5 mL), dried over anhydrous magnesium sulfate, filtered, concentrated under reduced pressure to give (1R,2S,5S)-3-[(2S,3R)-2-(tert-butoxycarbonylamino)-3-(1-methylcyclopropoxy)butanoyl]-6,6-dimethyl-3-azabicyclo[3.1.0]hexane-2-carboxylic acid (260 mg, crude) as a white solid and used directly for next step. MS (ESI) m/z 411.2 [M+H]⁺.

Step 8: (1R,2S,5S)-3-[(2S,3R)-2-amino-3-(1-methylcyclopropoxy)butanoyl]-6,6-dimethyl-3-azabicyclo[3.1.0]hexane-2-carboxylic acid

A solution of (1R,2S,5S)-3-[(2S,3R)-2-(tert-butoxycarbonylamino)-3-(1-methylcyclopropoxy)butanoyl]-6,6-dimethyl-3-azabicyclo[3.1.0]hexane-2-carboxylic acid (260 mg, 633.37 umol, 1 eq) in HCl/dioxane (4 M, 3 mL, 18.95 eq) was stirred at 20° C. for 1 h. Upon completion, the reaction mixture was concentrated to give (1R,2S,5S)-3-[(2S,3R)-2-amino-3-(1-methylcyclopropoxy)butanoyl]-6,6-dimethyl-3-azabicyclo[3.1.0]hexane-2-carboxylic acid (180 mg, crude, HCl) as a white solid and used directly next step. MS (ESI) m/z 311.2 [M+H]⁺.

Step 9: (1R,2S,5S)-6,6-dimethyl-3-[(2S,3R)-3-(1-methylcyclopropoxy)-2-[(2,2,2-trifluoroacetyl)amino]butanoyl]-3-azabicyclo[3.1.0]hexane-2-carboxylic acid

To a solution of (1R,2S,5S)-3-[(2S,3R)-2-amino-3-(1-methylcyclopropoxy)butanoyl]-6,6-dimethyl-3-azabicyclo[3.1.0]hexane-2-carboxylic acid (180 mg, 518.96 umol, 1 eq, HCl) in methyl 2,2,2-trifluoroacetate (664.53 mg, 5.19 mmol, 523.25 uL, 10 eq) was added TEA (472.62 mg, 4.67 mmol, 650.09 uL, 9 eq). The mixture was stirred at 20° C. for 6 h. Upon completion, the reaction mixture was concentrated in a vacuum to remove most of methyl 2,2,2-trifluoroacetate to afford a residue. The combined residue was diluted with ethyl acetate (10 mL) and washed with 1 M aqueous HCl (5 mL). The organic layer was separated and the aqueous layer was extracted with ethyl acetate (10 ml*2). The combined organic layer was dried over Na₂SO₄, filtered and concentrated under reduced pressure to give (2S)-2-[[(2S,3R)-3-tert-butoxy-2-[(2,2,2-trifluoroacetyl)amino]butanoyl]amino]-3-(1-fluorocyclopropyl)propanoic acid (230 mg, 442.35 umol, 61.20% yield, 77% purity) as a light yellow oil and used directly for next step. MS (ESI) m/z 407.2 [M+H]⁺.

Step 10: (1R,2S,5S)—N-[(1S)-1-cyano-2-[(3R)-5,5-dimethyl-2-oxo pyrrolidin-3-yl]ethyl]-6,6-dimethyl-3-[(2S,3R)-3-(1-methylcyclopropoxy)-2-[(2,2,2-trifluoroacetyl)amino]butanoyl]-3-azabicyclo[3.1.0]hexane-2-carboxamide (Compound 299) and (1R,2R,5S)—N-[(1S)-1-cyano-2-[(3R)-5,5-dimethyl-2-oxo pyrrolidin-3-yl]ethyl]-6,6-dimethyl-3-[(2S,3R)-3-(1-methylcyclopropoxy)-2-[(2,2,2-trifluoroacetyl)amino]butanoyl]-3-azabicyclo[3.1.0]hexane-2-carboxamide (Compound 299a)

To a solution of (1R,2S,5S)-6,6-dimethyl-3-[(2S,3R)-3-(1-methylcyclopropoxy)-2-[(2,2,2-trifluoroacetyl)amino]butanoyl]-3-azabicyclo[3.1.0]hexane-2-carboxylic acid (200 mg, 492.13 umol, 1 eq), (2S)-2-amino-3-[(3R)-5,5-dimethyl-2-oxo-pyrrolidin-3-yl]propanenitrile (89.19 mg, 492.13 umol, 1 eq) in acetonitrile (3 mL) was added 1-methylimidazole (121.22 mg, 1.48 mmol, 117.69 uL, 3 eq) and 1-[chloro(pyrrolidin-1-ium-1-ylidene)methyl]pyrrolidine; hexafluorophosphate (327.42 mg, 984.26 umol, 2 eq). The mixture was stirred at 20° C. for 1 h. Upon completion, the reaction mixture was added water (5 mL) and extected with ethyl acetate (5 mL*2). The organic phase was washed with brine (5 mL), dried over anhydrous magnesium sulfate, filtered, concentrated under reduced pressure. The residue was purified by prep-HPLC (column: Waters Xbridge BEH C18 100 30 mm*10 um; mobile phase: [water(NH₄HCO₃)-ACN]; B %: 20%-50%, 8 min) to give (1R,2S,5S)—N-[(1S)-1-cyano-2-[(3R)-5,5-dimethyl-2-oxo-pyrrolidin-3-yl]ethyl]-6,6-dimethyl-3-[(2S,3R)-3-(1-methylcyclopropoxy)-2-[(2,2,2-trifluoroacetyl)amino]butanoyl]-3-azabicyclo[3.1.0]hexane-2-carboxamide (Compound 299) (58.14 mg, 100.95 umol, 20.51% yield, 98.9% purity) as a white solid. MS (ESI) m/z 570.3 [M+H]⁺.

¹H NMR (400 MHz, DMSO-d₆) S=9.87 (br d, J=8.3 Hz, 1H), 9.00 (d, J=8.6 Hz, 1H), 7.84 (s, 1H), 4.95-4.89 (m, 1H), 4.34 (t, J=8.6 Hz, 1H), 4.10 (s, 1H), 3.93-3.86 (m, 2H), 3.70 (br d, J=10.4 Hz, 1H), 2.62-2.56 (m, 1H), 2.15-2.00 (m, 1H), 1.98 (br dd, J =8.6, 12.0 Hz, 1H), 1.77-1.67 (m, 1H), 1.56-1.52 (m, 2H), 1.33 (br d, J=7.5 Hz, 1H), 1.30 (s, 3H), 1.23-1.09 (m, 9H), 1.02-0.97 (m, 3H), 0.83 (s, 2H), 0.81-0.74 (m, 1H), 0.73-0.71 (m, 1H), 0.70 (br dd, J=4.9, 10.9 Hz, 1H), 0.66-0.58 (m, 1H), 0.39-0.26 (m, 2H).

(1R,2R,5S)—N-[(1S)-1-cyano-2-[(3R)-5,5-dimethyl-2-oxo-pyrrolidin-3-yl]ethyl]-6,6-dimethyl-3-[(2S,3R)-3-(1-methylcyclopropoxy)-2-[(2,2,2-trifluoroacetyl)amino]butanoyl]-3-azabicyclo[3.1.0]hexane-2-carboxamide (Compound 299a) (2.11 mg, 2.18 umol, 4.43e-1% yield, 58.8% purity) was obtained as a white solid. MS (ESI) m/z 570.3 [M+H]⁺.

¹H NMR (400 MHz, DMSO-d₆) S=9.75-9.66 (m, 1H), 8.72 (d, J=8.3 Hz, 1H), 7.80 (s, 1H), 4.94-4.88 (m, 1H), 4.43 (br d, J=9.4 Hz, 1H), 4.07-4.05 (m, 2H), 3.95-3.91 (m, 1H), 3.60-3.57 (m, 1H), 2.49-2.40 (m, 1H), 2.15-2.04 (m, 1H), 2.00 (dd, J=8.7, 12.3 Hz, 1H), 1.74 (ddd, J=6.6, 9.8, 13.5 Hz, 1H), 1.57-1.51 (m, 2H), 1.28-1.25 (m, 1H), 1.24 (s, 3H), 1.17 (s, 3H), 1.14-1.07 (m, 6H), 1.04-1.01 (m, 3H), 0.99 (br s, 3H), 0.64 (br s, 2H), 0.35-0.31 (m, 2H)

Example 49: Synthesis of (2S)—N-[(1S)-2-[[(1S)-1-cyan-2-[(6R)-5-oxo-4-azaspiro[2.4]heptan-6-yl]ethyl]amino]-1-[(1-fluorocyclopropyl)methyl]-2-oxo-ethyl]-3,3-dimethyl-2-[(2,2,2-trifluoroacetyl)amino]butanamide (Compound 300)

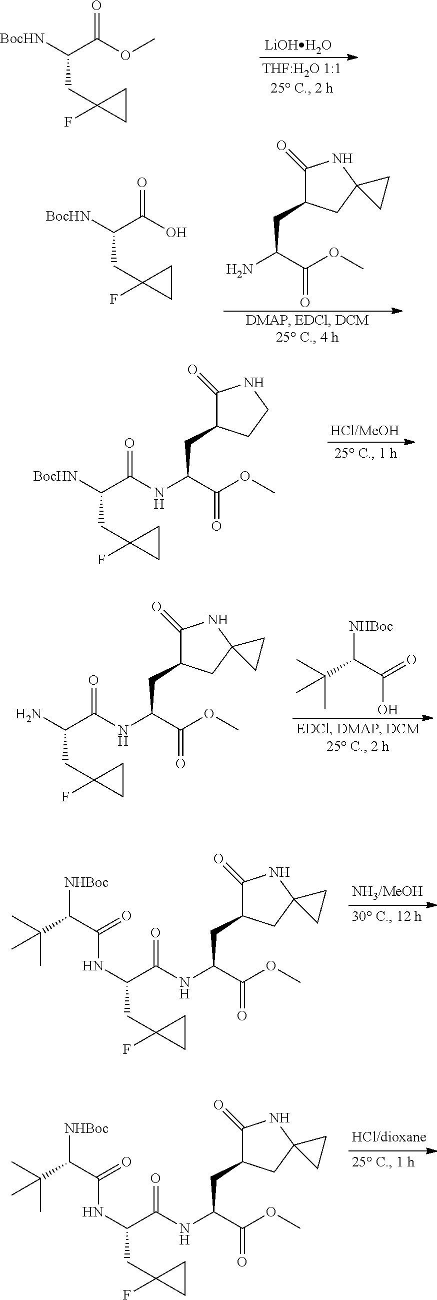

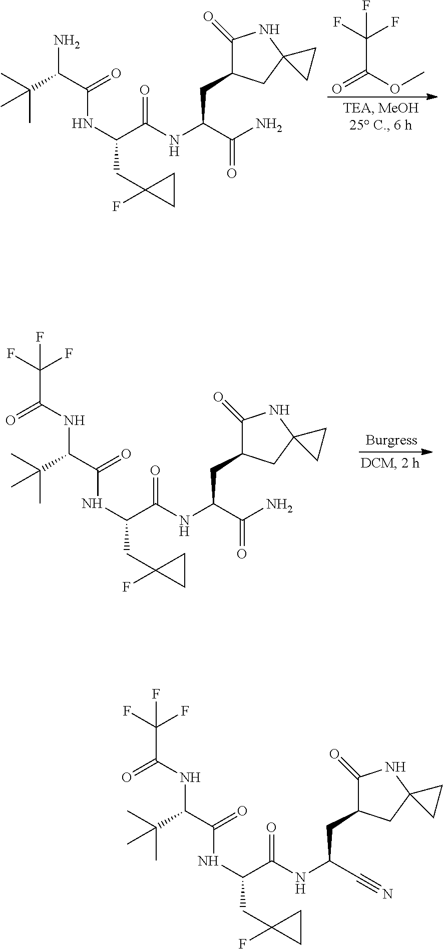

Step 1: (2S)-2-(tert-butoxycarbonylamino)-3-(1 fluorocyclopropyl)propanoic acid

To a solution of methyl (2S)-2-(tert-butoxycarbonylamino)-3-(1-fluorocyclopropyl) propanoate (630.47 mg, 2.41 mmol, 1 eq) and LiOH.H₂O (354.39 mg, 8.45 mmol, 3.5 eq) in THE (5 mL) was added with H₂O (5 mL), and the resulting mixture was stirred at 25° C. for 2 h. Upon completion, the residue was concentrated and adjusted to pH-4 with 2 N HCl, and was extracted with EA (20 mL*2), the combined organic phase was washed with brine (10 mL), dried over Na₂SO₄ and concentrated to give (2S)-2-(tert-butoxycarbonylamino)-3-(1-fluorocyclopropyl) propanoic acid (580 mg, crude) as a white solid.

Step 2: methyl 4,6,7-trifluoro-1 H-indole-2-carboxylate

To a solution of methyl (2S)-2-amino-3-[(6R)-5-oxo-4-azaspiro[2.4]heptan-6-yl]propanoate (497.86 mg, 2.35 mmol, 1 eq) and (2S)-2-(tert-butoxycarbonylamino)-3-(1-fluorocyclopropyl)propanoic acid (580 mg, 2.35 mmol, 1 eq) in DCM (8 mL) and then EDCI (899.34 mg, 4.69 mmol, 2 eq) and DMAP (859.72 mg, 7.04 mmol, 3 eq) was added, then the mixture was stirred at 25° C. for 2 h and additionally stirred for 2 h at 25° C. Upon completion, the reaction mixture was diluted with H₂O 10 mL and then extracted with EA (20 mL*2). The combined organic layers were washed with 15% citric acid (20 mL*2), NaHCO₃(20 mL) and brine (20 mL) dried over Na₂SO₄, filtered and concentrated under reduced pressure to give a residue. The residue was purified by silica gel column chromatography (SiO₂ PE:EA=0:1) to give methyl (2S)-2-[[(2S)-2-(tert-butoxycarbonylamino)-3-(1-fluorocyclopropyl)propanoyl]amino]-3-[(6R)-5-oxo-4-azaspiro[2.4]heptan-6-yl]propanoate (635 mg, 1.39 mmol, 59.36% yield, 96.81% purity) as a white solid.

Step 3: methyl (2S)-2-[[(2S)-2-amino-3-(1 fluorocyclopropyl)propanoyl]amino]-3-[(6R)-5-oxo-4-azaspiro[2.4]heptan-6-yl]propanoate

A solution of methyl (2S)-2-[[(2S)-2-(tert-butoxycarbonylamino)-3-(1-fluorocyclopropyl)propanoyl]amino]-3-[(6R)-5-oxo-4-azaspiro[2.4]heptan-6-yl]propanoate (635 mg, 1.44 mmol, 1 eq) in HCl/MeOH (4 M, 10 mL, 27.81 eq) was stirred at 25° C. for 1 h. Upon completion, the residue was concentrated in vacuum, and washed with DCM (10 mL*2) and concentrated to give methyl (2S)-2-[[(2S)-2-amino-3-(1-fluorocyclopropyl)propanoyl]amino]-3-[(6R)-5-oxo-4-azaspiro[2.4]heptan-6-yl]propanoate (490 mg, crude) as a white solid.

Step 4: methyl (2S)-2-[[(2S)-2-[[(2S)-2-(tert-butoxycarbonylamino)-3,3-dimethyl-butanoyl]amino]-3-(1 fluorocyclopropyl)propanoyl]amino]-3-[(6R)-5-oxo-4-azaspiro[2.4]heptan-6-yl]propanoate

To a solution of (2S)-2-(tert-butoxycarbonylamino)-3,3-dimethyl-butanoic acid (331.98 mg, 1.44 mmol, 1 eq) and methyl (2S)-2-[[(2S)-2-amino-3-(1-fluorocyclopropyl)propanoyl]amino]-3-[(6R)-5-oxo-4-azaspiro[2.4]heptan-6-yl]propanoate (490 mg, 1.44 mmol, 1 eq) in DCM (5 mL) was added with DMAP (526.08 mg, 4.31 mmol, 3 eq) and EDCI (550.32 mg, 2.87 mmol, 2 eq), and then the mixture was stirred at 25° C. for 2 h. Upon completion, the reaction mixture was diluted with H₂O 10 mL and then extracted with ethyl acetate (10 mL*2). The combined organic layers were washed with 15% citric acid (10 mL*2), NaHCO₃(10 mL) and brine (10 mL) dried over Na₂SO₄, filtered and concentrated under reduced pressure to give a residue. The residue was purified by silica gel column chromatography (SiO₂ PE:EA=0:1) to give methyl (2S)-2-[[(2S)-2-[[(2S)-2-(tert-butoxycarbonylamino)-3,3-dimethyl-butanoyl]amino]-3-(1-fluorocyclopropyl)propanoyl]amino]-3-[(6R)-5-oxo-4-azaspiro[2.4]heptan-6-yl]propanoate (691 mg, 1.14 mmol, 79.19% yield, 91.24% purity) as a white solid.

Step 5: tert-butyl N-((1S)-1-[[(1S)-2-[((1S)-2-amino-2-oxo-1-[[(6R)-5-oxo-4-azaspiro[2.4]heptan-6-yl]methyl]ethyl]amino]-1-[(1 fluorocyclopropyl)methyl]-2-oxo-ethyl]carbamoyl]-2,2-dimethyl propyl]carbamate

A solution of methyl (2S)-2-[[(2S)-2-[[(2S)-2-(tert-butoxycarbonylamino)-3,3-dimethyl-butanoyl]amino]-3-(1-fluorocyclopropyl)propanoyl]amino]-3-[(6R)-5-oxo-4-azaspiro[2.4]heptan-6-yl]propanoate (691 mg, 1.25 mmol, 1 eq) in NHilMeOH (5 mL) was stirred at 30° C. for 12 h. Upon completion, the residue was concentrated in vacuum, and washed with DCM (10 mL*2) and concentrated to give tert-butyl N-[(1S)-1-[[(1S)-2-[[(1S)-2-amino-2-oxo-1-[[(6R)-5-oxo-4-azaspiro[2.4]heptan-6-yl]methyl]ethyl]amino]-1-[(1-fluorocyclopropyl)methyl]-2-oxo-ethyl]carbamoyl]-2,2-dimethyl-propyl]carbamate (650 mg, crude) as a light yellow solid.

Step 6: (2S)-2-amino-N-[(1S)-2-[[(1S)-2-amino-2-oxo-1-[[(6R)-5-oxo-4-azaspiro[2.4]heptan-6-yl]methyl]ethyl]amino]-1-[(1 fluorocyclopropyl)methyl]-2-oxo-ethyl]-3,3-dimethyl-butanamide

A solution of tert-butyl N-[(1S)-1-[[(1S)-2-[[(1S)-2-amino-2-oxo-1-[[(6R)-5-oxo-4-aza5piro[2.4]heptan-6-yl]methyl]ethyl]amino]-1-[(1-fluorocyclopropyl)methyl]-2-oxo-ethyl]carbamoyl]-2,2-dimethyl-propyl]carbamate (650 mg, 1.20 mmol, 1 eq) in HCl/dioxane (4 M, 10 mL, 33.21 eq) was stirred at 25° C. for 1 h. Upon completion, the residue was concentrated and washed with DCM (10 mL*2) and concentrated to give (2S)-2-amino-N [(1S)-2-[[(1S)-2-amino-2-oxo-1-[[(6R)-5-oxo-4-azaspiro[2.4]heptan-6-yl]methyl]ethyl]amino]-1-[(1-fluorocyclopropyl)methyl]-2-oxo-ethyl]-3,3-dimethyl-butanamide (520 mg, crude) as a white solid.

Step 7: (2S)-2-amino-N-[(1S)-2-[((1S)-2-amino-2-oxo-1-[[(6R)-5-oxo-4-azaspiro[2.4]heptan-6-yl]methyl]ethyl]amino]-1-[(1 fluorocyclopropyl)methyl]-2-oxo-ethyl]-3,3-dimethyl-butanamide

To a solution of (2S)-2-amino-N-[(1S)-2-[[(1S)-2-amino-2-oxo-1-[[(6R)-5-oxo-4-azaspiro[2.4]heptan-6-yl]methyl]ethyl]amino]-1-[(1-fluorocyclopropyl)methyl]-2-oxo-ethyl]-3,3-dimethyl-butanamide (520 mg, 1.18 mmol, 1 eq) in MeOH (1 mL) was added TEA (359.15 mg, 3.55 mmol, 494.02 uL, 3 eq), and then methyl 2,2,2-trifluoroacetate (454.49 mg, 3.55 mmol, 357.86 uL, 3 eq) was added. The resulting mixture was stirred at 25° C. for 4 h and then for additional 2 h at the same temperature. Upon completion, the residue was concentrated in vacuum, and washed with DCM (10 mL*2) and concentrated to give (2S)—N-[(1S)-2-[[(1S)-2-amino-2-oxo-1-[[(6R)-5-oxo-4-azaspiro[2.4]heptan-6-yl]methyl]ethyl]amino]-1-[(1-fluorocyclopropyl)methyl]-2-oxo-ethyl]-3,3-dimethyl-2-[(2,2,2-trifluoroacetyl)amino]butanamide (620 mg, crude) as a white solid.

Step 8: (2S)—N-[(1S)-2-[[(1S)-1-cyano-2-[(6R)-5-oxo-4-azaspiro[2.4]heptan-6-yl]ethyl]amino]-1-[(1 fluorocyclopropyl)methyl]-2-oxo-ethyl]-3,3-dimethyl-2-[(2,2,2-trifluoroacetyl)amino]butanamide

To a solution of (2S)—N-[(1S)-2-[[(1S)-2-amino-2-oxo-1-[[(6R)-5-oxo-4-azaspiro[2.4]heptan-6-yl]methyl]ethyl]amino]-1-[(1-fluorocyclopropyl)methyl]-2-oxo-ethyl]-3,3-dimethyl-2-[(2,2,2-trifluoroacetyl)amino]butanamide (620 mg, 1.16 mmol, 1 eq) in DCM (5 mL) was added Burgess reagent (827.70 mg, 3.47 mmol, 3 eq), and then the mixture was stirred at 25° C. for 2 h. Upon completion, the reaction was quenched with water (0.8 mL), and was blowed to dry with N₂ the residue was purified by prep-HPLC column: Waters Xbridge Prep OBD C18 150*40 mm*10 um; mobile phase: [water(NH₄HCO₃)-ACN]; B %: 20%-50%, 8 min to give (2S)—N-[(1S)-2-[[(1S)-1-cyano-2-[(6R)-5-oxo-4-azaspiro[2.4]heptan-6-yl]ethyl]amino]-1-[(1-fluorocyclopropyl)methyl]-2-oxo-ethyl]-3,3-dimethyl-2-[(2,2,2-trifluoroacetyl)amino]butanamide (130.30 mg, 251.78 umol, 21.75% yield, 100% purity) as a white solid MS (ESI) m/z 518.2 [M+H]⁺.

¹H NMR (400 MHz, DMSO-d₆) δ=9.23 (brd, J=9.2 Hz, 1H), 9.10 (d, J=8.3 Hz, 1H), 8.47 (d, J=7.0 Hz, 1H), 7.76 (s, 1H), 4.96-4.87 (m, 1H), 4.45-4.38 (m, 2H), 2.33 (br s, 1H), 2.28-2.18 (m, 1H), 2.14-2.02 (m, 2H), 2.02-1.89 (m, 2H), 1.80 (ddd, J=5.4, 10.7, 13.3 Hz, 1H), 0.93 (s, 10H), 0.89-0.85 (m, 1H), 0.77-0.70 (m, 2H), 0.61-0.49 (m, 4H)

Example 51: Synthesis of (2S,3R)-3-tert-butoxy-N-[(1S)-2-[[(1S)-1-cyan-2-[(3R)-5,5-dimethyl-2-oxo-pyrrolidin-3-yl]ethyl]amino]-1-[(1-fluorocyclopropyl)methyl]-2-oxo-ethyl]-2-[(2,2,2-trifluoroacetyl)amino]butanamide (Compound 301) and (2S,3R)-3-tert-butoxy-N-[(1R)-2-[[(1S)-1-cyano-2-[(3R)-5,5-dimethyl-2-oxo-pyrrolidin-3-yl]ethyl]amino]-1-[(1-fluorocyclopropyl)methyl]-2-oxo-ethyl]-2-[(2,2,2-trifluoroacetyl)amino]butanamide (Compound 301a)

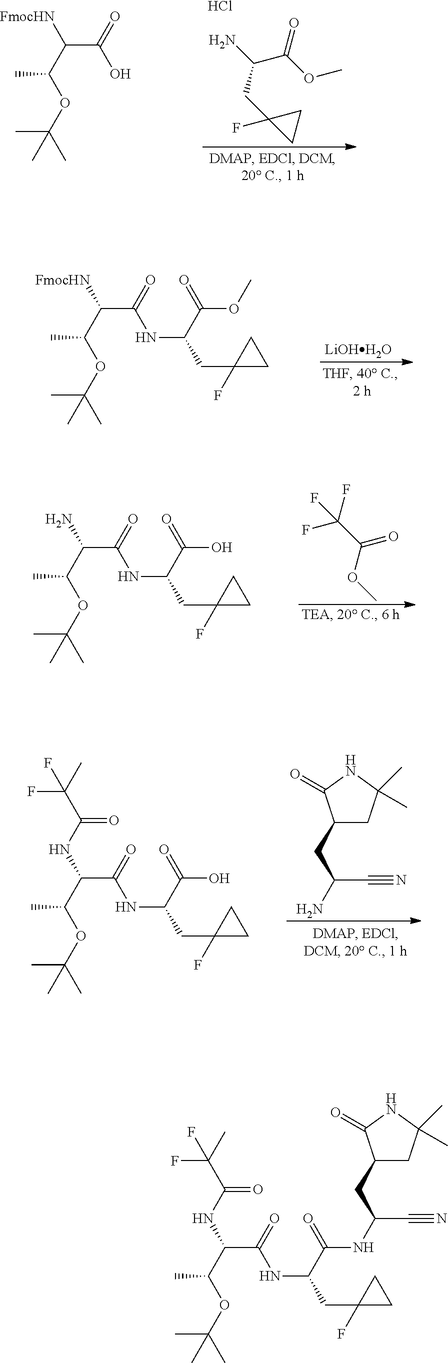

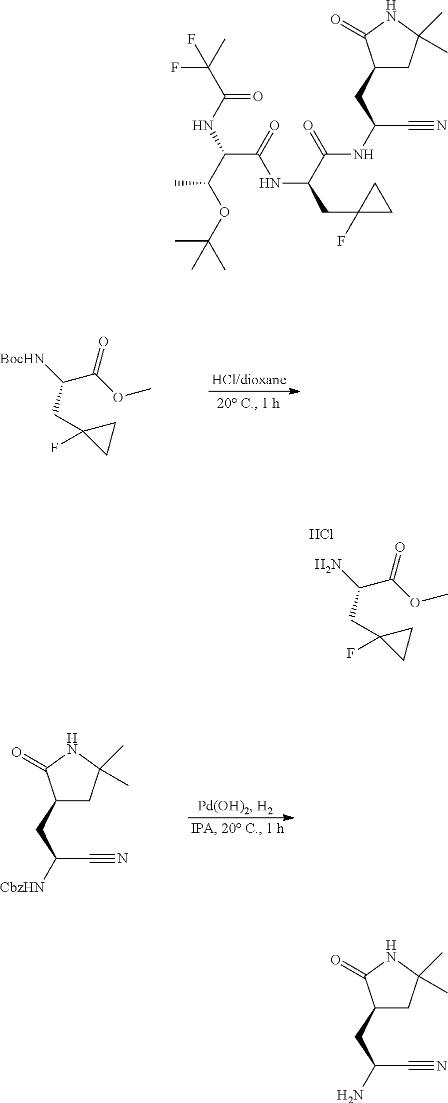 [SMILES: C[C@@H](OC(C)(C)C)[C@H](NC(=O)C(C)(F)F)C(=O)N[C@H](CC1(F)CC1)C(=O)N[C@H](C#N)C[C@@H]1CC(C)(C)NC1=O ; COC(=O)[C@H](CC1(F)CC1)NC(=O)OC(C)(C)C ; Cl ; COC(=O)[C@@H](N)CC1(F)CC1 ; CC1(C)C[C@@H](C[C@@H](C#N)NC(=O)OCc2ccccc2)C(=O)N1 ; CC1(C)C[C@@H](C[C@H](N)C#N)C(=O)N1]

Step 1: methyl (2S)-2-amino-3-(1 fluorocyclopropyl)propanoate

A solution of methyl (2S)-2-(tert-butoxycarbonylamino)-3-(1-fluorocyclopropyl)propanoate (500 mg, 1.91 mmol, 1 eq) in HCl/dioxane (4 M, 5.00 mL, 10.45 eq) was stirred at 20° C. for 1 h. Upon completion, the reaction mixture was concentrated under reduced pressure to give methyl (2S)-2-amino-3-(1-fluorocyclopropyl) propanoate (380 mg, crude, HCl) as a white solid. MS (ESI) m/z 162.1 [M+H]⁺

Step 2: (2S)-2-amino-3-[(3R)-5,5-dimethyl-2-oxo pyrrolidin-3-yl]propanenitrile

To a solution of benzyl N-[(1S)-1-cyano-2-[(3R)-5,5-dimethyl-2-oxo-pyrrolidin-3-yl]ethyl]carbamate (300 mg, 951.28 umol, 1 eq) in IPA (5 mL) was added Pd(OH)₂ (420.83 mg, 599.30 umol, 20% purity, 0.63 eq) under N₂. The suspension was degassed under vacuum and purged with H₂ several times. The mixture was stirred under H₂ (15 psi) at 20° C. for 1 h. Upon completion, the reaction mixture was filtered and concentrated under reduced pressure to give (2S)-2-amino-3-[(3R)-5,5-dimethyl-2-oxo-pyrrolidin-3-yl]propanenitrile (200 mg, crude) as a colourless oil. MS (ESI) m/z 182.1 [M+H]⁺

Step 3: methyl (2S)-2-[[(2S,3R)-3-tert-butoxy-2-(9H fluoren-9-ylmethoxycarbonylamino)butanoyl]amino]-3-(1 fluorocyclopropyl)propanoate

To a solution of (2S,3R)-3-tert-butoxy-2-(9H-fluoren-9-ylmethoxycarbonylamino)butanoic acid (0.5 g, 1.26 mmol, 1 eq) and methyl (2S)-2-amino-3-(1-fluorocyclopropyl)propanoate (248.62 mg, 1.26 mmol, 1 eq, HCl) in DCM (10 mL) was added DMAP (461.05 mg, 3.77 mmol, 3 eq) and EDCI (482.31 mg, 2.52 mmol, 2 eq). The mixture was stirred at 20° C. for 1 h. Upon completion, the reaction mixture was diluted with H₂O (10 mL) and extracted with DCM (10 mL*3). The combined organic layers were washed with brine (10 mL), dried over Na₂SO₄, filtered and concentrated under reduced pressure to give a residue. The residue was purified by column chromatography (SiO₂, Petroleum ether/Ethyl acetate=3011 to 1011) to give methyl (2S)-2-[[(2S,3R)-3-tert-butoxy-2-(9H-fluoren-9-ylmethoxycarbonylamino)butanoyl]amino]-3-(1-fluorocyclopropyl)propanoate (400 mg, 688.10 umol, 54.70% yield, 93% purity) as a yellow oil. MS (ESI) m/z 541.3 [M+H]⁺.

Step 4: (2S)-2-[[(2S,3R)-2-amino-3-tert-butoxy-butanoyl]amino]-3-(1-fluorocyclopropyl)propanoic acid

To a solution of methyl (2S)-2-[[(2S,3R)-3-tert-butoxy-2-(9H-fluoren-9-ylmethoxycarbonylamino)butanoyl]amino]-3-(1-fluorocyclopropyl)propanoate (0.4 g, 688.10 umol, 93% purity, 1 eq) in THE (20 mL) and H₂O (6 mL) was added LiOH.H₂O (101.06 mg, 2.41 mmol, 3.5 eq). The mixture was stirred at 40° C. for 2 h. Upon completion, the reaction solution was added 1 N HCl to pH-4 and extracted with ethyl acetate (5 mL*2). The organic phase was washed with salt water (5 ml), dried on anhydrous sodium sulfate, filtered, and concentrated under pressure to give (2S)-2-[[(2S,3R)-2-amino-3-tert-butoxy-butanoyl]amino]-3-(1-fluorocyclopropyl)propanoic acid (220 mg, crude) as a white solid. MS (ESI) m/z 305.2 [M+H]⁺.

Step 5: (2S)-2-[[(2S,3R)-3-tert-butoxy-2-[(2,2,2-trifluoroacetyl)amino]butanoyl]amino]-3-(1 fluorocyclopropyl)propanoic acid

To a mixture of (2S)-2-[[(2S,3R)-2-amino-3-tert-butoxy-butanoyl]amino]-3-(1-fluorocyclopropyl)propanoic acid (220 mg, 722.84 umol, 1 eq) in methyl 2,2,2-trifluoroacetate (925.59 mg, 7.23 mmol, 728.81 uL, 10 eq) was added TEA (658.29 mg, 6.51 mmol, 905.48 uL, 9 eq). The resulting mixture was stirred at 20° C. for 6 h. Upon completion, the reaction mixture was concentrated in a vacuum to remove most of methyl 2,2,2-trifluoroacetate to afford a residue. The combined residue was diluted with ethyl acetate (10 mL) and washed with 1 M aqueous HCl (5 mL). The organic layer was seprated and the aqueous layer was extracted with ethyl acetate (10 ml*2). The combined orangic layers were dried over Na₂SO₄, filtered and concentrated under reduced pressure to give a residue. The residue was purified by column chromatography (SiO₂, Dichloromethane: Methanol=I/O to 5/1) to give (2S)-2-[[(2S,3R)-3-tert-butoxy-2-[(2,2,2-trifluoroacetyl)amino]butanoyl]amino]-3-(1-fluorocyclopropyl)propanoic acid (230 mg, 442.35 umol, 61.20% yield, 77% purity) as a light yellow oil. MS (ESI) m/z 401.2 [M+H]⁺.

Step 6: (2S,3R)-3-tert-butoxy-N-[(1S)-2-[[(1S)-1-cyano-2-[(3R)-5,5-dimethyl-2-oxo-pyrrolidin-3-yl]ethyl]amino]-1-[(1 fluorocyclopropyl)methyl]-2-oxo-ethyl]-2-[(2,2,2-trifluoroacetyl)amino]butanamide (Compound 301) and 2S,3R)-3-tert-butoxy-N-[(1R)-2-[[(1S)-1-cyano-2-[(3R)-5,5-dimethyl-2-oxo pyrrolidin-3-yl]ethyl]amino]-1-[(1-fluorocyclopropyl)methyl]-2-oxo-ethyl]-2-[(2,2,2-trifluoroacetyl)amino]butanamide (Compound 301a)

To a solution of (2S)-2-[[(2S,3R)-3-tert-butoxy-2-[(2,2,2-trifluoroacetyl)amino]butanoyl]amino]-3-(1-fluorocyclopropyl)propanoic acid (190 mg, 365.42 umol, 77% purity, 1 eq) and (2S)-2-amino-3-[(3R)-5,5-dimethyl-2-oxo-pyrrolidin-3-yl]propanenitrile (66.23 mg, 365.42 umol, 1 eq) in DCM (10 mL) was added DMAP (133.93 mg, 1.10 mmol, 3 eq) and EDCI (140.10 mg, 730.83 umol, 2 eq). The mixture was stirred at 20° C. for 1 h. Upon completion, the reaction mixture was diluted with H₂O (10 mL) and extracted with DCM (10 mL*3). The combined organic layers were washed with brine (10 mL), dried over Na₂SO₄, filtered and concentrated under reduced pressure to give a residue. The residue was purified by prep-HPLC (column: Phenomenex C18 75*30 mm*3 um; mobile phase: [water(NH₄HCO₃)-ACN]; B %: 35%-55%, 8 min) to give (2S,3R)-3-tert-butoxy-N-[(1S)-2-[[(1S)-1-cyano-2-[(3R)-5,5-dimethyl-2-oxo-pyrrolidin-3-yl]ethyl]amino]-1-[(1-fluorocyclopropyl)methyl]-2-oxo-ethyl]-2-[(2,2,2-trifluoroacetyl)amino]butanamide (Compound 301) (58.8 mg, 104.33 umol, 28.55% yield, 100% purity) as a white solid. MS (ESI) m/z 564.2 [M+H]⁺.

¹H NMR (400 MHz, DMSO-d₆) S=9.20-9.11 (m, 1H), 9.09-9.01 (m, 1H), 8.48-8.40 (m, 1H), 7.87-7.77 (m, 1H), 4.96 (s, 1H), 4.52-4.42 (m, 1H), 4.40-4.31 (m, 1H),3.90-3.80 (m, 1H), 2.56-2.54 (m, 1H), 2.13-2.10 (m, 2H), 2.07 (brd, J=6.8 Hz, 1H), 2.06-1.99 (m, 1H), 1.79-1.68 (m, 1H), 1.57-1.48 (m, 1H), 1.18-1.12 (m, 6H), 1.09 (s, 9H), 1.07 (br d, J=6.0 Hz, 3H), 0.94 (br d, J=18.8 Hz, 2H), 0.72-0.70 (m, 1H), 0.60 0.55 (m, 1H)

(2S,3R)-3-tert-butoxy-N-[(1R)-2-[[(1S)-1-cyano-2-[(3R)-5,5-dimethyl-2-oxo-pyrrolidin-3-yl]ethyl]amino]-1-[(1-fluorocyclopropyl)methyl]-2-oxo-ethyl]-2-[(2,2,2-trifluoroacetyl)amino]butanamide (Compound 301a) (32.14 mg, 56.29 umol, 15.40% yield, 98.7% purity) was obtained as a white solid. MS (ESI) m/z 564.2 [M+H]⁺.

¹H NMR (400 MHz, DMSO-d₆) S=9.18-8.95 (m, 2H), 8.47-8.38 (m, 1H), 7.84 (s, 1H), 4.96 (q, J=7.8 Hz, 1H), 4.59-4.54 (m, 1H), 4.40 (br d, J=5.1 Hz, 1H), 3.94 (quin, J=5.9 Hz, 1H), 2.66-2.50 (m, 1H), 2.28-2.25 (m, 1H), 2.10-2.05 (m, 1H), 2.03 (br dd, J=8.7, 12.3 Hz, 1H), 1.99-1.86 (m, 1H), 1.79 (td, J=7.9, 13.8 Hz, 1H), 1.52 (br t, J=11.4 Hz, 1H), 1.18 (s, 3H), 1.12 (s, 9H), 1.11 (br s, 3H), 1.05 (d, J=6.1 Hz, 3H), 0.91 (br d, J=19.2 Hz, 2H), 0.75 (br t, J=9.7 Hz, 1H), 0.60-0.57 (m, 1H)

Example 50: Synthesis of (1R,2S,5S)—N-[(1S)-1-cyano-2-[(3R)-5,5-dimethyl-2-oxo-pyrrolidin-3-yl]ethyl]-3-[(2S,3R)-3-(cyclobutoxy)-2-[(2,2,2-trifluoroacetyl)amino]butanoyl]-6,6-dimethyl-3-azabicyclo[3.1.0]hexane-2-carboxamide (Compound 307)

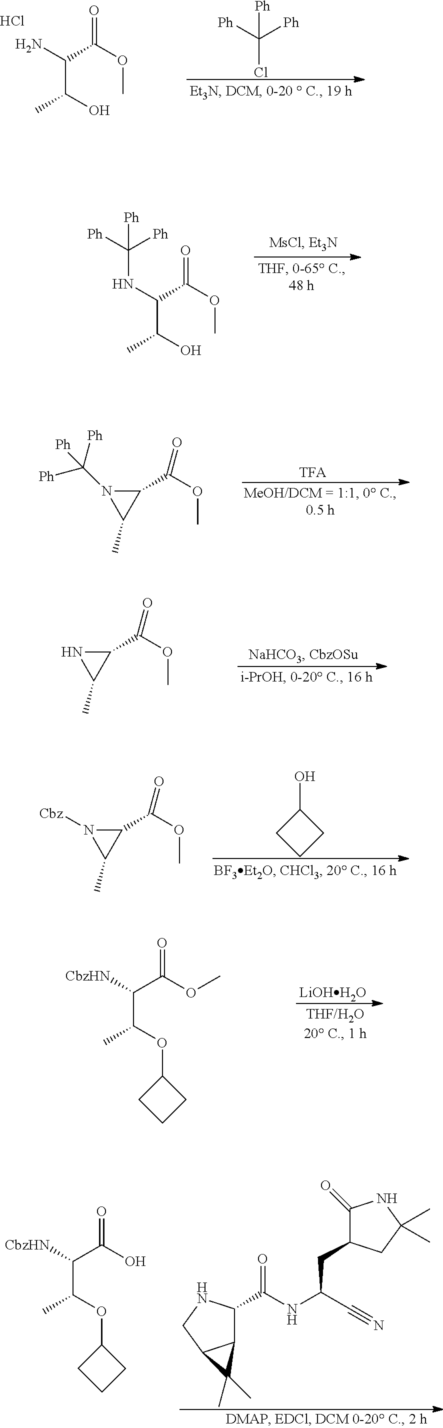

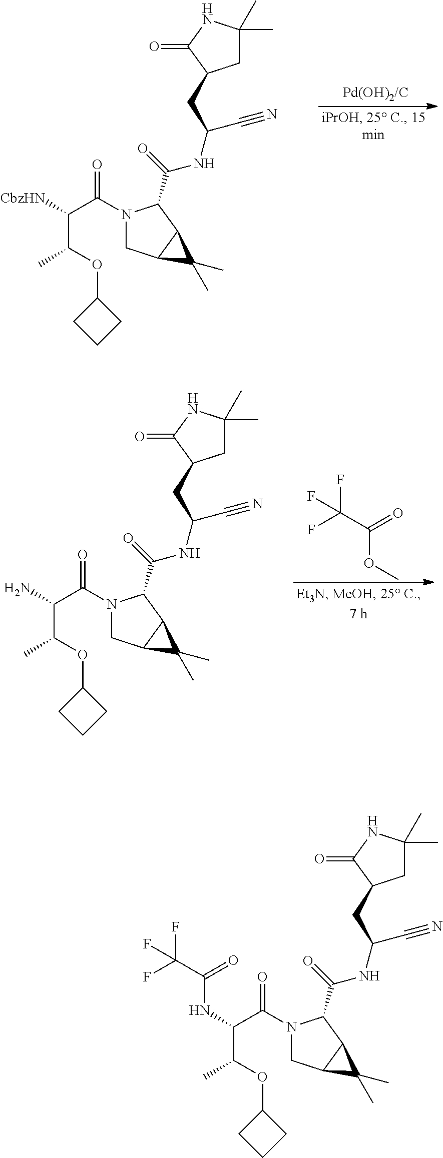

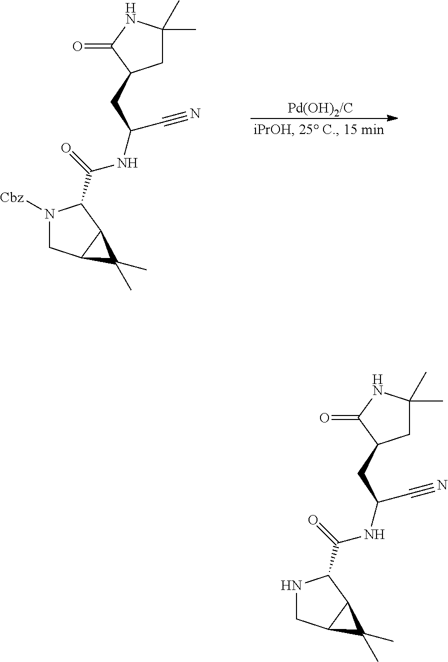 [SMILES: CC1(C)C[C@@H](C[C@@H](C#N)NC(=O)[C@@H]2[C@@H]3[C@H](CN2C(=O)OCc2ccccc2)C3(C)C)C(=O)N1 ; CC1(C)C[C@@H](C[C@@H](C#N)NC(=O)[C@H]2NC[C@H]3[C@@H]2C3(C)C)C(=O)N1]

Step 1: methyl (2S,3R)-3-hydroxy-2-(tritylamino)butanoate

To a solution of methyl (2S, 3R)-2-amino-3-hydroxy-butanoate (20 g, 117.92 mmol, 1 eq, HCl) in DCM (500 mL) was added dropwise Et₃N (23.86 g, 235.84 mmol, 32.83 mL, 2 eq) at 20° C. over 5 min under an atmosphere of nitrogen. After addition, the mixture was cooled to 0° C. and then [chloro(diphenyl)methyl]benzene (32.87 g, 117.92 mmol, 1 eq) was added over 5 min. The resulting mixture was stirred at 20° C. for 18 h 50 min. Upon completion, the reaction mixture was quenched by addition H₂O (200 mL) at 0° C. The combined organic layers were washed with 10% aqueous citric acid solution (2*200 ml) and brine (2*200 ml), and then extracted with DCM (300 mL), dried over Na₂SO₄, filtered and concentrated under reduced pressure to give a residue. The residue was purified by column chromatography (SiO₂, Petroleum ether/Ethyl acetate=I/O to 5/1) to give methyl (2S,3R)-3-hydroxy-2-(tritylamino)butanoate (41 g, 101.56 mmol, 86.12% yield, 93% purity) as a colorless oil.

Step 2: methyl (2S,3S)-3-methyl-1-trityl-aziridine-2-carboxylate

To a solution of methyl (2S,3R)-3-hydroxy-2-(tritylamino)butanoate (30 g, 79.90 mmol, 1 eq) in THE (250 mL) was added dropwise Et₃N (16.17 g, 159.80 mmol, 22.24 mL, 2 eq) at 0° C. under an atmosphere of nitrogen. After the addition, MsCl (9.15 g, 79.90 mmol, 6.18 mL, 1 eq) was added. The resulting mixture was stirred at 65° C. for 48 h. Upon completion, the solvent was removed in vacuo to leave a residue which was taken up in ethyl acetate (400 mL) and washed with 10% aqueous citric acid solution (3*200 mL) followed by saturated aqueous sodium bicarbonate solution (2*200 mL), dried over Na₂SO₄, filtered and concentrated under reduced pressure to give a residue. The residue was purified by column chromatography (SiO₂, Petroleum ether/Ethyl acetate=I/O to 5/1) to give methyl (2S,3S)-3-methyl-1-trityl-aziridine-2-carboxylate (22.2 g, 42.23 mmol, 52.86% yield, 68% purity) as a white gum.

Step 3: methyl (2S,3S)-3-methylaziridine-2-carboxylate

A solution of methyl (2S,3S)-3-methyl-1-trityl-aziridine-2-carboxylate (10 g, 27.98 mmol, 1 eq) in DCM (50 mL) and MeOH (50 mL) was cooled to 0° C., and then TFA (77.00 g, 675.30 mmol, 50.00 mL, 24.14 eq) was added drop-wise at 0° C. The mixture was stirred at 0° C. for 0.5 h. Upon completion, the reaction mixture was concentrated under reduced pressure to give methyl (2S,3S)-3-methylaziridine-2-carboxylate (3 g, crude) as a yellow solid. MS (ESI) m/z 116.1 [M+H]⁺.

Step 4: O1-benzyl O2-methyl (2S,3S)-3-methylaziridine-1,2-dicarboxylate

To a solution of methyl (25,35)-3-methylaziridine-2-carboxylate (3 g, 26.06 mmol, 1 eq) in i-PrOH (100 mL) and H₂O (100 mL) was added NaHCO₃(10.95 g, 130.29 mmol, 5.07 mL, 5 eq) to adjust pH=8 at 0° C., and then CbzOSu (7.14 g, 28.66 mmol, 1.1 eq) was added at 0° C., the mixture was stirred at 20° C. for 16 h. The reaction mixture was quenched by addition H₂O (100 mL), and then extracted with EA (150 mL*3). The combined organic layers were washed with brine (200 mL), dried over Na₂SO₄ filtered and concentrated under reduced pressure to give a residue. Upon completion, the residue was purified by column chromatography (SiO₂, Petroleum ether/Ethyl acetate=I/O to 10/1) to give O1-benzyl O2-methyl (2S,3S)-3-methylaziridine-1,2-dicarboxylate (5.4 g, 21.66 mmol, 83.14% yield) as a white oil. MS (ESI) m/z 250.1 [M+H]⁺.

Step 5: methyl (2S,3R)-2-(benzyloxycarbonylamino)-3-(cyclobutoxy)butanoate

To a solution of O1-benzyl O2-methyl (2S,3S)-3-methylaziridine-1,2-dicarboxylate (500 mg, 2.01 mmol, 1 eq) and cyclobutanol (159.10 mg, 2.21 mmol, 1.1 eq) in CHCl₃ (12 mL) was added BF₃.Et₂O (60.57 mg, 200.59 umol, 52.67 uL, 47% purity, 0.1 eq), and then the mixture was stirred at 20° C. for 16 h. Upon completion, the reaction mixture was quenched by addition H₂O (20 mL), and then extracted with DCM (20 mL*3). The combined organic layers were washed with NaHCO₃(25 mL*2) and brine (50 mL), dried over Na₂SO₄, filtered and concentrated under reduced pressure to give a residue. The residue was purified by prep-TLC (SiO₂, Petroleum ether/Ethyl acetate=3/1) to give methyl (2S,3R)-2-(benzyloxycarbonylamino)-3-(cyclobutoxy)butanoate (200 mg, 622.34 umol, 31.03% yield) was obtained as a colourless oil. MS (ESI) m/z 322.1 [M+H]⁺.

Step 6: (2S,3R)-2-(benzyloxycarbonylamino)-3-(cyclobutoxy)butanoic acid

To a solution of methyl (2S,3R)-2-(benzyloxycarbonylamino)-3-(cyclobutoxy)butanoate (200 mg, 622.34 umol, 1 eq) in THE (3 mL) and H₂O (1 mL) was added LiOH.H₂O (78.35 mg, 1.87 mmol, 3 eq). The mixture was stirred at 20° C. for 1 h. Upon completion, the reaction mixture was diluted with H₂O (15 mL) and adjusted pH=3, and then extracted with EA (20 mL*3). The combined organic layers were washed with brine (30 mL), dried over Na₂SO₄, filtered and concentrated under reduced pressure to give (2S,3R)-2-(benzyloxycarbonylamino)-3-(cyclobutoxy)butanoic acid (160 mg, crude) as a colorless oil. MS (ESI) m/z 308.1 [M+H]⁺.

Step 7: (1R,2S,5S)—N-[(1S)-1-cyano-2-[(3R)-5,5-dimethyl-2-oxo pyrrolidin-3-yl]ethyl]-6,6-dimethyl-3-azabicyclo[3.1.0]hexane-2-carboxamide

To a solution of benzyl (1R,2S,5S)-2-[[(1S)-1-cyano-2-[(3R)-5,5-dimethyl-2-oxo-pyrrolidin-3-yl]ethyl]carbamoyl]-6,6-dimethyl-3-azabicyclo[3.1.0]hexane-3-carboxylate (250 mg, 552.43 umol, 1 eq) in i-PrOH (3 mL) was added Pd(OH)₂ (75.00 mg, 20% purity) under N₂ atmosphere. The suspension was degassed and purged with H₂ for 3 times. The mixture was stirred under H₂ (15 Psi) at 25° C. for 15 min. Upon completion, the reaction was filtered and concentrated under reduced pressure to give (1R,2S,5S)—N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (180 mg, crude) as a white solid. MS (ESI) m/z 319.3 [M+H]⁺.

Step 8: benzyl N-[(1S,2R)-1-[(1R,2S,5S)-2-[[(1S)-1-cyan-2-[(3R)-5,5-dimethyl-2-oxo-pyrrolidin-3-yl]ethyl]carbamoyl]-6,6-dimethyl-3-azabicyclo[3.1.0]hexane-3-carbonyl]-2-(cyclobutoxy)propyl]carbamate

To a solution of (2S,3R)-2-(benzyloxycarbonylamino)-3-(cyclobutoxy)butanoic acid (160 mg, 520.59 umol, 1 eq) in DCM (6 mL) was added (1R,2S,5S)—N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (165.76 mg, 520.59 umol, 1 eq), and then EDCI (199.60 mg, 1.04 mmol, 2 eq) and DMAP (190.80 mg, 1.56 mmol, 3 eq) was added at 0° C. The mixture was stirred at 20° C. for 2 h. Upon completion, the reaction mixture was quenched by addition H₂O (10 mL), and extracted with DCM (15 mL*2). The combined organic layers were washed with brine (20 mL*2), dried over Na₂SO₄, filtered and concentrated under reduced pressure to give a residue. The residue was purified by column chromatography (SiO₂, Petroleum ether/Ethyl acetate=20/1 to 0/1) to give benzyl N-[(1S,2R)-1-[(1R,2S,5S)-2-[[(1S)-1-cyano-2-[(3R)-5,5-dimethyl-2-oxo-pyrrolidin-3-yl]ethyl]carbamoyl]-6,6-dimethyl-3-azabicyclo[3.1.0]hexane-3-carbonyl]-2-(cyclobutoxy)propyl]carbamate (90 mg, 148.09 umol, 28.45% yield) as a white solid. MS (ESI) m/z 608.3 [M+H]⁺.

Step 9: (1R,2S,5S)-3-[(2S,3R)-2-amino-3-(cyclobutoxy)butanoyl]-N-[(1S)-1-cyan-2-[(3R)-5,5-dimethyl-2-oxo pyrrolidin-3-yl]ethyl]-6,6-dimethyl-3-azabicyclo[3.1.0]hexane-2-carboxamide

To a solution of benzyl N-[(1S,2R)-1-[(1R,2S,5S)-2-[[(1S)-1-cyano-2-[(3R)-5,5-dimethyl-2-oxo-pyrrolidin-3-yl]ethyl]carbamoyl]-6,6-dimethyl-3-azabicyclo[3.1.0]hexane-3-carbonyl]-2-(cyclobutoxy)propyl]carbamate (90 mg, 148.09 umol, 1 eq) in i-PrOH (2 mL) was added Pd(OH)₂ (38.57 mg, 20% purity) under N₂ atmosphere. The suspension was degassed and purged with H₂ for 3 times. The mixture was stirred under H₂ (15 Psi) at 25° C. for 15 min. Upon completion, the reaction was filtered and concentrated under reduced pressure to give (1R,2S,5S)-3-[(2S,3R)-2-amino-3-(cyclobutoxy)butanoyl]-N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (50 mg, crude) as a white solid. MS (ESI) m/z 474.4 [M+H]⁺.

Step 10: (1R,2S,5S)—N-[(1S)-1-cyano-2-[(3R)-5,5-dimethyl-2-oxo pyrrolidin-3-yl]ethyl]-3-[(2S,3R)-3-(cyclobutoxy)-2-[(2,2,2-trifluoroacetyl)amino]butanoyl]-6,6-dimethyl-3-azabicyclo[3.1.0]hexane-2-carboxamide

To a solution of (1R,2S,5S)-3-[(2S,3R)-2-amino-3-(cyclobutoxy)butanoyl]-N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (75 mg, 158.36 umol, 1 eq) in MeOH (2.5 mL) was added drop-wise Et₃N (48.07 mg, 475.08 umol, 66.12 uL, 3 eq), and then methyl 2,2,2-trifluoroacetate (202.78 mg, 1.58 mmol, 159.67 uL, 10 eq) was added drop-wise. The resulting mixture was stirred at 25° C. for 7 h. Upon completion, the reaction was concentrated under reduced pressure to give a residue. The residue was purified by prep-HPLC (column: Waters Xbridge BEH C18 100*30 mm*10 um; mobile phase: [water(NH₄HCO₃)-ACN]; B %: 40%-70%, 8 min) to give (1R,2S,5S)—N-[(1S)-1-cyano-2-[(3R)-5,5-dimethyl-2-oxo-pyrrolidin-3-yl]ethyl]-3-[(2S,3R)-3-(cyclobutoxy)-2-[(2,2,2-trifluoroacetyl)amino]butanoyl]-6,6-dimethyl-3-azabicyclo[3.1.0]hexane-2-carboxamide (41 mg, 71.98 umol, 45.45% yield, 100% purity) as a white solid. MS (ESI) m/z 570.2 [M+H]⁺.

¹H NMR (400 MHz, DMSO-d₆) δ=9.86 (br s, 1H), 9.00 (d, J=8.5 Hz, 1H), 7.80 (s, 1H), 4.97-4.87 (m, 1H), 4.36 (br d, J=3.9 Hz, 1H), 4.11 (s, 1H), 3.98 (t, J=7.3 Hz, 1H), 3.92 (dd, J=5.5, 10.5 Hz, 1H), 3.78 (d, J=10.5 Hz, 1H), 3.68 (br dd, J=6.3, 8.9 Hz, 1H), 2.56-2.54 (m, 1H), 2.16-1.94 (m, 4H), 1.84-1.68 (m, 3H), 1.61-1.49 (m, 3H), 1.42 1.30 (m, 2H), 1.18 (s, 3H), 1.15-1.07 (m, 6H), 1.03 (s, 3H), 0.85 (s, 3H).

Example 51: Synthesis of (1R,2S,5S)—N-[(1S)-1-cyano-2-[(3R)-5,5-dimethyl-2-oxo-pyrrolidin-3-yl]ethyl]-6,6-dimethyl-3-[(2S,3R)-3-(1-methylcyclobutoxy)-2-[(2,2,2-trifluoroacetyl)amino]butanoyl]-3-azabicyclo[3.1.0]hexane-2-carboxamide (Compound 308)

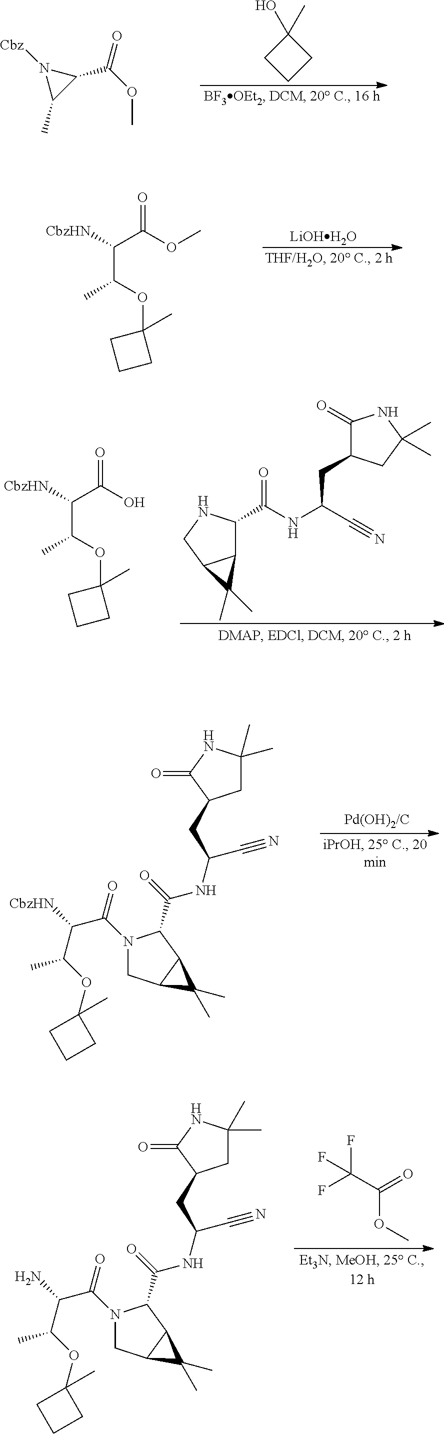

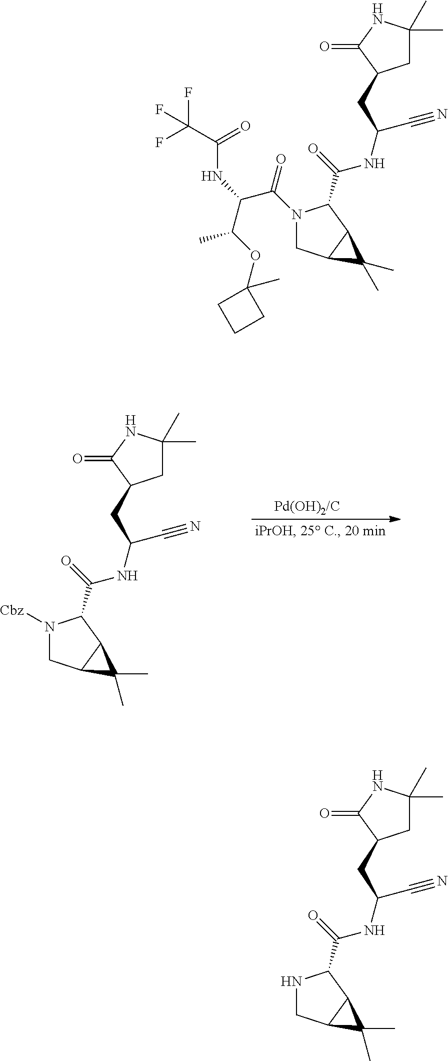 [SMILES: C[C@@H](OC1(C)CCC1)[C@H](NC(=O)C(F)(F)F)C(=O)N1C[C@H]2[C@@H]([C@H]1C(=O)N[C@H](C#N)C[C@@H]1CC(C)(C)NC1=O)C2(C)C ; CC1(C)C[C@@H](C[C@@H](C#N)NC(=O)[C@@H]2[C@@H]3[C@H](CN2C(=O)OCc2ccccc2)C3(C)C)C(=O)N1 ; CC1(C)C[C@@H](C[C@@H](C#N)NC(=O)[C@H]2NC[C@H]3[C@@H]2C3(C)C)C(=O)N1]

Step 1: methyl (3R)-2-(benzyloxycarbonylamino)-3-(1-methylcyclobutoxy)butanoate

To a solution of O1-benzyl O2-methyl (2S,3S)-3-methylaziridine-1,2-dicarboxylate (420 mg, 1.68 mmol, 1 eq) and 1-methylcyclobutanol (174.16 mg, 2.02 mmol, 1.2 eq) in CH₂Cl₂ (10 mL) was added BF₃.Et₂₀ (50.88 mg, 168.50 umol, 44.25 uL, 47% purity, 0.1 eq). The mixture was stirred at 20° C. for 16 h. Upon completion, the reaction mixture was quenched by addition H₂O (50 mL) at 0° C., and then extracted with DCM (30 mL*3). The combined organic layers were washed with brine (40 mL), dried over Na₂SO₄, filtered and concentrated under reduced pressure to give a residue. The residue was purified by prep-TLC (SiO₂, Petroleum ether/Ethyl acetate=4/1) to give methyl (3R)-2-(benzyloxycarbonylamino)-3-(1-methylcyclobutoxy)butanoate (220 mg, 655.95 umol, 38.93% yield) as a yellow oil. MS (ESI) m/z 336.0 [M+H]⁺.

Step 2: (2S,3R)-2-(benzyloxycarbonylamino)-3-(1-methylcyclobutoxy)butanoic acid

A solution of methyl (2S,3R)-2-(benzyloxycarbonylamino)-3-(1-methylcyclobutoxy)butanoate (220.00 mg, 655.95 umol, 1 eq) in THE (6 mL) and H₂O (2 mL) was added LiOH.H₂O (82.58 mg, 1.97 mmol, 3 eq), and the mixture was stirred at 20° C. for 2 h. Upon completion, the reaction mixture was diluted with H₂O (20 mL), adjusted pH=3, and then extracted with ethyl acetate (20 mL*3). The combined organic layers were washed with brine (20 mL), dried over Na₂SO₄, filtered and concentrated under reduced pressure to give (2S,3R)-2-(benzyloxycarbonylamino)-3-(1-methylcyclobutoxy)butanoic acid (200 mg, crude) as a colourless oil. MS (ESI) m/z 322.2 [M+H]⁺.

Step 3: (1R,2S,5S)—N-[(1S)-1-cyano-2-[(3R)-5,5-dimethyl-2-oxo pyrrolidin-3-yl]ethyl]-6,6-dimethyl-3-azabicyclo[3.1.0]hexane-2-carboxamide

To a solution of benzyl (1R,2S,5S)-2-[[(1S)-1-cyano-2-[(3R)-5,5-dimethyl-2-oxo-pyrrolidin-3-yl]ethyl]carbamoyl]-6,6-dimethyl-3-azabicyclo[3.1.0]hexane-3-carboxylate (350 mg, 773.40 umol, 1 eq) in isopropanol (6 mL) was added Pd(OH)₂ (80 mg, 113.93 umol, 20% purity, 1.47e-1 eq) under H₂. The suspension was degassed and purged with H₂ for 3 times. The mixture was stirred under H₂ (15 Psi) at 25° C. for 20 min. Upon completion, the reaction was filtered and concentrated under reduced pressure to give (1R,2S,5S)—N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (230 mg, crude) as a white solid. MS (ESI) m/z 319.3 [M+H]⁺.

Step 4: benzyl N-[(1S,2R)-1-[(1R,2S,5S)-2-[[(1S)-1-cyan-2-[(3R)-5,5-dimethyl-2-oxo-pyrrolidin-3-yl]ethyl]carbamoyl]-6,6-dimethyl-3-azabicyclo[3.1.0]hexane-3-carbonyl]-2-(1-methylcyclobutoxy)propyl]carbamate

To a solution of (2S,3R)-2-(benzyloxycarbonylamino)-3-(1-methylcyclobutoxy)butanoic acid (200.00 mg, 622.34 umol, 1 eq) and (1R,2S,5S)—N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (217.98 mg, 684.57 umol, 1.1 eq) in DCM (6 mL) was added DMAP (152.06 mg, 1.24 mmol, 2 eq) and EDCI (357.91 mg, 1.87 mmol, 3 eq) at 0° C. The mixture was stirred at 20° C. for 2 h. Upon completion, the reaction mixture was quenched by addition H₂O (20 mL), and extracted with DCM (20 mL*3). The combined organic layers were washed with brine (30 mL), dried over Na₂SO₄, filtered and concentrated under reduced pressure to give a residue. The residue was purified by column chromatography (SiO₂, Petroleum ether/Ethyl acetate=10/1 to 1/3) to give benzyl N-[(1S,2R)-1-[(1R,2S,5S)-2-[[(1S)-1-cyano-2-[(3R)-5,5-dimethyl-2-oxo-pyrrolidin-3-yl]ethyl]carbamoyl]-6,6-dimethyl-3-azabicyclo[3.1.0]hexane-3-carbonyl]-2-(1-methylcyclobutoxy)propyl]carbamate (110 mg, 176.92 umol, 28.43% yield) as a yellow oil. MS (ESI) m/z 622.5 [M+H]⁺.

Step 5: (1R,2S,5S)-3-[(2S,3R)-2-amino-3-(1-methylcyclobutoxy)butanoyl]-N-[(1S)-1-cyano-2-[(3R)-5,5-dimethyl-2-oxo pyrrolidin-3-yl]ethyl]-6,6-dimethyl-3-azabicyclo[3.1.0]hexane-2-carboxamide

To a solution of benzyl N-[(1S,2R)-1-[(1R,2S,5S)-2-[[(1S)-1-cyano-2-[(3R)-5,5-dimethyl-2-oxo-pyrrolidin-3-yl]ethyl]carbamoyl]-6,6-dimethyl-3-azabicyclo[3.1.0]hexane-3-carbonyl]-2-(1-methylcyclobutoxy)propyl]carbamate (110.00 mg, 176.92 umol, 1 eq) in isopropanol (6 mL) was added Pd(OH)₂ (250.00 mg, 356.02 umol, 20% purity, 2.01 eq) under N₂ atmosphere. The suspension was degassed and purged with H₂ for 3 times. The mixture was stirred under H₂ (15 Psi) at 25° C. for 15 min. Upon completion, the reaction was filtered and concentrated under reduced pressure to give (1R,2S,5S)-3-[(2S,3R)-2-amino-3-(1-methylcyclobutoxy)butanoyl]-N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (45 mg, crude) was obtained as a colorless oil. MS (ESI) m/z 488.4 [M+H]⁺.

Step 6: (1R,2S,5S)—N-[(1S)-1-cyano-2-[(3R)-5,5-dimethyl-2-oxo pyrrolidin-3-yl]ethyl]-6,6-dimethyl-3-[(2S,3R)-3-(1-methylcyclobutoxy)-2-[(2,2,2-trifluoroacetyl)amino]butanoyl]-3-azabicyclo[3.1.0]hexane-2-carboxamide

To a solution of (1R,2S,5S)-3-[(2S,3R)-2-amino-3-(1-methylcyclobutoxy)butanoyl]-N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (45 mg, 92.28 umol, 1 eq) in MeOH (3 mL) was added drop-wise Et₃N (28.01 mg, 276.85 umol, 38.53 uL, 3 eq), then methyl 2,2,2-trifluoroacetate (118.17 mg, 922.82 umol, 93.05 uL, 10 eq) was added drop-wise, the mixture was stirred at 25° C. for 12 h. Upon completion, the reaction mixture was concentrated under reduced pressure to give a residue. The residue was purified by prep-HPLC (column: Waters Xbridge BEH C18 100*30 mm*10 um; mobile phase: [water (NH₄HCO₃)-ACN]; B %: 35%-65%, 8 min) to give (1R,2S,5S)—N-[(1S)-1-cyano-2-[(3R)-5,5-dimethyl-2-oxo-pyrrolidin-3-yl]ethyl]-6,6-dimethyl-3-[(2S,3R)-3-(1-methylcyclobutoxy)-2-[(2,2,2-trifluoroacetyl)amino]butanoyl]-3-azabicyclo[3.1.0]hexane-2-carboxamide (19.81 mg, 33.94 umol, 36.78% yield, 100% purity) as a white solid. MS (ESI) m/z 584.4 [M+H]⁺.

¹H NMR (400 MHz, DMSO-d₆) δ=9.79 (s, 1H), 8.98 (d, J=8.6 Hz, 1H), 7.80 (s, 1H), 4.92 (ddd, J=5.1, 8.6, 10.6 Hz, 1H), 4.41 (brd, J=8.6 Hz, 1H), 4.11 (s, 1H), 3.96 (dd, J=5.5, 10.4 Hz, 1H), 3.89-3.78 (m, 1H), 3.71-3.63 (m, 1H), 2.63-2.54 (m, 1H), 2.22-2.02 (m, 3H), 1.97 (dd, J=8.6, 12.2 Hz, 1H), 1.79-1.65 (m, 3H), 1.61-1.44 (m, 4H), 1.33 (d, J=7.6 Hz, 1H), 1.30-1.24 (m, 3H), 1.18 (s, 3H), 1.15-1.06 (m, 6H), 1.04-1.01 (m, 3H), 0.84 (s, 3H)

Example 52: Synthesis of (1R,2S,5S)—N-[(1S)-1-cyano-2-[(3R)-5,5-dimethyl-2-oxo-pyrrolidin-3-yl]ethyl]-6,6-dimethyl-3-[(2S,3R)-2-[(2,2,2-trifluoroacetyl)amino]-3-(2,2,2-trifluoroethoxy)butanoyl]-3-azabicyclo[3.1.0]hexane-2-carboxamide (Compound 312)

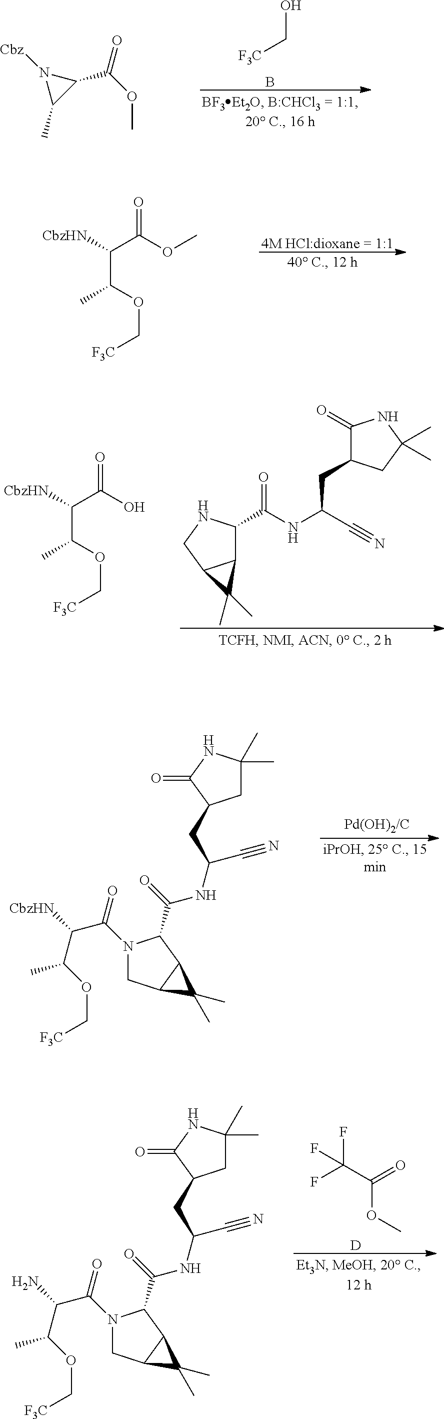

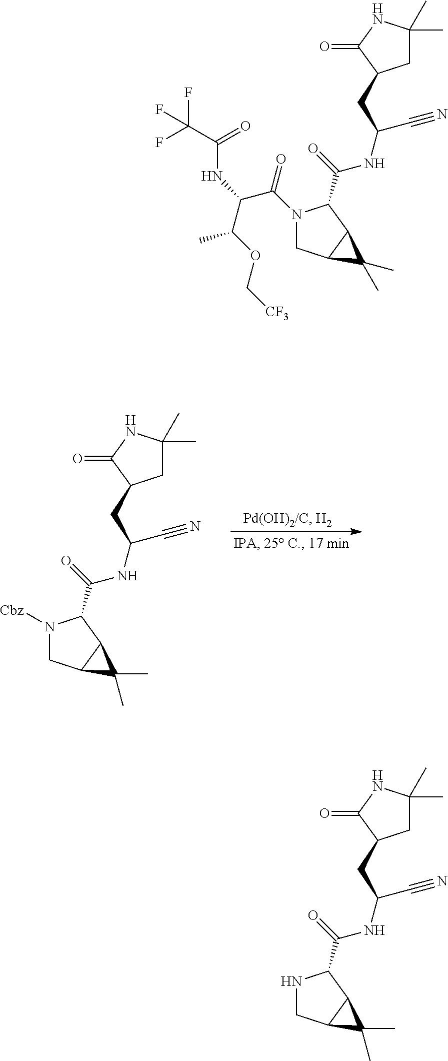 [SMILES: C[C@@H](OCC(F)(F)F)[C@H](NC(=O)C(F)(F)F)C(=O)N1C[C@H]2[C@@H]([C@H]1C(=O)N[C@H](C#N)C[C@@H]1CC(C)(C)NC1=O)C2(C)C ; CC1(C)C[C@@H](C[C@@H](C#N)NC(=O)[C@@H]2[C@@H]3[C@H](CN2C(=O)OCc2ccccc2)C3(C)C)C(=O)N1 ; CC1(C)C[C@@H](C[C@@H](C#N)NC(=O)[C@H]2NC[C@H]3[C@@H]2C3(C)C)C(=O)N1]

Step 1: methyl (2S,3R)-2-(benzyloxycarbonylamino)-3-(2,2,2-trifluoroethoxy)butanoate

To a solution of O1-benzyl O2-methyl (2S,3S)-3-methylaziridine-1,2-dicarboxylate (2 g, 8.02 mmol, 1 eq) and 2,2,2-trifluoroethanol (27.80 g, 277.89 mmol, 20.00 mL, 34.63 eq) in CHCl₃ (20 mL) was added BF₃.Et₂₀ (1.21 g, 4.01 mmol, 1.05 mL, 47% purity, 0.5 eq), and then the mixture was stirred at 20° C. for 16 h. Upon completion, the reaction mixture was quenched by addition H₂O (50 mL) at 0° C. and then extracted with DCM (30 mL*3). The resulting mixture was washed with brine (40 mL), dried over Na₂SO₄, filtered and concentrated under reduced pressure to give a residue. The residue was purified by column chromatography (SiO₂, Petroleum ether:Ethyl acetate=1:0 to 10:1) to give methyl (2S,3R)-2-(benzyloxycarbonylamino)-3-(2,2,2-trifluoroethoxy)butanoate (1 g, 2.86 mmol, 35.68% yield) as a colourless oil. MS (ESI) m/z 350.2 [M+H]⁺.

Step 2: (2S,3R)-2-(benzyloxycarbonylamino)-3-(2,2,2-trifluoroethoxy)butanoic acid

A solution of methyl (2S,3R)-2-(benzyloxycarbonylamino)-3-(2,2,2-trifluoroethoxy)butanoate (25 mg, 71.57 umol, 1 eq) in dioxane (1.5 mL) was added drop-wise with HCl (4 M, 1.5 mL, 83.83 eq), and then the mixture was stirred at 40° C. for 12 h. Upon completion, the reaction mixture was concentrated under reduced pressure to give a residue. The residue was purified by prep-HPLC (column: Phenomenex luna C18 250*50 mm*10 um; mobile phase: [water (HCl)-ACN]; B %: 30%-60%, 10 min) to give (2S,3R)-2-(benzyloxycarbonylamino)-3-(2,2,2-trifluoroethoxy)butanoic acid (450 mg, 1.34 mmol, 46.88% yield) as a yellow solid. MS (ESI) m/z 336.2 [M+H]⁺.

Step 3: (1R,2S,5S)—N-[(1S)-1-cyano-2-[(3R)-5,5-dimethyl-2-oxo pyrrolidin-3-yl]ethyl]-6,6-dimethyl-3-azabicyclo[3.1.0]hexane-2-carboxamide

A solution of benzyl (1R,2S,5S)-2-[[(1S)-1-cyano-2-[(3R)-5,5-dimethyl-2-oxo-pyrrolidin-3-yl]ethyl]carbamoyl]-6,6-dimethyl-3-azabicyclo[3.1.0]hexane-3-carboxylate (450 mg, 994.38 umol, 1 eq) in IPA (2 mL) was added Pd(OH)₂ (150 mg, 213.62 umol, 20% purity, 2.15e-1 eq) under H₂ (15 Psi), and then the mixture was stirred at 25° C. for 17 min. Upon completion, the reaction mixture was filtered and the filtrate was concentrated under reduced pressure to give (1R,2S,5S)—N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (300 mg, crude) as a white solid. MS (ESI) m/z 319.3 [M+H]⁺.

Step 4: benzyl N-[(1S,2R)-1-[(1R,2S,5S)-2-[[(1S)-1-cyan-2-[(3R)-5,5-dimethyl-2-oxo-pyrrolidin-3-yl]ethyl]carbamoyl]-6,6-dimethyl-3-azabicyclo[3.1.0]hexane-3-carbonyl]-2 (2,2,2-trifluoroethoxy)propyl]carbamate

To a solution of (2S,3R)-2-(benzyloxycarbonylamino)-3-(2,2,2-trifluoroethoxy)butanoic acid (200 mg, 596.53 umol, 1 eq) and (1R,2S,5S)—N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (284.91 mg, 894.79 umol, 1.5 eq) in acetonitrile (10 mL) was cooled to 0° C. 1-Methylimidazole (195.91 mg, 2.39 mmol, 190.20 uL, 4 eq) and TCFH (200.85 mg, 715.83 umol, 1.2 eq) were added at 0° C. The resulting mixture was stirred at 0° C. for 2 h. Upon completion, the reaction mixture was diluted with H₂O (30 mL) and extracted with DCM (20 mL*3). The combined organic layers were dried over Na₂SO₄, filtered and concentrated under reduced pressure to give a residue. The residue was purified by prep-TLC (SiO₂, Petroleum ether:Ethyl acetate=0:1) to give benzyl N-[(1S,2R)-1-[(1R,2S,5S)-2-[[(1S)-1-cyano-2-[(3R)-5,5-dimethyl-2-oxo-pyrrolidin-3-yl]ethyl]carbamoyl]-6,6-dimethyl-3-azabicyclo[3.1.0]hexane-3-carbonyl]-2-(2,2,2-trifluoroethoxy)propyl]carbamate (240 mg, 377.55 umol, 63.29% yield) as a white solid. MS (ESI) m/z 636.5 [M+H]⁺.

Step 5: (1R,2S,5S)-3-[(2S,3R)-2-amino-3-(2,2,2-trifluoroethoxy)butanoyl]-N-[(1S)-1-cyano-2-[(3R)-5,5-dimethyl-2-oxo pyrrolidin-3-yl]ethyl]-6,6-dimethyl-3-azabicyclo[3.1.0]hexane-2-carboxamide

A solution of benzyl N-[(1S,2R)-1-[(1R,2S,5S)-2-[[(1S)-1-cyano-2-[(3R)-5,5-dimethyl-2-oxo-pyrrolidin-3-yl]ethyl]carbamoyl]-6,6-dimethyl-3-azabicyclo[3.1.0]hexane-3-carbonyl]-2-(2,2,2-trifluoroethoxy)propyl]carbamate (240 mg, 377.55 umol, 1 eq) in isopropanol (10 mL) was added Pd(OH)₂ (240.00 mg, 341.78 umol, 20% purity, 9.05e-1 eq) under H₂ (15 Psi), the mixture was stirred at 25° C. for 15 min. Upon completion, the reaction mixture was filtered and the filtrate was concentrated under reduced pressure to give (1R,2S,5S)-3-[(2S,3R)-2-amino-3-(2,2,2-trifluoroethoxy)butanoyl]-N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (160 mg, crude) as a white solid.

Step 6: (1R,2S,5S)—N-[(1S)-1-cyano-2-[(3R)-5,5-dimethyl-2-oxo pyrrolidin-3-yl]ethyl]-6,6-dimethyl-3-[(2S,3R)-2-[(2,2,2-trifluoroacetyl)amino]-3-(2,2,2-trifluoroethoxy)butanoyl]-3-azabicyclo[3.1.0]hexane-2-carboxamide

A mixture of (1R,2S,5S)-3-[(2S,3R)-2-amino-3-(2,2,2-trifluoroethoxy)butanoyl]-N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (160 mg, 319.02 umol, 1 eq) in MeOH (10 mL) was added Et₃N (96.84 mg, 957.05 umol, 133.21 uL, 3 eq) and methyl 2,2,2-trifluoroacetate (4.45 g, 34.71 mmol, 3.5 mL, 108.81 eq). The resulting mixture was stirred at 20° C. for 12 h.

Upon completion, the reaction mixture was concentrated under reduced pressure to give a residue. The residue was purified by prep-HPLC (column: Waters Xbridge Prep OBD C18 150*40 mm*10 um; mobile phase: [water (NH₄HCO₃)-ACN]; B %: 30%-70%, 8 min) to give (1R,2S,5S)—N-[(1S)-1-cyano-2-[(3R)-5,5-dimethyl-2-oxo-pyrrolidin-3-yl]ethyl]-6,6-dimethyl-3-[(2S,3R)-2-[(2,2,2-trifluoroacetyl)amino]-3-(2,2,2-trifluoroethoxy)butanoyl]-3-azabicyclo[3.1.0]hexane-2-carboxamide (66 mg, 110.45 umol, 34.62% yield, 100% purity) as a white solid. MS (ESI) m/z 598.2 [M+H]⁺.

¹H NMR (400 MHz, DMSO-d₆) δ=9.96 (br s, 1H), 9.02 (d, J=8.3 Hz, 1H), 7.81 (s, 1H), 5.00-4.64 (m, 1H), 4.47 (br d, J=7.5 Hz, 1H), 4.19-4.06 (m, 2H), 4.06-3.98 (m, 1H), 3.98-3.92 (m, 1H), 3.91-3.84 (m, 1H), 3.81 (d, J=10.5 Hz, 1H), 2.60-2.52 (m, 1H), 2.28-2.08 (m, 1H), 2.03-1.91 (m, 1H), 1.83-1.67 (m, 1H), 1.60-1.42 (m, 2H), 1.33 (d, J=7.5 Hz, 1H), 1.28-1.15 (m, 6H), 1.13-0.99 (m, 6H), 0.86 (s, 3H).

Example 53: Synthesis of (1R,2S,5S)—N-[(1S)-1-cyan-2-[(6R)-5-oxo-4-azaspiro[2.4]heptan-6-yl]ethyl]-6,6-dimethyl-3-[(2S,3R)-3-(1-methylcyclobutoxy)-2-[(2,2,2-trifluoroacetyl)amino]butanoyl]-3-azabicyclo[3.1.0]hexane-2-carboxamide (Compound 314)

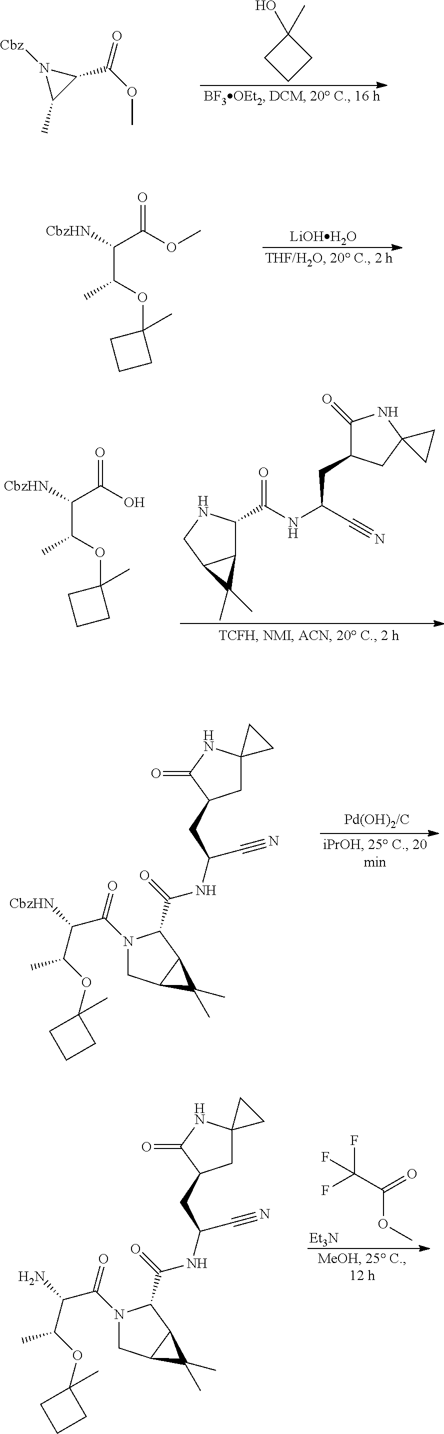

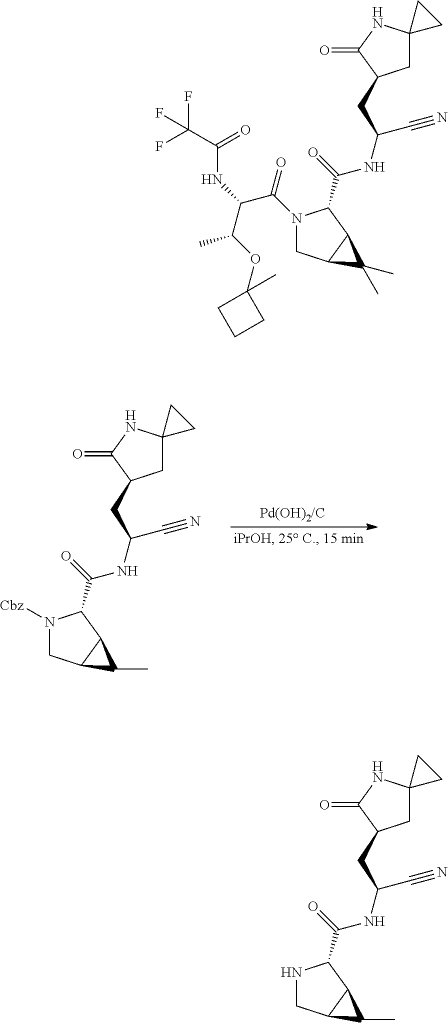

Step 1: methyl (2S,3R)-2-(benzyloxycarbonylamino)-3-(1-methylcyclobutoxy)butanoate

To a solution of O1-benzyl O2-methyl (2S,3S)-3-methylaziridine-1,2-dicarboxylate (1.6 g, 6.42 mmol, 1 eq) and 1-methylcyclobutanol (663.45 mg, 7.70 mmol, 1.2 eq) in DCM (30 mL) was added BF₃.Et₂₀ (193.84 mg, 641.90 umol, 168.55 uL, 47% purity, 0.1 eq). The mixture was stirred at 20° C. for 16 h. Upon completion, the reaction mixture was quenched by addition H₂O (30 mL), and then extracted with DCM (30 mL*3). The combined organic layers were washed with and brine (60 mL), dried over Na₂SO₄, filtered and concentrated under reduced pressure to give a residue. The residue was purified by column chromatography (SiO₂, Petroleum ether/Ethyl acetate=I/O to 10/1) to give methyl (3R)-2-(benzyloxycarbonylamino)-3-(1-methylcyclobutoxy)butanoate (1 g, 2.98 mmol, 46.45% yield) as a colorless oil. MS (ESI) m/z 336.3 [M+H]⁺.

Step 2: (2S,3R)-2-(benzyloxycarbonylamino)-3-(1-methylcyclobutoxy)butanoic acid

A solution of methyl (2S,3R)-2-(benzyloxycarbonylamino)-3-(1-methylcyclobutoxy)butanoate (1 g, 2.98 mmol, 1 eq) in THE (15 mL) and H₂O (5 mL) was added LiOH.H₂O (375.35 mg, 8.94 mmol, 3 eq), the mixture was stiffed at 20° C. for 2 h. Upon completion, the reaction mixture was diluted with H₂O (30 mL) and adjusted PH=3, then was extracted with EA (30 mL*3). The combined organic layers were washed with brine (30 mL), dried over Na₂SO₄, filtered and concentrated under reduced pressure to give (2S,3R)-2-(benzyloxycarbonylamino)-3-(1-methylcyclobutoxy)butanoic acid (750 mg, crude) as a colourless oil. MS (ESI) m/z 322.3 [M+H]⁺.

Step 3: (1R,2S,5S)—N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide

To a solution of benzyl (1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carboxylate (1.5 g, 3.33 mmol, 1 eq) in IPA (20 mL) was added Pd(OH)₂ (508.53 mg, 20% purity,) under H₂. The suspension was degassed and purged with H₂ for 3 times. The mixture was stirred under H₂ (15 Psi) at 25° C. for 15 min. Upon completion, the reaction was filtered and concentrated under reduced pressure to give (1R,2S,5S)—N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (900 mg, crude) as a white solid. MS (ESI) m/z 317.2 [M+H]⁺.

Step 4: benzyl N-[(1S,2R)-1-[(1R,2S,5S)-2-[[(1S)-1-cyan-2-[(6R)-5-oxo-4-azaspiro[2.4]heptan-6-yl]ethyl]carbamoyl]-6,6-dimethyl-3-azabicyclo[3.1.0]hexane-3-carbonyl]-2-(1-methylcyclobutoxy)propyl]carbamate

To a solution of (2S,3R)-2-(benzyloxycarbonylamino)-3-(1-methylcyclobutoxy)butanoic acid (700 mg, 2.18 mmol, 1 eq) and (1R,2S,5S)—N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (827.01 mg, 2.61 mmol, 1.2 eq) in MeCN (20 mL) was added 1-methylimidazole (536.49 mg, 6.53 mmol, 520.86 uL, 3 eq) and TCFH (1.22 g, 4.36 mmol, 2 eq), and then the mixture was stirred at 20° C. for 2 h. Upon completion, the reaction mixture was quenched by addition H₂O (30 mL), and extracted with DCM (30 mL*3). The combined organic layers were washed with brine (60 mL), dried over Na₂SO₄, filtered and concentrated under reduced pressure to give a residue. The residue was purified by column chromatography (SiO₂, Petroleum ether/Ethyl acetate=10/1 to 1/3) to give benzyl N-[(1S,2R)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2-(1-methylcyclobutoxy)propyl]carbamate (620 mg, 1.00 mmol, 45.93% yield) as a white solid. MS (ESI) m/z 620.5 [M+H]⁺.

Step 5: (1R,2S,5S)-3-[(2S,3R)-2-amino-3-(1-methylcyclobutoxy)butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide

To a solution of benzyl N-[(1S,2R)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2-(1-methylcyclobutoxy)propyl]carbamate (600 mg, 968.13 umol, 1 eq) in i-PrOH (20 mL) was added Pd(OH)₂ (600 mg, 20% purity) under N₂ atmosphere. The suspension was degassed and purged with H₂ for 3 times. The mixture was stirred under H₂ (15 Psi) at 25° C. for 15 min. Upon completion, the reaction was filtered and concentrated under reduced pressure to give (1R,2S,5S)-3-[(2S,3R)-2-amino-3-(1-methylcyclobutoxy)butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (400 mg, crude) as a white solid. MS (ESI) m/z 486.4 [M+H]⁺.

Step 6: (1R,2S,5S)—N-[(1S)-1-cyano-2-[(6R)-5-oxo-4-azaspiro[2.4]heptan-6-yl]ethyl]-6,6-dimethyl-3-[(2S,3R)-3-(1-methylcyclobutoxy)-2-[(2,2,2-trifluoroacetyl)amino]butanoyl]-3-azabicyclo[3.1.0]hexane-2-carboxamide

To a solution of (1R,2S,5S)-3-[(2S,3R)-2-amino-3-(1-methylcyclobutoxy)butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (400 mg, 823.69 umol, 1 eq) in MeOH (10 mL) was added drop-wise Et₃N (250.05 mg, 2.47 mmol, 343.95 uL, 3 eq). Then, methyl 2,2,2-trifluoroacetate (1.05 g, 8.24 mmol, 830.50 uL, 10 eq) was added drop-wise, and the resulting mixture was stirred at 25° C. for 12 h. Upon completion, the reaction mixture was concentrated under reduced pressure to give a residue. The residue was purified by prep-HPLC (column: Waters Xbridge Prep OBD C18 150*40 mm*10 um; mobile phase: [water (NH₄HCO₃)-ACN]; B %: 40%-60%, 8 min) to give (1R,2S,5S)—N-[(1S)-1-cyano-2-[(6R)-5-oxo-4-azaspiro[2.4]heptan-6-yl]ethyl]-6,6-dimethyl-3-[(2S,3R)-3-(1-methylcyclobutoxy)-2-[(2,2,2-trifluoroacetyl)amino]butanoyl]-3-azabicyclo[3.1.0]hexane-2-carboxamide (90 mg, 154.74 umol, 18.79% yield, 100% purity) as a white solid. MS (ESI) m/z 582.4 [M+H]⁺.

1H NMR (400 MHz, DMSO-d₆) δ=9.81 (s, 1H), 9.00 (d, J=8.3 Hz, 1H), 7.77 (s, 1H), 4.95-4.91 (m, 1H), 4.41 (br d, J=7.4 Hz, 1H), 4.13 (s, 1H), 3.96 (br dd, J=5.5, 10.4 Hz, 1H), 3.91-3.80 (m, 1H), 3.66 (br d, J=10.5 Hz, 1H), 2.64 (br d, J=4.1 Hz, 1H), 2.21-2.05 (m, 3H), 2.01-1.87 (m, 2H), 1.83-1.68 (m, 3H), 1.59-1.45 (m, 3H), 1.44-1.44 (m, 1H), 1.27 (s, 3H), 1.08 (d, J=6.0 Hz, 3H), 1.03 (s, 3H), 0.84 (s, 3H), 0.70-0.69 (m, 1H), 0.62-0.49 (m, 3H).

Example 54: Synthesis of (1R,2S,5S)—N-[(1S)-1-cyan-2-[(6R)-5-oxo-4-azaspiro[2.4]heptan-6-yl]ethyl]-6,6-dimethyl-3-[(2S,3R)-3-(1-methylcyclopropoxy)-2-[(2,2,2-trifluoroacetyl)amino]butanoyl]-3-azabicyclo[3.1.0]hexane-2-carboxamide (Compound 315)

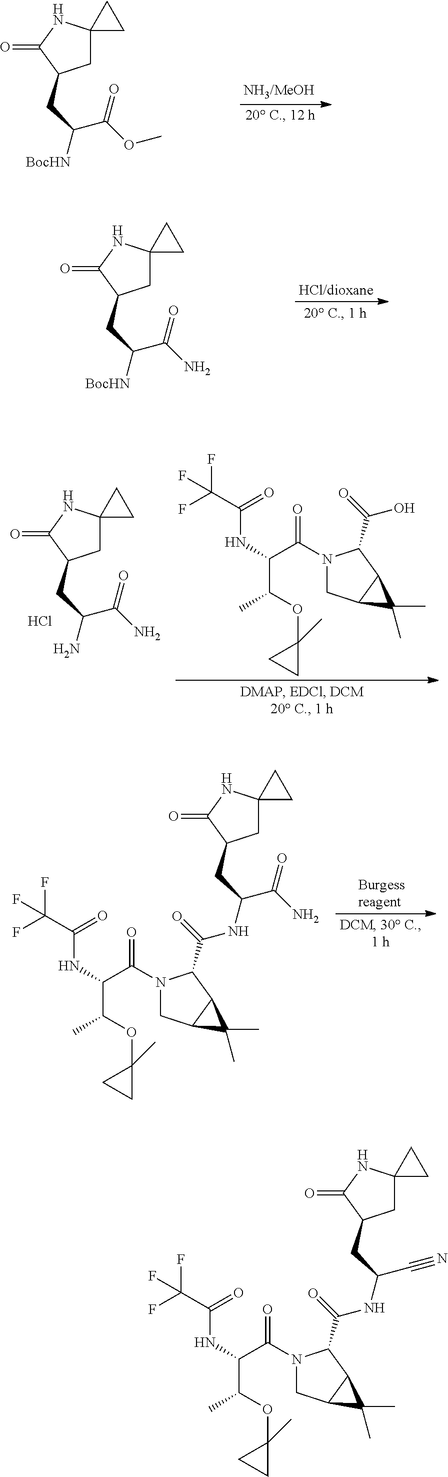

Step 1: tert-butyl N-[(1S)-2-amino-2-oxo-1-[[(6R)-5-oxo-4-azaspiro[2.4]heptan-6-yl]methyl]ethyl]carbamate

A mixture of methyl (2S)-2-(tert-butoxycarbonylamino)-3-[(6R)-5-oxo-4-azaspiro[2.4]heptan-6-yl]propanoate (3 g, 9.60 mmol, 1 eq) in NH₃/MeOH (7 M, 60.00 mL, 43.73 eq) was stirred at 20° C. for 12 h. Upon completion, the reaction mixture was concentrated under reduced pressure to give tert-butyl N-[(1S)-2-amino-2-oxo-1-[[(6R)-5-oxo-4-azaspiro[2.4]heptan-6-yl]methyl]ethyl]carbamate (3 g, crude) as a yellow solid. MS (ESI) m/z 298.2 [M+H]⁺.

Step 2: (2S)-2-amino-3-[(6R)-5-oxo-4-azaspiro[2.4]heptan-6-yl]propanamide

A mixture of tert-butyl N-[(1S)-2-amino-2-oxo-1-[[(6R)-5-oxo-4-azaspiro[2.4]heptan-6-yl]methyl]ethyl]carbamate (300 mg, 1.01 mmol, 1 eq) in HCl/dioxane (4 M, 3.00 mL, 11.89 eq) was stirred at 20° C. for 1 h. Upon completion, the reaction mixture was concentrated under reduced pressure to give (2S)-2-amino-3-[(6R)-5-oxo-4-azaspiro[2.4]heptan-6-yl]propanamide (240 mg, crude, HCl) as a white solid. MS (ESI) m/z 198.2 [M+H]⁺.

Step 3: (1R,2S,5S)—N-[(1S)-2-amino-2-oxo-1-[[(6R)-5-oxo-4-azaspiro[2.4]heptan-6-yl]methyl]ethyl]-6,6-dimethyl-3-[(2S,3R)-3-(1-methylcyclopropoxy)-2-[(2,2,2-trifluoroacetyl)amino]butanoyl]-3-azabicyclo[3.1.0]hexane-2-carboxamide

To a solution of (2S)-2-amino-3-[(6R)-5-oxo-4-azaspiro[2.4]heptan-6-yl]propanamide (240 mg, 1.03 mmol, 1 eq, HCl), (1R,2S,5S)-6,6-dimethyl-3-[(2S,3R)-3-(1-methylcyclopropoxy)-2-[(2,2,2-trifluoroacetyl)amino]butanoyl]-3-azabicyclo[3.1.0]hexane-2-carboxylic acid (417.36 mg, 1.03 mmol, 1 eq), DMAP (313.66 mg, 2.57 mmol, 2.5 eq) in DCM (5 mL) was added EDCI (590.62 mg, 3.08 mmol, 3 eq). The mixture was stirred at 20° C. for 1 h. Upon completion, the reaction mixture was poured into H₂O (30 mL), and then extracted with DCM (30 mL*3). The combined organic layers were washed with brine (30 mL), dried over Na₂SO₄, filtered and concentrated under reduced pressure to give a residue. The residue was purified by column chromatography (SiO₂, Petroleum ether:Ethyl acetate=1:7 to 0:1) to give (1R,2S,5S)—N-[(1S)-2-amino-2-oxo-1-[[(6R)-5-oxo-4-azaspiro[2.4]heptan-6-yl]methyl]ethyl]-6,6-dimethyl-3-[(2S,3R)-3-(1-methylcyclopropoxy)-2-[(2,2,2-trifluoroacetyl)amino]butanoyl]-3-azabicyclo[3.1.0]hexane-2-carboxamide (570 mg, 573.30 umol, 55.82% yield, 58.9% purity) as a yellow solid. MS (ESI) m/z 586.3 [M+H]⁺.

Step 4: (1R,2S,5S)—N-[(1S)-1-cyano-2-[(6R)-5-oxo-4-azaspiro[2.4]heptan-6-yl]ethyl]-6,6-dimethyl-3-[(2S,3R)-3-(1-methylcyclopropoxy)-2-[(2,2,2-trifluoroacetyl)amino]butanoyl]-3-azabicyclo[3.1.0]hexane-2-carboxamide

To a solution of (1R,2S,5S)—N-[(1S)-2-amino-2-oxo-1-[[(6R)-5-oxo-4-azaspiro[2.4]heptan-6-yl]methyl]ethyl]-6,6-dimethyl-3-[(2S,3R)-3-(1-methylcyclopropoxy)-2-[(2,2,2-trifluoroacetyl)amino]butanoyl]-3-azabicyclo[3.1.0]hexane-2-carboxamide (560 mg, 956.26 umol, 1 eq) in DCM (5 mL) was added Burgess reagent (683.65 mg, 2.87 mmol, 3 eq). The mixture was stirred at 30° C. for 1 h. Upon completion, the reaction mixture was diluted with H₂O (30 mL) and celite filtered then extracted with EtOAc (30 mL*3). The combined organic layers were dried over Na₂SO₄, filtered and concentrated under reduced pressure to give a residue. The residue was purified by prep-HPLC(column: Waters Xbridge Prep OBD C18 150*40 mm*10 um; mobile phase: [water (NH₄HCO₃)-ACN]; B %: 35% 65%, 8 min) to give (1R,2S,5S)—N-[(1S)-1-cyano-2-[(6R)-5-oxo-4-azaspiro[2.4]heptan-6-yl]ethyl]-6,6-dimethyl-3-[(2S,3R)-3-(1-methylcyclopropoxy)-2-[(2,2,2-trifluoroacetyl)amino]butanoyl]-3-azabicyclo[3.1.0]hexane-2-carboxamide (114.17 mg, 201.15 umol, 21.03% yield, 100% purity) as a white solid. MS (ESI) m/z 584.3 [M+H]⁺.

¹H NMR (400 MHz, DMSO-d₆) δ=9.92 (d, J=7.2 Hz, 1H), 9.03 (d, J=8.6 Hz, 1H), 7.78 (s, 1H), 4.94 (ddd, J=5.5, 8.6, 10.5 Hz, 1H), 4.34 (t, J=7.7 Hz, 1H), 4.12 (s, 1H), 3.94-3.89 (m, 2H), 3.67 (d, J=10.3 Hz, 1H), 2.71-2.61 (m, 1H), 2.24-2.11 (m, 1H), 2.03-1.87 (m, 2H), 1.81 (ddd, J=5.5, 10.6, 13.5 Hz, 1H), 1.56 (dd, J=5.5, 7.5 Hz, 1H), 1.34 (s, 1H), 1.32 (s, 3H), 1.15 (d, J=6.1 Hz, 3H), 1.02 (s, 3H), 0.83 (s, 3H), 0.80-0.74 (m, 2H), 0.67-0.55 (m, 4H), 0.38-0.30 (m, 2H).

Example 55: Synthesis of (1R,2S,5S)—N-[(1S)-1-cyano-2-[(3R)-5,5-dimethyl-2-oxo-pyrrolidin-3-yl]ethyl]-6,6-dimethyl-3-[1-[(2,2,2-trifluoroacetyl)amino]cyclobutanecarbonyl]-3-azabicyclo[3.1.0]hexane-2-carboxamide (Compound 316)

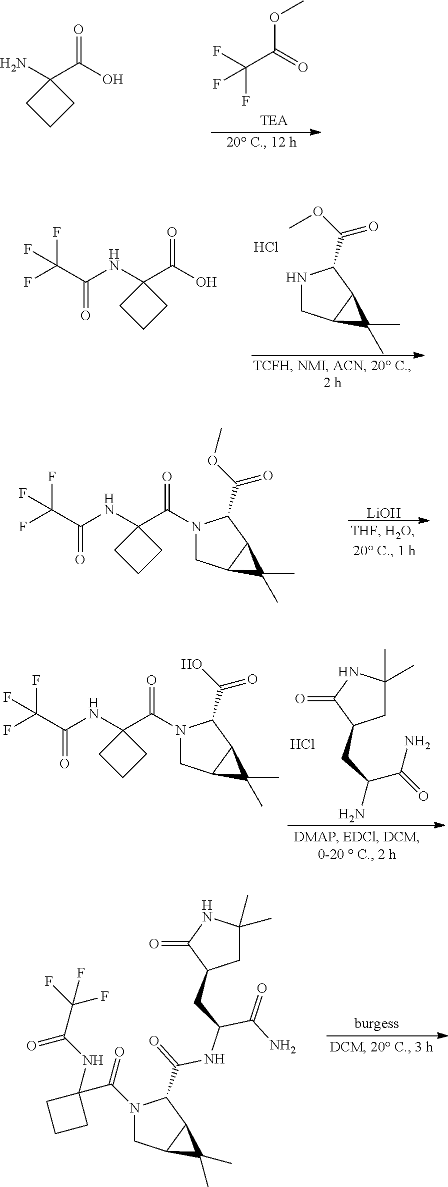

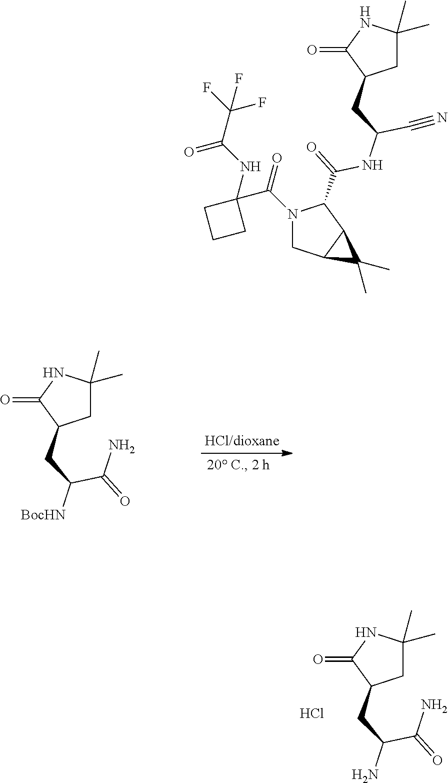 [SMILES: CC1(C)C[C@@H](C[C@@H](C#N)NC(=O)[C@@H]2[C@@H]3[C@H](CN2C(=O)C2(NC(=O)C(F)(F)F)CCC2)C3(C)C)C(=O)N1 ; CC1(C)C[C@@H](C[C@H](NC(=O)OC(C)(C)C)C(N)=O)C(=O)N1 ; Cl ; CC1(C)C[C@@H](C[C@H](N)C(N)=O)C(=O)N1]

Step 1: (2S)-2-amino-3-[(3R)-5,5-dimethyl-2-oxo pyrrolidin-3-yl]propanamide

A solution of tert-butyl N-[(1S)-2-amino-1-[[(3R)-5,5-dimethyl-2-oxo-pyrrolidin-3-yl]methyl]-2-oxo-ethyl]carbamate (1 g, 3.01 mmol, 90% purity, 1 eq) in HCl/dioxane (10 mL) was stirred at 20° C. for 2 h. Upon completion, the reaction mixture was concentrated under reduced pressure to give (2S)-2-amino-3-[(3R)-5,5-dimethyl-2-oxo-pyrrolidin-3-yl]propanamide (550 mg, crude) as a yellow solid.

Step 2: 1-[(2,2,2-trifluoroacetyl)amino]cyclobutanecarboxylic acid

To a solution of 1-aminocyclobutanecarboxylic acid (5 g, 43.43 mmol, 1 eq) in methyl 2,2,2-trifluoroacetate (55.61 g, 434.29 mmol, 43.79 mL, 10 eq) was added TEA (13.18 g, 130.28 mmol, 18.13 mL, 3 eq). The mixture was stirred at 20° C. for 12 h. Upon completion, the reaction mixture was concentrated under reduced pressure to afford 1-[(2,2,2-trifluoroacetyl)amino]cyclobutanecarboxylic acid (7 g, crude) as yellow oil. MS (ESI) m/z 209.9 [M+H]⁺.

Step 3: methyl (1R,2S,5S)-6,6-dimethyl-3-[1-[(2,2,2-trifluoroacetyl)amino]cyclobutanecarbonyl]-3-azabicyclo[3.1.0]hexane-2-carboxylate

A mixture of 1-[(2,2,2-trifluoroacetyl)amino]cyclobutanecarboxylic acid (6.9 g, 32.68 mmol, 1 eq), methyl (1R,2S,5S)-6,6-dimethyl-3-azabicyclo[3.1.0]hexane-2-carboxylate (5.53 g, 32.68 mmol, 1 eq), TCFH (13.75 g, 49.02 mmol, 1.5 eq), and 1-methylimidazole (8.05 g, 98.04 mmol, 7.82 mL, 3 eq) in ACN (70 mL) was degassed and purged with N₂ for 3 times, and then the mixture was stirred at 20° C. for 2 h under N₂ atmosphere. Upon completion, the reaction mixture was quenched by addition H₂O (20 mL) at 25° C., and extracted with ethyl acetate (50 mL*3). The combined organic layers were washed with saturated salt water (20 mL*2), dried over Na₂SO₄, filtered and concentrated under reduced pressure to give a residue. The residue was purified by column chromatography (SiO₂, Petroleum ether:Ethyl acetate=1:0 to 1:3) to afford methyl (1R,2S,5S)-6,6-dimethyl-3-[1-[(2,2,2-trifluoroacetyl)amino]cyclobutanecarbonyl]-3-azabicyclo[3.1.0]hexane-2-carboxylate (8 g, 11.04 mmol, 33.78% yield, 50% purity) as yellow solid. MS (ESI) m/z 363.2 [M+H]⁺.

Step 4: (1R,2S,5S)-6,6-dimethyl-3-[1-[(2,2,2-trifluoroacetyl)amino]cyclobutanecarbonyl]-3-azabicyclo[3.1.0]hexane-2-carboxylic acid

To a solution of methyl (1R,2S,5S)-6,6-dimethyl-3-[1-[(2,2,2-trifluoroacetyl)amino]cyclobutanecarbonyl]-3-azabicyclo[3.1.0]hexane-2-carboxylate (7 g, 15.45 mmol, 80% purity, 1 eq) in THE (9 mL) and H₂O (3 mL) was added LiOH.H₂O (1.95 g, 46.36 mmol, 3 eq) and stirred at 20° C. for 1 h. Upon completion, the reaction mixture was added HCl (1M, 10 mL) at 0° C., and then extracted with EA (10 mL*3). The combined organic layers were washed with brine (2 mL), dried over Na₂SO₄, filtered and concentrated under reduced pressure to give (1R,2S,5S)-6,6-dimethyl-3-[1-[(2,2,2-trifluoroacetyl)amino]cyclobutanecarbonyl]-3-azabicyclo[3.1.02hexane-2-carboxylic acid (4 g, crude) as a yellow solid. MS (ESI) m/z 349.1 [M+H]⁺.

Step 5: (1R,2S,5S)—N-[(1S)-2-amino-1-[[(3R)-5,5-dimethyl-2-oxo pyrrolidin-3-yl]methyl]-2-oxo-ethyl]-6,6-dimethyl-3-[1-[(2,2,2-trifluoroacetyl)amino]cyclobutanecarbonyl]-3-azabicyclo[3.1.0]hexane-2-carboxamide

A solution of (1R,2S,5S)-6,6-dimethyl-3-[1-[(2,2,2-trifluoroacetyl)amino]cyclobutanecarbonyl]-3-azabicyclo[3.1.o]hexane-2-carboxylic acid (2 g, 3.45 mmol, 60% purity, 1.25 eq) in DCM (10 mL) was added (2S)-2-amino-3-[(3R)-5,5-dimethyl-2-oxo-pyrrolidin-3-yl]propanamide (550 mg, 2.33 mmol, 6.77e-1 eq, HCl), DMAP (1.01 g, 8.27 mmol, 3 eq) and EDCI (1.06 g, 5.51 mmol, 2 eq) at 0° C. The resulting mixture was stirred at 20° C. for 2 h. Upon completion, the solution was added with brine (100 mL), then was extracted with EA (50 mL*2), the combined organic phase was washed with brine (50 mL), dried over Na₂SO₄, concentrated in vacuum. The residue was purified by prep-TLC (SiO₂, EA, Rf=0.5, UV 254) to afford (1R,2S,5S)—N-[(1S)-2-amino-1-[[(3R)-5,5-dimethyl-2-oxo-pyrrolidin-3-yl]methyl]-2-oxo-ethyl]-6,6-dimethyl-3-[1-[(2,2,2-trifluoroacetyl)amino]cyclobutanecarbonyl]-3-azabicyclo[3.1.0]hexane-2-carboxamide (600 mg, 1.13 mmol, 32.89% yield) as a yellow solid. MS (ESI) m/z 530.4 [M+H]⁺.

Step 6: (1R,2S,5S)—N-[(1S)-1-cyano-2-[(3R)-5,5-dimethyl-2-oxo pyrrolidin-3-yl]ethyl]-6,6-dimethyl-3-[1-[(2,2,2-trifluoroacetyl)amino]cyclobutanecarbonyl]-3-azabicyclo[3.1.0]hexane-2-carboxamide

A mixture of (1R,2S,5S)—N-[(1S)-2-amino-1-[[(3R)-5,5-dimethyl-2-oxo-pyrrolidin-3-yl]methyl]-2-oxo-ethyl]-6,6-dimethyl-3-[1-[(2,2,2-trifluoroacetyl)amino]cyclobutanecarbonyl]-3-azabicyclo[3.1.02hexane-2-carboxamide (600 mg, 1.13 mmol, 1 eq) in DCM (6 mL) was added Burgess reagent (810.04 mg, 3.40 mmol, 3 eq) and stirred at 20° C. for 3 h. Upon completion, the reaction mixture was quenched with water (0.5 mL) and concentrated under reduced pressure to give a residue ((30° C.). The residue was purified by prep-HPLC (column: Phenomenex C18 80*40 mm*3 um; mobile phase: [water(NH₄HCO₃)-ACN]; B %: 20%-40%, 8 min) to give (1R,2S,5S)—N-[(1S)-1-cyano-2-[(3R)-5,5-dimethyl-2-oxo-pyrrolidin-3-yl]ethyl]-6,6-dimethyl-3-[1-[(2,2,2-trifluoroacetyl)amino]cyclobutanecarbonyl]-3-azabicyclo[3.1.0]hexane-2-carboxamide (104.34 mg, 203.97 umol, 18.00% yield, 100% purity) as a white solid. MS (ESI) m/z 512.2 [M+H]⁺.

¹H NMR (400 MHz, DMSO-d₆) δ=8.85 (d, J=8.6 Hz, 1H), 7.83 (s, 1H), 4.98 4.87 (m, 1H), 4.11 (s, 1H), 3.56-3.48 (m, 1H), 3.47-3.38 (m, 1H), 2.78-2.61 (m, 2H), 2.42 (br t, J=7.9 Hz, 2H), 2.22-2.12 (m, 1H), 2.09-1.94 (m, 2H), 1.93-1.81 (m, 1H), 1.79 1.69 (m, 1H), 1.68-1.56 (m, 1H), 1.56-1.46 (m, 2H), 1.23 (d, J=7.5 Hz, 1H), 1.18 (s, 3H), 1.14 (s, 3H), 0.99 (s, 3H), 0.78 (s, 3H).

Example 56: Synthesis of (1R,2S,5S)—N-[(1S)-1-cyan-2-[(6R)-5-oxo-4-azaspiro[2.4]heptan-6-yl]ethyl]-6,6-dimethyl-3-[(2S,3R)-2-[(2,2,2-trifluoroacetyl)amino]-3-(2,2,2-trifluoroethoxy)butanoyl]-3-azabicyclo[3.1.0]hexane-2-carboxamide (Compound 319)

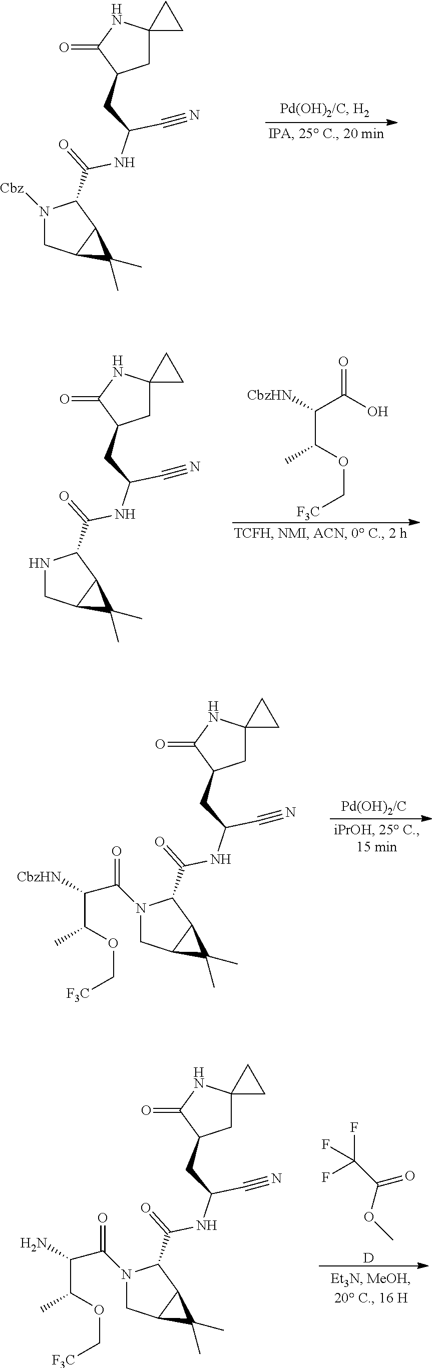

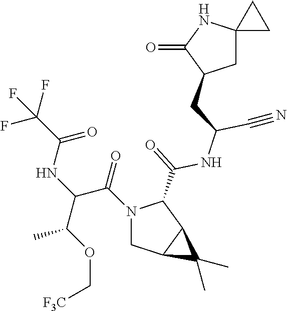 [SMILES: C[C@@H](OCC(F)(F)F)C(NC(=O)C(F)(F)F)C(=O)N1C[C@H]2[C@@H]([C@H]1C(=O)N[C@H](C#N)C[C@@H]1CC3(CC3)NC1=O)C2(C)C]

Step 1: (1R,2S,5S)—N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide

A solution of benzyl (1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carboxylate (350 mg, 776.86 umol, 1 eq) in IPA (10 mL) was added Pd(OH)₂ (350.00 mg, 498.43 umol, 20% purity, 6.42e-1 eq) under H₂ (15 Psi), the mixture was stirred at 25° C. for 20 min. Upon completion, the reaction mixture was filtered and the filtrate was concentrated under reduced pressure to give (1R,2S,5S)—N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (220 mg, crude) as a white solid. MS (ESI) m/z 317.2 [M+H]⁺.

Step 2: benzyl N-[(1S,2R)-1-[(1R,2S,5S)-2-[[(1S)-1-cyan-2-[(6R)-5-oxo-4-azaspiro[2.4]heptan-6-yl]ethyl]carbamoyl]-6,6-dimethyl-3-azabicyclo[3.1.0]hexane-3-carbonyl]-2-(2,2,2-trifluoroethoxy)propyl]carbamate

To a solution of (2S,3R)-2-(benzyloxycarbonylamino)-3-(2,2,2-trifluoroethoxy)butanoic acid (150 mg, 447.39 umol, 1 eq) and (1R,2S,5S)—N-[(1S)-1-cyano-2-[(6R)-5-oxo-4-azaspiro[2.4]heptan-6-yl]ethyl]-6,6-dimethyl-3-azabicyclo [3.1.0]hexane-2-carboxamide (220 mg, 695.33 umol, 1.55 eq) in ACN (10 mL) was cooled to 0° C., then 1-methylimidazole (146.92 mg, 1.79 mmol, 142.64 uL, 4 eq), and TCFH (150.64 mg, 536.87 umol, 1.2 eq) was added at 0° C., the mixture was stirred at 0° C. for 2 h. Upon completion, the reaction mixture was diluted with H₂O (100 Ml) and extracted with DCM (50 mL*3). The combined organic layers were dried over Na₂SO₄, filtered and concentrated under reduced pressure to give a residue. The residue was purified by prep-TLC (SiO₂, Petroleum ether:Ethyl acetate=0:1) to give benzyl N-[(1S,2R)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2-(2,2,2-trifluoroethoxy)propyl]carbamate (150 mg, 236.72 umol, 52.91% yield) as a white solid. MS (ESI) m/z 634.4 [M+H]⁺.

Step 3: (1R,2S,5S)-3-[(2S,3R)-2-amino-3-(2,2,2-trifluoroethoxy)butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide

A solution of benzyl N-[(1S,2R)-1-[(1R,2S,5S)-2-[[(1S)-1-cyano-2-[(3R)-5,5-dimethyl-2-oxo-pyrrolidin-3-yl]ethyl]carbamoyl]-6,6-dimethyl-3-azabicyclo[3.1.0]hexane-3-carbonyl]-2-(2,2,2-trifluoroethoxy)propyl]carbamate (240 mg, 377.55 umol, 1 eq) in isopropanol (10 mL) was added Pd(OH)₂ (240.00 mg, 341.78 umol, 20% purity, 9.05e-1 eq) under H₂ (15 Psi), the mixture was stirred at 25° C. for 15 min. Upon completion, the reaction mixture was filtered and the filtrate was concentrated under reduced pressure to give (1R,2S,5S)-3-[(2S,3R)-2-amino-3-(2,2,2-trifluoroethoxy)butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (100 mg, crude) as a white oil. MS (ESI) m/z 500.4 [M+H]⁺.

Step 4: (1R,2S,5S)—N-[(1S)-1-cyano-2-[(6R)-5-oxo-4-azaspiro[2.4]heptan-6-yl]ethyl]-6,6-dimethyl-3-[(2S,3R)-2-[(2,2,2-trifluoroacetyl)amino]-3-(2,2,2-trifluoroethoxy)butanoyl]-3-azabicyclo[3.1.0]hexane-2-carboxamide

A mixture of (1R,2S,5S)-3-[(2S,3R)-2-amino-3-(2,2,2-trifluoroethoxy)butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (100 mg, 200.19 umol, 1 eq) in MeOH (6 mL) was added Et₃N (60.77 mg, 600.57 umol, 83.59 uL, 3 eq) and methyl 2,2,2-trifluoroacetate (2.54 g, 19.84 mmol, 2.00 mL, 99.09 eq). The mixture was stirred at 20° C. for 16 h. Upon completion, the reaction mixture was concentrated under reduced pressure to give a residue. The residue was purified by prep-HPLC (column: Waters Xbridge Prep OBD C18 150*40 mm*10 um; mobile phase: [water (NH₄HCO₃)-ACN]; B %: 25%-55%, 8 min) to give (1R,2S,5S)—N-[(1S)-1-cyano-2-[(6R)-5-oxo-4-azaspiro[2.4]heptan-6-yl]ethyl]-6,6-dimethyl-3-[(2S,3R)-2-[(2,2,2-trifluoroacetyl)amino]-3-(2,2,2-trifluoroethoxy)butanoyl]-3-azabicyclo[3.1.0]hexane-2-carboxamide (63 mg, 105.79 umol, 52.84% yield, 100% purity) as a white solid. MS (ESI) m/z 596.3 [M+H]⁺.

¹H NMR (400 MHz, DMSO-d₆) δ=9.05 (brd, J=8.3 Hz, 1H), 7.77-7.39 (m, 2H), 5.03-4.86 (m, 1H), 4.47 (d, J=9.1 Hz, 1H), 4.21-4.08 (m, 2H), 4.08-3.99 (m, 1H), 3.99-3.85 (m, 2H), 3.79 (br d, J=10.6 Hz, 1H), 2.71-2.59 (m, 1H), 2.23-2.10 (m, 1H), 2.05-1.89 (m, 2H), 1.87-1.74 (m, 1H), 1.62-1.52 (m, 1H), 1.34 (d, J=7.6 Hz, 1H), 1.21 (d, J=6.3 Hz, 3H), 1.03 (s, 3H), 0.86 (s, 3H), 0.79-0.67 (m, 1H), 0.67-0.47 (m, 3H).

Example 57: Synthesis of (1R,2S,5S)—N-[(1S)-1-cyano-2-[(3R)-5,5-dimethyl-2-oxo-pyrrolidin-3-yl]ethyl]-3-[(2S,3R)-3-(1-cyclopropylcyclopropoxy)-2-[(2,2,2-trifluoroacetyl)amino]butanoyl]-6,6-dimethyl-3-azabicyclo[3.1.0]hexane-2-carboxamide (Compound 324)

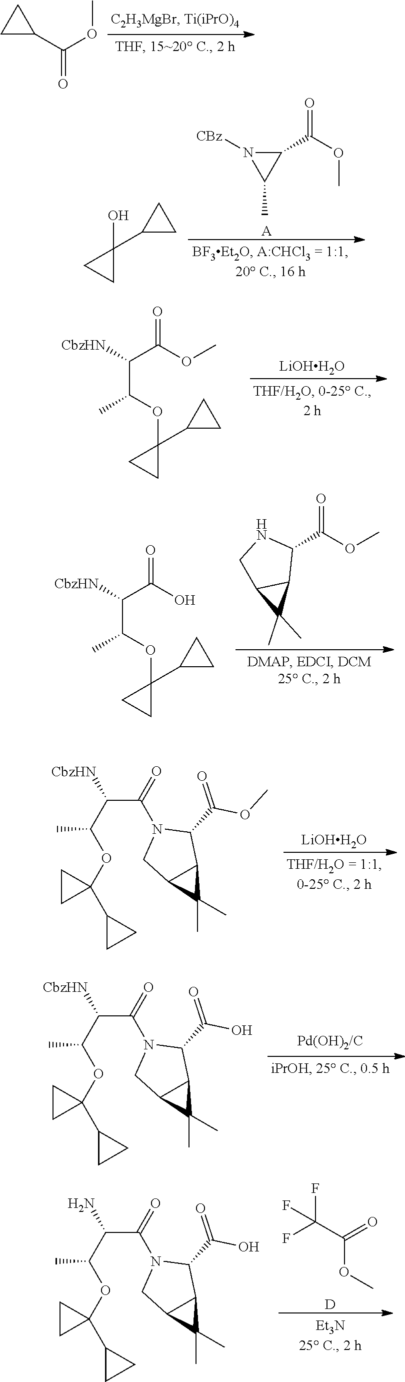

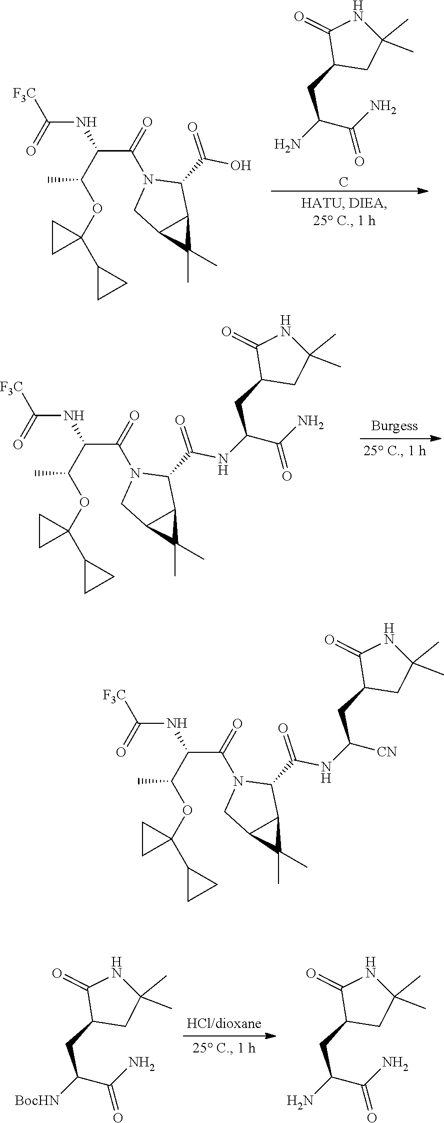

Step 1: (2S)-2-amino-3-[(3R)-5,5-dimethyl-2-oxo pyrrolidin-3-yl]propanamide

A solution of tert-butyl N-[(1S)-2-amino-1-[[(3R)-5,5-dimethyl-2-oxo-pyrrolidin-3-yl]methyl]-2-oxo-ethyl]carbamate (1 g, 3.34 mmol, 1 eq) in HCl/dioxane (4 M, 10 mL, 11.97 eq) was stirred at 25° C. for 1 h. Upon completion, the reaction mixture was concentrated under reduced pressure to give a solid. The residue was dissolved with DCM (10 mL) and concentrated under reduced pressure for three times to give (2S)-2-amino-3-[(3R)-5,5-dimethyl-2-oxo-pyrrolidin-3-yl]propanamide (1.02 g, crude, HCl) as a white solid. MS (ESI) m/z 200.2 [M+H]⁺

Step 2: 1-cyclopropylcyclopropanol

A mixture of methyl cyclopropanecarboxylate (20 g, 199.77 mmol, 20.10 mL, 1 eq) in THE (300 mL) and Ti(i-PrO)₄ (14.19 g, 49.94 mmol, 14.74 mL, 0.25 eq) was cooled to 18° C. EtMgBr (3 M, 139.84 mL, 2.1 eq) was added dropwise over 2 h to control temperature between 15° C. to 20° C. Upon completion, the mixture was stirred for another 2 h then cooled to 5° C. and then quenched with aq. NaHSO₄ (200 mL 20%) while keeping temperature below 10° C. The organic phase was isolated and aqueous phase was re-extracted with petroleum ether (200 mL). The aqueous phase was discarded. The organic phase was combined, washed with sat. aq. NaHCO₃(200 mL), dried over Na₂SO₄ and concentrated to give 1-cyclopropylcyclopropanol (18 g, 165.07 mmol, 82.63% yield, 90% purity) as yellow oil.

Step 3: methyl (3R)-2-(benzyloxycarbonylamino)-3-(1-cyclopropylcyclopropoxy)butanoate

To a solution of 1-cyclopropylcyclopropanol (3 g, 30.57 mmol, 1.1 eq) in CH₃Cl (90 mL) was added O1-benzyl O2-methyl (2S,3S)-3-methylaziridine-1,2-dicarboxylate (6.93 g, 27.79 mmol, 1 eq) and BF₃.Et₂O (839.15 mg, 2.78 mmol, 729.70 uL, 47% purity, 0.1 eq). The mixture was stirred at 25° C. for 16 h. Upon completion, the combined was diluted with water (40 mL) and extracted with DCM (30 mL). And then the solution was extracted with DCM (30 mL*2). The combined organic layers were dried over Na₂SO₄, filtered and concentrated under reduced pressure to give a residue. The residue was purified by column chromatography (SiO₂, Petroleum ether/Ethyl acetate=50/1 to 10/1) to give methyl (3R)-2-(benzyloxycarbonylamino)-3-(1-cyclopropylcyclopropoxy)butanoate (4.5 g, 6.48 mmol, 23.31% yield, 50% purity) as a colourless oil. MS (ESI) m/z 348.3 [M+H]⁺.

Step 4: (3R)-2-(benzyloxycarbonylamino)-3-(1-cyclopropylcyclopropoxy)butanoic acid

To a solution of methyl (3R)-2-(benzyloxycarbonylamino)-3-(1-cyclopropylcyclopropoxy)butanoate (4.5 g, 12.95 mmol, 1 eq) in THE (45 mL) was added LiOH.H₂O (1.90 g, 45.34 mmol, 3.5 eq) in H₂O (45 mL) at 0° C. The mixture was stirred at 25° C. for 2 h. Upon completion, the mixture was extracted with DCM (70 mL). And then the aqueous phase was adjust pH=3 with 1 mol/L HCl, and was extracted with DCM (70 mL*2), dried with Na₂SO₄, filtered and concentrated in vacuum to give (3R)-2-(benzyloxycarbonylamino)-3-(1-cyclopropylcyclopropoxy)butanoic acid (2.02 g, crude) as a yellow oil. MS (ESI) m/z 334.2 [M+H]⁺

Step 5: methyl (1R,2S,5S)-3-[(2S,3R)-2-(benzyloxycarbonylamino)-3-(1-cyclopropylcyclopropoxy)butanoyl]-6,6-dimethyl-3-azabicyclo[3.1.0]hexane-2-carboxylate

To a solution of (3R)-2-(benzyloxycarbonylamino)-3-(1-cyclopropylcyclopropoxy)butanoic acid (2.02 g, 6.06 mmol, 1 eq) in DCM (20 mL) was added DMAP (2.22 g, 18.18 mmol, 3 eq) and methyl (1R,2S,5S)-6,6-dimethyl-3-azabicyclo[3.1.0]hexane-2-carboxylate (1.03 g, 6.06 mmol, 1 eq). To the resulting mixture, EDCI (2.32 g, 12.12 mmol, 2 eq) was added. The mixture was stirred at 25° C. for 2 h. Upon completion, the combined was diluted with water (40 mL) and extracted with DCM (30 mL). And then filtered to give a solution was extracted with DCM (30 mL*2). The combined organic layers were dried over Na₂SO₄, filtered and concentrated under reduced pressure to give a residue. The residue was purified by column chromatography (SiO₂, Petroleum ether/Ethyl acetate=5011 to 8/1) to give methyl (1R,2S,5S)-3-[(2S,3R)-2-(benzyloxycarbonylamino)-3-(1-cyclopropylcyclopropoxy)butanoyl]-6,6-dimethyl-3-azabicyclo[3.1.0]hexane-2-carboxylate (1.68 g, 2.84 mmol, 46.92% yield, 82% purity) as a yellow oil. MS (ESI) m/z 485.4 [M+H]⁺

Step 6: (1R,2S,5S)-3-[(2S,3R)-2-(benzyloxycarbonylamino)-3-(1-cyclopropylcyclopropoxy)butanoyl]-6,6-dimethyl-3-azabicyclo[3.1.0]hexane-2-carboxylic acid

To a solution of methyl (1R,2S,5S)-3-[(2S,3R)-2-(benzyloxycarbonylamino)-3-(1-cyclopropylcyclopropoxy)butanoyl]-6,6-dimethyl-3-azabicyclo[3.1.0]hexane-2-carboxylate (1 g, 2.06 mmol, 1 eq) in THE (15 mL) was added LiOH.H₂O (259.79 mg, 6.19 mmol, 3 eq) in H₂O (15 mL) at 0° C. The mixture was stirred at 25° C. for 2 h. Upon completion, and then the aqueous phase was adjust pH=3 with 1 mol/L HCl, and was extracted with DCM (70 mL*2), dried with Na₂SO₄, filtered and concentrated in vacuum to give (1R,2S,5S)-3-[(2S,3R)-2-(benzyloxycarbonylamino)-3-(1-cyclopropylcyclopropoxy)butanoyl]-6,6-dimethyl-3-azabicyclo[3.1.0]hexane-2-carboxylic acid (900 mg, crude) as a yellow oil. MS (ESI) m/z 471.3 [M+H]⁺.

Step 7: (1R,2S,5S)-3-[(2S,3R)-2-amino-3-(1-cyclopropylcyclopropoxy)butanoyl]-6,6-dimethyl-3-azabicyclo[3.1.0]hexane-2-carboxylic acid

To a solution of (1R,2S,5S)-3-[(2S,3R)-2-(benzyloxycarbonylamino)-3-(1-cyclopropylcyclopropoxy)butanoyl]-6,6-dimethyl-3-azabicyclo[3.1.0]hexane-2-carboxylic acid (900 mg, 1.91 mmol, 1 eq) in i-PrOH (20 mL) was added Pd(OH)₂ (1.34 g, 1.91 mmol, 20% purity, 1 eq) and under N₂ atmosphere. The suspension was degassed and purged with H₂ (3.86 mg, 1.91 mmol, 1 eq) for 3 times. The mixture was stirred under H₂ (15 Psi) at 25° C. for 0.5 h. Upon completion, the reaction mixture was filtered and concentrated under reduced pressure to give (1R,2S,5S)-3-[(2S,3R)-2-amino-3-(1-cyclopropylcyclopropoxy)butanoyl]-6,6-dimethyl-3-azabicyclo[3.1.0]hexane-2-carboxylic acid (470 mg, crude) as a yellow oil. MS (ESI) m/z 337.3 [M+H]⁺

Step 8: (1R,2S,5S)-3-[(2S,3R)-3-(1-cyclopropylcyclopropoxy)-2-[(2,2,2-trifluoroacetyl)amino]butanoyl]-6,6-dimethyl-3-azabicyclo[3.1.0]hexane-2-carboxylic acid

To a solution of (1R,2S,5S)-3-[(2S,3R)-2-amino-3-(1-cyclopropylcyclopropoxy)butanoyl]-6,6-dimethyl-3-azabicyclo[3.1.0]hexane-2-carboxylic acid (470 mg, 1.40 mmol, 1 eq) in methyl 2,2,2-trifluoroacetate (119.38 g, 932.29 mmol, 94.00 mL, 667.33 eq) was added Et₃N (424.10 mg, 4.19 mmol, 583.35 uL, 3 eq). The mixture was stirred at 25° C. for 2 h. Upon completion, the reaction mixture was concentrated under reduced pressure to give (1R,2S,5S)-3-[(2S,3R)-3-(1-cyclopropylcyclopropoxy)-2-[(2,2,2-trifluoroacetyl)amino]butanoyl]-6,6-dimethyl-3-azabicyclo[3.1.0]hexane-2-carboxylic acid (750 mg, crude) as a yellow oil. MS (ESI) m/z 433.2 [M+H]+, and then the residue (60 mg) was purified by prep-HPLC (column: Phenomenex Luna 80*30 mm*3 um; mobile phase: [water(HCl)-ACN]; B %: 40%-60%, 8 min) to give (1R,2S,5S)-3-[(2S,3R)-3-(1-cyclopropylcyclopropoxy)-2-[(2,2,2-trifluoroacetyl)amino]butanoyl]-6,6-dimethyl-3-azabicyclo[3.1.0]hexane-2-carboxylic acid (10 mg, 22.89 umol, 16.50% yield, 99% purity) as a yellow solid. MS (ESI) m/z 433.2 [M+H]⁺.

¹H NMR (400 MHz, DMSO-d₆) δ=13.27-12.60 (m, 1H), 9.91-9.22 (m, 1H), 4.69-4.09 (m, 2H), 4.07-3.42 (m, 3H), 1.58-1.50 (m, 1H), 1.47-1.37 (m, 2H), 1.27 (d, J=6.1 Hz, 2H), 1.20-1.12 (m, 1H), 1.05-0.79 (m, 6H), 0.73-0.62 (m, 1H), 0.54-0.39 (m, 3H), 0.36-0.29 (m, 1H), 0.25-0.17 (m, 1H), 0.12--0.02 (m, 2H)

Step 9: (1R,2S,5S)—N-[(1S)-2-amino-1-[[(3R)-5,5-dimethyl-2-oxo pyrrolidin-3-yl]methyl]-2-oxo-ethyl]-3-[(2S,3R)-3-(1-cyclopropylcyclopropoxy)-2-[(2,2,2-trifluoroacetyl)amino]butanoyl]-6,6-dimethyl-3-azabicyclo[3.1.0]hexane-2-carboxamide

To a solution of (1R,2S,5S)-3-[(2S,3R)-3-(1-cyclopropylcyclopropoxy)-2-[(2,2,2-trifluoroacetyl)amino]butanoyl]-6,6-dimethyl-3-azabicyclo[3.1.0]hexane-2-carboxylic acid (640 mg, 1.48 mmol, 1 eq) in DMF (10 mL) was added (2S)-2-amino-3-[(3R)-5,5-dimethyl-2-oxo-pyrrolidin-3-yl]propanamide (589.78 mg, 2.50 mmol, 1.69 eq, HCl) and DIEA (478.20 mg, 3.70 mmol, 644.47 uL, 2.5 eq), and then HATU (844.11 mg, 2.22 mmol, 1.5 eq) was added. The mixture was stirred at 25° C. for 1 h. Upon completion, the combined was diluted with water (15 mL) and extracted with DCM (10 mL). And then the solution was extracted with DCM (10 mL*2). The combined organic layers were dried over Na₂SO₄, filtered and concentrated under reduced pressure to give a residue. The residue was purified by column chromatography (SiO₂, Petroleum ether/Ethyl acetate=3/1 to Ethyl acetate: Methanol=20/1) to give (1R,2S,5S)—N-[(1S)-2-amino-1-[[(3R)-5,5-dimethyl-2-oxo-pyrrolidin-3-yl]methyl]-2-oxo-ethyl]-3-[(2S,3R)-3-(1-cyclopropylcyclopropoxy)-2-[(2,2,2-trifluoroacetyl)amino]butanoyl]-6,6-dimethyl-3-azabicyclo[3.1.0]hexane-2-carboxamide (800 mg, 1.30 mmol, 88.08% yield) as a yellow oil. MS (ESI) m/z 614.5 [M+H]⁺

Step 10: (1R,2S,5S)—N-[(1S)-1-cyano-2-[(3R)-5,5-dimethyl-2-oxo pyrrolidin-3-yl]ethyl]-3-[(2S,3R)-3-(1-cyclopropylcyclopropoxy)-2-[(2,2,2-trifluoroacetyl)amino]butanoyl]-6,6-dimethyl-3-azabicyclo[3.1.0]hexane-2-carboxamide

To a solution of (1R,2S,5S)—N-[(1S)-2-amino-1-[[(3R)-5,5-dimethyl-2-oxo-pyrrolidin-3-yl]methyl]-2-oxo-ethyl]-3-[(2S,3R)-3-(1-cyclopropylcyclopropoxy)-2-[(2,2,2-trifluoroacetyl)amino]butanoyl]-6,6-dimethyl-3-azabicyclo[3.1.0]hexane-2-carboxamide (800 mg, 1.30 mmol, 1 eq) in DCM (15 mL) was added Burgess reagent (931.99 mg, 3.91 mmol, 3 eq). The mixture was stirred at 25° C. for 1 h. Upon completion, the reaction mixture was quenched by addition water 0.5 mL, and blow dry with nitrogen. The residue was purified by prep-HPLC (column: Waters Xbridge Prep OBD C18 150*40 mm*10 um; mobile phase: [water (NH₄HCO₃)-ACN]; B %: 35%-75%, 8 min) to give (1R,2S,5S)—N-[(1S)-1-cyano-2-[(3R)-5,5-dimethyl-2-oxo-pyrrolidin-3-yl]ethyl]-3-[(2S,3R)-3-(1-cyclopropylcyclopropoxy)-2-[(2,2,2-trifluoroacetyl)amino]butanoyl]-6,6-dimethyl-3-azabicyclo[3.1.0]hexane-2-carboxamide (63 mg, 105.77 umol, 8.11% yield) as a white solid. MS (ESI) m/z 596.4 [M+H]⁺.

¹H NMR (400 MHz, DMSO-d₆) δ=9.83 (brd, J=7.5 Hz, 1H), 8.99 (d, J=8.5 Hz, 1H), 7.81 (s, 1H), 4.93 (ddd, J=5.1, 8.7, 10.7 Hz, 1H), 4.44-4.24 (m, 1H), 4.11 (s, 1H), 4.03-3.86 (m, 2H), 3.72-3.56 (m, 1H), 2.63-2.52 (m, 1H), 2.25-2.10 (m, 1H), 2.05-1.94 (m, 1H), 1.73 (ddd, J=5.1, 10.7, 13.5 Hz, 1H), 1.61-1.48 (m, 2H), 1.44-1.31 (m, 2H), 1.28-1.09 (m, 9H), 1.06-0.79 (m, 6H), 0.65 (td, J=5.6, 11.3 Hz, 1H), 0.53-0.37 (m, 3H), 0.36-0.27 (m, 1H), 0.23-0.12 (m, 1H), 0.11-0.07 (m, 2H)

Example 58: Synthesis of (1R,2S,5S)—N-[(1S)-1-cyan-2-[(6R)-5-oxo-4-azaspiro[2.4]heptan-6-yl]ethyl]-3-[(2S,3R)-3-(cyclopropoxy)-2-[(2,2,2-trifluoroacetyl)amino]butanoyl]-6,6-dimethyl-3-azabicyclo[3.1.0]hexane-2-carboxamide (Compound 326)

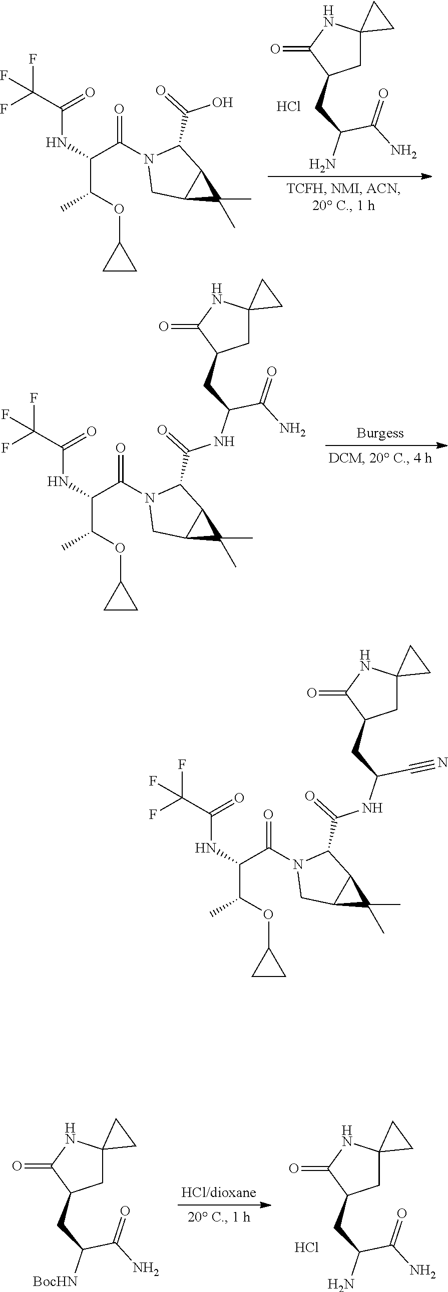

Step 1: (2S)-2-amino-3-[(6R)-5-oxo-4-azaspiro[2.4]heptan-6-yl]propanamide

A solution of tert-butyl N-[(1S)-2-amino-2-oxo-1-[[(6R)-5-oxo-4-azaspiro[2.4]heptan-6-yl]methyl]ethyl]carbamate (400 mg, 1.35 mmol, 1 eq) in HCl/dioxane (4 M, 4 mL, 11.89 eq) was stirred at 20° C. for 1 h. Upon completion, the reaction mixture was concentrated under reduced pressure to give (2S)-2-amino-3-[(6R)-5-oxo-4-azaspiro[2.4]heptan-6-yl]propanamide (300 mg, crude, HCl) as a white solid. MS (ESI) m/z 198.2 [M+H]⁺.

Step 2: (1R,2S,5S)—N-[(1S)-2-amino-2-oxo-1-[[(6R)-5-oxo-4-azaspiro[2.4]heptan-6-yl]methyl]ethyl]-3-[(2S,3R)-3-(cyclopropoxy)-2-[(2,2,2-trifluoroacetyl)amino]butanoyl]-6,6-dimethyl-3-azabicyclo[3.1.0]hexane-2-carboxamide

A solution of (1R,2S,5S)-3-[(2S,3R)-3-(cyclopropoxy)-2-[(2,2,2-trifluoroacetyl)amino]butanoyl]-6,6-dimethyl-3-azabicyclo[3.1.0]hexane-2-carboxylic acid (350 mg, 802.82 umol, 90% purity, 1 eq) in acetonitrile (5 mL) was added (2S)-2-amino-3-[(6R)-5-oxo-4-azaspiro[2.4]heptan-6-yl]propanamide (271.00 mg, 1.04 mmol, 90% purity, 1.3 eq, HCl) and NMI (197.74 mg, 2.41 mmol, 191.98 uL, 3 eq) and then TCFH (450.51 mg, 1.61 mmol, 2 eq). The mixture was stirred at 20° C. for 1 h. Upon completion, the reaction mixture was diluted with H₂O (5 mL) and extracted with ethyl acetate (5 mL*3). The combined organic layers were dried over Na₂SO₄, filtered and concentrated under reduced pressure to give a residue. The residue was purified by column chromatography (SiO₂, DCM:MeOH=10:1) to give (1R,2S,5S)—N-[(1S)-2-amino-2-oxo-1-[[(6R)-5-oxo-4-azaspiro[2.4]heptan-6-yl]methyl]ethyl]-3-[(2S,3R)-3-(cyclopropoxy)-2-[(2,2,2-trifluoroacetyl)amino]butanoyl]-6,6-dimethyl-3-azabicyclo[3.1.0]hexane-2-carboxamide (310 mg, 433.88 umol, 54.04% yield, 80% purity) as a white solid. MS (ESI) m/z 572.4 [M+H]⁺.

Step 3: (1R,2S,5S)—N-[(1S)-1-cyano-2-[(6R)-5-oxo-4-azaspiro[2.4]heptan-6-yl]ethyl]-3-[(2S,3R)-3-(cyclopropoxy)-2-[(2,2,2-trifluoroacetyl)amino]butanoyl]-6,6-dimethyl-3-azabicyclo[3.1.0]hexane-2-carboxamide

A solution of (1R,2S,5S)—N-[(1S)-2-amino-2-oxo-1-[[(6R)-5-oxo-4-azaspiro[2.4]heptan-6-yl]methyl]ethyl]-3-[(2S,3R)-3-(cyclopropoxy)-2-[(2,2,2-trifluoroacetyl)amino]butanoyl]-6,6-dimethyl-3-azabicyclo[3.1.0]hexane-2-carboxamide (300 mg, 367.40 umol, 70% purity, 1 eq) in DCM (3 mL) was added Burgess reagent (262.66 mg, 1.10 mmol, 3 eq). The mixture was stirred at 20° C. for 4 h. Upon completion, the reaction mixture was filtered and concentrated under reduced pressure to give a residue. The residue was purified by prep-HPLC (column: Waters Xbridge Prep OBD C18 150*40 mm*10 um; mobile phase: [water (NH₄HCO₃)-ACN]; B %: 35%-55%, 8 min) to give (1R,2S,5S)—N-[(1S)-1-cyano-2-[(6R)-5-oxo-4-azaspiro[2.4]heptan-6-yl]ethyl]-3-[(2S,3R)-3-(cyclopropoxy)-2-[(2,2,2-trifluoroacetyl)amino]butanoyl]-6,6-dimethyl-3-azabicyclo[3.1.0]hexane-2-carboxamide (65 mg, 117.42 umol, 31.96% yield, 100% purity) as a white solid. MS (ESI) m/z 554.2 [M+H]⁺.

¹H NMR (400 MHz, METHANOL-d₄) δ=5.03 (dd, J=5.5, 10.4 Hz, 1H), 4.52 (d, J=7.6 Hz, 1H), 4.26 (s, 1H), 4.02 (dd, J=5.5, 10.3 Hz, 1H), 3.98-3.84 (m, 2H), 3.46 3.37 (m, 1H), 2.90 (dq, J=5.1, 9.3 Hz, 1H), 2.36 (ddd, J=5.0, 10.5, 13.9 Hz, 1H), 2.18 2.03 (m, 2H), 1.96 (ddd, J=5.7, 9.9, 13.9 Hz, 1H), 1.64 (dd, J=5.5, 7.4 Hz, 1H), 1.42 (d, J=7.5 Hz, 1H), 1.29 (d, J=6.3 Hz, 3H), 1.09 (s, 3H), 0.96 (s, 3H), 0.91-0.84 (m, 1H), 0.78 0.64 (m, 3H), 0.60-0.49 (m, 3H), 0.45-0.38 (m, 1H)

Example 59: Synthesis of (1R,2S,5S)—N-[(1S)-1-cyan-2-[(6R)-5-oxo-4-azaspiro[2.4]heptan-6-yl]ethyl]-3-[(2S,3R)-3-(cyclobutoxy)-2-[(2,2,2-trifluoroacetyl)amino]butanoyl]-6,6-dimethyl-3-azabicyclo[3.1.0]hexane-2-carboxamide (Compound 327)

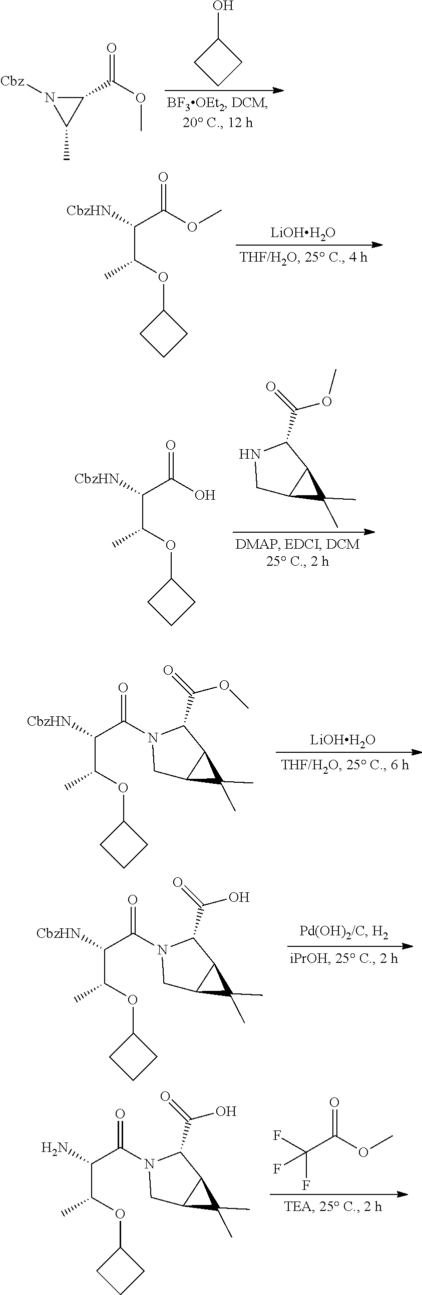

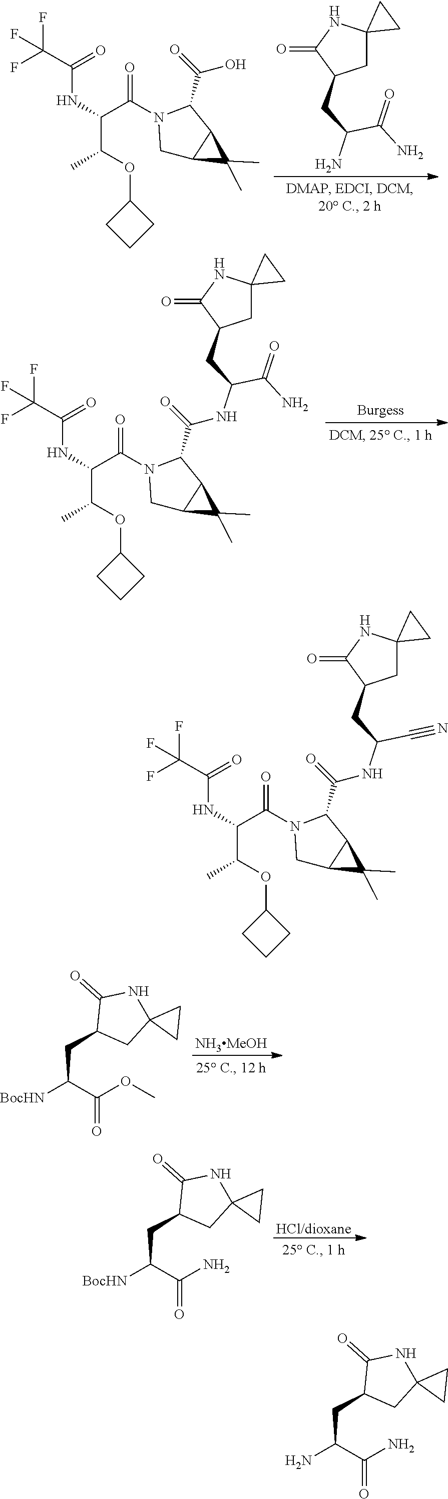

Step 1: tert-butyl N-[(1S)-2-amino-2-oxo-1-[[(6R)-5-oxo-4-azaspiro[2.4]heptan-6-yl]methyl]ethyl]carbamate

A solution of methyl (2S)-2-(tert-butoxycarbonylamino)-3-[(6R)-5-oxo-4-azaspiro[2.4]heptan-6-yl]propanoate (2 g, 6.40 mmol, 1 eq) in NH₃.MeOH (7 M, 20 mL, 21.87 eq) was stirred at 25° C. for 12 h. Upon completion, the residue was concentrated in vacuum, and washed with DCM (20 mL*2) and concentrated, to give tert-butyl N-[(1S)-2-amino-2-oxo-1-[[(6R)-5-oxo-4-azaspiro[2.4]heptan-6-yl]methyl]ethyl]carbamate (1.9 g, crude) as a yellow solid.

Step 2: (2S)-2-amino-3-[(6R)-5-oxo-4-azaspiro[2.4]heptan-6-yl]propanamide

A solution of tert-butyl N-[(1S)-2-amino-2-oxo-1-[[(6R)-5-oxo-4-azaspiro[2.4]heptan-6-yl]methyl]ethyl]carbamate (1.9 g, 6.39 mmol, 1 eq) was added HCl/dioxane (4 M, 10 mL) then the mixture was stirred at 25° C. for 1 h. Upon completion, the residue was concentrated in vacuum, and washed with DCM (40 mL*2) and concentrated to give (2S)-2-amino-3-[(6R)-5-oxo-4-azaspiro[2.4]heptan-6-yl]propanamide (1.2 g, crude) as a yellow solid.

Step 3: methyl (2S,3R)-2-(benzyloxycarbonylamino)-3-(cyclobutoxy)butanoate

To a solution of O1-benzyl O2-methyl (2S,3S)-3-methylaziridine-1,2-dicarboxylate (5 g, 20.06 mmol, 1 eq) and cyclobutanol (1.74 g, 24.07 mmol, 1.2 eq) in CHCl₃ (50 mL) was added BF₃.Et₂O (605.75 mg, 2.01 mmol, 526.74 uL, 47% purity, 0.1 eq), the mixture was stirred at 20° C. for 12 h. Upon completion, the reaction mixture was quenched by addition H₂O 100 mL at 0° C., and then washed with 10% aqueous citric acid solution (2*100 ml) and brine (2*100 ml), and then extracted with DCM (2000 mL), dried over NA₂SO₄, filtered and concentrated under reduced pressure to give a residue. The residue was purified by column chromatography (SiO₂, Petroleum ether/Ethyl acetate=1/Oto 5/1). To give methyl (2S,3R)-2-(benzyloxycarbonylamino)-3-(cyclobutoxy)butanoate (3.66 g, 9.34 mmol, 46.56% yield, 82% purity) as a white oil

Step 4: (2S,3R)-2-(benzyloxycarbonylamino)-3-(cyclobutoxy)butanoic acid

To a solution of methyl (2S,3R)-2-(benzyloxycarbonylamino)-3-(cyclobutoxy)butanoate (3.66 g, 11.39 mmol, 1 eq) and LiOH.H₂O (1.67 g, 39.86 mmol, 3.5 eq) in THE (36 mL) and then H₂O (12 mL) was stirred at 25° C. for 1 h. LCMS showed the reaction was not completed, then the mixture was stirred at 25° C. for 3 h. Upon completion, the residue was concentrated and adjusted to pH-4 with 2 N HCl, and was extracted with ethyl acetate (50 mL*2), the combined organicphase was washed with brine (30 mL), dried over Na₂SO₄ and concentrated to give (2S,3R)-2-(benzyloxycarbonylamino)-3-(cyclobutoxy)butanoic acid (3.5 g, crude) as a white oil.

Step 5: methyl (1R,2S,5S)-3-[(2S,3R)-2-(benzyloxycarbonylamino)-3-(cyclobutoxy)butanoyl]-6,6-dimethyl-3-azabicyclo[3.1.0]hexane-2-carboxylate

To a solution of methyl (1R,2S,5S)-6,6-dimethyl-3-azabicyclo[3.1.0]hexane-2-carboxylate (1.93 g, 11.39 mmol, 1 eq) and (2S,3R)-2-(benzyloxycarbonylamino)-3-(cyclobutoxy)butanoic acid (3.5 g, 11.39 mmol, 1 eq) in DCM (35 mL) was added DMAP (4.17 g, 34.16 mmol, 3 eq) and EDCI (4.37 g, 22.78 mmol, 2 eq). The resulting mixture was stirred at 25° C. for 2 h. Upon completion, the resulting solution was poured into brine (50 ml) and then extracted with EtOAc (50 mL*2). The combined organic phase was washed with 7% citric acid (50 mL*2), NaHCO₃(30 mL), and brine (50 mL*3), dried over Na₂SO₄, filtered and concentrated to give a crude product. The residue was purified by column chromatography (SiO₂, Petroleum ether/Ethyl acetate=I/O to 9/1) to give methyl (1R,2S,5S)-3-[(2S,3R)-2-(benzyloxycarbonylamino)-3-(cyclobutoxy)butanoyl]-6,6-dimethyl-3-azabicyclo[3.1.0]hexane-2-carboxylate (3.66 g, 7.58 mmol, 66.58% yield, 95% purity) as a while oil.

Step 6: (1R,2S,5S)-3-[(2S,3R)-2-(benzyloxycarbonylamino)-3-(cyclobutoxy)butanoyl]-6,6-dimethyl-3-azabicyclo[3.1.0]hexane-2-carboxylic acid

To a solution of methyl (1R,2S,5S)-3-[(2S,3R)-2-(benzyloxycarbonylamino)-3-(cyclobutoxy)butanoyl]-6,6-dimethyl-3-azabicyclo[3.1.0]hexane-2-carboxylate (3.66 g, 7.98 mmol, 1 eq) and LiOH.H₂O (1.17 g, 27.94 mmol, 3.5 eq) in THE (36 mL) was added H₂O (12 mL), and then the resulting mixture was stirred at 25° C. for 6 h. Upon completion, the residue was concentrated and adjusted to pH-4 with 2 N HCl, and was extracted with ethyl acetate (50 mL*2), the combined organicphase was washed with brine (30 mL), dried over Na₂SO₄ and concentrated to give (1R,2S,5S)-3-[(2S,3R)-2-(benzyloxycarbonylamino)-3-(cyclobutoxy)butanoyl]-6,6-dimethyl-3-azabicyclo[3.1.0]hexane-2-carboxylic acid (3.5 g, crude) as a white oil.

Step 7: (1R,2S,5S)-3-[(2S,3R)-2-amino-3-(cyclobutoxy)butanoyl]-6,6-dimethyl-3-azabicyclo[3.1.0]hexane-2-carboxylic acid

To a solution of (1R,2S,5S)-3-[(2S,3R)-2-(benzyloxycarbonylamino)-3-(cyclobutoxy)butanoyl]-6,6-dimethyl-3-azabicyclo[3.1.0]hexane-2-carboxylic acid (3.5 g, 7.87 mmol, 1 eq) in i-PrOH (30 mL) was added Pd(OH)₂ (5.53 g, 7.87 mmol, 20% purity, 1 eq) under N₂ atmosphere. The suspension was degassed and purged with H₂ (15.87 mg, 7.87 mmol, 1 eq) for 3 times. The mixture was stirred under H₂ (15 Psi) at 25° C. for 2 hr, Upon completion, the reaction was filtered and concentrated under reduced pressure to give a residue. To give (1R,2S,5S)-3-[(2S,3R)-2-amino-3-(cyclobutoxy)butanoyl]-6,6-dimethyl-3-azabicyclo[3.1.0]hexane-2-carboxylic acid (1.92 g, crude) as a white solid.

Step 8: (1R,2S,5S)-3-[(2S,3R)-3-(cyclobutoxy)-2-[(2,2,2-trifluoroacetyl)amino]butanoyl]-6,6-dimethyl-3-azabicyclo[3.1.0]hexane-2-carboxylic acid

To a solution of (1R,2S,5S)-3-[(2S,3R)-2-amino-3-(cyclobutoxy)butanoyl]-6,6-dimethyl-3-azabicyclo[3.1.0]hexane-2-carboxylic acid (1.92 g, 6.19 mmol, 1 eq) in methyl 2,2,2-trifluoroacetate (7.92 g, 61.86 mmol, 6.24 mL, 10 eq) was added Et₃N (1.88 g, 18.56 mmol, 2.58 mL, 3 eq), and then the resulting mixture was stirred at 25° C. for 2 h. Upon completion, the reaction was concentrated under reduced pressure to give (1R,2S,5S)-3-[(2S,3R)-3-(cyclobutoxy)-2-[(2,2,2-trifluoroacetyl)amino]butanoyl]-6,6-dimethyl-3-azabicyclo[3.1.0]hexane-2-carboxylic acid (3.4 g, crude) as a yellow oil.

Step 9: (1R,2S,5S)—N-[(1S)-2-amino-2-oxo-1-[[(6R)-5-oxo-4-azaspiro[2.4]heptan-6-yl]methyl]ethyl]-3-[(2S,3R)-3-(cyclobutoxy)-2-[(2,2,2-trifluoroacetyl)amino]butanoyl]-6,6-dimethyl-3-azabicyclo[3.1.0]hexane-2-carboxamide

To a solution of (2S)-2-amino-3-[(6R)-5-oxo-4-azaspiro[2.4]heptan-6-yl]propanamide (509.59 mg, 2.58 mmol, 1.5 eq) and(1R,2S,5S)-3-[(2S,3R)-3-(cyclobutoxy)-2-[(2,2,2-trifluoroacetyl)amino]butanoyl]-6,6-dimethyl-3-azabicyclo[3.1.0]hexane-2-carboxylic acid (1 g, 1.72 mmol, 70% purity, 1 eq) in ACN (10 mL) and then DMAP (631.29 mg, 5.17 mmol, 3 eq) and EDCI (660.40 mg, 3.44 mmol, 2 eq) was added, then the mixture was stirred at 25° C. for 2 h. Upon completion, the reaction mixture was diluted with H₂O 20 mL and then extracted with EA (20 mL*2). The combined organic layers were washed with 15% citric acid (20 mL*2), NaHCO₃(20 mL) and brine (20 mL) dried over Na₂SO₄, filtered and concentrated under reduced pressure to give a residue. The residue was purified by column chromatography (SiO₂, Petroleum ether/Ethyl acetate=1/2 to 0/1) to give (1R,2S,5S)—N-[(1S)-2-amino-2-oxo-1-[[(6R)-5-oxo-4-azaspiro[2.4]heptan-6-yl]methyl]ethyl]-3-[(2S,3R)-3-(cyclobutoxy)-2-[(2,2,2-trifluoroacetyl)amino]butanoyl]-6,6-dimethyl-3-azabicyclo[3.1.0]hexane-2-carboxamide (623 mg, 872.35 umol, 50.65% yield, 82% purity) as a yellow solid.

Step 10: (1R,2S,5S)—N-[(1S)-1-cyano-2-[(6R)-5-oxo-4-azaspiro[2.4]heptan-6-yl]ethyl]-3-[(2S,3R)-3-(cyclobutoxy)-2-[(2,2,2-trifluoroacetyl)amino]butanoyl]-6,6-dimethyl-3-azabicyclo[3.1.0]hexane-2-carboxamide

To a solution of (1R,2S,5S)—N-[(1S)-2-amino-2-oxo-1-[[(6R)-5-oxo-4-azaspiro[2.4]heptan-6-yl]methyl]ethyl]-3-[(2S,3R)-3-(cyclobutoxy)-2-[(2,2,2-trifluoroacetyl)amino]butanoyl]-6,6-dimethyl-3-azabicyclo[3.1.0]hexane-2-carboxamide (623 mg, 1.06 mmol, 1 eq) in DCM (10 mL) was added Burgess reagent (760.57 mg, 3.19 mmol, 3 eq) and then the resulting mixture was stirred at 25° C. for 1 h. Upon completion, the reaction was quenched with water (1 mL), and was blowed to dry with N₂, the residue was purified by prep-HPLC (column: Waters Xbridge Prep OBD C18 150*40 mm*10 um; mobile phase: [water(NH₄HCO₃)-ACN]; B %: 20%-60%, 8 min). (1R,2S,5S)—N-[(1S)-1-cyano-2-[(6R)-5-oxo-4-azaspiro[2.4]heptan-6-yl]ethyl]-3-[(2S,3R)-3-(cyclobutoxy)-2-[(2,2,2-trifluoroacetyl)amino]butanoyl]-6,6-dimethyl-3-azabicyclo[3.1.0]hexane-2-carboxamide (64.58 mg, 113.78 umol, 10.69% yield, 100% purity) was obtained as a white solid. MS (ESI) m/z 568.3 [M+H]⁺.

¹H NMR (400 MHz, DMSO-d₆) δ=10.14-9.71 (m, 1H), 9.18-8.63 (m, 1H), 7.98-7.63 (m, 1H), 5.03-4.87 (m, 1H), 4.50-4.31 (m, 1H), 4.17-4.08 (m, 1H), 4.05-3.89 (m, 2H), 3.81-3.60 (m, 2H), 2.72-2.57 (m, 1H), 2.23-2.03 (m, 3H), 1.96-1.71 (m, 4H), 1.62-1.49 (m, 2H), 1.46-1.19 (m, 3H), 1.10 (d, J=6.4 Hz, 2H), 1.06-0.96 (m, 5H), 0.89 0.80 (m, 3H), 0.79-0.66 (m, 1H), 0.62-0.51 (m, 2H)

Example 60: Synthesis of (1R,2S,5S)—N-[(1S)-1-cyan-2-[(6R)-5-oxo-4-azaspiro[2.4]heptan-6-yl]ethyl]-6,6-dimethyl-3-[2-(1-methykydopropyl)-2-[(2,2,2-trifluoroacetyl)amino]acetyl]-3-azabicyclo[3.1.0]hexane-2-carboxamide (Compound 341 Isomer 1)

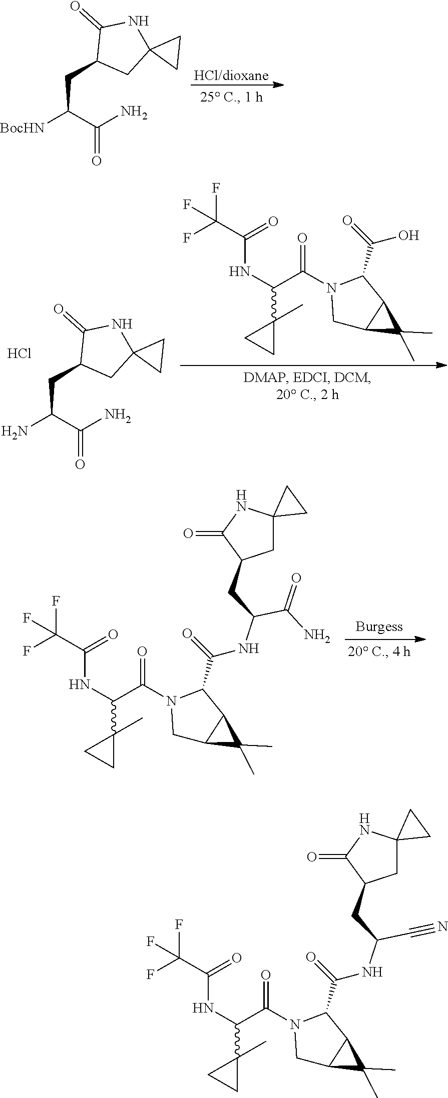

Step 1: (2S)-2-amino-3-[(6R)-5-oxo-4-azaspiro[2.4]heptan-6-yl]propanamide

A mixture of tert-butyl N-[(1S)-2-amino-2-oxo-1-[[(6R)-5-oxo-4-azaspiro[2.4]heptan-6-yl]methyl]ethyl]carbamate (1 g, 2.86 mmol, 85% purity, 1 eq) in HCl/dioxane (4 M, 100 mL, 139.93 eq) was stirred at 25° C. for 1 h. Upon completion, the reaction was concentrated under reduced pressure to give (2S)-2-amino-3-[(6R)-5-oxo-4-azaspiro[2.4]heptan-6-yl]propanamide (800 mg, crude, HCl) as yellow solid. MS (ESI) m/z 198.2 [M+H]⁺.

Step 2: (1R,2S,5S)—N-[(1S)-2-amino-2-oxo-1-[[(6R)-5-oxo-4-azaspiro[2.4]heptan-6-yl]methyl]ethyl]-6,6-dimethyl-3-[(2S)-2-(1-methylcyclopropyl)-2-[(2,2,2-trifluoroacetyl)amino]acetyl]-3-azabicyclo[3.1.0]hexane-2-carboxamide

To a mixture of (2S)-2-amino-3-[(6R)-5-oxo-4-azaspiro[2.4]heptan-6-yl]propanamide (300 mg, 1.37 mmol, 90% purity, 1 eq) in DCM (4 mL) was added (1R,2S,5S)-6,6-dimethyl-3-[2-(1-methylcyclopropyl)-2-[(2,2,2-trifluoroacetyl)amino]acetyl]-3-azabicyclo[3.1.0]hexane-2-carboxylic acid (496.41 mg, 1.37 mmol, 1 eq), DMAP (501.73 mg, 4.11 mmol, 3 eq) and EDCI (524.85 mg, 2.74 mmol, 2 eq), and then the resulting mixture was stirred at 20° C. for 2 h. Upon completion, the reaction mixture was quenched by addition into H₂O (15 mL), and then extracted with DCM (10 mL*2). The combined organic layers were washed with HCl (1M, 20 mL), then washed with brine (30 mL), dried over Na₂SO₄, filtered and concentrated under reduced pressure. The residue was purified by prep-TLC (SiO₂, EA, Rf=0.65, UV 254) to give (1R,2S,5S)—N-[(1S)-2-amino-2-oxo-1-[[(6R)-5-oxo-4-azaspiro[2.4]heptan-6-yl]methyl]ethyl]-6,6-dimethyl-3-[(2S)-2-(1-methylcyclopropyl)-2-[(2,2,2-trifluoroacetyl)amino]acetyl]-3-azabicyclo[3.1.0]hexane-2-carboxamide (200 mg, 344.93 umol, 25.18% yield, 93.4% purity) as yellow solid. MS (ESI) m/z 542.4 [M+H]⁺.

Step 3: (1R,2S,5S)—N-[(1S)-1-cyano-2-[(6R)-5-oxo-4-azaspiro[2.4]heptan-6-yl]ethyl]-6,6-dimethyl-3-[2-(1-methylcyclopropyl)-2-[(2,2,2-trifluoroacetyl)amino]acetyl]-3-azabicyclo[3.1.0]hexane-2-carboxamide Isomer 1

To a mixture of (1R,2S,5S)—N-[(1S)-2-amino-2-oxo-1-[[(6R)-5-oxo-4-azaspiro[2.4]heptan-6-yl]methyl]ethyl]-6,6-dimethyl-3-[2-(1-methylcyclopropyl)-2-[(2,2,2-trifluoroacetyl)amino]acetyl]-3-azabicyclo[3.1.0]hexane-2-carboxamide (200 mg, 369.30 umol, 1 eq) in DCM (6 mL) was added burgess reagent (264.02 mg, 1.11 mmol, 3 eq), and the resulting mixture was stirred at 20° C. for 4 h. Upon completion, the reaction mixture was quenched by addition H₂O (2 mL), and then concentrated under reduced pressure to give a residue. The residue was purified by prep-HPLC (column: Waters Xbridge Prep OBD C18 150*40 mm*10 um; mobile phase: [water (NH₄HCO₃)-ACN]; B %: 35%-55%, 8 min) to give (1R,2S,5S)—N-[(1S)-1-cyano-2-[(6R)-5-oxo-4-azaspiro[2.4]heptan-6-yl]ethyl]-6,6-dimethyl-3-[2-(1-methylcyclopropyl)-2-[(2,2,2-trifluoroacetyl)amino]acetyl]-3-azabicyclo[3.1.0]hexane-2-carboxamide Isomer 1 (81.5 mg, 150.56 umol, 40.77% yield, 96.72% purity) as a white solid. MS (ESI) m/z 524.3 [M+H]⁺.

¹H NMR (400 MHz, DMSO-d₆) δ=9.67 (br d, J=6.2 Hz, 1H), 9.05 (d, J=8.5 Hz, 1H), 7.75 (s, 1H), 4.98-4.87 (m, 1H), 4.38 (d, J=6.4 Hz, 1H), 4.12 (s, 1H), 3.85-3.75 (m, 1H), 3.69 (br d, J=10.5 Hz, 1H), 2.66-2.54 (m, 1H), 2.17 (ddd, J=4.5, 10.6, 13.5 Hz, 1H), 2.03-1.90 (m, 2H), 1.80 (ddd, J=5.5, 10.8, 13.3 Hz, 1H), 1.65-1.57 (m, 1H), 1.29 (d, J=7.6 Hz, 1H), 1.07 (s, 3H), 1.04 (s, 3H), 0.93 (s, 3H), 0.81-0.69 (m, 3H), 0.63-0.51 (m, 3H), 0.34-0.23 (m, 2H).

Example 61: Synthesis of (1R,2S,5S)—N-[(1S)-1-cyan-2-[(6R)-5-oxo-4-azaspiro[2.4]heptan-6-yl]ethyl]-6,6-dimethyl-3-[(2S)-2-(1-methykydopropyl)-2-[(2,2,2-trifluoroacetyl)amino]acetyl]-3-azabicyclo[3.1.0]hexane-2-carboxamide (Compound 341 Isomer 2)

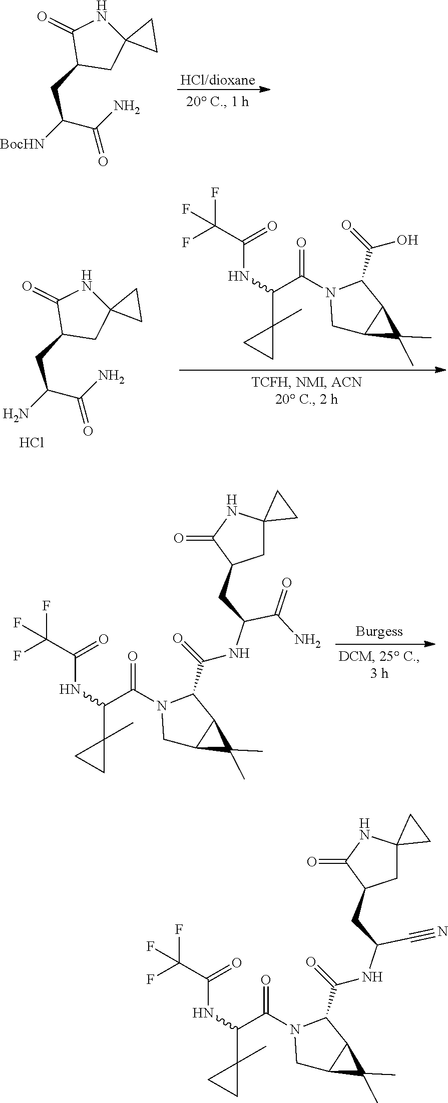

Step 1: (2S)-2-amino-3-[(6R)-5-oxo-4-azaspiro[2.4]heptan-6-yl]propanamide

A solution of tert-butyl N-[(1S)-2-amino-2-oxo-1-[[(6R)-5-oxo-4-azaspiro[2.4]heptan-6-yl]methyl]ethyl]carbamate (500 mg, 1.68 mmol, 1 eq) in HCl/dioxane (4 M) was stirred at 20° C. for 1 h. Upon completion, the reaction mixture was concentrated under reduced pressure to get (2S)-2-amino-3-[(6R)-5-oxo-4-azaspiro[2.4]heptan-6-yl]propanamide (260 mg, crude, HCl) as white solid.

Step 2: (1R,2S,5S)—N-[(1S)-2-amino-2-oxo-1-[[(6R)-5-oxo-4-azaspiro[2.4]heptan-6-yl]methyl]ethyl]-6,6-dimethyl-3-[(2S)-2-(1-methylcyclopropyl)-2-[(2,2,2-trifluoroacetyl)amino]acetyl]-3-azabicyclo[3.1.0]hexane-2-carboxamide

To a solution of (1R,2S,5S)-6,6-dimethyl-3-[2-(1-methylcyclopropyl)-2-[(2,2,2-trifluoroacetyl)amino]acetyl]-3-azabicyclo[3.1.0]hexane-2-carboxylic acid (403.13 mg, 1.11 mmol, 1 eq) in ACN (10 mL) was added (2S)-2-amino-3-[(6R)-5-oxo-4-azaspiro[2.4]heptan-6-yl]propanamide (260 mg, 1.11 mmol, 1 eq, HCl), TCFH (468.24 mg, 1.67 mmol, 1.5 eq) and NMI (274.02 mg, 3.34 mmol, 266.04 uL, 3 eq). The mixture was stirred at 20° C. for 2 h.

Upon completion, the reaction mixture was quenched by addition H₂O (0.5 mL) at 25° C., and then diluted with H₂O (10 mL) and extracted with ethyl acetate (5 mL*2). The combined organic layers were washed with sat. NaCl (5 mL), dried over Na₂SO₄, filtered and concentrated under reduced pressure to give a residue. The residue was purified by column chromatography (SiO₂, dichloromethane:methyl alcohol=1:0 to 50:1) to get (1R,2S,5S)—N-[(1S)-2-amino-2-oxo-1-[[(6R)-5-oxo-4-azaspiro[2.4]heptan-6-yl]methyl]ethyl]-6,6-dimethyl-3-[(2S)-2-(1-methylcyclopropyl)-2-[(2,2,2-trifluoroacetyl)amino]acetyl]-3-azabicyclo[3.1.0]hexane-2-carboxamide (300 mg, 227.12 umol, 20.41% yield, 41% purity) as white solid. MS (ESI) m/z 542.4 [M+H]⁺

Step 3: (1R,2S,5S)—N-[(1S)-1-cyano-2-[(6R)-5-oxo-4-azaspiro[2.4]heptan-6-yl]ethyl]-6,6-dimethyl-3-[(2S)-2-(1-methylcyclopropyl)-2-[(2,2,2-trifluoroacetyl)amino]acetyl]-3-azabicyclo[3.1.0]hexane-2-carboxamide

To a solution of (1R,2S,5S)—N-[(1S)-2-amino-2-oxo-1-[[(6R)-5-oxo-4-azaspiro[2.4]heptan-6-yl]methyl]ethyl]-6,6-dimethyl-3-[(2S)-2-(1-methylcyclopropyl)-2-[(2,2,2-trifluoroacetyl)amino]acetyl]-3-azabicyclo[3.1.0]hexane-2-carboxamide (300 mg, 553.95 umol, 1 eq) in DCM (10 mL) was added burgess reagent (396.03 mg, 1.66 mmol, 3 eq). The mixture was stirred at 25° C. for 3 h. Upon completion, the residue was purified by prep-HPLC (column: Waters Xbridge Prep OBD C18 150*40 mm*10 um; mobile phase: [water(NH₄HCO₃)-ACN]; B %: 30%-60%, 8 min) to give desired compound as a white solid, which was further purified by SFC (column: DAICEL CHIRALPAK IC(250 mm*30 mm,10 um); mobile phase: [Neu-IPA]; B %: 26%-26%, 8 min) to get (1R,2S,5S)—N-[(1S)-1-cyano-2-[(6R)-5-oxo-4-azaspiro[2.4]heptan-6-yl]ethyl]-6,6-dimethyl-3-[(2S)-2-(1-methylcyclopropyl)-2-[(2,2,2-trifluoroacetyl)amino]acetyl]-3-azabicyclo[3.1.0]hexane-2-carboxamide (69 mg, 131.79 umol, 23.79% yield, 100% purity) as white solid. MS (ESI) m/z 524.2 [M+H]⁺

¹H NMR (400 MHz, DMSO-d₆) δ=9.94-9.61 (m, 1H), 9.26-8.69 (m, 1H), 8.00-7.76 (m, 1H), 4.93 (q, J=7.9 Hz, 1H), 4.39-4.25 (m, 1H), 4.09 (s, 1H), 3.87 (br dd, J =5.5, 10.2 Hz, 1H), 3.67-3.52 (m, 1H), 2.71 (m, 1H), 2.22-1.73 (m, 4H), 1.62-1.41 (m, 1H), 1.38-1.29 (m, 1H), 1.07-0.95 (m, 8H), 0.85-0.69 (m, 3H), 0.66-0.09 (m, 6H)

Example 62: Synthesis of (1R,2S,5S)—N-[(1S)-1-cyano-2-[(3R)-5,5-dimethyl-2-oxo-pyrrolidin-3-yl]ethyl]-6,6-dimethyl-3-[2-(1-methykydopropyl)-2-[(2,2,2-trifluoroacetyl)amino]acetyl]-3-azabicyclo[3.1.0]hexane-2-carboxamide (Compound 340 Isomer 1)

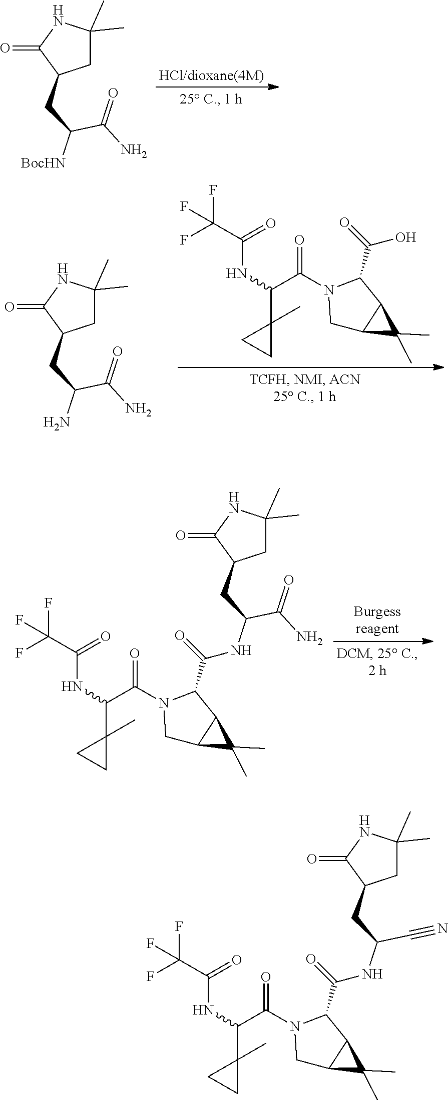 [SMILES: CC1(C)C[C@@H](C[C@H](NC(=O)OC(C)(C)C)C(N)=O)C(=O)N1 ; "HCl/dioxane(4M)" ; CC1(C)C[C@@H](C[C@H](N)C(N)=O)C(=O)N1 ; CC1(C(NC(=O)C(F)(F)F)C(=O)N2C[C@H]3[C@@H]([C@H]2C(=O)O)C3(C)C)CC1 ; CC1(C)C[C@@H](C[C@H](NC(=O)[C@@H]2[C@@H]3[C@H](CN2C(=O)C(NC(=O)C(F)(F)F)C2(C)CC2)C3(C)C)C(N)=O)C(=O)N1 ; CC1(C)C[C@@H](C[C@@H](C#N)NC(=O)[C@@H]2[C@@H]3[C@H](CN2C(=O)C(NC(=O)C(F)(F)F)C2(C)CC2)C3(C)C)C(=O)N1]

Step 1: (2S)-2-amino-3-[(3R)-5,5-dimethyl-2-oxo pyrrolidin-3-yl]propanamide

A solution of tert-butyl N-[(1S)-2-amino-1-[[(3R)-5,5-dimethyl-2-oxo-pyrrolidin-3-yl]methyl]-2-oxo-ethyl]carbamate (500 mg, 1.67 mmol, 1 eq) in HCl/dioxane (4 M, 20.00 mL, 47.90 eq) was stirred at 25° C. for 1 h. Upon completion, the reaction mixture was concentrated under reduced pressure to give (2S)-2-amino-3-[(3R)-5,5-dimethyl-2-oxo-pyrrolidin-3-yl]propanamide (312.25 mg, crude, HCl) as a white solid. MS (ESI) m/z 200.1 [M+H]⁺.

Step 2: (1R,2S,5S)—N-[(1S)-2-amino-1-[[(3R)-5,5-dimethyl-2-oxo pyrrolidin-3-yl]methyl]-2-oxo-ethyl]-6,6-dimethyl-3-[2-(1-methylcyclopropyl)-2-[(2,2,2-trifluoroacetyl)amino]acetyl]-3-azabicyclo[3.1.0]hexane-2-carboxamide

A solution of (1R,2S,5S)-6,6-dimethyl-3-[2-(1-methylcyclopropyl)-2-[(2,2,2-trifluoroacetyl)amino] acetyl]-3-azabicyclo[3.1.0]hexane-2-carboxylic acid (400 mg, 1.10 mmol, 1 eq) in ACN (10 mL) was added NMI (271.91 mg, 3.31 mmol, 263.99 uL, 3 eq) and TCFH (619.48 mg, 2.21 mmol, 2 eq). And then (2S)-2-amino-3-[(3R)-5,5-dimethyl-2-oxo-pyrrolidin-3-yl]propanamide (312.25 mg, 1.32 mmol, 1.2 eq, HCl) was added. The mixture was stirred at 25° C. for 1 h. Upon completion, the reaction mixture was diluted with H₂O (20 mL) and extracted with Ethyl acetate 45 mL (15 mL*3). The combined organic layers were washed with brine 100 mL (20 mL*5), dried over Na₂SO₄, filtered and concentrated under reduced pressure to give a residue. The residue was purified by column chromatography (SiO₂, Petroleum ether:Ethyl acetate=5:1 to Ethyl acetate:MeOH=10:1) was concentrated under reduced pressure to give (1R,2S,5S)—N-[(1S)-2-amino-1-[[(3R)-5,5-dimethyl-2-oxo-pyrrolidin-3-yl]methyl]-2-oxo-ethyl]-6,6-dimethyl-3-[2-(1-methylcyclopropyl)-2-[(2,2,2-trifluoroacetyl)amino]acetyl]-3-azabicyclo[3.1.0]hexane-2-carboxamide (440 mg, 489.72 umol, 44.36% yield, 60.5% purity) as a light yellow oil. MS (ESI) m/z 544.3 [M+H]⁺.

Step 3: (1R,2S,5S)—N-[(1S)-1-cyano-2-[(3R)-5,5-dimethyl-2-oxo pyrrolidin-3-yl]ethyl]-6,6-dimethyl-3-[2-(1-methylcyclopropyl)-2-[(2,2,2-trifluoroacetyl)amino]acetyl]-3-azabicyclo[3.1.0]hexane-2-carboxamide

To a solution of (1R,2S,5S)—N-[(1S)-2-amino-1-[[(3R)-5,5-dimethyl-2-oxo-pyrrolidin-3-yl]methyl]-2-oxo-ethyl]-6,6-dimethyl-3-[2-(1-methylcyclopropyl)-2-[(2,2,2-trifluoroacetyl)amino]acetyl]-3-azabicyclo[3.1.0]hexane-2-carboxamide (400 mg, 735.87 umol, 1 eq) in DCM (10 mL) was added burgess reagent (526.08 mg, 2.21 mmol, 3 eq). The mixture was stirred at 25° C. for 2 h. Upon completion, the reaction mixture was diluted with H₂O (30 mL) and extracted with ethyl acetate 60 mL (20 mL*3). The combined organic layers were washed with brine 100 mL (20 mL*5), dried over Na₂SO₄, concentrated in vacuum. The residue was purified by prep-HPLC (neutral condition, column:Waters Xbridge Prep OBD C18 150*40 mm*10 um; mobile phase: [water(NH₄HCO₃)-ACN]; B %:25%-65%, 8 min) to give (1R,2S,5S)—N-[(1S)-1-cyano-2-[(3R)-5,5-dimethyl-2-oxo-pyrrolidin-3-yl]ethyl]-6,6-dimethyl-3-[2-(1-methylcyclopropyl)-2-[(2,2,2-trifluoroacetyl)amino] acetyl]-3-azabicyclo[3.1.0]hexane-2-carboxamide (219.4 mg, 417.46 umol, 56.73% yield, 100% purity) as a white solid. MS (ESI) m/z 526.3 [M+H]⁺.

¹H NMR (400 MHz, DMSO-d₆) δ=9.58 (br d, J=6.2 Hz, 1H), 8.99 (d, J=8.2 Hz, 1H), 7.82 (s, 1H), 4.91 (ddd, J=6.1, 8.2, 9.7 Hz, 1H), 4.34 (d, J=6.1 Hz, 1H), 4.12 (s, 1H), 3.84-3.78 (m, 1H), 3.68 (d, J=10.7 Hz, 1H), 2.58-2.52 (m, 1H), 2.17-2.08 (m, 1H), 1.99 (dd, J=8.8, 12.3 Hz, 1H), 1.73 (ddd, J=6.1, 10.2, 13.6 Hz, 1H), 1.64-1.47 (m, 2H), 1.28 (d, J=7.5 Hz, 1H), 1.18 (s, 3H), 1.12 (s, 3H), 1.07 (s, 3H), 1.03 (s, 3H), 0.92 (s, 3H), 0.81 (br dd, J=4.5, 9.4 Hz, 1H), 0.73 (br dd, J=4.6, 9.2 Hz, 1H), 0.35-0.22 (m, 2H).

Example 63: Synthesis of (1R,2S,5S)—N-[(1S)-1-cyano-2-[(3R)-5,5-dimethyl-2-oxo-pyrrolidin-3-yl]ethyl]-6,6-dimethyl-3-[2-(1-methylcyclopropyl)-2-[(2,2,2-trifluoroacetyl)amino]acetyl]-3-azabicyclo[3.1.0]hexane-2-carboxamide (Compound 340 Isomer 2)

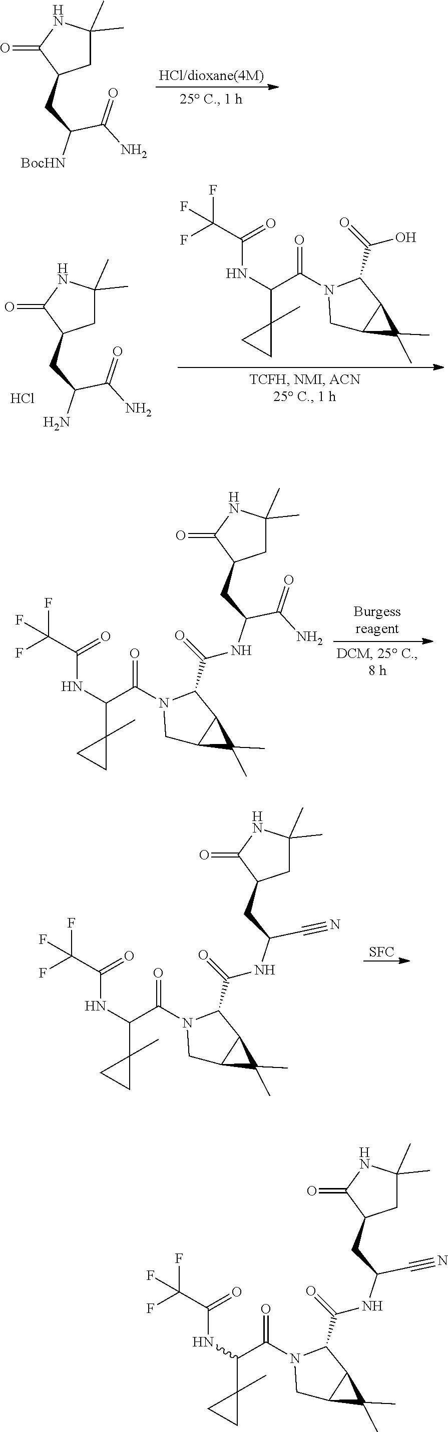

Step 1: (2S)-2-amino-3-[(3R)-5,5-dimethyl-2-oxo pyrrolidin-3-yl]propanamide

A mixture of tert-butyl N-[(1S)-2-amino-1-[[(3R)-5,5-dimethyl-2-oxo-pyrrolidin-3-yl]methyl]-2-oxo-ethyl]carbamate (2.1 g, 7.01 mmol, 1 eq) in HCl/dioxane (4 M, 30 mL, 17.11 eq) was stirred at 25° C. for 1 h. Upon completion, the reaction mixture was concentrated under reduced pressure to give (2S)-2-amino-3-[(3R)-5,5-dimethyl-2-oxo-pyrrolidin-3-yl]propanamide(1.6 g, crude, HCl) as a white solid.

Step 2: (1R,2S,5S)—N-[(1S)-2-amino-1-[[(3R)-5,5-dimethyl-2-oxo pyrrolidin-3-yl]methyl]-2-oxo-ethyl]-6,6-dimethyl-3-[2-(1-methylcyclopropyl)-2-[(2,2,2-trifluoroacetyl)amino]acetyl]-3-azabicyclo[3.1.0]hexane-2-carboxamide

To a solution of (2S)-2-amino-3-[(3R)-5,5-dimethyl-2-oxo-pyrrolidin-3-yl]propanamide (1.63 g, 6.90 mmol, 1 eq, HCl) and (1R,2S,5S)-6,6-dimethyl-3-[2-(1-methylcyclopropyl)-2-[(2,2,2-trifluoroacetyl)amino]acetyl]-3-azabicyclo[3.1.0]hexane-2-carboxylic acid (2 g, 5.52 mmol, 0.8 eq) in ACN (50 mL) was added 1-methylimidazole (1.70 g, 20.70 mmol, 1.65 mL, 3 eq) and [chloro(dimethylamino)methylene]-dimethyl-ammonium; hexafluorophosphate (3.87 g, 13.80 mmol, 2 eq). The mixture was stirred at 25° C. for 1 h. Upon completion, the reaction mixture was diluted with H₂O 70 mL and extracted with ethyl acetate 180 mL (60 mL*3). The combined organic layers were washed with brine 90 mL (90 mL*1), dried over Na₂SO₄, filtered and concentrated under reduced pressure to give the crude product. The crude was purified by column chromatography (SiO₂, Petroleum ether:Ethyl acetate=10:1 to EA:MeOH=50:1) to give (1R,2S,5S)—N-[(1S)-2-amino-1-[[(3R)-5,5-dimethyl-2-oxo-pyrrolidin-3-yl]methyl]-2-oxo-ethyl]-6,6-dimethyl-3-[2-(1-methylcyclopropyl)-2-[(2,2,2-trifluoroacetyl)amino]acetyl]-3-azabicyclo[3.1.0]hexane-2-carboxamide (3.9 g, crude) as a yellow oil. MS (ESI) m/z 544.3 [M+H]⁺

Step 3: (1R,2S,5S)—N-[(1S)-1-cyano-2-[(3R)-5,5-dimethyl-2-oxo pyrrolidin-3-yl]ethyl]-6,6-dimethyl-3-[2-(1-methylcyclopropyl)-2-[(2,2,2-trifluoroacetyl)amino]acetyl]-3-azabicyclo[3.1.0]hexane-2-carboxamide

A mixture of (1R,2S,5S)—N-[(1S)-2-amino-1-[[(3R)-5,5-dimethyl-2-oxo-pyrrolidin-3-yl]methyl]-2-oxo-ethyl]-6,6-dimethyl-3-[2-(1-methylcyclopropyl)-2-[(2,2,2-trifluoroacetyl)amino]acetyl]-3-azabicyclo[3.1.0]hexane-2-carboxamide (3.90 g, 7.17 mmol, 1 eq) in DCM (100 mL) was added burgess reagent (10.26 g, 43.05 mmol, 6 eq) and stirred at 25° C. for 16 h. Upon completion, the reaction mixture was quenched by addition H₂O 100 mL at 25° C., and then extracted with DCM 300 mL (100 mL*3). The combined organic layers were washed with brine 150 mL (150 mL*1), dried over Na₂SO₄, filtered and concentrated under reduced pressure to give the crude product. The crude was purified by prep-HPLC (neutral condition; column: Waters Xbridge BEH C18 250*70 mm*10 um; mobile phase: [water(NH₄HCO₃)-ACN]; B %: 35%-65%, 20 min) to give (1R,2S,5S)—N-[(1S)-1-cyano-2-[(3R)-5,5-dimethyl-2-oxo-pyrrolidin-3-yl]ethyl]-6,6-dimethyl-3-[2-(1-methylcyclopropyl)-2-[(2,2,2-trifluoroacetyl)amino]acetyl]-3-azabicyclo[3.1.0]hexane-2-carboxamide(580 mg) as a white solid. MS (ESI) m/z 526.5 [M+H]⁺

Step 4: (1R,2S,5S)—N-[(1S)-1-cyano-2-[(3R)-5,5-dimethyl-2-oxo pyrrolidin-3-yl]ethyl]-6,6-dimethyl-3-[2-(1-methylcyclopropyl)-2-[(2,2,2-trifluoroacetyl)amino]acetyl]-3-azabicyclo[3.1.0]hexane-2-carboxamide

(1R,2S,5S)—N-[(1S)-1-cyano-2-[(3R)-5,5-dimethyl-2-oxo-pyrrolidin-3-yl]ethyl]-6,6-dimethyl-3-[2-(1-methylcyclopropyl)-2-[(2,2,2-trifluoroacetyl)amino]acetyl]-3-azabicyclo[3.1.0]hexane-2-carboxamide was further purified by SFC(column: REGIS(S,S)WHELK-O 1(250 mm*25 mm,10 um); mobile phase: [Neu-IPA]; B %: 30%-30%, 7 min) to give (1R,2S,5S)—N-[(1S)-1-cyano-2-[(3R)-5,5-dimethyl-2-oxo-pyrrolidin-3-yl]ethyl]-6,6-dimethyl-3-[2-(1-methylcyclopropyl)-2-[(2,2,2-trifluoroacetyl)amino]acetyl]-3-azabicyclo[3.1.0]hexane-2-carboxamide (0.22 g, 418.60 umol, 5.83% yield, 100% purity) as a white solid. MS (ESI) m/z 526.5 [M+H]⁺

¹H NMR (400 MHz, DMSO-d₆) δ=9.95-9.48 (m, 1H), 9.26-8.67 (m, 1H), 7.91-7.75 (m, 1H), 4.98-4.84 (m, 1H), 4.42-4.21 (m, 1H), 4.19-4.04 (m, 1H), 3.93-3.48 (m, 2H), 2.65-2.54 (m, 1H), 2.28-1.93 (m, 2H), 1.87-1.68 (m, 1H), 1.63-1.41 (m, 2H), 1.39-1.29 (m, 1H), 1.25-0.80 (m, 16H), 0.50-0.14 (m, 3H)

¹H NMR (400 MHz, ACETONITRILE-d3) δ=8.50-8.00 (m, 1H), 7.81-7.63 (m, 1H), 6.46-6.12 (m, 1H), 4.79 (td, J=6.9, 9.9 Hz, 1H), 4.43-4.12 (m, 2H), 3.96 (dd, J=5.4, 10.5 Hz, 1H), 3.64 (d, J=10.3 Hz, 1H), 2.72-2.55 (m, 1H), 2.25-2.18 (m, 1H), 2.14 2.10 (m, 1H), 1.93-1.85 (m, 1H), 1.70-1.41 (m, 3H), 1.29-1.16 (m, 6H), 1.13-0.91 (m, 9H), 0.87-0.77 (m, 1H), 0.62-0.28 (m, 3H)

Example 64: Synthesis of (1R,2S,5S)—N-[(1S)-1-cyano-2-[(3R)-5,5-dimethyl-2-oxo-pyrrolidin-3-yl]ethyl]-6,6-dimethyl-3-[2-(1-methylcyclobutyl)-2-[(2,2,2-trifluoroacetyl)amino]acetyl]-3-azabicyclo[3.1.0]hexane-2-carboxamide (Compound 342)

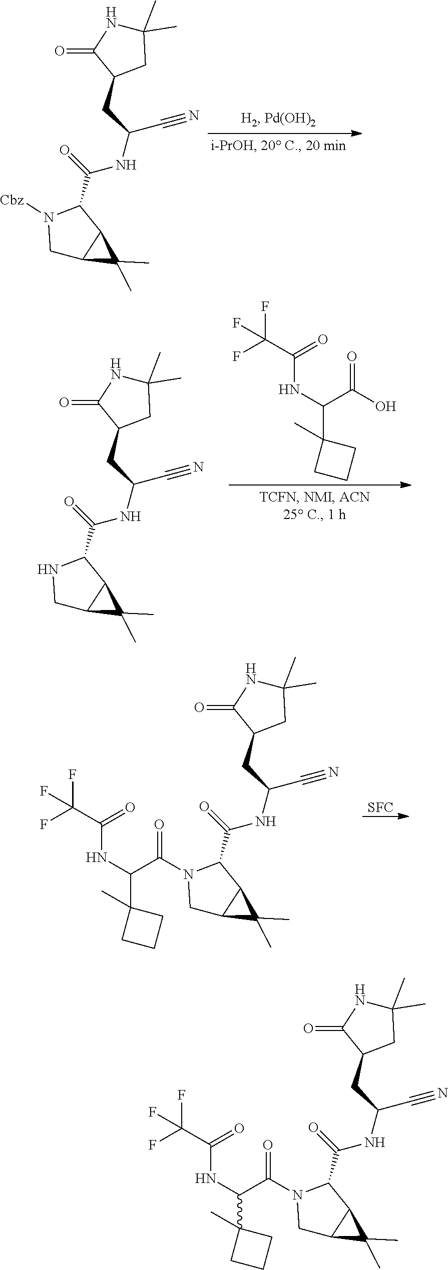

Step 1: (1R,2S,5S)—N-[(1S)-1-cyano-2-[(3R)-5,5-dimethyl-2-oxo pyrrolidin-3-yl]ethyl]-6,6-dimethyl-3-azabicyclo[3.1.0]hexane-2-carboxamide

To a solution of benzyl (1R,2S,5S)-2-[[(1S)-1-cyano-2-[(3R)-5,5-dimethyl-2-oxo-pyrrolidin-3-yl]ethyl]carbamoyl]-6,6-dimethyl-3-azabicyclo[3.1.0]hexane-3-carboxylate (4 g, 8.84 mmol, 1 eq) in i-PrOH (20 mL) was added Pd(OH)₂ (1.24 g, 1.77 mmol, 20% purity, 0.2 eq) under H₂ (15 psi). The mixture was stirred at 20° C. for 20 min. Upon completion, The reaction mixture was filtered and concentrated in vacuum to give (1R,2S,5S)—N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (2.3 g, crude) as a white solid. MS (ESI) m/z 319.3 [M+H]⁺

Step 2: (1R,2S,5S)—N-[(1S)-1-cyano-2-[(3R)-5,5-dimethyl-2-oxo pyrrolidin-3-yl]ethyl]-6,6-dimethyl-3-[2-(1-methylcyclobutyl)-2-[(2,2,2-trifluoroacetyl)amino]acetyl]-3-azabicyclo[3.1.0]hexane-2-carboxamide

To a solution of (1R,2S,5S)—N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (2.3 g, 7.22 mmol, 1 eq) in ACN (20 mL) was added 2-(1-methylcyclobutyl)-2-[(2,2,2-trifluoroacetyl)amino]acetic acid (1.75 g, 7.32 mmol, 1 eq) and NMI (1.80 g, 21.95 mmol, 1.75 mL, 3 eq) and then was added TCFH (4.11 g, 14.63 mmol, 2 eq). The mixture was stirred at 25° C. for 1 h. Upon completion, the mixture was diluted with H₂O (60 mL) and then was extracted with ethyl acetate (30 mL*3). The combined organic layers were dried over Na₂SO₄, filtered and concentrated in vacuum and was purified by prep-HPLC (column: Xtimate C18 10u 250 mm*80 mm; mobile phase: [water (NH₄HCO₃)-ACN]; B %: 30%-60%, 32 min) to give (1R,2S,5S)—N-[(1S)-1-cyano-2-[(3R)-5,5-dimethyl-2-oxo-pyrrolidin-3-yl]ethyl]-6,6-dimethyl-3-[2-(1-methylcyclobutyl)-2-[(2,2,2-trifluoroacetyl)amino]acetyl]-3-azabicyclo[3.1.0]hexane-2-carboxamide (1.7 g, 3.06 mmol, 41.77% yield, 97% purity) as white solid. MS (ESI) m/z 540.3 [M+H]⁺

Step 3: (1R,2S,5S)—N-[(1S)-1-cyano-2-[(3R)-5,5-dimethyl-2-oxo pyrrolidin-3-yl]ethyl]-6,6-dimethyl-3-[2-(1-methylcyclobutyl)-2-[(2,2,2-trifluoroacetyl)amino Jacetyl]-3-azabicyclo[3.1.0]hexane-2-carboxamide

The (1R,2S,5S)—N-[(1S)-1-cyano-2-[(3R)-5,5-dimethyl-2-oxo-pyrrolidin-3-yl]ethyl]-6,6-dimethyl-3-[2-(1-methylcyclobutyl)-2-[(2,2,2-trifluoroacetyl)amino]acetyl]-3-azabicyclo[3.1.0]hexane-2-carboxamide (700 mg, 1.30 mmol, 1 eq) was separated by SFC (column: DAICEL CHIRALPAK IC (250 mm*30 mm, 10 um); mobile phase: [Neu-IPA]; B %: 18%-18%, 8 min) to give product (1R,2S,5S)—N-[(1S)-1-cyano-2-[(3R)-5,5-dimethyl-2-oxo-pyrrolidin-3-yl]ethyl]-6,6-dimethyl-3-[2-(1-methylcyclobutyl)-2-[(2,2,2-trifluoroacetyl)amino]acetyl]-3-azabicyclo[3.1.0]hexane-2-carboxamide Isomer 1 (125.49 mg, 232.57 umol, 17.93% yield, 100% purity) as a white solid. MS (ESI) m/z 540.2 [M+H]⁺

¹H NMR (400 MHz, DMSO-d₆) δ=9.59 (d, J=5.6 Hz, 1H), 8.94 (d, J=8.2 Hz, 1H), 7.83 (s, 1H), 4.92 (ddd, J=6.0, 8.2, 9.8 Hz, 1H), 4.42 (br d, J=5.4 Hz, 1H), 4.15 (s, 1H), 3.84 (dd, J=5.6, 10.4 Hz, 1H), 3.58 (d, J=10.4 Hz, 1H), 2.60-2.51 (m, 1H), 2.41-2.30 (m, 1H), 2.21-2.04 (m, 2H), 2.00 (dd, J=8.6, 12.2 Hz, 1H), 1.94-1.81 (m, 1H), 1.75 (ddd, J=6.0, 10.0, 13.6 Hz, 1H), 1.68-1.59 (m, 1H), 1.58-1.47 (m, 4H), 1.29 (d, J=7.6 Hz, 1H), 1.20 (d, J=14.4 Hz, 6H), 1.14-1.08 (m, 3H), 1.07-0.99 (m, 3H), 0.87 (s, 3H).

(1R,2S,5S)—N-[(1S)-1-cyano-2-[(3R)-5,5-dimethyl-2-oxo-pynrolidin-3-yl]ethyl]-6,6-dimethyl-3-[2-(1-methylcyclobutyl)-2-[(2,2,2-trifluoroacetyl)amino]acetyl]-3-azabicyclo[3.1.0]hexane-2-carboxamide Isomer 2 (148.54 mg, 260.14 umol, 20.05% yield, 94.5% purity) was obtained as a white solid. MS (ESI) m/z 540.2 [M+H]⁺

¹H NMR (400 MHz, DMSO-d₆) δ=10.15-9.53 (m, 1H), 9.28-8.76 (m, 1H), 7.85 (d, J=4.8 Hz, 1H), 5.01-4.79 (m, 1H), 4.18-4.62 (d, J=8.8 Hz, 2H), 3.84-3.42 (m, 2H), 2.73-2.57 (m, 1H), 2.63-2.53 (m, 1H), 2.30-1.72 (m, 5H), 1.70-1.42 (m, 5H), 1.40 1.35 (m, 1H), 1.20-1.16 (m, 6H), 1.15-1.07 (m, 3H), 1.02 (d, J=6.8 Hz, 3H), 0.96-0.80 (m, 3H).

¹H NMR (400 MHz, CD3CN) δ=8.59-7.90 (m, 2H), 6.37 (s, 1H), 4.87-4.66 (m, 1H), 4.61 (d, J=7.6 Hz, 1H), 4.36-4.14 (m, 1H), 3.98 (dd, J=5.6, 10.4 Hz, 1H), 3.69 3.53 (m, 1H), 2.79-2.61 (m, 1H), 2.17-2.12 (m, 2H), 2.08 (s, 2H), 1.95-1.92 (m, 1H), 1.82-1.73 (m, 1H), 1.71-1.53 (m, 4H), 1.57-1.46 (m, 2H), 1.29 (d, J=3.2 Hz, 6H), 1.23 (s, 3H), 1.07-1.04 (m, 3H), 0.98 (s, 3H).

Example 65: Synthesis of (1R,2S,5S)—N-[(1S)-1-cyan-2-[(6R)-5-oxo-4-azaspiro[2.4]heptan-6-yl]ethyl]-6,6-dimethyl-3-[2-(1-methylcyclobutyl)-2-[(2,2,2-trifluoroacetyl)amino]acetyl]-3-azabicyclo[3.1.0]hexane-2-carboxamide (Compound 343)

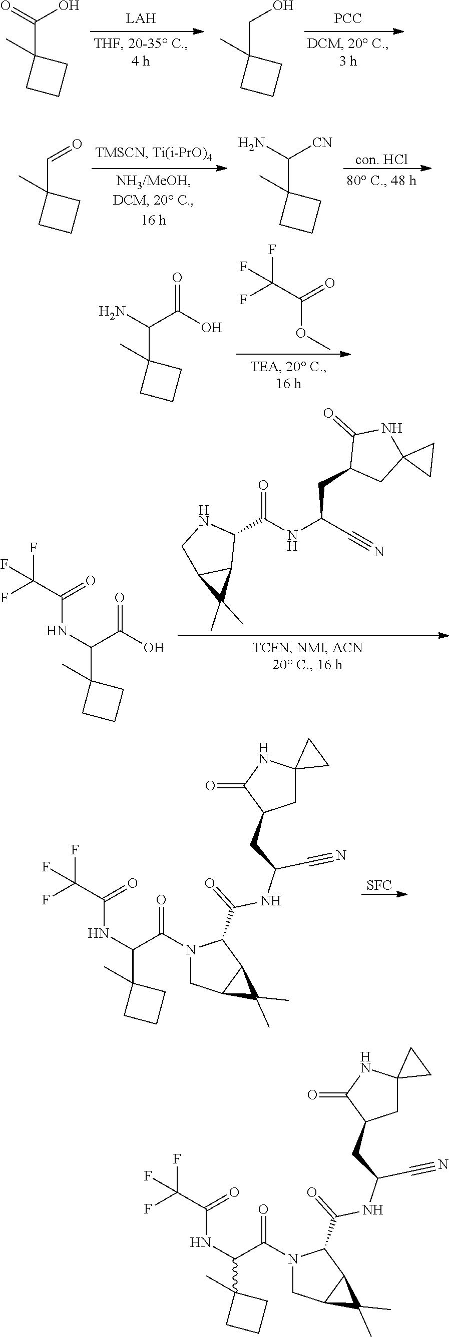

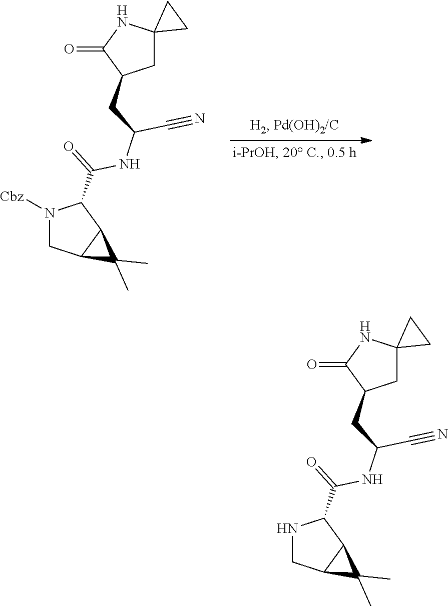 [SMILES: CC1(C)[C@@H]2[C@@H](C(=O)N[C@H](C#N)C[C@@H]3CC4(CC4)NC3=O)N(C(=O)OCc3ccccc3)C[C@@H]21 ; CC1(C)[C@@H]2[C@@H](C(=O)N[C@H](C#N)C[C@@H]3CC4(CC4)NC3=O)NC[C@@H]21]

Step 1: (1-methylcyclobutyl)methanol

Under N₂, LAH (6.65 g, 175.22 mmol, 1 eq) was slowly added to 1-methylcyclobutanecarboxylic acid (20 g, 175.22 mmol, 1 eq) in THE (600 mL) at 20° C., then the mixture was raised to 35° C. and the mixture was stirred for 4 h. Upon completion the mixture was cooled to 0° C., quenched with H₂O (6.6 mL), 15% NaOH aq. (6.6 mL), H₂O(19.8 mL), The mixture was stirred for 16 h and filtered with a celice cake and the solution was concentrated under reduced pressure to give (1-methylcyclobutyl)methanol (15 g, 119.81 mmol, 68.38% yield, 80% purity) as light yellow oil.

Step 2: 1-methylcyclobutanecarbaldehyde

PCC (40.35 g, 187.20 mmol, 1.25 eq) and same weight of celite were taken up as a suspension in DCM (440 mL). To this was added dropwise of (1-methylcyclobutyl)methanol (15 g, 149.76 mmol, 1 eq) in DCM (66 mL) and the mixture was stirred at 20° C. for 3 h. Upon completion, the mixture was filtered with a celite cake. The filtrate (as light yellow liquid; 1-methylcyclobutanecarbaldehyde (14 g, 142.65 mmol, 95.25% yield) was used for next step without further work up and purification.

Step 3: 2-amino-2-(1-methylcyclobutyl)acetonitrile

To a mixture of 1-methylcyclobutanecarbaldehyde (14 g, 142.65 mmol, 1 eq) in DCM (600 mL) was added NH₃/MeOH (7 M, 81.51 mL, 4 eq) and Ti(Oi-Pr)₄ (40.54 g, 142.65 mmol, 42.10 mL, 1 eq), the mixture was stirred at 20° C. for 1 h. then TMSCN (42.46 g, 427.95 mmol, 53.54 mL, 3 eq) was added, the mixture was stirred at 20° C. for 15 h. Upon completion, the mixture was quenched with water (500 mL), yellow solid separated, filtered with a celice cake, the organic layer was separated and the washed with brine (500 mL), dried with Na₂SO₄, filtered and concentrated to give the residue. The residue was purified by column chromatography (SiO₂, Petroleum ether/Ethyl acetate=5/1 to 3/1) to give 2-amino-2-(1-methylcyclobutyl)acetonitrile (10 g, 72.47 mmol, 50.81% yield, 90% purity) as light yellow oil.

Step 4: 2-amino-2-(1-methylcyclobutyl)acetic acid

In a sealed tube, 2-amino-2-(1-methylcyclobutyl)acetonitrile (5 g, 40.26 mmol, 1 eq) in 12 M HCl (100 mL) was stirred at 80° C. for 48 h. Upon completion, the mixture was concentrated to give 2-amino-2-(1-methylcyclobutyl)acetic acid (7 g, 35.07 mmol, 87.10% yield, 90% purity, HCl) as white solid.

Step 5: 2-(1-methylcyclobutyl)-2-[(2,2,2-trifluoroacetyl)amino]acetic acid

A mixture of methyl 2,2,2-trifluoroacetate (35.64 g, 278.33 mmol, 28.06 mL, 25 eq) in 2-amino-2-(1-methylcyclobutyl)acetic acid (2 g, 11.13 mmol, 1 eq, HCl) was added Et₃N (3.38 g, 33.40 mmol, 4.65 mL, 3 eq), and the mixture was stirred at 20° C. for 16 h. Upon completion, the reaction mixture was concentrated under reduced pressure to remove methyl 2,2,2-trifluoroacetate. The residue was diluted with 0.5M HCl 20 mL and extracted with DCM (20 mL*3). The combined organic layers were washed with brine 50 mL, dried over Na₂SO₄, filtered and concentrated under reduced pressure to give 2-(1-methylcyclobutyl)-2-[(2,2,2-trifluoroacetyl)amino]acetic acid (3.3 g, 8.28 mmol, 74.35% yield, 60% purity) as light yellow oil.

Step 6: (1R,2S,5S)—N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide

To a solution of benzyl (1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carboxylate (3 g, 6.66 mmol, 1 eq) in i-PrOH (60 mL) was added Pd(OH)₂/C (703.13 mg, 5.01 mmol, 7.52e-1 eq) under N₂. The suspension was degassed under vacuum and purged with H₂ several times. The mixture was stirred under H₂ (15 psi) at 20° C. for 0.5 h. Upon completion, the reaction mixture was filtered and the filter was concentrated to give (1R,2S,5S)—N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (2.2 g, crude) as a white solid, which was used for next step without further purification.

Step 7: benzyl N-[(1S)-1-[[2-[[(1S)-2-amino-2-oxo-1-[[(6R)-5-oxo-4-azaspiro[2.4]heptan-6-yl]methyl]ethyl]amino]-2-oxo-1-(trimethylsilylmethyl)ethyl]carbamoyl]-2,2-dimethyl-propyl]carbamate

To a solution of (1R,2S,5S)—N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (1.2 g, 3.79 mmol, 1 eq), 2-(1-methylcyclobutyl)-2-[(2,2,2-trifluoroacetyl)amino]acetic acid (1.2 g, 3.01 mmol, 60% purity, 7.94e-1 eq) in ACN (40 mL) was added 1-methylimidazole (934.14 mg, 11.38 mmol, 906.93 uL, 3 eq) and TCFH (1.60 g, 5.69 mmol, 1.5 eq), the mixture was stirred at 20° C. for 16 h. Upon completion, the reaction mixture was quenched by addition H₂O 50 mL at 20° C., and then diluted with EA 50 mL and extracted with EA (50 mL*2). The combined organic layers were washed with brine 100 mL, dried over Na₂SO₄, filtered and concentrated under reduced pressure to give a residue. The residue was purified by prep-HPLC (column: Welch Xtimate C18 250*70 mm #IOum; mobile phase: [water(NH₄HCO₃)-ACN]; B %: 40%-70%, 20 min;) to give (1R,2S,5S)—N-[(1S)-1-cyano-2-[(6R)-5-oxo-4-azaspiro[2.4]heptan-6-yl]ethyl]-6,6-dimethyl-3-[2-(1-methylcyclobutyl)-2-[(2,2,2-trifluoroacetyl)amino]acetyl]-3-azabicyclo[3.1.0]hexane-2-carboxamide (1 g, 1.86 mmol, 49.05% yield) as white solid, 0.5 g of the product was SFC separated (column: DAICEL CHIRALPAK AD(250 mm*30 mm,10 um;); mobile phase: [Neu-ETOH]; B %: 18%-18%, 5 min) to give (1R,2S,5S)—N-[(1S)-1-cyano-2-[(6R)-5-oxo-4-azaspiro[2.4]heptan-6-yl]ethyl]-6,6-dimethyl-3-[2-(1-methylcyclobutyl)-2-[(2,2,2-trifluoroacetyl)amino]acetyl]-3-azabicyclo[3.1.0]hexane-2-carboxamide (110 mg, 197.03 umol, 5.20% yield, 96.29% purity) Isomer 1 as a white solid MS (ESI) m/z 538.3 [M+H]⁺

¹H NMR (400 MHz, ACETONITRILE-d3) S=8.23-7.85 (m, 1H), 7.81 (br d, J=6.8 Hz, 1H), 6.15 (br s, 1H), 4.75 (td, J=6.6, 10.0 Hz, 1H), 4.58 (d, J=7.8 Hz, 1H), 4.15 (s, 1H), 3.97 (dd, J=5.5, 10.5 Hz, 1H), 3.59 (d, J=10.5 Hz, 1H), 2.79-2.65 (m, 1H), 2.27 2.16 (m, 2H), 2.14-2.07 (m, 2H), 2.07-1.96 (m, 3H), 1.82-1.59 (m, 3H), 1.56-1.41 (m, 2H), 1.26 (s, 3H), 1.04 (s, 3H), 0.96 (s, 3H), 0.86-0.79 (m, 1H), 0.75-0.56 (m, 3H)

(1R,2S,5S)—N-[(1S)-1-cyano-2-[(6R)-5-oxo-4-azaspiro[2.4]heptan-6-yl]ethyl]-6,6-dimethyl-3-[2-(1-methylcyclobutyl)-2-[(2,2,2-trifluoroacetyl)amino]acetyl]-3-azabicyclo[3.1.0]hexane-2-carboxamide (160 mg, 297.63 umol, 7.85% yield, 100% purity) Isomer 2 was obtained as a white solid. MS (ESI) m/z 538.3 [M+H]⁺

¹H NMR (400 MHz, ACETONITRILE-d3) δ=7.77 (br d, J=7.3 Hz, 1H), 7.50 (br d, J=7.5 Hz, 1H), 6.17 (br s, 1H), 4.86 (ddd, J=5.9, 7.3, 10.3 Hz, 1H), 4.51 (d, J=8.3 Hz, 1H), 4.15 (s, 1H), 3.91 (dd, J=5.6, 10.3 Hz, 1H), 3.70 (d, J=10.4 Hz, 1H), 2.78-2.61 (m, 1H), 2.38-2.17 (m, 3H), 2.14-1.95 (m, 4H), 1.78-1.65 (m, 1H), 1.64-1.48 (m, 3H), 1.40 (d, J=7.6 Hz, 1H), 1.27 (s, 3H), 1.04 (s, 3H), 0.90 (s, 3H), 0.87-0.77 (m, 1H), 0.74 0.55 (m, 3H)

Example ___: Synthesis of (1R,2S,5S)-3-[2-(1-bicydo[1.1.1]pentanyl)-2-[(2,2,2-trifluoroacetyl)amino]acetyl]-N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (Compound 344)

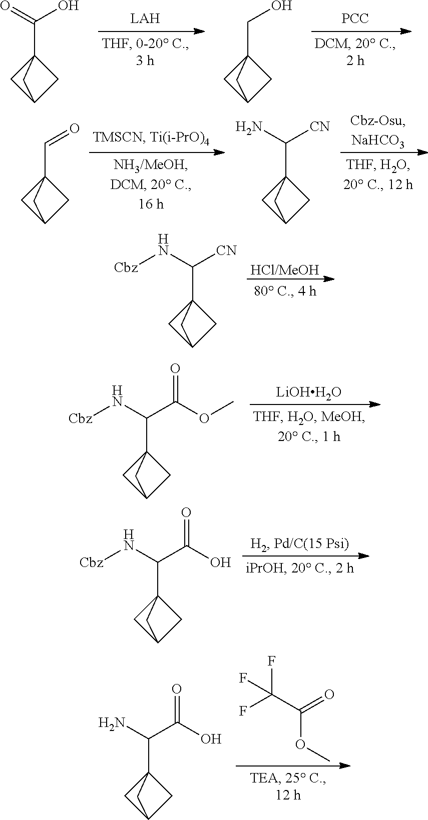

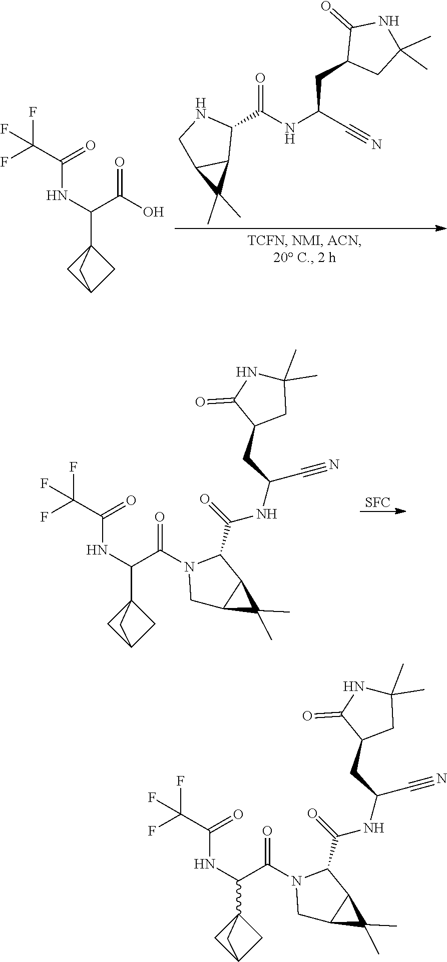

Step 1: 1-bicyclo[1.1.1]pentanylmethanol

To a solution of bicyclo[1.1.1]pentane-1-carboxylic acid (10 g, 89.19 mmol, 1 eq) in THE (100 mL) was added LAH (5.08 g, 133.78 mmol, 1.5 eq) at 0° C. under N₂. The mixture was stirred at 20° C. for 3 h under N₂ atmosphere. Upon completion, the reaction mixture was quenched by addition H₂O (5 mL) at 0° C., and then the solution 15% NaOH (5 mL) was added drop-wise, followed by H₂O (15 mL), then filtered and concentrated under reduced pressure to give I-bicyclo[1.1.1]pentanylmethanol (9 g, crude) as a colorless oil.

Step 2: bicyclo[1.1.1]pentane-1-carbaldehyde

To a solution of 1-bicyclo[1.1.1]pentanylmethanol (9 g, 91.70 mmol, 1 eq) in DCM (100 mL) was added PCC (29.65 g, 137.55 mmol, 1.5 eq) at 0° C. The mixture was stirred at 20° C. for 2 h. Upon completion, the reaction mixture was filtered and the filtrate was used to the next step directly. To give bicyclo[1.1.1]pentane-1-carbaldehyde (9 g, crude) in DCM (100 mL) as a brown liquid.

Step 3: 2-amino-2-(1-bicyclo[1.1.1]pentanyl)acetonitrile

To a solution of bicyclo[1.1.1]pentane-1-carbaldehyde (9 g, 93.63 mmol, 1 eq) in DCM (120 mL) was added NH₃/MeOH (7 M, 66.88 mL, 5 eq) and Ti(i-PrO)₄ (39.91 g, 140.44 mmol, 41.45 mL, 1.5 eq), the solution was stirred at 20° C. for 1 h before TMSCN (18.58 g, 187.25 mmol, 23.43 mL, 2 eq) was added. The mixture was stirred at 20° C. for 12 h. Upon completion, the reaction mixture was diluted with water (300 mL) and then filtered, the filtrate was extracted with DCM (100 mL*3). The combined organic layers were dried over Na₂SO₄, filtered and concentrated under reduced pressure to give a residue. The residue was purified by column chromatography (SiO₂, Petroleum ether:Ethyl acetate=10:1 to 1:1) to give 2-amino-2-(1-bicyclo[1.1.1]pentanyl)acetonitrile (8 g, 49.11 mmol, 52.46% yield, 75% purity) as a yellow oil.

Step 4: benzyl N-[1-bicyclo[1.1.1]pentanyl(cyano)methyl]carbamate

To a solution of 2-amino-2-(1-bicyclo[1.1.1]pentanyl)acetonitrile (8 g, 65.48 mmol, 1 eq) in THE (50 mL) was added benzyl (2,5-dioxopyrrolidin-1-yl) carbonate (19.58 g, 78.58 mmol, 1.2 eq), and then NaHCO₃(16.50 g, 196.45 mmol, 7.64 mL, 3 eq) in H₂O (100 mL) was added. The mixture was stirred at 20° C. for 12 h under N₂ atmosphere. Upon completion, the reaction mixture was diluted with H₂O (200 mL) and extracted with EA (100 mL*3). The combined organic layers were dried over Na₂SO₄, filtered and concentrated under reduced pressure to give a residue. The residue was purified by column chromatography (SiO₂, Petroleum ether:Ethyl acetate=20:1 to 10:1) to give benzyl N-[1-bicyclo[1.1.1]pentanyl(cyano)methyl]carbamate (5.5 g, 19.31 mmol, 29.49% yield, 90% purity) as a yellow oil.

Step 5: methyl 2-(benzyloxycarbonylamino)-2-(1-bicyclo(1.1.1]pentanyl)acetate

A solution of benzyl N-[1-bicyclo[1.1.1]pentanyl(cyano)methyl]carbamate (5.5 g, 21.46 mmol, 1 eq) in HCl/MeOH (4 M, 65 mL, 12.12 eq) was stirred at 80° C. for 4 h. Upon completion, the reaction mixture was concentrated under reduced pressure to give a residue to give methyl 2-(benzyloxycarbonylamino)-2-(1-bicyclo[1.1.1]pentanyl)acetate (6 g, crude) as a white solid.

Step 6: 2-(benzyloxycarbonylamino)-2-(1-bicyclo(1.1.1]pentanyl)acetic acid

To a solution of methyl 2-(benzyloxycarbonylamino)-2-(1-bicyclo[1.1.1]pentanyl)acetate (6 g, 20.74 mmol, 1 eq) in THE (40 mL), H₂O (7 mL) and MeOH (7 mL) was added LiOH.H₂O (2.61 g, 62.21 mmol, 3 eq). The mixture was stirred at 20° C. for 2 h. Upon completion, the reaction mixture was concentrated under reduced pressure to give 2-(benzyloxycarbonylamino)-2-(1-bicyclo[1.1.1]pentanyl)acetic acid (5.8 g, crude) as a white solid.

Step 7: 2-amino-2-(1-bicyclo[1.1.1]pentanyl)acetic acid

To a solution of 2-(benzyloxycarbonylamino)-2-(1-bicyclo[1.1.1]pentanyl)acetic acid (5.3 g, 19.25 mmol, 1 eq) in IPA (50 mL) was added Pd/C (0.1 g, 10% purity). The mixture was stirred at 20° C. for 2 h under H₂ (38.89 mg, 19.25 mmol, 1 eq) at 15 Psi. Upon completion, the reaction mixture was filtered and concentrated under reduced pressure to give 2-amino-2-(1-bicyclo[1.1.1]pentanyl)acetic acid (3 g, crude) as a white solid.

Step 8: 2-(1-bicyclo[1.1.1]pentanyl)-2-[(2,2,2-trifluoroacetyl)amino]acetic acid

To a solution of 2-amino-2-(1-bicyclo[1.1.1]pentanyl)acetic acid (3 g, 21.25 mmol, 1 eq) in methyl 2,2,2-trifluoroacetate (44.45 g, 347.13 mmol, 35 mL, 16.33 eq) was added TEA (6.45 g, 63.75 mmol, 8.87 mL, 3 eq). The mixture was stirred at 25° C. for 12 h. Upon completion, the reaction mixture was under reduced pressure and diluted with H₂O (50 mL) and acidified with aq. HCl (1 M) to adjust pH=4, and extracted with EA (30 mL*3). The combined organic layers were washed dried over Na₂SO₄, filtered and concentrated under reduced pressure to give 2-(1-bicyclo[1.1.1]pentanyl)-2-[(2,2,2-trifluoroacetyl)amino]acetic acid (5 g, crude) as a yellow oil.

Step 9: (1R,2S,5S)-3-[2-(1-bicyclo(1.1.1]pentanyl)-2-[(2,2,2-trifluoroacetyl)amino]acetyl]-N-[(1S)-1-cyano-2-[(3R)-5,5-dimethyl-2-oxo pyrrolidin-3-yl]ethyl]-6,6-dimethyl-3-azabicyclo[3.1.0]hexane-2-carboxamide

To a solution of 2-(1-bicyclo[1.1.1]pentanyl)-2-[(2,2,2-trifluoroacetyl)amino]acetic acid (1 g, 4.22 mmol, 1 eq) and (1R,2S,5S)—N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (1.34 g, 4.22 mmol, 1 eq) in ACN (15 mL) was added NMI (692.31 mg, 8.43 mmol, 672.15 uL, 2 eq) and TCFH (2.37 g, 8.43 mmol, 2 eq). The mixture was stirred at 20° C. for 2 h. Upon completion, the reaction mixture was diluted with water (5 mL) and extracted with EA (2 mL*3). The combined organic layers were dried over Na₂SO₄, filtered and concentrated under reduced pressure to give a residue. The residue was purified by prep-HPLC (column: Waters Xbridge BEH C18 250*50 mm*10 um; mobile phase: [water(NH₄HCO₃)-ACN]; B %: 30%-60%, 10 min) to give (1R,2S,5S)-3-[2-(1-bicyclo[1.1.1]pentanyl)-2-[(2,2,2-trifluoroacetyl)amino]acetyl]-N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (0.35 g, 390.64 umol, 9.27% yield, 60% purity) as a yellow solid.

Step 10: (1R,2S,5S)-3-[2-(1-bicyclo[1.1.1]pentanyl)-2-[(2,2,2-trifluoroacetyl)amino]acetyl]-N-[(1S)-1-cyano-2-[(3R)-5,5-dimethyl-2-oxo pyrrolidin-3-yl]ethyl]-6,6-dimethyl-3-azabicyclo[3.1.0]hexane-2-carboxamide

(1R,2S,5S)-3-[2-(1-bicyclo[1.1.1]pentanyl)-2-[(2,2,2-trifluoroacetyl)amino]acetyl]-N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (0.35 g, 651.07 umol, 1 eq) was separated by SFC(column: DAICEL CHIRALPAK AD(250 mm*30 mm,10 um); mobile phase: [Neu-IPA]; B %: 18%-18%, 7 min) to give (1R,2S,5S)-3-[2-(1-bicyclo[1.1.1]pentanyl)-2-[(2,2,2-trifluoroacetyl)amino]acetyl]-N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide Isomer 1 (153.05 mg, 281.86 umol, 43.29% yield, 99% purity) as a white solid. MS (ESI) m/z 538.3 [M+H]⁺

¹H NMR (400 MHz, DMSO-d₆) δ=9.39-9.16 (m, 1H), 8.83 (d, J=8.4 Hz, 1H), 7.80 (s, 1H), 4.98-4.83 (m, 1H), 4.79-4.29 (m, 1H), 4.27-4.06 (m, 1H), 3.97-3.72 (m, 1H), 3.51 (d, J=10.1 Hz, 1H), 3.31 (s, 1H), 2.67-2.52 (m, 1H), 2.19-2.06 (m, 1H), 2.04 1.88 (m, 1H), 1.80-1.73 (m, 3H), 1.71 (d, J=9.4 Hz, 3H), 1.63-1.41 (m, 2H), 1.32 (d, J=7.7 Hz, 1H), 1.22-1.06 (m, 6H), 1.06-0.77 (m, 7H).

(1R,2S,5S)-3-[2-(1-bicyclo[1.1.1]pentanyl)-2-[(2,2,2-trifluoroacetyl)amino]acetyl]-N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide Isomer 2 (126.43 mg, 232.83 umol, 35.76% yield, 99% purity) was obtained as a white solid. MS (ESI) m/z 538.3 [M+H]⁺

¹H NMR (400 MHz, DMSO-d₆) δ=9.28 (br d, J=4.8 Hz, 1H), 9.00 (d, J=8.2 Hz, 1H), 7.83 (s, 1H), 4.99-4.76 (m, 1H), 4.57-4.31 (m, 1H), 4.18 (s, 1H), 3.86-3.72 (m, 1H), 3.64 (d, J=10.4 Hz, 1H), 2.54 (br d, J=4.9 Hz, 1H), 2.45 (s, 1H), 2.23-2.09 (m, 1H), 2.07-1.95 (m, 1H), 1.90 (d, J=8.1 Hz, 3H), 1.74 (br d, J=8.7 Hz, 3H), 1.63-1.48 (m, 2H), 1.29 (d, J=7.6 Hz, 1H), 1.23-1.06 (m, 6H), 1.05-0.87 (m, 7H).

Example 66: Synthesis of (1R,2S,5S)-3-[2-(1-bicyclo[1.1.1]pentanyl)-2-[(2,2,2-trifluoroacetyl)amino]acetyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamid (Compound 345)

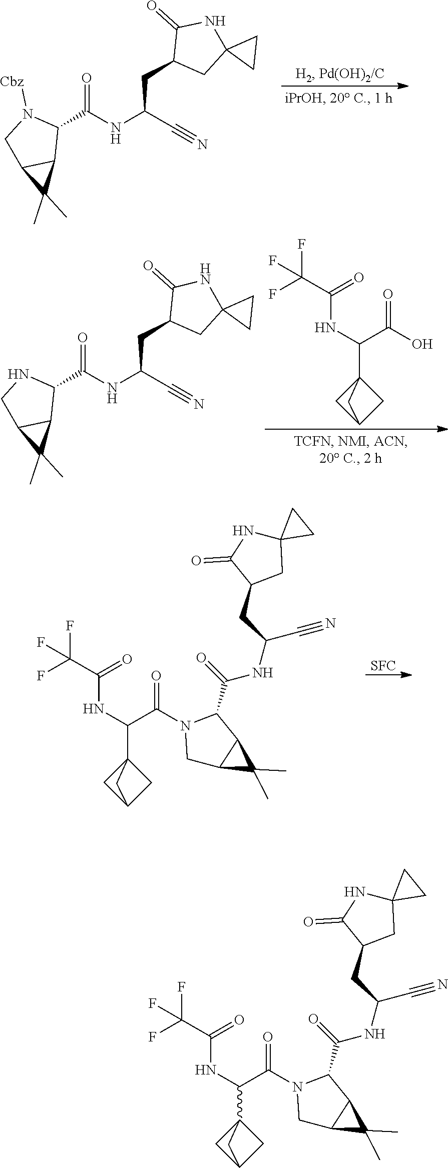

Step 1: (1R,2S,5S)—N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide

To a solution of benzyl (1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carboxylate (1 g, 2.22 mmol, 1 eq) in IPA (15 mL) was added Pd(OH)₂ (0.35 g, 498.43 umol, 20% purity, 2.25e-1 eq). The mixture was stirred at 20° C. for 1 h under H₂ (4.48 mg, 2.22 mmol, 1 eq) at 15 Psi. Upon completion, the reaction mixture was filtered and concentrated under reduced pressure to give a residue to give (1R,2S,5S)—N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (0.7 g, crude) as a white solid.

Step 2: (1R,2S,5S)-3-[2-(1-bicyclo(1.1.1]pentanyl)-2-[(2,2,2-trifluoroacetyl)amino]acetyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide

To a solution of 2-(1-bicyclo[1.1.1]pentanyl)-2-[(2,2,2-trifluoroacetyl)amino]acetic acid (1 g, 4.22 mmol, 1 eq) and (1R,2S,5S)—N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (0.7 g, 2.21 mmol, 5.25e-1 eq) in ACN (15 mL) was added NMI (692.31 mg, 8.43 mmol, 672.15 uL, 2 eq) and TCFH (2.37 g, 8.43 mmol, 2 eq). The mixture was stirred at 20° C. for 2 h. Upon completion, the reaction mixture was diluted with water (50 mL) and extracted with EA (20 mL*3). The combined organic layers were dried over Na₂SO₄, filtered and concentrated under reduced pressure to give a residue. The residue was purified by prep-HPLC (column: Waters Xbridge BEH C18 250*50 mm*10 um; mobile phase: [water(NH₄HCO₃)-ACN]; B %: 30%-60%, 10 min) to give (1R,2S,5S)-3-[2-(1-bicyclo[1.1.1]pentanyl)-2-[(2,2,2-trifluoroacetyl)amino]acetyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (0.45 g, 756.22 umol, 17.94% yield, 90% purity) as a yellow oil.

Step 3: (1R,2S,5S)-3-[2-(1-bicyclo(1.1.1]pentanyl)-2-[(2,2,2-trifluoroacetyl)amino]acetyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide

(1R,2S,5S)-3-[2-(1-bicyclo[1.1.1]pentanyl)-2-[(2,2,2-trifluoroacetyl)amino]acetyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (0.45 g, 840.25 umol, 1 eq) was separated by SFC (column: DAICEL CHIRALPAK AD(250 mm*30 mm,10 um); mobile phase: [Neu-IPA]; B %: 20%-20%, 5 min) to give (1R,2S,5S)-3-[2-(1-bicyclo[1.1.1]pentanyl)-2-[(2,2,2-trifluoroacetyl)amino]acetyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide Isomer 1 (170.72 mg, 318.77 umol, 37.94% yield, 100% purity) as a white solid. MS (ESI) m/z 536.2 [M+H]⁺

¹H NMR (400 MHz, DMSO-d₆) δ=9.44 (br s, 1H), 8.83 (d, J=8.2 Hz, 1H), 7.87-7.67 (m, 1H), 5.02-4.86 (m, 1H), 4.73 (br s, 1H), 4.09 (s, 1H), 3.92 (dd, J=5.6, 10.1 Hz, 1H), 3.56-3.47 (m, 1H), 2.72-2.55 (m, 1H), 2.48-2.42 (m, 1H), 2.21-2.09 (m, 1H), 2.05-1.80 (m, 3H), 1.80-1.66 (m, 6H), 1.60 (dd, J=5.7, 7.3 Hz, 1H), 1.39-1.27 (m, 1H), 1.08-1.00 (m, 3H), 1.00-0.79 (m, 3H), 0.75-0.67 (m, 1H), 0.63-0.45 (m, 3H).

(1R,2S,5S)-3-[2-(1-bicyclo[1.1.1]pentanyl)-2-[(2,2,2-trifluoroacetyl)amino]acetyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide Isomer 2 was obtained (160.36 mg, 299.43 umol, 35.64% yield, 100% purity) as a white solid. MS (ESI) m/z 536.2 [M+H]⁺

¹H NMR (400 MHz, DMSO-d₆) δ=9.41 (br s, 1H), 9.05 (d, J=8.5 Hz, 1H), 7.76 (s, 1H), 5.03-4.84 (m, 1H), 4.53 (br s, 1H), 4.18 (s, 1H), 3.83-3.71 (m, 1H), 3.66 (br d, J=10.3 Hz, 1H), 2.58 (br dd, J=4.2, 10.2 Hz, 1H), 2.47-2.44 (m, 1H), 2.24-2.13 (m, 1H), 1.99 (d, J=8.9 Hz, 2H), 1.90 (d, J=9.2 Hz, 3H), 1.86-1.77 (m, 1H), 1.72 (br d, J=9.2 Hz, 3H), 1.58 (dd, J=5.5, 7.3 Hz, 1H), 1.30 (d, J=7.6 Hz, 1H), 1.03 (s, 3H), 0.91 (s, 3H), 0.78 0.69 (m, 1H), 0.64-0.49 (m, 3H).

Example 67: Synthesis of (1R,2S,5S)—N-[(1S)-1-cyano-2-[(3R)-5,5-dimethyl-2-oxo-pyrrolidin-3-yl]ethyl]-6,6-dimethyl-3-[3-[methyl(2,2,2-trifluoroethyl)amino]-2-[(2,2,2-trifluoroacetyl)amino]propanoyl]-3-azabicyclo[3.1.0]hexane-2-carboxamide (Compound 346)

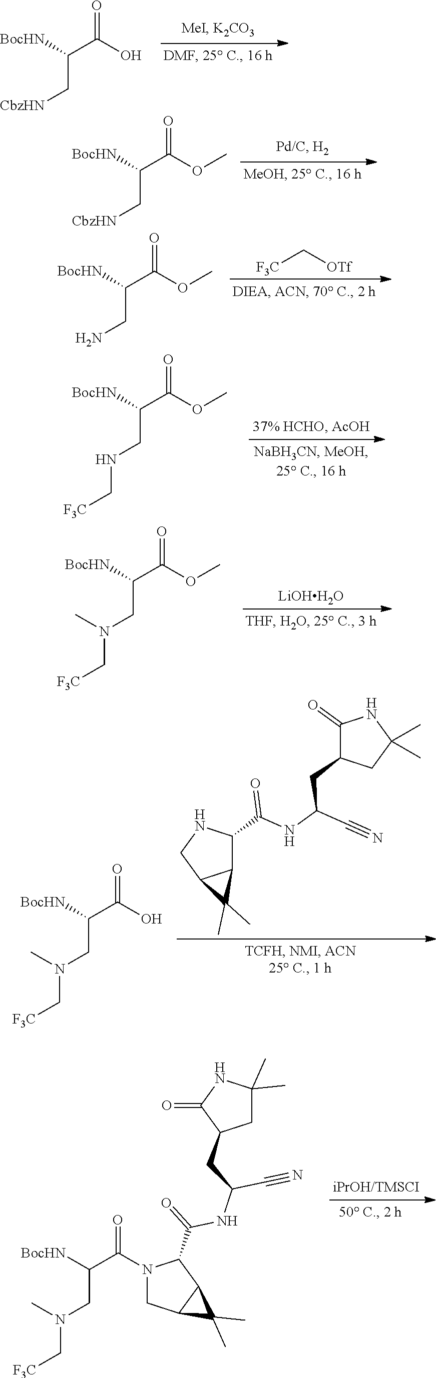

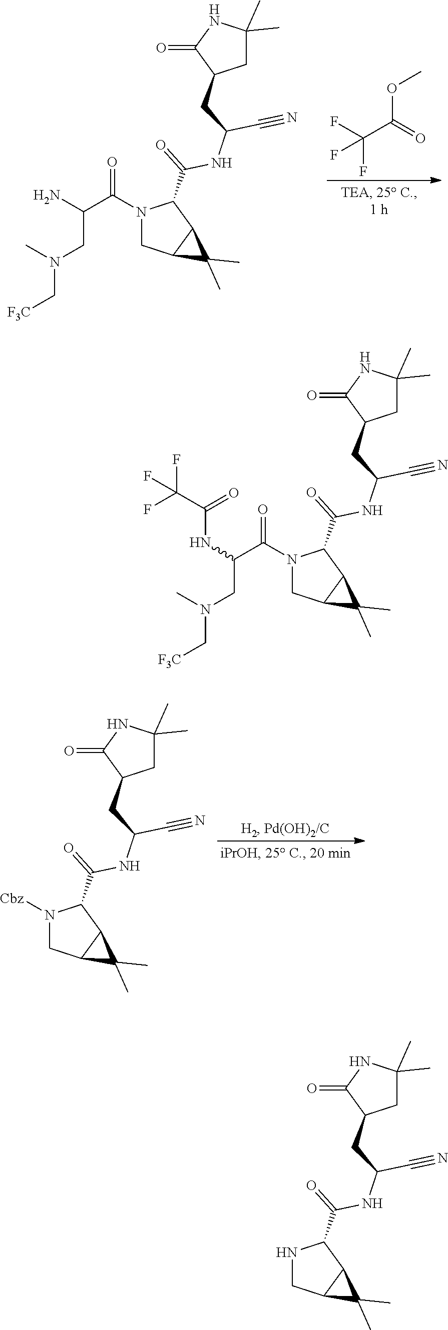

Step 1: methyl 3-(benzyloxycarbonylamino)-2-(tert-butoxycarbonylamino)propanoate

To a solution of 3-(benzyloxycarbonylamino)-2-(tert-butoxycarbonylamino)propanoic acid (10 g, 29.55 mmol, 1 eq) in DMF (100 mL) was added Mel (12.58 g, 88.65 mmol, 5.52 mL, 3 eq) and K₂CO₃ (4.90 g, 35.46 mmol, 1.2 eq). The mixture was stirred at 25° C. for 16 h. The resulting mixture was poured into H₂O (300 mL) and then extracted with ethyl acetate (800 mL*4). The combined organic phase was washed with brine (200 mL), dried over anhydrous Na₂SO₄, filtered and concentrated in vacuo. The residue was purified by column chromatography (SiO₂, Petroleum ether/Ethyl acetate=I/O to 1/1) to give methyl 3-(benzyloxycarbonylamino)-2-(tert-butoxycarbonylamino)propanoate (10 g, 28.38 mmol, 96.04% yield) as a white oil. MS (ESI) m/z 353.2 [M+H]⁺.

Step 2: methyl 3-amino-2-(tert-butoxycarbonylamino)propanoate

To a solution of methyl 3-(benzyloxycarbonylamino)-2-(tert-butoxycarbonylamino)propanoate (10 g, 28.38 mmol, 1 eq) in MeOH (170 mL) was added Pd/C (1 g, 10% purity) under N₂. The suspension was degassed under vacuum and purged with H₂ (57.21 mg, 28.38 mmol, 1 eq) several times. The mixture was stirred under H₂ (57.21 mg, 28.38 mmol, 1 eq) (15 psi) at 25° C. for 16 h. Upon completion, the reaction mixture was filtered and the filtrate was concentrated to give methyl 3-amino-2-(tert-butoxycarbonylamino)propanoate (5.3 g, crude) as a white oil. MS (ESI) m/z 219.1 [M+H]⁺.

Step 3: methyl 2-(tert-butoxycarbonylamino)-3-(2,2,2-trifluoroethylamino)propanoate

To a solution of methyl 3-amino-2-(tert-butoxycarbonylamino)propanoate (5.3 g, 24.28 mmol, 1 eq) in ACN (60 mL) was added DIEA (9.42 g, 72.85 mmol, 12.69 mL, 3 eq) and 2,2,2-trifluoroethyl trifluoromethanesulfonate (11.27 g, 48.57 mmol, 2 eq). The mixture was stirred at 70° C. for 2 h. The resulting reaction mixture was concentrated under reduced pressure to give a residue. The residue was purified by column chromatography (SiO₂, Petroleum ether/Ethyl acetate=I/O to 1/2) to give methyl 2-(tert-butoxycarbonylamino)-3-(2,2,2-trifluoroethylamino)propanoate (3.78 g, 12.59 mmol, 51.84% yield) as a white oil. MS (ESI) m/z 301.2 [M+H]⁺.

Step 4: methyl 2-(tert-butoxycarbonylamino)-3-[methyl(2,2,2-trifluoroethyl)amino]propanoate

To a solution of methyl (2S)-2-(tert-butoxycarbonylamino)-3-(2,2,2-trifluoroethylamino)propanoate (3.7 g, 12.32 mmol, 1 eq) in MeOH (40 mL) was added formaldehyde (34.32 g, 422.90 mmol, 31.49 mL, 37% purity, 34.32 eq), AcOH (1.85 g, 30.81 mmol, 1.76 mL, 2.5 eq) and NaBH3CN (1.55 g, 24.64 mmol, 2 eq). The mixture was stirred at 25° C. for 16 h. Upon completion, the reaction mixture was quenched by addition NaHCO₃aq adjust to pH=9, and extracted with EA (80 mL*3). The combined organic layer was washed with brine (80 mL), dried over Na₂SO₄, filtered and concentrated under reduced pressure to give a residue. The residue was purified by column chromatography (SiO₂, Petroleum ether/Ethyl acetate=I/O to 1/1) to give methyl 2-(tert-butoxycarbonylamino)-3-[methyl(2,2,2-trifluoroethyl)amino]propanoate (3 g, 9.55 mmol, 77.46% yield) as a white oil. MS (ESI) m/z 315.1 [M+H]⁺.

Step 5: 2-(tert-butoxycarbonylamino)-3-[methyl(2,2,2-trifluoroethyl)amino]propanoic acid

To a solution of methyl 2-(tert-butoxycarbonylamino)-3-[methyl(2,2,2-trifluoroethyl)amino]propanoate (3 g, 9.55 mmol, 1 eq) in H₂O (10 mL) and THE (20 mL) was added LiOH.H₂O (1.20 g, 28.64 mmol, 3 eq). The mixture was stirred at 25° C. for 3 h. Upon completion, the reaction mixture was concentrated under reduced pressure to remove THF. The residue was diluted with H₂O 20 mL and extracted with DCM (20 mL*2). The aqueous phase was adjusted pH to around 2 with addition of 0.5 M HCl at 0° C. and then extracted with DCM (20 mL*3). The combined organic phase was washed with brine (20 mL), dried over anhydrous Na₂SO₄, filtered and concentrated in vacuo to give 2-(tert-butoxycarbonylamino)-3-[methyl(2,2,2-trifluoroethyl)amino]propanoic acid (2.3 g, crude) as a yellow oil. MS (ESI) m/z 301.1 [M+H]⁺.

Step 6: (1R,2S,5S)—N-[(1S)-1-cyano-2-[(3R)-5,5-dimethyl-2-oxo pyrrolidin-3-yl]ethyl]-6,6-dimethyl-3-azabicyclo[3.1.0]hexane-2-carboxamide

To a solution of benzyl (1R,2S,5S)-2-[[(1S)-1-cyano-2-[(3R)-5,5-dimethyl-2-oxo-pyrrolidin-3-yl]ethyl]carbamoyl]-6,6-dimethyl-3-azabicyclo[3.1.0]hexane-3-carboxylate (1 g, 2.21 mmol, 1 eq) in i-PrOH (20 mL) was added Pd(OH)₂/C (300 mg, 2.21 mmol, 20% purity, 1 eq) under N₂. The suspension was degassed under vacuum and purged with H₂ (4.46 mg, 2.21 mmol, 1 eq) several times. The mixture was stirred under H₂ (15 psi) at 25° C. for 20 min. Upon completion, the reaction mixture was filtered and the filter was concentrated to give (1R,2S,5S)—N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (0.6 g, crude) was obtained as a white solid. MS (ESI) m/z 319.2 [M+H]⁺.

Step 7: tert-butyl N-[(1S)-2-[(1R,2S,5S)-2-[[(1S)-1-cyan-2-[(3R)-5,5-dimethyl-2-oxo-pyrrolidin-3-yl]ethyl]carbamoyl]-6,6-dimethyl-3-azabicyclo[3.1.0]hexan-3-yl]-1-[[methyl(2,2,2-trifluoroethyl)amino Jmethyl]-2-oxo-ethyl]carbamate

A mixture of (2S)-2-(tert-butoxycarbonylamino)-3-[methyl(2,2,2-trifluoroethyl)amino]propanoic acid (943.03 mg, 1.88 mmol, 60% purity, 1 eq), (1R,2S,5S)—N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (600 mg, 1.88 mmol, 1 eq), NMI (309.41 mg, 3.77 mmol, 300.40 uL, 2 eq), and TCFH (793.06 mg, 2.83 mmol, 1.5 eq) in ACN (15 mL) was stirred at 25° C. for 1 h. Upon completion, the residue was poured into H₂O (50 mL). The aqueous phase was extracted with ethyl acetate (30 mL*3). The combined organic phase was washed with brine (50 mL), dried with anhydrous Na₂SO₄, filtered and concentrated in vacuum. The residue was purified by column chromatography (SiO₂, Petroleum ether/Ethyl acetate=I/O to 2/1) and prep-TLC (SiO₂, PE:EA=1:0) to give tert-butyl N-[(1S)-2 [(1R,2S,5S)-2-[[(1S)-1-cyano-2-[(3R)-5,5-dimethyl-2-oxo-pyrrolidin-3-yl]ethyl]carbamoyl]-6,6-dimethyl-3-azabicyclo[3.1.0]hexan-3-yl]-1-[[methyl(2,2,2-trifluoroethyl)amino]methyl]-2-oxo-ethyl]carbamate (0.44 g, 578.69 umol, 30.71% yield, 79% purity) as a yellow solid. MS (ESI) m/z 601.6 [M+H]⁺.

Step 8: (1R,2S,5S)-3-[(2S)-2-amino-3-[methyl(2,2,2-trifluoroethyl)amino]propanoyl]-N-[(1S)-1-cyano-2-[(3R)-5,5-dimethyl-2-oxo pyrrolidin-3-yl]ethyl]-6,6-dimethyl-3-azabicyclo[3.1.0]hexane-2-carboxamide

A mixture of tert-butyl N-[(1S)-2-[(1R,2S,5S)-2-[[(1S)-1-cyano-2-[(3R)-5,5-dimethyl-2-oxo-pyrrolidin-3-yl]ethyl]carbamoyl]-6,6-dimethyl-3-azabicyclo[3.1.0]hexan-3-yl]-1-[[methyl(2,2,2-trifluoroethyl)amino]methyl]-2-oxo-ethyl]carbamate (430 mg, 715.87 umol, 1 eq) in TMSCI (1 mL) and i-PrOH (3 mL) was stirred at 50° C. for 2 h. Upon completion, the reaction mixture was concentrated under reduced pressure to give (1R,2S,5S)-3-[(2S)-2-amino-3-[methyl(2,2,2-trifluoroethyl)amino]propanoyl]-N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (350 mg, crude) as a yellow gum. MS (ESI) m/z 501.3 [M+H]⁺.

Step 9: (1R,2S,5S)—N-[(1S)-1-cyano-2-[(3R)-5,5-dimethyl-2-oxo pyrrolidin-3-yl]ethyl]-6,6-dimethyl-3-[3-[methyl(2,2,2-trifluoroethyl)amino]-2-[(2,2,2-trifluoroacetyl)amino]propanoyl]-3-azabicyclo[3.1.0]hexane-2-carboxamide

To a solution of (1R,2S,5S)-3-[2-amino-3-[methyl(2,2,2-trifluoroethyl)amino]propanoyl]-N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (400 mg, 799.11 umol, 1 eq) in methyl 2,2,2-trifluoroacetate (5.08 g, 39.68 mmol, 4 mL, 49.65 eq) was added TEA (404.31 mg, 4.00 mmol, 556.13 uL, 5 eq). The mixture was stirred at 25° C. for 1 h. Upon completion, the reaction mixture was concentrated under reduced pressure to give a residue.

The residue was purified by prep-HPLC (neutral condition; column: Phenomenex C18 75*30 mm*3 um; mobile phase: [water(NH₄HCO₃)-ACN]; B %: 30%-60%, 8 min) to give (1R,2S,5S)—N-[(1S)-1-cyano-2-[(3R)-5,5-dimethyl-2-oxo-pyrrolidin-3-yl]ethyl]-6,6-dimethyl-3-[3-[methyl(2,2,2-trifluoroethyl)amino]-2-[(2,2,2-trifluoroacetyl)amino]propanoyl]-3-azabicyclo[3.1.0]hexane-2-carboxamide Isomer 1 (65 mg, 108.63 umol, 13.59% yield, 99.7% purity) as a white solid. MS (ESI) m/z 597.3 [M+H]⁺.

¹H NMR (400 MHz, DMSO-d₆) δ=9.63 (d, J=7.2 Hz, 1H), 8.94 (d, J=8.1 Hz, 1H), 7.85-7.79 (m, 1H), 4.89 (ddd, J=6.2, 8.1, 9.6 Hz, 1H), 4.65-4.53 (m, 1H), 4.12 (s, 1H), 3.87 (dd, J=5.4, 10.3 Hz, 1H), 3.74 (d, J=10.4 Hz, 1H), 3.39-3.32 (m, 1H), 3.27-3.20 (m, 1H), 2.93-2.81 (m, 2H), 2.52 (br s, 1H), 2.44 (s, 3H), 2.19-2.09 (m, 1H), 2.00 (dd, J=8.6, 12.2 Hz, 1H), 1.81-1.68 (m, 1H), 1.60-1.48 (m, 2H), 1.31 (d, J=7.6 Hz, 1H), 1.18 (s, 3H), 1.14-1.09 (m, 3H), 1.05-1.01 (m, 3H), 0.92-0.87 (m, 3H).

¹H NMR (400 MHz, DMSO-d₆+D20) S=8.98 (d, J=8.1 Hz, 1H), 7.84 (s, 1H), 4.94-4.83 (m, 1H), 4.58 (dd, J=6.0, 7.8 Hz, 1H), 4.12 (s, 1H), 3.87 (dd, J=5.4, 10.4 Hz, 1H), 3.73 (d, J=10.5 Hz, 1H), 3.41-3.17 (m, 2H), 2.93-2.83 (m, 2H), 2.57-2.53 (m, 1H), 2.44 (s, 3H), 2.19-2.10 (m, 1H), 2.00 (dd, J=8.5, 12.3 Hz, 1H), 1.74 (ddd, J=6.1, 9.9, 13.5 Hz, 1H), 1.62-1.47 (m, 2H), 1.30 (d, J=7.6 Hz, 1H), 1.20-1.17 (m, 3H), 1.15-1.09 (m, 3H), 1.05-1.01 (m, 3H), 0.92-0.84 (m, 3H).

(1R,2S,5S)—N-[(1S)-1-cyano-2-[(3R)-5,5-dimethyl-2-oxo-pyrrolidin-3-yl]ethyl]-6,6-dimethyl-3-[3-[methyl(2,2,2- trifluoroethyl)amino]-2-[(2,2,2-trifluoroacetyl)amino]propanoyl]-3-azabicyclo[3.1.0]hexane-2-carboxamide Isomer 2 was obtained (63 mg, 105.60 umol, 13.22% yield, 100% purity) as a white solid. MS (ESI) m/z 597.3 [M+H]+.

¹H NMR (400 MHz, DMSO-d₆) δ=9.72 (s, 1H), 9.10-8.97 (m, 1H), 7.99-7.83 (m, 1H), 4.90 (q, J=6.6 Hz, 1H), 4.66-4.60 (m, 1H), 4.13 (s, 1H), 3.91 (br dd, J=5.4, 10.0 Hz, 1H), 3.72 (br d, J=10.4 Hz, 1H), 3.39-3.33 (m, 1H), 3.26-3.21 (m, 1H), 2.90 (br d, J=5.0 Hz, 2H), 2.58 (br d, J=9.5 Hz, 1H), 2.45 (br s, 3H), 2.09 (br dd, J=6.5, 12.8 Hz, 2H), 1.80-1.73 (m, 1H), 1.56-1.49 (m, 2H), 1.37-1.31 (m, 1H), 1.18 (br d, J=11.2 Hz, 6H), 1.02 (s, 3H), 0.90-0.87 (m, 3H).

¹H NMR (400 MHz, DMSO-d₆+D20) δ=9.13-8.93 (m, 1H), 8.00-7.80 (m, 1H), 4.95-4.82 (m, 1H), 4.65-4.53 (m, 1H), 4.11 (s, 1H), 3.96-3.82 (m, 1H), 3.76-3.66 (m, 1H), 3.35-3.18 (m, 2H), 2.94-2.83 (m, 2H), 2.62-2.55 (m, 1H), 2.43 (s, 3H), 2.15 2.03 (m, 2H), 1.77 (td, J=7.2, 14.2 Hz, 1H), 1.57-1.44 (m, 2H), 1.37-1.27 (m, 1H), 1.21 1.11 (m, 6H), 1.00 (s, 3H), 0.91-0.82 (m, 3H).

Example ___: Synthesis of (1R,2S,5S)-N—((S)-1-cyan-2-((R)-5-oxo-4-azaspiro[2.4]heptan-6-yl)ethyl)-6,6-dimethyl-3-((S)-3-(methyl(2,2,2-trifluoroethyl)amino)-2-(2,2,2-trifluoroacetamido)propanoyl)-3-azabicyclo[3.1.0]hexane-2-carboxamide (Compound 347)

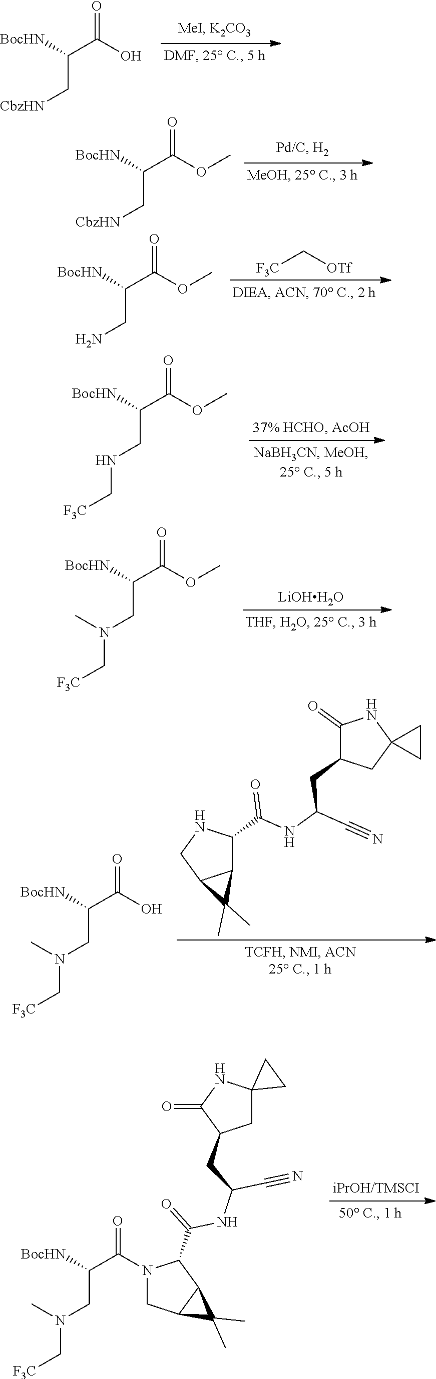

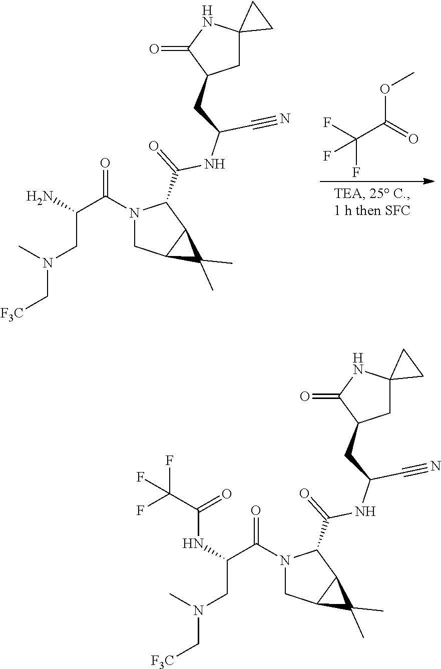

Step 1: (S)-methyl 3-(((benzyloxy)carbonyl)amino)-2-((tert-butoxycarbonyl)amino)propanoate

To a solution of (2S)-3-(benzyloxycarbonylamino)-2-(tert-butoxycarbonylamino) propanoic acid (20 g, 59.11 mmol, 1 eq) in DMF (200 mL) was added K₂CO₃ (9.80 g, 70.93 mmol, 1.2 eq), followed by Mel (25.17 g, 177.33 mmol, 11.04 mL, 3 eq). The mixture was stirred at 25° C. for 5 h. Upon completion, the reaction was poured into H₂O (200 mL), and then extracted with ethyl acetate (120 mL*3). The combined organic phase was washed with brine (80 mL*5), dried over anhydrous Na₂SO₄, filtered and concentrated in vacuum to afford a residue. The residue was purified by column chromatography (SiO₂, Petroleum ether. Ethyl acetate=5:1 to 0:1) to give methyl (2S)-3-(benzyloxycarbonylamino)-2-(tert-butoxycarbonylamino)propanoate (19.6 g, 51.73 mmol, 87.51% yield, 93% purity) as a colorless oil.

Step 2: (S)-methyl 3-amino-2-((tert-butoxycarbonyl)amino)propanoate

To a solution of methyl (2S)-3-(benzyloxycarbonylamino)-2-(tert-butoxycarbonylamino)propanoate (19.6 g, 55.62 mmol, 1 eq) in MeOH (600 mL) was added Pd/C (2.34 g, 10% purity) under N₂. The suspension was degassed under vacuum and purged with H₂ several times. The mixture was stirred under H₂ (15 psi) at 20° C. for 3 hours. Upon completion, the reaction mixture was filtered and the filtrate was concentrated under reduced pressure to give methyl (2S)-3-amino-2-(tert-butoxycarbonylamino)propanoate (11.7 g, crude) as white oil.

Step 3: (S)-methyl 2-((tert-butoxycarbonyl)amino)-3-((2,2,2-trifluoroethyl)amino)propanoate

To a solution of methyl (2S)-3-amino-2-(tert-butoxycarbonylamino)propanoate (11.7 g, 53.61 mmol, 1 eq) in ACN (120 mL) was added DIEA (20.79 g, 160.83 mmol, 28.01 mL, 3 eq) and 2,2,2-trifluoroethyl trifluoromethanesulfonate (24.89 g, 107.22 mmol, 2 eq). The mixture was stirred at 25° C. for 2 h. Upon completion, the reaction mixture was concentrated under reduced pressure to give a residue. The residue was purified by column chromatography (SiO₂, Petroleum ether:Ethyl acetate=20:1 to 1:1) to give methyl (2S)-2-(tert-butoxycarbonylamino)-3-(2,2,2-trifluoroethylamino)propanoate (13.4 g, 44.63 mmol, 83.24% yield) as a colorless oil.

Step 4: (S)-methyl 2-((tert-butoxycarbonyl)amino)-3-(methyl(2,2,2-trifluoroethyl)amino)propanoate

To a solution of methyl (2S)-2-(tert-butoxycarbonylamino)-3-(2,2,2-trifluoroethylamino)propanoate (13.4 g, 44.63 mmol, 1 eq) in MeOH (140 mL) was added formaldehyde (124.29 g, 1.53 mol, 114.03 mL, 37% purity, 34.32 eq), sodium, cyanoboranuide (5.61 g, 89.25 mmol, 2 eq) and AcOH (6.70 g, 111.57 mmol, 6.38 mL, 2.50 eq). The mixture was stirred at 25° C. for 5 h. Upon completion, the reaction mixture was quenched by addition of NaHCO₃aq, adjusted pH to 9, and extracted with ethyl acetate (280 mL*3). The combined organic layer was washed with brine (240 mL), dried over Na₂SO₄, filtered and concentrated under reduced pressure to give a residue. The residue was purified by column chromatography (SiO₂, Petroleum ether:Ethyl acetate=1:0 to 1:1) to give methyl (2S)-2-(tert-butoxycarbonylamino)-3-[methyl(2,2,2-trifluoroethyl)amino]propanoate (13.01 g, 38.37 mmol, 85.99% yield, 92.7% purity) as a colorless oil.

Step 5: (S)-2-((tert-butoxycarbonyl)amino)-3-(methyl(2,2,2-trifluoroethyl)amino)propanoic acid

To a solution of methyl (2S)-2-(tert-butoxycarbonylamino)-3-[methyl(2,2,2-trifluoroethyl)amino]propanoate (2 g, 6.36 mmol, 1 eq) in H₂O (7 mL) and THE (14 mL) was added LiOH.H₂O (801.09 mg, 19.09 mmol, 3 eq). The mixture was stirred at 25° C. for 3 h. Upon completion, the reaction was adjusted pH to 3 at 0° C., and then extracted with DCM (20 mL*3). The combined organic phase was washed with brine (20 mL), dried over anhydrous Na₂SO₄, filtered and concentrated in vacuo to give (2S)-2-(tert-butoxycarbonylamino)-3-[methyl(2,2,2-trifluoroethyl)amino]propanoic acid (1.9 g, crude) as yellow oil.

Step 6: tert-butyl ((S)-1-((1R,2S,5S)-2-(((S)-1-cyano-2-((R)-5-oxo-4-azaspiro(2.4]heptan-6-yl)ethyl)carbamoyl)-6,6-dimethyl-3-azabicyclo[3.1.0]hexan-3-yl)-3-(methyl(2,2,2-trifluoroethyl)amino)-1-oxopropan-2-yl)carbamate

To a solution of (2S)-2-(tert-butoxycarbonylamino)-3-[methyl(2,2,2-trifluoroethyl)amino]propanoic acid (1.71 g, 5.69 mmol, 3 eq) in ACN (25 mL) was added (1R,2S,5S)—N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (600 mg, 1.90 mmol, 1 eq), NMI (311.39 mg, 3.79 mmol, 302.32 uL, 2 eq) and TCFH (798.11 mg, 2.84 mmol, 1.5 eq). The mixture was stirred at 25° C. for 1.5 h. Upon completion, the residue was poured into H₂O (50 mL). The aqueous phase was extracted with EA (30 mL*3). The combined organic phase was washed with brine (50 mL), dried with anhydrous Na₂SO₄, filtered and concentrated in vacuum to afford a residue, which was purified by column chromatography (SiO₂, Petroleum ether:Ethyl acetate =1:0 to 1:1) to give tert-butyl N-[(1S)-2-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexan-3-yl]-1-[[methyl(2,2,2-trifluoroethyl)amino]methyl]-2-oxo-ethyl]carbamate (2.1 g, 1.47 mmol, 77.32% yield, 41.8% purity) as a yellow solid.

Step 7: (1R,2S,5S)-3-((S)-2-amino-3-(methyl(2,2,2-trifluoroethyl)amino)propanoyl)-N—((S)-1-cyano-2-((R)-5-oxo-4-azaspiro[2.4]heptan-6-yl)ethyl)-6,6-dimethyl-3-azabicyclo[3.1.0]hexane-2-carboxamide

A mixture of tert-butyl N-[(1S)-2-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexan-3-yl]-1-[[methyl(2,2,2-trifluoroethyl)amino]methyl]-2-oxo-ethyl]carbamate (2 g, 3.34 mmol, 1 eq), TMSCI (6.85 g, 63.03 mmol, 8.00 mL, 18.87 eq) and i-PrOH (15.70 g, 261.25 mmol, 20.00 mL, 78.20 eq) was degassed and purged with N₂ for 3 times, and then the mixture was stirred at 50° C. for 1 h under N₂ atmosphere. Upon completion, the reaction mixture was concentrated under reduced pressure to give (1R,2S,5S)-3-[(2S)-2-amino-3-[methyl(2,2,2-trifluoroethyl)amino]propanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (800 mg, crude) as a yellow solid.

Step 8: (1R,2S,5S)-N—((S)-1-cyan-2-((R)-5-oxo-4-azaspiro[2.4]heptan-6-yl)ethyl)-6,6-dimethyl-3-((S)-3-(methyl(2,2,2-trifluoroethyl)amino)-2-(2,2,2-trifluoroacetamido)propanoyl)-3-azabicyclo[3.1.0]hexane-2-carboxamide

To a solution of (1R,2S,5S)-3-[(2S)-2-amino-3-[methyl(2,2,2-trifluoroethyl)amino]propanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (800 mg, 1.60 mmol, 1 eq) in TEA (974.26 mg, 9.63 mmol, 1.34 mL, 6 eq) was added methyl 2,2,2-trifluoroacetate (10.20 g, 79.67 mmol, 8.03 mL, 49.65 eq). The mixture was stirred at 25° C. for 1 h. Upon completion, the reaction mixture was concentrated under reduced pressure to give a residue. The residue was purified by prep-HPLC (column: Phenomenex luna C18 (250*70 mm, 15 um); mobile phase: [water(FA)-ACN]; B %: 40%-80%, 20 min), which was further separated by SFC (column: ChiralPak 111, 250*30 mm, 10 um; mobile phase: [Neu-ETOH]; B %: 40% 40%, 10 min) to give (1R,2S,5S)—N-[(1S)-1-cyano-2-[(6R)-5-oxo-4-azaspiro[2.4]heptan-6-yl]ethyl]-6,6-dimethyl-3-[(2S)-3-[methyl(2,2,2-trifluoroethyl)amino]-2-[(2,2,2-trifluoroacetyl)amino]propanoyl]-3-azabicyclo[3.1.0]hexane-2-carboxamide Isomer 1 (65.81 mg, 110.69 umol, 6.90% yield, 100% purity) as a white solid. MS (ESI) m/z 595.2[M+H]⁺.

¹H NMR (400 MHz, DMSO-d₆) δ=9.73-9.64 (m, 1H), 9.04-8.45 (m, 1H), 7.08-7.09 (m, 1H), 5.03-4.84 (m, 1H), 4.70-4.55 (m, 1H), 4.18-4.11 (m, 1H), 3.94-3.84 (m, 1H), 3.79-3.70 (m, 1H), 3.33-3.18 (m, 2H), 2.98-2.84 (m, 2H), 2.69-2.59 (m, 1H), 2.47-2.42 (m, 3H), 1.98 (br t, J=11.1 Hz, 3H), 1.90-1.78 (m, 1H), 1.61-1.53 (m, 1H), 1.38-1.30 (m, 1H), 1.06-1.00 (m, 3H), 0.91-0.88 (m, 3H), 0.81-0.72 (m, 1H), 0.69-0.52 (m, 3H)

(1R,2S,5S)—N-[(1S)-1-cyano-2-[(6R)-5-oxo-4-azaspiro[2.4]heptan-6-yl]ethyl]-6,6-dimethyl-3-[(2S)-3-[methyl(2,2,2-trifluoroethyl)amino]-2-[(2,2,2-trifluoroacetyl)amino]propanoyl]-3-azabicyclo[3.1.0]hexane-2-carboxamide Isomer 2 was obtained (74.46 mg, 125.24 umol, 7.80% yield, 100% purity) as a white solid. MS (ESI) m/z 595.2[M+H]⁺.

¹H NMR (400 MHz, DMSO-d₆) δ=9.68 (br d, J=7.0 Hz, 1H), 8.97 (d, J=8.1 Hz, 1H), 7.78 (s, 1H), 4.98-4.84 (m, 1H), 4.59 (q, J=7.1 Hz, 1H), 4.13 (s, 1H), 3.92-3.83 (m, 1H), 3.75 (d, J=10.4 Hz, 1H), 3.46 (s, 1H), 3.30-3.20 (m, 1H), 2.92-2.83 (m, 2H), 2.68-2.53 (m, 1H), 2.45 (s, 3H), 2.24-2.11 (m, 1H), 1.96 (d, J=9.0 Hz, 2H), 1.83 (ddd, J=6.3, 9.9, 13.5 Hz, 1H), 1.58 (dd, J=5.4, 7.4 Hz, 1H), 1.32 (d, J=7.6 Hz, 1H), 1.06-1.00 (m, 3H), 0.90 (s, 3H), 0.77-0.72 (m, 1H), 0.62-0.52 (m, 3H)

Example 68: Synthesis of (1R,2S,5S)-N—((S)-1-cyan-2-((R)-5,5-dimethyl-2-oxopyrrolidin-3-yl)ethyl)-6,6-dimethyl-3-(2-(2,2,2-trifluoroacetamido)-2-(1-(trifluoromethyl)cyclopropyl)acetyl)-3-azabicyclo[3.1.0]hexane-2-carboxamide (Compound 351)

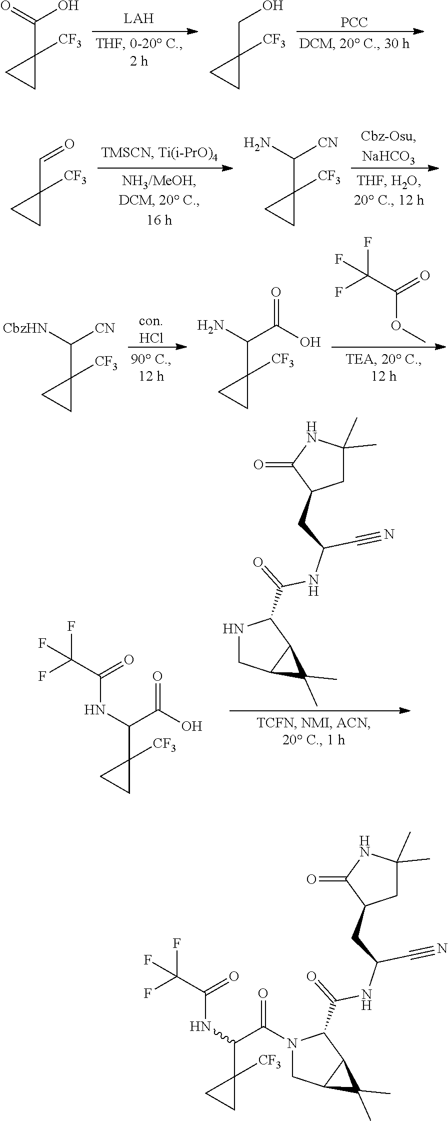

Step 1: (1-(trifluoromethyl)cyclopropyl)methanol

A solution of 1-(trifluoromethyl)cyclopropanecarboxylic acid (10 g, 64.90 mmol, 1 eq) in THE (150 mL) was added LAH (2.96 g, 77.88 mmol, 1.2 eq) in THE (10 mL) dropwise at 0° C. under N₂ atmosphere. The mixture was stirred at 20° C. for 2 h. Upon completion, the reaction mixture was quenched by addition H₂O (8 mL) at 0° C., and the added 15% NaOH solution (8 mL) and added H₂O (4 mL). The mixture was stirred for 20 min and filtered and the solution was concentrated under reduced pressure to give [1-(trifluoromethyl)cyclopropyl]methanol (9 g, crude) as a colorless oil.

Step 2: 1-(trifluoromethyl)cyclopropanecarbaldehyde

A solution of [1-(trifluoromethyl)cyclopropyl]methanol (9 g, 57.81 mmol, 10.15 mL, 90% purity, 1 eq) in DCM (800 mL) was added with PCC (18.69 g, 86.72 mmol, 1.5 eq), and then the mixture was stirred at 20° C. for 30 h. Upon completion, the reaction mixture was filtered to give 1-(trifluoromethyl)cyclopropanecarbaldehyde (7.98 g, crude) as a black brown liquid and used next step directly.

Step 3: 2-amino-2-(1-(trifluoromethyl)cyclopropyl)acetonitrile

A solution of 1-(trifluoromethyl)cyclopropanecarbaldehyde (7.89 g, 57.14 mmol, 1 eq), Ti(i-PrO)₄ (24.36 g, 85.71 mmol, 25.29 mL, 1.5 eq) and NH₃/MeOH (7 M, 32.65 mL, 4 eq) in DCM (200 mL) was stirred for 1 h, then TMSCN (17.01 g, 171.41 mmol, 21.44 mL, 3 eq) was added. The mixture was stirred at 20° C. for 15 h. Upon completion, the reaction mixture was diluted H₂O (200 mL), and then extracted with DCM (150 mL*3). The combined organic layers were washed with brine (200 mL), dried over Na₂SO₄, filtered and concentrated under reduced pressure to give a residue. The residue was purified by column chromatography (SiO₂, Petroleum ether:Ethyl acetate=10:1 to 0:1) to give 2-amino-2-[1-(trifluoromethyl)cyclopropyl]acetonitrile (6 g, 36.56 mmol, 63.98% yield) as a yellow oil.

Step 4: benzyl (cyano(1-(trifluoromethyl)cyclopropyl)methyl)carbamate

To a solution of benzyl (2,5-dioxopyrrolidin-1-yl) carbonate (13.67 g, 54.84 mmol, 1.5 eq) in THE (60 mL) was added 2-amino-2-[1-(trifluoromethyl)cyclopropyl]acetonitrile (6 g, 36.56 mmol, 1 eq), and then NaHCO₃(9.21 g, 109.67 mmol, 4.27 mL, 3 eq) in H₂O (120 mL) was added. The mixture was stirred at 20° C. for 12 h, and then the reaction mixture was extracted with EA (50 mL*3). The combined organic layers were washed with brine (50 mL*1), dried over Na₂SO₄, filtered and concentrated under reduced pressure to give a residue. The residue was purified by column chromatography (SiO₂, Petroleum ether:Ethyl acetate=10:1 to 1:1) to give benzyl N-[cyano-[1-(trifluoromethyl)cyclopropyl]methyl]carbamate (9.5 g, 31.85 mmol, 87.13% yield) as a white solid.

Step 5: 2-amino-2-(1-(trifluoromethyl)cyclopropyl)acetic acid

A solution of benzyl N-[cyano-[1-(trifluoromethyl)cyclopropyl]methyl]carbamate (2 g, 6.71 mmol, 1 eq) in HCl (12 M, 20 mL, 35.79 eq) was stirred at 90° C. for 12 h. Upon completion, the reaction mixture was concentrated under reduced pressure to give 2-amino-2-[1-(trifluoromethyl)cyclopropyl]acetic acid (520 mg, crude) as a white solid. MS (ESI) m/z 184.2 [M−H]⁺.

Step 6: 2-(2,2,2-trifluoroacetamido)-2-(1-(trifluoromethyl)cyclopropyl)acetic acid

A solution of 2-amino-2-[1-(trifluoromethyl)cyclopropyl]acetic acid (520 mg, 2.84 mmol, 1 eq) in methyl 2,2,2-trifluoroacetate (10.34 g, 80.78 mmol, 8.15 mL, 28.45 eq) was added TEA (862.00 mg, 8.52 mmol, 1.19 mL, 3 eq) and stirred at 20° C. for 12 h. Upon completion, the reaction mixture was concentrated under reduced pressure to remove solvent. The residue was diluted with H₂O (10 mL) and adjusted to pH=3 with 1 M HCl and extracted with EA (10 mL*3). The combined organic layers were washed with brine (10 mL*1), dried over Na₂SO₄, filtered and concentrated under reduced pressure to give 2-[(2,2,2-trifluoroacetyl)amino]-2-[1-(trifluoromethyl)cyclopropyl]acetic acid (750 mg, crude) as a yellow oil. MS (ESI) m/z 278.1 [M−H]⁺.

Step 7: (1R,2S,5S)-N—((S)-1-cyan-2-((R)-5,5-dimethyl-2-oxopyrrolidin-3-yl)ethyl)-6,6-dimethyl-3-(2-(2,2,2-trifluoroacetamido)-2-(1-(trifluoromethyl)cyclopropyl)acetyl)-3-azabicyclo[3.1.0]hexane-2-carboxamide

A solution of 2-[(2,2,2-trifluoroacetyl)amino]-2-[1-(trifluoromethyl)cyclopropyl]acetic acid (500 mg, 1.79 mmol, 1 eq) in ACN (10 mL) was added (1R,2S,5S)—N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (627.39 mg, 1.97 mmol, 1.1 eq) and 1-methylimidazole (588.24 mg, 7.16 mmol, 571.11 uL, 4 eq). Then TCFH (603.10 mg, 2.15 mmol, 1.2 eq) was added to the mixture and the mixture was sitrred at 20° C. for 1 h. Upon completion, the reaction mixture was quenched by addition H₂O 15 mL at 0° C., and extracted with DCM (15 mL*3). The combined organic layers were washed with brine (15 mL), dried over Na₂SO₄, filtered and concentrated under reduced pressure to give a residue. The residue was purified by prep-HPLC (column: Waters Xbridge BEH C18 250*70 mm*10 um; mobile phase: [water (NH₄HCO₃)-ACN]; B %: 35%-65%, 20 min) to give (1R,2S,5S)—N-[(1S)-1-cyano-2-[(3R)-5,5-dimethyl-2-oxo-pyrrolidin-3-yl]ethyl]-6,6-dimethyl-3-[2-[(2,2,2-trifluoroacetyl)amino]-2-[1-(trifluoromethyl)cyclopropyl]acetyl]-3-azabicyclo[3.1.0]hexane-2-carboxamide Isomer 1 (64.81 mg, 111.83 umol, 6.24% yield, 100% purity) as white solid. MS (ESI) m/z 580.3 [M+H]⁺.

¹H NMR (400 MHz, CD₃CN-d₃) δ=8.52 (br d, J=5.1 Hz, 1H), 7.83 (br d, J=7.0 Hz, 1H), 6.33 (br s, 1H), 5.17-5.03 (m, 1H), 4.77 (td, J=6.5, 10.1 Hz, 1H), 4.61-4.09 (m, 2H), 3.62-3.53 (m, 1H), 2.69-2.60 (m, 1H), 2.17-2.10 (m, 2H), 1.92-1.86 (m, 1H), 1.69-1.43 (m, 4H), 1.27-1.24 (m, 3H), 1.21-1.19 (m, 3H), 1.17-1.12 (m, 2H), 1.06-1.03 (m, 3H), 0.98-0.84 (m, 3H), 0.83-0.76 (m, 1H)

(1R,2S,5S)—N-[(1S)-1-cyano-2-[(3R)-5,5-dimethyl-2-oxo-pyrrolidin-3-yl]ethyl]-6,6-dimethyl-3-[2-[(2,2,2-trifluoroacetyl)amino]-2-[1-(trifluoromethyl)cyclopropyl]acetyl]-3-azabicyclo[3.1.0]hexane-2-carboxamide Isomer 2 was obtained (71.19 mg, 117.68 umol, 6.57% yield, 95.8% purity) as a white solid. MS (ESI) m/z 580.3 [M+H]⁺.

¹H NMR (400 MHz, CD₃CN-d₃) δ=7.98 (br d, J=6.9 Hz, 1H), 7.91 (br d, J=5.9 Hz, 1H), 6.34 (br s, 1H), 5.19 (br d, J=6.7 Hz, 1H), 4.83 (td, J=6.4, 10.2 Hz, 1H), 4.05 (s, 1H), 3.87 (dd, J=5.5, 10.3 Hz, 1H), 3.78-3.72 (m, 1H), 2.68-2.57 (m, 1H), 2.26-2.17 (m, 1H), 2.14-2.09 (m, 1H), 1.92-1.85 (m, 1H), 1.65-1.58 (m, 3H), 1.41 (d, J=7.5 Hz, 1H), 1.26 (s, 3H), 1.22 (s, 3H), 1.11-1.06 (m, 3H), 1.06 (s, 3H), 0.96 (s, 3H)

Example ___: Synthesis of (1R,2S,5S)—N-[(1S)-1-cyano-2-[(6R)-5-oxo-4-azaspiro[2.4]heptan-6-yl]ethyl]-6,6-dimethyl-3-[2-[(2,2,2-trifluoroacetyl)amino]-2-[1-(trifluoromethyl)cyclopropyl]acetyl]-3-azabicyclo[3.1.0]hexane-2-carboxamide (Compound 352)

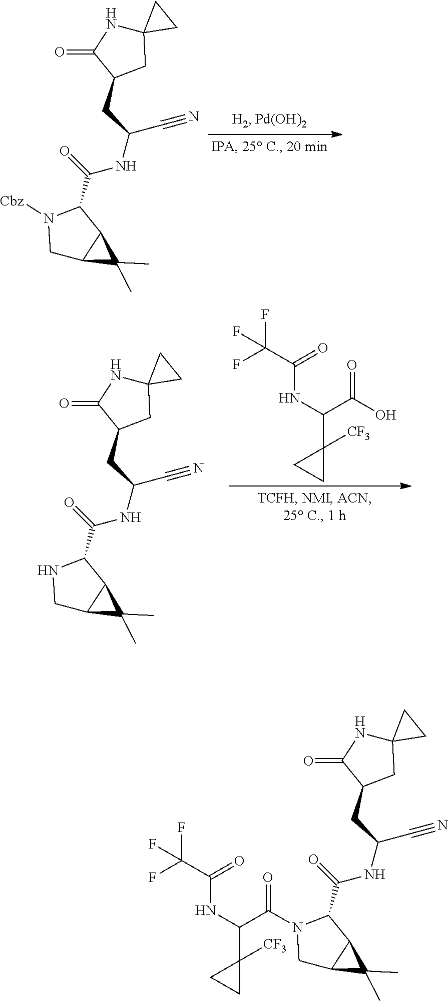 [SMILES: CC1(C)[C@@H]2[C@@H](C(=O)N[C@H](C#N)C[C@@H]3CC4(CC4)NC3=O)N(C(=O)OCc3ccccc3)C[C@@H]21 ; CC1(C)[C@@H]2[C@@H](C(=O)N[C@H](C#N)C[C@@H]3CC4(CC4)NC3=O)NC[C@@H]21 ; O=C(O)C(NC(=O)C(F)(F)F)C1(C(F)(F)F)CC1 ; CC1(C)[C@@H]2[C@@H](C(=O)N[C@H](C#N)C[C@@H]3CC4(CC4)NC3=O)N(C(=O)C(NC(=O)C(F)(F)F)C3(C(F)(F)F)CC3)C[C@@H]21]

Step 1: (1R,2S,5S)—N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide

To a solution of benzyl (1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carboxylate (1.5 g, 3.33 mmol, 1 eq) in IPA (40 mL) was added Pd(OH)₂ (750.00 mg, 1.07 mmol, 20% purity, 3.21e-1 eq) and the resulting mixture was stirred under H₂ atmosphere at 25° C. for 20 min. Upon completion, the reaction mixture was filtered and concentrated under reduced pressure to give (1R,2S,5S)—N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (1.2 g, crude) as a white solid. MS (ESI) m/z 317.3 [M+H]⁺.

Step 2: (1R,2S,5S)—N-[(1S)-1-cyano-2-[(6R)-5-oxo-4-azaspiro[2.4]heptan-6-yl]ethyl]-6,6-dimethyl-3-[2-[(2,2,2-trifluoroacetyl)amino]-2-[1-(trifluoromethyl)cyclopropyl]acetyl]-3-azabicyclo[3.1.0]hexane-2-carboxamide

A solution of 2-[(2,2,2-trifluoroacetyl)amino]-2-[1-(trifluoromethyl)cyclopropyl]acetic acid (529.34 mg, 1.90 mmol, 1 eq) and (1R,2S,5S)—N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (600 mg, 1.90 mmol, 1 eq) in ACN (15 mL) was added with 1-methylimidazole (622.79 mg, 7.59 mmol, 604.65 uL, 4 eq). Then, TCFH (638.49 mg, 2.28 mmol, 1.2 eq) was added at 0° C. and the mixture was stirred at 25° C. for 1 h. Upon completion, the reaction mixture was diluted with H₂O (20 mL) and extracted with DCM (15 mL*3). The combined organic layers were washed with brine (30 mL), dried over Na₂SO₄, filtered and concentrated under reduced pressure to give a residue. The residue was purified by prep-HPLC (column: Waters Xbridge BEH C18 250*50 mm*10 um; mobile phase: [water(NH₄HCO₃)⁻ACN]; B %: 35%-55%, 10 min) to give (1R,2S,5S)—N-[(1S)-1-cyano-2-[(6R)-5-oxo-4-azaspiro[2.4]heptan-6-yl]ethyl]-6,6-dimethyl-3-[2-[(2,2,2-trifluoroacetyl)amino]-2-[1-(trifluoromethyl)cyclopropyl]acetyl]-3-azabicyclo[3.1.0]hexane-2-carboxamide Isomer 1 (64.63 mg, 102.06 umol, 5.38% yield, 91.2% purity) as a light yellow solid. MS (ESI) m/z 578.3 [M+H]⁺.

¹H NMR (400 MHz, DMSO-d₆) δ=10.39-9.84 (m, 1H), 9.36-8.73 (m, 1H), 7.90-7.70 (m, 1H), 5.41-4.97 (m, 1H), 4.97-4.84 (m, 1H), 4.64-4.05 (m, 1H), 4.04-3.68 (m, 1H), 3.55-3.47 (m, 1H), 2.64-2.53 (m, 1H), 2.19-1.98 (m, 2H), 1.98-1.63 (m, 3H), 1.48-1.24 (m, 2H), 0.80 (s, 9H), 0.75-0.69 (m, 1H), 0.69-0.59 (m, 1H), 0.58-0.53 (m, 2H)

(1R,2S,5S)—N-[(1S)-1-cyano-2-[(6R)-5-oxo-4-azaspiro[2.4]heptan-6-yl]ethyl]-6,6-dimethyl-3-[2-[(2,2,2-trifluoroacetyl)amino]-2-[1-(trifluoromethyl)cyclopropyl]acetyl]-3-azabicyclo[3.1.0]hexane-2-carboxamide Isomer 2 was obtained (70.48 mg, 117.77 umol, 6.21% yield, 96.5% purity) as a light yellow solid. MS (ESI) m/z 578.3 [M+H]⁺.

¹H NMR (400 MHz, DMSO-d₆) S=10.14 (br d, J=4.2 Hz, 1H), 9.16 (d, J=8.4 Hz, 1H), 7.76 (s, 1H), 5.12 (br s, 1H), 4.94 (dt, J=5.2, 9.5 Hz, 1H), 4.10 (s, 1H), 3.81-3.65 (m, 2H), 2.64-2.53 (m, 1H), 2.21-2.11 (m, 1H), 1.96 (dq, J=9.0, 12.5 Hz, 2H), 1.81 (ddd, J=5.4, 10.7, 13.5 Hz, 1H), 1.66 (dd, J=5.2, 7.4 Hz, 1H), 1.50-1.42 (m, 1H), 1.36 (d, J=7.6 Hz, 1H), 1.18-1.11 (m, 1H), 1.05 (s, 3H), 1.03-0.96 (m, 2H), 0.93 (s, 3H), 0.78-0.71 (m, 1H), 0.65-0.60 (m, 1H), 0.59-0.51 (m, 2H)

Example ___: Synthesis of (1R,2S,5S)-N—((S)-1-cyano-2-((R)-5,5-dimethyl-2-oxopyrrolidin-3-yl)ethyl)-6,6-dimethyl-3-(2-(2,2,2-trifluoroacetamido)-2-(1-(trifluoromethyl)cyclobutyl)acetyl)-3-azabicyclo[3.1.0]hexane-2-carboxamide (Compound 353)

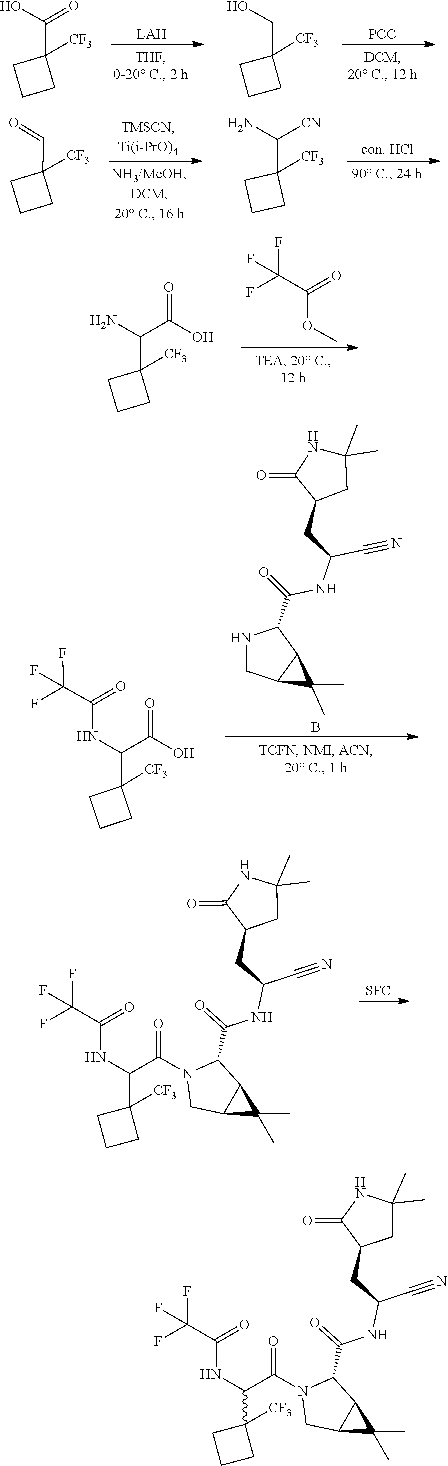

A solution of 1-(trifluoromethyl)cyclobutanecarboxylic acid (5 g, 29.74 mmol, 1 eq) in THF (50 mL) was added LAH (1.35 g, 35.69 mmol, 1.2 eq) in THE (50 mL) dropwise at 0° C. under N₂ atmosphere. The mixture was stirred at 20° C. for 2 h. Upon completion, the reaction mixture was quenched by addition H₂O (1.5 mL) at 0° C., and the added 15% NaOH solution (4.5 mL) and added H₂O (1.5 mL). The mixture was stirred for 20 min, and then filtered. The solution was concentrated under reduced pressure to give [1-(trifluoromethyl)cyclobutyl]methanol (4 g, crude) as a colorless oil.

Step 2: 1-(trifluoromethyl)cyclobutanecarbaldehyde

A solution of [1-(trifluoromethyl)cyclobutyl]methanol (4 g, 23.36 mmol, 10.15 mL, 90% purity, 1 eq) in DCM (800 mL) was added PCC (10.07 g, 46.71 mmol, 2 eq), and then the mixture was stirred at 20° C. for 12 h. Upon completion, the reaction mixture was filtered to give 1-(trifluoromethyl)cyclobutanecarbaldehyde (3.55 g, crude) as a black brown liquid and used next step directly.

Step 3: 2-amino-2-(1-(trifluoromethyl)cyclobutyl)acetonitrile

A solution of 1-(trifluoromethyl)cyclobutanecarbaldehyde (3.55 g, 23.34 mmol, 1 eq), Ti(i-PrO)₄ (9.95 g, 35.01 mmol, 10.33 mL, 1.5 eq) and NH₃/MeOH (7 M, 13.34 mL, 4 eq) in DCM (50 mL) was stirred for 1 h, and then TMSCN (6.95 g, 70.01 mmol, 8.76 mL, 3 eq) was added. The mixture was stirred at 20° C. for 15 h. Upon completion, the reaction mixture was diluted with H₂O (100 mL), filtered and then extracted with DCM (100 mL*3). The combined organic layers were washed with brine (100 mL), dried over Na₂SO₄, filtered and concentrated under reduced pressure to give a residue. The residue was purified by column chromatography (SiO₂, Petroleum ether:Ethyl acetate=10:1 to 0:1) to give 2-amino-2-[1-(trifluoromethyl)cyclobutyl]acetonitrile (2 g, 11.23 mmol, 48.10% yield) as a yellow oil.

Step 4: 2-amino-2-(1-(trifluoromethyl)cyclobutyl)acetic acid

A solution of 2-amino-2-[1-(trifluoromethyl)cyclobutyl]acetonitrile (2 g, 11.23 mmol, 1 eq) in HCl (12 M, 20.00 mL, 21.38 eq) was stirred at 90° C. for 24 h. Upon completion, the reaction mixture was concentrated under reduced pressure to give 2-amino-2-[1-(trifluoromethyl)cyclobutyl]acetic acid (2.2 g, crude) as a white solid. MS (ESI) m/z 198.2 [M+H]⁺.

Step 5: 2-(2,2,2-trifluoroacetamido)-2-(1-(trifluoromethyl)cyclobutyl)acetic acid

A solution of 2-amino-2-[1-(trifluoromethyl)cyclobutyl]acetic acid (2.2 g, 11.16 mmol, 1 eq) in methyl 2,2,2-trifluoroacetate (40.65 g, 317.47 mmol, 32.01 mL, 28.45 eq) was added with TEA (3.39 g, 33.48 mmol, 4.66 mL, 3 eq), and the resulting mixture was stirred at 20° C. for 12 h. Upon completion, the reaction mixture was concentrated under reduced pressure to remove solvent. The residue was diluted with H₂O (30 mL) and adjusted to pH=3 with 1 M HCl and extracted with EA (30 mL*3). The combined organic layers were washed with brine (30 mL), dried over Na₂SO₄, filtered and concentrated under reduced pressure to give 2-[(2,2,2-trifluoroacetyl)amino]-2-[1-(trifluoromethyl)cyclobutyl]acetic acid (2.5 g, crude) as a yellow oil. MS (ESI) m/z 294.1 [M+H]⁺.

Step 6: (1R,2S,5S)-N—((S)-1-cyan-2-((R)-5,5-dimethyl-2-oxopyrrolidin-3-yl)ethyl)-6,6-dimethyl-3-(2-(2,2,2-trifluoroacetamido)-2-(1-(trifluoromethyl)cyclobutyl)acetyl)-3-azabicyclo[3.1.0]hexane-2-carboxamide

A solution of (1R,2S,5S)—N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (350 mg, 1.10 mmol, 1 eq) in ACN (10 mL) was added 2-[(2,2,2-trifluoroacetyl)amino]-2-[1-(trifluoromethyl)cyclobutyl]acetic acid (500 mg, 1.71 mmol, 1.55 eq) and 1-methylimidazole (360.98 mg, 4.40 mmol, 350.46 uL, 4 eq). TCFH (370.10 mg, 1.32 mmol, 1.2 eq) was then added to the mixture and the mixture was stirred at 20° C. for 1 h. Upon completion, the reaction mixture was quenched by addition H₂O (10 mL) at 0° C., and extracted with DCM (10 mL*3). The combined organic layers were washed with brine (10 mL), dried over Na₂SO₄, filtered and concentrated under reduced pressure to give a residue. The residue was purified by prep-HPLC (column: Waters Xbridge BEH C18 250*70 mm*10 um; mobile phase: [water (NH₄HCO₃)-ACN]; B %: 40%-70%, 20 min) to give (1R,2S,5S)—N-[(1S)-1-cyano-2-[(3R)-5,5-dimethyl-2-oxo-pyrrolidin-3-yl]ethyl]-6,6-dimethyl-3-[2-[(2,2,2-trifluoroacetyl)amino]-2-[1-(trifluoromethyl)cyclobutyl]acetyl]-3-azabicyclo[3.1.0]hexane-2-carboxamide (300 mg, 485.21 umol, 44.14% yield, 96% purity) as yellow solid. MS (ESI) m/z 594.3 [M+H]⁺.

Step 7: (1R,2S,5S)-N—((S)-1-cyan-2-((R)-5,5-dimethyl-2-oxopyrrolidin-3-yl)ethyl)-6,6-dimethyl-3-(2-(2,2,2-trifluoroacetamido)-2-(1-(trifluoromethyl)cyclobutyl)acetyl)-3-azabicyclo[3.1.0]hexane-2-carboxamide

(1R,2S,5S)—N-[(1S)-1-cyano-2-[(3R)-5,5-dimethyl-2-oxo-pyrrolidin-3-yl]ethyl]-6,6-dimethyl-3-[2-[(2,2,2-trifluoroacetyl)amino]-2-[1-(trifluoromethyl)cyclobutyl]acetyl]-3-azabicyclo[3.1.0]hexane-2-carboxamide (300 mg, 505.42 umol, 1.10e-2 eq) was separated by SFC (column: REGIS(S,S)WHELK-01(250 mm*25 mm, 10 um); mobile phase: [Neu-IPA]; B %: 20%-20%, 7 min) to give (1R,2S,5S)—N-[(1S)-1-cyano-2-[(3R)-5,5-dimethyl-2-oxo-pyrrolidin-3-yl]ethyl]-6,6-dimethyl-3-[2-[(2,2,2-trifluoroacetyl)amino]-2-[1-(trifluoromethyl)cyclobutyl]acetyl]-3-azabicyclo[3.1.0]hexane-2-carboxamide Isomer 1 (73.21 mg, 123.34 umol, 24.40% yield, 100% purity) as white solid. MS (ESI) m/z 594.3 [M+H]⁺.

¹H NMR (400 MHz, DMSO-d₆) δ=10.07 (br s, 1H), 9.19-9.02 (m, 1H), 7.83 (s, 1H), 4.98-4.81 (m, 2H), 4.20 (s, 1H), 3.84-3.57 (m, 2H), 2.83-2.70 (m, 1H), 2.67-2.53 (m, 2H), 2.17 (dt, J=4.9, 9.3 Hz, 2H), 2.01 (br dd, J=8.8, 12.4 Hz, 2H), 1.92-1.68 (m, 3H), 1.65-1.46 (m, 2H), 1.35 (d, J=7.6 Hz, 1H), 1.22-1.15 (m, 3H), 1.11 (s, 3H), 1.04 (s, 3H), 0.86 (s, 3H)

(1R,2S,5S)—N-[(1S)-1-cyano-2-[(3R)-5,5-dimethyl-2-oxo-pyrrolidin-3-yl]ethyl]-6,6-dimethyl-3-[2-[(2,2,2-trifluoroacetyl)amino]-2-[1-(trifluoromethyl)cyclobutyl]acetyl]-3-azabicyclo[3.1.0]hexane-2-carboxamide Isomer 2 was obtained (68.27 mg, 115.02 umol, 22.76% yield, 100% purity) as white solid. MS (ESI) m/z 594.3 [M+H]⁺.

¹H NMR (400 MHz, DMSO-d₆) δ=10.29 (br d, J=0.9 Hz, 1H), 9.28-8.94 (m, 1H), 7.87 (br d, J=13.9 Hz, 1H), 5.02-4.60 (m, 2H), 4.36-4.08 (m, 1H), 3.90-3.58 (m, 1H), 3.58-3.42 (m, 1H), 2.85-2.68 (m, 1H), 2.63-2.56 (m, 1H), 2.19 (br d, J=5.0 Hz, 1H), 2.18-1.92 (m, 4H), 1.92-1.71 (m, 3H), 1.64-1.46 (m, 2H), 1.42-1.29 (m, 1H), 1.22 1.17 (m, 3H), 1.16-1.08 (m, 3H), 1.03 (d, J=3.9 Hz, 3H), 0.93-0.79 (m, 3H)

Example 69: Synthesis of (1R,2S,5S)—N-[(1S)-1-cyan-2-[(6R)-5-oxo-4-azaspiro[2.4]heptan-6-yl]ethyl]-6,6-dimethyl-3-[2-[(2,2,2-trifluoroacetyl)amino]-2-[1-(trifluoromethyl)cyclobutyl]acetyl]-3-azabicyclo[3.1.0]hexane-2-carboxamide (Compound 354)

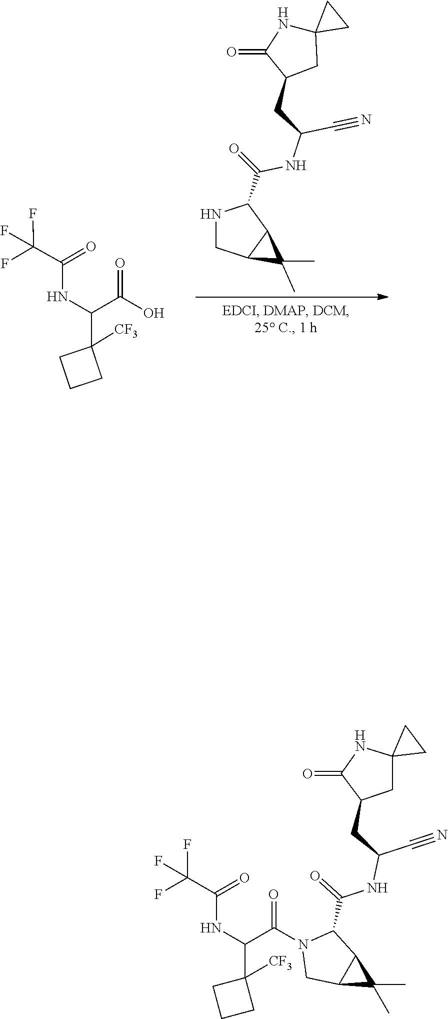 [SMILES: O=C(O)C(NC(=O)C(F)(F)F)C1(C(F)(F)F)CCC1 ; CC1(C)[C@@H]2[C@@H](C(=O)N[C@H](C#N)C[C@@H]3CC4(CC4)NC3=O)NC[C@@H]21 ; CC1(C)[C@@H]2[C@@H](C(=O)N[C@H](C#N)C[C@@H]3CC4(CC4)NC3=O)N(C(=O)C(NC(=O)C(F)(F)F)C3(C(F)(F)F)CCC3)C[C@@H]21]

To a solution of 2-[(2,2,2-trifluoroacetyl)amino]-2-[1-(trifluoromethyl)cyclobutyl]acetic acid (0.8 g, 2.73 mmol, 1 eq) and (1R,2S,5S)—N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (863.40 mg, 2.73 mmol, 1 eq) in DCM (15 mL) was added DMAP (666.77 mg, 5.46 mmol, 2 eq) and EDCI (1.05 g, 5.46 mmol, 2 eq). The mixture was stirred at 20° C. for 2 h. Upon completion, the reaction mixture was diluted with water (50 mL) and extracted with DCM (20 mL*3). The combined organic layers were dried over Na₂SO₄, filtered and concentrated under reduced pressure to give a residue. The residue was purified by prep-HPLC (column: Waters Xbridge Prep OBD C₁₋₈ 150*40 mm*10 um; mobile phase: [water(NH₄HCO₃)-ACN]; B %: 30%-60%, 8 min) to give (1R,2S,5S)—N-[(1S)-1-cyano-2-[(6R)-5-oxo-4-azaspiro[2.4]heptan-6-yl]ethyl]-6,6-dimethyl-3-[2-[(2,2,2-trifluoroacetyl)amino]-2-[1-(trifluoromethyl)cyclobutyl]acetyl]-3-azabicyclo[3.1.0]hexane-2-carboxamide (0.18 g, 293.36 umol, 10.75% yield, 96.408% purity) as a white solid. MS (ESI) m/z 592.2 [M+H]⁺

¹H NMR (400 MHz, DMSO-d₆) δ=10.12 (br d, J=6.6 Hz, 1H), 9.11 (br d, J=8.2 Hz, 1H), 7.77 (s, 1H), 5.02-4.90 (m, 1H), 4.86 (br d, J=6.2 Hz, 1H), 4.20 (s, 1H), 3.83 3.72 (m, 1H), 3.64 (br d, J=10.1 Hz, 1H), 2.78 (br d, J=8.9 Hz, 1H), 2.70-2.57 (m, 2H), 2.28-2.11 (m, 2H), 2.08-1.93 (m, 3H), 1.92-1.78 (m, 2H), 1.74-1.56 (m, 2H), 1.37 (br d, J=7.6 Hz, 1H), 1.04 (s, 3H), 0.96-0.82 (m, 3H), 0.79-0.69 (m, 1H), 0.65-0.49 (m, 3H).

Example ___: Synthesis of (1R,2S,5S)—N-[(1S)-1-cyano-2-[(6R)-5-oxo-4-azaspiro[2.4]heptan-6-yl]ethyl]-3-[(2S)-2-cyclopropyl-2-[(2,2,2-trifluoroacetyl)amino]acetyl]-6,6-dimethyl-3-azabicyclo[3.1.0]hexane-2-carboxamide (Compound 339)

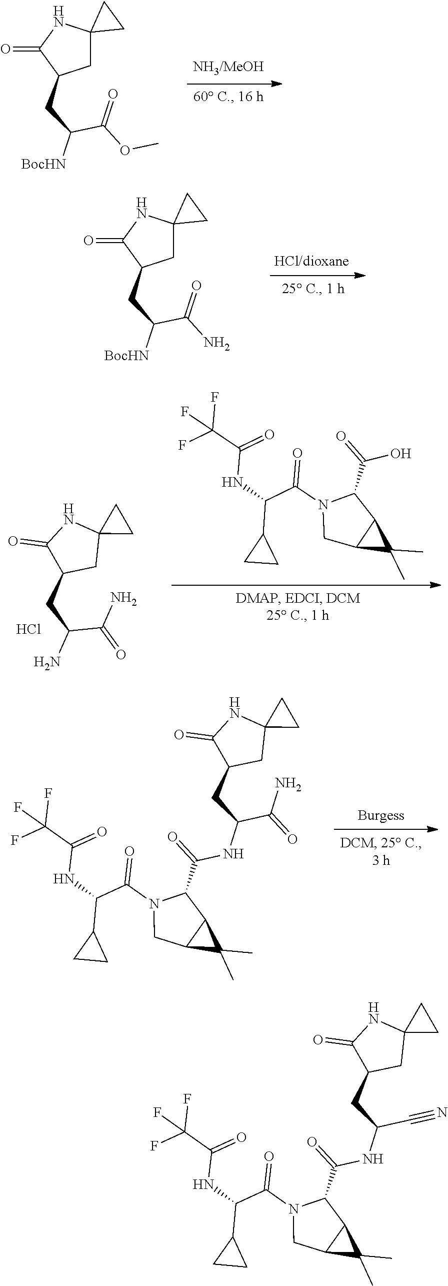

Step 1: tert-butyl N-[(1S)-2-amino-2-oxo-1-[[(6R)-5-oxo-4-azaspiro[2.4]heptan-6-yl]methyl]ethyl]carbamate

A solution of methyl (2S)-2-(tert-butoxycarbonylamino)-3-[(6R)-5-oxo-4-azaspiro[2.4]heptan-6-yl]propanoate (3 g, 9.60 mmol, 1 eq) in NH₃/MeOH (7 M, 34.29 mL, 24.99 eq) was stirred at 60° C. for 16 h. Upon completion, the reaction mixture was concentrated under reduced pressure to give the product tert-butyl N-[(1S)-2-amino-2-oxo-1-[[(6R)-5-oxo-4-azaspiro[2.4]heptan-6-yl]methyl]ethyl]carbamate (5.5 g, crude) as a yellow solid. MS (ESI) m/z 298.4 [M+H]⁺

Step 2: (2S)-2-amino-3-[(6R)-5-oxo-4-azaspiro[2.4]heptan-6-yl]propanamide

A solution of tert-butyl N-[(1S)-2-amino-2-oxo-1-[[(6R)-5-oxo-4-azaspiro[2.4]heptan-6-yl]methyl]ethyl]carbamate (500 mg, 1.68 mmol, 1 eq) in HCl/dioxane (4 M, 5 mL) was stirred at 25° C. for 1 h. Upon completion, the reaction mixture was concentrated under reduced pressure to give (2S)-2-amino-3-[(6R)-5-oxo-4-azaspiro[2.4]heptan-6-yl]propanamide (400 mg, crude, HCl salt) as a light yellow solid. MS (ESI) m/z 198.3 [M+H]⁺

Step 3: (1R,2S,5S)—N-[(1S)-2-amino-2-oxo-1-[[(6R)-5-oxo-4-azaspiro[2.4]heptan-6-yl]methyl]ethyl]-3-[(2S)-2-cyclopropyl-2-[(2,2,2-trifluoroacetyl)amino]acetyl]-6,6-dimethyl-3-azabicyclo[3.1.0]hexane-2-carboxamide

To a solution of (2S)-2-amino-3-[(6R)-5-oxo-4-azaspiro[2.4]heptan-6-yl]propanamide (370 mg, 1.58 mmol, 1 eq, HCl) in DCM (10 mL) was added (1R,2S,5S)-3-[(2S)-2-cyclopropyl-2-[(2,2,2-trifluoroacetyl)amino]acetyl]-6,6-dimethyl-3-azabicyclo[3.1.0]hexane-2-carboxylic acid (551.48 mg, 1.58 mmol, 1 eq), DMAP (580.28 mg, 4.75 mmol, 3 eq), and then was added EDCI (607.02 mg, 3.17 mmol, 2 eq). The resulting mixture was stirred at 25° C. for 1 h. Upon completion, the reaction mixture was diluted with H₂O (30 mL) and then was extracted with DCM (10 mL*3). The combined organic layers were was washed with HCl (1 M, 10 mL), then was adjusted pH-7 with sat.NaHCO₃(10 mL), dried over Na₂SO₄, filtered and concentrated in vacuum and was purified by column chromatography (SiO₂, Petroleum ether:Ethyl acetate=0:1 to Ethyl acetate:MeOH=10:1) to afford (1R,2S,5S)—N-[(1S)-2-amino-2-oxo-1-[[(6R)-5-oxo-4-azaspiro[2.4]heptan-6-yl]methyl]ethyl]-3-[(2S)-2-cyclopropyl-2-[(2,2,2-trifluoroacetyl)amino]acetyl]-6,6-dimethyl-3-azabicyclo[3.1.0]hexane-2-carboxamide (190 mg, 270.12 umol, 17.06% yield, 75% purity) as a yellow oil. MS (ESI) m/z 528.4 [M+H]⁺.

Step 4: (1R,2S,5S)—N-[(1S)-1-cyano-2-[(6R)-5-oxo-4-azaspiro[2.4]heptan-6-yl]ethyl]-3-[(2S)-2-cyclopropyl-2-[(2,2,2-trifluoroacetyl)amino]acetyl]-6,6-dimethyl-3-azabicyclo[3.1.0]hexane-2-carboxamide

To a solution of (1R,2S,5S)—N-[(1S)-2-amino-2-oxo-1-[[(6R)-5-oxo-4-azaspiro[2.4]heptan-6-yl]methyl]ethyl]-3-[(2S)-2-cyclopropyl-2-[(2,2,2-trifluoroacetyl)amino]acetyl]-6,6-dimethyl-3-azabicyclo[3.1.0]hexane-2-carboxamide (180 mg, 341.21 umol, 1 eq) in DCM (5 mL) was added burgess reagent (487.88 mg, 2.05 mmol, 6 eq), the mixture was stirred at 25° C. for 3 h. Upon completion, the reaction mixture was quenched by water (1 mL) and was dried by blowing N₂ and was purified by prep-HPLC (column: Waters Xbridge Prep OBD C18 150*40 mm*10 um; mobile phase: [water (NH₄HCO₃)-ACN]; B %: 30%-60%, 8 min) to obtained (1R,2S,5S)—N-[(1S)-1-cyano-2-[(6R)-5-oxo-4-azaspiro[2.4]heptan-6-yl]ethyl]-3-[(2S)-2-cyclopropyl-2-[(2,2,2-trifluoroacetyl)amino]acetyl]-6,6-dimethyl-3-azabicyclo[3.1.0]hexane-2-carboxamide (30 mg, 58.88 umol, 17.26% yield, 100% purity) as a white solid. MS (ESI) m/z 510.2 [M+H]⁺

¹H NMR (400 MHz, DMSO-d₆) δ=10.06-9.62 (m, 1H), 8.96 (d, J=8.4 Hz, 1H), 7.96-7.71 (m, 1H), 5.03-4.73 (m, 1H), 4.24-4.10 (m, 1H), 4.10-3.96 (m, 1H), 3.89 3.75 (m, 1H), 3.71-3.58 (m, 1H), 2.70-2.58 (m, 1H), 2.27-2.06 (m, 1H), 1.97 (d, J=9.0 Hz, 2H), 1.89-1.75 (m, 1H), 1.56 (dd, J=5.4, 7.4 Hz, 1H), 1.33-1.26 (m, 1H), 1.22-1.09 (m, 1H), 1.03 (s, 3H), 0.98-0.88 (m, 3H), 0.82-0.68 (m, 1H), 0.60-0.38 (m, 7H)

Example ___: Synthesis of (1R,2S,5S)—N-[(1S)-1-cyano-2-[(3R)-5,5-dimethyl-2-oxo-pyrrolidin-3-yl]ethyl]-3-[(2S)-2-cyclopropyl-2-[(2,2,2-trifluoroacetyl)amino]acetyl]-6,6-dimethyl-3-azabicyclo[3.1.0]hexane-2-carboxamide (Compound 338)

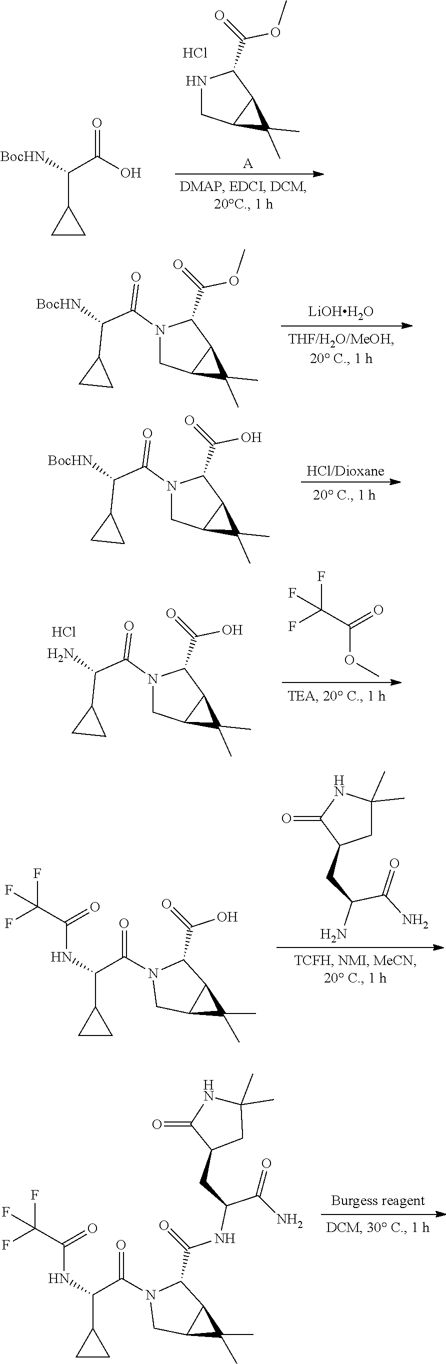

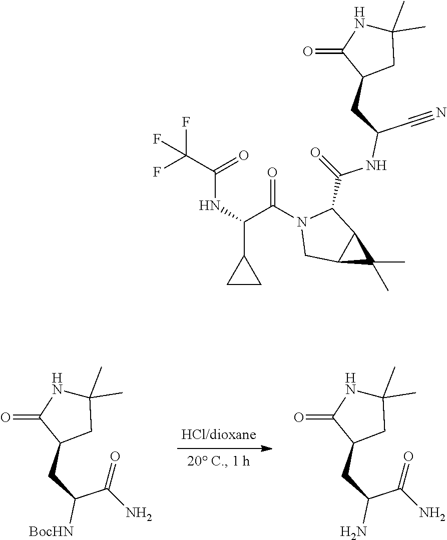 [SMILES: CC1(C)C[C@@H](C[C@@H](C#N)NC(=O)[C@@H]2[C@@H]3[C@H](CN2C(=O)[C@@H](NC(=O)C(F)(F)F)C2CC2)C3(C)C)C(=O)N1 ; CC1(C)C[C@@H](C[C@H](NC(=O)OC(C)(C)C)C(N)=O)C(=O)N1 ; CC1(C)C[C@@H](C[C@H](N)C(N)=O)C(=O)N1]

Step 1: methyl (2S)-2-amino-3-[(3R)-5,5-dimethyl-2-oxo pyrrolidin-3-yl]propanoate

The methyl (2S)-2-(tert-butoxycarbonylamino)-3-[(3R)-5,5-dimethyl-2-oxo-pyrrolidin-3-yl]propanoate (0.3 g, 954.27 umol, 1 eq) in HCl/dioxane (4 M, 3 mL, 12.58 eq) was stirred at 20° C. for 1 h. Upon completion, the reaction mixture was concentrated under reduced pressure to give the product methyl (2S)-2-amino-3-[(3R)-5,5-dimethyl-2-oxo-pyrrolidin-3-yl]propanoate (0.2 g, crude) as white solid. MS (ESI) m/z 200.1 [M+H]⁺

Step 2: methyl (1R,2S,5S)-3-[2-(tert-butoxycarbonylamino)-2-cyclopropyl-acetyl)-6,6-dimethyl-3-azabicyclo[3.1.0)hexane-2-carboxylate

To a solution of 2-(tert-butoxycarbonylamino)-2-cyclopropyl-acetic acid (2.5 g, 11.61 mmol, 1 eq), methyl (1R,2S,5S)-6,6-dimethyl-3-azabicyclo[3.1.0]hexane-2-carboxylate (2.39 g, 11.61 mmol, 1 eq, HCl) in DCM (20 mL) was added DMAP ((4.26 g, 34.84 mmol, 3 eq) and EDCI (4.45 g, 23.23 mmol, 2 eq). The mixture was stirred at 20° C. for 1 h. Upon completion, the reaction mixture was diluted with H₂O (80 mL) and then extracted with ethyl acetate (50 mL*3). The combined organic layers were dried over Na₂SO₄, filtered and concentrated under reduced pressure and was purified by prep-TLC (SiO₂, Petroleum ether:Ethyl acetate=2:1) to get product methyl (1R,2S,5S)-3-[2-(tert-butoxycarbonylamino)-2-cyclopropyl-acetyl]-6,6-dimethyl-3-azabicyclo[3.1.0]hexane-2-carboxylate (3.1 g, 8.29 mmol, 71.38% yield, 98% purity) as colorless oil. MS (ESI) m/z 367.4 [M+H]⁺

Step 3: (1R,2S,5S)-3-[2-(tert-butoxycarbonylamino)-2-cyclopropyl-acetyl)-6,6-dimethyl-3-azabicyclo[3.1.0]hexane-2-carboxylic acid

The methyl (1R,2S,5S)-3-[2-(tert-butoxycarbonylamino)-2-cyclopropyl-acetyl]-6,6-dimethyl-3-azabicyclo[3.1.0]hexane-2-carboxylate (3.1 g, 8.46 mmol, 1 eq) in THE (10 mL) and H₂O (10 mL) and MeOH (10 mL) was added LiOH.H₂O (1.06 g, 25.38 mmol, 3 eq). The mixture was stirred at 20° C. for 1 h. Upon completion, the mixture was adjusted pH-1 with HCl (aq. 1M) and then was extracted with EA (50 mL*3), then the organic phase was dried with Na₂SO₄, filtered and concentrated in vacuum to get crude product (1R,2S,5S)-3-[2-(tert-butoxycarbonylamino)-2-cyclopropyl-acetyl]-6,6-dimethyl-3-azabicyclo[3.1.0]hexane-2-carboxylic acid (2.8 g, crude) as yellow oil. MS (ESI) m/z 353.3 [M+H]⁺

Step 4: (1R,2S,5S)-3-[(2S)-2-amino-2-cyclopropyl-acetyl]-6,6-dimethyl-3-azabicyclo[3.1.0]hexane-2-carboxylic acid

The (1R,2S,5S)-3-[2-(tert-butoxycarbonylamino)-2-cyclopropyl-acetyl]-6,6-dimethyl-3-azabicyclo[3.1.0]hexane-2-carboxylic acid (2.8 g, 7.94 mmol, 1 eq) in HCl/dioxane (4 M, 35 mL, 17.62 eq) was stirred at 20° C. for 1 h. Upon completion, the reaction mixture was concentrated under reduced pressure to give (1R,2S,5S)-3-[(2S)-2-amino-2-cyclopropyl-acetyl]-6,6-dimethyl-3-azabicyclo[3.1.0]hexane-2-carboxylic acid (2 g, crude) as yellow oil. MS (ESI) m/z 253.3 [M+H]⁺

Step 5: (1R,2S,5S)-3-[(2S)-2-cyclopropyl-2-[(2,2,2-trifluoroacetyl)amino]acetyl]-6,6-dimethyl-3-azabicyclo[3.1.0]hexane-2-carboxylic acid

To a solution of (1R,2S,5S)-3-[(2S)-2-amino-2-cyclopropyl-acetyl]-6,6-dimethyl-3-azabicyclo[3.1.0]hexane-2-carboxylic acid (2 g, 7.93 mmol, 1 eq) in methyl 2,2,2-trifluoroacetate (30.45 g, 237.80 mmol, 23.98 mL, 30 eq) was added TEA (2.41 g, 23.78 mmol, 3.31 mL, 3 eq). The mixture was stirred at 20° C. for 1 h. Upon completion, the reaction mixture was concentrated under reduced pressure to give the crude product (1R,2S,5S)-3-[(2S)-2-cyclopropyl-2-[(2,2,2-trifluoroacetyl)amino]acetyl]-6,6-dimethyl-3-azabicyclo[3.1.0]hexane-2-carboxylic acid (2.8 g, crude) as yellow oil. MS (ESI) m/z 349.1 [M+H]⁺

Step 6: (1R,2S,5S)—N-[(1S)-2-amino-1-[[(3R)-5,5-dimethyl-2-oxo pyrrolidin-3-yl]methyl]-2-oxo-ethyl]-3-[(2S)-2-cyclopropyl-2-[(2,2,2-trifluoroacetyl)amino]acetyl]-6,6-dimethyl-3-azabicyclo[3.1.0]hexane-2-carboxamide

To a solution of (1R,2S,5S)-3-[(2S)-2-cyclopropyl-2-[(2,2,2-trifluoroacetyl)amino]acetyl]-6,6-dimethyl-3-azabicyclo[3.1.0]hexane-2-carboxylic acid (524.44 mg, 1.51 mmol, 1 eq) and (2S)-2-amino-3-[(3R)-5,5-dimethyl-2-oxo-pyrrolidin-3-yl]propanamide (0.3 g, 1.51 mmol, 1 eq) in ACN (10 mL) was added TCFH (844.91 mg, 3.01 mmol, 2 eq) and NMI (370.84 mg, 4.52 mmol, 360.04 uL, 3 eq). The mixture was stirred at 20° C. for 1 h. Upon completion, the reaction mixture was diluted with H₂O (60 mL) and then extracted with EA (40 mL*3). The combined organic layers were dried over Na₂SO₄, filtered and concentrated under reduced pressure and was purified by prep-MPLC (SiO₂, Petroleum ether:Ethyl acetate=0:1) to get product (1R,2S,5S)—N-[(1S)-2-amino-1-[[(3R)-5,5-dimethyl-2-oxo-pyrrolidin-3-yl]methyl]-2-oxo-ethyl]-3-[(2S)-2-cyclopropyl-2-[(2,2,2-trifluoroacetyl)amino]acetyl]-6,6-dimethyl-3-azabicyclo[3.1.0]hexane-2-carboxamide (0.3 g, 396.56 umol, 26.34% yield, 70% purity) as a white solid. MS (ESI) m/z 530.3 [M+H]⁺

Step 7: (1R,2S,5S)—N-[(1S)-1-cyano-2-[(3R)-5,5-dimethyl-2-oxo pyrrolidin-3-yl]ethyl]-3-[(2S)-2-cyclopropyl-2-[(2,2,2-trifluoroacetyl)amino]acetyl]-6,6-dimethyl-3-azabicyclo[3.1.0]hexane-2-carboxamide

To a solution of (1R,2S,5S)—N-[(1S)-2-amino-1-[[(3R)-5,5-dimethyl-2-oxo-pyrrolidin-3-yl]methyl]-2-oxo-ethyl]-3-[(2S)-2-cyclopropyl-2-[(2,2,2-trifluoroacetyl)amino]acetyl]-6,6-dimethyl-3-azabicyclo[3.1.0]hexane-2-carboxamide (0.3 g, 566.52 umol, 1 eq) in DCM (1 mL) was added burgess reagent (810.04 mg, 3.40 mmol, 6 eq).

The mixture was stirred at 30° C. for 1 h. Upon completion, the reaction mixture was quenched with water (1.5 mL) and blow-dried with N₂ and was purified by prep-HPLC (column: Waters Xbridge Prep OBD C18 150*44) mm*10 um; mobile phase: [water (NH₄HCO₃)-ACN]; B %: 30%-60%, 8 min) to get product (1R,2S,5S)—N-[(1S)-1-cyano-2-[(3R)-5,5-dimethyl-2-oxo-pyrrolidin-3-yl]ethyl]-3-[(2S)-2-cyclopropyl-2-[(2,2,2-trifluoroacetyl)amino]acetyl]-6,6-dimethyl-3-azabicyclo[3.1.0]hexane-2-carboxamide (0.1 g, 195.49 umol, 34.51% yield, 100% purity) as white solid. MS (ESI) m/z 512.2 [M+H]⁺

¹H NMR (400 MHz, DMSO-d₆) δ=9.89 (brd, J=5.6 Hz, 1H), 8.93 (d, J=8.1 Hz, 1H), 7.82 (s, 1H), 5.00-4.73 (m, 1H), 4.13 (s, 1H), 4.06-3.92 (m, 1H), 3.81 (dd, J=5.4, 10.3 Hz, 1H), 3.69-3.47 (m, 1H), 2.63-2.53 (m, 1H), 2.21-2.07 (m, 1H), 2.02 (dd, J=8.6, 12.2 Hz, 1H), 1.84-1.69 (m, 1H), 1.59-1.39 (m, 2H), 1.28 (d, J=7.6 Hz, 1H), 1.21-1.07 (m, 7H), 1.06-0.86 (m, 6H), 0.60-0.15 (m, 4H)

Example ___: Synthesis of (1R,2S,5S)—N-[(1S)-1-cyan-2-[(6R)-5-oxo-4-azaspiro[2.4]heptan-6-yl]ethyl]-3-[(2S)-3-(1-fluorocyclopropyl)-2-[(2,2,2-trifluoroacetyl)amino]propanoyl]-6,6-dimethyl-3-azabicyclo[3.1.0]hexane-2-carboxamide (Compound 337)

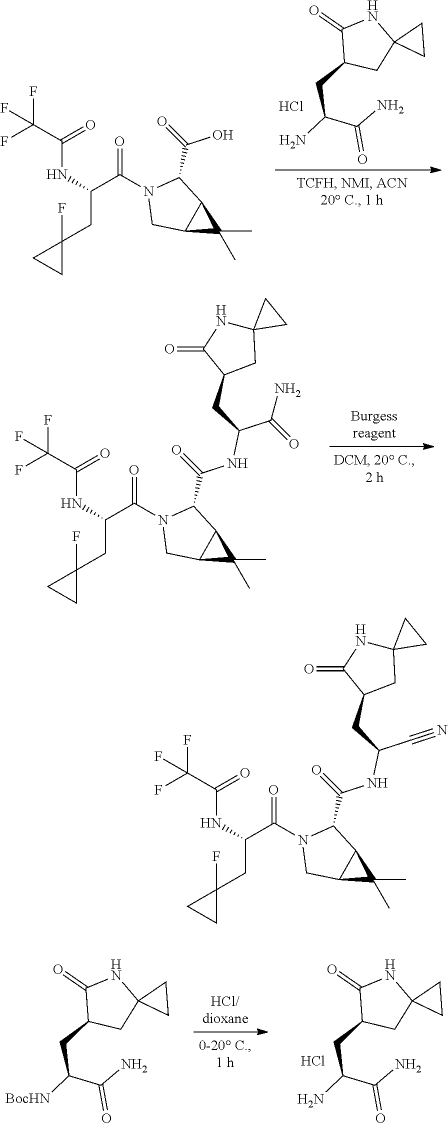

Step 1: (2S)-2-amino-3-[(6R)-5-oxo-4-azaspiro[2.4]heptan-6-yl]propanamide

A mixture of tert-butyl N-[(1S)-2-amino-2-oxo-1-[[(6R)-5-oxo-4-azaspiro[2.4]heptan-6-yl]methyl]ethyl]carbamate (550 mg, 1.85 mmol, 1 eq) was added HCl/dioxane (4 M, 8 mL, 17.30 eq) at 0° C., and the mixture was stirred at 20° C. for 1 h. The resulting mixture was concentrated in the vacuum to give (2S)-2-amino-3-[(6R)-5-oxo-4-azaspiro[2.4]heptan-6-yl]propanamide (470 mg, crude, HCl) as yellow solid. MS (ESI) m/z 198.1 [M+H]⁺.

Step 2: (1R,2S,5S)—N-[(1S)-2-amino-2-oxo-1-[[(6R)-5-oxo-4-azaspiro[2.4]heptan-6-yl]methyl]ethyl]-3-[(2S)-3-(1 fluorocyclopropyl)-2-[(2,2,2-trifluoroacetyl)amino]propanoyl]-6,6-dimethyl-3-azabicyclo[3.1.0]hexane-2-carboxamide

A solution of (1R,2S,5S)-3-[(2S)-3-(1-fluorocyclopropyl)-2-[(2,2,2-trifluoroacetyl)amino]propanoyl]-6,6-dimethyl-3-azabicyclo[3.1.0]hexane-2-carboxylic acid (700 mg, 1.66 mmol, 90% purity, 1 eq) in ACN (15 mL) was added with (2S)-2-amino-3-[(6R)-5-oxo-4-azaspiro[2.4]heptan-6-yl]propanamide (425.81 mg, 1.82 mmol, 1.1 eq, HCl), and then TCFH (929.53 mg, 3.31 mmol, 2 eq) and NMI (407.98 mg, 4.97 mmol, 396.10 uL, 3 eq) were added. The mixture was stirred at 20° C. for 1 h. Upon completion, the reaction was quenched by H₂O (30 mL), and was extracted with DCM (30 mL*3). The combined organic phase was washed with brine (50 mL), dried with anhydrous Na₂SO₄, filtered and concentrated in vacuum and purified by column chromatography (SiO₂, Petroleum ether:Ethyl acetate=1:0 to 3:7) to give (1R,2S,5S)—N-[(1S)-2-amino-2-oxo-1-[[(6R)-5-oxo-4-azaspiro[2.4]heptan-6-yl]methyl]ethyl]-3-[(2S)-3-(1-fluorocyclopropyl)-2-[(2,2,2-trifluoroacetyl)amino]propanoyl]-6,6-dimethyl-3-azabicyclo[3.1.0]hexane-2-carboxamide (400 mg, 714.86 umol, 43.16% yield) as yellow solid. MS (ESI) m/z 560.2 [M+H]⁺

Step 3: (1R,2S,5S)—N-[(1S)-1-cyano-2-[(6R)-5-oxo-4-azaspiro[2.4]heptan-6-yl]ethyl]-3-[(2S)-3-(1 fluorocyclopropyl)-2-[(2,2,2-trifluoroacetyl)amino]propanoyl]-6,6-dimethyl-3-azabicyclo[3.1.0]hexane-2-carboxamide

A solution of (1R,2S,5S)—N-[(1S)-2-amino-2-oxo-1-[[(6R)-5-oxo-4-azaspiro[2.4]heptan-6-yl]methyl]ethyl]-3-[(2S)-3-(1-fluorocyclopropyl)-2-[(2,2,2-trifluoroacetyl)amino]propanoyl]-6,6-dimethyl-3-azabicyclo[3.1.0]hexane-2-carboxamide (400 mg, 714.86 umol, 1 eq) in DCM (10 mL) was added burgess reagent (1.11 g, 4.65 mmol, 6.5 eq) and the mixture was stirred at 20° C. for 2 h. Upon completion, the mixture was quenched by water (1 mL) and was dried by blowing N₂ and purified by prep-HPLC (column: Phenomenex C18 80*40 mm*3 um; mobile phase: [water(NH₄HCO₃)-ACN]; B %: 25%-45%, 8 min) to give (1R,2S,5S)—N-[(1S)-1-cyano-2-[(6R)-5-oxo-4-azaspiro[2.4]heptan-6-yl]ethyl]-3-[(2S)-3-(1-fluorocyclopropyl)-2-[(2,2,2-trifluoroacetyl)amino]propanoyl]-6,6-dimethyl-3-azabicyclo[3.1.0]hexane-2-carboxamide (72.87 mg, 134.56 umol, 18.82% yield, 100% purity) as white solid. MS (ESI) m/z 542.2 [M+H]⁺

¹H NMR (400 MHz, DMSO-d₆) δ=9.95-9.51 (m, 1H), 8.93 (d, J=8.1 Hz, 1H), 7.78 (s, 1H), 5.00-4.60 (m, 2H), 4.14 (s, 1H), 3.89-3.85 (m, 1H), 3.70 (d, J=10.4 Hz, 1H), 2.65-2.50 (m, 1H), 2.36-2.24 (m, 1H), 2.22-2.01 (m, 2H), 1.97 (d, J=9.0 Hz, 2H), 1.91 1.78 (m, 1H), 1.65-1.53 (m, 1H), 1.33 (d, J=7.6 Hz, 1H), 1.06-1.01 (m, 3H), 0.98-0.90 (m, 2H), 0.88 (s, 3H), 0.79-0.70 (m, 2H), 0.63-0.49 (m, 4H)

Example ___: Synthesis of (1R,2S,5S)—N-[(1S)-1-cyano-2-[(6R)-5-oxo-4-azaspiro[2.4]heptan-6-yl]ethyl]-3-[(2S,3R)-3-(difluoromethoxy)-2-[(2,2,2-trifluoroacetyl)amino]butanoyl]-6,6-dimethyl-3-azabicyclo[3.1.0]hexane-2-carboxamide (Compound 321)

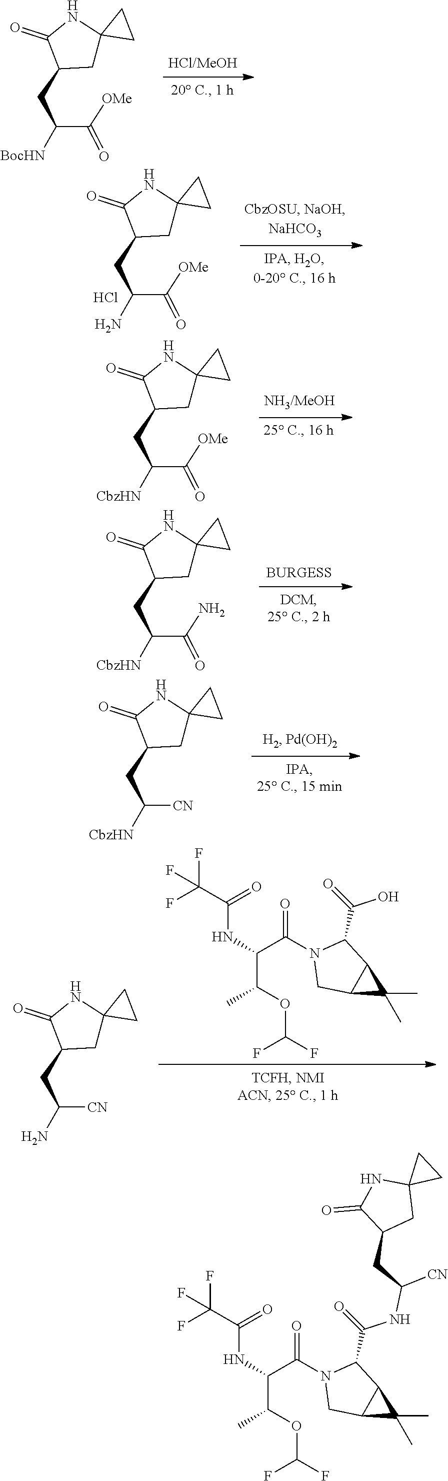

Step 1: methyl (2S)-2-amino-3-[(6R)-5-oxo-4-azaspiro[2.4]heptan-6-yl]propanoate

A solution of methyl (2S)-2-(tert-butoxycarbonylamino)-3-[(6R)-5-oxo-4-azaspiro[2.4]heptan-6-yl]propanoate (1 g, 3.20 mmol, 1 eq) in HCl/MeOH (20 mL) was stirred at 20° C. for 1 h. Upon completion, The reaction was concentrated under reduced pressure to give methyl (2S)-2-amino-3-[(6R)-5-oxo-4-azaspiro[2.4]heptan-6-yl]propanoate (700 mg, crude, HCl) as a white solid.

Step 2: methyl (2S)-2-(benzyloxycarbonylamino)-3-[(6R)-5-oxo-4-azaspiro[2.4]heptan-6-yl]propanoate

To a solution of methyl (2S)-2-amino-3-[(6R)-5-oxo-4-azaspiro[2.4]heptan-6-yl]propanoate (600 mg, 2.83 mmol, 1 eq) in IPA (10 mL), was added a solution of pH=11 of NaOH (113.08 mg, 2.83 mmol, 1 eq) and NaHCO₃(2.85 g, 33.92 mmol, 1.32 mL, 12 eq). Then, CbzOSu (774.97 mg, 3.11 mmol, 1.1 eq) was added at 0° C. The mixture was stirred at 20° C. for 16 h. Upon completion, the reaction mixture was diluted with H₂O (50 mL) and extracted with EA (20 mL*3). The combined organic layers were washed with brine (50 mL), dried over Na₂SO₄, filtered and concentrated under reduced pressure to give a residue. The residue was purified by column chromatography (SiO₂, Petroleum ether/Ethyl acetate=I/O to 1/1). Methyl (2S)-2-(benzyloxycarbonylamino)-3-[(6R)-5-oxo-4-azaspiro[2.4]heptan-6-yl]propanoate (830 mg, 2.16 mmol, 76.29% yield, 90% purity) was obtained as a white solid. MS (ESI) m/z 347.2 [M+H]⁺

Step 3: benzyl N-[(1S)-2-amino-2-oxo-1-[[(6R)-5-oxo-4-azaspiro[2.4]heptan-6-yl]methyl]ethyl]carbamate

A solution of methyl (2S)-2-(benzyloxycarbonylamino)-3-[(6R)-5-oxo-4-azaspiro[2.4]heptan-6-yl]propanoate (830 mg, 2.40 mmol, 1 eq) in NH₃/MeOH (20 mL, 7 M) and was stirred at 25° C. for 16 h. Upon completion, the mixture was concentrated under reduced pressure to afford benzyl N-[(1S)-2-amino-2-oxo-1-[[(6R)-5-oxo-4-azaspiro[2.4]heptan-6-yl]methyl]ethyl]carbamate (800 mg, crude) as a white solid. MS (ESI) m/z 332.2 [M+H]⁺

Step 4: benzyl N-[(1S)-1-cyano-2-[(6R)-5-oxo-4-azaspiro[2.4]heptan-6-yl]ethyl]carbamate

A solution of benzyl N-[(1S)-2-amino-2-oxo-1-[[(6R)-5-oxo-4-azaspiro[2.4]heptan-6-yl]methyl]ethyl]carbamate (300 mg, 905.34 umol, 1 eq) in DCM (5 mL) was added burgess reagent (1.29 g, 5.43 mmol, 6 eq) and then the resulting mixture was stirred at 25° C. for 2 h. Upon completion, the reaction mixture was concentrated under reduced pressure to give a residue. The residue was purified by prep-TLC (SiO₂, PE:EA=0:1) to afford benzyl N-[(1S)-1-cyano-2-[(6R)-5-oxo-4-azaspiro[2.4]heptan-6-yl]ethyl]carbamate (100 mg, 287.22 umol, 31.72% yield, 90% purity) as a white solid. MS (ESI) m/z 314.4 [M+H]⁺

Step 5: (2S)-2-amino-3-[(6R)-5-oxo-4-azaspiro[2.4]heptan-6-yl]propanenitrile

A solution of benzyl N-[(1S)-1-cyano-2-[(6R)-5-oxo-4-azaspiro[2.4]heptan-6-yl]ethyl]carbamate (100 mg, 319.13 umol, 1 eq) in IPA (5 mL) was added Pd(OH)₂/C (33.33 mg, 47.47 umol, 20% purity, 1.49e-1 eq). The resulting mixture was stirred under H₂ atmosphere at 25° C. for 15 min. Upon completion, the reaction mixture was filtered and concentrated under reduced pressure to give (2S)-2-amino-3-[(6R)-5-oxo-4-azaspiro[2.4]heptan-6-yl]propanenitrile (30 mg, crude) as a white solid.

Step 6: (1R,2S,5S)—N-[(1S)-1-cyano-2-[(6R)-5-oxo-4-azaspiro[2.4]heptan-6-yl]ethyl]-3-[(2S,3R)-3-(difluoromethoxy)-2-[(2,2,2-trifluoroacetyl)amino]butanoyl]-6,6-dimethyl-3-azabicyclo[3.1.0]hexane-2-carboxamide

A solution of (1R,2S,5S)-3-[(2S,3R)-3-(difluoromethoxy)-2-[(2,2,2-trifluoroacetyl)amino]butanoyl]-6,6-dimethyl-3-azabicyclo[3.1.0]hexane-2-carboxylic acid (33.67 mg, 83.70 umol, 1 eq) and (2S)-2-amino-3-[(6R)-5-oxo-4-azaspiro[2.4]heptan-6-yl]propanenitrile (15 mg, 83.70 umol, 1 eq) in ACN (2 mL) was added NMI (27.49 mg, 334.79 umol, 26.69 uL, 4 eq) then TCFH (28.18 mg, 100.44 umol, 1.2 eq) was added and the mixture was stirred at 25° C. for 1 h. Upon completion, the reaction mixture was diluted with H₂O (5 mL) and extracted with DCM (3 mL*3). The combined organic layers were washed with brine (5 mL), dried over Na₂SO₄, filtered and concentrated under reduced pressure to give a residue. The residue was purified by prep-HPLC (column: Waters Xbridge BEH C18 100*30 mm*10 um; mobile phase: [water(NH₄HCO₃)-ACN]; B %: 35%-65%, 8 min) to give(1R,2S,5S)—N-[(1S)-1-cyano-2-[(6R)-5-oxo-4-azaspiro[2.4]heptan-6-yl]ethyl]-3-[(2S,3R)-3-(difluoromethoxy)-2-[(2,2,2-trifluoroacetyl)amino]butanoyl]-6,6-dimethyl-3-azabicyclo[3.1.0]hexane-2-carboxamide (4.64 mg, 8.02 umol, 9.59% yield, 97.44% purity) as a white solid. MS (ESI) m/z 564.2 [M+H]⁺

¹H NMR (400 MHz, DMSO-d₆) δ=10.12 (br s, 1H), 9.05 (d, J=8.3 Hz, 1H), 8.13-7.70 (m, 1H), 6.67 (dd, J=72.9, 79.6 Hz, 1H), 5.02-4.85 (m, 1H), 4.58 (br d, J=9.2 Hz, 1H), 4.41 (br dd, J=6.4, 9.0 Hz, 1H), 4.21-4.07 (m, 1H), 3.97 (dd, J=5.4, 10.4 Hz, 1H), 3.79-3.67 (m, 1H), 2.69-2.58 (m, 1H), 2.22-2.11 (m, 1H), 2.06-1.70 (m, 3H), 1.58 (dd, J=5.5, 7.3 Hz, 1H), 1.38-1.33 (m, 1H), 1.32-1.26 (m, 3H), 1.03 (s, 3H), 0.85 (s, 3H), 0.84-0.61 (m, 2H), 0.60-0.50 (m, 2H)

Example ___: Synthesis of (1R,2S,5S)—N-[(1S)-1-cyano-2-[(3R)-5,5-dimethyl-2-oxo-pyrrolidin-3-yl]ethyl]-3-[(2S,3R)-3-(diiluoromethoxy)-2-[(2,2,2-trifluoroacetyl)amino]butanoyl]-6,6-dimethyl-3-azabicyclo[3.1.0]hexane-2-carboxamide (Compound 320)

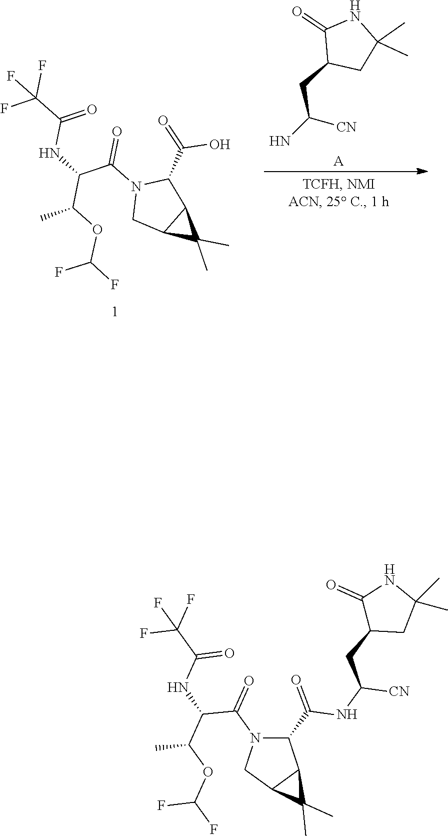

Step 1: (1R,2S,5S)—N-[(1S)-1-cyano-2-[(3R)-5,5-dimethyl-2-oxo pyrrolidin-3-yl]ethyl]-3-[(2S,3R)-3-(difluoromethoxy)-2-[(2,2,2-trifluoroacetyl)amino]butanoyl]-6,6-dimethyl-3-azabicyclo[3.1.0]hexane-2-carboxamide

A solution of (1R,2S,5S)-3-[(2S,3R)-3-(difluoromethoxy)-2-[(2,2,2-trifluoroacetyl)amino]butanoyl]-6,6-dimethyl-3-azabicyclo[3.1.0]hexane-2-carboxylic acid (66.60 mg, 165.53 umol, 1 eq) and (2S)-2-amino-3-[(3R)-5,5-dimethyl-2-oxo-pyrrolidin-3-yl]propanenitrile (30 mg, 165.53 umol, 1 eq) in ACN (2 mL) was added 1-methylimidazole (54.36 mg, 662.13 umol, 52.78 uL, 4 eq). Then, TCFH (55.73 mg, 198.64 umol, 1.2 eq) was added and the mixture was stirred at 25° C. for 1 h. Upon completion, the reaction mixture was diluted with H₂O (5 mL) and extracted with DCM (3 mL*3). The combined organic layers were washed with brine (5 mL), dried over Na₂SO₄, filtered and concentrated under reduced pressure to give a residue. The residue was purified by prep-HPLC (column: Waters Xbridge BEH C18 100*30 mm*10 um; mobile phase: [water (NH₄HCO₃)ACN]; B %: 35%-65%, 7 min) to afford (1R,2S,5S)—N-[(1S)-1-cyano-2-[(3R)-5,5-dimethyl-2-oxo-pyrrolidin-3-yl]ethyl]-3-[(2S,3R)-3-(difluoromethoxy)-2-[(2,2,2-trifluoroacetyl)amino]butanoyl]-6,6-dimethyl-3-azabicyclo[3.1.0]hexane-2-carboxamide (4.49 mg, 7.94 umol, 4.80% yield, 100% purity) as a white solid. MS (ESI) m/z 566.3 [M+H]⁺.

¹H NMR (400 MHz, DMSO-d₆) δ=10.01 (br d, J=3.4 Hz, 1H), 9.02 (d, J=8.4 Hz, 1H), 7.81 (s, 1H), 6.64 (dd, J=72.7, 79.7 Hz, 1H), 4.93 (ddd, J=5.6, 8.4, 10.1 Hz, 1H), 4.58 (d, J=9.3 Hz, 1H), 4.43-4.34 (m, 1H), 4.13 (s, 1H), 3.98 (dd, J=5.4, 10.4 Hz, 1H), 3.75 (d, J=10.5 Hz, 1H), 2.60-2.53 (m, 1H), 2.19-2.09 (m, 1H), 1.98 (dd, J=8.6, 12.3 Hz, 1H), 1.74 (ddd, J=5.6, 10.2, 13.5 Hz, 1H), 1.61-1.55 (m, 1H), 1.55-1.45 (m, 1H), 1.34 (d, J=7.6 Hz, 1H), 1.29 (d, J=6.1 Hz, 3H), 1.19 (s, 3H), 1.12 (s, 3H), 1.03 (s, 3H), 0.85 (s, 3H)

Example 70: Synthesis of (1R,2S,5S)—N-[(1S)-1-cyan-2-[(6R)-5-oxo-4-azaspiro[2.4]heptan-6-yl]ethyl]-3-[(2S)-3,3-dimethyl-2-(pyrimidin-2-ylamino)butanoyl]-6,6-dimethyl-3-azabicyclo[3.1.0]hexane-2-carboxamide (Compound 236)

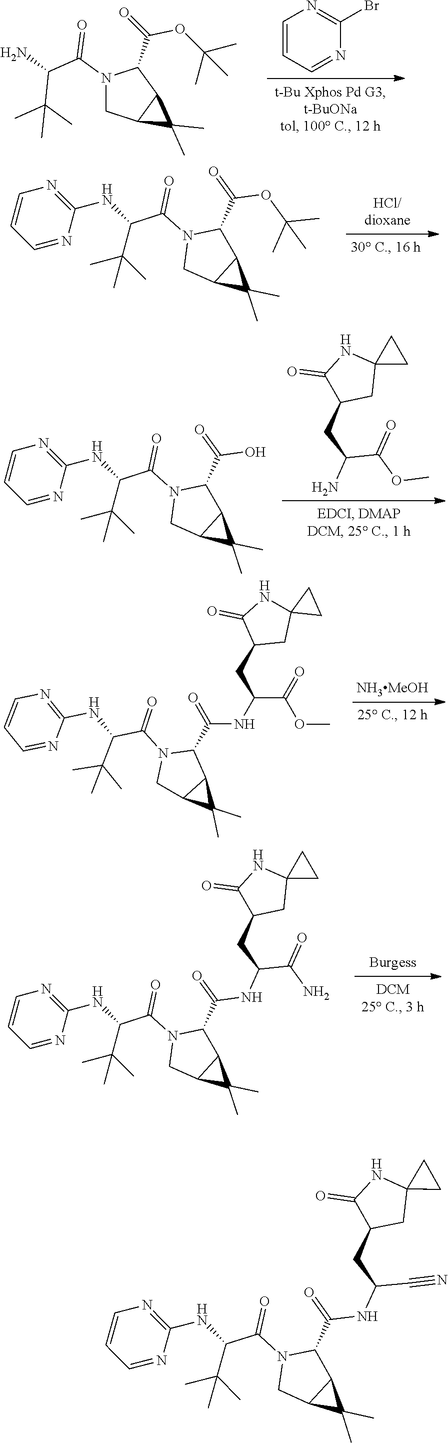

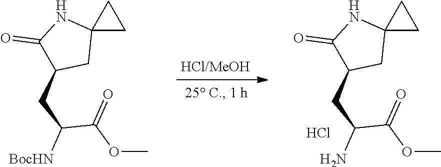 [SMILES: COC(=O)[C@H](C[C@@H]1CC2(CC2)NC1=O)NC(=O)OC(C)(C)C ; COC(=O)[C@@H](N)C[C@@H]1CC2(CC2)NC1=O ; Cl]

Step 1: methyl (2S)-2-amino-3-[(6R)-5-oxo-4-azaspiro[2.4]heptan-6-yl]propanoate

A mixture of methyl (2S)-2-(tert-butoxycarbonylamino)-3-[(6R)-5-oxo-4-azaspiro[2.4]heptan-6-yl]propanoate (300 mg, 960.43 umol, 1 eq) in HCl/MeOH (10 mL) (4 M) was stirred at 25° C. for 1 h. Upon completion, the reaction mixture was concentrated under reduced pressure to remove HCl/MeOH and added DCM (10 mL) (three times) was concentrated under reduced pressure to give methyl (2S)-2-amino-3-[(6R)-5-oxo-4-azaspiro[2.4]heptan-6-yl]propanoate (240 mg, crude, HCl) as a white oil. MS (ESI) m/z 213.2 [M+H]⁺.

Step 2: tert-butyl (1R,2S,5S)-3-[(2S)-3,3-dimethyl-2-(pyrimidin-2-ylamino)butanoyl]-6,6-dimethyl-3-azabicyclo[3.1.0]hexane-2-carboxylate

To a solution of tert-butyl (1R,2S,5S)-3-[(2S)-2-amino-3,3-dimethyl-butanoyl]-6,6-dimethyl-3-azabicyclo[3.1.0]hexane-2-carboxylate (250 mg, 770.52 umol, 1 eq), 2-bromopyrimidine (490.00 mg, 3.08 mmol, 4 eq) and t-BuONa (222.14 mg, 2.31 mmol, 3 eq) in toluene (3 mL) was added [2-(2-aminophenyl)phenyl]-methylsulfonyloxy-palladium; ditert-butyl-[2-(2,4,6-triisopropylphenyl)phenyl] phosphane (61.21 mg, 77.05 umol, 0.1 eq) under N₂. The mixture was stirred at 100° C. for 12 h under N₂. Upon completion, the reaction mixture was filtered and concentrated under reduced pressure to give a residue. The residue was purified by column chromatography (SiO₂, Petroleum ether:Ethyl acetate=1:0 to 3:1) to give tert-butyl (1R,2S,5S)-3-[(2S)-3,3-dimethyl-2-(pyrimidin-2-ylamino)butanoyl]-6,6-dimethyl-3-azabicyclo[3.1.0]hexane-2-carboxylate (300 mg, 603.68 umol, 39.17% yield, 81% purity) as a yellow oil. MS (ESI) m/z 403.3 [M+H]⁺.

Step 3: (1R,2S,5S)-3-[(2S)-3,3-dimethyl-2-(pyrimidin-2-ylamino)butanoyl]-6,6-dimethyl-3-azabicyclo[3.1.0]hexane-2-carboxylic acid

A mixture of tert-butyl (1R,2S,5S)-3-[(2S)-3,3-dimethyl-2-(pyrimidin-2-ylamino)butanoyl]-6,6-dimethyl-3-azabicyclo[3.1.0]hexane-2-carboxylate (455 mg, 1.13 mmol, 1 eq) in HCl/dioxane (4 M, 12.35 mL, 43.69 eq) was stirred at 30° C. for 16 h. Upon completion, the reaction mixture was concentrated under reduced pressure to remove HCl/dioxane and added DCM (15 mL) (three times) was concentrated under reduced pressure to give (1R,2S,5S)-3-[(2S)-3,3-dimethyl-2-(pyrimidin-2-ylamino)butanoyl]-6,6-dimethyl-3-azabicyclo[3.1.0]hexane-2-carboxylic acid (390 mg, crude) as a brown solid. MS (ESI) m/z 347.4 [M+H]⁺.

Step 4: methyl (2S)-2-[[(1R,2S,5S)-3-[(2S)-3,3-dimethyl-2-(pyrimidin-2-ylamino)butanoyl]-6,6-dimethyl-3-azabicyclo[3.1.0]hexane-2-carbonyl]amino]-3-[(6R)-5-oxo-4-azaspiro[2.4]heptan-6-yl]propanoate

To a solution of (1R,2S,5S)-3-[(2S)-3,3-dimethyl-2-(pyrimidin-2-ylamino)butanoyl]-6,6-dimethyl-3-azabicyclo[3.1.0]hexane-2-carboxylic acid (390 mg, 780.17 umol, 69.3% purity, 1 eq) and methyl (2S)-2-amino-3-[(6R)-5-oxo-4-azaspiro[2.4]heptan-6-yl]propanoate (194.03 mg, 780.17 umol, 1 eq, HCl) in DCM (5 mL) was added DMAP (190.62 mg, 1.56 mmol, 2 eq) and EDCI (224.34 mg, 1.17 mmol, 1.5 eq). The mixture was stirred at 25° C. for 1 h. Upon completion, the reaction was pour into H₂O (20 mL) and then extracted with DCM (15 mL*3). The combined organic phase was washed with brine (30 mL), dried over anhydrous Na₂SO₄, filtered and concentrated in vacuo. The residue was purified by column chromatography (SiO₂, Petroleum ether:Ethyl acetate=2:1 to Ethyl acetate:MeOH=4:1) to give methyl (2S)-2-[[(1R,2S,5S)-3-[(2S)-3,3-dimethyl-2-(pyrimidin-2-ylamino)butanoyl]-6,6-dimethyl-3-azabicyclo[3.1.0]hexane-2-carbonyl]amino]-3-[(6R)-5-oxo-4-azaspiro[2.4]heptan-6-yl]propanoate (350 mg, 563.21 umol, 72.19% yield, 87% purity) as a yellow solid. MS (ESI) m/z 541.5 [M+H]⁺.

Step 5: (1R,2S,5S)—N-[(1S)-2-amino-2-oxo-1-[[(6R)-5-oxo-4-azaspiro[2.4]heptan-6-yl]methyl]ethyl]-3-[(2S)-3,3-dimethyl-2-(pyrimidin-2-ylamino)butanoyl]-6,6-dimethyl-3-azabicyclo[3.1.0]hexane-2-carboxamide

A mixture of methyl (2S)-2-[[(1R,2S,5S)-3-[(2S)-3,3-dimethyl-2-(pyrimidin-2-ylamino)butanoyl]-6,6-dimethyl-3-azabicyclo[3.1.0]hexane-2-carbonyl]amino]-3-[(6R)-5-oxo-4-azaspiro[2.4]heptan-6-yl]propanoate (330 mg, 531.02 umol, 87% purity, 1 eq) in NH₃/MeOH (7 M, 15 mL, 197.73 eq) was stirred at 25° C. for 12 h. Upon completion, the reaction mixture was concentrated under reduced pressure to remove NH₃/MeOH and added DCM (15 mL) (three times) was concentrated under reduced pressure to give (1R,2S,5S)—N-[(1S)-2-amino-2-oxo-1-[[(6R)-5-oxo-4-azaspiro[2.4]heptan-6-yl]methyl]ethyl]-3-[(2S)-3,3-dimethyl-2-(pyrimidin-2-ylamino)butanoyl]-6,6-dimethyl-3-azabicyclo[3.1.0]hexane-2-carboxamide (300 mg, crude) as yellow solid. MS (ESI) m/z 526.6 [M+H]⁺.

Step 6: (1R,2S,5S)—N-[(1S)-1-cyano-2-[(6R)-5-oxo-4-azaspiro[2.4]heptan-6-yl]ethyl]-3-[(2S)-3,3-dimethyl-2-(pyrimidin-2-ylamino)butanoyl]-6,6-dimethyl-3-azabicyclo[3.1.0]hexane-2-carboxamide

To a solution of (1R,2S,5S)—N-[(1S)-2-amino-2-oxo-1-[[(6R)-5-oxo-4-azaspiro[2.4]heptan-6-yl]methyl]ethyl]-3-[(2S)-3,3-dimethyl-2-(pyrimidin-2-ylamino)butanoyl]-6,6-dimethyl-3-azabicyclo[3.1.0]hexane-2-carboxamide (300 mg, 570.73 umol, 1 eq) in DCM (5 mL) was added burgess reagent (408.03 mg, 1.71 mmol, 3 eq). The mixture was stirred at 25° C. for 3 h. Upon completion, the reaction mixture was quenched by addition H₂O 0.3 mL at 20° C., and then to remove DCM by N₂. The residue was purified by prep-HPLC (neutral condition; column:Waters Xbridge BEH C18 100*30 mm*10 um; mobile phase:[water (NH₄HCO₃)-ACN]; B %:25%-55%, 10 min) to afford (1R,2S,5S)—N-[(1S)-1-cyano-2-[(6R)-5-oxo-4-azaspiro[2.4]heptan-6-yl]ethyl]-3-[(2S)-3,3-dimethyl-2-(pyrimidin-2-ylamino)butanoyl]-6,6-dimethyl-3-azabicyclo[3.1.0]hexane-2-carboxamide (155 mg, 303.21 umol, 53.13% yield, 99.3% purity) as a white solid. MS (ESI) m/z 508.3 [M+H]⁺.

¹H NMR (400 MHz, DMSO-d₆) δ=9.21-8.85 (m, 1H), 8.36-8.18 (m, 2H), 7.98-7.64 (m, 1H), 6.74-6.57 (m, 2H), 5.03-4.75 (m, 1H), 4.61-4.35 (m, 1H), 4.27-3.97 (m, 2H), 3.95-3.54 (m, 1H), 2.69-2.61 (m, 1H), 2.24-2.14 (m, 1H), 2.01-1.89 (m, 2H), 1.84-1.74 (m, 1H), 1.60-1.55 (m, 1H), 1.26 (d, J=7.7 Hz, 1H), 1.11-0.94 (m, 12H), 0.94-0.81 (m, 3H), 0.77-0.70 (m, 1H), 0.65-0.47 (m, 3H)

¹H NMR (400 MHz, DMSO-d₆ (273+80K)) δ=8.81-8.66 (m, 1H), 8.34-8.21 (m, 2H), 7.49 (br s, 1H), 6.62 (q, J=4.8 Hz, 1H), 6.23-6.08 (m, 1H), 4.99-4.78 (m, 1H), 4.67-4.49 (m, 1H), 4.35-4.19 (m, 1H), 4.09-4.02 (m, 1H), 3.93 (td, J=4.8, 10.1 Hz, 1H), 2.67-2.65 (m, 1H), 2.28-2.18 (m, 1H), 2.10-2.02 (m, 1H), 2.01-1.92 (m, 1H), 1.90-1.81 (m, 1H), 1.59-1.54 (m, 1H), 1.29 (dd, J=4.3, 7.3 Hz, 1H), 1.15-0.95 (m, 12H), 0.93-0.73 (m, 4H), 0.68-0.52 (m, 3H).

Example 71: Synthesis of (1R,2S,5S)-3-((S)-2-amino-3,3-dimethylbutanoyl)-N—((S)-1-cyan-2-((R)-5-oxo-4-azaspiro[2.4]heptan-6-yl)ethyl)-6,6-dimethyl-3-azabicyclo[3.1.0]hexane-2-carboxamide (Compound 235)

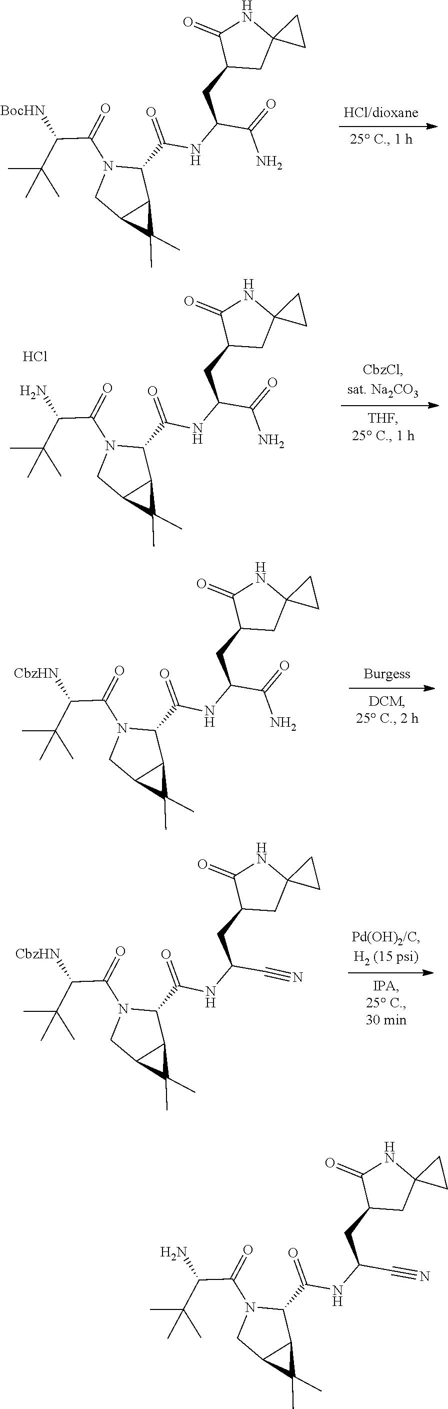

Step 1: (1R,2S,5S)-N—((S)-1-amino-1-oxo-3-((R)-5-oxo-4-azaspiro[2.4]heptan-6-yl)propan-2-yl)-3-((S)-2-amino-3,3-dimethylbutanoyl)-6,6-dimethyl-3-azabicyclo[3.1.0]hexane-2-carboxamide

A solution of tert-butyl N-[(1S)-1-[(1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl-propyl]carbamate (300 mg, 547.76 umol, 1 eq) in HCl/dioxane (4 M, 12 mL, 87.63 eq) was stirred at 25° C. for 1 h. The resulting mixture was concentrated under reduced pressure to remove solvent to give (1R,2S,5S)-3-[(2S)-2-amino-3,3-dimethyl-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide (265 mg, crude, HCl) as a yellow solid.

Step 2: benzyl ((S)-1-((1R,2S,5S)-2-(((S)-1-amino-1-oxo-3-((R)-5-oxo-4-azaspiro[2.4]heptan-6-yl)propan-2-yl)carbamoyl)-6,6-dimethyl-3-azabicyclo(3.1.0]hexan-3-yl)-3,3-dimethyl-1-oxobutan-2-yl)carbamate

To a solution of (1R,2S,5S)-3-[(2S)-2-amino-3,3-dimethyl-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide (265 mg, 547.49 umol, 1 eq, HCl) in THE (5 mL) was added sat. Na₂CO₃ (58.03 mg, 547.49 umol, 0.5 mL, 1 eq) (adjust pH-8), then CbzCl (186.79 mg, 1.09 mmol, 155.66 uL, 2 eq) was added. The mixture was stirred at 25° C. for 1 h. Upon complement, the reaction mixture was poured into H₂O 35 mL at 25° C., and then extracted with EtOAc (40 mL*3). The combined organic layers were washed with brine (30 mL*2), dried over Na₂SO₄, filtered and concentrated under reduced pressure to give a residue. The residue was purified by column chromatography (SiO₂, DCM:MeOH=100:0 to 95:5) to give benzyl N-[(1S)-1-[(1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl-propyl]carbamate (205 mg, 352.41 umol, 64.37% yield) as a white solid. MS (ESI) m/z 582.3 [M+H]⁺

Step 3: benzyl ((S)-1-((1R,2S,5S)-2-(((S)-1-cyano-2-((R)-5-oxo-4-azaspiro[2.4]heptan-6-yl)ethyl)carbamoyl)-6,6-dimethyl-3-azabicyclo(3.1.0]hexan-3-yl)-3,3-dimethyl-1-oxobutan-2-yl)carbamate

To a solution of benzyl N-[(1S)-1-[(1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl-propyl]carbamate (195 mg, 335.22 umol, 1 eq) in DCM (4 mL) was added burgess reagent (319.54 mg, 1.34 mmol, 4 eq). The mixture was stirred at 25° C. for 2 h. The resulting mixture was poured into H₂O 25 mL at 25° C., and then extracted with DCM (30 mL*3). The combined organic layers were washed with brine 30 mL, dried over Na₂SO₄, filtered and concentrated under reduced pressure to give a residue. The residue was purified by prep-HPLC (column: Waters Xbridge Prep OBD C18 150*40 mm*10 um; mobile phase: [water(NH₄HCO₃)-ACN]; B %: 35%-65%, 8 min) to give benzyl N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl-propyl]carbamate (40 mg, 70.96 umol, 21.17% yield) as a white solid. MS (ESI) m/z 564.3 [M+H]⁺

Step 4: (1R,2S,5S)-3-((S)-2-amino-3,3-dimethylbutanoyl)-N—((S)-1-cyano-2-((R)-5-oxo-4-azaspiro[2.4]heptan-6-yl)ethyl)-6,6-dimethyl-3-azabicyclo[3.1.0]hexane-2-carboxamide

To a solution of benzyl N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl-propyl]carbamate (35 mg, 62.09 umol, 1 eq) in IPA (2 mL) was added Pd(OH)₂ (35 mg, 49.85 umol, 20% purity, 8.03e-1 eq) under N₂. The suspension was degassed under vacuum and purged with H₂ several times. The mixture was stirred under H₂ (15 psi) at 25° C. for 30 min. The resulting reaction mixture was filtered and concentrated under reduced pressure to give a residue. The residue was purified by prep-HPLC (column: Phenomenex Luna C18 75*30 mm*3 um; mobile phase: [water(FA)-ACN]; B %: 1%-40%, 8 min) to afford (1R,2S,5S)-3-[(2S)-2-amino-3,3-dimethyl-butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (17 mg, 34.49 umol, 55.55% yield, 96.5% purity, FA) as a white solid. MS (ESI) m/z 430.3 [M+H]⁺.

¹H NMR (400 MHz, MeOD-d₄) δ=8.49 (s, 1H), 5.02 (d, J=5.6 Hz, 1H), 4.33 (s, 1H), 4.03-3.93 (m, 1H), 3.80-3.67 (m, 2H), 2.93-2.81 (m, 1H), 2.42-2.32 (m, 1H), 2.16 (d, J=8.9 Hz, 1H), 2.09 (d, J=9.0 Hz, 1H), 2.01-1.91 (m, 1H), 1.65 (s, 1H), 1.43 (d, J=7.6 Hz, 1H), 1.13-1.00 (m, 15H), 0.89-0.83 (m, 1H), 0.77-0.60 (m, 3H).

Example 72: Synthesis of (1R,2S,5S)-3-((S)-2-amino-3,3-dimethylbutanoyl)-N—((S)-1-cyan-2-((S)-2-oxopyrrolidin-3-yl)ethyl)-6,6-dimethyl-3-azabicyclo[3.1.0]hexane-2-carboxamide (Compound 234)

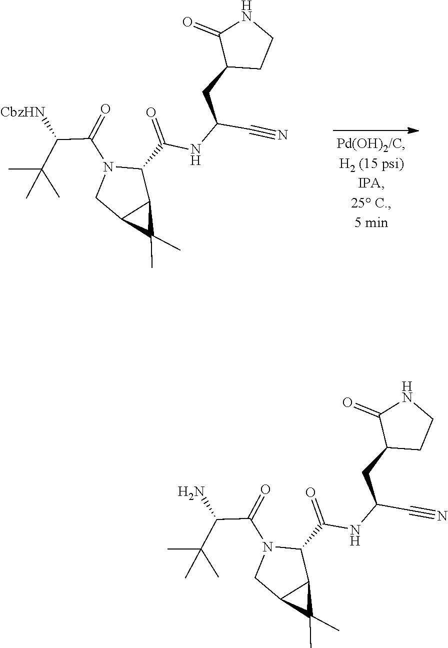 [SMILES: CC(C)(C)[C@H](NC(=O)OCc1ccccc1)C(=O)N1C[C@H]2[C@@H]([C@H]1C(=O)N[C@H](C#N)C[C@@H]1CCNC1=O)C2(C)C ; CC(C)(C)[C@H](N)C(=O)N1C[C@H]2[C@@H]([C@H]1C(=O)N[C@H](C#N)C[C@@H]1CCNC1=O)C2(C)C]

Step 1: (1R,2S,5S)-3-((S)-2-amino-3,3-dimethylbutanoyl)-N—((S)-1-cyano-2-((S)-2-oxopyrrolidin-3-yl)ethyl)-6,6-dimethyl-3-azabicyclo[3.1.0]hexane-2-carboxamide

To a solution of benzyl N-[(1S)-1-[(1R,2S,5S)-2-[[(1S)-1-cyano-2-[(3S)-2-oxopyrrolidin-3-yl]ethyl]carbamoyl]-6,6-dimethyl-3-azabicyclo[3.1.0]hexane-3-carbonyl]-2,2-dimethyl-propyl]carbamate (40 mg, 74.40 umol, 1 eq) in IPA (3 mL) was added Pd(OH)₂ (40.00 mg, 56.97 umol, 20% purity, 7.66e-1 eq) under N₂. The suspension was degassed under vacuum and purged with H₂ several times. The mixture was stirred under H₂ (15 psi) at 25° C. for 5 min. Upon completion, the reaction mixture was filtered and filtrate concentrated under reduced pressure to give a residue. The residue was purified by prep-HPLC (column: Phenomenex Luna C18 75*30 mm*3 um; mobile phase: [water(FA)-ACN]; B %: 1%-40%, 8 min) to give (1R,2S,5S)-3-[(2S)-2-amino-3,3-dimethyl-butanoyl]-N-[(1S)-1-cyano-2-[(3S)-2-oxopyrrolidin-3-yl]ethyl]-6,6-dimethyl-3-azabicyclo[3.1.0]hexane-2-carboxamide (10 mg, 24.53 umol, 32.98% yield, 99% purity) as a white solid. MS (ESI) m/z 404.1 [M+H]⁺.

¹H NMR (400 MHz, MeOD-d₄) δ=8.48 (s, 1H), 5.05 (dd, J=4.9, 10.9 Hz, 1H), 4.33 (s, 1H), 3.99 (dd, J=5.7, 10.4 Hz, 1H), 3.82 (s, 1H), 3.73 (d, J=10.5 Hz, 1H), 3.31 3.18 (m, 2H), 2.69-2.56 (m, 1H), 2.38-2.24 (m, 2H), 1.94-1.77 (m, 2H), 1.67 (dd, J=5.6, 7.5 Hz, 1H), 1.44 (d, J=7.6 Hz, 1H), 1.10 (s, 12H), 1.05-1.02 (m, 3H).

Example 73: Synthesis of (1R,2S,5S)—N-[(1S)-1-cyan-2-[(6R)-5-oxo-4-azaspiro[2.4]heptan-6-yl]ethyl]-3-[3,3-dimethyl-2-[(2,2,2-trifluoro-1-methyl-ethyl)amino]butanoyl]-6,6-dimethyl-3-azabicyclo[3.1.0]hexane-2-carboxamide (Compound 230)

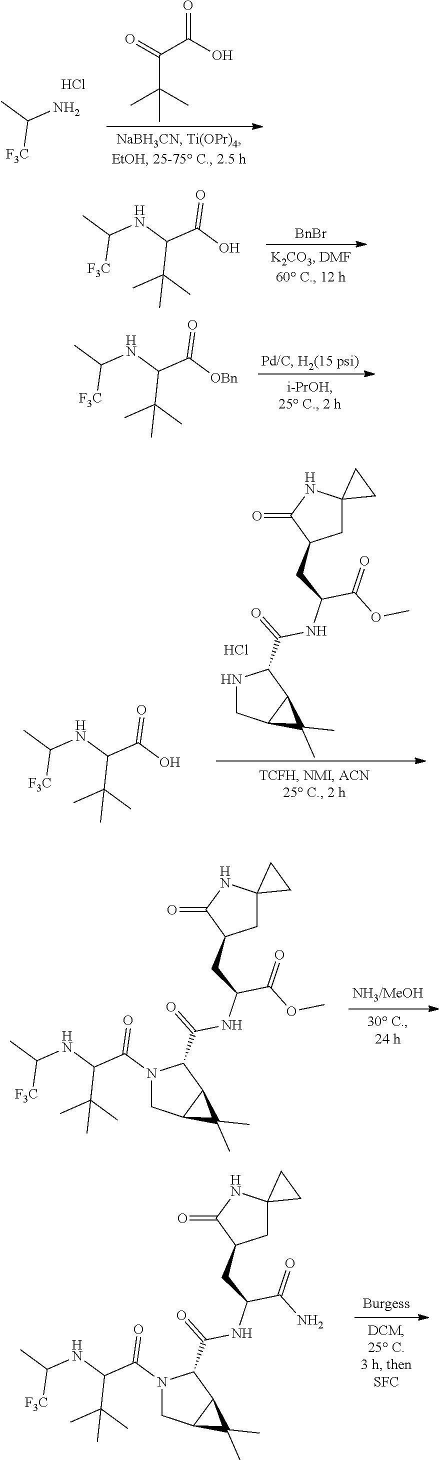

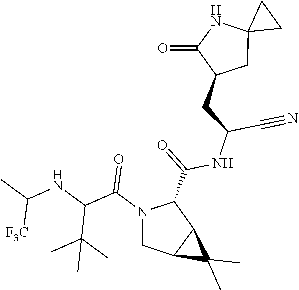 [SMILES: CC(NC(C(=O)N1C[C@H]2[C@@H]([C@H]1C(=O)N[C@H](C#N)C[C@@H]1CC3(CC3)NC1=O)C2(C)C)C(C)(C)C)C(F)(F)F]

Step 1: 3,3-dimethyl-2-[(2,2,2-trifluoro-1-methyl-ethyl)amino]butanoic acid

To a mixture of 1,1,1-trifluoropropan-2-amine (1 g, 6.69 mmol, 1 eq, HCl) and 3,3-dimethyl-2-oxo-butanoic acid (870.26 mg, 6.69 mmol, 1 eq) at 25° C. was added Ti(i-PrO)₄ (8.46 g, 29.76 mmol, 8.78 mL, 4.45 eq). The solution was warmed to 75° C. for about min. The solution was diluted with absolute EtOH (10 mL) at 25° C., followed by the addition of NaBH₃CN (1.08 g, 17.12 mmol, 2.56 eq), and the reaction was stirred at 25° C. for 2 h, Upon completion, the reaction was quenched with water(15 mL) white solid formed, and EtOH(20 mL) was added, filtered, and washed with MeOH(10 mL*4), concentrated in vacuum to give 3,3-dimethyl-2-[(2,2,2-trifluoro-1-methyl-ethyl)amino]butanoic acid (4 g, crude) was obtained as a white solid.(Batches two)

Step 2: benzyl 3,3-dimethyl-2-[(2,2,2-trifluoro-1-methyl-ethyl)amino]butanoate

To a solution of 3,3-dimethyl-2-[(2,2,2-trifluoro-1-methyl-ethyl)amino]butanoic acid (4 g, 17.60 mmol, 1 eq) in DMF (40 mL) was added K₂CO₃ (7.30 g, 52.81 mmol, 3 eq) and bromomethylbenzene (3.46 g, 20.21 mmol, 2.40 mL, 1.15 eq). The resulting mixture was heated in an oil bath at 60° C. and was stirred for 12 h. Upon completion, saturated aqueous NH₄Cl (80 mL) was added to stop the reaction, then was extracted twice with EA (30 mL*2). The combined organic phase was washed with brine(30 mL*4), dried over Na₂SO₄ and concentrated in vacuum. The combined crude product was purified by silica gel chromatography (SiO₂, PE:EA=99:1), then prep-TLC(PE:EA=10:1) to give benzyl 3,3-dimethyl-2-[(2,2,2-trifluoro-1-methyl-ethyl)amino]butanoate (400 mg, 1.26 mmol, 7.16% yield) as a colorless liquid.

Step 3: 3,3-dimethyl-2-[(2,2,2-trifluoro-1-methyl-ethyl)amino]butanoic acid

To a solution of benzyl 3,3-dimethyl-2-[(2,2,2-trifluoro-1-methyl-ethyl)amino]butanoate (400 mg, 1.26 mmol, 1 eq) in i-PrOH (5 mL) was added wet. Pd/C (0.1 g, 10% purity) under N₂ atmosphere. The suspension was degassed and purged with H₂ for 3 times. The mixture was stirred under H₂ (190.48 mg, 94.30 mmol, 74.81 eq) (15 Psi) at 25° C. for 2 h. Upon completion, the reaction mixture was filtered to get the filtrate and combined and concentrated to give 3,3-dimethyl-2-[(2,2,2-trifluoro-1-methyl-ethyl)amino]butanoic acid (300 mg, crude) as a white solid.

Step 4: methyl (2S)-2-[[(1R,2S,5S)-3-[3,3-dimethyl-2-[(2,2,2-trifluoro-1-methyl-ethyl)amino]butanoyl]-6,6-dimethyl-3-azabicyclo[3.1.0]hexane-2-carbonyl]amino]-3-[(6R)-5-oxo-4-azaspiro[2.4]heptan-6-yl]propanoate

To a solution of 3,3-dimethyl-2-[(2,2,2-trifluoro-1-methyl-ethyl)amino]butanoic acid (150 mg, 660.14 umol, 1 eq) and methyl (2S)-2-[[(1R,2S,5S)-6,6-dimethyl-3-azabicyclo[3.1.0]hexane-2-carbonyl]amino]-3-[(6R)-5-oxo-4-azaspiro[2.4]heptan-6-yl]propanoate (254.74 mg, 660.14 umol, 1 eq, HCl) in DMF (5 mL) was added 1-methylimidazole (189.69 mg, 2.31 mmol, 184.17 uL, 3.5 eq) and [chloro(dimethylamino)methylene]-dimethyl-ammonium; hexafluorophosphate (203.74 mg, 726.16 umol, 1.1 eq), and then the mixture was stirred at 25° C. for 2 h. Upon completion, the residue was poured into water (20 mL). The aqueous phase was extracted with ethyl acetate (20 mL*2). The combined organic phase was washed with brine (10 mL*2), dried with anhydrous Na₂SO₄, filtered and concentrated in vacuum. The residue was purified by silica gel chromatography (SiO₂, Petroleum ether:Ethyl acetate=1:1 to EA) to give methyl (2S)-2-[[(1R,2S,5S)-3-[3,3-dimethyl-2-[(2,2,2-trifluoro-1-methyl-ethyl)amino]butanoyl]-6,6-dimethyl-3-azabicyclo[3.1.0]hexane-2-carbonyl]amino]-3-[(6R)-5-oxo-4-azaspiro[2.4]heptan-6-yl]propanoate (170 mg, 304.31 umol, 46.10% yield) as a light yellow solid.

Step 5: (1R,2S,5S)—N-[(1S)-2-amino-2-oxo-1-[[(6R)-5-oxo-4-azaspiro[2.4]heptan-6-yl]methyl]ethyl]-3-[3,3-dimethyl-2-[(2,2,2-trifluoro-1-methyl-ethyl)amino]butanoyl]-6,6-dimethyl-3-azabicyclo[3.1.0]hexane-2-carboxamide

A solution of methyl (2S)-2-[[(1R,2S,5S)-3-[3,3-dimethyl-2-[(2,2,2-trifluoro-1-methyl-ethyl)amino]butanoyl]-6,6-dimethyl-3-azabicyclo[3.1.0]hexane-2-carbonyl]amino]-3-[(6R)-5-oxo-4-azaspiro[2.4]heptan-6-yl]propanoate (510 mg, 912.94 umol, 1 eq) in NH₃.MeOH (7 M, 10 mL, 76.67 eq) was stirred at 30° C. fo 24 h. Upon completion, the reaction mixture was concentrated under reduced pressure to give (1R,2S,5S)—N-[(1S)-2-amino-2-oxo-1-[[(6R)-5-oxo-4-azaspiro[2.4]heptan-6-yl]methyl]ethyl]-3-[3,3-dimethyl-2-[(2,2,2-trifluoro-1-methyl-ethyl)amino]butanoyl]-6,6-dimethyl-3-azabicyclo[3.1.0]hexane-2-carboxamide (500 mg, crude) as a light yellow solid.

Step 6: (1R,2S,5S)—N-[(1S)-1-cyano-2-[(6R)-5-oxo-4-azaspiro[2.4]heptan-6-yl]ethyl]-3-[3,3-dimethyl-2-[(2,2,2-trifluoro-1-methyl-ethyl)amino]butanoyl]-6,6-dimethyl-3-azabicyclo[3.1.0]hexane-2-carboxamide

To a solution of (1R,2S,5S)—N-[(1S)-2-amino-2-oxo-1-[[(6R)-5-oxo-4-azaspiro[2.4]heptan-6-yl]methyl]ethyl]-3-[3,3-dimethyl-2-[(2,2,2-trifluoro-1-methyl-ethyl)amino]butanoyl]-6,6-dimethyl-3-azabicyclo[3.1.0]hexane-2-carboxamide (540 mg, 993.34 umol, 1 eq) in DCM (10 mL) was added burgess reagent (710.17 mg, 2.98 mmol, 3 eq) and then the mixture was stirred at 25° C. for 3 h. Upon completion, the residue was quenched with water (0.5 mL) and was stirred for 10 min, then was concentrated bellow 30° C. The residue was purified by prep-HPLC column: Waters Xbridge Prep OBD C18 150*40 mm*10 um; mobile phase: [water(NH₄HCO₃)-ACN]; B %: 45%-75%, 8 min to give the pure product 100 mg, then was purified by SFC column: DAICEL CHIRALPAK AD(250 mm*30 mm, 10 um); mobile phase: [0.1% NH₃H₂O IPA]; B %: 10%-30%, 10 min to give P1 Rt=1.925 min (1R,2S,5S)—N-[(1S)-1-cyano-2-[(6R)-5-oxo-4-azaspiro[2.4]heptan-6-yl]ethyl]-3-[3,3-dimethyl-2-[(2,2,2-trifluoro-1-methyl-ethyl)amino]butanoyl]-6,6-dimethyl-3-azabicyclo[3.1.0]hexane-2-carboxamide Isomer 1 (35 mg, 65.39 umol, 6.58% yield, 98.2% purity) as a white solid. MS (ESI) m/z 526.4 [M+H]⁺.

¹H NMR (400 MHz, DMSO-d₆) δ=9.15-8.92 (m, 1H), 7.76 (s, 1H), 4.90 (ddd, J=6.1, 8.2, 10.3 Hz, 1H), 4.19-4.12 (m, 1H), 3.82 (dd, J=5.4, 10.3 Hz, 1H), 3.67-3.60 (m, 1H), 3.29-3.23 (m, 1H), 3.13 (d, J=11.3 Hz, 1H), 2.71-2.60 (m, 1H), 2.18 (ddd, J=4.9, 10.4, 13.6 Hz, 1H), 2.06 (br d, J=11.3 Hz, 1H), 1.93 (dd, J=2.3, 8.9 Hz, 2H), 1.83 (ddd, J=5.8, 10.6, 13.5 Hz, 1H), 1.59 (dd, J=5.5, 7.4 Hz, 1H), 1.27 (d, J=7.6 Hz, 1H), 1.16 (d, J=6.4 Hz, 3H), 1.03 (s, 3H), 0.95 (s, 3H), 0.89 (s, 9H), 0.77-0.69 (m, 1H), 0.61 0.53 (m, 2H), 0.52-0.45 (m, 1H)

(1R,2S,5S)—N-[(1S)-1-cyano-2-[(6R)-5-oxo-4-azaspiro[2.4]heptan-6-yl]ethyl]-3-[3,3-dimethyl-2-[(2,2,2-trifluoro-1-methyl-ethyl)amino]butanoyl]-6,6-dimethyl-3-azabicyclo[3.1.0]hexane-2-carboxamide Isomer 2 was obtained (38 mg, 72.30 umol, 7.28% yield, 100% purity) as a white solid. MS (ESI) m/z 526.4 [M+H]⁺.

¹H NMR (400 MHz, DMSO-d₆) δ=9.06 (d, J=8.6 Hz, 1H), 7.74 (s, 1H), 5.01 4.83 (m, 1H), 4.20 (s, 1H), 3.85 (dd, J=5.5, 10.5 Hz, 1H), 3.60 (d, J=10.6 Hz, 1H), 3.25 3.06 (m, 2H), 2.73-2.61 (m, 1H), 2.26-2.11 (m, 2H), 2.05-1.87 (m, 2H), 1.80 (ddd, J=5.1, 10.9, 13.4 Hz, 1H), 1.63-1.48 (m, 1H), 1.30 (d, J=7.6 Hz, 1H), 1.13 (d, J=6.7 Hz, 3H), 1.04 (s, 3H), 0.94-0.87 (m, 12H), 0.76-0.69 (m, 1H), 0.60-0.49 (m, 3H)

Example 74: Synthesis of (1R,2S,5S)—N-[(1S)-1-cyan-2-[(6R)-5-oxo-4-azaspiro[2.4]heptan-6-yl]ethyl]-3-[(2S)-2-(2,4-difluoroanilino)-3,3-dimethyl-butanoyl]-6,6-dimethyl-3-azabicyclo[3.1.0]hexane-2-carboxamide (Compound 228)

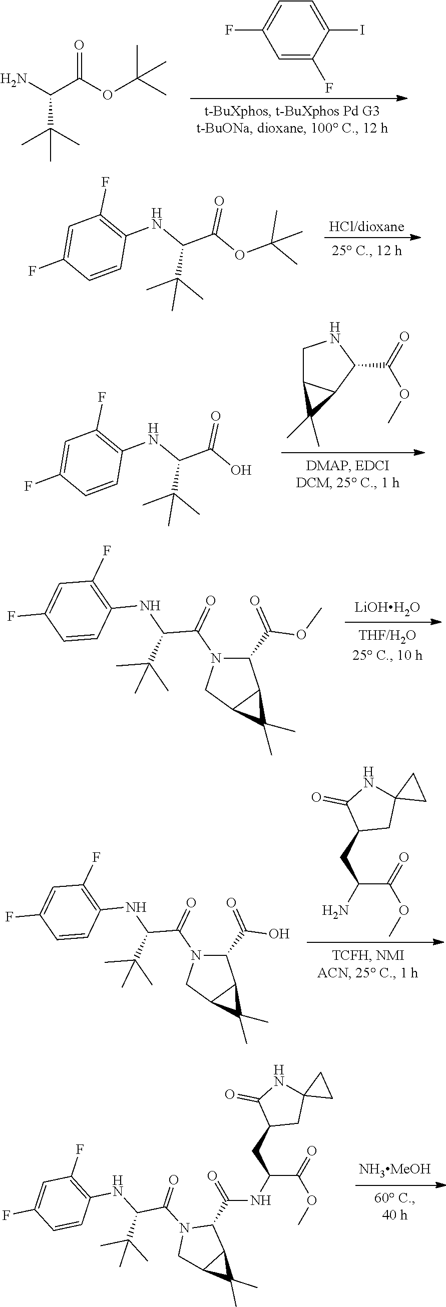

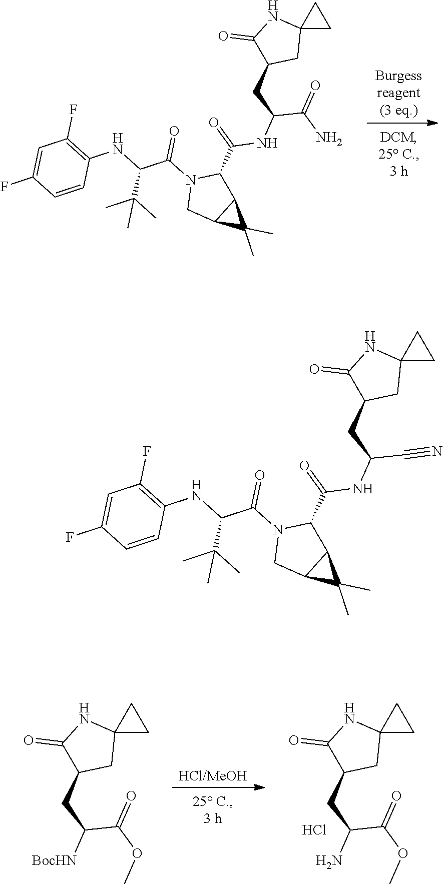

Step 1: methyl (2S)-2-amino-3-[(6R)-5-oxo-4-azaspiro[2.4]heptan-6-yl]propanoate

A solution of methyl (2S)-2-(tert-butoxycarbonylamino)-3-[(6R)-5-oxo-4-azaspiro[2.4]heptan-6-yl]propanoate (300 mg, 960.43 umol, 1 eq) in HCl/MeOH (4 M, 6.00 mL, 24.99 eq) was stirred at 25° C. for 1 h. Upon completion, the mixture was concentrated in the vacuum to give methyl (2S)-2-amino-3-[(6R)-5-oxo-4-azaspiro[2.4]heptan-6-yl]propanoate (300 mg, crude, HCl) was yellow solid. MS (ESI) m/z 213.1 [M+H]⁺.

Step 2: tert-butyl (2S)-2-(2,4-difluoroanilino)-3,3-dimethyl-butanoate

To a mixture of tert-butyl (2S)-2-amino-3,3-dimethyl-butanoate (1.06 g, 5.68 mmol, 1 eq) and 2,4-difluoro-1-iodo-benzene (1.5 g, 6.25 mmol, 1.1 eq) was added dioxane (30 mL) under glove box, then was added t-BuONa (1.09 g, 11.36 mmol, 2 eq), then t-Bu Xphos (241.28 mg, 568.21 umol, 0.1 eq), followed by [2-(2-aminophenyl)phenyl]-methylsulfonyloxy-palladium; ditert-butyl-[2-(2,4,6-triisopropylphenyl)phenyl]phosphene (451.37 mg, 568.21 umol, 0.1 eq) and the mixture was stirred at 100° C. for 12 h. Upon completion, the two batch reaction mixture were filtered through diatomite, and added H₂O (100 mL). The mixture was extracted with ethyl acetate (100 mL*3). The organic phase was washed with brine (300 mL), dried over anhydrous Na₂SO₄, concentrated in vacuum to give a residue. The residue was purified by column chromatography (SiO₂, Petroleum ether:Ethyl acetate=96:4 to 94:6) to give tert-butyl (2S)-2-(2,4-difluoroanilino)-3,3-dimethyl-butanoate (400 mg, 935.34 umol, 8.23% yield, 70% purity) as black brown oil. MS (ESI) m/z 300.2 [M+H]⁺.

Step 3: (2S)-2-(2,4-difluoroanilino)-3,3-dimethyl-butanoic acid

A solution of tert-butyl (2S)-2-(2,4-difluoroanilino)-3,3-dimethyl-butanoate (400 mg, 1.34 mmol, 1 eq) in HCl/dioxane (4 M, 8.00 mL, 23.95 eq) was stirred at 25° C. for 12 h. Upon completion, the reaction was concentrated in the vacuum and was purified by prep-HPLC (column: Phenomenex Luna 80*30 mm*3 um; mobile phase: [water(HCl)-ACN]; B %: 40%-60%, 8 min) to give (2S)-2-(2,4-difluoroanilino)-3,3-dimethyl-butanoic acid (180 mg, 739.98 umol, 55.38% yield) as yellow oil. MS (ESI) m/z 244.1 [M+H]⁺.

Step 4: methyl (1R,2S,5S)-3-[(2S)-2-(2,4-difluoroanilino)-3,3-dimethyl-butanoyl]-6,6-dimethyl-3-azabicyclo[3.1.0]hexane-2-carboxylate

To a solution of (2S)-2-(2,4-difluoroanilino)-3,3-dimethyl-butanoic acid (180 mg, 739.98 umol, 1 eq) in DCM (4 mL) was added methyl (1R,2S,5S)-6,6-dimethyl-3-azabicyclo[3.1.0]hexane-2-carboxylate (137.74 mg, 813.98 umol, 1.1 eq), DMAP (271.21 mg, 2.22 mmol, 3 eq), followed by EDCI (283.71 mg, 1.48 mmol, 2 eq). The resulting mixture was stirred at 25° C. for 1 h. Upon completion, the reaction was quenched by H₂O (10 mL), and was extracted with DCM (10 mL*3). The combined organic phase was washed with brine (30 mL), dried with anhydrous Na₂SO₄, filtered, concentrated in vacuum and purified by prep-TLC (SiO₂, PE:EA=3:1) to give methyl (1R,2S,5S)-3-[(2S)-2-(2,4-difluoroanilino)-3,3-dimethyl-butanoyl]-6,6-dimethyl-3-azabicyclo[3.1.0]hexane-2-carboxylate (100 mg, 253.51 umol, 34.26% yield) as yellow solid. MS (ESI) m/z 395.2 [M+H]⁺.

Step 5: (1R,2S,5S)-3-[(2S)-2-(2,4-difluoroanilino)-3,3-dimethyl-butanoyl]-6,6-dimethyl-3-azabicyclo[3.1.0]hexane-2-carboxylic acid

A solution of methyl (1R,2S,5S)-3-[(2S)-2-(2,4-difluoroanilino)-3,3-dimethyl-butanoyl]-6,6-dimethyl-3-azabicyclo[3.1.0]hexane-2-carboxylate (160 mg, 405.62 umol, 1 eq) in THE (4 mL) and H₂O (2 mL) was added LiOH.H₂O (51.06 mg, 1.22 mmol, 3 eq) and the mixture was stirred at 25° C. for 10 h. Upon completion, the reaction was concentrated in the vacuum to give (1R,2S,5S)-3-[(2S)-2-(2,4-difluoroanilino)-3,3-dimethyl-butanoyl]-6,6-dimethyl-3-azabicyclo[3.1.0]hexane-2-carboxylic acid (400 mg, crude, Li) as yellow solid. MS (ESI) m/z 381.2 [M+H]⁺.

Step 6: methyl (2S)-2-[[(1R,2S,5S)-3-[(2S)-2-(2,4-difluoroanilino)-3,3-dimethyl-butanoyl]-6,6-dimethyl-3-azabicyclo[3.1.0]hexane-2-carbonyl]amino]-3-[(6R)-5-oxo-4-azaspiro[2.4]heptan-6-yl]propanoate

To a solution of (1R,2S,5S)-3-[(2S)-2-(2,4-difluoroanilino)-3,3-dimethyl-butanoyl]-6,6-dimethyl-3-azabicyclo[3.1.0]hexane-2-carboxylic acid (400 mg, 1.03 mmol, 1 eq, Li) in ACN (8 mL) was added methyl (2S)-2-amino-3-[(6R)-5-oxo-4-azaspiro[2.4]heptan-6-yl]propanoate (282.50 mg, 1.14 mmol, 1.1 eq, HCl), then [chloro(dimethylamino)methylene]-dimethyl-ammonium; hexafluorophosphate (289.73 mg, 1.03 mmol, 1 eq), followed by 1-methylimidazole (254.34 mg, 3.10 mmol, 246.93 uL, 3 eq) and the mixture was stirred at 25° C. for 1 h. The reaction was quenched by H₂O (20 mL), and extracted with DCM (10 mL*3). The combined organic phase was washed with brine (30 mL), dried with anhydrous Na₂SO₄, filtered and concentrated in vacuum. The residue was purified by column chromatography (SiO₂, Petroleum ether:Ethyl acetate=1:4 to 1:10) to give methyl (2S)-2-[[(1R,2S,5S)-3-[(2S)-2-(2,4-difluoroanilino)-3,3-dimethyl-butanoyl]-6,6-dimethyl-3-azabicyclo[3.1.0]hexane-2-carbonyl]amino]-3-[(6R)-5-oxo-4-azaspiro[2.4]heptan-6-yl]propanoate (200 mg, 348.03 umol, 33.70% yield) as yellow solid. MS (ESI) m/z 575.3 [M+H]⁺.

Step 7: (1R,2S,5S)—N-[(1S)-2-amino-2-oxo-1-[[(6R)-5-oxo-4-azaspiro[2.4]heptan-6-yl]methyl]ethyl]-3-[(2S)-2-(2,4-dif luoroanilino)-3,3-dimethyl-butanoyl]-6,6-dimethyl-3-azabicyclo[3.1.0]hexane-2-carboxamide

A solution of methyl (2S)-2-[[(1R,2S,5S)-3-[(2S)-2-(2,4-difluoroanilino)-3,3-dimethyl-butanoyl]-6,6-dimethyl-3-azabicyclo[3.1.0]hexane-2-carbonyl]amino]-3-[(6R)-5-oxo-4-azaspiro[2.4]heptan-6-yl]propanoate (200 mg, 348.03 umol, 1 eq) in NH₃/MeOH (7 M, 10 mL, 201.13 eq) was stirred at 60° C. for 40 h. Upon completion, the reaction was concentrated in the vacuum to give (1R,2S,5S)—N-[(1S)-2-amino-2-oxo-1-[[(6R)-5-oxo-4-azaspiro[2.4]heptan-6-yl]methyl]ethyl]-3-[(2S)-2-(2,4-difluoroanilino)-3,3-dimethyl-butanoyl]-6,6-dimethyl-3-azabicyclo[3.1.0]hexane-2-carboxamide (200 mg, crude) as yellow solid. MS (ESI) m/z 560.3 [M+H]⁺.

Step 8: (1R,2S,5S)—N-[(1S)-1-cyano-2-[(6R)-5-oxo-4-azaspiro[2.4]heptan-6-yl]ethyl]-3-[(2S)-2-(2,4-difluoroanilino)-3,3-dimethyl-butanoyl]-6,6-dimethyl-3-azabicyclo[3.1.0]hexane-2-carboxamide

A solution of (1R,2S,5S)—N-[(1S)-2-amino-2-oxo-1-[[(6R)-5-oxo-4-azaspiro[2.4]heptan-6-yl]methyl]ethyl]-3-[(2S)-2-(2,4-difluoroanilino)-3,3-dimethyl-butanoyl]-6,6-dimethyl-3-azabicyclo[3.1.0]hexane-2-carboxamide (200 mg, 357.37 umol, 1 eq) in DCM (4 mL) was added burgess reagent (170.33 mg, 714.74 umol, 2.0 eq) and the mixture was stirred at 25° C. for 3 h. Upon completion, the mixture was quenched by H₂O (0.5 mL), concentrated in the vacuum, purified by prep-HPLC (column: Phenomenex C18 80*40 mm*3 um; mobile phase: [water(NH₄HCO₃)-ACN]; B %: 37%-57%, 8 min) and separated by SFC (column: DAICEL CHIRALCEL OD(250 mm*30 mm, 10 um); mobile phase: [0.1% NH₃H₂O MEOH]; B %: 14%-14%, 6 min) to give (1R,2S,5S)—N-[(1S)-1-cyano-2-[(6R)-5-oxo-4-azaspiro[2.4]heptan-6-yl]ethyl]-3-[(2S)-2-(2,4-difluoroanilino)-3,3-dimethyl-butanoyl]-6,6-dimethyl-3-azabicyclo[3.1.0]hexane-2-carboxamide Isomer 1 (17 mg, 29.87 umol, 8.36% yield, 95.18% purity).

¹H NMR (400 MHz, DMSO-d₆) δ=9.32-8.81 (m, 1H), 7.96-7.67 (m, 1H), 7.14-7.04 (m, 1H), 7.03-6.96 (m, 1H), 6.92-6.83 (m, 1H), 4.96-4.77 (m, 1H), 4.35-4.19 (m, 1H), 4.08 (s, 1H), 4.00 (br d, J=9.8 Hz, 1H), 3.76-3.67 (m, 2H), 2.72-2.67 (m, 1H), 2.19-2.07 (m, 1H), 1.96-1.85 (m, 2H), 1.83-1.75 (m, 1H), 1.59-1.48 (m, 1H), 1.24-1.21 (m, 1H), 1.03-0.89 (m, 15H), 0.79-0.71 (m, 1H), 0.64-0.48 (m, 3H).

¹H NMR (400 MHz, DMSO+D20-d6) δ=7.10-6.93 (m, 2H), 6.92-6.80 (m, 1H), 4.82-4.79 (m, 1H), 4.06 (s, 1H), 3.97 (s, 1H), 3.71 (br s, 2H), 2.65-2.68 (m, 1H), 2.19-2.00 (m, 1H), 1.95-1.71 (m, 3H), 1.56-1.47 (m, 1H), 1.22-1.17 (m, 1H), 1.03-0.88 (m, 15H), 0.79-0.71 (m, 1H), 0.65-0.44 (m, 3H)

(1R,2S,5S)—N-[(1S)-1-cyano-2-[(6R)-5-oxo-4-azaspiro[2.4]heptan-6-yl]ethyl]-3-[(2S)-2-(2,4-difluoroanilino)-3,3-dimethyl-butanoyl]-6,6-dimethyl-3-azabicyclo[3.1.0]hexane-2-carboxamide Isomer 2 was obtained as (41 mg, 75.70 umol, 21.18% yield, 100% purity) as white solid MS (ESI) m/z 542.2 [M+H]⁺.

¹H NMR (400 MHz, DMSO-d₆) δ=9.00 (d, J=8.5 Hz, 1H), 7.75 (s, 1H), 7.13-6.96 (m, 2H), 6.90-6.81 (m, 1H), 4.94-4.88 (m, 1H), 4.39 (br dd, J=2.9, 11.4 Hz, 1H), 4.04 (s, 1H), 3.99 (br d, J=11.4 Hz, 1H), 3.84 (dd, J=5.4, 10.6 Hz, 1H), 3.64 (br d, J=10.8 Hz, 1H), 2.70-2.61 (m, 1H), 2.24-2.12 (m, 1H), 2.03-1.88 (m, 2H), 1.84-1.75 (m, 1H), 1.52 (dd, J=5.5, 7.4 Hz, 1H), 1.23 (d, J=7.5 Hz, 1H), 1.09-0.88 (m, 12H), 0.77-0.71 (m, 1H), 0.64-0.52 (m, 3H), 0.47 (s, 3H)

¹H NMR (400 MHz, DMSO+D2O-d₆) δ=7.13-6.95 (m, 2H), 6.86-6.81 (m, 1H), 4.90 (dd, J=5.1, 10.6 Hz, 1H), 4.03 (s, 1H), 3.99-3.95 (m, 1H), 3.84 (br dd, J=5.3, 10.6 Hz, 1H), 3.62 (br d, J=10.8 Hz, 1H), 2.69-2.59 (m, 1H), 2.24-2.11 (m, 1H), 2.02 1.88 (m, 2H), 1.84-1.73 (m, 1H), 1.54-1.49 (m, 1H), 1.21 (d, J=7.7 Hz, 1H), 1.03-0.93 (m, 12H), 0.80-0.70 (m, 1H), 0.61-0.50 (m, 3H), 0.45 (s, 3H)

Example 75: Synthesis of (1R,2S,5S)—N-[(1S)-1-cyan-2-[(6R)-5-oxo-4-azaspiro[2.4]heptan-6-yl]ethyl]-3-[(2S)-3,3-dimethyl-2-(thiazol-2-ylamino)butanoyl]-6,6-dimethyl-3-azabicyclo[3.1.0]hexane-2-carboxamide (Compound 227)

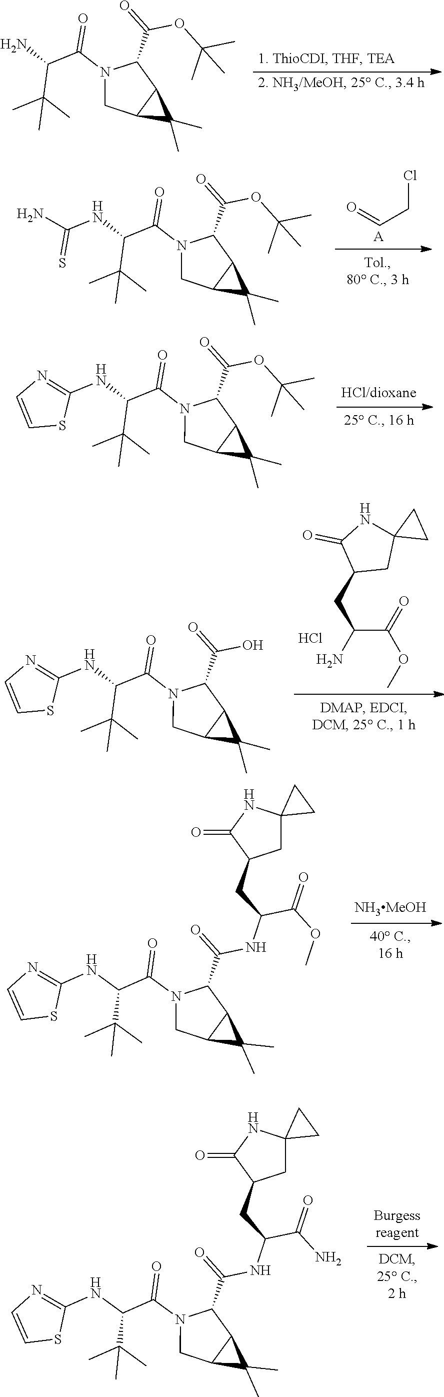

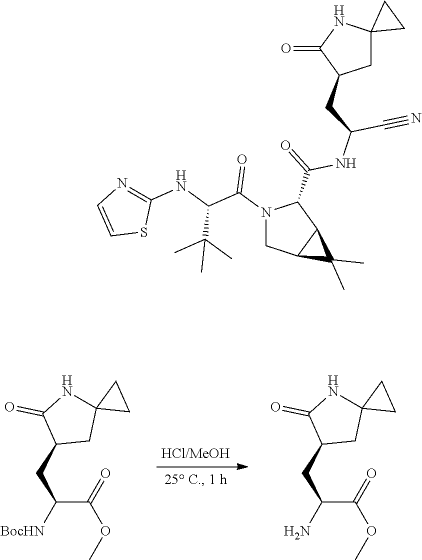 [SMILES: CC(C)(C)[C@H](Nc1nccs1)C(=O)N1C[C@H]2[C@@H]([C@H]1C(=O)N[C@H](C#N)C[C@@H]1CC3(CC3)NC1=O)C2(C)C ; COC(=O)[C@H](C[C@@H]1CC2(CC2)NC1=O)NC(=O)OC(C)(C)C ; COC(=O)[C@@H](N)C[C@@H]1CC2(CC2)NC1=O]

Step 1: methyl (2S)-2-amino-3-[(6R)-5-oxo-4-azaspiro[2.4]heptan-6-yl]propanoate

A solution of methyl (2S)-2-(tert-butoxycarbonylamino)-3-[(6R)-5-oxo-4-azaspiro[2.4]heptan-6-yl]propanoate (650 mg, 2.08 mmol, 1 eq) in HCl/MeOH (4 M, 10 mL, 19.22 eq) was stirred at 25° C. for 1 h. Upon completion, the mixture was concentrated in the vacuum to give methyl (2S)-2-amino-3-[(6R)-5-oxo-4-azaspiro[2.4]heptan-6-yl]propanoate (600 mg, crude, HCl) as yellow solid. MS (ESI) m/z 213.1 [M+H]⁺

Step 2: tert-butyl (1R,2S,5S)-3-[(2S)-2-(carbamothioylamino)-3,3-dimethyl-butanoyl]-6,6-dimethyl-3-azabicyclo[3.1.0]hexane-2-carboxylate

To a solution of tert-butyl (1R,2S,5S)-3-[(2S)-2-amino-3,3-dimethyl-butanoyl]-6,6-dimethyl-3-azabicyclo[3.1.0]hexane-2-carboxylate (600 mg, 1.85 mmol, 1 eq) in THE (10 mL) was added TEA (467.81 mg, 4.62 mmol, 643.48 uL, 2.5 eq) and the mixture was stirred for 0.4 h, then was added di(imidazol-1-yl)methanethione (494.34 mg, 2.77 mmol, 1.5 eq) and stirred for 1 h, at last the reaction was added NH₃/MeOH (7 M, 1.06 mL, 4 eq) and stirred at 25° C. for 2 h. Upon completion, the mixture was concentrated in the vacuum, and quenched by H₂O (5 mL), and was extracted with DCM (5 mL*3). The combined organic phase was washed with brine (20 mL), dried with anhydrous Na₂SO₄, filtered and concentrated in vacuum to give tert-butyl (1R,2S,5S)-3-[(2S)-2-(carbamothioylamino)-3,3-dimethyl-butanoyl]-6,6-dimethyl-3-azabicyclo[3.1.0]hexane-2-carboxylate (680 mg, crude) as yellow oil. MS (ESI) m/z 384.2 [M+H]⁺

Step 3: tert-butyl (1R,2S,5S)-3-[(2S)-3,3-dimethyl-2-(thiazol-2-ylamino)butanoyl]-6,6-dimethyl-3-azabicyclo[3.1.0]hexane-2-carboxylate

A solution of tert-butyl (1R,2S,5S)-3-[(2S)-2-(carbamothioylamino)-3,3-dimethyl-butanoyl]-6,6-dimethyl-3-azabicyclo[3.1.0]hexane-2-carboxylate (680 mg, 1.77 mmol, 1 eq) in toluene (20 mL) was added 2-chloroacetaldehyde (347.93 mg, 1.77 mmol, 285.18 uL, 40% purity, 1 eq) and the mixture was stirred at 80° C. for 3 h. Upon completion, the reaction was added NaOH (20%, aq.) to adjust pH=7 and was extracted with DCM (20 mL*3). The combined organic phase was washed with brine (20 mL), dried with anhydrous Na₂SO₄, filtered, concentrated in vacuum and purified by column chromatography (SiO₂, Petroleum ether:Ethyl acetate=7:3 to 2:1) to give tert-butyl (1R,2S,5S)-3-[(2S)-3,3-dimethyl-2-(thiazol-2-ylamino)butanoyl]-6,6-dimethyl-3-azabicyclo[3.1.0]hexane-2-carboxylate (410 mg, 1.01 mmol, 56.74% yield) as yellow oil. MS (ESI) m/z 408.2 [M+H]⁺

Step 4: (1R,2S,5S)-3-[(2S)-3,3-dimethyl-2-(thiazol-2-ylamino)butanoyl]-6,6-dimethyl-3-azabicyclo[3.1.0]hexane-2-carboxylic acid

A solution of tert-butyl (1R,2S,5S)-3-[(2S)-3,3-dimethyl-2-(thiazol-2-ylamino)butanoyl]-6,6-dimethyl-3-azabicyclo[3.1.0]hexane-2-carboxylate (410 mg, 1.01 mmol, 1 eq) in HCl/dioxane (4 M, 10 mL, 39.76 eq) was stirred at 25° C. for 16 h. Upon completion, the mixture was concentrated in the vacuum to give (1R,2S,5S)-3-[(2S)-3,3-dimethyl-2-(thiazol-2-ylamino)butanoyl]-6,6-dimethyl-3-azabicyclo[3.1.0]hexane-2-carboxylic acid (400 mg, crude) as yellow oil. MS (ESI) m/z 352.2 [M+H]⁺.

Step 5: methyl (2S)-2-[[(1R,2S,5S)-3-[(2S)-3,3-dimethyl-2-(thiazol-2-ylamino)butanoyl]-6,6-dimethyl-3-azabicyclo[3.1.0]hexane-2-carbonyl]amino]-3-[(6R)-5-oxo-4-azaspiro[2.4]heptan-6-yl]propanoate

To a solution of (1R,2S,5S)-3-[(2S)-3,3-dimethyl-2-(thiazol-2-ylamino)butanoyl]-6,6-dimethyl-3-azabicyclo[3.1.0]hexane-2-carboxylic acid (400 mg, 1.14 mmol, 1 eq) in DCM (10 mL) was added methyl (2S)-2-amino-3-[(6R)-5-oxo-4-azaspiro[2.4]heptan-6-yl]propanoate (241.56 mg, 1.14 mmol, 1 eq) then DMAP (417.12 mg, 3.41 mmol, 3 eq), followed by EDCI (436.35 mg, 2.28 mmol, 2 eq) and the mixture was stirred at 25° C. for 1 h. Upon completion, the reaction was quenched by H₂O (20 mL), and extracted with DCM (20 mL*3). The combined organic phase was washed with brine (20 mL), dried with anhydrous Na₂SO₄, filtered and concentrated in vacuum and purified by column chromatography (SiO₂, Petroleum ether:Ethyl acetate=3:7 to 1:9) to give methyl (2S)-2-[[(1R,2S,5S)-3-[(2S)-3,3-dimethyl-2-(thiazol-2-ylamino)butanoyl]-6,6-dimethyl-3-azabicyclo[3.1.0]hexane-2-carbonyl]amino]-3-[(6R)-5-oxo-4-azaspiro[2.4]heptan-6-yl]propanoate (300 mg, 549.76 umol, 48.31% yield) as yellow solid. MS (ESI) m/z 546.3 [M+H]⁺

Step 6: (1R,2S,5S)—N-[(1S)-2-amino-2-oxo-1-[[(6R)-5-oxo-4-azaspiro[2.4]heptan-6-yl]methyl]ethyl]-3-[(2S)-3,3-dimethyl-2-(thiazol-2-ylamino)butanoyl]-6,6-dimethyl-3-azabicyclo[3.1.0]hexane-2-carboxamide

A solution of methyl (2S)-2-[[(1R,2S,5S)-3-[(2S)-3,3-dimethyl-2-(thiazol-2-ylamino)butanoyl]-6,6-dimethyl-3-azabicyclo[3.1.0]hexane-2-carbonyl]amino]-3-[(6R)-5-oxo-4-azaspiro[2.4]heptan-6-yl]propanoate (280 mg, 513.11 umol, 1 eq) in NH₃/MeOH (7 M, 15 mL, 204.63 eq) was stirred at 40° C. for 16 h. Upon completion, the mixture was concentrated in the vacuum to give (1R,2S,5S)—N-[(1S)-2-amino-2-oxo-1-[[(6R)-5-oxo-4-azaspiro[2.4]heptan-6-yl]methyl]ethyl]-3-[(2S)-3,3-dimethyl-2-(thiazol-2-ylamino)butanoyl]-6,6-dimethyl-3-azabicyclo[3.1.0]hexane-2-carboxamide (250 mg, crude) as yellow solid. MS (ESI) m/z 531.3 [M+H]⁺.

Step 7: (1R,2S,5S)—N-[(1S)-1-cyano-2-[(6R)-5-oxo-4-azaspiro[2.4]heptan-6-yl]ethyl]-3-[(2S)-3,3-dimethyl-2-(thiazol-2-ylamino)butanoyl]-6,6-dimethyl-3-azabicyclo[3.1.0]hexane-2-carboxamide

A solution of (1R,2S,5S)—N-[(1S)-2-amino-2-oxo-1-[[(6R)-5-oxo-4-azaspiro[2.4]heptan-6-yl]methyl]ethyl]-3-[(2S)-3,3-dimethyl-2-(thiazol-2-ylamino)butanoyl]-6,6-dimethyl-3-azabicyclo[3.1.0]hexane-2-carboxamide (250 mg, 471.09 umol, 1 eq) in DCM (5 mL) was added burgess reagent (336.79 mg, 1.41 mmol, 3 eq) and the mixture was stirred at 25° C. for 2 h. Upon completion, the reaction was quenched by H₂O (0.5 mL), then concentrated in the vacuum and purified by prep-HPLC(column: Phenomenex C18 80*40 mm*3 um; mobile phase: [water(NH₄HCO₃)-ACN]; B %: 30%-50%, 8 min) to give (1R,2S,5S)—N-[(1S)-1-cyano-2-[(6R)-5-oxo-4-azaspiro[2.4]heptan-6-yl]ethyl]-3-[(2S)-3,3-dimethyl-2-(thiazol-2-ylamino)butanoyl]-6,6-dimethyl-3-azabicyclo[3.1.0]hexane-2-carboxamide (46.53 mg, 90.76 umol, 19.27% yield, 100% purity) as white solid. MS (ESI) m/z 513.2 [M+H]⁺.

¹H NMR (400 MHz, DMSO-d₆) δ=8.98-8.51 (m, 1H), 7.98-7.68 (m, 1H), 7.62-7.53 (m, 1H), 7.06-6.85 (m, 1H), 6.61-6.54 (m, 1H), 5.00-4.86 (m, 1H), 4.54-4.26 (m, 1H), 4.18-4.06 (m, 2H), 3.93-3.67 (m, 1H), 2.72-2.64 (m, 1H), 2.23-2.16 (m, 1H), 2.01-1.88 (m, 2H), 1.86-1.73 (m, 1H), 1.58-1.54 (m, 1H), 1.25 (d, J=7.6 Hz, 1H), 1.04 0.96 (m, 12H), 0.87-0.84 (m, 3H), 0.76-0.68 (m, 1H), 0.60-0.47 (m, 3H) Example 76: Synthesis of (1R,2S,5S)—N-[(1S)-1-cyano-2-[(6R)-5-oxo-4-azaspiro[2.4]heptan-6-yl]ethyl]-3-[(2S)-3,3-dimethyl-2-[[1-(trifluoromethyl)cyclopropanecarbonyl]amino]butanoyl]-6,6-dimethyl-3-azabicyclo[3.1.0]hexane-2-carboxamide (Compound 136)

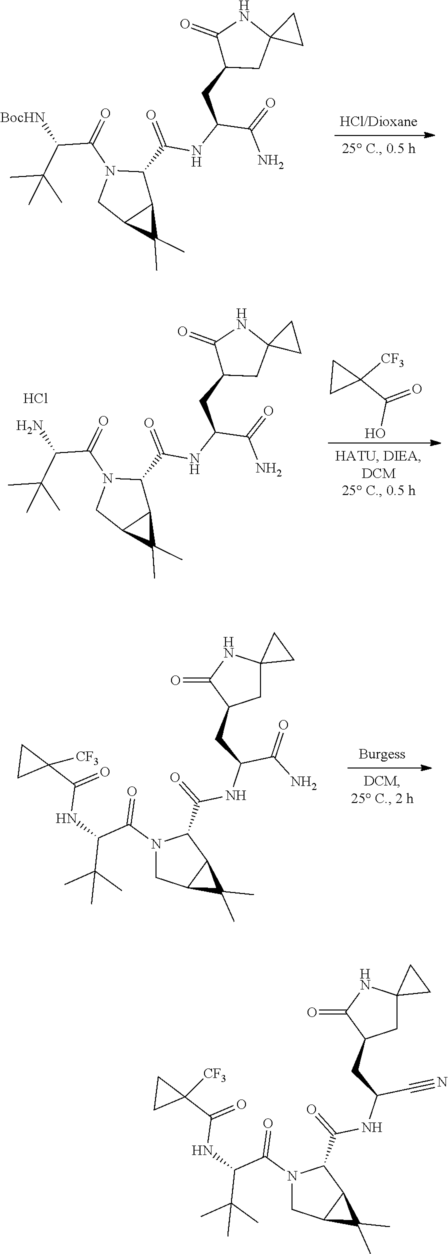

Step 1: (1R,2S,5S)-3-[(2S)-2-amino-3,3-dimethyl-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide

A mixture of tert-butyl N-[(1S)-1-[(1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl-propyl]carbamate (350 mg, 511.24 umol, 1 eq) in HCl/dioxane (4 M, 4 mL) was stirred at 25° C. for 0.5 h. Upon completion, the reaction mixture was concentrated in a vacuum to dryness to afford (1R,2S,5S)-3-[(2S)-2-amino-3,3-dimethyl-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide (240 mg, 495.84 umol, HCl) as white solid.

Step 2: (1R,2S,5S)—N-[(1S)-2-amino-2-oxo-1-[[(6R)-5-oxo-4-azaspiro[2.4]heptan-6-yl]methyl]ethyl]-3-[(2S)-3,3-dimethyl-2-[[1-(trifluoromethyl)cyclopropanecarbonyl]amino]butanoyl]-6,6-dimethyl-3-azabicyclo[3.1.0]hexane-2-carboxamide

To a mixture of (1R,2S,5S)-3-[(2S)-2-amino-3,3-dimethyl-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide (240 mg, 495.84 umol, 1 eq, HCl) and 1-(trifluoromethyl)cyclopropanecarboxylic acid (76.40 mg, 495.84 umol, 1 eq) in DCM (1 mL) was added HATU (282.80 mg, 743.75 umol, 1.5 eq) and DIEA (160.21 mg, 1.24 mmol, 215.91 uL, 2.5 eq) in turn. After the addition, the resulting mixture was stirred at 25° C. for 0.5 h. Upon completion, the reaction mixture was quenched 2 mL 20% citric acid (aq.). After that, the organic layer was separated, dried over Na₂SO₄, filtered and concentrated in vacuum to afford a residue. The residue was purified by prep-TLC (SiO₂, Ethyl acetate:Methanol=10:1) to give (1R,2S,5S)—N-[(1S)-2-amino-2-oxo-1-[[(6R)-5-oxo-4-azaspiro[2.4]heptan-6-yl]methyl]ethyl]-3-[(2S)-3,3-dimethyl-2-[[1-(trifluoromethyl)cyclopropanecarbonyl]amino]butanoyl]-6,6-dimethyl-3-azabicyclo[3.1.0]hexane-2-carboxamide (280 mg, 213.67 umol, 43.09% yield, 44.539% purity) as white solid. MS (ESI) m/z 584.4 [M+H]⁺

Step 3: (1R,2S,5S)—N-[(1S)-1-cyano-2-[(6R)-5-oxo-4-azaspiro[2.4]heptan-6-yl]ethyl]-3-[(2S)-3,3-dimethyl-2-[[1-(trifluoromethyl)cyclopropanecarbonyl]amino]butanoyl]-6,6-dimethyl-3-azabicyclo[3.1.0]hexane-2-carboxamide

To a mixture of (1R,2S,5S)—N-[(1S)-2-amino-2-oxo-1-[[(6R)-5-oxo-4-azaspiro[2.4]heptan-6-yl]methyl]ethyl]-3-[(2S)-3,3-dimethyl-2-[[1-(trifluoromethyl)cyclopropanecarbonyl]amino]butanoyl]-6,6-dimethyl-3-azabicyclo[3.1.0]hexane-2-carboxamide (280 mg, 479.75 umol, 1 eq) in DCM (4 mL) was added burgess reagent (251.52 mg, 1.06 mmol, 2.2 eq). After the addition, the resulting mixture was stirred at 25° C. for 2 h. Upon completion, the reacting mixture was quenched by water (0.5 mL), blowed by N₂ to dryness and diluted by DMF (1 mL) to afford a residue. The residue was purified by prep-HPLC (column: Waters Xbridge BEH C18 100*30 mm*10 um; mobile phase: [water(NH₄HCO₃)-ACN]; B %: 40%-60%, 8 min) to give the product (1R,2S,5S)—N-[(1S)-1-cyano-2-[(6R)-5-oxo-4-azaspiro[2.4]heptan-6-yl]ethyl]-3-[(2S)-3,3-dimethyl-2-[[1-(trifluoromethyl)cyclopropanecarbonyl]amino]butanoyl]-6,6-dimethyl-3-azabicyclo[3.1.0]hexane-2-carboxamide (21.14 mg, 35.46 umol, 7.39% yield, 94.888% purity) as a white solid. MS (ESI) m/z 566.2 [M+H]⁺

¹H NMR (400 MHz, DMSO-d₆) δ=9.01 (d, J=8.4 Hz, 1H), 7.75 (s, 1H), 7.36 (br d, J=8.6 Hz, 1H), 4.97-4.88 (m, 1H), 4.43 (br d, J=8.9 Hz, 1H), 4.15 (s, 1H), 3.86 (br dd, J=5.4, 10.4 Hz, 1H), 3.70 (br d, J=10.0 Hz, 1H), 2.62-2.62 (m, 1H), 2.20 (br dd, J=5.3, 8.1 Hz, 1H), 2.01-1.91 (m, 2H), 1.85-1.75 (m, 1H), 1.54 (dd, J=5.3, 7.3 Hz, 1H), 1.31-1.20 (m, 4H), 1.15 (br s, 1H), 1.02 (s, 3H), 0.93 (s, 9H), 0.81 (s, 3H), 0.77-0.69 (m, 1H), 0.65-0.46 (m, 3H).

Example 77: Synthesis of (1R,2S,5S)—N-[(1S)-1-cyano-2-[(6R)-5-oxo-4-azaspiro[2.4]heptan-6-yl]ethyl]-3-[(2S)-2-(2,2-dimethylpropanoylamino)-3,3-dimethyl-butanoyl]-6,6-dimethyl-3-azabicyclo[3.1.0]hexane-2-carboxamide (Compound 135)

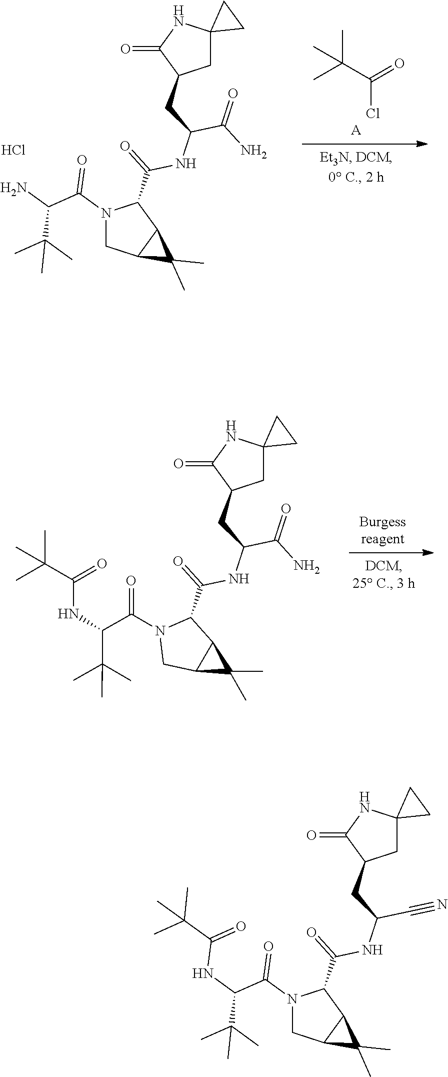

Step 1: (1R,2S,5S)—N-[(1S)-2-amino-2-oxo-1-[[(6R)-5-oxo-4-azaspiro[2.4]heptan-6-yl]methyl]ethyl]-3-[(2S)-2-(2,2-dimethylpropanoylamino)-3,3-dimethyl-butanoyl]-6,6-dimethyl-3-azabicyclo[3.1.0]hexane-2-carboxamide

To a solution of (1R,2S,5S)-3-[(2S)-2-amino-3,3-dimethyl-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]exane 2-carboxamide (330 mg, 545.42 umol, 80% purity, 1 eq, HCl) in DCM (15 mL) was added Et₃N (165.57 mg, 1.64 mmol, 227.75 uL, 3 eq), and then 2,2-dimethylpropanoyl chloride (98.65 mg, 818.13 umol, 100.66 uL, 1.5 eq) at 0° C. The resulting mixture was stirred at 0° C. for 2 h. Upon completion, the reaction mixture was quenched by addition H₂O 60 mL at 0° C., and then extracted with DCM 90 mL (30 mL*3). The combined organic layers were washed with brine 50 mL, dried over Na₂SO₄, filtered and concentrated under reduced pressure to give (1R,2S,5S)—N-[(1S)-2-amino-2-oxo-1-[[(6R)-5-oxo-4-azaspiro[2.4]heptan-6-yl]methyl]ethyl]-3-[(2S)-2-(2,2-dimethylpropanoylamino)-3,3-dimethyl-butanoyl]-6,6-dimethyl-3-azabicyclo[3.1.0]hexane-2-carboxamide (320 mg, crude) as a yellow oil.

Step 2: (1R,2S,5S)—N-[(1S)-1-cyano-2-[(6R)-5-oxo-4-azaspiro[2.4]heptan-6-yl]ethyl]-3-[(2S)-2-(2,2-dimethylpropanoylamino)-3,3-dimethyl-butanoyl]-6,6-dimethyl-3-azabicyclo[3.1.0]hexane-2-carboxamide

To a solution of (1R,2S,5S)—N-[(1S)-2-amino-2-oxo-1-[[(6R)-5-oxo-4-azaspiro[2.4]heptan-6-yl]methyl]ethyl]-3-[(2S)-2-(2,2-dimethylpropanoylamino)-3,3-dimethyl-butanoyl]-6,6-dimethyl-3-azabicyclo[3.1.0]hexane-2-carboxamide (300 mg, 1 eq) in DCM (15 mL) was added burgess reagent (564.74 mg, 2.37 mmol, 6 eq). The mixture was stirred at 25° C. for 3 h. Upon completion, water (1.5 mL) was added to the solution, then dried by blowing N₂. The residue was purified by prep-HPLC (column: Waters Xbridge Prep OBD C18 150*40 mm*10 um; mobile phase: [water(NH₄HCO₃)-ACN]; B %: 35%-65%, 8 min) to give (1R,2S,5S)—N-[(1S)-1-cyano-2-[(6R)-5-oxo-4-azaspiro[2.4]heptan-6-yl]ethyl]-3-[(2S)-2-(2,2-dimethylpropanoylamino)-3,3-dimethyl-butanoyl]-6,6-dimethyl-3-azabicyclo[3.1.0]hexane-2-carboxamide (43.46 mg, 84.61 umol, 21.42% yield, 100% purity) as a white solid. MS (ESI) m/z 514.3 [M+H]⁺.

¹H NMR (400 MHz, DMSO-d₆) δ=8.96 (d, J=8.5 Hz, 1H), 7.74 (s, 1H), 6.85 (d, J=9.3 Hz, 1H), 4.98-4.88 (m, 1H), 4.43 (d, J=9.3 Hz, 1H), 4.13 (s, 1H), 3.85 (dd, J=5.3, 10.3 Hz, 1H), 3.72 (d, J=10.4 Hz, 1H), 2.67-2.61 (m, 1H), 2.23-2.15 (m, 1H), 2.01 1.91 (m, 2H), 1.81 (ddd, J=5.3, 10.6, 13.4 Hz, 1H), 1.53 (dd, J=5.6, 7.2 Hz, 1H), 1.28 (d, J=7.6 Hz, 1H), 1.13-1.04 (m, 9H), 1.02 (s, 3H), 0.92 (s, 9H), 0.81 (s, 3H), 0.76-0.70 (m, 1H), 0.59-0.50 (m, 3H).

Example 78: Synthesis of tetrahydrofuran-3-yl N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl-propyl]carbamate (Compound 134)

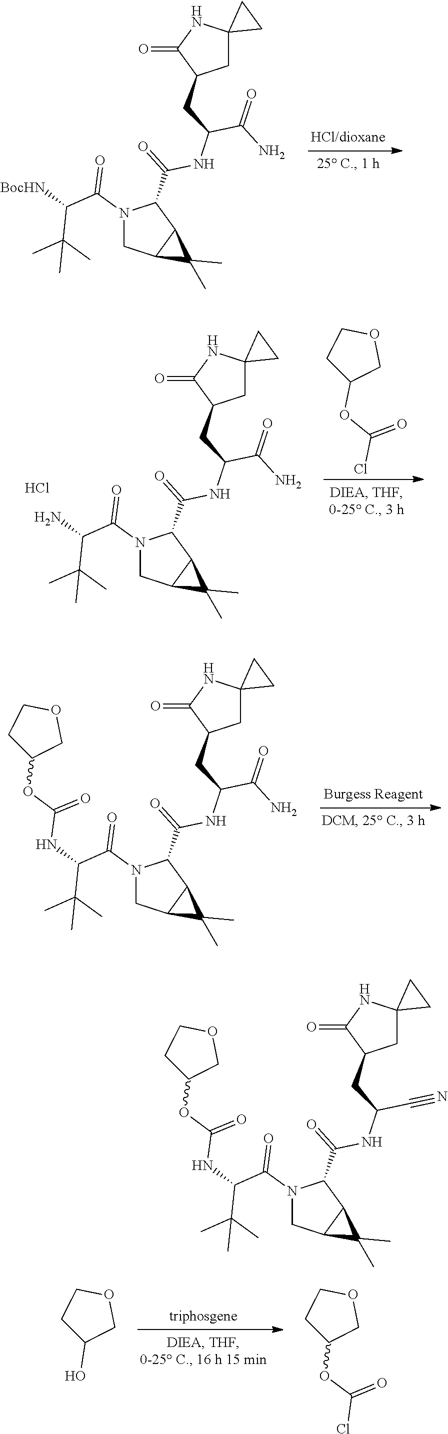

Step 1: tetrahydrofuran-3-yl carbonochloridate

To a mixture of tetrahydrofuran-3-ol (200 mg, 2.27 mmol, 183.49 uL, 1 eq) in THE (3 mL) was added DIEA (586.77 mg, 4.54 mmol, 790.79 uL, 2 eq) at 0° C. bis(trichloromethyl) carbonate (916.13 mg, 3.09 mmol, 1.36 eq) in THE (3 mL) was added at 0° C. The mixture was stirred at 0° C. for 15 min and then heated to 25° C. and stirred for 16 h. Upon completion, the mixture was concentrated in vacuum to obtained tetrahydrofuran-3-yl carbonochloridate (400 mg, crude) as a yellow oil.

Step 2: (1S,3aR,6aS)-2-(tert-butoxycarbonyl)octahydrocyclopenta[c]pyrrole-1-carboxylic acid

A solution of tert-butyl N-[1-[(1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl-propyl]carbamate (200 mg, 365.17 umol, 1 eq) in HCl/dioxane (3 mL, 4 M) was stirred at 25° C. for 1 h. Upon completion, the mixture was concentrated in vacuum to obtained (1R,2S,5S)-3-[(2S)-2-amino-3,3-dimethyl-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide (170 mg, crude, HCl) as a yellow solid.

Step 3: (S)-methyl 2-amino-3-((R)-5-oxo-4-azaspiro[2.4]heptan-6-yl)propanoate

To a mixture of (1R,2S,5S)-3-[(2S)-2-amino-3,3-dimethyl-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide (170 mg, 351.22 umol, 1 eq, HCl) in THE (6 mL) was added DIEA (136.17 mg, 1.05 mmol, 183.52 uL, 3 eq) and the solution of tetrahydrofuran-3-yl carbonochloridate (211.52 mg, 702.44 umol, 50% purity, 2 eq) in THE (6 mL) at 0° C. The mixture was stirred at 25° C. for 3 h. Upon completion, the mixture was concentrated in vacuum. The crude product was purified by prep-HPLC (column: Waters Xbridge Prep OBD C18 150*40 mm*10 um; mobile phase: [water (NH₄HCO₃)-ACN]; B %: 20%-40%, 8 min) to afford tetrahydrofuran-3-yl N-[(1S)-1-[(1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl-propyl]carbamate Isomer 1 (70 mg, 112.17 umol, 31.94% yield, 90% purity) as a white solid.

Tetrahydrofuran-3-yl N-[(1S)-1-[(1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl-propyl]carbamate Isomer 2 was obtained (70 mg, 112.17 umol, 31.94% yield, 90% purity) as a white solid.

Step 4: tetrahydrofuran-3-yl N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl propyl]carbamate Isomer 1

To a solution of tetrahydrofuran-3-yl N-[(1S)-1-[(1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl-propyl]carbamate Isomer 1 (70 mg, 124.63 umol, 1 eq) in DCM (2 mL) was added burgess reagent (118.80 mg, 498.51 umol, 4 eq), the mixture was stirred at 25° C. for 3 h. Upon completion, the mixture was quenched by water (0.5 mL) and was dried by blowing N₂ and was purified by prep-HPLC (column: Waters Xbridge BEH C18 100*30 mm*10 um; mobile phase: [water (NH₄HCO₃)-ACN]; B %: 30%-60%, 8 min) to obtained tetrahydrofuran-3-yl N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl-propyl]carbamate Isomer 1 (25 mg, 45.99 umol, 36.90% yield, 100% purity) as a white solid. MS (ESI) m/z 544.2 [M+H]⁺

¹H NMR (400 MHz, DMSO-d₆) δ=8.99 (d, J=8.5 Hz, 1H), 7.73 (s, 1H), 7.23 (d, J=8.8 Hz, 1H), 5.10-4.99 (m, 1H), 4.97-4.86 (m, 1H), 4.15 (s, 1H), 4.02 (d, J=8.7 Hz, 1H), 3.88-3.78 (m, 2H), 3.76-3.60 (m, 4H), 2.71-2.60 (m, 1H), 2.19 (ddd, J=4.6, 10.8, 13.6 Hz, 1H), 2.13-2.03 (m, 1H), 2.01-1.87 (m, 2H), 1.86-1.71 (m, 2H), 1.54 (br dd, J=4.8, 7.2 Hz, 1H), 1.27 (d, J=7.6 Hz, 1H), 1.07-1.00 (m, 3H), 0.97-0.90 (m, 9H), 0.88 0.85 (m, 3H), 0.76-0.69 (m, 1H), 0.59-0.50 (m, 3H).

tetrahydrofuran-3-yl N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl-propyl]carbamate Isomer 2

To a solution of tetrahydrofuran-3-yl N-[(1S)-1-[(1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl-propyl]carbamate Isomer 2 (70 mg, 124.63 umol, 1 eq) in DCM (2 mL) was added burgess reagent (118.80 mg, 498.51 umol, 4 eq), the mixture was stirred at 25° C. for 3 h. Upon completion, the mixture was quenched by water (0.5 mL) and was dried by blowing N₂. The crude product was purified by prep-HPLC (column: Waters Xbridge BEH C18 100*30 mm*10 um; mobile phase: [water (NH₄HCO₃)-ACN]; B %: 30%-60%, 8 min) to obtained tetrahydrofuran-3-yl N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl-propyl]carbamate Isomer 2 (25 mg, 45.99 umol, 36.90% yield, 100% purity) as a white solid. MS (ESI) m/z 544.2 [M+H]⁺

¹H NMR (400 MHz, DMSO-d₆) δ=9.05-8.92 (m, 1H), 7.73 (s, 1H), 7.28-7.17 (m, 1H), 5.05 (br s, 1H), 4.92 (ddd, J=5.4, 8.6, 10.6 Hz, 1H), 4.16 (s, 1H), 4.03 (br d, J=8.8 Hz, 1H), 3.78-3.64 (m, 5H), 3.53 (br d, J=10.2 Hz, 1H), 2.70-2.59 (m, 1H), 2.23-2.15 (m, 1H), 2.12-2.02 (m, 1H), 2.01-1.90 (m, 2H), 1.88-1.75 (m, 2H), 1.57-1.50 (m, 1H), 1.27 (br d, J=7.6 Hz, 1H), 1.03 (s, 3H), 0.93 (s, 9H), 0.88 (s, 3H), 0.76-0.69 (m, 1H), 0.62 0.46 (m, 3H).

Example 79: Synthesis of 2-methoxyethyl ((S)-1-((1R,2S,5S)-2-(((S)-1-cyano-2-((R)-5-oxo-4-azaspiro[2.4]heptan-6-yl)ethyl)carbamoyl)-6,6-dimethyl-3-azabicyclo[3.1.0]hexan-3-yl)-3,3-dimethyl-1-oxobutan-2-yl)carbamate (Compound 133)

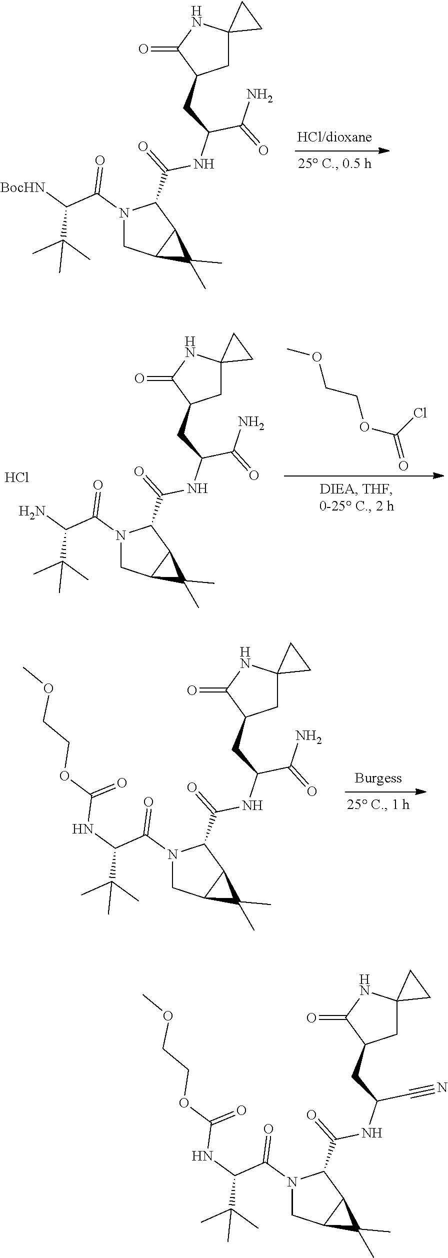

Step 1: (1R,2S,5S)-3-[(2S)-2-amino-3,3-dimethyl-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide

A solution of tert-butyl N-[(1S)-1-[(1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl-propyl]carbamate (300.00 mg, 547.76 umol, 1 eq) in HCl/dioxane (4 M, 136.94 uL, 1 eq) was stirred at 25° C. for 1 h. Upon completion, the reaction mixture was concentrated under reduced pressure to give (1R,2S,5S)-3-[(2S)-2-amino-3,3-dimethyl-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide (265 mg, crude, HCl) as a yellow solid.

Step 2: 2-methoxyethyl N-[(1S)-1-[(1R,2S,5S)-2-[((1S)-2-amino-2-oxo-1-[[(6R)-5-oxo-4-azaspiro[2.4]heptan-6-yl/methyl/ethyl]carbamoyl]-6,6-dimethyl-3-azabicyclo[3.1.0]hexane-3-carbonyl]-2,2-dimethyl propyl/carbamate

A solution of (1R,2S,5S)-3-[(2S)-2-amino-3,3-dimethyl-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide (265 mg, 592.09 umol, 1 eq) in THE (5 mL), was added with DIEA (229.57 mg, 1.78 mmol, 309.39 uL, 3 eq), and then 2-methoxyethyl carbonochloridate (123.05 mg, 888.13 umol, 103.40 uL, 1.5 eq) in THE (1 mL) was added at 0° C. The mixture was stirred at 25° C. for 2 h. Upon completion, the reaction mixture was poured into H₂O 30 mL at 25° C., and then extracted with EtOAc (30 mL*3). The combined organic layers were washed with brine 30 mL, dried over Na₂SO₄, filtered and concentrated under reduced pressure to give a residue. The residue was purified by column chromatography (SiO₂, DCM:MeOH=100:0 to 95:5) to give 2-methoxyethyl N-[(1S)-1 [(1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl-propyl]carbamate (117 mg, 202.22 umol, 34.15% yield, 95% purity) as a yellow solid. MS (ESI) m/z 550.32 [M+H]⁺.

Step 3: 2-methoxyethyl N-[(1S)-1-[(1R,2S,5S)-2-[[(1S)-1-cyano-2-[(6R)-5-oxo-4-azaspiro[2.4]heptan-6-yl/ethyl]carbamoyl]-6,6-dimethyl-3-azabicyclo[3.1.0]hexane-3-carbonyl]-2,2-dimethyl propyl/carbamate

A solution of 2-methoxyethyl N-[(1S)-1-[(1R,2S,5S)-2-[[(1S)-2-amino-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carbonyl]-2,2-dimethyl-propyl]carbamate (117 mg, 212.86 umol, 1 eq) in DCM (2 mL) was added burgess reagent (202.90 mg, 851.44 umol, 4 eq). The mixture was stirred at 25° C. for 1 h. Upon completion, the reaction mixture was poured into H₂O 30 mL at 25° C., and then extracted with DCM (30 mL*3). The combined organic layers were washed with brine 30 mL, dried over Na₂SO₄, filtered and concentrated under reduced pressure to give a residue. The residue was purified by prep-HPLC (column: Waters Xbridge Prep OBD C18 150*40 mm*10 um; mobile phase: [water(NH₄HCO₃)-ACN]; B %: 25%-45%, 8 min) to afford 2-methoxyethyl N-[(1S)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2,2-dimethyl-propyl]carbamate (25.18 mg, 47.36 umol, 22.25% yield, 100% purity) as a white solid. MS (ESI) m/z 532.31 [M+H]⁺.

¹H NMR (400 MHz, METHANOL-d4) δ=5.01 (dd, J=5.1, 10.8 Hz, 1H), 4.26 (s, 1H), 4.21 (s, 1H), 4.17-4.11 (m, 2H), 4.02-3.89 (m, 2H), 3.60-3.53 (m, 2H), 3.37 3.33 (m, 3H), 2.88 (d, J=4.6, 10.2 Hz, 1H), 2.43-2.33 (m, 1H), 2.22-2.13 (m, 1H), 2.11 2.01 (m, 1H), 2.00-1.90 (m, 1H), 1.62 (dd, J=5.3, 7.4 Hz, 1H), 1.38 (d, J=7.6 Hz, 1H), 1.11-1.07 (m, 3H), 1.02 (s, 9H), 0.96 (s, 3H), 0.88-0.82 (m, 1H), 0.75-0.59 (m, 3H)

Example 80: Synthesis of 2,2,2-trifluoroethyl ((S)-1-((1R,2S,5S)-2-(((S)-1-cyano-2-((R)-5-oxo-4-azaspiro[2.4]heptan-6-yl)ethyl)carbamoyl)-6,6-dimethyl-3-azabicyclo[3.1.0]hexan-3-yl)-3,3-dimethyl-1-oxobutan-2-yl)carbamate (Compound 132)

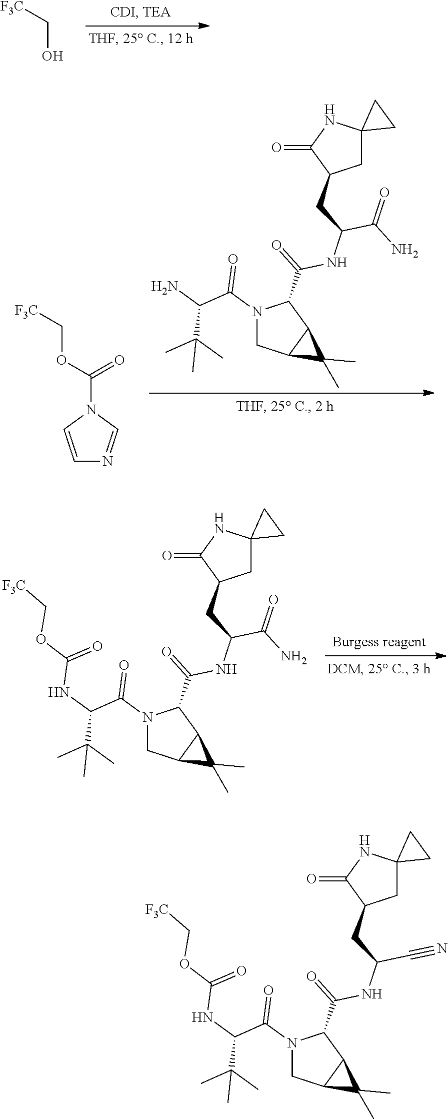

Step 1: 2,2,2-trifluoroethyl 1H-imidazole-1-carboxylate

To a solution of 2,2,2-trifluoroethanol (100 mg, 999.60 umol, 71.94 uL, 1 eq) in THE (1 mL) was added CDI (178.29 mg, 1.10 mmol, 1.1 eq) and TEA (303.45 mg, 3.00 mmol, 417.40 uL, 3 eq), then the mixture was stirred at 25° C. for 12 h. Upon completion, the crude product was used as a solution. 2,2,2-trifluoroethyl 1H-imidazole-1-carboxylate (190 mg, 978.82 umol, 97.92% yield) as a colourless liquid in THF.

Step 2: 2,2,2-trifluoroethyl ((S)-1-((1R,2S,5S)-2-(((S)-1-amino-1-oxo-3-((R)-5-oxo-4-azaspiro[2.4]heptan-6-yl)propan-2-yl)carbamoyl)-6,6-dimethyl-3-azabicyclo(3.1.0]hexan-3-yl)-3,3-dimethyl-1-oxobutan-2-yl)carbamate

To a solution of (1R,2S,5S)-N—((S)-1-amino-1-oxo-3-((R)-5-oxo-4-azaspiro[2.4]heptan-6-yl)propan-2-yl)-3-((S)-2-amino-3,3-dimethylbutanoyl)-6,6-dimethyl-3-azabicyclo[3.1.0]hexane-2-carboxamide (178 mg, 367.75 umol, 1 eq, HCl) in THE (2 mL) was added 2,2,2-trifluoroethyl 1H-imidazole-1-carboxylate (107.08 mg, 551.62 umol, 1.5 eq) at 25° C., the mixture was stirred at 25° C. for 1 h. LCMS showed most starting material was remained, then the mixture was stirred for another 1 h. Upon completion, the reaction mixture was quenched by H₂O (5 mL), extracted with EtOAc (5 mL*2). The combined organic layers were washed with brine (5 mL*2), dried over Na₂SO₄, filtered and concentrated under reduced pressure to give a residue. The residue was purified by column chromatography (SiO₂, Petroleum ether:Ethyl acetate=1:1 to EA:MeOH=5:1) to give 2,2,2-trifluoroethyl ((S)-1-((1R,2S,5S)-2-(((S)-1-amino-1-oxo-3-((R)-5-oxo-4-azaspiro[2.4]heptan-6-yl)propan-2-yl)carbamoyl)-6,6-dimethyl-3-azabicyclo[3.1.0]hexan-3-yl)-3,3-dimethyl-1-oxobutan-2-yl)carbamate (120 mg, 209.20 umol, 56.89% yield) as a white solid. MS (ESI) m/z 574.3[M+H]⁺.

Step 3: 2,2,2-trifluoroethyl ((S)-1-((1R,2S,5S)-2-(((S)-1-cyano-2-((R)-5-oxo-4-azaspiro[2.4]heptan-6-yl)ethyl)carbamoyl)-6,6-dimethyl-3-azabicyclo[3.1.0]hexan-3-yl)-3,3-dimethyl-1-oxobutan-2-yl)carbamate

To a solution of 2,2,2-trifluoroethyl ((S)-1-((1R,2S,5S)-2-(((S)-1-amino-1-oxo-3-((R)-5-oxo-4-azaspiro[2.4]heptan-6-yl)propan-2-yl)carbamoyl)-6,6-dimethyl-3-azabicyclo[3.1.0]hexan-3-yl)-3,3-dimethyl-1-oxobutan-2-yl)carbamate (100 mg, 174.34 umol, 1 eq) in DCM (3 mL) was added burgess reagent (124.64 mg, 523.01 umol, 3 eq), the mixture was stirred at 25° C. for 2 h. LCMS showed most starting material was remained, then was added methoxycarbonyl-(triethylammonio)sulfonyl-azanide (83.09 mg, 348.67 umol, 2 eq) and was stirred for another 1 h. Upon completion, the resulting solution was quenched with H₂O (0.5 mL), then was concentrated in vacuum (25° C.). The residue was purified by prep-HPLC column: Waters Xbridge Prep OBD C18 150*40 mm*10 um; mobile phase: [water(NH₄HCO₃)-ACN]; B %: 25%-55%, 8 min to give 2,2,2-trifluoroethyl ((S)-1-((1R,2S,5S)-2-(((S)-1-cyano-2-((R)-5-oxo-4-azaspiro[2.4]heptan-6-yl)ethyl)carbamoyl)-6,6-dimethyl-3-azabicyclo[3.1.0]hexan-3-yl)-3,3-dimethyl-1-oxobutan-2-yl)carbamate (27.27 mg, 49.08 umol, 28.15% yield, 100% purity) as a white solid. MS (ESI) m/z 556.3 [M+H]⁺.

¹H NMR (400 MHz, DMSO-d₆) δ=9.20-8.80 (m, 1H), 7.85-7.79 (m, 1H), 7.76-7.72 (m, 1H), 4.98-4.89 (m, 1H), 4.73-4.50 (m, 2H), 4.20-4.13 (m, 1H), 4.06 (br d, J=8.7 Hz, 1H), 3.91-3.81 (m, 1H), 3.79-3.70 (m, 1H), 2.71-2.58 (m, 1H), 2.27-2.15 (m, 1H), 2.03-1.89 (m, 2H), 1.86-1.72 (m, 1H), 1.63-1.51 (m, 1H), 1.36-1.22 (m, 1H), 1.02 (s, 3H), 0.94 (s, 9H), 0.88-0.83 (m, 3H), 0.77 (br s, 1H), 0.61-0.46 (m, 3H)

Example 81: Synthesis of (1R⁵S)-3-[(2S,3R)-3-tert-butoxy-2-[(2,2,2-trifluoroacetyl)amino]butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (Compound 137)

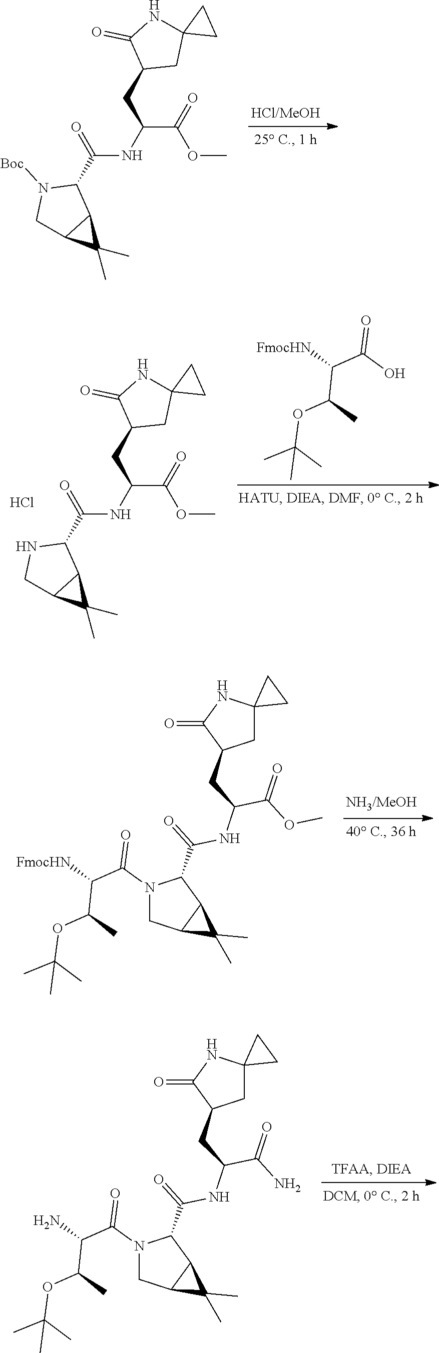

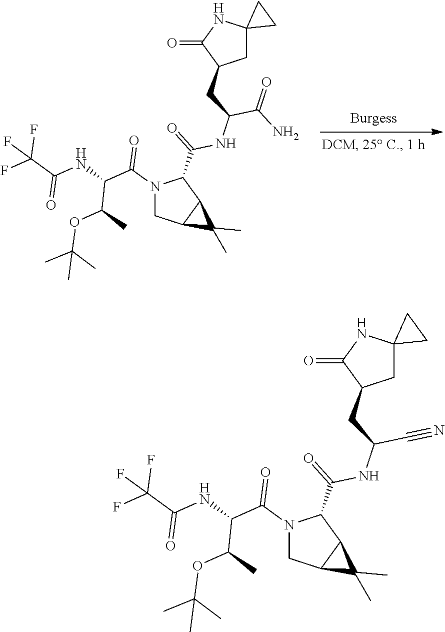 [SMILES: C[C@@H](OC(C)(C)C)[C@H](NC(=O)C(F)(F)F)C(=O)N1C[C@H]2[C@@H]([C@H]1C(=O)N[C@@H](C[C@@H]1CC3(CC3)NC1=O)C(N)=O)C2(C)C ; C[C@@H](OC(C)(C)C)[C@H](NC(=O)C(F)(F)F)C(=O)N1C[C@H]2[C@@H]([C@H]1C(=O)N[C@H](C#N)C[C@@H]1CC3(CC3)NC1=O)C2(C)C]

Step 1: methyl (2S)-2-[[(1R,2S,5S)-6,6-dimethyl-3-azabicyclo[3.1.0]hexane-2-carbonyl]amino]-3-[(6R)-5-oxo-4-azaspiro[2.4]heptan-6-yl]propanoate

A mixture of tert-butyl (1R,2S,5S)-2-[[(1S)-2-methoxy-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carboxylate (0.25 g, 556.13 umol, 1 eq) in HCl/MeOH (4 M, 8 mL, 57.54 eq) was stirred at 25° C. for 1 h. Upon completion, the reaction mixture was concentrated under reduced pressure to give methyl (2S)-2-[[(1R,2S,5S)-6,6-dimethyl-3-azabicyclo[3.1.0]hexane-2-carbonyl]amino]-3-[(6R)-5-oxo-4-azaspiro[2.4]heptan-6-yl]propanoate (200 mg, crude, HCl) as a white solid.

Step 2: methyl (2S)-2-[[(1R,2S,5S)-3-[(2S,3R)-3-tert-butoxy-2-(9H-fluoren-9-ylmethoxycarbonylamino)butanoyl]-6,6-dimethyl-3-azabicyclo[3.1.0]hexane-2-carbonyl]amino]-3-[(6R)-5-oxo-4-azaspiro[2.4]heptan-6-yl]propanoate

To a solution of methyl (2S)-2-[[(1R,2S,5S)-6,6-dimethyl-3-azabicyclo[3.1.0]hexane-2-carbonyl]amino]-3-[(6R)-5-oxo-4-azaspiro[2.4]heptan-6-yl]propanoate (200 mg, 518.29 umol, 1 eq, HCl) and (2S,3R)-3-tert-butoxy-2-(9H-fluoren-9-ylmethoxycarbonylamino)butanoic acid (185.40 mg, 466.46 umol, 0.9 eq) in DMF (12 mL) was added HATU (394.14 mg, 1.04 mmol, 2 eq), then DIEA (200.95 mg, 1.55 mmol, 270.82 uL, 3 eq) was added. The mixture was stirred at 0° C. for 2 h. Upon completion, the reaction mixture was diluted with water (15 mL) and extracted with DCM (5 mL*3). The combined organic layers were dried over Na₂SO₄, filtered and concentrated under reduced pressure to give a residue. The residue was purified by prep-TLC (SiO₂, petroleum ether:Ethyl acetate=1:2) to afford methyl (2S)-2-[[(1R,2S,5S)-3-[(2S,3R)-3-tert-butoxy-2-(9H-fluoren-9-ylmethoxycarbonylamino)butanoyl]-6,6-dimethyl-3-azabicyclo[3.1.0]hexane-2-carbonyl]amino]-3-[(6R)-5-oxo-4-azaspiro[2.4]heptan-6-yl]propanoate (238 mg, 326.53 umol, 63.00% yield) as a white solid.

Step 3: (1R,2S,5S)-3-[(2S,3R)-2-amino-3-tert-butoxy-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide

A solution of methyl (2S)-2-[[(1R,2S,5S)-3-[(2S,3R)-3-tert-butoxy-2-(9H-fluoren-9-ylmethoxycarbonylamino)butanoyl]-6,6-dimethyl-3-azabicyclo[3.1.0]hexane-2-carbonyl]amino]-3-[(6R)-5-oxo-4-azaspiro[2.4]heptan-6-yl]propanoate (230 mg, 315.56 umol, 1 eq) in NH₃/MeOH (7 M, 9.99 mL, 221.65 eq) was stirred at 40° C. for 36 h. Upon completion, the reaction mixture was concentrated under reduced pressure to give (1R,2S,5S)-3-[(2S,3R)-2-amino-3-tert-butoxy-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide (0.1 g, 193.24 umol, 61.24% yield, 95% purity) as a white solid.

Step 4: (1R,2S,5S)—N-[(1S)-2-amino-2-oxo-1-[[(6R)-5-oxo-4-azaspiro[2.4]heptan-6-yl]methyl]ethyl]-3-[(2S,3R)-3-tert-butoxy-2-[(2,2,2-trifluoroacetyl)amino]butanoyl]-6,6-dimethyl-3-azabicyclo[3.1.0]hexane-2-carboxamide

A mixture of (1R,2S,5S)-3-[(2S,3R)-2-amino-3-tert-butoxy-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide (100 mg, 203.41 umol, 1 eq) in DCM (2 mL) was added DIPEA (78.87 mg, 610.23 umol, 106.29 uL, 3 eq), then TFAA (85.44 mg, 406.82 umol, 56.59 uL, 2 eq) in DCM (0.1 mL) was drop-wise for 0.1 h, the mixture was stirred at 0° C. for 1.9 h. Upon completion, the reaction mixture was poured into saturated NaHCO₃(8 mL) and extracted with DCM (4 mL*3). The combined organic layers was filtered and concentrated under reduced pressure to give (1R,2S,5S)—N-[(1S)-2-amino-2-oxo-1-[[(6R)-5-oxo-4-azaspiro[2.4]heptan-6-yl]methyl]ethyl]-3-[(2S,3R)-3-tert-butoxy-2-[(2,2,2-trifluoroacetyl)amino]butanoyl]-6,6-dimethyl-3-azabicyclo[3.1.0]hexane-2-carboxamide (120 mg, crude) as a white solid.

Step 5: (1R,2S,5S)-3-[(2S,3R)-3-tert-butoxy-2-[(2,2,2-trifluoroacetyl)amino]butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide

To a solution of (1R,2S,5S)—N-[(1S)-2-amino-2-oxo-1-[[(6R)-5-oxo-4-azaspiro[2.4]heptan-6-yl]methyl]ethyl]-3-[(2S,3R)-3-tert-butoxy-2-[(2,2,2-trifluoroacetyl)amino]butanoyl]-6,6-dimethyl-3-azabicyclo[3.1.0]hexane-2-carboxamide (120 mg, 204.21 umol, 1 eq) in DCM (1 mL) was added BURGESS REAGENT (48.67 mg, 204.21 umol, 1 eq), the mixture was stirred at 25° C. for 1 h. Upon completion, water (1 mL) was added to the solution, then dried by blowing N₂. The crude product was purified by prep-HPLC (column: Phenomenex C18 80*40 mm*3 um; mobile phase: [water(NH₄HCO₃)-ACN]; B %: 25%-55%, 8 min) to give (1R,2S,5S)-3-[(2S,3R)-3-tert-butoxy-2-[(2,2,2-trifluoroacetyl)amino]butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (0.02 g, 34.65 umol, 16.97% yield, 98.7% purity) as a white solid. MS (ESI) m/z 569.3 [M+H]⁺.

¹H NMR (400 MHz, DMSO-d₆) δ=9.79-9.63 (m, 1H), 8.99 (d, J=8.4 Hz, 1H), 7.78 (s, 1H), 4.94 (ddd, J=5.4, 8.3, 10.4 Hz, 1H), 4.40 (br t, J=7.1 Hz, 1H), 4.13 (s, 1H), 3.97 (dd, J=5.3, 10.2 Hz, 1H), 3.90 (dd, J=6.2, 8.1 Hz, 1H), 3.68-3.63 (m, 1H), 2.67-2.59 (m, 1H), 2.26-2.10 (m, 1H), 2.04-1.89 (m, 2H), 1.83 (ddd, J=5.6, 10.5, 13.7 Hz, 1H), 1.57 (dd, J=5.6, 7.3 Hz, 1H), 1.33 (d, J=7.6 Hz, 1H), 1.17-1.03 (m, 15H), 0.84 (s, 3H), 0.80 0.72 (m, 1H), 0.64-0.51 (m, 3H).

Example 82: Synthesis of (1R,2S,5S)-3-[(2S,3S)-3-tert-butoxy-2-[(2,2,2-trifluoroacetyl)amino]butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (Compound 138)

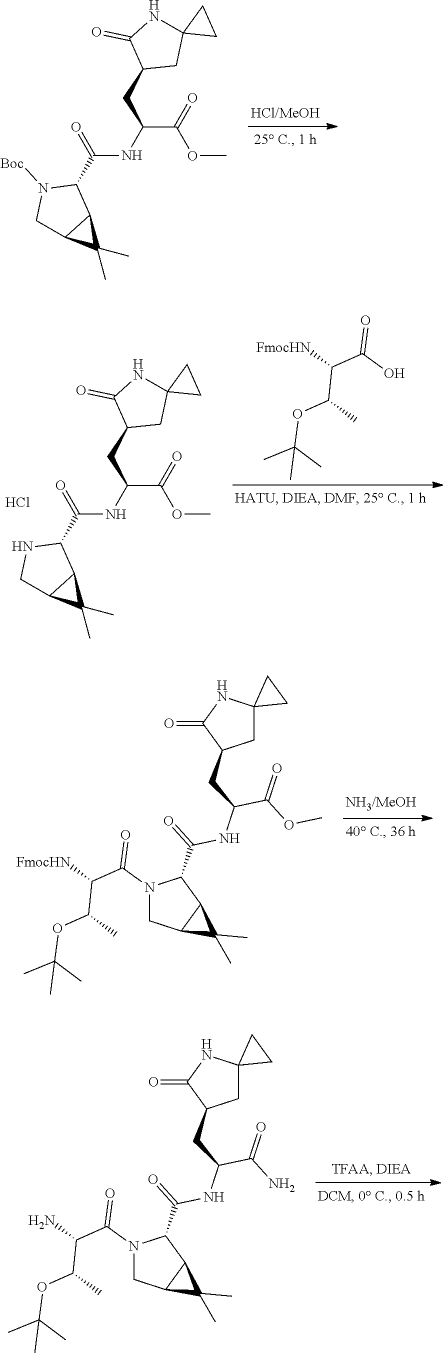

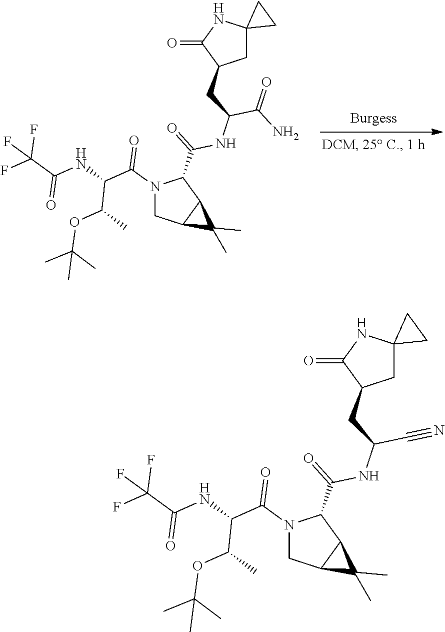 [SMILES: C[C@H](OC(C)(C)C)[C@H](NC(=O)C(F)(F)F)C(=O)N1C[C@H]2[C@@H]([C@H]1C(=O)N[C@@H](C[C@@H]1CC3(CC3)NC1=O)C(N)=O)C2(C)C ; C[C@H](OC(C)(C)C)[C@H](NC(=O)C(F)(F)F)C(=O)N1C[C@H]2[C@@H]([C@H]1C(=O)N[C@H](C#N)C[C@@H]1CC3(CC3)NC1=O)C2(C)C]

Step 1: methyl (2S)-2-[[(1R,2S,5S)-6,6-dimethyl-3-azabicyclo[3.1.0]hexane-2-carbonyl]amino]-3-[(6R)-5-oxo-4-azaspiro[2.4]heptan-6-yl]propanoate

A mixture of tert-butyl (1R,2S,5S)-2-[[(1S)-2-methoxy-2-oxo-1-[[(6R)-5-oxo-4-azaspiro[2.4]heptan-6-yl]methyl]ethyl]carbamoyl]-6,6-dimethyl-3-azabicyclo[3.1.0]hexane-3-carboxylate (300 mg, 667.35 umol, 1 eq) was added HCl/MeOH (4 M, 12.00 mL, 71.93 eq). The mixture was stirred at 25° C. for 1 h. Upon completion, the reaction mixture was concentrated under reduced pressure to give methyl (2S)-2-[[(1R,2S,5S)-6,6-dimethyl-3-azabicyclo[3.1.0]hexane-2-carbonyl]amino]-3-[(6R)-5-oxo-4-azaspiro[2.4]heptan-6-yl]propanoate (250 mg, 647.86 umol, 97.08% yield, HCl) was obtained as a colourless oil.

Step 2: methyl (2S)-2-[[(1R,2S,5S)-3-[(2S,3S)-3-tert-butoxy-2-(9H-fluoren-9-ylmethoxycarbonylamino)butanoyl]-6,6-dimethyl-3-azabicyclo[3.1.0]hexane-2-carbonyl]amino]-3-[(6R)-5-oxo-4-azaspiro[2.4]heptan-6-yl]propanoate

To a mixture of (2S,3S)-3-tert-butoxy-2-(9H-fluoren-9-ylmethoxycarbonylamino)butanoic acid (194.67 mg, 489.78 umol, 0.9 eq) in DMF (15 mL) was added DIEA (211.00 mg, 1.63 mmol, 284.37 uL, 3 eq), and then methyl (2S)-2-[[(1R,2S,5S)-6,6-dimethyl-3-azabicyclo[3.1.0]hexane-2-carbonyl]amino]-3-[(6R)-5-oxo-4-azaspiro[2.4]heptan-6-yl]propanoate (210 mg, 544.20 umol, 1 eq, HCl) and HATU (413.85 mg, 1.09 mmol, 2 eq) was added. The mixture was stirred at 25° C. for 1 h. Upon completion, the reaction mixture was diluted with H₂O (20 mL) and extracted with EA 40 mL (20 mL 2). The combined organic layers were washed with brine 20 mL (20 mL*1), dried over Na₂SO₄, filtered and concentrated under reduced pressure to give a residue. The residue was purified by column chromatography (SiO₂, petroleum ether:ethyl acetate=5:1 to 0:1). Compound methyl (2S)-2-[[(1R,2S,5S)-3-[(2S,3S)-3-tert-butoxy-2-(9H-fluoren-9-ylmethoxycarbonylamino)butanoyl]-6,6-dimethyl-3-azabicyclo[3.1.0]hexane-2-carbonyl]amino]-3-[(6R)-5-oxo-4-azaspiro[2.4]heptan-6-yl]propanoate (340 mg, 466.47 umol, 85.72% yield) was obtained as a colourless oil. MS (ESI) m/z 729.4 [M+H]⁺.

Step 3: (1R,2S,5S)-3-[(2S,3S)-2-amino-3-tert-butoxy-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide

A solution of methyl (2S)-2-[[(1R,2S,5S)-3-[(2S,3S)-3-tert-butoxy-2-(9H-fluoren-9-ylmethoxycarbonylamino)butanoyl]-6,6-dimethyl-3-azabicyclo[3.1.0]hexane-2-carbonyl]amino]-3-[(6R)-5-oxo-4-azaspiro[2.4]heptan-6-yl]propanoate (340 mg, 466.47 umol, 1 eq) in NH₃/MeOH (7 M, 60.00 mL, 900.37 eq). The reaction mixture was stirred at 40° C. for 36 h. Upon completion, the reaction mixture was concentrated under reduced pressure to give a residue. The residue was purified by prep-TLC (SiO₂, Petroleum ether:Ethyl acetate=0:1). Compound (1R,2S,5S)-3-[(2S,3S)-2-amino-3-tert-butoxy-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide (171 mg, 347.83 umol, 74.57% yield) was obtained as a colourless oil. MS (ESI) m/z 492.4 [M+H]⁺.

Step 4: (1R,2S,5S)—N-[(1S)-2-amino-2-oxo-1-[[(6R)-5-oxo-4-azaspiro[2.4]heptan-6-yl]methyl]ethyl]-3-[(2S,3S)-3-tert-butoxy-2-[(2,2,2-trifluoroacetyl)amino]butanoyl]-6,6-dimethyl-3-azabicyclo[3.1.0]hexane-2-carboxamide

To a solution of (1R,2S,5S)-3-[(2S,3S)-2-amino-3-tert-butoxy-butanoyl]-N-[(1S)-2-amino-2-oxo-1-[[(6R)-5-oxo-4-azaspiro[2.4]heptan-6-yl]methyl]ethyl]-6,6-dimethyl-3-azabicyclo[3.1.0]hexane-2-carboxamide (160 mg, 325.45 umol, 1 eq) in DCM (8 mL) was added DIEA (126.19 mg, 976.36 umol, 170.06 uL, 3 eq). To the resulting mixture TFAA (136.71 mg, 650.91 umol, 90.54 uL, 2 eq) in DCM (2 mL) was added dropwise at 0° C., and then stirred at 0° C. for 0.5 h. Upon completion, the reaction mixture was diluted with H₂O 20 mL and extracted with DCM 40 mL (20 mL*2). The combined organic layers were washed with brine 20 mL (20 mL*1), dried over Na₂SO₄, filtered and concentrated under reduced pressure to give (1R,2S,5S)—N-[(1S)-2-amino-2-oxo-1-[[(6R)-5-oxo-4-azaspiro[2.4]heptan-6-yl]methyl]ethyl]-3-[(2S,3S)-3-tert-butoxy-2-[(2,2,2-trifluoroacetyl)amino]butanoyl]-6,6-dimethyl-3-azabicyclo[3.1.0]hexane-2-carboxamide (185 mg, 273.90 umol, 84.16% yield, 87% purity) was obtained as a colourless oil.

Step 5: (1R,2S,5S)-3-[(2S,3S)-3-tert-butoxy-2-[(2,2,2-trifluoroacetyl)amino]butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide

To a mixture of (1R,2S,5S)—N-[(1S)-2-amino-2-oxo-1-[[(6R)-5-oxo-4-azaspiro[2.4]heptan-6-yl]methyl]ethyl]-3-[(2S,3S)-3-tert-butoxy-2-[(2,2,2-trifluoroacetyl)amino]butanoyl]-6,6-dimethyl-3-azabicyclo[3.1.0]hexane-2-carboxamide (175 mg, 297.81 umol, 1 eq) in DCM (15 mL) was added burgess reagent (212.91 mg, 893.42 umol, 3 eq). The mixture was stirred at 25° C. for 1 h. Upon completion, the reaction mixture was quenched by addition H₂O (20 mL) at 25° C., and then extracted with DCM (40 mL; 20 mL*2). The combined organic layers were washed with brine 20 mL (20 mL*1), dried over Na₂SO₄, filtered and concentrated under reduced pressure to give a residue. The residue was purified by neutral prep-HPLC(column: Phenomenex C18 75*30 mm*3 um; mobile phase: [water(NH₄HCO₃)-ACN]; B %: 35%-55%, 8 min) to give (1R,2S,5S)-3-[(2S,3S)-3-tert-butoxy-2-[(2,2,2-trifluoroacetyl)amino]butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (70 mg, 122.89 umol, 41.27% yield, 100% purity) was obtained as a yellow solid. MS (ESI) m/z 570.2 [M+H]⁺.

¹H NMR (400 MHz, DMSO-d₆) δ=9.73 (br d, J=5.1 Hz, 1H), 9.13-9.01 (m, 1H), 7.79-7.75 (m, 1H), 4.90-4.82 (m, 1H), 4.37 (br s, 1H), 4.11 (s, 1H), 4.06-3.99 (m, 1H), 3.93 (dd, J=5.5, 10.6 Hz, 1H), 3.72 (d, J=10.7 Hz, 1H), 2.58 (br dd, J=4.6, 10.1 Hz, 1H), 2.25-2.09 (m, 1H), 2.00-1.91 (m, 2H), 1.83 (ddd, J=5.8, 10.2, 13.6 Hz, 1H), 1.64 1.50 (m, 1H), 1.27 (d, J=7.6 Hz, 1H), 1.13-0.99 (m, 15H), 0.87 (s, 3H), 0.77-0.70 (m, 1H), 0.65-0.50 (m, 3H).

Example 83: Synthesis of (1R,2S,5S)—N-[(1S)-1-cyano-2-[(3R)-5,5-dimethyl-2-oxo-pyrrolidin-3-yl]ethyl]-3-[(2S,3R)-3-(3,3-difluorocyclobutoxy)-2-[(2,2,2-trifluoroacetyl)amino]butanoyl]-6,6-dimethyl-3-azabicyclo[3.1.0]hexane-2-carboxamide (Compound 330)

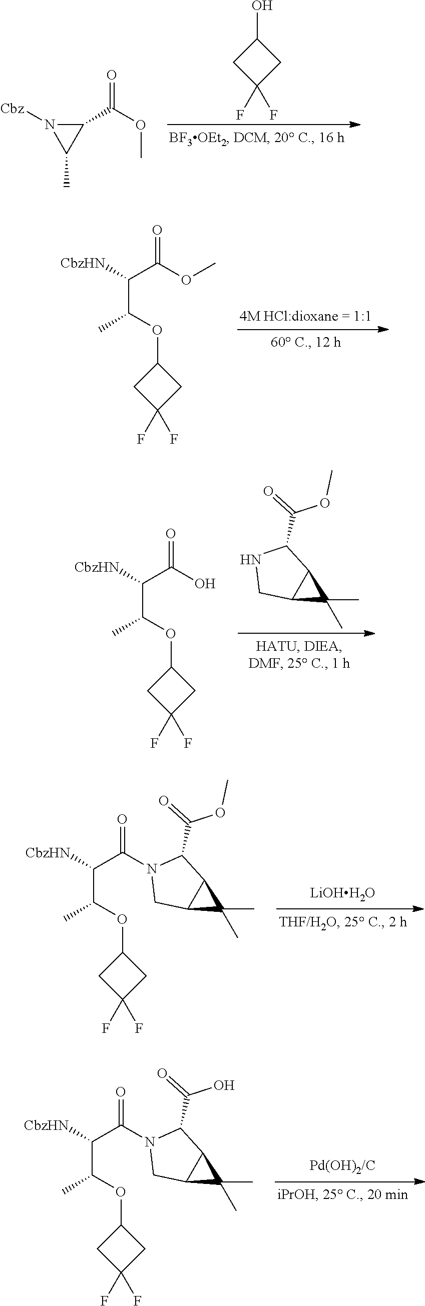

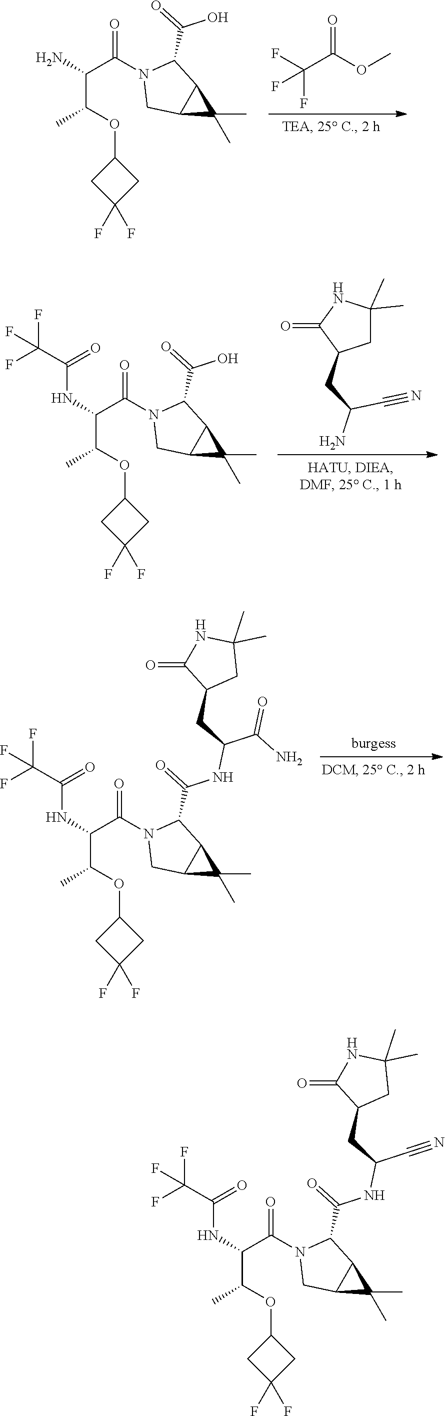

Step 1: methyl (2S,3R)-2-(benzyloxycarbonylamino)-3-(3,3-difluorocyclobutoxy)butanoate

To a solution of O1-benzyl O2-methyl (2S,3S)-3-methylaziridine-1,2-dicarboxylate (3 g, 12.04 mmol, 1 eq) and 3,3-difluorocyclobutanol (1.56 g, 14.44 mmol, 1.2 eq) in CH₃C1(45 mL) was added BF₃.Et₂O (363.44 mg, 1.20 mmol, 316.04 uL, 47% purity, 0.1 eq), and the mixture was stirred at 20° C. for 16 h. Upon completion, the reaction mixture was quenched by addition H₂O (100 mL) at 0° C., and then washed with 10% aqueous citric acid solution (2*100 mL) and brine (2*100 mL), and then extracted with DCM (200 mL), dried over Na₂SO₄, filtered and concentrated under reduced pressure to give a residue. The residue was purified by column chromatography (SiO₂, Petroleum ether:Ethyl acetate=50:1 to 20:1). Compound methyl (2S,3R)-2-(benzyloxycarbonylamino)-3-(3,3-difluorocyclobutoxy)butanoate (2.03 g, 5.68 mmol, 47.20% yield) was obtained as a colourless oil. MS (ESI) m/z 358.2 [M+H]⁺.

Step 2: (2S,3R)-2-(benzyloxycarbonylamino)-3-(3,3-difluorocyclobutoxy)butanoic acid

A solution of methyl (2S,3R)-2-(benzyloxycarbonylamino)-3-(3,3-difluorocyclobutoxy)butanoate (1.9 g, 2.71 mmol, 1 eq) in dioxane (95.00 mL) was added HCl (4 M, 95.00 mL, 71.47 eq), and the mixture was stirred at 60° C. for 12 h. Upon completion, the reaction mixture was concentrated under reduced pressure to give a residue and used directly next step. Compound (2S,3R)-2-(benzyloxycarbonylamino)-3-(3,3-difluorocyclobutoxy)butanoic acid (1.7 g, crude) was obtained as a colourless oil. MS (ESI) m/z 344.2 [M+H]⁺.

Step 3: methyl (1R,2S,5S)-3-[(2S,3R)-2-(benzyloxycarbonylamino)-3-(3,3-difluorocyclobutoxy)butanoyl]-6,6-dimethyl-3-azabicyclo[3.1.0]hexane-2-carboxylate

To a mixture of (2S,3R)-2-(benzyloxycarbonylamino)-3-(3,3-difluorocyclobutoxy)butanoic acid (1.7 g, 4.96 mmol, 1 eq) in DMF (74 mL) was added DIEA (1.92 g, 14.85 mmol, 2.58 mL, 3 eq), and then methyl (1R,2S,5S)-6,6-dimethyl-3-azabicyclo[3.1.0]hexane-2-carboxylate (837.92 mg, 4.96 mmol, 1 eq) and HATU (3.766 g, 9.90 mmol, 2 eq) was added. The mixture was stirred at 25° C. for 1 h. Upon completion, the reaction mixture was diluted with H₂O (100 mL) and extracted with EA (100 mL*2). The combined organic layers were washed with brine (100 mL), dried over Na₂SO₄, filtered and concentrated under reduced pressure to give a residue. The residue was purified by column chromatography (SiO₂, Petroleum ether:Ethyl acetate=10:1 to Ethyl acetate:MeOH=15:1). Compound methyl (1R,2S,5S)-3-[(2S,3R)-2-(benzyloxycarbonylamino)-3-(3,3-difluorocyclobutoxy)butanoyl]-6,6-dimethyl-3-azabicyclo[3.1.0]hexane-2-carboxylate (2.0 g, 3.09 mmol, 81.71% yield, 76.5% purity) was obtained as a colourless oil. MS (ESI) m/z 495.3 [M+H]⁺.

Step 4: (1R,2S,5S)-3-[(2S,3R)-2-(benzyloxycarbonylamino)-3-(3,3-difluorocyclobutoxy)butanoyl]-6,6-dimethyl-3-azabicyclo[3.1.0]hexane-2-carboxylic acid

A solution of methyl (1R,2S,5S)-3-[(2S,3R)-2-(benzyloxycarbonylamino)-3-(3,3-difluorocyclobutoxy)butanoyl]-6,6-dimethyl-3-azabicyclo[3.1.0]hexane-2-carboxylate (1.8 g, 3.64 mmol, 1 eq) in THE (54 mL) and H₂O (18 mL) was added LiOH.H₂O (305.48 mg, 7.28 mmol, 2 eq), the mixture was stirred at 25° C. for 2 h. Upon completion, the reaction mixture was adjusted pH=3 by HCl (10%), and then diluted with H₂O (50 mL) and extracted with DCM (50 mL*2). The combined organic layers were washed with brine (50 mL), dried over Na₂SO₄, filtered and concentrated under reduced pressure to give a residue. The residue was used next step directly. Compound (1R,2S,5S)-3-[(2S,3R)-2-(benzyloxycarbonylamino)-3-(3,3-difluorocyclobutoxy)butanoyl]-6,6-dimethyl-3-azabicyclo[3.1.0]hexane-2-carboxylic acid (1.6 g, 3.33 mmol, 91.48% yield) was obtained as a colourless oil. MS (ESI) m/z 481.3 [M+H]⁺.

Step 5: (1R,2S,5S)-3-[(2S,3R)-2-amino-3-(3,3-difluorocyclobutoxy)butanoyl]-6,6-dimethyl-3-azabicyclo[3.1.0]hexane-2-carboxylic acid

To a solution of (1R,2S,5S)-3-[(2S,3R)-2-(benzyloxycarbonylamino)-3-(3,3-difluorocyclobutoxy)butanoyl]-6,6-dimethyl-3-azabicyclo[3.1.0]hexane-2-carboxylic acid (1.6 g, 3.33 mmol, 1 eq) in IPA (8 mL) was added Pd(OH)₂/C (2.34 g, 3.33 mmol, 20% purity, 1 eq) under H₂ (15 Psi). The mixture was stirred at 25° C. for 20 min. Upon completion, the reaction mixture was filtered to get the filtrate, then the filtrate was concentrated to get the residue. The residue was used next step directly. Compound (1R,2S,5S)-3-[(2S,3R)-2-amino-3-(3,3-difluorocyclobutoxy)butanoyl]-6,6-dimethyl-3-azabicyclo[3.1.0]hexane-2-carboxylic acid (860 mg, 2.48 mmol, 74.56% yield) was obtained as a colourless oil. MS (ESI) m/z 347.3 [M+H]⁺.

Step 6: (1R,2S,5S)-3-[(2S,3R)-3-(3,3-difluorocyclobutoxy)-2-[(2,2,2-trifluoroacetyl)amino]butanoyl]-6,6-dimethyl-3-azabicyclo[3.1.0]hexane-2-carboxylic acid

To a mixture of (1R,2S,5S)-3-[(2S,3R)-2-amino-3-(3,3-difluorocyclobutoxy)butanoyl]-6,6-dimethyl-3-azabicyclo[3.1.0]hexane-2-carboxylic acid (860 mg, 2.48 mmol, 1 eq) and methyl 2,2,2-trifluoroacetate (12.70 g, 99.18 mmol, 10 mL, 39.95 eq) was added TEA (753.73 mg, 7.45 mmol, 1.04 mL, 3 eq). After the addition, the resulting mixture was stirred at 25° C. for 2 h. Upon completion, the residue was purified by prep-HPLC (column: Phenomenex Luna 80*30 mm*3 um; mobile phase: [water (HCl)-ACN]; B %: 35%-60%, 8 min). Compound (1R,2S,5S)-3-[(2S,3R)-3-(3,3-difluorocyclobutoxy)-2-[(2,2,2-trifluoroacetyl)amino]butanoyl]-6,6-dimethyl-3-azabicyclo[3.1.0]hexane-2-carboxylic acid (710 mg, 79.12 umol, 87.50% yield, 100% purity) was obtained as a white solid. MS (ESI) m/z 443.1 [M+H]⁺.

¹H NMR (400 MHz, DMSO-d₆) δ=12.81 (br s, 1H), 9.94 (d, J=8.0 Hz, 1H), 4.45 (t, J=8.4 Hz, 1H), 4.15-4.06 (m, 2H), 3.93-3.71 (m, 3H), 2.96-2.70 (m, 2H), 2.56 (td, J=2.5, 5.5 Hz, 1H), 2.44-2.34 (m, 1H), 1.54 (dd, J=5.0, 7.5 Hz, 1H), 1.45 (d, J=7.5 Hz, 1H), 1.18 (d, J=6.2 Hz, 3H), 1.06-0.99 (m, 3H), 0.83 (s, 3H).

Step 7: (1R,2S,5S)—N-[(1S)-2-amino-1-[[(3R)-5,5-dimethyl-2-oxo-pyrrolidin-3-yl]methyl]-2-oxo-ethyl]-3-[(2S,3R)-3-(3,3-difluorocyclobutoxy)-2-[(2,2,2-trifluoroacetyl)amino]butanoyl]-6,6-dimethyl-3-azabicyclo[3.1.0]hexane-2-carboxamide

To a solution of (1R,2S,5S)-3-[(2S,3R)-3-(3,3-difluorocyclobutoxy)-2-[(2,2,2-trifluoroacetyl)amino]butanoyl]-6,6-dimethyl-3-azabicyclo[3.1.0]hexane-2-carboxylic acid (700 mg, 1.58 mmol, 1 eq) and (2S)-2-amino-3-[(3R)-5,5-dimethyl-2-oxo-pyrrolidin-3-yl]propanamide (314.81 mg, 1.58 mmol, 1 eq) in DMF (30 mL) was added HATU (1.20 g, 3.16 mmol, 2 eq) and DIEA (612.60 mg, 4.74 mmol, 825.60 uL, 3 eq). The mixture was stirred at 25° C. for 1 h. Upon completion, the residue was diluted with H₂O (100 mL) and extracted with EA (100 mL). The combined organic layers were washed with brine (100 mL), dried over Na₂SO₄, filtered and concentrated under reduced pressure to give a residue. The residue was purified by column chromatography (SiO₂, Petroleum ether:Ethyl acetate=10:1 to Ethyl acetate:MeOH=15:1). Compound (1R,2S,5S)—N-[(1S)-2-amino-1-[[(3R)-5,5-dimethyl-2-oxo-pyrrolidin-3-yl]methyl]-2-oxo-ethyl]-3-[(2S,3R)-3-(3,3-difluorocyclobutoxy)-2-[(2,2,2-trifluoroacetyl)amino]butanoyl]-6,6-dimethyl-3-azabicyclo[3.1.0]hexane-2-carboxamide (530 mg, 616.17 umol, 39.00% yield, 72.5% purity) was obtained as a colourless oil. MS (ESI) m/z 624.5 [M+H]⁺.

Step 8: (1R,2S,5S)—N-[(1S)-1-cyano-2-[(3R)-5,5-dimethyl-2-oxo-pyrrolidin-3-yl]ethyl]-3-[(2S,3R)-3-(3,3-difluorocyclobutoxy)-2-[(2,2,2-trifluoroacetyl)amino]butanoyl]-6,6-dimethyl-3-azabicyclo[3.1.0]hexane-2-carboxamide

To a solution of (1R,2S,5S)—N-[(1S)-2-amino-1-[[(3R)-5,5-dimethyl-2-oxo-pyrrolidin-3-yl]methyl]-2-oxo-ethyl]-3-[(2S,3R)-3-(3,3-difluorocyclobutoxy)-2-[(2,2,2-trifluoroacetyl)amino]butanoyl]-6,6-dimethyl-3-azabicyclo[3.1.0]hexane-2-carboxamide (530 mg, 849.89 umol, 1 eq) in DCM (10 mL) was added burgess reagent (607.60 mg, 2.55 mmol, 3 eq). The mixture was stirred at 25° C. for 2 h. Upon completion, the residue was quenched by addition H₂O (20 mL) at 25° C., and then extracted with DCM (20 mL*2). The combined organic layers were washed with brine (40 mL), dried over Na₂SO₄, filtered and concentrated under reduced pressure to give a residue. The residue was purified by prep-HPLC (column: Phenomenex C18 75*30 mm*3 um; mobile phase: [water (NH₄HCO₃)-ACN]; B %: 35% 55%, 8 min). Compound (1R,2S,5S)—N-[(1S)-1-cyano-2-[(3R)-5,5-dimethyl-2-oxo-pyrrolidin-3-yl]ethyl]-3-[(2S,3R)-3-(3,3-difluorocyclobutoxy)-2-[(2,2,2-trifluoroacetyl)amino]butanoyl]-6,6-dimethyl-3-azabicyclo[3.1.0]hexane-2-carboxamide (300 mg, 495.38 umol, 58.29% yield, 100% purity) was obtained as a white solid. MS (ESI) m/z 606.1 [M+H]⁺.

¹H NMR (400 MHz, DMSO-d₆) δ=9.93 (br s, 1H), 9.01 (d, J=8.5 Hz, 1H), 7.81 (s, 1H), 4.92 (ddd, J=5.5, 8.6, 10.4 Hz, 1H), 4.42 (br d, J=8.9 Hz, 1H), 4.19-4.02 (m, 2H), 3.94 (dd, J=5.4, 10.4 Hz, 1H), 3.82-3.66 (m, 2H), 2.94-2.66 (m, 2H), 2.63-2.53 (m, 2H), 2.43-2.32 (m, 1H), 2.14 (ddd, J=4.5, 10.5, 13.6 Hz, 1H), 1.97 (dd, J=8.5, 12.2 Hz, 1H), 1.72 (ddd, J=5.4, 10.4, 13.6 Hz, 1H), 1.61-1.47 (m, 2H), 1.33 (d, J=7.5 Hz, 1H), 1.22-1.10 (m, 9H), 1.03 (s, 3H), 0.85 (s, 3H).

Example 84: Synthesis of (1R,2S,5S)—N-[(1S)-1-cyan-2-[(6R)-5-oxo-4-azaspiro[2.4]heptan-6-yl]ethyl]-3-[(2S,3R)-3-(1-cyclopropykyclopropoxy)-2-[(2,2,2-trifluoroacetyl)amino]butanoyl]-6,6-dimethyl-3-azabicyclo[3.1.0]hexane-2-carboxamide (Compound 331)

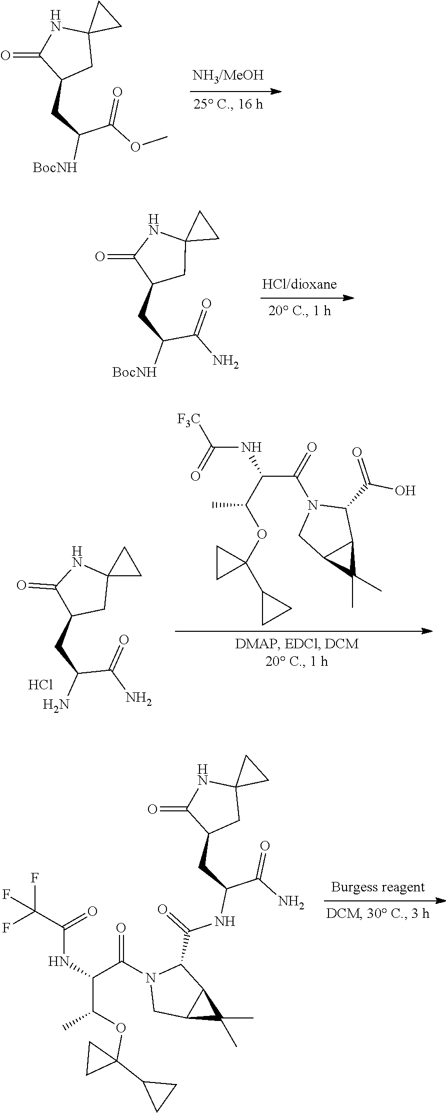 [SMILES: COC(=O)[C@H](C[C@@H]1CC2(CC2)NC1=O)NC(=O)OC(C)(C)C ; CC(C)(C)OC(=O)N[C@@H](C[C@@H]1CC2(CC2)NC1=O)C(N)=O ; NC(=O)[C@@H](N)C[C@@H]1CC2(CC2)NC1=O ; Cl ; C[C@@H](OC1(C2CC2)CC1)[C@H](NC(=O)C(F)(F)F)C(=O)N1C[C@H]2[C@@H]([C@H]1C(=O)O)C2(C)C ; C[C@@H](OC1(C2CC2)CC1)[C@H](NC(=O)C(F)(F)F)C(=O)N1C[C@H]2[C@@H]([C@H]1C(=O)N[C@@H](C[C@@H]1CC3(CC3)NC1=O)C(N)=O)C2(C)C]

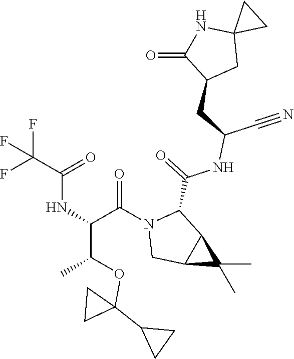 [SMILES: C[C@@H](OC1(C2CC2)CC1)[C@H](NC(=O)C(F)(F)F)C(=O)N1C[C@H]2[C@@H]([C@H]1C(=O)N[C@H](C#N)C[C@@H]1CC3(CC3)NC1=O)C2(C)C]

Step 1: tert-butyl N-[(1S)-2-amino-2-oxo-1-[[(6R)-5-oxo-4-azaspiro[2.4]heptan-6-yl]methyl]ethyl]carbamate

A solution of methyl (2S)-2-(tert-butoxycarbonylamino)-3-[(6R)-5-oxo-4-azaspiro[2.4]heptan-6-yl]propanoate (2 g, 5.76 mmol, 90% purity, 1 eq) in NH₃/MeOH (7 M, 40 mL, 48.59 eq) was stirred at 25° C. for 16 h. Upon completion, the reaction mixture was concentrated under reduced pressure to give tert-butyl N-[(1S)-2-amino-2-oxo-1-[[(6R)-5-oxo-4-azaspiro[2.4]heptan-6-yl]methyl]ethyl]carbamate (1.7 g, crude) as a white solid. MS (ESI) m/z 298.3 [M+H]⁺.

Step 2: (2S)-2-amino-3-[(6R)-5-oxo-4-azaspiro[2.4]heptan-6-yl]propanamide

A solution of tert-butyl N-[(1S)-2-amino-2-oxo-1-[[(6R)-5-oxo-4-azaspiro[2.4]heptan-6-yl]methyl]ethyl]carbamate (1.3 g, 3.50 mmol, 80% purity, 1 eq) in HCl/dioxane (4 M, 10.40 mL, 11.89 eq) was stirred at 20° C. for 1 h. Upon completion, the mixture was concentrated under reduced pressure to give (2S)-2-amino-3-[(6R)-5-oxo-4-azaspiro[2.4]heptan-6-yl]propanamide (800 mg, crude, HCl) as a white solid. MS (ESI) m/z 198.3 [M+H]⁺.

Step 3: (1R,2S,5S)—N-[(1S)-2-amino-2-oxo-1-[[(6R)-5-oxo-4-azaspiro[2.4]heptan-6-yl]methyl]ethyl]-3-[(2S,3R)-3-(1-cyclopropylcyclopropoxy)-2-[(2,2,2-trifluoroacetyl)amino]butanoyl]-6,6-dimethyl-3-azabicyclo[3.1.0]hexane-2-carboxamide

To a solution of (1R,2S,5S)-3-[(2S,3R)-3-(1-cyclopropylcyclopropoxy)-2-[(2,2,2-trifluoroacetyl)amino]butanoyl]-6,6-dimethyl-3-azabicyclo[3.1.0]hexane-2-carboxylic acid (986.89 mg, 2.28 mmol, 1 eq) in DCM (10 mL) was added (2S)-2-amino-3-[(6R)-5-oxo-4-azaspiro[2.4]heptan-6-yl]propanamide (800 mg, 2.74 mmol, 80% purity, 1.2 eq, HCl) and DMAP (836.44 mg, 6.85 mmol, 3 eq), followed by EDCI (874.99 mg, 4.56 mmol, 2 eq). The mixture was stirred at 20° C. for 1 h. Upon completion, the reaction mixture was quenched with water (10 mL) and extracted with DCM (10 mL*3). The combined organic layer was washed by 1 N HCl (50 mL), then washed by NaHCO₃(50 mL), dried over Na₂SO₄, filtered and concentrated under reduced pressure to give a residue. The residue was purified by column chromatography (SiO₂, DCM:MeOH=100:1 to 10:1) to give (1R,2S,5S)—N-[(1S)-2-amino-2-oxo-1-[[(6R)-5-oxo-4-azaspiro[2.4]heptan-6-yl]methyl]ethyl]-3-[(2S,3R)-3-(1-cyclopropylcyclopropoxy)-2-[(2,2,2-trifluoroacetyl)amino]butanoyl]-6,6-dimethyl-3-azabicyclo[3.1.0]hexane-2-carboxamide (900 mg, 1.18 mmol, 51.58% yield, 80% purity) as a white solid.

Step 4: (1R,2S,5S)—N-[(1S)-1-cyano-2-[(6R)-5-oxo-4-azaspiro[2.4]heptan-6-yl]ethyl]-3-[(2S,3R)-3-(1-cyclopropylcyclopropoxy)-2-[(2,2,2-trifluoroacetyl)amino]butanoyl]-6,6-dimethyl-3-azabicyclo[3.1.0]hexane-2-carboxamide

A solution of (1R,2S,5S)—N-[(1S)-2-amino-2-oxo-1-[[(6R)-5-oxo-4-azaspiro[2.4]heptan-6-yl]methyl]ethyl]-3-[(2S,3R)-3-(1-cyclopropylcyclopropoxy)-2-[(2,2,2-trifluoroacetyl)amino]butanoyl]-6,6-dimethyl-3-azabicyclo[3.1.0]hexane-2-carboxamide (900 mg, 1.18 mmol, 80% purity, 1 eq) in DCM (9 mL) was added burgess reagent (841.57 mg, 3.53 mmol, 3 eq). The mixture was stirred at 30° C. for 3 h. The mixture was concentrated under reduced pressure to give a residue. The residue was purified by prep-HPLC (column: Waters Xbridge BEH C18 250*50 mm*10 um; mobile phase: [water(NH₄HCO₃)-ACN]; B %: 40%-60%, 10 min) to give (1R,2S,5S)—N-[(1S)-1-cyano-2-[(6R)-5-oxo-4-azaspiro[2.4]heptan-6-yl]ethyl]-3-[(2S,3R)-3-(1-cyclopropylcyclopropoxy)-2-[(2,2,2-trifluoroacetyl)amino]butanoyl]-6,6-dimethyl-3-azabicyclo[3.1.0]hexane-2-carboxamide (190 mg, 316.86 umol, 26.92% yield, 99% purity) as a white solid. MS (ESI) m/z 592.1 [M−H]⁺.

¹H NMR (400 MHz, DMSO-d₆) δ=9.82 (br s, 1H), 9.00 (d, J=8.5 Hz, 1H), 7.76 (s, 1H), 4.95 (ddd, J=5.6, 8.5, 10.4 Hz, 1H), 4.38 (d, J=8.6 Hz, 1H), 4.13 (s, 1H), 4.06-3.97 (m, 1H), 3.93 (dd, J=5.4, 10.4 Hz, 1H), 3.67 (d, J=10.5 Hz, 1H), 2.71-2.59 (m, 1H), 2.18 (ddd, J=4.7, 10.4, 13.5 Hz, 1H), 2.04-1.88 (m, 2H), 1.83 (ddd, J=5.5, 10.5, 13.5 Hz, 1H), 1.56 (dd, J=5.5, 7.4 Hz, 1H), 1.44-1.36 (m, 1H), 1.33 (d, J=7.6 Hz, 1H), 1.24 (d, J=6.1 Hz, 3H), 1.02 (s, 3H), 0.83 (s, 3H), 0.78-0.72 (m, 1H), 0.67 (td, J=5.6, 11.1 Hz, 1H), 0.62-0.54 (m, 3H), 0.52-0.41 (m, 3H), 0.36-0.29 (m, 1H), 0.23-0.16 (m, 1H), 0.12 0.06 (m, 1H), 0.04--0.04 (m, 1H)

Example 85: Synthesis of (1R,2S,5S)—N-[(1S)-1-cyano-2-[(7R)-6-oxo-5-azaspiro[3.4]octan-7-yl]ethyl]-3-[(2S,3R)-3-methoxy-2-[(2,2,2-trifluoroacetyl)amino]butanoyl]-6,6-dimethyl-3-azabicyclo[3.1.0]hexane-2-carboxamide (Compound 425)

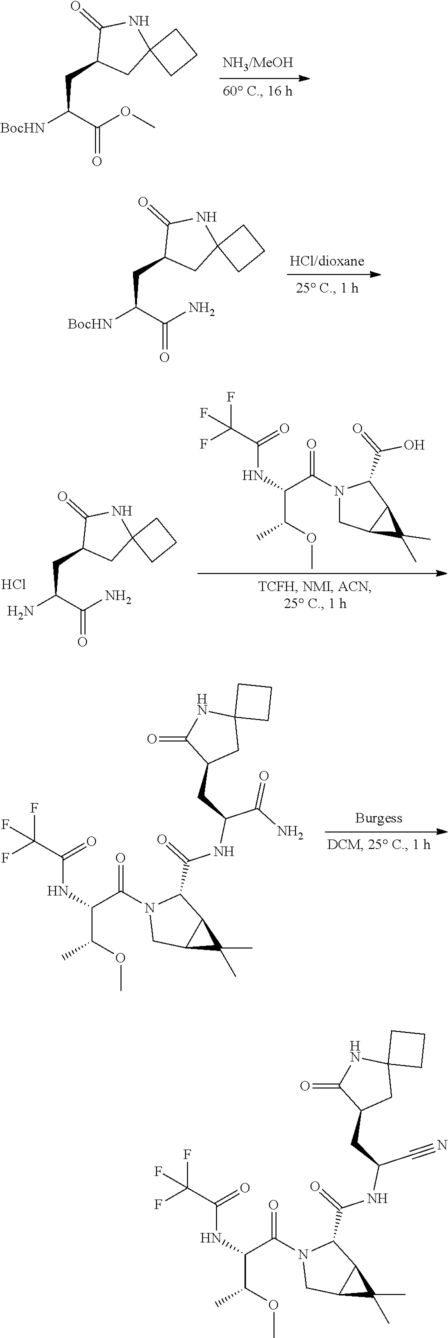

Step 1: tert-butyl N-[(1S)-2-amino-2-oxo-1-[[(7R)-6-oxo-5-azaspiro[3.4]octan-7-yl]methyl]ethyl]carbamate

A solution of methyl (2S)-2-(tert-butoxycarbonylamino)-3-[(7R)-6-oxo-5-azaspiro[3.4]octan-7-yl]propanoate (1 g, 3.06 mmol, 1 eq) in NH₃/MeOH (7 M, 50 mL) was stirred at 60° C. for 16 h. Upon completion, the reaction mixture was concentrated under reduced pressure to give the product tert-butyl N-[(1S)-2-amino-2-oxo-1-[[(7R)-6-oxo-5-azaspiro[3.4]octan-7-yl]methyl]ethyl]carbamate (800 mg, crude) as a white solid. MS (ESI) m/z 312.3 [M+H]⁺

Step 2: (2S)-2-amino-3-[(7R)-6-oxo-5-azaspiro[3.4]octan-7-yl]propanamide

A solution of tert-butyl N-[(1S)-2-amino-2-oxo-1-[[(7R)-6-oxo-5-azaspiro[3.4]octan-7-yl]methyl]ethyl]carbamate (800 mg, 2.57 mmol, 1 eq) in HCl/dioxane (4 M, 10 mL) was stirred at 25° C. for 1 h. Upon completion, the reaction mixture was concentrated under reduced pressure to give the product (2S)-2-amino-3-[(7R)-6-oxo-5-azaspiro[3.4]octan-7-yl]propanamide (700 mg, crude, HCl) as a light yellow solid. MS (ESI) m/z 212.3 [M+H]⁺

Step 3: (1R,2S,5S)—N-[(1S)-2-amino-2-oxo-1-[[(7R)-6-oxo-5-azaspiro[3.4]octan-7-yl]methyl]ethyl]-3-[(2S,3R)-3-methoxy-2-[(2,2,2-trifluoroacetyl)amino]butanoyl]-6,6-dimethyl-3-azabicyclo[3.1.0]hexane-2-carboxamide

To a solution of (2S)-2-amino-3-[(7R)-6-oxo-5-azaspiro[3.4]octan-7-yl]propanamide (600 mg, 2.42 mmol, 1 eq, HCl) in ACN (20 mL) was added (1R,2S,5S)-3-[(2S,3R)-3-methoxy-2-[(2,2,2-trifluoroacetyl)amino]butanoyl]-6,6-dimethyl-3-azabicyclo[3.1.0]hexane-2-carboxylic acid (976.01 mg, 2.66 mmol, 1.1 eq), NMI (596.56 mg, 7.27 mmol, 579.18 uL, 3 eq) and then was added TCFH (1.36 g, 4.84 mmol, 2 eq), the mixture was stirred at 25° C. for 1 h. Upon completion, the reaction mixture was diluted with H₂O (60 mL) and then was extracted with EA (20 mL*3). The combined organic layers were dried over Na₂SO₄, filtered and concentrated in vacuum and was purified by column chromatography (SiO₂, Petroleum ether:Ethyl acetate=2:1 to 0:1) to obtained (1R,2S,5S)—N-[(1S)-2-amino-2-oxo-1-[[(7R)-6-oxo-5-azaspiro[3.4]octan-7-yl]methyl]ethyl]-3-[(2S,3R)-3-methoxy-2-[(2,2,2-trifluoroacetyl)amino]butanoyl]-6,6-dimethyl-3-azabicyclo[3.1.0]hexane-2-carboxamide (730 mg, 913.19 umol, 37.70% yield, 70% purity) as a light yellow oil. MS (ESI) m/z 560.4 [M+H]⁺.

Step 4: (1R,2S,5S)—N-[(1S)-1-cyano-2-[(7R)-6-oxo-5-azaspiro[3.4]octan-7-yl]ethyl]-3-[(2S,3R)-3-methoxy-2-[(2,2,2-trifluoroacetyl)amino]butanoyl]-6,6-dimethyl-3-azabicyclo[3.1.0]hexane-2-carboxamide

To a solution of (1R,2S,5S)—N-[(1S)-2-amino-2-oxo-1-[[(7R)-6-oxo-5-azaspiro[3.4]octan-7-yl]methyl]ethyl]-3-[(2S,3R)-3-methoxy-2-[(2,2,2-trifluoroacetyl)amino]butanoyl]-6,6-dimethyl-3-azabicyclo[3.1.0]hexane-2-carboxamide (730 mg, 913.19 umol, 70% purity, 1 eq) in DCM (20 mL) was added burgess reagent (1.31 g, 5.48 mmol, 6 eq), and the mixture was stirred at 25° C. for 1 h. Upon completion, the reaction mixture was quenched by water (1 mL) and was dried by blowing N₂ and was purified by prep-HPLC (column: Waters Xbridge Prep OBD C18 150*40 mm*10 um; mobile phase: [water (NH₄HCO₃)-ACN]; B %: 35%-65%, 8 min) to obtained (1R,2S,5S)—N-[(1S)-1-cyano-2-[(7R)-6-oxo-5-azaspiro[3.4]octan-7-yl]ethyl]-3-[(2S,3R)-3-methoxy-2-[(2,2,2-trifluoroacetyl)amino]butanoyl]-6,6-dimethyl-3-azabicyclo[3.1.0]hexane-2-carboxamide (195.37 mg, 358.91 umol, 39.30% yield, 99.49% purity) as a white solid. MS (ESI) m/z 542.2 [M+H]⁺

¹H NMR (400 MHz, DMSO-d₆) δ=9.86 (br d, J=6.8 Hz, 1H), 9.00 (d, J=8.6 Hz, 1H), 8.15 (s, 1H), 4.96 (ddd, J=5.6, 8.6, 10.6 Hz, 1H), 4.39-4.29 (m, 1H), 4.13 (s, 1H), 3.97-3.88 (m, 1H), 3.87-3.81 (m, 1H), 3.67-3.55 (m, 1H), 3.26-3.19 (m, 3H), 2.47-2.39 (m, 1H), 2.35-2.21 (m, 2H), 2.12 (ddd, J=4.8, 10.6, 13.6 Hz, 1H), 2.06-1.98 (m, 2H), 1.92 (tdd, J=3.8, 7.6, 11.0 Hz, 1H), 1.78-1.66 (m, 2H), 1.66-1.53 (m, 3H), 1.32 (d, J=7.6 Hz, 1H), 1.15 (d, J=6.4 Hz, 3H), 1.03 (s, 3H), 0.88 (s, 3H).

Example 86: Synthesis of (1R,2S,5S)—N-[(1S)-1-cyano-2-[(3R)-5,5-dimethyl-2-oxo-pyrrolidin-3-yl]ethyl]-6,6-dimethyl-3-[(2S,3R)-2-[(2,2,2-trifluoroacetyl)amino]-3-(2,2,2-trifluoro-1,1-dimethyl-ethoxy)butanoyl]-3-azabicyclo[3.1.0]hexane-2-carboxamide (Compound 357)

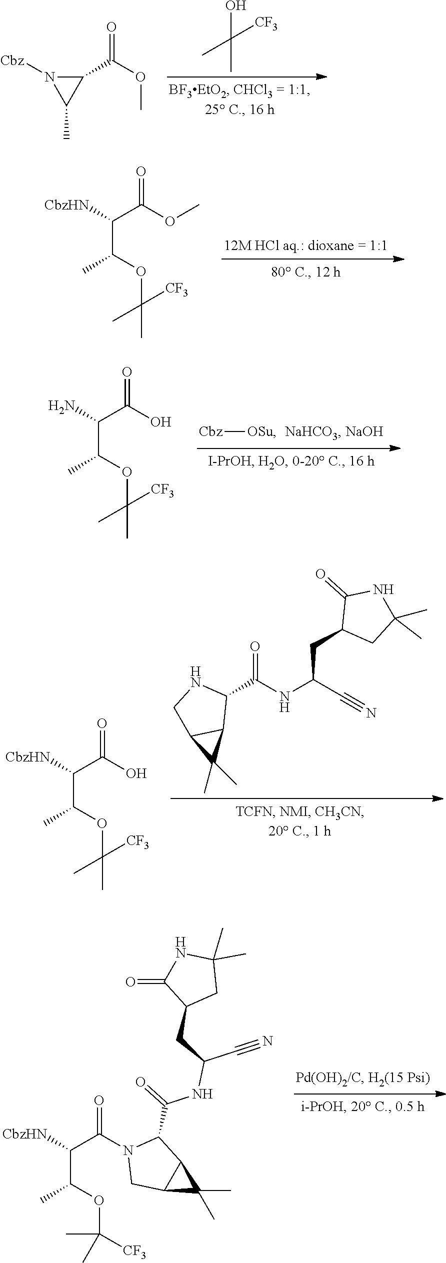

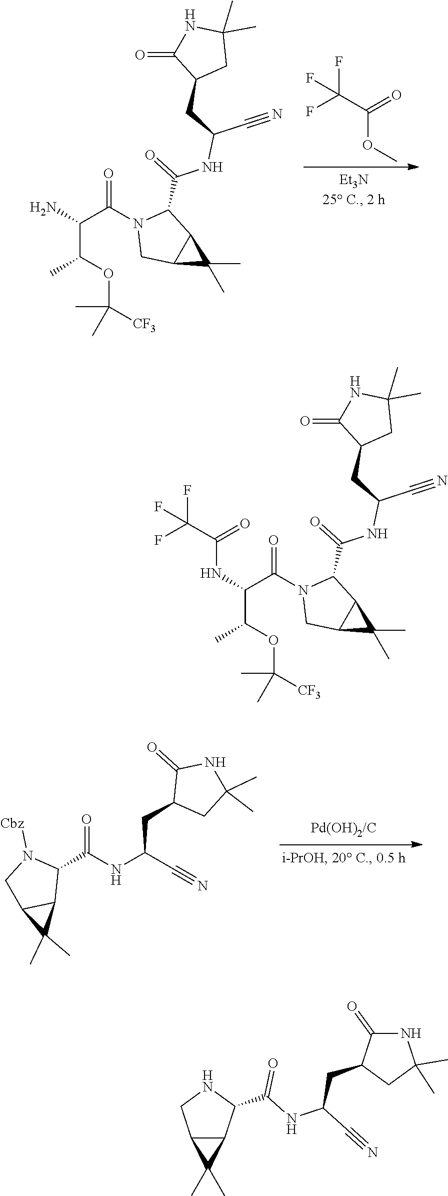

Step 1: (1R,2S,5S)—N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide

To a solution of benzyl (1R,2S,5S)-2-[[(1S)-1-cyano-2-[(3R)-5,5-dimethyl-2-oxo-pyrrolidin-3-yl]ethyl]carbamoyl]-6,6-dimethyl-3-azabicyclo[3.1.0]hexane-3-carboxylate (2 g, 4.42 mmol, 1 eq) in IPA (5 mL) was added Pd(OH)₂ (500 mg, 712.05 umol, 20% purity, 1.61e-1 eq) under N₂. The suspension was degassed under vacuum and purged with H₂ several times. The mixture was stirred under H₂ (15 psi) at 20° C. for 0.5 h. Upon completion, the reaction mixture was filtered and concentrated under reduced pressure to give (1R,2S,5S)—N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (1.3 g, crude) as a white solid, which was used for next step without further purification. MS (ESI) m/z 319.2 [M+H]⁺.

Step 2: methyl (2S,3R)-2-(benzyloxycarbonylamino)-3-(2,2,2-trifluoro-1,1-dimethyl-ethoxy)butanoate

To a solution of 1,1,1-trifluoro-2-methyl-propan-2-ol (8.22 g, 64.19 mmol, 7.03 mL, 1 eq) in CHCl₃ (300 mL) was added O1-benzyl O2-methyl (2S,3S)-3-methylaziridine-1,2-dicarboxylate (4 g, 16.05 mmol, 0.25 eq) and diethyloxonio(trifluoro)boranuide (1.94 g, 6.42 mmol, 1.69 mL, 47% purity, 0.1 eq). The mixture was stirred at 25° C. for 16 h. Upon completion, the reaction was poured into H₂O (150 mL) and then extracted with DCM (100 mL*2). The combined organic phase was washed with brine (100 mL), dried over anhydrous Na₂SO₄, filtered and concentrated in vacuum. The residue was purified by prep-HPLC (column: Xtimate C18 10 u 250 mm*80 mm; mobile phase: [water(NH₄HCO₃)-ACN]; B %: 40%-70%, 25 min) to give methyl (2S,3R)-2-(benzyloxycarbonylamino)-3-(2,2,2-trifluoro-1,1-dimethyl-ethoxy)butanoate (1.6 g, 4.22 mmol, 6.57% yield, 99.5% purity) was as a colorless oil. MS (ESI) m/z 378.2 [M+H]⁺.

Step 3: (2S,3R)-2-amino-3-(2,2,2-trifluoro-1,1-dimethyl-ethoxy)butanoic acid

To a solution of methyl (2S,3R)-2-(benzyloxycarbonylamino)-3-(2,2,2-trifluoro-1,1-dimethyl-ethoxy)butanoate (2.1 g, 5.06 mmol, 91% purity, 1 eq) in dioxane (16 mL) was added HCl (12 M, 19.11 mL, 45.28 eq). The mixture was stirred at 80° C. for 12 h. Upon completion, the residue was concentrated in vacuum to give (2S,3R)-2-amino-3-(2,2,2-trifluoro-1,1-dimethyl-ethoxy)butanoic acid (800 mg, crude, HCl) as a colorless oil. MS (ESI) m/z 230.2[M+H]⁺.

Step 4: (2S,3R)-2-(benzyloxycarbonylamino)-3-(2,2,2-trifluoro-1,1-dimethyl-ethoxy)butanoic acid

10 mL of a buffer-pH=11 (Saturated NaHCO₃(1.30 g, 15.50 mmol, 603.01 uL, 5.15 eq), adjusted with 4 M NaOH (4 M, 602.99 uL, 8.01e-1 eq) to pH=11) was added to a solution of (2S,3R)-2-amino-3-(2,2,2-trifluoro-1,1-dimethyl-ethoxy)butanoic acid (800 mg, 3.01 mmol, 1 eq, HCl) in IPA (8 mL). The reaction mixture was cooled to 0° C. and benzyl(2,5-dioxopyrrolidin-1-yl) carbonate (825.55 mg, 3.31 mmol, 1.1 eq) was added. The reaction mixture was stirred at 20° C. for 16 h. Upon completion, the reaction mixture was adjusted pH to 6 with 1 M HCl. Then the mixture was extracted with ethyl acetate (10 mL 2). The organic phase was washed with brine (5 mL), dried over anhydrous magnesium sulfate, filtered, concentrated under reduced pressure to give reside. The residue was purified by prep-HPLC (column: Phenomenex luna C18 (250*70 mm, 15 um); mobile phase: [water(HCl)-ACN]; B %: 35%-65%, 20 min) to give (2S,3R)-2-(benzyloxycarbonylamino)-3-(2,2,2-trifluoro-1,1-dimethyl-ethoxy)butanoic acid (800 mg, 1.83 mmol, 60.69% yield, 83% purity) as a colorless oil. MS (ESI) m/z 364.2 [M+H]⁺

Step 5: benzyl N-[(1S,2R)-1-[(1R,2S,5S)-2-[[(1S)-1-cyano-2-[(3R)-5,5-dimethyl-2-oxo-pyrrolidin-3-yl]ethyl]carbamoyl]-6,6-dimethyl-3-azabicyclo[3.1.0]hexane-3-carbonyl]-2-(2,2,2-trifluoro-1,1-dimethylethoxy)propyl]carbamate

To a solution of (2S,3R)-2-(benzyloxycarbonylamino)-3-(2,2,2-trifluoro-1,1-dimethyl-ethoxy)butanoic acid (400 mg, 913.77 umol, 83% purity, 1 eq) and (1R,2S,5S)—N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (290.96 mg, 913.77 umol, 1 eq) in ACN (5 mL) was added 1-methylimidazole (225.06 mg, 2.74 mmol, 218.51 uL, 3 eq) and 1-[chloro(pyrrolidin-1-ium-1-ylidene)methyl]pyrrolidine; hexafluorophosphate (607.94 mg, 1.83 mmol, 2 eq). The mixture was stirred at 20° C. for 1 h. Upon completion, the reaction mixture was diluted with water (5 mL) and extected with ethyl acetate (5 mL*2). The organic phase was washed with brine (5 mL), dried over anhydrous magnesium sulfate, filtered, concentrated under reduced pressure. The residue was purified by column chromatography (SiO₂, Petroleum ether/Ethyl acetate=10/1 to 0/1) to give benzyl N-[(1S,2R)-1-[(1R,2S,5S)-2-[[(1S)-1-cyano-2-[(3R)-5,5-dimethyl-2-oxo-pyrrolidin-3-yl]ethyl]carbamoyl]-6,6-dimethyl-3-azabicyclo[3.1.0]hexane-3-carbonyl]-2-(2,2,2-trifluoro-1,1-dimethylethoxy)propyl]carbamate (600 mg, 677.99 umol, 74.20% yield, 75% purity) as a yellow oil. MS (ESI) m/z 664.5[M+H]⁺

Step 6: (1R,2S,5S)-3-[(2S,3R)-2-amino-3-(2,2,2-trifluoro-1,1-dimethyl-ethoxy)butanoyl]-N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide

To a solution of benzyl N-[(1S,2R)-1-[(1R,2S,5S)-2-[[(1S)-1-cyano-2-[(3R)-5,5-dimethyl-2-oxo-pyrrolidin-3-yl]ethyl]carbamoyl]-6,6-dimethyl-3-azabicyclo[3.1.0]hexane-3-carbonyl]-2-(2,2,2-trifluoro-1,1-dimethylethoxy)propyl]carbamate (580 mg, 637.91 umol, 73% purity, 1 eq) in IPA (5 mL) was added Pd(OH)₂ (72.17 mg, 102.78 umol, 20% purity, 1.61e-1 eq) under N₂. The suspension was degassed under vacuum and purged with H₂ several times. The mixture was stirred under H₂ (15 Psi) at 20° C. for 0.5 h. Upon completion, The reaction mixture was filtered and concentrated under reduced pressure to give (1R,2S,5S)-3-[(2S,3R)-2-amino-3-(2,2,2-trifluoro-1, l-dimethyl-ethoxy)butanoyl]-N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (400 mg, crude) as a white solid. MS (ESI) m/z 530.3 [M+H]⁺

Step 7: (1R,2S,5S)—N-[(1S)-1-cyano-2-[(3R)-5,5-dimethyl-2-oxo-pyrrolidin-3-yl]ethyl]-6,6-dimethyl-3-[(2S,3R)-2-[(2,2,2-trifluoroacetyl)amino]-3-(2,2,2-trifluoro-1,1-dimethyl-ethoxy)butanoyl]-3-azabicyclo[3.1.0]hexane-2-carboxamid

To a solution of dimethyl-2-oxo-pyrrolidin-3-yl]ethyl]-6,6-dimethyl-3-azabicyclo[3.1.0]hexane-2-carboxamide (400 mg, 755.30 umol, 1 eq) in methyl 2,2,2-trifluoroacetate (967.15 mg, 7.55 mmol, 761.54 uL, 10 eq) was added TEA (382.14 mg, 3.78 mmol, 525.64 uL, 5 eq). The mixture was stirred at 25° C. for 2 h. Upon completion, the mixture was concentrated in vacuo to give a residue. The residue was purified by prep-HPLC (column: Waters Xbridge Prep OBD C18 150*40 mm*10 um; mobile phase: [water(NH₄HCO₃)-ACN]; B %: 35%-65%, 8 min) to give (1R,2S,5S)—N-[(1S)-1-cyano-2-[(3R)-5,5-dimethyl-2-oxo-pyrrolidin-3-yl]ethyl]-6,6-dimethyl-3-[(2S,3R)-2-[(2,2,2-trifluoroacetyl)amino]-3-(2,2,2-trifluoro-1,1-dimethyl-ethoxy)butanoyl]-3-azabicyclo[3.1.0]hexane-2-carboxamide (143 mg, 228.58 umol, 30.26% yield, 100% purity) as a white solid. MS (ESI) m/z 626.3[M+H]⁺

¹H NMR (400 MHz, DMSO-d₆) δ=9.02 (d, J=8.6 Hz, 1H), 7.84 (s, 1H), 4.94 4.91 (m, 1H), 4.49 (d, J=9.1 Hz, 1H), 4.11 (s, 1H), 4.10-3.96 (m, 2H), 3.67 (d, J=10.5 Hz, 1H), 2.59-2.56 (m, 1H), 2.19-2.09 (m, 1H), 1.98 (dd, J=8.5, 12.2 Hz, 1H), 1.77-1.68 (m, 1H), 1.59-1.48 (m, 2H), 1.34 (d, J=7.6 Hz, 1H), 1.32 (s, 3H), 127 (s, 3H), 1.19 (s, 3H), 1.16-1.13 (m, 6H), 1.03 (s, 3H), 0.83 (s, 3H)

¹H NMR (400 MHz, METHANOL-d4) δ=5.00 (dd, J=5.1, 10.8 Hz, 1H), 4.62 (d, J=8.3 Hz, 1H), 4.23 (s, 1H), 4.17-4.13 (m, 1H), 4.08 (dd, J=5.6, 10.4 Hz, 1H), 3.83 (d, J=10.4 Hz, 1H), 2.86-2.78 (m, 1H), 2.33 (ddd, J=4.7, 10.9, 13.9 Hz, 1H), 2.19 (dd, J=8.6, 12.5 Hz, 1H), 1.91-1.83 (m, 1H), 1.67-1.62 (m, 2H), 1.43 (d, J=7.6 Hz, 1H), 1.37 (s, 3H), 1.33 (s, 3H), 1.30 (s, 3H), 1.25 (t, J=3.0 Hz, 6H), 1.09 (s, 3H), 0.93 (s, 3H)

Example 87: Synthesis of (1R,2S,5S)-3-[(2S,3R)-3-(1-cyanocyclopropoxy)-2-[(2,2,2-trifluoroacetyl)amino]butanoyl]-N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (Compound 334)

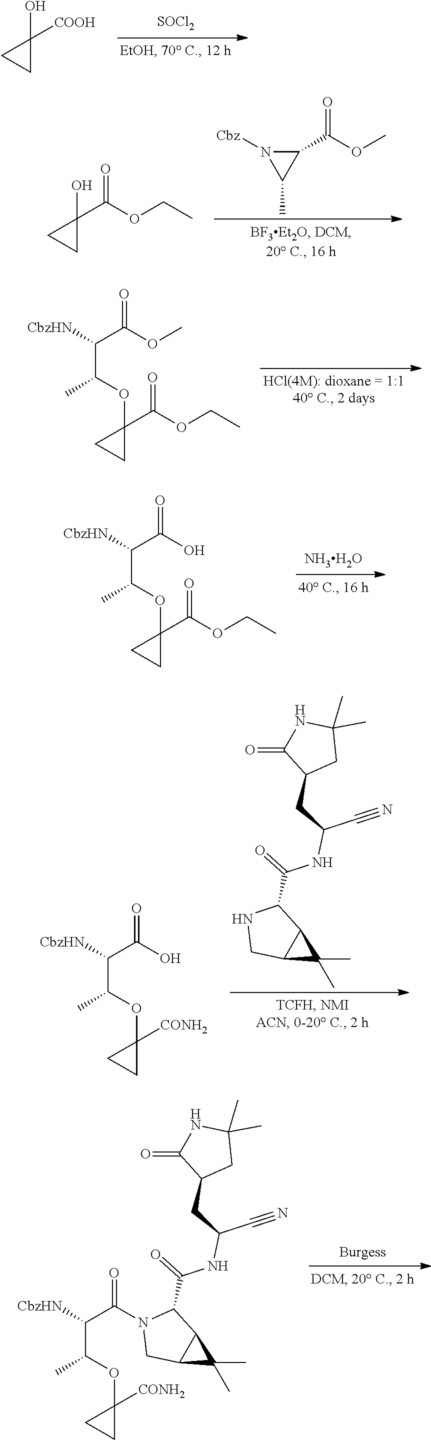

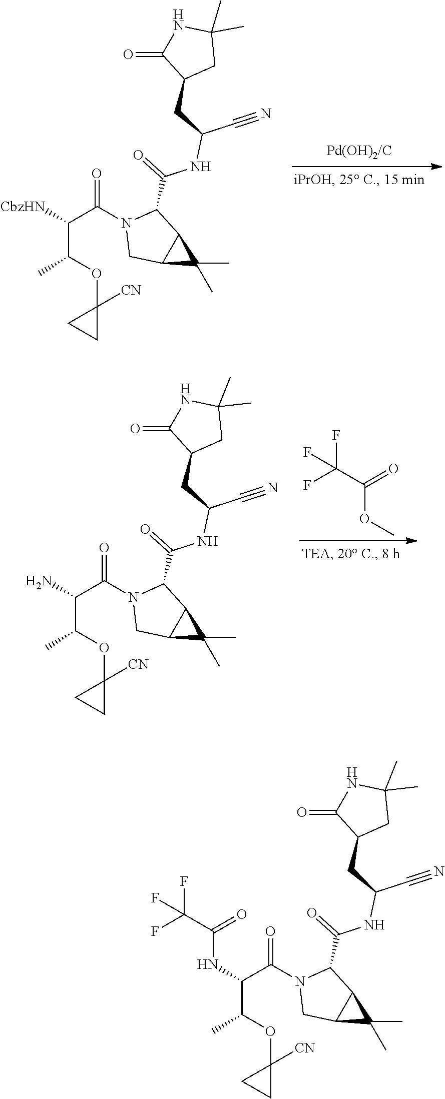

Step 1: ethyl 1-hydroxycyclopropanecarboxylate

To a solution of 1-hydroxycyclopropanecarboxylic acid (10 g, 97.95 mmol, 1 eq) in EtOH (150 mL) was added drop-wise SOCl₂ (5.83 g, 48.98 mmol, 3.55 mL, 0.5 eq), the mixture was stirred at 70° C. for 12 h. Upon completion, the reaction mixture was concentrated under reduced pressure to give ethyl 1-hydroxycyclopropanecarboxylate (11 g, crude) as a yellow oil. MS (ESI) m/z 131.1 [M+H]⁺.

Step 2: ethyl 1-[(1R,2S)-2-(benzyloxycarbonylamino)-3-methoxy-1-methyl-3-oxo-propoxy]cyclopropanecarboxylate

To a solution of O1-benzyl O2-methyl (2S,3S)-3-methylaziridine-1,2-dicarboxylate (7 g, 28.08 mmol, 1 eq) and ethyl 1-hydroxycyclopropanecarboxylate (9.14 g, 56.17 mmol, 812.07 uL, 80% purity, 2 eq) in DCM (150 mL) was added BF₃.Et₂₀ (848.04 mg, 2.81 mmol, 737.43 uL, 47% purity, 0.1 eq). The mixture was stirred at 20° C. for 16 h. Upon completion, the reaction mixture was quenched by addition of H₂O (200 mL) at 0° C., and then extracted with DCM (150 mL*2). The combined organic layer was washed with brine (100 mL), dried over Na₂SO₄, filtered and concentrated under reduced pressure to give a residue. The residue was purified by prep-HPLC (column: Xtimate C18 10 u 250 mm*80 mm; mobile phase: [water(NH₄HCO₃)-ACN]; B %: 35%-70%, 30 min) to give ethyl 1-[(1R,2S)-2-(benzyloxycarbonylamino)-3-methoxy-1-methyl-3-oxo-propoxy]cyclopropanecarboxylate (1.5 g, 3.95 mmol, 14.08% yield) as a yellow oil. MS (ESI) m/z 380.1 [M+H]⁺

Step 3: (2S,3R)-2-(benzyloxycarbonylamino)-3-(1-ethoxycarbonylcyclopropoxy)butanoic acid

A solution of ethyl 1-[(1R,2S)-2-(benzyloxycarbonylamino)-3-methoxy-1-methyl-3-oxo-propoxy]cyclopropanecarboxylate (750 mg, 1.58 mmol, 80% purity, 1 eq) (batch 2) in dioxane (15 mL) was added HCl (4 M, 15.00 mL, 37.94 eq), the mixture was stirred at 40° C. for 2 days. Upon completion, the reaction mixture was concentrated under reduced pressure to give a residue. The residue was purified by prep-HPLC (column: Phenomenex luna C18 (250*70 mm, 15 um); mobile phase: [water (HCl)-ACN]; B %: 30%-68%, 17 min) to give (2S,3R)-2-(benzyloxycarbonylamino)-3-(1-ethoxycarbonylcyclopropoxy)butanoic acid (0.6 g, 1.64 mmol, 51.92% yield) as a yellow oil. MS (ESI) m/z 364.2 [M−H]⁺

Step 4: (2S,3R)-2-(benzyloxycarbonylamino)-3-(1-carbamoylcyclopropoxy)butanoic acid

A mixture of (2S,3R)-2-(benzyloxycarbonylamino)-3-(1-ethoxycarbonylcyclopropoxy)butanoic acid (350 mg, 957.92 umol, 1 eq) in NH₃.H₂O (59.63 g, 612.50 mmol, 15 mL, 36% purity, 639.41 eq) was stirred at 40° C. for 16 h. Upon completion, the reaction mixture was concentrated under reduced pressure to remove 12 mL NH₃.H₂O. The residue was purified by prep-HPLC (column: Phenomenex Luna 80*30 mm 3 um; mobile phase: [water (HCl)-ACN]; B %: 10%-40%, 8 min) to give (2S,3R)-2-(benzyloxycarbonylamino)-3-(1-carbamoylcyclopropoxy)butanoic acid (220 mg, 654.10 umol, 68.28% yield) as a white solid. MS (ESI) m/z 335.1 [M−H]⁺.

Step 5: benzyl N-[(1S,2R)-2-(1-carbamoylcyclopropoxy)-1-[(1R,2S,5S)-2-[[(1S)-1-cyano-2-[(3R)-5,5-dimethyl-2-oxo-pyrrolidin-3-yl]ethyl]carbamoyl]-6,6-dimethyl-3-azabicyclo[3.1.0]hexane-3-carbonyl]propyl]carbamate

To a solution of (2S,3R)-2-(benzyloxycarbonylamino)-3-(1-carbamoylcyclopropoxy)butanoic acid (270 mg, 802.76 umol, 1 eq) and (1R,2S,5S)—N-[(1S)-1-cyano-2-[(3R)-5,5-dimethyl-2-oxo-pyrrolidin-3-yl]ethyl]-6,6-dimethyl-3-DY M1 26 9505 35.I azabicyclo[3.1.0]hexane-2-carboxamide (306.73 mg, 963.31 umol, 1.2 eq) in ACN (10 mL) was added NMI (263.63 mg, 3.21 mmol, 255.95 uL, 4 eq), then TCFH (270.29 mg, 963.31 umol, 1.2 eq) was added at 0° C. The mixture was stirred at 20° C. for 2 h. Upon completion, the reaction mixture was quenched by addition of H₂O (30 mL) at 0° C., and then extracted with EtOAc (20 mL*3). The combined organic layer was washed with brine (30 mL), dried over Na₂SO₄, filtered and concentrated under reduced pressure to give a residue. The residue was purified by prep-HPLC (column: Phenomenex C18 75*30 mm*3 um; mobile phase: [water(NH₄HCO₃)-ACN]; B %: 25%-55%, 8 min) to give benzyl N-[(1S,2R)-2-(1-carbamoylcyclopropoxy)-1-[(1R,2S,5S)-2-[[(1S)-1-cyano-2-[(3R)-5,5-dimethyl-2-oxo-pyrrolidin-3-yl]ethyl]carbamoyl]-6,6-dimethyl-3-azabicyclo[3.1.0]hexane-3-carbonyl]propyl]carbamate (300 mg, 471.15 umol, 58.69% yield) as a white solid. MS (ESI) m/z 637.3 [M+H]⁺.

Step 6: benzyl N-[(1S,2R)-2-(1-cyanocyclopropoxy)-1-[(1R,2S,5S)-2-[[(1S)-1-cyano-2-[(3R)-5,5-dimethyl-2-oxo-pyrrolidin-3-yl]ethyl]carbamoyl]-6,6-dimethyl-3-azabicyclo[3.1.0]hexane-3-carbonyl]propyl]carbamate

To a mixture of benzyl N-[(1S,2R)-2-(1-carbamoylcyclopropoxy)-1-[(1R,2S,5S)-2-[[(1S)-1-cyano-2-[(3R)-5,5-dimethyl-2-oxo-pyrrolidin-3-yl]ethyl]carbamoyl]-6,6-dimethyl-3-azabicyclo[3.1.0]hexane-3-carbonyl]propyl]carbamate (270 mg, 424.04 umol, 1 eq) in DCM (20 mL) was added BURGESS REAGENT (606.31 mg, 2.54 mmol, 6 eq), the mixture was stirred at 20° C. for 2 h. Upon completion, the reaction mixture was quenched by addition of H₂O (40 mL), and then extracted with DCM (20 mL*3). The combined organic layers were dried over Na₂SO₄, filtered and concentrated under reduced pressure to give benzyl N [(1S,2R)-2-(1-cyanocyclopropoxy)-1-[(1R,2S,5S)-2-[[(1S)-1-cyano-2-[(3R)-5,5-dimethyl-2-oxo-pyrrolidin-3-yl]ethyl]carbamoyl]-6,6-dimethyl-3-azabicyclo[3.1.0]hexane-3-carbonyl]propyl]carbamate (220 mg, crude) as a yellow solid. MS (ESI) m/z 619.4 [M+H]⁺.

Step 7: (1R,2S,5S)-3-[(2S,3R)-2-amino-3-(1-cyanocyclopropoxy)butanoyl]-N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide

A mixture of benzyl N-[(1S,2R)-2-(1-cyanocyclopropoxy)-1-[(1R,2S,5S)-2-[[(1S)-1-cyano-2-[(3R)-5,5-dimethyl-2-oxo-pyrrolidin-3-yl]ethyl]carbamoyl]-6,6-dimethyl-3-azabicyclo[3.1.0]hexane-3-carbonyl]propyl]carbamate (220 mg, 355.57 umol, 1 eq) in i-PrOH (10 mL) was added Pd(OH)₂ (113.21 mg, 806.09 umol, 2.27 eq) under H₂ (716.76 ug, 355.57 umol) (15 Psi), the mixture was stirred at 25° C. for 15 min. Upon completion, the reaction mixture was filtered and the filtrate was concentrated under reduced pressure to give (1R,2S,5S)-3-[(2S,3R)-2-amino-3-(1-cyanocyclopropoxy)butanoyl]-N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (150 mg, crude) as a yellow oil. MS (ESI) m/z 485.4 [M+H]⁺.

Step 8: (1R,2S,5S)—N-[(1S)-1-cyano-2-[(6R)-5-oxo-4-azaspiro[2.4]heptan-6-yl]ethyl]-6,6-dimethyl-3-[(2S,3R)-2-[(2,2,2-trifluoroacetyl)amino]-3-(2,2,2-trifluoroethoxy)butanoyl]-3-azabicyclo[3.1.0]hexane-2-carboxamide

To a mixture of (1R,2S,5S)-3-[(2S,3R)-2-amino-3-(1-cyanocyclopropoxy)butanoyl]-N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (150 mg, 309.54 umol, 1 eq) in methyl 2,2,2-trifluoroacetate (9.52 g, 74.39 mmol, 7.50 mL, 240.31 eq) was added TEA (156.61 mg, 1.55 mmol, 215.42 uL, 5 eq), the mixture was stirred at 20° C. for 8 h. Upon completion, the reaction mixture was concentrated under reduced pressure to give a residue. The residue was purified by prep-HPLC (column: Waters Xbridge Prep OBD C18 150*40 mm*10 um; mobile phase: [water(NH₄HCO₃)-ACN]; B %: 25%-55%, 8 min) to give (1R,2S,5S)-3-[(2S,3R)-3-(1-cyanocyclopropoxy)-2-[(2,2,2-trifluoroacetyl)amino]butanoyl]-N-[(1S)-1-cyano-2-[(3R)-5,5-dimethyl-2-oxo-pyrrolidin-3-yl]ethyl]-6,6-dimethyl-3-azabicyclo[3.1.0]hexane-2-carboxamide (64 mg, 110.23 umol, 35.61% yield, 100% purity) as a white solid. MS (ESI) m/z 581.3 [M+H]⁺.

¹H NMR (400 MHz, DMSO-d₆) δ=10.22-9.36 (m, 1H), 9.04 (d, J=8.6 Hz, 1H), 7.82 (s, 1H), 4.99-4.89 (m, 1H), 4.43 (d, J=9.2 Hz, 1H), 4.11 (s, 1H), 4.04-3.91 (m, 2H), 3.73 (d, J=10.5 Hz, 1H), 2.58 (d, J=3.7 Hz, 1H), 2.21-2.10 (m, 1H), 2.04-1.93 (m, 1H), 1.78-1.67 (m, 1H), 1.62-1.51 (m, 2H), 1.49-1.42 (m, 1H), 1.41-1.28 (m, 5H), 1.26 1.19 (m, 4H), 1.15-1.08 (m, 4H), 1.03 (s, 3H), 0.84 (s, 3H)

Example 88: Synthesis of (1R,2S,5S)—N-[(1S)-1-cyan-2-[(6R)-5-oxo-4-azaspiro[2.4]heptan-6-yl]ethyl]-6,6-dimethyl-3-[(2S,3R)-2-[(2,2,2-trifluoroacetyl)amino]-3-(2,2,2-trifluoro-1,1-dimethyl-ethoxy)butanoyl]-3-azabicyclo[3.1.0]hexane-2-carboxamide (Compound 358)

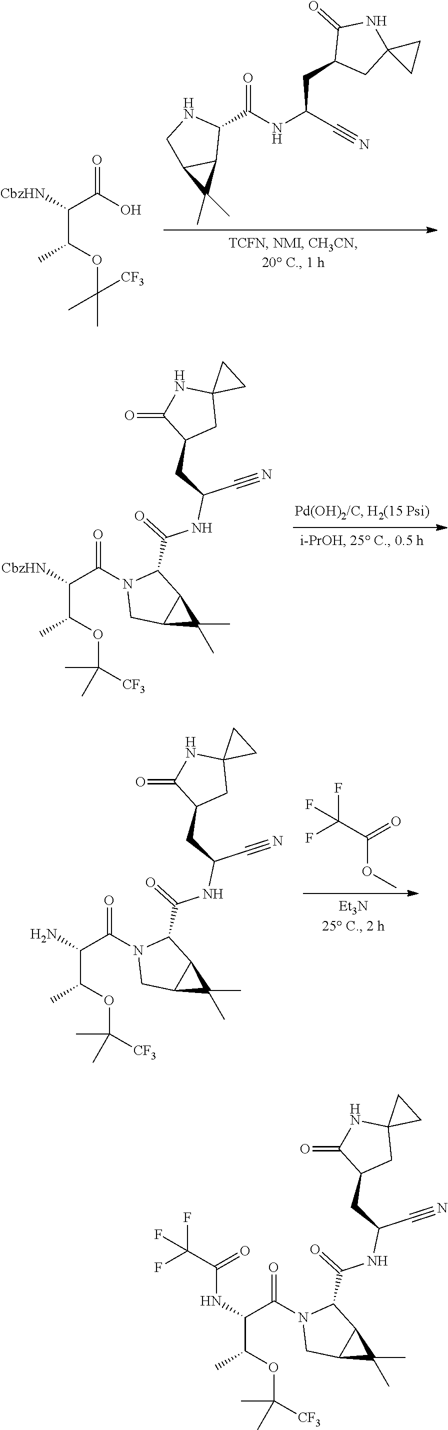

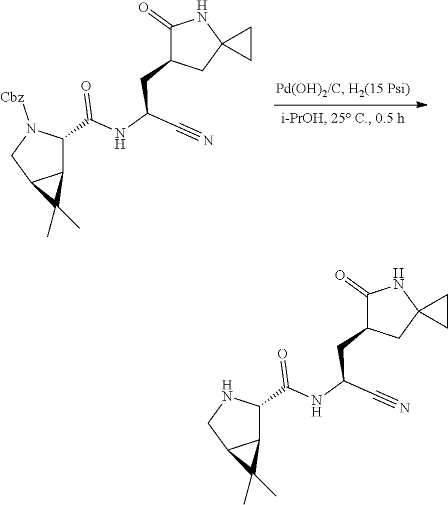 [SMILES: CC1(C)[C@@H]2[C@@H](C(=O)N[C@H](C#N)C[C@@H]3CC4(CC4)NC3=O)N(C(=O)OCc3ccccc3)C[C@@H]21 ; CC1(C)[C@@H]2[C@@H](C(=O)N[C@H](C#N)C[C@@H]3CC4(CC4)NC3=O)NC[C@@H]21]

Step 1: (1R,2S,5S)—N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide

To a solution of benzyl (1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carboxylate (830 mg, 1.84 mmol, 1 eq) in IPA (10 mL) was added Pd(OH)₂ (815.00 mg, 1.16 mmol, 20% purity, 0.63 eq) under N₂. The suspension was degassed under vacuum and purged with H₂ several times. The mixture was stirred under H₂ (15 psi) at 20° C. for 0.5 h. Upon completion, the reaction mixture was filtered and concentrated under reduced pressure to give (1R,2S,5S)—N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (500 mg, crude) as a white solid, which was used for next step without further purification. MS (ESI) m/z 317.3 [M+H]⁺.

Step 2: benzyl N-[(1S,2R)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2-(2,2,2-trifluoro-1,1-dimethylethoxy)propyl]carbamate

To a solution of (2S,3R)-2-(benzyloxycarbonylamino)-3-(2,2,2-trifluoro-1,1-dimethyl-ethoxy)butanoic acid (370 mg, 1.02 mmol, 1 eq), (1R,2S,5S)—N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (322.21 mg, 1.02 mmol, 1 eq) in ACN (5 mL) was added 1-methylimidazole(250.82 mg, 3.06 mmol, 243.52 uL, 3 eq) and 1-[chloro(pyrrolidin-1-ium-1-ylidene)methyl]pyrrolidine; hexafluorophosphate (677.53 mg, 2.04 mmol, 2 eq). The mixture was stirred at 20° C. for 1 h. Upon completion, the reaction mixture was diluted with water (5 mL) and extected with ethyl acetate (5 mL*2). The organic phase was washed with brine (5 mL), dried over anhydrous magnesium sulfate, filtered, concentrated under reduced pressure to afford a residue, which was purified by column chromatography (SiO₂, Petroleum ether/Ethyl acetate=1011 to 011) to give benzyl N-[(1S,2R)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2-(2,2,2-trifluoro-1,1-dimethylethoxy)propyl]carbamate (600 mg, 725.39 umol, 71.23% yield, 80% purity) as a yellow oil. MS (ESI) m/z 662.3 [M+H]⁺

Step 3: (1R,2S,5S)-3-[(2S,3R)-2-amino-3-(2,2,2-trifluoro-1,1-dimethyl-ethoxy)butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide

To a solution of benzyl N-[(1S,2R)-1-[(1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carbonyl]-2-(2,2,2-trifluoro-1,1-dimethylethoxy)propyl]carbamate (600 mg, 725.39 umol, 80% purity, 1 eq) in IPA (15 mL) was added Pd(OH)₂ (98.44 mg, 140.19 umol, 20% purity, 1.93e-1 eq) under N₂. The suspension was degassed under vacuum and purged with H₂ several times. The mixture was stirred under H₂ (15 psi) at 20° C. for 0.5 h. Upon completion, The reaction mixture was filtered and concentrated under reduced pressure to give (1R,2S,5S)-3-[(2S,3R)-2-amino-3-(2,2,2-trifluoro-1,1-dimethyl-ethoxy)butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (400 mg, crude) as a white solid. MS (ESI) m/z 528.3 [M+H]⁺

Step 4: (1R,2S,5S)—N-[(1S)-1-cyano-2-[(6R)-5-oxo-4-azaspiro[2.4]heptan-6-yl]ethyl]-6,6-dimethyl-3-[(2S,3R)-2-[(2,2,2-trifluoroacetyl)amino]-3-(2,2,2-trifluoro-1,1-dimethyl-ethoxy)butanoyl]-3-azabicyclo[3.1.0]hexane-2-carboxamide

To a solution of (1R,2S,5S)-3-[(2S,3R)-2-amino-3-(2,2,2-trifluoro-1,1-dimethyl-ethoxy)butanoyl]-N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (400 mg, 758.18 umol, 1 eq) in methyl 2,2,2-trifluoroacetate (970.85 mg, 7.58 mmol, 764.45 uL, 10 eq) was added TEA (383.60 mg, 3.79 mmol, 527.65 uL, 5 eq). The mixture was stirred at 20° C. for 2 h. Upon completion, the mixture was concentrated in vacuo to give a residue. The residue was purified by prep-HPLC (column: Waters Xbridge Prep OBD C18 150*40 mm*10 um; mobile phase: [water(NH₄HCO₃)-ACN]; B %: 35%-65%, 8 min) to give (1R,2S,5S)—N-[(1S)-1-cyano-2-[(6R)-5-oxo-4-azaspiro[2.4]heptan-6-yl]ethyl]-6,6-dimethyl-3-[(2S,3R)-2-[(2,2,2-trifluoroacetyl)amino]-3-(2,2,2-trifluoro-1,1-dimethyl-ethoxy)butanoyl]-3-azabicyclo[3.1.0]hexane-2-carboxamide (118 mg, 189.23 umol, 24.96% yield, 100% purity) as a white solid. MS (ESI) m/z 624.2[M+H]⁺

¹H NMR (400 MHz, DMSO-d₆) δ=9.98 (br d, J=8.6 Hz, 1H), 9.06-9.02 (m, 1H), 7.78 (s, 1H), 4.95 (ddd, J=5.8, 8.4, 10.2 Hz, 1H), 4.48 (br t, J=8.5 Hz, 1H), 4.12-4.07 (m, 2H), 3.98 (dd, J=5.4, 10.4 Hz, 1H), 3.63 (d, J=10.5 Hz, 1H), 2.65 (dq, J=4.9, 9.5 Hz, 1H), 2.23-2.17 (m, 1H), 2.03-1.79 (m, 3H), 1.57 (dd, J=5.4, 7.5 Hz, 1H), 1.35-1.33 (m, 3H), 1.29 (s, 3H), 1.14 (d, J=6.1 Hz, 3H), 1.04-1.01 (m, 3H), 1.00-0.93 (m, 1H), 0.83 (s, 3H), 0.78-0.71 (m, 1H), 0.61-0.51 (m, 3H)

Example 89: Synthesis of (1R,2S,5S)—N-[(1S)-1-cyan-2-[(6R)-5-oxo-4-azaspiro[2.4]heptan-6-yl]ethyl]-3-[(2S,3R)-3-(3,3-difluorocyclobutoxy)-2-[(2,2,2-trifluoroacetyl)amino]butanoyl]-6,6-dimethyl-3-azabicyclo[3.1.0]hexane-2-carboxamide (Compound 430)

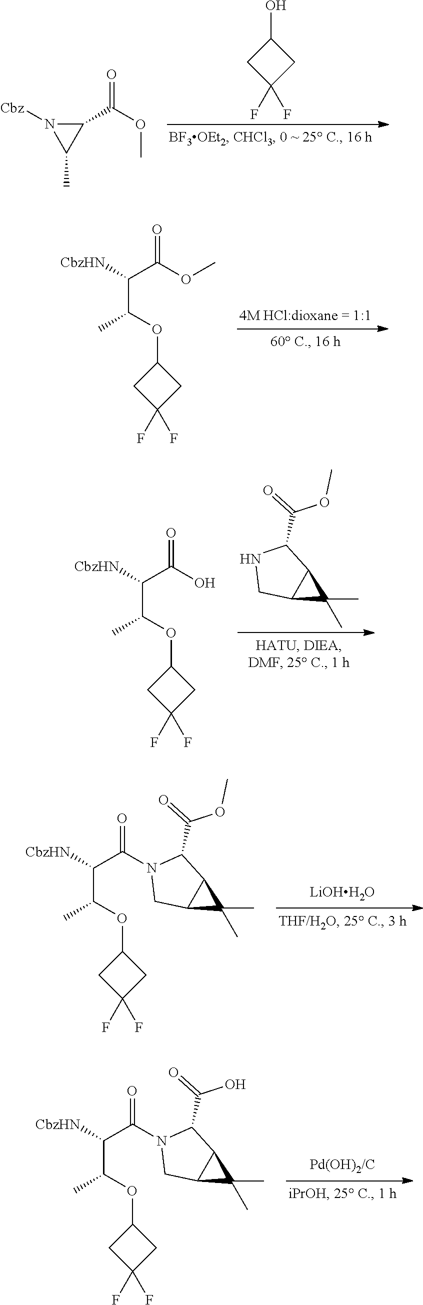

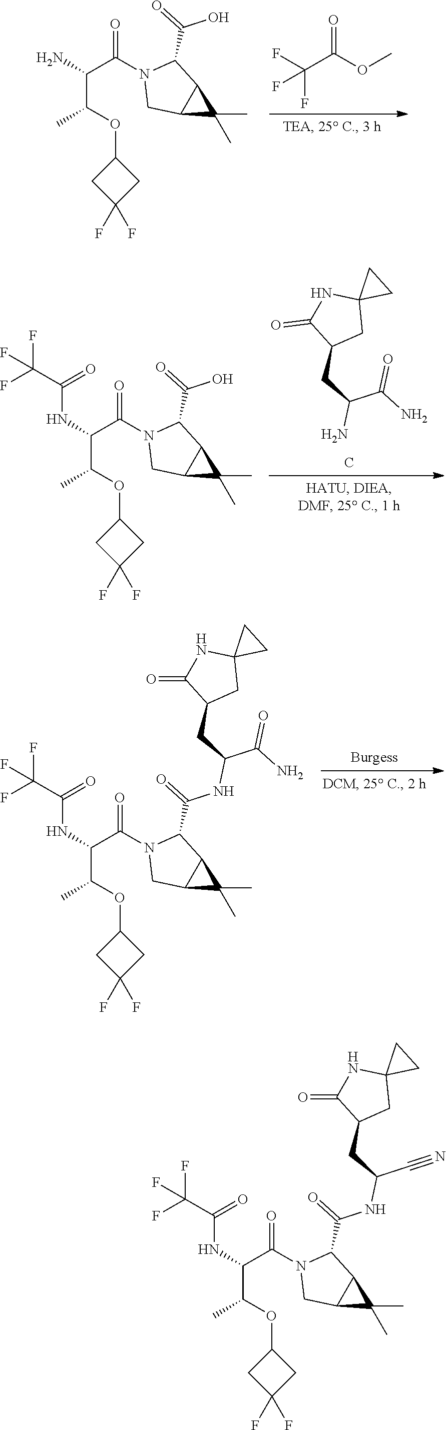

Step 1: methyl (2S,3R)-2-(benzyloxycarbonylamino)-3-(3,3-difluorocyclobutoxy)butanoate

To a mixture of O1-benzyl O2-methyl (2S,3S)-3-methylaziridine-1,2-dicarboxylate (3 g, 12.04 mmol, 1 eq) and 3,3-difluorocyclobutanol (1.56 g, 14.44 mmol, 1.2 eq) in CHCl₃ (45 mL) was added BF₃.Et₂₀ (363.44 mg, 1.20 mmol, 316.04 uL, 47% purity, 0.1 eq) at 0° C., the mixture was stirred at 25° C. for 16 h. Upon completion, the reaction mixture was quenched by addition of H₂O 40 mL at 0° C., and then extracted with DCM 120 mL (40 mL*3). The combined organic layer was washed with brine 60 mL (60 mL*1), dried over Na₂SO₄, filtered and concentrated under reduced pressure to give the crude product. The crude was purified by column chromatography (SiO₂, Petroleum ether:Ethyl acetate=50:1 to 4:1) to give methyl (2S,3R)-2-(benzyloxycarbonylamino)-3-(3,3-difluorocyclobutoxy)butanoate (2.3 g, 5.47 mmol, 45.46% yield, 85% purity) as a colorless oil. MS (ESI) m/z 358.3 [M+H]⁺

Step 2: (2S,3R)-2-(benzyloxycarbonylamino)-3-(3,3-difluorocyclobutoxy)butanoic acid

A mixture of methyl (2S,3R)-2-(benzyloxycarbonylamino)-3-(3,3-difluorocyclobutoxy)butanoate (2.3 g, 6.44 mmol, 1 eq) in HCl (4 M, 115 mL, 71.47 eq) and dioxane (115 mL) was stirred at 60° C. for 16 h. Upon completion, the reaction mixture was diluted with H₂O 200 mL and extracted with DCM 600 mL (200 mL*3). The combined organic layer was washed with brine 300 mL (300 mL*1), dried over Na₂SO₄, filtered and concentrated under reduced pressure to give (2S,3R)-2-(benzyloxycarbonylamino)-3-(3,3-difluorocyclobutoxy)butanoic acid (2.2 g, crude) as a colorless oil. MS (ESI) m/z 344.3 [M+H]⁺

Step 3: methyl(1R,2S,5S)-3-[(2S,3R)-2-(benzyloxycarbonylamino)-3-(3,3-difluorocyclobutoxy)butanoyl]-6,6-dimethyl-3-azabicyclo[3.1.0]hexane-2-carboxylate

To a mixture of (2S,3R)-2-(benzyloxycarbonylamino)-3-(3,3-difluorocyclobutoxy)butanoic acid (2.2 g, 6.41 mmol, 1 eq) in DMF (60 mL) was added HATU (3.65 g, 9.61 mmol, 1.5 eq), DIEA (828.19 mg, 6.41 mmol, 1.12 mL, 1 eq) and methyl (1R,2S,5S)-6,6-dimethyl-3-azabicyclo [3.1.0]hexane-2-carboxylate (1.32 g, 6.41 mmol, 1 eq, HCl) in turn. The mixture was stirred at 25° C. for 1 h. Upon completion, the reaction mixture was diluted with H₂O 60 mL and extracted with EA 180 mL (60 mL*3). The combined organic layer was washed with brine 450 mL (90 mL*5), dried over Na₂SO₄, filtered and concentrated under reduced pressure to give the crude product. The crude was purified by column chromatography (SiO₂, petroleum ether:ethyl acetate=20:1 to 3:1) to give methyl (1R,2S,5S)-3-[(2S,3R)-2-(benzyloxycarbonylamino)-3-(3,3-difluorocyclobutoxy)butanoyl]-6,6-dimethyl-3-azabicyclo[3.1.0]hexane-2-carboxylate (2.5 g, 4.30 mmol, 67.06% yield, 85% purity) as a colorless oil. MS (ESI) m/z 495.4 [M+H]⁺

Step 4: (1R,2S,5S)-3-[(2S,3R)-2-(benzyloxycarbonylamino)-3-(3,3-difluorocyclobutoxy)butanoyl]-6,6-dimethyl-3-azabicyclo[3.1.0]hexane-2-carboxylic acid

To a mixture of methyl (1R,2S,5S)-3-[(2S,3R)-2-(benzyloxycarbonylamino)-3-(3,3-difluorocyclobutoxy)butanoyl]-6,6-dimethyl-3-azabicyclo[3.1.0]hexane-2-carboxylate (2.5 g, 5.06 mmol, 1 eq) in THE (75 mL) and H₂O (25 mL) was added LiOH.H₂O (424.28 mg, 10.11 mmol, 2 eq). The mixture was stirred at 25° C. for 3 h. Upon completion, the reaction mixture was adjusted pH=2 by 1M HCl, and then extracted with DCM 450 mL (150 mL*3). The combined organic layer was washed with brine 400 mL (200 mL*2), dried over Na₂SO₄, filtered and concentrated under reduced pressure to give (1R,2S,5S)-3-[(2S,3R)-2-(benzyloxycarbonylamino)-3-(3,3-difluorocyclobutoxy)butanoyl]-6,6-dimethyl-3-azabicyclo[3.1.0]hexane-2-carboxylic acid (2.3 g, crude) as a white solid. MS (ESI) m/z 481.3 [M+H]⁺

Step 5: (1R,2S,5S)-3-[(2S,3R)-2-amino-3-(3,3-difluorocyclobutoxy)butanoyl]-6,6-dimethyl-3-azabicyclo[3.1.0]hexane-2-carboxylic acid

To a mixture of (1R,2S,5S)-3-[(2S,3R)-2-(benzyloxycarbonylamino)-3-(3,3-difluorocyclobutoxy) butanoyl]-6,6-dimethyl-3-azabicyclo[3.1.0]hexane-2-carboxylic acid (2.3 g, 4.79 mmol, 1 eq) in i-PrOH (12 mL) was added Pd(OH)₂ (2.3 g, 16.38 mmol, 3.42 eq). The mixture was stirred at 25° C. for 1 h under H₂ (9.65 mg, 4.79 mmol, 1 eq) at 15 Psi. Upon completion, the reaction mixture was filtered and concentrated under reduced pressure to give (1R,2S,5S)-3-[(2S,3R)-2-amino-3-(3,3-difluoro cyclobutoxy)butanoyl]-6,6-dimethyl-3-azabicyclo[3.1.0]hexane-2-carboxylic acid (1.1 g, crude) as a white solid. MS (ESI) m/z 347.4 [M+H]⁺

Step 6: (1R,2S,5S)-3-[(2S,3R)-3-(3,3-difluorocyclobutoxy)-2-[(2,2,2-trifluoroacetyl)amino]butanoyl]-6,6-dimethyl-3-azabicyclo[3.1.0]hexane-2-carboxylic acid

To a mixture of (1R,2S,5S)-3-[(2S,3R)-2-amino-3-(3,3-difluorocyclobutoxy)butanoyl]-6,6-dimethyl-3-azabicyclo[3.1.0]hexane-2-carboxylic acid (1.1 g, 3.18 mmol, 1 eq) in methyl 2,2,2-trifluoroacetate (19.05 g, 148.77 mmol, 15 mL, 46.84 eq) was added TEA (964.07 mg, 9.53 mmol, 1.33 mL, 3 eq). The mixture was stirred at 25° C. for 3 h. Upon completion, the reaction mixture was concentrated under reduced pressure to give (1R,2S,5S)-3-[(2S,3R)-3-(3,3-difluorocyclobutoxy)-2-[(2,2,2-trifluoroacetyl)amino] butanoyl]-6,6-dimethyl-3-azabicyclo[3.1.0]hexane-2-carboxylic acid (1.4 g, crude) as a yellow oil. MS (ESI) m/z 443.2 [M+H]⁺

Step 7: (1R,2S,5S)—N-[(1S)-2-amino-2-oxo-1-[[(6R)-5-oxo-4-azaspiro[2.4]heptan-6-yl]methyl]ethyl]-3-[(2S,3R)-3-(3,3-difluorocyclobutoxy)-2-[(2,2,2-trifluoroacetyl)amino]butanoyl]-6,6-dimethyl-3-azabicyclo[3.1.0]hexane-2-carboxamide

To a mixture of (1R,2S,5S)-3-[(2S,3R)-3-(3,3-difluorocyclobutoxy)-2-[(2,2,2-trifluoroacetyl)amino] butanoyl]-6,6-dimethyl-3-azabicyclo[3.1.0]hexane-2-carboxylic acid (0.7 g, 1.58 mmol, 1 eq) in DMF (20 mL) was added HATU (902.49 mg, 2.37 mmol, 1.5 eq), DIEA (613.53 mg, 4.75 mmol, 826.86 uL, 3 eq) and (2S)-2-amino-3-[(6R)-5-oxo-4-azaspiro[2.4]heptan-6-yl]propanamide (369.79 mg, 1.58 mmol, 1 eq, HCl) in turn. The mixture was stirred at 25° C. for 1 h. Upon completion, the reaction mixture was diluted with H₂O b mL and extracted with EA 120 mL (40 mL*3). The combined organic layer was washed with brine 120 mL (60 mL*2), dried over Na₂SO₄, filtered and concentrated under reduced pressure to give the crude product. The crude was purified by column chromatography (SiO₂, Petroleum ether:Ethyl acetate=10:1 to EA:MeOH=30:1) to give (1R,2S,5S)—N-[(1S)-2-amino-2-oxo-1-[[(6R)-5-oxo-4-azaspiro[2.4]heptan-6-yl]methyl]ethyl]-3-[(2S,3R)-3-(3,3-difluorocyclobutoxy)-2-[(2,2,2-trifluoroacetyl)amino]butanoyl]-6,6-dimethyl-3-azabicyclo[3.1.0]hexane-2-carboxamide (450 mg, 579.16 umol, 36.60% yield, 80% purity) as a yellow solid. MS (ESI) m/z 622.3 [M+H]⁺

Step 8: (1R,2S,5S)—N-[(1S)-1-cyano-2-[(6R)-5-oxo-4-azaspiro[2.4]heptan-6-yl]ethyl]-3-[(2S,3R)-3-(3,3-difluorocyclobutoxy)-2-[(2,2,2-trifluoroacetyl)amino]butanoyl]-6,6-dimethyl-3-azabicyclo[3.1.0]hexane-2-carboxamide

To a mixture of (1R,2S,5S)—N-[(1S)-2-amino-2-oxo-1-[[(6R)-5-oxo-4-azaspiro[2.4]heptan-6-yl]methyl]ethyl]-3-[(2S,3R)-3-(3,3-difluorocyclobutoxy)-2-[(2,2,2-trifluoroacetyl)amino]butanoyl]-6,6-dimethyl-3-azabicyclo[3.1.0]hexane-2-carboxamide (450 mg, 723.94 umol, 1 eq) in DCM (10 mL) was added burgess reagent (517.56 mg, 2.17 mmol, 3 eq). The mixture was stirred at 25° C. for 2 h. Upon completion, the reaction mixture was quenched by addition of H₂O 2 mL at 25° C., and then concentrated under reduced pressure to give the crude product. The crude was purified by prep-HPLC (neutral condition; column: Waters Xbridge Prep OBD C18 150*40 mm*10 um; mobile phase: [water(NH₄HCO₃)-ACN]; B %: 35%-55%, 8 min) to give (1R,2S,5S)—N-[(1S)-1-cyano-2-[(6R)-5-oxo-4-azaspiro[2.4]heptan-6-yl]ethyl]-3-[(2S,3R)-3-(3,3-difluorocyclobutoxy)-2-[(2,2,2-trifluoroacetyl)amino]butanoyl]-6,6-dimethyl-3-azabicyclo[3.1.0]hexane-2-carboxamide (0.15 g, 248.52 umol, 34.33% yield, 100% purity) as a white solid. MS (ESI) m/z 604.3 [M+H]⁺

¹H NMR (400 MHz, DMSO-d₆) δ=9.96 (br s, 1H), 9.04 (d, J=8.3 Hz, 1H), 7.77 (s, 1H), 4.93 (ddd, J=5.7, 8.5, 10.0 Hz, 1H), 4.42 (br d, J=8.6 Hz, 1H), 4.19-4.06 (m, 2H), 3.93 (dd, J=5.4, 10.4 Hz, 1H), 3.82-3.66 (m, 2H), 2.92-2.72 (m, 2H), 2.70-2.54 (m, 2H), 2.44-2.31 (m, 1H), 2.17 (ddd, J=4.6, 10.3, 13.6 Hz, 1H), 2.04-1.76 (m, 3H), 1.57 (dd, J=5.5, 7.4 Hz, 1H), 1.33 (d, J=7.6 Hz, 1H), 1.15 (d, J=6.2 Hz, 3H), 1.06-1.01 (m, 3H), 0.85 (s, 3H), 0.79-0.72 (m, 1H), 0.64-0.49 (m, 3H)

Example 90: Synthesis of (1R, 2S, 5S)-3-((2S, 3R)-3-(tert-butoxy)-2-(2,2,2-trifluoroacetamido)butanoyl)-N—((S)-1-cyano-2-((S)-2-oxopiperidin-3-yl)ethyl)-6,6-dimethyl-3-azabicyclo[3.1.0]hexane-2-carboxamide (Compound 431)

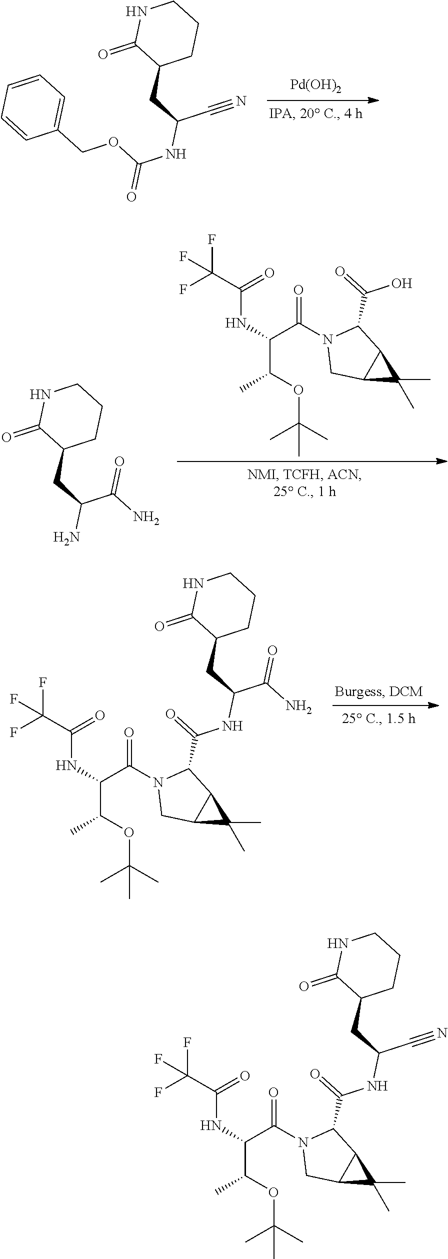

Step 1: (S)-2-amino-3-((S)-2-oxopiperidin-3-yl)propanamide

To a solution of benzyl N-[(1S)-1-cyano-2-[(3S)-2-oxo-3-piperidyl]ethyl]carbamate (800 mg, 2.65 mmol, 1 eq) in IPA (8 mL) was added Pd(OH)₂ (800 mg, 1.14 mmol, 20% purity, 4.29e⁻¹ eq) under N₂. The suspension was degassed under vacuum and purged with H₂ several times. The mixture was stirred under H₂ (15 psi) at 20° C. for 4 hours. Upon completion, the reaction mixture was filtered and concentrated under reduced pressure to give a residue to give (S)-2-amino-3-((S)-2-oxopiperidin-3-yl) propanamide (640 mg, crude) as a yellow solid.

Step 2: (1R,2S,5S)-N—((S)-1-amino-1-oxo-3-((S)-2-oxopiperidin-3-yl)propan-2-yl)-3-((2S,3R)-3-(tert-butoxy)-2-(2,2,2-trifluoroacetamido)butanoyl)-6,6-dimethyl-3-azabicyclo[3.1.0]hexane-2-carboxamide

To a solution of (2S)-2-amino-3-[(3S)-2-oxo-3-piperidyl]propanamide (181.41 mg, 685.58 umol, 70% purity, 1 eq) in ACN (5 mL) was added (1R,2S,5S)-3-[(2S,3R)-3-tert-butoxy-2-[(2,2,2-trifluoroacetyl)amino]butanoyl]-6,6-dimethyl-3-azabicyclo[3.1.0]hexane-2-carboxylic acid (280 mg, 685.58 umol, 1 eq) and [chloro(dimethylamino)methylene]-dimethyl-ammonium; hexafluorophosphate (384.72 mg, 1.37 mmol, 2 eq) and 1-methylimidazole (168.86 mg, 2.06 mmol, 163.94 uL, 3 eq) in turn. Then the mixture was stirred for 1 h at 25° C. Upon completion, the resulting solution was poured into H₂O (3 mL), and then extracted with DCM (20 mL×3). The combined organic phase was washed brine (30 mL*3), dried over Na₂SO₄, filtered and concentrated to give a crude product. The residue was purified by column chromatography (SiO₂, Petroleum ether:Ethyl acetate=5:1 to 0:1) to give (1R,2S,5S)-N—((S)-1-amino-1-oxo-3-((S)-2-oxopiperidin-3-yl)propan-2-yl)-3-((2S,3R)-3-(tert-butoxy)-2-(2,2,2-trifluoroacetamido)butanoyl)-6,6-dimethyl-3-azabicyclo[3.1.0]hexane-2-carboxamide (360 mg, 625.41 umol, 91.22% yield) as a light yellow oil.

Step 3: (1R,2S,5S)-3-((2S,3R)-3-(tert-butoxy)-2-(2,2,2-trifluoroacetamido)butanoyl)-N—((S)-1-cyano-2-((S)-2-oxopiperidin-3-yl)ethyl)-6,6-dimethyl-3-azabicyclo[3.1.0]hexane-2-carboxamide

To a solution of (1R,2S,5S)—N-[(1S)-2-amino-2-oxo-1-[[(3S)-2-oxo-3-piperidyl]methyl]ethyl]-3-[(2S,3R)-3-tert-butoxy-2-[(2,2,2-trifluoroacetyl)amino]butanoyl]-6,6-dimethyl-3-azabicyclo[3.1.0]hexane-2-carboxamide (330 mg, 573.30 umol, 1 eq) in DCM (5 mL) was added burgess reagent (409.87 mg, 1.72 mmol, 3 eq). The mixture was stirred for 1.5 h at 25° C. Upon completion, the reaction was quenched with H₂O (0.2 mL), and was blowed to dryness with N₂ to afford a residue, which was purified by prep-HPLC column: Waters Xbridge BEH C18 250*50 mm*10 um; mobile phase: [water(NH₄HCO₃)-ACN]; B %: 30%-60%, 10 min to give (1R,2S,5S)-3-((2S,3R)-3-(tert-butoxy)-2-(2,2,2-trifluoroacetamido)butanoyl)-N—((S)-1-cyano-2-((S)-2-oxopiperidin-3-yl)ethyl)-6,6-dimethyl-3-azabicyclo[3.1.0]hexane-2-carboxamide (84.72 mg, 151.94 umol, 26.50% yield, 100% purity) as a white solid. MS (ESI) m/z 502.2[M+H]⁺.

¹H NMR (400 MHz, DMSO-d₆) δ=9.80-9.61 (m, 1H), 8.97 (d, J=8.3 Hz, 1H), 7.53 (br s, 1H), 5.07-4.97 (m, 1H), 4.44-4.26 (m, 1H), 4.13 (s, 1H), 4.02-3.87 (m, 2H), 3.65 (d, J=10.5 Hz, 1H), 3.10 (br d, J=2.8 Hz, 2H), 2.35-2.25 (m, 2H), 1.91-1.82 (m, 1H), 1.77-1.70 (m, 2H), 1.62-1.54 (m, 2H), 1.34-1.31 (m, 1H), 1.16 (s, 1H), 1.11 (s, 9H), 1.08 (d, J=6.0 Hz, 3H), 1.05-1.02 (m, 3H), 1.00-0.97 (m, 1H), 0.83 (s, 3H)

Example 91: Synthesis of (1R,5S)—N-[(1S)-1-cyano-2-(6,6-dimethyl-2-oxo-3-piperidyl)ethyl]-3-[(2S)-3,3-dimethyl-2-[(2,2,2-trifluoroacetyl)amino]butanoyl]-6,6-dimethyl-3-azabicyclo[3.1.0]hexane-2-carboxamide (Compound 361)

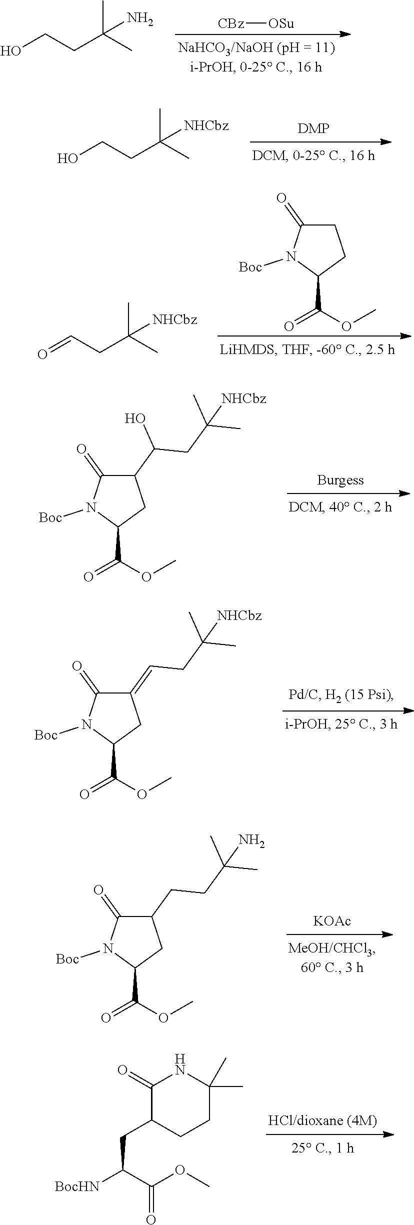

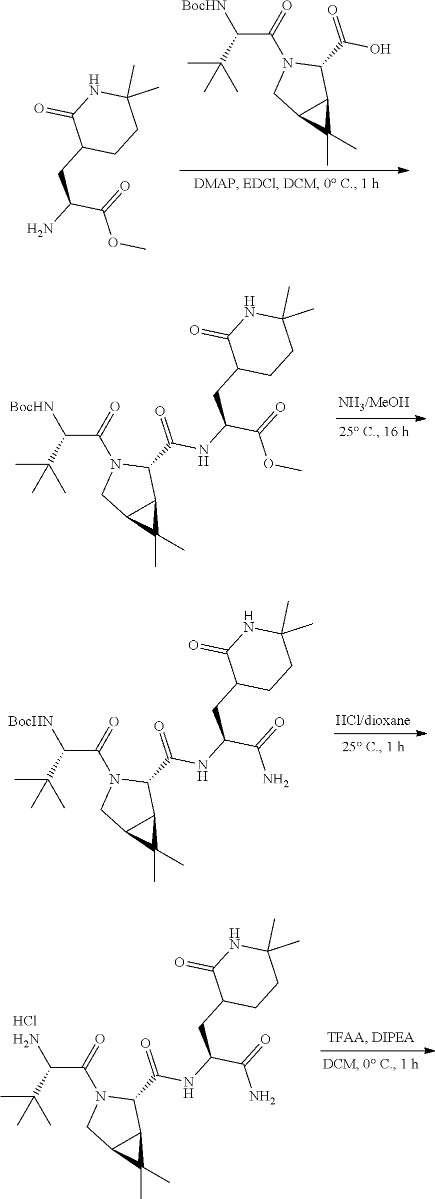

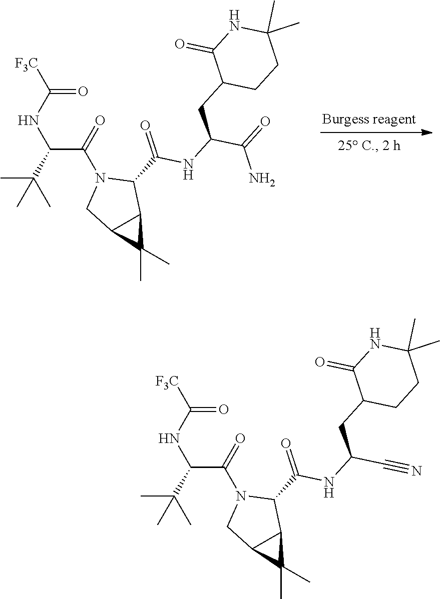

Step 1: benzyl N-(3-hydroxy-1,1-dimethyl propyl)carbamate

750 mL of a buffer-pH=11(Saturated NaHCO₃(756.00 g, 9.00 mol, 350 mL, 18.57 eq), adjusted with NaOH aq. (4 M, 350 mL, 2.89 eq) to pH=11) was added to a solution of 3-amino-3-methyl-butan-1-ol (50 g, 484.67 mmol, 1 eq) in IPA (750 mL). The reaction mixture was cooled to 0° C. and benzyl (2,5-dioxopyrrolidin-1-yl) carbonate (120.79 g, 484.67 mmol, 1 eq) was added. The reaction mixture was stirred at 25° C. for 16 h. Upon completion, the reaction mixture was concentrated under reduced pressure, and then diluted with water (2000 mL) and extracted with ethyl acetate (1000 mL*3). The combined organic layers were washed with brine (500 mL), dried over Na₂SO₄, filtered and concentrated under reduced pressure to give a residue. The residue was purified by column chromatography (SiO₂, Petroleum ether:Ethyl acetate=10:1 to 1:1) to give benzyl N-(3-hydroxy-1,1-dimethyl-propyl)carbamate (200 g, 842.84 mmol, 86.95% yield) as a colorless oil.

Step 2: benzyl N-(1,1-dimethyl-3-oxo propyl)carbamate

To a solution of benzyl N-(3-hydroxy-1,1-dimethyl-propyl)carbamate (90 g, 379.28 mmol, 1 eq) in DCM (1 L) was added DMP (193.04 g, 455.13 mmol, 140.90 mL, 1.2 eq) at 0° C. The reaction mixture was stirred at 25° C. for 16 h. Upon completion, the reaction mixture was diluted with water (1000 mL) and extracted with DCM (500 mL*3). The combined organic layers were washed with sat. NaHCO₃(1000 mL), dried over Na₂SO₄, filtered and concentrated under reduced pressure to give a residue. The residue was purified by column chromatography (SiO₂, Petroleum ether:Ethyl acetate=10:1 to 1:1) to give benzyl N-(1,1-dimethyl-3-oxo-propyl)carbamate (110 g, 420.78 mmol, 55.47% yield, 90% purity) as a colorless oil.

Step 3: O1-tert-butyl O2-methyl (2S)-4-[3-(benzyloxycarbonylamino)-1-hydroxy-3-methyl-butyl)-5-oxo pyrrolidine-1,2-dicarboxylate

To a solution of O1-tert-butyl O2-methyl (2S)-5-oxopyrrolidine-1,2-dicarboxylate (103.39 g, 425.03 mmol, 1 eq) in THE (700 mL) was added LiHMDS (1 M, 552.54 mL, 1.3 eq) at −60° C., the solution was stirred at −60° C. for 0.5 h. Then benzyl N-(1,1-dimethyl-3-oxo-propyl)carbamate (100 g, 425.03 mmol, 1 eq) in THE (700 mL) was added drop-wise at −60° C. The reaction mixture was stirred at −60° C. for 2 h under N₂ atmosphere. Upon completion, the reaction mixture was quenched by addition AcOH (300 mL) in THE (700 mL) at −60° C. and then diluted with H₂O (1000 mL) and extracted with EA (1000 mL 2). The combined organic layers were washed with BRINE (1000 mL), dried over Na₂SO₄, filtered and concentrated under reduced pressure to give a residue. The residue was purified by column chromatography (SiO₂, Petroleum ether:Ethyl acetate=10:1 to 1:1) to give 01-tert-butyl 02-methyl (2S)-4-[3-(benzyloxycarbonylamino)-1-hydroxy-3-methyl-butyl]-5-oxo-pyrrolidine-1,2-dicarboxylate (200 g, crude) as a yellow oil.

Step 4: 01-tert-butyl 02-methyl (2S,4E)-4-[3-(benzyloxycarbonylamino)-3-methyl-butylidene]-5-oxo pyrrolidine-1,2-dicarboxylate

To a solution of O1-tert-butyl O2-methyl (2S)-4-[3-(benzyloxycarbonylamino)-1-hydroxy-3-methyl-butyl]-5-oxo-pyrrolidine-1,2-dicarboxylate (190 g, 397.05 mmol, 1 eq) in DCM (2000 mL) was added with burgess reagent (189.24 g, 794.09 mmol, 2 eq). The reaction mixture was stirred at 40° C. for 2 h. Upon completion, the reaction mixture was diluted with H₂O (1000 mL) and extracted with DCM (1000 mL*2). The combined organic layers were washed with BRINE (500 mL), dried over Na₂SO₄, filtered and concentrated under reduced pressure to give a residue. The residue was purified by column chromatography (SiO₂, Petroleum ether:Ethyl acetate=20:1 to 2:1) to give O1-tert-butyl 02-methyl (2S,4E)-4-[3-(benzyloxycarbonylamino)-3-methyl-butylidene]-5-oxo-pyrrolidine-1,2-dicarboxylate (100 g, 217.15 mmol, 54.69% yield) as a white oil.

Step 5: 01-tert-butyl 02-methyl (2S)-4-(3-amino-3-methyl-butyl)-5-oxo pyrrolidine-1,2-dicarboxylate

To a solution of O1-tert-butyl O2-methyl (2S,4E)-4-[3-(benzyloxycarbonylamino)-3-methyl-butylidene]-5-oxo-pyrrolidine-1,2-dicarboxylate (80 g, 173.72 mmol, 1 eq) in IPA (1000 mL) was added Pd/C (40 g, 173.72 mmol, 10% purity, 1.00 eq). The reaction mixture was stirred at 25° C. for 3 h under H₂ (350.91 mg, 173.72 mmol, 1 eq) at 15 Psi. Upon completion, the reaction mixture was filtered and concentrated under reduced pressure to give O1-tert-butyl 02-methyl (2S)-4-(3-amino-3-methyl-butyl)-5-oxo-pyrrolidine-1,2-dicarboxylate (55 g, crude) as a yellow oil.

Step 6: methyl (2S)-2-(tert-butoxycarbonylamino)-3-(6,6-dimethyl-2-oxo-3-piperidyl)propanoate

To a solution of O1-tert-butyl O2-methyl (2S)-4-(3-amino-3-methyl-butyl)-5-oxo-pyrrolidine-1,2-dicarboxylate (55 g, 167.48 mmol, 1 eq) in MeOH (500 mL) and CHCl₃ (50 mL) was added KOAc (52.27 g, 532.58 mmol, 3.18 eq). The reaction mixture was stirred at 60° C. for 3 h. Upon completion, the reaction mixture was diluted with water (1000 mL) and extracted with DCM (500 mL*3). The combined organic layers were dried over Na₂SO₄, filtered and concentrated under reduced pressure to give a residue. The residue was purified by column chromatography (SiO₂, Petroleum ether:Ethyl acetate=20:1 to 2:1) to give methyl (2S)-2-(tert-butoxycarbonylamino)-3-(6,6-dimethyl-2-oxo-3-piperidyl)propanoate (40 g, 115.71 mmol, 69.09% yield, 95% purity) as a colorless oil.

Step 7: methyl (2S)-2-amino-3-(6,6-dimethyl-2-oxo-3 piperidyl)propanoate

The methyl (2S)-2-(tert-butoxycarbonylamino)-3-(6,6-dimethyl-2-oxo-3-piperidyl)propanoate (1.10 g, 3.35 mmol, 1 eq) in HCUdioxane (4 M, 13 mL, 15.52 eq) was stirred at 25° C. for 1 h. Upon completion, the reaction mixture was concentrated under pressure reduced to get the crude product methyl (2S)-2-amino-3-(6,6-dimethyl-2-oxo-3-piperidyl)propanoate (0.88 g, crude) as white solid. MS (ESI) m/z 209.1 [M+H]⁺

Step 8: methyl (2S)-2-[[(1R,2S,5S)-3-[(2S)-2-(tert-butoxycarbonylamino)-3,3-dimethyl-butanoyl]-6,6-dimethyl-3-azabicyclo[3.1.0]hexane-2-carbonyl]amino]-3-(6,6-dimethyl-2-oxo-3 piperidyl)propanoate

To a solution of methyl (2S)-2-amino-3-(6,6-dimethyl-2-oxo-3-piperidyl)propanoate (880.00 mg, 3.32 mmol, 1 eq, HCl) in DCM (15 mL) was added (1R,2S,5S)-3-[(2S)-2-(tert-butoxycarbonylamino)-3,3-dimethyl-butanoyl]-6,6-dimethyl-3-azabicyclo[3.1.0]hexane-2-carboxylic acid (1.47 g, 3.99 mmol, 1.2 eq) and DMAP (1.22 g, 9.97 mmol, 3 eq) and EDCI (1.27 g, 6.65 mmol, 2 eq). The mixture was stirred at 0° C. for 1 h. Upon completion, the reaction was quenched by addition H₂O (40 mL) and extracted with DCM (10 mL*6). The combined organic layers were dried over Na₂SO₄, filtered and concentrated under reduced pressure and was purified by column chromatography (SiO₂, Petroleum ether:Ethyl acetate=2:1 to 1:1 to 0:1) to give the product methyl (25)-2 [[(1R,2S,5S)-3-[(2S)-2-(tert-butoxycarbonylamino)-3,3-dimethyl-butanoyl]-6,6-dimethyl-3-azabicyclo[3.1.0]hexane-2-carbonyl]amino]-3-(6,6-dimethyl-2-oxo-3-piperidyl)propanoate (2.4 g, 3.07 mmol, 92.32% yield, 74% purity) as yellow solid. MS (ESI) m/z 579.4 [M+H]⁺

Step 9: tert-butyl N-[(1S)-1-[(1R,2S,5S)-2-[[(1S)-2-amino-1-[(6,6-dimethyl-2-oxo-3-piperidyl)methyl]-2-oxo-ethyl]carbamoyl]-6,6-dimethyl-3-azabicyclo[3.1.0]hexane-3-carbonyl]-2,2-dimethyl propyl]carbamate

Methyl (2S)-2-[[(1R,2S,5S)-3-[(2S)-2-(tert-butoxycarbonylamino)-3,3-dimethyl-butanoyl]-6,6-dimethyl-3-azabicyclo [3.1.0]hexane-2-carbonyl]amino]-3-(6,6-dimethyl-2-oxo-3-piperidyl)propanoate (2.40 g, 4.15 mmol, 1 eq) in NH₃/MeOH (7 M, 45 mL, 75.96 eq) was stirred at 25° C. for 16 h. Upon completion, the reaction mixture was concentrated under reduced pressure to give the crude product tert-butyl N-[(1S)-1-[(1R,2S,5S)-2-[[(1S)-2-amino-1-[(6,6-dimethyl-2-oxo-3-piperidyl)methyl]-2-oxo-ethyl]carbamoyl]-6,6-dimethyl-3-azabicyclo[3.1.0]hexane-3-carbonyl]-2,2-dimethyl-propyl]carbamate (2.1 g, crude) as yellow solid. MS (ESI) m/z 564.4 [M+H]⁺

Step 10: (1R,2S,5S)-3-[(2S)-2-amino-3,3-dimethyl-butanoyl]-N-[(1S)-2-amino-1-[(6,6-dimethyl-2-oxo-3 piperidyl)methyl]-2-oxo-ethyl]-6,6-dimethyl-3-azabicyclo[3.1.0]hexane-2-carboxamide

Tert-butyl N-[(1S)-1-[(1R,2S,5S)-2-[[(1S)-2-amino-1-[(6,6-dimethyl-2-oxo-3-piperidyl)methyl]-2-oxo-ethyl]carbamoyl]-6,6-dimethyl-3-azabicyclo[3.1.0]hexane-3-carbonyl]-2,2-dimethyl-propyl]carbamate (500.00 mg, 886.95 umol, 1 eq) in HCl/dioxane (4 M, 8 mL, 36.08 eq) was stirred at 25° C. for 1 h. Upon completion, the reaction was concentrated under reduced pressure to give (1R,2S,5S)-3-[(2S)-2-amino-3,3-dimethyl-butanoyl]-N-[(1S)-2-amino-1-[(6,6-dimethyl-2-oxo-3-piperidyl)methyl]-2-oxo-ethyl]-6,6-dimethyl-3-azabicyclo[3.1.0]hexane-2-carboxamide (440 mg, crude) as yellow solid. MS (ESI) m/z 464.3 [M+H]⁺

Step 11: (1R,2S,5S)—N-[(1S)-2-amino-1-[(6,6-dimethyl-2-oxo-3 piperidyl)methyl]-2-oxo-ethyl]-3-[(2S)-3,3-dimethyl-2-[(2,2,2-trifluoroacetyl)amino]butanoyl]-6,6-dimethyl-3-azabicyclo[3.1.0]hexane-2-carboxamide

To a solution of (1R,2S,5S)-3-[(2S)-2-amino-3,3-dimethyl-butanoyl]-N-[(1S)-2-amino-1-[(6,6-dimethyl-2-oxo-3-piperidyl)methyl]-2-oxo-ethyl]-6,6-dimethyl-3-azabicyclo[3.1.0]hexane-2-carboxamide (440.00 mg, 879.87 umol, 1 eq, HCl) in DCM (10 mL) was added DIPEA (341.14 mg, 2.64 mmol, 459.76 uL, 3 eq) and (2,2,2-trifluoroacetyl) 2,2,2-trifluoroacetate (277.20 mg, 1.32 mmol, 183.58 uL, 1.5 eq). The mixture was stirred at 0° C. for 1 h. Upon completion, the reaction mixture was quenched by water (25 mL). The resulting mixture was extracted with DCM (10 mL*4). The combined organic layers were dried over Na₂SO₄, filtered and concentrated in a vacuum to give the crude product (1R,2S,5S)—N-[(1S)-2-amino-1-[(6,6-dimethyl-2-oxo-3-piperidyl)methyl]-2-oxo-ethyl]-3-[(2S)-3,3-dimethyl-2-[(2,2,2-trifluoroacetyl)amino]butanoyl]-6,6-dimethyl-3-azabicyclo[3.1.0]hexane-2-carboxamide (440 mg, crude) as yellow solid. MS (ESI) m/z 560.3 [M+H]⁺

Step 12: (1R,2S,5S)—N-[(1S)-1-cyano-2-(6,6-dimethyl-2-oxo-3 piperidyl)ethyl]-3-[(2S)-3,3-dimethyl-2-[(2,2,2-trifluoroacetyl)amino]butanoyl]-6,6-dimethyl-3-azabicyclo[3.1.0]hexane-2-carboxamide

To a solution of (1R,2S,5S)—N-[(1S)-2-amino-1-[(6,6-dimethyl-2-oxo-3-piperidyl)methyl]-2-oxo-ethyl]-3-[(2S)-3,3-dimethyl-2-[(2,2,2-trifluoroacetyl)amino]butanoyl]-6,6-dimethyl-3-azabicyclo[3.1.0]hexane-2-carboxamide (420.00 mg, 750.51 umol, 1 eq) in DCM (8 mL) was added burgess reagent (536.56 mg, 2.25 mmol, 3 eq). The mixture was stirred at 25° C. for 2 h. Upon completion, the reaction mixture was quenched with water (0.2 mL) and blow-dried with N₂ and was purified by prep-HPLC (column: Waters Xbridge Prep OBD C18 150*40 mm*10 um; mobile phase: [water (NH₄HCO₃)-ACN]; B %: 35%-65%, 8 min) and re-purified by SRC (column: DAICEL CHIRALCEL OJ(250 mm*30 mm, 10 um); mobile phase: [0.1% NH₃H₂O ETOH]; B %: 13% 13%, 15 min) to give the product (1R,5S)—N-[(1S)-1-cyano-2-(6,6-dimethyl-2-oxo-3-piperidyl)ethyl]-3-[(2S)-3,3-dimethyl-2-[(2,2,2-trifluoroacetyl)amino]butanoyl]-6,6-dimethyl-3-azabicyclo[3.1.0]hexane-2-carboxamide (51 mg, 94.16 umol, 51.00% yield) as white solid. MS (ESI) m/z 542.3 [M+H]⁺

¹H NMR (400 MHz, DMSO-d₆) δ=9.41 (br s, 1H), 9.00 (d, J=8.3 Hz, 1H), 7.47 (s, 1H), 5.13-4.90 (m, 1H), 4.41 (br s, 1H), 4.16 (s, 1H), 3.90 (br dd, J=5.4, 10.3 Hz, 1H), 3.68 (br d, J=10.5 Hz, 1H), 2.39-2.26 (m, 1H), 2.14 (br d, J=5.5 Hz, 1H), 1.83-1.70 (m, 2H), 1.62-1.44 (m, 4H), 1.29 (d, J=7.5 Hz, 1H), 1.12 (br d, J=5.4 Hz, 6H), 1.05-1.01 (m, 3H), 0.98 (s, 9H), 0.89-0.82 (m, 3H)

¹H NMR (400 MHz, DMSO-d₆) δ=9.03-8.84 (m, 1H), 8.74 (d, J=7.7 Hz, 1H), 7.27-7.07 (m, 1H), 4.99 (ddd, J=6.4, 7.9, 9.5 Hz, 1H),4.52-4.37 (m, 1H), 4.30-4.13 (m, 1H), 3.99-3.87 (m, 1H), 3.71 (d, J=10.3 Hz, 1H), 2.32 (ddd, J=6.0, 9.8, 13.6 Hz, 1H), 2.25-2.15 (m, 1H), 1.89-1.75 (m, 2H), 1.66-1.51 (m, 4H), 1.31 (d, J=7.6 Hz, 1H), 1.17 (d, J=6.4 Hz, 6H), 1.07-1.03 (m, 3H), 1.01 (s, 9H), 0.85 (s, 3H).

Example 92: Synthesis of (1R,2S,5S)—N-[(1S)-1-cyano-2-[(7R)-6-oxo-5-azaspiro[3.4]octan-7-yl]ethyl]-3-[(2S)-3,3-dimethyl-2-[(2,2,2-trifluoroacetyl)amino]butanoyl]-6,6-dimethyl-3-azabicyclo[3.1.0]hexane-2-carboxamide (Compound 362)

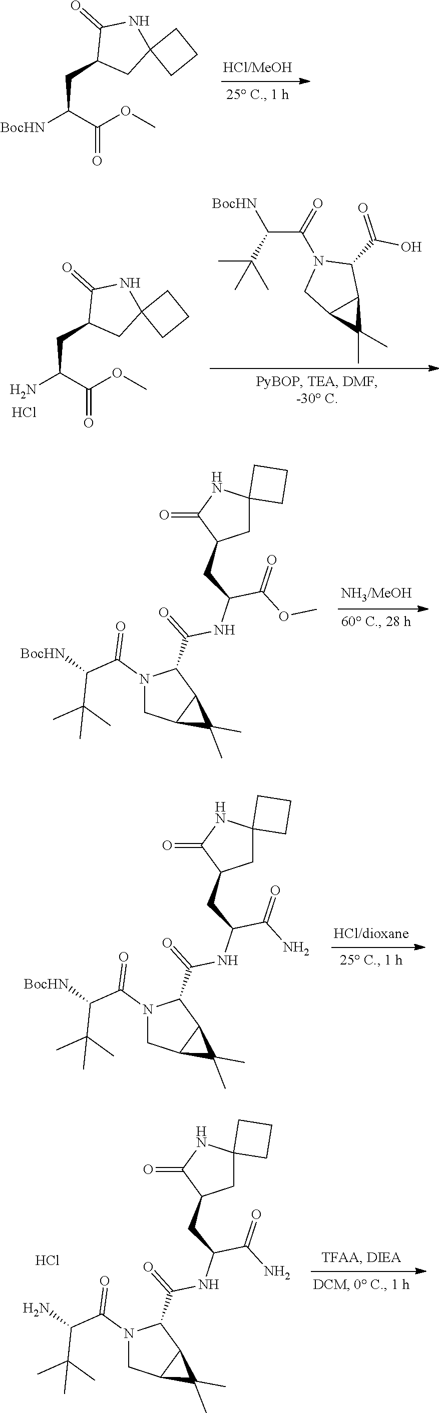

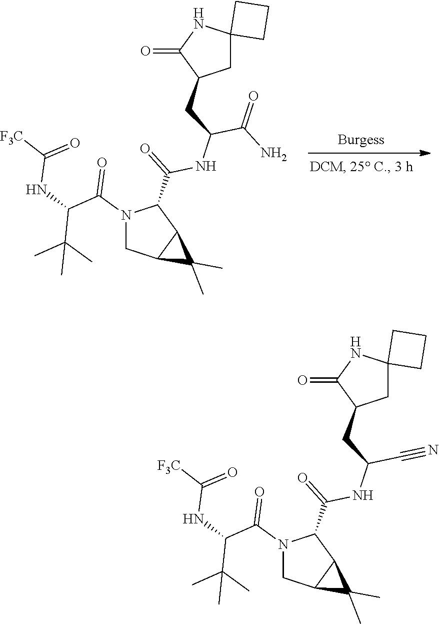 [SMILES: CC(C)(C)[C@H](NC(=O)C(F)(F)F)C(=O)N1C[C@H]2[C@@H]([C@H]1C(=O)N[C@@H](C[C@@H]1CC3(CCC3)NC1=O)C(N)=O)C2(C)C ; CC(C)(C)[C@H](NC(=O)C(F)(F)F)C(=O)N1C[C@H]2[C@@H]([C@H]1C(=O)N[C@H](C#N)C[C@@H]1CC3(CCC3)NC1=O)C2(C)C]

Step 1: methyl (2S)-2-amino-3-[(7R)-6-oxo-5-azaspiro[3.4]octan-7-yl]propanoate

A solution of methyl (2S)-2-(tert-butoxycarbonylamino)-3-[(7R)-6-oxo-5-azaspiro[3.4]octan-7-yl]propanoate (400 mg, 1.23 mmol, 1 eq) in HCl/MeOH (4 M, 4 mL, 13.06 eq) was stirred at 25° C. for 1 h. Upon completion, the reaction mixture was concentrated under reduced pressure to dryness to give methyl (2S)-2-amino-3-[(7R)-6-oxo-5-azaspiro[3.4]octan-7-yl]propanoate (370 mg, crude, HCl) as white solid. MS (ESI) m/z 227.1 [M+H]⁺.

Step 2: methyl (2S)-2-[[(1R,2S,5S)-3-[(2S)-2-(tert-butoxycarbonylamino)-3,3-dimethyl-butanoyl]-6,6-dimethyl-3-azabicyclo[3.1.0]hexane-2-carbonyl]amino]-3-[(7R)-6-oxo-5-azaspiro[3.4]octan-7-yl]propanoate

To a mixture of methyl (2S)-2-amino-3-[(7R)-6-oxo-5-azaspiro[3.4]octan-7-yl]propanoate (330 mg, 1.09 mmol, 87% purity, 1 eq, HCl) and (1R,2S,5S)-3-[(2S)-2-(tert-butoxycarbonylamino)-3,3-dimethyl-butanoyl]-6,6-dimethyl-3-azabicyclo[3.1.0]hexane-2-carboxylic acid (398.57 mg, 1.07 mmol, 99% purity, 0.98 eq) in DMF (3 mL) was added (benzotriazol-1-yloxytripyrrolidinophosphonium hexafluorophosphate) (682.39 mg, 1.31 mmol, 1.2 eq), then a solution of TEA (331.73 mg, 3.28 mmol, 456.29 uL, 3 eq) in DMF (1 mL) was added and stirred at −30° C. for 2 h. Upon completion, the reaction mixture was quenched by water (20 mL), and then extracted with DCM (10 mL*2). The combined organic layers were washed with brine (10 mL), dried over Na₂SO₄, filtered and concentrated under reduced pressure to give a residue. The residue was purified by column (SiO₂, PE:EA=10:1 to 0:1) to give methyl (2S)-2-[[(1R,2S,5S)-3-[(2S)-2-(tert-butoxycarbonylamino)-3,3-dimethyl-butanoyl]-6,6-dimethyl-3-azabicyclo[3.1.0]hexane-2-carbonyl]amino]-3-[(7R)-6-oxo-5-azaspiro[3.4]octan-7-yl]propanoate (490 mg, 722.18 umol, 66.09% yield, 85% purity) as yellow solid. MS (ESI) m/z 577.4 [M+H]⁺.

Step 3: tert-butyl N-[(1S)-1-[(1R,2S,5S)-2-[[(1S)-2-amino-2-oxo-1-[[(7R)-6-oxo-5-azaspiro[3.4]octan-7-yl]methyl]ethyl]carbamoyl]-6,6-dimethyl-3-azabicyclo[3.1.0]hexane-3-carbonyl]-2,2-dimethyl propyl]carbamate

A solution of methyl (2S)-2-[[(1R,2S,5S)-3-[(2S)-2-(tert-butoxycarbonylamino)-3,3-dimethyl-butanoyl]-6,6-dimethyl-3-azabicyclo[3.1.0]hexane-2-carbonyl]amino]-3-[(7R)-6-oxo-5-azaspiro[3.4]octan-7-yl]propanoate (490 mg, 722.18 umol, 85% purity, 1 eq) in NH₃/MeOH (7 M, 10 mL, 96.93 eq) was stirred at 60° C. for 28 h. Upon completion, the reaction mixture was concentrated under reduced pressure to dryness to give tert-butyl N [(1S)-1-[(1R,2S,5S)-2-[[(1S)-2-amino-2-oxo-1-[[(7R)-6-oxo-5-azaspiro[3.4]octan-7-yl]methyl]ethyl]carbamoyl]-6,6-dimethyl-3-azabicyclo[3.1.0]hexane-3-carbonyl]-2,2-dimethyl-propyl]carbamate (470 mg, crude) as yellow solid. MS (ESI) m/z 562.3 [M+H]⁺.

Step 4: (1R,2S,5S)-3-[(2S)-2-amino-3,3-dimethyl-butanoyl]-N-[(1S)-2-amino-2-oxo-1-[[(7R)-6-oxo-5-azaspiro[3.4]octan-7-yl]methyl]ethyl]-6,6-dimethyl-3-azabicyclo[3.1.0]hexane-2-carboxamide

A solution of tert-butyl N-[(1S)-1-[(1R,2S,5S)-2-[[(1S)-2-amino-2-oxo-1-[[(7R)-6-oxo-5-azaspiro[3.4]octan-7-yl]methyl]ethyl]carbamoyl]-6,6-dimethyl-3-azabicyclo[3.1.0]hexane-3-carbonyl]-2,2-dimethyl-propyl]carbamate (470 mg, 711.22 umol, 85% purity, 1 eq) in HCl/dioxane (4 M, 5 mL, 28.12 eq) was stirred at 25° C. for 1 h. Upon completion, the mixture was concentrated under reduced pressure to dryness to give (1R,2S,5S)-3-[(2S)-2-amino-3,3-dimethyl-butanoyl]-N-[(1S)-2-amino-2-oxo-1-[[(7R)-6-oxo-5-azaspiro[3.4]octan-7-yl]methyl]ethyl]-6,6-dimethyl-3-azabicyclo[3.1.0]hexane-2-carboxamide (415 mg, crude, HCl) as white solid. MS (ESI) m/z 462.3 [M+H]⁺.

Step 5: (1R,2S,5S)—N-[(1S)-2-amino-2-oxo-1-[[(7R)-6-oxo-5-azaspiro[3.4]octan-7-yl]methyl]ethyl]-3-[(2S)-3,3-dimethyl-2-[(2,2,2-trifluoroacetyl)amino]butanoyl]-6,6-dimethyl-3-azabicyclo[3.1.0]hexane-2-carboxamide

A mixture of (1R,2S,5S)-3-[(2S)-2-amino-3,3-dimethyl-butanoyl]-N-[(1S)-2-amino-2-oxo-1-[[(7R)-6-oxo-5-azaspiro[3.4]octan-7-yl]methyl]ethyl]-6,6-dimethyl-3-azabicyclo[3.1.0]hexane-2-carboxamide (400 mg, 682.65 umol, 85% purity, 1 eq, HCl) and DIEA (264.68 mg, 2.05 mmol, 356.71 uL, 3 eq) in DCM (10 mL) was added a solution of TFAA (215.07 mg, 1.02 mmol, 142.43 uL, 1.5 eq) in DCM (2 mL) slowly at 0° C., then the mixture was stirred at 0° C. for 1 h. Upon completion, the reaction mixture was diluted with water (10 mL) and extracted with DCM (10 mL*3), the combined organic layers were dried over Na₂SO₄, filtered and concentrated under reduced pressure to give (1R,2S,5S)—N-[(1S)-2-amino-2-oxo-1-[[(7R)-6-oxo-5-azaspiro[3.4]octan-7-yl]methyl]ethyl]-3-[(2S)-3,3-dimethyl-2-[(2,2,2-trifluoroacetyl)amino]butanoyl]-6,6-dimethyl-3-azabicyclo[3.1.0]hexane-2-carboxamide (460 mg, crude) as yellow solid. MS (ESI) m/z 558.3 [M+H]⁺.

Step 6: (1R,2S,5S)—N-[(1S)-1-cyano-2-[(7R)-6-oxo-5-azaspiro[3.4]octan-7-yl]ethyl]-3-[(2S)-3,3-dimethyl-2-[(2,2,2-trifluoroacetyl)amino]butanoyl]-6,6-dimethyl-3-azabicyclo[3.1.0]hexane-2-carboxamide

A mixture of (1R,2S,5S)—N-[(1S)-2-amino-2-oxo-1-[[(7R)-6-oxo-5-azaspiro[3.4]octan-7-yl]methyl]ethyl]-3-[(25)-3,3-dimethyl-2-[(2,2,2-trifluoroacetyl)amino]butanoyl]-6,6-dimethyl-3-azabicyclo[3.1.0]hexane-2-carboxamide (460 mg, 643.47 umol, 78% purity, 1 eq) in DCM (10 mL) was added burgess reagent (460.03 mg, 1.93 mmol, 3 eq) and stirred at 25° C. for 3 h. Upon completion, the mixture was quenched with water (0.5 mL) and concentrated under reduced pressure (<30° C.) to give a residue. The residue was purified by prep-HPLC (column: Waters Xbridge BEH C18 250*50 mm*10 um; mobile phase: [water (NH₄HCO₃)-ACN]; B %: 40%-60%, 10 min) to give (1R,2S,5S)—N-[(1S)-1-cyano-2-[(7R)-6-oxo-5-azaspiro[3.4]octan-7-yl]ethyl]-3-[(2S)-3,3-dimethyl-2-[(2,2,2-trifluoroacetyl)amino]butanoyl]-6,6-dimethyl-3-azabicyclo[3.1.0]hexane-2-carboxamide (193.78 mg, 359.13 umol, 55.81% yield, 100% purity) as white solid. MS (ESI) m/z 540.2 [M+H]⁺.

¹H NMR (400 MHz, DMSO-d₆) δ=9.50-9.23 (m, 1H), 8.99 (d, J=8.4 Hz, 1H), 8.17 (s, 1H), 4.94 (ddd, J=5.8, 8.4, 10.3 Hz, 1H), 4.40 (s, 1H), 4.17 (s, 1H), 3.90 (dd, J=5.5, 10.4 Hz, 1H), 3.69 (d, J=10.5 Hz, 1H), 2.46-2.37 (m, 1H), 2.35-2.20 (m, 2H), 2.18-2.08 (m, 1H), 2.07-1.88 (m, 3H), 1.80-1.68 (m, 2H), 1.64-1.50 (m, 3H), 1.29 (d, J=7.5 Hz, 1H), 1.10-1.02 (m, 3H), 0.99 (s, 9H), 0.89-0.84 (m, 3H).

¹H NMR (400 MHz, DMSO-d₆, 273+80K) δ=9.06-8.85 (m, 1H), 8.77 (br d, J=7.9 Hz, 1H), 7.95 (s, 1H), 4.94 (ddd, J=6.1, 8.1, 9.7 Hz, 1H), 4.45 (s, 1H), 4.23 (s, 1H), 3.92 (dd, J=5.5, 10.4 Hz, 1H), 3.71 (d, J=10.4 Hz, 1H), 2.47-2.41 (m, 1H), 2.40-2.23 (m, 2H), 2.21-2.11 (m, 1H), 2.10-2.03 (m, 2H), 1.97 (tdd, J=3.9, 7.7, 11.4 Hz, 1H), 1.83 1.71 (m, 2H), 1.69-1.53 (m, 3H), 1.31 (d, J=7.6 Hz, 1H), 1.08-0.96 (m, 12H), 0.93-0.85 (m, 3H).

Example 93: Synthesis of (1R,2S,5S)—N-[(1S)-1-cyan-2-[(6R)-5-oxo-4-azaspiro[2.4]heptan-6-yl]ethyl]-3-[2-cyclobutyl-2-[(2,2,2-trifluoroacetyl)amino]acetyl]-6,6-dimethyl-3-azabicyclo[3.1.0]hexane-2-carboxamide (Compound 372)

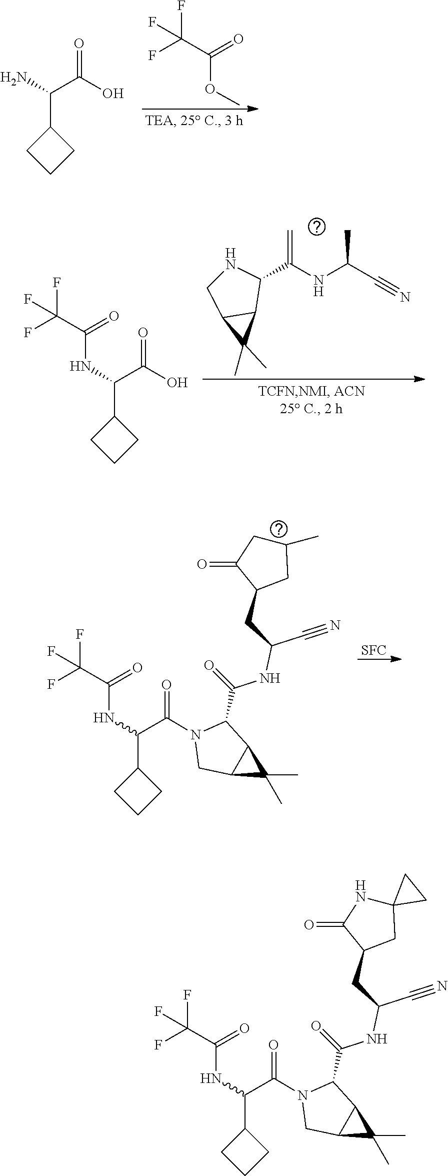

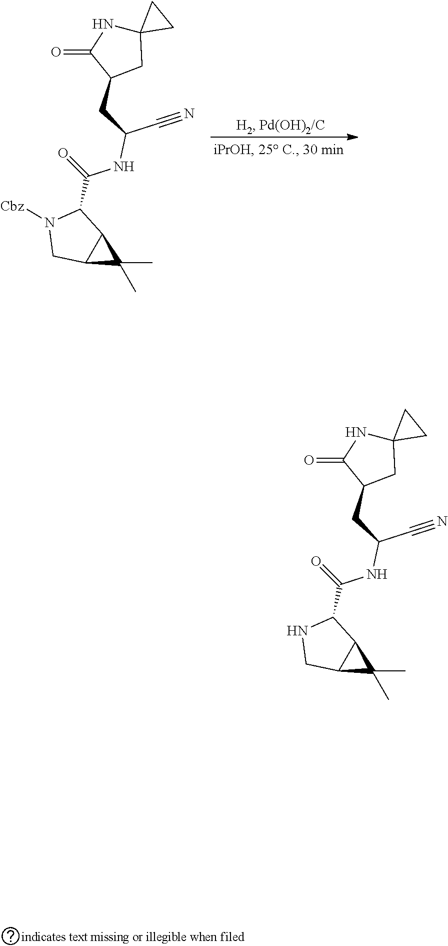 [SMILES: CC1(C)[C@@H]2[C@@H](C(=O)N[C@H](C#N)C[C@@H]3CC4(CC4)NC3=O)N(C(=O)OCc3ccccc3)C[C@@H]21 ; "H2, Pd(OH)2/C" ; CC1(C)[C@@H]2[C@@H](C(=O)N[C@H](C#N)C[C@@H]3CC4(CC4)NC3=O)NC[C@@H]21]

Step 1: (1R,2S,5S)—N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide

To a solution of benzyl (1R,2S,5S)-2-[[(1S)-1-cyano-2-[(6R)-5-oxo-4-azaspiro[2.4]heptan-6-yl]ethyl]carbamoyl]-6,6-dimethyl-3-azabicyclo[3.1.0]hexane-3-carboxylate (500 mg, 1.11 mmol, 1 eq) in i-PrOH (5 mL) was added Pd(OH)₂/C (150 mg, 20% purity) under N₂. The suspension was degassed under vacuum and purged with H₂ several times. The mixture was stirred under H₂ (2.24 mg, 1.11 mmol, 1 eq) (15 psi) at 25° C. for 30 min. Upon completion, the reaction mixture was filtered and the filtrate was concentrated to give (1R,2S,5S)—N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (320 mg, crude) as a white solid.

Step 2: 2-cyclobutyl-2-[(2,2,2-trifluoroacetyl)amino]acetic acid

To a solution of (2S)-2-amino-2-cyclobutyl-acetic acid (350 mg, 2.71 mmol, 1 eq) in methyl 2,2,2-trifluoroacetate (4.45 g, 34.71 mmol, 3.50 mL, 12.81 eq) was added TEA (822.64 mg, 8.13 mmol, 1.13 mL, 3 eq). The mixture was stirred at 25° C. for 3 h. Upon completion, the reaction mixture was concentrated under reduced pressure. The residue was diluted with H₂O 30 mL. The aqueous phase was adjusted pH to around 2 with 0.5 M HCl at 0° C. and then extracted with DCM (15 mL*3). The combined organic phase was washed with brine (20 mL), dried over anhydrous Na₂SO₄, filtered and concentrated in vacuo. To give a 2-cyclobutyl-2-[(2,2,2-trifluoroacetyl)amino]acetic acid (400 mg, crude) as white solid.

Step 3: (1R,2S,5S)—N-[(1S)-1-cyano-2-[(6R)-5-oxo-4-azaspiro[2.4]heptan-6-yl]ethyl]-3-[(2S)-2-cyclobutyl-2-[(2,2,2-trifluoroacetyl)amino]acetyl]-6,6-dimethyl-3-azabicyclo[3.1.0]hexane-2-carboxamide

To a solution of (2S)-2-cyclobutyl-2-[(2,2,2-trifluoroacetyl)amino]acetic acid (273.27 mg, 1.21 mmol, 1.2 eq) and (1R,2S,5S)—N-[(1S)-1-cyano-2-[(6R)-5-oxo-4-azaspiro[2.4]heptan-6-yl]ethyl]-6,6-dimethyl-3-azabicyclo[3.1.0]hexane-2-carboxamide (320 mg, 1.01 mmol, 1 eq) in ACN (5 mL) was added TCFH (425.66 mg, 1.52 mmol, 1.5 eq) and NMI (166.08 mg, 2.02 mmol, 161.24 uL, 2 eq). Upon completion, the mixture was stirred at 25° C. for 2 h. Upon completion, the residue was poured into H₂O (50 mL). The aqueous phase was extracted with ethyl acetate (30 mL*3). The combined organic phase was washed with brine (50 mL), dried with anhydrous Na₂SO₄, filtered and concentrated in vacuum. The residue was purified by prep-HPLC (neutral condition; column: Waters Xbridge BEH C18 250*50 mm*10 um; mobile phase: [water(NH₄HCO₃)-ACN]; B %: 30%-50%, 10 min) to give (1R,2S,5S)—N-[(1S)-1-cyano-2-[(6R)-5-oxo-4-azaspiro[2.4]heptan-6-yl]ethyl]-3-[(2S)-2-cyclobutyl-2-[(2,2,2-trifluoroacetyl)amino]acetyl]-6,6-dimethyl-3-azabicyclo[3.1.0]hexane-2-carboxamide (300 mg, 573.01 umol, 56.66% yield) as white solid. MS (ESI) m/z 524.3 [M+H]⁺.

Step 4: (1R,2S,5S)—N-[(1S)-1-cyano-2-[(6R)-5-oxo-4-azaspiro[2.4]heptan-6-yl]ethyl]-3-[(2S)-2-cyclobutyl-2-[(2,2,2-trifluoroacetyl)amino]acetyl]-6,6-dimethyl-3-azabicyclo[3.1.0]hexane-2-carboxamide

(1R,2S,5S)—N-[(1S)-1-cyano-2-[(6R)-5-oxo-4-azaspiro[2.4]heptan-6-yl]ethyl]-3-[2-cyclobutyl-2-[(2,2,2-trifluoroacetyl)amino]acetyl]-6,6-dimethyl-3-azabicyclo[3.1.0]hexane-2-carboxamide (0.3 g, 573.01 umol, 1 eq) was further separated by SFC (condition:column: DAICEL CHIRALPAK IC(250 mm*30 mm, 10 um); mobile phase: [Neu-PA]; B %: 40%-40%, 10 min) to give (1R,2S,5S)—N-[(1S)-1-cyano-2-[(6R)-5-oxo-4-azaspiro[2.4]heptan-6-yl]ethyl]-3-[2-cyclobutyl-2-[(2,2,2-trifluoroacetyl)amino]acetyl]-6,6-dimethyl-3-azabicyclo[3.1.0]hexane-2-carboxamide Isomer 1 (95 mg, 181.45 umol, 31.67% yield, 100% purity) as a white solid. MS (ESI) m/z 524.3 [M+H]⁺.

¹H NMR (400 MHz, DMSO-d₆) δ=9.71 (brd, J=5.7 Hz, 1H), 8.94 (d, J=8.3 Hz, 1H), 7.77 (s, 1H), 4.98-4.87 (m, 1H), 4.45 (br d, J=10.1 Hz, 1H), 4.13 (s, 1H), 3.91 (dd, J=5.4, 10.2 Hz, 1H), 3.75 (d, J=10.4 Hz, 1H), 2.74-2.58 (m, 2H), 2.17 (ddd, J=4.8, 10.4, 13.5 Hz, 1H), 2.03-1.90 (m, 3H), 1.88-1.72 (m, 6H), 1.57 (dd, J=5.5, 7.3 Hz, 1H), 1.29 (d, J=7.6 Hz, 1H), 1.05-1.00 (m, 3H), 0.88 (s, 3H), 0.80-0.71 (m, 1H), 0.65-0.52 (m, 3H)

(1R,2S,5S)—N-[(1S)-1-cyano-2-[(6R)-5-oxo-4-azaspiro[2.4]heptan-6-yl]ethyl]-3-[2-cyclobutyl-2-[(2,2,2-trifluoroacetyl)amino]acetyl]-6,6-dimethyl-3-azabicyclo[3.1.0]hexane-2-carboxamide Isomer 2 (66 mg, 117.24 umol, 20.46% yield, 93% purity) was obtained as a white solid. MS (ESI) m/z 524.3 [M+H]⁺.

¹H NMR (400 MHz, DMSO-d₆) δ=9.68 (brd, J=7.9 Hz, 1H), 9.45-8.62 (m, 1H), 7.85-7.72 (m, 1H), 5.07-4.86 (m, 1H), 4.60-4.05 (m, 2H), 3.92-3.45 (m, 2H), 2.78-2.60 (m, 2H), 2.29-2.09 (m, 1H), 2.03-1.67 (m, 8H), 1.61-1.54 (m, 1H), 1.48-1.37 (m, 1H), 1.29 (d, J=7.6 Hz, 1H), 1.07-0.98 (m, 3H), 0.94-0.78 (m, 3H), 0.77-0.71 (m, 1H), 0.62-0.51 (m, 3H)

¹H NMR (400 MHz, ACETONITRILE-d3) δ=8.23-7.92 (m, 1H), 7.86-7.63 (m, 1H), 6.33-6.09 (m, 1H), 4.95-4.77 (m, 1H), 4.61-4.46 (m, 1H), 4.13 (s, 1H), 4.00 (dd, J=5.4, 10.5 Hz, 1H), 3.74-3.56 (m, 1H), 2.82-2.65 (m, 2H), 2.17-2.05 (m, 5H), 2.03 1.97 (m, 2H), 1.88-1.77 (m, 3H), 1.56-1.42 (m, 2H), 1.07-0.99 (m, 3H), 0.94 (s, 3H), 0.85-0.80 (m, 1H), 0.72-0.57 (m, 3H)

Example 94: Evaluation of Broad-Spectrum Coronaviral 3CL Pro Inhibitors

CoV 3CL's representing members from each of the α, β, γ phylogenetic groups and subgroups can be expressed and purified to high purity to be assayed for inhibition by the designed library: FIPV-, PEDV-, and NL63-3CL m from the a-CoV lineage, HKU1-, OC43-, SARS-, HKU4-, HKU5-, and HKU9-3CLpro from the β-CoV lineage, and IBV-3CLpro from the yCoV lineage (St. John, et al., Bioorg Med Chem Lett 2015, 25(22):5072 5077; Grum-Tokars, et al., Virus Res 2008, 133(1):63-73). The compounds can tested against all ten 3CLpro's individually to determine inhibition. For example, the enzymatic activity of a given 3CLpro in the presence of a library member at a concentration of 100 μM is measured using a synthetic FRET peptide substrate containing the consensus nsp4-nsp5 cleavage site known for 3CLpro's: HilyteFluor.TM.-488-ESATLQSGLRKAK-(QXL 520)-NH₂ (AnaSpec, Inc.). ICso values are then determined for compounds that produced greater than 50% inhibition of a given 3CLpro at 100 W.

Example 95: Evaluation of Antiviral Activity of Compounds Against COVID-19 (nCoV-2019, SARS-CoV2) Mpro in the Enzymatic Assay

Compounds were assayed using standard methods to assess compound activity and IC50. As an exemplary for assessment of the SARS-COV2 Mpro, the C-His6-tagged Mpro (NC_045512) was cloned, expressed in E. coli and purified. The assay buffer contained 20 mM of Tris-HCl (pH 7.3), 100 mM of NaCl, 1 mM of EDTA, 5 mM of TCEP and 0.1% BSA. The final concentrations of the Mpro protein and substrate were 25 nM and 25 μM, respectively, in the Mpro enzymatic assay. The Km of the Mpro substrate for the protease was 13.5 μM.

The compounds were added to an assay plate. For 100% inhibition control (HPE, hundred percent effect), 1 μM GC376 was added. For no inhibition control (ZPE, zero percent effect), no compound was added. Each activity testing point had a relevant background control to normalize the fluorescence interference of compound.

IC50 values of compounds were calculated with the GraphPad Prism software using the nonlinear regression model of log(inhibitor) vs. response—Variable slope (four parameters). The inhibition activity was calculated using the formula below, IC50 values is calculated using the Inhibition % data.

$$Inhibition \% = [ ( Sample - Average ZPE ) / ( Average HPE - Average ZPE ) ] * 100 {\%}^{\#}$$

#HEP: Hundred percent effect controls. Containing substrate+enzyme+1 μM GC376.
ZPE: Zero percent effective controls. Containing enzyme+substrate, no compound.
Sample: Compound activity testing wells. Containing compound+enzyme+substrate.
BG: Compound background control wells. Containing compound+substrate, no enzyme.

Example 96: Evaluation of Antiviral Activity of Compounds Against Human Coronavirus (HCov) 229E and OC43 in the Cytopathic Effect (CPE) Assays

Compounds were assayed using standard methods against multiple coronaviral strains, including HCoV 229E and OC43 strains. The antiviral activity of compounds was calculated based on the protection of the virus-induced CPE at each concentration normalized by the virus control.

Reagents and instruments used in this assay include luminescent cell viability assay kit CellTiter Glo (Promega) and Microplate Reader Synergy2 (BioTek).

### Virus—HCoV 229E

Cytopathic effect (CPE) was measured by CellTiter Glo following the manufacturer's manual. The antiviral activity of compounds was calculated based on the protection of the virus-induced CPE at each concentration normalized by the virus control.

### Virus—HCov OC43

Reference compound used was remdesivir; detection reagent: CellTiter Glo.) The CPE were measured by CellTiter Glo following the manufacturer's manual. The antiviral activity of compounds was calculated based on the protection of the virus-induced CPE at each concentration normalized by the virus control.

The cytotoxicity of compounds was assessed under the same conditions, but without virus infection, in parallel. Cell viability was measured with CellTiter Glo. The antiviral activity and cytotoxicity of compounds were expressed as % Inhibition and % Viability, respectively, and calculated with formulas.

Example 97. Evaluation the In Vitro Antiviral Activity of Compounds Against SARS-CoV-2 in the Cell-Based Replicon Assay

Compounds are assayed at 8 concentrations, 3-fold dilution, in duplicate wells. The highest test concentrations of test compounds and reference compound is 10 μM and 1 μM, respectively. The main reagents used in this assay was luminescent cell viability assay kit CellTiter Glo (Promega). The main instrument used in this assay was Acumen Cellista (TTP LabTech).

The antiviral assay is summarized in Table 2b.

TABLE 2b
Method of the SARS-CoV-2 replicon assay

| | | Compound | | |
|---|---|---|---|---|
| | | treatment | | |
| | | duration | Reference | Detection |
| Replicon | Cell line | (day)/Endpoint | compound | reagent |
| SARS- | Huh7, | 1/GFP | GC376 | CellTiter |
| COV- | 4000 cells | | | Glo. |
| 2WT | per 384-W | | | |

The replicon RNA was generated by the mMACHINE Ti Ultra Kit. Huh7 cells were transfected with purified replicon RNAs and seeded in plates containing serially diluted compounds, then cultured at 37° C. and 5% CO₂ for 1 day. The final volume of the cell culture was 60 μl per well, and the final concentrations of DMSO in the test plates are 0.5%. Fluorescence intensity were determined using Acumen Cellista (TTP LabTech), and the antiviral activity of compounds were calculated based on the inhibition of expression of GFP. Cell viability were measured with CellTiter Glo following the manufacturer's manual.

The antiviral activity and cytotoxicity of compounds were expressed as % Inhibition and % Viability, respectively, and calculated with the formulas below:

$$Inhibition ( \% ) = ( Raw {data}_{CPD} - {Average}_{ZPE} ) / ( {Average}_{HPE} - {Average}_{ZPE} ) * 100$$

$$Viability ( \% ) = ( Raw {data}_{CPD} - {Average}_{HPE} ) / ( {Average}_{ZPE} - {Average}_{HPE} ) * 100$$

Raw data cpv: values of the sample-treated wells
Average zpe: average value of no-compound treatment control
Average HPE: average value of medium control (medium only) wells.

EC₅₀ and CC₅₀ values were calculated using the GraphPad Prism software using the nonlinear regression model of log(inhibitor) vs. response—Variable slope (four parameters).

Table 3, Table 4, Table 5 and Table 6 show activity data. In some embodiments, certain stereoisomers of disclosed compounds may have significant activity as compared to other stereoisomers of the same compound.

TABLE 3
Activity data for compounds.

| Compound | 229E | COVID-19 | OC43 |
|---|---|---|---|
| No. | Mpro IC₅₀ | Mpro IC₅₀ | Mpro IC₅₀ |
| 101 Isomer 1 | D | D | D |
| 101 Isomer 2 | D | D | D |
| 102 | C | A | B |
| 111 | D | C | D |
| 113 | D | D | D |
| 115 | D | C | D |
| 116 | D | D | D |
| 117 | D | D | D |
| 118 | D | C | D |
| 119 | D | C | D |
| 120 | D | C | D |
| 121 | D | C | D |
| 123 | D | B | C |
| 125 | D | C | D |
| 128 | D | D | D |
| 110 | D | D | D |
| 129 | D | D | D |
| 132 | D | D | D |
| 133 | D | D | D |
| 134 | D | D | D |
| 135 | D | D | D |
| 136 | D | D | D |
| 235 | D | D | D |
| 299 Isomer 1 | D | D | D |
| 308 | D | D | D |
| 331 | D | D | D |
| 315 | D | D | D |
| 324 | D | D | D |
| 328 | D | D | D |
| 330 | D | D | D |
| 316 | A | D | B |
| 285 | D | D | D |
| 307 | D | D | D |
| 292 Isomer 1 | D | D | D |
| 292 Isomer 2 | D | D | D |
| 273 Isomer 1 | D | D | D |
| 273 Isomer 2 | D | D | D |
| 361 | B | D | D |
| 362 | D | D | D |
| 372 Isomer 1 | D | D | D |
| 372 Isomer 2 | D | D | D |
| 373 Isomer 1 | D | D | D |
| 373 Isomer 2 | D | D | D |
| 375 | D | D | D |

A ≥ 30 μM, B > 10 μM and <30 μM, C ≥ 2 μM and ≤10 μM, D < 2 μM.

TABLE 4
Activity data for compounds.

| Compound No. | 229E CPE EC₅₀ | OC43 CPE EC₅₀ |
|---|---|---|
| 101 Isomer 1 | C | D |
| 101 Isomer 2 | C | D |
| 111 | B | C |
| 113 | C | C |
| 115 | C | C |
| 116 | C | D |
| 118 | C | C |
| 119 | B | C |
| 120 | C | D |
| 121 | C | C |
| 123 | B | B |
| 128 | D | D |
| 110 | D | |
| 129 | D | |
| 132 | D | |
| 133 | D | |
| 134 | D | |
| 135 | D | |
| 136 | D | |

A ≥ 30 μM, B > 10 μM and ≤30 μM, C ≥ 2 μM and ≤10 μM, D < 2 μM.

TABLE 5
Activity data for compounds.

| Compound | COVID-19 | 229E | OC43 |
|---|---|---|---|
| No. | Mpro IC₅₀ | Mpro IC₅₀ | Mpro IC₅₀ |
| 139 | D | D | D |
| 137 | D | D | D |
| 138 | D | D | D |
| 284 | D | B | D |
| 290 | D | D | D |
| 295 | D | D | D |
| 298 Isomer 1 | D | D | D |
| 298 Isomer 2 | D | D | D |
| 299 Isomer 1 | D | D | D |
| 299 Isomer 2 | D | D | D |
| 309 | D | D | D |
| 314 | D | D | D |
| 312 | D | D | D |
| 327 | D | D | D |
| 319 | D | D | D |
| 326 | D | D | D |
| 425 | D | D | D |
| 357 | D | D | D |
| 334 | D | D | D |
| 358 | D | D | D |
| 430 | D | D | D |
| 431 | D | D | D |
| 335 | D | D | D |

A ≥ 30 μM, B > 10 μM and <30 μM, C ≥ 2 μM and ≤10 μM, D < 2 μM.

TABLE 6
Activity data for compounds.

| Compound | Replicon |
|---|---|
| No. | EC₅₀ (μM) |
| 376 | D |
| 377 | D |
| 378 | D |

A ≥ 30 μM, B > 10 μM and <30 μM, C ≥ 2 μM and ≤10 μM, D < 2 μM.

INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

EQUIVALENTS

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

What is claimed is:

1. A protease inhibitory compound represented by Formula XI: 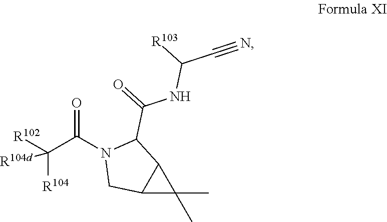
or a pharmaceutically acceptable salt, and/or a stereoisomer thereof, wherein:
R¹⁰⁴ is selected from the group consisting of 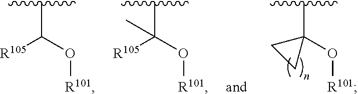
wherein R¹⁰⁵ is C1-C2alkyl, and n is 1, or 2;
R^{104d} is H; or R^{104a} and R^{104d}, together with the carbon atom to which they are attached, may be joined to form a 4-6 membered heterocyclyl;
R¹⁰¹ is selected from the group consisting of C₁-C₆alkyl, C₂-C₆alkenyl, C₃-C₆cycloalkyl, and C₃-C₆cycloalkenyl, wherein R¹⁰¹ may optionally be substituted by one, two, three, or four substituents each independently selected from R^{F};
or R¹⁰¹ and R¹⁰⁵ may be joined, together with the atoms to which they are attached, to form a 4-6 membered heterocyclyl, wherein the heterocyclyl may optionally be substituted by one, two, three, or four substituents each independently selected from the group consisting of halogen, and C1-C6alkyl;
R^{F} is selected from the group consisting of halogen, C₁-C₂alkyl, C₁-C₆alkoxy, C₃-C₆cycloalkyl, and cyano, wherein the alkyl may optionally be substituted by one or more halo;
R¹⁰² is selected from —NR^{m}(C═O)Rⁿ and 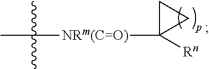
p is selected from 1 and 2;
R^{m} is H;
Rⁿ is selected from C₁-C₂alkyl and C₃-C₄cycloalkyl, wherein the alkyl or cycloalkyl is optionally substituted by one, two or three halo or —CF₃;
R¹⁰³ is selected from and 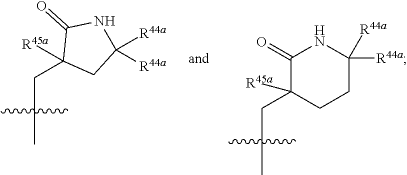
R^{44a} is each independently selected from the group consisting of hydrogen, —CH₃ and —CF₃, or two R^{44a} groups, together with the carbon to which they are attached, may be joined together to form a cyclopropyl, cyclobutyl, or oxetanyl; and
R^{45a} is H, CH₃ or halo.

2. The compound of claim 1, wherein Formula XI is represented by a formula selected from the group consisting of

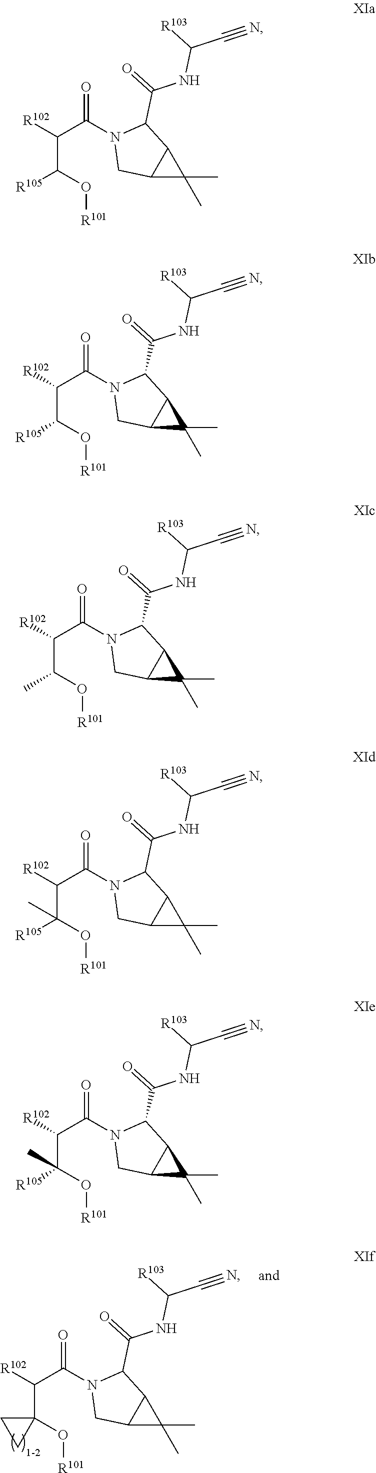

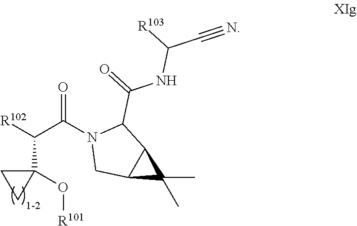

3. The compound of claim 1, wherein R¹⁰⁴ is 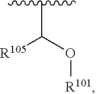
R¹⁰¹ is C₁-C₃alkyl or C₃-C₄cycloalkyl, and R¹⁰⁵ is C₁-C₂alkyl, wherein the R¹⁰¹ is optionally substituted by one or two substituents each independently selected from the group consisting of —F, —CF₃, —CH₃, —CN and cyclopropyl.

4. The compound of claim 3, wherein the 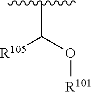
is selected from the group consisting of

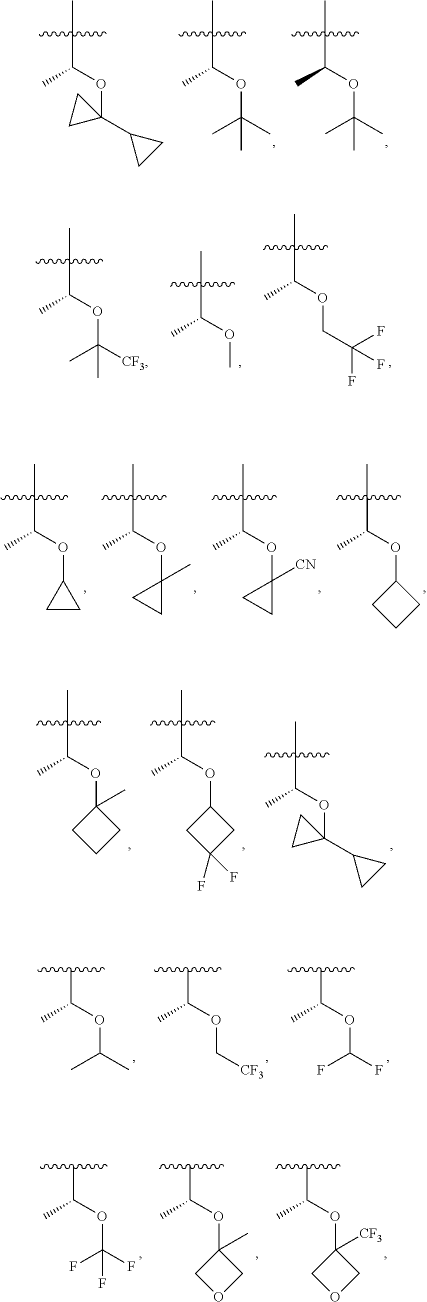

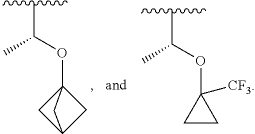

5. The compound of claim 1, wherein R¹⁰⁴ is 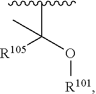
R¹⁰¹ is C₁-C₆alkyl or C₃-C₆cycloalkyl, and R¹⁰⁵ is C₁-C₂alkyl, wherein the R¹⁰¹ is optionally substituted by one or two substituents each independently selected from the group consisting of —F, —CF₃, —CH₃, —CN and cyclopropyl.

6. The compound of claim 5, wherein the 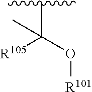
is

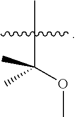

7. The compound of claim 1, wherein R¹⁰¹ is selected from the group consisting of:

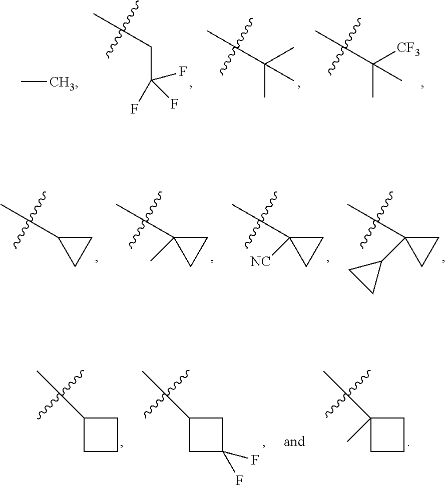

8. The compound of claim 1, wherein R¹⁰⁵ is —CH₃.

9. The compound of any one of claims 1-8, wherein R¹⁰² is —NH(C═O)CF₃.

10. The compound of any one of claims 1-8, wherein R¹⁰² is 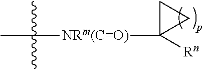
and p is selected from 1 and 2.

11. The compound of any one of claims 1-10, wherein R¹⁰³ is 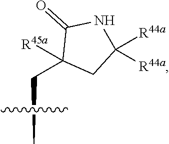
R^{44a} is each independently —CH₃, and R^{as} is H.

12. The compound of any one of claims 1-10, wherein R¹⁰³ is 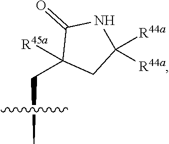
two R⁴⁴ groups, together with the carbon to which they are attached, are joined together to form a cyclopropyl, and R^{as1} is H.

13. The compound of any one of claims 1-10, wherein R¹⁰³ is 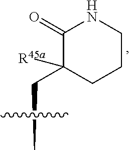
and R^{45a} is H.

14. The compound of any one of claims 1-9, wherein R¹⁰³ is 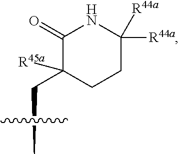
R^{44a} is H, and R^{45a} is H.

15. A protease inhibitory compound represented by Formula XII: 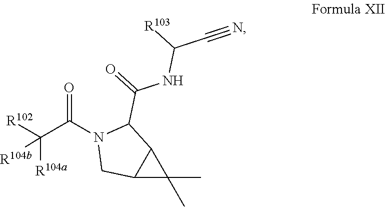
or a pharmaceutically acceptable salt, and/or a stereoisomer thereof, wherein:
R^{104a} is selected from the group consisting of 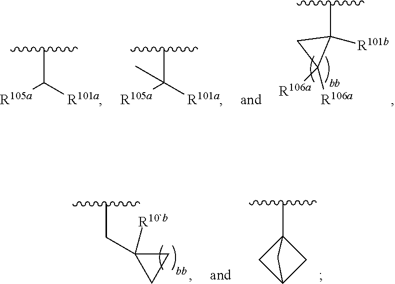
R^{105a} is C₁-C₂alkyl;
bb is selected from 1 and 2;
R^{106a} is each independently selected from H and halo;
R^{101a} and R^{101b} are each independently selected from the group consisting of H, halo, C₁-C₆alkyl, C₂-C₆alkenyl, C₃-C₆cycloalkyl, and C₃-C₆cycloalkenyl, wherein the alkyl, alkenyl, cycloalkyl, or cycloalkenyl may optionally be substituted by one, two, three, or four substituents each independently selected from the group consisting of halogen, C₁-C₆alkyl, C₁-C₆alkoxy, C₃-C₆cycloalkyl, phenyl, and pyridinyl, and cyano, wherein the alkyl may optionally be substituted by one or more halo;
R^{104b} represents H;
alternatively, R^{104a} and R^{104b}, together with the carbon atom to which they are attached, may be joined to form a C₃-C₆cycloalkyl, wherein the cycloalkyl ring may be optionally substituted by one, two, three, or four substituents each independently selected from R^{P};
wherein R^{P} is selected from the group consisting of halogen, C₁-C₆alkyl, C₁-C₆alkenyl, C₁-C₆alkoxy, C₃-C₆cycloalkyl, and cyano, wherein the alkyl may optionally be substituted by one or more halo;
R¹⁰² is selected from the group consisting of —NR^{m}(C═O)Rⁿ, 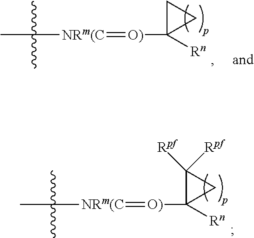
p is selected from 1 and 2;
R^{m} is H;
Rⁿ is selected from C₁-2alkyl and C₃-C₄cycloalkyl, wherein the alkyl or cycloalkyl is optionally substituted by one, two or three methyl, halo or —CF₃;
R^{pf} is each independently selected from H and halo;
R¹⁰³ is selected from 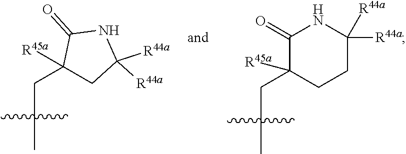
R^{44a} is each independently selected from the group consisting of hydrogen, —CH₃ and —CF₃, or two R^{44a} groups, together with the carbon to which they are attached, may be joined together to form a cyclopropyl, cyclobutyl, or oxetanyl; and
R^{45a} is each independently selected from the group consisting of H, CH₃ and halo.

16. The compound of claim 15, wherein Formula XII is represented by a formula selected from the group consisting of

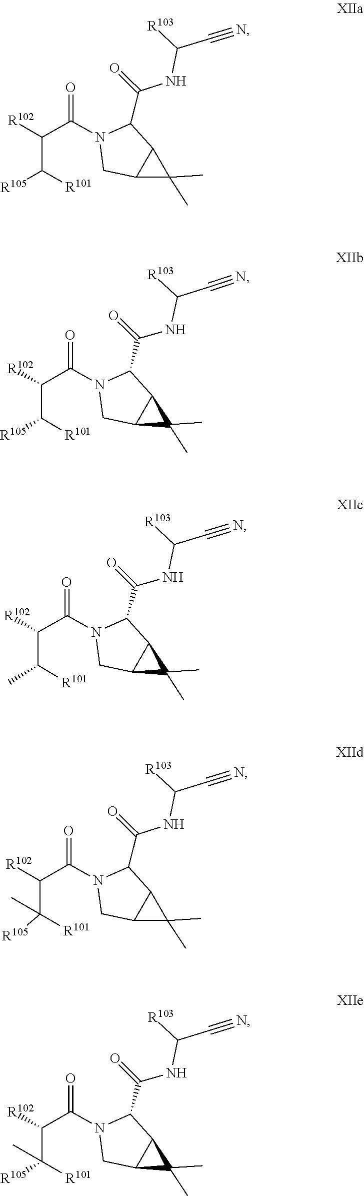

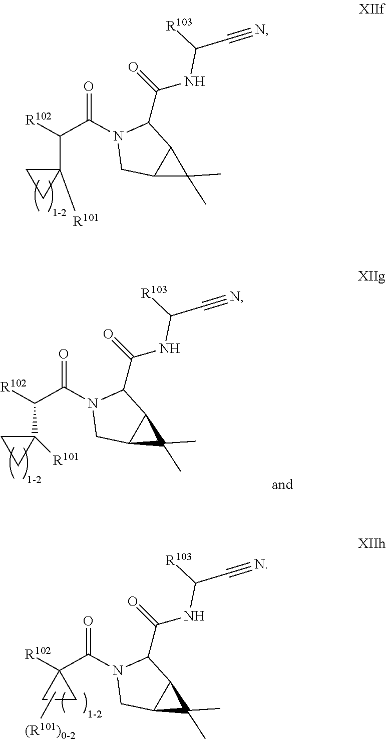

17. The compound of claim 15, wherein R^{104a} is 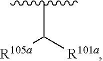
R^{101a} is C₁-C₃alkyl or C₃-C₄cycloalkyl, and R^{105a} is C₁-C₂alkyl, wherein the R¹⁰¹ is optionally substituted by one or two substituents each independently selected from the group consisting of —F, —CF₃, —CH₃, —CN and cyclopropyl.

18. The compound of claim 15, wherein the 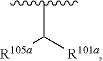
is selected from the group consisting of

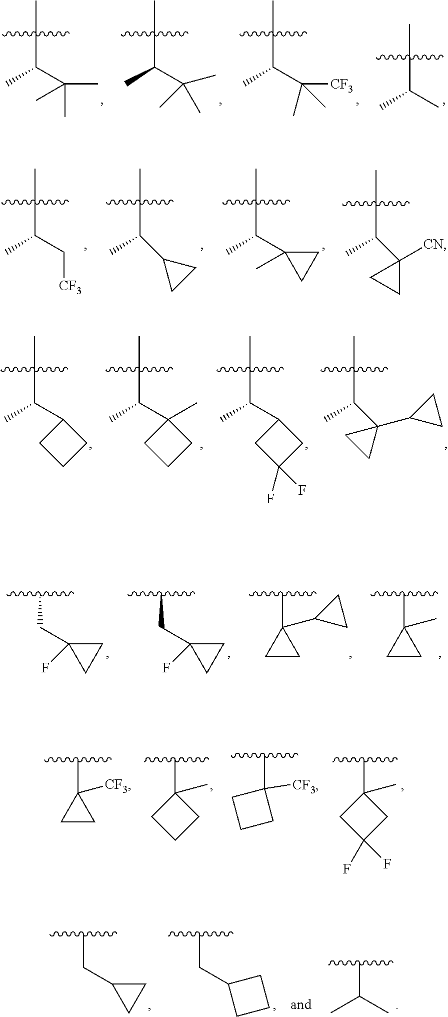

19. The compound of claim 15, wherein R^{104a} is 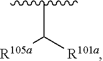
R^{101a} is C₁-C₆alkyl or C₃-C₆cycloalkyl, and R^{105a} is C₁-C₂alkyl, wherein the R¹⁰¹ is optionally substituted by one or two substituents each independently selected from the group consisting of —F, —CF₃, —CH₃, —CN and cyclopropyl.

20. The compound of claim 15, wherein the 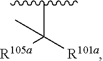
is selected from the group consisting of % and

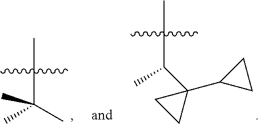

21. The compound of claim 15, wherein R^{101a} is selected from the group consisting of:

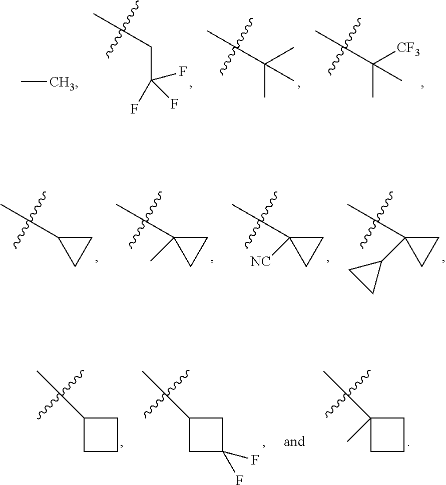

22. The compound of claim 15, wherein R^{105a} is —CH₃.

23. The compound of any one of claims 15-22, wherein R¹⁰² is —NH(C═O)CF₃.

24. The compound of any one of claims 15-22, wherein R¹⁰² 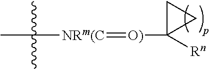
and p is selected from 1 and 2.

25. The compound of any one of claims 15-24, wherein R¹⁰² is 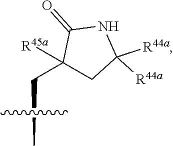
R^{44a} is each independently —CH₃, and R^{as} is H.

26. The compound of any one of claims 15-24, wherein R¹⁰³ is 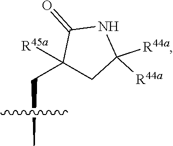
two R⁴⁴ groups, together with the carbon to which they are attached, are joined together to form a cyclopropyl, and R^{45a} is H.

27. The compound of any one of claims 15-24, wherein R¹⁰³ is 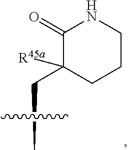
and R^{as} is H.

28. The compound of any one of claims 15-24, wherein R¹⁰³ is 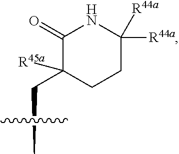
R^{44a} is H, and R^{45a} is H.

29. A formula I: 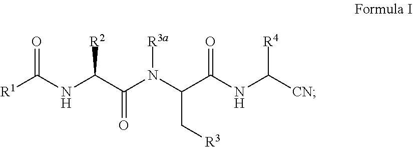
or a pharmaceutically acceptable salt or stereoisomer thereof, wherein
R¹ is selected from the group consisting of —CF₃, C₁-C₆alkyl, —C₃-C₆cycloalkyl, —C₁-C₆alkoxy, —O—C₆-C₁₀cycloalkyl, —O-C₃cycloalkyl, —O-C₄cycloalkyl, —O-C₅cycloalkyl, —O—C(R¹⁰)₂-C₆-C₁₀cycloalkyl, —O—C(R¹⁰)₂-phenyl, —O-phenyl, phenyl, —O-(4-1 membered heterocyclyl), —O(CH₂CH₂)ₛ—OR₁₁, —C(R¹²)₂—O—(CH₂CH₂)ₛ—OR¹¹, and 5-6 membered heteroaryl; wherein R¹ may optionally be substituted by one or more substituents each selected from R⁵; s is selected from 1 and 2;
R¹⁰ is selected from hydrogen and deuterium;
R¹¹ and R¹² are each independently hydrogen or C₁-C₃alkyl optionally substituted by one or more halogens;
R² is selected from the group consisting of C₁-C₆alkyl, CH₂-R²², and C₃-C₆cycloalkyl;
wherein R² or R²² may optionally be substituted by one, two, or three substituents each selected from R⁵;
wherein when R¹ is —CF₃, R² is not t-butyl;
R²² is selected from the group consisting of phenyl, naphthyl, and 8-10 membered bicyclic heteroaryl;
R^{3a} is selected from hydrogen and C₁-C₃alkyl;
R³ is selected from the group consisting of C₁-C₆alkyl, C₃-C₆cycloalkyl, phenyl and 4 6 heterocyclyl and 5-6 membered heteroaryl; wherein R³ may optionally be substituted by one, two, or three substituents each selected from R⁵; or R³ is —Si(CH₃)₃;
or R^{3a} and R³, together with the atoms to which they are attached, may be joined together to form a 4-6 membered monocyclic, 6-10 membered fused bicyclic, or 6-10 membered spirocyclic heterocycle which may optionally be substituted by one, two or three substituents each selected from R⁵;
R⁴ is 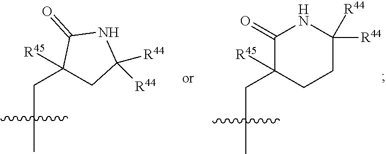
R⁴⁴ is each independently selected from the group consisting of hydrogen, —CH₃ and —CF₃, or two R⁴⁴ groups, together with the carbon to which they are attached, may be joined together to form a cyclopropyl, cyclobutyl, or oxetanyl;
R⁴⁵ is selected from hydrogen and C₁-C₃alkyl; or one R⁴⁴ and R^{°S}, together with the atoms to which they are attached, may be joined together to form a 4-6 membered carbocycle;
R⁵ is independently selected for each occurrence from the group consisting of halogen, hydroxyl, cyano, —N(R⁵⁵)₂, —N(R⁵⁵)C(O)R⁵⁵, —C(O)N(R⁵⁵)₂, —OPO(OH)₂, —CO₂H, —SO₂CH₃ —CF₃, —CHF₂, C₁-C₆alkyl, C₁-C₆alkoxy, and phenyl; wherein C₁-C₆alkyl, C₁-C₆alkoxy and phenyl may optionally be substituted by one or more substituents each independently selected from the group consisting of halogen and hydroxyl; and wherein two geminal R⁵ groups, together with the carbon to which they are attached, may be joined together to form a cyclopropyl or cyclobutyl; and
R⁵⁵ is selected from hydrogen and C₁-C₃alkyl, wherein the alkyl is optionally substituted by one, two or three halogens;
or two R⁵⁵ groups, together with the atoms to which they are attached, may be joined together to form a 4-6 membered heterocyclyl, wherein the heterocycicyl may optionally be substituted by one, two or three substituents each independently selected from the group consisting of halogen, hydroxyl and C₁-C₃alkyl (optionally substituted by one or more halogens).

30. The compound of claim 29, wherein R¹ is selected from the group consisting of:

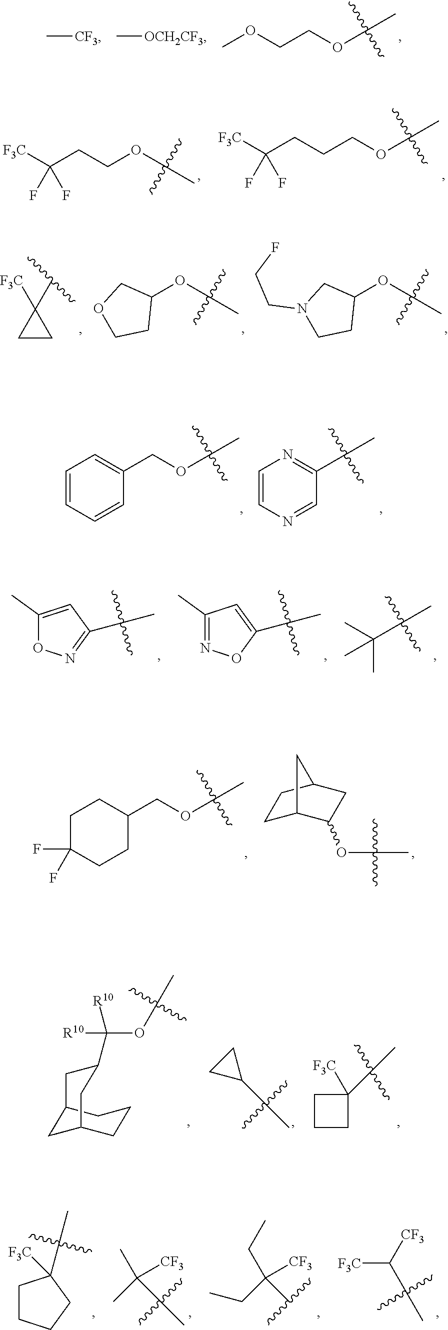

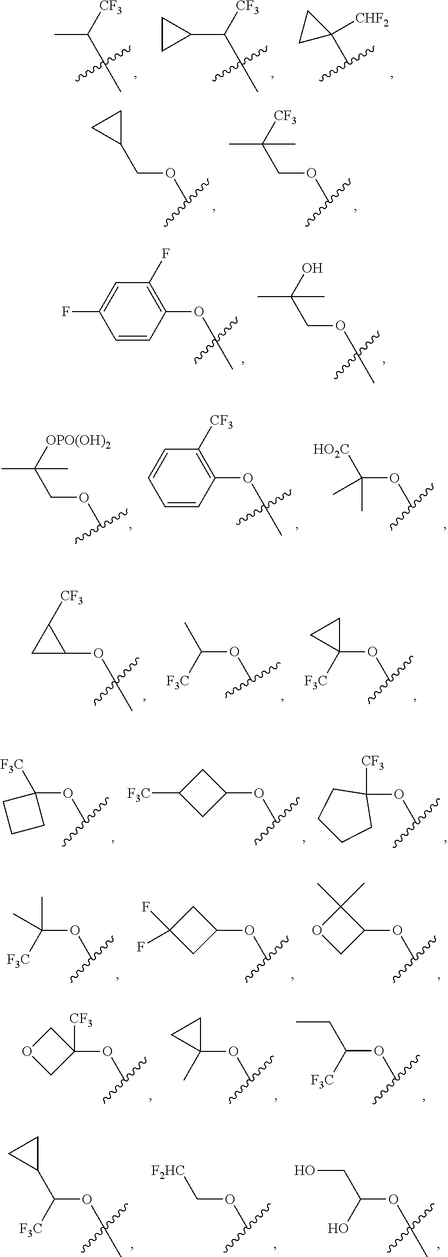

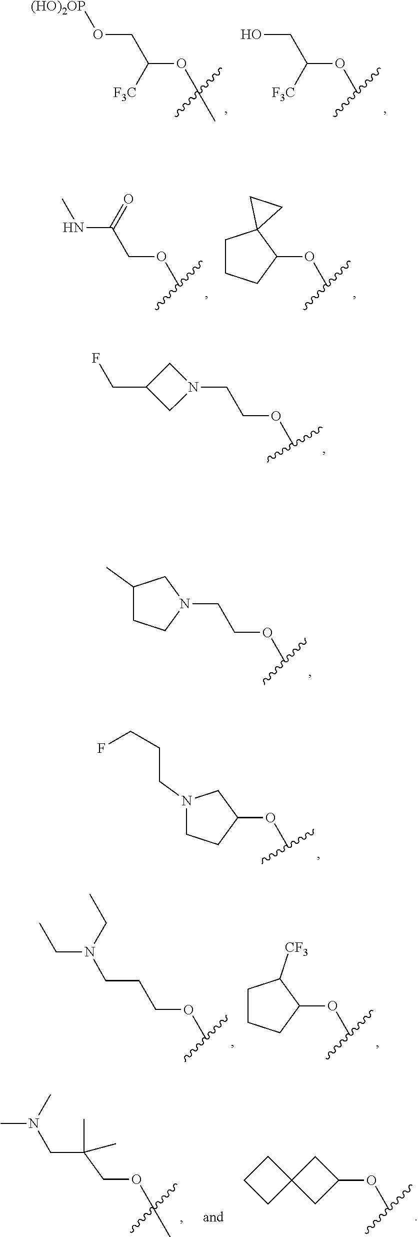

31. The compound of claim 29 or 30, wherein R¹ is selected from the group consisting of:

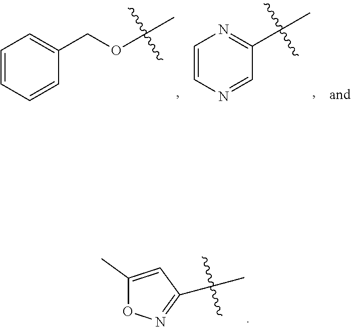

32. The compound of any one of claims 29-31, wherein R² is selected from the group consisting of:

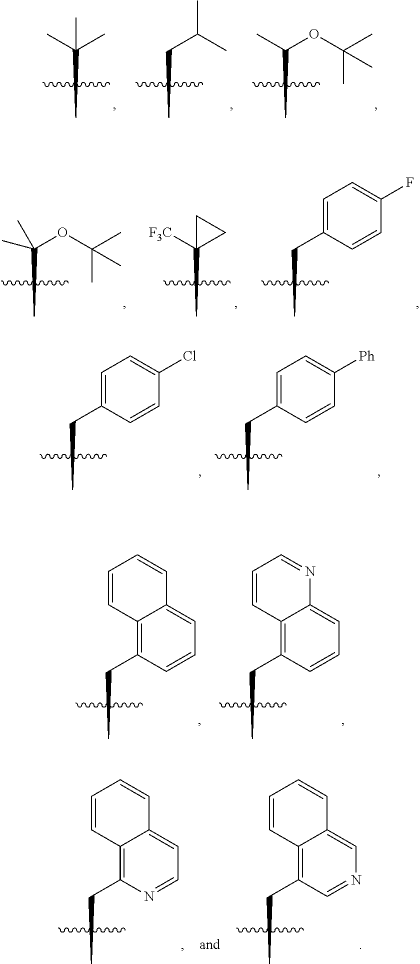

33. The compound of any one of claims 29-32, wherein R² is selected from the group consisting of:

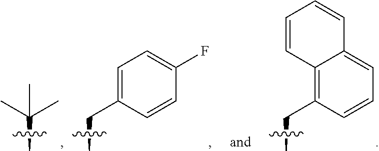

34. The compound of any one of claims 29-33, wherein R^{3a} is hydrogen.

35. The compound of any one of claims 29-34, wherein R³ is selected from the group consisting of:

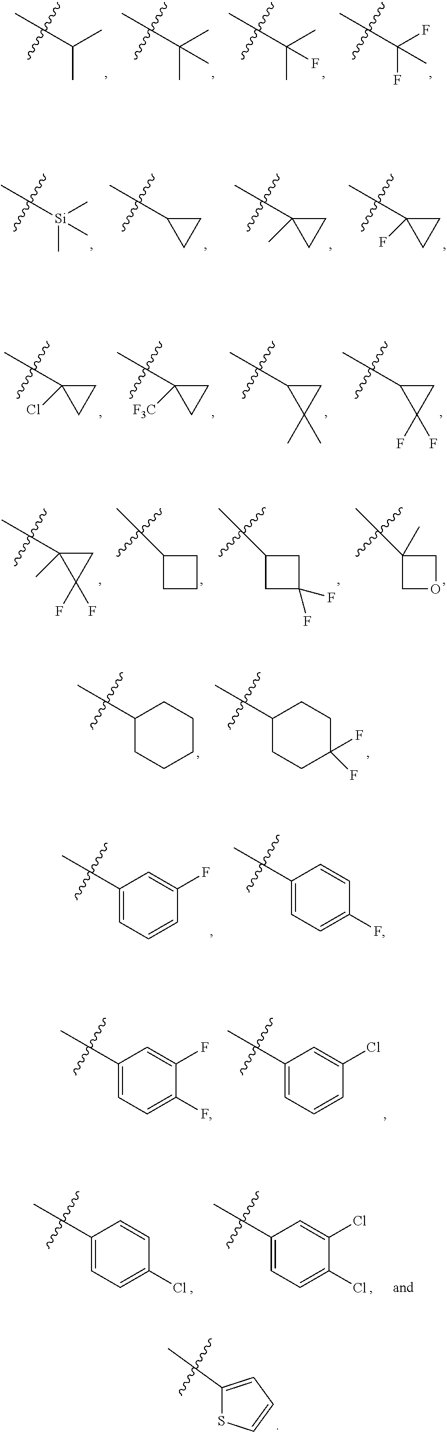

36. The compound of any one of claims 29-35, wherein R³ is selected from the group consisting of:

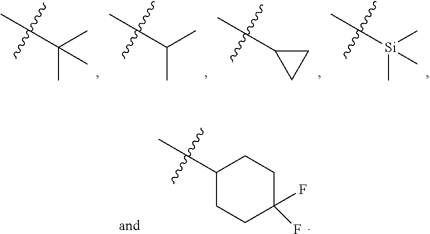

37. The compound of any one of claims 29-36, wherein R^{3a} and R³, together with the atoms to which they are attached, join together to form a 4-6 membered monocyclic, 6-10 membered fused bicyclic, or 6-10 membered spirocyclic heterocycle.

38. The compound of claim 29 or 37, wherein the compound is represented by a formula selected from the group consisting of: 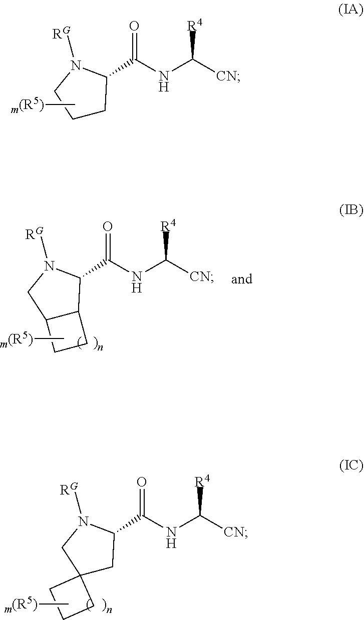
wherein m is selected from 0, 1, 2 and 3; n is selected from 0, 1, 2 and 3; and
R^{G} is

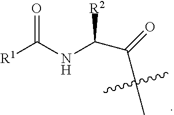

38. The compound of claim 29 or 37, wherein R^{3a} and R³, together with the atoms to which they are attached, join together to form a 4-6 membered monocyclic, 6-10 membered fused bicyclic, or 6-10 membered spirocyclic heterocycle selected from the group consisting of: 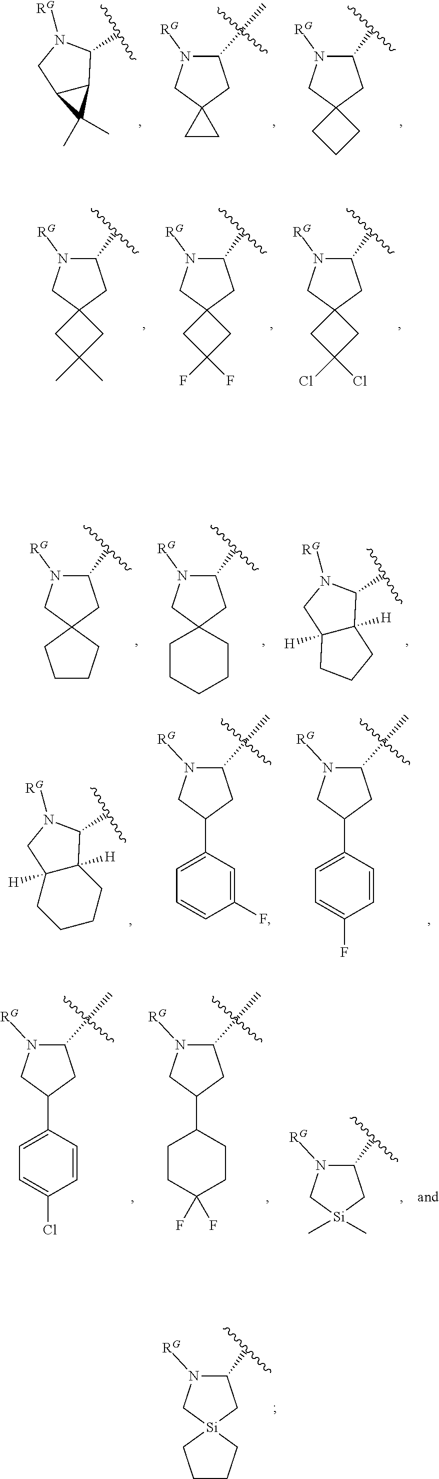
wherein R^{G} is

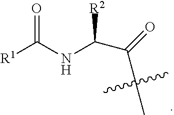

39. The compound of any one of claims 29-38, wherein R⁴ is selected from the group consisting of:

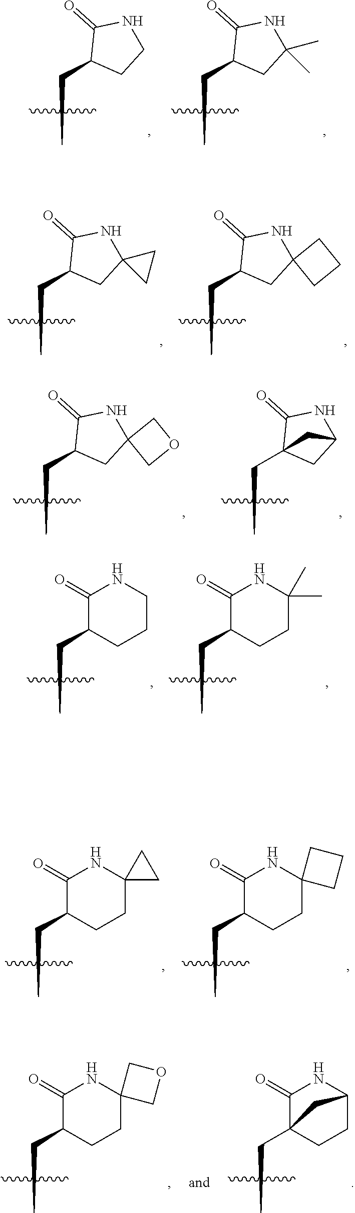

40. The compound of claim 29, wherein R⁵ is selected from the group consisting of chloro, fluoro, —CF₃ —CH₃ and phenyl.

41. A protease inhibitory compound represented by Formula II or Formula III: 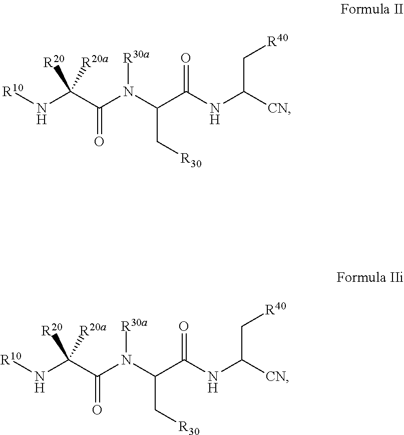
or a pharmaceutically acceptable salt or stereoisomer thereof, wherein:
R¹⁰ is selected from the group consisting of hydrogen, —CH₂CF₃, —CH₂CF₂H, —CH(CF₃)₂, —CH(CF₃)CH₃, —C(CH₃)(CF₃)₂, —C(O)NR^{a}R¹⁰⁰, C₃-C₆cycloalkyl, phenyl, and 5-6 membered heteroaryl, wherein phenyl and 5-6 membered heteroaryl may optionally be substituted by or more substituents each independently selected from R⁵⁰;
R^{a} is hydrogen or C₁-C₃alkyl;
R¹⁰⁰ is selected from the group consisting of hydrogen, C₁-C₆alkyl, C₃-C₁₀cycloalkyl, —C(R¹⁰¹)₂-C₆-C₁₀cycloalkyl, phenyl, —C(R¹⁰¹)₂-phenyl, 4-6 membered heterocyclyl, and 5-6 membered heteroaryl; wherein R¹⁰⁰ may optionally be substituted by one or more substituents each selected from R⁵⁰;
or R^{a} and R¹⁰⁰, together with the nitrogen to which they are attached, may be joined together to form a 4-6 membered monocyclic or a 6-10 membered spirocyclic heterocyclyl optionally substituted by one or more halogens;
R¹⁰¹ is hydrogen or deuterium;
R²⁰ is selected from the group consisting of C₁-C₆alkyl CH₂-R²²⁰; and C₃-C₆cycloalkyl,
wherein R²⁰ or R²²⁰ may optionally be substituted by one, two, or three substituents each selected from R⁵⁰;
R is H; or R²⁰ and R^{20a}, together with the carbon to which they are attached, may be joined together to form a C₃-C₆cycloalkyl or 4-6 membered monocyclic heterocyclyl;
R²²⁰ is selected from the group consisting of phenyl, naphthyl, and 8-10 membered bicyclic heteroaryl;
R^{30a} is hydrogen or C₁-C₃alkyl;
R³⁰ is selected from the group consisting of C₁-C₆alkyl, C₃-C₆cycloalkyl, phenyl, 4-6 heterocyclyl and 5-6 membered heteroaryl; wherein R³⁰ may optionally be substituted by one, two, or three substituents each selected from R⁵⁰; or R³⁰ is —Si(CH₃)₃;
or R and R³⁰, together with the atoms to which they are attached, may be joined together to form a 4-6 membered monocyclic, 6-10 membered fused bicyclic, or 6-10 membered spirocyclic heterocyclyl which may optionally be substituted by one, two or three substituents each selected from R⁵⁰;
R⁴⁰ is 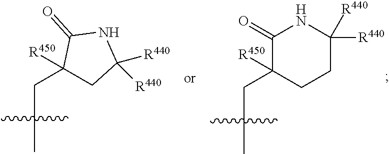
R⁴⁴⁰ is each independently selected from the group consisting of hydrogen, —CH₃ and —CF₃, or two R⁴⁴⁰ groups, together with the carbon to which they are attached, may be joined together to form a cyclopropyl, cyclobutyl, or oxetanyl;
R⁴⁵⁰ is hydrogen; or one R⁴⁴⁰ and R⁴⁵⁰, together with the atoms to which they are attached, may be joined together to form a 4-6 membered carbocycle;
R⁵⁰ is independently selected for each occurrence from the group consisting of halogen, hydroxyl, cyano, —N(R⁵⁵⁰)₂, —N(R⁵⁵⁰)C(O)R⁵⁵⁰, —C(O)N(R⁵⁵⁰)₂, —OPO(OH)₂, —CO₂H, —SO₂CH₃ —CF₃, —CHF₂, C₁-C₆alkyl, C₁-C₆alkoxy, and phenyl; wherein C₁-C₆alkyl, C₁-C₆alkoxy and phenyl may optionally be substituted by one or more substituents each independently selected from the group consisting of halogen and hydroxyl; and
R⁵⁵⁰ is hydrogen or C₁-C₃alkyl optionally substituted by one, two or three halogens;
or two R⁵⁵ groups, together with the atoms to which they are attached, may be joined together to form a 4-6 membered heterocyclyl, wherein the heterocycicyl may optionally be substituted by one, two or three substituents each independently selected from halogen, hydroxyl and C₁-C₃alkyl (optionally substituted by one or more halogens).

42. The compound of claim 15, wherein the compound is represented by a formula selected from the group consisting of:

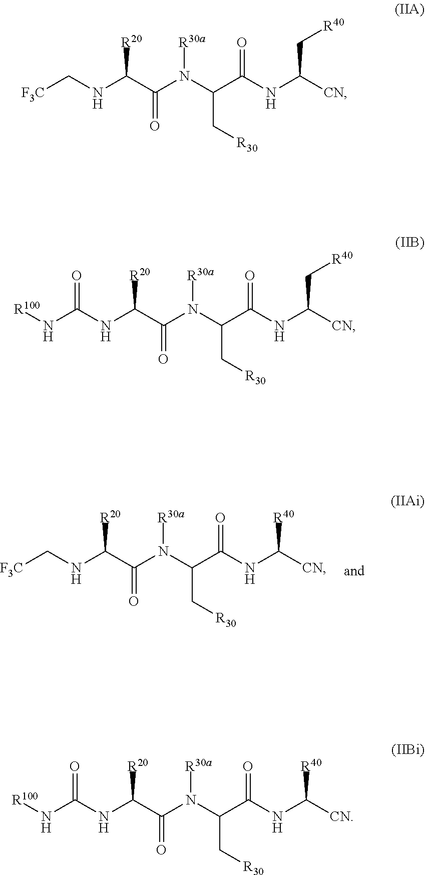

43. The compound of claim 41 or 42, wherein R¹⁰ is selected from the group consisting of:

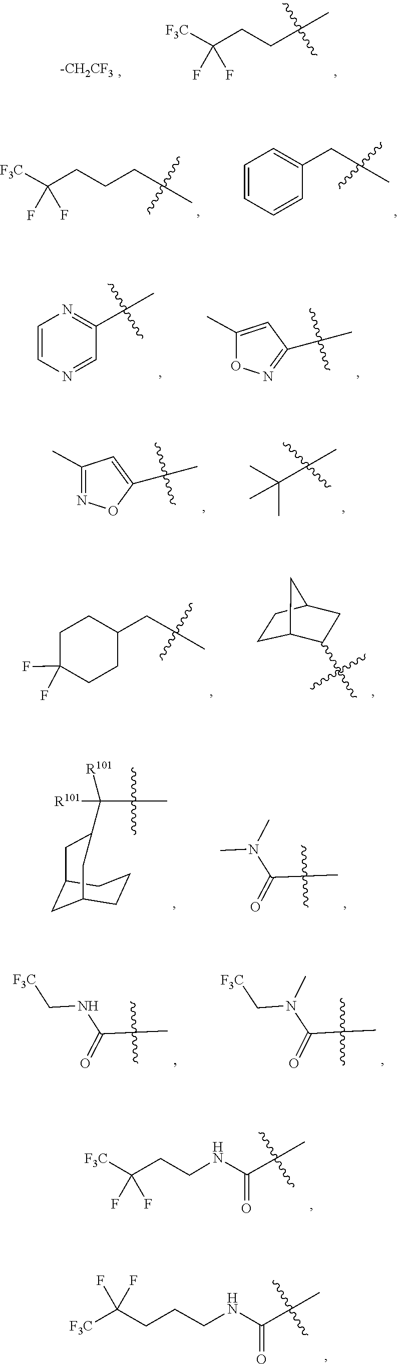

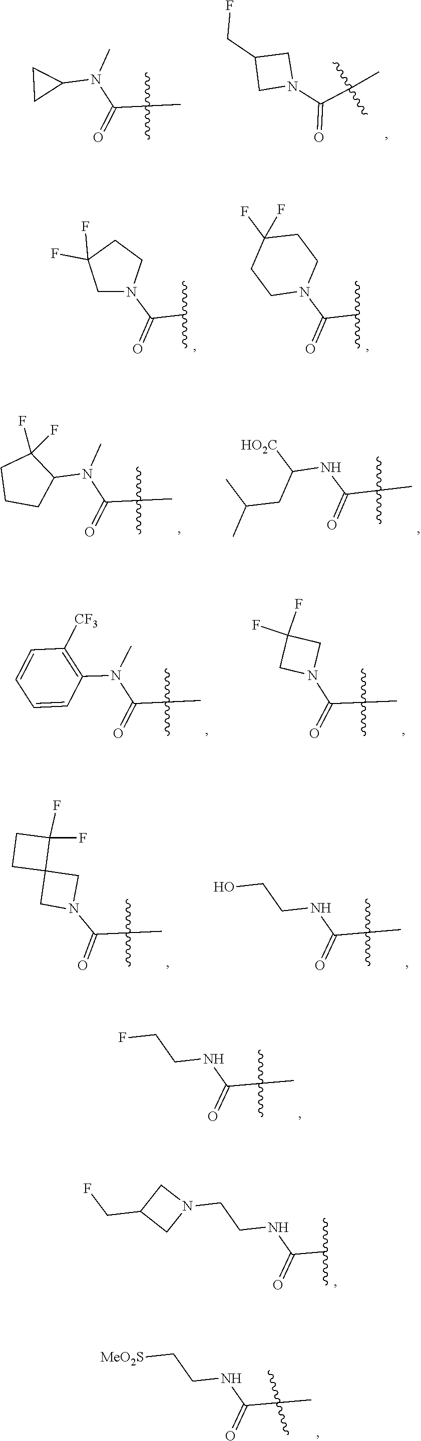

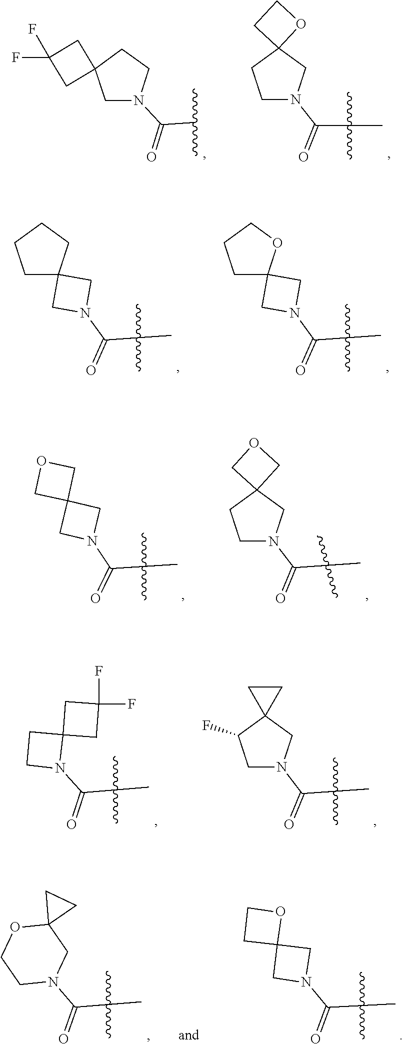

44. The compound of claim 41, wherein R¹⁰ is 5-6 membered heteroaryl or 4-6 membered heterocyclyl.

45. The compound of claim 41 or 44, wherein R¹⁰ is selected from the group consisting of: 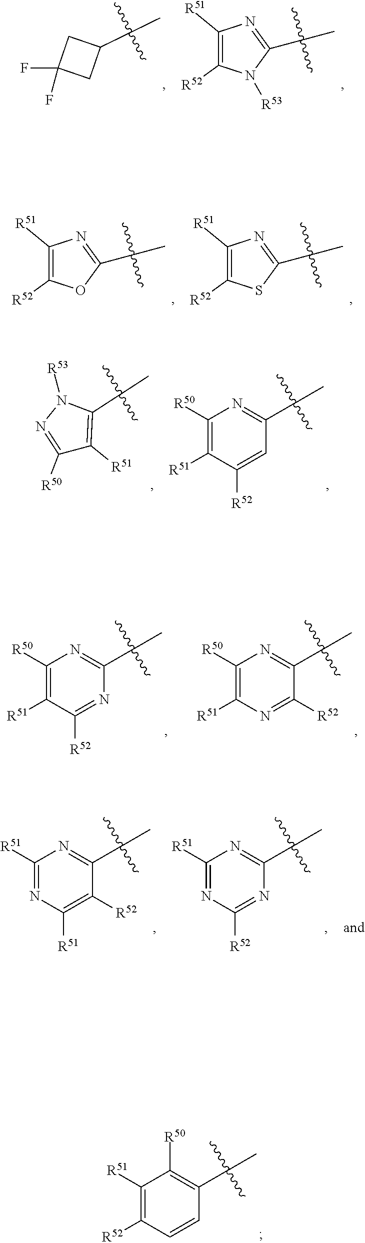
wherein R⁵⁰, R⁵¹ and R⁵² are each independently selected from the group consisting of chloro, fluoro, C₁-C₃alkyl and C₁-C₃alkoxy, wherein C₁-C₃alkyl and C₁-C₃alkoxy may optionally be substituted by one, two or three halogens; and wherein R⁵³ is hydrogen or C₁-C₃alkyl optionally substituted by one, two or three halogens.

46. The compound of any one of claims 41-45, wherein R²⁰ is selected from the group consisting of:

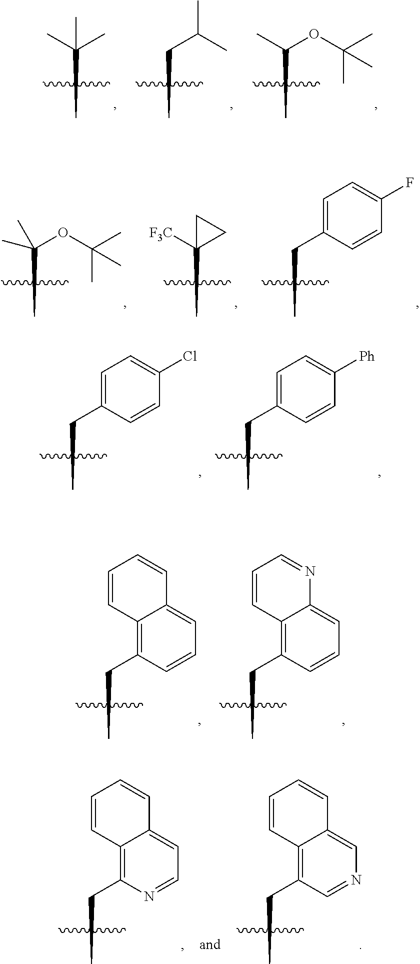

47. The compound of any one of claims 41-46, wherein R is hydrogen.

48. The compound of any one of claims 41-47, wherein R³⁰ is selected from the group consisting of:

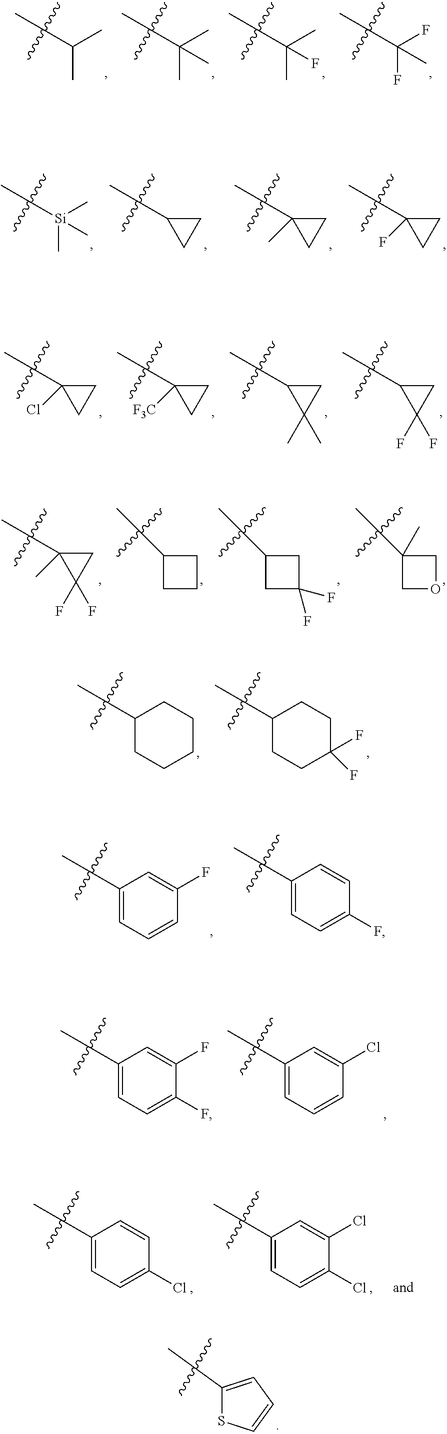

49. The compound of claim 48, wherein R³ and R³⁰, together with the atoms to which they are attached, join together to form a 4-6 membered monocyclic, 6-10 membered fused bicyclic, or 6-10 membered spirocyclic heterocyclyl.

50. The compound of any one of claims 41-49, wherein the compound is represented by a formula selected from the group consisting of: 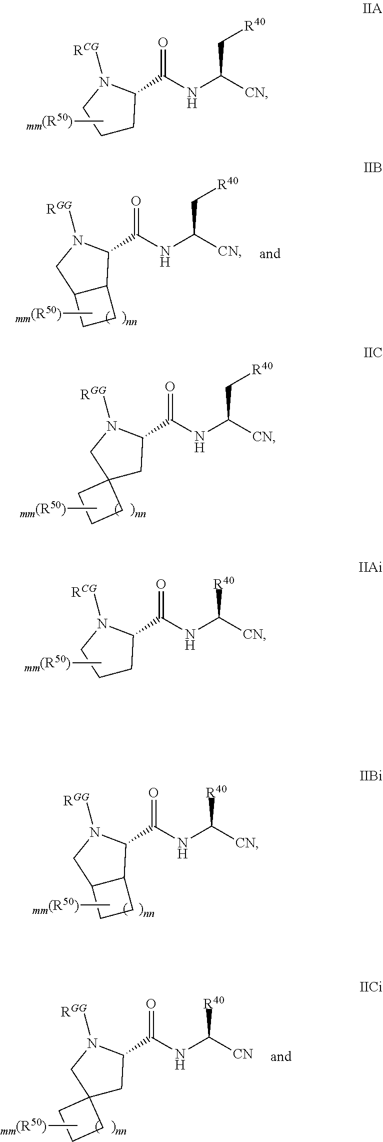
wherein mm is 0, 1 or 2; nn is 0, 1, 2 or 3; and
R^{GG} is

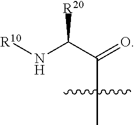

51. The compound of any one of claims 41-50, wherein R³ and R³⁰, together with the atoms to which they are attached, join together to form a 4-6 membered monocyclic, 6-10 membered fused bicyclic, or 6-10 membered spirocyclic heterocycle selected from the group consisting of: 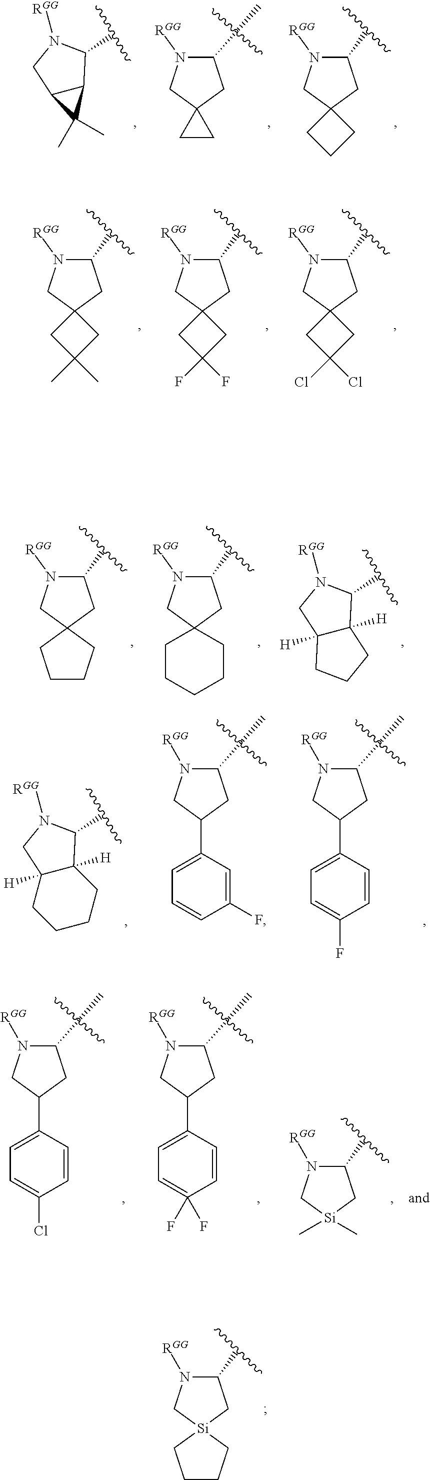
wherein R^{GG} is

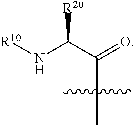

52. The compound of any one of claims 41-51, wherein R⁴⁰ is selected from the group consisting of:

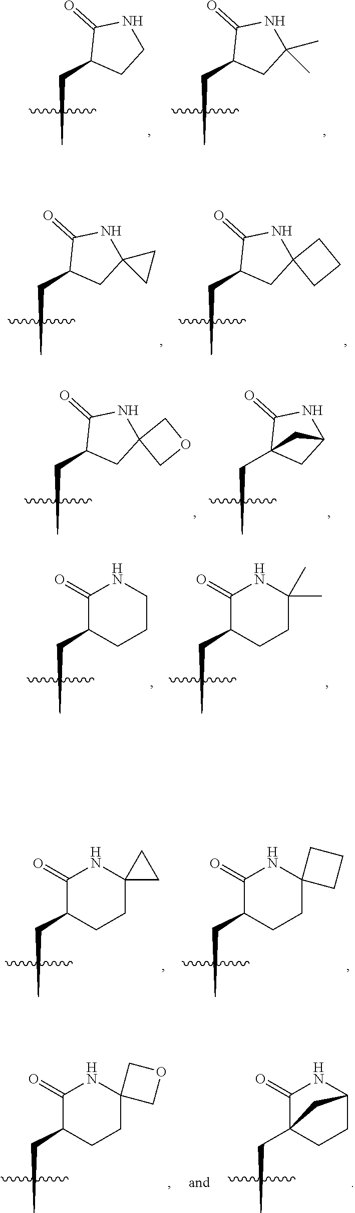

53. A protease inhibitory compound represented by Formula III: 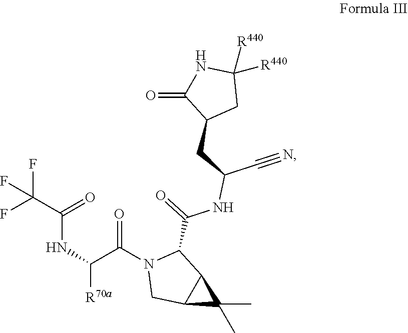
or a pharmaceutically acceptable salt or stereoisomer thereof, wherein
R⁴⁴⁰ is each independently selected from the group consisting of hydrogen, —CH₃ and —CF₃,
or two R⁴⁴⁰ groups, together with the carbon to which they are attached, may be joined together to form a cyclopropyl, cyclobutyl, or oxetanyl;
R^{70a} is selected from the group consisting of C₁₋₃alkylene-C₃-C₅cycloalkyl, C₁₋₆alkyl, C₃-C₅cycloalkyl, C₁₋₃alkyl-O— R⁵⁰¹ and a 4-6 membered heterocyclyl having one ring oxygen; wherein R^{70a} may optionally be substituted by one, two, three substituents each independently selected from R⁵⁰⁰;
R⁵⁰¹ is selected from the group consisting of C₁₋₆alkyl, C₁₋₆alkenyl, phenyl, C₃-C₆cycloalkyl, 4-6 membered heterocyclyl having one ring oxygen and (5-6 membered heteroaryl) wherein R⁵⁰¹ may be optionally substituted by one two, three or more substituents each independently selected from the group consisting of halo, cyano, methyl, ethyl, C₁₋₆alkoxy, CF₃, —CHF₂, —CH₂—CF₃ and C₃₋₆cycloalkyl; and
R⁵⁰⁰ is independently selected for each occurrence from the group consisting of halogen, cyano, —CF₃, —CHF₂, C₁-C₂alkyl, and C₂₋₆alkenyl.

54. A compound selected from the group consisting of: 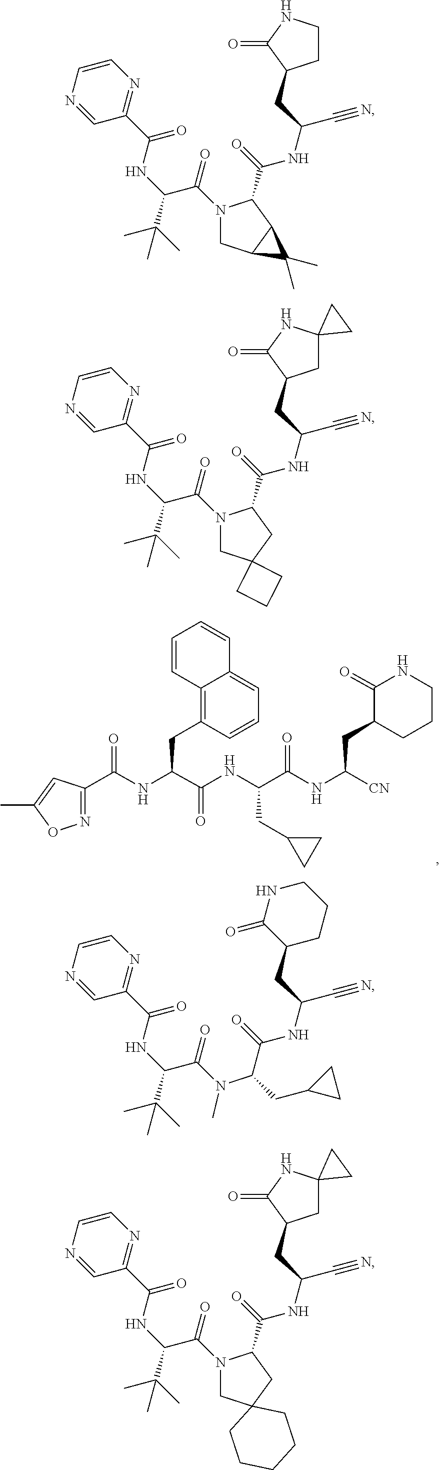 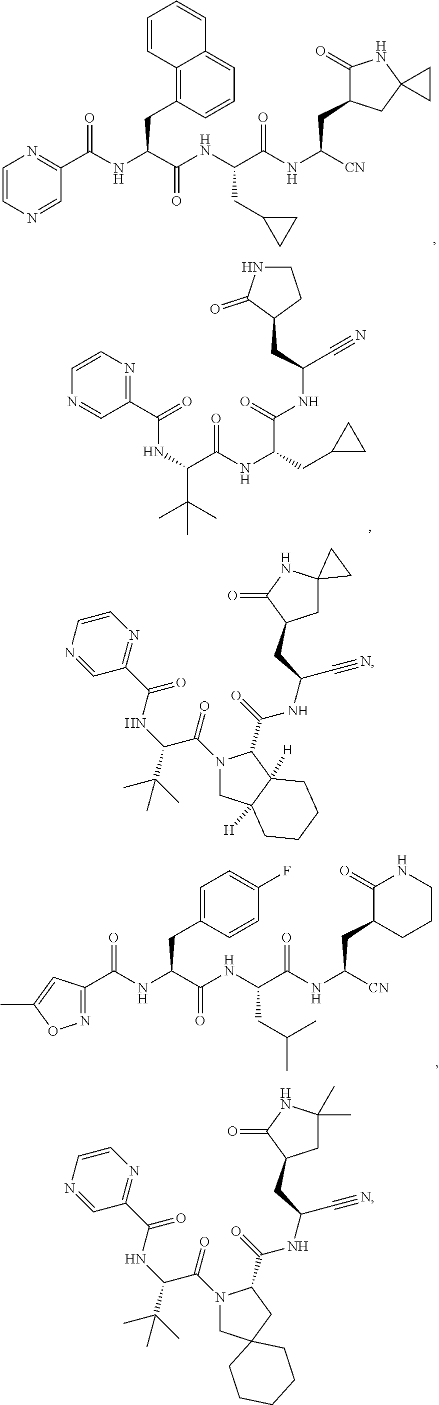 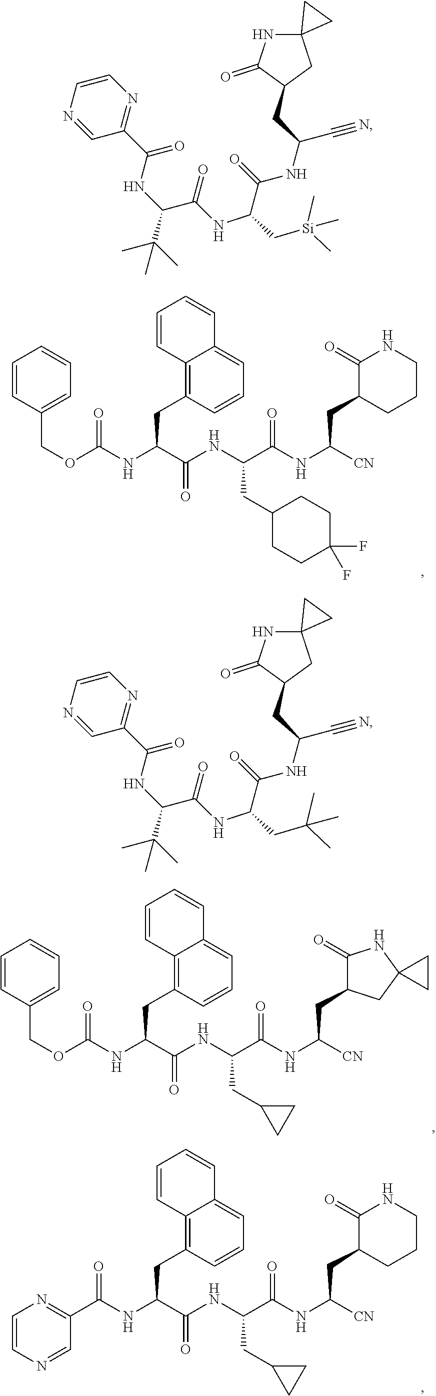 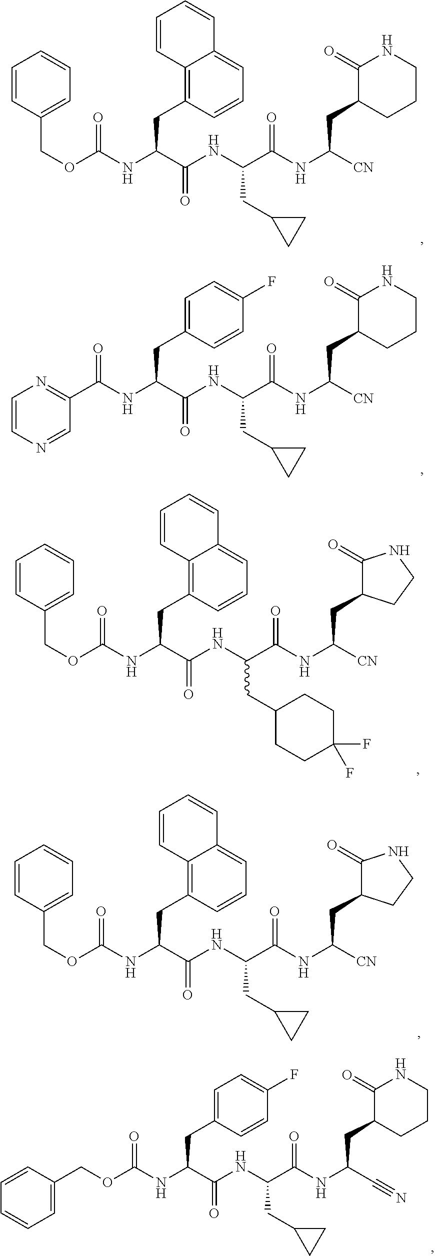 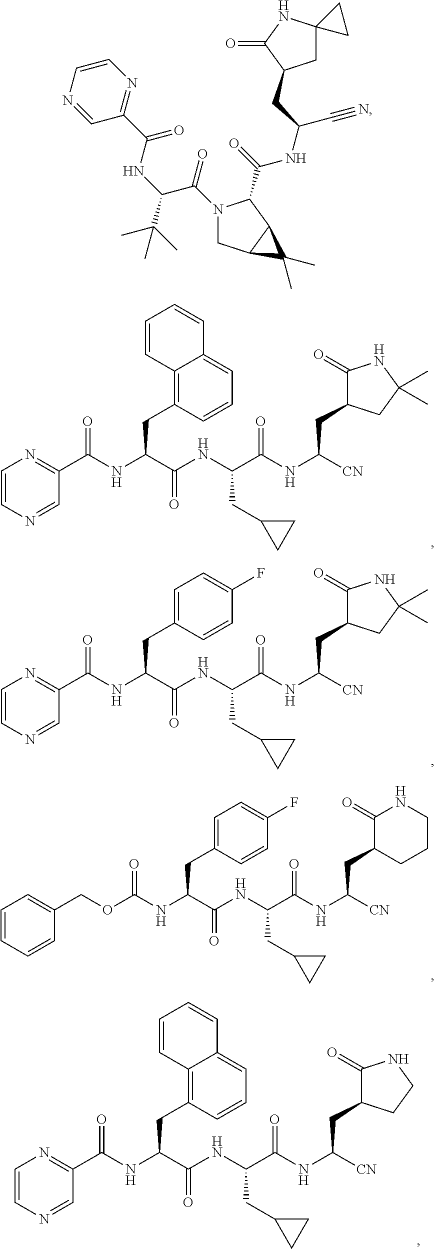 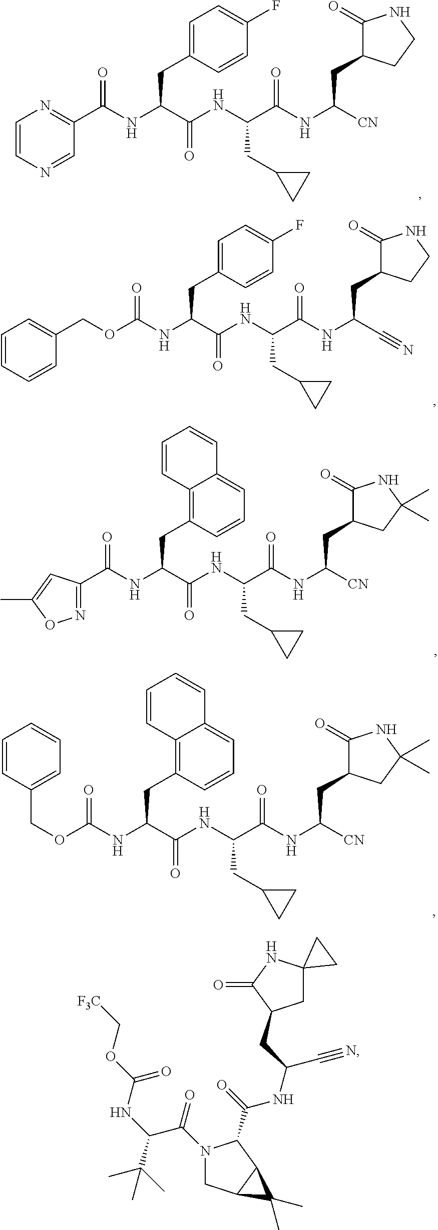 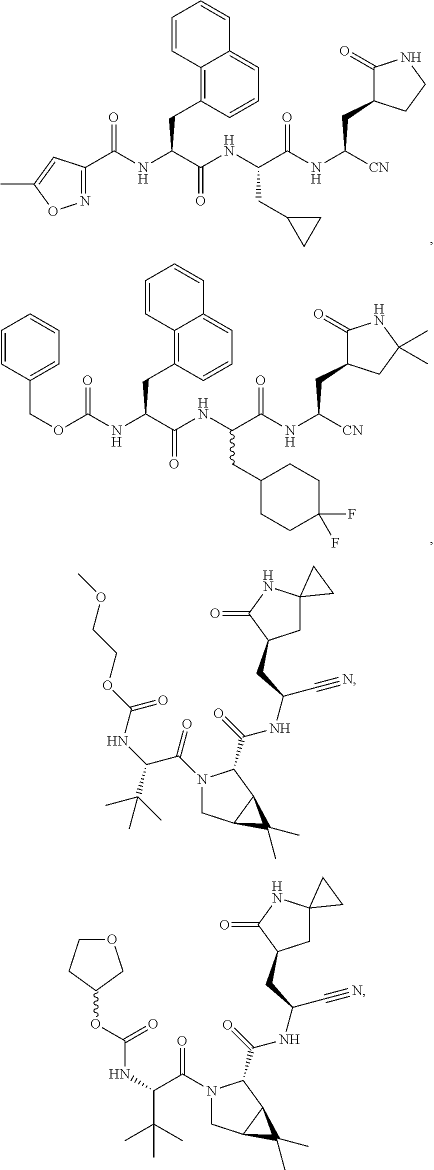 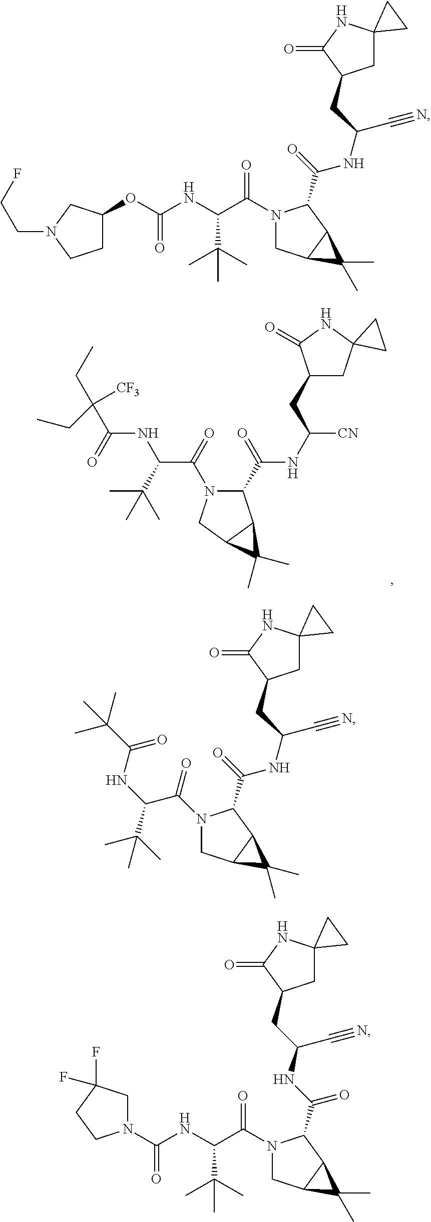 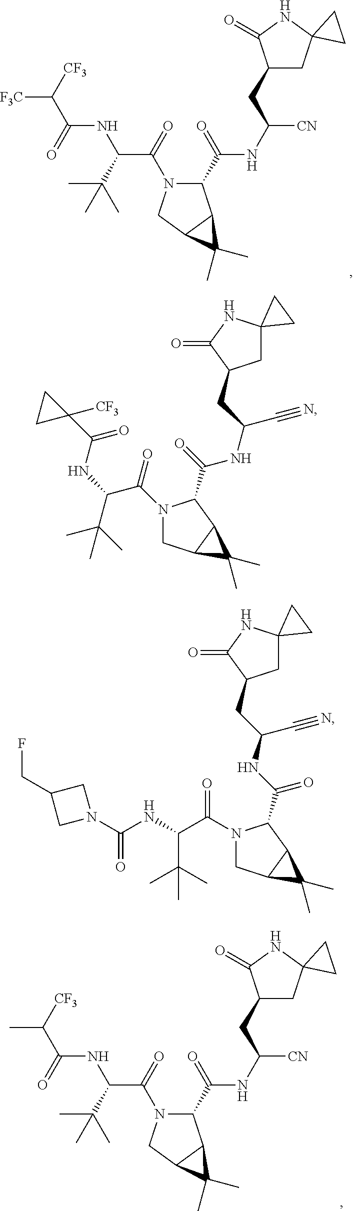 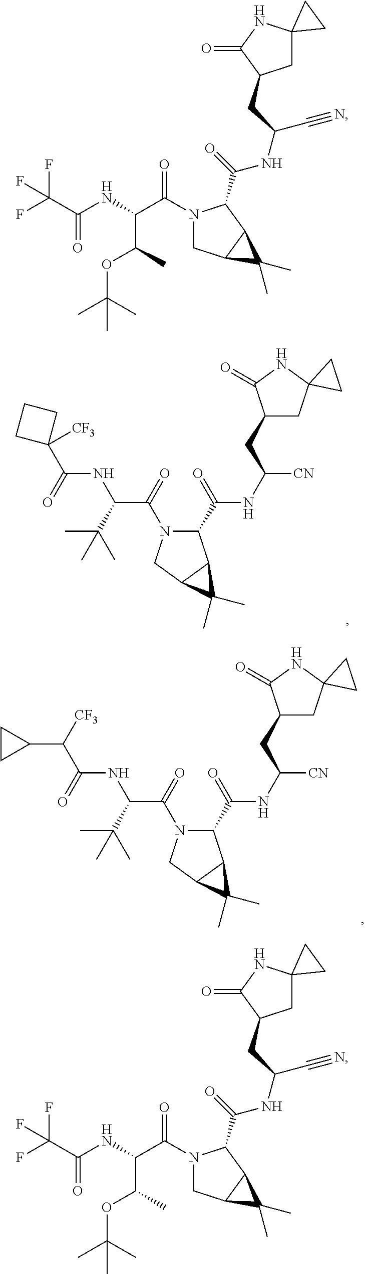 [SMILES: C[C@@H](OC(C)(C)C)[C@H](NC(=O)C(F)(F)F)C(=O)N1C[C@H]2[C@@H]([C@H]1C(=O)N[C@H](C#N)C[C@@H]1CC3(CC3)NC1=O)C2(C)C ; CC(C)(C)[C@H](NC(=O)C1(C(F)(F)F)CCC1)C(=O)N1C[C@H]2[C@@H]([C@H]1C(=O)N[C@H](C#N)C[C@@H]1CC3(CC3)NC1=O)C2(C)C ; CC(C)(C)[C@H](NC(=O)C(C1CC1)C(F)(F)F)C(=O)N1C[C@H]2[C@@H]([C@H]1C(=O)N[C@H](C#N)C[C@@H]1CC3(CC3)NC1=O)C2(C)C ; C[C@H](OC(C)(C)C)[C@H](NC(=O)C(F)(F)F)C(=O)N1C[C@H]2[C@@H]([C@H]1C(=O)N[C@H](C#N)C[C@@H]1CC3(CC3)NC1=O)C2(C)C] 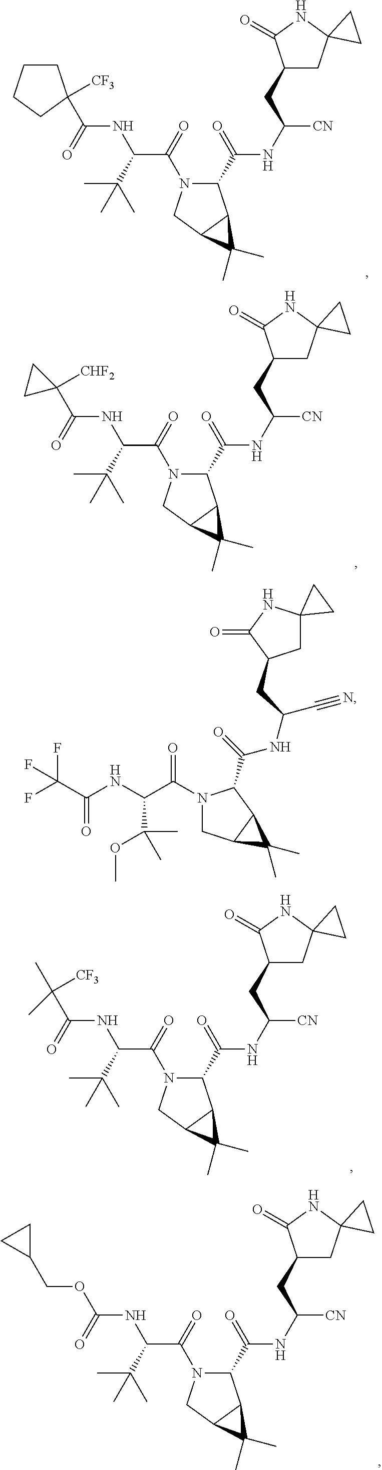 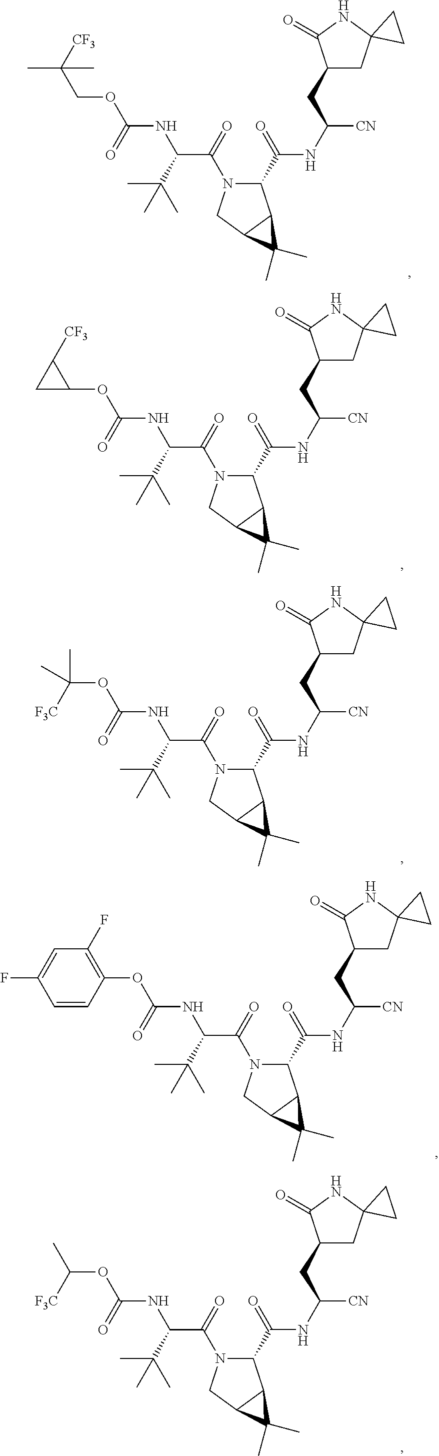 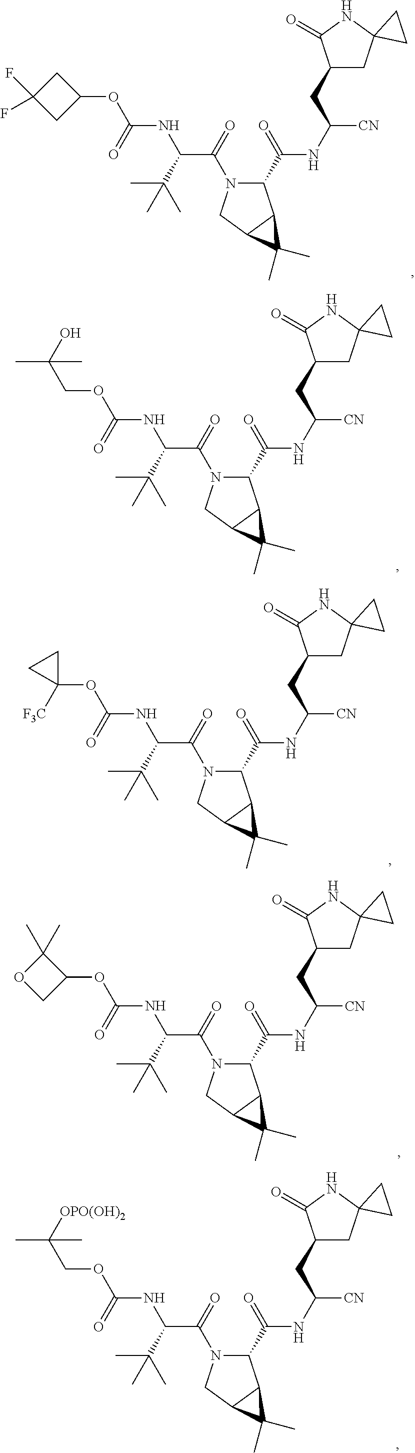 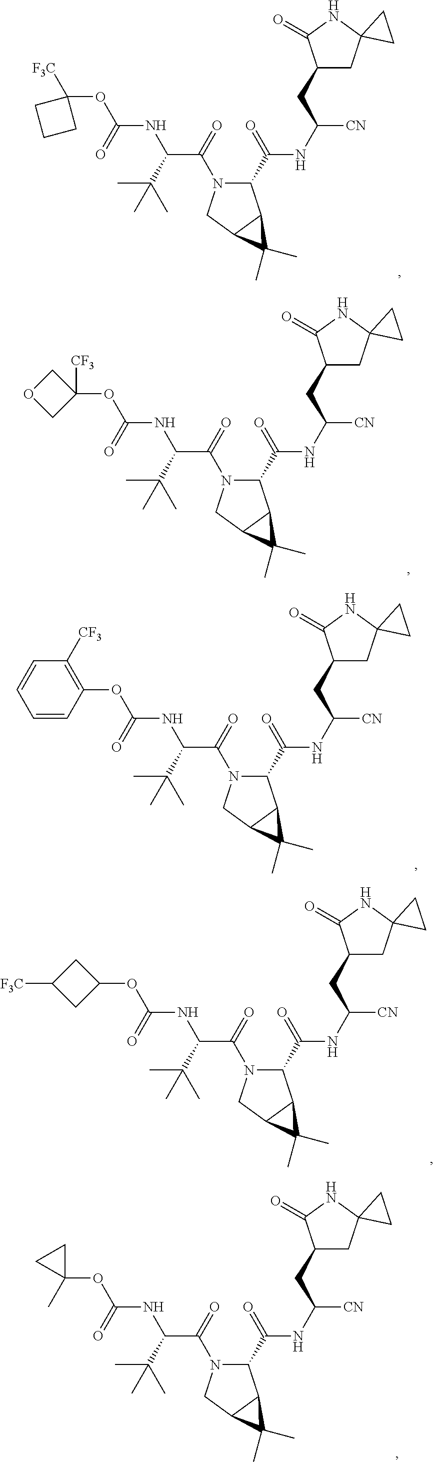 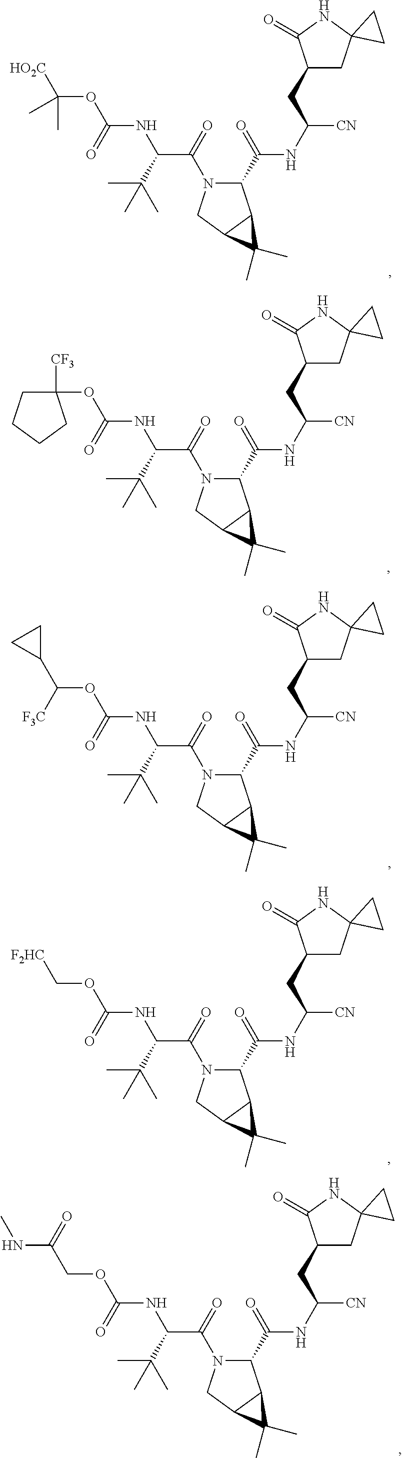 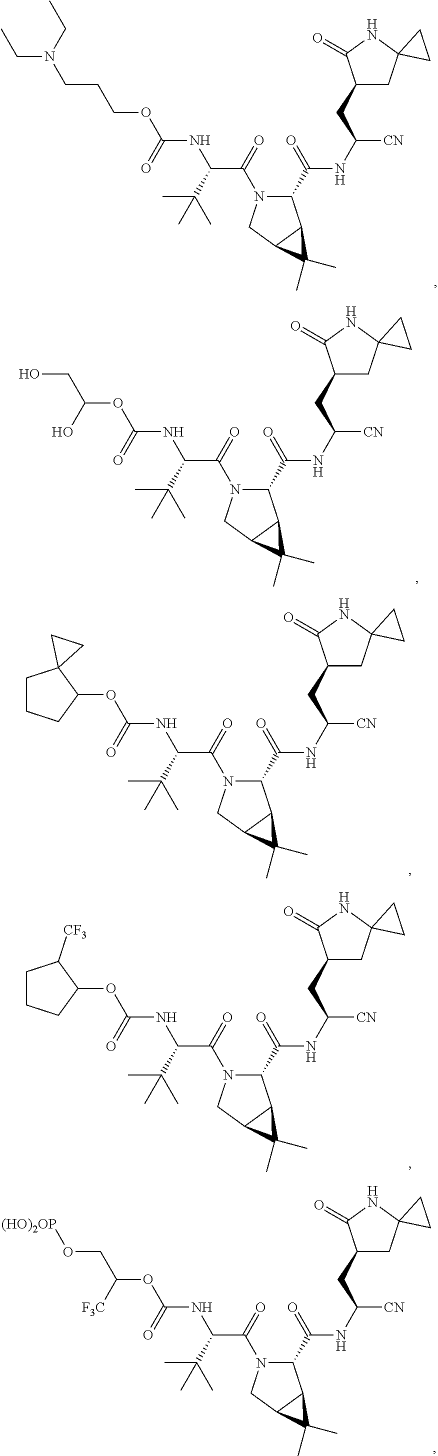 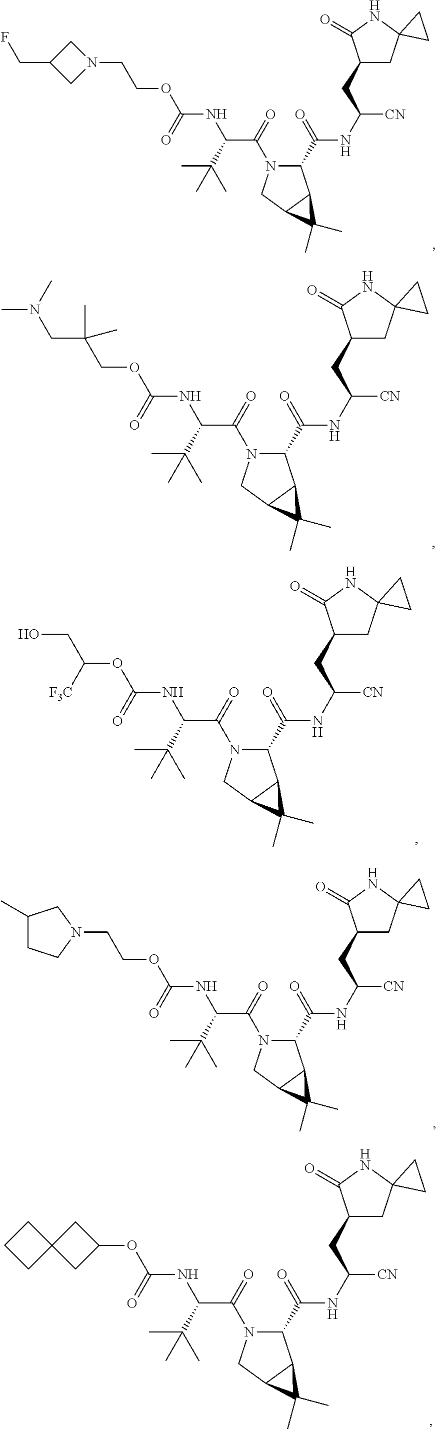 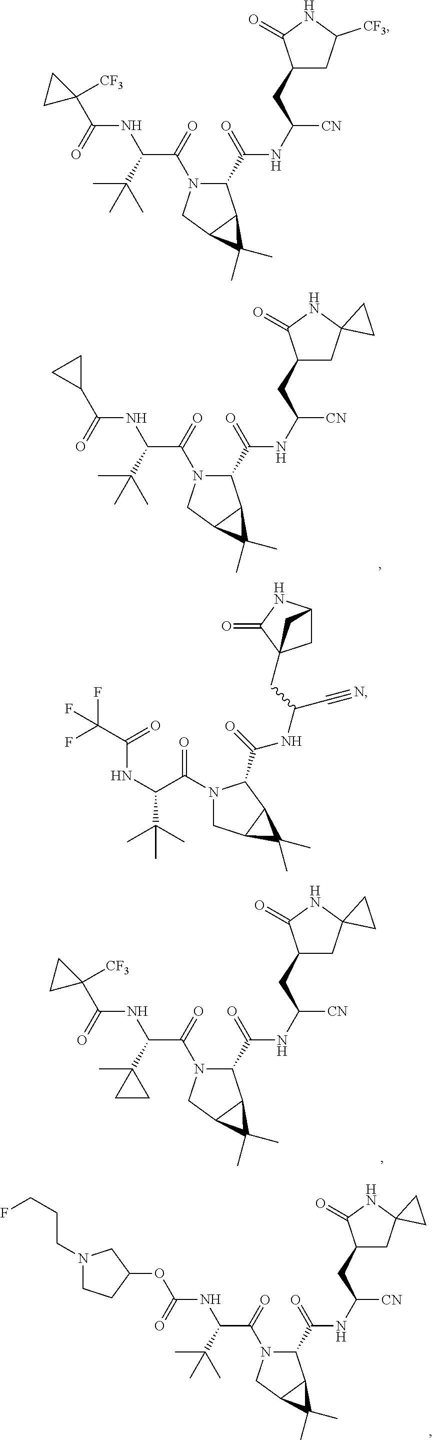 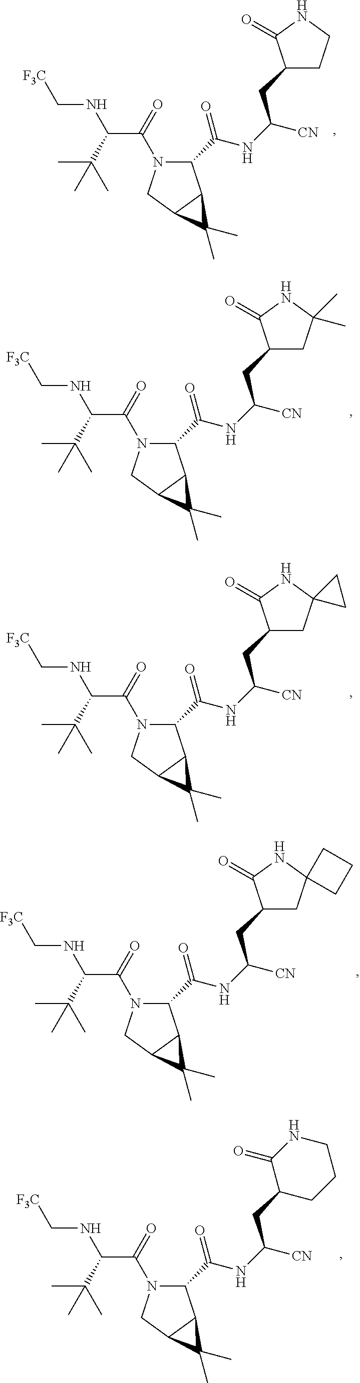 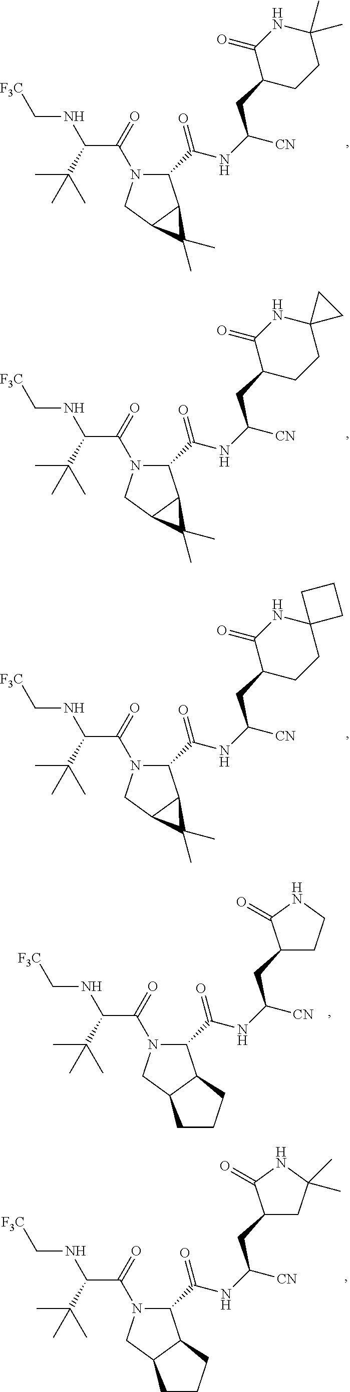 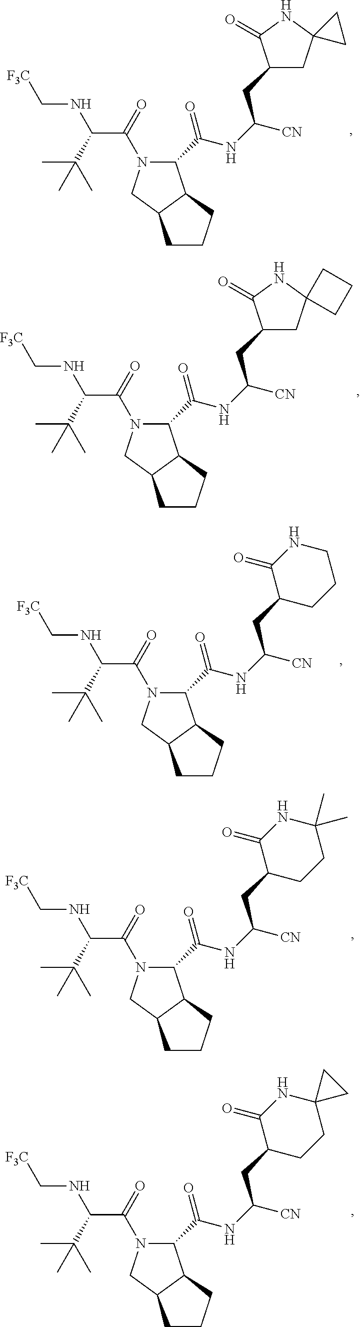 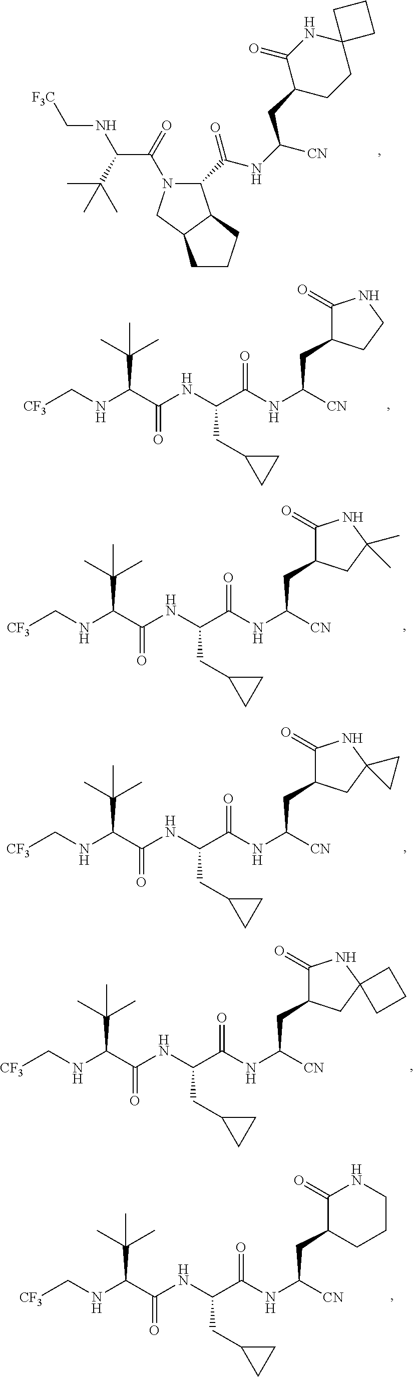 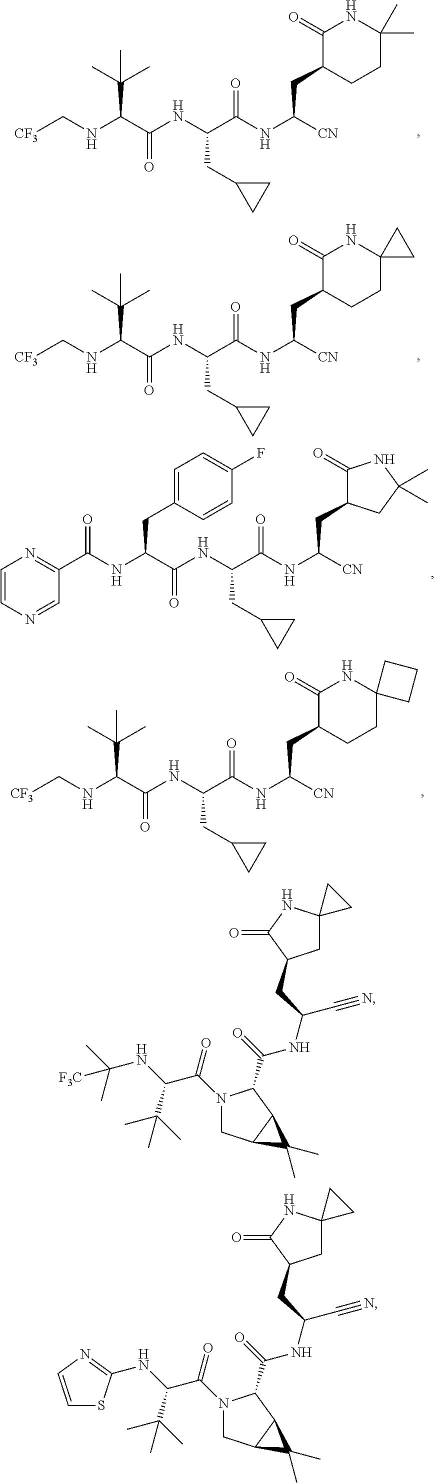 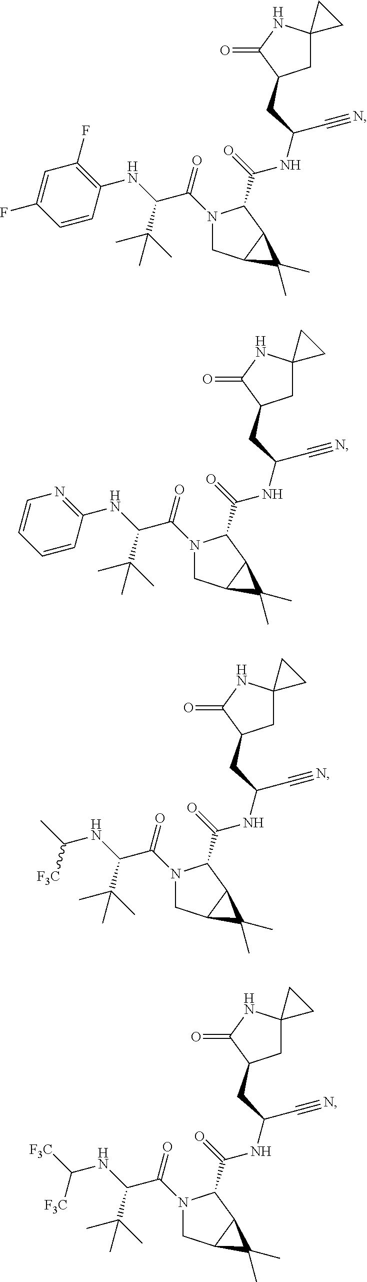 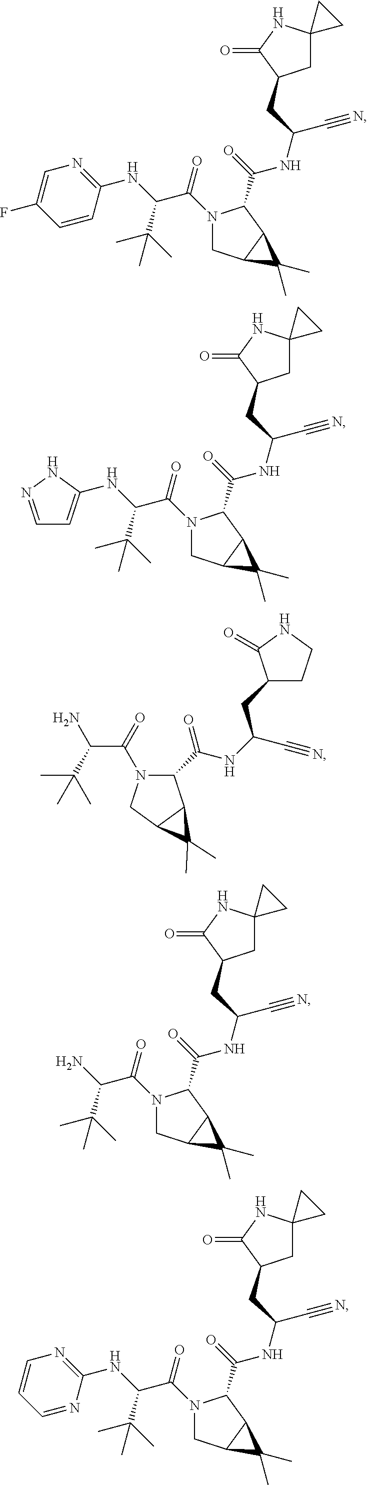 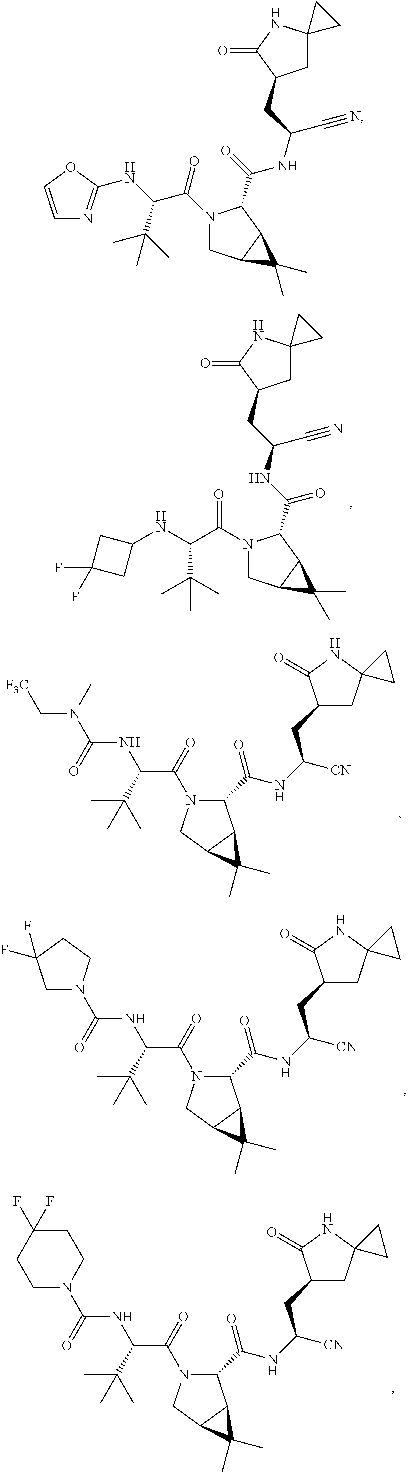 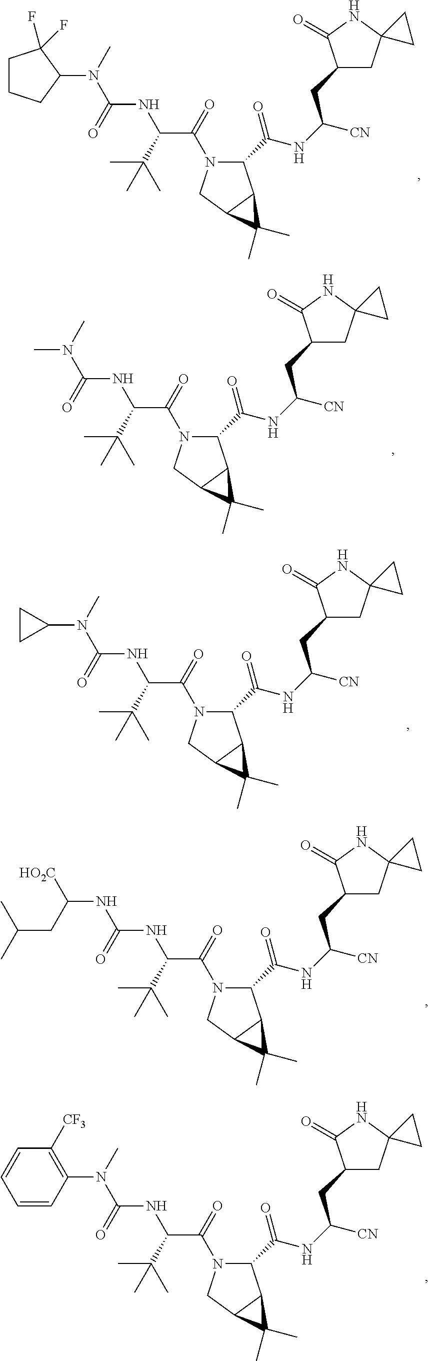 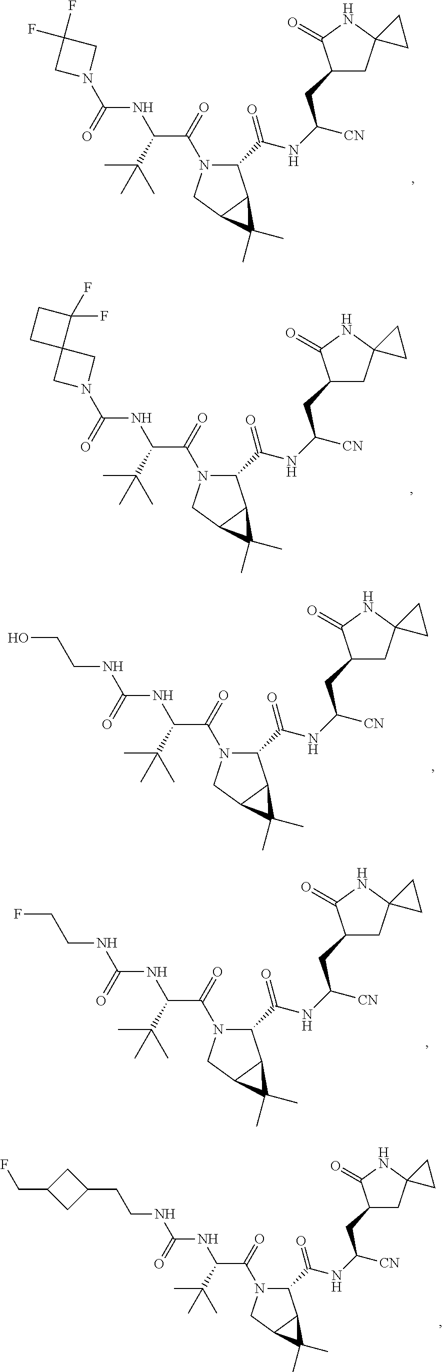 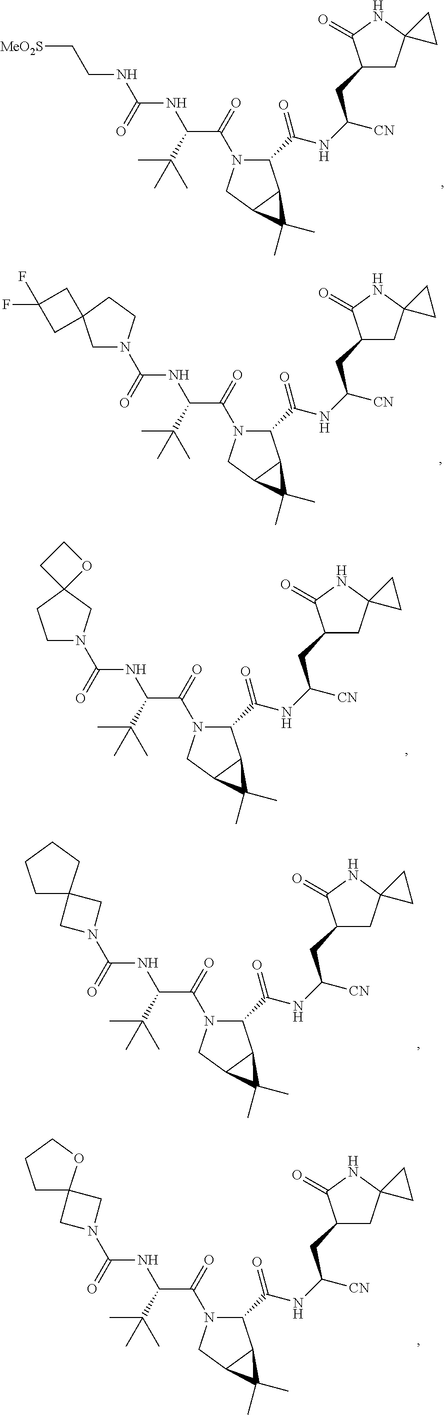 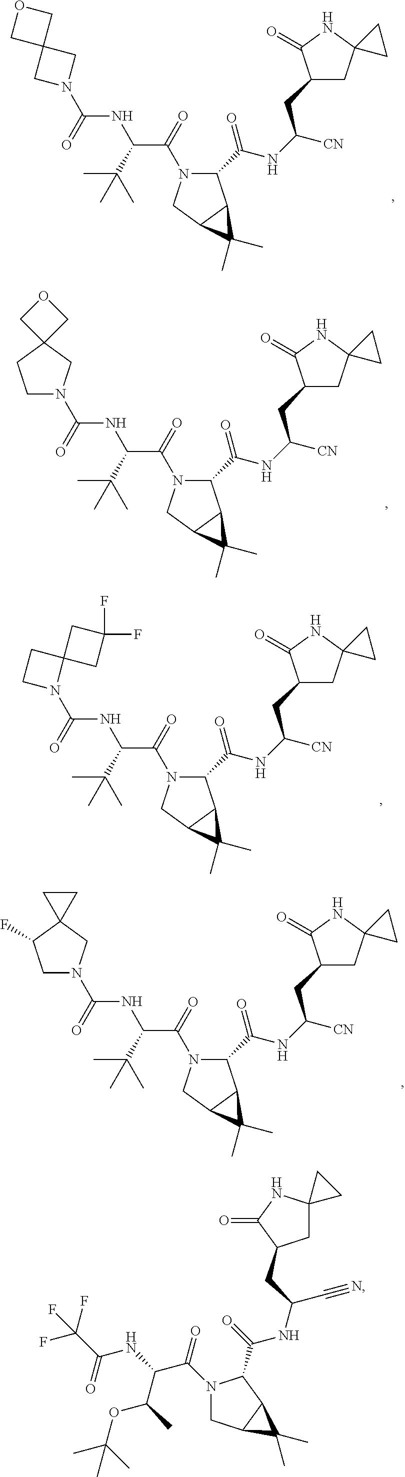 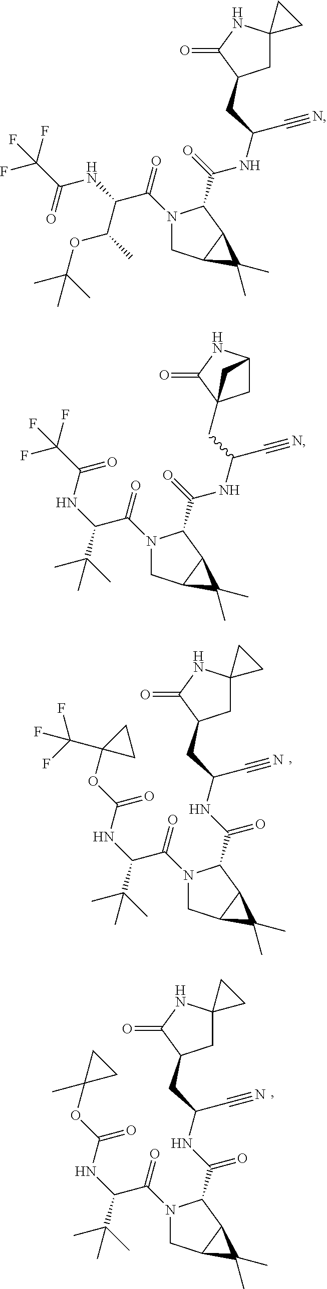 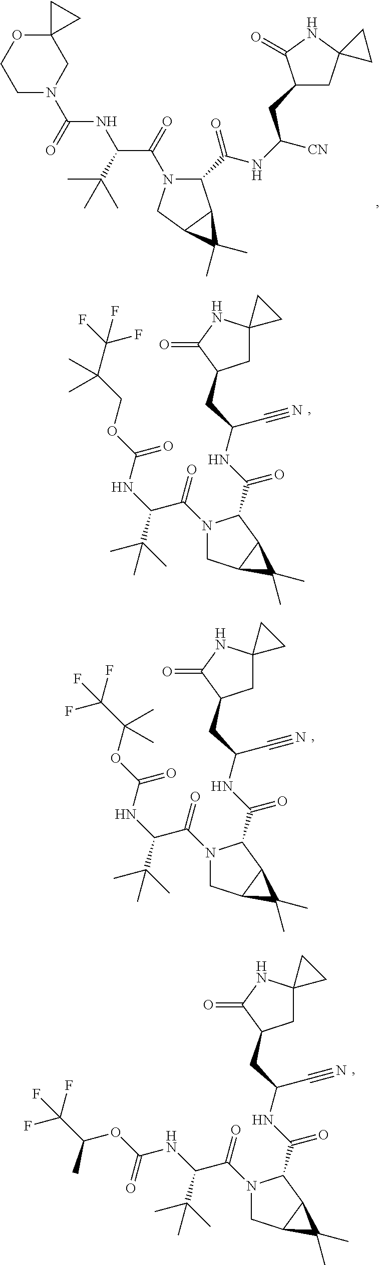 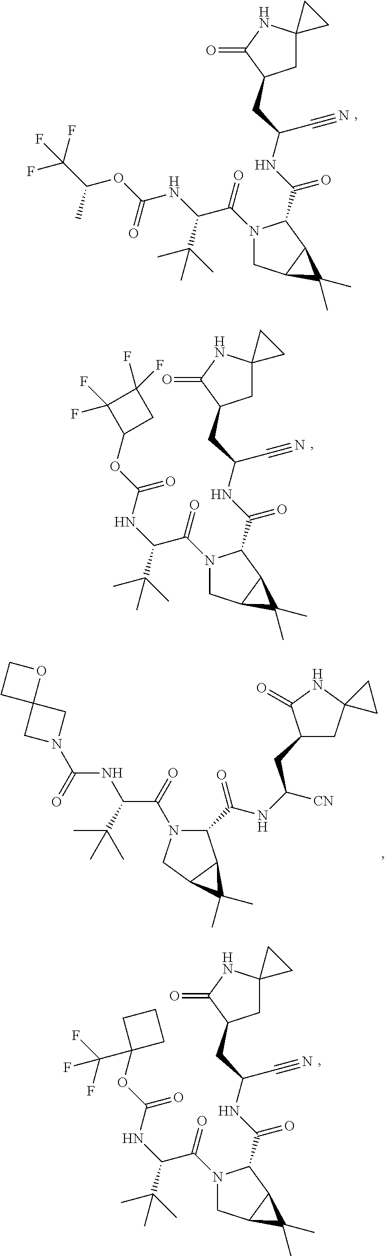 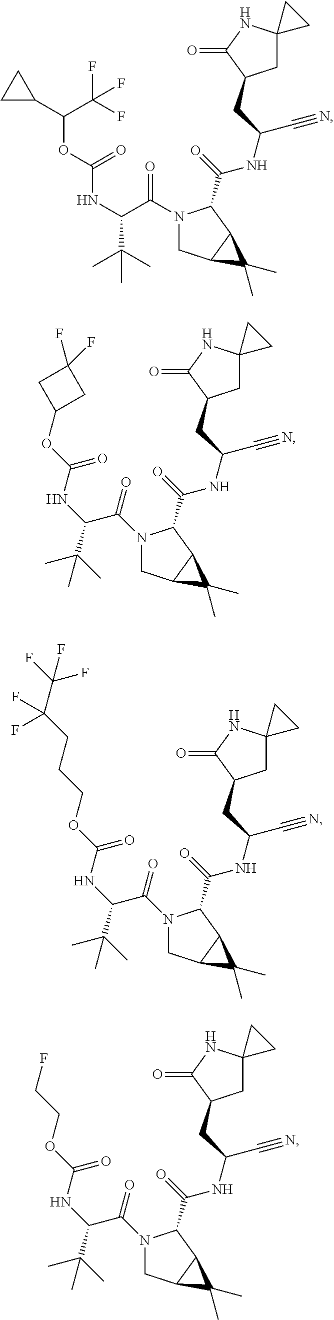 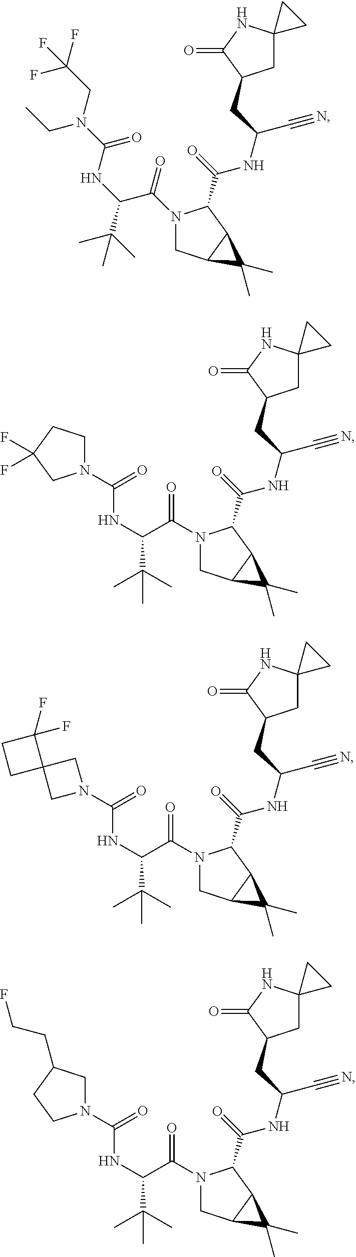 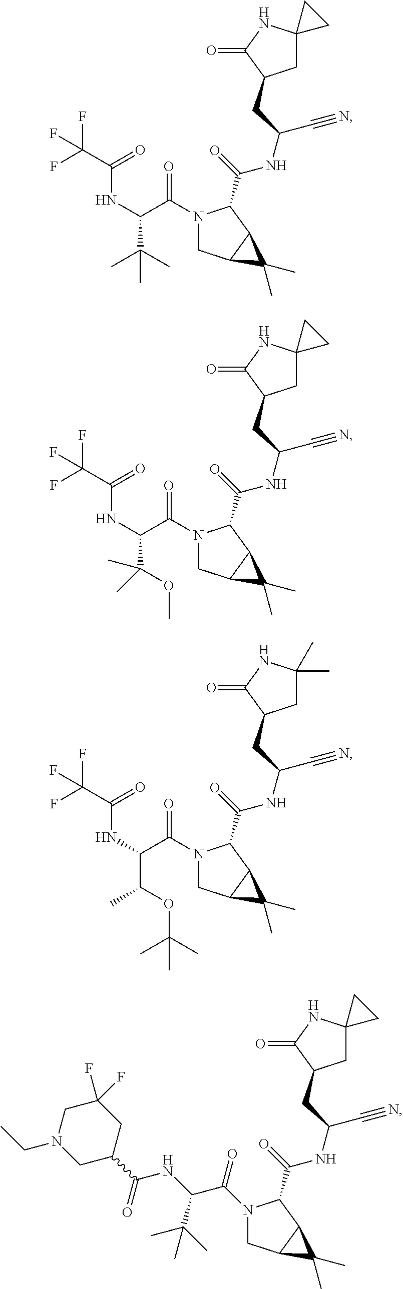 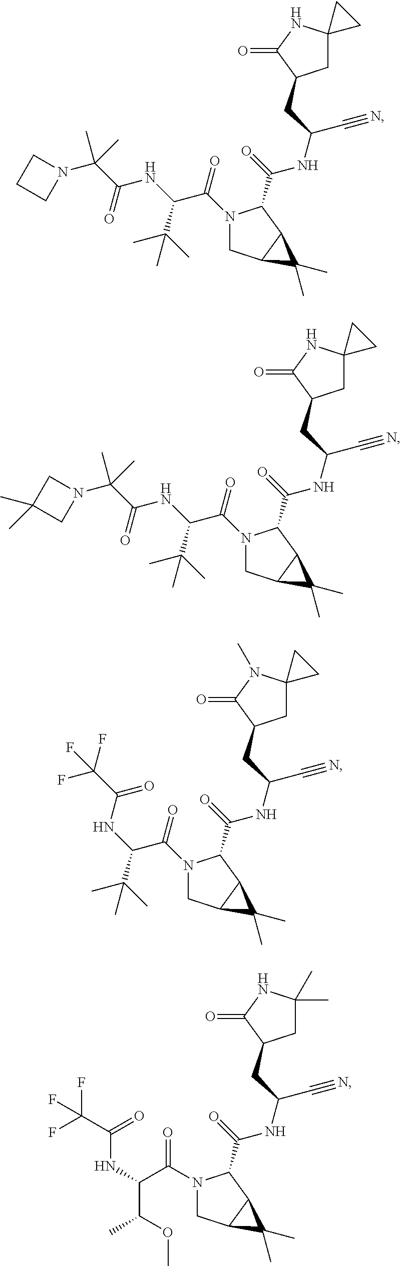 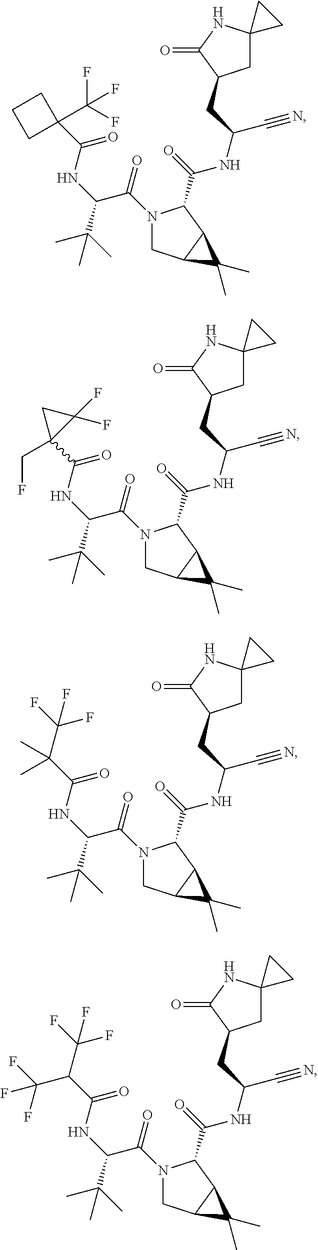 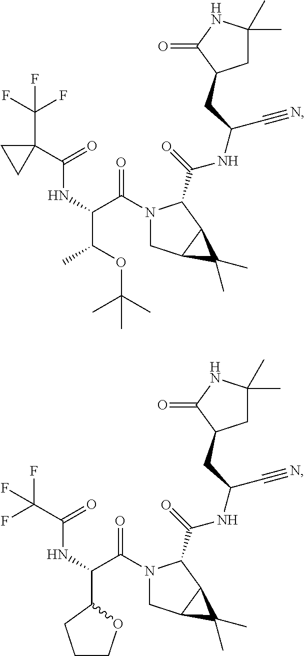 [SMILES: C[C@@H](OC(C)(C)C)[C@H](NC(=O)C1(C(F)(F)F)CC1)C(=O)N1C[C@H]2[C@@H]([C@H]1C(=O)N[C@H](C#N)C[C@@H]1CC(C)(C)NC1=O)C2(C)C ; CC1(C)C[C@@H](C[C@@H](C#N)NC(=O)[C@@H]2[C@@H]3[C@H](CN2C(=O)[C@@H](NC(=O)C(F)(F)F)C2CCCO2)C3(C)C)C(=O)N1] 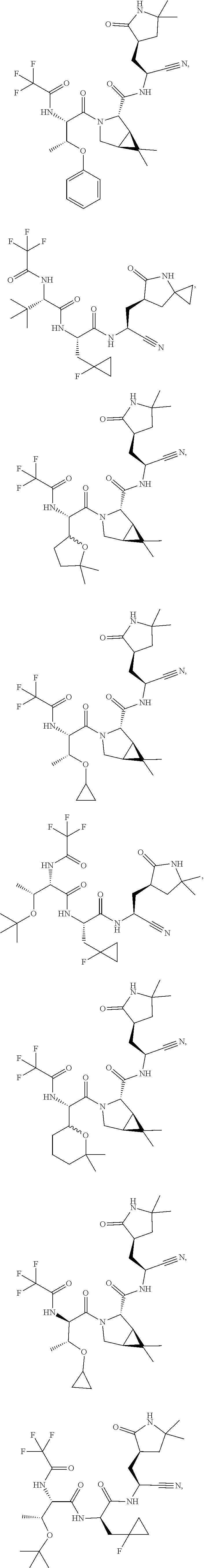 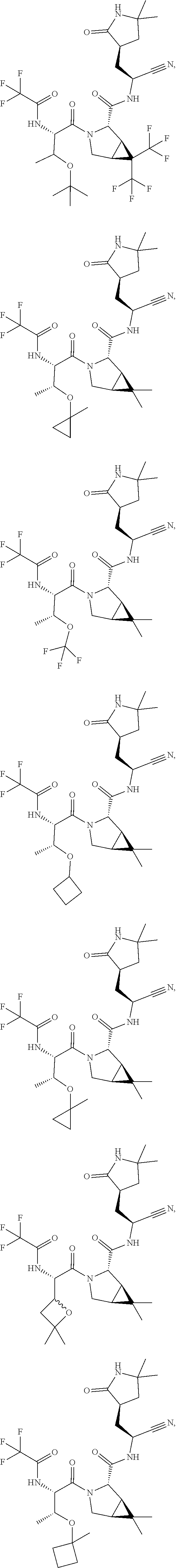 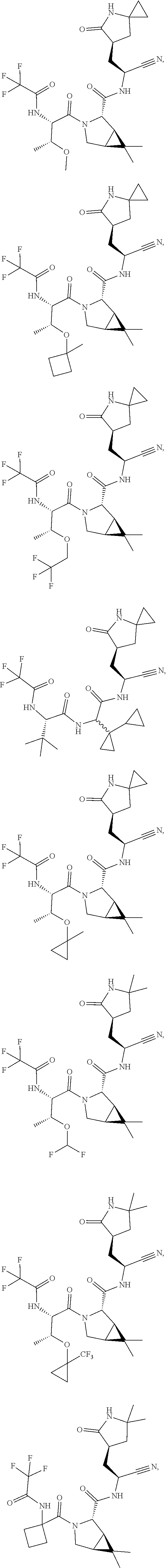 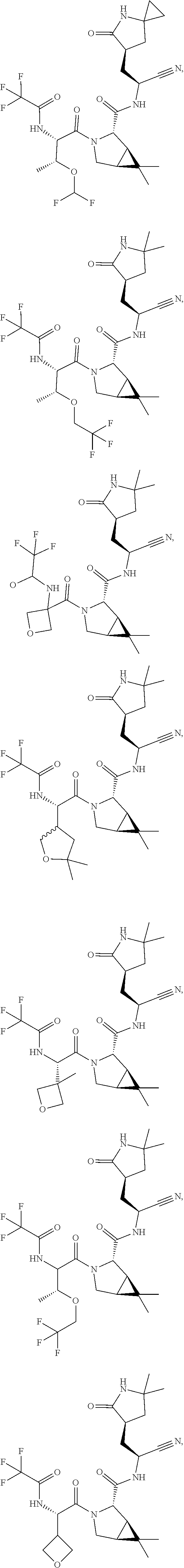 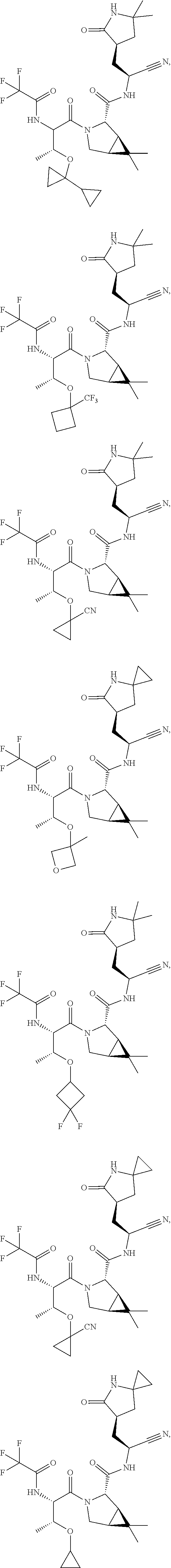 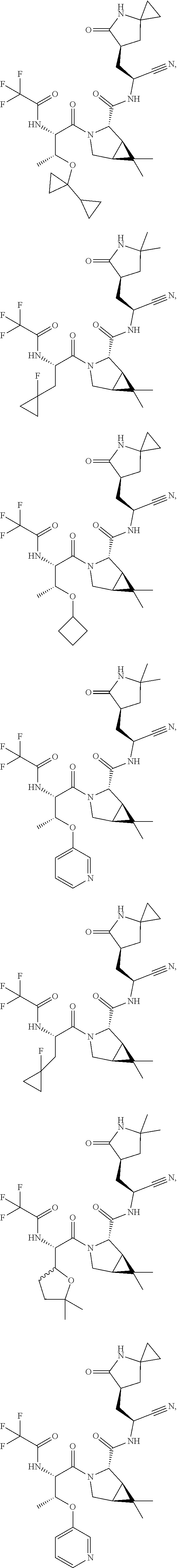 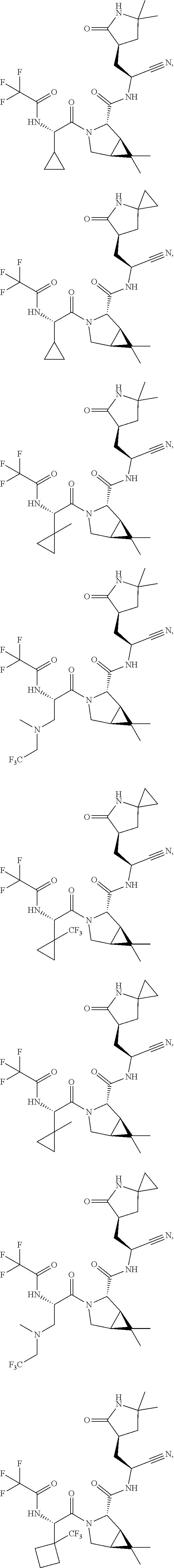 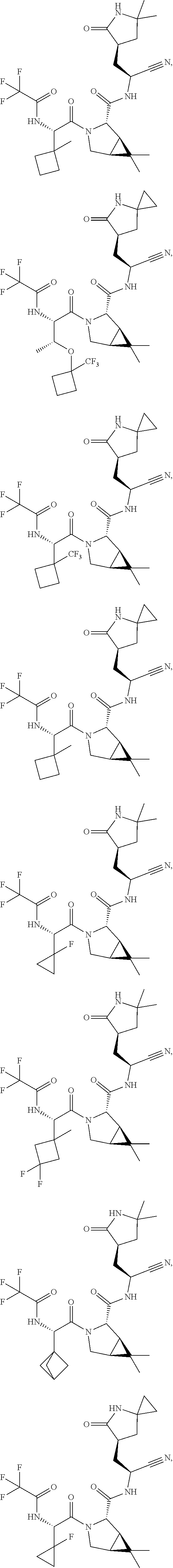 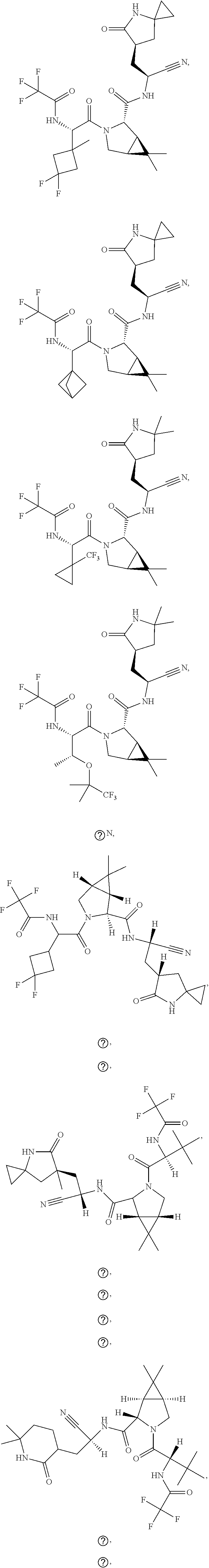 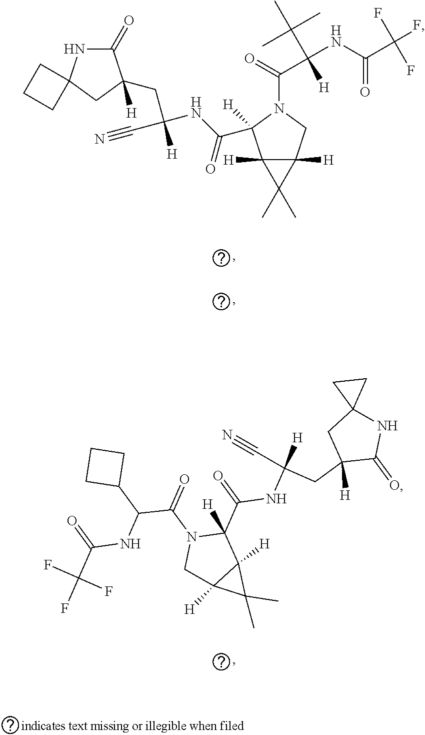 [SMILES: CC(C)(C)[C@H](NC(=O)C(F)(F)F)C(=O)N1C[C@H]2[C@@H]([C@H]1C(=O)N[C@H](C#N)C[C@@H]1CC3(CCC3)NC1=O)C2(C)C ; CC1(C)[C@@H]2[C@@H](C(=O)N[C@H](C#N)C[C@@H]3CC4(CC4)NC3=O)N(C(=O)C(NC(=O)C(F)(F)F)C3CCC3)C[C@@H]21] 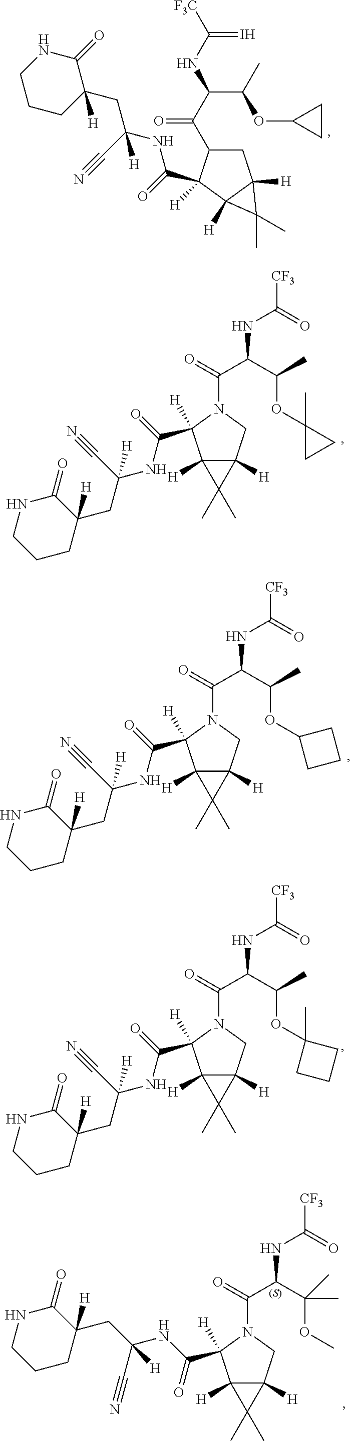 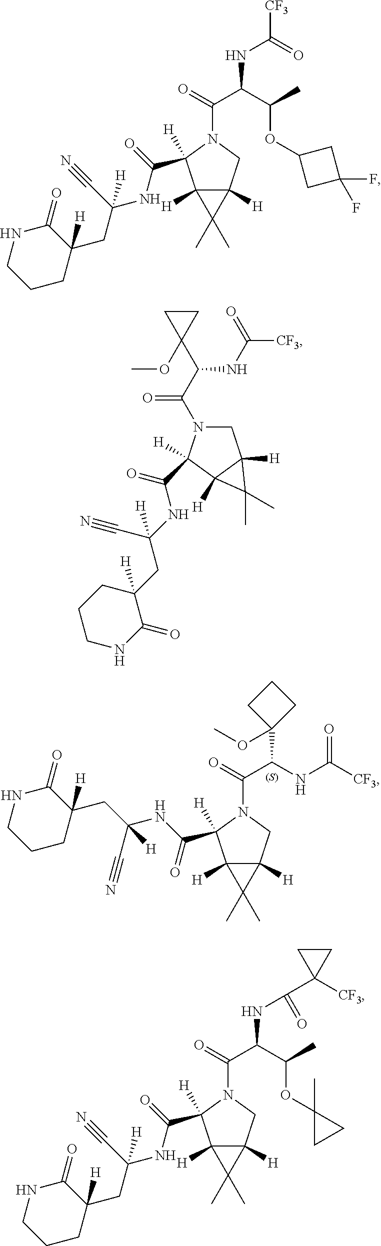 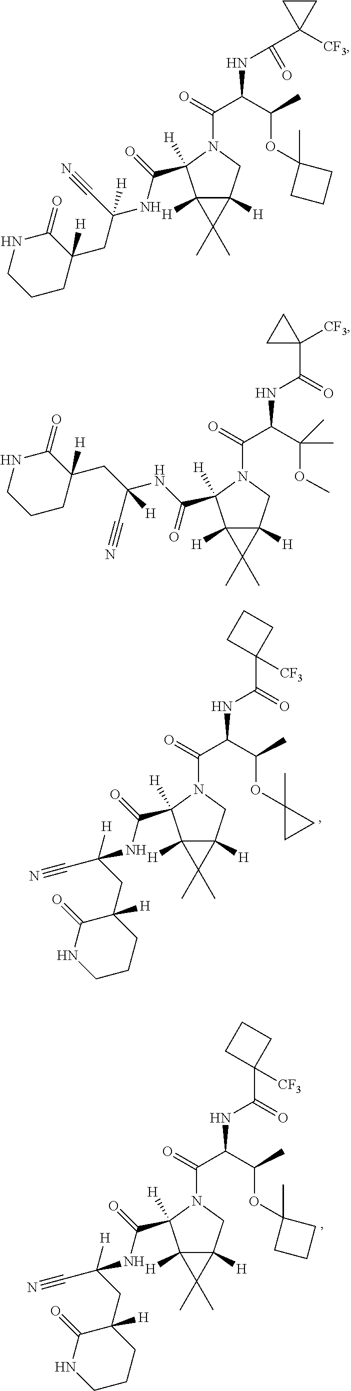 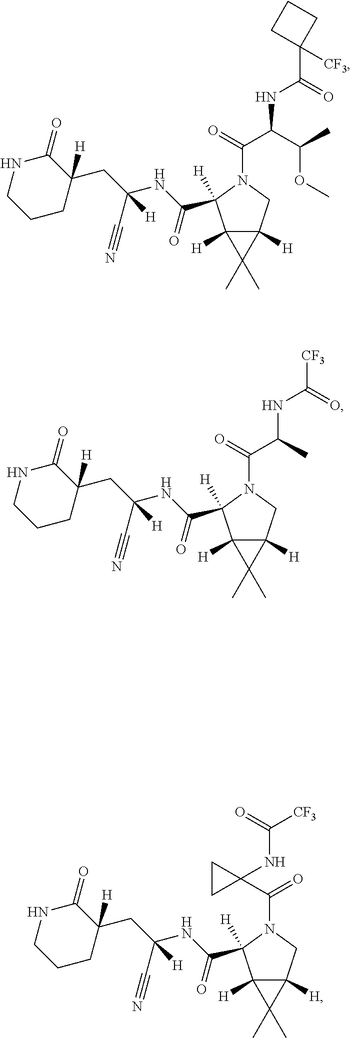 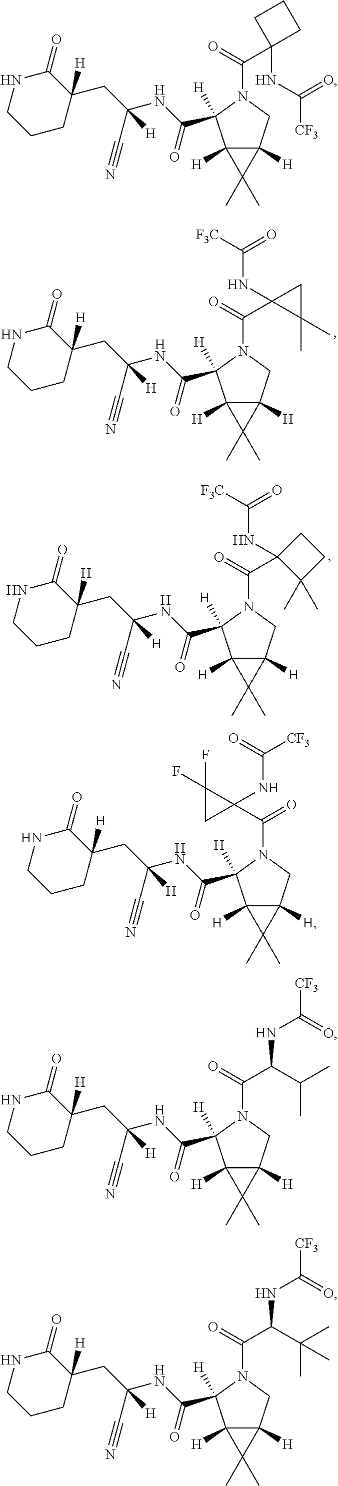 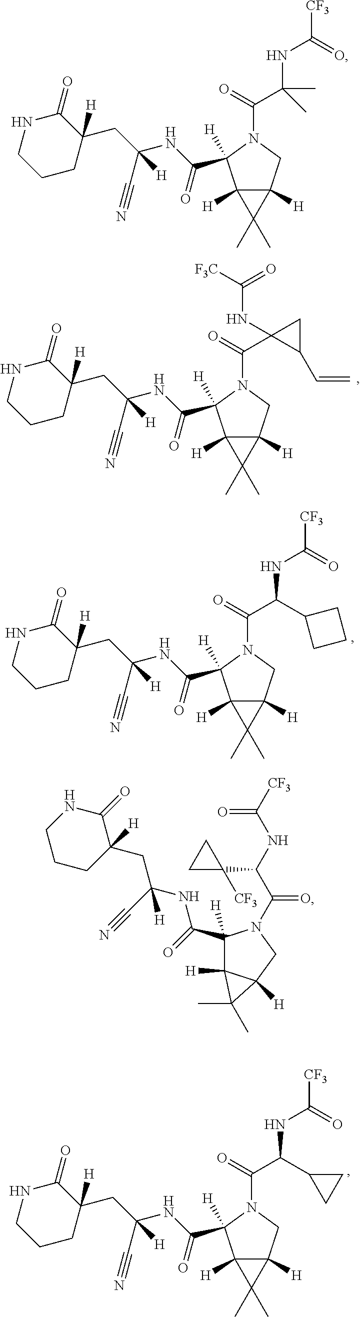 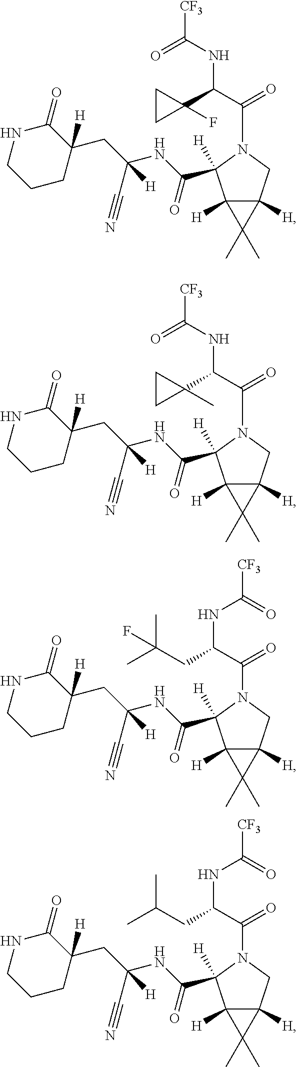 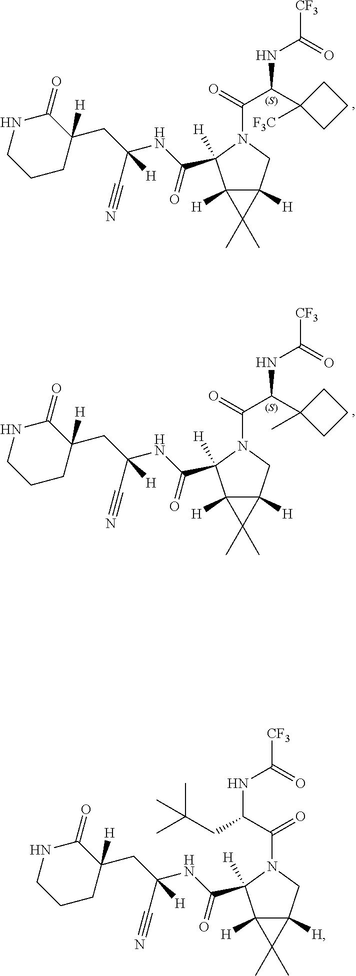 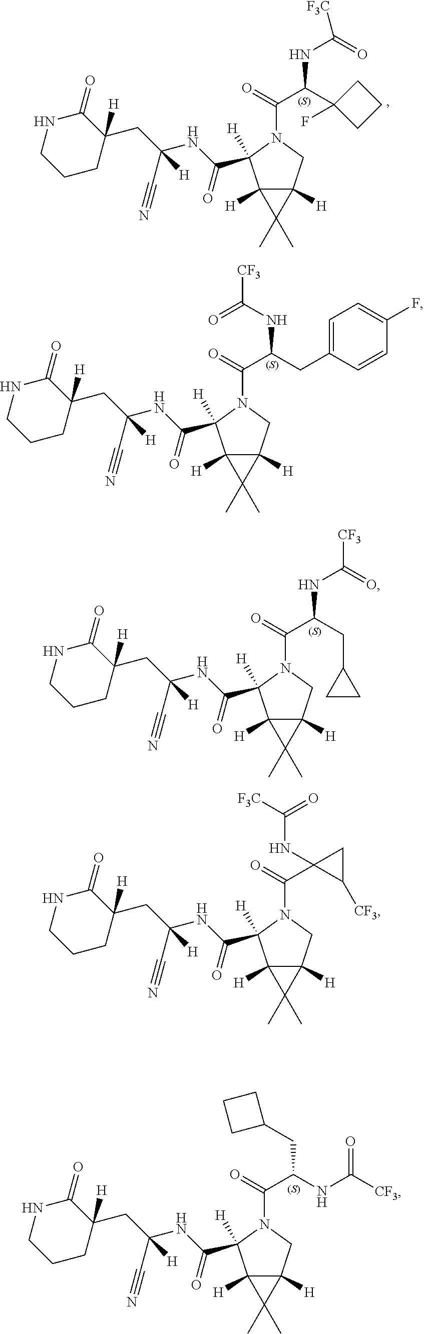 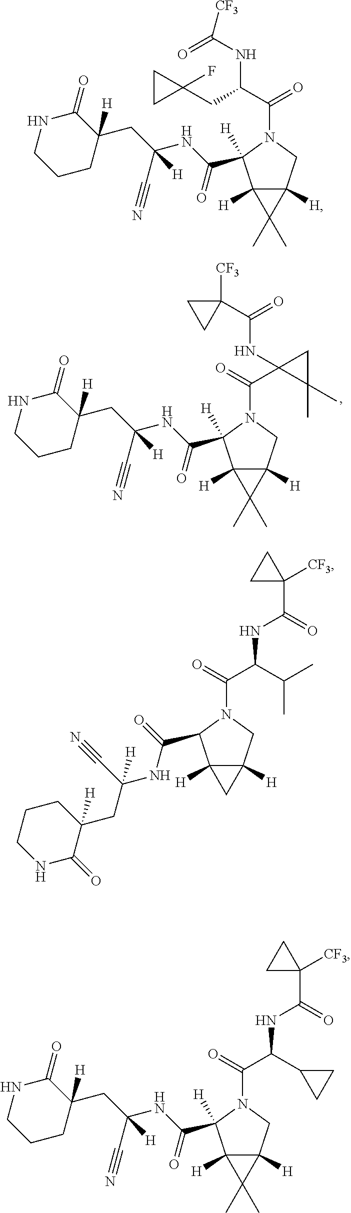 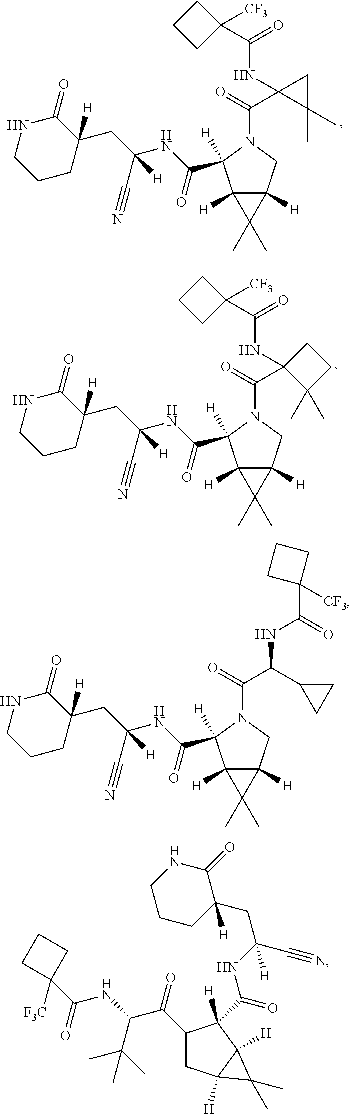 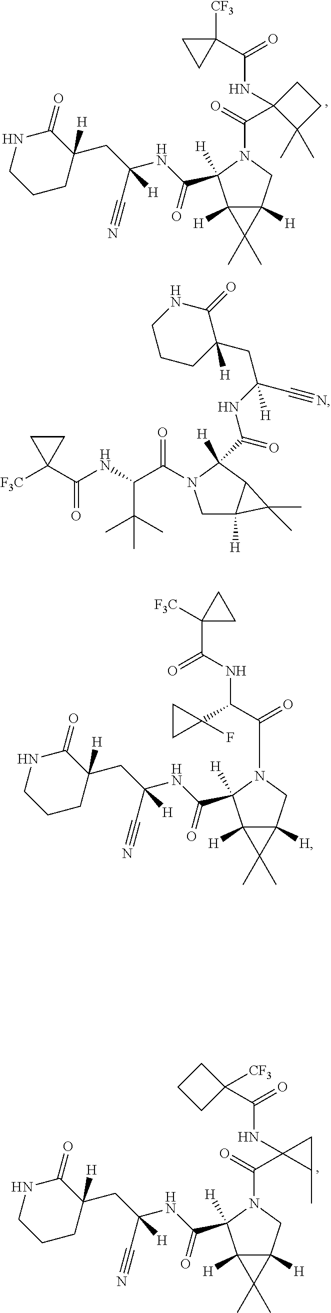 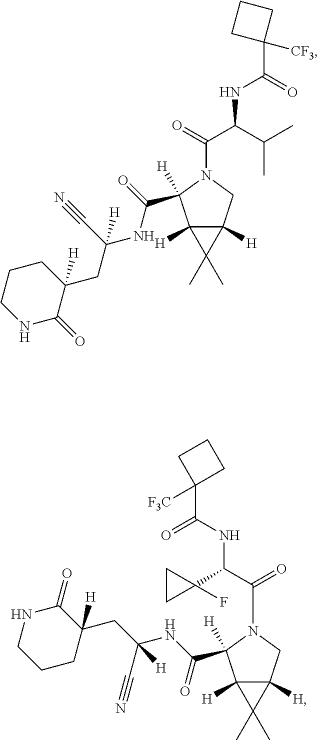
and pharmaceutically acceptable salts thereof.

55. A pharmaceutical composition comprising a compound of any one of claims 1-54 and a pharmaceutically acceptable excipient.

56. A method of ameliorating or treating a viral infection in a patient in need thereof, comprising administering to the patient a therapeutically effective amount of a compound of any one of claims 1-54.

57. The method of claim 56, wherein the viral infection is from a virus selected from the group consisting of an RNA virus, a DNA virus, a coronavirus, a papillomavirus, a pneumovirus, a picomavirus, an influenza virus, an adenovirus, a cytomegalovirus, a polyomavirus, a poxvirus, a flavivirus, an alphavirus, an ebola virus, a morbillivirus, an enterovirus, an orthopneumovirus, a lentivirus, an arenavirus, a herpes virus, and a hepatovirus.

58. The method of claim 56, wherein the viral infection is from a virus selected from the group consisting of Norwalk virus, feline calicivirus, MD 145, murine norovirus, an astrovirus, vesicular exanthema of swine virus, rabbit hemorrhagic disease virus, enterovirus (EV)-68 virus, EV-71 virus, poliovirus, coxsackievirus, foot-and-mouth disease virus, hepatitis A, porcine teschovirus, rhinovirus, human coronavirus, transmissible gastroenteritis virus, murine hepatitis virus, bovine coronavirus, feline infectious peritonitis virus, and severe acute respiratory syndrome coronavirus.

59. The method of any one of claims 56-58, wherein the viral infection is a coronavirus infection.

60. The method of any one of claims 56-59, wherein the viral infection is a coronavirus selected from the group consisting of: 229E alpha coronavirus, NL63 alpha coronavirus, OC43 beta coronavirus, HKU1 beta coronavirus, Middle East Respiratory Syndrome (MERS) coronavirus (MERS-CoV), severe acute respiratory syndrome (SARS) coronavirus (SARS-CoV), and SARS-CoV-2 (COVID-19).

61. The method of any one of claims 56-58, wherein the viral infection is SARS-CoV-2.

62. The method of claim 55 or 56, wherein the viral infection is an arenavirus infection.

63. The method of claim 62, wherein the arenavirus is selected from the group consisting of: Junin virus, Lassa virus, Lujo virus, Machupo virus, and Sabia virus.

64. The method of claim 55 or 56, wherein the viral infection is an influenza infection.

65. The method of claim 64, wherein the influenza is influenza H1N1, H3N2 or H5N1.

66. A method of inhibiting transmission of a virus, a method of inhibiting viral replication, a method of minimizing expression of viral proteins, or a method of inhibiting virus release, comprising administering a therapeutically effective amount of a compound of any one of claims 1-54 to a patient suffering from the virus, and/or contacting an effective amount of a compound of any one of claims 1-54 with a virally infected cell.

67. The method of any one of claims 55-66, further comprising administering another therapeutic.

68. The method of any one of claims 55-66, further comprising administering an additional anti-viral therapeutic.

69. The method of claim 68, wherein the additional anti-viral therapeutic is selected from the group consisting of ribavirin, favipiravir, ST-193, oseltamivir, zanamivir, peramivir, danoprevir, ritonavir, remdesivir, cobicistat, elvitegravir, emtricitabine, tenofovir, tenofovir disoproxil, tenofovir alafenamide hemifumarate, abacavir, dolutegravir, efavirenz, elbasvir, ledipasvir, glecaprevir, sofosbuvir, bictegravir, dasabuvir, lamivudine, atazanavir, ombitasvir, lamivudine, stavudine, nevirapine, rilpivirine, paritaprevir, simeprevir, daclatasvir, grazoprevir, pibrentasvir, adefovir, amprenavir, ampligen, aplaviroc, anti-caprine antibody, balavir, cabotegravir, cytarabine, ecoliever, epigallocatechin gallate, etravirine, fostemsavir, gemcitabine, griffithsin, imunovir, indinavir, maraviroc, methisazone, MK-2048, nelfmavir, nevirapine, nitazoxanide, norvir, plerixafor, PRO 140, raltegravir, pyramidine, saquinavir, telbivudine, TNX-355, valacyclovir, VIR-576, and zalcitabine.

70. The method of claim 67, wherein the another therapeutic is selected from the group consisting of protease inhibitors, fusion inhibitors, M2 proton channel blockers, polymerase inhibitors, 6-endonuclease inhibitors, neuraminidase inhibitors, reverse transcriptase inhibitor, aciclovir, acyclovir, protease inhibitors, arbidol, atazanavir, atripla, boceprevir, cidofovir, combivir, darunavir, docosanol, edoxudine, entry inhibitors, entecavir, famciclovir, fomivirsen, fosamprenavir, foscarnet, fosfonet, ganciclovir, ibacitabine, immunovir, idoxuridine, imiquimod, inosine, integrase inhibitor, interferons, lopinavir, loviride, moroxydine, nexavir, nucleoside analogues, penciclovir, pleconaril, podophyllotoxin, ribavirin, tipranavir, trifluridine, trizivir, tromantadine, truvada, valaciclovir, valganciclovir, vicriviroc, vidarabine, viramidine, and zodovudine.

71. The method of claim 69, wherein the additional anti-viral therapeutic is selected from the group consisting of lamivudine, an interferon alpha, a VAP anti-idiotypic antibody, enfuvirtide, amantadine, rimantadine, pleconaril, aciclovir, zidovudine, fomivirsen, a morpholino, a protease inhibitor, double-stranded RNA activated caspase oligomerizer (DRACO), rifampicin, zanamivir, oseltamivir, danoprevir, ritonavir, remdesivir, cobicistat, elvitegravir, emtricitabine, tenofovir, tenofovir disoproxil, tenofovir alafenamide hemifumarate, abacavir, dolutegravir, efavirenz, elbasvir, ledipasvir, glecaprevir, sofosbuvir, bictegravir, dasabuvir, lamivudine, atazanavir, ombitasvir, lamivudine, stavudine, nevirapine, rilpivirine, paritaprevir, simeprevir, daclatasvir, grazoprevir, pibrentasvir, adefovir, amprenavir, ampligen, aplaviroc, anti-caprine antibody, balavir, cabotegravir, cytarabine, ecoliever, epigallocatechin gallate, etravirine, fostemsavir, gemcitabine, griffithsin, imunovir, indinavir, maraviroc, methisazone, MK-2048, nelfmavir, nevirapine, nitazoxanide, norvir, plerixafor, PRO 140, raltegravir, pyramidine, saquinavir, telbivudine, TNX-355, valacyclovir, VIR-576, and zalcitabine.

72. A method of prophylactically treating a patient at risk of viral infection, comprising administering to the patient an effective amount of a compound of any one of claims 1-54.

73. The method of claim 72, wherein the compound is administered before viral exposure.

74. The method of claim 72 or 73, wherein the compound is administered after viral exposure.
